(19) United States
(12) Patent Application Publication
(10) Pub. No.: US 2024/0109917 A1
(43) Pub. Date: Apr. 4, 2024

(54) PYRAZOLOTHIAZOLE CARBOXAMIDES AND THEIR USES AS PDGFR INHIBITORS

(71) Applicant: ACTELION PHARMACEUTICALS LTD, Allschwil (CH)

(72) Inventors: David BAUMAN, Raritan, NJ (US); Zhijie LIU, Raritan, NJ (US); Tianbao LU, Raritan, NJ (US); Bin ZHU, Raritan, NJ (US); Van NGUYEN, Raritan, NJ (US); Marchello CAVITT, Raritan, NJ (US); Michael J. HAWKINS, Raritan, NJ (US)

(21) Appl. No.: 18/258,675

(22) PCT Filed: Dec. 22, 2021
(86) PCT No.: PCT/EP2021/087215

§ 371 Date: Jun. 21, 2023

Related U.S. Application Data
(60) Provisional application No. 63/130,135, filed on Dec. 23, 2020.
(60) Provisional application No. 63/245,587, filed on Sep. 17, 2021.

(51) Int. Cl. C07D 519/00; C07D 513/04

(52) U.S. Cl. CPC C07D 519/00; C07D 513/04

(57) ABSTRACT

The disclosure is directed to compounds of formula (I), and pharmaceutically acceptable salts thereof. Pharmaceutical compositions comprising compounds of formula (I), as well as methods of their use and preparation, are also described.

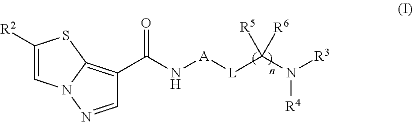

### TECHNICAL FIELD

The disclosure is directed to PDGFR inhibitors and methods of their use.

### BACKGROUND

Protein kinases are a family of enzymes that catalyze the phosphorylation of specific residues in proteins. Protein kinases play a critical role in the control cell growth, proliferation, differentiation, metabolism, apoptosis, cell mobility, transcription, translation and other signaling processes. The overexpression or inappropriate expression of protein kinases plays a significant role in the development of many diseases and disorders including central nervous system disorders, inflammatory disorders, metabolic disorders, autoimmune diseases, cardiovascular diseases, fibrotic diseases, transplantation rejection, cancer and infectious diseases.

Growth factors (GF) are important regulators of human homeostasis involved in maintaining a delicate balance between cell growth, differentiation, and proliferation. Consequently, dysregulation of GF signaling are implicated in many diseases including oncology, immunology, fibroproliferative, cardiovascular, vascular disorders and pulmonary hypertension. GF bind to several different receptors that amplify the signal through activation of the specific receptor through phosphorylation, leading to confirmation changes increasing the affinity for ATP and the phosphorylation of downstream proteins leading to activation of several signaling cascades. Therefore, small changes in GF or the cognate receptors can significantly alter the local signaling and have dramatic effects on initiation and progression of many diseases.

Platelet-derived growth factor (PDGF) is one of many GFs that regulate cell growth and division. PDGF exerts its biological responses via activation of two highly specific, transmembrane receptor tyrosine kinases, termed PDGFR α and PDGFR β, which can form three different dimeric receptors—αα, ββ and αβ. These receptors can interact with the different dimeric PDGF ligands (PDGF-AA, PDGF-BB, PDGF-CC, PDGF-DD and PDGF-AB) with different specificities and efficacies. The receptors are activated by ligand-induced dimerization, leading to autophosphorylation on specific tyrosine residues. PDGFR phosphorylation recruits signaling proteins containing Tyr(P)-binding domains. Several of these signaling proteins include Src kinase family members, phospholipase C-y1, the p38a subunit of PI3K, GTPase-activating protein. The formation of receptor-signaling complexes then initiates the activation of various signaling pathways, including the Ras-mitogen activated protein (MAP) kinase pathway, the PI3kinase-Akt pathway, the PLC-y1 and the Src pathway. Activation of PDGFRα or PDGFRβ by PDGFs, leads to protein synthesis, proliferation, migration, protection against apoptosis and cellular transformation, key mechanisms associated with several vascular diseases including pulmonary hypertension. Platelet-derived growth factor (PDGF) and its receptors (PDGFR), including PDGFRα and PDGFRβ, play important roles in tumorigenesis, tumor progression, and the regulation of stromal cell function. Constitutive activation of PDGFR signaling, gene rearrangement, and activating mutations of PDGFR have been identified in various types of human tumors and malignancies.

PDGFR signaling is implicated in the development and progression of pulmonary hypertension. PDGFs are expressed in ECs, SMCs and macrophages and are strong mitogens and chemokines. Increased signaling through PDGFRβ leads to smooth muscle cell proliferation which contributes to the development of vascular remodeling. PDGF and PDGF receptors (α and β) are upregulated in human and animals with pulmonary hypertension. Preclinically, efficacy in preventing and reversing vascular remodeling in experimentally induced pulmonary hypertension was demonstrated through non-selective inhibition of PDGF receptors. Clinically, imatinib (also known as Gleevec), a non-selective tyrosine kinase inhibitor including PDGF receptors improved exercise capacity and hemodynamics in patients with advanced pulmonary hypertension. Conversely, dasatinib, a receptor tyrosine kinases inhibitor, was linked to cardiotoxicity and the development of pulmonary hypertension, emphasizing the importance of the appropriate kinase selectivity, and associated differentiated profile.

A need exists for additional PDGFR inhibitors for the treatment of pulmonary hypertension and other conditions in which PDGFR signaling is implicated.

### SUMMARY

The present disclosure provides PDGFR inhibitors.

In some aspects, the present disclosure provides compounds of formula (I₀):

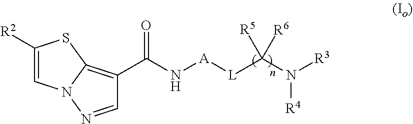

or pharmaceutically acceptable salts thereof, wherein
A is an optionally substituted phenyl ring, an optionally substituted pyridinyl ring, or an optionally substituted 5-membered heteroaryl ring containing 1-2 heteroatoms that are each independently O, N, or S;
R² is optionally substituted aryl, optionally substituted heteroaryl, optionally substituted fused heterocycloalkyl, optionally substituted alkyl, optionally substituted alkenyl, optionally substituted cycloalkyl, or optionally substituted heterocycloalkyl;
R³ and R⁴ are each independently optionally substituted aryl, optionally substituted heteroaryl, optionally substituted alkyl, optionally substituted cycloalkyl, or optionally substituted heterocycloalkyl; or one of R³ or R⁴ may be H; or
   R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N;
each R⁵ and each R⁶ is independently H, C₁-C₆alkyl, or C₃-C₅cycloalkyl; or
   an R⁵ and R⁶ attached to the same carbon atom, together with that carbon atom, may form a C₃-C₆cycloalkyl ring; or
   an R⁵ and R⁶ attached to the same carbon atom, together with that carbon atom, may form a C═O; or
   an R⁵ or R⁶, together with an R³ or R⁴ may form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system;
n is 1, 2, 3, 4, or 5; and
L is —NHC(O)— or

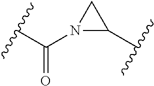 [SMILES: *C(=O)N1CC1*]

when n is 1; or —NHC(O)—, —NHS(O)₂—,

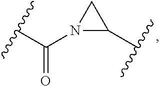 [SMILES: *C(=O)N1CC1*]

—NHC(O)O—, —S(O)₂NH—, —C(O)NH—, or —NHC(O)NH when n is 2,3,4, or 5.

In some embodiments, the present disclosure provides compounds of formula (I):

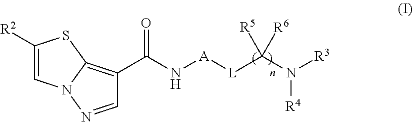

or pharmaceutically acceptable salts thereof, wherein A is an optionally substituted phenyl ring, an optionally substituted pyridinyl ring, or an optionally substituted 5-membered heteroaryl ring containing 1-2 heteroatoms that are each independently O, N, or S; R² is optionally substituted aryl, optionally substituted heteroaryl, optionally substituted alkyl, optionally substituted cycloalkyl, or optionally substituted heterocycloalkyl; R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; n is 1, 2, or 3; and when n is 1, L is —NHC(O)—, and when n is 2 or 3, L is —C(O)NH—, —NHC(O)—, or —NHC(O)NH.

In some aspects, the present disclosure provides compounds of formula (IA) or formula (IB)

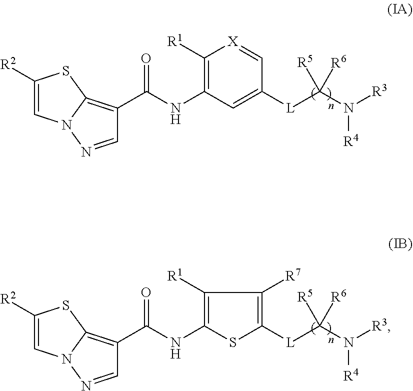

or pharmaceutically acceptable salts thereof, wherein R¹ is H, C₁-C₆alkyl, C₃-C₆cycloalkyl, halogen, —CN, or C₁-C₄fluoroalkyl; R² is optionally substituted aryl, optionally substituted heteroaryl, optionally substituted alkyl, optionally substituted cycloalkyl, or optionally substituted heterocycloalkyl; R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; R⁷ is H, C₁-C₆alkyl, C₃-C₆cycloalkyl, halogen, —CN, or —CF₃; n is 1, 2, or 3; and when n is 1, L is —NHC(O)—, and when n is 2 or 3, L is —C(O)NH—, —NHC(O)—, or —NHC(O)NH; and X is N, or CH.

Pharmaceutical compositions comprising such compounds, and methods of using such compounds in treating conditions in which PDGFR signaling is implicated are also provided.

### DETAILED DESCRIPTION OF ILLUSTRATIVE EMBODIMENTS

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (e.g., to at least one) of the grammatical object of the article unless the context clearly indicates otherwise. By way of example, “an element” means one element or more than one element.

The term “compound”, as used herein, unless otherwise indicated, refers to any specific chemical compound disclosed herein and includes tautomers, optical isomers (enantiomers) and other stereoisomers (diastereomers) thereof, as well as pharmaceutically acceptable salts and derivatives, including prodrug and/or deuterated forms thereof where applicable. Deuterated small molecules contemplated are those in which one or more of the hydrogen atoms contained in the drug molecule have been replaced by deuterium. It is understood by those of ordinary skill that molecules which are described herein are stable compounds as generally described hereunder.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith.

A “solvate” refers to a physical association of a compound of formula (I) or formula (Io) with one or more solvent molecules.

The term “alkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group. Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. In some embodiments, the alkyl group is a C₁-C₆ alkyl; in some embodiments, it is a C₁-C₄ alkyl.

When a range of carbon atoms is used herein, for example, C₁-C₆, all ranges, as well as individual numbers of carbon atoms are encompassed. For example, “C₁-C₃” includes C₁-C₃, C₁-C₂, C₂-C₃, C₁, C₂, and C₃.

The term “cycloalkyl” when used alone or as part of a substituent group refers to cyclic-containing, non-aromatic hydrocarbon groups having from 3 to 10 carbon atoms (“C₃-C₁₀”), preferably from 3 to 6 carbon atoms (“C₃-C₆”). Examples of cycloalkyl groups include cyclopropyl, cyclobutyl, cyclopentyl, cyclohexyl, cycloheptyl, cyclooctyl, indenyl, bicyclo[2.2.1]heptanyl, bicyclo[3.1.1]heptanyl, bicyclo[4.1.0]heptanyl, spiro[3.3]heptanyl, and spiro[3.4]octanyl.

The term “fluoroalkyl” when used alone or as part of a substituent group refers to an alkyl group wherein one or more of the hydrogen atoms has been replaced with one or more fluorine atoms. Examples of fluoroalkyl groups include —CF₃, CHF₂, —CH₂F and the like.

The term “heterocycloalkyl” when used alone or as part of a substituent group refers to any three to twelve-membered monocyclic, saturated or partially unsaturated ring containing at least one heteroatom that is O, N or S. The heterocycloalkyl group may be attached at any heteroatom or carbon atom of the ring such that the result is a stable structure. Examples of heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, tetrahydrofuranyl, tetrahydropyranyl, and the like.

The term “bridged heterocycloalkyl ring” refers to any 5 to 12 membered heterocycloalkyl ring system that contains at least one bridged ring. Examples of bridged heterocycloalkyl rings include azabicyclo[3.1.1]heptane, azabicyclo[3.1.1]heptane, azabicyclo[2.2.2]octane, azabicyclo[2.2.1]heptane, azabicyclo[2.1.1]hexane, azabicyclo[1.1.1]pentane, azabicyclo[1.1.1]pentane, 6-oxa-azabicyclo[3.1.1]heptane, 6-diazabicyclo[3.1.1]heptane, 3-thia-azabicyclo[3.1.1]heptane, and the like.

The term “fused heterocycloalkyl ring system” refers to a heterocycloalkyl ring to which another ring is fused. The other ring that is fused to the heterocycle ring may be another heterocycloalkyl ring, a cycloalkyl ring, an aryl ring, or a heteroaryl ring. In some embodiments, the fused heterocycloalkyl ring system is a 4 to 12 membered fused heterocycloalkyl ring system.

The term “spiroheterocycloalkyl ring system” refers to a heterocycloalkyl ring that is substituted with a spirocyclic ring. The spirocyclic ring can be a cycloalkyl ring of a heterocycloalkyl ring. In some embodiments, the spiroheterocycloalkyl ring system is a 5-12-membered spiroheterocycloalkyl ring system.

The terms “halo” or “halogen”, by itself or as part of another substituent, means a fluorine, chlorine, bromine, or iodine atom.

The term “aryl” when used alone or as part of a substituent group also refers to a mono- or bicyclic-aromatic hydrocarbon ring structure having 6 or 10 carbon atoms in the ring, wherein one or more of the carbon atoms in the ring is optionally substituted. The term “aryl” also includes a mono- or bicyclic-aromatic hydrocarbon ring structure having 6 or 10 carbon atoms in the ring, wherein two adjacent carbon atoms in the ring are optionally substituted such that said two adjacent carbon atoms and their respective substituents form a cycloalkyl or heterocycloalkyl ring. Non-limiting examples of aryl groups include, but are not limited to, phenyl, indenyl, naphthyl, 1, 2, 3,4-tetrahydronaphthyl, and the like.

The term “heteroaryl” when used alone or as part of a substituent group refers to a mono- or bicyclic-aromatic ring structure including carbon atoms as well as up to four heteroatoms that are each independently nitrogen, oxygen, or sulfur. Heteroaryl rings can include a total of 5, 6, 9, or 10 ring atoms. The heteroaryl moiety can be unsubstituted, or one or more of the carbon atoms in the ring can be substituted. Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl, benzofuranyl, benzimidazolyl, thianaphthenyl, pyrrolo[2,3-b]pyridinyl, quinazolinyl-4 (3H)-one, triazolyl, 4,5,6,7-tetrahydro-1H-indazole.

The term “optionally substituted,” as used herein to describe a substituent defined herein, means that the substituent may, but is not required to be, substituted with one or more suitable functional groups or other substituents as provided herein. For example, a substituent may be optionally substituted with one or more of: halo (i.e., —F, —Cl, —Br, —I), cyano, —OH, —C₁-C₆alkyl, C₃-C₆cycloalkyl, C₂-C₆alkenyl, C₂-C₆ alkynyl, C₁-C₆ haloalkyl, —C₁-C₆ alkoxy, —C₁-C₆ haloalkoxy, C₁-C₆ alkylthio, C₁-C₆ alkylamino, —NH₂, —NH(C₁-C₆ alkyl), —N(C₁-C₆ alkyl)₂, —NH(C₁-C₆ alkoxy), —C(O)NHC₁-C₆ alkyl, —C(O)N(C₁-C₆ alkyl)₂, —COOH, —C₁-C₆alkylCOOH, C₃-C₆cycloalkylCOOH, —C(O)NH₂, C₁-C₆alkylCONH₂, —C₃-C₆cycloalkylCONH₂, C₁-C₆alkylCONHC₁-C₆alkyl, C₁-C₆alkylCON(C₁-C₆alkyl)₂, —C(O)C₁-C₆ alkyl, —C(O)OC₁-C₆ alkyl, —NHCO(C₁-C₆ alkyl), —N(C₁-C₆ alkyl)C(O)(C₁-C₆ alkyl), —S(O)C₁-C₆ alkyl, —S(O)₂C₁-C₆ alkyl, oxo, 6-12 membered aryl, or 5 to 12 membered heteroaryl groups.

In particular, a substituent may be optionally substituted with one or more of: halo (i.e., —F, —Cl, —Br, —I), cyano, —C₁-C₆alkyl, C₃-C₆cycloalkyl, C₂-C₆alkenyl, C₂-C₆ alkynyl, C₁-C₆ haloalkyl, —C₁-C₆ alkoxy, —C₁-C₆ haloalkoxy, C₁-C₆ alkylthio, C₁-C₆ alkylamino, —NH₂, —NH(C₁-C₆ alkyl), —N(C₁-C₆ alkyl)₂, —NH(C₁-C₆ alkoxy), —C(O)NHC₁-C₆ alkyl, —C(O)N(C₁-C₆ alkyl)₂, —COOH, —C₁-C₆alkylCOOH, —C₃-C₆cycloalkylCOOH, —C(O)NH₂, C₁-C₆alkylCONH₂, —C₃-C₆cycloalkylCONH₂, C₁-C₆alkylCONHC₁-C₆alkyl, C₁-C₆alkylCON(C₁-C₆alkyl)₂, —C(O)C₁-C₆ alkyl, —C(O)OC₁-C₆ alkyl, —NHCO(C₁-C₆ alkyl), —N(C₁-C₆ alkyl)C(O)(C₁-C₆ alkyl), —S(O)C₁-C₆ alkyl, —S(O)₂C₁-C₆ alkyl, oxo, 6-12 membered aryl, or 5 to 12 membered heteroaryl groups. In some embodiments, each of the above optional substituents are themselves optionally substituted by one or two groups.

In other embodiments, a substituent may be optionally substituted with one or more of: halo (i.e., —F, —Cl, —Br, —I), cyano, —OH, —C₁-C₆alkyl, —CH₂CH₂OH, —CH₂CH₂CH(OH)CH₂(OH), —CH₂CH(OH)CH₂(OH), —CH₂CH(OH)CH₃, —CH₂OH, —C(CH₃)₂CH₂(OH), —CH₂OCH₃, —CH₂CH₂OCH₃, —CH₂—(C₃-C₆cycloalkyl), —C₃-C₆cycloalkyl, C₂-C₆alkenyl, C₂-C₆ alkynyl, —C₁-C₆ haloalkyl, —C₁-C₆ alkoxy, —OCH₃, —C₁-C₆ haloalkoxy, —OCH₂CH₂F, —C₁-C₆ alkylthio, C₁-C₆ alkylamino, —NH₂, —NH(C₁-C₆ alkyl), —N(C₁-C₆ alkyl)₂, —NH(C₁-C₆ alkoxy), —C(O)NHC₁-C₆ alkyl, —CH₂C(O)NHC₁-C₆ alkyl, —C(O)N(C₁-C₆ alkyl)₂, —COOH, —C₁-C₆alkylCOOH, C₃-C₆cycloalkylCOOH, —C(O)NH₂, —C₁-C₆alkylCONH₂, —C₁. C₆alkyl-CN, —C₃-C₆cycloalkylCONH₂, —C₁-C₆alkylCONHC₁-C₆alkyl, C₁-C₆alkylCON(C₁-C₆alkyl)₂, —C(O)C₁-C₆ alkyl, —C(O)OC₁-C₆ alkyl, —NHCO(C₁-C₆ alkyl), —N(C₁-C₆ alkyl)C(O)(C₁-C₆ alkyl), —S(O)C₁-C₆ alkyl, —S(O)₂C₁-C₆ alkyl, —C₁-C₆ alkyl-S(O)₂C₁-C₆ alkyl, oxo, a 4-7 membered heterocycloalkyl group, —CH₂-(4-7 membered heterocycloalkyl), 6-12 membered aryl, 5 to 12 membered heteroaryl groups, —CH₂-(5 to 12 membered heteroaryl)-O—CH₂-(6-12 membered aryl), —CH₂-(5 to 12 membered heteroaryl)-OH. In some embodiments, each of the above optional substituents are themselves optionally substituted by one or two groups.

As used herein, the term “alkenyl” refers to a straight- or branched-chain group having from 2 to 12 carbon atoms (“C₂-C₁₂”), preferably 2 to 4 carbons atoms (“C₂-C₄”), in the group, wherein the group includes at least one carbon-carbon double bond. Examples of alkenyl groups include vinyl (—CH═CH₂; C₂alkenyl) allyl (—CH₂— CH═CH₂; C₃alkenyl), propenyl (—CH═CHCH₃; C₃alkenyl); isopropenyl (—C(CH₃)═CH₂; C₃alkenyl), butenyl (—CH═CHCH₂CH₃; C₄alkenyl), sec-butenyl (—C(CH₃)═CHCH₃; C₄alkenyl), iso-butenyl (—CH═C(CH₃)₂; C₄alkenyl), 2-butenyl (—CH₂CH═CHCH₃; C₄alkyl), pentenyl (—CH═CHCH₂CH₂CH₃; C₅alkenyl), and the like.

As used herein, the term “alkynyl” refers to a straight- or branched-chain group having from 1 to 12 carbon atoms (“C₁-C₁₂”), preferably 1 to 4 carbons atoms (“C₂-C₄”), in the group, and wherein the group includes at least one carbon-carbon triple bond. Examples of alkynyl groups include ethynyl (—C≡CH; C₂alkynyl); propargyl (—CH₂—C≡CH; C₃alkynyl), propynyl (—C≡CCH₃; C₃alkynyl); butynyl (—C≡CCH₂CH₃; C₄alkynyl), pentynyl (—C≡CCH₂CH₂CH₃; C₅alkynyl), and the like.

As used herein, the term “alkoxy” refers to an oxygen radical attached to an alkyl group by a single bond. Examples of alkoxy groups include methoxy (—OCH₃), ethoxy (—OCH₂CH₃), isopropoxy (—OCH(CH₃)₂) and the like.

As used herein, the term “haloalkoxy” refers to an oxygen radical attached to a haloalkyl group by a single bond. Examples of haloalkoxy groups include —OCF₃, —OCH₂CF₃, —OCH(CF₃)₂, and the like.

The term “haloalkyl” refers to an alkyl group wherein one or more of the hydrogen atoms has been replaced with one or more halogen atoms.

The term “haloalkoxy” refers to an alkoxy group wherein one or more of the hydrogen atoms has been replaced with one or more halogen atoms.

As used herein, the term “stereoisomers” refers to compounds which have identical chemical constitution, but differ with regard to the arrangement of the atoms or groups in space, e.g., enantiomers, diastereomers or tautomers.

The term “patient” or “subject” is used throughout the specification to describe an animal, preferably a human or a domesticated animal, to whom treatment, including prophylactic treatment, with the compositions according to the present disclosure is provided. For treatment of those conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

“Treating” or “treatment” of any disease or disorder refers, in one embodiment, to ameliorating the disease or disorder (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

In some aspects, the present disclosure provides compounds of formula (I₀):

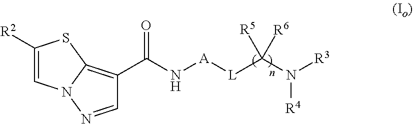

or pharmaceutically acceptable salts thereof, wherein
A is an optionally substituted phenyl ring, an optionally substituted pyridinyl ring, or an optionally substituted 5-membered heteroaryl ring containing 1-2 heteroatoms that are each independently O, N, or S;
R² is optionally substituted aryl, optionally substituted heteroaryl, optional substituted fused heterocycloalkyl, optionally substituted alkyl, optionally substituted alkenyl, optionally substituted cycloalkyl, or optionally substituted heterocycloalkyl;
R³ and R⁴ are each independently optionally substituted aryl, optionally substituted heteroaryl, optionally substituted alkyl, optionally substituted cycloalkyl, or optionally substituted heterocycloalkyl; or one of R³ or R⁴ may be H; or
   R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N;
each R⁵ and each R⁶ is independently H, C₁-C₆alkyl, or C₃-C₅cycloalkyl; or
   an R⁵ and R⁶ attached to the same carbon atom, together with that carbon atom, may form a C₃-C₆cycloalkyl ring; or
   an R⁵ and R⁶ attached to the same carbon atom, together with that carbon atom, may form a C═O; or
   an R⁵ or R⁶, together with an R³ or R⁴ may form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system;
n is 1, 2, 3, 4, or 5; and
L is —NHC(O)— or

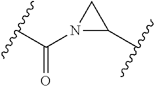 [SMILES: *C(=O)N1CC1*]

when n is 1; or —NHC(O)—, —NHS(O)₂—,

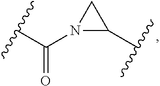 [SMILES: *C(=O)N1CC1*]

-NHC(O)O—, —S(O)₂NH—, —C(O)NH—, or —NHC(O)NH when n is 2,3,4, or 5.

In some aspects, the compounds of formula (Io) are compounds of formula (I):

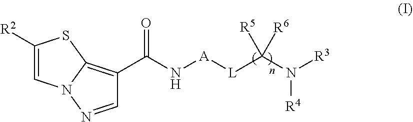

or pharmaceutically acceptable salts thereof, wherein A is an optionally substituted phenyl ring, an optionally substituted pyridinyl ring, or an optionally substituted 5-membered heteroaryl ring containing 1-2 heteroatoms that are each independently O, N, or S; R² is optionally substituted aryl, optionally substituted heteroaryl, optionally substituted alkyl, optionally substituted cycloalkyl, or optionally substituted heterocycloalkyl; R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; n is 1, 2, or 3; and when n is 1, L is —NHC(O)—, and when n is 2 or 3, L is —C(O)NH—, —NHC(O)—, or —NHC(O)NH.

In some aspects, A in the compounds of formula (Io) or the compounds of formula (I) is an optionally substituted phenyl ring, an optionally substituted pyridinyl ring, or an optionally substituted 5-membered heteroaryl ring containing 1-2 heteroatoms that are each independently O, N, or S.

In some embodiments, A in formula (Io) or formula (I) is an optionally substituted phenyl ring.

In other embodiments, A in formula (Io) or formula (I) is an optionally substituted pyridinyl ring.

In some embodiments, A in formula (Io) or formula (I) is an optionally substituted 5-membered heteroaryl ring containing 1-2 heteroatoms that are each independently O, N, or S, such as, for example, furan, pyrrole, thiophene, isoxazole, oxazole, pyrazole, imidazole, isothiazole, thiazole, and the like.

In some embodiments, A in formula (Io) or formula (I) is an optionally substituted thiophene.

In some aspects, R² in the compounds of formula (Io) or the compounds of formula (I) is optionally substituted aryl, optionally substituted heteroaryl, optionally substituted alkyl, optionally substituted cycloalkyl, or optionally substituted heterocycloalkyl.

In some embodiments, R² in the compounds of formula (Io) or the compounds of formula (I) is optionally substituted aryl, such as, for example, phenyl, indenyl, naphthyl, 1, 2, 3,4-tetrahydronaphthyl, and the like.

In some embodiments, R² is optionally substituted phenyl.

In some embodiments of R², the optionally substituted phenyl is (3-hydroxy-oxetan-3-yl)-phen-4-yl.

In some embodiments of R², the optionally substituted phenyl is 1-carboxy-phen-4-yl.

In some embodiments of R², the optionally substituted phenyl is 1-carboxy-phen-3-yl.

In some embodiments, R² in the compounds of formula (Io) or the compounds of formula (I) is optionally substituted heteroaryl, such as, for example, an optionally substituted pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl, benzofuranyl, benzimidazolyl, thianaphthenyl, pyrrolo[2,3-b]pyridinyl, quinazolinyl-4 (3H)-one, triazolyl, 4,5,6,7-tetrahydro-1H-indazole, or 6,7-dihydro-5H-pyrazolo[5,1-b][1,3]oxazine, and in particular an optionally substituted pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl, benzofuranyl, benzimidazolyl, thianaphthenyl, pyrrolo[2,3-b]pyridinyl, quinazolinyl-4 (3H)-one, triazolyl, or 4,5,6,7-tetrahydro-1H-indazole.

In some embodiments of R², the optionally substituted heteroaryl is benzo[d]oxazol-2 (3H)-one-5-yl.

In other embodiments, R² in the compounds of formula (Io) or the compounds of formula (I) is 5,6-dihydro-4H-pyrrolo[1,2-b]pyrazol-3-yl.

In some embodiments of R², the optionally substituted heteroaryl is substituted with an optionally substituted C₁-C₆alkyl, such as, for example, C₁-C₆alkyl, C₁-C₅alkyl, C₁-C₄alkyl, C₁-C₃alkyl, C₁-C₂alkyl, C₆alkyl, C₅alkyl, C₄alkyl, C₃alkyl, C₂alkyl, C₅ alkyl, methyl, ethyl, n-propyl, iso-propyl, n-butyl, t-butyl, iso-butyl, sec-butyl, n-pentyl, n-hexyl, and the like; or an optionally substituted C₃-C₅cycloalkyl, such as, for example, C₃cycloalkyl, C₄cycloalkyl, C₅cycloalkyl, cyclopropyl, cyclobutyl, cyclopentyl, and the like.

In some embodiments of R², the optionally substituted heteroaryl is an optionally substituted 5-membered heteroaryl.

In some embodiments of R², the optionally substituted 5-membered heteroaryl is substituted with an optionally substituted C₁-C₆alkyl, such as, for example, C₁-C₆alkyl, C₁-C₅alkyl, C₁-C₄alkyl, C₁-C₃alkyl, C₁-C₂alkyl, C₆alkyl, C₅alkyl, C₄alkyl, C₃alkyl, C₂alkyl, C₅ alkyl, methyl, ethyl, n-propyl, iso-propyl, n-butyl, t-butyl, iso-butyl, sec-butyl, n-pentyl, n-hexyl, and the like; or an optionally substituted C₃-C₅cycloalkyl, such as, for example, C₃cycloalkyl, C₄cycloalkyl, C₅cycloalkyl, cyclopropyl, cyclobutyl, cyclopentyl, and the like.

In some embodiments of R², the optionally substituted 5-membered heteroaryl is an is an optionally substituted pyrrolyl.

In some embodiments of R², the optionally substituted pyrrolyl is an unsubstituted pyrrolyl.

In some embodiments of R², unsubstituted pyrrolyl is pyrrol-3-yl.

In some embodiments of R², the optionally substituted pyrrolyl is 1-(methylsulfonyl)-1H-pyrrol-3-yl.

In some embodiments of R², the optionally substituted 5-membered heteroaryl is an is an optionally substituted pyrazolyl.

In some embodiments of R², the optionally substituted pyrazolyl is an unsubstituted pyrazolyl.

In some embodiments of R², the unsubstituted pyrazolyl is pyrazol-3-yl.

In some embodiments of R², the unsubstituted pyrazolyl is pyrazol-4-yl.

In some embodiments of R², the optionally substituted pyrazolyl is substituted with an optionally substituted C₁-C₆alkyl, such as, for example, C₁-C₆alkyl, C₁-C₅alkyl, C₁-C₄alkyl, C₁-C₃alkyl, C₁-C₂alkyl, C₆alkyl, C₅alkyl, C₄alkyl, C₃alkyl, C₂alkyl, C₅ alkyl, methyl, ethyl, n-propyl, iso-propyl, n-butyl, t-butyl, iso-butyl, sec-butyl, n-pentyl, n-hexyl, and the like; or an optionally substituted C₃-C₅cycloalkyl, such as, for example, C₃cycloalkyl, C₄cycloalkyl, C₅cycloalkyl, cyclopropyl, cyclobutyl, cyclopentyl, and the like.

In some embodiments of R², the optionally substituted pyrazolyl is substituted with a methyl group, i.e., —CH₃.

In some embodiments of R², the optionally substituted pyrazolyl is substituted with a 2-hydroxyethyl group, i.e., —CH₂CH₂OH.

In some embodiments of R², the optionally substituted pyrazolyl is substituted with a 2-(C₁-C₆alkoxy)ethyl group, i.e., —CH₂CH₂O(C₁-C₆alkyl).

In some embodiments of R², the optionally substituted pyrazolyl is substituted with a 2-methoxyethyl group, i.e., —CH₂CH₂OCH₃.

In other embodiments of R², the optionally substituted pyrazolyl is substituted with a cyclopropyl group.

In some embodiments of R², the optionally substituted pyrazolyl is 1-methyl-pyrazol-3-yl, 1-methyl-pyrazol-4-yl, 1-methyl-pyrazol-5-yl, 1-cyclopropyl-pyrazol-3-yl, 1-cyclopropyl-pyrazol-4-yl, 1-cyclopropyl-pyrazol-5-yl, 1-(2-hydroxyethyl)-pyrazol-3-yl, 1-(2-hydroxyethyl)-pyrazol-4-yl, 1-(2-hydroxyethyl)-pyrazol-5-yl, or 1-(2-methoxyethyl)-1H-pyrazol-4-yl, and in particular optionally substituted pyrazolyl is 1-methyl-pyrazol-3-yl, 1-methyl-pyrazol-4-yl, 1-methyl-pyrazol-5-yl, 1-cyclopropyl-pyrazol-3-yl, 1-cyclopropyl-pyrazol-4-yl, 1-cyclopropyl-pyrazol-5-yl, 1-(2-hydroxyethyl)-pyrazol-3-yl, 1-(2-hydroxyethyl)-pyrazol-4-yl, 1-(2-hydroxyethyl)-pyrazol-5-yl.

In some embodiments of R², the optionally substituted pyrazolyl is 3-methylpyrazol-4-yl.

In some embodiments of R², the optionally substituted pyrazolyl is 1-ethylpyrazol-5-yl.

In some embodiments of R², the optionally substituted pyrazolyl is 1-(cyclopropylmethyl)pyrazol-4-yl.

In some embodiments of R², the optionally substituted pyrazolyl is 1-cyclobutanyl-pyrazol-4-yl.

In other embodiments of R², the optionally substituted pyrazolyl is substituted with two or three methyl groups.

In other embodiments of R², the optionally substituted pyrazolyl is 1,5-dimethyl-pyrazol-4-yl, 1,3-dimethyl-pyrazol-4-yl, 1-(2-methoxyethyl)-3,5-dimethyl-pyrazol-4-yl, 3,5-dimethyl-pyrazol-4-yl, 1,4-dimethyl-pyrazol-5-yl, or 1,3,5-trimethyl-pyrazol-4-yl.

In other embodiments of R², the optionally substituted pyrazolyl is 1-methyl-3-trifluoromethyl-pyrazol-4-yl.

In other embodiments of R², the optionally substituted pyrazolyl is 1-trifluoromethyl-pyrazol-4-yl.

In other embodiments of R², the optionally substituted pyrazolyl is 1-(2,2,2-trifluoroeth-1-yl)-pyrazol-4-yl.

In some embodiments of R², the optionally substituted pyrazolyl is 1-difluoromethylpyrazol-4-yl.

In other embodiments of R², the optionally substituted pyrazolyl is 3,5-dimethyl-1-(2-methoxyethyl)-pyrazol-4-yl.

In other embodiments of R², the optionally substituted pyrazolyl is 3,5-dimethyl-1-(oxetan-3-yl)-1H-pyrazol-4-yl.

In other embodiments of R², the optionally substituted pyrazolyl is 1-(thietan-3-yl 1,1-dioxide)-pyrazol-4-yl, i.e.,

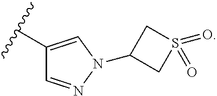 [SMILES: *c1cnn(C2CS(=O)(=O)C2)c1]

In other embodiments of R², the optionally substituted pyrazolyl is 1-(oxetan-3-yl)-1H-pyrazol-4-yl.

In other embodiments of R², the optionally substituted pyrazolyl is 1-(oxetan-3-yl-methyl)-pyrazol-4yl.

In other embodiments of R², the optionally substituted pyrazolyl is 1-((methylsulfonyl)methyl)-pyrazol-4-yl.

In other embodiments of R², the optionally substituted pyrazolyl is 1-((cyano)methyl)-pyrazol-4-yl.

In other embodiments of R², the optionally substituted pyrazolyl is 1-(1-(cyano)eth-1-yl)-pyrazol-4-yl.

In other embodiments of R², the optionally substituted pyrazolyl is (1-hydroxy-2-methylpropan-2-yl)-1H-pyrazol-4-yl.

In other embodiments of R², the optionally substituted pyrazolyl is 1-(acetamid-2-yl)-pyrazol-4-yl.

In other embodiments of R², the optionally substituted pyrazolyl is 1-(N-methylacetamid-2-yl)-pyrazol-4-yl.

In other embodiments of R², the optionally substituted pyrazolyl is 1-(4-piperidinyl)-pyrazol-4-yl.

In other embodiments of R², the optionally substituted pyrazolyl is 1-(2-(methylsulfonyl)ethyl)-1H-pyrazol-4-yl.

In other embodiments of R², the optionally substituted pyrazolyl is 1-(2,3-dihydroxy-propan-1-yl)-pyrazol-3-yl.

In other embodiments of R², the optionally substituted pyrazolyl is 1-(2,3-dihydroxy-propan-1-yl)-pyrazol-4-yl.

In other embodiments of R², the optionally substituted pyrazolyl is 1-(2-hydroxy-propan-1-yl)-pyrazol-4-yl.

In other embodiments of R², the optionally substituted pyrazolyl is 1-(3,4-dihydroxy-butan-1yl)-pyrazol-4-yl.

In other embodiments of R², the optionally substituted pyrazolyl is 1-((3-hydroxy-isoxazol-5-yl)methyl)-pyrazol-4-yl.

In other embodiments of R², the optionally substituted pyrazolyl is 1-((3-benzyloxy-isoxazol-5-yl)methyl)-pyrazol-4-yl.

In other embodiments of R², the optionally substituted pyrazolyl is 1-(pyridin-3-yl)-pyrazol-4-yl.

In other embodiments of R², the optionally substituted pyrazolyl is 3-(hydroxymethyl)-1-methyl-pyrazol-4-yl.

In other embodiments of R², the optionally substituted pyrazolyl is 1-(1-hydroxy-2-methylpropan-2-yl)-pyrazol-4-yl.

In other embodiments of R², the optionally substituted pyrazolyl is 1-(4-tetrahydro-2H-thiopyran 1,1-dioxide)-pyrazol-4-yl, i.e.,

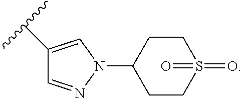

In some embodiments of R², the optionally substituted 5-membered heteroaryl is an is an optionally substituted triazolyl.

In some embodiments of R², the optionally substituted triazolyl is substituted with an optionally substituted C₁-C₆alkyl, such as, for example, C₁-C₆alkyl, C₁-C₅alkyl, C₁-C₄alkyl, C₁-C₃alkyl, C₁-C₂alkyl, C₆alkyl, C₅alkyl, C₄alkyl, C₃alkyl, C₂alkyl, C₅ alkyl, methyl, ethyl, n-propyl, iso-propyl, n-butyl, t-butyl, iso-butyl, sec-butyl, n-pentyl, n-hexyl, and the like; or an optionally substituted C₃-C₅cycloalkyl, such as, for example, C₃cycloalkyl, C₄cycloalkyl, C₅cycloalkyl, cyclopropyl, cyclobutyl, cyclopentyl, and the like.

In some embodiments of R², the optionally substituted 5-membered heteroaryl is 2,4-dimethyl-1,2,3-triazol-5-yl.

In some embodiments of R², the optionally substituted 5-membered heteroaryl is 2-methyl-1,2,3-triazol-4-yl.

In some embodiments of R², the optionally substituted 5-membered heteroaryl is an optionally substituted imidazolyl.

In some embodiments of R², the optionally substituted imidazolyl is 1-methyl-imidazol-4-yl.

In some embodiments of R², the optionally substituted 5-membered heteroaryl is an optionally substituted isoxazolyl.

In some embodiments of R², the optionally substituted isoxazolyl is 3,5-dimethyl-isoxazol-4-yl.

In some embodiments of R², the optionally substituted 5-membered heteroaryl is an optionally substituted furanyl.

In some embodiments of R², the optionally substituted furanyl is 2-(hydroxymethyl)-furan-5-yl.

In some embodiments of R², the optionally substituted 5-membered heteroaryl is furan-3-yl.

In some embodiments of R², the optionally substituted 5-membered heteroaryl is an optionally substituted thiophenyl.

In some embodiments of R², the optionally substituted thiophenyl is thiopheny-3-yl.

In some embodiments of R², the optionally substituted thiophenyl is 2-hydroxymethyl-thiophen-5-yl.

In some embodiments, R² in the compounds of formula (Io) or the compounds of formula (I) is an optionally substituted 6-membered heteroaryl.

In some embodiments of R², the optionally substituted 6-membered heteroaryl is an optionally substituted pyridinyl, an optionally substituted pyridazinyl, or an optionally substituted pyrimidinyl.

In some embodiments of R², the optionally substituted 6-membered heteroaryl is substituted with an optionally substituted C₁-C₆alkyl, such as, for example, C₁-C₆alkyl, C₁-C₅alkyl, C₁-C₄alkyl, C₁-C₃alkyl, C₁-C₂alkyl, C₆alkyl, C₅alkyl, C₄alkyl, C₃alkyl, C₂alkyl, C₅ alkyl, methyl, ethyl, n-propyl, iso-propyl, n-butyl, t-butyl, iso-butyl, sec-butyl, n-pentyl, n-hexyl, and the like; or an optionally substituted C₃-C₅cycloalkyl, such as, for example, C₃cycloalkyl, C₄cycloalkyl, C₅cycloalkyl, cyclopropyl, cyclobutyl, cyclopentyl, and the like.

In some embodiments of R², the optionally substituted 6-membered heteroaryl is an is an optionally substituted pyridinyl.

In some embodiments of R², the optionally substituted pyridinyl is substituted with an optionally substituted C₁-C₆alkyl, such as, for example, C₁-C₆alkyl, C₁-C₅alkyl, C₁-C₄alkyl, C₁-C₃alkyl, C₁-C₂alkyl, C₆alkyl, C₅alkyl, C₄alkyl, C₃alkyl, C₂alkyl, C₅ alkyl, methyl, ethyl, n-propyl, iso-propyl, n-butyl, t-butyl, iso-butyl, sec-butyl, n-pentyl, n-hexyl, and the like; or an optionally substituted C₃-C₅cycloalkyl, such as, for example, C₃cycloalkyl, C₄cycloalkyl, C₅cycloalkyl, cyclopropyl, cyclobutyl, cyclopentyl, and the like.

In some embodiments of R², the optionally substituted pyridinyl is unsubstituted pyridinyl.

In some embodiments of R², the unsubstituted pyridinyl is pyridine-2-yl.

In some embodiments of R², the unsubstituted pyridinyl is pyridine-3-yl.

In some embodiments of R², the unsubstituted pyridinyl is pyridine-4-yl.

In some embodiments of R², the optionally substituted pyridinyl is C₁-C₆alkoxyl substituted pyridinyl.

In some embodiments of R², the optionally substituted pyridinyl is methoxy substituted pyridinyl, such as, for example, 4-methoxypyridin-3-yl.

In some embodiments of R², the optionally substituted pyridinyl is 2-methoxypyridin-3-yl.

In some embodiments of R², the optionally substituted pyridinyl is 2-methoxypyridin-5-yl.

In some embodiments of R², the optionally substituted pyridinyl is 2-methoxypyridin-6-yl.

In some embodiments of R², the optionally substituted pyridinyl is 4-methoxypyridin-3-yl.

In some embodiments of R², the optionally substituted pyridinyl is 3-methoxypyridin-4-yl.

In some embodiments of R², the optionally substituted pyridinyl is 2-ethoxypyridin-3-yl.

In some embodiments of R², the optionally substituted pyridinyl is 2-trifluoromethoxypyridin-3-yl.

In some embodiments of R², the optionally substituted pyridinyl is 2-hydroxypyridin-3-yl.

In some embodiments of R², the optionally substituted pyridinyl is 2-hydroxypyridin-5-yl.

In some embodiments of R², the optionally substituted pyridinyl is 2-methylpyridin-3-yl.

In some embodiments of R², the optionally substituted pyridinyl is 2-methylpyridin-5-yl.

In some embodiments of R², the optionally substituted pyridinyl is 2-ethylpyridin-3-yl.

In some embodiments of R², the optionally substituted pyridinyl is 2-(2-fluoroethoxy)pyridin-3-yl.

In some embodiments of R², the optionally substituted pyridinyl is 2-amino-3-fluoro-pyridin-5-yl.

In some embodiments of R², the optionally substituted pyridinyl is 2-amino-pyridin-5-yl or 6-aminopyridin-3-yl.

In some embodiments of R², the optionally substituted pyridinyl is 2-(4-morpholinyl)-pyridin-4-yl.

In some embodiments of R², the optionally substituted pyridinyl is 2-(dimethylamino)pyridin-4-yl.

In some embodiments of R², the optionally substituted pyridinyl is 3-(methylsulfonyl)pyridin-5-yl.

In some embodiments of R², the optionally substituted pyridinyl is 4-(acetylamino)-pyridin-2-yl.

In some embodiments of R², the optionally substituted pyridinyl is 3-(acetylamino)-pyridin-5-yl.

In some embodiments of R², the optionally substituted pyridinyl is 2-(acetylamino)-pyridin-4-yl.

In some embodiments of R², the optionally substituted pyridinyl is 2-(N-methylacetamid)-pyridin-4-yl, i.e.,

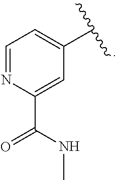 [SMILES: *c1ccnc(C(=O)NC)c1]

In some embodiments of R², the optionally substituted 6-membered heteroaryl is an is an optionally substituted pyridazinyl.

In some embodiments of R², the optionally substituted pyridaziny is 3-methyl-pyridazin-5-yl.

In some embodiments of R², the optionally substituted pyridaziny is 3,6-dimethoxy-pyridazin-4-yl.

In some embodiments of R², the optionally substituted pyridaziny is 3-hydroxy-pyridazin-6-yl.

In some embodiments of R², the optionally substituted 6-membered heteroaryl is an is an optionally substituted pyrimidinyl.

In some embodiments of R², the optionally substituted pyrimidinyl is pyrimidin-5-yl.

In some embodiments of R², the optionally substituted pyrimidinyl is 2-methoxy-4-hydroxy-pyrimidin-5-yl.

In some embodiments of R², the optionally substituted pyrimidinyl is 2,4-dimethoxy-pyrimidin-5-yl.

In some embodiments of R², the optionally substituted pyrimidinyl is 4-methyl-pyrimidin-5-yl.

In some embodiments of R², the optionally substituted heteroaryl is an optionally substituted 6,7-dihydro-5H-pyrazolo[5,1-b][1,3]oxazine.

In some embodiments of R², the optionally substituted 6,7-dihydro-5H-pyrazolo[5,1-b][1,3]oxazine is 6,7-dihydro-5H-pyrazolo[5,1-b][1,3]oxazin-3-yl.

In some embodiments of R², the optionally substituted heteroaryl is 6,7-dihydro-4H-pyrazolo[5,1-c][1,4]oxazin-3-yl.

In some embodiments of R², the optionally substituted heteroaryl is 5,6-dihydro-8H-imidazo[2,3-c][1,4]oxazin-3-yl.

In some embodiments of R², the optionally substituted heteroaryl is 7,8-dihydro-5H-imidazo[3,2-c][1,3]oxazin-3-yl.

In some embodiments of R², the optionally substituted heteroaryl is an optionally substituted 1-methylindazol-4-yl.

In some embodiments of R², the optionally substituted heteroaryl is an optionally substituted 1H-pyrazolo[3,4-b]pyridin-1-yl.

In some embodiments of R², the optionally substituted 1H-pyrazolo[3,4-b]pyridin-1-yl is unsubstituted 1H-pyrazolo[3,4-b]pyridin-1-yl.

In some embodiments of R², the optionally substituted heteroaryl is an optionally substituted 1H-pyrazolo[3,4-b]pyridine-5-yl.

In some embodiments of R², the optionally substituted heteroaryl is an optionally substituted indolyl.

In some embodiments of R², the optionally substituted indolyl is an unsubstituted indolyl.

In some embodiments of R², the unsubstituted indolyl is indol-3-yl.

In some embodiments of R², the optionally substituted heteroaryl is 2-oxo-2,3-dihydrobenzo[d]oxazol-5-yl.

In some embodiments, R² in the compounds of formula (Io) is optionally substituted fused heterocycloalkyl.

In some embodiments, R² in the compounds of formula (Io) or the compounds of formula (I) is optionally substituted alkyl.

In some embodiments, R² in the optionally substituted alkyl is 3-methoxyprop-1-yl.

In some embodiments, R² in the compounds of formula (Io) is optionally substituted alkenyl.

In some embodiments of R² the optionally substituted alkenyl is (E)-3-methoxyprop-1-en-1-yl.

In some embodiments, R² in the compounds of formula (Io) or the compounds of formula (I) is optionally substituted cycloalkyl.

In some embodiments, R² in the compounds of formula (Io) or the compounds of formula (I) is optionally substituted heterocycloalkyl.

In some embodiments of R², the optionally substituted heterocycloalkyl is 1-(2-fluoroethyl)-2-oxo-1,2-dihydropyridin-3-yl.

In some embodiments of R², the optionally substituted heterocycloalkyl is 1-methyl-6-oxo-1,6-dihydropyridin-3-yl.

In some aspects, n in the compounds of formula (Io) is 1, 2, 3, 4, or 5.

In some aspects, n in the compounds of formula (I) is 1, 2, or 3.

In some embodiments, n in the compounds of formula (Io) or the compounds of formula (I) is 1.

In some embodiments, n in the compounds of formula (Io) or the compounds of formula (I) is 2.

In some embodiments, n in the compounds of formula (Io) or the compounds of formula (I) is 3.

In some embodiments, n in the compounds of formula (Io) is 4.

In some embodiments, n in the compounds of formula (Io) is 5.

In some aspects of the compounds of formula (Io), L is —NHC(O)— or

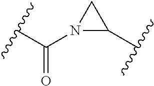 [SMILES: *C(=O)N1CC1*]

when n is 1; or —NHC(O)—, —NHS(O)₂—,

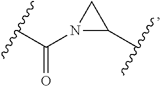 [SMILES: *C(=O)N1CC1*]

—NHC(O)O—, —S(O)₂NH—, —C(O)NH—, or —NHC(O)NH— when n is 2, 3, 4, or 5.

The diradicals “-L-,” as used herein, are written from left-to-right such that the left hand side of L is attached to the moiety A in the compounds of the disclosure.

In some embodiments of the compounds of formula (Io), L is —NHC(O)O—, —S(O)₂NH—, or —NHS(O)₂—.

In some embodiments of the compounds of formula (Io), n is 1 and L is —NHC(O)—.

In some embodiments of the compounds of formula (Io), n is 1 and L is

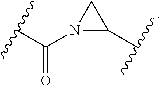 [SMILES: *C(=O)N1CC1*]

In some embodiments of the compounds of formula (Io), n is 2, 3, 4, or 5 and L is —NHC(O)—.

In some embodiments of the compounds of formula (Io), n is 2, 3, 4, or 5 and L is —NHS(O)₂—.

In some embodiments of the compounds of formula (Io), n is 2, 3, 4, or 5 and L is

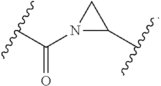 [SMILES: *C(=O)N1CC1*]

In some embodiments of the compounds of formula (Io), n is 2, 3, 4, or 5 and L is —NHC(O)O—.

In some embodiments of the compounds of formula (Io), n is 2, 3, 4, or 5 and L is —S(O)₂NH—.

In some embodiments of the compounds of formula (Io), n is 2, 3, 4, or 5 and L is —C(O)NH—.

In some embodiments of the compounds of formula (Io), n is 2, 3, 4, or 5 and L is —NHC(O)NH.

In some aspects, when n is 1 in the compounds of formula (I), L is —NHC(O)—, and when n is 2 or 3 in the compounds of formula (I), L is —C(O)NH—, —NHC(O)—, or —NHC(O)NH.

In some embodiments of the compounds of formula (I), n is 2 or 3 and L is —C(O)NH—.

In some embodiments of the compounds of formula (I), n is 2 and L is —C(O)NH—.

In some embodiments of the compounds of formula (I), n is 3 and L is —C(O)NH—.

In other embodiments of the compounds of formula (I), n is 1 and L is —NHC(O)—.

In other embodiments of the compounds of formula (I), n is 2 and L is —NHC(O)—.

In other embodiments of the compounds of formula (I), n is 3 and L is —NHC(O)—.

In other embodiments of the compounds of formula (I), n is 2 or 3 and L is —NHC(O)NH—.

In other embodiments of the compounds of formula (I), n is 2 and L is —NHC(O)NH—.

In other embodiments of the compounds of formula (I), n is 3 and L is —NHC(O)NH—.

In some aspects, R³ and R⁴ in the compounds of formula (Io) are each independently optionally substituted aryl, optionally substituted heteroaryl, optionally substituted alkyl, optionally substituted cycloalkyl, or optionally substituted heterocycloalkyl; or one of R³ or R⁴ in the compounds of formula (Io) may be H; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N.

In some embodiments, one of R³ or R⁴ in compounds of formula (Io) is H.

In some aspects, R³ and R⁴ in the compounds of formula (I) are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N.

In some embodiments, R³ or R⁴ in the compounds of formula (Io) or the compounds of formula (I) is optionally substituted aryl, such as, for example, optionally substituted phenyl, indenyl, naphthyl, or 1,2,3,4-tetrahydronaphthyl.

In some embodiments, R³ or R⁴ in the compounds of formula (Io) or the compounds of formula (I) is optionally substituted heteroaryl, such as, for example, optionally substituted pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4 (3H)-one, triazolyl, 4,5,6,7-tetrahydro-1H-indazole.

In some embodiments, R³ or R⁴ in the compounds of formula (Io) or the compounds of formula (I) is optionally substituted alkyl, such as, for example, optionally substituted C₁-C₆alkyl, C₁-C₅alkyl, C₁-C₄alkyl, C₁-C₃alkyl, C₁-C₂alkyl, C₆alkyl, C₅alkyl, C₄alkyl, C₃alkyl, C₂alkyl, C₅ alkyl, methyl, ethyl, n-propyl, iso-propyl, n-butyl, t-butyl, iso-butyl, sec-butyl, n-pentyl, n-hexyl, and the like.

In some embodiments, R³ or R⁴ in the compounds of formula (Io) or the compounds of formula (I) is —CH₃.

In some embodiments, R³ or R⁴ in the compounds of formula (Io) or the compounds of formula (I) is —CH₂CH(CH₃)₂.

In some embodiments, R³ or R⁴ in the compounds of formula (Io) or the compounds of formula (I) is —CH₂CH₂CH₃.

In some embodiments, R³ or R⁴ in the compounds of formula (Io) or the compounds of formula (I) is —CH(CH₃)₂.

In some embodiments, R³ or R⁴ in the compounds of formula (Io) or the compounds of formula (I) is —C(CH₃)₃.

In some embodiments, R³ or R⁴ in the compounds of formula (Io) or the compounds of formula (I) is —CH₂CH₂OCH₃.

In some embodiments, R³ or R⁴ in the compounds of formula (Io) or the compounds of formula (I) is —CH₂CH₂OH.

In some embodiments, R³ or R⁴ in the compounds of formula (Io) or the compounds of formula (I) is —CH₂-cyclohexyl.

In some embodiments, R³ or R⁴ in the compounds of formula (Io) or the compounds of formula (I) is —CH₂-cyclopropyl.

In some embodiments, R³ or R⁴ in the compounds of formula (Io) or the compounds of formula (I) is optionally substituted cycloalkyl, such as, for example, optionally substituted C₃-C₆cycloalkyl, C₃cycloalkyl, C₄cycloalkyl, C₅cycloalkyl, C₆cycloalkyl, cyclopropyl, cyclobutyl, cyclopentyl, cyclohexyl, and the like.

In some embodiments, R³ or R⁴ in the compounds of formula (Io) or the compounds of formula (I) is optionally substituted cyclopentyl.

In some embodiments, R³ or R⁴ in the compounds of formula (Io) or the compounds of formula (I) is unsubstituted cyclopentyl.

In some embodiments, R³ or R⁴ in the compounds of formula (Io) or the compounds of formula (I) is optionally substituted cyclobutyl.

In some embodiments, R³ or R⁴ in the compounds of formula (Io) or the compounds of formula (I) is 1-methyl-cyclobut-1-yl.

In some embodiments, R³ or R⁴ in the compounds of formula (Io) or the compounds of formula (I) is unsubstituted cyclobutyl.

In some embodiments, R³ or R⁴ in the compounds of formula (Io) or the compounds of formula (I) is optionally substituted cyclohexyl.

In some embodiments, R³ or R⁴ in the compounds of formula (Io) or the compounds of formula (I) is unsubstituted cyclohexyl.

In some embodiments, R³ or R⁴ in the compounds of formula (Io) or the compounds of formula (I) is optionally substituted heterocycloalkyl, such as, for example, pyrrolidinyl, tetrahydrofuranyl, tetrahydrothiophenyl, isoxazolidinyl, oxazolidinyl, pyrazolidinyl, imidazolidinyl, or thiazolidinyl.

In some embodiments, R³ or R⁴ in the compounds of formula (Io) or the compounds of formula (I) is tetrahydropyran-4-yl.

In some aspects, R³ and R⁴ in the compounds of formula (Io) or the compounds of formula (I), together with the nitrogen atom to which they are both attached, form an optionally substituted 3-12-membered heterocycloalkyl ring, an optionally substituted 5-12-membered bridged heterocycloalkyl ring; an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, and 5-12-membered spiroheterocycloalkyl ring system may optionally include, in addition to the nitrogen atom to which both R³ and R⁴ are attached, 1-3 heteroatoms that are each independently O, S, or N.

In some aspects, R³ and R⁴ in the compounds of formula (Io) or the compounds of formula (I), together with the nitrogen atom to which they are both attached, form an optionally substituted 3-12-membered heterocycloalkyl ring. Non-limiting examples of such heterocycloalkyl rings include the following:

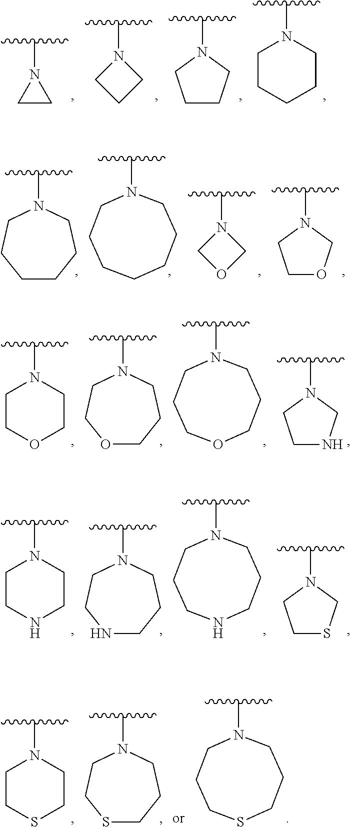

In some embodiments, the optionally substituted 3-12-membered heterocycloalkyl ring is substituted with at least one C₁-C₆alkyl group, such as, for example, C₁-C₆alkyl, C₁-C₅alkyl, C₁-C₄alkyl, C₁-C₃alkyl, C₁-C₂alkyl, C₆alkyl, C₅alkyl, C₄alkyl, C₃alkyl, C₂alkyl, C₅ alkyl, methyl, ethyl, n-propyl, iso-propyl, n-butyl, t-butyl, iso-butyl, sec-butyl, n-pentyl, n-hexyl, and the like.

In some embodiments, the optionally substituted 3-12-membered heterocycloalkyl ring is substituted with at least one —CH₃ group.

In some embodiments, R³ and R⁴, together with the nitrogen atom to which they are both attached, form a 2,2-dimethylpyrrolidin-1-yl group,

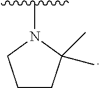 [SMILES: *N1CCCC1(C)C]

In other embodiments, R³ and R⁴, together with the nitrogen atom to which they are both attached, form a 3,3-dimethylpyrrolidin-1-yl group,

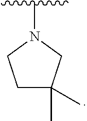 [SMILES: *N1CCC(C)(C)C1]

In other embodiments, R³ and R⁴, together with the nitrogen atom to which they are both attached, form a 3,3-dimethylazetidin-1-yl group,

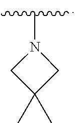 [SMILES: *N1CC(C)(C)C1]

In other embodiments, R³ and R⁴, together with the nitrogen atom to which they are both attached, form a 2-isopropylazetidin-1-yl group,

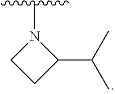 [SMILES: *N1CCC1C(C)C]

In other embodiments, R³ and R⁴, together with the nitrogen atom to which they are both attached, form a 2-methyl-pyrrolidin-1-yl group,

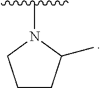 [SMILES: *N1CCCC1C]

In other embodiments, R³ and R⁴, together with the nitrogen atom to which they are both attached, form a (R)-2-methyl-pyrrolidin-1-yl group.

In other embodiments, R³ and R⁴, together with the nitrogen atom to which they are both attached, form a (S)-2-methyl-pyrrolidin-1-yl group.

In some embodiments, R³ and R⁴, together with the nitrogen atom to which they are both attached, form a 2-methyl-piperidin-1-yl group,

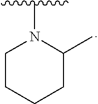 [SMILES: *N1CCCCC1C]

In some embodiments, R³ and R⁴, together with the nitrogen atom to which they are both attached, form a (R)-2-methyl-piperidin-1-yl group.

In other embodiments, R³ and R⁴, together with the nitrogen atom to which they are both attached, form a (S)-2-methyl-piperidin-1-yl group.

In some embodiments, the optionally substituted 3-12-membered heterocycloalkyl ring is substituted with at least one halogen atom. In some embodiments, the halogen atom is —F.

In some embodiments, R³ and R⁴, together with the nitrogen atom to which they are both attached, form a 4,4-difluoropiperidin-1-yl group,

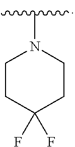 [SMILES: *N1CCC(F)(F)CC1]

In some embodiments, R³ and R⁴, together with the nitrogen atom to which they are both attached, form a 3,3-difluoropiperidin-1-yl group,

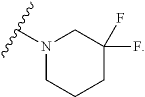 [SMILES: *N1CCCC(F)(F)C1]

In some embodiments, R³ and R⁴, together with the nitrogen atom to which they are both attached, form a 3-methylmorpholino group,

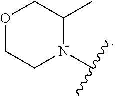 [SMILES: *N1CCOCC1C]

In some embodiments, R³ and R⁴, together with the nitrogen atom to which they are both attached, form a 3(R)-methylmorpholino group.

In some embodiments, R³ and R⁴, together with the nitrogen atom to which they are both attached, form a 3(S)-methylmorpholino group.

In some embodiments, R³ and R⁴, together with the nitrogen atom to which they are both attached, form a 3,5-dimethylmorpholino group,

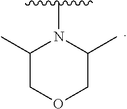

In some embodiments, R³ and R⁴, together with the nitrogen atom to which they are both attached, form a 3(R),5(R)-dimethylmorpholino group.

In some embodiments, R³ and R⁴, together with the nitrogen atom to which they are both attached, form a 3(S),5(S)-dimethylmorpholino group.

In some embodiments, R³ and R⁴, together with the nitrogen atom to which they are both attached, form a 3(R),5(S)-dimethylmorpholino group.

In some embodiments, R³ and R⁴, together with the nitrogen atom to which they are both attached, form a 3(S),5(R)-dimethylmorpholino group.

In some embodiments, R³ and R⁴, together with the nitrogen atom to which they are both attached, form a 2,6-dimethylmorpholino group,

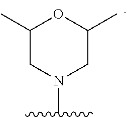

In some embodiments, R³ and R⁴, together with the nitrogen atom to which they are both attached, form a 2(R),6(R)-dimethylmorpholino group.

In some embodiments, R³ and R⁴, together with the nitrogen atom to which they are both attached, form a 2(S),6(S)-dimethylmorpholino group.

In some embodiments, R³ and R⁴, together with the nitrogen atom to which they are both attached, form a 2(R),6(S)-dimethylmorpholino group.

In some embodiments, R³ and R⁴, together with the nitrogen atom to which they are both attached, form a 2(S),6(R)-dimethylmorpholino group.

In some embodiments, R³ and R⁴, together with the nitrogen atom to which they are both attached, form a 3-methylmorpholino group,

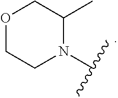 [SMILES: *N1CCOCC1C]

In some embodiments, R³ and R⁴, together with the nitrogen atom to which they are both attached, form a 3(R)-methylmorpholino group.

In some embodiments, R³ and R⁴, together with the nitrogen atom to which they are both attached, form a 3(S)-methylmorpholino group.

In some aspects, R³ and R⁴ in the compounds of formula (Io) or the compounds of formula (I), together with the nitrogen atom to which they are both attached, form an optionally substituted 5-12-membered bridged heterocycloalkyl ring. Non-limiting examples of such bridged heterocycloalkyl ring systems include:

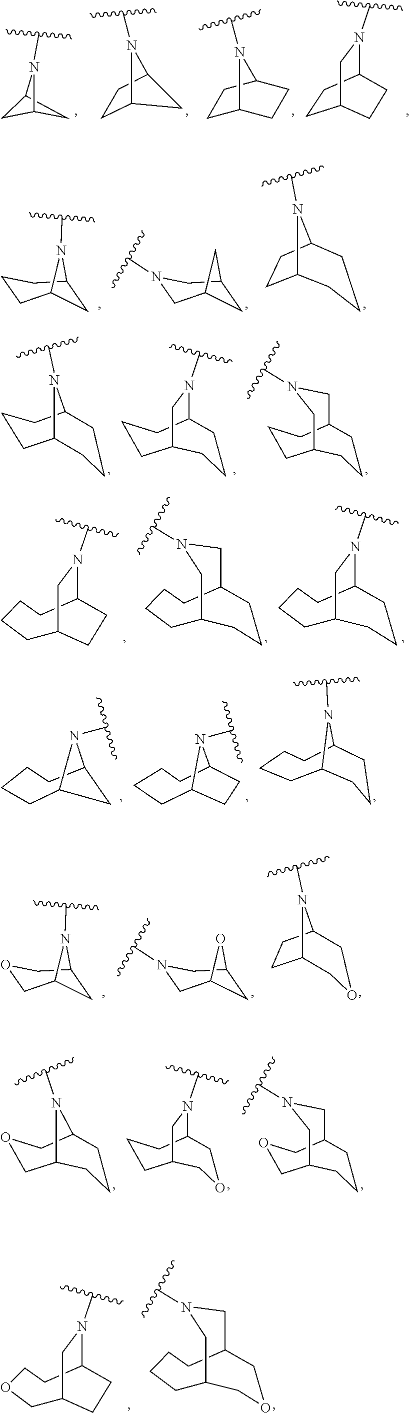

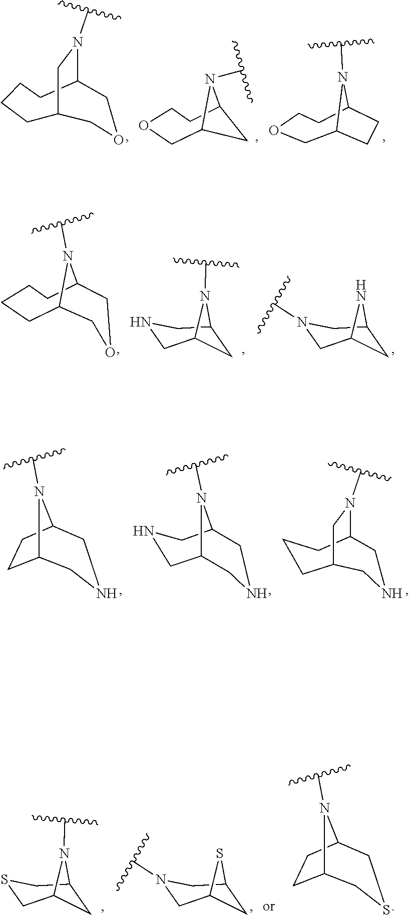

In some embodiments, R³ and R⁴, together with the nitrogen atom to which they are both attached, form a 2-azabicyclo[2.2.2]octan-2-yl group:

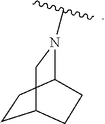 [SMILES: *N1CC2CCC1CC2]

In other embodiments, R³ and R⁴, together with the nitrogen atom to which they are both attached, form an 9-azabicyclo[3.3.1]nonan-9-yl group:

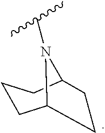 [SMILES: *N1C2CCCC1CC2]

In other embodiments, R³ and R⁴, together with the nitrogen atom to which they are both attached, form an 3-azabicyclo[3.1.1]heptan-3-yl group,

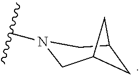 [SMILES: *N1CC2CC(C2)C1]

In other embodiments, R³ and R⁴, together with the nitrogen atom to which they are both attached, form an 3-oxa-8-azabicyclo[3.2.1]octan-8-yl group,

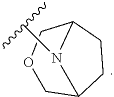 [SMILES: *N1C2CCC1COC2]

In other embodiments, R³ and R⁴, together with the nitrogen atom to which they are both attached, form an 6-oxa-3-azabicyclo[3.1.1]heptan-3-yl group,

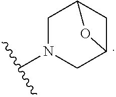 [SMILES: *N1CC2CC(C1)O2]

In other embodiments, R³ and R⁴, together with the nitrogen atom to which they are both attached, form (1S,4R)-2-azabicyclo[2.2.1]heptan-2-yl group,

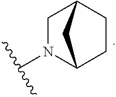 [SMILES: *N1C[C@@H]2CC[C@H]1C2]

In other embodiments, R³ and R⁴, together with the nitrogen atom to which they are both attached, form a (1R,4S)-2-azabicyclo[2.2.1]heptan-2-yl group,

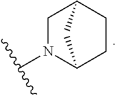 [SMILES: *N1C[C@H]2CC[C@@H]1C2]

In other embodiments, R³ and R⁴, together with the nitrogen atom to which they are both attached, form a 8-oxa-3-azabicyclo[3.2.1]octan-3-yl group,

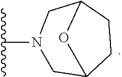 [SMILES: *N1CC2CCC(C1)O2]

In other embodiments, R³ and R⁴, together with the nitrogen atom to which they are both attached, form a 7-azabicyclo[2.2.1]heptan-7-yl group,

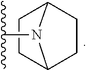 [SMILES: *N1C2CCC1CC2]

In other embodiments, R³ and R⁴, together with the nitrogen atom to which they are both attached, form a 2-azabicyclo[2.2.1]heptan-2-yl group,

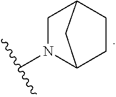 [SMILES: *N1CC2CCC1C2]

In some aspects, R³ and R⁴ in the compounds of formula (I) or formula (Io), together with the nitrogen atom to which they are both attached, form an optionally substituted 5-12-membered spiroheterocycloalkyl ring. Non-limiting examples of such ring systems include:

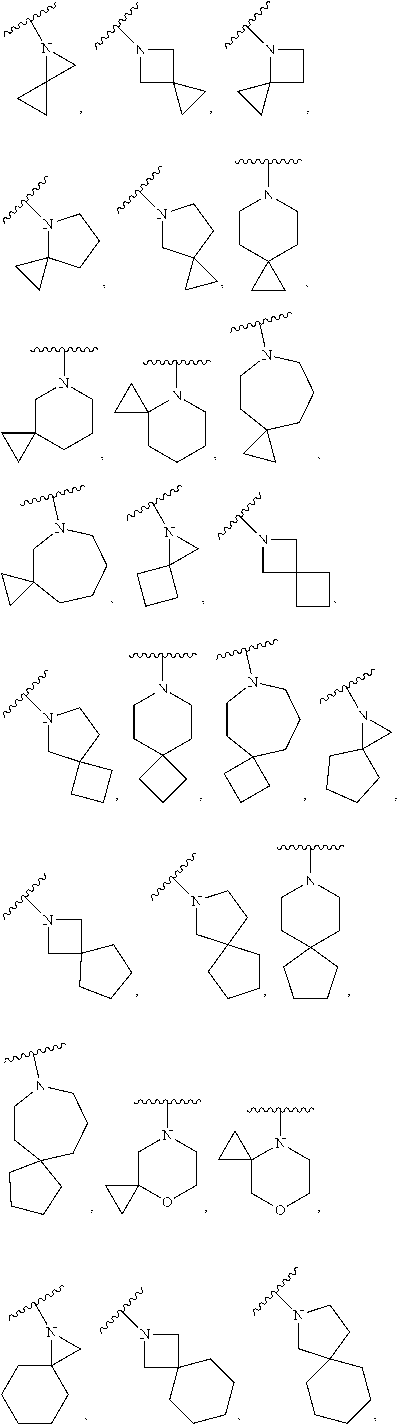

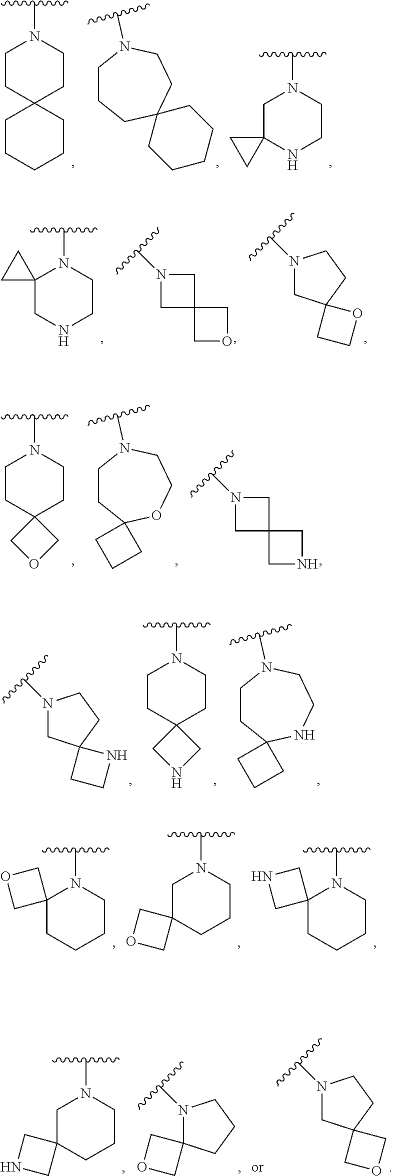

In some embodiments, R³ and R⁴, together with the nitrogen atom to which they are both attached, form a 4-azaspiro[2.4]heptan-4-yl group,

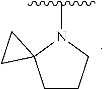 [SMILES: *N1CCCC12CC2]

In some embodiments, R³ and R⁴, together with the nitrogen atom to which they are both attached, form a 5-azaspiro[3.4]octan-5-yl group,

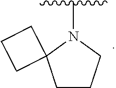 [SMILES: *N1CCCC12CCC2]

In some embodiments, R³ and R⁴, together with the nitrogen atom to which they are both attached, form a 6-azaspiro[3.4]octan-6-yl group,

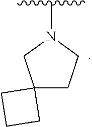 [SMILES: *N1CCC2(CCC2)C1]

In some embodiments, R³ and R⁴, together with the nitrogen atom to which they are both attached, form a 6-azaspiro[2.5]octan-6-yl group,

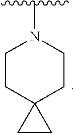 [SMILES: *N1CCC2(CC1)CC2]

In some embodiments, R³ and R⁴, together with the nitrogen atom to which they are both attached, form a 7-azaspiro[3.5]nonan-7-yl group,

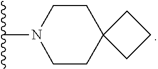 [SMILES: *N1CCC2(CCC2)CC1]

In some embodiments, R³ and R⁴, together with the nitrogen atom to which they are both attached, form a 4-azaspiro[2.5]octan-4-yl group,

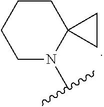 [SMILES: *N1CCCCC12CC2]

In some embodiments, R³ and R⁴, together with the nitrogen atom to which they are both attached, form a 5-azaspiro[2.5]octan-5-yl group,

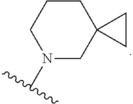 [SMILES: *N1CCCC2(CC2)C1]

In some embodiments, R³ and R⁴, together with the nitrogen atom to which they are both attached, form a 1-azaspiro[3.3]heptan-1-yl group,

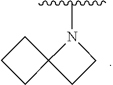 [SMILES: *N1CCC12CCC2]

In some embodiments, R³ and R⁴, together with the nitrogen atom to which they are both attached, form a 2-azaspiro[3.3]heptan-2-yl group,

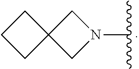 [SMILES: *N1CC2(CCC2)C1]

In some embodiments, R³ and R⁴, together with the nitrogen atom to which they are both attached, form a 7-oxa-2-azaspiro[3.5]nonan-2-yl group,

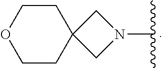 [SMILES: *N1CC2(CCOCC2)C1]

In some embodiments, R³ and R⁴, together with the nitrogen atom to which they are both attached, form a 2-oxa-6-azaspiro[3.3]heptan-6-yl group,

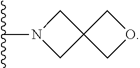 [SMILES: *N1CC2(COC2)C1]

In some embodiments, R³ and R⁴, together with the nitrogen atom to which they are both attached, form a 5-oxa-2-azaspiro[3.5]nonan-2-yl group,

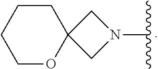 [SMILES: *N1CC2(CCCCO2)C1]

In some embodiments, R³ and R⁴, together with the nitrogen atom to which they are both attached, form a 5-azaspiro[2.4]heptan-5-yl group,

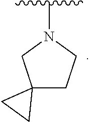 [SMILES: *N1CCC2(CC2)C1]

In some embodiments, R³ and R⁴, together with the nitrogen atom to which they are both attached, form a 2-oxa-5-azaspiro[3.5]nonan-5-yl group,

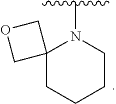 [SMILES: *N1CCCCC12COC2]

In some embodiments, R³ and R⁴, together with the nitrogen atom to which they are both attached, form a 2-oxa-6-azaspiro[3.5]nonan-6-yl group,

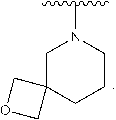 [SMILES: *N1CCCC2(COC2)C1]

In some embodiments, R³ and R⁴, together with the nitrogen atom to which they are both attached, form a 7-azaspiro[4.4]nonan-7-yl group,

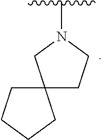 [SMILES: *N1CCC2(CCCC2)C1]

In some embodiments, R³ and R⁴, together with the nitrogen atom to which they are both attached, form a 1-azaspiro[4.4]nonan-1-yl group,

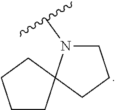 [SMILES: *N1CCCC12CCCC2]

In some embodiments, R³ and R⁴, together with the nitrogen atom to which they are both attached, form a 2-oxa-7-azaspiro[4.4]nonan-7-yl group,

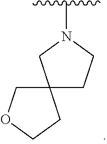 [SMILES: *N1CCC2(CCOC2)C1]

In some embodiments, R³ and R⁴, together with the nitrogen atom to which they are both attached, form a 2-oxa-5-azaspiro[3.4]octan-5-yl group,

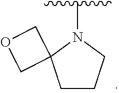 [SMILES: *N1CCCC12COC2]

In some embodiments, R³ and R⁴, together with the nitrogen atom to which they are both attached, form a 2-oxa-6-azaspiro[3.4]octan-6-yl group,

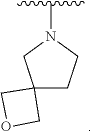 [SMILES: *N1CCC2(COC2)C1]

In some embodiments, R³ and R⁴, together with the nitrogen atom to which they are both attached, form a 2-oxa-7-azaspiro[3.5]nonan-7-yl group,

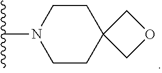 [SMILES: *N1CCC2(CC1)COC2]

In some embodiments, R³ and R⁴, together with the nitrogen atom to which they are both attached, form a 7-oxa-4-azaspiro[2.5]octan-4-yl group,

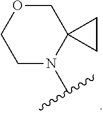 [SMILES: *N1CCOCC12CC2]

In some embodiments, R³ and R⁴, together with the nitrogen atom to which they are both attached, form a 8-oxa-5-azaspiro[3.5]nonan-5-yl group,

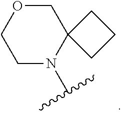 [SMILES: *N1CCOCC12CCC2]

In some embodiments, R³ and R⁴, together with the nitrogen atom to which they are both attached, form a 1-oxa-7-azaspiro[3.5]nonan-7-yl group,

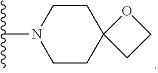 [SMILES: *N1CCC2(CCO2)CC1]

In some aspects, R³ and R⁴ in the compounds of formula (Io) or the compounds of formula (I), together with the nitrogen atom to which they are both attached, form an optionally substituted 4-12-membered fused heterocycloalkyl ring system. Non-limiting examples of such ring systems include:

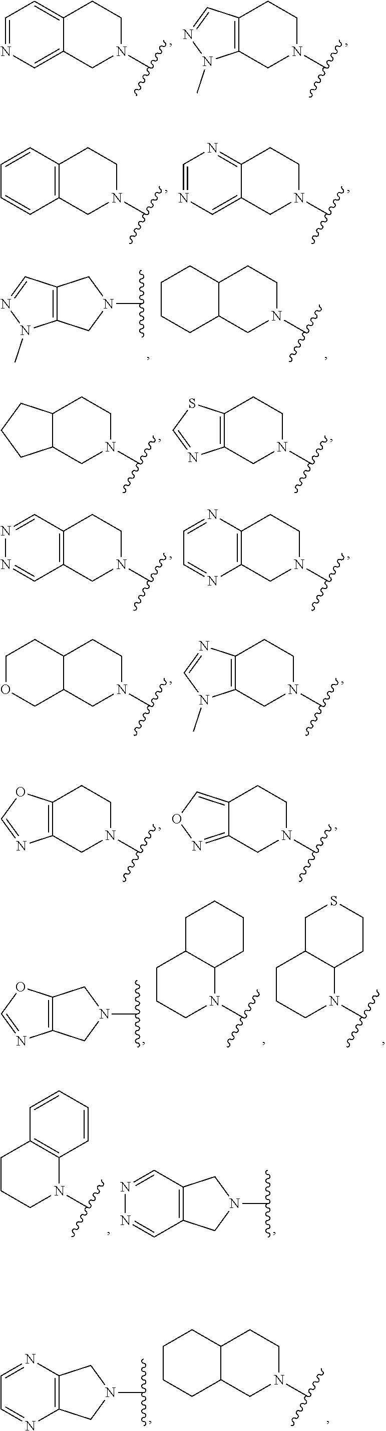

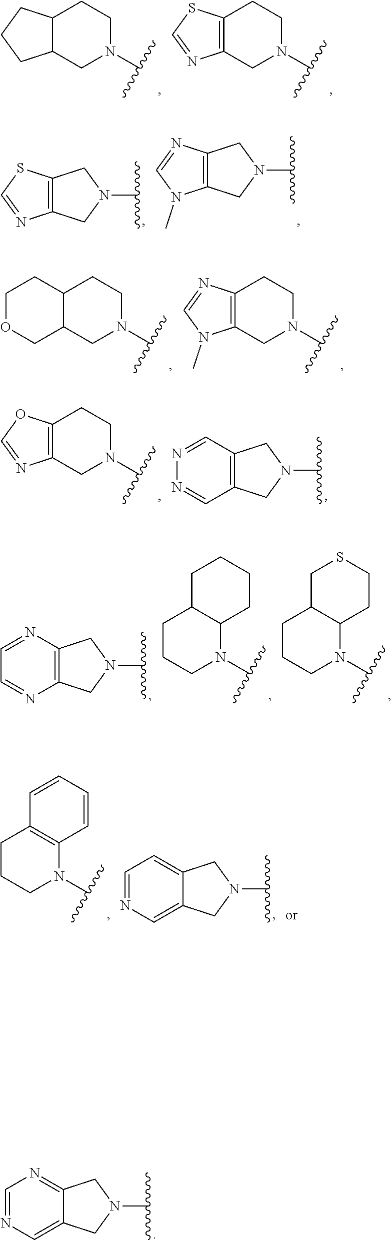

In some embodiments, R³ and R⁴, together with the nitrogen atom to which they are both attached form a 3,4-dihydro-2,7-naphthyridin-2 (1H)-yl group:

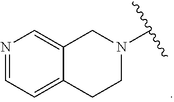 [SMILES: *N1CCc2ccncc2C1]

In some embodiments, R³ and R⁴, together with the nitrogen atom to which they are both attached, form a 1-methyl-4,5-dihydro-1H-pyrazolo[3,4-c]pyridin-6 (7H)-yl group:

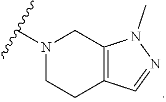 [SMILES: *N1CCc2cnn(C)c2C1]

In some embodiments, R³ and R⁴, together with the nitrogen atom to which they are both attached, form a 5H-pyrrolo[3,4-b]pyridin-6 (7H)-yl group:

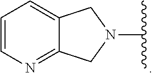 [SMILES: *N1Cc2cccnc2C1]

In some embodiments, R³ and R⁴, together with the nitrogen atom to which they are both attached, form a 1-methyl-6,7-dihydro-1H-pyrazolo[4,3-c]pyridin-5 (4H)-yl group:

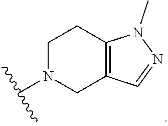 [SMILES: *N1CCc2c(cnn2C)C1]

In some embodiments, R³ and R⁴, together with the nitrogen atom to which they are both attached, form a 6,7-dihydrothiazolo[5,4-c]pyridin-5 (4H)-yl group:

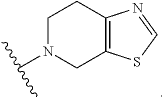 [SMILES: *N1CCc2ncsc2C1]

In some embodiments, R³ and R⁴, together with the nitrogen atom to which they are both attached, form a 5,6-dihydro-1,7-naphthyridin-7 (8H)-yl group:

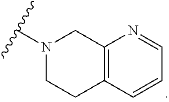 [SMILES: *N1CCc2cccnc2C1]

In some embodiments, R³ and R⁴, together with the nitrogen atom to which they are both attached, form a 6,7-dihydrothiazolo[4,5-c]pyridin-5 (4H)-yl group:

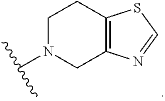 [SMILES: *N1CCc2scnc2C1]

In some embodiments, R³ and R⁴, together with the nitrogen atom to which they are both attached, form a 3,4-dihydro-2,6-naphthyridin-2 (1H)-yl group:

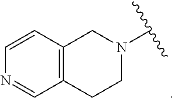 [SMILES: *N1CCc2cnccc2C1]

In some embodiments, R³ and R⁴, together with the nitrogen atom to which they are both attached, form a 7,8-dihydro-1,6-naphthyridin-6 (5H)-yl group:

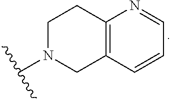 [SMILES: *N1CCc2ncccc2C1]

In some embodiments, R³ and R⁴, together with the nitrogen atom to which they are both attached, form a 5,6-dihydroimidazo[1,2-a]pyrazin-7 (8H)-yl group:

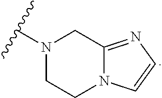 [SMILES: *N1CCn2ccnc2C1]

In some embodiments, R³ and R⁴, together with the nitrogen atom to which they are both attached, form a 4H-pyrrolo[3,4-d]thiazol-5 (6H)-yl group:

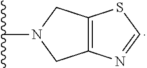 [SMILES: *N1Cc2ncsc2C1]

In some embodiments, R³ and R⁴, together with the nitrogen atom to which they are both attached, form a 1H-pyrrolo[3,4-c]pyridin-2 (3H)-yl group:

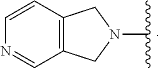 [SMILES: *N1Cc2ccncc2C1]

In some embodiments, R³ and R⁴, together with the nitrogen atom to which they are both attached, form a tetrahydro-1H-furo[3,4-c]pyrrol-5 (3H)-yl group:

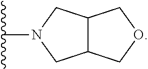 [SMILES: *N1CC2COCC2C1]

In some embodiments, R³ and R⁴, together with the nitrogen atom to which they are both attached, form a 3,4-dihydroisoguinolin-2 (1H)-yl group:

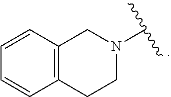 [SMILES: *N1CCc2ccccc2C1]

In some aspects each R⁵ and each R⁶ in the compounds of formula (Io) or the compounds of formula (I) is independently H, C₁-C₆alkyl, or C₃-C₅cycloalkyl; or an R⁵ and R⁶ attached to the same carbon atom, together with that carbon atom, may form a C₃-C₆cycloalkyl ring.

In some embodiments, R⁵ or R⁶ in the compounds of formula (Io) or the compounds of formula (I) is H.

In some embodiments, R⁵ or R⁶ in the compounds of formula (Io) or the compounds of formula (I) is C₁-C₆alkyl, such as, for example, C₁-C₆alkyl, C₁-C₅alkyl, C₁-C₄alkyl, C₁-C₃alkyl, C₁-C₂alkyl, C₆alkyl, C₅alkyl, C₄alkyl, C₃alkyl, C₂alkyl, C₅ alkyl, methyl, ethyl, n-propyl, iso-propyl, n-butyl, t-butyl, iso-butyl, sec-butyl, n-pentyl, n-hexyl, and the like.

In some embodiments, R⁵ or R⁶ is methyl (i.e., —CH₃).

In some embodiments, an R⁵ and an R⁶ are methyl (i.e., —CH₃).

In some embodiments, R⁵ or R⁶ in the compounds of formula (Io) or the compounds of formula (I) is C₃-C₅cycloalkyl, such as, for example, C₃cycloalkyl, C₄cycloalkyl, C₅cycloalkyl, cyclopropyl, cyclobutyl, cyclopentyl, and the like.

In some embodiments, an R⁵ and R⁶ in the compounds of formula (Io) or the compounds of formula (I) attached to the same carbon atom, together with that carbon atom, form a C₃-C₆cycloalkyl ring, such as, for example, C₃cycloalkyl, C₄cycloalkyl, C₅cycloalkyl, C₆cycloalkyl, cyclopropyl, cyclobutyl, cyclopentyl, cyclohexyl, and the like.

In some embodiments, an R⁵ and R⁶ in the compounds of formula (Io) or the compounds of formula (I) attached to the same carbon atom, together with that carbon atom, form a cyclopropyl group.

In some embodiments of the compounds of formula (Io), an R⁵ and R⁶ in attached to the same carbon atom, together with that carbon atom, a C═O.

In some embodiments of the compounds of formula (Io), an R⁵ or R⁶ together with an R³ or R⁴ may form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system.

In some embodiments of the compounds of formula (Io), an R⁵ or R⁶, together with an R³ or R⁴ may form an optionally substituted 3-12-membered heterocycloalkyl ring, such as, for example, an optionally substituted azetidnyl ring, an optionally substituted pyrrolidinyl ring, or an optionally substituted piperidinyl ring.

In some embodiments of the compounds of formula (Io) wherein an R⁵ or R⁶, together with an R³ or R⁴ may form an optionally substituted 3-12-membered heterocycloalkyl ring, the structure

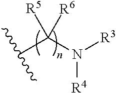

in formula (Io) is

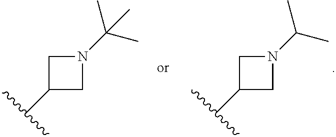

In some embodiments of the compounds of formula (Io) wherein an R⁵ or R⁶, together with an R³ or R⁴ may form an optionally substituted 3-12-membered heterocycloalkyl ring, the structure

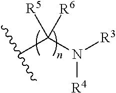

in formula (Io) is

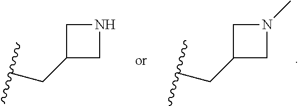

In some embodiments of the compounds of formula (Io) wherein an R⁵ or R⁶, together with an R³ or R⁴ may form an optionally substituted 3-12-membered heterocycloalkyl ring, the structure

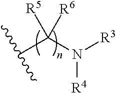

in formula (Io) is

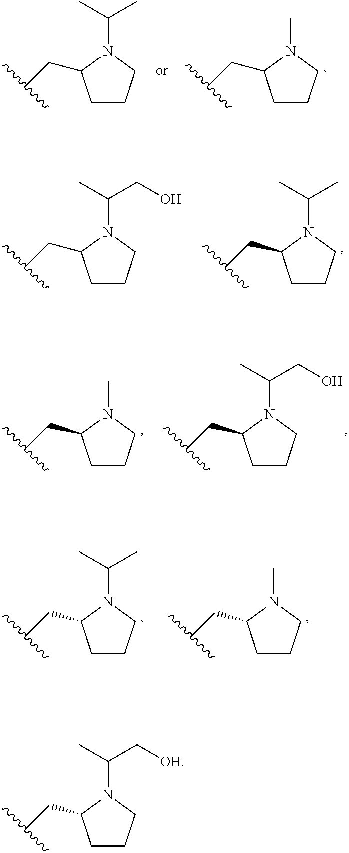

In some embodiments of the compounds of formula (Io) wherein an R⁵ or R⁶, together with an R³ or R⁴ may form an optionally substituted 3-12-membered heterocycloalkyl ring, the structure

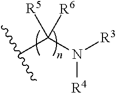

in formula (Io) is

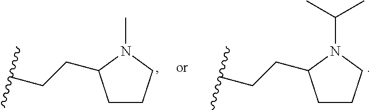

In some embodiments of the compounds of formula (Io) wherein an R⁵ or R⁶, together with an R³ or R⁴ may form an optionally substituted 3-12-membered heterocycloalkyl ring, the structure

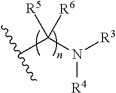

in formula (Io) is

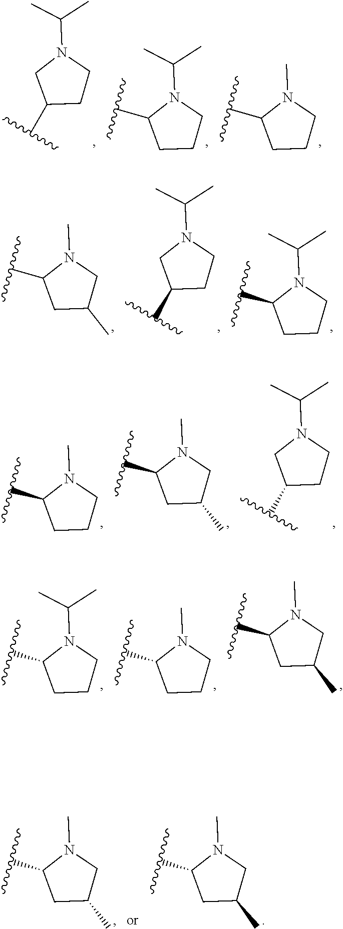

In some embodiments of the compounds of formula (Io) wherein an R⁵ or R⁶, together with an R³ or R⁴ may form an optionally substituted 3-12-membered heterocycloalkyl ring, the structure

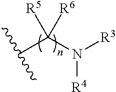

in formula (Io) is

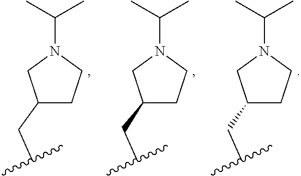

In some embodiments of the compounds of formula (Io) wherein an R⁵ or R⁶, together with an R³ or R⁴ may form an optionally substituted 3-12-membered heterocycloalkyl ring, the structure

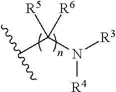

in formula (Io) is

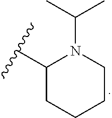 [SMILES: *C1CCCCN1C(C)C]

In some embodiments of the compounds of formula (Io) wherein an R⁵ or R⁶, together with an R³ or R⁴ may form an optionally substituted 3-12-membered heterocycloalkyl ring, the structure

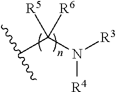

in formula (Io) is

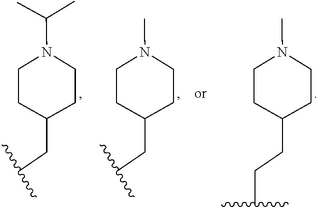

In some embodiments of the compounds of formula (Io) wherein an R⁵ or R⁶, together with an R³ or R⁴ may form an optionally substituted 3-12-membered heterocycloalkyl ring, the structure

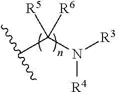

in formula (Io) is

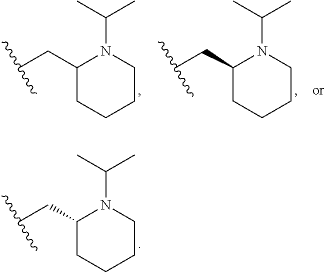

In some embodiments of the compounds of formula (Io), an R⁵ or R⁶ together with an R³ or R⁴ may form an optionally substituted 4-12-membered fused heterocycloalkyl ring system.

In some embodiments of the compounds of formula (Io) wherein an R⁵ or R⁶, together with an R³ or R⁴ may form an optionally substituted 4-12-membered fused heterocycloalkyl ring system, the structure

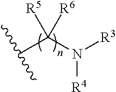

in formula (Io) is

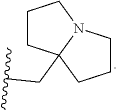 [SMILES: *CC12CCCN1CCC2]

In some embodiments of the compounds of formula (Io) wherein an R⁵ or R⁶, together with an R³ or R⁴ may form an optionally substituted 4-12-membered fused heterocycloalkyl ring system, the structure

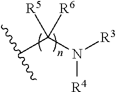

in formula (Io) is

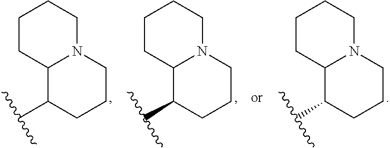

In some embodiments of the compounds of formula (Io), an R⁵ or R⁶, together with an R³ or R⁴ may form an optionally substituted 5-12-membered bridged heterocycloalkyl ring.

In some embodiments of the compounds of formula (Io), an R⁵ or R⁶ together with an R³ or R⁴ may form an optionally substituted 5-12-membered spiroheterocycloalkyl ring system.

In some aspects, the compounds of formula (Io) are compounds of formula (IAo) or formula (IBo):

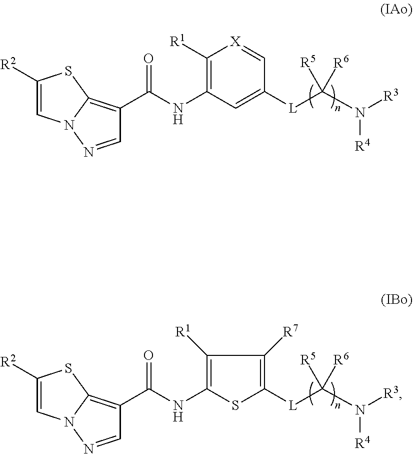

or pharmaceutically acceptable salts thereof, wherein R¹ is H, C₁-C₆alkyl, C₃-C₆cycloalkyl, halogen, —CN, or C₁-C₄fluoroalkyl; R⁷ is H, C₁-C₆alkyl, C₃-C₆cycloalkyl, halogen, —CN, or —CF₃; X is N, or CH; and n, L, R², R³, R⁴, R⁵, and R⁶ are as described above with respect to formula (Io).

In some aspects, the compounds of formula (I) are compounds of formula (IA) or formula (IB):

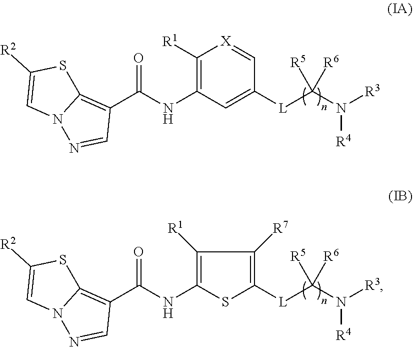

or pharmaceutically acceptable salts thereof, wherein R¹ is H, C₁-C₆alkyl, C₃-C₆cycloalkyl, halogen, —CN, or C₁-C₄fluoroalkyl; R⁷ is H, C₁-C₆alkyl, C₃-C₆cycloalkyl, halogen, —CN, or —CF₃; X is N, or CH; and n, L, R², R³, R⁴, R⁵, and R⁶ are as described above with respect to formula (I).

In some embodiments, the compound is a compound of formula (IAo).

In some embodiments, the compound is a compound of formula (IA).

In some embodiments wherein the compound is a compound of formula (IAo) or a compound of formula (IA), X is N.

In other embodiments wherein the compound is a compound of formula (IAo) or a compound of formula (IA), X is CH.

In some embodiments, the compound is a compound of formula (IBo).

In some embodiments, the compound is a compound of formula (IB).

In some aspects, R¹ in the compounds of formula (IAo), the compounds of formula (IBo), the compounds of formula (IA) or formula (IB) is H, C₁-C₆alkyl, C₃-C₆cycloalkyl, halogen, —CN, or C₁-C₄ fluoroalkyl.

In some embodiments, R¹ in the compounds of formula (IAo), the compounds of formula (IBo), or in the compounds of formula (IA) or formula (IB) is H.

In other embodiments, R¹ in the compounds of formula (IAo), the compounds of formula (IBo), or in the compounds of formula (IA) or formula (IB) is C₁-C₆alkyl, such as, for example, C₁-C₆alkyl, C₁-C₅alkyl, C₁-C₄alkyl, C₁-C₃alkyl, C₁-C₂alkyl, C₆alkyl, C₅alkyl, C₄alkyl, C₃alkyl, C₂alkyl, C₅ alkyl, methyl, ethyl, n-propyl, iso-propyl, n-butyl, t-butyl, iso-butyl, sec-butyl, n-pentyl, n-hexyl, and the like.

In some embodiments, R¹ in the compounds of formula (IAo), the compounds of formula (IBo), or in the compounds of formula (IA) or formula (IB) is C₁-C₆alkyl.

In some embodiments, R¹ in the compounds of formula (IAo), the compounds of formula (IBo), or in the compounds of formula (IA) or formula (IB) is C₁-C₄alkyl.

In some embodiments, R¹ in the compounds of formula (IAo), the compounds of formula (IBo), or in the compounds of formula (IA) or formula (IB) is methyl, i.e., —CH₃.

In some embodiments, R¹ in the compounds of formula (IAo), the compounds of formula (IBo), or in the compounds of formula (IA) or formula (IB) is C₃-C₆cycloalkyl, such as, for example, C₃-C₅cycloalkyl, C₃cycloalkyl, C₄cycloalkyl, C₅cycloalkyl, C₆cycloalkyl, cyclopropyl, cyclobutyl, cyclopentyl, cyclohexyl and the like.

In some embodiments, R¹ in the compounds of formula (IAo), the compounds of formula (IBo), or in the compounds of formula (IA) or formula (IB) is C₃-C₅cycloalkyl.

In some embodiments, R¹ in the compounds of formula (IAo), the compounds of formula (IBo), or in the compounds of formula (IA) or formula (IB) is halogen, such as, —F, —Cl, —Br, or —I.

In some embodiments, R¹ in the compounds of formula (IAo), the compounds of formula (IBo), or in the compounds of formula (IA) or formula (IB) is —CN.

In some embodiments, R¹ in the compounds of formula (IAo), the compounds of formula (IBo), or in the compounds of formula (IA) or formula (IB) is C₁-C₄ fluoroalkyl, such as, for example, C₄ fluoroalkyl, C₃ fluoroalkyl, C₂ fluoroalkyl, C₁ fluoroalkyl, —CF₃, —CHF₂, or —CH₂F.

In some embodiments, R¹ in the compounds of formula (IAo), the compounds of formula (IBo), or in the compounds of formula (IA) or formula (IB) is —CF₃.

In other embodiments, R¹ in the compounds of formula (IAo), the compounds of formula (IBo), or in the compounds of formula (IA) or formula (IB) is —CHF₂.

In some aspects, R⁷ in the compounds of formula (IBo) or the compounds of formula (IB) is H, C₁-C₆alkyl, C₃-C₆cycloalkyl, halogen, —CN, or —CF₃.

In some embodiments, R⁷ in the compounds of formula (IBo) or the compounds of formula (IB) is H.

In other embodiments, R⁷ in the compounds of formula (IBo) or the compounds of formula (IB) is C₁-C₆alkyl, such as, for example, C₁-C₆alkyl, C₁-C₅alkyl, C₁-C₄alkyl, C₁-C₃alkyl, C₁-C₂alkyl, C₆alkyl, C₅alkyl, C₄alkyl, C₃alkyl, C₂alkyl, C₅ alkyl, methyl, ethyl, n-propyl, iso-propyl, n-butyl, t-butyl, iso-butyl, sec-butyl, n-pentyl, n-hexyl, and the like.

In other embodiments, R⁷ in the compounds of formula (IBo) or the compounds of formula (IB) is C₃-C₆cycloalkyl, such as, for example, C₃cycloalkyl, C₄cycloalkyl, C₅cycloalkyl, C₆cycloalkyl, cyclopropyl, cyclobutyl, cyclopentyl, cyclohexyl, and the like.

In some embodiments, R⁷ in the compounds of formula (IBo) or the compounds of formula (IB) is halogen, i.e., —F, —Cl, —Br, or —I.

In some embodiments, R⁷ in the compounds of formula (IBo) or the compounds of formula (IB) is —CN.

In other embodiments, R⁷ in the compounds of formula (IBo) or the compounds of formula (IB) is —CF₃.

In some aspects, the present disclosure provides compounds of formula (IAo) that have the formula (IAo-1):

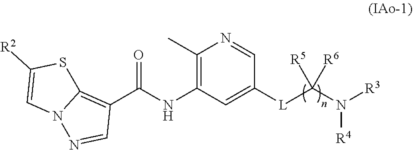

or pharmaceutically acceptable salts thereof, wherein R² is an optionally substituted aryl, optionally substituted heteroaryl, optionally substituted fused heterocycloalkyl, optionally substituted alkyl, optionally substituted alkenyl, or optionally substituted heterocycloalkyl; or one of R³ and R⁴ may be H, or R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; or an R⁵ and R⁶ attached to the same carbon atom, together with that carbon atom, may form a C═O; or an R⁵ or R⁶, together with an R³ or R⁴ may form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system; n is 1, 2, 3, 4, or 5; and L is —NHC(O)— or

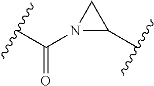 [SMILES: *C(=O)N1CC1*]

when n is 1; or —NHC(O)—, —NHS(O)₂—,

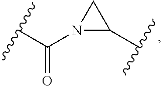 [SMILES: *C(=O)N1CC1*]

—NHC(O)O—, —S(O)₂NH—, —C(O)NH—, or —NHC(O)NH when n is 2,3,4, or 5.

In some embodiments, R² in the compounds of formula (IAo-1) is a pyrrolyl, pyrazolyl, 1,2,3-triazolyl, imidazolyl, isoxazolyl, furanyl, thiophenyl, pyridinyl, pyridazinyl, pyrimidinyl, indazolyl, indolyl, 6,7-dihydro-4H-pyrazolo[5,1-c][1,4]oxazinyl, 5,6-dihydro-8H-imidazo[2,3-c][1,4]oxazinyl, 7,8-dihydro-5H-imidazo[3,2-c][1,3]oxazinyl, 1H-pyrazolo[3,4-b]pyridinyl, 1H-pyrazolo[3,4-b]pyridinyl, 2-oxo-2,3-dihydrobenzo[d]oxazolyl, benzo[d]oxazol-2 (3H)-one-yl, or 5,6-dihydro-4H-pyrrolo[1,2-b]pyrazol-3-yl, each of which may be optionally substituted.

In some embodiments, R² in the compounds of formula (IAo-1) is an optionally substituted pyrazolyl group or an optionally substituted pyridinyl group.

In some embodiments, R² in the compounds of formula (IAo-1) is an optionally substituted phenyl group, an optionally substituted alkyl, an optionally substituted alkenyl, or an optionally substituted heterocycloalkyl.

In some aspects, the present disclosure provides compounds of formula (IA) that have the formula (IA-1):

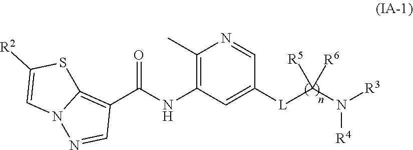

or pharmaceutically acceptable salts thereof, wherein R² is an optionally substituted heteroaryl; R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; n is 1, 2, or 3; and when n is 1, L is —NHC(O)—, and when n is 2 or 3, L is —C(O)NH—, —NHC(O)—, or —NHC(O)NH.

In some embodiments, R² in the compounds of formula (IA-1) is a pyrrolyl, pyrazolyl, 1,2,3-triazolyl, imidazolyl, isoxazolyl, furanyl, thiophenyl, pyridinyl, pyridazinyl, pyrimidinyl, indazolyl, indolyl, 6,7-dihydro-4H-pyrazolo[5,1-c][1,4]oxazinyl, 5,6-dihydro-8H-imidazo[2,3-c][1,4]oxazinyl, 7,8-dihydro-5H-imidazo[3,2-c][1,3]oxazinyl, 1H-pyrazolo[3,4-b]pyridinyl, 1H-pyrazolo[3,4-b]pyridinyl, 2-oxo-2,3-dihydrobenzo[d]oxazolyl, benzo[d]oxazol-2 (3H)-one-yl, or 5,6-dihydro-4H-pyrrolo[1,2-b]pyrazol-3-yl, each of which may be optionally substituted.

In some embodiments, R² in the compounds of formula (IA-1) is an optionally substituted pyrazolyl group or an optionally substituted pyridinyl group.

In some aspects, the present disclosure provides compounds of formula (IAo-1) that have the formula (IAo-2):

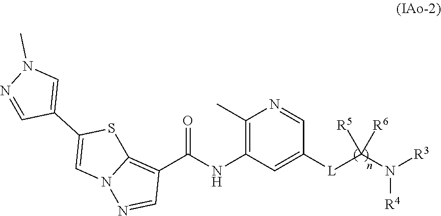

or pharmaceutically acceptable salts thereof, wherein one of R³ and R⁴ may be H, or R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; or an R⁵ and R⁶ attached to the same carbon atom, together with that carbon atom, may form a C═O; or an R⁵ or R⁶, together with an R³ or R⁴ may form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system; n is 1, 2, 3, 4, or 5; and L is —NHC(O)— or

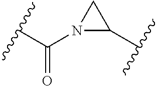 [SMILES: *C(=O)N1CC1*]

when n is 1; or —NHC(O)—, —NHS(O)₂—,

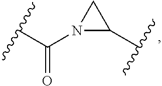 [SMILES: *C(=O)N1CC1*]

—NHC(O)O—, —S(O)₂NH—, —C(O)NH—, or —NHC(O)NH when n is 2,3,4, or 5.

In some embodiments of the compounds of formula (IAo-2), R³ and R⁴, together with the nitrogen atom to which they are both attached, form an optionally substituted 3-12-membered heterocycloalkyl ring.

In some embodiments of the compounds of formula (IAo-2), R³ and R⁴, together with the nitrogen atom to which they are both attached, form an optionally substituted 5-12-membered bridged heterocycloalkyl ring.

In some embodiments of the compounds of formula (IAo-2), R³ and R⁴, together with the nitrogen atom to which they are both attached, form an optionally substituted 4-12-membered fused heterocycloalkyl ring system.

In some embodiments of the compounds of formula (IAo-2), R³ and R⁴, together with the nitrogen atom to which they are both attached, form an optionally substituted 5-12-membered spiroheterocycloalkyl ring system.

In some embodiments of the compounds of formula (IAo-2), an R⁵ or R⁶, together with an R³ or R⁴ form an optionally substituted 3-12-membered heterocycloalkyl ring.

In some embodiments of the compounds of formula (IAo-2), an R⁵ or R⁶, together with an R³ or R⁴ form an optionally substituted 4-12-membered fused heterocycloalkyl ring system.

In some embodiments of the compounds of formula (IAo-2), L is —C(O)NH—, each R⁵ and each R⁶ is H, and n is 2.

In other embodiments of the compounds of formula (IAo-2), L is —NHC(O)—, each R⁵ and each R⁶ is H, and n is 1.

In other embodiments of the compounds of formula (IAo-2), L is —NHC(O)—, each R⁵ and each R⁶ is H, and n is 2.

In other embodiments of the compounds of formula (IAo-2), L is

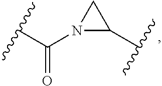 [SMILES: *C(=O)N1CC1*]

and n is 1.

In other embodiments of the compounds of formula (IAo-2), L is —NHC(O)—, and n is 2,3,4, or 5.

In other embodiments of the compounds of formula (IAo-2), L is —NHS(O)₂—, and n is 2,3,4, or 5.

In other embodiments of the compounds of formula (IAo-2), L is

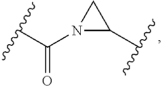 [SMILES: *C(=O)N1CC1*]

and n is 2,3,4, or 5.

In other embodiments of the compounds of formula (IAo-2), L is —NHC(O)O—, and n is 2,3,4, or 5.

In other embodiments of the compounds of formula (IAo-2), L is —S(O)₂NH—, and n is 2,3,4, or 5.

In other embodiments of the compounds of formula (IAo-2), L is —C(O)NH—, and n is 2,3,4, or 5.

In other embodiments of the compounds of formula (IAo-2), L is —NHC(O)NH and n is 2,3,4, or 5.

In some aspects, the present disclosure provides compounds of formula (IA-1) that have the formula (IA-2):

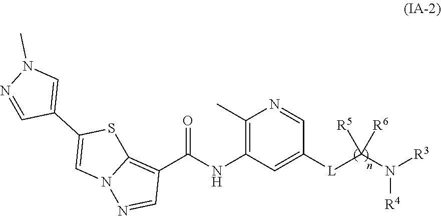

or pharmaceutically acceptable salts thereof, wherein R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; n is 1, 2, or 3; and when n is 1, L is —NHC(O)—, and when n is 2 or 3, L is —C(O)NH—, —NHC(O)—, or —NHC(O)NH.

In some embodiments of the compounds of formula (IA-2), R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N.

In some embodiments of the compounds of formula (IA-2), R³ and R⁴, together with the nitrogen atom to which they are both attached, form an optionally substituted 3-12-membered heterocycloalkyl ring.

In some embodiments of the compounds of formula (IA-2), R³ and R⁴, together with the nitrogen atom to which they are both attached, form an optionally substituted 5-12-membered bridged heterocycloalkyl ring.

In some embodiments of the compounds of formula (IA-2), R³ and R⁴, together with the nitrogen atom to which they are both attached, form an optionally substituted 4-12-membered fused heterocycloalkyl ring system.

In some embodiments of the compounds of formula (IA-2), R³ and R⁴, together with the nitrogen atom to which they are both attached, form an optionally substituted 5-12-membered spiroheterocycloalkyl ring system.

In some embodiments of the compounds of formula (IA-2), L is —C(O)NH—, each R⁵ and each R⁶ is H, and n is 2.

In other embodiments of the compounds of formula (IA-2), L is —NHC(O)—, each R⁵ and each R⁶ is H, and n is 1.

In other embodiments of the compounds of formula (IA-2), L is —NHC(O)—, each R⁵ and each R⁶ is H, and n is 2.

In other aspects, the present disclosure provides compounds of formula (IAo-1) that have the formula (IAo-3):

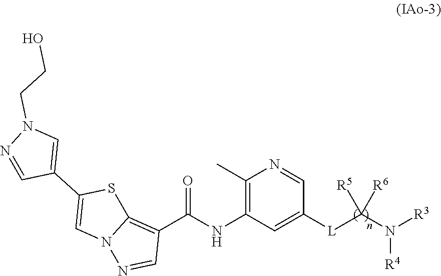

or pharmaceutically acceptable salts thereof, wherein one of R³ and R⁴ may be H, or R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; or an R⁵ and R⁶ attached to the same carbon atom, together with that carbon atom, may form a C═O; or an R⁵ or R⁶, together with an R³ or R⁴ may form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system; n is 1, 2, 3, 4, or 5; and L is —NHC(O)— or

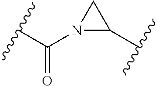 [SMILES: *C(=O)N1CC1*]

when n is 1; or —NHC(O)—, —NHS(O)₂—,

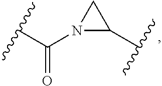 [SMILES: *C(=O)N1CC1*]

—NHC(O)O—, —S(O)₂NH—, —C(O)NH—, or —NHC(O)NH when n is 2,3,4, or 5.

In some embodiments of the compounds of formula (IAo-3), R³ and R⁴, together with the nitrogen atom to which they are both attached, form an optionally substituted 3-12-membered heterocycloalkyl ring.

In some embodiments of the compounds of formula (IAo-3), R³ and R⁴, together with the nitrogen atom to which they are both attached, form an optionally substituted 5-12-membered bridged heterocycloalkyl ring.

In some embodiments of the compounds of formula (IAo-3), R³ and R⁴, together with the nitrogen atom to which they are both attached, form an optionally substituted 4-12-membered fused heterocycloalkyl ring system.

In some embodiments of the compounds of formula (IAo-3), R³ and R⁴, together with the nitrogen atom to which they are both attached, form an optionally substituted 5-12-membered spiroheterocycloalkyl ring system.

In some embodiments of the compounds of formula (IAo-3), an R⁵ or R⁶, together with an R³ or R⁴ form an optionally substituted 3-12-membered heterocycloalkyl ring.

In some embodiments of the compounds of formula (IAo-3), an R⁵ or R⁶, together with an R³ or R⁴ form an optionally substituted 4-12-membered fused heterocycloalkyl ring system.

In some embodiments of the compounds of formula (IAo-3), L is —C(O)NH—, each R⁵ and each R⁶ is H, and n is 2.

In other embodiments of the compounds of formula (IAo-3), L is —NHC(O)—, each R⁵ and each R⁶ is H, and n is 1.

In other embodiments of the compounds of formula (IAo-3), L is —NHC(O)—, each R⁵ and each R⁶ is H, and n is 2.

In other embodiments of the compounds of formula (IAo-3), L is

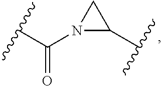 [SMILES: *C(=O)N1CC1*]

and n is 1.

In other embodiments of the compounds of formula (IAo-3), L is —NHC(O)—, and n is 2,3,4, or 5.

In other embodiments of the compounds of formula (IAo-3), L is —NHS(O)₂—, and n is 2,3,4, or 5.

In other embodiments of the compounds of formula (IAo-3), L is

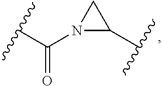 [SMILES: *C(=O)N1CC1*]

and n is 2,3,4, or 5.

In other embodiments of the compounds of formula (IAo-3), L is —NHC(O)O—, and n is 2,3,4, or 5.

In other embodiments of the compounds of formula (IAo-3), L is —S(O)₂NH—, and n is 2,3,4, or 5.

In other embodiments of the compounds of formula (IAo-3), L is —C(O)NH—, and n is 2,3,4, or 5.

In other embodiments of the compounds of formula (IAo-3), L is —NHC(O)NH and n is 2,3,4, or 5.

In other aspects, the present disclosure provides compounds of formula (IA-1) that have the formula (IA-3):

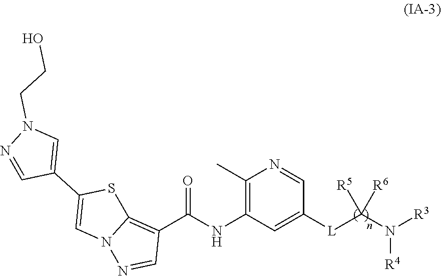

or pharmaceutically acceptable salts thereof, wherein R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; n is 1, 2, or 3; and when n is 1, L is —NHC(O)—, and when n is 2 or 3, L is —C(O)NH—, —NHC(O)—, or —NHC(O)NH.

In some embodiments of the compounds of formula (IA-3), R³ and R⁴, together with the nitrogen atom to which they are both attached, form an optionally substituted 3-12-membered heterocycloalkyl ring.

In some embodiments of the compounds of formula (IA-3), R³ and R⁴, together with the nitrogen atom to which they are both attached, form an optionally substituted 5-12-membered bridged heterocycloalkyl ring.

In some embodiments of the compounds of formula (IA-3), R³ and R⁴, together with the nitrogen atom to which they are both attached, form an optionally substituted 4-12-membered fused heterocycloalkyl ring system.

In some embodiments of the compounds of formula (IA-3), R³ and R⁴, together with the nitrogen atom to which they are both attached, form an optionally substituted 5-12-membered spiroheterocycloalkyl ring system.

In some embodiments of the compounds of formula (IA-3), L is —NHC(O)—, each R⁵ and each R⁶ is H, and n is 1.

In other aspects, the present disclosure provides compounds of formula (IAo-1) that have the formula (IAo-4):

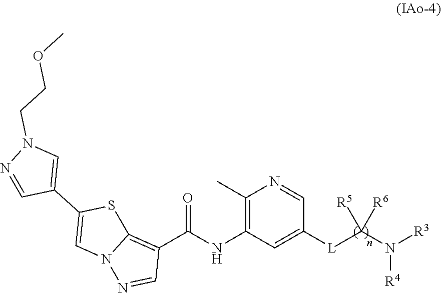

or pharmaceutically acceptable salts thereof, wherein one of R³ and R⁴ may be H, or R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; or an R⁵ and R⁶ attached to the same carbon atom, together with that carbon atom, may form a C═O; or an R⁵ or R⁶, together with an R³ or R⁴ may form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system; n is 1, 2, 3, 4, or 5; and L is —NHC(O)— or

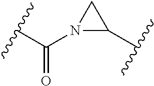 [SMILES: *C(=O)N1CC1*]

when n is 1; or —NHC(O)—, —NHS(O)₂—,

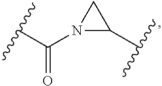 [SMILES: *C(=O)N1CC1*]

—NHC(O)O—, —S(O)₂NH—, —C(O)NH—, or —NHC(O)NH when n is 2,3,4, or 5.

In some embodiments of the compounds of formula (IAo-4), R³ and R⁴, together with the nitrogen atom to which they are both attached, form an optionally substituted 3-12-membered heterocycloalkyl ring.

In some embodiments of the compounds of formula (IAo-4), R³ and R⁴, together with the nitrogen atom to which they are both attached, form an optionally substituted 5-12-membered bridged heterocycloalkyl ring.

In some embodiments of the compounds of formula (IAo-4), R³ and R⁴, together with the nitrogen atom to which they are both attached, form an optionally substituted 4-12-membered fused heterocycloalkyl ring system.

In some embodiments of the compounds of formula (IAo-4), R³ and R⁴, together with the nitrogen atom to which they are both attached, form an optionally substituted 5-12-membered spiroheterocycloalkyl ring system.

In some embodiments of the compounds of formula (IAo-4), an R⁵ or R⁶ together with an R³ or R⁴ form an optionally substituted 3-12-membered heterocycloalkyl ring.

In some embodiments of the compounds of formula (IAo-4), an R⁵ or R⁶ together with an R³ or R⁴ form an optionally substituted 4-12-membered fused heterocycloalkyl ring system.

In some embodiments of the compounds of formula (IAo-4), L is —C(O)NH—, each R⁵ and each R⁶ is H, and n is 2.

In other embodiments of the compounds of formula (IAo-4), L is —NHC(O)—, each R⁵ and each R⁶ is H, and n is 1.

In other embodiments of the compounds of formula (IAo-4), L is —NHC(O)—, each R⁵ and each R⁶ is H, and n is 2.

In other embodiments of the compounds of formula (IAo-4), L is

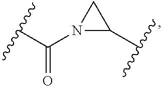 [SMILES: *C(=O)N1CC1*]

and n is 1.

In other embodiments of the compounds of formula (IAo-4), L is —NHC(O)—, and n is 2,3,4, or 5.

In other embodiments of the compounds of formula (IAo-4), L is —NHS(O)₂—, and n is 2,3,4, or 5.

In other embodiments of the compounds of formula (IAo-4), L is

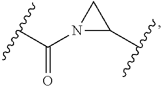 [SMILES: *C(=O)N1CC1*]

and n is 2,3,4, or 5.

In other embodiments of the compounds of formula (IAo-4), L is —NHC(O)O—, and n is 2,3,4, or 5.

In other embodiments of the compounds of formula (IAo-4), L is —S(O)₂NH—, and n is 2,3,4, or 5.

In other embodiments of the compounds of formula (IAo-4), L is —C(O)NH—, and n is 2,3,4, or 5.

In other embodiments of the compounds of formula (IAo-4), L is —NHC(O)NH and n is 2,3,4, or 5.

In other aspects, the present disclosure provides compounds of formula (IA-1) that have the formula (IA-4):

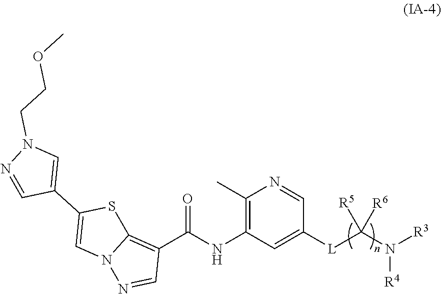

or pharmaceutically acceptable salts thereof, wherein R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; n is 1, 2, or 3; and when n is 1, L is —NHC(O)—, and when n is 2 or 3, L is —C(O)NH—, —NHC(O)—, or —NHC(O)NH.

In some embodiments of the compounds of formula (IA-4), R³ and R⁴, together with the nitrogen atom to which they are both attached, form an optionally substituted 3-12-membered heterocycloalkyl ring.

In some embodiments of the compounds of formula (IA-4), R³ and R⁴, together with the nitrogen atom to which they are both attached, form an optionally substituted 5-12-membered bridged heterocycloalkyl ring.

In some embodiments of the compounds of formula (IA-4), R³ and R⁴, together with the nitrogen atom to which they are both attached, form an optionally substituted 4-12-membered fused heterocycloalkyl ring system.

In some embodiments of the compounds of formula (IA-4), R³ and R⁴, together with the nitrogen atom to which they are both attached, form an optionally substituted 5-12-membered spiroheterocycloalkyl ring system.

In some embodiments of the compounds of formula (IA-4), L is —NHC(O)—, each R⁵ and each R⁶ is H, and n is 1.

In some aspects, the present disclosure provides compounds of formula (IBo) that have the formula (IBo-1):

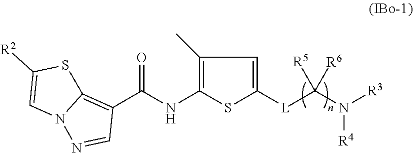

or pharmaceutically acceptable salts thereof, wherein R² is an optionally substituted heteroaryl, optionally substituted fused heterocycloalkyl, optionally substituted alkyl, optionally substituted alkenyl, or optionally substituted heterocycloalkyl; one of R³ and R⁴ may be H, or R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; or an R⁵ and R⁶ attached to the same carbon atom, together with that carbon atom, may form a C═O; or an R⁵ or R⁶, together with an R³ or R⁴ may form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system; n is 1, 2, 3, 4, or 5; and L is —NHC(O)— or

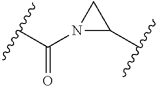 [SMILES: *C(=O)N1CC1*]

when n is 1; or —NHC(O)—, —NHS(O)₂—,

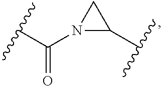 [SMILES: *C(=O)N1CC1*]

—NHC(O)O—, —S(O)₂NH—C(O)NH—, or —NHC(O)NH when n is 2,3,4, or 5.

In some embodiments, R² in the compounds of formula (IBo-1) is a pyrrolyl, pyrazolyl, 1,2,3-triazolyl, imidazolyl, isoxazolyl, furanyl, thiophenyl, pyridinyl, pyridazinyl, pyrimidinyl, indazolyl, indolyl, 6,7-dihydro-4H-pyrazolo[5,1-c][1,4]oxazinyl, 5,6-dihydro-8H-imidazo[2,3-c][1,4]oxazinyl, 7,8-dihydro-5H-imidazo[3,2-c][1,3]oxazinyl, 1H-pyrazolo[3,4-b]pyridinyl, 1H-pyrazolo[3,4-b]pyridinyl, 2-oxo-2,3-dihydrobenzo[d]oxazolyl, benzo[d]oxazol-2 (3H)-one-yl, or 5,6-dihydro-4H-pyrrolo[1,2-b]pyrazol-3-yl, each of which may be optionally substituted.

In some embodiments, R² in the compounds of formula (IBo-1) is an optionally substituted pyrazolyl group or an optionally substituted pyridinyl group.

In some embodiments, L in the compounds of formula (IBo-1) is —C(O)NH— and n is 2 or 3.

In some aspects, the present disclosure provides compounds of formula (IB) that have the formula (IB-1):

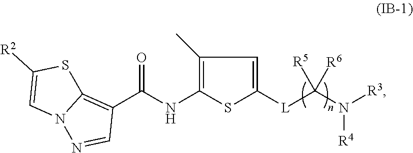

or pharmaceutically acceptable salts thereof, wherein R² is optionally substituted heteroaryl, R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; n is 1, 2, or 3; and when n is 1, L is —NHC(O)—, and when n is 2 or 3, L is —C(O)NH—, —NHC(O)—, or —NHC(O)NH.

In some embodiments, R² in the compounds of formula (IB-1) is a pyrrolyl, pyrazolyl, 1,2,3-triazolyl, imidazolyl, isoxazolyl, furanyl, thiophenyl, pyridinyl, pyridazinyl, pyrimidinyl, indazolyl, indolyl, 6,7-dihydro-4H-pyrazolo[5,1-c][1,4]oxazinyl, 5,6-dihydro-8H-imidazo[2,3-c][1,4]oxazinyl, 7,8-dihydro-5H-imidazo[3,2-c][1,3]oxazinyl, 1H-pyrazolo[3,4-b]pyridinyl, 1H-pyrazolo[3,4-b]pyridinyl, 2-oxo-2,3-dihydrobenzo[d]oxazolyl, benzo[d]oxazol-2 (3H)-one-yl, or 5,6-dihydro-4H-pyrrolo[1,2-b]pyrazol-3-yl, each of which may be optionally substituted.

In some embodiments, R² in the compounds of formula (IB-1) is an optionally substituted pyrazolyl group or an optionally substituted pyridinyl group.

In some embodiments, R² in the compounds of formula (IB-1) is a, pyrazolyl, 1,2,3-triazolyl, or pyridinyl, each of which may be optionally substituted.

In some embodiments, R² in the compounds of formula (IB-1) is a 1-methyl-1H-pyrazol-4-yl.

In some embodiments of the compounds of formula (IB-1), R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N.

In some embodiments of the compounds of formula (IB-1), R³ and R⁴, together with the nitrogen atom to which they are both attached, form an optionally substituted 3-12-membered heterocycloalkyl ring.

In some embodiments of the compounds of formula (IB-1), R³ and R⁴, together with the nitrogen atom to which they are both attached, form an optionally substituted 5-12-membered bridged heterocycloalkyl ring.

In some embodiments of the compounds of formula (IB-1), R³ and R⁴, together with the nitrogen atom to which they are both attached, form an optionally substituted 4-12-membered fused heterocycloalkyl ring system.

In some embodiments of the compounds of formula (IB-1), R³ and R⁴, together with the nitrogen atom to which they are both attached, form an optionally substituted 5-12-membered spiroheterocycloalkyl ring system.

In some embodiments of the compounds of formula (IB-1), L is —C(O)NH—, and n is 2 or 3.

In some embodiments of the compounds of formula (IB-1), L is —C(O)NH—, each R⁵ and each R⁶ is H, and n is 2.

In other embodiments of the compounds of formula (IB-1), L is —NHC(O)—, each R⁵ and each R⁶ is H, and n is 1.

In some aspects, the compounds of formula (I) are compounds of formula (IC):

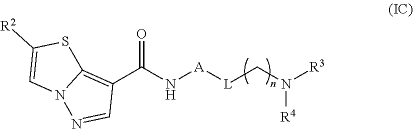

or pharmaceutically acceptable salts thereof, wherein A is a pyridinyl ring substituted with a C₁-C₃alkyl group or thiophenyl ring substituted with a C₁-C₃alkyl group; R² is a 5-6 membered heteroaryl ring containing 1-2 nitrogen (N) atoms and optionally substituted with a C₁-C₃alkyl group, a hydroxy-substituted C₂alkyl group, or a C₃-C₅cycloalkyl group; R³ and R⁴ are each independently C₁-C₃alkyl, or 6-membered heterocycloalkyl containing 1 oxygen (O) atom; or R³ and R⁴, together with the nitrogen atom to which they are both attached, form a 4-6-membered heterocycloalkyl ring optionally substituted with 1-2 C₁-C₃alkyl groups or 1-2 fluorine (F) atoms; a 7-9-membered bridged heterocycloalkyl ring, a 9-10-membered fused heterocycloalkyl ring system optionally substituted with a C₁-C₃alkyl group, or a 7-9-membered spiroheterocycloalkyl ring system, wherein the 9-10-membered fused heterocycloalkyl ring system, or the 7-9-membered spiroheterocycloalkyl ring system, may include, in addition to the nitrogen atom to which both R³ and R⁴ are attached, 1-2 other heteroatoms that are each independently O or N; n is 1 or 2; and when n is 1, L is —NHC(O)—, and when n is 2, L is —C(O)NH—, —NHC(O)—, or —NHC(O)NH.

In some embodiments, the compounds of formula (IC) are compounds of formula (IC-1):

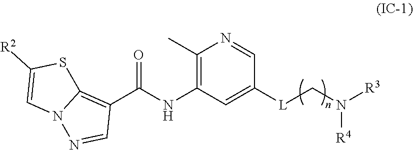

or pharmaceutically acceptable salts thereof, wherein R² is a pyridinyl ring or a pyrazolyl ring, wherein the pyrazolyl ring is optionally substituted with a C₁-C₃alkyl group, a C₃-C₅cycloalkyl group, or a hydroxy-substituted C₂ alkyl group; R³ and R⁴ are each independently C₁-C₃alkyl, or 6-membered heterocycloalkyl containing 1 oxygen (O) atom; or R³ and R⁴, together with the nitrogen atom to which they are both attached, form a 4-6-membered heterocycloalkyl ring optionally substituted with 1-2 C₁-C₃alkyl groups or 1-2 —F atoms; a 7-9-membered bridged heterocycloalkyl ring, a 9-10-membered fused heterocycloalkyl ring system optionally substituted with a C₁-C₃alkyl group, or a 7-9-membered spiroheterocycloalkyl ring system, wherein the 9-10-membered fused heterocycloalkyl ring system or the 7-9-membered spiroheterocycloalkyl ring system may include, in addition to the nitrogen atom to which both R³ and R⁴ are attached, 1-2 other heteroatoms that are each independently O or N; n is 1 or 2; and when n is 1, L is —NHC(O)—, and when n is 2, L is —C(O)NH—, —NHC(O)—, or —NHC(O)NH.

In some embodiments, the compounds of formula (IC) are compounds of formula (IC-2):

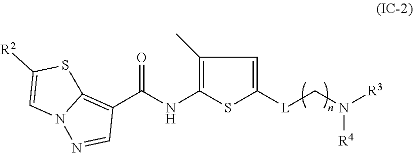

or pharmaceutically acceptable salts thereof, wherein R² is a pyridinyl ring or a pyrazolyl ring, wherein the pyrazolyl ring is optionally substituted with a C₁-C₃alkyl group, R³ and R⁴, together with the nitrogen atom to which they are both attached, form a 5-membered heterocycloalkyl ring substituted with 2 C₁-C₃alkyl groups, or a 7-8-membered spiroheterocycloalkyl ring system, n is 2; and L is —C(O)NH—.

In some aspects, the compounds of formula (Io) are compounds of formula (IDo):

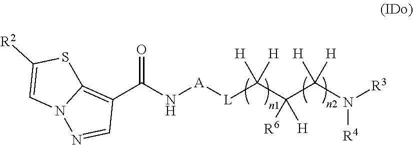

wherein n1 is 0, 1, or 2; n2 is 0, 1 or 2; R³ is H or optionally substituted alkyl; R⁴ and R⁶, together with the atoms to which they are attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system; and R², A, and L are as set forth above with respect to compounds of formula (Io).

In some embodiments of the compound of formula (IDo), n1 is 0.

In some embodiments of the compound of formula (IDo), n1 is 1.

In some embodiments of the compound of formula (IDo), n1 is 2.

In some embodiments of the compound of formula (IDo), n2 is 0.

In some embodiments of the compound of formula (IDo), n2 is 1.

In some embodiments of the compound of formula (IDo), n2 is 2.

In some embodiments of the compound of formula (IDo), n1 is 0 and n2 is 0. (2:1)

In some embodiments of the compound of formula (IDo), n1 is 0 and n2 is 1. (2:1)

In some embodiments of the compound of formula (IDo), n1 is 1 and n2 is 0. (2:2)

In some embodiments of the compound of formula (IDo), n1 is 1 and n2 is 1. (3:2)

In some embodiments of the compound of formula (IDo), n1 is 1 and n2 is 2. (4:2)

In some embodiments of the compound of formula (IDo), n1 is 2 and n2 is 0. (3:3)

In some embodiments of the compound of formula (IDo), n1 is 2 and n2 is 2. (5:3).

In some embodiments of the compound of formula (IDo), R⁴ and R⁶, together with the atoms to which they are attached, form an optionally substituted 3-12-membered heterocycloalkyl ring.

In some embodiments of the compound of formula (IDo), R⁴ and R⁶, together with the atoms to which they are attached, form a 6-membered heterocycloalkyl ring.

In some embodiments of the compound of formula (IDo), R⁴ and R⁶, together with the atoms to which they are attached, form a 5-membered heterocycloalkyl ring.

In some embodiments of the compound of formula (IDo), R⁴ and R⁶, together with the atoms to which they are attached, form a 4-membered heterocycloalkyl ring.

In some embodiments of the compound of formula (IDo), R⁴ and R⁶, together with the atoms to which they are attached, form an optionally substituted 4-12-membered fused heterocycloalkyl ring system.

In some embodiments of the compound of formula (IDo), R⁴ and R⁶, together with the atoms to which they are attached, form an optionally substituted 8-10-membered fused heterocycloalkyl ring system.

In some embodiments of the compound of formula (IDo), R³ is —CH₃ or —CH(CH₃)₂.

In some embodiments of the compound of formula (IDo), R³ is —CH₃ or —CH(CH₃)₂.

In some embodiments of the compound of formula (IDo), L is —NHC(O)—.

In some embodiments of the compound of formula (IDo), L is —NHC(O)—, —NHC(O)O—, —C(O)NH—, or —NHC(O)NH—.

In other embodiments, A in formula (IDo) or formula (I) is an optionally substituted pyridinyl ring.

In some embodiments, A in formula (IDo) or formula (I) is an optionally substituted thiophene.

In some aspects, the compounds of formula (Io) are compounds of formula (IEo)

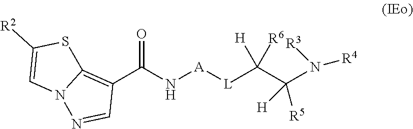

wherein R⁴ and R⁶, together with the atoms to which they are attached, and R³ and R⁵, together with the atoms to which they are attached, together form an optionally substituted 4-12-membered fused heterocycloalkyl ring system; and R², A, and L are as set forth above with respect to compounds of formula (Io).

In other embodiments, A in formula (IEo) is an optionally substituted pyridinyl ring.

In some embodiments, A in formula (IEo) is an optionally substituted thiophene.

In some aspects, the compounds of formula (Io) are compounds of formula (IFo)

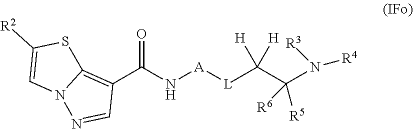

wherein R⁴ and R⁶, together with the atoms to which they are attached, and R³ and R⁵, together with the atoms to which they are attached, together form an optionally substituted 4-12-membered fused heterocycloalkyl ring system; and R², A, and L are as set forth above with respect to compounds of formula (Io).

In other embodiments, A in formula (IFo) is an optionally substituted pyridinyl ring.

In some embodiments, A in formula (IFo) is an optionally substituted thiophene.

In some aspects, the disclosure is directed to the compounds listed in Table 1, or pharmaceutically acceptable salts thereof:

TABLE 1

| Ex. | | |
|---|---|---|
| # | Structure | Chemical Name |
| 1 | 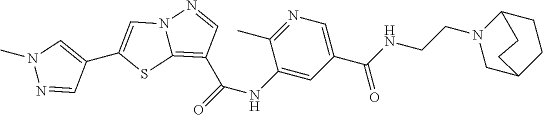 [SMILES: Cc1ncc(C(=O)NCCN2CC3CCC2CC3)cc1NC(=O)c1cnn2cc(-c3cnn(C)c3)sc12] | N-(5-((2-(2- azabicyclo[2.2.2]octan-2- yl)ethyl)carb-amoyl)-2- methylpyridin-3-yl)-2-(1- methyl-1H-pyrazol-4- yl)pyrazolo[5,1-b]thiazole-7- carboxamide |
| 2 | 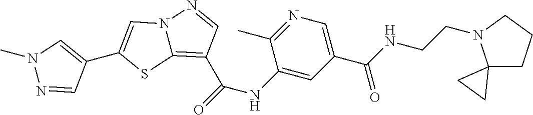 [SMILES: Cc1ncc(C(=O)NCCN2CCCC23CC3)cc1NC(=O)c1cnn2cc(-c3cnn(C)c3)sc12] | N-(5-((2-(4-azaspiro[2.4]heptan- 4-yl)ethyl)carbamoyl)-2- methylpyridin-3-yl)-2-(1- methyl-1H-pyrazol-4- yl)pyrazolo[5,1-b]thiazole-7- carboxamide |
| 3 | 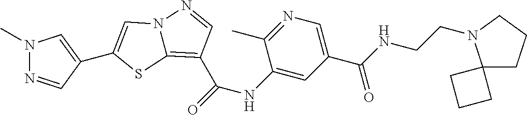 [SMILES: Cc1ncc(C(=O)NCCN2CCCC23CCC3)cc1NC(=O)c1cnn2cc(-c3cnn(C)c3)sc12] | N-(5-((2-(5-azaspiro[3.4]octan- 5-yl)ethyl)carbamoyl)-2- methylpyridin-3-yl)-2-(1- methyl-1H-pyrazol-4- yl)pyrazolo[5,1-b]thiazole-7- carboxamide |
| 4 | 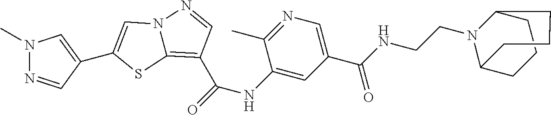 [SMILES: Cc1ncc(C(=O)NCCN2C3CCCC2CCC3)cc1NC(=O)c1cnn2cc(-c3cnn(C)c3)sc12] | N-(5-((2-(9- azabicyclo[3.3.1]nonan-9- yl)ethyl)carbamoyl)-2- methylpyridin-3-yl)-2-(1- methyl-1H-pyrazol-4- yl)pyrazolo[5,1-b]thiazole-7- carboxamide |
| 5 | 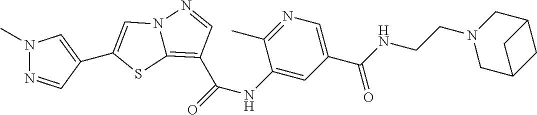 [SMILES: Cc1ncc(C(=O)NCCN2CC3CC(C3)C2)cc1NC(=O)c1cnn2cc(-c3cnn(C)c3)sc12] | N-(5-((2-(3- azabicyclo[3.1.1]heptan-3- yl)ethyl)carbamoyl)-2- methylpyridin-3-yl)-2-(1- methyl-1H-pyrazol-4- yl)pyrazolo[5,1-b]thiazole-7- carboxamide |
| 6 | 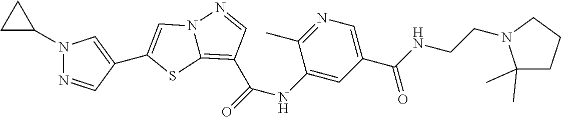 [SMILES: Cc1ncc(C(=O)NCCN2CCCC2(C)C)cc1NC(=O)c1cnn2cc(-c3cnn(C4CC4)c3)sc12] | 2-(1-cyclopropyl-1H-pyrazol-4- yl)-N-(5-((2-(2,2- dimethylpyrroli-din-1- yl)ethyl)carbamoyl)-2- methylpyridin-3- yl)pyrazolo[5,1-b]thiazole-7- carboxamide |
| 7 | 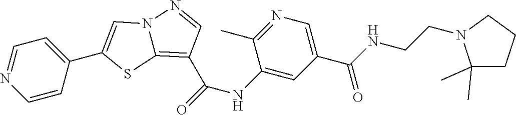 [SMILES: Cc1ncc(C(=O)NCCN2CCCC2(C)C)cc1NC(=O)c1cnn2cc(-c3ccncc3)sc12] | N-(5-((2-(2,2- dimethylpyrrolidin-1- yl)ethyl)carbamoyl)-2-methyl- pyridin-3-yl)-2-(pyridin-4- yl)pyrazolo[5,1-b]thiazole-7- carboxamide |
| 8 | 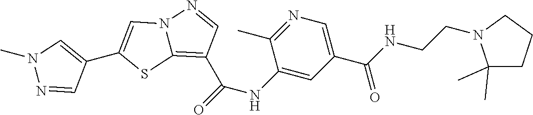 [SMILES: Cc1ncc(C(=O)NCCN2CCCC2(C)C)cc1NC(=O)c1cnn2cc(-c3cnn(C)c3)sc12] | N-(5-((2-(2,2- dimethylpyrrolidin-1- yl)ethyl)carbamoyl)-2- methylpyridin-3-yl)-2-(1- methyl-1H-pyrazol-4- yl)pyrazolo[5,1-b]thiazole-7- carboxamide |
| 9 | 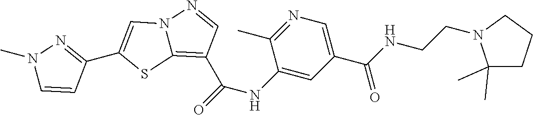 [SMILES: Cc1ncc(C(=O)NCCN2CCCC2(C)C)cc1NC(=O)c1cnn2cc(-c3ccn(C)n3)sc12] | N-(5-((2-(2,2- dimethylpyrrolidin-1- yl)ethyl)carbamoyl)-2- methylpyridin-3-yl)-2-(1- methyl-1H-pyrazol-3- yl)pyrazolo[5,1-b]thiazole-7- carboxamide |
| 10 | 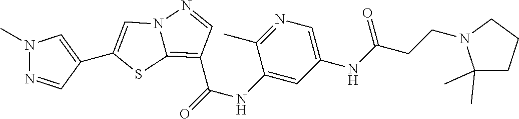 [SMILES: Cc1ncc(NC(=O)CCN2CCCC2(C)C)cc1NC(=O)c1cnn2cc(-c3cnn(C)c3)sc12] | N-(5-(3-(2,2-dimethylpyrrolidin- 1-yl)propanamido)-2-methyl- pyridin-3-yl)-2-(1-methyl-1H- pyrazol-4-yl)pyrazolo[5,1- b]thiazole-7-carboxamide |
| 11 | 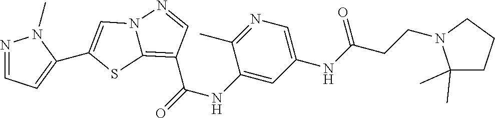 [SMILES: Cc1ncc(NC(=O)CCN2CCCC2(C)C)cc1NC(=O)c1cnn2cc(-c3ccnn3C)sc12] | N-(5-(3-(2,2-dimethylpyrrolidin- 1-yl)propanamido)-2-methyl- pyridin-3-yl)-2-(1-methyl-1H- pyrazol-5-yl)pyrazolo[5,1- b]thiazole-7-carboxamide |
| 12 | 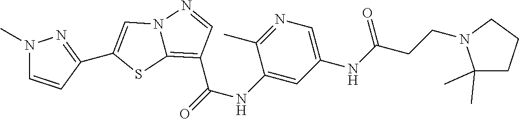 [SMILES: Cc1ncc(NC(=O)CCN2CCCC2(C)C)cc1NC(=O)c1cnn2cc(-c3ccn(C)n3)sc12] | N-(5-(3-(2,2-dimethylpyrrolidin- 1-yl)propanamido)-2-methyl- pyridin-3-yl)-2-(1-methyl-1H- pyrazol-3-yl)pyrazolo[5,1- b]thiazole-7-carboxamide |
| 13 | 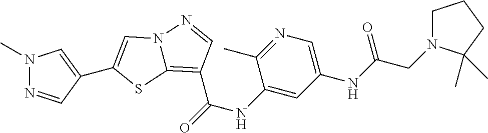 | N-(5-(2-(2,2-dimethylpyrrolidin- 1-yl)acetamido)-2- methylpyridin-3-yl)-2-(1- methyl-1H-pyrazol-4- yl)pyrazolo[5,1-b]thiazole-7- carboxamide |
| 14 | 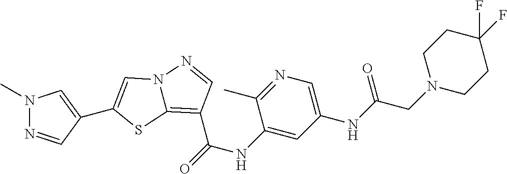 [SMILES: Cc1ncc(NC(=O)CN2CCC(F)(F)CC2)cc1NC(=O)c1cnn2cc(-c3cnn(C)c3)sc12] | N-(5-(2-(4,4-difluoropiperidin-1- yl)acetamido)-2-methylpyridin- 3-yl)-2-(1-methyl-1H-pyrazol-4- yl)pyrazolo[5,1-b]thiazole-7- carboxamide |
| 15 | 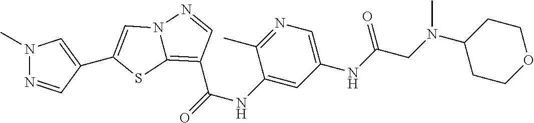 [SMILES: Cc1ncc(NC(=O)CN(C)C2CCOCC2)cc1NC(=O)c1cnn2cc(-c3cnn(C)c3)sc12] | 2-(1-methyl-1H-pyrazol-4-yl)- N-(2-methyl-5-(2- (methyl(tetrahydro-2H-pyran-4- yl)amino)acetamido)pyridin-3- yl)pyrazolo[5,1-b]thiazole-7- carboxamide |
| 16 | 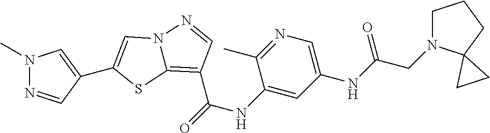 [SMILES: Cc1ncc(NC(=O)CN2CCCC23CC3)cc1NC(=O)c1cnn2cc(-c3cnn(C)c3)sc12] | N-(5-(2-(4-azaspiro[2.4]heptan- 4-yl)acetamido)-2- methylpyridin-3-yl)-2-(1- methyl-1H-pyrazol-4- yl)pyrazolo[5,1-b]thiazole-7- carboxamide |
| 17 | 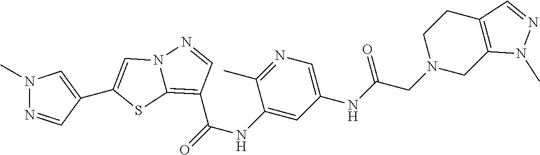 | 2-(1-methyl-1H-pyrazol-4-yl)- N-(2-methyl-5-(2-(1-methyl-4,5- dihydro-1H-pyrazolo[3,4- c]pyridin-6(7H)- yl)acetamido)pyridin-3- yl)pyrazolo[5,1-b]thiazole-7- carboxamide |
| 18 | 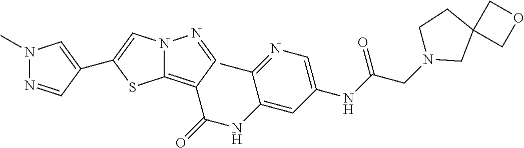 [SMILES: Cc1ncc(NC(=O)CN2CCC3(COC3)C2)cc1NC(=O)c1cnn2cc(-c3cnn(C)c3)sc12] | N-(5-(2-(2-oxa-6- azaspiro[3.4]octan-6- yl)acetamido)-2-methylpyridin- 3-yl)-2-(1-methyl-1H-pyrazol-4- yl)pyrazolo[5,1-b]thiazole-7- carboxamide |
| 19 | 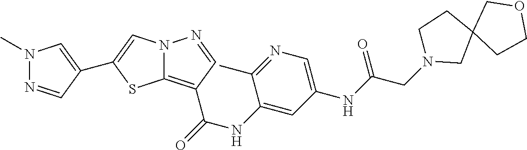 [SMILES: Cc1ncc(NC(=O)CN2CCC3(CCOC3)C2)cc1NC(=O)c1cnn2cc(-c3cnn(C)c3)sc12] | N-(5-(2-(2-oxa-7- azaspiro[4.4]nonan-7- yl)acetamido)-2-methylpyridin- 3-yl)-2-(1-methyl-1H-pyrazol-4- yl)pyrazolo[5,1-b]thiazole-7- carboxamide |
| 20 | 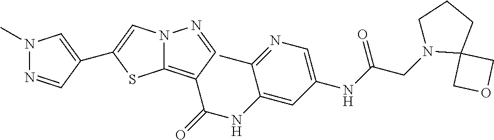 [SMILES: Cc1ncc(NC(=O)CN2CCCC23COC3)cc1NC(=O)c1cnn2cc(-c3cnn(C)c3)sc12] | N-(5-(2-(2-oxa-5- azaspiro[3.4]octan-5- yl)acetamido)-2-methyl-pyridin- 3-yl)-2-(1-methyl-1H-pyrazol-4- yl)pyrazolo[5,1-b]thiazole-7- carboxamide |
| 21 | 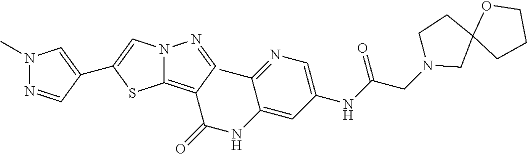 [SMILES: Cc1ncc(NC(=O)CN2CCC3(CCCO3)C2)cc1NC(=O)c1cnn2cc(-c3cnn(C)c3)sc12] | N-(5-(2-(1-oxa-7- azaspiro[4.4]nonan-7- yl)acetamido)-2-methylpyridin- 3-yl)-2-(1-methyl-1H-pyrazol-4- yl)pyrazolo[5,1-b]thiazole-7- carboxamide |
| 22 | 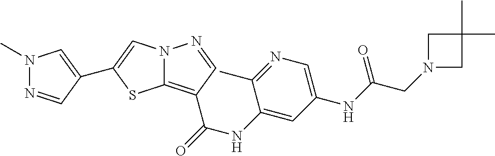 | N-(5-(2-(3,3-dimethylazetidin-1- yl)acetamido)-2-methylpyridin- 3-yl)-2-(1-methyl-1H-pyrazol-4- yl)pyrazolo[5,1-b]thiazole-7- carboxamide |
| 23 | 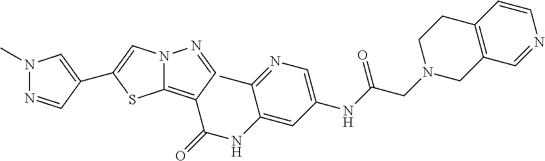 [SMILES: Cc1ncc(NC(=O)CN2CCc3ccncc3C2)cc1NC(=O)c1cnn2cc(-c3cnn(C)c3)sc12] | N-(5-(2-(3,4-dihydro-2,7- naphthyridin-2(1H)- yl)acetamido)-2-methylpyridin- 3-yl)-2-(1-methyl-1H-pyrazol-4- yl)pyrazolo[5,1-b]thiazole-7- carboxamide |
| 24 | 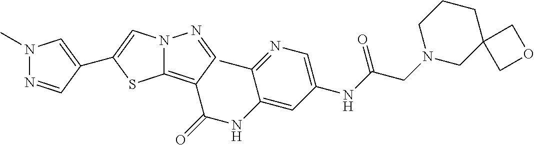 [SMILES: Cc1ncc(NC(=O)CN2CCCC3(COC3)C2)cc1NC(=O)c1cnn2cc(-c3cnn(C)c3)sc12] | N-(5-(2-(2-oxa-6- azaspiro[3.5]nonan-6- yl)acetamido)-2-methylpyridin- 3-yl)-2-(1-methyl-1H-pyrazol-4- yl)pyrazolo[5,1-b]thiazole-7- carboxamide |
| 25 | 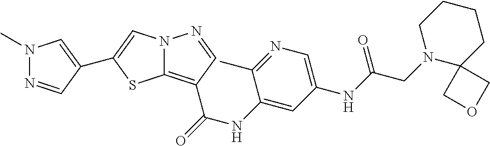 [SMILES: Cc1ncc(NC(=O)CN2CCCCC23COC3)cc1NC(=O)c1cnn2cc(-c3cnn(C)c3)sc12] | N-(5-(2-(2-oxa-5- azaspiro[3.5]nonan-5- yl)acetamido)-2-methylpyridin- 3-yl)-2-(1-methyl-1H-pyrazol-4- yl)pyrazolo[5,1-b]thiazole-7- carboxamide |
| 26 | 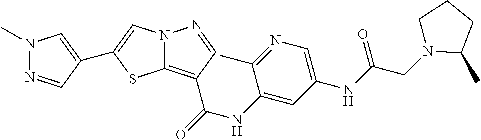 | (R)-2-(1-methyl-1H-pyrazol-4- yl)-N-(2-methyl-5-(2-(2-methyl- pyrrolidin-1- yl)acetamido)pyridin-3- yl)pyrazolo[5,1-b]thiazole-7- carboxamide |
| 27 | 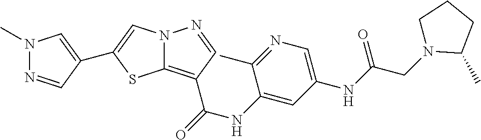 | (S)-2-(1-methyl-1H-pyrazol-4- yl)-N-(2-methyl-5-(2-(2-methyl- pyrrolidin-1- yl)acetamido)pyridin-3- yl)pyrazolo[5,1-b]thiazole-7- carboxamide |
| 28 | 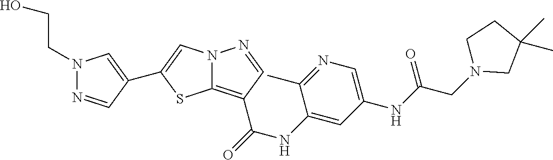 [SMILES: Cc1ncc(NC(=O)CN2CCC(C)(C)C2)cc1NC(=O)c1cnn2cc(-c3cnn(CCO)c3)sc12] | N-(5-(2-(3,3-dimethylpyrrolidin- 1-yl)acetamido)-2- methylpyridin-3-yl)-2-(1-(2- hydroxyethyl)-1H-pyrazol-4- yl)pyrazolo[5,1-b]thiazole-7- carboxamide |
| 29 | 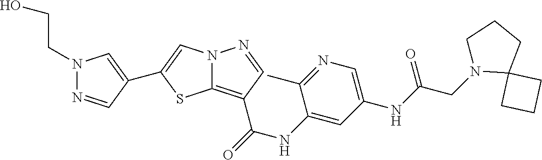 [SMILES: Cc1ncc(NC(=O)CN2CCCC23CCC3)cc1NC(=O)c1cnn2cc(-c3cnn(CCO)c3)sc12] | N-(5-(2-(5-azaspiro[3.4]octan-5- yl)acetamido)-2-methylpyridin- 3-yl)-2-(1-(2-hydroxyethyl)-1H- pyrazol-4-yl)pyrazolo[5,1- b]thiazole-7-carboxamide |
| 30 | 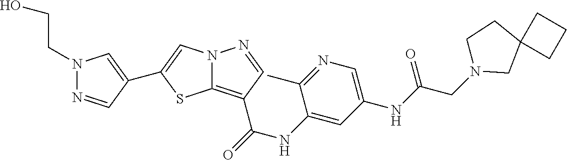 [SMILES: Cc1ncc(NC(=O)CN2CCC3(CCC3)C2)cc1NC(=O)c1cnn2cc(-c3cnn(CCO)c3)sc12] | N-(5-(2-(6-azaspiro[3.4]octan-6- yl)acetamido)-2-methylpyridin- 3-yl)-2-(1-(2-hydroxyethyl)-1H- pyrazol-4-yl)pyrazolo[5,1- b]thiazole-7-carboxamide |
| 31 | 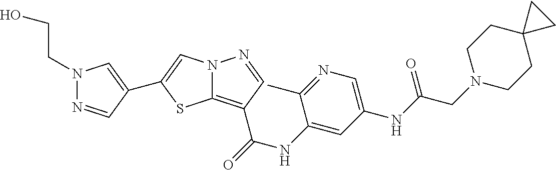 [SMILES: Cc1ncc(NC(=O)CN2CCC3(CC2)CC3)cc1NC(=O)c1cnn2cc(-c3cnn(CCO)c3)sc12] | N-(5-(2-(6-azaspiro[2.5]octan-6- yl)acetamido)-2-methylpyridin- 3-yl)-2-(1-(2-hydroxyethyl)-1H- pyrazol-4-yl)pyrazolo[5,1- b]thiazole-7-carboxamide |
| 32 | 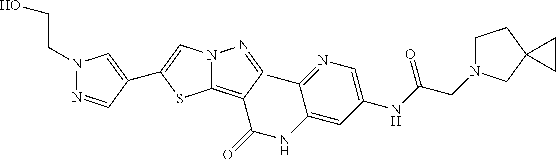 [SMILES: Cc1ncc(NC(=O)CN2CCC3(CC3)C2)cc1NC(=O)c1cnn2cc(-c3cnn(CCO)c3)sc12] | N-(5-(2-(5-azaspiro[2.4]heptan- 5-yl)acetamido)-2- methylpyridin-3-yl)-2-(1-(2- hydroxyethyl)-1H-pyrazol-4- yl)pyrazolo[5,1-b]thiazole-7- carboxamide |
| 33 | 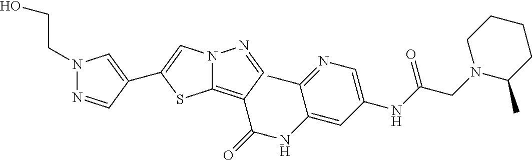 [SMILES: Cc1ncc(NC(=O)CN2CCCC[C@H]2C)cc1NC(=O)c1cnn2cc(-c3cnn(CCO)c3)sc12] | (R)-2-(1-(2-hydroxyethyl)-1H- pyrazol-4-yl)-N-(2-methyl-5-(2- (2-methyl-piperidin-1- yl)acetamido)pyridin-3- yl)pyrazolo[5,1-b]thiazole-7- carboxamide |
| 34 | 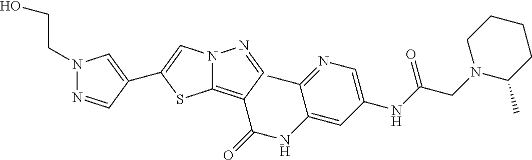 [SMILES: Cc1ncc(NC(=O)CN2CCCC[C@@H]2C)cc1NC(=O)c1cnn2cc(-c3cnn(CCO)c3)sc12] | (S)-2-(1-(2-hydroxyethyl)-1H- pyrazol-4-yl)-N-(2-methyl-5-(2- (2-methyl-piperidin-1- yl)acetamido)pyridin-3- yl)pyrazolo[5,1-b]thiazole-7- carboxamide |
| 35 | 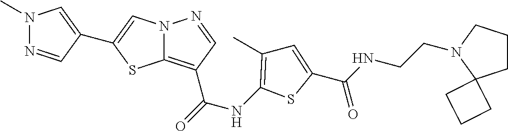 [SMILES: Cc1cc(C(=O)NCCN2CCCC23CCC3)sc1NC(=O)c1cnn2cc(-c3cnn(C)c3)sc12] | N-(5-((2-(5-azaspiro[3.4]octan- 5-yl)ethyl)carbamoyl)-3- methylthiophen-2-yl)-2-(1- methyl-1H-pyrazol-4- yl)pyrazolo[5,1-b]thiazole-7- carboxamide |
| 36 | 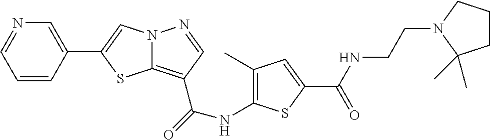 [SMILES: Cc1cc(C(=O)NCCN2CCCC2(C)C)sc1NC(=O)c1cnn2cc(-c3cccnc3)sc12] | N-(5-((2-(2,2- dimethylpyrrolidin-1- yl)ethyl)carbamoyl)-3- methylthiophen-2-yl)-2-(pyridin- 3-yl)pyrazolo[5,1-b]thiazole-7- carboxamide |
| 37 | 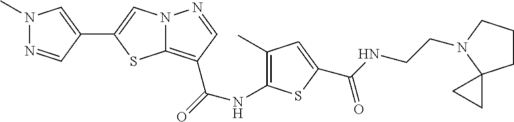 [SMILES: Cc1cc(C(=O)NCCN2CCCC23CC3)sc1NC(=O)c1cnn2cc(-c3cnn(C)c3)sc12] | N-(5-((2-(4-azaspiro[2.4]heptan- 4-yl)ethyl)carbamoyl)-3- methylthiophen-2-yl)-2-(1- methyl-1H-pyrazol-4- yl)pyrazolo[5,1-b]thiazole-7- carboxamide |
| 38 | 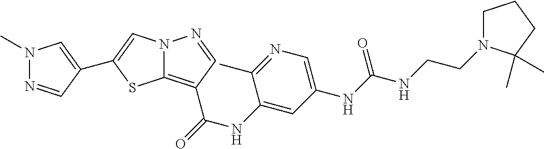 | N-(5-(3-(2-(2,2- dimethylpyrrolidin-1- yl)ethyl)ureido)-2-methyl- pyridin-3-yl)-2-(1-methyl-1H- pyrazol-4-yl)pyrazolo[5,1- b]thiazole-7-carboxamide |
| 39 | 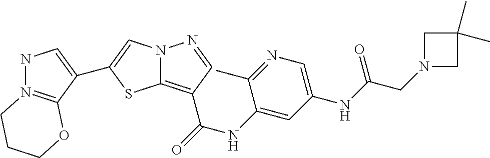 | 2-(6,7-dihydro-5H-pyrazolo[5,1- b][1,3]oxazin-3-yl)-N-(5-(2- (3,3-dimethylazetidin-1- yl)acetamido)-2-methylpyridin- 3-yl)pyrazolo[5,1-b]thiazole-7- carboxamide |
| 40 | 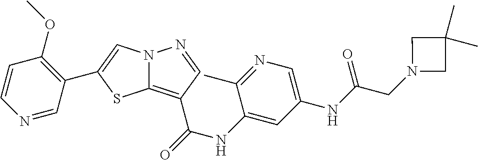 [SMILES: COc1ccncc1-c1cn2ncc(C(=O)Nc3cc(NC(=O)CN4CC(C)(C)C4)cnc3C)c2s1] | N-(5-(2-(3,3-dimethylazetidin-1- yl)acetamido)-2-methylpyridin- 3-yl)-2-(4-methoxypyridin-3- yl)pyrazolo[5,1-b]thiazole-7- carboxamide |
| 41 | 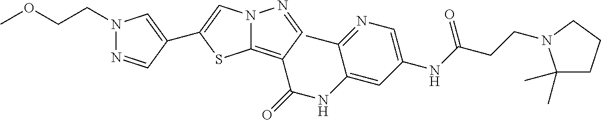 [SMILES: COCCn1cc(-c2cn3ncc(C(=O)Nc4cc(NC(=O)CCN5CCCC5(C)C)cnc4C)c3s2)cn1] | N-(5-(3-(2,2-dimethylpyrrolidin- 1-yl)propanamido)-2-methyl pyridin-3-yl)-2-(1-(2- methoxyethyl)-1H-pyrazol-4- yl)pyrazolo[5,1-b]thiazole-7- carboxamide |
| 42 | 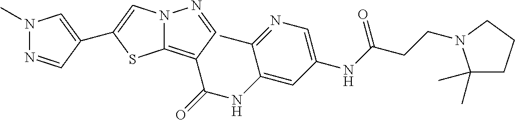 [SMILES: Cc1ncc(NC(=O)CCN2CCCC2(C)C)cc1NC(=O)c1cnn2cc(-c3cnn(C)c3)sc12] | N-(5-(3-(2,2-dimethylpyrrolidin- 1-yl)propanamido)-2-methyl- pyridin-3-yl)-2-(1-methyl-1H- pyrazol-4-yl)pyrazolo[5,1- b]thiazole-7-carboxamide |
| 43 | 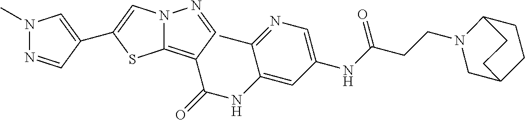 [SMILES: Cc1ncc(NC(=O)CCN2CC3CCC2CC3)cc1NC(=O)c1cnn2cc(-c3cnn(C)c3)sc12] | N-(5-(3-(2- azabicyclo[2.2.2]octan-2- yl)propanamido)-2-methyl pyridin-3-yl)-2-(1-methyl-1H- pyrazol-4-yl)pyrazolo[5,1- b]thiazole-7-carboxamide |
| 44 | 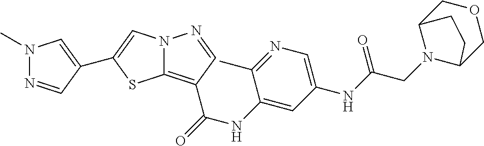 [SMILES: Cc1ncc(NC(=O)CN2C3CCC2COC3)cc1NC(=O)c1cnn2cc(-c3cnn(C)c3)sc12] | N-(5-(2-(3-oxa-8- azabicyclo[3.2.1]octan-8- yl)acetamido)-2-methylpyridin- 3-yl)-2-(1-methyl-1H-pyrazol-4- yl)pyrazolo[5,1-b]thiazole-7- carboxamide |
| 45 | 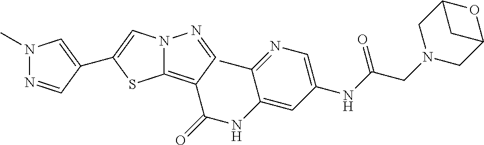 [SMILES: Cc1ncc(NC(=O)CN2CC3CC(C2)O3)cc1NC(=O)c1cnn2cc(-c3cnn(C)c3)sc12] | N-(5-(2-(6-oxa-3- azabicyclo[3.1.1]heptan-3- yl)acetamido)-2-methylpyridin- 3-yl)-2-(1-methyl-1H-pyrazol-4- yl)pyrazolo[5,1-b]thiazole-7- carboxamide |
| 46 | 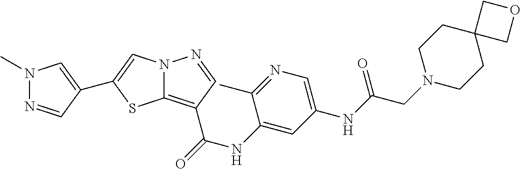 [SMILES: Cc1ncc(NC(=O)CN2CCC3(CC2)COC3)cc1NC(=O)c1cnn2cc(-c3cnn(C)c3)sc12] | N-(5-(2-(2-oxa-7- azaspiro[3.5]nonan-7- yl)acetamido)-2-methylpyridin- 3-yl)-2-(1-methyl-1H-pyrazol-4- yl)pyrazolo[5,1-b]thiazole-7- carboxamide |
| 47 | 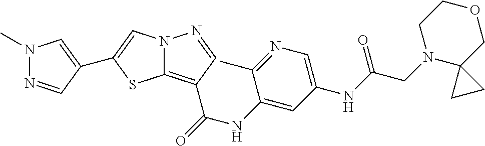 [SMILES: Cc1ncc(NC(=O)CN2CCOCC23CC3)cc1NC(=O)c1cnn2cc(-c3cnn(C)c3)sc12] | N-(5-(2-(7-oxa-4- azaspiro[2.5]octan-4- yl)acetamido)-2-methylpyridin- 3-yl)-2-(1-methyl-1H-pyrazol-4- yl)pyrazolo[5,1-b]thiazole-7- carboxamide |
| 48 | 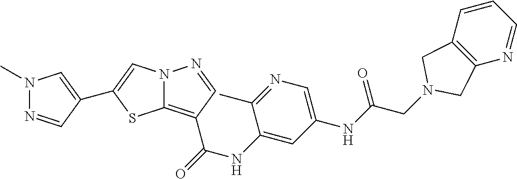 [SMILES: Cc1ncc(NC(=O)CN2Cc3cccnc3C2)cc1NC(=O)c1cnn2cc(-c3cnn(C)c3)sc12] | [00516] N-(5-(2-(5H- pyrrolo[3,4-b]pyridin-6(7H)- yl)acetamido)-2-methylpyridin- 3-yl)-2-(1-methyl-1H-pyrazol-4- yl)pyrazolo[5,1-b]thiazole-7- carboxamide |
| 49 | 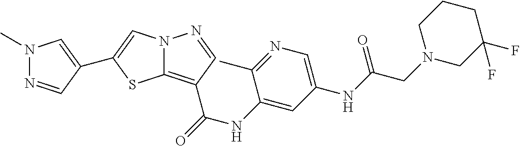 [SMILES: Cc1ncc(NC(=O)CN2CCCC(F)(F)C2)cc1NC(=O)c1cnn2cc(-c3cnn(C)c3)sc12] | [00517] N-(5-(2-(3,3- difluoropiperidin-1- yl)acetamido)-2-methylpyridin- 3-yl)-2-(1-methyl-1H-pyrazol-4- yl)pyrazolo[5,1-b]thiazole-7- carboxamide |
| 50 | 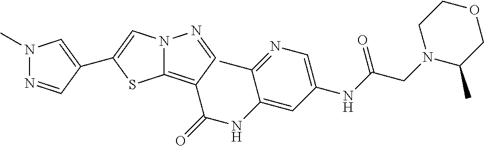 [SMILES: Cc1ncc(NC(=O)CN2CCOC[C@H]2C)cc1NC(=O)c1cnn2cc(-c3cnn(C)c3)sc12] | (R)-2-(1-methyl-1H-pyrazol-4- yl)-N-(2-methyl-5-(2-(3- methylmorpholino)acetamido) pyridin-3-yl)pyrazolo[5,1- b]thiazole-7-carboxamide |
| 51 | 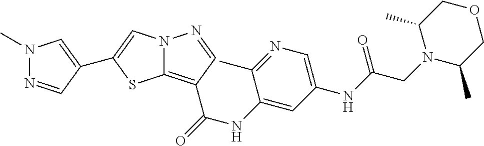 [SMILES: Cc1ncc(NC(=O)CN2[C@H](C)COC[C@H]2C)cc1NC(=O)c1cnn2cc(-c3cnn(C)c3)sc12] | N-(5-(2-((3R,5R)-3,5- dimethylmorpholino)acetamido)- 2-methylpyridin-3-yl)-2-(1- methyl-1H-pyrazol-4- yl)pyrazolo[5,1-b]thiazole-7- carboxamide |
| 52 | 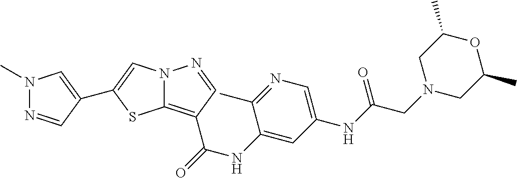 | N-(5-(2-((2S,6S)-2,6- dimethylmorpholino)acetamido)- 2-methylpyridin-3-yl)-2-(1- methyl-1H-pyrazol-4- yl)pyrazolo[5,1-b]thiazole-7- carboxamide |
| 53 | 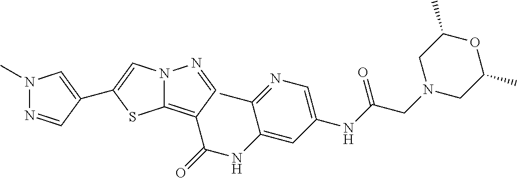 | N-(5-(2-((2R,6S)-2,6- dimethylmorpholino)acetamido)- 2-methylpyridin-3-yl)-2-(1- methyl-1H-pyrazol-4- yl)pyrazolo[5,1-b]thiazole-7- carboxamide |
| 54 | 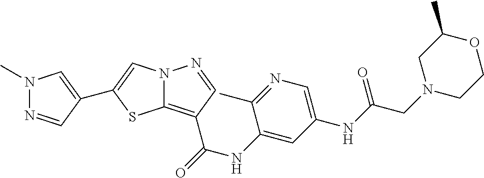 [SMILES: Cc1ncc(NC(=O)CN2CCO[C@H](C)C2)cc1NC(=O)c1cnn2cc(-c3cnn(C)c3)sc12] | (R)-2-(1-methyl-1H-pyrazol-4- yl)-N-(2-methyl-5-(2-(2- methylmorpholino)acetamido) pyridin-3-yl)pyrazolo[5,1- b]thiazole-7-carboxamide |
| 55 | 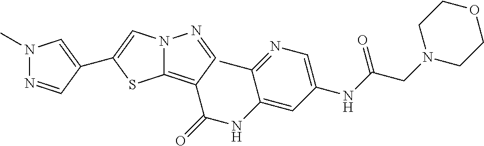 [SMILES: Cc1ncc(NC(=O)CN2CCOCC2)cc1NC(=O)c1cnn2cc(-c3cnn(C)c3)sc12] | 2-(1-methyl-1H-pyrazol-4-yl)- N-(2-methyl-5-(2- morpholinoacetamido)pyridin-3- yl)pyrazolo[5,1-b]thiazole-7- carboxamide |
| 57 | 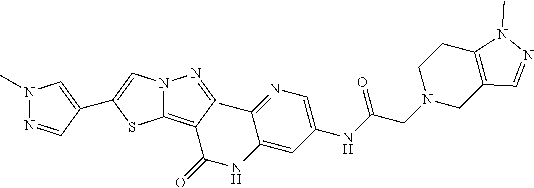 [SMILES: Cc1ncc(NC(=O)CN2CCc3c(cnn3C)C2)cc1NC(=O)c1cnn2cc(-c3cnn(C)c3)sc12] | 2-(1-methyl-1H-pyrazol-4-yl)- N-(2-methyl-5-(2-(1-methyl-6,7- dihydro-1H-pyrazolo[4,3- c]pyridin-5(4H)- yl)acetamido)pyridin-3- yl)pyrazolo[5,1-b]thiazole-7- carboxamide |
| 58 | 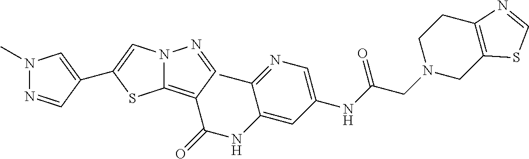 [SMILES: Cc1ncc(NC(=O)CN2CCc3ncsc3C2)cc1NC(=O)c1cnn2cc(-c3cnn(C)c3)sc12] | N-(5-(2-(6,7- dihydrothiazolo[5,4-c]pyridin- 5(4H)-yl)acetamido)-2- methylpyridin-3-yl)-2-(1- methyl-1H-pyrazol-4- yl)pyrazolo[5,1-b]thiazole-7- carboxamide |
| 59 | 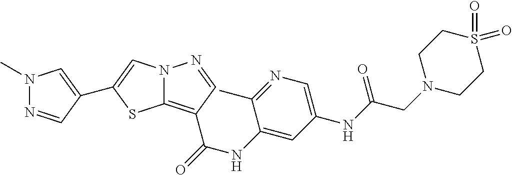 [SMILES: Cc1ncc(NC(=O)CN2CCS(=O)(=O)CC2)cc1NC(=O)c1cnn2cc(-c3cnn(C)c3)sc12] | N-(5-(2-(1,1- dioxidothiomorpholino)acetamido)- 2-methylpyridin-3-yl)-2-(1- methyl-1H-pyrazol-4- yl)pyrazolo[5,1-b]thiazole-7- carboxamide |
| 60 | 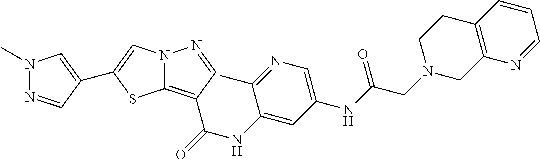 [SMILES: Cc1ncc(NC(=O)CN2CCc3cccnc3C2)cc1NC(=O)c1cnn2cc(-c3cnn(C)c3)sc12] | N-(5-(2-(5,6-dihydro-1,7- naphthyridin-7(8H)- yl)acetamido)-2-methylpyridin- 3-yl)-2-(1-methyl-1H-pyrazol-4- yl)pyrazolo[5,1-b]thiazole-7- carboxamide |
| 61 | 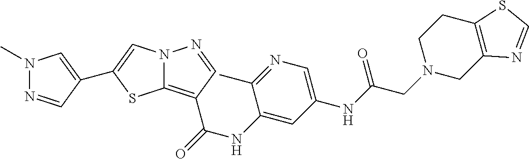 [SMILES: Cc1ncc(NC(=O)CN2CCc3scnc3C2)cc1NC(=O)c1cnn2cc(-c3cnn(C)c3)sc12] | N-(5-(2-(6,7- dihydrothiazolo[4,5-c]pyridin- 5(4H)-yl)acetamido)-2- methylpyridin-3-yl)-2-(1- methyl-1H-pyrazol-4- yl)pyrazolo[5,1-b]thiazole-7- carboxamide |
| 62 | 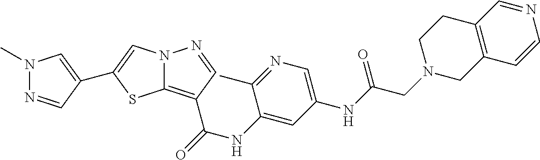 [SMILES: Cc1ncc(NC(=O)CN2CCc3cnccc3C2)cc1NC(=O)c1cnn2cc(-c3cnn(C)c3)sc12] | N-(5-(2-(3,4-dihydro-2,6- naphthyridin-2(1H)- yl)acetamido)-2-methylpyridin- 3-yl)-2-(1-methyl-1H-pyrazol-4- yl)pyrazolo[5,1-b]thiazole-7- carboxamide |
| 63 | 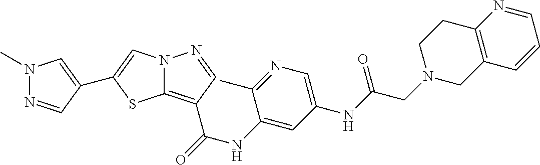 [SMILES: Cc1ncc(NC(=O)CN2CCc3ncccc3C2)cc1NC(=O)c1cnn2cc(-c3cnn(C)c3)sc12] | N-(5-(2-(7,8-dihydro-1,6- naphthyridin-6(5H)- yl)acetamido)-2-methylpyridin- 3-yl)-2-(1-methyl-1H-pyrazol-4- yl)pyrazolo[5,1-b]thiazole-7- carboxamide |
| 64 | 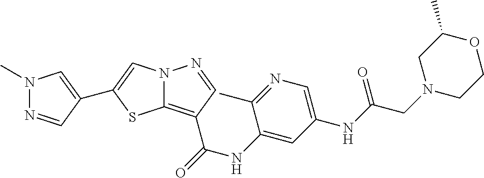 [SMILES: Cc1ncc(NC(=O)CN2CCO[C@@H](C)C2)cc1NC(=O)c1cnn2cc(-c3cnn(C)c3)sc12] | (S)-2-(1-methyl-1H-pyrazol-4- yl)-N-(2-methyl-5-(2-(2- methylmorpholino)acetamido) pyridin-3-yl)pyrazolo[5,1- b]thiazole-7-carboxamide |
| 65 | 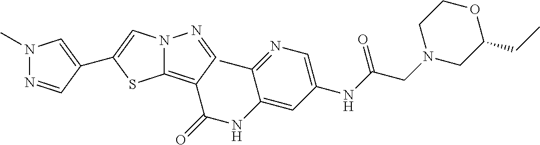 [SMILES: CC[C@@H]1CN(CC(=O)Nc2cnc(C)c(NC(=O)c3cnn4cc(-c5cnn(C)c5)sc34)c2)CCO1] | (S)-2-(1-methyl-1H (R)-N-(5-(2- (2-ethylmorpholino)acetamido)- 2-methylpyridin-3-yl)-2-(1- methyl-1H-pyrazol-4- yl)pyrazolo[5,1-b]thiazole-7- carboxamide |
| 66 | 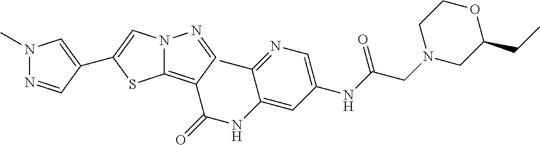 [SMILES: CC[C@H]1CN(CC(=O)Nc2cnc(C)c(NC(=O)c3cnn4cc(-c5cnn(C)c5)sc34)c2)CCO1] | (S)-N-(5-(2-(2- ethylmorpholino)acetamido)-2- methylpyridin-3-yl)-2-(1- methyl-1H-pyrazol-4- yl)pyrazolo[5,1-b]thiazole-7- carboxamide |
| 67 | 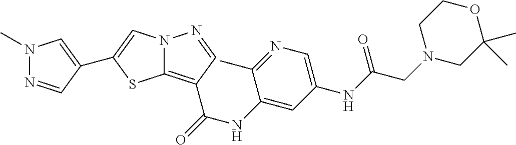 | N-(5-(2-(2,2- dimethylmorpholino)acetamido)- 2-methylpyridin-3-yl)-2-(1- methyl-1H-pyrazol-4- yl)pyrazolo[5,1-b]thiazole-7- carboxamide |
| 68 | 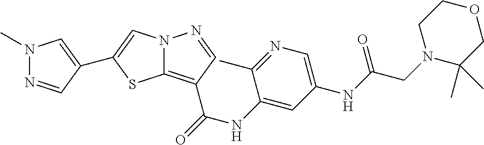 | N-(5-(2-(3,3- dimethylmorpholino)acetamido)- 2-methylpyridin-3-yl)-2-(1- methyl-1H-pyrazol-4- yl)pyrazolo[5,1-b]thiazole-7- carboxamide |
| 69 | 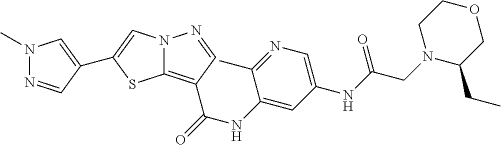 [SMILES: CC[C@@H]1COCCN1CC(=O)Nc1cnc(C)c(NC(=O)c2cnn3cc(-c4cnn(C)c4)sc23)c1] | (R)-N-(5-(2-(3- ethylmorpholino)acetamido)-2- methylpyridin-3-yl)-2-(1- methyl-1H-pyrazol-4- yl)pyrazolo[5,1-b]thiazole-7- carboxamide |
| 70 | 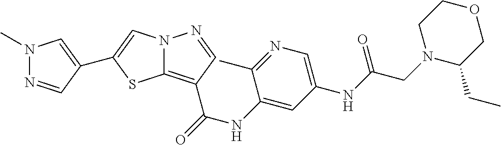 [SMILES: CC[C@H]1COCCN1CC(=O)Nc1cnc(C)c(NC(=O)c2cnn3cc(-c4cnn(C)c4)sc23)c1] | (S)-N-(5-(2-(3- ethylmorpholino)acetamido)-2- methylpyridin-3-yl)-2-(1- methyl-1H-pyrazol-4- yl)pyrazolo[5,1-b]thiazole-7- carboxamide |
| 71 | 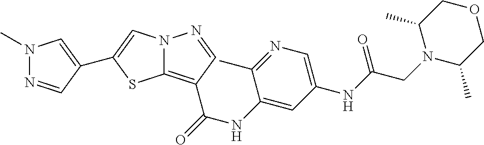 [SMILES: Cc1ncc(NC(=O)CN2[C@H](C)COC[C@@H]2C)cc1NC(=O)c1cnn2cc(-c3cnn(C)c3)sc12] | N-(5-(2-((3S,5R)-3,5- dimethylmorpholino)acetamido)- 2-methylpyridin-3-yl)-2-(1- methyl-1H-pyrazol-4- yl)pyrazolo[5,1-b]thiazole-7- carboxamide |
| 72 | 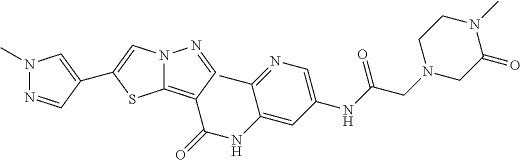 [SMILES: Cc1ncc(NC(=O)CN2CCN(C)C(=O)C2)cc1NC(=O)c1cnn2cc(-c3cnn(C)c3)sc12] | 2-(1-methyl-1H-pyrazol-4-yl)- N-(2-methyl-5-(2-(4-methyl-3- oxopiperazin-1- yl)acetamido)pyridin-3- yl)pyrazolo[5,1-b]thiazole-7- carboxamide |
| 73 | 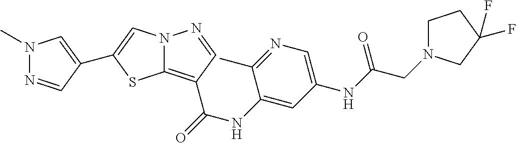 [SMILES: Cc1ncc(NC(=O)CN2CCC(F)(F)C2)cc1NC(=O)c1cnn2cc(-c3cnn(C)c3)sc12] | N-(5-(2-(3,3-difluoropyrrolidin- 1-yl)acetamido)-2- methylpyridin-3-yl)-2-(1- methyl-1H-pyrazol-4- yl)pyrazolo[5,1-b]thiazole-7- carboxamide |
| 74 | 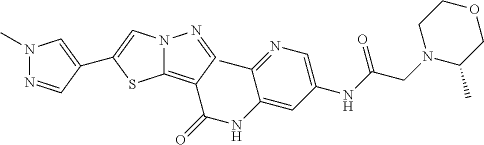 [SMILES: Cc1ncc(NC(=O)CN2CCOC[C@@H]2C)cc1NC(=O)c1cnn2cc(-c3cnn(C)c3)sc12] | (S)-2-(1-methyl-1H-pyrazol-4- yl)-N-(2-methyl-5-(2-(3- methylmorpholino)acetamido) pyridin-3-yl)pyrazolo[5,1- b]thiazole-7-carboxamide |
| 75 | 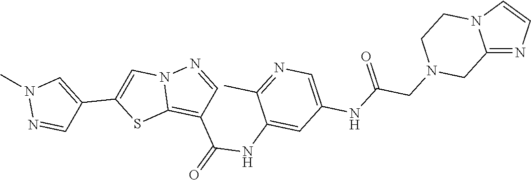 [SMILES: Cc1ncc(NC(=O)CN2CCn3ccnc3C2)cc1NC(=O)c1cnn2cc(-c3cnn(C)c3)sc12] | N-(5-(2-(5,6- dihydroimidazo[1,2-a]pyrazin- 7(8H)-yl)acetamido)-2- methylpyridin-3-yl)-2-(1- methyl-1H-pyrazol-4- yl)pyrazolo[5,1-b]thiazole-7- carboxamide |
| 76 | 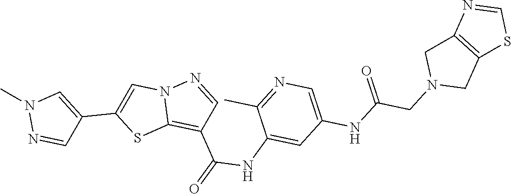 [SMILES: Cc1ncc(NC(=O)CN2Cc3ncsc3C2)cc1NC(=O)c1cnn2cc(-c3cnn(C)c3)sc12] | N-(5-(2-(4H-pyrrolo[3,4- d]thiazol-5(6H)-yl)acetamido)- 2-methylpyridin-3-yl)-2-(1- methyl-1H-pyrazol-4- yl)pyrazolo[5,1-b]thiazole-7- carboxamide |
| 77 | 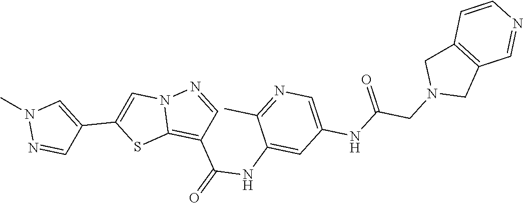 [SMILES: Cc1ncc(NC(=O)CN2Cc3ccncc3C2)cc1NC(=O)c1cnn2cc(-c3cnn(C)c3)sc12] | N-(5-(2-(1H-pyrrolo[3,4- c]pyridin-2(3H)-yl)acetamido)- 2-methylpyridin-3-yl)-2-(1- methyl-1H-pyrazol-4- yl)pyrazolo[5,1-b]thiazole-7- carboxamide |
| 78 | 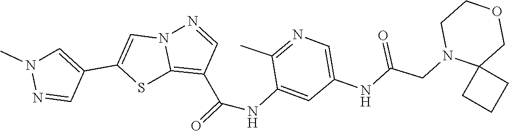 [SMILES: Cc1ncc(NC(=O)CN2CCOCC23CCC3)cc1NC(=O)c1cnn2cc(-c3cnn(C)c3)sc12] | N-(5-(2-(8-oxa-5- azaspiro[3.5]nonan-5- yl)acetamido)-2-methylpyridin- 3-yl)-2-(1-methyl-1H-pyrazol-4- yl)pyrazolo[5,1-b]thiazole-7- carboxamide |
| 79 | 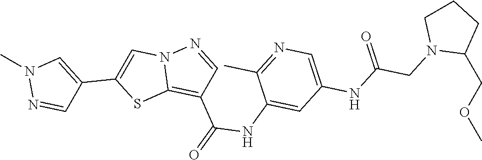 | N-(5-(2-(2- (methoxymethyl)pyrrolidin-1- yl)acetamido)-2-methylpyridin- 3-yl)-2-(1-methyl-1H-pyrazol-4- yl)pyrazolo[5,1-b]thiazole-7- carboxamide |
| 80 | 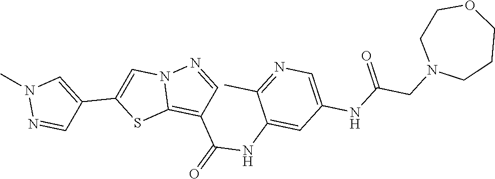 [SMILES: Cc1ncc(NC(=O)CN2CCCOCC2)cc1NC(=O)c1cnn2cc(-c3cnn(C)c3)sc12] | N-(5-(2-(1,4-oxazepan-4- yl)acetamido)-2-methylpyridin- 3-yl)-2-(1-methyl-1H-pyrazol-4- yl)pyrazolo[5,1-b]thiazole-7- carboxamide |
| 81 | 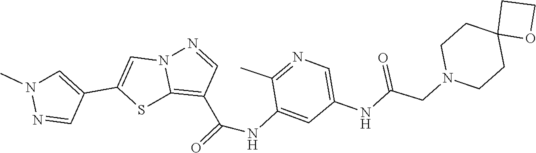 [SMILES: Cc1ncc(NC(=O)CN2CCC3(CCO3)CC2)cc1NC(=O)c1cnn2cc(-c3cnn(C)c3)sc12] | N-(5-(2-(1-oxa-7- azaspiro[3.5]nonan-7- yl)acetamido)-2-methylpyridin- 3-yl)-2-(1-methyl-1H-pyrazol-4- yl)pyrazolo[5,1-b]thiazole-7- carboxamide |
| 82 | 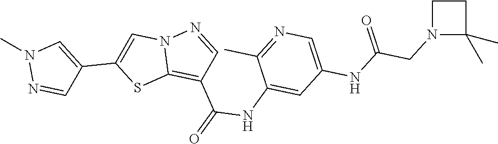 | N-(5-(2-(2,2-dimethylazetidin-1- yl)acetamido)-2-methylpyridin- 3-yl)-2-(1-methyl-1H-pyrazol-4- yl)pyrazolo[5,1-b]thiazole-7- carboxamide |
| 83 | 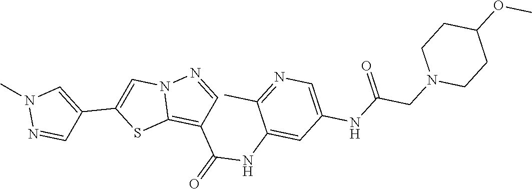 | N-(5-(2-(4-methoxypiperidin-1- yl)acetamido)-2-methylpyridin- 3-yl)-2-(1-methyl-1H-pyrazol-4- yl)pyrazolo[5,1-b]thiazole-7- carboxamide |
| 84 | 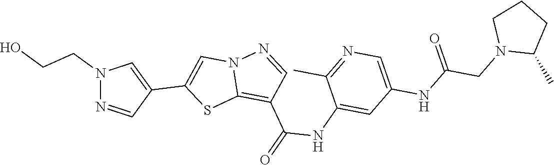 [SMILES: Cc1ncc(NC(=O)CN2CCC[C@@H]2C)cc1NC(=O)c1cnn2cc(-c3cnn(CCO)c3)sc12] | (S)-2-(1-(2- hydroxyethyl)-1H-pyrazol-4-yl)- N-(2-methyl-5-(2-(2- methylpyrrolidin-1- yl)acetamido)pyridin-3- yl)pyrazolo[5,1-b]thiazole-7- carboxamide |
| 85 | 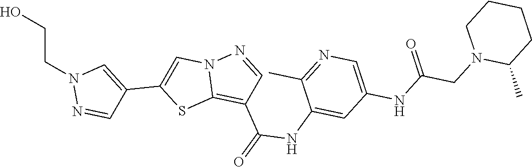 [SMILES: Cc1ncc(NC(=O)CN2CCCC[C@@H]2C)cc1NC(=O)c1cnn2cc(-c3cnn(CCO)c3)sc12] | (S)-2-(1-(2-hydroxyethyl)-1H- pyrazol-4-yl)-N-(2-methyl-5-(2- (2-methylpiperidin-1- yl)acetamido)pyridin-3- yl)pyrazolo[5,1-b]thiazole-7- carboxamide |
| 86 | 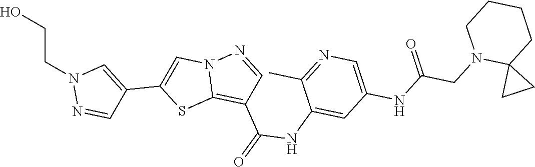 [SMILES: Cc1ncc(NC(=O)CN2CCCCC23CC3)cc1NC(=O)c1cnn2cc(-c3cnn(CCO)c3)sc12] | N-(5-(2-(4-azaspiro[2.5]octan-4- yl)acetamido)-2-methylpyridin- 3-yl)-2-(1-(2-hydroxyethyl)-1H- pyrazol-4-yl)pyrazolo[5,1- b]thiazole-7-carboxamide |
| 87 | 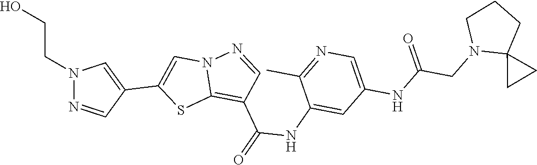 [SMILES: Cc1ncc(NC(=O)CN2CCCC23CC3)cc1NC(=O)c1cnn2cc(-c3cnn(CCO)c3)sc12] | N-(5-(2-(4-azaspiro[2.4]heptan- 4-yl)acetamido)-2- methylpyridin-3-yl)-2-(1-(2- hydroxyethyl)-1H-pyrazol-4- yl)pyrazolo[5,1-b]thiazole-7- carboxamide |
| 88 | 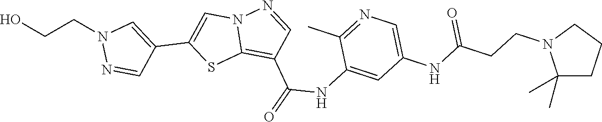 [SMILES: Cc1ncc(NC(=O)CCN2CCCC2(C)C)cc1NC(=O)c1cnn2cc(-c3cnn(CCO)c3)sc12] | N-(5-(3-(2,2-dimethylpyrrolidin- 1-yl)propanamido)-2- methylpyridin-3-yl)-2-(1-(2- hydroxyethyl)-1H-pyrazol-4- yl)pyrazolo[5,1-b]thiazole-7- carboxamide |
| 89 | 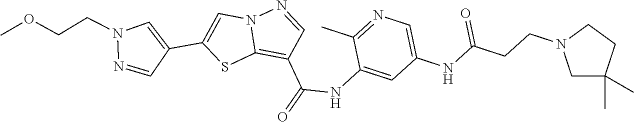 | N-(5-(3-(3,3-dimethylpyrrolidin- 1-yl)propanamido)-2- methylpyridin-3-yl)-2-(1-(2- methoxyethyl)-1H-pyrazol-4- yl)pyrazolo[5,1-b]thiazole-7- carboxamide |
| 90 | 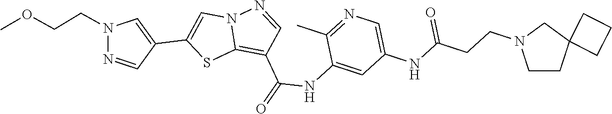 [SMILES: COCCn1cc(-c2cn3ncc(C(=O)Nc4cc(NC(=O)CCN5CCC6(CCC6)C5)cnc4C)c3s2)cn1] | N-(5-(3-(6-azaspiro[3.4]octan-6- yl)propanamido)-2- methylpyridin-3-yl)-2-(1-(2- methoxyethyl)-1H-pyrazol-4- yl)pyrazolo[5,1-b]thiazole-7- carboxamide |
| 91 | 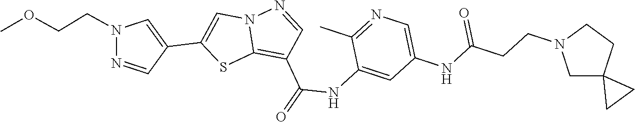 [SMILES: COCCn1cc(-c2cn3ncc(C(=O)Nc4cc(NC(=O)CCN5CCC6(CC6)C5)cnc4C)c3s2)cn1] | N-(5-(3-(5-azaspiro[2.4]heptan- 5-yl)propanamido)-2- methylpyridin-3-yl)-2-(1-(2- methoxyethyl)-1H-pyrazol-4- yl)pyrazolo[5,1-b]thiazole-7- carboxamide |
| 92 | 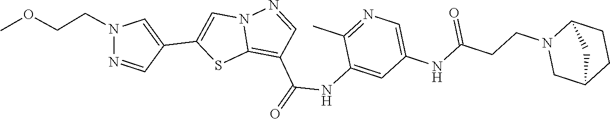 [SMILES: COCCn1cc(-c2cn3ncc(C(=O)Nc4cc(NC(=O)CCN5C[C@@H]6CC[C@H]5C6)cnc4C)c3s2)cn1] | N-(5-(3-((1S,4R)-2- azabicyclo[2.2.1]heptan-2- yl)propanamido)-2- methylpyridin-3-yl)-2-(1-(2- methoxyethyl)-1H-pyrazol-4- yl)pyrazolo[5,1-b]thiazole-7- carboxamide |
| 93 | 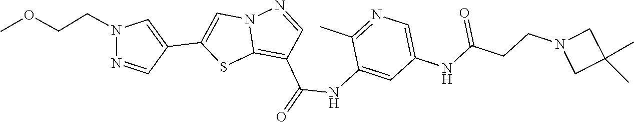 | N-(5-(3-(3,3-dimethylazetidin-1- yl)propanamido)-2- methylpyridin-3-yl)-2-(1-(2- methoxyethyl)-1H-pyrazol-4- yl)pyrazolo[5,1-b]thiazole-7- carboxamide |
| 94 | 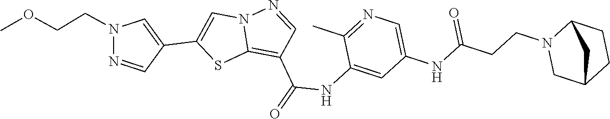 [SMILES: COCCn1cc(-c2cn3ncc(C(=O)Nc4cc(NC(=O)CCN5C[C@H]6CC[C@@H]5C6)cnc4C)c3s2)cn1] | N-(5-(3-((1R,4S)-2- azabicyclo[2.2.1]heptan-2- yl)propanamido)-2- methylpyridin-3-yl)-2-(1-(2- methoxyethyl)-1H-pyrazol-4- yl)pyrazolo[5,1-b]thiazole-7- carboxamide |
| 95 | 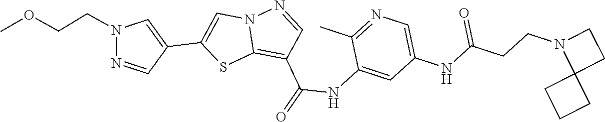 [SMILES: COCCn1cc(-c2cn3ncc(C(=O)Nc4cc(NC(=O)CCN5CCC56CCC6)cnc4C)c3s2)cn1] | N-(5-(3-(1-azaspiro[3.3]heptan- 1-yl)propanamido)-2- methylpyridin-3-yl)-2-(1-(2- methoxyethyl)-1H-pyrazol-4- yl)pyrazolo[5,1-b]thiazole-7- carboxamide |
| 96 | 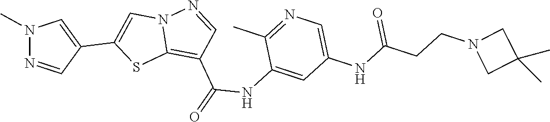 | N-(5-(3-(3,3-dimethylazetidin-1- yl)propanamido)-2- methylpyridin-3-yl)-2-(1- methyl-1H-pyrazol-4- yl)pyrazolo[5,1-b]thiazole-7- carboxamide |
| 97 | 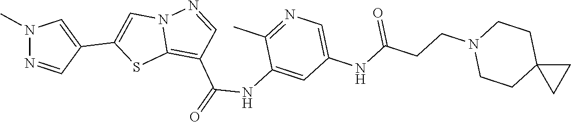 [SMILES: Cc1ncc(NC(=O)CCN2CCC3(CC2)CC3)cc1NC(=O)c1cnn2cc(-c3cnn(C)c3)sc12] | N-(5-(3-(6-azaspiro[2.5]octan-6- yl)propanamido)-2- methylpyridin-3-yl)-2-(1- methyl-1H-pyrazol-4- yl)pyrazolo[5,1-b]thiazole-7- carboxamide |
| 98 | 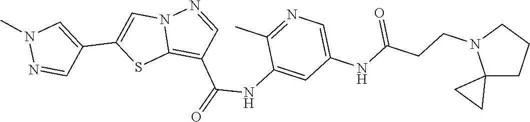 [SMILES: Cc1ncc(NC(=O)CCN2CCCC23CC3)cc1NC(=O)c1cnn2cc(-c3cnn(C)c3)sc12] | N-(5-(3-(2- azabicyclo[2.2.2]octan-2- yl)propanamido)-2- methylpyridin-3-yl)-2-(1-(2- methoxyethyl)-1H-pyrazol-4- yl)pyrazolo[5,1-b]thiazole-7- carboxamide |
| 100 | 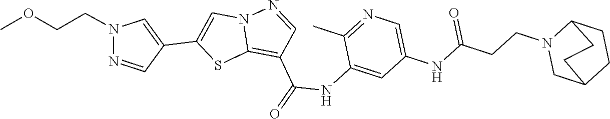 [SMILES: COCCn1cc(-c2cn3ncc(C(=O)Nc4cc(NC(=O)CCN5CC6CCC5CC6)cnc4C)c3s2)cn1] | N-(5-(3-(2- azabicyclo[2.2.2]octan-2- yl)propanamido)-2- methylpyridin-3-yl)-2-(1-(2- methoxyethyl)-1H-pyrazol-4- yl)pyrazolo[5,1-b]thiazole-7- carboxamide |
| 102 | 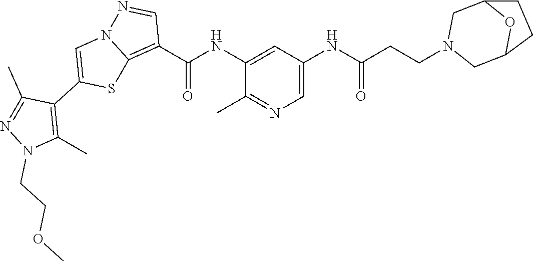 [SMILES: COCCn1nc(C)c(-c2cn3ncc(C(=O)Nc4cc(NC(=O)CCN5CC6CCC(C5)O6)cnc4C)c3s2)c1C] | N-(5-(3-(8-oxa-3- azabicyclo[3.2.1]octan-3- yl)propanamido)-2- methylpyridin-3-yl)-2-(1-(2- methoxyethyl)-3,5-dimethyl-1H- pyrazol-4-yl)pyrazolo[5,1- b]thiazole-7-carboxamide |
| 103 | 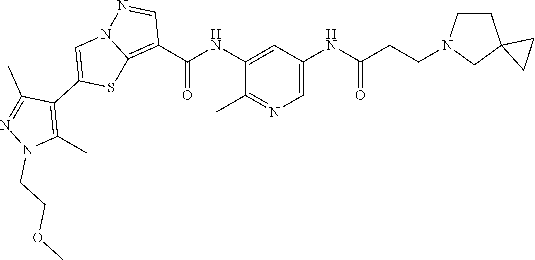 [SMILES: COCCn1nc(C)c(-c2cn3ncc(C(=O)Nc4cc(NC(=O)CCN5CCC6(CC6)C5)cnc4C)c3s2)c1C] | N-(5-(3-(5-azaspiro[2.4]heptan- 5-yl)propanamido)-2- methylpyridin-3-yl)-2-(1-(2- methoxyethyl)-3,5-dimethyl-1H- pyrazol-4-yl)pyrazolo[5,1- b]thiazole-7-carboxamide |
| 104 | 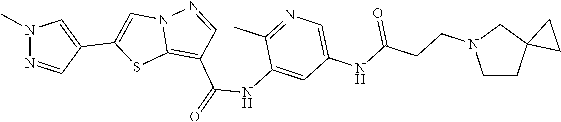 [SMILES: Cc1ncc(NC(=O)CCN2CCC3(CC3)C2)cc1NC(=O)c1cnn2cc(-c3cnn(C)c3)sc12] | N-(5-(3-(5-azaspiro[2.4]heptan- 5-yl)propanamido)-2- methylpyridin-3-yl)-2-(1- methyl-1H-pyrazol-4- yl)pyrazolo[5,1-b]thiazole-7- carboxamide |
| 106 | 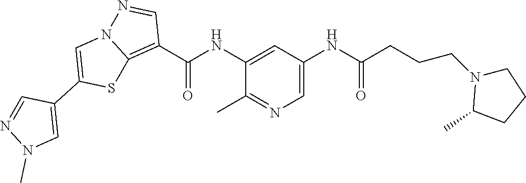 [SMILES: Cc1ncc(NC(=O)CCCN2CCC[C@@H]2C)cc1NC(=O)c1cnn2cc(-c3cnn(C)c3)sc12] | (S)-2-(1-methyl-1H-pyrazol-4- yl)-N-(2-methyl-5-(4-(2- methylpyrrolidin-1- yl)butanamido)pyridin-3- yl)pyrazolo[5,1-b]thiazole-7- carboxamide |
| 107 | 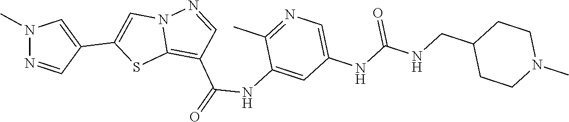 | 2-(1-methyl-1H-pyrazol-4-yl)- N-(2-methyl-5-(3-((1- methylpiperidin-4- yl)methyl)ureido)pyridin-3- yl)pyrazolo[5,1-b]thiazole-7- carboxamide |
| 108 | 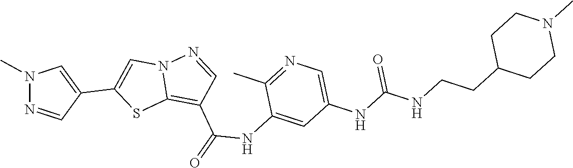 | 2-(1-methyl-1H-pyrazol-4-yl)- N-(2-methyl-5-(3-(2-(1- methylpiperidin-4- yl)ethyl)ureido)pyridin-3- yl)pyrazolo[5,1-b]thiazole-7- carboxamide |
| 109 | 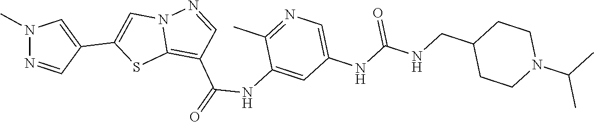 [SMILES: Cc1ncc(NC(=O)NCC2CCN(C(C)C)CC2)cc1NC(=O)c1cnn2cc(-c3cnn(C)c3)sc12] | N-(5-(3-((1-isopropylpiperidin- 4-yl)methyl)ureido)-2- methylpyridin-3-yl)-2-(1- methyl-1H-pyrazol-4- yl)pyrazolo[5,1-b]thiazole-7- carboxamide |
| 110 | 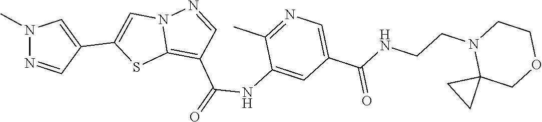 [SMILES: Cc1ncc(C(=O)NCCN2CCOCC23CC3)cc1NC(=O)c1cnn2cc(-c3cnn(C)c3)sc12] | N-(5-((2-(7-oxa-4- azaspiro[2.5]octan-4- yl)ethyl)carbamoyl)-2- methylpyridin-3-yl)-2-(1- methyl-1H-pyrazol-4- yl)pyrazolo[5,1-b]thiazole-7- carboxamide |
| 111 | 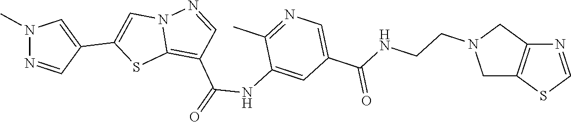 [SMILES: Cc1ncc(C(=O)NCCN2Cc3ncsc3C2)cc1NC(=O)c1cnn2cc(-c3cnn(C)c3)sc12] | N-(5-((2-(4H-pyrrolo[3,4- d]thiazol-5(6H)- yl)ethyl)carbamoyl)-2- methylpyridin-3-yl)-2-(1- methyl-1H-pyrazol-4- yl)pyrazolo[5,1-b]thiazole-7- carboxamide |
| 112 | 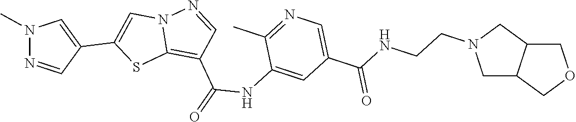 [SMILES: Cc1ncc(C(=O)NCCN2CC3COCC3C2)cc1NC(=O)c1cnn2cc(-c3cnn(C)c3)sc12] | 2-(1-methyl-1H-pyrazol-4-yl)- N-(2-methyl-5-((2-(tetrahydro- 1H-furo[3,4-c]pyrrol-5(3H)- yl)ethyl)carbamoyl)pyridin-3- yl)pyrazolo[5,1-b]thiazole-7- carboxamide |
| 113 | 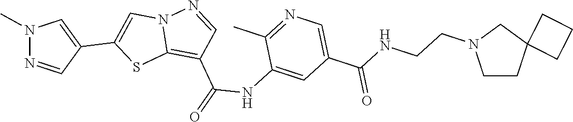 [SMILES: Cc1ncc(C(=O)NCCN2CCC3(CCC3)C2)cc1NC(=O)c1cnn2cc(-c3cnn(C)c3)sc12] | N-(5-((2-(6-azaspiro[3.4]octan- 6-yl)ethyl)carbamoyl)-2- methylpyridin-3-yl)-2-(1- methyl-1H-pyrazol-4- yl)pyrazolo[5,1-b]thiazole-7- carboxamide |
| 114 | 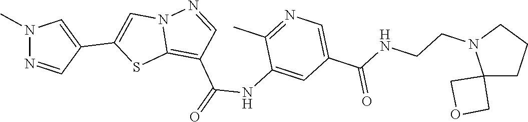 [SMILES: Cc1ncc(C(=O)NCCN2CCCC23COC3)cc1NC(=O)c1cnn2cc(-c3cnn(C)c3)sc12] | N-(5-((2-(2-oxa-5- azaspiro[3.4]octan-5- yl)ethyl)carbamoyl)-2- methylpyridin-3-yl)-2-(1- methyl-1H-pyrazol-4- yl)pyrazolo[5,1-b]thiazole-7- carboxamide |
| 115 | 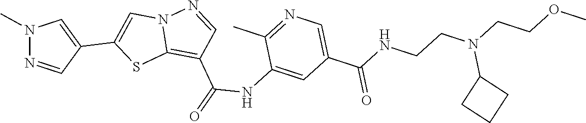 | N-(5-((2-(cyclobutyl(2- methoxyethyl)amino)ethyl) carbamoyl)- 2-methylpyridin-3-yl)-2- (1-methyl-1H-pyrazol-4- yl)pyrazolo[5,1-b]thiazole-7- carboxamide |
| 116 | 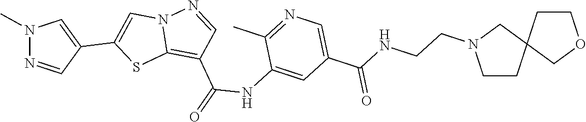 [SMILES: Cc1ncc(C(=O)NCCN2CCC3(CCOC3)C2)cc1NC(=O)c1cnn2cc(-c3cnn(C)c3)sc12] | N-(5-((2-(2-oxa-7- azaspiro[4.4]nonan-7- yl)ethyl)carbamoyl)-2- methylpyridin-3-yl)-2-(1- methyl-1H-pyrazol-4- yl)pyrazolo[5,1-b]thiazole-7- carboxamide |
| 117 | 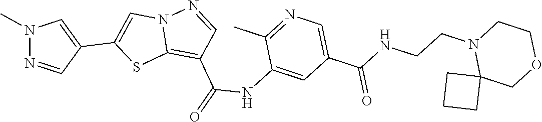 [SMILES: Cc1ncc(C(=O)NCCN2CCOCC23CCC3)cc1NC(=O)c1cnn2cc(-c3cnn(C)c3)sc12] | N-(5-((2-(8-oxa-5- azaspiro[3.5]nonan-5- yl)ethyl)carbamoyl)-2- methylpyridin-3-yl)-2-(1- methyl-1H-pyrazol-4- yl)pyrazolo[5,1-b]thiazole-7- carboxamide |
| 118 | 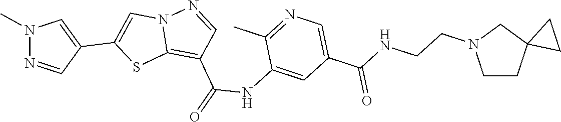 [SMILES: Cc1ncc(C(=O)NCCN2CCC3(CC3)C2)cc1NC(=O)c1cnn2cc(-c3cnn(C)c3)sc12] | N-(5-((2-(5-azaspiro[2.4]heptan- 5-yl)ethyl)carbamoyl)-2- methylpyridin-3-yl)-2-(1- methyl-1H-pyrazol-4- yl)pyrazolo[5,1-b]thiazole-7- carboxamide |
| 119 | 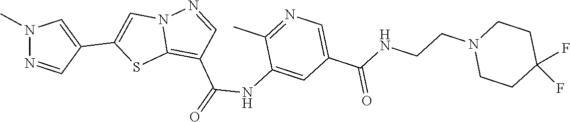 [SMILES: Cc1ncc(C(=O)NCCN2CCC(F)(F)CC2)cc1NC(=O)c1cnn2cc(-c3cnn(C)c3)sc12] | N-(5-((2-(4,4-difluoropiperidin- 1-yl)ethyl)carbamoyl)-2- methylpyridin-3-yl)-2-(1- methyl-1H-pyrazol-4- yl)pyrazolo[5,1-b]thiazole-7- carboxamide |
| 120 | 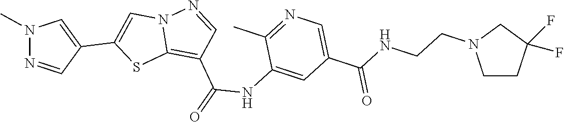 [SMILES: Cc1ncc(C(=O)NCCN2CCC(F)(F)C2)cc1NC(=O)c1cnn2cc(-c3cnn(C)c3)sc12] | N-(5-((2-(3,3-difluoropyrrolidin- 1-yl)ethyl)carbamoyl)-2- methylpyridin-3-yl)-2-(1- methyl-1H-pyrazol-4- yl)pyrazolo[5,1-b]thiazole-7- carboxamide |
| 121 | 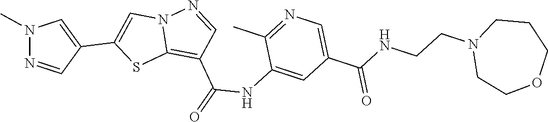 [SMILES: Cc1ncc(C(=O)NCCN2CCCOCC2)cc1NC(=O)c1cnn2cc(-c3cnn(C)c3)sc12] | N-(5-((2-(1,4-oxazepan-4- yl)ethyl)carbamoyl)-2- methylpyridin-3-yl)-2-(1- methyl-1H-pyrazol-4- yl)pyrazolo[5,1-b]thiazole-7- carboxamide |
| 122 | 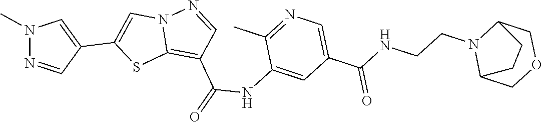 [SMILES: Cc1ncc(C(=O)NCCN2C3CCC2COC3)cc1NC(=O)c1cnn2cc(-c3cnn(C)c3)sc12] | N-(5-((2-(3-oxa-8- azabicyclo[3.2.1]octan-8- yl)ethyl)carbamoyl)-2- methylpyridin-3-yl)-2-(1- methyl-1H-pyrazol-4- yl)pyrazolo[5,1-b]thiazole-7- carboxamide |
| 123 | 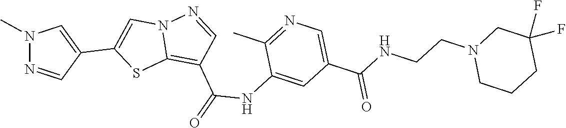 [SMILES: Cc1ncc(C(=O)NCCN2CCCC(F)(F)C2)cc1NC(=O)c1cnn2cc(-c3cnn(C)c3)sc12] | N-(5-((2-(3,3-difluoropiperidin- 1-yl)ethyl)carbamoyl)-2- methylpyridin-3-yl)-2-(1- methyl-1H-pyrazol-4- yl)pyrazolo[5,1-b]thiazole-7- carboxamide |
| 124 | 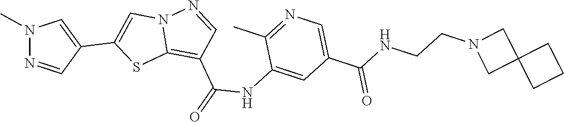 [SMILES: Cc1ncc(C(=O)NCCN2CC3(CCC3)C2)cc1NC(=O)c1cnn2cc(-c3cnn(C)c3)sc12] | N-(5-((2-(2-azaspiro[3.3]heptan- 2-yl)ethyl)carbamoyl)-2- methylpyridin-3-yl)-2-(1- methyl-1H-pyrazol-4- yl)pyrazolo[5,1-b]thiazole-7- carboxamide |
| 125 | 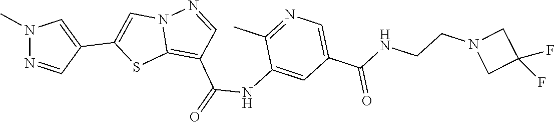 [SMILES: Cc1ncc(C(=O)NCCN2CC(F)(F)C2)cc1NC(=O)c1cnn2cc(-c3cnn(C)c3)sc12] | N-(5-((2-(3,3-difluoroazetidin-1- yl)ethyl)carbamoyl)-2- methylpyridin-3-yl)-2-(1- methyl-1H-pyrazol-4- yl)pyrazolo[5,1-b]thiazole-7- carboxamide |
| 126 | 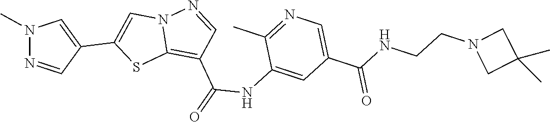 | N-(5-((2-(3,3-dimethylazetidin- 1-yl)ethyl)carbamoyl)-2- methylpyridin-3-yl)-2-(1- methyl-1H-pyrazol-4- yl)pyrazolo[5,1-b]thiazole-7- carboxamide |
| 127 | 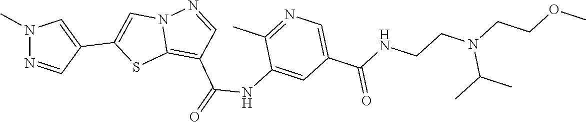 | N-(5-((2-(isopropyl(2- methoxyethyl)amino)ethyl) carbamoyl)- 2-methylpyridin-3-yl)-2- (1-methyl-1H-pyrazol-4- yl)pyrazolo[5,1-b]thiazole-7- carboxamide |
| 128 | 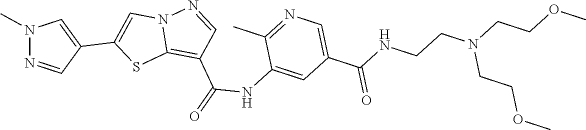 | N-(5-((2-(bis(2- methoxyethyl)amino)ethyl) carbamoyl)-2-methylpyridin- 3-yl)-2- (1-methyl-1H-pyrazol-4- yl)pyrazolo[5,1-b]thiazole-7- carboxamide |
| 129 | 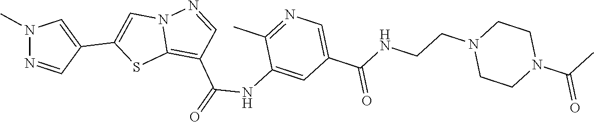 [SMILES: CC(=O)N1CCN(CCNC(=O)c2cnc(C)c(NC(=O)c3cnn4cc(-c5cnn(C)c5)sc34)c2)CC1] | N-(5-((2-(4-acetylpiperazin-1- yl)ethyl)carbamoyl)-2- methylpyridin-3-yl)-2-(1- methyl-1H-pyrazol-4- yl)pyrazolo[5,1-b]thiazole-7- carboxamide |
| 130 | 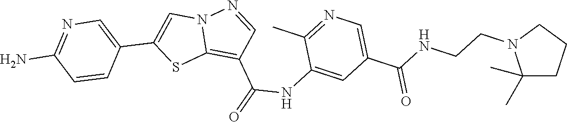 [SMILES: Cc1ncc(C(=O)NCCN2CCCC2(C)C)cc1NC(=O)c1cnn2cc(-c3ccc(N)nc3)sc12] | 2-(6-aminopyridin-3-yl)-N-(5- ((2-(2,2-dimethylpyrrolidin-1- yl)ethyl)carbamoyl)-2- methylpyridin-3- yl)pyrazolo[5,1-b]thiazole-7- carboxamide |
| 131 | 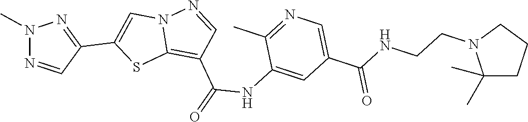 [SMILES: Cc1ncc(C(=O)NCCN2CCCC2(C)C)cc1NC(=O)c1cnn2cc(-c3cnn(C)n3)sc12] | N-(5-((2-(2,2- dimethylpyrrolidin-1- yl)ethyl)carbamoyl)-2- methylpyridin-3-yl)-2-(2- methyl-2H-1,2,3-triazol-4- yl)pyrazolo[5,1-b]thiazole-7- carboxamide |
| 133 | 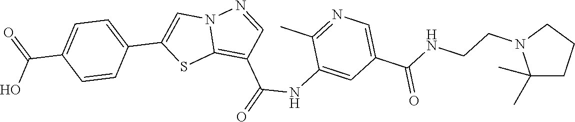 [SMILES: Cc1ncc(C(=O)NCCN2CCCC2(C)C)cc1NC(=O)c1cnn2cc(-c3ccc(C(=O)O)cc3)sc12] | 4-(7-((5-((2-(2,2- dimethylpyrrolidin-1- yl)ethyl)carbamoyl)-2- methylpyridin-3- yl)carbamoyl)pyrazolo[5,1- b]thiazol-2-yl)benzoic acid |
| 134 | 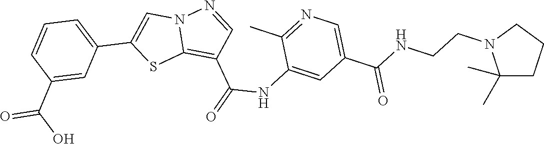 [SMILES: Cc1ncc(C(=O)NCCN2CCCC2(C)C)cc1NC(=O)c1cnn2cc(-c3cccc(C(=O)O)c3)sc12] | 3-(7-((5-((2-(2,2- dimethylpyrrolidin-1- yl)ethyl)carbamoyl)-2- methylpyridin-3- yl)carbamoyl)pyrazolo[5,1- b]thiazol-2-yl)benzoic acid |
| 135 | 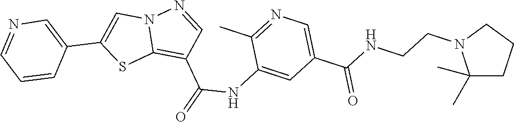 [SMILES: Cc1ncc(C(=O)NCCN2CCCC2(C)C)cc1NC(=O)c1cnn2cc(-c3cccnc3)sc12] | N-(5-((2-(2,2- dimethylpyrrolidin-1- yl)ethyl)carbamoyl)-2- methylpyridin-3-yl)-2-(pyridin- 3-yl)pyrazolo[5,1-b]thiazole-7- carboxamide |
| 136 | 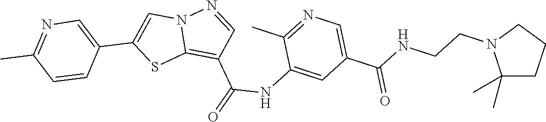 [SMILES: Cc1ccc(-c2cn3ncc(C(=O)Nc4cc(C(=O)NCCN5CCCC5(C)C)cnc4C)c3s2)cn1] | N-(5-((2-(2,2- dimethylpyrrolidin-1- yl)ethyl)carbamoyl)-2- methylpyridin-3-yl)-2-(6- methylpyridin-3- yl)pyrazolo[5,1-b]thiazole-7- carboxamide |
| 137 | 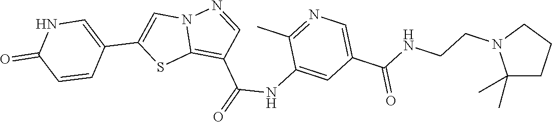 [SMILES: Cc1ncc(C(=O)NCCN2CCCC2(C)C)cc1NC(=O)c1cnn2cc(-c3ccc(=O)[nH]c3)sc12] | N-(5-((2-(2,2- dimethylpyrrolidin-1- yl)ethyl)carbamoyl)-2- methylpyridin-3-yl)-2-(6-oxo- 1,6-dihydropyridin-3- yl)pyrazolo[5,1-b]thiazole-7- carboxamide |
| 138 | 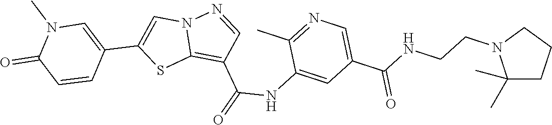 [SMILES: Cc1ncc(C(=O)NCCN2CCCC2(C)C)cc1NC(=O)c1cnn2cc(-c3ccc(=O)n(C)c3)sc12] | N-(5-((2-(2,2- dimethylpyrrolidin-1- yl)ethyl)carbamoyl)-2- methylpyridin-3-yl)-2-(1- methyl-6-oxo-1,6- dihydropyridin-3- yl)pyrazolo[5,1-b]thiazole-7- carboxamide |
| | 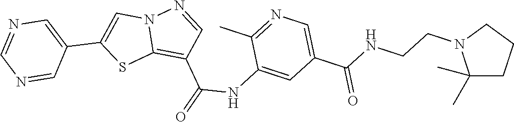 [SMILES: Cc1ncc(C(=O)NCCN2CCCC2(C)C)cc1NC(=O)c1cnn2cc(-c3cncnc3)sc12] | N-(5-((2-(2,2- dimethylpyrrolidin-1- yl)ethyl)carbamoyl)-2- methylpyridin-3-yl)-2- (pyrimidin-5-yl)pyrazolo[5,1- b]thiazole-7-carboxamide |
| | 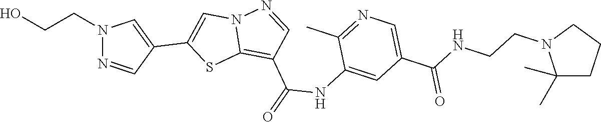 [SMILES: Cc1ncc(C(=O)NCCN2CCCC2(C)C)cc1NC(=O)c1cnn2cc(-c3cnn(CCO)c3)sc12] | N-(5-((2-(2,2- dimethylpyrrolidin-1- yl)ethyl)carbamoyl)-2- methylpyridin-3-yl)-2-(1-(2- hydroxyethyl)-1H-pyrazol-4- yl)pyrazolo[5,1-b]thiazole-7- carboxamide |
| 141 | 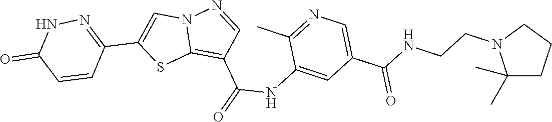 [SMILES: Cc1ncc(C(=O)NCCN2CCCC2(C)C)cc1NC(=O)c1cnn2cc(-c3ccc(=O)[nH]n3)sc12] | N-(5-((2-(2,2- dimethylpyrrolidin-1- yl)ethyl)carbamoyl)-2- methylpyridin-3-yl)-2-(6-oxo- 1,6-dihydropyridazin-3- yl)pyrazolo[5,1-b]thiazole-7- carboxamide |
| 142 | 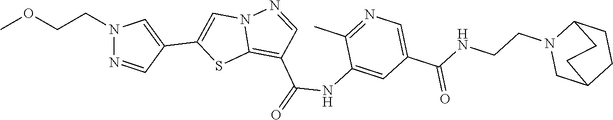 [SMILES: COCCn1cc(-c2cn3ncc(C(=O)Nc4cc(C(=O)NCCN5CC6CCC5CC6)cnc4C)c3s2)cn1] | N-(5-((2-(2- azabicyclo[2.2.2]octan-2- yl)ethyl)carbamoyl)-2- methylpyridin-3-yl)-2-(1-(2- methoxyethyl)-1H-pyrazol-4- yl)pyrazolo[5,1-b]thiazole-7- carboxamide |
| 143 | 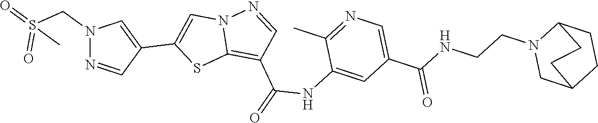 [SMILES: Cc1ncc(C(=O)NCCN2CC3CCC2CC3)cc1NC(=O)c1cnn2cc(-c3cnn(CS(C)(=O)=O)c3)sc12] | N-(5-((2-(2- azabicyclo[2.2.2]octan-2- yl)ethyl)carbamoyl)-2- methylpyridin-3-yl)-2-(1- ((methylsulfonyl)methyl)-1H- pyrazol-4-yl)pyrazolo[5,1- b]thiazole-7-carboxamide |
| 144 | 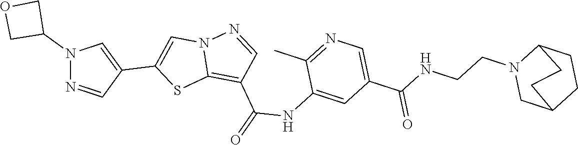 [SMILES: Cc1ncc(C(=O)NCCN2CC3CCC2CC3)cc1NC(=O)c1cnn2cc(-c3cnn(C4COC4)c3)sc12] | N-(5-((2-(2- azabicyclo[2.2.2]octan-2- yl)ethyl)carbamoyl)-2- methylpyridin-3-yl)-2-(1- (oxetan-3-yl)-1H-pyrazol-4- yl)pyrazolo[5,1-b]thiazole-7- carboxamide |
| 145 | 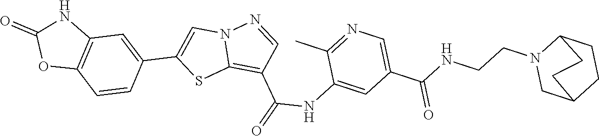 [SMILES: Cc1ncc(C(=O)NCCN2CC3CCC2CC3)cc1NC(=O)c1cnn2cc(-c3ccc4oc(=O)[nH]c4c3)sc12] | N-(5-((2-(2- azabicyclo[2.2.2]octan-2- yl)ethyl)carbamoyl)-2- methylpyridin-3-yl)-2-(2-oxo- 2,3-dihydrobenzo[d]oxazol-5- yl)pyrazolo[5,1-b]thiazole-7- carboxamide |
| 146 | 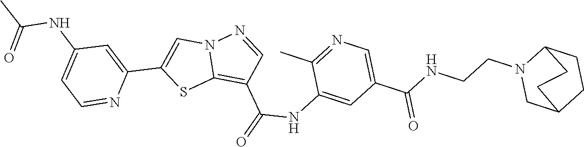 [SMILES: CC(=O)Nc1ccnc(-c2cn3ncc(C(=O)Nc4cc(C(=O)NCCN5CC6CCC5CC6)cnc4C)c3s2)c1] | N-(5-((2-(2- azabicyclo[2.2.2]octan-2- yl)ethyl)carbamoyl)-2- methylpyridin-3-yl)-2-(4- acetamidopyridin-2- yl)pyrazolo[5,1-b]thiazole-7- carboxamide |
| 147 | 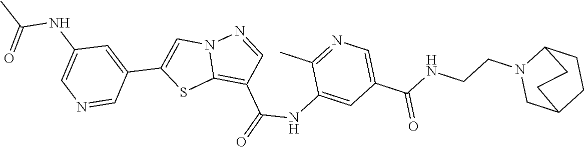 [SMILES: CC(=O)Nc1cncc(-c2cn3ncc(C(=O)Nc4cc(C(=O)NCCN5CC6CCC5CC6)cnc4C)c3s2)c1] | N-(5-((2-(2- azabicyclo[2.2.2]octan-2- yl)ethyl)carbamoyl)-2- methylpyridin-3-yl)-2-(5- acetamidopyridin-3- yl)pyrazolo[5,1-b]thiazole-7- carboxamide |
| 148 | 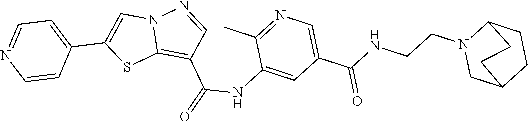 [SMILES: Cc1ncc(C(=O)NCCN2CC3CCC2CC3)cc1NC(=O)c1cnn2cc(-c3ccncc3)sc12] | N-(5-((2-(2- azabicyclo[2.2.2]octan-2- yl)ethyl)carbamoyl)-2- methylpyridin-3-yl)-2-(pyridin- 4-yl)pyrazolo[5,1-b]thiazole-7- carboxamide |
| 149 | 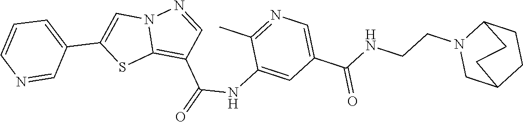 [SMILES: Cc1ncc(C(=O)NCCN2CC3CCC2CC3)cc1NC(=O)c1cnn2cc(-c3cccnc3)sc12] | N-(5-((2-(2- azabicyclo[2.2.2]octan-2- yl)ethyl)carbamoyl)-2- methylpyridin-3-yl)-2-(pyridin- 3-yl)pyrazolo[5,1-b]thiazole-7- carboxamide |
| 150 | 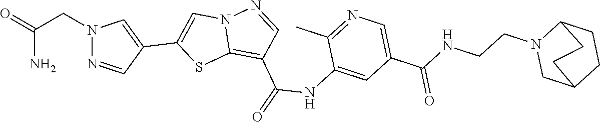 [SMILES: Cc1ncc(C(=O)NCCN2CC3CCC2CC3)cc1NC(=O)c1cnn2cc(-c3cnn(CC(N)=O)c3)sc12] | N-(5-((2-(2- azabicyclo[2.2.2]octan-2- yl)ethyl)carbamoyl)-2- methylpyridin-3-yl)-2-(1-(2- amino-2-oxoethyl)-1H-pyrazol- 4-yl)pyrazolo[5,1-b]thiazole-7- carboxamide |
| 151 | 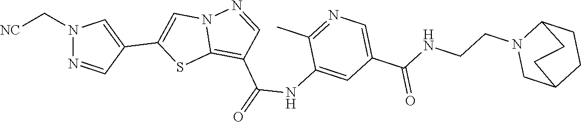 [SMILES: Cc1ncc(C(=O)NCCN2CC3CCC2CC3)cc1NC(=O)c1cnn2cc(-c3cnn(CC#N)c3)sc12] | N-(5-((2-(2- azabicyclo[2.2.2]octan-2- yl)ethyl)carbamoyl)-2- methylpyridin-3-yl)-2-(1- (cyanomethyl)-1H-pyrazol-4- yl)pyrazolo[5,1-b]thiazole-7- carboxamide |
| 152 | 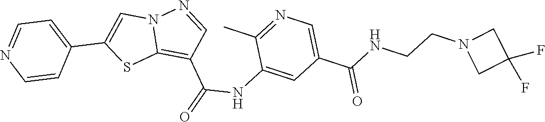 [SMILES: Cc1ncc(C(=O)NCCN2CC(F)(F)C2)cc1NC(=O)c1cnn2cc(-c3ccncc3)sc12] | N-(5-((2-(3,3-difluoroazetidin-1- yl)ethyl)carbamoyl)-2- methylpyridin-3-yl)-2-(pyridin- 4-yl)pyrazolo[5,1-b]thiazole-7- carboxamide |
| 153 | 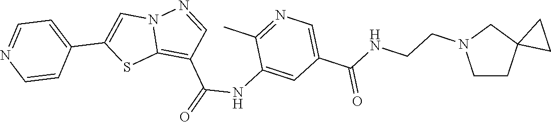 [SMILES: Cc1ncc(C(=O)NCCN2CCC3(CC3)C2)cc1NC(=O)c1cnn2cc(-c3ccncc3)sc12] | N-(5-((2-(5-azaspiro[2.4]heptan- 5-yl)ethyl)carbamoyl)-2- methylpyridin-3-yl)-2-(pyridin- 4-yl)pyrazolo[5,1-b]thiazole-7- carboxamide |
| 154 | 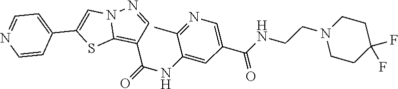 [SMILES: Cc1ncc(C(=O)NCCN2CCC(F)(F)CC2)cc1NC(=O)c1cnn2cc(-c3ccncc3)sc12] | N-(5-((2-(4,4-difluoropiperidin- 1-yl)ethyl)carbamoyl)-2- methylpyridin-3-yl)-2-(pyridin- 4-yl)pyrazolo[5,1-b]thiazole-7- carboxamide |
| 155 | 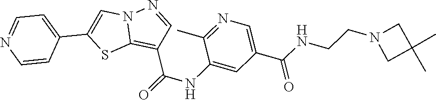 [SMILES: Cc1ncc(C(=O)NCCN2CC(C)(C)C2)cc1NC(=O)c1cnn2cc(-c3ccncc3)sc12] | N-(5-((2-(3,3-dimethylazetidin- 1-yl)ethyl)carbamoyl)-2- methylpyridin-3-yl)-2-(pyridin- 4-yl)pyrazolo[5,1-b]thiazole-7- carboxamide |
| 156 | 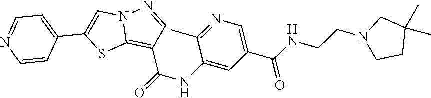 [SMILES: Cc1ncc(C(=O)NCCN2CCC(C)(C)C2)cc1NC(=O)c1cnn2cc(-c3ccncc3)sc12] | N-(5-((2-(3,3- dimethylpyrrolidin-1- yl)ethyl)carbamoyl)-2- methylpyridin-3-yl)-2-(pyridin- 4-yl)pyrazolo[5,1-b]thiazole-7- carboxamide |
| 157 | 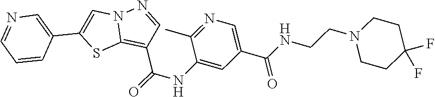 [SMILES: Cc1ncc(C(=O)NCCN2CCC(F)(F)CC2)cc1NC(=O)c1cnn2cc(-c3cccnc3)sc12] | N-(5-((2-(4,4-difluoropiperidin- 1-yl)ethyl)carbamoyl)-2- methylpyridin-3-yl)-2-(pyridin- 3-yl)pyrazolo[5,1-b]thiazole-7- carboxamide |
| 158 | 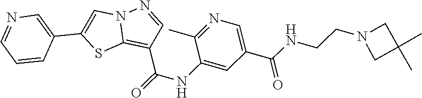 [SMILES: Cc1ncc(C(=O)NCCN2CC(C)(C)C2)cc1NC(=O)c1cnn2cc(-c3cccnc3)sc12] | N-(5-((2-(3,3-dimethylazetidin- 1-yl)ethyl)carbamoyl)-2- methylpyridin-3-yl)-2-(pyridin- 3-yl)pyrazolo[5,1-b]thiazole-7- carboxamide |
| 159 | 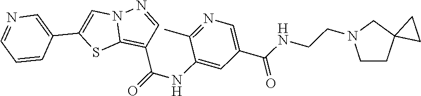 [SMILES: Cc1ncc(C(=O)NCCN2CCC3(CC3)C2)cc1NC(=O)c1cnn2cc(-c3cccnc3)sc12] | N-(5-((2-(5-azaspiro[2.4]heptan- 5-yl)ethyl)carbamoyl)-2- methylpyridin-3-yl)-2-(pyridin- 3-yl)pyrazolo[5,1-b]thiazole-7- carboxamide |
| 160 | 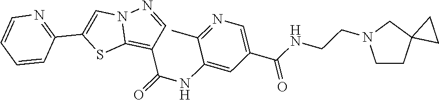 [SMILES: Cc1ncc(C(=O)NCCN2CCC3(CC3)C2)cc1NC(=O)c1cnn2cc(-c3ccccn3)sc12] | N-(5-((2-(5-azaspiro[2.4]heptan- 5-yl)ethyl)carbamoyl)-2- methylpyridin-3-yl)-2-(pyridin- 2-yl)pyrazolo[5,1-b]thiazole-7- carboxamide |
| 161 | 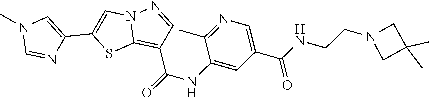 | N-(5-((2-(5-azaspiro[2.4]heptan- 5-yl)ethyl)carbamoyl)-2- methylpyridin-3-yl)-2-(1-(2- methoxyethyl)-1H-pyrazol-4- yl)pyrazolo[5,1-b]thiazole-7- carboxamide |
| 162 | 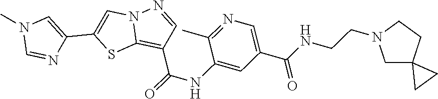 [SMILES: Cc1ncc(C(=O)NCCN2CCC3(CC3)C2)cc1NC(=O)c1cnn2cc(-c3cn(C)cn3)sc12] | N-(5-((2-(5-azaspiro[2.4]heptan- 5-yl)ethyl)carbamoyl)-2- methylpyridin-3-yl)-2-(1- methyl-1H-imidazol-4- yl)pyrazolo[5,1-b]thiazole-7- carboxamide |
| 163 | 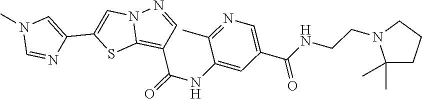 [SMILES: Cc1ncc(C(=O)NCCN2CCCC2(C)C)cc1NC(=O)c1cnn2cc(-c3cn(C)cn3)sc12] | N-(5-((2-(2,2- dimethylpyrrolidin-1- yl)ethyl)carbamoyl)-2- methylpyridin-3-yl)-2-(1- methyl-1H-imidazol-4- yl)pyrazolo[5,1-b]thiazole-7- carboxamide |
| 164 | 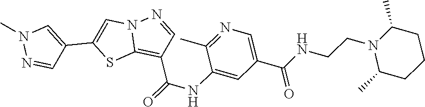 [SMILES: Cc1ncc(C(=O)NCCN2[C@H](C)CCC[C@@H]2C)cc1NC(=O)c1cnn2cc(-c3cnn(C)c3)sc12] | N-(2-(2,6-dimethylpiperidin-1- yl)ethyl)-6-methyl-5-((1-methyl- 6-((1-methyl-1H-pyrazol-4- yl)amino)-1H-pyrazolo[3,4- d]pyrimidin-3- yl)amino)nicotinamide |
| 165 | 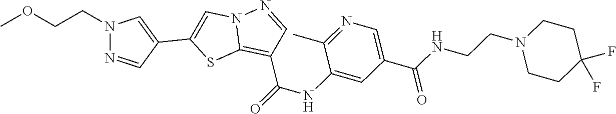 [SMILES: COCCn1cc(-c2cn3ncc(C(=O)Nc4cc(C(=O)NCCN5CCC(F)(F)CC5)cnc4C)c3s2)cn1] | N-(5-((2-(4,4-difluoropiperidin- 1-yl)ethyl)carbamoyl)-2- methylpyridin-3-yl)-2-(1-(2- methoxyethyl)-1H-pyrazol-4- yl)pyrazolo[5,1-b]thiazole-7- carboxamide |
| 166 | 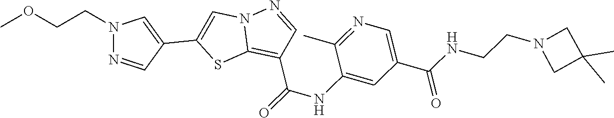 | N-(5-((2-(3,3-dimethylazetidin- 1-yl)ethyl)carbamoyl)-2- methylpyridin-3-yl)-2-(1-(2- methoxyethyl)-1H-pyrazol-4- yl)pyrazolo[5,1-b]thiazole-7- carboxamide |
| 167 | 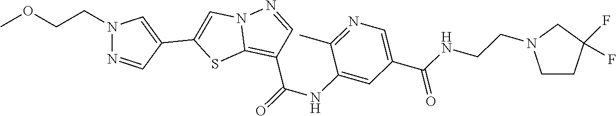 [SMILES: COCCn1cc(-c2cn3ncc(C(=O)Nc4cc(C(=O)NCCN5CCC(F)(F)C5)cnc4C)c3s2)cn1] | N-(5-((2-(3,3-difluoropyrrolidin- 1-yl)ethyl)carbamoyl)-2- methylpyridin-3-yl)-2-(1-(2- methoxyethyl)-1H-pyrazol-4- yl)pyrazolo[5,1-b]thiazole-7- carboxamide |
| 168 | 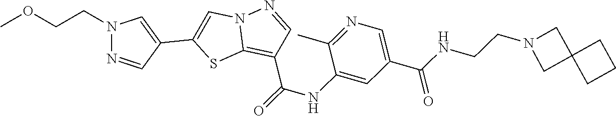 [SMILES: COCCn1cc(-c2cn3ncc(C(=O)Nc4cc(C(=O)NCCN5CC6(CCC6)C5)cnc4C)c3s2)cn1] | N-(5-((2-(2-azaspiro[3.3]heptan- 2-yl)ethyl)carbamoyl)-2- methylpyridin-3-yl)-2-(1-(2- methoxyethyl)-1H-pyrazol-4- yl)pyrazolo[5,1-b]thiazole-7- carboxamide |
| 169 | 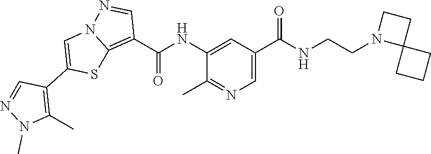 [SMILES: Cc1ncc(C(=O)NCCN2CCC23CCC3)cc1NC(=O)c1cnn2cc(-c3cnn(C)c3C)sc12] | N-(5-((2-(1-azaspiro[3.3]heptan- 1-yl)ethyl)carbamoyl)-2- methylpyridin-3-yl)-2-(1,5- dimethyl-1H-pyrazol-4- yl)pyrazolo[5,1-b]thiazole-7- carboxamide |
| 170 | 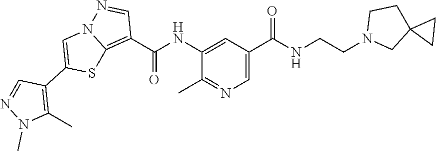 [SMILES: Cc1ncc(C(=O)NCCN2CCC3(CC3)C2)cc1NC(=O)c1cnn2cc(-c3cnn(C)c3C)sc12] | N-(5-((2-(5-azaspiro[2.4]heptan- 5-yl)ethyl)carbamoyl)-2- methylpyridin-3-yl)-2-(1,5- dimethyl-1H-pyrazol-4- yl)pyrazolo[5,1-b]thiazole-7- carboxamide |
| 171 | 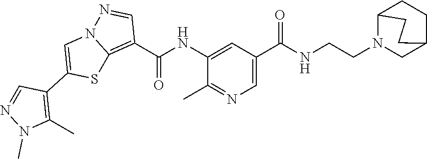 [SMILES: Cc1ncc(C(=O)NCCN2CC3CCC2CC3)cc1NC(=O)c1cnn2cc(-c3cnn(C)c3C)sc12] | N-(5-((2-(2- azabicyclo[2.2.2]octan-2- yl)ethyl)carbamoyl)-2- methylpyridin-3-yl)-2-(1,5- dimethyl-1H-pyrazol-4- yl)pyrazolo[5,1-b]thiazole-7- carboxamide |
| 172 | 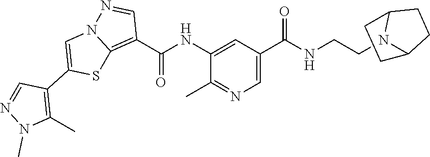 [SMILES: Cc1ncc(C(=O)NCCN2C3CCC2CC3)cc1NC(=O)c1cnn2cc(-c3cnn(C)c3C)sc12] | N-(5-((2-(7- azabicyclo[2.2.1]heptan-7- yl)ethyl)carbamoyl)-2- methylpyridin-3-yl)-2-(1,5- dimethyl-1H-pyrazol-4- yl)pyrazolo[5,1-b]thiazole-7- carboxamide |
| 173 | 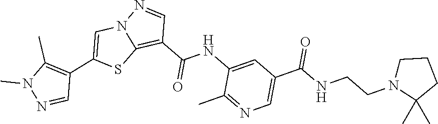 [SMILES: Cc1ncc(C(=O)NCCN2CCCC2(C)C)cc1NC(=O)c1cnn2cc(-c3cnn(C)c3C)sc12] | 2-(1,5-dimethyl-1H-pyrazol-4- yl)-N-(5-((2-(2,2- dimethylpyrrolidin-1- yl)ethyl)carbamoyl)-2- methylpyridin-3- yl)pyrazolo[5,1-b]thiazole-7- carboxamide |
| 174 | 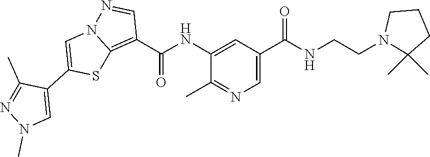 [SMILES: Cc1ncc(C(=O)NCCN2CCCC2(C)C)cc1NC(=O)c1cnn2cc(-c3cn(C)nc3C)sc12] | 2-(1,3-dimethyl-1H-pyrazol-4- yl)-N-(5-((2-(2,2- dimethylpyrrolidin-1- yl)ethyl)carbamoyl)-2- methylpyridin-3- yl)pyrazolo[5,1-b]thiazole-7- carboxamide |
| 175 | 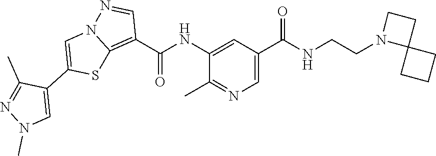 [SMILES: Cc1ncc(C(=O)NCCN2CCC23CCC3)cc1NC(=O)c1cnn2cc(-c3cn(C)nc3C)sc12] | N-(5-((2-(1-azaspiro[3.3]heptan- 1-yl)ethyl)carbamoyl)-2- methylpyridin-3-yl)-2-(1,3- dimethyl-1H-pyrazol-4- yl)pyrazolo[5,1-b]thiazole-7- carboxamide |
| 176 | 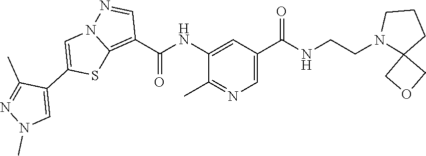 [SMILES: Cc1ncc(C(=O)NCCN2CCCC23COC3)cc1NC(=O)c1cnn2cc(-c3cn(C)nc3C)sc12] | N-(5-((2-(2-oxa-5- azaspiro[3.4]octan-5- yl)ethyl)carbamoyl)-2- methylpyridin-3-yl)-2-(1,3- dimethyl-1H-pyrazol-4- yl)pyrazolo[5,1-b]thiazole-7- carboxamide |
| 177 | 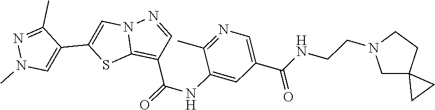 [SMILES: Cc1ncc(C(=O)NCCN2CCC3(CC3)C2)cc1NC(=O)c1cnn2cc(-c3cn(C)nc3C)sc12] | N-(5-((2-(5-azaspiro[2.4]heptan- 5-yl)ethyl)carbamoyl)-2- methylpyridin-3-yl)-2-(1,3- dimethyl-1H-pyrazol-4- yl)pyrazolo[5,1-b]thiazole-7- carboxamide |
| 178 | 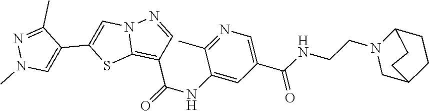 [SMILES: Cc1ncc(C(=O)NCCN2CC3CCC2CC3)cc1NC(=O)c1cnn2cc(-c3cn(C)nc3C)sc12] | N-(5-((2-(2- azabicyclo[2.2.2]octan-2- yl)ethyl)carbamoyl)-2- methylpyridin-3-yl)-2-(1,3- dimethyl-1H-pyrazol-4- yl)pyrazolo[5,1-b]thiazole-7- carboxamide |
| 179 | 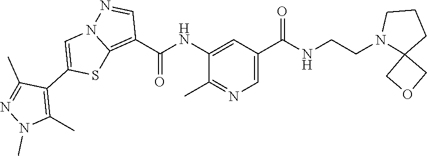 [SMILES: Cc1ncc(C(=O)NCCN2CCCC23COC3)cc1NC(=O)c1cnn2cc(-c3c(C)nn(C)c3C)sc12] | N-(5-((2-(2-oxa-5- azaspiro[3.4]octan-5- yl)ethyl)carbamoyl)-2- methylpyridin-3-yl)-2-(1,3,5- trimethyl-1H-pyrazol-4- yl)pyrazolo[5,1-b]thiazole-7- carboxamide |
| 180 | 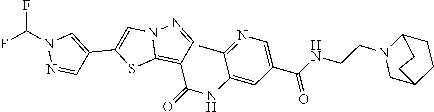 [SMILES: Cc1ncc(C(=O)NCCN2CC3CCC2CC3)cc1NC(=O)c1cnn2cc(-c3cnn(C(F)F)c3)sc12] | N-(5-((2-(2- azabicyclo[2.2.2]octan-2- yl)ethyl)carbamoyl)-2- methylpyridin-3-yl)-2-(1- (difluoromethyl)-1H-pyrazol-4- yl)pyrazolo[5,1-b]thiazole-7- carboxamide |
| 181 | 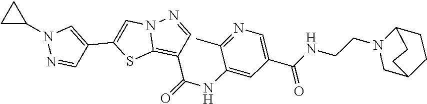 [SMILES: Cc1ncc(C(=O)NCCN2CC3CCC2CC3)cc1NC(=O)c1cnn2cc(-c3cnn(C4CC4)c3)sc12] | N-(5-((2-(2- azabicyclo[2.2.2]octan-2- yl)ethyl)carbamoyl)-2- methylpyridin-3-yl)-2-(1- cyclopropyl-1H-pyrazol-4- yl)pyrazolo[5,1-b]thiazole-7- carboxamide |
| 182 | 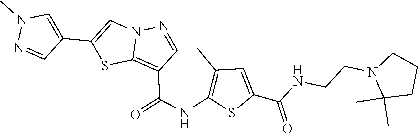 [SMILES: Cc1cc(C(=O)NCCN2CCCC2(C)C)sc1NC(=O)c1cnn2cc(-c3cnn(C)c3)sc12] | N-(5-((2-(2,2- dimethylpyrrolidin-1- yl)ethyl)carbamoyl)-3- methylthiophen-2-yl)-2-(1- methyl-1H-pyrazol-4- yl)pyrazolo[5,1-b]thiazole-7- carboxamide |
| 183 | 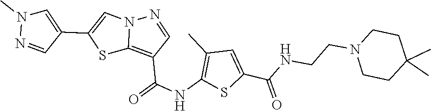 | N-(5-((2-(4,4-dimethylpiperidin- 1-yl)ethyl)carbamoyl)-3- methylthiophen-2-yl)-2-(1- methyl-1H-pyrazol-4- yl)pyrazolo[5,1-b]thiazole-7- carboxamide |
| 184 | 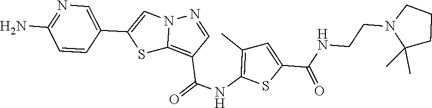 [SMILES: Cc1cc(C(=O)NCCN2CCCC2(C)C)sc1NC(=O)c1cnn2cc(-c3ccc(N)nc3)sc12] | 2-(6-aminopyridin-3-yl)-N-(5- ((2-(2,2-dimethylpyrrolidin-1- yl)ethyl)carbamoyl)-3- methylthiophen-2- yl)pyrazolo[5,1-b]thiazole-7- carboxamide |
| 185 | 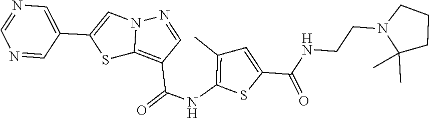 [SMILES: Cc1cc(C(=O)NCCN2CCCC2(C)C)sc1NC(=O)c1cnn2cc(-c3cncnc3)sc12] | N-(5-((2-(2,2- dimethylpyrrolidin-1- yl)ethyl)carbamoyl)-3- methylthiophen-2-yl)-2- (pyrimidin-5-yl)pyrazolo[5,1- b]thiazole-7-carboxamide |
| 186 | 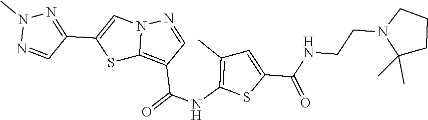 [SMILES: Cc1cc(C(=O)NCCN2CCCC2(C)C)sc1NC(=O)c1cnn2cc(-c3cnn(C)n3)sc12] | N-(5-((2-(2,2- dimethylpyrrolidin-1- yl)ethyl)carbamoyl)-3- methylthiophen-2-yl)-2-(2- methyl-2H-1,2,3-triazol-4- yl)pyrazolo[5,1-b]thiazole-7- carboxamide |
| 187 | 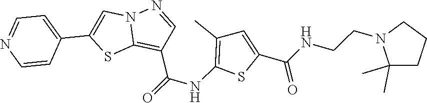 [SMILES: Cc1cc(C(=O)NCCN2CCCC2(C)C)sc1NC(=O)c1cnn2cc(-c3ccncc3)sc12] | N-(5-((2-(2,2- dimethylpyrrolidin-1- yl)ethyl)carbamoyl)-3- methylthiophen-2-yl)-2-(pyridin- 4-yl)pyrazolo[5,1-b]thiazole-7- carboxamide |
| 188 | 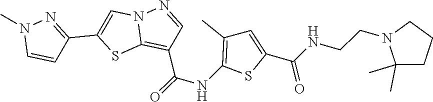 [SMILES: Cc1cc(C(=O)NCCN2CCCC2(C)C)sc1NC(=O)c1cnn2cc(-c3ccn(C)n3)sc12] | N-(5-((2-(2,2- dimethylpyrrolidin-1- yl)ethyl)carbamoyl)-3- methylthiophen-2-yl)-2-(1- methyl-1H-pyrazol-3- yl)pyrazolo[5,1-b]thiazole-7- carboxamide |
| 189 | 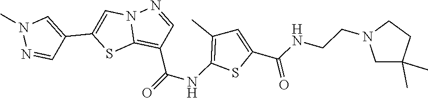 | N-(5-((2-(3,3- dimethylpyrrolidin-1- yl)ethyl)carbamoyl)-3- methylthiophen-2-yl)-2-(1- methyl-1H-pyrazol-4- yl)pyrazolo[5,1-b]thiazole-7- carboxamide |
| 190 | 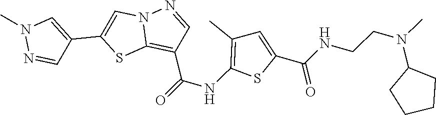 [SMILES: Cc1cc(C(=O)NCCN(C)C2CCCC2)sc1NC(=O)c1cnn2cc(-c3cnn(C)c3)sc12] | N-(5-((2- (cyclopentyl(methyl)amino)ethyl) carbamoyl)-3-methylthiophen- 2-yl)-2-(1-methyl-1H-pyrazol-4- yl)pyrazolo[5,1-b]thiazole-7- carboxamide |
| 191 | 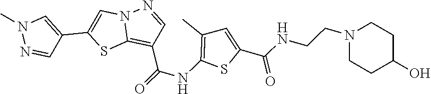 [SMILES: Cc1cc(C(=O)NCCN2CCC(O)CC2)sc1NC(=O)c1cnn2cc(-c3cnn(C)c3)sc12] | N-(5-((2-(4-hydroxypiperidin-1- yl)ethyl)carbamoyl)-3- methylthiophen-2-yl)-2-(1- methyl-1H-pyrazol-4- yl)pyrazolo[5,1-b]thiazole-7- carboxamide |
| 192 | 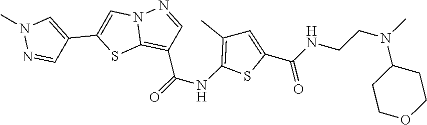 [SMILES: Cc1cc(C(=O)NCCN(C)C2CCOCC2)sc1NC(=O)c1cnn2cc(-c3cnn(C)c3)sc12] | 2-(1-methyl-1H-pyrazol-4-yl)- N-(3-methyl-5-((2- (methyl(tetrahydro-2H-pyran-4- yl)amino)ethyl)carbamoyl)thiophen- 2-yl)pyrazolo[5,1- b]thiazole-7-carboxamide |
| 193 | 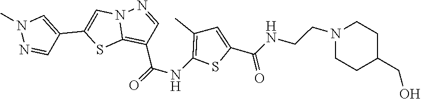 [SMILES: Cc1cc(C(=O)NCCN2CCC(CO)CC2)sc1NC(=O)c1cnn2cc(-c3cnn(C)c3)sc12] | N-(5-((2-(4- (hydroxymethyl)piperidin-1- yl)ethyl)carbamoyl)-3- methylthiophen-2-yl)-2-(1- methyl-1H-pyrazol-4- yl)pyrazolo[5,1-b]thiazole-7- carboxamide |
| 194 | 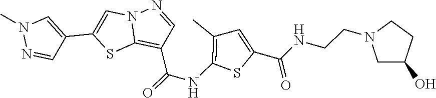 [SMILES: Cc1cc(C(=O)NCCN2CC[C@@H](O)C2)sc1NC(=O)c1cnn2cc(-c3cnn(C)c3)sc12] | (R)-N-(5-((2-(3- hydroxypyrrolidin-1- yl)ethyl)carbamoyl)-3- methylthiophen-2-yl)-2-(1- methyl-1H-pyrazol-4- yl)pyrazolo[5,1-b]thiazole-7- carboxamide |
| 195 | 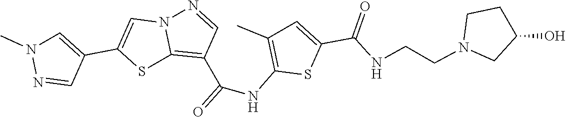 [SMILES: Cc1cc(C(=O)NCCN2CC[C@H](O)C2)sc1NC(=O)c1cnn2cc(-c3cnn(C)c3)sc12] | (S)-N-(5-((2-(3- hydroxypyrrolidin-1- yl)ethyl)carbamoyl)-3- methylthiophen-2-yl)-2-(1- methyl-1H-pyrazol-4- yl)pyrazolo[5,1-b]thiazole-7- carboxamide |
| 196 | 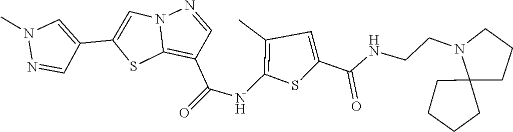 [SMILES: Cc1cc(C(=O)NCCN2CCCC23CCCC3)sc1NC(=O)c1cnn2cc(-c3cnn(C)c3)sc12] | N-(5-((2-(1-azaspiro[4.4]nonan- 1-yl)ethyl)carbamoyl)-3- methylthiophen-2-yl)-2-(1- methyl-1H-pyrazol-4- yl)pyrazolo[5,1-b]thiazole-7- carboxamide |
| 197 | 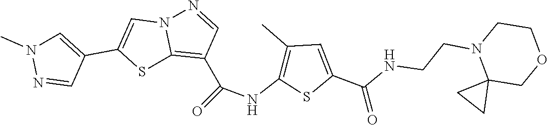 [SMILES: Cc1cc(C(=O)NCCN2CCOCC23CC3)sc1NC(=O)c1cnn2cc(-c3cnn(C)c3)sc12] | N-(5-((2-(7-oxa-4- azaspiro[2.5]octan-4- yl)ethyl)carbamoyl)-3- methylthiophen-2-yl)-2-(1- methyl-1H-pyrazol-4- yl)pyrazolo[5,1-b]thiazole-7- carboxamide |
| 198 | 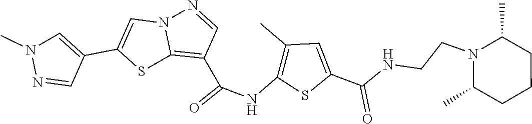 [SMILES: Cc1cc(C(=O)NCCN2[C@H](C)CCC[C@@H]2C)sc1NC(=O)c1cnn2cc(-c3cnn(C)c3)sc12] | N-(5-((2-((2S,6R)-2,6- dimethylpiperidin-1- yl)ethyl)carbamoyl)-3- methylthiophen-2-yl)-2-(1- methyl-1H-pyrazol-4- yl)pyrazolo[5,1-b]thiazole-7- carboxamide |
| 199 | 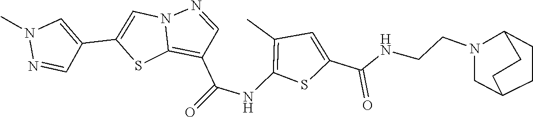 [SMILES: Cc1cc(C(=O)NCCN2CC3CCC2CC3)sc1NC(=O)c1cnn2cc(-c3cnn(C)c3)sc12] | N-(5-((2-(2- azabicyclo[2.2.2]octan-2- yl)ethyl)carbamoyl)-3- methylthiophen-2-yl)-2-(1- methyl-1H-pyrazol-4- yl)pyrazolo[5,1-b]thiazole-7- carboxamide |
| 200 | 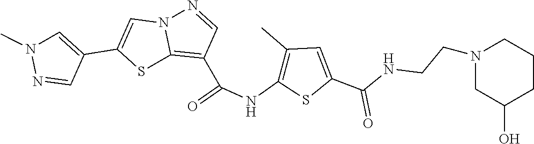 [SMILES: Cc1cc(C(=O)NCCN2CCCC(O)C2)sc1NC(=O)c1cnn2cc(-c3cnn(C)c3)sc12] | N-(5-((2-(3-hydroxypiperidin-1- yl)ethyl)carbamoyl)-3- methylthiophen-2-yl)-2-(1- methyl-1H-pyrazol-4- yl)pyrazolo[5,1-b]thiazole-7- carboxamide |
| 201 | 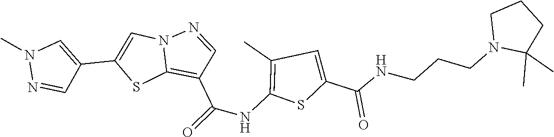 | N-(5-((3-(2,2- dimethylpyrrolidin-1- yl)propyl)carbamoyl)-3- methylthiophen-2-yl)-2-(1- methyl-1H-pyrazol-4- yl)pyrazolo[5,1-b]thiazole-7- carboxamide |
| 202 | 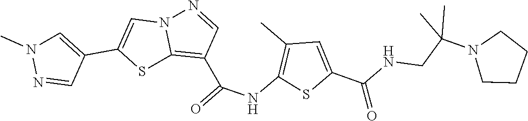 [SMILES: Cc1cc(C(=O)NCC(C)(C)N2CCCC2)sc1NC(=O)c1cnn2cc(-c3cnn(C)c3)sc12] | 2-(1-methyl-1H-pyrazol-4-yl)- N-(3-methyl-5-((2-methyl-2- (pyrrolidin-1- yl)propyl)carbamoyl)thiophen-2- yl)pyrazolo[5,1-b]thiazole-7- carboxamide |
| 203 | 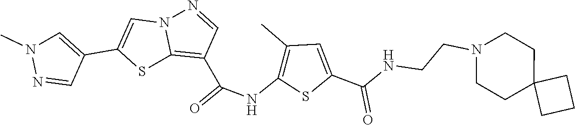 [SMILES: Cc1cc(C(=O)NCCN2CCC3(CCC3)CC2)sc1NC(=O)c1cnn2cc(-c3cnn(C)c3)sc12] | N-(5-((2-(7-azaspiro[3.5]nonan- 7-yl)ethyl)carbamoyl)-3- methylthiophen-2-yl)-2-(1- methyl-1H-pyrazol-4- yl)pyrazolo[5,1-b]thiazole-7- carboxamide |
| 204 | 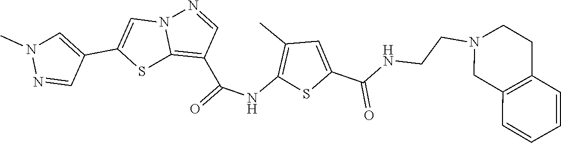 [SMILES: Cc1cc(C(=O)NCCN2CCc3ccccc3C2)sc1NC(=O)c1cnn2cc(-c3cnn(C)c3)sc12] | N-(5-((2-(3,4- dihydroisoquinolin-2(1H)- yl)ethyl)carbamoyl)-3- methylthiophen-2-yl)-2-(1- methyl-1H-pyrazol-4- yl)pyrazolo[5,1-b]thiazole-7- carboxamide |
| 205 | 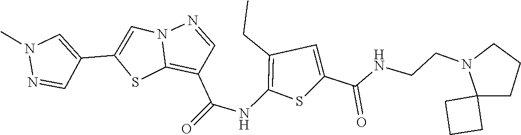 [SMILES: CCc1cc(C(=O)NCCN2CCCC23CCC3)sc1NC(=O)c1cnn2cc(-c3cnn(C)c3)sc12] | N-(5-((2-(5-azaspiro[3.4]octan- 5-yl)ethyl)carbamoyl)-3- ethylthiophen-2-yl)-2-(1-methyl- 1H-pyrazol-4-yl)pyrazolo[5,1- b]thiazole-7-carboxamide |
| 206 | 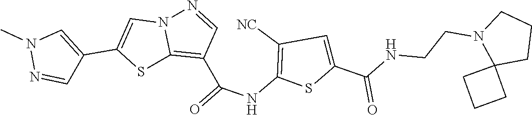 [SMILES: Cn1cc(-c2cn3ncc(C(=O)Nc4sc(C(=O)NCCN5CCCC56CCC6)cc4C#N)c3s2)cn1] | N-(5-((2-(5-azaspiro[3.4]octan- 5-yl)ethyl)carbamoyl)-3- cyanothiophen-2-yl)-2-(1- methyl-1H-pyrazol-4- yl)pyrazolo[5,1-b]thiazole-7- carboxamide |
| 207 | 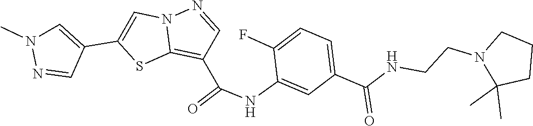 [SMILES: Cn1cc(-c2cn3ncc(C(=O)Nc4cc(C(=O)NCCN5CCCC5(C)C)ccc4F)c3s2)cn1] | N-(5-((2-(2,2- dimethylpyrrolidin-1- yl)ethyl)carbamoyl)-2- fluorophenyl)-2-(1-methyl-1H- pyrazol-4-yl)pyrazolo[5,1- b]thiazole-7-carboxamide |
| 208 | 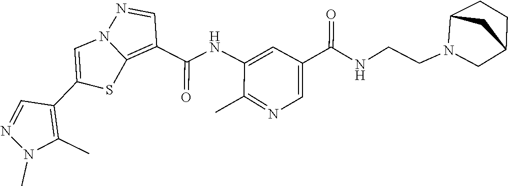 [SMILES: Cc1ncc(C(=O)NCCN2C[C@H]3CC[C@@H]2C3)cc1NC(=O)c1cnn2cc(-c3cnn(C)c3C)sc12] | N-(5-((2-((1R,4S)-2- azabicyclo[2.2.1]heptan-2- yl)ethyl)carbamoyl)-2- methylpyridin-3-yl)-2-(1,5- dimethyl-1H-pyrazol-4- yl)pyrazolo[5,1-b]thiazole-7- carboxamide |
| 209 | 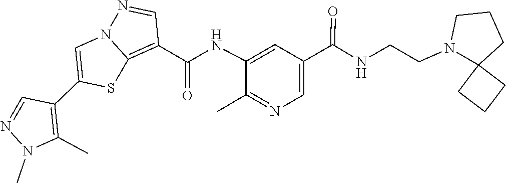 [SMILES: Cc1ncc(C(=O)NCCN2CCCC23CCC3)cc1NC(=O)c1cnn2cc(-c3cnn(C)c3C)sc12] | N-(5-((2-(5-azaspiro[3.4]octan- 5-yl)ethyl)carbamoyl)-2-methyl- pyridin-3-yl)-2-(1,5-dimethyl- 1H-pyrazol-4-yl)pyrazolo[5,1- b]thiazole-7-carboxamide |
| 210 | 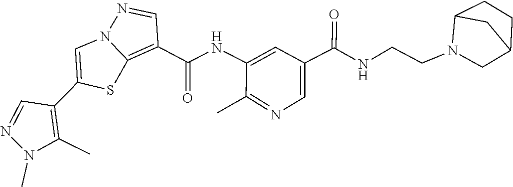 | N-(5-((2-(2- azabicyclo[2.2.1]heptan-2- yl)ethyl)carbamoyl)-2- methylpyridin-3-yl)-2-(1,5- dimethyl-1H-pyrazol-4- yl)pyrazolo[5,1-b]thiazole-7- carboxamide |
| 211 | 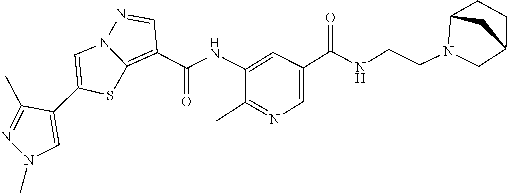 [SMILES: Cc1ncc(C(=O)NCCN2C[C@H]3CC[C@@H]2C3)cc1NC(=O)c1cnn2cc(-c3cn(C)nc3C)sc12] | N-(5-((2-((1R,4S)-2- azabicyclo[2.2.1]heptan-2- yl)ethyl)carbamoyl)-2- methylpyridin-3-yl)-2-(1,3- dimethyl-1H-pyrazol-4- yl)pyrazolo[5,1-b]thiazole-7- carboxamide |
| 212 | 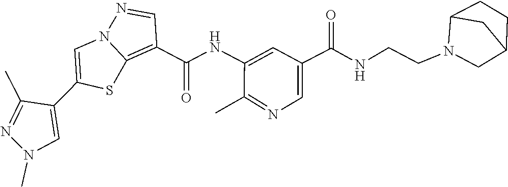 [SMILES: Cc1ncc(C(=O)NCCN2CC3CCC2C3)cc1NC(=O)c1cnn2cc(-c3cn(C)nc3C)sc12] | N-(5-((2-(2- azabicyclo[2.2.1]heptan-2- yl)ethyl)carbamoyl)-2- methylpyridin-3-yl)-2-(1,3- dimethyl-1H-pyrazol-4- yl)pyrazolo[5,1-b]thiazole-7- carboxamide |
| 213 | 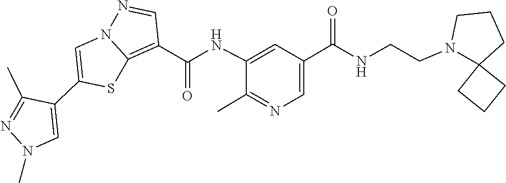 [SMILES: Cc1ncc(C(=O)NCCN2CCCC23CCC3)cc1NC(=O)c1cnn2cc(-c3cn(C)nc3C)sc12] | N-(5-((2-(5-azaspiro[3.4]octan- 5-yl)ethyl)carbamoyl)-2- methylpyridin-3-yl)-2-(1,3- dimethyl-1H-pyrazol-4- yl)pyrazolo[5,1-b]thiazole-7- carboxamide |
| 214 | 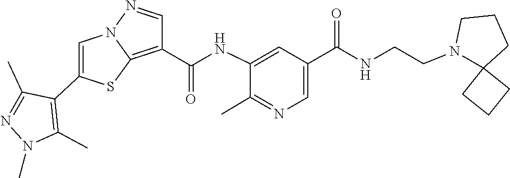 [SMILES: Cc1ncc(C(=O)NCCN2CCCC23CCC3)cc1NC(=O)c1cnn2cc(-c3c(C)nn(C)c3C)sc12] | N-(5-((2-(5-azaspiro[3.4]octan- 5-yl)ethyl)carbamoyl)-2- methylpyridin-3-yl)-2-(1,3,5- trimethyl-1H-pyrazol-4- yl)pyrazolo[5,1-b]thiazole-7- carboxamide |
| 215 | 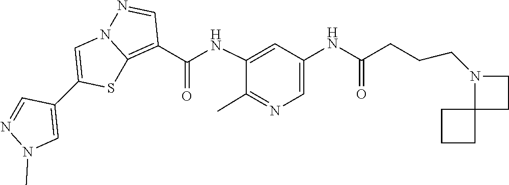 [SMILES: Cc1ncc(NC(=O)CCCN2CCC23CCC3)cc1NC(=O)c1cnn2cc(-c3cnn(C)c3)sc12] | N-(5-(4-(1-azaspiro[3.3]heptan- 1-yl)butanamido)-2- methylpyridin-3-yl)-2-(1- methyl-1H-pyrazol-4- yl)pyrazolo[5,1-b]thiazole-7- carboxamide |
| 216 | 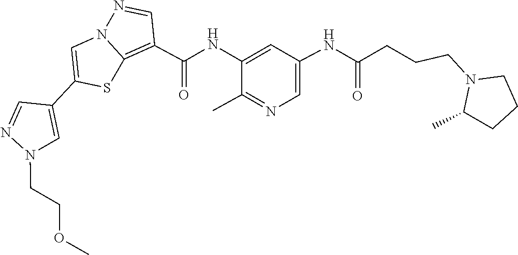 [SMILES: COCCn1cc(-c2cn3ncc(C(=O)Nc4cc(NC(=O)CCCN5CCC[C@@H]5C)cnc4C)c3s2)cn1] | (S)-2-(1-(2-methoxyethyl)-1H- pyrazol-4-yl)-N-(2-methyl-5-(4- (2-methylpyrrolidin-1- yl)butanamido)pyridin-3- yl)pyrazolo[5,1-b]thiazole-7- carboxamide |
| 217 | 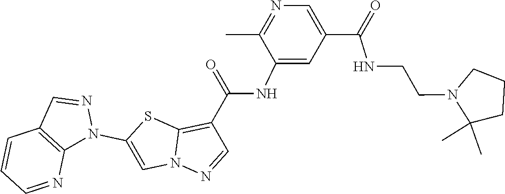 [SMILES: Cc1ncc(C(=O)NCCN2CCCC2(C)C)cc1NC(=O)c1cnn2cc(-n3ncc4cccnc43)sc12] | N-(5-((2-(2,2- dimethylpyrrolidin-1- yl)ethyl)carbamoyl)-2- methylpyridin-3-yl)-2-(1H- pyrazolo[3,4-b]pyridin-1- yl)pyrazolo[5,1-b]thiazole-7- carboxamide |
| 218 | 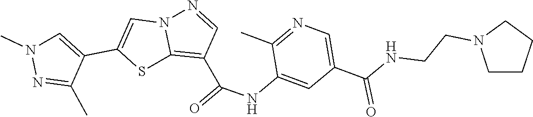 [SMILES: Cc1ncc(C(=O)NCCN2CCCC2)cc1NC(=O)c1cnn2cc(-c3cn(C)nc3C)sc12] | 2-(1,3-Dimethyl-1H-pyrazol-4- yl)-N-(2-methyl-5-((2- (pyrrolidin-1- yl)ethyl)carbamoyl)pyridin-3- yl)pyrazolo[5,1-b]thiazole-7- carboxamide |
| 219 | 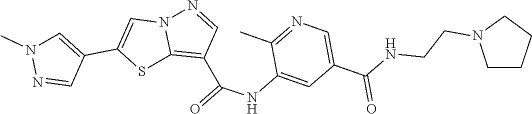 [SMILES: Cc1ncc(C(=O)NCCN2CCCC2)cc1NC(=O)c1cnn2cc(-c3cnn(C)c3)sc12] | 2-(1-Methyl-1H-pyrazol-4-yl)- N-(2-methyl-5-((2-(pyrrolidin-1- yl)ethyl)carbamoyl)pyridin-3- yl)pyrazolo[5,1-b]thiazole-7- carboxamide |
| 220 | 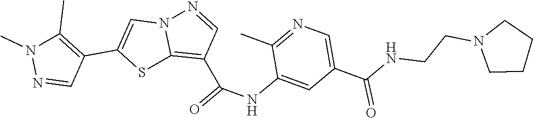 [SMILES: Cc1ncc(C(=O)NCCN2CCCC2)cc1NC(=O)c1cnn2cc(-c3cnn(C)c3C)sc12] | 2-(1,5-Dimethyl-1H-pyrazol-4- yl)-N-(2-methyl-5-((2- (pyrrolidin-1- yl)ethyl)carbamoyl)pyridin-3- yl)pyrazolo[5,1-b]thiazole-7- carboxamide |
| 221 | 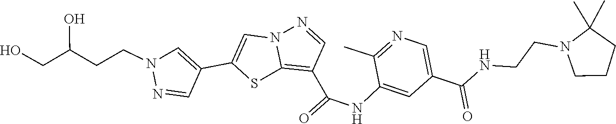 [SMILES: Cc1ncc(C(=O)NCCN2CCCC2(C)C)cc1NC(=O)c1cnn2cc(-c3cnn(CCC(O)CO)c3)sc12] | 2-(1-(3,4-Dihydroxybutyl)-1H- pyrazol-4-yl)-N-(5-((2-(2,2- dimethylpyrrolidin-1- yl)ethyl)carbamoyl)-2- methylpyridin-3- yl)pyrazolo[5,1-b]thiazole-7- carboxamide |
| 222 | 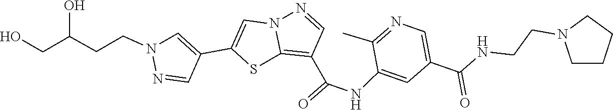 [SMILES: Cc1ncc(C(=O)NCCN2CCCC2)cc1NC(=O)c1cnn2cc(-c3cnn(CCC(O)CO)c3)sc12] | 2-(1-(3,4-Dihydroxybutyl)-1H- pyrazol-4-yl)-N-(2-methyl-5-((2- (pyrrolidin-1- yl)ethyl)carbamoyl)pyridin-3- yl)pyrazolo[5,1-b]thiazole-7- carboxamide |
| 223 | 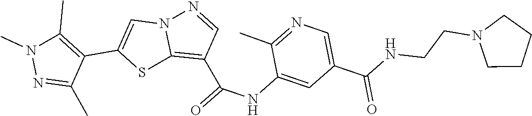 [SMILES: Cc1ncc(C(=O)NCCN2CCCC2)cc1NC(=O)c1cnn2cc(-c3c(C)nn(C)c3C)sc12] | N-(2-methyl-5-((2-(pyrrolidin-1- yl)ethyl)carbamoyl)pyridin-3- yl)-2-(1,3,5-trimethyl-1H- pyrazol-4-yl)pyrazolo[5,1- b]thiazole-7-carboxamide |
| 224 | 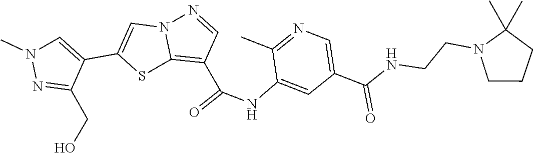 [SMILES: Cc1ncc(C(=O)NCCN2CCCC2(C)C)cc1NC(=O)c1cnn2cc(-c3cn(C)nc3CO)sc12] | N-(5-((2-(2,2- dimethylpyrrolidin-1- yl)ethyl)carbamoyl)-2- methylpyridin-3-yl)-2-(3- (hydroxymethyl)-1-methyl-1H- pyrazol-4-yl)pyrazolo[5,1- b]thiazole-7-carboxamide |
| 225 | 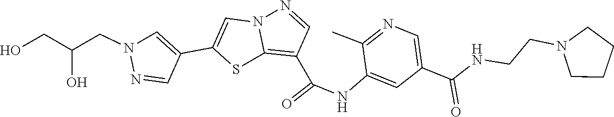 [SMILES: Cc1ncc(C(=O)NCCN2CCCC2)cc1NC(=O)c1cnn2cc(-c3cnn(CC(O)CO)c3)sc12] | 2-(1-(2,3-Dihydroxypropyl)-1H- pyrazol-4-yl)-N-(2-methyl-5-((2- (pyrrolidin-1- yl)ethyl)carbamoyl)pyridin-3- yl)pyrazolo[5,1-b]thiazole-7- carboxamide |
| 226 | 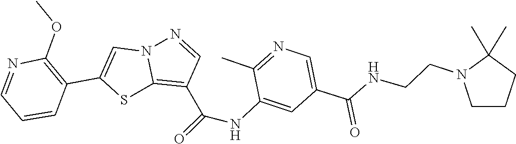 [SMILES: COc1ncccc1-c1cn2ncc(C(=O)Nc3cc(C(=O)NCCN4CCCC4(C)C)cnc3C)c2s1] | N-(5-((2-(2,2- Dimethylpyrrolidin-1- yl)ethyl)carbamoyl)-2- methylpyridin-3-yl)-2-(2- methoxypyridin-3- yl)pyrazolo[5,1-b]thiazole-7- carboxamide |
| 227 | 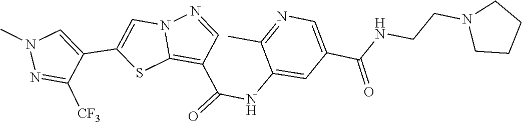 [SMILES: Cc1ncc(C(=O)NCCN2CCCC2)cc1NC(=O)c1cnn2cc(-c3cn(C)nc3C(F)(F)F)sc12] | 2-(1-Methyl-3-(trifluoromethyl)- 1H-pyrazol-4-yl)-N-(2-methyl- 5-((2-(pyrrolidin-1- yl)ethyl)carbamoyl)pyridin-3- yl)pyrazolo[5,1-b]thiazole-7- carboxamide |
| 228 | 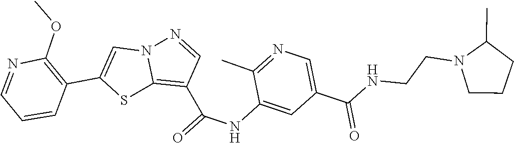 [SMILES: COc1ncccc1-c1cn2ncc(C(=O)Nc3cc(C(=O)NCCN4CCCC4C)cnc3C)c2s1] | 2-(2-Methoxypyridin-3-yl)-N- (2-methyl-5-((2-(2- methylpyrrolidin-1- yl)ethyl)carbamoyl)pyridin-3- yl)pyrazolo[5,1-b]thiazole-7- carboxamide |
| 229 | 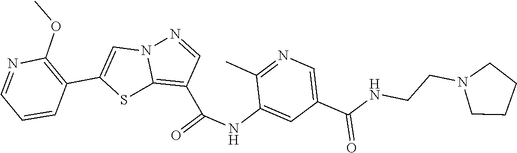 [SMILES: COc1ncccc1-c1cn2ncc(C(=O)Nc3cc(C(=O)NCCN4CCCC4)cnc3C)c2s1] | 2-(2-Methoxypyridin-3-yl)-N- (2-methyl-5-((2-(pyrrolidin-1- yl)ethyl)carbamoyl)pyridin-3- yl)pyrazolo[5,1-b]thiazole-7- carboxamide |
| 230 | 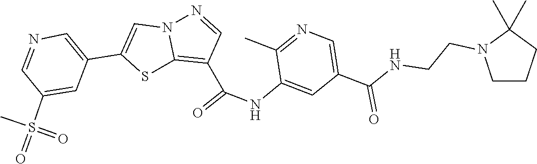 [SMILES: Cc1ncc(C(=O)NCCN2CCCC2(C)C)cc1NC(=O)c1cnn2cc(-c3cncc(S(C)(=O)=O)c3)sc12] | N-(5-((2-(2,2- dimethylpyrrolidin-1- yl)ethyl)carbamoyl)-2- methylpyridin-3-yl)-2-(5- (methylsulfonyl)pyridin-3- yl)pyrazolo[5,1-b]thiazole-7- carboxamide |
| 231 | 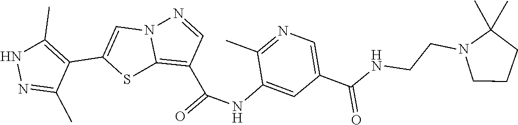 [SMILES: Cc1ncc(C(=O)NCCN2CCCC2(C)C)cc1NC(=O)c1cnn2cc(-c3c(C)n[nH]c3C)sc12] | 2-(3,5-Dimethyl-1H-pyrazol-4- yl)-N-(5-((2-(2,2- dimethylpyrrolidin-1- yl)ethyl)carbamoyl)-2- methylpyridin-3- yl)pyrazolo[5,1-b]thiazole-7- carboxamide |
| 232 | 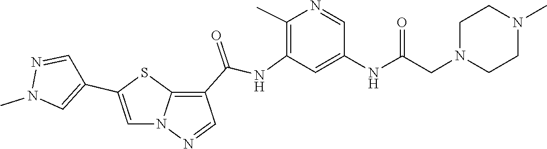 | 2-(1-methyl-1H-pyrazol-4-yl)- N-(2-methyl-5-(2-(4- methylpiperazin-1- yl)acetamido)pyridin-3- yl)pyrazolo[5,1-b]thiazole-7- carboxamide |
| 233 | 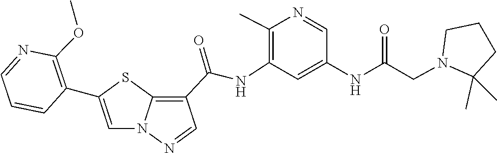 [SMILES: COc1ncccc1-c1cn2ncc(C(=O)Nc3cc(NC(=O)CN4CCCC4(C)C)cnc3C)c2s1] | N-(5-(2-(2,2-dimethylpyrrolidin- 1-yl)acetamido)-2- methylpyridin-3-yl)-2-(2- methoxypyridin-3- yl)pyrazolo[5,1-b]thiazole-7- carboxamide |
| 234 | 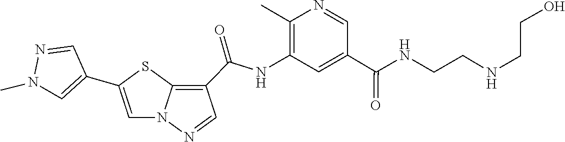 [SMILES: Cc1ncc(C(=O)NCCNCCO)cc1NC(=O)c1cnn2cc(-c3cnn(C)c3)sc12] | N-(5-((2-((2- hydroxyethyl)amino)ethyl) carbamoyl)-2-methylpyridin-3-yl)-2- (1-methyl-1H-pyrazol-4- yl)pyrazolo[5,1-b]thiazole-7- carboxamide |
| 235 | 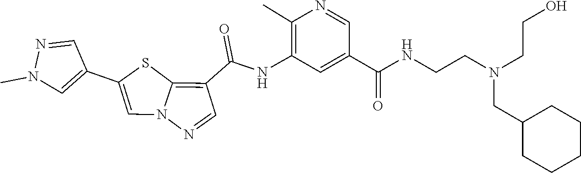 [SMILES: Cc1ncc(C(=O)NCCN(CCO)CC2CCCCC2)cc1NC(=O)c1cnn2cc(-c3cnn(C)c3)sc12] | N-(5-((2-((cyclohexylmethyl)(2- hydroxyethyl)amino)ethyl) carbamoyl)-2-methylpyridin-3-yl)-2- (1-methyl-1H-pyrazol-4- yl)pyrazolo[5,1-b]thiazole-7- carboxamide |
| 236 | 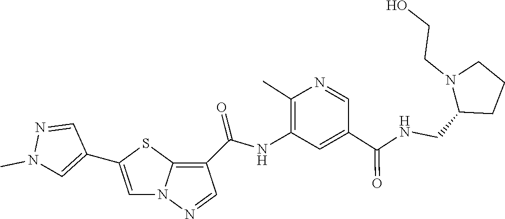 [SMILES: Cc1ncc(C(=O)NC[C@H]2CCCN2CCO)cc1NC(=O)c1cnn2cc(-c3cnn(C)c3)sc12] | (R)-N-(5-(((1-(2- hydroxyethyl)pyrrolidin-2- yl)methyl)carbamoyl)-2- methylpyridin-3-yl)-2-(1- methyl-1H-pyrazol-4- yl)pyrazolo[5,1-b]thiazole-7- carboxamide |
| 237 | 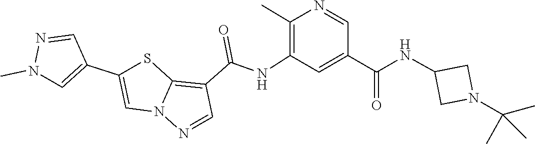 [SMILES: Cc1ncc(C(=O)NC2CN(C(C)(C)C)C2)cc1NC(=O)c1cnn2cc(-c3cnn(C)c3)sc12] | N-(5-((1-(tert-butyl)azetidin-3- yl)carbamoyl)-2-methylpyridin- 3-yl)-2-(1-methyl-1H-pyrazol-4- yl)pyrazolo[5,1-b]thiazole-7- carboxamide |
| 238 | 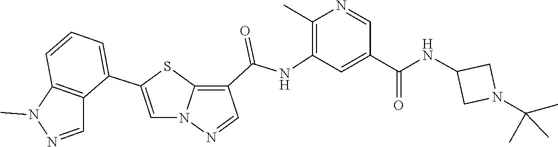 | N-(5-((1-(tert-butyl)azetidin-3- yl)carbamoyl)-2-methylpyridin- 3-yl)-2-(1-methyl-1H-indazol-4- yl)pyrazolo[5,1-b]thiazole-7- carboxamide |
| 239 | 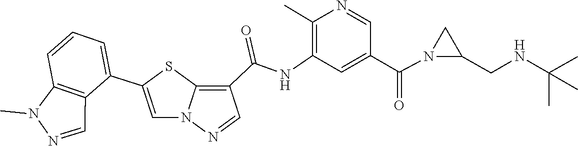 [SMILES: Cc1ncc(C(=O)N2CC2CNC(C)(C)C)cc1NC(=O)c1cnn2cc(-c3cccc4c3cnn4C)sc12] | N-(5-(2-((tert- butylamino)methyl)aziridine-1- carbonyl)-2-methylpyridin-3-yl)- 2-(1-methyl-1H-indazol-4- yl)pyrazolo[5,1-b]thiazole-7- carboxamide |
| 240 | 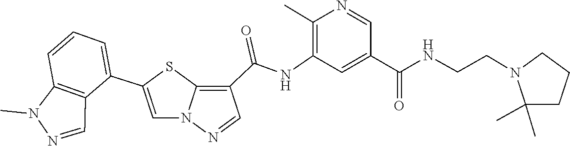 [SMILES: Cc1ncc(C(=O)NCCN2CCCC2(C)C)cc1NC(=O)c1cnn2cc(-c3cccc4c3cnn4C)sc12] | N-(5-((2-(2,2- dimethylpyrrolidin-1- yl)ethyl)carbamoyl)-2- methylpyridin-3-yl)-2-(1- methyl-1H-indazol-4- yl)pyrazolo[5,1-b]thiazole-7- carboxamide |
| 241 | 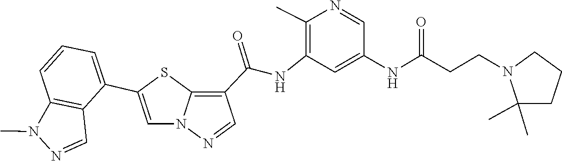 [SMILES: Cc1ncc(NC(=O)CCN2CCCC2(C)C)cc1NC(=O)c1cnn2cc(-c3cccc4c3cnn4C)sc12] | N-(5-(3-(2,2-dimethylpyrrolidin- 1-yl)propanamido)-2- methylpyridin-3-yl)-2-(1- methyl-1H-indazol-4- yl)pyrazolo[5,1-b]thiazole-7- carboxamide |
| 242 | 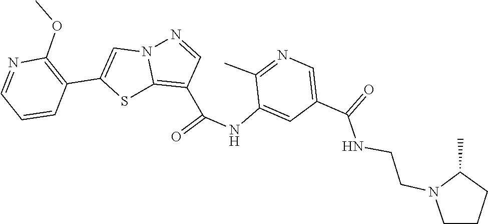 [SMILES: COc1ncccc1-c1cn2ncc(C(=O)Nc3cc(C(=O)NCCN4CCC[C@H]4C)cnc3C)c2s1] | (R)-2-(2-methoxypyridin-3-yl)- N-(2-methyl-5-((2-(2- methylpyrrolidin-1- yl)ethyl)carbamoyl)pyridin-3- yl)pyrazolo[5,1-b]thiazole-7- carboxamide |
| 243 | 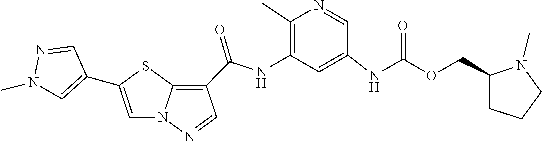 [SMILES: Cc1ncc(NC(=O)OC[C@@H]2CCCN2C)cc1NC(=O)c1cnn2cc(-c3cnn(C)c3)sc12] | (S)-(1-methylpyrrolidin-2- yl)methyl (6-methyl-5-(2-(1- methyl-1H-pyrazol-4- yl)pyrazolo[5,1-b]thiazole-7- carboxamido)pyridin-3- yl)carbamate |
| 244 | 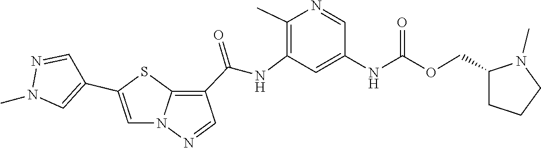 [SMILES: Cc1ncc(NC(=O)OC[C@H]2CCCN2C)cc1NC(=O)c1cnn2cc(-c3cnn(C)c3)sc12] | (R)-(1-methylpyrrolidin-2- yl)methyl (6-methyl-5-(2-(1- methyl-1H-pyrazol-4- yl)pyrazolo[5,1-b]thiazole-7- carboxamido)pyridin-3- yl)carbamate |
| 245 | 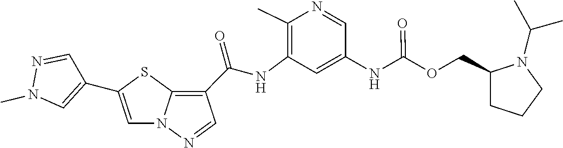 [SMILES: Cc1ncc(NC(=O)OC[C@@H]2CCCN2C(C)C)cc1NC(=O)c1cnn2cc(-c3cnn(C)c3)sc12] | (S)-(1-isopropylpyrrolidin-2- yl)methyl (6-methyl-5-(2-(1- methyl-1H-pyrazol-4- yl)pyrazolo[5,1-b]thiazole-7- carboxamido)pyridin-3- yl)carbamate |
| 246 | 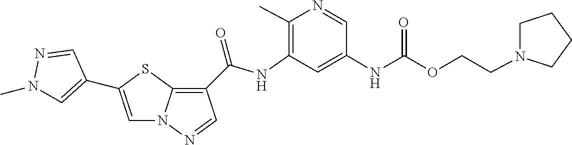 [SMILES: Cc1ncc(NC(=O)OCCN2CCCC2)cc1NC(=O)c1cnn2cc(-c3cnn(C)c3)sc12] | 2-(pyrrolidin-1-yl)ethyl (6- methyl-5-(2-(1-methyl-1H- pyrazol-4-yl)pyrazolo[5,1- b]thiazole-7- carboxamido)pyridin-3- yl)carbamate |
| 247 | 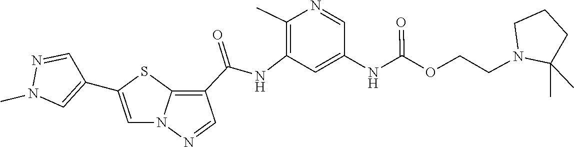 | 2-(2,2-dimethylpyrrolidin-1- yl)ethyl (6-methyl-5-(2-(1- methyl-1H-pyrazol-4- yl)pyrazolo[5,1-b]thiazole-7- carboxamido)pyridin-3- yl)carbamate |
| 248 | 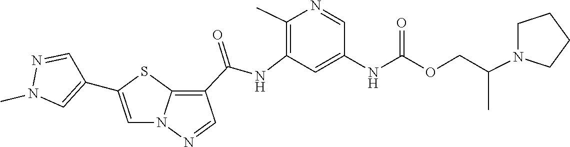 [SMILES: Cc1ncc(NC(=O)OCC(C)N2CCCC2)cc1NC(=O)c1cnn2cc(-c3cnn(C)c3)sc12] | 2-(pyrrolidin-1-yl)propyl (6- methyl-5-(2-(1-methyl-1H- pyrazol-4-yl)pyrazolo[5,1- b]thiazole-7- carboxamido)pyridin-3- yl)carbamate |
| 249 | 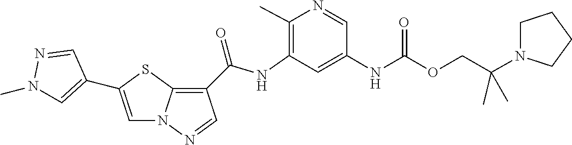 [SMILES: Cc1ncc(NC(=O)OCC(C)(C)N2CCCC2)cc1NC(=O)c1cnn2cc(-c3cnn(C)c3)sc12] | 2-methyl-2-(pyrrolidin-1- yl)propyl (6-methyl-5-(2-(1- methyl-1H-pyrazol-4- yl)pyrazolo[5,1-b]thiazole-7- carboxamido)pyridin-3- yl)carbamate |
| 250 | 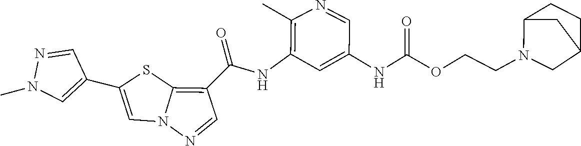 [SMILES: Cc1ncc(NC(=O)OCCN2CC3CCC2C3)cc1NC(=O)c1cnn2cc(-c3cnn(C)c3)sc12] | 2-(2-azabicyclo[2.2.1]heptan-2- yl)ethyl (6-methyl-5-(2-(1- methyl-1H-pyrazol-4- yl)pyrazolo[5,1-b]thiazole-7- carboxamido)pyridin-3- yl)carbamate |
| 251 | 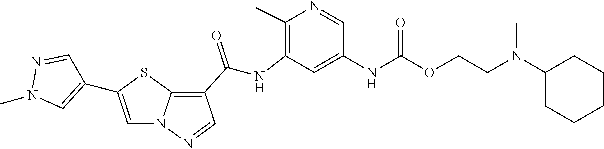 [SMILES: Cc1ncc(NC(=O)OCCN(C)C2CCCCC2)cc1NC(=O)c1cnn2cc(-c3cnn(C)c3)sc12] | 2- (cyclohexyl(methyl)amino)ethyl (6-methyl-5-(2-(1-methyl-1H- pyrazol-4-yl)pyrazolo[5,1- b]thiazole-7- carboxamido)pyridin-3- yl)carbamate |
| 252 | 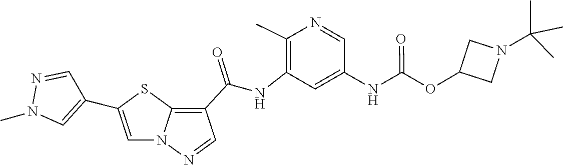 [SMILES: Cc1ncc(NC(=O)OC2CN(C(C)(C)C)C2)cc1NC(=O)c1cnn2cc(-c3cnn(C)c3)sc12] | 1-(tert-butyl)azetidin-3-yl (6- methyl-5-(2-(1-methyl-1H- pyrazol-4-yl)pyrazolo[5,1- b]thiazole-7- carboxamido)pyridin-3- yl)carbamate |
| 253 | 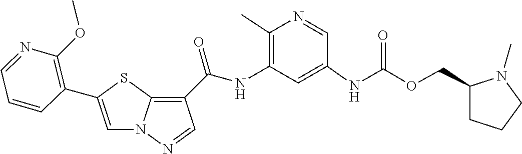 [SMILES: COc1ncccc1-c1cn2ncc(C(=O)Nc3cc(NC(=O)OC[C@@H]4CCCN4C)cnc3C)c2s1] | (S)-(1-methylpyrrolidin-2- yl)methyl (5-(2-(2- methoxypyridin-3- yl)pyrazolo[5,1-b]thiazole-7- carboxamido)-6-methylpyridin- 3-yl)carbamate |
| 254 | 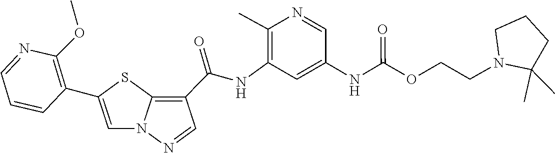 [SMILES: COc1ncccc1-c1cn2ncc(C(=O)Nc3cc(NC(=O)OCCN4CCCC4(C)C)cnc3C)c2s1] | 2-(2,2-dimethylpyrrolidin-1- yl)ethyl (5-(2-(2- methoxypyridin-3- yl)pyrazolo[5,1-b]thiazole-7- carboxamido)-6-methylpyridin- 3-yl)carbamate |
| 255 | 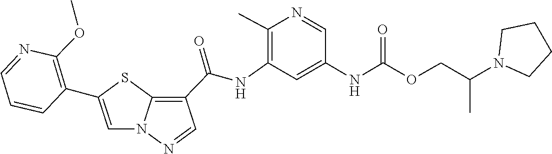 [SMILES: COc1ncccc1-c1cn2ncc(C(=O)Nc3cc(NC(=O)OCC(C)N4CCCC4)cnc3C)c2s1] | 2-(pyrrolidin-1-yl)propyl (5-(2- (2-methoxypyridin-3- yl)pyrazolo[5,1-b]thiazole-7- carboxamido)-6-methylpyridin- 3-yl)carbamate |
| 256 | 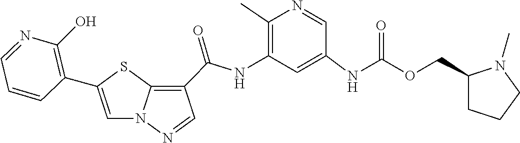 [SMILES: Cc1ncc(NC(=O)OC[C@@H]2CCCN2C)cc1NC(=O)c1cnn2cc(-c3cccnc3O)sc12] | (S)-(1-methylpyrrolidin-2- yl)methyl (5-(2-(2- hydroxypyridin-3- yl)pyrazolo[5,1-b]thiazole-7- carboxamido)-6-methylpyridin- 3-yl)carbamate |
| 257 | 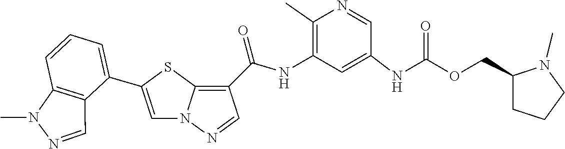 [SMILES: Cc1ncc(NC(=O)OC[C@@H]2CCCN2C)cc1NC(=O)c1cnn2cc(-c3cccc4c3cnn4C)sc12] | (S)-(1-methylpyrrolidin-2- yl)methyl (6-methyl-5-(2-(1- methyl-1H-indazol-4- yl)pyrazolo[5,1-b]thiazole-7- carboxamido)pyridin-3- yl)carbamate |
| 258 | 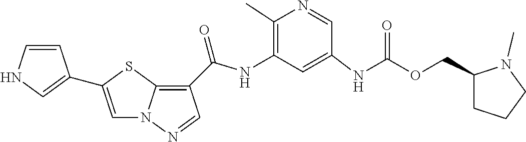 [SMILES: Cc1ncc(NC(=O)OC[C@@H]2CCCN2C)cc1NC(=O)c1cnn2cc(-c3cc[nH]c3)sc12] | (S)-(1-methylpyrrolidin-2- yl)methyl (5-(2-(1H-pyrrol-3- yl)pyrazolo[5,1-b]thiazole-7- carboxamido)-6-methylpyridin- 3-yl)carbamate |
| 259 | 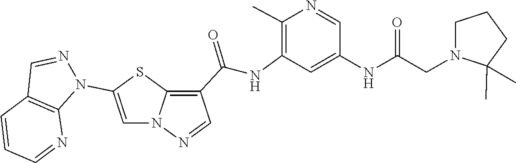 | N-(5-(2-(2,2-dimethylpyrrolidin- 1-yl)acetamido)-2- methylpyridin-3-yl)-2-(1H- pyrazolo[3,4-b]pyridin-1- yl)pyrazolo[5,1-b]thiazole-7- carboxamide |
| 260 | 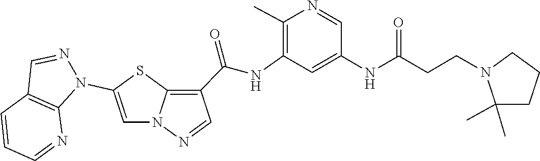 [SMILES: Cc1ncc(NC(=O)CCN2CCCC2(C)C)cc1NC(=O)c1cnn2cc(-n3ncc4cccnc43)sc12] | N-(5-(3-(2,2-dimethylpyrrolidin- 1-yl)propanamido)-2- methylpyridin-3-yl)-2-(1H- pyrazolo[3,4-b]pyridin-1- yl)pyrazolo[5,1-b]thiazole-7- carboxamide |
| 261 | 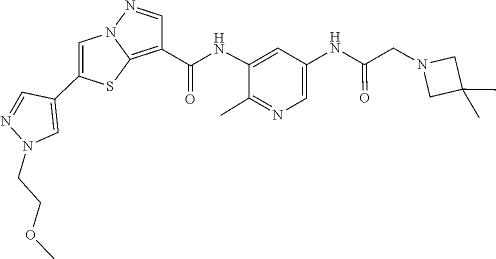 [SMILES: COCCn1cc(-c2cn3ncc(C(=O)Nc4cc(NC(=O)CN5CC(C)(C)C5)cnc4C)c3s2)cn1] | N-(5-(2-(3,3-dimethylazetidin-1- yl)acetamido)-2-methylpyridin- 3-yl)-2-(1-(2-methoxyethyl)-1H- pyrazol-4-yl)pyrazolo[5,1- b]thiazole-7-carboxamide |
| 262 | 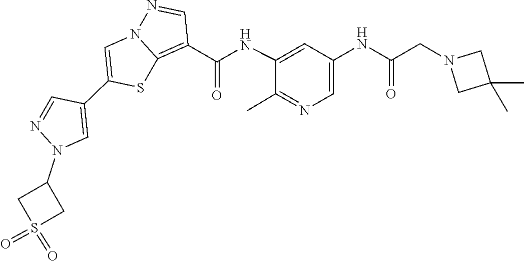 [SMILES: Cc1ncc(NC(=O)CN2CC(C)(C)C2)cc1NC(=O)c1cnn2cc(-c3cnn(C4CS(=O)(=O)C4)c3)sc12] | N-(5-(2-(3,3-dimethylazetidin-1- yl)acetamido)-2-methylpyridin- 3-yl)-2-(1-(1,1-dioxidothietan-3- yl)-1H-pyrazol-4- yl)pyrazolo[5,1-b]thiazole-7- carboxamide |
| 263 | 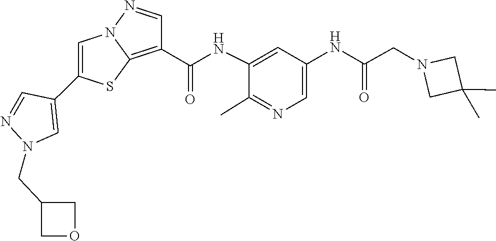 [SMILES: Cc1ncc(NC(=O)CN2CC(C)(C)C2)cc1NC(=O)c1cnn2cc(-c3cnn(CC4COC4)c3)sc12] | N-(5-(2-(3,3-dimethylazetidin-1- yl)acetamido)-2-methylpyridin- 3-yl)-2-(1-(oxetan-3-ylmethyl)- 1H-pyrazol-4-yl)pyrazolo[5,1- b]thiazole-7-carboxamide |
| 264 | 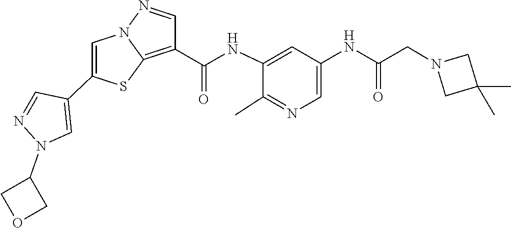 [SMILES: Cc1ncc(NC(=O)CN2CC(C)(C)C2)cc1NC(=O)c1cnn2cc(-c3cnn(C4COC4)c3)sc12] | N-(5-(2-(3,3-dimethylazetidin-1- yl)acetamido)-2-methylpyridin- 3-yl)-2-(1-(oxetan-3-yl)-1H- pyrazol-4-yl)pyrazolo[5,1- b]thiazole-7-carboxamide |
| 265 | 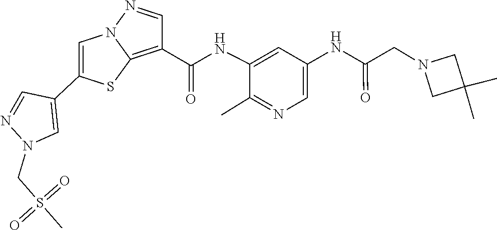 [SMILES: Cc1ncc(NC(=O)CN2CC(C)(C)C2)cc1NC(=O)c1cnn2cc(-c3cnn(CS(C)(=O)=O)c3)sc12] | N-(5-(2-(3,3-dimethylazetidin-1- yl)acetamido)-2-methylpyridin- 3-yl)-2-(1- ((methylsulfonyl)methyl)-1H- pyrazol-4-yl)pyrazolo[5,1- b]thiazole-7-carboxamide |
| 266 | 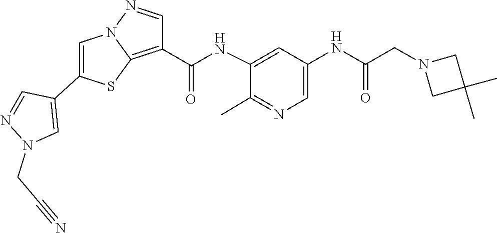 [SMILES: Cc1ncc(NC(=O)CN2CC(C)(C)C2)cc1NC(=O)c1cnn2cc(-c3cnn(CC#N)c3)sc12] | 2-(1-(cyanomethyl)-1H-pyrazol- 4-yl)-N-(5-(2-(3,3- dimethylazetidin-1- yl)acetamido)-2-methylpyridin- 3-yl)pyrazolo[5,1-b]thiazole-7- carboxamide |
| 267 | 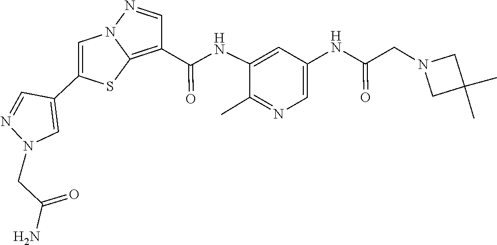 [SMILES: Cc1ncc(NC(=O)CN2CC(C)(C)C2)cc1NC(=O)c1cnn2cc(-c3cnn(CC(N)=O)c3)sc12] | 2-(1-(2-amino-2-oxoethyl)-1H- pyrazol-4-yl)-N-(5-(2-(3,3- dimethylazetidin-1- yl)acetamido)-2-methylpyridin- 3-yl)pyrazolo[5,1-b]thiazole-7- carboxamide |
| 268 | 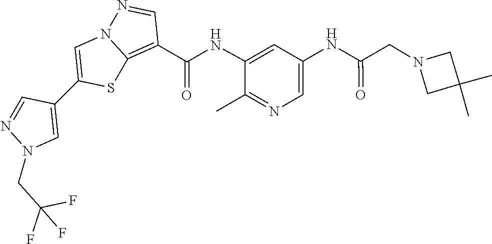 [SMILES: Cc1ncc(NC(=O)CN2CC(C)(C)C2)cc1NC(=O)c1cnn2cc(-c3cnn(CC(F)(F)F)c3)sc12] | N-(5-(2-(3,3-dimethylazetidin-1- yl)acetamido)-2-methylpyridin- 3-yl)-2-(1-(2,2,2-trifluoroethyl)- 1H-pyrazol-4-yl)pyrazolo[5,1- b]thiazole-7-carboxamide |
| 269 | 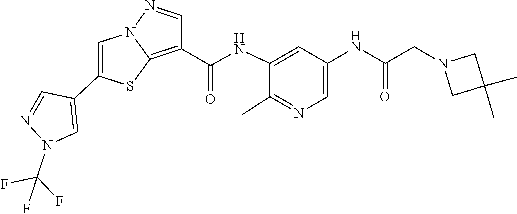 [SMILES: Cc1ncc(NC(=O)CN2CC(C)(C)C2)cc1NC(=O)c1cnn2cc(-c3cnn(C(F)(F)F)c3)sc12] | N-(5-(2-(3,3-dimethylazetidin-1- yl)acetamido)-2-methylpyridin- 3-yl)-2-(1-(trifluoromethyl)-1H- pyrazol-4-yl)pyrazolo[5,1- b]thiazole-7-carboxamide |
| 270 | 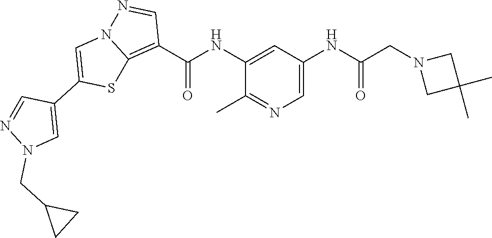 [SMILES: Cc1ncc(NC(=O)CN2CC(C)(C)C2)cc1NC(=O)c1cnn2cc(-c3cnn(CC4CC4)c3)sc12] | 2-(1-(cyclopropylmethyl)-1H- pyrazol-4-yl)-N-(5-(2-(3,3- dimethylazetidin-1- yl)acetamido)-2-methylpyridin- 3-yl)pyrazolo[5,1-b]thiazole-7- carboxamide |
| 271 | 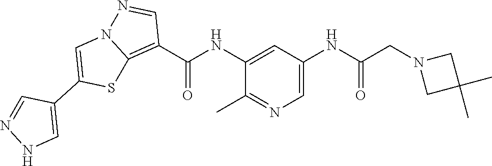 [SMILES: Cc1ncc(NC(=O)CN2CC(C)(C)C2)cc1NC(=O)c1cnn2cc(-c3cn[nH]c3)sc12] | N-(5-(2-(3,3-dimethylazetidin-1- yl)acetamido)-2-methylpyridin- 3-yl)-2-(1H-pyrazol-4- yl)pyrazolo[5,1-b]thiazole-7- carboxamide |
| 272 | 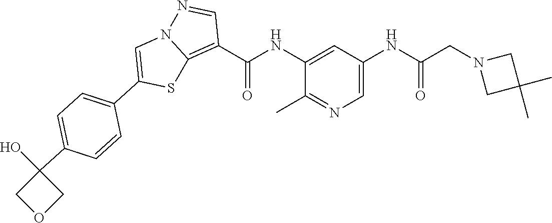 [SMILES: Cc1ncc(NC(=O)CN2CC(C)(C)C2)cc1NC(=O)c1cnn2cc(-c3ccc(C4(O)COC4)cc3)sc12] | N-(5-(2-(3,3-dimethylazetidin-1- yl)acetamido)-2-methylpyridin- 3-yl)-2-(4-(3-hydroxyoxetan-3- yl)phenyl)pyrazolo[5,1- b]thiazole-7-carboxamide |
| 273 | 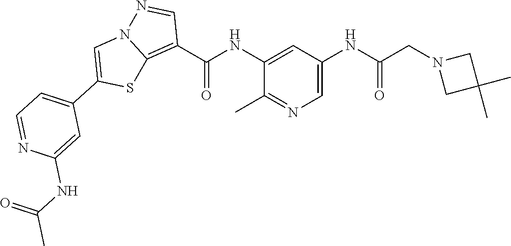 [SMILES: CC(=O)Nc1cc(-c2cn3ncc(C(=O)Nc4cc(NC(=O)CN5CC(C)(C)C5)cnc4C)c3s2)ccn1] | 2-(2-acetamidopyridin-4-yl)-N- (5-(2-(3,3-dimethylazetidin-1- yl)acetamido)-2-methylpyridin- 3-yl)pyrazolo[5,1-b]thiazole-7- carboxamide |
| 274 | 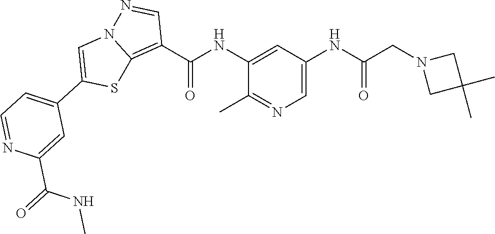 [SMILES: CNC(=O)c1cc(-c2cn3ncc(C(=O)Nc4cc(NC(=O)CN5CC(C)(C)C5)cnc4C)c3s2)ccn1] | N-(5-(2-(3,3-dimethylazetidin-1- yl)acetamido)-2-methylpyridin- 3-yl)-2-(2- (methylcarbamoyl)pyridin-4- yl)pyrazolo[5,1-b]thiazole-7- carboxamide |
| 275 | 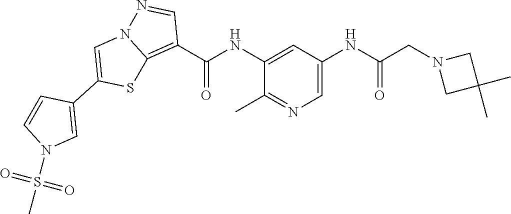 [SMILES: Cc1ncc(NC(=O)CN2CC(C)(C)C2)cc1NC(=O)c1cnn2cc(-c3ccn(S(C)(=O)=O)c3)sc12] | N-(5-(2-(3,3-dimethylazetidin-1- yl)acetamido)-2-methylpyridin- 3-yl)-2-(1-(methylsulfonyl)-1H- pyrrol-3-yl)pyrazolo[5,1- b]thiazole-7-carboxamide |
| 277 | 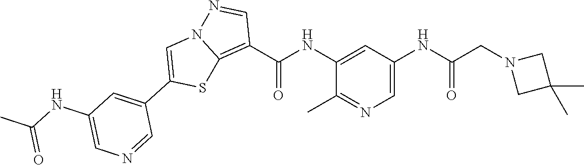 [SMILES: CC(=O)Nc1cncc(-c2cn3ncc(C(=O)Nc4cc(NC(=O)CN5CC(C)(C)C5)cnc4C)c3s2)c1] | 2-(5-acetamidopyridin-3-yl)-N- (5-(2-(3,3-dimethylazetidin-1- yl)acetamido)-2-methylpyridin- 3-yl)pyrazolo[5,1-b]thiazole-7- carboxamide |
| 278 | 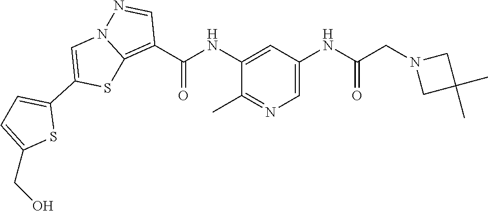 [SMILES: Cc1ncc(NC(=O)CN2CC(C)(C)C2)cc1NC(=O)c1cnn2cc(-c3ccc(CO)s3)sc12] | N-(5-(2-(3,3-dimethylazetidin-1- yl)acetamido)-2-methylpyridin- 3-yl)-2-(5- (hydroxymethyl)thiophen-2- yl)pyrazolo[5,1-b]thiazole-7- carboxamide |
| 279 | 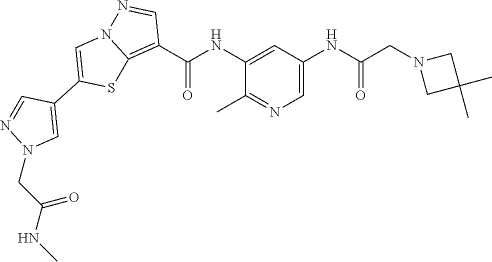 [SMILES: CNC(=O)Cn1cc(-c2cn3ncc(C(=O)Nc4cc(NC(=O)CN5CC(C)(C)C5)cnc4C)c3s2)cn1] | N-(5-(2-(3,3-dimethylazetidin-1- yl)acetamido)-2-methylpyridin- 3-yl)-2-(1-(2-(methylamino)-2- oxoethyl)-1H-pyrazol-4- yl)pyrazolo[5,1-b]thiazole-7- carboxamide |
| 280 | 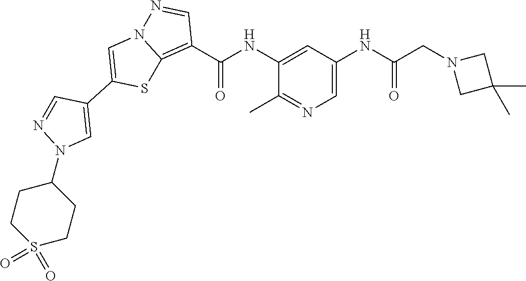 [SMILES: Cc1ncc(NC(=O)CN2CC(C)(C)C2)cc1NC(=O)c1cnn2cc(-c3cnn(C4CCS(=O)(=O)CC4)c3)sc12] | N-(5-(2-(3,3-dimethylazetidin-1- yl)acetamido)-2-methylpyridin- 3-yl)-2-(1-(1,1- dioxidotetrahydro-2H-thiopyran- 4-yl)-1H-pyrazol-4- yl)pyrazolo[5,1-b]thiazole-7- carboxamide |
| 281 | 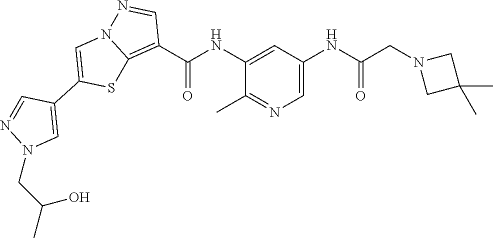 [SMILES: Cc1ncc(NC(=O)CN2CC(C)(C)C2)cc1NC(=O)c1cnn2cc(-c3cnn(CC(C)O)c3)sc12] | N-(5-(2-(3,3-dimethylazetidin-1- yl)acetamido)-2-methylpyridin- 3-yl)-2-(1-(2-hydroxypropyl)- 1H-pyrazol-4-yl)pyrazolo[5,1- b]thiazole-7-carboxamide |
| 282 | 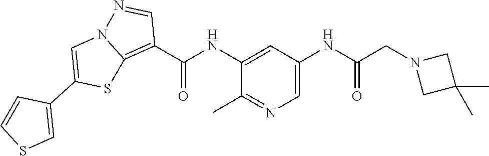 [SMILES: Cc1ncc(NC(=O)CN2CC(C)(C)C2)cc1NC(=O)c1cnn2cc(-c3ccsc3)sc12] | N-(5-(2-(3,3-dimethylazetidin-1- yl)acetamido)-2-methylpyridin- 3-yl)-2-(thiophen-3- yl)pyrazolo[5,1-b]thiazole-7- carboxamide |
| 283 | 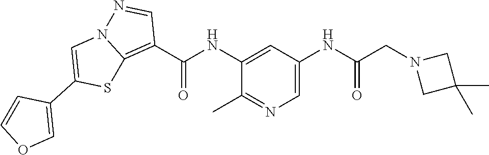 [SMILES: Cc1ncc(NC(=O)CN2CC(C)(C)C2)cc1NC(=O)c1cnn2cc(-c3ccoc3)sc12] | N-(5-(2-(3,3-dimethylazetidin-1- yl)acetamido)-2-methylpyridin- 3-yl)-2-(furan-3-yl)pyrazolo[5,1- b]thiazole-7-carboxamide |
| 284 | 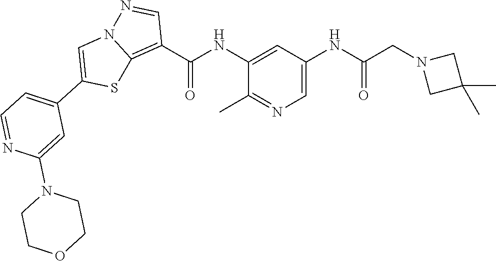 [SMILES: Cc1ncc(NC(=O)CN2CC(C)(C)C2)cc1NC(=O)c1cnn2cc(-c3ccnc(N4CCOCC4)c3)sc12] | N-(5-(2-(3,3-dimethylazetidin-1- yl)acetamido)-2-methylpyridin- 3-yl)-2-(2-morpholinopyridin-4- yl)pyrazolo[5,1-b]thiazole-7- carboxamide |
| 285 | 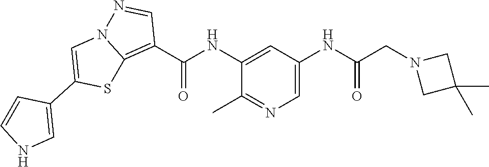 [SMILES: Cc1ncc(NC(=O)CN2CC(C)(C)C2)cc1NC(=O)c1cnn2cc(-c3cc[nH]c3)sc12] | N-(5-(2-(3,3-dimethylazetidin-1- yl)acetamido)-2-methylpyridin- 3-yl)-2-(1H-pyrrol-3- yl)pyrazolo[5,1-b]thiazole-7- carboxamide |
| 286 | 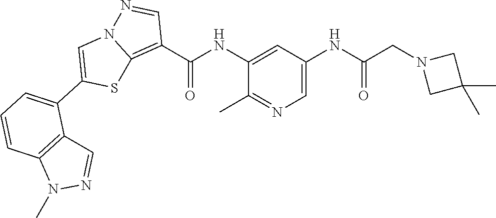 [SMILES: Cc1ncc(NC(=O)CN2CC(C)(C)C2)cc1NC(=O)c1cnn2cc(-c3cccc4c3cnn4C)sc12] | N-(5-(2-(3,3-dimethylazetidin-1- yl)acetamido)-2-methylpyridin- 3-yl)-2-(1-methyl-1H-indazol-4- yl)pyrazolo[5,1-b]thiazole-7- carboxamide |
| 287 | 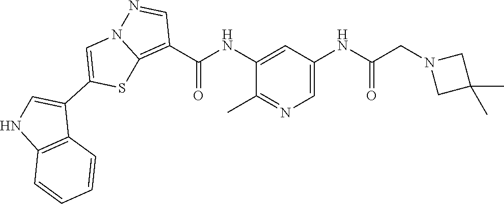 [SMILES: Cc1ncc(NC(=O)CN2CC(C)(C)C2)cc1NC(=O)c1cnn2cc(-c3c[nH]c4ccccc34)sc12] | N-(5-(2-(3,3-dimethylazetidin-1- yl)acetamido)-2-methylpyridin- 3-yl)-2-(1H-indol-3- yl)pyrazolo[5,1-b]thiazole-7- carboxamide |
| 288 | 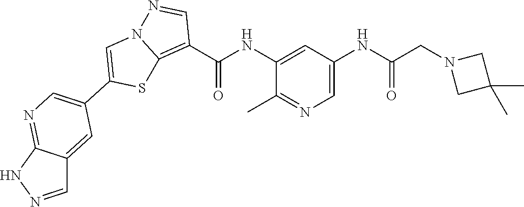 [SMILES: Cc1ncc(NC(=O)CN2CC(C)(C)C2)cc1NC(=O)c1cnn2cc(-c3cnc4[nH]ncc4c3)sc12] | N-(5-(2-(3,3-dimethylazetidin-1- yl)acetamido)-2-methylpyridin- 3-yl)-2-(1H-pyrazolo[3,4- b]pyridin-5-yl)pyrazolo[5,1- b]thiazole-7-carboxamide |
| 289 | 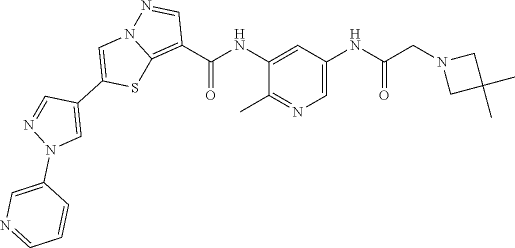 [SMILES: Cc1ncc(NC(=O)CN2CC(C)(C)C2)cc1NC(=O)c1cnn2cc(-c3cnn(-c4cccnc4)c3)sc12] | N-(5-(2-(3,3-dimethylazetidin-1- yl)acetamido)-2-methylpyridin- 3-yl)-2-(1-(pyridin-3-yl)-1H- pyrazol-4-yl)pyrazolo[5,1- b]thiazole-7-carboxamide |
| 290 | 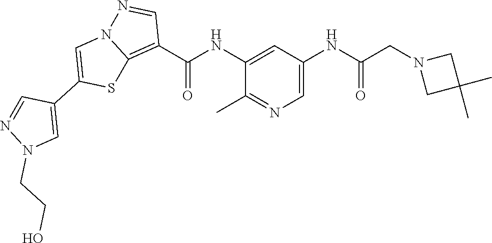 [SMILES: Cc1ncc(NC(=O)CN2CC(C)(C)C2)cc1NC(=O)c1cnn2cc(-c3cnn(CCO)c3)sc12] | N-(5-(2-(3,3-dimethylazetidin-1- yl)acetamido)-2-methylpyridin- 3-yl)-2-(1-(2-hydroxyethyl)-1H- pyrazol-4-yl)pyrazolo[5,1- b]thiazole-7-carboxamide |
| 292 | 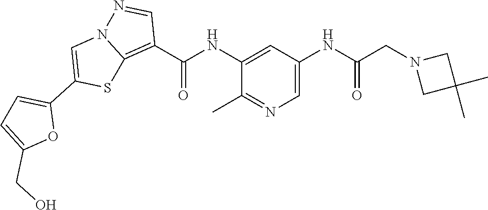 [SMILES: Cc1ncc(NC(=O)CN2CC(C)(C)C2)cc1NC(=O)c1cnn2cc(-c3ccc(CO)o3)sc12] | N-(5-(2-(3,3-dimethylazetidin-1- yl)acetamido)-2-methylpyridin- 3-yl)-2-(5- (hydroxymethyl)furan-2- yl)pyrazolo[5,1-b]thiazole-7- carboxamide |
| 293 | 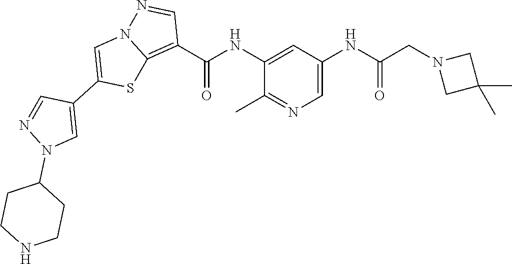 [SMILES: Cc1ncc(NC(=O)CN2CC(C)(C)C2)cc1NC(=O)c1cnn2cc(-c3cnn(C4CCNCC4)c3)sc12] | N-(5-(2-(3,3-dimethylazetidin-1- yl)acetamido)-2-methylpyridin- 3-yl)-2-(1-(piperidin-4-yl)-1H- pyrazol-4-yl)pyrazolo[5,1- b]thiazole-7-carboxamide |
| 294 | 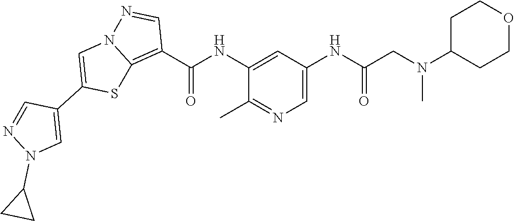 [SMILES: Cc1ncc(NC(=O)CN(C)C2CCOCC2)cc1NC(=O)c1cnn2cc(-c3cnn(C4CC4)c3)sc12] | 2-(1-cyclopropyl-1H-pyrazol-4- yl)-N-(2-methyl-5-(2- (methyl(tetrahydro-2H-pyran-4- yl)amino)acetamido)pyridin-3- yl)pyrazolo[5,1-b]thiazole-7- carboxamide |
| 295 | 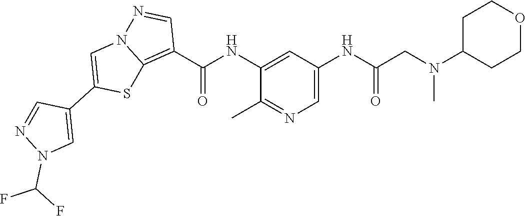 [SMILES: Cc1ncc(NC(=O)CN(C)C2CCOCC2)cc1NC(=O)c1cnn2cc(-c3cnn(C(F)F)c3)sc12] | 2-(1-(difluoromethyl)-1H- pyrazol-4-yl)-N-(2-methyl-5-(2- (methyl(tetrahydro-2H-pyran-4- yl)amino)acetamido)pyridin-3- yl)pyrazolo[5,1-b]thiazole-7- carboxamide |
| 296 | 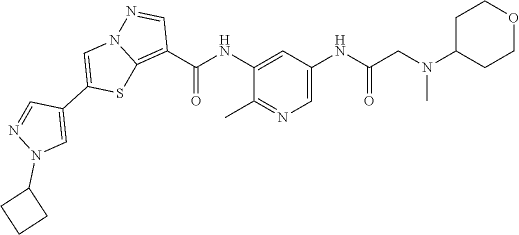 [SMILES: Cc1ncc(NC(=O)CN(C)C2CCOCC2)cc1NC(=O)c1cnn2cc(-c3cnn(C4CCC4)c3)sc12] | 2-(1-cyclobutyl-1H-pyrazol-4- yl)-N-(2-methyl-5-(2- (methyl(tetrahydro-2H-pyran-4- yl)amino)acetamido)pyridin-3- yl)pyrazolo[5,1-b]thiazole-7- carboxamide |
| 297 | 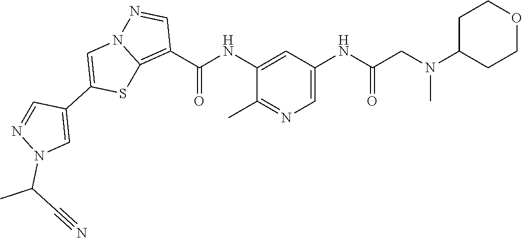 [SMILES: Cc1ncc(NC(=O)CN(C)C2CCOCC2)cc1NC(=O)c1cnn2cc(-c3cnn(C(C)C#N)c3)sc12] | 2-(1-(1-cyanoethyl)-1H-pyrazol- 4-yl)-N-(2-methyl-5-(2- (methyl(tetrahydro-2H-pyran-4- yl)amino)acetamido)pyridin-3- yl)pyrazolo[5,1-b]thiazole-7- carboxamide |
| 298 | 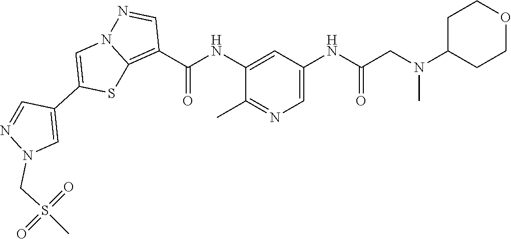 [SMILES: Cc1ncc(NC(=O)CN(C)C2CCOCC2)cc1NC(=O)c1cnn2cc(-c3cnn(CS(C)(=O)=O)c3)sc12] | N-(2-methyl-5-(2- (methyl(tetrahydro-2H-pyran-4- yl)amino)acetamido)pyridin-3- yl)-2-(1- ((methylsulfonyl)methyl)-1H- pyrazol-4-yl)pyrazolo[5,1- b]thiazole-7-carboxamide |
| 299 | 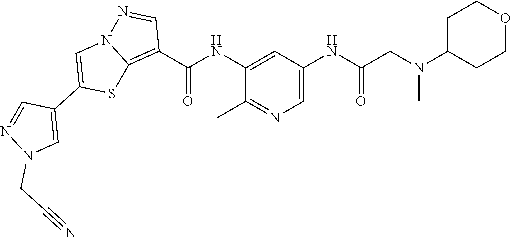 [SMILES: Cc1ncc(NC(=O)CN(C)C2CCOCC2)cc1NC(=O)c1cnn2cc(-c3cnn(CC#N)c3)sc12] | 2-(1-(cyanomethyl)-1H-pyrazol- 4-yl)-N-(2-methyl-5-(2- (methyl(tetrahydro-2H-pyran-4- yl)amino)acetamido)pyridin-3- yl)pyrazolo[5,1-b]thiazole-7- carboxamide |
| 300 | 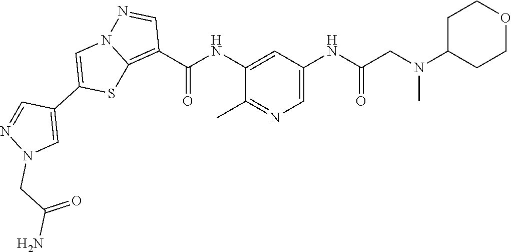 [SMILES: Cc1ncc(NC(=O)CN(C)C2CCOCC2)cc1NC(=O)c1cnn2cc(-c3cnn(CC(N)=O)c3)sc12] | 2-(1-(2-amino-2-oxoethyl)-1H- pyrazol-4-yl)-N-(2-methyl-5-(2- (methyl(tetrahydro-2H-pyran-4- yl)amino)acetamido)pyridin-3- yl)pyrazolo[5,1-b]thiazole-7- carboxamide |
| 301 | 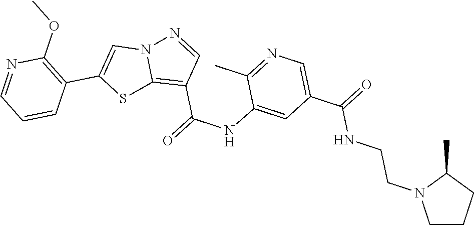 [SMILES: COc1ncccc1-c1cn2ncc(C(=O)Nc3cc(C(=O)NCCN4CCC[C@@H]4C)cnc3C)c2s1] | (S)-2-(2-methoxypyridin-3-yl)- N-(2-methyl-5-((2-(2- methylpyrrolidin-1- yl)ethyl)carbamoyl)pyridin-3- yl)pyrazolo[5,1-b]thiazole-7- carboxamide |
| 302 | 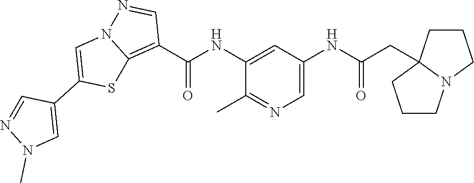 [SMILES: Cc1ncc(NC(=O)CC23CCCN2CCC3)cc1NC(=O)c1cnn2cc(-c3cnn(C)c3)sc12] | 2-(1-methyl-1H-pyrazol-4-yl)-N- (2-methyl-5-(2-(tetrahydro-1H- pyrrolizin-7a(5H)- yl)acetamido)pyridin-3- yl)pyrazolo[5,1-b]thiazole-7- carboxamide |
| 303 | 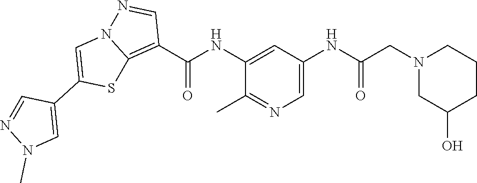 [SMILES: Cc1ncc(NC(=O)CN2CCCC(O)C2)cc1NC(=O)c1cnn2cc(-c3cnn(C)c3)sc12] | N-(5-(2-(3-hydroxypiperidin-1- yl)acetamido)-2-methylpyridin- 3-yl)-2-(1-methyl-1H-pyrazol-4- yl)pyrazolo[5,1-b]thiazole-7- carboxamide |
| 304 | 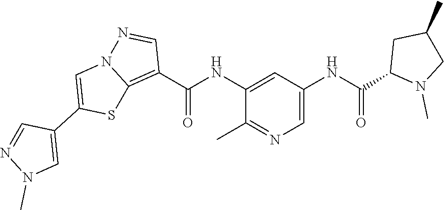 [SMILES: Cc1ncc(NC(=O)[C@@H]2C[C@@H](C)CN2C)cc1NC(=O)c1cnn2cc(-c3cnn(C)c3)sc12] | N-(5-((2S,4R)-1,4- dimethylpyrrolidine-2- carboxamido)-2-methylpyridin- 3-yl)-2-(1-methyl-1H-pyrazol-4- yl)pyrazolo[5,1-b]thiazole-7- carboxamide |
| 305 | 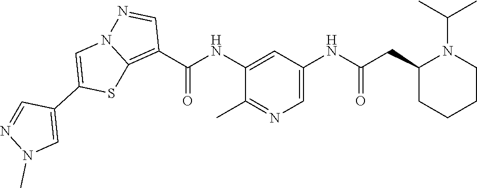 [SMILES: Cc1ncc(NC(=O)C[C@@H]2CCCCN2C(C)C)cc1NC(=O)c1cnn2cc(-c3cnn(C)c3)sc12] | (S)-N-(5-(2-(1- isopropylpiperidin-2- yl)acetamido)-2-methylpyridin- 3-yl)-2-(1-methyl-1H-pyrazol-4- yl)pyrazolo[5,1-b]thiazole-7- carboxamide |
| 306 | 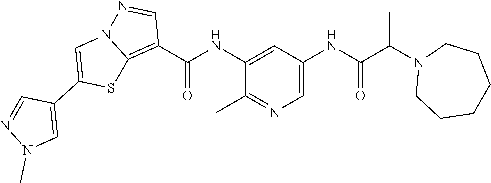 [SMILES: Cc1ncc(NC(=O)C(C)N2CCCCCC2)cc1NC(=O)c1cnn2cc(-c3cnn(C)c3)sc12] | N-(5-(2-(azepan-1- yl)propanamido)-2- methylpyridin-3-yl)-2-(1- methyl-1H-pyrazol-4- yl)pyrazolo[5,1-b]thiazole-7- carboxamide |
| 307 | 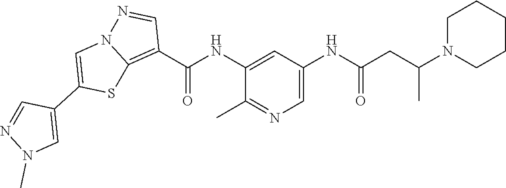 [SMILES: Cc1ncc(NC(=O)CC(C)N2CCCCC2)cc1NC(=O)c1cnn2cc(-c3cnn(C)c3)sc12] | 2-(1-methyl-1H-pyrazol-4-yl)-N- (2-methyl-5-(3-(piperidin-1- yl)butanamido)pyridin-3- yl)pyrazolo[5,1-b]thiazole-7- carboxamide |
| 308 | 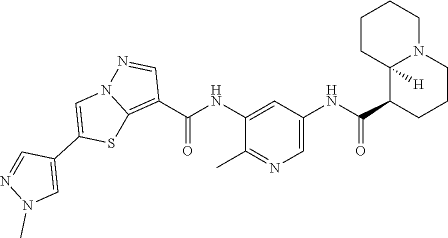 | 2-(1-methyl-1H-pyrazol-4-yl)-N- (2-methyl-5-((1R,9aR)- octahydro-2H-quinolizine-1- carboxamido)pyridin-3- yl)pyrazolo[5,1-b]thiazole-7- carboxamide |
| 309 | 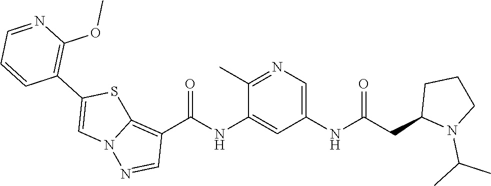 [SMILES: COc1ncccc1-c1cn2ncc(C(=O)Nc3cc(NC(=O)C[C@H]4CCCN4C(C)C)cnc3C)c2s1] | (R)-N-(5-(2-(1- isopropylpyrrolidin-2- yl)acetamido)-2-methylpyridin- 3-yl)-2-(2-methoxypyridin-3- yl)pyrazolo[5,1-b]thiazole-7- carboxamide |
| 310 | 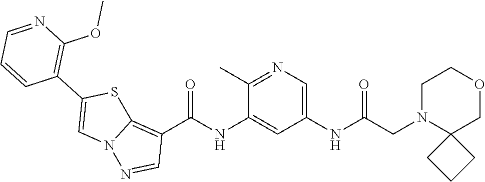 [SMILES: COc1ncccc1-c1cn2ncc(C(=O)Nc3cc(NC(=O)CN4CCOCC45CCC5)cnc3C)c2s1] | N-(5-(2-(8-oxa-5- azaspiro[3.5]nonan-5- yl)acetamido)-2-methylpyridin- 3-yl)-2-(2-methoxypyridin-3- yl)pyrazolo[5,1-b]thiazole-7- carboxamide |
| 311 | 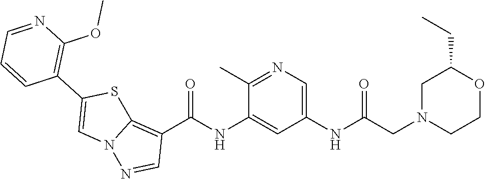 [SMILES: CC[C@H]1CN(CC(=O)Nc2cnc(C)c(NC(=O)c3cnn4cc(-c5cccnc5OC)sc34)c2)CCO1] | (S)-N-(5-(2-(2- ethylmorpholino)acetamido)-2- methylpyridin-3-yl)-2-(2- methoxypyridin-3- yl)pyrazolo[5,1-b]thiazole-7- carboxamide |
| 312 | 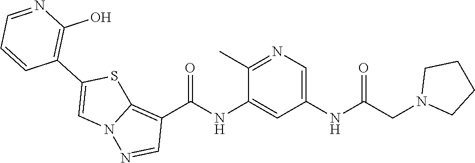 [SMILES: Cc1ncc(NC(=O)CN2CCCC2)cc1NC(=O)c1cnn2cc(-c3cccnc3O)sc12] | 2-(2-hydroxypyridin-3-yl)-N-(2- methyl-5-(2-(pyrrolidin-1- yl)acetamido)pyridin-3- yl)pyrazolo[5,1-b]thiazole-7- carboxamide |
| 313 | 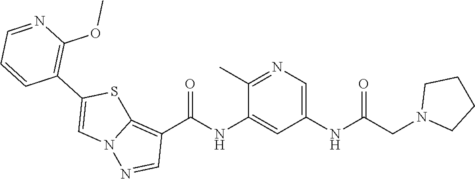 [SMILES: COc1ncccc1-c1cn2ncc(C(=O)Nc3cc(NC(=O)CN4CCCC4)cnc3C)c2s1] | 2-(2-methoxypyridin-3-yl)-N-(2- methyl-5-(2-(pyrrolidin-1- yl)acetamido)pyridin-3- yl)pyrazolo[5,1-b]thiazole-7- carboxamide |
| 314 | 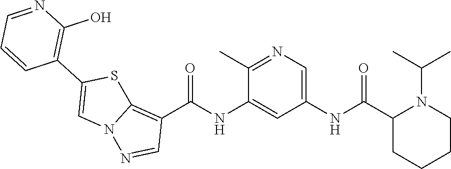 [SMILES: Cc1ncc(NC(=O)C2CCCCN2C(C)C)cc1NC(=O)c1cnn2cc(-c3cccnc3O)sc12] | 2-(2-hydroxypyridin-3-yl)-N-(5- (1-isopropylpiperidine-2- carboxamido)-2-methylpyridin- 3-yl)pyrazolo[5,1-b]thiazole-7- carboxamide |
| 315 | 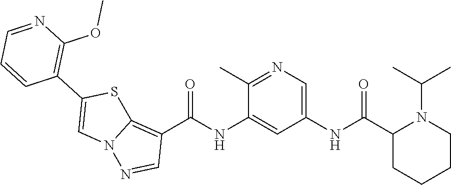 [SMILES: COc1ncccc1-c1cn2ncc(C(=O)Nc3cc(NC(=O)C4CCCCN4C(C)C)cnc3C)c2s1] | N-(5-(1-isopropylpiperidine-2- carboxamido)-2-methylpyridin- 3-yl)-2-(2-methoxypyridin-3- yl)pyrazolo[5,1-b]thiazole-7- carboxamide |
| 316 | 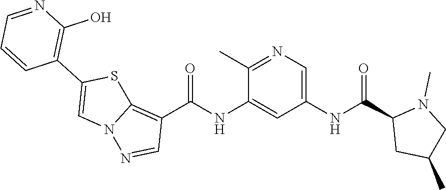 [SMILES: Cc1ncc(NC(=O)[C@@H]2C[C@H](C)CN2C)cc1NC(=O)c1cnn2cc(-c3cccnc3O)sc12] | N-(5-((2S,4S)-1,4- dimethylpyrrolidine-2- carboxamido)-2-methylpyridin- 3-yl)-2-(2-hydroxypyridin-3- yl)pyrazolo[5,1-b]thiazole-7- carboxamide |
| 317 | 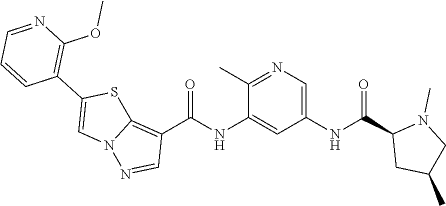 [SMILES: COc1ncccc1-c1cn2ncc(C(=O)Nc3cc(NC(=O)[C@@H]4C[C@H](C)CN4C)cnc3C)c2s1] | N-(5-((2S,4S)-1,4- dimethylpyrrolidine-2- carboxamido)-2-methylpyridin- 3-yl)-2-(2-methoxypyridin-3- yl)pyrazolo[5,1-b]thiazole-7- carboxamide |
| 318 | 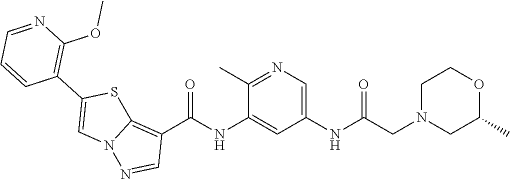 [SMILES: COc1ncccc1-c1cn2ncc(C(=O)Nc3cc(NC(=O)CN4CCO[C@H](C)C4)cnc3C)c2s1] | (R)-2-(2-methoxypyridin-3-yl)- N-(2-methyl-5-(2-(2- methylmorpholino)acetamido) pyridin-3-yl)pyrazolo[5,1- b]thiazole-7-carboxamide |
| 319 | 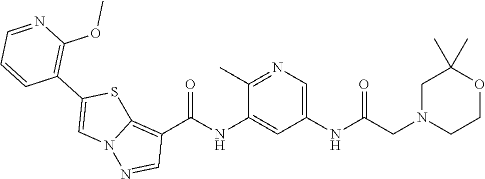 [SMILES: COc1ncccc1-c1cn2ncc(C(=O)Nc3cc(NC(=O)CN4CCOC(C)(C)C4)cnc3C)c2s1] | N-(5-(2-(2,2- dimethylmorpholino)acetamido)- 2-methylpyridin-3-yl)-2-(2- methoxypyridin-3- yl)pyrazolo[5,1-b]thiazole-7- carboxamide |
| 320 | 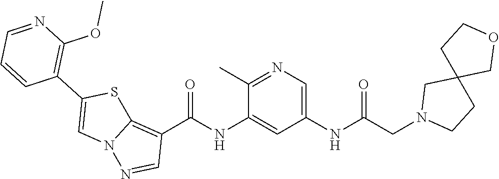 [SMILES: COc1ncccc1-c1cn2ncc(C(=O)Nc3cc(NC(=O)CN4CCC5(CCOC5)C4)cnc3C)c2s1] | N-(5-(2-(2-oxa-7- azaspiro[4.4]nonan-7- yl)acetamido)-2-methylpyridin- 3-yl)-2-(2-methoxypyridin-3- yl)pyrazolo[5,1-b]thiazole-7- carboxamide |
| 321 | 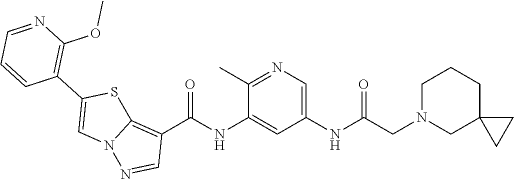 [SMILES: COc1ncccc1-c1cn2ncc(C(=O)Nc3cc(NC(=O)CN4CCCC5(CC5)C4)cnc3C)c2s1] | N-(5-(2-(5-azaspiro[2.5]octan-5- yl)acetamido)-2-methylpyridin- 3-yl)-2-(2-methoxypyridin-3- yl)pyrazolo[5,1-b]thiazole-7- carboxamide |
| 322 | 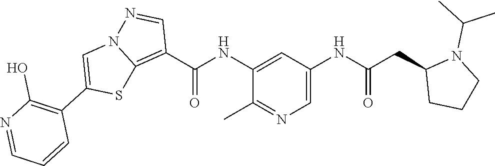 [SMILES: Cc1ncc(NC(=O)C[C@@H]2CCCN2C(C)C)cc1NC(=O)c1cnn2cc(-c3cccnc3O)sc12] | (S)-2-(2-hydroxypyridin-3-yl)- N-(5-(2-(1-isopropylpyrrolidin- 2-yl)acetamido)-2- methylpyridin-3- yl)pyrazolo[5,1-b]thiazole-7- carboxamide |
| 323 | 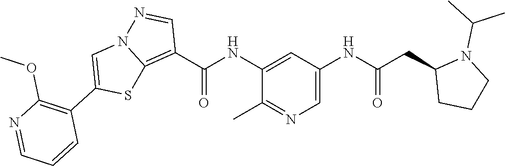 [SMILES: COc1ncccc1-c1cn2ncc(C(=O)Nc3cc(NC(=O)C[C@@H]4CCCN4C(C)C)cnc3C)c2s1] | (S)-N-(5-(2-(1- isopropylpyrrolidin-2- yl)acetamido)-2-methylpyridin- 3-yl)-2-(2-methoxypyridin-3- yl)pyrazolo[5,1-b]thiazole-7- carboxamide |
| 324 | 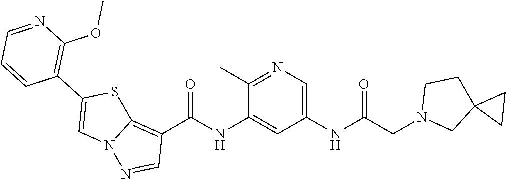 [SMILES: COc1ncccc1-c1cn2ncc(C(=O)Nc3cc(NC(=O)CN4CCC5(CC5)C4)cnc3C)c2s1] | N-(5-(2-(5-azaspiro[2.4]heptan- 5-yl)acetamido)-2- methylpyridin-3-yl)-2-(2- methoxypyridin-3- yl)pyrazolo[5,1-b]thiazole-7- carboxamide |
| 325 | 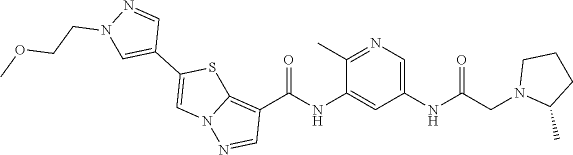 [SMILES: COCCn1cc(-c2cn3ncc(C(=O)Nc4cc(NC(=O)CN5CCC[C@@H]5C)cnc4C)c3s2)cn1] | (S)-2-(1-(2-methoxyethyl)-1H- pyrazol-4-yl)-N-(2-methyl-5-(2- (2-methylpyrrolidin-1- yl)acetamido)pyridin-3- yl)pyrazolo[5,1-b]thiazole-7- carboxamide |
| 326 | 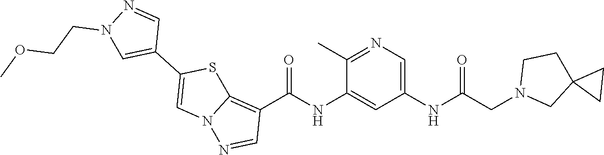 [SMILES: COCCn1cc(-c2cn3ncc(C(=O)Nc4cc(NC(=O)CN5CCC6(CC6)C5)cnc4C)c3s2)cn1] | N-(5-(2-(5-azaspiro[2.4]heptan- 5-yl)acetamido)-2- methylpyridin-3-yl)-2-(1-(2- methoxyethyl)-1H-pyrazol-4- yl)pyrazolo[5,1-b]thiazole-7- carboxamide |
| 327 | 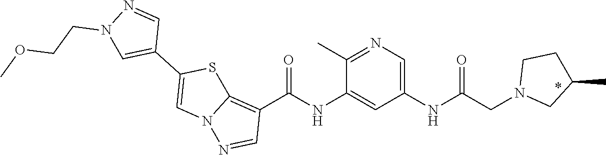 | (R*)-2-(1-(2-methoxyethyl)-1H- pyrazol-4-yl)-N-(2-methyl-5-(2- (3-methylpyrrolidin-1- yl)acetamido)pyridin-3- yl)pyrazolo[5,1-b]thiazole-7- carboxamide |
| 328 | 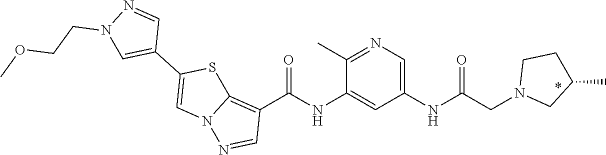 | (S*)-2-(1-(2-methoxyethyl)-1H- pyrazol-4-yl)-N-(2-methyl-5-(2- (3-methylpyrrolidin-1- yl)acetamido)pyridin-3- yl)pyrazolo[5,1-b]thiazole-7- carboxamide |
| 329 | 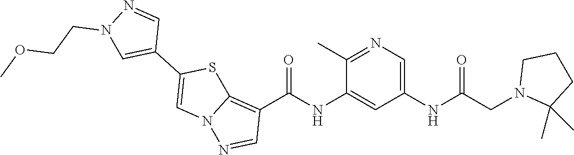 | N-(5-(2-(2,2-dimethylpyrrolidin- 1-yl)acetamido)-2- methylpyridin-3-yl)-2-(1-(2- methoxyethyl)-1H-pyrazol-4- yl)pyrazolo[5,1-b]thiazole-7- carboxamide |
| 330 | 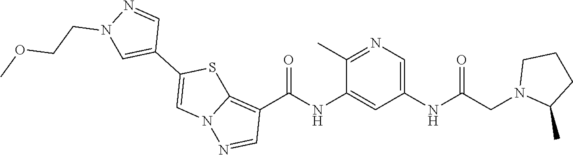 [SMILES: COCCn1cc(-c2cn3ncc(C(=O)Nc4cc(NC(=O)CN5CCC[C@H]5C)cnc4C)c3s2)cn1] | (R)-2-(1-(2-methoxyethyl)-1H- pyrazol-4-yl)-N-(2-methyl-5-(2- (2-methylpyrrolidin-1- yl)acetamido)pyridin-3- yl)pyrazolo[5,1-b]thiazole-7- carboxamide |
| 331 | 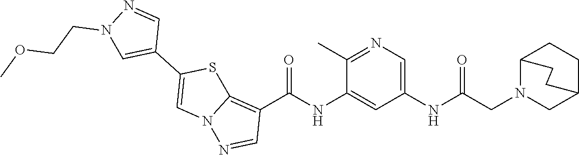 [SMILES: COCCn1cc(-c2cn3ncc(C(=O)Nc4cc(NC(=O)CN5CC6CCC5CC6)cnc4C)c3s2)cn1] | N-(5-(2-(2- azabicyclo[2.2.2]octan-2- yl)acetamido)-2-methylpyridin- 3-yl)-2-(1-(2-methoxyethyl)-1H- pyrazol-4-yl)pyrazolo[5,1- b]thiazole-7-carboxamide |
| 332 | 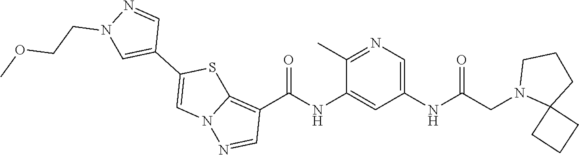 [SMILES: COCCn1cc(-c2cn3ncc(C(=O)Nc4cc(NC(=O)CN5CCCC56CCC6)cnc4C)c3s2)cn1] | N-(5-(2-(5-azaspiro[3.4]octan-5- yl)acetamido)-2-methylpyridin- 3-yl)-2-(1-(2-methoxyethyl)-1H- pyrazol-4-yl)pyrazolo[5,1- b]thiazole-7-carboxamide |
| 333 | 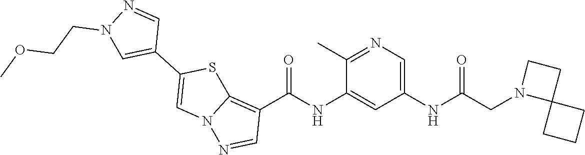 [SMILES: COCCn1cc(-c2cn3ncc(C(=O)Nc4cc(NC(=O)CN5CCC56CCC6)cnc4C)c3s2)cn1] | N-(5-(2-(1-azaspiro[3.3]heptan- 1-yl)acetamido)-2- methylpyridin-3-yl)-2-(1-(2- methoxyethyl)-1H-pyrazol-4- yl)pyrazolo[5,1-b]thiazole-7- carboxamide |
| 334 | 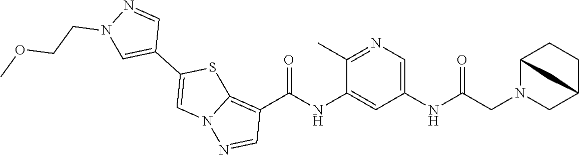 [SMILES: COCCn1cc(-c2cn3ncc(C(=O)Nc4cc(NC(=O)CN5C[C@H]6CC[C@@H]5C6)cnc4C)c3s2)cn1] | N-(5-(2-((1R,4S)-2- azabicyclo[2.2.1]heptan-2- yl)acetamido)-2-methylpyridin- 3-yl)-2-(1-(2-methoxyethyl)-1H- pyrazol-4-yl)pyrazolo[5,1- b]thiazole-7-carboxamide |
| 335 | 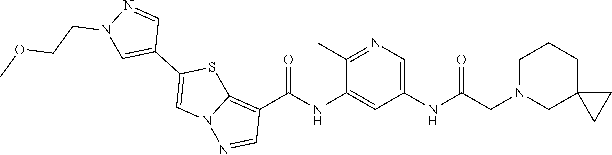 [SMILES: COCCn1cc(-c2cn3ncc(C(=O)Nc4cc(NC(=O)CN5CCCC6(CC6)C5)cnc4C)c3s2)cn1] | N-(5-(2-(5-azaspiro[2.5]octan-5- yl)acetamido)-2-methylpyridin- 3-yl)-2-(1-(2-methoxyethyl)-1H- pyrazol-4-yl)pyrazolo[5,1- b]thiazole-7-carboxamide |
| 336 | 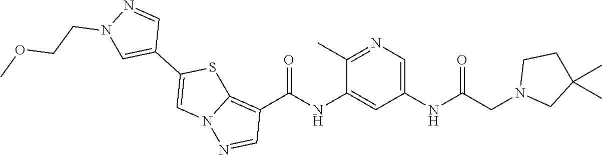 | N-(5-(2-(3,3-dimethylpyrrolidin- 1-yl)acetamido)-2- methylpyridin-3-yl)-2-(1-(2- methoxyethyl)-1H-pyrazol-4- yl)pyrazolo[5,1-b]thiazole-7- carboxamide |
| 337 | 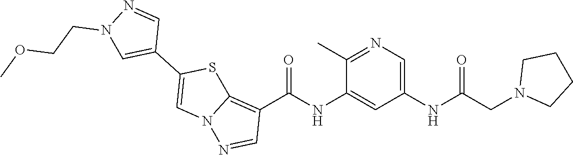 [SMILES: COCCn1cc(-c2cn3ncc(C(=O)Nc4cc(NC(=O)CN5CCCC5)cnc4C)c3s2)cn1] | 2-(1-(2-methoxyethyl)-1H- pyrazol-4-yl)-N-(2-methyl-5-(2- (pyrrolidin-1- yl)acetamido)pyridin-3- yl)pyrazolo[5,1-b]thiazole-7- carboxamide |
| 338 | 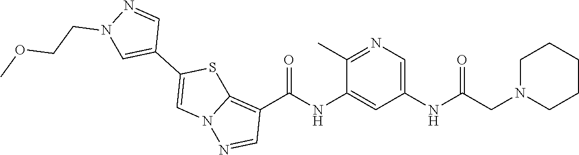 [SMILES: COCCn1cc(-c2cn3ncc(C(=O)Nc4cc(NC(=O)CN5CCCCC5)cnc4C)c3s2)cn1] | 2-(1-(2-methoxyethyl)-1H- pyrazol-4-yl)-N-(2-methyl-5-(2- (piperidin-1- yl)acetamido)pyridin-3- yl)pyrazolo[5,1-b]thiazole-7- carboxamide |
| 339 | 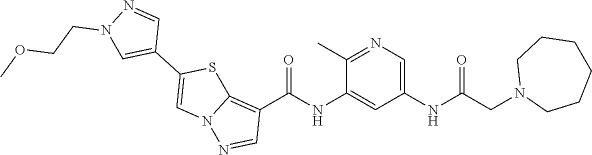 [SMILES: COCCn1cc(-c2cn3ncc(C(=O)Nc4cc(NC(=O)CN5CCCCCC5)cnc4C)c3s2)cn1] | N-(5-(2-(azepan-1- yl)acetamido)-2-methylpyridin- 3-yl)-2-(1-(2-methoxyethyl)-1H- pyrazol-4-yl)pyrazolo[5,1- b]thiazole-7-carboxamide |
| 340 | 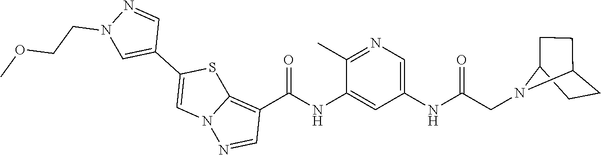 [SMILES: COCCn1cc(-c2cn3ncc(C(=O)Nc4cc(NC(=O)CN5C6CCC5CC6)cnc4C)c3s2)cn1] | N-(5-(2-(7- azabicyclo[2.2.1]heptan-7- yl)acetamido)-2-methylpyridin- 3-yl)-2-(1-(2-methoxyethyl)-1H- pyrazol-4-yl)pyrazolo[5,1- b]thiazole-7-carboxamide |
| 341 | 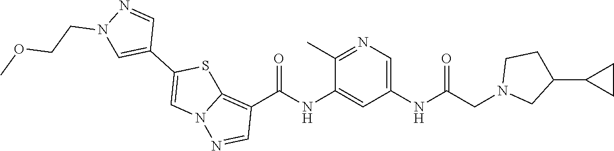 [SMILES: COCCn1cc(-c2cn3ncc(C(=O)Nc4cc(NC(=O)CN5CCC(C6CC6)C5)cnc4C)c3s2)cn1] | N-(5-(2-(3- cyclopropylpyrrolidin-1- yl)acetamido)-2-methylpyridin- 3-yl)-2-(1-(2-methoxyethyl)-1H- pyrazol-4-yl)pyrazolo[5,1- b]thiazole-7-carboxamide |
| 342 | 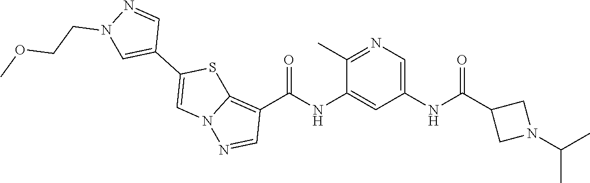 [SMILES: COCCn1cc(-c2cn3ncc(C(=O)Nc4cc(NC(=O)C5CN(C(C)C)C5)cnc4C)c3s2)cn1] | N-(5-(1-isopropylazetidine-3- carboxamido)-2-methylpyridin- 3-yl)-2-(1-(2-methoxyethyl)-1H- pyrazol-4-yl)pyrazolo[5,1- b]thiazole-7-carboxamide |
| 343 | 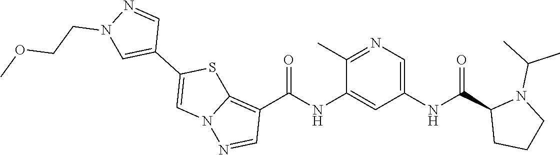 [SMILES: COCCn1cc(-c2cn3ncc(C(=O)Nc4cc(NC(=O)[C@@H]5CCCN5C(C)C)cnc4C)c3s2)cn1] | (S)-N-(5-(1- isopropylpyrrolidine-2- carboxamido)-2-methylpyridin- 3-yl)-2-(1-(2-methoxyethyl)-1H- pyrazol-4-yl)pyrazolo[5,1- b]thiazole-7-carboxamide |
| 344 | 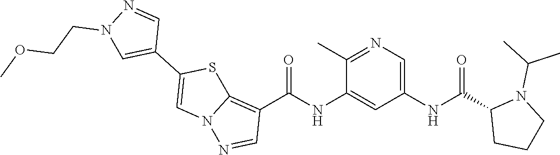 [SMILES: COCCn1cc(-c2cn3ncc(C(=O)Nc4cc(NC(=O)[C@H]5CCCN5C(C)C)cnc4C)c3s2)cn1] | (R)-N-(5-(1- isopropylpyrrolidine-2- carboxamido)-2-methylpyridin- 3-yl)-2-(1-(2-methoxyethyl)-1H- pyrazol-4-yl)pyrazolo[5,1- b]thiazole-7-carboxamide |
| 345 | 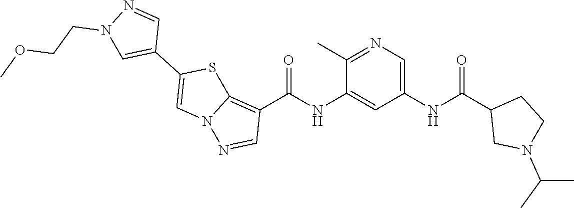 [SMILES: COCCn1cc(-c2cn3ncc(C(=O)Nc4cc(NC(=O)C5CCN(C(C)C)C5)cnc4C)c3s2)cn1] | N-(5-(1-isopropylpyrrolidine-3- carboxamido)-2-methylpyridin- 3-yl)-2-(1-(2-methoxyethyl)-1H- pyrazol-4-yl)pyrazolo[5,1- b]thiazole-7-carboxamide |
| 346 | 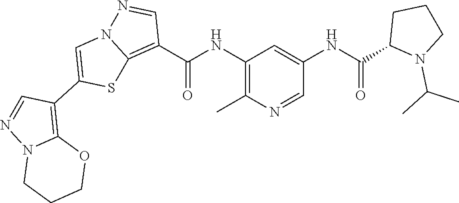 [SMILES: Cc1ncc(NC(=O)[C@@H]2CCCN2C(C)C)cc1NC(=O)c1cnn2cc(-c3cnn4c3OCCC4)sc12] | (S)-2-(6,7-dihydro-5H- pyrazolo[5,1-b][1,3]oxazin-3- yl)-N-(5-(1- isopropylpyrrolidine-2- carboxamido)-2-methylpyridin- 3-yl)pyrazolo[5,1-b]thiazole-7- carboxamide |
| 347 | 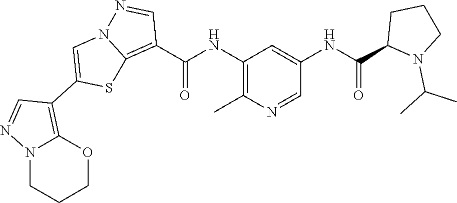 [SMILES: Cc1ncc(NC(=O)[C@H]2CCCN2C(C)C)cc1NC(=O)c1cnn2cc(-c3cnn4c3OCCC4)sc12] | (R)-2-(6,7-dihydro-5H- pyrazolo[5,1-b][1,3]oxazin-3- yl)-N-(5-(1- isopropylpyrrolidine-2- carboxamido)-2-methylpyridin- 3-yl)pyrazolo[5,1-b]thiazole-7- carboxamide |
| 348 | 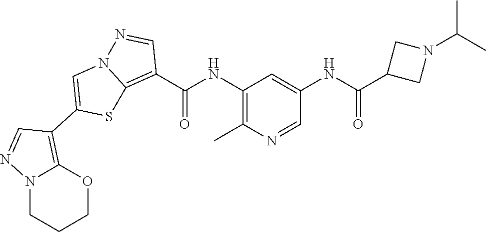 [SMILES: Cc1ncc(NC(=O)C2CN(C(C)C)C2)cc1NC(=O)c1cnn2cc(-c3cnn4c3OCCC4)sc12] | 2-(6,7-dihydro-5H-pyrazolo[5,1- b][1,3]oxazin-3-yl)-N-(5-(1- isopropylazetidine-3- carboxamido)-2-methylpyridin- 3-yl)pyrazolo[5,1-b]thiazole-7- carboxamide |
| 349 | 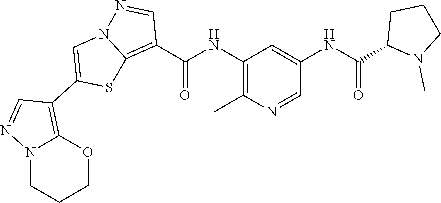 [SMILES: Cc1ncc(NC(=O)[C@@H]2CCCN2C)cc1NC(=O)c1cnn2cc(-c3cnn4c3OCCC4)sc12] | (S)-2-(6,7-dihydro-5H- pyrazolo[5,1-b][1,3]oxazin-3- yl)-N-(2-methyl-5-(1- methylpyrrolidine-2- carboxamido)pyridin-3- yl)pyrazolo[5,1-b]thiazole-7- carboxamide |
| 350 | 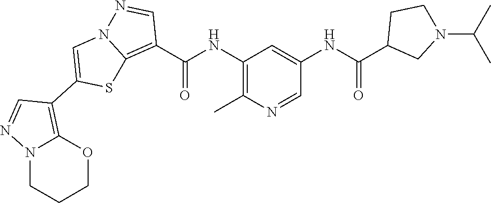 [SMILES: Cc1ncc(NC(=O)C2CCN(C(C)C)C2)cc1NC(=O)c1cnn2cc(-c3cnn4c3OCCC4)sc12] | 2-(6,7-dihydro-5H-pyrazolo[5,1- b][1,3]oxazin-3-yl)-N-(5-(1- isopropylpyrrolidine-3- carboxamido)-2-methylpyridin- 3-yl)pyrazolo[5,1-b]thiazole-7- carboxamide |
| 351 | 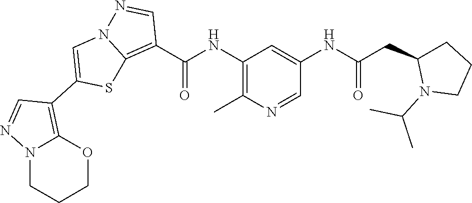 [SMILES: Cc1ncc(NC(=O)C[C@H]2CCCN2C(C)C)cc1NC(=O)c1cnn2cc(-c3cnn4c3OCCC4)sc12] | (R)-2-(6,7-dihydro-5H- pyrazolo[5,1-b/[1,3]oxazin-3- yl)-N-(5-(2-(1- isopropylpyrrolidin-2- yl)acetamido)-2-methylpyridin- 3-yl)pyrazolo[5,1-b]thiazole-7- carboxamide |
| 352 | 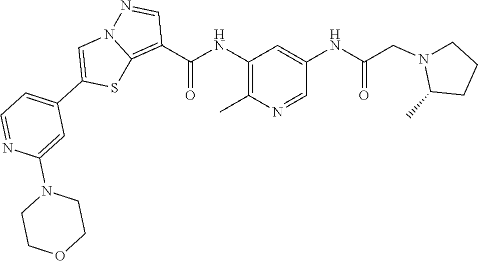 [SMILES: Cc1ncc(NC(=O)CN2CCC[C@@H]2C)cc1NC(=O)c1cnn2cc(-c3ccnc(N4CCOCC4)c3)sc12] | (S)-N-(2-methyl-5-(2-(2- methylpyrrolidin-1- yl)acetamido)pyridin-3-yl)-2-(2- morpholinopyridin-4- yl)pyrazolo[5,1-b]thiazole-7- carboxamide |
| 353 | 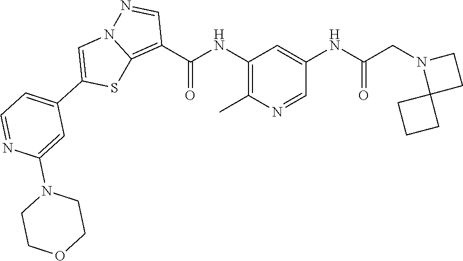 [SMILES: Cc1ncc(NC(=O)CN2CCC23CCC3)cc1NC(=O)c1cnn2cc(-c3ccnc(N4CCOCC4)c3)sc12] | N-(5-(2-(1-azaspiro[3.3]heptan- 1-yl)acetamido)-2- methylpyridin-3-yl)-2-(2- morpholinopyridin-4- yl)pyrazolo[5,1-b]thiazole-7- carboxamide |
| 354 | 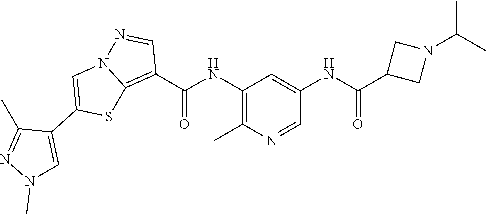 [SMILES: Cc1ncc(NC(=O)C2CN(C(C)C)C2)cc1NC(=O)c1cnn2cc(-c3cn(C)nc3C)sc12] | 2-(1,3-dimethyl-1H-pyrazol-4- yl)-N-(5-(1-isopropylazetidine- 3-carboxamido)-2- methylpyridin-3- yl)pyrazolo[5,1-b]thiazole-7- carboxamide |
| 355 | 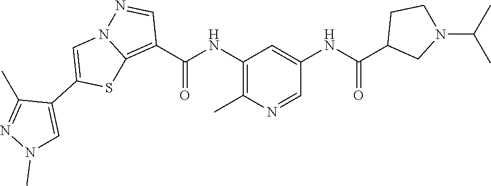 [SMILES: Cc1ncc(NC(=O)C2CCN(C(C)C)C2)cc1NC(=O)c1cnn2cc(-c3cn(C)nc3C)sc12] | 2-(1,3-dimethyl-1H-pyrazol-4- yl)-N-(5-(1- isopropylpyrrolidine-3- carboxamido)-2-methylpyridin- 3-yl)pyrazolo[5,1-b]thiazole-7- carboxamide |
| 356 | 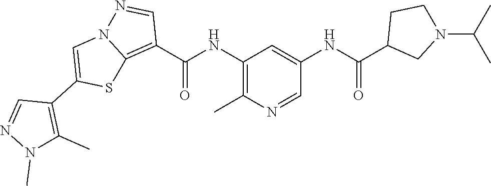 [SMILES: Cc1ncc(NC(=O)C2CCN(C(C)C)C2)cc1NC(=O)c1cnn2cc(-c3cnn(C)c3C)sc12] | 2-(1,5-dimethyl-1H-pyrazol-4- yl)-N-(5-(1- isopropylpyrrolidine-3- carboxamido)-2-methylpyridin- 3-yl)pyrazolo[5,1-b]thiazole-7- carboxamide |
| 357 | 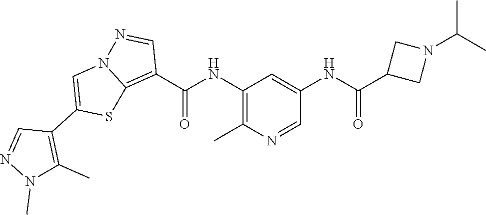 [SMILES: Cc1ncc(NC(=O)C2CN(C(C)C)C2)cc1NC(=O)c1cnn2cc(-c3cnn(C)c3C)sc12] | 2-(1,5-dimethyl-1H-pyrazol-4- yl)-N-(5-(1-isopropylazetidine- 3-carboxamido)-2- methylpyridin-3- yl)pyrazolo[5,1-b]thiazole-7- carboxamide |
| 358 | 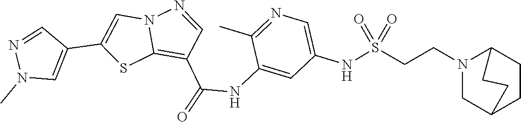 [SMILES: Cc1ncc(NS(=O)(=O)CCN2CC3CCC2CC3)cc1NC(=O)c1cnn2cc(-c3cnn(C)c3)sc12] | N-(5-((2-(2- azabicyclo[2.2.2]octan-2- yl)ethyl)sulfonamido)-2- methylpyridin-3-yl)-2-(1- methyl-1H-pyrazol-4- yl)pyrazolo[5,1-b]thiazole-7- carboxamide |
| 359 | 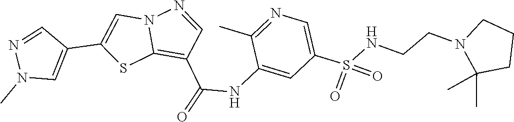 [SMILES: Cc1ncc(S(=O)(=O)NCCN2CCCC2(C)C)cc1NC(=O)c1cnn2cc(-c3cnn(C)c3)sc12] | N-(5-(N-(2-(2,2- dimethylpyrrolidin-1- yl)ethyl)sulfamoyl)-2- methylpyridin-3-yl)-2-(1- methyl-1H-pyrazol-4- yl)pyrazolo[5,1-b]thiazole-7- carboxamide |
| 360 | 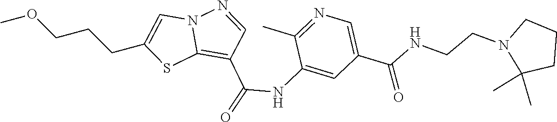 [SMILES: COCCCc1cn2ncc(C(=O)Nc3cc(C(=O)NCCN4CCCC4(C)C)cnc3C)c2s1] | N-(5-((2-(2,2- dimethylpyrrolidin-1- yl)ethyl)carbamoyl)-2- methylpyridin-3-yl)-2-(3- methoxypropyl)pyrazolo[5,1- b]thiazole-7-carboxamide |
| 361 | 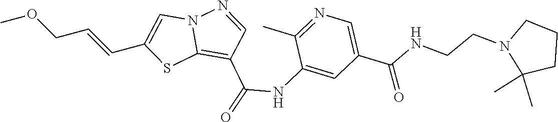 [SMILES: COC/C=C/c1cn2ncc(C(=O)Nc3cc(C(=O)NCCN4CCCC4(C)C)cnc3C)c2s1] | (E)-N-(5-((2-(2,2- dimethylpyrrolidin-1- yl)ethyl)carbamoyl)-2- methylpyridin-3-yl)-2-(3- methoxyprop-1-en-1- yl)pyrazolo[5,1-b]thiazole-7- carboxamide |
| 362 | 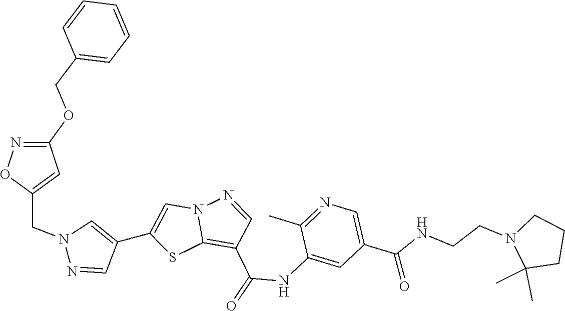 [SMILES: Cc1ncc(C(=O)NCCN2CCCC2(C)C)cc1NC(=O)c1cnn2cc(-c3cnn(Cc4cc(OCc5ccccc5)no4)c3)sc12] | 2-(1-((3-(benzyloxy)isoxazol-5- yl)methyl)-1H-pyrazol-4-yl)-N- (5-((2-(2,2-dimethylpyrrolidin-1- yl)ethyl)carbamoyl)-2- methylpyridin-3- yl)pyrazolo[5,1-b]thiazole-7- carboxamide |
| 363 | 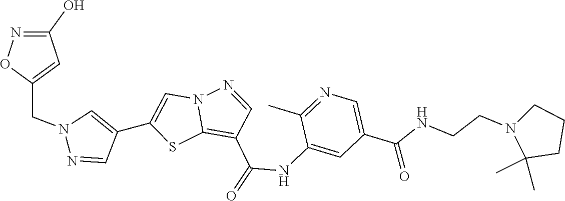 [SMILES: Cc1ncc(C(=O)NCCN2CCCC2(C)C)cc1NC(=O)c1cnn2cc(-c3cnn(Cc4cc(O)no4)c3)sc12] | N-(5-((2-(2,2- dimethylpyrrolidin-1- yl)ethyl)carbamoyl)-2- methylpyridin-3-yl)-2-(1-((3- hydroxyisoxazol-5-yl)methyl)- 1H-pyrazol-4-yl)pyrazolo[5,1- b]thiazole-7-carboxamide |
| 364 | 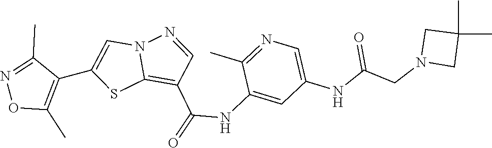 [SMILES: Cc1ncc(NC(=O)CN2CC(C)(C)C2)cc1NC(=O)c1cnn2cc(-c3c(C)noc3C)sc12] | N-(5-(2-(3,3-dimethylazetidin-1- yl)acetamido)-2-methylpyridin- 3-yl)-2-(3,5-dimethylisoxazol-4- yl)pyrazolo[5,1-b]thiazole-7- carboxamide |
| 365 | 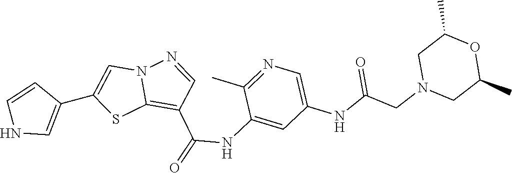 [SMILES: Cc1ncc(NC(=O)CN2C[C@H](C)O[C@@H](C)C2)cc1NC(=O)c1cnn2cc(-c3cc[nH]c3)sc12] | N-(5-(2-(2,6-trans- dimethylmorpholino)acetamido)- 2-methylpyridin-3-yl)-2-(1H- pyrrol-3-yl)pyrazolo[5,1- b]thiazole-7-carboxamide |
| 366 | 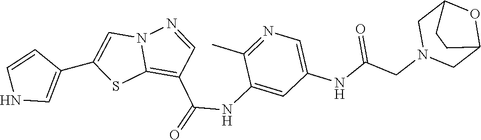 [SMILES: Cc1ncc(NC(=O)CN2CC3CCC(C2)O3)cc1NC(=O)c1cnn2cc(-c3cc[nH]c3)sc12] | N-(5-(2-(8-oxa-3- azabicyclo[3.2.1]octan-3- yl)acetamido)-2-methylpyridin- 3-yl)-2-(1H-pyrrol-3- yl)pyrazolo[5,1-b]thiazole-7- carboxamide |
| 367 | 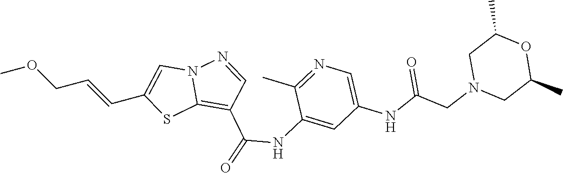 | N-(5-(2-(trans-2,6- dimethylmorpholino)acetamido)- 2-methylpyridin-3-yl)-2-((E)-3- methoxyprop-1-en-1- yl)pyrazolo[5,1-b]thiazole-7- carboxamide |
| 368 | 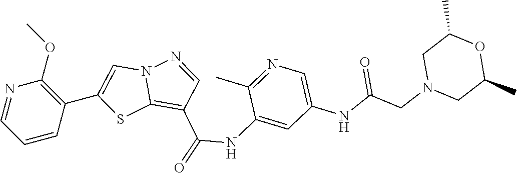 [SMILES: COc1ncccc1-c1cn2ncc(C(=O)Nc3cc(NC(=O)CN4C[C@H](C)O[C@@H](C)C4)cnc3C)c2s1] | N-(5-(2-(trans-2,6- dimethylmorpholino)acetamido)- 2-methylpyridin-3-yl)-2-(2- methoxypyridin-3- yl)pyrazolo[5,1-b]thiazole-7- carboxamide |
| 369 | 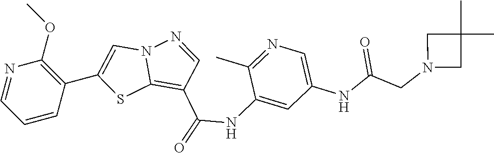 [SMILES: COc1ncccc1-c1cn2ncc(C(=O)Nc3cc(NC(=O)CN4CC(C)(C)C4)cnc3C)c2s1] | N-(5-(2-(3,3-dimethylazetidin-1- yl)acetamido)-2-methylpyridin- 3-yl)-2-(2-methoxypyridin-3- yl)pyrazolo[5,1-b]thiazole-7- carboxamide |
| 370 | 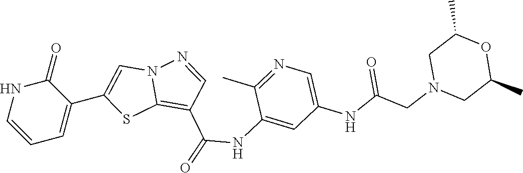 [SMILES: Cc1ncc(NC(=O)CN2C[C@H](C)O[C@@H](C)C2)cc1NC(=O)c1cnn2cc(-c3ccc[nH]c3=O)sc12] | N-(5-(2-(trans-2,6- dimethylmorpholino)acetamido)- 2-methylpyridin-3-yl)-2-(2-oxo- 1,2-dihydropyridin-3- yl)pyrazolo[5,1-b]thiazole-7- carboxamide |
| 371 | 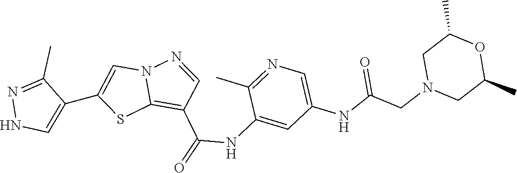 [SMILES: Cc1ncc(NC(=O)CN2C[C@H](C)O[C@@H](C)C2)cc1NC(=O)c1cnn2cc(-c3c[nH]nc3C)sc12] | N-(5-(2-(trans-2,6- dimethylmorpholino)acetamido)- 2-methylpyridin-3-yl)-2-(3- methyl-1H-pyrazol-4- yl)pyrazolo[5,1-b]thiazole-7- carboxamide |
| 372 | 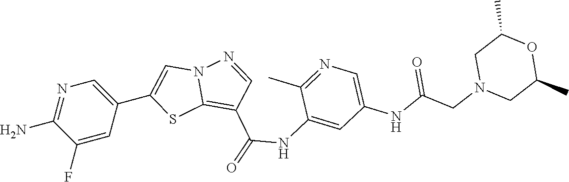 [SMILES: Cc1ncc(NC(=O)CN2C[C@H](C)O[C@@H](C)C2)cc1NC(=O)c1cnn2cc(-c3cnc(N)c(F)c3)sc12] | 2-(6-amino-5-fluoropyridin-3- yl)-N-(5-(2-(trans-2,6- dimethylmorpholino)acetamido)- 2-methylpyridin-3- yl)pyrazolo[5,1-b]thiazole-7- carboxamide |
| 373 | 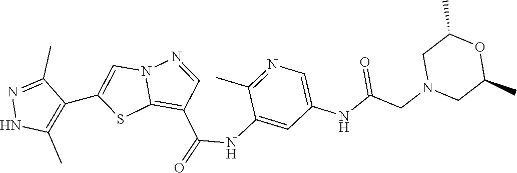 [SMILES: Cc1ncc(NC(=O)CN2C[C@H](C)O[C@@H](C)C2)cc1NC(=O)c1cnn2cc(-c3c(C)n[nH]c3C)sc12] | 2-(3,5-dimethyl-1H-pyrazol-4- yl)-N-(5-(2-(trans-2,6- dimethylmorpholino)acetamido)- 2-methylpyridin-3- yl)pyrazolo[5,1-b]thiazole-7- carboxamide |
| 374 | 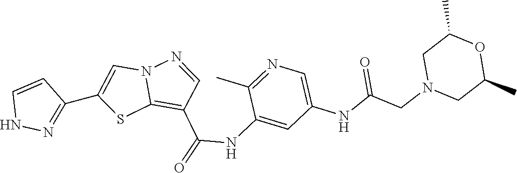 [SMILES: Cc1ncc(NC(=O)CN2C[C@H](C)O[C@@H](C)C2)cc1NC(=O)c1cnn2cc(-c3cc[nH]n3)sc12] | N-(5-(2-(trans-2,6- dimethylmorpholino)acetamido)- 2-methylpyridin-3-yl)-2-(1H- pyrazol-3-yl)pyrazolo[5,1- b]thiazole-7-carboxamide |
| 375 | 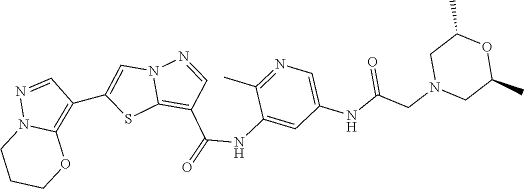 [SMILES: Cc1ncc(NC(=O)CN2C[C@H](C)O[C@@H](C)C2)cc1NC(=O)c1cnn2cc(-c3cnn4c3OCCC4)sc12] | 2-(6,7-dihydro-5H-pyrazolo[5,1- b][1,3]oxazin-3-yl)-N-(5-(2- (trans-2,6- dimethylmorpholino)acetamido)- 2-methylpyridin-3- yl)pyrazolo[5,1-b]thiazole-7- carboxamide |
| 376 | 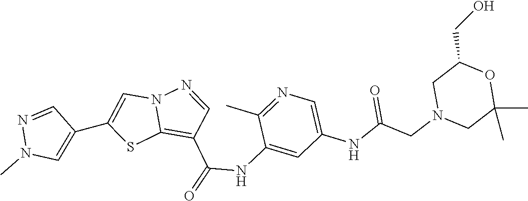 | (R)-N-(5-(2-(6-(hydroxymethyl)- 2,2- dimethylmorpholino)acetamido)- 2-methylpyridin-3-yl)-2-(1- methyl-1H-pyrazol-4- yl)pyrazolo[5,1-b]thiazole-7- carboxamide |
| 377 | 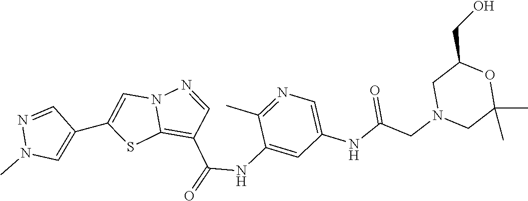 | (S)-N-(5-(2-(6-(hydroxymethyl)- 2,2- dimethylmorpholino)acetamido)- 2-methylpyridin-3-yl)-2-(1- methyl-1H-pyrazol-4- yl)pyrazolo[5,1-b]thiazole-7- carboxamide |
| 378 | 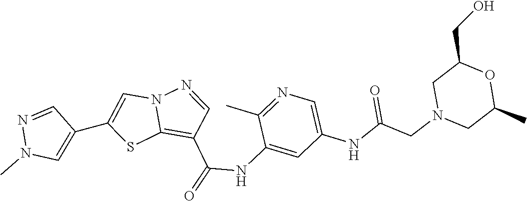 | N-(5-(2-(cis-2-(hydroxymethyl)- 6- methylmorpholino)acetamido)- 2-methylpyridin-3-yl)-2-(1- methyl-1H-pyrazol-4- yl)pyrazolo[5,1-b]thiazole-7- carboxamide |
| 379 | 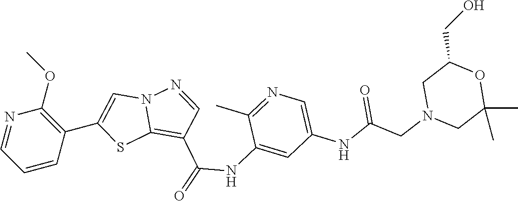 [SMILES: COc1ncccc1-c1cn2ncc(C(=O)Nc3cc(NC(=O)CN4C[C@H](CO)OC(C)(C)C4)cnc3C)c2s1] | (R)-N-(5-(2-(6-(hydroxymethyl)- 2,2- dimethylmorpholino)acetamido)- 2-methylpyridin-3-yl)-2-(2- methoxypyridin-3- yl)pyrazolo[5,1-b]thiazole-7- carboxamide |
| 380 | 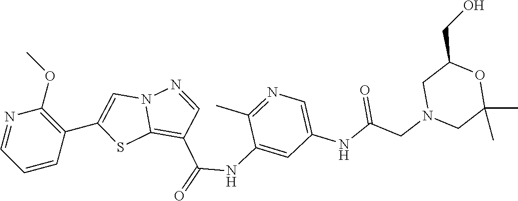 [SMILES: COc1ncccc1-c1cn2ncc(C(=O)Nc3cc(NC(=O)CN4C[C@@H](CO)OC(C)(C)C4)cnc3C)c2s1] | (S)-N-(5-(2-(6-(hydroxymethyl)- 2,2- dimethylmorpholino)acetamido)- 2-methylpyridin-3-yl)-2-(2- methoxypyridin-3- yl)pyrazolo[5,1-b]thiazole-7- carboxamide |
| 381 | 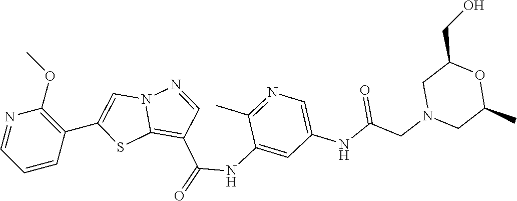 [SMILES: COc1ncccc1-c1cn2ncc(C(=O)Nc3cc(NC(=O)CN4C[C@@H](CO)O[C@@H](C)C4)cnc3C)c2s1] | N-(5-(2-(cis-2-(hydroxymethyl)- 6- methylmorpholino)acetamido)- 2-methylpyridin-3-yl)-2-(2- methoxypyridin-3- yl)pyrazolo[5,1-b]thiazole-7- carboxamide |
| 382 | 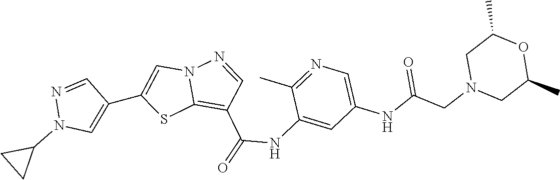 [SMILES: Cc1ncc(NC(=O)CN2C[C@H](C)O[C@@H](C)C2)cc1NC(=O)c1cnn2cc(-c3cnn(C4CC4)c3)sc12] | 2-(1-cyclopropyl-1H-pyrazol-4- yl)-N-(5-(2-(trans-2,6- dimethylmorpholino)acetamido)- 2-methylpyridin-3- yl)pyrazolo[5,1-b]thiazole-7- carboxamide |
| 383 | 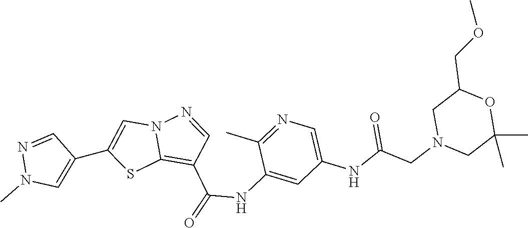 | N-(5-(2-(6-(methoxymethyl)- 2,2- dimethylmorpholino)acetamido)- 2-methylpyridin-3-yl)-2-(1- methyl-1H-pyrazol-4- yl)pyrazolo[5,1-b]thiazole-7- carboxamide |
| 384 | 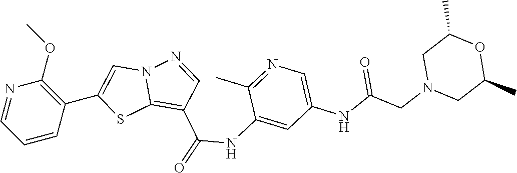 [SMILES: COc1ncccc1-c1cn2ncc(C(=O)Nc3cc(NC(=O)CN4C[C@H](C)O[C@@H](C)C4)cnc3C)c2s1] | N-(5-(2-(trans-2,6- dimethylmorpholino)acetamido)- 2-methylpyridin-3-yl)-2-(2- methoxypyridin-3- yl)pyrazolo[5,1-b]thiazole-7- carboxamide |
| 385 | 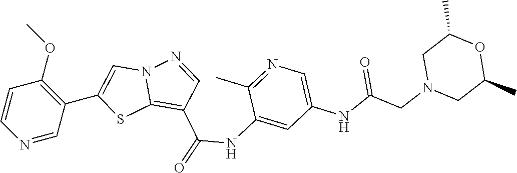 [SMILES: COc1ccncc1-c1cn2ncc(C(=O)Nc3cc(NC(=O)CN4C[C@H](C)O[C@@H](C)C4)cnc3C)c2s1] | N-(5-(2-(trans-2,6- dimethylmorpholino)acetamido)- 2-methylpyridin-3-yl)-2-(4- methoxypyridin-3- yl)pyrazolo[5,1-b]thiazole-7- carboxamide |
| 386 | 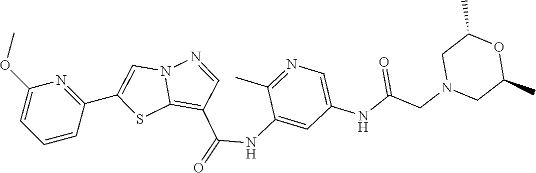 [SMILES: COc1cccc(-c2cn3ncc(C(=O)Nc4cc(NC(=O)CN5C[C@H](C)O[C@@H](C)C5)cnc4C)c3s2)n1] | N-(5-(2-(trans-2,6- dimethylmorpholino)acetamido)- 2-methylpyridin-3-yl)-2-(6- methoxypyridin-2- yl)pyrazolo[5,1-b]thiazole-7- carboxamide |
| 387 | 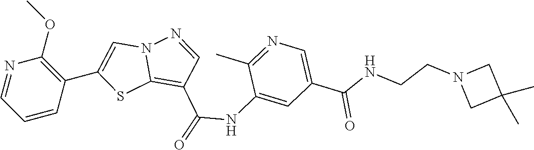 [SMILES: COc1ncccc1-c1cn2ncc(C(=O)Nc3cc(C(=O)NCCN4CC(C)(C)C4)cnc3C)c2s1] | N-(5-((2-(3,3-dimethylazetidin- 1-yl)ethyl)carbamoyl)-2- methylpyridin-3-yl)-2-(2- methoxypyridin-3- yl)pyrazolo[5,1-b]thiazole-7- carboxamide |
| 388 | 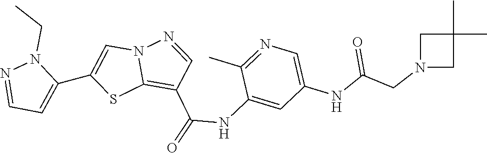 [SMILES: CCn1nccc1-c1cn2ncc(C(=O)Nc3cc(NC(=O)CN4CC(C)(C)C4)cnc3C)c2s1] | N-(5-(2-(3,3-dimethylazetidin-1- yl)acetamido)-2-methylpyridin- 3-yl)-2-(1-ethyl-1H-pyrazol-5- yl)pyrazolo[5,1-b]thiazole-7- carboxamide |
| 389 | 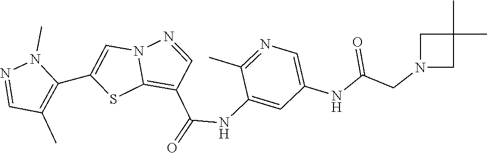 [SMILES: Cc1cnn(C)c1-c1cn2ncc(C(=O)Nc3cc(NC(=O)CN4CC(C)(C)C4)cnc3C)c2s1] | 2-(1,4-dimethyl-1H-pyrazol-5- yl)-N-(5-(2-(3,3- dimethylazetidin-1- yl)acetamido)-2-methylpyridin- 3-yl)pyrazolo[5,1-b]thiazole-7- carboxamide |
| 390 | 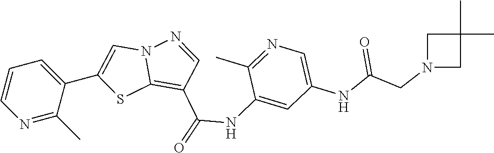 [SMILES: Cc1ncc(NC(=O)CN2CC(C)(C)C2)cc1NC(=O)c1cnn2cc(-c3cccnc3C)sc12] | N-(5-(2-(3,3-dimethylazetidin-1- yl)acetamido)-2-methylpyridin- 3-yl)-2-(2-methylpyridin-3- yl)pyrazolo[5,1-b]thiazole-7- carboxamide |
| 391 | 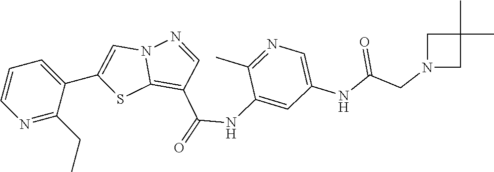 [SMILES: CCc1ncccc1-c1cn2ncc(C(=O)Nc3cc(NC(=O)CN4CC(C)(C)C4)cnc3C)c2s1] | N-(5-(2-(3,3-dimethylazetidin-1- yl)acetamido)-2-methylpyridin- 3-yl)-2-(2-ethylpyridin-3- yl)pyrazolo[5,1-b]thiazole-7- carboxamide |
| 392 | 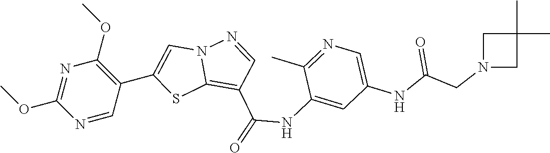 | 2-(2,4-dimethoxypyrimidin-5- yl)-N-(5-(2-(3,3- dimethylazetidin-1- yl)acetamido)-2-methylpyridin- 3-yl)pyrazolo[5,1-b]thiazole-7- carboxamide |
| 393 | 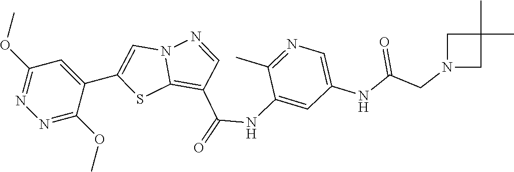 [SMILES: COc1cc(-c2cn3ncc(C(=O)Nc4cc(NC(=O)CN5CC(C)(C)C5)cnc4C)c3s2)c(OC)nn1] | 2-(3,6-dimethoxypyridazin-4- yl)-N-(5-(2-(3,3- dimethylazetidin-1- yl)acetamido)-2-methylpyridin- 3-yl)pyrazolo[5,1-b]thiazole-7- carboxamide |
| 394 | 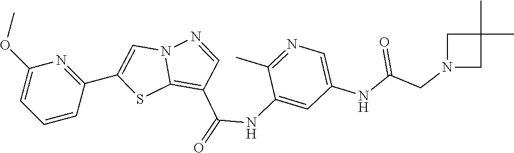 [SMILES: COc1cccc(-c2cn3ncc(C(=O)Nc4cc(NC(=O)CN5CC(C)(C)C5)cnc4C)c3s2)n1] | N-(5-(2-(3,3-dimethylazetidin-1- yl)acetamido)-2-methylpyridin- 3-yl)-2-(6-methoxypyridin-2- yl)pyrazolo[5,1-b]thiazole-7- carboxamide |
| 395 | 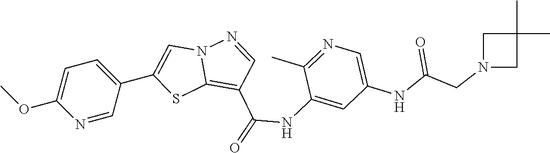 | N-(5-(2-(3,3-dimethylazetidin-1- yl)acetamido)-2-methylpyridin- 3-yl)-2-(6-methoxypyridin-3- yl)pyrazolo[5,1-b]thiazole-7- carboxamide |
| 396 | 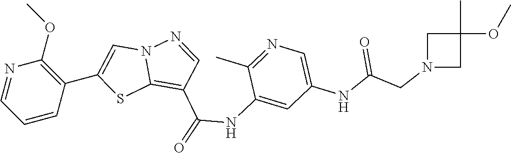 [SMILES: COc1ncccc1-c1cn2ncc(C(=O)Nc3cc(NC(=O)CN4CC(C)(OC)C4)cnc3C)c2s1] | N-(5-(2-(3-methoxy-3- methylazetidin-1-yl)acetamido)- 2-methylpyridin-3-yl)-2-(2- methoxypyridin-3- yl)pyrazolo[5,1-b]thiazole-7- carboxamide |
| 397 | 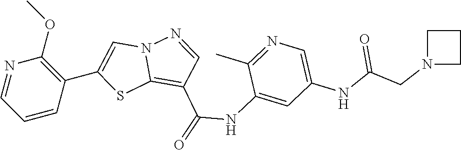 [SMILES: COc1ncccc1-c1cn2ncc(C(=O)Nc3cc(NC(=O)CN4CCC4)cnc3C)c2s1] | N-(5-(2-(azetidin-1- yl)acetamido)-2-methylpyridin- 3-yl)-2-(2-methoxypyridin-3- yl)pyrazolo[5,1-b]thiazole-7- carboxamide |
| 398 | 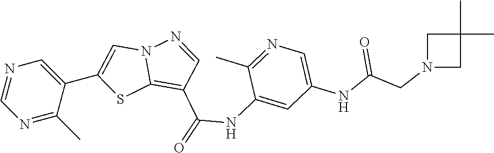 [SMILES: Cc1ncc(NC(=O)CN2CC(C)(C)C2)cc1NC(=O)c1cnn2cc(-c3cncnc3C)sc12] | N-(5-(2-(3,3-dimethylazetidin-1- yl)acetamido)-2-methylpyridin- 3-yl)-2-(4-methylpyrimidin-5- yl)pyrazolo[5,1-b]thiazole-7- carboxamide |
| 399 | 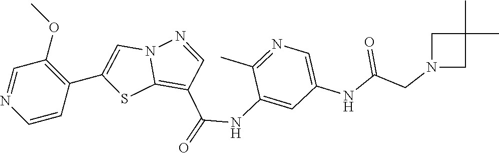 [SMILES: COc1cnccc1-c1cn2ncc(C(=O)Nc3cc(NC(=O)CN4CC(C)(C)C4)cnc3C)c2s1] | N-(5-(2-(3,3-dimethylazetidin-1- yl)acetamido)-2-methylpyridin- 3-yl)-2-(3-methoxypyridin-4- yl)pyrazolo[5,1-b]thiazole-7- carboxamide |
| 400 | 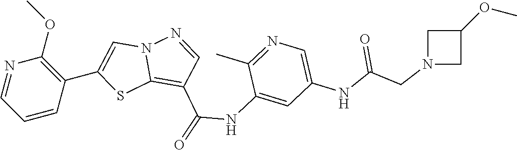 [SMILES: COc1ncccc1-c1cn2ncc(C(=O)Nc3cc(NC(=O)CN4CC(OC)C4)cnc3C)c2s1] | N-(5-(2-(3-methoxyazetidin-1- yl)acetamido)-2-methylpyridin- 3-yl)-2-(2-methoxypyridin-3- yl)pyrazolo[5,1-b]thiazole-7- carboxamide |
| 401 | 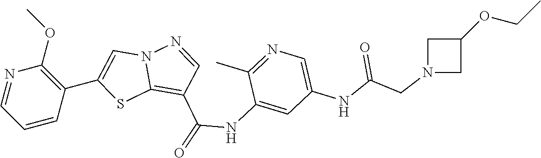 [SMILES: CCOC1CN(CC(=O)Nc2cnc(C)c(NC(=O)c3cnn4cc(-c5cccnc5OC)sc34)c2)C1] | N-(5-(2-(3-methoxyazetidin-1- yl)acetamido)-2-methylpyridin- 3-yl)-2-(2-ethoxypyridin-3- yl)pyrazolo[5,1-b]thiazole-7- carboxamide |
| 402 | 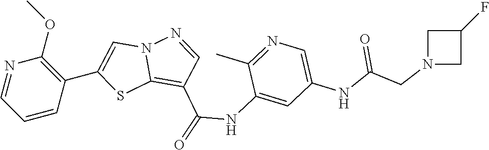 [SMILES: COc1ncccc1-c1cn2ncc(C(=O)Nc3cc(NC(=O)CN4CC(F)C4)cnc3C)c2s1] | N-(5-(2-(3-fluoroazetidin-1- yl)acetamido)-2-methylpyridin- 3-yl)-2-(2-methoxypyridin-3- yl)pyrazolo[5,1-b]thiazole-7- carboxamide |
| 403 | 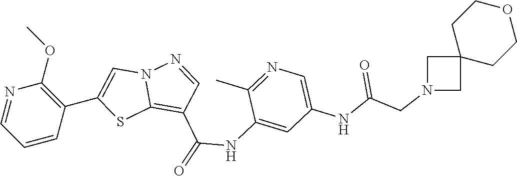 [SMILES: COc1ncccc1-c1cn2ncc(C(=O)Nc3cc(NC(=O)CN4CC5(CCOCC5)C4)cnc3C)c2s1] | N-(5-(2-(7-oxa-2- azaspiro[3.5]nonan-2- yl)acetamido)-2-methylpyridin- 3-yl)-2-(2-methoxypyridin-3- yl)pyrazolo[5,1-b]thiazole-7- carboxamide carboxamide |
| 404 | 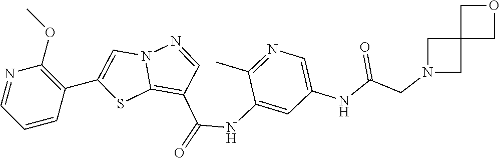 [SMILES: COc1ncccc1-c1cn2ncc(C(=O)Nc3cc(NC(=O)CN4CC5(COC5)C4)cnc3C)c2s1] | N-(5-(2-(2-oxa-6- azaspiro[3.3]heptan-6- yl)acetamido)-2-methylpyridin- 3-yl)-2-(2-methoxypyridin-3- yl)pyrazolo[5,1-b]thiazole-7- carboxamide |
| 405 | 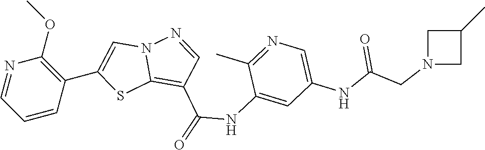 [SMILES: COc1ncccc1-c1cn2ncc(C(=O)Nc3cc(NC(=O)CN4CC(C)C4)cnc3C)c2s1] | 2-(2-methoxypyridin-3-yl)-N-(2- methyl-5-(2-(3-methylazetidin- 1-yl)acetamido)pyridin-3- yl)pyrazolo[5,1-b]thiazole-7- carboxamide |
| 406 | 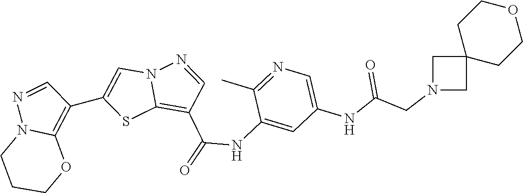 [SMILES: Cc1ncc(NC(=O)CN2CC3(CCOCC3)C2)cc1NC(=O)c1cnn2cc(-c3cnn4c3OCCC4)sc12] | N-(5-(2-(7-oxa-2- azaspiro[3.5]nonan-2- yl)acetamido)-2-methylpyridin- 3-yl)-2-(6,7-dihydro-5H- pyrazolo[5,1-b][1,3]oxazin-3- yl)pyrazolo[5,1-b]thiazole-7- carboxamide |
| 407 | 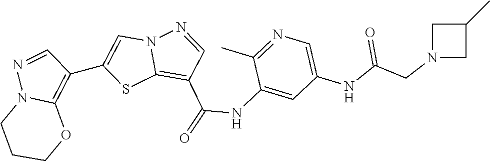 [SMILES: Cc1ncc(NC(=O)CN2CC(C)C2)cc1NC(=O)c1cnn2cc(-c3cnn4c3OCCC4)sc12] | 2-(6,7-dihydro-5H-pyrazolo[5,1- b][1,3]oxazin-3-yl)-N-(2-methyl- 5-(2-(3-methylazetidin-1- yl)acetamido)pyridin-3- yl)pyrazolo[5,1-b]thiazole-7- carboxamide |
| 408 | 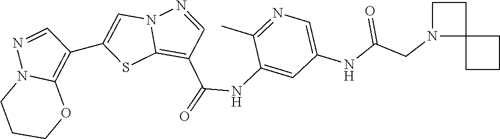 [SMILES: Cc1ncc(NC(=O)CN2CCC23CCC3)cc1NC(=O)c1cnn2cc(-c3cnn4c3OCCC4)sc12] | N-(5-(2-(1-azaspiro[3.3]heptan- 1-yl)acetamido)-2- methylpyridin-3-yl)-2-(6,7- dihydro-5H-pyrazolo[5,1- b][1,3]oxazin-3-yl)pyrazolo[5,1- b]thiazole-7-carboxamide |
| 409 | 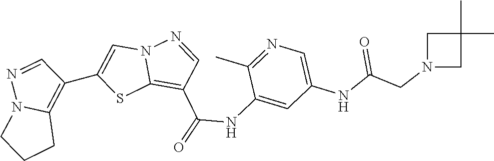 [SMILES: Cc1ncc(NC(=O)CN2CC(C)(C)C2)cc1NC(=O)c1cnn2cc(-c3cnn4c3CCC4)sc12] | 2-(5,6-dihydro-4H-pyrrolo[1,2- b]pyrazol-3-yl)-N-(5-(2-(3,3- dimethylazetidin-1- yl)acetamido)-2-methylpyridin- 3-yl)pyrazolo[5,1-b]thiazole-7- carboxamide |
| 410 | 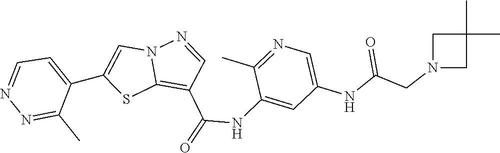 [SMILES: Cc1ncc(NC(=O)CN2CC(C)(C)C2)cc1NC(=O)c1cnn2cc(-c3ccnnc3C)sc12] | N-(5-(2-(3,3-dimethylazetidin-1- yl)acetamido)-2-methylpyridin- 3-yl)-2-(3-methylpyridazin-4- yl)pyrazolo[5,1-b]thiazole-7- carboxamide |
| 411 | 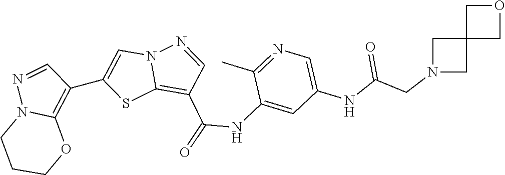 [SMILES: Cc1ncc(NC(=O)CN2CC3(COC3)C2)cc1NC(=O)c1cnn2cc(-c3cnn4c3OCCC4)sc12] | N-(5-(2-(2-oxa-6- azaspiro[3.3]heptan-6- yl)acetamido)-2-methylpyridin- 3-yl)-2-(6,7-dihydro-5H- pyrazolo[5,1-b][1,3]oxazin-3- yl)pyrazolo[5,1-b]thiazole-7- carboxamide |
| 412 | 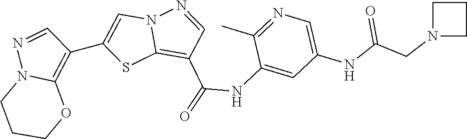 [SMILES: Cc1ncc(NC(=O)CN2CCC2)cc1NC(=O)c1cnn2cc(-c3cnn4c3OCCC4)sc12] | N-(5-(2-(azetidin-1- yl)acetamido)-2-methylpyridin- 3-yl)-2-(6,7-dihydro-5H- pyrazolo[5,1-b][1,3]oxazin-3- yl)pyrazolo[5,1-b]thiazole-7- carboxamide |
| 413 | 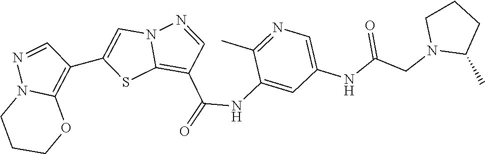 [SMILES: Cc1ncc(NC(=O)CN2CCC[C@@H]2C)cc1NC(=O)c1cnn2cc(-c3cnn4c3OCCC4)sc12] | (S)-2-(6,7-dihydro-5H- pyrazolo[5,1-b][1,3]oxazin-3- yl)-N-(2-methyl-5-(2-(2- methylpyrrolidin-1- yl)acetamido)pyridin-3- yl)pyrazolo[5,1-b]thiazole-7- carboxamide |
| 414 | 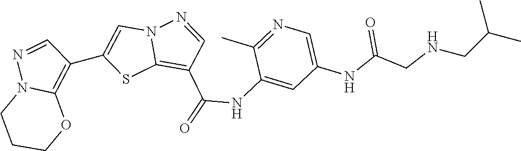 [SMILES: Cc1ncc(NC(=O)CNCC(C)C)cc1NC(=O)c1cnn2cc(-c3cnn4c3OCCC4)sc12] | 2-(6,7-dihydro-5H-pyrazolo[5,1- b][1,3]oxazin-3-yl)-N-(5-(2- (isobutylamino)acetamido)-2- methylpyridin-3- yl)pyrazolo[5,1-b]thiazole-7- carboxamide |
| 415 | 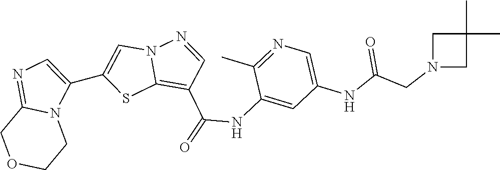 [SMILES: Cc1ncc(NC(=O)CN2CC(C)(C)C2)cc1NC(=O)c1cnn2cc(-c3cnc4n3CCOC4)sc12] | 2-(5,6-dihydro-8H-imidazo[2,1- c][1,4]oxazin-3-yl)-N-(5-(2-(3,3- dimethylazetidin-1- yl)acetamido)-2-methylpyridin- 3-yl)pyrazolo[5,1-b]thiazole-7- carboxamide |
| 416 | 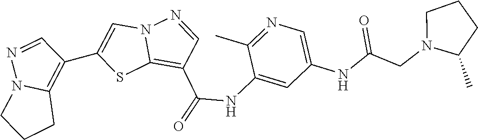 [SMILES: Cc1ncc(NC(=O)CN2CCC[C@@H]2C)cc1NC(=O)c1cnn2cc(-c3cnn4c3CCC4)sc12] | (S)-2-(5,6-dihydro-4H- pyrrolo[1,2-b]pyrazol-3-yl)-N- (2-methyl-5-(2-(2- methylpyrrolidin-1- yl)acetamido)pyridin-3- yl)pyrazolo[5,1-b]thiazole-7- carboxamide |
| 417 | 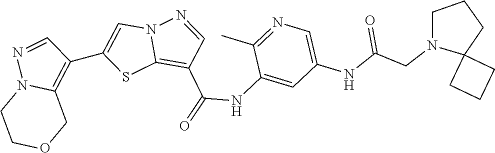 [SMILES: Cc1ncc(NC(=O)CN2CCCC23CCC3)cc1NC(=O)c1cnn2cc(-c3cnn4c3COCC4)sc12] | N-(5-(2-(5-azaspiro[3.4]octan-5- yl)acetamido)-2-methylpyridin- 3-yl)-2-(6,7-dihydro-4H- pyrazolo[5,1-c][1,4]oxazin-3- yl)pyrazolo[5,1-b]thiazole-7- carboxamide |
| 418 | 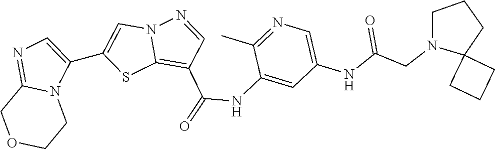 [SMILES: Cc1ncc(NC(=O)CN2CCCC23CCC3)cc1NC(=O)c1cnn2cc(-c3cnc4n3CCOC4)sc12] | N-(5-(2-(5-azaspiro[3.4]octan-5- yl)acetamido)-2-methylpyridin- 3-yl)-2-(5,6-dihydro-8H- imidazo[2,1-c][1,4]oxazin-3- yl)pyrazolo[5,1-b]thiazole-7- carboxamide |
| 419 | 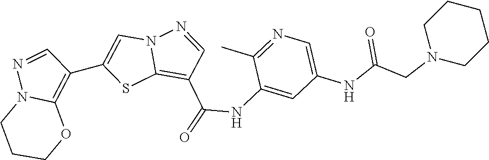 [SMILES: Cc1ncc(NC(=O)CN2CCCCC2)cc1NC(=O)c1cnn2cc(-c3cnn4c3OCCC4)sc12] | 2-(6,7-dihydro-5H-pyrazolo[5,1- b][1,3]oxazin-3-yl)-N-(2-methyl- 5-(2-(piperidin-1- yl)acetamido)pyridin-3- yl)pyrazolo[5,1-b]thiazole-7- carboxamide |
| 420 | 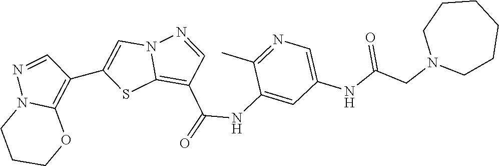 [SMILES: Cc1ncc(NC(=O)CN2CCCCCC2)cc1NC(=O)c1cnn2cc(-c3cnn4c3OCCC4)sc12] | N-(5-(2-(azepan-1- yl)acetamido)-2-methylpyridin- 3-yl)-2-(6,7-dihydro-5H- pyrazolo[5,1-b][1,3]oxazin-3- yl)pyrazolo[5,1-b]thiazole-7- carboxamide |
| 421 | 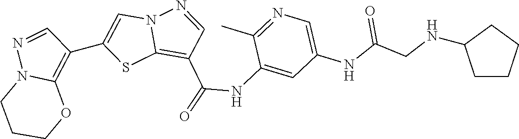 [SMILES: Cc1ncc(NC(=O)CNC2CCCC2)cc1NC(=O)c1cnn2cc(-c3cnn4c3OCCC4)sc12] | N-(5-(2- (cyclopentylamino)acetamido)- 2-methylpyridin-3-yl)-2-(6,7- dihydro-5H-pyrazolo[5,1- b][1,3]oxazin-3-yl)pyrazolo[5,1- b]thiazole-7-carboxamide |
| 422 | 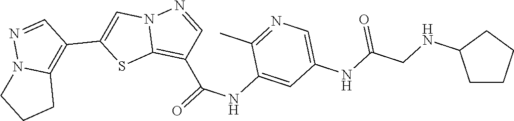 [SMILES: Cc1ncc(NC(=O)CNC2CCCC2)cc1NC(=O)c1cnn2cc(-c3cnn4c3CCC4)sc12] | N-(5-(2- (cyclopentylamino)acetamido)- 2-methylpyridin-3-yl)-2-(5,6- dihydro-4H-pyrrolo[1,2- b]pyrazol-3-yl)pyrazolo[5,1- b]thiazole-7-carboxamide |
| 423 | 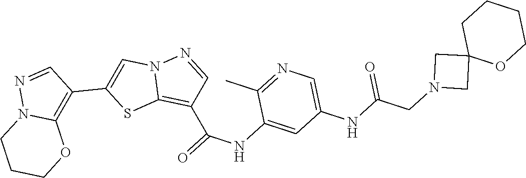 [SMILES: Cc1ncc(NC(=O)CN2CC3(CCCCO3)C2)cc1NC(=O)c1cnn2cc(-c3cnn4c3OCCC4)sc12] | N-(5-(2-(5-oxa-2- azaspiro[3.5]nonan-2- yl)acetamido)-2-methylpyridin- 3-yl)-2-(6,7-dihydro-5H- pyrazolo[5,1-b][1,3]oxazin-3- yl)pyrazolo[5,1-b]thiazole-7- carboxamide |
| 424 | 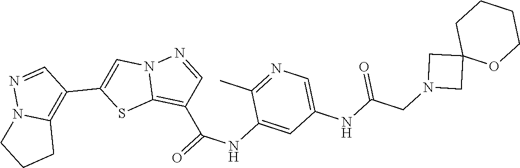 [SMILES: Cc1ncc(NC(=O)CN2CC3(CCCCO3)C2)cc1NC(=O)c1cnn2cc(-c3cnn4c3CCC4)sc12] | N-(5-(2-(5-oxa-2- azaspiro[3.5]nonan-2- yl)acetamido)-2-methylpyridin- 3-yl)-2-(5,6-dihydro-4H- pyrrolo[1,2-b]pyrazol-3- yl)pyrazolo[5,1-b]thiazole-7- carboxamide |
| 425 | 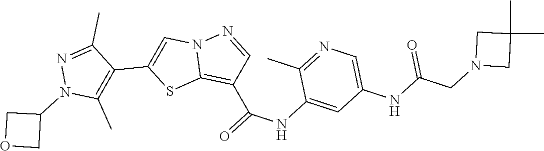 [SMILES: Cc1ncc(NC(=O)CN2CC(C)(C)C2)cc1NC(=O)c1cnn2cc(-c3c(C)nn(C4COC4)c3C)sc12] | 2-(3,5-dimethyl-1-(oxetan-3-yl)- 1H-pyrazol-4-yl)-N-(5-(2-(3,3- dimethylazetidin-1- yl)acetamido)-2-methylpyridin- 3-yl)pyrazolo[5,1-b]thiazole-7- carboxamide |
| 426 | 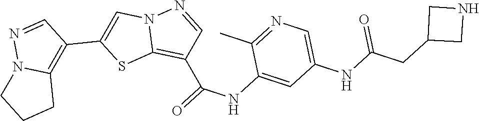 [SMILES: Cc1ncc(NC(=O)CC2CNC2)cc1NC(=O)c1cnn2cc(-c3cnn4c3CCC4)sc12] | N-(5-(2-(azetidin-3- yl)acetamido)-2-methylpyridin- 3-yl)-2-(5,6-dihydro-4H- pyrrolo[1,2-b]pyrazol-3- yl)pyrazolo[5,1-b]thiazole-7- carboxamide |
| 427 | 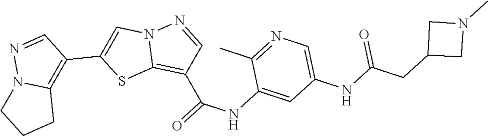 [SMILES: Cc1ncc(NC(=O)CC2CN(C)C2)cc1NC(=O)c1cnn2cc(-c3cnn4c3CCC4)sc12] | 2-(5,6-dihydro-4H-pyrrolo[1,2- b]pyrazol-3-yl)-N-(2-methyl-5- (2-(1-methylazetidin-3- yl)acetamido)pyridin-3- yl)pyrazolo[5,1-b]thiazole-7- carboxamide |
| 428 | 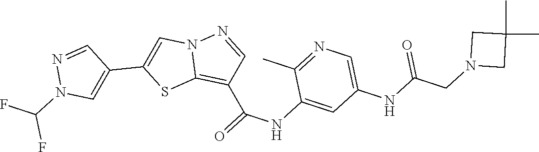 [SMILES: Cc1ncc(NC(=O)CN2CC(C)(C)C2)cc1NC(=O)c1cnn2cc(-c3cnn(C(F)F)c3)sc12] | 2-(1-(difluoromethyl)-1H- pyrazol-4-yl)-N-(5-(2-(3,3- dimethylazetidin-1- yl)acetamido)-2-methylpyridin- 3-yl)pyrazolo[5,1-b]thiazole-7- carboxamide |
| 429 | 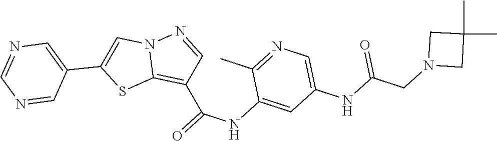 [SMILES: Cc1ncc(NC(=O)CN2CC(C)(C)C2)cc1NC(=O)c1cnn2cc(-c3cncnc3)sc12] | N-(5-(2-(3,3-dimethylazetidin-1- yl)acetamido)-2-methylpyridin- 3-yl)-2-(pyrimidin-5- yl)pyrazolo[5,1-b]thiazole-7- carboxamide |
| 430 | 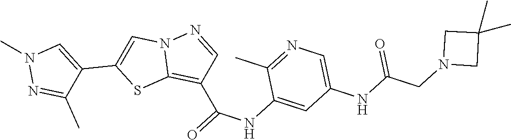 | 2-(1,3-Dimethyl-1H-pyrazol-4- yl)-N-(5-(2-(3,3- dimethylazetidin-1- yl)acetamido)-2-methylpyridin- 3-yl)pyrazolo[5,1-b]thiazole-7- carboxamide |
| 431 | 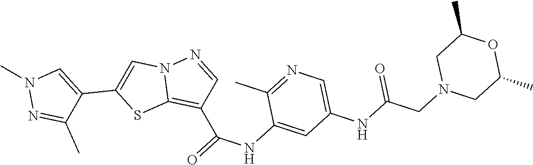 | 2-(1,3-Dimethyl-1H-pyrazol-4- yl)-N-(5-(2-trans-2,6- dimethylmorpholino)acetamido)- 2-methylpyridin-3- yl)pyrazolo[5,1-b]thiazole-7- carboxamide |
| 432 | 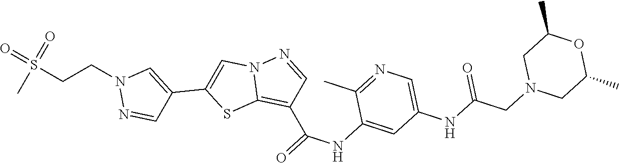 [SMILES: Cc1ncc(NC(=O)CN2C[C@@H](C)O[C@H](C)C2)cc1NC(=O)c1cnn2cc(-c3cnn(CCS(C)(=O)=O)c3)sc12] | N-(5-(2-trans-2,6- dimethylmorpholino)acetamido)- 2-methylpyridin-3-yl)-2-(1-(2- (methylsulfonyl)ethyl)-1H- pyrazol-4-yl)pyrazolo[5,1- b]thiazole-7-carboxamide |
| 433 | 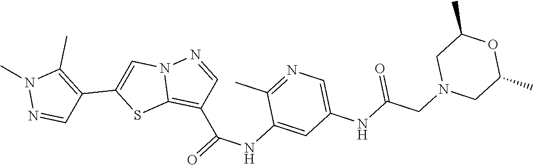 | 2-(1,5-Dimethyl-1H-pyrazol-4- yl)-N-(5-(2-trans-2,6- dimethylmorpholino)acetamido)- 2-methylpyridin-3- yl)pyrazolo[5,1-b]thiazole-7- carboxamide |
| 434 | 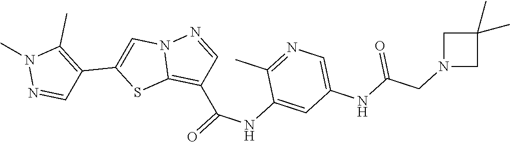 | 2-(1,5-Dimethyl-1H-pyrazol-4- yl)-N-(5-(2-(3,3- dimethylazetidin-1- yl)acetamido)-2-methylpyridin- 3-yl)pyrazolo[5,1-b]thiazole-7- carboxamide |
| 435 | 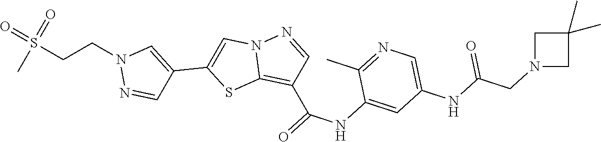 [SMILES: Cc1ncc(NC(=O)CN2CC(C)(C)C2)cc1NC(=O)c1cnn2cc(-c3cnn(CCS(C)(=O)=O)c3)sc12] | N-(5-(2-(3,3-Dimethylazetidin- 1-yl)acetamido)-2- methylpyridin-3-yl)-2-(1-(2- (methylsulfonyl)ethyl)-1H- pyrazol-4-yl)pyrazolo[5,1- b]thiazole-7-carboxamide |
| 436 | 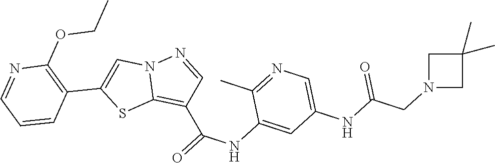 [SMILES: CCOc1ncccc1-c1cn2ncc(C(=O)Nc3cc(NC(=O)CN4CC(C)(C)C4)cnc3C)c2s1] | N-(5-(2-(3,3-Dimethylazetidin- 1-yl)acetamido)-2- methylpyridin-3-yl)-2-(2- ethoxypyridin-3-yl)pyrazolo[5,1- b]thiazole-7-carboxamide |
| 437 | 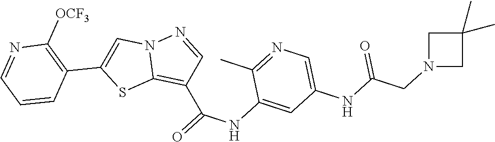 [SMILES: Cc1ncc(NC(=O)CN2CC(C)(C)C2)cc1NC(=O)c1cnn2cc(-c3cccnc3OC(F)(F)F)sc12] | N-(5-(2-(3,3-dimethylazetidin-1- yl)acetamido)-2-methylpyridin- 3-yl)-2-(2- (trifluoromethoxy)pyridin-3- yl)pyrazolo[5,1-b]thiazole-7- carboxamide |
| 438 | 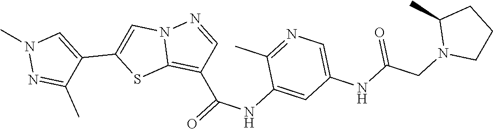 [SMILES: Cc1ncc(NC(=O)CN2CCC[C@@H]2C)cc1NC(=O)c1cnn2cc(-c3cn(C)nc3C)sc12] | (S)-2-(1,3-dimethyl-1H-pyrazol- 4-yl)-N-(2-methyl-5-(2-(2- methylpyrrolidin-1- yl)acetamido)pyridin-3- yl)pyrazolo[5,1-b]thiazole-7- carboxamide |
| 439 | 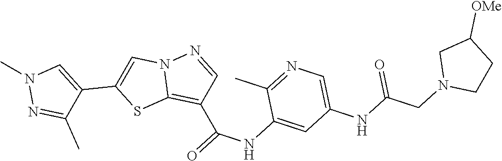 [SMILES: COC1CCN(CC(=O)Nc2cnc(C)c(NC(=O)c3cnn4cc(-c5cn(C)nc5C)sc34)c2)C1] | 2-(1,3-Dimethyl-1H-pyrazol-4- yl)-N-(5-(2-(3- methoxypyrrolidin-1- yl)acetamido)-2-methylpyridin- 3-yl)pyrazolo[5,1-b]thiazole-7- carboxamide |
| 440 | 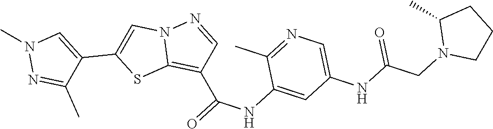 [SMILES: Cc1ncc(NC(=O)CN2CCC[C@H]2C)cc1NC(=O)c1cnn2cc(-c3cn(C)nc3C)sc12] | (R)-2-(1,3-dimethyl-1H-pyrazol- 4-yl)-N-(2-methyl-5-(2-(2- methylpyrrolidin-1- yl)acetamido)pyridin-3- yl)pyrazolo[5,1-b]thiazole-7- carboxamide |
| 441 | 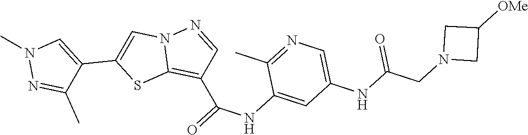 [SMILES: COC1CN(CC(=O)Nc2cnc(C)c(NC(=O)c3cnn4cc(-c5cn(C)nc5C)sc34)c2)C1] | 2-(1,3-Dimethyl-1H-pyrazol-4- yl)-N-(5-(2-(3-methoxyazetidin- 1-yl)acetamido)-2- methylpyridin-3- yl)pyrazolo[5,1-b]thiazole-7- carboxamide |
| 442 | 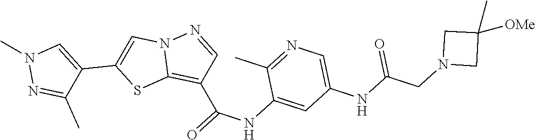 [SMILES: COC1(C)CN(CC(=O)Nc2cnc(C)c(NC(=O)c3cnn4cc(-c5cn(C)nc5C)sc34)c2)C1] | 2-(1,3-Dimethyl-1H-pyrazol-4- yl)-N-(5-(2-(3-methoxy-3- methylazetidin-1-yl)acetamido)- 2-methylpyridin-3- yl)pyrazolo[5,1-b]thiazole-7- carboxamide |
| 443 | 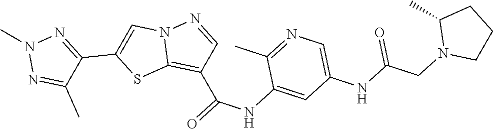 [SMILES: Cc1ncc(NC(=O)CN2CCC[C@H]2C)cc1NC(=O)c1cnn2cc(-c3nn(C)nc3C)sc12] | (R)-2-(2,5-dimethyl-2H-1,2,3- triazol-4-yl)-N-(2-methyl-5-(2- (2-methylpyrrolidin-1- yl)acetamido)pyridin-3- yl)pyrazolo[5,1-b]thiazole-7- carboxamide |
| 444 | 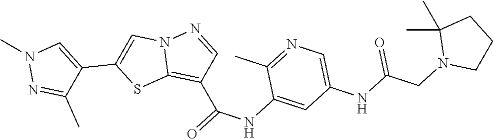 [SMILES: Cc1ncc(NC(=O)CN2CCCC2(C)C)cc1NC(=O)c1cnn2cc(-c3cn(C)nc3C)sc12] | 2-(1,3-Dimethyl-1H-pyrazol-4- yl)-N-(5-(2-(2,2- dimethylpyrrolidin-1- yl)acetamido)-2-methylpyridin- 3-yl)pyrazolo[5,1-b]thiazole-7- carboxamide |
| 445 | 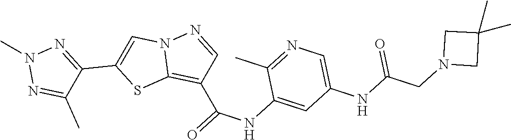 | 2-(2,5-Dimethyl-2H-1,2,3- triazol-4-yl)-N-(5-(2-(3,3- dimethylazetidin-1- yl)acetamido)-2-methylpyridin- 3-yl)pyrazolo[5,1-b]thiazole-7- carboxamide |
| 446 | 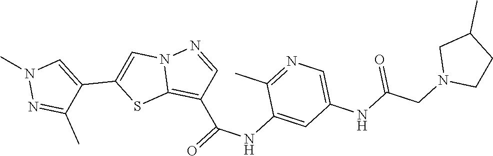 [SMILES: Cc1ncc(NC(=O)CN2CCC(C)C2)cc1NC(=O)c1cnn2cc(-c3cn(C)nc3C)sc12] | 2-(1,3-Dimethyl-1H-pyrazol-4- yl)-N-(2-methyl-5-(2-(3- methylpyrrolidin-1- yl)acetamido)pyridin-3- yl)pyrazolo[5,1-b]thiazole-7- carboxamide |
| 447 | 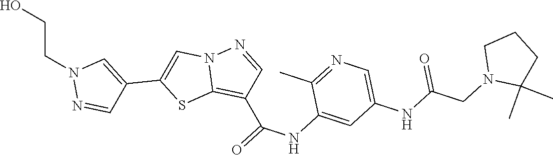 [SMILES: Cc1ncc(NC(=O)CN2CCCC2(C)C)cc1NC(=O)c1cnn2cc(-c3cnn(CCO)c3)sc12] | N-(5-(2-(2,2-dimethylpyrrolidin- 1-yl)acetamido)-2- methylpyridin-3-yl)-2-(1-(2- hydroxyethyl)-1H-pyrazol-4- yl)pyrazolo[5,1-b]thiazole-7- carboxamide |
| 448 | 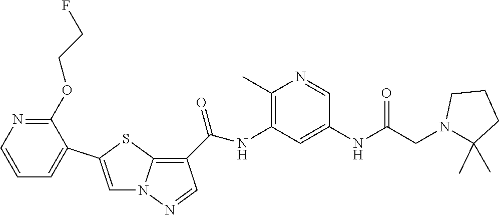 [SMILES: Cc1ncc(NC(=O)CN2CCCC2(C)C)cc1NC(=O)c1cnn2cc(-c3cccnc3OCCF)sc12] | N-(5-(2-(2,2-dimethylpyrrolidin- 1-yl)acetamido)-2- methylpyridin-3-yl)-2-(2-(2- fluoroethoxy)pyridin-3- yl)pyrazolo[5,1-b]thiazole-7- carboxamide |
| 449 | 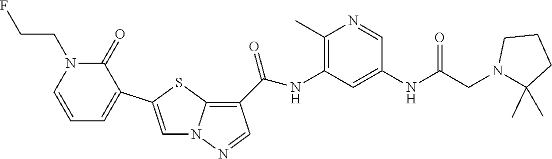 [SMILES: Cc1ncc(NC(=O)CN2CCCC2(C)C)cc1NC(=O)c1cnn2cc(-c3cccn(CCF)c3=O)sc12] | N-(5-(2-(2,2-dimethylpyrrolidin- 1-yl)acetamido)-2- methylpyridin-3-yl)-2-(1-(2- fluoroethyl)-2-oxo-1,2- dihydropyridin-3- yl)pyrazolo[5,1-b]thiazole-7- carboxamide |
| 450 | 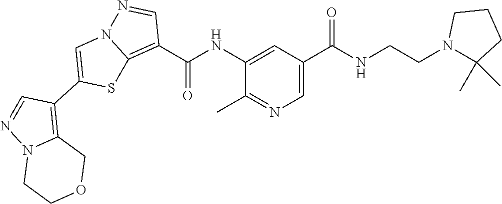 [SMILES: Cc1ncc(C(=O)NCCN2CCCC2(C)C)cc1NC(=O)c1cnn2cc(-c3cnn4c3COCC4)sc12] | 2-(6,7-dihydro-4H-pyrazolo[5,1- c][1,4]oxazin-3-yl)-N-(5-((2- (2,2-dimethylpyrrolidin-1- yl)ethyl)carbamoyl)-2- methylpyridin-3- yl)pyrazolo[5,1-b]thiazole-7- carboxamide |
| 451 | 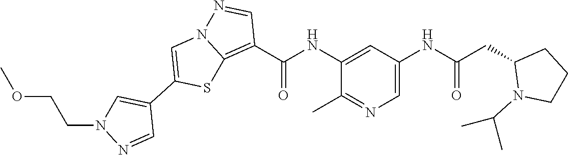 [SMILES: COCCn1cc(-c2cn3ncc(C(=O)Nc4cc(NC(=O)C[C@@H]5CCCN5C(C)C)cnc4C)c3s2)cn1] | (S)-N-(5-(2-(1- isopropylpyrrolidin-2- yl)acetamido)-2-methylpyridin- 3-yl)-2-(1-(2-methoxyethyl)-1H- pyrazol-4-yl)pyrazolo[5,1- b]thiazole-7-carboxamide |
| 452 | 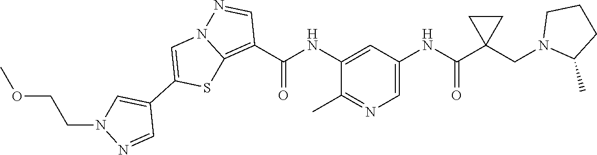 [SMILES: COCCn1cc(-c2cn3ncc(C(=O)Nc4cc(NC(=O)C5(CN6CCC[C@@H]6C)CC5)cnc4C)c3s2)cn1] | (S)-2-(1-(2-methoxyethyl)-1H- pyrazol-4-yl)-N-(2-methyl-5-(1- ((2-methylpyrrolidin-1- yl)methyl)cyclopropanecarboxa mido)pyridin-3-yl)pyrazolo[5,1- b]thiazole-7-carboxamide |
| 453 | 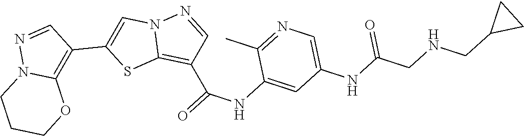 [SMILES: Cc1ncc(NC(=O)CNCC2CC2)cc1NC(=O)c1cnn2cc(-c3cnn4c3OCCC4)sc12] | N-(5-(2- ((cyclopropylmethyl)amino)acet amido)-2-methylpyridin-3-yl)-2- (6,7-dihydro-5H-pyrazolo[5,1- b][1,3]oxazin-3-yl)pyrazolo[5,1- b]thiazole-7-carboxamide |
| 454 | 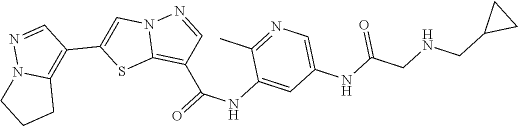 [SMILES: Cc1ncc(NC(=O)CNCC2CC2)cc1NC(=O)c1cnn2cc(-c3cnn4c3CCC4)sc12] | N-(5-(2- ((cyclopropylmethyl)amino)acet amido)-2-methylpyridin-3-yl)-2- (5,6-dihydro-4H-pyrrolo[1,2- b]pyrazol-3-yl)pyrazolo[5,1- b]thiazole-7-carboxamide |
| 455 | 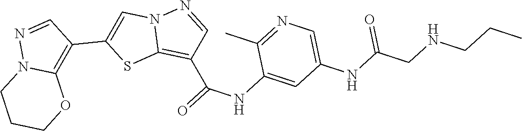 [SMILES: CCCNCC(=O)Nc1cnc(C)c(NC(=O)c2cnn3cc(-c4cnn5c4OCCC5)sc23)c1] | 2-(6,7-dihydro-5H-pyrazolo[5,1- b][1,3]oxazin-3-yl)-N-(2-methyl- 5-(2- (propylamino)acetamido)pyridin- 3-yl)pyrazolo[5,1-b]thiazole-7- carboxamide |
| 456 | 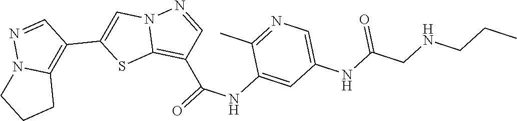 [SMILES: CCCNCC(=O)Nc1cnc(C)c(NC(=O)c2cnn3cc(-c4cnn5c4CCC5)sc23)c1] | 2-(5,6-dihydro-4H-pyrrolo[1,2- b]pyrazol-3-yl)-N-(2-methyl-5- (2- (propylamino)acetamido)pyridin- 3-yl)pyrazolo[5,1-b]thiazole-7- carboxamide |
| 457 | 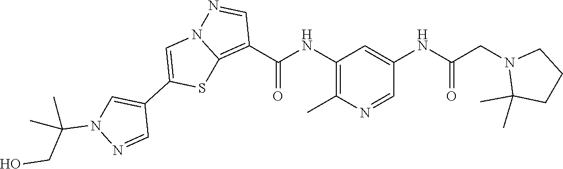 [SMILES: Cc1ncc(NC(=O)CN2CCCC2(C)C)cc1NC(=O)c1cnn2cc(-c3cnn(C(C)(C)CO)c3)sc12] | N-(5-(2-(2,2-dimethylpyrrolidin- 1-yl)acetamido)-2- methylpyridin-3-yl)-2-(1-(1- hydroxy-2-methylpropan-2-yl)- 1H-pyrazol-4-yl)pyrazolo[5,1- b]thiazole-7-carboxamide |
| 458 | 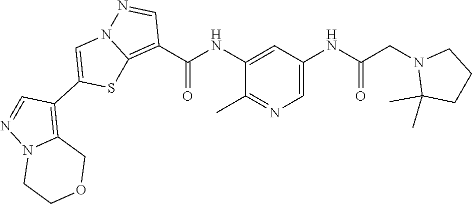 [SMILES: Cc1ncc(NC(=O)CN2CCCC2(C)C)cc1NC(=O)c1cnn2cc(-c3cnn4c3COCC4)sc12] | 2-(6,7-dihydro-4H-pyrazolo[5,1- c][1,4]oxazin-3-yl)-N-(5-(2-(2,2- dimethylpyrrolidin-1- yl)acetamido)-2-methylpyridin- 3-yl)pyrazolo[5,1-b]thiazole-7- carboxamide |
| 459 | 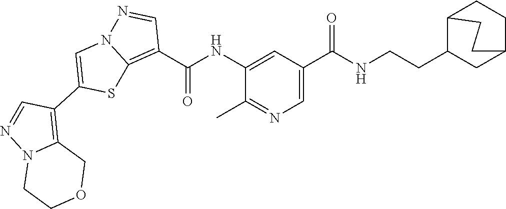 [SMILES: Cc1ncc(C(=O)NCCC2CC3CCC2CC3)cc1NC(=O)c1cnn2cc(-c3cnn4c3COCC4)sc12] | N-(5-((2-(2- azabicyclo[2.2.2]octan-2- yl)ethyl)carbamoyl)-2- methylpyridin-3-yl)-2-(6,7- dihydro-4H-pyrazolo[5,1- c][1,4]oxazin-3-yl)pyrazolo[5,1- b]thiazole-7-carboxamide |
| 460 | 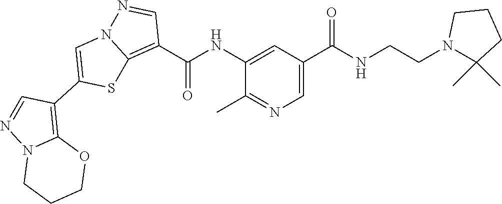 [SMILES: Cc1ncc(C(=O)NCCN2CCCC2(C)C)cc1NC(=O)c1cnn2cc(-c3cnn4c3OCCC4)sc12] | 2-(6,7-dihydro-5H-pyrazolo[5,1- b][1,3]oxazin-3-yl)-N-(5-((2- (2,2-dimethylpyrrolidin-1- yl)ethyl)carbamoyl)-2- methylpyridin-3- yl)pyrazolo[5,1-b]thiazole-7- carboxamide |
| 461 | 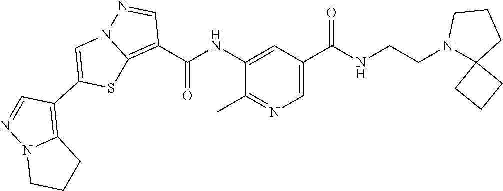 [SMILES: Cc1ncc(C(=O)NCCN2CCCC23CCC3)cc1NC(=O)c1cnn2cc(-c3cnn4c3CCC4)sc12] | N-(5-((2-(5-azaspiro[3.4]octan- 5-yl)ethyl)carbamoyl)-2- methylpyridin-3-yl)-2-(5,6- dihydro-4H-pyrrolo[1,2- b]pyrazol-3-yl)pyrazolo[5,1- b]thiazole-7-carboxamide |
| 462 | 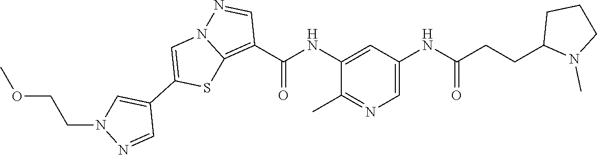 [SMILES: COCCn1cc(-c2cn3ncc(C(=O)Nc4cc(NC(=O)CCC5CCCN5C)cnc4C)c3s2)cn1] | 2-(1-(2-methoxyethyl)-1H- pyrazol-4-yl)-N-(2-methyl-5-(3- (1-methylpyrrolidin-2- yl)propanamido)pyridin-3- yl)pyrazolo[5,1-b]thiazole-7- carboxamide |
| 463 | 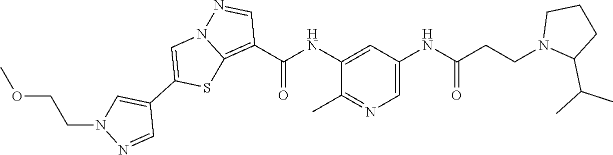 [SMILES: COCCn1cc(-c2cn3ncc(C(=O)Nc4cc(NC(=O)CCN5CCCC5C(C)C)cnc4C)c3s2)cn1] | N-(5-(3-(1-isopropylpyrrolidin- 2-yl)propanamido)-2- methylpyridin-3-yl)-2-(1-(2- methoxyethyl)-1H-pyrazol-4- yl)pyrazolo[5,1-b]thiazole-7- carboxamide |
| 464 | 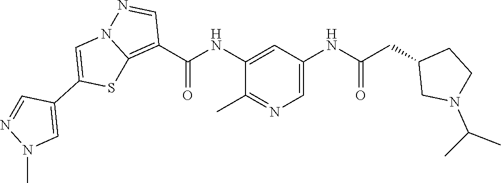 [SMILES: Cc1ncc(NC(=O)C[C@@H]2CCN(C(C)C)C2)cc1NC(=O)c1cnn2cc(-c3cnn(C)c3)sc12] | (S)-N-(5-(2-(1- isopropylpyrrolidin-3- yl)acetamido)-2-methylpyridin- 3-yl)-2-(1-methyl-1H-pyrazol-4- yl)pyrazolo[5,1-b]thiazole-7- carboxamide |
| 465 | 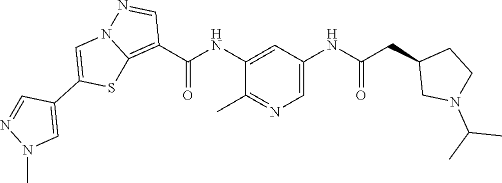 [SMILES: Cc1ncc(NC(=O)C[C@H]2CCN(C(C)C)C2)cc1NC(=O)c1cnn2cc(-c3cnn(C)c3)sc12] | (R)-N-(5-(2-(1- isopropylpyrrolidin-3- yl)acetamido)-2-methylpyridin- 3-yl)-2-(1-methyl-1H-pyrazol-4- yl)pyrazolo[5,1-b]thiazole-7- carboxamide |
| 466 | 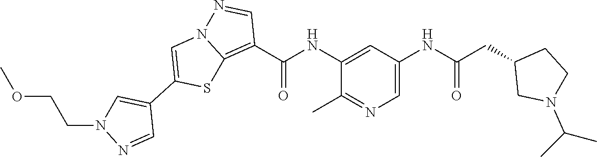 [SMILES: COCCn1cc(-c2cn3ncc(C(=O)Nc4cc(NC(=O)C[C@@H]5CCN(C(C)C)C5)cnc4C)c3s2)cn1] | (S)-N-(5-(2-(1- isopropylpyrrolidin-3- yl)acetamido)-2-methylpyridin- 3-yl)-2-(1-(2-methoxyethyl)-1H- pyrazol-4-yl)pyrazolo[5,1- b]thiazole-7-carboxamide |
| 467 | 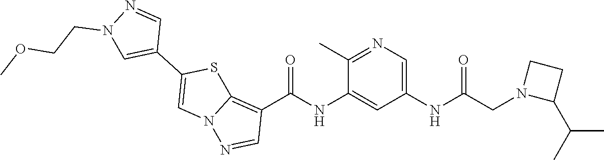 [SMILES: COCCn1cc(-c2cn3ncc(C(=O)Nc4cc(NC(=O)CN5CCC5C(C)C)cnc4C)c3s2)cn1] | N-(5-(2-(2-isopropylazetidin-1- yl)acetamido)-2-methylpyridin- 3-yl)-2-(1-(2-methoxyethyl)-1H- pyrazol-4-yl)pyrazolo[5,1- b]thiazole-7-carboxamide |
| 468 | 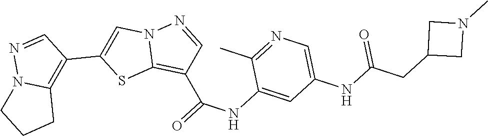 [SMILES: Cc1ncc(NC(=O)CC2CN(C)C2)cc1NC(=O)c1cnn2cc(-c3cnn4c3CCC4)sc12] | 2-(5,6-dihydro-4H-pyrrolo[1,2- b]pyrazol-3-yl)-N-(2-methyl-5- (2-(1-methylazetidin-3- yl)acetamido)pyridin-3- yl)pyrazolo[5,1-b]thiazole-7- carboxamide |
| 469 | 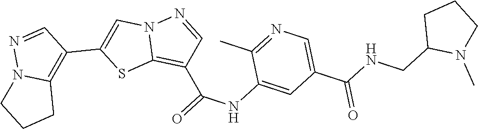 [SMILES: Cc1ncc(C(=O)NCC2CCCN2C)cc1NC(=O)c1cnn2cc(-c3cnn4c3CCC4)sc12] | 2-(5,6-dihydro-4H-pyrrolo[1,2- b]pyrazol-3-yl)-N-(2-methyl-5- (((1-methylpyrrolidin-2- yl)methyl)carbamoyl)pyridin-3- yl)pyrazolo[5,1-b]thiazole-7- carboxamide |
| 470 | 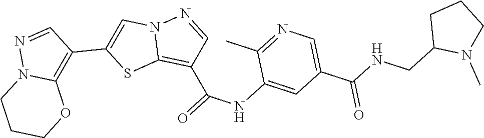 [SMILES: Cc1ncc(C(=O)NCC2CCCN2C)cc1NC(=O)c1cnn2cc(-c3cnn4c3OCCC4)sc12] | 2-(6,7-dihydro-5H-pyrazolo[5,1- b][1,3]oxazin-3-yl)-N-(2-methyl- 5-(((1-methylpyrrolidin-2- yl)methyl)carbamoyl)pyridin-3- yl)pyrazolo[5,1-b]thiazole-7- carboxamide |
| 471 | 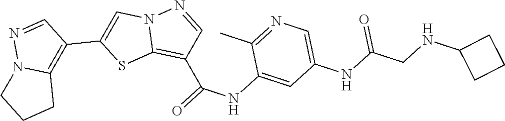 [SMILES: Cc1ncc(NC(=O)CNC2CCC2)cc1NC(=O)c1cnn2cc(-c3cnn4c3CCC4)sc12] | N-(5-(2- (cyclobutylamino)acetamido)- 2-methylpyridin-3-yl)-2-(5,6- dihydro-4H-pyrrolo[1,2- b]pyrazol-3-yl)pyrazolo[5,1- b]thiazole-7-carboxamide |
| 472 | 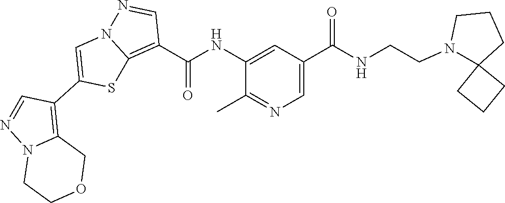 [SMILES: Cc1ncc(C(=O)NCCN2CCCC23CCC3)cc1NC(=O)c1cnn2cc(-c3cnn4c3COCC4)sc12] | N-(5-((2-(5- azaspiro[3.4]octan-5- yl)ethyl)carbamoyl)-2- methylpyridin-3-yl)-2-(6,7- dihydro-4H-pyrazolo[5,1- c][1,4]oxazin-3- yl)pyrazolo[5,1-b]thiazole-7- carboxamide |
| 473 | 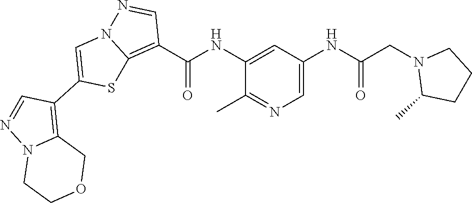 [SMILES: Cc1ncc(NC(=O)CN2CCC[C@@H]2C)cc1NC(=O)c1cnn2cc(-c3cnn4c3COCC4)sc12] | (S)-2-(6,7-dihydro-4H- pyrazolo[5,1-c][1,4]oxazin-3- yl)-N-(2-methyl-5-(2-(2- methylpyrrolidin-1- yl)acetamido)pyridin-3- yl)pyrazolo[5,1-b]thiazole-7- carboxamide |
| 474 | 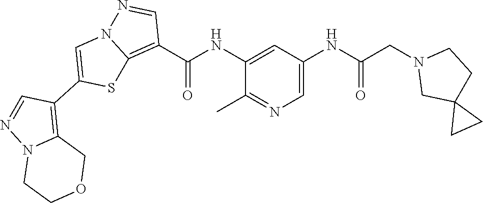 [SMILES: Cc1ncc(NC(=O)CN2CCC3(CC3)C2)cc1NC(=O)c1cnn2cc(-c3cnn4c3COCC4)sc12] | N-(5-(2-(5- azaspiro[2.4]heptan-5- yl)acetamido)-2- methylpyridin-3-yl)-2-(6,7- dihydro-4H-pyrazolo[5,1- c][1,4]oxazin-3- yl)pyrazolo[5,1-b]thiazole-7- carboxamide |
| 475 | 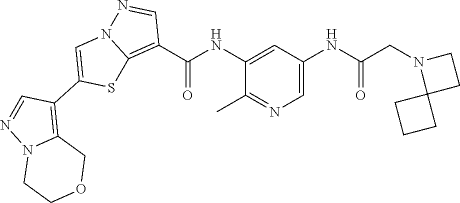 [SMILES: Cc1ncc(NC(=O)CN2CCC23CCC3)cc1NC(=O)c1cnn2cc(-c3cnn4c3COCC4)sc12] | N-(5-(2-(1- azaspiro[3.3]heptan-1- yl)acetamido)-2- methylpyridin-3-yl)-2-(6,7- dihydro-4H-pyrazolo[5,1- c][1,4]oxazin-3- yl)pyrazolo[5,1-b]thiazole-7- carboxamide |
| 476 | 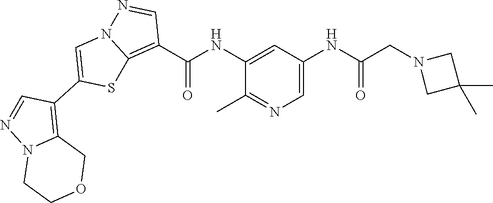 [SMILES: Cc1ncc(NC(=O)CN2CC(C)(C)C2)cc1NC(=O)c1cnn2cc(-c3cnn4c3COCC4)sc12] | 2-(6,7-dihydro-4H- pyrazolo[5,1-c][1,4]oxazin-3- yl)-N-(5-(2-(3,3- dimethylazetidin-1- yl)acetamido)-2- methylpyridin-3- yl)pyrazolo[5,1-b]thiazole-7- carboxamide |
| 477 | 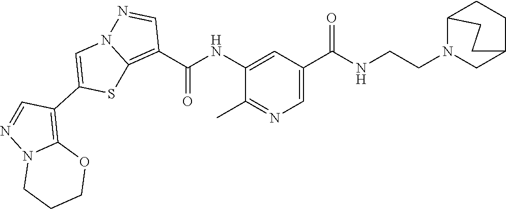 [SMILES: Cc1ncc(C(=O)NCCN2CC3CCC2CC3)cc1NC(=O)c1cnn2cc(-c3cnn4c3OCCC4)sc12] | N-(5-((2-(2- azabicyclo[2.2.2]octan-2- yl)ethyl)carbamoyl)-2- methylpyridin-3-yl)-2-(6,7- dihydro-5H-pyrazolo[5,1- b][1,3]oxazin-3- yl)pyrazolo[5,1-b]thiazole-7- carboxamide |
| 478 | 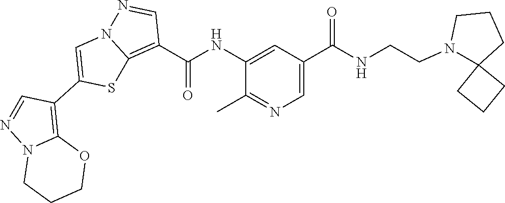 [SMILES: Cc1ncc(C(=O)NCCN2CCCC23CCC3)cc1NC(=O)c1cnn2cc(-c3cnn4c3OCCC4)sc12] | N-(5-((2-(5- azaspiro[3.4]octan-5- yl)ethyl)carbamoyl)-2- methylpyridin-3-yl)-2-(6,7- dihydro-5H-pyrazolo[5,1- b][1,3]oxazin-3- yl)pyrazolo[5,1-b]thiazole-7- carboxamide |
| 479 | 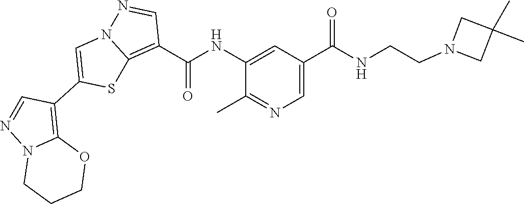 [SMILES: Cc1ncc(C(=O)NCCN2CC(C)(C)C2)cc1NC(=O)c1cnn2cc(-c3cnn4c3OCCC4)sc12] | 2-(6,7-dihydro-5H- pyrazolo[5,1-b][1,3]oxazin-3- yl)-N-(5-((2-(3,3- dimethylazetidin-1- yl)ethyl)carbamoyl)-2- methylpyridin-3- yl)pyrazolo[5,1-b]thiazole-7- carboxamide |
| 480 | 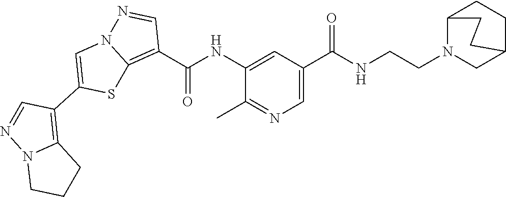 [SMILES: Cc1ncc(C(=O)NCCN2CC3CCC2CC3)cc1NC(=O)c1cnn2cc(-c3cnn4c3CCC4)sc12] | N-(5-((2-(2- azabicyclo[2.2.2]octan-2- yl)ethyl)carbamoyl)-2- methylpyridin-3-yl)-2-(5,6- dihydro-4H-pyrrolo[1,2- b]pyrazol-3-yl)pyrazolo[5,1- b]thiazole-7-carboxamide |
| 481 | 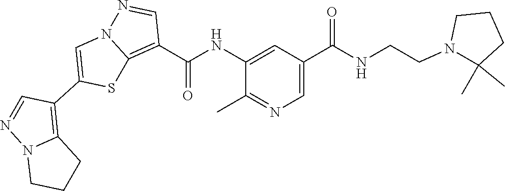 [SMILES: Cc1ncc(C(=O)NCCN2CCCC2(C)C)cc1NC(=O)c1cnn2cc(-c3cnn4c3CCC4)sc12] | (S)-N-(5-(2-(1- isopropylpyrrolidin-3- yl)acetamido)-2- methylpyridin-3-yl)-2-(1-(2- methoxyethyl)-1H-pyrazol-4- yl)pyrazolo[5,1-b]thiazole-7- carboxamide |
| 482 | 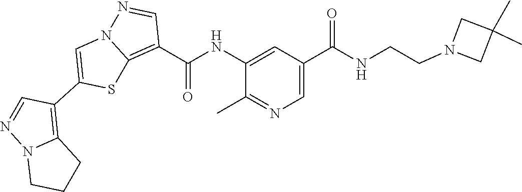 [SMILES: Cc1ncc(C(=O)NCCN2CC(C)(C)C2)cc1NC(=O)c1cnn2cc(-c3cnn4c3CCC4)sc12] | 2-(5,6-dihydro-4H- pyrrolo[1,2-b]pyrazol-3-yl)- N-(5-((2-(3,3- dimethylazetidin-1- yl)ethyl)carbamoyl)-2- methylpyridin-3- yl)pyrazolo[5,1-b]thiazole-7- carboxamide |
| 483 | 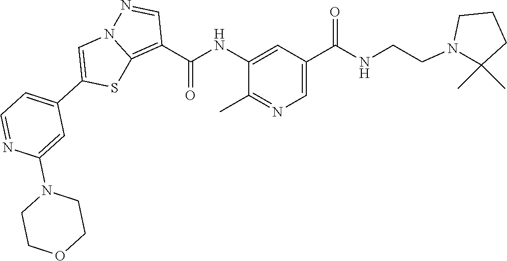 [SMILES: Cc1ncc(C(=O)NCCN2CCCC2(C)C)cc1NC(=O)c1cnn2cc(-c3ccnc(N4CCOCC4)c3)sc12] | N-(5-((2-(2,2- dimethylpyrrolidin-1- yl)ethyl)carbamoyl)-2- methylpyridin-3-yl)-2-(2- morpholinopyridin-4- yl)pyrazolo[5,1-b]thiazole-7- carboxamide |
| 484 | 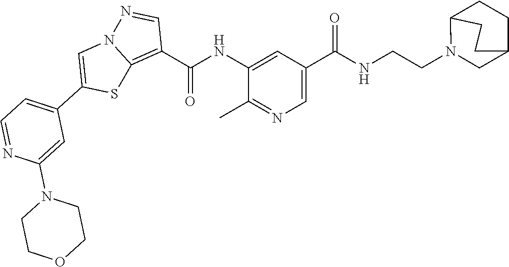 [SMILES: Cc1ncc(C(=O)NCCN2CC3CCC2CC3)cc1NC(=O)c1cnn2cc(-c3ccnc(N4CCOCC4)c3)sc12] | N-(5-((2-(2- azabicyclo[2.2.2]octan-2- yl)ethyl)carbamoyl)-2- methylpyridin-3-yl)-2-(2- morpholinopyridin-4- yl)pyrazolo[5,1-b]thiazole-7- carboxamide |
| 485 | 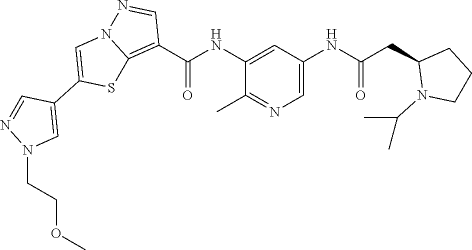 [SMILES: COCCn1cc(-c2cn3ncc(C(=O)Nc4cc(NC(=O)C[C@H]5CCCN5C(C)C)cnc4C)c3s2)cn1] | (R)-N-(5-(2-(1- isopropylpyrrolidin-2- yl)acetamido)-2- methylpyridin-3-yl)-2-(1-(2- methoxyethyl)-1H-pyrazol-4- yl)pyrazolo[5,1-b]thiazole-7- carboxamide |
| 486 | 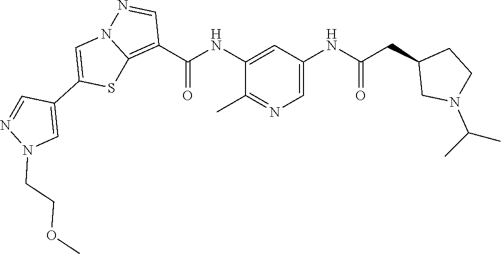 [SMILES: COCCn1cc(-c2cn3ncc(C(=O)Nc4cc(NC(=O)C[C@H]5CCN(C(C)C)C5)cnc4C)c3s2)cn1] | (R)-N-(5-(2-(1- isopropylpyrrolidin-3- yl)acetamido)-2- methylpyridin-3-yl)-2-(1-(2- methoxyethyl)-1H-pyrazol-4- yl)pyrazolo[5,1-b]thiazole-7- carboxamide |
| 487 | 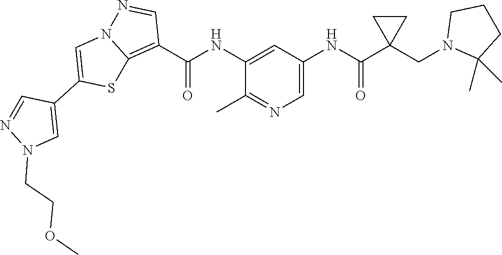 [SMILES: COCCn1cc(-c2cn3ncc(C(=O)Nc4cc(NC(=O)C5(CN6CCCC6(C)C)CC5)cnc4C)c3s2)cn1] | N-(5-(1-((2,2- dimethylpyrrolidin-1- yl)methyl)cyclopropane- carboxamido)-2- methylpyridin-3-yl)-2-(1-(2- methoxyethyl)-1H-pyrazol-4- yl)pyrazolo[5,1-b]thiazole-7- carboxamide |
| 488 | 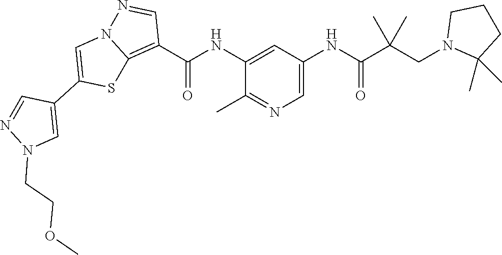 [SMILES: COCCn1cc(-c2cn3ncc(C(=O)Nc4cc(NC(=O)C(C)(C)CN5CCCC5(C)C)cnc4C)c3s2)cn1] | N-(5-(3-(2,2- dimethylpyrrolidin-1-yl)-2,2- dimethyl-propanamido)-2- methylpyridin-3-yl)-2-(1-(2- methoxyethyl)-1H-pyrazol-4- yl)pyrazolo[5,1-b]thiazole-7- carboxamide |
| 489 | 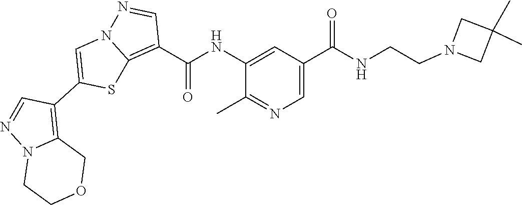 [SMILES: Cc1ncc(C(=O)NCCN2CC(C)(C)C2)cc1NC(=O)c1cnn2cc(-c3cnn4c3COCC4)sc12] | 2-(6,7-dihydro-4H- pyrazolo[5,1-c][1,4]oxazin-3- yl)-N-(5-((2-(3,3- dimethylazetidin-1- yl)ethyl)carbamoyl)-2- methylpyridin-3- yl)pyrazolo[5,1-b]thiazole-7- carboxamide |
| 490 | 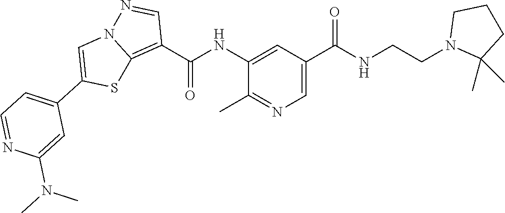 [SMILES: Cc1ncc(C(=O)NCCN2CCCC2(C)C)cc1NC(=O)c1cnn2cc(-c3ccnc(N(C)C)c3)sc12] | 2-(2-(dimethylamino)pyridin- 4-yl)-N-(5-((2-(2,2- dimethylpyrrolidin-1- yl)ethyl)carbamoyl)-2- methylpyridin-3- yl)pyrazolo[5,1-b]thiazole-7- carboxamide |
| 491 | 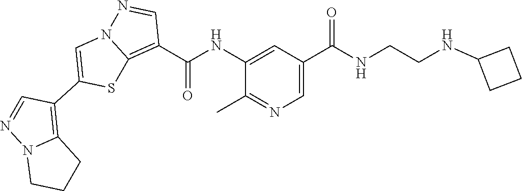 [SMILES: Cc1ncc(C(=O)NCCNC2CCC2)cc1NC(=O)c1cnn2cc(-c3cnn4c3CCC4)sc12] | N-(5-((2- (cyclobutylamino)ethyl)carba moyl)-2-methylpyridin-3-yl)- 2-(5,6-dihydro-4H- pyrrolo[1,2-b]pyrazol-3- yl)pyrazolo[5,1-b]thiazole-7- carboxamide |
| 492 | 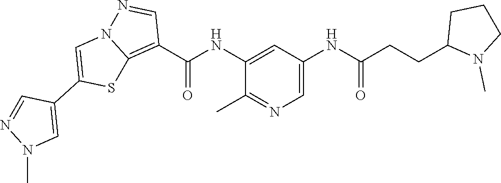 [SMILES: Cc1ncc(NC(=O)CCC2CCCN2C)cc1NC(=O)c1cnn2cc(-c3cnn(C)c3)sc12] | 2-(1-methyl-1H-pyrazol-4- yl)-N-(2-methyl-5-(3-(1- methylpyrrolidin-2- yl)propanamido)pyridin-3- yl)pyrazolo[5,1-b]thiazole-7- carboxamide |
| 493 | 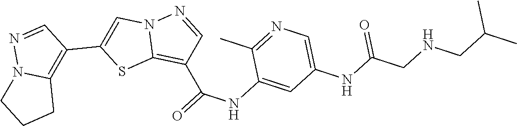 [SMILES: Cc1ncc(NC(=O)CNCC(C)C)cc1NC(=O)c1cnn2cc(-c3cnn4c3CCC4)sc12] | 2-(5,6-dihydro-4H- pyrrolo[1,2-b]pyrazol-3-yl)- N-(5-(2- (isobutylamino)acetamido)-2- methylpyridin-3- yl)pyrazolo[5,1-b]thiazole-7- carboxamide |
| 494 | 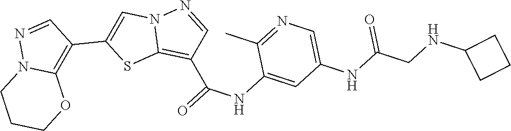 [SMILES: Cc1ncc(NC(=O)CNC2CCC2)cc1NC(=O)c1cnn2cc(-c3cnn4c3OCCC4)sc12] | N-(5-(2- (cyclobutylamino)acetamido)- 2-methylpyridin-3-yl)-2-(6,7- dihydro-5H-pyrazolo[5,1- b][1,3]oxazin-3- yl)pyrazolo[5,1-b]thiazole-7- carboxamide |
| 495 | 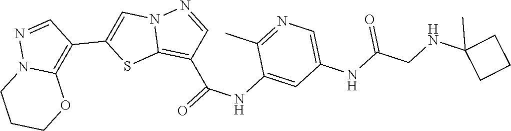 [SMILES: Cc1ncc(NC(=O)CNC2(C)CCC2)cc1NC(=O)c1cnn2cc(-c3cnn4c3OCCC4)sc12] | 2-(6,7-dihydro-5H- pyrazolo[5,1-b][1,3]oxazin-3- yl)-N-(2-methyl-5-(2-((1- methylcyclobutyl)amino)acet amido)pyridin-3- yl)pyrazolo[5,1-b]thiazole-7- carboxamide |
| 496 | 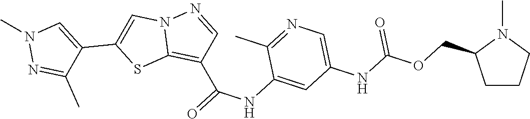 [SMILES: Cc1ncc(NC(=O)OC[C@@H]2CCCN2C)cc1NC(=O)c1cnn2cc(-c3cn(C)nc3C)sc12] | (S)-(1-methylpyrrolidin-2- yl)methyl (5-(2-(1,3- dimethyl-1H-pyrazol-4- yl)pyrazolo[5,1-b]thiazole-7- carboxamido)-6- methylpyridin-3-yl)carbamate |

In some aspects, the compounds according to formula (I) or formula (Jo) are those that have an IC₅₀<20 nM in a PDGFR cellular assay such as, for example, that described in the Experimental section below.

In some embodiments, the compounds according to formula (I) or formula (I₀) are those that have an IC₅₀<5 nM in a PDGFR cellular assay such as, for example, that described in the Experimental section below.

In some aspects, the compound of the disclosure is 2-(6,7-dihydro-5H-pyrazolo[5,1-b][1,3]oxazin-3-yl)-N-(5-(2-(3,3-dimethylazetidin-1-yl)acetamido)-2-methylpyridin-3-yl)pyrazolo[5,1-b]thiazole-7-carboxamide; N-(5-(3-(2,2-dimethylpyrrolidin-1-yl)propanamido)-2-methyl pyridin-3-yl)-2-(1-(2-methoxyethyl)-1H-pyrazol-4-yl)pyrazolo[5,1-b]thiazole-7-carboxamide; N-(5-(3-(2-azabicyclo[2.2.2]octan-2-yl)propanamido)-2-methyl pyridin-3-yl)-2-(1-methyl-1H-pyrazol-4-yl)pyrazolo[5,1-b]thiazole-7-carboxamide; N-(5-((2-(1-azaspiro[3.3]heptan-1-yl)ethyl)carbamoyl)-2-methylpyridin-3-yl)-2-(1,5-dimethyl-1H-pyrazol-4-yl)pyrazolo[5,1-b]thiazole-7-carboxamide; 2-(1,5-dimethyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide; 2-(1,3-dimethyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide; N-(5-((2-(2-azabicyclo[2.2.2]octan-2-yl)ethyl)carbamoyl)-2-methylpyridin-3-yl)-2-(1,3-dimethyl-1H-pyrazol-4-yl)pyrazolo[5,1-b]thiazole-7-carboxamide; N-(5-((2-(5-azaspiro[3.4]octan-5-yl)ethyl)carbamoyl)-2-methyl-pyridin-3-yl)-2-(1,5-dimethyl-1H-pyrazol-4-yl)pyrazolo[5,1-b]thiazole-7-carboxamide; N-(5-((2-(5-azaspiro[3.4]octan-5-yl)ethyl)carbamoyl)-2-methylpyridin-3-yl)-2-(1,3-dimethyl-1H-pyrazol-4-yl)pyrazolo[5,1-b]thiazole-7-carboxamide; N-(5-(2-(3,3-dimethylazetidin-1-yl)acetamido)-2-methylpyridin-3-yl)-2-(1-(oxetan-3-yl)-1H-pyrazol-4-yl)pyrazolo[5,1-b]thiazole-7-carboxamide; N-(5-(2-(2,2-dimethylpyrrolidin-1-yl)acetamido)-2-methylpyridin-3-yl)-2-(1-(1-hydroxy-2-methylpropan-2-yl)-1H-pyrazol-4-yl)pyrazolo[5,1-b]thiazole-7-carboxamide; 2-(6,7-dihydro-4H-pyrazolo[5,1-c][1,4]oxazin-3-yl)-N-(5-(2-(2,2-dimethylpyrrolidin-1-yl)acetamido)-2-methylpyridin-3-yl)pyrazolo[5,1-b]thiazole-7-carboxamide; N-(5-((2-(5-azaspiro[3.4]octan-5-yl)ethyl)carbamoyl)-2-methylpyridin-3-yl)-2-(5,6-dihydro-4H-pyrrolo[1,2-b]pyrazol-3-yl)pyrazolo[5,1-b]thiazole-7-carboxamide; N-(5-(2-(cyclobutylamino)acetamido)-2-methylpyridin-3-yl)-2-(5,6-dihydro-4H-pyrrolo[1,2-b]pyrazol-3-yl)pyrazolo[5,1-b]thiazole-7-carboxamide; N-(5-((2-(5-azaspiro[3.4]octan-5-yl)ethyl)carbamoyl)-2-methylpyridin-3-yl)-2-(6,7-dihydro-4H-pyrazolo[5,1-c][1,4]oxazin-3-yl)pyrazolo[5,1-b]thiazole-7-carboxamide; (S)-2-(6,7-dihydro-4H-pyrazolo[5,1-c][1,4]oxazin-3-yl)-N-(2-methyl-5-(2-(2-methylpyrrolidin-1-yl)acetamido)pyridin-3-yl)pyrazolo[5,1-b]thiazole-7-carboxamide; (S)—N-(5-(2-(1-isopropylpyrrolidin-3-yl)acetamido)-2-methylpyridin-3-yl)-2-(1-(2-methoxyethyl)-1H-pyrazol-4-yl)pyrazolo[5,1-b]thiazole-7-carboxamide; or 2-(5,6-dihydro-4H-pyrrolo[1,2-b]pyrazol-3-yl)-N-(5-((2-(3,3-dimethylazetidin-1-yl)ethyl)carbamoyl)-2-methylpyridin-3-yl)pyrazolo[5,1-b]thiazole-7-carboxamide; or a pharmaceutically salt of one of these compounds.

In some aspects, the compound of the disclosure is N-(5-((2-(1-azaspiro[3.3]heptan-1-yl)ethyl)carbamoyl)-2-methylpyridin-3-yl)-2-(1,5-dimethyl-1H-pyrazol-4-yl)pyrazolo[5,1-b]thiazole-7-carboxamide; 2-(1,5-dimethyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide; 2-(1,3-dimethyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide; N-(5-((2-(2-azabicyclo[2.2.2]octan-2-yl)ethyl)carbamoyl)-2-methylpyridin-3-yl)-2-(1,3-dimethyl-1H-pyrazol-4-yl)pyrazolo[5,1-b]thiazole-7-carboxamide; N-(5-((2-(5-azaspiro[3.4]octan-5-yl)ethyl)carbamoyl)-2-methyl-pyridin-3-yl)-2-(1,5-dimethyl-1H-pyrazol-4-yl)pyrazolo[5,1-b]thiazole-7-carboxamide; N-(5-((2-(5-azaspiro[3.4]octan-5-yl)ethyl)carbamoyl)-2-methylpyridin-3-yl)-2-(1,3-dimethyl-1H-pyrazol-4-yl)pyrazolo[5,1-b]thiazole-7-carboxamide; N-(5-(2-(3,3-dimethylazetidin-1-yl)acetamido)-2-methylpyridin-3-yl)-2-(1-(oxetan-3-yl)-1H-pyrazol-4-yl)pyrazolo[5,1-b]thiazole-7-carboxamide; N-(5-(2-(2,2-dimethylpyrrolidin-1-yl)acetamido)-2-methylpyridin-3-yl)-2-(1-(1-hydroxy-2-methylpropan-2-yl)-1H-pyrazol-4-yl)pyrazolo[5,1-b]thiazole-7-carboxamide; 2-(6,7-dihydro-4H-pyrazolo[5,1-c][1,4]oxazin-3-yl)-N-(5-(2-(2,2-dimethylpyrrolidin-1-yl)acetamido)-2-methylpyridin-3-yl)pyrazolo[5,1-b]thiazole-7-carboxamide; N-(5-((2-(5-azaspiro[3.4]octan-5-yl)ethyl)carbamoyl)-2-methylpyridin-3-yl)-2-(5,6-dihydro-4H-pyrrolo[1,2-b]pyrazol-3-yl)pyrazolo[5,1-b]thiazole-7-carboxamide; N-(5-(2-(cyclobutylamino)acetamido)-2-methylpyridin-3-yl)-2-(5,6-dihydro-4H-pyrrolo[1,2-b]pyrazol-3-yl)pyrazolo[5,1-b]thiazole-7-carboxamide; N-(5-((2-(5-azaspiro[3.4]octan-5-yl)ethyl)carbamoyl)-2-methylpyridin-3-yl)-2-(6,7-dihydro-4H-pyrazolo[5,1-c][1,4]oxazin-3-yl)pyrazolo[5,1-b]thiazole-7-carboxamide; (S)-2-(6,7-dihydro-4H-pyrazolo[5,1-c][1,4]oxazin-3-yl)-N-(2-methyl-5-(2-(2-methylpyrrolidin-1-yl)acetamido)pyridin-3-yl)pyrazolo[5,1-b]thiazole-7-carboxamide; (S)—N-(5-(2-(1-isopropylpyrrolidin-3-yl)acetamido)-2-methylpyridin-3-yl)-2-(1-(2-methoxyethyl)-1H-pyrazol-4-yl)pyrazolo[5,1-b]thiazole-7-carboxamide; or 2-(5,6-dihydro-4H-pyrrolo[1,2-b]pyrazol-3-yl)-N-(5-((2-(3,3-dimethylazetidin-1-yl)ethyl)carbamoyl)-2-methylpyridin-3-yl)pyrazolo[5,1-b]thiazole-7-carboxamide; or a pharmaceutically salt of one of these compounds.

References to formula (I) herein encompass any subgenera of those formula disclosed herein (e.g., formula (IA), (IA-1), (IA-2), and (IA-3), (IB), (IB-1), (IC), (IC-1), (IC-2)).

References to formula (Io) herein encompass any subgenera of those formula disclosed herein (e.g., formula (IAo), (IAo-1), (IAo-2), and (IAo-3), (IBo), (IBo-1), (IDo), (IEo), (IFo)).

Stereoisomers of compounds of formula (I) or compounds of formula (Io) are also contemplated by the present disclosure. Thus, the disclosure encompasses all stereoisomers and constitutional isomers of any compound disclosed or claimed herein, including all enantiomers and diastereomers.

Pharmaceutically acceptable salts and solvates of the compounds of formula (I) or the compounds of formula (Io) are also within the scope of the disclosure.

Isotopic variants of the compounds of formula (I) or the compounds of formula (Io) are also contemplated by the present disclosure.

### Pharmaceutical Compositions and Methods of Administration

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. In some embodiments, the pharmaceutical compositions contain a compound of the present disclosure or a pharmaceutically acceptable salt thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 30, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

In some embodiments, the compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

Unless otherwise noted, the amounts of the compounds described herein are set forth on a free base basis. That is, the amounts indicate that amount of the compound administered, exclusive of, for example, solvent (such as in solvates) or counterions (such as in pharmaceutically acceptable salts).

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof, lysophospholipids and derivatives thereof, carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-lOoleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, F-caprolactone and isomers thereof, S-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%), 100%, or up to about 200%>by weight, based on the combined weight of the drug, and other excipients. If desired, very small amounts of solubilizer may also be used, such as 5%>, 2%>, 1%) or even less. Typically, the solubilizer may be present in an amount of about 1%>to about 100%, more typically about 5%>to about 25%>by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical compositions for topical (e.g. transdermal) delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

Compositions for inhalation may be delivered as a dry powder (either alone, as a mixture, for example a dry blend with lactose, or a mixed component particle, for example with phospholipids) from a dry powder inhaler or as an aerosol spray from a pressurised container, pump, spray, atomizer or nebuliser, with or without the use of a suitable propellant. Such devices are referred to in, for example, WO2013030802.

Where the inhalable form of the active ingredient is an aerosol composition, the inhalation device may be an aerosol vial provided with a valve adapted to deliver a metered dose, i.e. a metered dose inhaler. Where the inhalable form of the active ingredient is a nebulizable aqueous, organic or aqueous/organic dispersion, the inhalation device may be a nebulizer, such as an airjet nebulizer, or an ultrasonic nebulizer, or a hand-held nebulizer, sometimes referred to as a soft mist or soft spray inhaler, or a mechanical device which allows much smaller nebulized volumes than conventional nebulizers. Such devices are referred to in, for example, WO2013030802.

Where the inhalable form of the active ingredient is the finely divided particulate form, the inhalation device may be, for example, a dry powder inhalation device adapted to deliver dry powder from a capsule or blister containing a dry powder comprising a dosage unit or a multidose dry powder inhalation (MDPI) device adapted to deliver dry powder comprising a dosage unit upon actuation. The dry powder composition preferably contains a diluent or carrier, such as lactose, and a compound that helps to protect against product performance deterioration due to moisture e.g. magnesium stearate. Dry powder inhalation devices are referred to in, for example, WO2013030802

Thus, in some embodiments, the invention also includes (A) a compound of the invention, or a pharmaceutically acceptable salt thereof, in inhalable form; (B) an inhalable medicament comprising the compound in inhalable form together with a pharmaceutically acceptable carrier in inhalable form; (C) a pharmaceutical product comprising such a compound in inhalable form in association with an inhalation device; and (D) an inhalation device containing such a compound in inhalable form.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intraarterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; U.S. Pat. Nos. 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

The disclosure also relates to methods of using the compounds described herein to treat in a subject in need thereof, a disease or disorder in which PDGFR signaling is implicated. These methods are accomplished by administering to the subject a compound of the disclosure in an amount effective to treat the disease or disorder.

In some aspects, the disclosure is directed to methods of using the compounds described herein to treat a disease or disorder in a subject in need thereof, wherein the disease or disorder is pulmonary hypertension (PH).

In some embodiments, the pulmonary hypertension is pulmonary arterial hypertension (PAH) (WHO PH Group 1); PH secondary to heart failure (WHO PH Group 2); PH secondary to lung diseases and/or hypoxia (WHO PH Group 3); PH due to pulmonary artery obstruction (WHO Group 4); or PH due to unknown or rare diseases (WHO PH Group 5).

In some embodiments, the PAH (WHO PH Group 1) is idiopathic PAH, PAH with vasoreactivity, heritable PAH, drugs and toxins-induced PAH, PAH associated with connective tissue disease, PAH associated with HIV infection, PAH associated with portal hypertension, PAH associated with congenital heart disease, PAH associated with schistosomiasis, PAH in long-term responders to calcium channel blockers, PAH with overt signs of venous/capillaries involvement; persistent PH of the Newborn syndrome; or systemic sclerosis-associated PAH (SSc-PAH).

In some embodiments, the PAH secondary to heart failure (WHO PH Group 2) is PH due to heart failure with preserved ejection fraction, PH due to heart failure with reduced ejection fraction, valvular heart disease, or congenital post-capillary obstructive lesions.

In some embodiments, the PH secondary to lung diseases and/or hypoxia (WHO PH Group 3) is PH due to obstructive lung disease, PH due to restrictive lung disease, PH due to other lung diseases with mixed restrictive/obstructive pattern, PH due to hypoxia without lung disease, PH due to developmental lung disorders.

In some embodiments, the PH due to obstructive lung disease is PH due to chronic obstructive pulmonary disease (COPD).

In some embodiments, the PH due to restrictive lung disease is PH due to interstitial lung diseases (ILDs).

In some embodiments, the PH due to interstitial lung diseases (ILDs) is PH due to idiopathic pulmonary fibrosis (IPF).

In some embodiments, the PH due to pulmonary artery obstruction (WHO Group 4) is chronic thromboembolic PH (CTEPH) or PH due to other pulmonary artery obstructions.

In some embodiments, the PH due to unknown or rare diseases (WHO PH Group 5) is PH due to hematologic disorders, PH due to systemic disorders, PH due to other disorders, or PH due to complex congenital heart disease.

In some aspects, the disclosure is directed to methods of using the compounds described herein to treat a disease or disorder in a subject in need thereof, wherein the disease or disorder is a respiratory disease.

In some embodiments, the respiratory disease is asthma.

In some aspects, the disclosure is directed to methods of using the compounds described herein to treat a disease or disorder in a subject in need thereof, wherein the disease or disorder is a fibrotic disease.

In some embodiments, the fibrotic disease is pulmonary fibrosis, cardiac fibrosis or liver fibrosis.

In some embodiments, the fibrotic disease is pulmonary fibrosis.

In some embodiments, the pulmonary fibrosis is an interstitial lung disease.

In some embodiments, the interstitial lung disease is idiopathic pulmonary fibrosis.

In some embodiments, the interstitial lung disease is rheumatoid arthritis-associated interstitial lung disease.

In some embodiments, the interstitial lung disease is systemic sclerosis-associated interstitial lung disease.

In some embodiments, the interstitial lung disease is connective tissue disease-associated interstitial lung disease.

In some embodiments, the interstitial lung disease is nonspecific interstitial pneumonia.

In some embodiments, the interstitial lung disease is unclassifiable interstitial lung disease.

In some embodiments, the interstitial lung disease is hypersensitivity pneumonitis.

In some embodiments, the interstitial lung disease is sarcoidosis.

In some embodiments, the interstitial lung disease is non-idiopathic pulmonary fibrosis interstitial lung disease.

In some aspects, the disclosure is directed to methods of using the compounds described herein to treat a disease or disorder in a subject in need thereof, wherein the disease or disorder is a dermatological disease.

In some embodiments, the dermatological disease or disorder is atopic dermatitis, scleroderma, or urticaria.

In some aspects, the disclosure is directed to methods of using the compounds described herein to treat a disease or disorder in a subject in need thereof, wherein the disease or disorder is an inflammatory disease or disorder.

In some embodiments, the inflammatory disease or disorder is allergic rhinitis, irritable bowel syndrome (IBS); or inflammatory bowel disease (IBD).

In some aspects, the disclosure is directed to methods of using the compounds described herein to treat a disease or disorder in a subject in need thereof, wherein the disease or disorder is an autoimmune disorder.

In some aspects, the disclosure is directed to methods of using the compounds described herein to treat a disease or disorder in a subject in need thereof, wherein the disease or disorder is a metabolic disease.

In some aspects, the disclosure is directed to methods of using the compounds described herein to treat a disease or disorder in a subject in need thereof, wherein the disease or disorder is vascular restenosis; age-related macular degeneration (AMD); irritable bowel syndrome (IBS); inflammatory bowel disease (IBD); obesity-cell related diseases; type I diabetes or type II diabetes.

In other embodiments, the disclosure is directed to methods of using the compounds described herein to treat a disease or disorder in a subject in need thereof, wherein the disease or disorder is pulmonary arterial hypertension (PAH).

In other embodiments, the disclosure is directed to methods of using the compounds described herein to treat a disease or disorder in a subject in need thereof, wherein the disease or disorder is PH secondary to heart failure (WHO PH Group 2).

In other embodiments, the disclosure is directed to methods of using the compounds described herein to treat a disease or disorder in a subject in need thereof, wherein the disease or disorder is PH due to heart failure with preserved ejection fraction.

In other embodiments, the disclosure is directed to methods of using the compounds described herein to treat a disease or disorder in a subject in need thereof, wherein the disease or disorder is PH due to heart failure with reduced ejection fraction.

In other embodiments, the disclosure is directed to methods of using the compounds described herein to treat a disease or disorder in a subject in need thereof, wherein the disease or disorder is valvular heart disease.

In other embodiments, the disclosure is directed to methods of using the compounds described herein to treat a disease or disorder in a subject in need thereof, wherein the disease or disorder is congenital post-capillary obstructive lesions.

In other embodiments, the disclosure is directed to methods of using the compounds described herein to treat a disease or disorder in a subject in need thereof, wherein the disease or disorder is PH secondary to lung diseases and/or hypoxia (WHO PH Group 3).

In other embodiments, the disclosure is directed to methods of using the compounds described herein to treat a disease or disorder in a subject in need thereof, wherein the disease or disorder is PH due to pulmonary artery obstruction (WHO Group 4).

In other embodiments, the disclosure is directed to methods of using the compounds described herein to treat a disease or disorder in a subject in need thereof, wherein the disease or disorder is chronic thromboembolic PH (CTEPH).

In other embodiments, the disclosure is directed to methods of using the compounds described herein to treat a disease or disorder in a subject in need thereof, wherein the disease or disorder is PH due to unknown or rare diseases (WHO PH Group 5).

In other embodiments, the disclosure is directed to methods of using the compounds described herein to treat a disease or disorder in a subject in need thereof, wherein the disease or disorder is idiopathic PAH.

In other embodiments, the disclosure is directed to methods of using the compounds described herein to treat a disease or disorder in a subject in need thereof, wherein the disease or disorder is PAH associated with connective tissue disease.

In other embodiments, the disclosure is directed to methods of using the compounds described herein to treat a disease or disorder in a subject in need thereof, wherein the disease or disorder is systemic sclerosis-associated PAH (SSc-PAH).

In other embodiments, the disclosure is directed to methods of using the compounds described herein to treat a disease or disorder in a subject in need thereof, wherein the disease or disorder is PH secondary to interstitial lung diseases (ILDs).

In other embodiments, the disclosure is directed to methods of using the compounds described herein to treat a disease or disorder in a subject in need thereof, wherein the disease or disorder is PH secondary to chronic obstructive pulmonary disease (COPD).

In other embodiments, the disclosure is directed to methods of using the compounds described herein to treat a disease or disorder in a subject in need thereof, wherein the disease or disorder is PH secondary to idiopathic pulmonary fibrosis (IPF).

In treatment methods according to the disclosure, an effective amount of a pharmaceutical agent according to the disclosure is administered to a subject suffering from or diagnosed as having such a disease or disorder. An “effective amount” means an amount or dose sufficient to generally bring about the desired therapeutic benefit in patients in need of such treatment for the designated disease or disorder. Effective amounts or doses of the compounds of the present disclosure may be ascertained by routine methods such as modeling, dose escalation studies or clinical trials, and by taking into consideration routine factors, e.g., the mode or route of administration or drug delivery, the pharmacokinetics of the compound, the severity and course of the disease or disorder, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

In addition, the compounds of the disclosure may be used in combination with additional active ingredients in the treatment of the above diseases or disorders. The additional active ingredients may be coadministered separately with a compound of the disclosure or included with such an agent in a pharmaceutical composition according to the disclosure. The combination may serve to increase efficacy (e.g., by including in the combination a compound potentiating the potency or effectiveness of an active agent according to the disclosure), decrease one or more side effects, or decrease the required dose of the active agent according to the disclosure.

Compounds of formula (Io) and formula (I) in the present invention can be synthesized in accordance with general synthetic methods familiar to those who are skilled in the art. The following reaction schemes are only meant to represent examples of the invention and are in no way meant to be a limit of the invention.

The schemes below schemes below that illustrate synthesis of compounds of formula (I) and subgenera thereof can also be used to prepare some compounds of formula (I₀) and subgenera thereof.

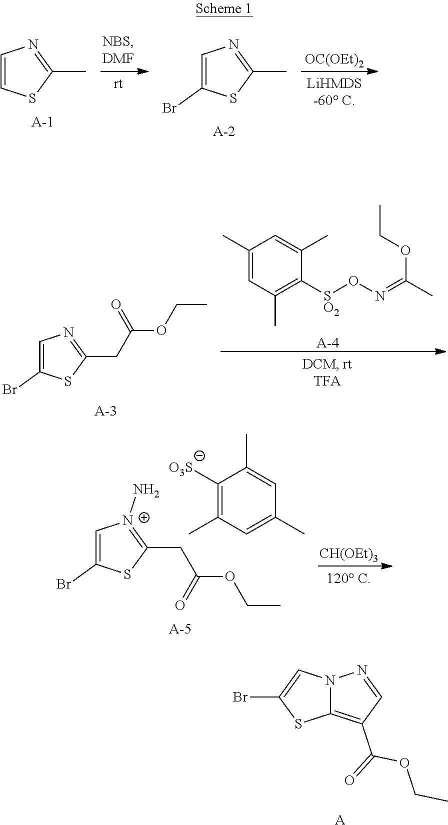

Scheme 1 illustrated the synthesis of key intermediate A. 2-methylthiazole (A-1) treated with NBS in DMF at room temperature to give 5-bromo-2-methylthiazole (A-2), 5-bromo-2methylthiazole was then reacted with LiHMDS and diethyl carbonate in THE yielded ethyl 2-(5-bromothiazol-2-yl)acetate (A-2), subsequently treated with ethyl (Z)—N-((mesitylsulfonyl)oxy)acetimidate (A-4) and TFA in dichloromethane to give A-5, reacted A-5 with triethyl orthoformate resulted ethyl 2-bromopyrazolo[5,1-b]thiazole-7-carboxylate (A).

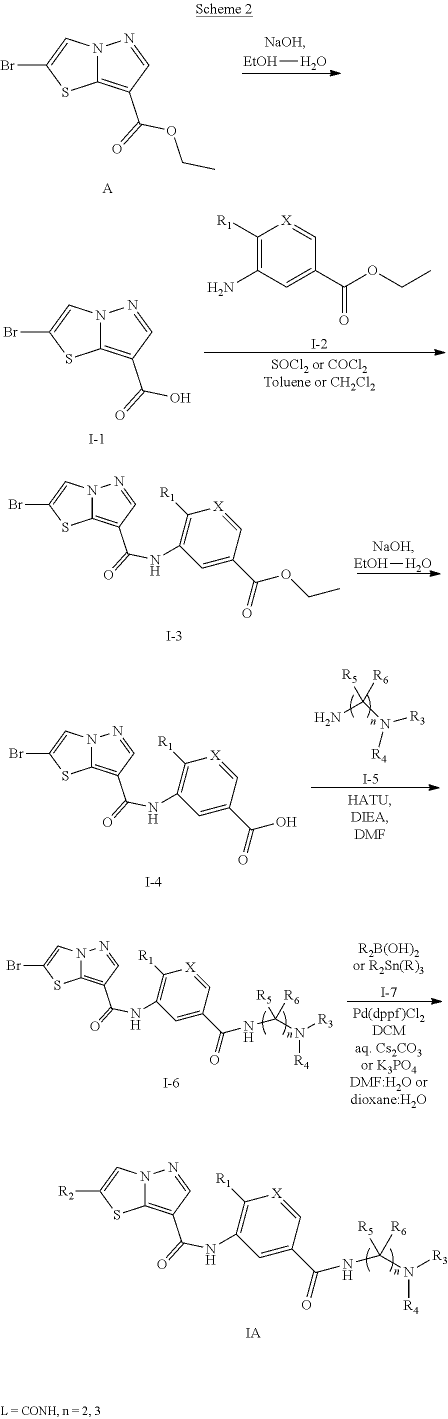

Scheme 2 show the synthesis of Formula IA while L=CONH, n=2, 3. Ethyl 2-bromopyrazolo[5,1-b]thiazole-7-carboxylate (A) hydrolyzed to 2-bromopyrazolo[5,1-b]thiazole-7-carboxylic acid (I-1) under a base such as NaOH in a solvent such as ethanol-water, 2-bromopyrazolo[5,1-b]thiazole-7-carboxylic acid (I-1) then converted into acid chloride with SOCl₂ or oxalyl dichloride in a solvent such as toluene or methylene chloride, the acid chloride reacted with amine (I-2) and a base such as DIEA or pyridine in a solvent such as methylene chloride or pyridine to give ester compound (I-3), ester compound (I-3) hydrolyzed to acid compound (I-4) under a base such as NaOH in a solvent such as ethanol-water, then treated with amine (I-5), a coupling reagent such as HATU, a base such as DIEA in a solvent such as DMF to produce compound (I-6), cross coupling compound (I-6) with a coupling reagent such a boronic acid or a boronate or a stannyl reagent (I-7) with a catalyst such as Pd(dppf)Cl₂ DCM, a base such as Cs₂CO₃ or K₃PO₄ in a solvent such as DMF-water or dioxane-water to give Formula IA compound while L=CONH, n=2, 3.

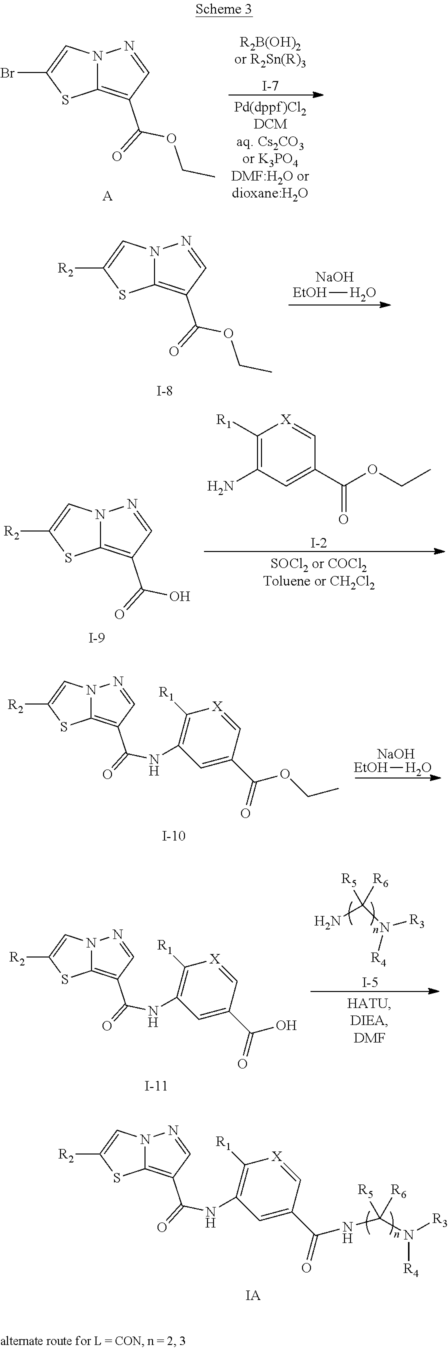

Scheme 3 show the alternative synthesis of Formula IA while L=CONH, n=2, 3. Ethyl 2-bromopyrazolo[5,1-b]thiazole-7-carboxylate (A) was coupled with a coupling reagent such a boronic acid or a boronate or a stannyl reagent (I-7) with a catalyst such as Pd(dppf)Cl2 DCM, a base such as Cs₂CO₃ or K₃PO₄ in a solvent such as DMF-water or dioxane-water to give ester (I-8), ester (I-8) then hydrolyzed to acid (I-9) under a base such as NaOH in a solvent such as ethanol-water, the acid (I-9) then converted into acid chloride with SOCl₂ or oxalyl dichloride in a solvent such as toluene or methylene chloride, the acid chloride reacted with amine (I-2) and a base such as DIEA or pyridine in a solvent such as methylene chloride or pyridine to give ester compound (I-10), ester compound (I-10) hydrolyzed to acid compound (I-11) under a base such as NaOH in a solvent such as ethanol-water, then treated with amine (I-5), a coupling reagent such as HATU, a base such as DIEA in a solvent such as DMF to produce Formula TA compound while L=CONH, n=2, 3.

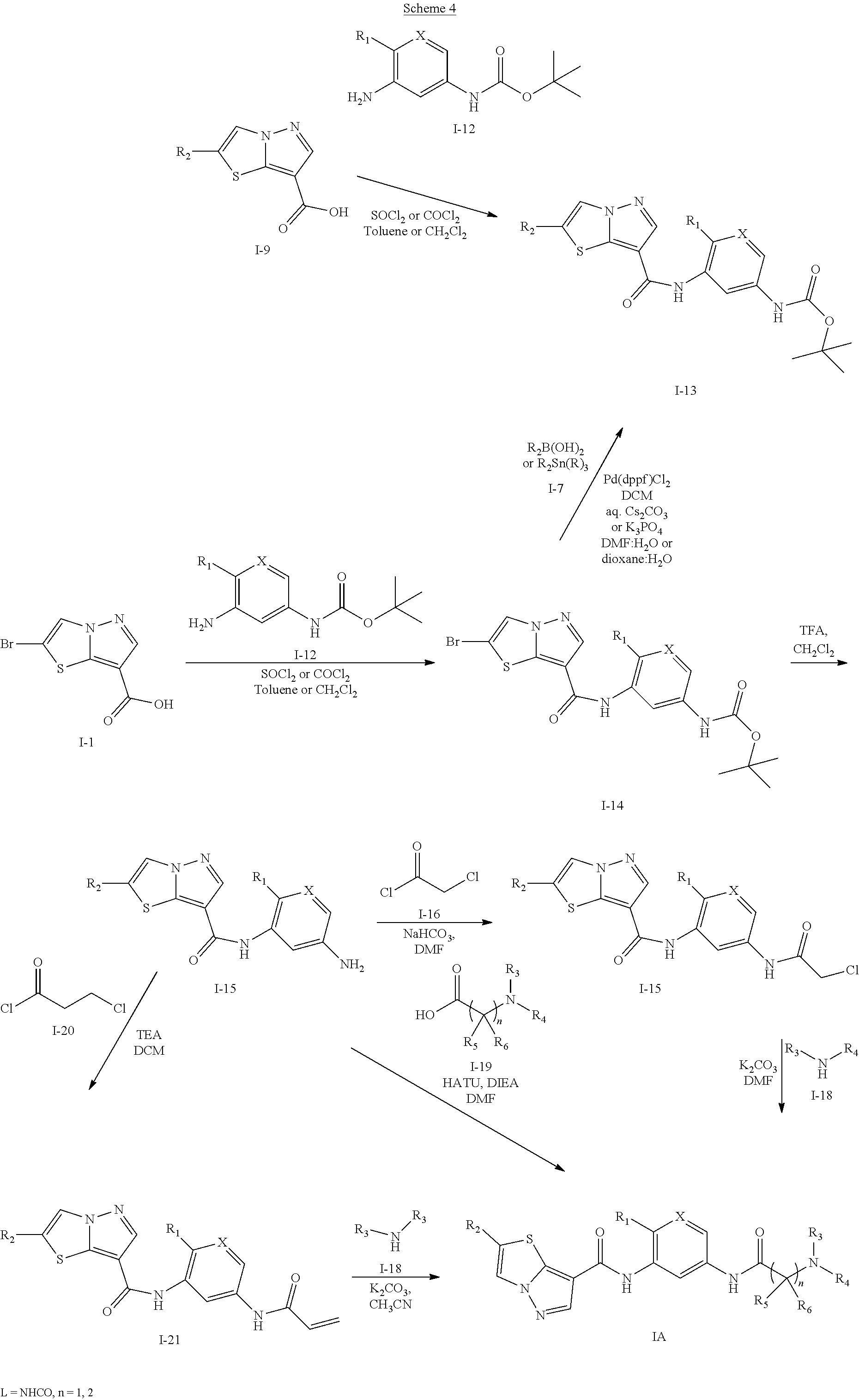

Scheme 4 show the synthesis of Formula IA while L=NHCO, n=1 or 2. Acid (I-9) was first converted into acid chloride with SOCl₂ or Oxalyl dichloride in a solvent such as toluene or methylene chloride, the acid chloride reacted with amine (I-12) and a base such as DIEA or pyridine in a solvent such as methylene chloride or pyridine to give a Boc-protected compound (I-13). Alternatively, acid (I-1) was converted into acid chloride with SOCl₂ or oxalyl dichloride in a solvent such as toluene or methylene chloride, the acid chloride reacted with amine (I-12) and a base such as DIEA or pyridine in a solvent such as methylene chloride or pyridine to give compound (I-14), compound (I-14) was then coupled with a coupling reagent such a boronic acid or a boronate or a stannyl reagent (I-7) with a catalyst such as Pd(dppf)Cl₂ DCM, a base such as Cs₂CO₃ or K₃PO₄ in a solvent such as DMF-water or dioxane-water to give a Boc-protected compound (I-13). Deprotecting Boc by treating compound (I-13) with acid such as TFA in a solvent such as methylene chloride followed by treatment with chloroacetyl chloride (I-16) and a base such as NaHCO₃ in a solvent such as DMF to give compound (I-17). Compound (I-17) was then reacted with amine (I-18) and a base such as K₂CO₃ in a solvent such as DMF to produce Formula I while L=NHCO, n=0 or 1. Alternatively, compound (I-15) treated with an acid (I-19), a coupling agent such as HATU, a base such as DIEA in a solvent such as DMF to yield Formula I compound while L=NHCO, n=1 or 2. Further alternatively, compound (I-15) treated with 3-chloropropanoyl chloride (I-20) and a base such as triethylamine in a solvent such as dichloromethane to give compound (I-21) which then reacted with amine (I-18) and a base such as K₂CO₃ in a solvent such as DMF to produce Formula IA while L=NHCO, n=0 or 1.

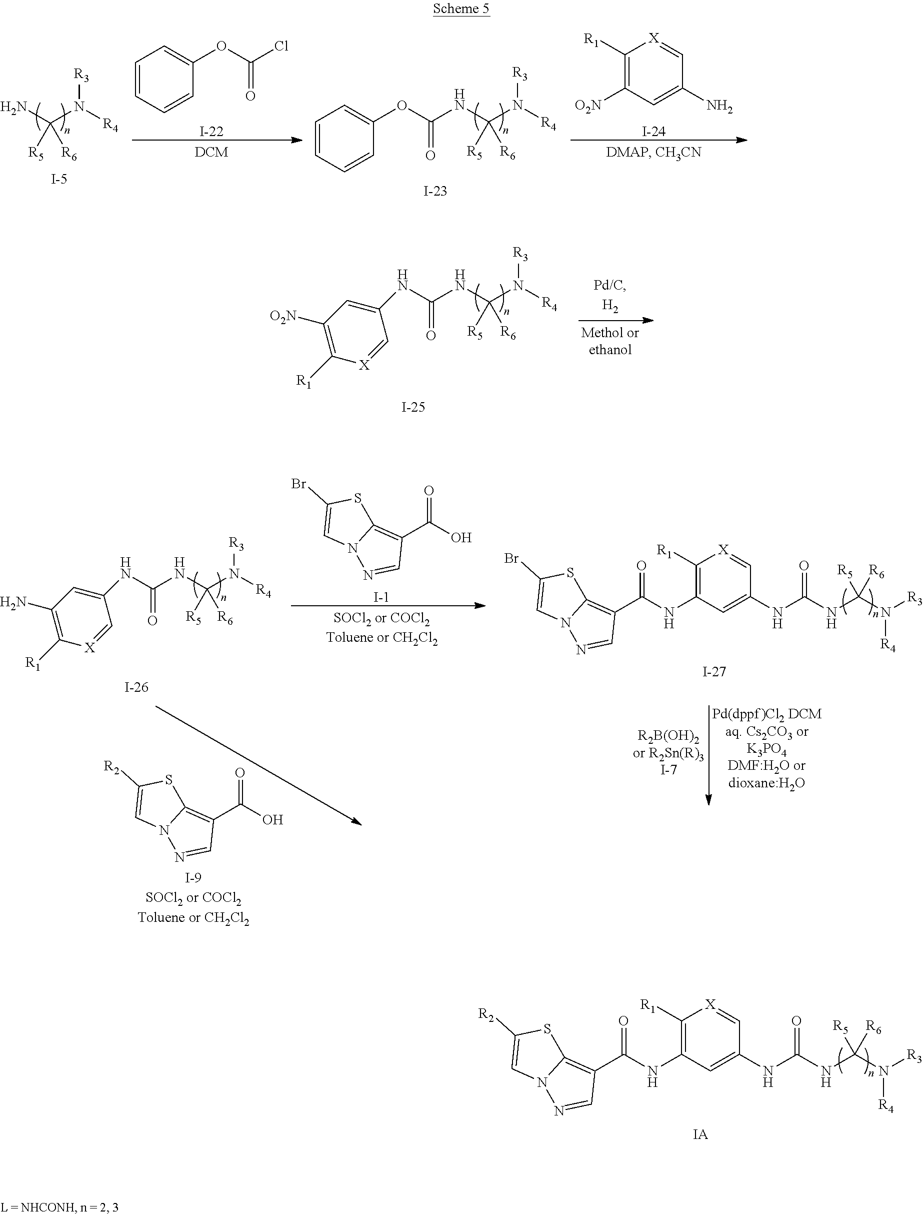

Scheme 5 show the synthesis of Formula IA while L=NHCONH, n=2 or 3. Amine (I-5) treated with phenyl carbonochloridate (I-22) in a solvent such as dichloromethane to give compound (I-23), compound (I-23) then reacted with amine (I-24) and a base such as DMAP in a solvent such as acetyl nitrile to produce nitro compound (I-25) which then reduced to amine (I-26) through hydrogenation under a catalyst such as Pd/C in a solvent such as methanol or ethanol. Acid (I-1) was first converted into acid chloride with SOCl₂ or Oxalyl dichloride in a solvent such as toluene or methylene chloride, the acid chloride reacted with compound (I-26) and a base such as DIEA or pyridine in a solvent such as methylene chloride or pyridine to give compound (I-27), compound (I-27) was then coupled with a coupling reagent such a boronic acid or a boronate or a stannyl reagent (I-7) with a catalyst such as Pd(dppf)Cl₂ DCM, a base such as Cs₂CO₃ or K₃PO₄ in a solvent such as DMF-water or dioxane-water to yield Formula IA compound while L=NHCONH, n=2 or 3. Alternatively, acid (I-9) was converted into acid chloride with SOCl₂ or oxalyl dichloride in a solvent such as toluene or methylene chloride, the acid chloride reacted with compound (I-26) and base such as DIEA or pyridine in a solvent such as methylene chloride or pyridine to give Formula IA compound while L=NHCONH, n=2 or 3.

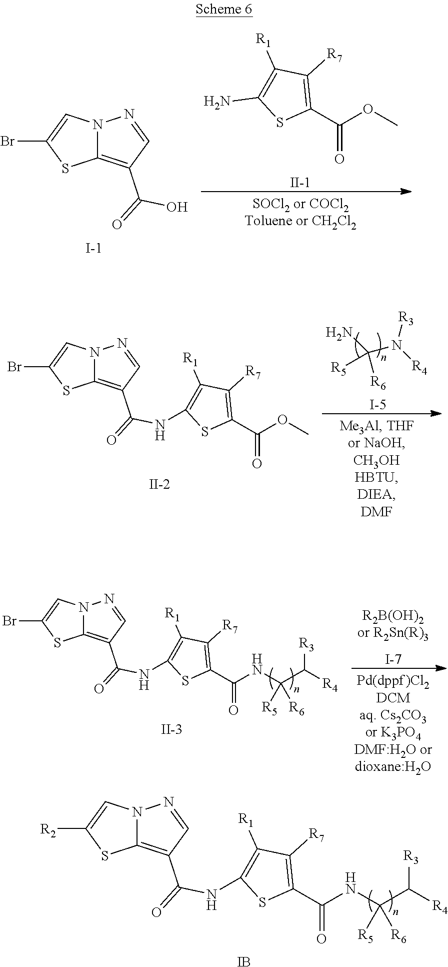

Scheme 6 show the synthesis of Formula IB, 2-bromopyrazolo[5,1-b]thiazole-7-carboxylic acid (I-1) then converted into acid chloride with SOCl₂ or oxalyl dichloride in a solvent such as toluene or methylene chloride, the acid chloride reacted with amine (II-1) and a base such as DIEA or pyridine in a solvent such as methylene chloride or pyridine to give ester compound (II-2), ester compound (II-2) then treated with amine (I-5) and Me₃Al in a solvent such as THF to produce compound (II-3), alternatively, ester compound (II-2) can be hydrolyzed to acid using a base such as NaOH in a solvent such as methanol or THE followed by coupling with amine (I-5) in the present of a coupling agent such as HATU, a base such as DIEA in a solvent DMF to give compound (II-3). Cross coupling compound (II-3) with a coupling reagent such a boronic acid or a boronate or a stannyl reagent (I-7) with a catalyst such as Pd(dppf)Cl₂ DCM, a base such as Cs₂CO₃ or K₃PO₄ in a solvent such as DMF-water or dioxane-water to give Formula IB compound.

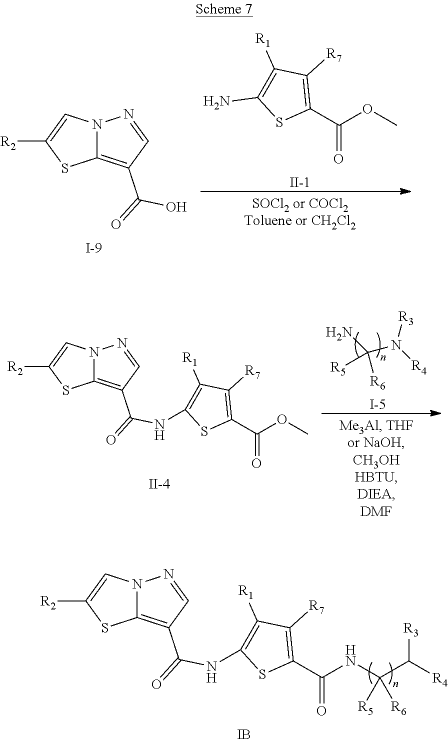

Scheme 7 show alternative synthesis of Formula IB, acid compound (I-9) then converted into acid chloride with SOCl₂ or oxalyl dichloride in a solvent such as toluene or methylene chloride, the acid chloride reacted with amine (II-1) and base such as DIEA or pyridine in a solvent such as methylene chloride or pyridine to give ester compound (II-4), ester compound (II-4) then treated with amine (I-5) and Me₃Al in a solvent such as THE to produce Formula IB compound. Alternatively, ester compound (II-2) can be hydrolyzed to acid using a base such as NaOH in a solvent such as methanol or THE followed by coupling with amine (I-5) in the present of a coupling agent such as HATU, a base such as DIEA in a solvent DMF to give Formula IB compound.

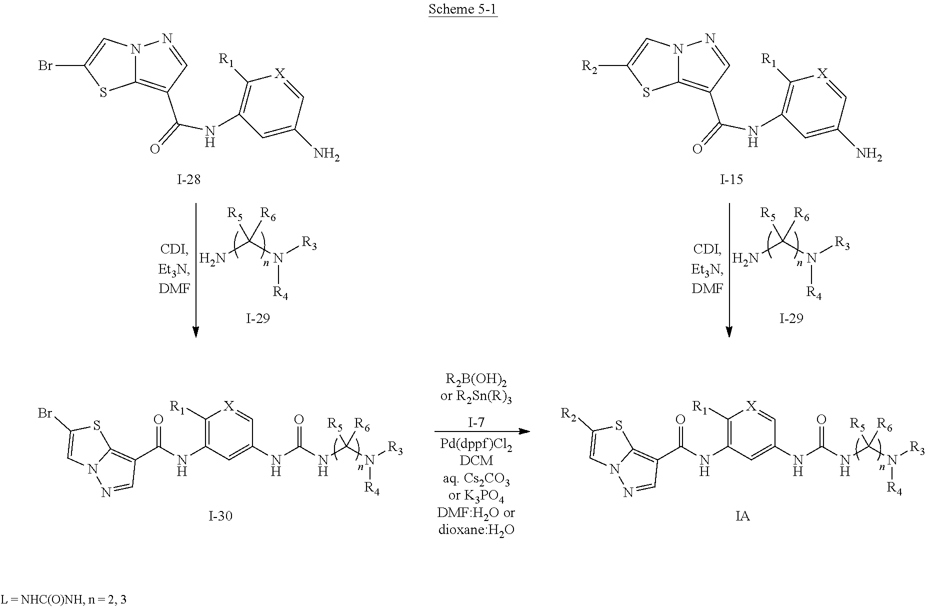

Scheme 5-1 shows the synthesis of Formula IA while L=NHC(O)NH, n=2 or 3. The compound (I-28) was first treated with 1,1′-carbonyldiimidazole (CDI), in the presence of a base such as Et₃N or DIEA, in a solvent such as DMF, and was then reacted with amine (I-29) to give urea compound (I-30). Compound (I-30) was then coupled with a coupling reagent such a boronic acid or a boronate or a stannyl reagent (I-7) with a catalyst such as Pd(dppf)Cl₂ DCM, a base such as Cs₂CO₃ or K₃PO₄ in a solvent such as DMF-water or dioxane-water to produce Formula IA while L=NHC(O)NH, n=2 or 3. Alternatively, compound (I-15) was first treated with 1,1′-carbonyldiimidazole (CDI), in the presence of a base such as Et₃N or DIEA, in a solvent such as DMF, and was then reacted with amine (I-29) to produce Formula IA while L=NHC(O)NH, n=2 or 3.

### General Scheme (Formula IA with C—N Linkage)

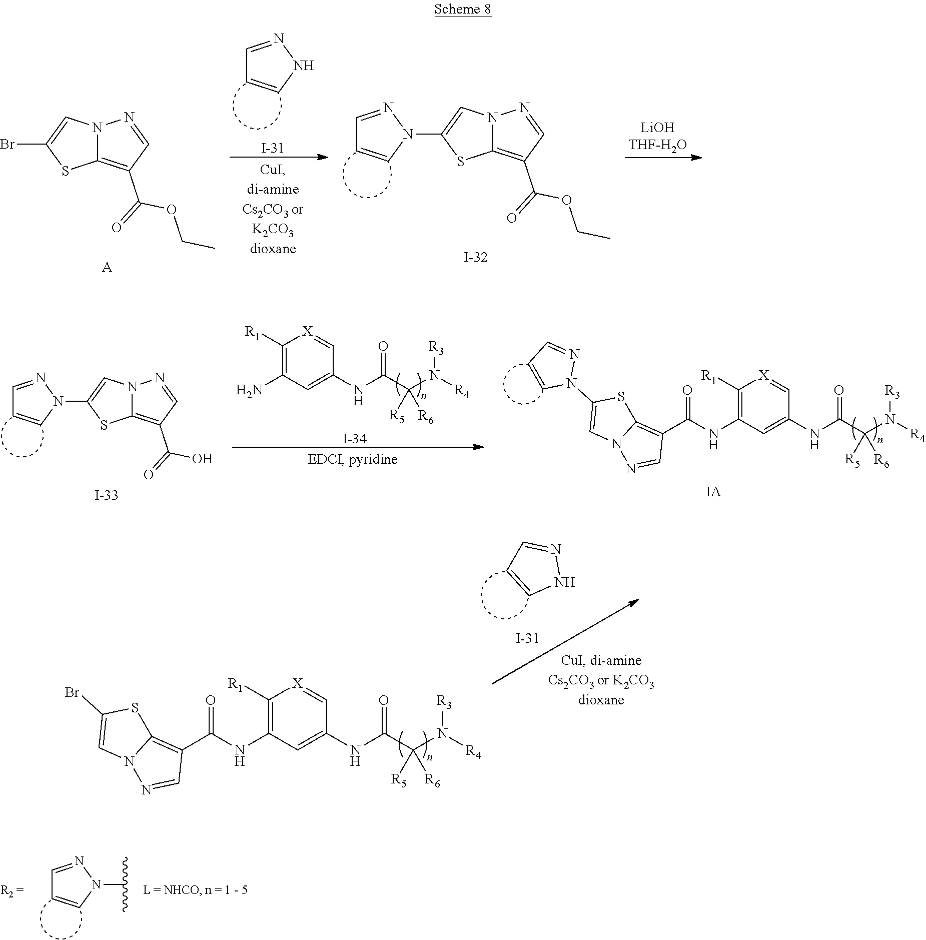

Scheme 8 shows the synthesis of Formula IA while

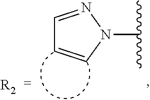

L=NHCO, n=1-5. Ethyl 2-bromopyrazolo[5,1-b]thiazole-7-carboxylate (A) was coupled with compound (I-31) with a catalyst such as CuI, in the presence of a di-amine, such as trans-N,N′-dimethylcyclohexane-1,2-diamine or N,N′-dimethylethane-1,2-diamine, a base such as Cs₂CO₃ or K₃PO₄ or K₂CO₃, in a solvent such as 1,4-dioxane to give compound (I-32). Compound (I-32) was hydrolyzed to acid (I-33) under a base such as LiOH or NaOH in a solvent such as THE-water or ethanol-water. Acid (I-33) was coupled with amine (I-34), in the presence of a coupling reagent such as EDCI, in a solvent such as pyridine to produce Formula IA while

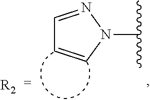

L=NHCO, n=1-5. Alternatively, compound (I-35) was coupled with compound (I-31) with a catalyst such as CuI, in the presence of a di-amine, such as trans-N,N′-dimethylcyclohexane-1,2-diamine or N,N′-dimethylethane-1,2-diamine, a base such as Cs₂CO₃ or K₃PO₄ or K₂CO₃, in a solvent such as 1,4-dioxane to produce Formula IA while

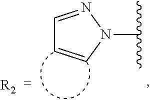

L=NHCO, n=1-5.

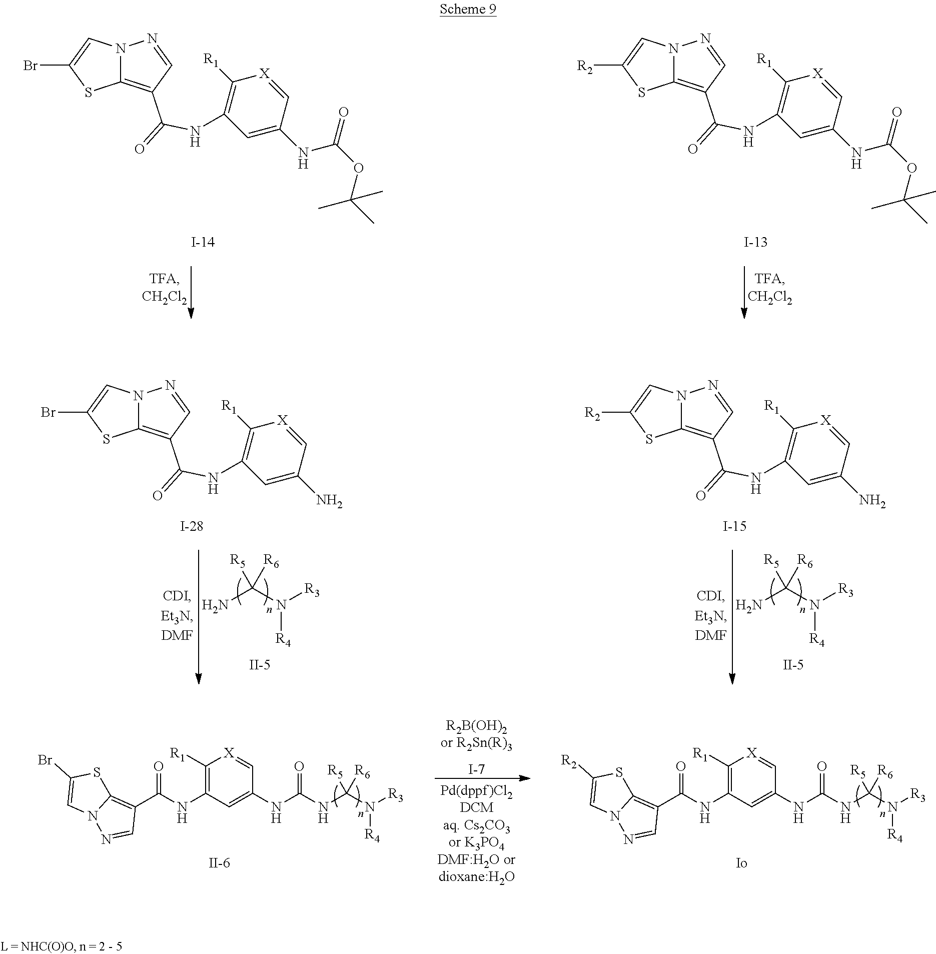

Scheme 9 shows the synthesis of Formula Io while L=NHC(O)O, n=2, 3, 4, or 5. The Boc-protected compound (I-14) was treated with an acid such as TFA in a solvent such as methylene chloride to give de-protected compound (I-28). The compound (I-28) was first treated with 1,1′-carbonyldiimidazole (CDI), in the presence of a base such as Et₃N or DIEA, in a solvent such as DMF, and was then reacted with alcohol (II-5) to give carbamate compound (II-6). Compound (II-6) was then coupled with a coupling reagent such a boronic acid or a boronate or a stannyl reagent (I-7) with a catalyst such as Pd(dppf)Cl₂ DCM, a base such as Cs₂CO₃ or K₃PO₄ in a solvent such as DMF-water or dioxane-water to produce Formula Io while L=NHC(O)O, n=2, 3, 4, or 5. Alternatively, compound (I-14) was coupled with a coupling reagent such a boronic acid or a boronate or a stannyl reagent (I-7) with a catalyst such as Pd(dppf)Cl₂ DCM, a base such as Cs₂CO₃ or K₃PO₄ in a solvent such as DMF-water or dioxane-water to give a Boc-protected compound (I-13). Deprotecting Boc by treating compound (I-13) with acid such as TFA in a solvent such as methylene chloride gave compound (I-15). Compound (I-15) was first treated with 1,1′-carbonyldiimidazole (CDI), in the presence of a base such as Et₃N or DIEA, in a solvent such as DMF, and was then reacted with alcohol (II-4) to produce Formula Io while L=NHC(O)O, n=2, 3, 4, or 5.

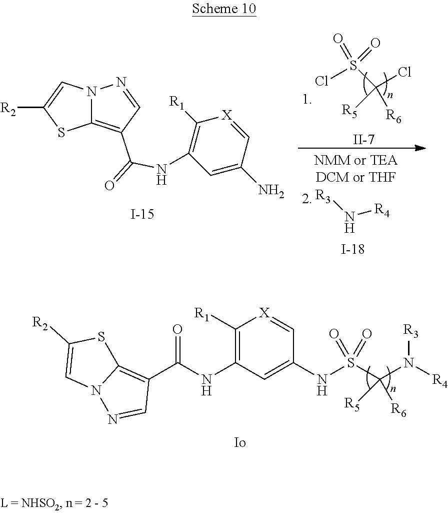

Scheme 10 shows the synthesis of compounds of Formula Io with L=NHSO₂, n=2-5. Amine I-15 is sulfonylated with a sulfonyl chloride (II-7) such as 2-chloroethylsulfonyl chloride or chloromethylsulfonyl chloride in the presence of base such as N-methylmorpholine or triethylamine in a solvent such as DCM or THF. The resulting crude chloride is reacted with amine I-18 to give compounds of Formula Io with L=NHSO₂, n=2-5.

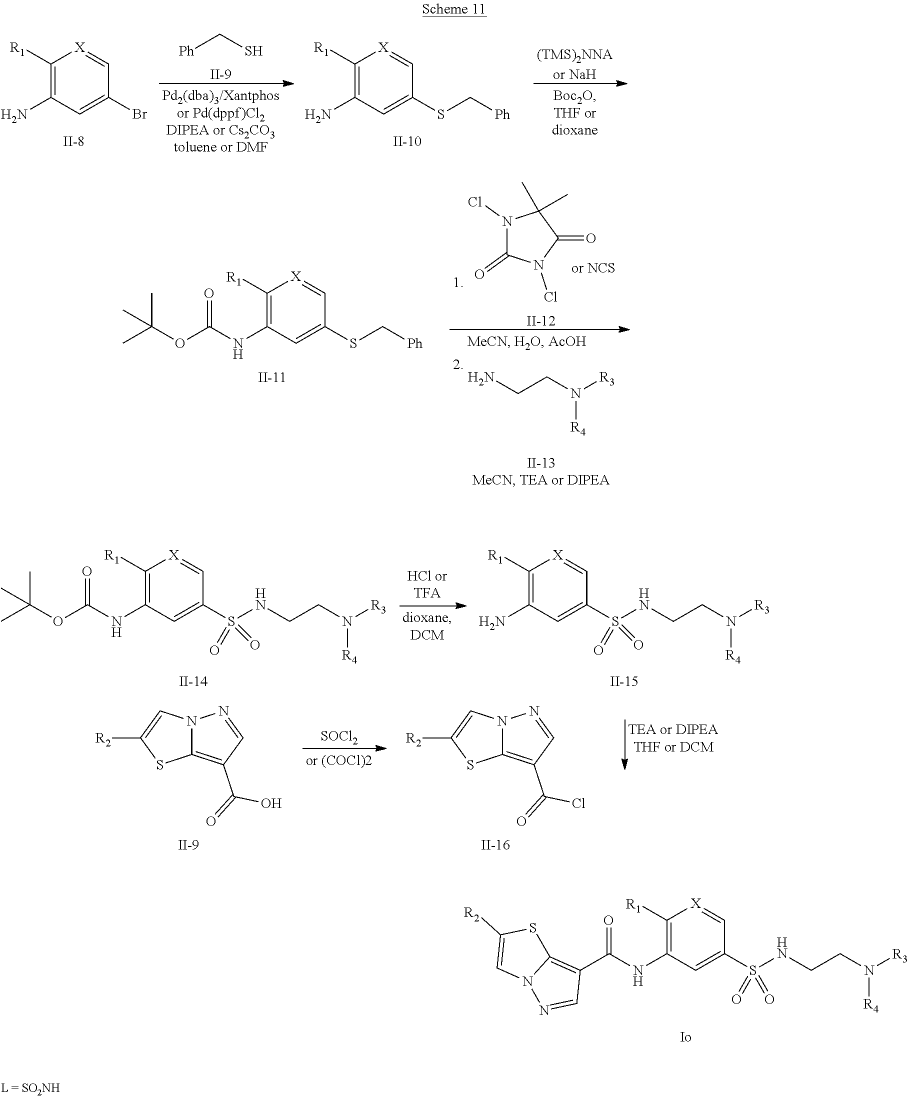

Scheme 11 shows the synthesis of compounds of Formula Io with L=SO₂NH. A bromide (II-8) such as 5-bromo-2-methylpyridin-3-amine was coupled with benzyl mercaptan (II-9) via a catalyst such as palladium tris(dibenzylideneacetone)dipalladium/Xantphos or [1,1′-bis(diphenylphosphino)ferrocene]dichloropalladium in the presence of a base such as diisopropylethyl amine or cesium carbonate in a solvent such as toluene or DMF to yield thioether II-10. Deprotonation of the aniline nitrogen with a base such as sodium bis(trimethylsilyl)amide or sodium hydride in a solvent such as THE or 1,4-dioxane followed by reaction with di-tert-butyl dicarbonate resulted in Boc-protected intermediate II-11. Reaction with a chlorinating agent such as 1,3-dichloro-5,5-dimethylhydantoin (II-12) or N-chlorosuccinimide in a mixture of solvents such as acetonitrile, water, and acetic acid followed by reaction with amine II-13 in the presence of a base such as triethylamine or diisopropylamine in a solvent such as acetonitrile afforded intermediate II-14. Removal of the Boc protecting group with an acid such as HCl in dioxane and DCM or TFA in DCM gave amine II-16. Reaction of acid I-9 with a chlorinating agent such as thionyl chloride or oxalyl chloride gave acid chloride 11-15. Reaction of 11-15 with 11-16 in the presence of a base such as triethylamine or diisopropylethylamine in a solvent such as THE or DCM afforded compounds of Formula Io with L=SO₂NH.

### EXAMPLES

### Synthesis of ethyl 2-bromopyrazolo[3,2-b][1,3]thiazole-7-carboxylate (intermediate A)

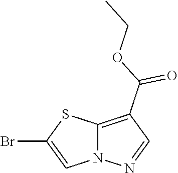

### Step a: 5-bromo-2-methyl-1,3-thiazole

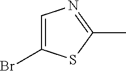

Into a 2-L 4-necked round-bottom flask, was placed 2-methylthiazole (150.00 g, 1361.56 mmol), DMF (1.17 L), NBS (290.8 g, 1633.89 mmol). The resulting solution was stirred for 8 h at room temperature. The reaction was then quenched by the addition of 1500 mL of water. The resulting solution was extracted with 3×500 mL of Et₂O and the organic layer was concentrated. The product was precipitated by the addition of n-heptane (300 mL). The solids were collected by filtration. This resulted in 99 g (40.8%) of 5-bromo-2-methyl-1,3-thiazole as a brown solid. LC-MS: (ES, m/z): [M+H]⁺=178

### Step b: ethyl 2-(5-bromo-1,3-thiazol-2-yl)acetate

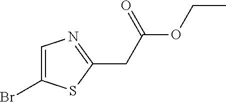

Into a 2-L 4-necked round-bottom flask, was placed 5-bromo-2-methyl-1,3-thiazole (99.00 g, 556.02 mmol), THE (1100 mL). This was followed by the addition of LiHMDS (667.23 mL, 667.23 mmol) dropwise with stirring at −60° C. in 30 min. Diethyl carbonate (75.64 g, 667.23 mmol) was added dropwise to the mixture with stirring at −60° C. in 30 min. The resulting solution was stirred for 1 h at room temperature. The reaction was then quenched by the addition of 1100 mL of water. The resulting solution was extracted with 3×500 mL of ethyl acetate and the organic layer was concentrated. The residue was purified by a silica gel column with ethyl acetate/petroleum ether (1:50). The fractions were combined and concentrated to give 40.1 g (28.8%) of ethyl 2-(5-bromo-1,3-thiazol-2-yl)acetate as a yellow solid.

LC-MS: (ES, m/z): [M+H]⁺=250

### Step c: 3-amino-5-bromo-2-(2-ethoxy-2-oxoethyl)-1,3-thiazol-3-ium 2,4,6-trimethylbenzenesulfonate

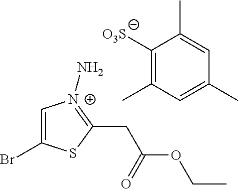

(Z)-(ethyl N-[(2,4,6-trimethylbenzenesulfonyl)oxy]ethanimidate) (51.34 g, 179.92 mmol) was added at 0° C. was added to a mixture of TFA (235.15 g, 2398.9 mmol) and ice water (50 mL) at 0° C. and stirred for 1.5 h. Ice water (300 mL) was then added. The solid was collected by filtration. The solid was dissolved into DCM and dried with anhydrous Na₂SO₄. Then the organic phase was collected through filtration, a solution of ethyl 2-(5-bromo-1,3-thiazol-2-yl)acetate (40.1 g, 160.32 mmol) in DCM (300 mL) was added dropwise. Then the resulting solution was stirred at r.t for 1.5 h. The white precipitate was collected by filtration, washed with MTBE (1×50 mL), dried to give 39.5 g (91.3%) of 3-amino-5-bromo-2-(2-ethoxy-2-oxoethyl)-1,3-thiazol-3-ium 2,4,6-trimethylbenzenesulfonate as a white solid. LC-MS: (ES, m/z): [M+H]⁺=265

### Step d: ethyl 2-bromopyrazolo[3,2-b][1,3]thiazole-7-carboxylate (Intermediate A)

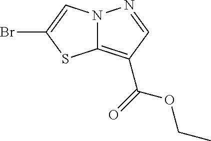

3-amino-5-bromo-2-(2-ethoxy-2-oxoethyl)-1,3-thiazol-3-ium 2,4,6-trimethylbenzenesulfonate (39.50 g, 84.87 mmol), triethyl orthoformate (150 mL) was placed into a 500-mL round-bottom flask. The resulting solution was stirred for 2 h at 120° C. and concentrated. The residue was purified by a silica gel column with ethyl acetate/petroleum ether (1:50). The fractions were combined, concentrated, dried to give 9.0 g (38.6%) of ethyl 2-bromopyrazolo[3,2-b][1,3]thiazole-7-carboxylate as a light pink solid. LC-MS: (ES, m/z): 275 [M+H]⁺; ¹H-NMR: (300 MHz, CDCl₃, ppm): δ 8.21 (s, 1H), 7.86 (s, 1H), 4.36 (q, J=7.1 Hz, 2H), 1.40 (t, J=7.1 Hz, 3H).

### Example 1. N-(5-((2-(2-azabicyclo[2.2.2]octan-2-yl)ethyl)carb-amoyl)-2-methylpyridin-3-yl)-2-(1-methyl-1H-pyrazol-4-yl)pyrazolo[5,1-b]thiazole-7-carboxamide

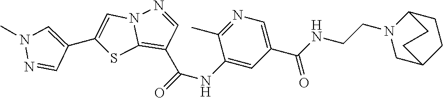 [SMILES: Cc1ncc(C(=O)NCCN2CC3CCC2CC3)cc1NC(=O)c1cnn2cc(-c3cnn(C)c3)sc12]

### Step a: 2-(2-Azabicyclo[2.2.2]octan-2-yl)acetonitrile

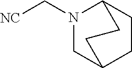 [SMILES: N#CCN1CC2CCC1CC2]

To a solution of 2-azabicyclo[2.2.2]octane (500 mg, 4.50 mmol) and potassium carbonate (1.4 g, 9.9 mmol) in N,N-dimethylformamide (10 mL) was added 2-bromoacetonitrile (593 mg, 4.9 mmol) at room temperature. The resulting mixture was stirred at 55° C. for 16 h before cooling to room-temperature. The resulting mixture was quenched with water (5 mL) and extracted with ethyl acetate (10 mL×3). The combined organic phases were dried over anhydrous Na₂SO₄ and filtered. The filtration was concentrated under reduced pressure to give crude product, which was purified by column chromatography over silica gel (eluent: petroleum ether:ethyl acetate=3:1) to give the title compound 2-(2-azabicyclo[2.2.2]octan-2-yl)acetonitrile (500 mg, 74%) as a colorless oil. ¹H NMR (400 MHz, CDCl₃) δ 3.48 (s, 2H), 2.80-2.86 (m, 2H), 2.59-2.66 (m, 1H), 1.95-2.03 (m, 2H), 1.60-1.74 (m, 3H), 1.43-1.59 (m, 4H).

### Step b: 2-(2-Azabicyclo[2.2.2]octan-2-yl)ethan-1-amine

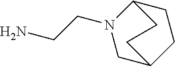 [SMILES: NCCN1CC2CCC1CC2]

To a solution of 2-(2-azabicyclo[2.2.2]octan-2-yl)acetonitrile (450 mg, 3.0 mmol) in THF(10 mL) was added lithium aluminium hydride (170 mg, 4.5 mmol) by portions at 0° C. (ice/water), and the resulting mixture was stirred for 90 min at 20° C. After cooled to 0° C., the reaction mixture was quenched with water (250 mg) and filtered. The filtration was then concentrated to dryness under reduced pressure to afford the crude product 2-(2-azabicyclo[2.2.2]octan-2-yl)ethan-1-amine (350 mg, 75%) as a colorless oil. ¹H NMR (400 MHz, CDCl₃) δ 2.57-2.73 (m, 4H), 2.47-2.55 (m, 2H), 1.79-1.94 (m, 2H), 1.34-1.63 (m, 10H).

### Step c: 2-Bromopyrazolo[5,1-b]thiazole-7-carboxylic acid

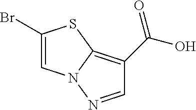 [SMILES: O=C(O)c1cnn2cc(Br)sc12]

To a solution of ethyl 2-bromopyrazolo[5,1-b]thiazole-7-carboxylate (3.1 g, 11.3 mmol) in ethanol (6 mL) was added sodium hydroxide (11.3 ml, 2M in water, 22.6 mmol) at room temperature. The reaction mixture was stirred at 40° C. for 16 h before cooling to room-temperature. The mixture was adjusted to pH=3-4 with HCl (aq, 2 M). The mixture was filtered and washed with water (10 mL×3). The solid was evaporated under vacuum to give the desired product 2-bromopyrazolo[5,1-b]thiazole-7-carboxylic acid (2.8 g, 97%) as white solid. LCMS (ESI): mass calcd. for C₆H₃BrN₂O₂S, 245.9; m/z found, 247 [M+H]+.

### Step d: Ethyl 5-(2-bromopyrazolo[5,1-b]thiazole-7-carboxamido)-6-methylnicotinate

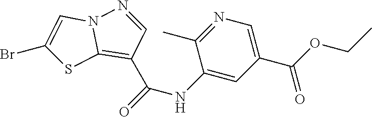 [SMILES: CCOC(=O)c1cnc(C)c(NC(=O)c2cnn3cc(Br)sc23)c1]

A mixture of 2-bromopyrazolo[5,1-b]thiazole-7-carboxylic acid (1 g, 4.04 mmol) in thionyl chloride (28 ml, 393 mmol) was stirred at 70° C. After stirred for 1 h at 70° C., the reaction mixture was concentrated under vacuum to give the crude product 2-bromopyrazolo[5,1-b]thiazole-7-carbonyl chloride as white solid. To a solution of ethyl 5-amino-6-methylnicotinate (680 mg, 3.8 mmol), TEA (2.1 ml, 15.0 mmol) in THE (10 mL) was added 2-bromopyrazolo[5,1-b]thiazole-7-carbonyl chloride (1 g, 3.8 mmol) at room-temperature. The resulting mixture was stirred at room-temperature for 1 h before quenched with cooled H₂O. The mixture was extracted with ethyl acetate (30 ml*3). The organic extracts were dried over anhydrous Na₂SO₄ and filtered. The filtration was concentrated under reduced pressure to give crude product, which was purified by column chromatography over silica gel (eluent: petroleum ether:ethyl acetate=3:1) to give the title compound ethyl 5-(2-bromopyrazolo[5,1-b]thiazole-7-carboxamido)-6-methylnicotinate (800 mg, 52%) as a yellow solid. LCMS (ESI): mass calcd. for C₁₅H₁₃BrN₄O₃S, 409; m/z found, 411 [M+H]+. ¹H NMR (400 MHz, CDCl₃) δ 8.93 (d, J=1.76 Hz, 1H), 8.79 (d, J=1.76 Hz, 1H), 8.08 (s, 1H), 7.88 (s, 1H), 7.33 (s, 1H), 4.34-4.43 (m, 2H), 2.64 (s, 3H), 1.39 (t, J=7.17 Hz, 3H).

### Step e: 6-methyl-5-(2-(1-methyl-1H-pyrazol-4-yl)pyrazolo[5,1-b]thiazole-7-carboxamido)-nicotinic acid

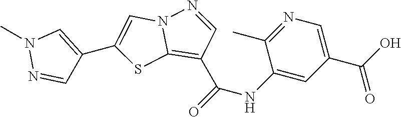 [SMILES: Cc1ncc(C(=O)O)cc1NC(=O)c1cnn2cc(-c3cnn(C)c3)sc12]

To a solution of ethyl 5-(2-bromopyrazolo[5,1-b]thiazole-7-carboxamido)-6-methylnicotinate (200 mg, 0.49 mmol) and 1-methyl-4-(4,4,5,5-tetramethyl-1,3,2-dioxaborolan-2-yl)pyrazole (120 mg, 0.58 mmol) in 1,4-dioxane (20 mL) and H₂O (5 mL) was added [1,1-bis(diphenylphosphino)ferrocene]palladium(II) chloride dichloromethane complex (36 mg, 0.049 mmol) and K₃PO₄ (310 mg, 1.46 mmol) under N₂. The resulting mixture was stirred at 90° C. under N₂ for 3 h before cooled to 25° C. The reaction mixture was diluted with ethyl acetate (30 mL) and filtered over celite. The filtration was concentrated to the crude product, which was treated with ethyl acetate (90 mL) and water (30 mL). The organic phase was washed with 3 M HCl aqueous solution, dried over anhydrous Na₂SO₄ and filtered. The filtration was concentrated under reduced pressure to give the crude product, which was purified by preparative high-performance liquid chromatography over Column: Xtimate C18 100*30 mm*3 um to give the title compound 6-methyl-5-(2-(1-methyl-1H-pyrazol-4-yl)pyrazolo[5,1-b]thiazole-7-carboxamido)-nicotinic acid (100 mg, 52%) as a white solid. LCMS (ESI): mass calcd. for C₁₇H₁₄N₆O₃S, 382; m/z found, 383.1 [M+H]+.

### Step f: N-(5-((2-(2-azabicyclo[2.2.2]octan-2-yl)ethyl)carb-amoyl)-2-methylpyridin-3-yl)-2-(1-methyl-1H-pyrazol-4-yl)pyrazolo[5,1-b]thiazole-7-carboxamide

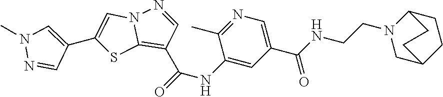 [SMILES: Cc1ncc(C(=O)NCCN2CC3CCC2CC3)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (80 mg, 0.209 mmol), HATU (80 mg, 0.21 mmol) and N,N-diisopropylethylamine (56 mg, 0.44 mmol) in DMF (5 mL) was added 2-(2-azabicyclo[2.2.2]octan-2-yl)ethan-1-amine (27 mg, 0.17 mmol). The resulting mixture was stirred at 30° C. for 1 h and then concentrated under vacuum to give the crude product, which was purified by preparative high-performance liquid chromatography over Column: Phenomenex Gemini-NX 80*40 mm*3 um to give the title compound N-(5-((2-(2-azabicyclo[2.2.2]octan-2-yl)ethyl)carb-amoyl)-2-methylpyridin-3-yl)-2-(1-methyl-1H-pyrazol-4-yl)pyrazolo[5,1-b]thiazole-7-carboxamide (65 mg, 71%) as a white solid. LCMS (ESI): mass calcd. for C₂₆H₃₀N₈O₂S, 518.2; m/z found, 519.2 [M+H]⁺. ¹H NMR (400 MHz, DMSO-d6) δ 10.00 (s, 1H), 8.77 (br s, 2H), 8.56 (s, 1H), 8.52 (s, 1H), 8.19 (s, 1H), 8.16 (s, 1H), 7.85 (s, 1H), 3.84 (s, 3H), 3.46 (s, 3H), 2.86 (m, 2H), 2.49 (m, 5H), 1.93 (m, 2H), 1.72 (m, 1H), 1.51 (m, 6H).

### Example 2. N-(5-((2-(4-azaspiro[2.4]heptan-4-yl)ethyl)carbamoyl)-2-methylpyridin-3-yl)-2-(1-methyl-1H-pyrazol-4-yl)pyrazolo[5,1-b]thiazole-7-carboxamide

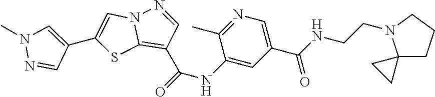 [SMILES: Cc1ncc(C(=O)NCCN2CCCC23CC3)cc1NC(=O)c1cnn2cc(-c3cnn(C)c3)sc12]

### Step a: 2-(4-azaspiro[2.4]heptan-4-yl)acetonitrile

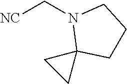

To a solution of 4-azaspiro[2.4]heptane-hydrochloride (400 mg, 3.0 mmol) and potassium carbonate (827 mg, 6.0 mmol) in N,N-dimethylformamide (6 mL) was added 2-bromoacetonitrile (430 mg, 3.6 mmol) at room-temperature. The resulting mixture was stirred at 60° C. for 16 h before cooling to room temperature. The resulting mixture was quenched with water (5 ml) and extracted with ethyl acetate (10 ml*3). The organic extracts were dried over anhydrous Na₂SO₄ and filtered. The filtration was concentrated under reduced pressure to give crude product, which was purified by column chromatography over silica gel (eluent: petroleum ether:ethyl acetate=4:1) to give the title compound 2-(4-azaspiro[2.4]heptan-4-yl)acetonitrile (350 mg, 85%) as a colorless oil.

### Step b: 2-(4-azaspiro[2.4]heptan-4-yl)ethan-1-amine

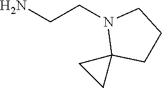

To a solution of 2-(4-azaspiro[2.4]heptan-4-yl)acetonitrile (350 mg, 2.6 mmol) in THE (8 mL) was added lithium aluminium hydride (107 mg, 2.8 mmol) by portions at 0° C. (ice/water). The resultant mixture was stirred at 20° C. for 90 min before quenched with water (100 mg) at 0° C. The reaction mixture was filtered, the filtration was concentrated to dryness under reduced pressure to afford the crude product 2-(4-azaspiro[2.4]heptan-4-yl)ethan-1-amine as a colorless oil, which was used to the next step without further purification. LCMS (ESI): mass calcd. for C₈H₁₆N₂, 140.3; m/z found, 141.1 [M+H]+.

### Step c: N-(5-((2-(4-azaspiro[2.4]heptan-4-yl)ethyl)carbamoyl)-2-methylpyridin-3-yl)-2-(1-methyl-1H-pyrazol-4-yl)pyrazolo[5,1-b]thiazole-7-carboxamide

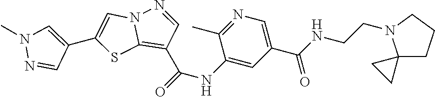 [SMILES: Cc1ncc(C(=O)NCCN2CCCC23CC3)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (100 mg, 0.26 mmol), HATU (119 mg, 0.31 mmol) and N,N-diisopropylethylamine (101 mg, 0.78 mmol) in N,N-dimethylformamide (4 mL) was added 2-(4-azaspiro[2.4]heptan-4-yl)ethan-1-amine (48 mg, 0.28 mmol). The mixture stirred at 25° C. for 2 h and then concentrated under vacuum to give the crude product, which was purified by preparative high-performance liquid chromatography over Column: Boston Green ODS 150*30 mm*5 um to give the title compound N-(5-((2-(4-azaspiro[2.4]heptan-4-yl)ethyl)carbamoyl)-2-methylpyridin-3-yl)-2-(1-methyl-1H-pyrazol-4-yl)pyrazolo[5,1-b]thiazole-7-carboxamide (45 mg, 31%) as a white solid. LCMS (ESI): mass calcd. for C₂₅H₂₈N₈O₂S, 504.2; m/z found, 505.1 [M+H]⁺. ¹H NMR (400 MHz, METHANOL-d4) δ 8.78 (d, J=2.0 Hz, 1H), 8.42 (s, 1H), 8.33 (d, J=2.0 Hz, 1H), 8.25 (s, 1H), 8.04 (s, 1H), 7.84-7.81 (m, 1H), 3.95 (s, 3H), 3.69-3.63 (m, 2H), 3.48-3.40 (m, 2H), 2.96 (br t, J=6.5 Hz, 2H), 2.63-2.61 (m, 3H), 2.20-2.11 (m, 2H), 2.09-2.02 (m, 2H), 1.17-1.11 (m, 2H), 0.82-0.76 (m, 2H).

### Example 3. N-(5-((2-(5-azaspiro[3.4]octan-5-yl)ethyl)carbamoyl)-2-methylpyridin-3-yl)-2-(1-methyl-1H-pyrazol-4-yl)pyrazolo[5,1-b]thiazole-7-carboxamide

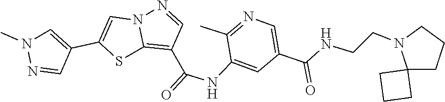 [SMILES: Cc1ncc(C(=O)NCCN2CCCC23CCC3)cc1NC(=O)c1cnn2cc(-c3cnn(C)c3)sc12]

### Step a: tert-butyl (2-(5-azaspiro[3.4]octan-5-yl)ethyl)carbamate

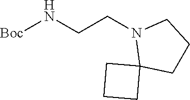 [SMILES: CC(C)(C)OC(=O)NCCN1CCCC12CCC2]

To a solution of 5-azaspiro[3.4]octane hemioxalate (250 mg, 0.8 mmol) and potassium carbonate (550 mg, 4.00 mmol) in acetonitrile (3 mL) was added tert-butyl (2-bromoethyl)carbamate (360 mg, 1.60 mmol) at room-temperature. The resulting mixture was stirred at 80° C. for 12 h before cooling to room temperature. The reaction was concentrated under reduced pressure to give crude product, which was purified by column chromatography over silica gel (eluent: ethyl acetate:methanol=10:1) to give the title compound tert-butyl (2-(5-azaspiro[3.4]octan-5-yl)ethyl)carbamate (200 mg, 98%) as a pale yellow solid. LCMS (ESI): mass calcd. for C₁₄H₂₆N₂O₂, 254.3; m/z found, 255.3 [M+H]+.

### Step b: 2-(5-azaspiro[3.4]octan-5-yl)ethanamine

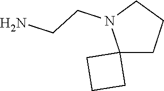 [SMILES: NCCN1CCCC12CCC2]

To a solution of tert-butyl (2-(5-azaspiro[3.4]octan-5-yl)ethyl)carbamate (200 mg, 0.826 mmol) in THE (5 mL) was added HCl/dioxane (5 mL, 4M) at 0° C. The resultant mixture was stirred at 20° C. for 15 hours at 25° C. The reaction mixture was concentrated under reduced pressure to afford the crude product 2-(5-azaspiro[3.4]octan-5-yl)ethanamine as a HCl salt white solid. ¹H NMR (400 MHz, DMSO-d6) δ 11.30 (br s, 1H), 8.56 (br s, 3H), 4.33-4.25 (m, 1H), 3.62-3.49 (m, 2H), 3.46 (br d, J=8.3 Hz, 1H), 3.20 (br s, 2H), 2.70-2.58 (m, 1H), 2.49-2.40 (m, 1H), 2.21-2.11 (m, 2H), 2.04-1.89 (m, 4H), 1.82 (dt, J=5.0, 9.9 Hz, 2H).

### Step c: N-(5-((2-(5-azaspiro[3.4]octan-5-yl)ethyl)carbamoyl)-2-methylpyridin-3-yl)-2-(1-methyl-1H-pyrazol-4-yl)pyrazolo[5,1-b]thiazole-7-carboxamide

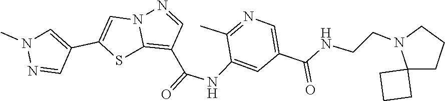 [SMILES: Cc1ncc(C(=O)NCCN2CCCC23CCC3)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (70 mg, 0.18 mmol), 2-(5-azaspiro[3.4]octan-5-yl)ethanamine (31 mg, 0.20 mmol) and N,N-diisopropylethylamine (101 mg, 0.78 mmol) in N,N-dimethylformamide (3 mL) was added HATU (84 mg, 0.22 mmol). The mixture stirred at 25° C. for 2 h and then concentrated under vacuum to give the crude product, which was purified by preparative high-performance liquid chromatography over Column: Welch Xtimate C18 150*25 mm*5 um to give the title compound N-(5-((2-(5-azaspiro[3.4]octan-5-yl)ethyl)carbamoyl)-2-methylpyridin-3-yl)-2-(1-methyl-1H-pyrazol-4-yl)pyrazolo[5,1-b]thiazole-7-carboxamide (28.6 mg, 28%) as a white solid. LCMS (ESI): mass calcd. for C₂₆H₃₀N₈O₂S, 518.6; m/z found, 519.1 [M+H]⁺. ¹H NMR (400 MHz, METHANOL-d4) δ 9.02 (d, J=2.0 Hz, 1H), 8.92 (d, J=2.0 Hz, 1H), 8.47 (s, 1H), 8.27 (s, 1H), 8.04 (s, 1H), 7.83 (s, 1H), 3.95 (s, 3H), 3.91-3.85 (m, 1H), 3.80-3.71 (m, 2H), 3.65-3.56 (m, 1H), 3.23-3.14 (m, 1H), 2.81 (s, 3H), 2.65-2.52 (m, 2H), 2.42-2.33 (m, 1H), 2.24-1.93 (m, 8H).

### Example 4. N-(5-((2-(9-azabicyclo[3.3.1]nonan-9-yl)ethyl)carbamoyl)-2-methylpyridin-3-yl)-2-(1-methyl-1H-pyrazol-4-yl)pyrazolo[5,1-b]thiazole-7-carboxamide

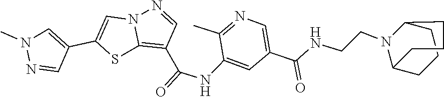 [SMILES: Cc1ncc(C(=O)NCCN2C3CCCC2CCC3)cc1NC(=O)c1cnn2cc(-c3cnn(C)c3)sc12]

### Step a: 2-(9-azabicyclo[3.3.1]nonan-9-yl)acetonitrile

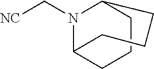 [SMILES: N#CCN1C2CCCC1CCC2]

To a solution of 9-azabicyclo[3.3.1]nonane hydrochloride (200 mg, 1.24 mmol) and potassium carbonate (340 mg, 2.5 mmol) in N,N-dimethylformamide (3 mL) was added 2-bromoacetonitrile (220 mg, 1.8 mmol) at room-temperature. The resulting mixture was stirred at 60° C. for 16 h before cooling to room temperature. The resulting mixture was quenched with water (5 ml) and extracted with ethyl acetate (10 ml*3). The organic extracts were dried over anhydrous Na₂SO₄ and filtered. The filtration was concentrated under reduced pressure to give crude product, which was purified by column chromatography over silica gel (eluent: petroleum ether:ethyl acetate=4:1) to give the title compound 2-(9-azabicyclo[3.3.1]nonan-9-yl)acetonitrile (180 mg, 88%) as a colorless oil. ¹H NMR (400 MHz, CDCl₃) δ 3.63 (s, 2H), 2.93 (br s, 2H), 1.87-1.99 (m, 6H), 1.50-1.71 (m, 6H).

### Step b: 2-(9-azabicyclo[3.3.1]nonan-9-yl)ethan-1-amine

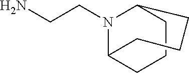 [SMILES: NCCN1C2CCCC1CCC2]

To a solution of 2-(9-azabicyclo[3.3.1]nonan-9-yl)acetonitrile (180 mg, 1.1 mmol) in THE (10 mL) was added lithium aluminium hydride (60 mg, 1.6 mmol) by portions at 0° C. (ice/water). The resultant mixture was stirred at 20° C. for 90 min before quenched with water (100 mg) at 0° C. The reaction mixture was filtered, the filtration was concentrated to dryness under reduced pressure to afford the crude product 2-(9-azabicyclo[3.3.1]nonan-9-yl)ethan-1-amine as a colorless oil, which was used to the next step without further purification.

### Step c: N-(5-((2-(9-azabicyclo[3.3.1]nonan-9-yl)ethyl)carbamoyl)-2-methylpyridin-3-yl)-2-(1-methyl-1H-pyrazol-4-yl)pyrazolo[5,1-b]thiazole-7-carboxamide

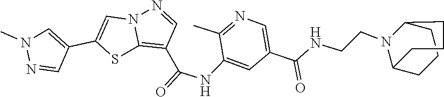 [SMILES: Cc1ncc(C(=O)NCCN2C3CCCC2CCC3)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (100 mg, 0.26 mmol), HATU (120 mg, 0.31 mmol) and N,N-diisopropylethylamine (100 mg, 0.78 mmol) in N,N-dimethylformamide (4 mL) was added 2-(9-azabicyclo[3.3.1]nonan-9-yl)ethan-1-amine (42 mg, 0.25 mmol). The mixture stirred at 25° C. for 2 h and then concentrated under vacuum to give the crude product, which was purified by preparative high-performance liquid chromatography over Column: Boston Green ODS 150*30 mm*5 um to give the title compound N-(5-((2-(9-azabicyclo[3.3.1]nonan-9-yl)ethyl)carbamoyl)-2-methylpyridin-3-yl)-2-(1-methyl-1H-pyrazol-4-yl)pyrazolo[5,1-b]thiazole-7-carboxamide (30 mg, 26%) as a white solid. LCMS (ESI): mass calcd. for C₂₇H₃₂N₈O₂S, 532; m/z found, 533.2 [M+H]+. ¹H NMR (400 MHz, DMSO-d6) δ 9.95 (s, 1H), 8.74 (d, J=1.98 Hz, 1H), 8.55 (s, 1H), 8.50 (s, 1H), 8.14-8.19 (m, 2H), 7.85 (s, 1H), 3.84 (s, 3H), 2.73-3.01 (m, 4H), 2.48 (m, 6H), 1.91 (m, 6H), 1.38-1.57 (m, 6H).

### Example 5. N-(5-((2-(3-azabicyclo[3.1.1]heptan-3-yl)ethyl)carbamoyl)-2-methylpyridin-3-yl)-2-(1-methyl-1H-pyrazol-4-yl)pyrazolo[5,1-b]thiazole-7-carboxamide

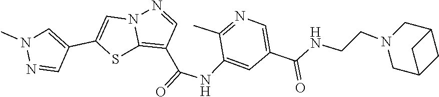 [SMILES: Cc1ncc(C(=O)NCCN2CC3CC(C3)C2)cc1NC(=O)c1cnn2cc(-c3cnn(C)c3)sc12]

### Step a: 2-(3-azabicyclo[3.1.1]heptan-3-yl)acetonitrile

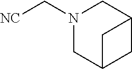

To a solution of 3-azabicyclo[3.1.1]heptane hydrochloride (250 mg, 1.87 mmol) and potassium carbonate (510 mg, 3.74 mmol) in N,N-dimethylformamide (3 mL) was added 2-bromoacetonitrile (330 mg, 2.8 mmol) at room-temperature. The resulting mixture was stirred at 60° C. for 16 h before cooling to room temperature. The resulting mixture was quenched with water (5 ml) and extracted with ethyl acetate (10 ml*3). The organic extracts were dried over anhydrous Na₂SO₄ and filtered. The filtration was concentrated under reduced pressure to give crude product, which was purified by column chromatography over silica gel (eluent: petroleum ether:ethyl acetate=4:1) to give the title compound 2-(3-azabicyclo[3.1.1]heptan-3-yl)acetonitrile (220 mg, 86%) as a colorless oil. ¹H NMR (400 MHz, CDCl₃) δ 3.52-3.60 (m, 2H), 2.96 (s, 4H), 2.29-2.39 (m, 2H), 1.96-2.08 (m, 2H), 1.43-1.53 (m, 2H).

### Step b: 2-(3-azabicyclo[3.1.1]heptan-3-yl)ethan-1-amine

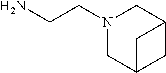

To a solution of 2-(3-azabicyclo[3.1.1]heptan-3-yl)acetonitrile (350 mg, 2.6 mmol) in THE (8 mL) was added lithium aluminium hydride (107 mg, 2.8 mmol) by portions at 0° C. (ice/water). The resultant mixture was stirred at 20° C. for 90 min before quenched with water (100 mg) at 0° C. The reaction mixture was filtered. And the filtration was concentrated to dryness under reduced pressure to afford the crude product 2-(3-azabicyclo[3.1.1]heptan-3-yl)ethan-1-amine as a colorless oil, which was used to the next step without further purification.

### Step c: 5-(2-bromopyrazolo[5,1-b]thiazole-7-carboxamido)-6-methylnicotinic acid

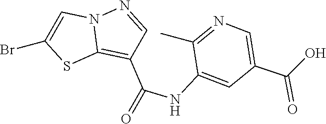 [SMILES: Cc1ncc(C(=O)O)cc1NC(=O)c1cnn2cc(Br)sc12]

To a solution of ethyl 5-(2-bromopyrazolo[5,1-b]thiazole-7-carboxamido)-6-methylnicotinate (600 mg, 1.47 mmol) in ethanol (10 mL) was added NaOH (1 N, 3 mL), the reaction was stirred at 50° C. for 2 h. Solvent was evaporated under reduced pressure, water was added (5 mL), adjusted to pH=3˜4 with HCl (aq, 2 M). The mixture was filtered, washed with H₂O, dried under reduced pressure to give the crude product 5-(2-bromopyrazolo[5,1-b]thiazole-7-carboxamido)-6-methylnicotinic acid (300 mg, 50%). LCMS (ESI): mass calcd. for C₁₃H₉BrN₄O₃S, 381.2; m/z found, 381 [M+H]⁺.

### Step d: N-(5-((2-(3-azabicyclo[3.1.1]heptan-3-yl)ethyl)carbamoyl)-2-methylpyridin-3-yl)-2-bromopyrazolo[5,1-b]thiazole-7-carboxamide

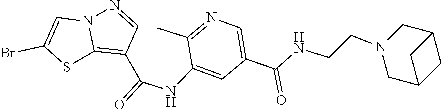 [SMILES: Cc1ncc(C(=O)NCCN2CC3CC(C3)C2)cc1NC(=O)c1cnn2cc(Br)sc12]

To a solution of 5-(2-bromopyrazolo[5,1-b]thiazole-7-carboxamido)-6-methylnicotinic acid (160 mg, 0.42 mmol), HATU(190 mg, 0.5 mmol) and N,N-diisopropylethylamine (140 mg, 1.05 mmol) in N,N-dimethylformamide (5 mL) was added 2-(3-azabicyclo[3.1.1]heptan-3-yl)ethan-1-amine (70 mg, 0.5 mmol). The mixture stirred at 25° C. for 2 h and then concentrated under vacuum to give the crude product, which was purified by preparative high-performance liquid chromatography over Column: Phenomenex Gemini-NX 80*40 mm*3 um to give the title compound N-(5-((2-(3-azabicyclo[3.1.1]heptan-3-yl)ethyl)carbamoyl)-2-methylpyridin-3-yl)-2-bromopyrazolo[5,1-b]thiazole-7-carboxamide (150 mg, 70%) as a white solid. LCMS (ESI): mass calcd. for C₂₁H₂₃BrN₆O₂S, 503.4; m/z found, 503 [M+H]⁺.

### Step e: N-(5-((2-(3-azabicyclo[3.1.1]heptan-3-yl)ethyl)carbamoyl)-2-methylpyridin-3-yl)-2-(1-methyl-1H-pyrazol-4-yl)pyrazolo[5,1-b]thiazole-7-carboxamide

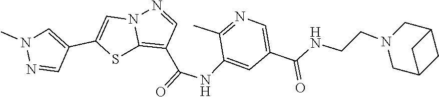 [SMILES: Cc1ncc(C(=O)NCCN2CC3CC(C3)C2)cc1NC(=O)c1cnn2cc(-c3cnn(C)c3)sc12]

To a solution of N-(5-((2-(3-azabicyclo[3.1.1]heptan-3-yl)ethyl)carbamoyl)-2-methylpyridin-3-yl)-2-bromopyrazolo[5,1-b]thiazole-7-carboxamide (100 mg, 0.2 mmol) and 1-methyl-4-(4,4,5,5-tetramethyl-1,3,2-dioxaborolan-2-yl)pyrazole (50 mg, 0.24 mmol) in 1,4-dioxane (12 mL) and H₂O (3 mL) was added [1,1-bis(diphenylphosphino)ferrocene]palladium(II) chloride dichloromethane complex (30 mg, 0.04 mmol) and K₃PO₄ (130 mg, 0.6 mmol) under N₂. The resulting mixture was stirred at 90° C. under N₂ for 3 h before cooled to 25° C. The reaction mixture was diluted with ethyl acetate (30 mL) and filtered over celite. The filtration was concentrated to the crude product, which was treated with ethyl acetate (90 mL) and water (30 mL). The organic phase was washed with 3 M HCl aqueous solution, dried over anhydrous Na₂SO₄ and filtered. The filtration was concentrated under reduced pressure to give the crude product, which was purified by preparative high-performance liquid chromatography over Column: Welch Xtimate C18 150*30 mm*5 um to give the title compound N-(5-((2-(3-azabicyclo[3.1.1]heptan-3-yl)ethyl)carbamoyl)-2-methylpyridin-3-yl)-2-(1-methyl-1H-pyrazol-4-yl)pyrazolo[5,1-b]thiazole-7-carboxamide (35 mg, 34%) as a white solid. LCMS (ESI): mass calcd. for C₂₅H₂₈N₈O₂S, 504; m/z found, 505.1 [M+H]⁺. ¹H NMR (400 MHz, METHANOL-d4) δ 8.76 (d, J=1.98 Hz, 1H), 8.39 (s, 1H), 8.30 (d, J=1.98 Hz, 1H), 8.22 (s, 1H), 8.01 (s, 1H), 7.80 (s, 1H), 3.92 (s, 3H), 3.62 (br t, J=6.50 Hz, 2H), 3.16 (br s, 4H), 2.97 (br s, 2H), 2.59 (s, 3H), 2.40 (br s, 2H), 2.13 (br s, 2H), 1.58 (br d, J=6.62 Hz, 1H), 1.50-1.67 (m, 1H).

### Example 6. 2-(1-cyclopropyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide

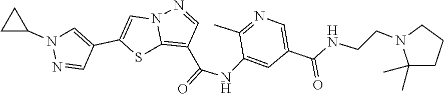 [SMILES: Cc1ncc(C(=O)NCCN2CCCC2(C)C)cc1NC(=O)c1cnn2cc(-c3cnn(C4CC4)c3)sc12]

### Step a: 5-amino-6-methylnicotinic acid

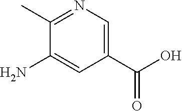

To a solution of ethyl 5-amino-6-methylnicotinate (5.00 g, 27.75 mmol) in ethanol (15 mL) was added sodium hydroxide (27 ml, 2M in water, 55.5 mmol) at room temperature. The reaction mixture was stirred at 50° C. for 15 min before cooling to room-temperature. The mixture was adjusted to pH=3-4 with HCl (aq, 2 M). The mixture was filtered and washed with water (10 mL×3). The solid was evaporated under vacuum to give the desired product 5-amino-6-methylnicotinic acid (4.2 g, 99%) as yellow solid. ¹H NMR (400 MHz, DMSO-d6) δ 8.14 (d, J=1.54 Hz, 1H), 7.48 (d, J=1.76 Hz, 1H), 2.48 (br s, 2H), 2.33 (s, 3H).

### Step b: 2-(2,2-dimethylpyrrolidin-1-yl)acetonitrile

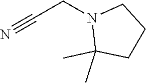

To a solution of 2,2-dimethylpyrrolidine (20 g, 201 mmol) and potassium carbonate (55.74 g, 403 mmol) in DMF (130 mL) was added 2-bromoacetonitrile (15.4 mL, 221 mmol) at room-temperature. The resulting mixture was stirred at 30° C. for 12 h. The reaction was concentrated under reduced pressure to give crude product, which was purified by column chromatography over silica gel (eluent: ethyl acetate:methanol=7:3) to give the title compound 2-(2,2-dimethylpyrrolidin-1-yl)acetonitrile (22 g, 79%) as a pale yellow oil. ¹H NMR (400 MHz, DMSO-d6) δ 3.50 (s, 2H), 2.81-2.86 (m, 2H), 1.69-1.77 (m, 2H), 1.56-1.62 (m, 2H), 0.98 (s, 6H).

### Step c: 2-(2,2-dimethylpyrrolidin-1-yl)ethanamine

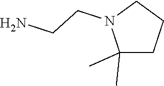

To a solution of 2-(2,2-dimethylpyrrolidin-1-yl)acetonitrile (22 g, 159.18 mmol) in THE (400 mL) was added lithium aluminium hydride (7.25 g, 191.01 mmol) by portions at 0° C. (ice/water). The resultant mixture was stirred at 20° C. for 4 hours before quenched with water (7.25 g) at 0° C. The reaction mixture was filtered. And the filtration was concentrated to dryness under reduced pressure to afford the crude product 2-(2,2-dimethylpyrrolidin-1-yl)ethanamine a yellow oil. ¹H NMR (400 MHz, DMSO-d6) δ 2.71-2.81 (m, 2H), 2.63-2.71 (m, 2H), 2.44-2.50 (m, 2H), 1.71-1.82 (m, 2H), 1.59-1.69 (m, 2H), 0.96-1.02 (m, 6H).

### Step d: 5-amino-N-(2-(2,2-dimethylpyrrolidin-1-yl)ethyl)-6-methylnicotinamide

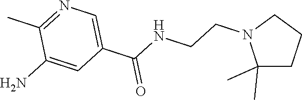 [SMILES: Cc1ncc(C(=O)NCCN2CCCC2(C)C)cc1N]

To a solution of 5-amino-6-methylnicotinic acid (5.4 g, 35.5 mmol), 2-(2,2-dimethylpyrrolidin-1-yl)ethanamine (5.05 g, 35.5 mmol) and N,N-diisopropylethylamine (18.3 g, 142 mmol) in DMF (50 mL) was added HATU (27.0 g, 71 mmol). The resulting mixture was stirred at 30° C. for 16 hours and then concentrated under vacuum to give the crude product, which was purified by preparative high-performance liquid chromatography over Column: SANPONT C18, 250*80 mm*10 um, 100 A to give the title compound 5-amino-N-(2-(2,2-dimethylpyrrolidin-1-yl)ethyl)-6-methylnicotinamide (10 g, 68%) as a yellow solid. LCMS (ESI): mass calcd. for C₁₅H₂₄N₄O, 276.3; m/z found, 277.3 [M+H]⁺.

### Step e: 2-Bromopyrazolo[5,1-b]thiazole-7-carboxylic acid

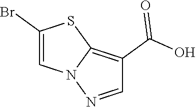 [SMILES: O=C(O)c1cnn2cc(Br)sc12]

To a solution of ethyl 2-bromopyrazolo[5,1-b]thiazole-7-carboxylate (3.1 g, 11.3 mmol) in ethanol (6 mL) was added sodium hydroxide (11.3 ml, 2M in water, 22.6 mmol) at room temperature. The reaction mixture was stirred at 40° C. for 16 h before cooling to room-temperature. The mixture was adjusted to pH=3-4 with HCl (aq, 2 M). The mixture was filtered and washed with water (10 mL×3). The solid was evaporated under vacuum to give desired product 2-bromopyrazolo[5,1-b]thiazole-7-carboxylic acid (2.8 g, 97%) as white solid. LCMS (ESI): mass calcd. for C₆H₃BrN₂O₂S, 245.9; m/z found, 247 [M+H]⁺.

### Step f: 2-bromo-N-(5-((2-(2,2-dimethylpyrrolidin-1-yl)ethyl)carbamoyl)-2-methylpyridin-3-yl)pyrazolo[5,1-b]thiazole-7-carboxamide

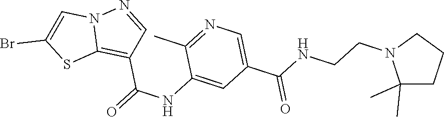 [SMILES: Cc1ncc(C(=O)NCCN2CCCC2(C)C)cc1NC(=O)c1cnn2cc(Br)sc12]

A mixture of 2-bromopyrazolo[5,1-b]thiazole-7-carboxylic acid (1 g, 4.1 mmol) in thionyl chloride (28 ml, 393 mmol) was stirred at 70° C. for 2 h. The reaction mixture was concentrated under vacuum to give the crude product as yellow solid 2-bromopyrazolo[5,1-b]thiazole-7-carbonyl chloride. 2-bromopyrazolo[5,1-b]thiazole-7-carbonyl chloride (1 g, 3.6 mmol) was added to a solution consisting of 5-amino-N-(2-(2,2-dimethylpyrrolidin-1-yl)ethyl)-6-methylnicotinamide (1.49 g, 3.6 mmol), TEA(1.51 ml, 10.8 mmol) and THE (10 mL) at room-temperature, The resulting mixture was stirred at 60° C. for 12 h. The resulting mixture was quenched with cooled H₂O and extracted with ethyl acetate (30 ml*3). The organic extracts were dried over anhydrous Na₂SO₄, and filtered, the filtration was concentrated under reduced pressure to give crude product, which was purified by column chromatography over silica gel (eluent: dichloromethane/methyl alcohol=4:1) to give the title compound 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (1 g, 55%) as a yellow solid. LCMS (ESI): mass calcd. for C₂₁H₂₅BrN₆O₂S, 505.4; m/z found, 507.2 [M+H]+.

### Step g: 2-(1-cyclopropyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide

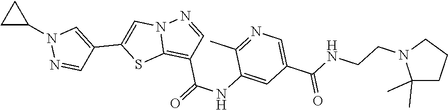 [SMILES: Cc1ncc(C(=O)NCCN2CCCC2(C)C)cc1NC(=O)c1cnn2cc(-c3cnn(C4CC4)c3)sc12]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (50 mg, 0.099 mmol) and 1-cyclopropyl-4-(4,4,5,5-tetramethyl-1,3,2-dioxaborolan-2-yl)-1H-pyrazole (28 mg, 0.12 mmol) in 1,4-dioxane (1 mL) and H₂O (0.25 mL) was added [1,1-bis(diphenylphosphino)ferrocene]palladium(II) chloride dichloromethane complex (8 mg, 0.01 mmol) and NaHCO₃ (25 mg, 0.30 mmol) under N₂. The resulting mixture was stirred at 90° C. for 3 h before cooled to 25° C. The reaction mixture was diluted with ethyl acetate (10 mL) and filtered over celite. The filtration was concentrated to the crude product, which was treated with dichloromethane (10 mL) and water (10 mL). The organic phase was washed with 3 M HCl aqueous solution, dried over anhydrous Na₂SO₄ and filtered. The filtration was concentrated under reduced pressure to give the crude product, which was purified by preparative high-performance liquid chromatography over Column: Boston Prime C18 150*30 mm*5 um to give the title compound 2-(1-cyclopropyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide (16 mg, 30%) as a yellow oil. LCMS (ESI): mass calcd. for C₂₇H₃₂N₈O₂S, 532.6; m/z found, 533.3 [M+H]⁺. ¹H NMR (400 MHz, METHANOL-d4) δ 8.75 (d, J=1.8 Hz, 1H), 8.39 (s, 1H), 8.29 (d, J=1.8 Hz, 1H), 8.22 (s, 1H), 8.11 (s, 1H), 7.80 (s, 1H), 3.79-3.64 (m, 1H), 3.53 (br t, J=6.4 Hz, 2H), 2.97 (br s, 2H), 2.74 (br s, 2H), 2.59 (s, 3H), 1.85 (br s, 2H), 1.73 (br d, J=7.7 Hz, 2H), 1.12 (br d, J=2.6 Hz, 2H), 1.11-1.03 (m, 8H).

### Example 7. N-(5-((2-(2,2-dimethylpyrrolidin-1-yl)ethyl)carbamoyl)-2-methyl-pyridin-3-yl)-2-(pyridin-4-yl)pyrazolo[5,1-b]thiazole-7-carboxamide

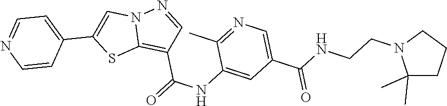 [SMILES: Cc1ncc(C(=O)NCCN2CCCC2(C)C)cc1NC(=O)c1cnn2cc(-c3ccncc3)sc12]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (100 mg, 0.20 mmol) and 4-(4,4,5,5-tetramethyl-1,3,2-dioxaborolan-2-yl)pyridine (49 mg, 0.24 mmol) in 1,4-dioxane (3 mL) and H₂O (0.75 mL) was added [1,1-bis(diphenylphosphino)ferrocene]palladium(II) chloride dichloromethane complex (16 mg, 0.2 mmol) and K₃PO₄ (126 mg, 0.59 mmol) under N₂. The resulting mixture was stirred at 90° C. for 3 h before cooled to 25° C. The reaction mixture was diluted with ethyl acetate (10 mL) and filtered over celite. The filtration was concentrated to the crude product, which was treated with dichloromethane (10 mL) and water (10 mL). The organic phase was washed with 3 M HCl aqueous solution, dried over anhydrous Na₂SO₄ and filtered. The filtration was concentrated under reduced pressure to give the crude product, which was purified by preparative high-performance liquid chromatography over Column: Boston Prime C18 150*30 mm*5 um to give the title compound N-(5-((2-(2,2-dimethylpyrrolidin-1-yl)ethyl)carbamoyl)-2-methylpyridin-3-yl)-2-(pyridin-4-yl)pyrazolo[5,1-b]thiazole-7-carboxamide (12 mg, 12%) as a white solid. LCMS (ESI): mass calcd. for C₂₆H₂₉N₇O₂S, 503.6; m/z found, 504.3 [M+H]⁺. ¹H NMR (400 MHz, METHANOL-d4) δ 8.92 (s, 1H), 8.80 (d, J=1.8 Hz, 1H), 8.65 (d, J=6.0 Hz, 2H), 8.53 (s, 1H), 8.34 (d, J=2.0 Hz, 1H), 7.78 (d, J=6.0 Hz, 2H), 3.55 (t, J=7.0 Hz, 2H), 2.95 (br s, 2H), 2.77-2.69 (m, 2H), 2.64 (s, 3H), 1.92-1.83 (m, 2H), 1.77-1.70 (m, 2H), 1.08 (s, 6H).

### Example 8. N-(5-((2-(2,2-dimethylpyrrolidin-1-yl)ethyl)carbamoyl)-2-methylpyridin-3-yl)-2-(1-methyl-1H-pyrazol-4-yl)pyrazolo[5,1-b]thiazole-7-carboxamide

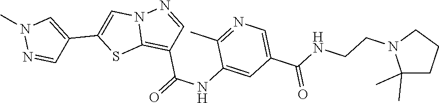 [SMILES: Cc1ncc(C(=O)NCCN2CCCC2(C)C)cc1NC(=O)c1cnn2cc(-c3cnn(C)c3)sc12]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (250 mg, 0.50 mmol) and 1-methyl-4-(4,4,5,5-tetramethyl-1,3,2-dioxaborolan-2-yl)-1H-pyrazole (49 mg, 0.24 mmol) in 1,4-dioxane (4 mL) and H₂O (1 mL) was added [1,1-bis(diphenylphosphino)ferrocene]palladium(II) chloride dichloromethane complex (40 mg, 0.05 mmol) and K₂CO₃ (205 mg, 1.48 mmol) under N₂. The resulting mixture was stirred at 100° C. for 12 h before cooled to 25° C. The reaction mixture was diluted with ethyl acetate (10 mL) and filtered over celite. The filtration was concentrated to the crude product, which was treated with dichloromethane (10 mL) and water (10 mL). The organic phase was washed with 3 M HCl aqueous solution, dried over anhydrous Na₂SO₄ and filtered. The filtration was concentrated under reduced pressure to give the crude product, which was purified by preparative high-performance liquid chromatography over Column: Boston Prime C18 150*30 mm*5 um to give the title compound N-(5-((2-(2,2-dimethylpyrrolidin-1-yl)ethyl)carbamoyl)-2-methylpyridin-3-yl)-2-(1-methyl-1H-pyrazol-4-yl)pyrazolo[5,1-b]thiazole-7-carboxamide (80 mg, 32%) as a red solid. LCMS (ESI): mass calcd. for C₂₅H₃₀N₈O₂S, 506.6; m/z found, 507.4 [M+H]⁺. ¹H NMR (400 MHz, METHANOL-d4) δ 8.79 (d, J=2.0 Hz, 1H), 8.43 (s, 1H), 8.32 (d, J=2.0 Hz, 1H), 8.27 (s, 1H), 8.06 (s, 1H), 7.85 (s, 1H), 3.97 (s, 3H), 3.54 (t, J=7.0 Hz, 2H), 2.92 (br t, J=7.3 Hz, 2H), 2.70 (br t, J=6.7 Hz, 2H), 2.63 (s, 3H), 1.90-1.81 (m, 2H), 1.75-1.68 (m, 2H), 1.07 (s, 6H).

### Example 9. N-(5-((2-(2,2-dimethylpyrrolidin-1-yl)ethyl)carbamoyl)-2-methylpyridin-3-yl)-2-(1-methyl-1H-pyrazol-3-yl)pyrazolo[5,1-b]thiazole-7-carboxamide

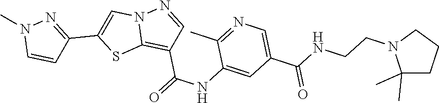 [SMILES: Cc1ncc(C(=O)NCCN2CCCC2(C)C)cc1NC(=O)c1cnn2cc(-c3ccn(C)n3)sc12]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (150 mg, 0.30 mmol) and 1-methyl-3-(4,4,5,5-tetramethyl-1,3,2-dioxaborolan-2-yl)-1H-pyrazole (74 mg, 0.36 mmol) in 1,4-dioxane (3 mL) and H₂O (0.75 mL) was added [1,1-bis(diphenylphosphino)ferrocene]palladium(II) chloride dichloromethane complex (24 mg, 0.03 mmol) and K₃PO₄ (189 mg, 0.89 mmol) under N₂. The resulting mixture was stirred at 95° C. for 12 h before cooled to 25° C. The reaction mixture was diluted with ethyl acetate (10 mL) and filtered over celite. The filtration was concentrated to the crude product, which was treated with dichloromethane (10 mL) and water (10 mL). The organic phase was washed with 3 M HCl aqueous solution, dried over anhydrous Na₂SO₄ and filtered. The filtration was concentrated under reduced pressure to give the crude product, which was purified by preparative high-performance liquid chromatography over Column: Boston Prime C18 150*30 mm*5 um to give the title compound N-(5-((2-(2,2-dimethylpyrrolidin-1-yl)ethyl)carbamoyl)-2-methylpyridin-3-yl)-2-(1-methyl-1H-pyrazol-3-yl)pyrazolo[5,1-b]thiazole-7-carboxamide (26 mg, 17%) as a white solid. LCMS (ESI): mass calcd. for C₂₅H₃₀N₈O₂S, 506.6; m/z found, 507.2 [M+H]⁺. ¹H NMR (400 MHz, METHANOL-d4) δ 8.79 (d, J=2.0 Hz, 1H), 8.44 (d, J=7.3 Hz, 2H), 8.34 (d, J=2.0 Hz, 1H), 7.69 (d, J=2.0 Hz, 1H), 6.71 (d, J=2.3 Hz, 1H), 3.95 (s, 3H), 3.56 (br t, J=7.0 Hz, 2H), 3.02-2.90 (m, 2H), 2.73 (br s, 2H), 2.63 (s, 3H), 1.93-1.82 (m, 2H), 1.76-1.66 (m, 2H), 1.09 (s, 6H).

### Example 10. N-(5-(3-(2,2-dimethylpyrrolidin-1-yl)propanamido)-2-methyl-pyridin-3-yl)-2-(1-methyl-1H-pyrazol-4-yl)pyrazolo[5,1-b]thiazole-7-carboxamide

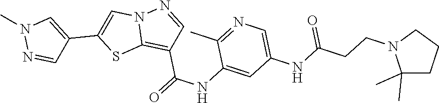 [SMILES: Cc1ncc(NC(=O)CCN2CCCC2(C)C)cc1NC(=O)c1cnn2cc(-c3cnn(C)c3)sc12]

### Step a: N-(6-methyl-5-nitropyridin-3-yl)acrylamide

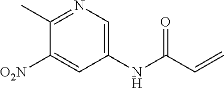

To a solution of 6-methyl-5-nitropyridin-3-amine hydrochloride (2 g, 10.5 mmol) and 2-chloroacetyl chloride (1 ml, 10.5 mmol) in dichloromethane (30 ml) was cooled to 0° C. and then trimethylamine (2.9 ml, 21.1 mmol) was added dropwise. After the reaction was warmed to 25° C. with stirred for 12 hours. The mixture was filtered through a celite pad and the filtrate was evaporated under vacuum to give residue. The crude product was purified by flash column chromatography over silica gel (eluent: petroleum ether/ethyl acetate from 100/0 to 50/50). The desired fractions were collected, and the solvent was concentrated to dryness in vacuum to give title compound (600 mg, 27%) as yellow solid. LCMS (ESI): mass calcd. for C₉H₉N₃O₃, 207.18; m/z found, 207.9 [M+H]⁺. ¹H NMR (400 MHz, CDCl₃-d) δ 11.67 (br s, 1H), 8.97 (br s, 1H), 8.90-8.73 (m, 1H), 6.49-6.38 (m, 2H), 5.77 (br d, J=7.5 Hz, 1H), 2.74 (s, 3H).

### Step b: 3-(2,2-dimethylpyrrolidin-1-yl)-N-(6-methyl-5-nitropyridin-3-yl)propanamide

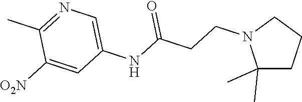 [SMILES: Cc1ncc(NC(=O)CCN2CCCC2(C)C)cc1[N+](=O)[O-]]

To a solution of N-(6-methyl-5-nitropyridin-3-yl)acrylamide (820 mg, 3.96 mmol), 2,2-dimethylpyrrolidine hydrochloride (540 mg, 3.96 mmol), potassium carbonate (3.28 g, 23.75 mmol) in acetonitrile (5 mL) was added potassium iodide (66 mg, 0.40 mmol). The reaction was stirred at 80° C. for 12 hours. The mixture was filtered through a celite pad and the filtrate was evaporated under vacuum to give residue. The crude product was purified by flash column chromatography over silica gel (eluent: ethyl acetate/methanol from 100/0 to 90/10) to give title compound 3-(2,2-dimethylpyrrolidin-1-yl)-N-(6-methyl-5-nitropyridin-3-yl)propanamide (900 mg, 74%) as yellow solid. LCMS (ESI): mass calcd. for C₁₅H₂₂N₄O₃, 306.36; m/z found, 307.1 [M+H]+. ¹H NMR (400 MHz, CDCl₃-d) δ 11.88 (br s, 1H), 8.77 (d, J=2.3 Hz, 1H), 8.55 (d, J=2.0 Hz, 1H), 2.89 (br s, 2H), 2.81 (br d, J=5.2 Hz, 2H), 2.71 (s, 3H), 2.60 (br s, 2H), 1.91-1.82 (m, 2H), 1.82-1.75 (m, 2H), 1.08 (s, 6H).

### Step c: N-(5-amino-6-methylpyridin-3-yl)-3-(2,2-dimethylpyrrolidin-1-yl)propanamide

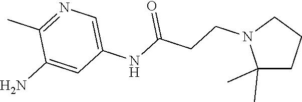 [SMILES: Cc1ncc(NC(=O)CCN2CCCC2(C)C)cc1N]

To a solution of 3-(2,2-dimethylpyrrolidin-1-yl)-N-(6-methyl-5-nitropyridin-3-yl)propanamide (900 mg, 2.9 mmol) in methanol (10 mL) was hydrogenated at 25° C. (15 psi) with Pd/C (125 mg, 10%) as a catalyst in the presence of H₂ for 24 hours. After uptake of H₂ (3 eq), the catalyst filtered off and the filtrate was evaporated give the title compound (644 mg, 76%) as yellow oil. LCMS (ESI): mass calcd. for C₁₅H₂₄N₄O, 276.2; m/z found, 277.2 [M+H]⁺. ¹H NMR (400 MHz, METHANOL-d4) δ 7.85 (d, J=2.2 Hz, 1H), 7.41 (d, J=2.2 Hz, 1H), 2.89 (br s, 2H), 2.83 (br s, 2H), 2.54 (br t, J=6.9 Hz, 2H), 2.28 (s, 3H), 1.90-1.78 (m, 2H), 1.77-1.67 (m, 2H), 1.08 (s, 6H).

### Step d: 2-bromo-N-(5-(3-(2,2-dimethylpyrrolidin-1-yl)propanamido)-2-methylpyridin-3-yl) pyrazolo[5,1-b]thiazole-7-carboxamide

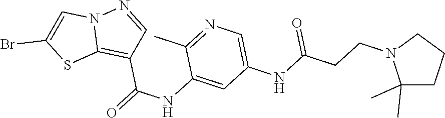 [SMILES: Cc1ncc(NC(=O)CCN2CCCC2(C)C)cc1NC(=O)c1cnn2cc(Br)sc12]

To a solution of 2-bromopyrazolo[5,1-b]thiazole-7-carboxylic acid (486 mg, 1.9 mmol) in thionyl chloride (4 ml, 55 mmol) was stirred at 70° C. for 1 hour. The reaction mixture was concentrated under vacuum to give the crude product as white solid 2-bromopyrazolo[5,1-b]thiazole-7-carbonyl chloride. 2-bromopyrazolo[5,1-b]thiazole-7-carbonyl chloride (486 mg, 1.8 mmol) was added to a solution consisting of N-(5-amino-6-methylpyridin-3-yl)-3-(2,2-dimethylpyrrolidin-1-yl)propanamide (600 mg, 2.1 mmol), TEA (795 uL, 5.7 mmol) and THE (8 mL) at 60° C. for 2 hours. The resulting mixture was quenched with cooled H₂O and extracted with ethyl acetate (30 ml*3). The organic extracts were dried over anhydrous Na₂SO₄, and filtered, the filtrate was concentrated under reduced pressure to give crude product, which was purified by preparative high-performance liquid chromatography over Column: Xtimate C18 100*30 mm*3 um to give the title compound (650 mg, 62%) as a yellow solid. LCMS (ESI): mass calcd. for C₂₁H₂₅BrN₆O₂S, 505.4; m/z found, 507.0 [M+H]⁺. ¹H NMR (400 MHz, METHANOL-d4) δ 8.55 (d, J=2.4 Hz, 1H), 8.44 (s, 1H), 8.32 (s, 1H), 8.24 (d, J=2.2 Hz, 1H), 3.46 (br s, 4H), 2.90 (br t, J=6.3 Hz, 2H), 2.47 (s, 3H), 2.14-1.98 (m, 4H), 1.43 (br s, 6H).

### Step e: N-(5-(3-(2,2-dimethylpyrrolidin-1-yl)propanamido)-2-methylpyridin-3-yl)-2-(1-methyl-1H-pyrazol-4-yl)pyrazolo[5,1-b]thiazole-7-carboxamide

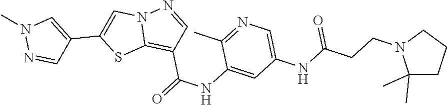 [SMILES: Cc1ncc(NC(=O)CCN2CCCC2(C)C)cc1NC(=O)c1cnn2cc(-c3cnn(C)c3)sc12]

To a solution of 2-bromo-N-(5-(3-(2,2-dimethylpyrrolidin-1-yl)propanamido)-2-methylpyridin-3-yl) pyrazolo[5,1-b]thiazole-7-carboxamide (50 mg, 0.10 mmol) and 1-methyl-4-(4,4,5,5-tetramethyl-1,3,2-dioxaborolan-2-yl)pyrazole (25 mg, 0.12 mmol) in 1,4-dioxane (5 mL) was added [1,1-bis(diphenylphosphino)ferrocene]palladium(II) chloride dichloromethane complex (8.1 mg, 0.01 mmol) and sodium bicarbonate solution (199 uL, 0.40 mmol, 2 M) under N₂, the yellow mixture stirred at 100° C. for 12 hours. The mixture was cooled to 25° C., and then diluted with ethyl acetate (30 mL), filtered and the filtrate was concentrated to remove solvent to give a residue. The crude product was purified by preparative high-performance liquid chromatography over Column: Phenomenex Gemini NX-C18 75*30 mm*3 um to give the title compound (30 mg, 58%) as a white solid. LCMS (ESI): mass calcd. for C₂₅H₃₀N₈O₂S, 506.6; m/z found, 507.2 [M+H]⁺. ¹H NMR (400 MHz, METHANOL-d4) δ 8.51 (d, J=2.0 Hz, 1H), 8.36 (s, 1H), 8.26-8.15 (m, 2H), 8.00 (s, 1H), 7.80 (s, 1H), 3.92 (s, 3H), 2.94-2.76 (m, 4H), 2.58 (br t, J=6.7 Hz, 2H), 2.47 (s, 3H), 1.89-1.77 (m, 2H), 1.77-1.64 (m, 2H), 1.08 (s, 6H).

### Example 11. N-(5-(3-(2,2-dimethylpyrrolidin-1-yl)propanamido)-2-methyl-pyridin-3-yl)-2-(1-methyl-1H-pyrazol-5-yl)pyrazolo[5,1-b]thiazole-7-carboxamide

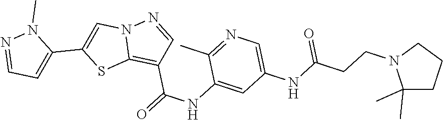 [SMILES: Cc1ncc(NC(=O)CCN2CCCC2(C)C)cc1NC(=O)c1cnn2cc(-c3ccnn3C)sc12]

To a solution of 2-bromo-N-(5-(3-(2,2-dimethylpyrrolidin-1-yl)propanamido)-2-methylpyridin-3-yl)pyrazolo[5,1-b]thiazole-7-carboxamide (100 mg, 0.20 mmol) and 1-methyl-5-(4,4,5,5-tetramethyl-1,3,2-dioxaborolan-2-yl)pyrazole (62 mg, 0.30 mmol) in 1,4-dioxane (10 mL) was added [1,1-bis(diphenylphosphino)ferrocene]palladium(II) chloride dichloromethane complex (32 mg, 0.04 mmol) and sodium bicarbonate solution (396 uL, 0.79 mmol, 2 M) under N₂, the yellow mixture stirred at 100° C. for 12 hours. The mixture was cooled to 25° C., and then diluted with ethyl acetate (30 mL), filtered and the filtrate was concentrated to remove solvent to give a residue. The crude product was purified by preparative high-performance liquid chromatography over Column: Phenomenex Gemini NX-C18 80*30 mm*3 um to give the title compound (41 mg, 40%) as a white solid. LCMS (ESI): mass calcd. for C₂₅H₃₀N₈O₂S, 506.6; m/z found, 507.1 [M+H]⁺. ¹H NMR (400 MHz, METHANOL-d4) δ 8.56 (d, J=2.1 Hz, 1H), 8.52-8.45 (m, 2H), 8.26 (d, J=1.9 Hz, 1H), 7.58 (d, J=2.0 Hz, 1H), 6.65 (d, J=2.0 Hz, 1H), 4.06 (s, 3H), 2.92 (br d, J=18.1 Hz, 4H), 2.63 (br t, J=6.7 Hz, 2H), 2.51 (s, 3H), 1.94-1.82 (m, 2H), 1.81-1.72 (m, 2H), 1.13 (s, 6H).

### Example 12. N-(5-(3-(2,2-dimethylpyrrolidin-1-yl)propanamido)-2-methyl-pyridin-3-yl)-2-(1-methyl-1H-pyrazol-3-yl)pyrazolo[5,1-b]thiazole-7-carboxamide

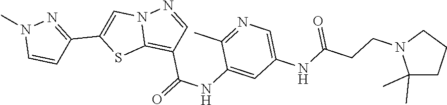 [SMILES: Cc1ncc(NC(=O)CCN2CCCC2(C)C)cc1NC(=O)c1cnn2cc(-c3ccn(C)n3)sc12]

To a solution of 2-bromo-N-(5-(3-(2,2-dimethylpyrrolidin-1-yl)propanamido)-2-methylpyridin-3-yl)pyrazolo[5,1-b]thiazole-7-carboxamide (50 mg, 0.10 mmol) and 1-methyl-3-(4,4,5,5-tetramethyl-1,3,2-dioxaborolan-2-yl)-1H-pyrazole (31 mg, 0.15 mmol) in 1,4-dioxane (5 mL) was added [1,1-bis(diphenylphosphino)ferrocene]palladium(II) chloride dichloromethane complex (16 mg, 0.02 mmol) and Sodium Bicarbonate solution (198 uL, 0.40 mmol, 2 M) under N₂, the yellow mixture stirred at 100° C. for 12 hours. The mixture was cooled to 25° C., and then diluted with ethyl acetate (30 mL), filtered and the filtrate was concentrated to remove solvent to give a residue. The crude product was purified by preparative high-performance liquid chromatography over Column: Phenomenex Gemini NX-C18 80*30 mm*3 um to give the title compound (19 mg, 38%) as a white solid. LCMS (ESI): mass calcd. for C₂₅H₃₀N₈O₂S, 506.6; m/z found, 507.1 [M+H]⁺. ¹H NMR (400 MHz, METHANOL-d4) δ 8.53 (br d, J=2.2 Hz, 1H), 8.38 (d, J=2.2 Hz, 2H), 8.20 (d, J=2.2 Hz, 1H), 7.64 (d, J=2.2 Hz, 1H), 6.67 (d, J=2.2 Hz, 1H), 3.91 (s, 3H), 2.85 (br s, 4H), 2.60 (br d, J=6.4 Hz, 2H), 2.48 (s, 3H), 1.83 (br d, J=7.5 Hz, 2H), 1.74 (br d, J=7.9 Hz, 2H), 1.09 (s, 6H).

### Example 13. N-(5-(2-(2,2-dimethylpyrrolidin-1-yl)acetamido)-2-methylpyridin-3-yl)-2-(1-methyl-1H-pyrazol-4-yl)pyrazolo[5,1-b]thiazole-7-carboxamide

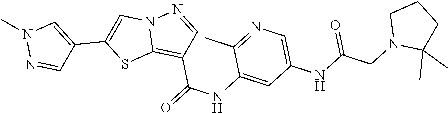

### Step a: tert-butyl (6-methyl-5-nitropyridin-3-yl)carbamate

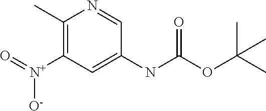

To a solution of 5-bromo-2-methyl-3-nitropyridine (22.0 g, 101 mmol), tert-butyl carbamate (14.2 g, 122 mmol) and cesium carbonate (46.2 g, 142 mmol) in dioxane (500 mL) was added dicyclohexyl (2′,4′,6′-triisopropyl-[1,1′-biphenyl]-2-yl)phosphine (21.7 g, 45.6 mmol) and tris(dibenzylideneacetone) dipalladium(0) (13.9 g, 15.2 mmol) under nitrogen at room-temperature. The resulting mixture was stirred at 100° C. for 16 h. the mixture was cooled to room temperature and evaporated in vacuum to afford crude product as black solid. The residue was purified by silica gel column chromatography (eluent: petroleum ether/ethyl acetate=100:0 to 70:30). The desired fractions were collected, and the solvent was concentrated to dryness under vacuum to afford the crude product. Petroleum ether (500 mL) was added to the crude product. The mixture was stirred at room temperature for 30 min. The mixture was filtered, the filtered cake was washed with petroleum ether (200 mL*2). The filter cake was dried in vacuum to afford desired product tert-butyl (6-methyl-5-nitropyridin-3-yl)carbamate (19.9 g, 100%) as white solid. LCMS (ESI): mass calcd. for C₁₁H₁₅N₃O₄, 253.2; m/z found, 254.0 [M+H]+.

### Step b: tert-butyl (5-amino-6-methylpyridin-3-yl)carbamate

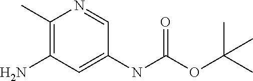

To a solution of tert-butyl (6-methyl-5-nitropyridin-3-yl)carbamate (5.0 g, 19.7 mmol) in methanol (50 mL) was added palladium 10% on activated carbon (1.66 g, 1.56 mmol) under nitrogen at room-temperature. The resulting mixture was hydrogenated at 25° C. (atmospheric pressure) for 16 h. The reaction mixture was filtered and the filtrate was evaporated under vacuum to afford desired product tert-butyl (5-amino-6-methylpyridin-3-yl)carbamate (4.8 g, 92%) as white solid. LCMS (ESI): mass calcd. for C₁₁H₁₇N₃O₂, 223.2; m/z found, 224.1 [M+H]⁺.

### Step c: tert-butyl (5-(2-bromopyrazolo[5,1-b]thiazole-7-carboxamido)-6-methylpyridin-3-yl)carbamate

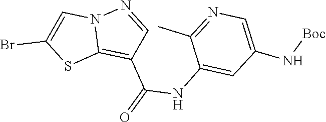 [SMILES: Cc1ncc(NC(=O)OC(C)(C)C)cc1NC(=O)c1cnn2cc(Br)sc12]

To a solution of 2-bromopyrazolo[5,1-b]thiazole-7-carboxylic acid (4.5 g, 18.08 mmol) in thionyl chloride (40 mL), The resulting mixture was stirred at 70° C. for 1 h before cooling to room-temperature. The reaction mixture was concentrated under vacuum to give the crude product as white solid 2-bromopyrazolo[5,1-b]thiazole-7-carbonyl chloride. To a solution of tert-butyl (5-amino-6-methylpyridin-3-yl)carbamate (3.6 g, 16.1 mmol) and TEA (6.73 ml, 48.3 mmol) in THE (720 mL) was added 2-bromopyrazolo[5,1-b]thiazole-7-carbonyl chloride at room-temperature, The resulting mixture was stirred at 95° C. for 16 h before cooling to room-temperature. The resulting mixture was evaporated in vacuum to afford crude product. The residue was purified by silica gel column chromatography (eluent: petroleum ether/ethyl acetate/methanol=100:0:0 to 0:90:10). The desired fractions were collected and the solvent was concentrated to dryness under vacuum to afford desired product tert-butyl (5-(2-bromopyrazolo[5,1-b]thiazole-7-carboxamido)-6-methylpyridin-3-yl)carbamate (6.4 g, 85%) as a yellow solid. LCMS (ESI): mass calcd. for C₁₇H₁₈BrN₅O₃S, 452.3; m/z found, 453. [M+H]+.

### Step d: tert-butyl (6-methyl-5-(2-(1-methyl-1H-pyrazol-4-yl)pyrazolo[5,1-b]thiazole-7-carboxamido)pyridin-3-yl)carbamate

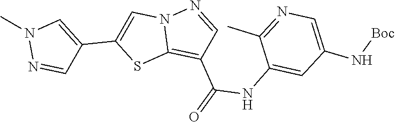 [SMILES: Cc1ncc(NC(=O)OC(C)(C)C)cc1NC(=O)c1cnn2cc(-c3cnn(C)c3)sc12]

To a solution of tert-butyl (5-(2-bromopyrazolo[5,1-b]thiazole-7-carboxamido)-6-methylpyridin-3-yl)carbamate (5.8 g, 11.8 mmol), 1-methyl-4-(4,4,5,5-tetramethyl-1,3,2-dioxaborolan-2-yl)-1H-pyrazole (2.94 g, 14.1 mmol) and Cs₂CO₃ (11.5 g, 35.4 mmol) in 1,4-dioxane (192 mL) and H₂O (48 mL) was added [1,1-bis(diphenylphosphino)ferrocene]palladium(II) chloride dichloromethane complex (1.44 g, 1.77 mmol) under nitrogen, the mixture stirred at 100° C. for 16 h. The mixture was cooled to 25° C. and concentrated under vacuum to give crude product. The crude product was purified by silica gel column chromatography (eluent: petroleum ether/ethyl acetate/methanol=100:0:0 to 0:90:10). The desired fractions were collected and the solvent was concentrated to dryness under vacuum to afford the title compound tert-butyl (6-methyl-5-(2-(1-methyl-1H-pyrazol-4-yl) pyrazolo[5,1-b]thiazole-7-carboxamido)pyridin-3-yl)carbamate (2.72 g, 97%) as a yellow solid. LCMS (ESI): mass calcd. for C₂₁H₂₃N₇O₃S, 453.5; m/z found, 454.0 [M+H]⁺.

### Step e: N-(5-amino-2-methylpyridin-3-yl)-2-(1-methyl-1H-pyrazol-4-yl)pyrazolo[5,1-b]thiazole-7-carboxamide

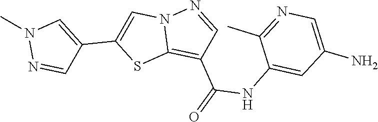 [SMILES: Cc1ncc(N)cc1NC(=O)c1cnn2cc(-c3cnn(C)c3)sc12]

A mixture of tert-butyl (6-methyl-5-(2-(1-methyl-1H-pyrazol-4-yl)pyrazolo[5,1-b]thiazole-7-carboxamido) pyridin-3-yl)carbamate (2.72 g, 5.80 mmol) in HCl/dioxane (22.8 mL, 4 M, 91.20 mmol) was stirred at 25° C. for 16 h. The mixture was concentrated in vacuum to give the title compound N-(5-amino-2-methylpyridin-3-yl)-2-(1-methyl-1H-pyrazol-4-yl)pyrazolo[5,1-b]thiazole-7-carboxamide (2.2 g, 91%) as a yellow solid. LCMS (ESI): mass calcd. for C₁₆H₁₅N₇OS, 353.4; m/z found, 354.0 [M+H]⁺.

### Step f: N-(5-(2-chloroacetamido)-2-methylpyridin-3-yl)-2-(1-methyl-1H-pyrazol-4-yl)pyrazolo[5,1-b]thiazole-7-carboxamide

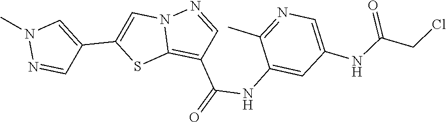 [SMILES: Cc1ncc(NC(=O)CCl)cc1NC(=O)c1cnn2cc(-c3cnn(C)c3)sc12]

To a solution of N-(5-amino-2-methylpyridin-3-yl)-2-(1-methyl-1H-pyrazol-4-yl)pyrazolo[5,1-b]thiazole-7-carboxamide (2.2 g, 4.7 mmol) and NaHCO₃ (1.57 g, 3.0 mmol) in DMF (20 mL) was added 2-chloroacetyl chloride (0.596 mL, 7.02 mL) at 0° C. The mixture was stirred at 25° C. for 16 h. The mixture was filtered through Celite, rinsed with DMF (3 mL). The filtrate was concentrated to afford crude product. Ethyl acetate (10 mL) and sat. NaHCO₃ (20 mL) was added slowly. The mixture was stirred at room-temperature for 10 min. The mixture was filtered, rinsed with water (10 mL). The filter cake was dried in vacuum to give the title compound N-(5-(2-chloroacetamido)-2-methylpyridin-3-yl)-2-(1-methyl-1H-pyrazol-4-yl)pyrazolo[5,1-b]thiazole-7-carboxamide (1.85 g, 97%) as a gray solid. LCMS (ESI): mass calcd. for C₁₈H₁₆ClN₇O₂S, 429.8; m/z found, 430.0.

### Step g: N-(5-(2-(2,2-dimethylpyrrolidin-1-yl)acetamido)-2-methylpyridin-3-yl)-2-(1-methyl-1H-pyrazol-4-yl)pyrazolo[5,1-b]thiazole-7-carboxamide

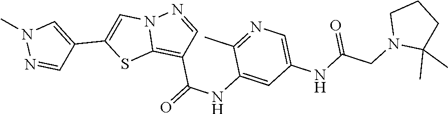

To a solution of N-(5-(2-chloroacetamido)-2-methylpyridin-3-yl)-2-(1-methyl-1H-pyrazol-4-yl)pyrazolo[5,1-b]thiazole-7-carboxamide (100 mg, 0.23 mmol) and 2,2-dimethylpyrrolidine (37.86 mg, 0.28 mmol) in DMF (1.5 mL) was added K₂CO₃ (96 mg, 0.70) and NaI (21 mg, 0.14 mmol) at room-temperature. The mixture was stirred at 50° C. for 1.5 h. The mixture was filtered through Celite, rinsed with DMF (3 mL). The filtrate was concentrated to afford crude product. The crude product was purified by preparative high-performance liquid chromatography over Column: Phenomenex Gemini-NX 80*40 mm*3 um to give the title compound N-(5-(2-(2,2-dimethylpyrrolidin-1-yl)acetamido)-2-methylpyridin-3-yl)-2-(1-methyl-1H-pyrazol-4-yl)pyrazolo[5,1-b]thiazole-7-carboxamide (63 mg, 98%) as a gray solid. LCMS (ESI): mass calcd. for C₂₄H₂₈N₈O₂S, 492.6; m/z found, 493.3[M+H]⁺. ¹H NMR (400 MHz, DMSO-d6) δ 9.75 (br s, 2H), 8.63 (d, J=2.27 Hz, 1H), 8.57 (s, 1H), 8.51 (s, 1H), 8.19 (s, 1H), 8.16 (d, J=2.15 Hz, 1H), 7.88 (s, 1H), 3.88 (s, 3H), 3.14-3.18 (m, 2H), 2.78 (t, J=7.09 Hz, 2H), 2.40-2.42 (m, 3H), 1.73-1.80 (m, 2H), 1.65-1.71 (m, 2H), 1.02 (s, 6H).

### Example 14. N-(5-(2-(4,4-difluoropiperidin-1-yl)acetamido)-2-methylpyridin-3-yl)-2-(1-methyl-1H-pyrazol-4-yl)pyrazolo[5,1-b]thiazole-7-carboxamide

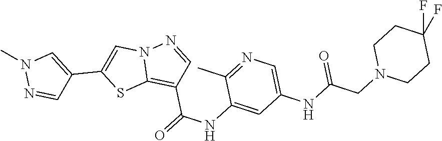 [SMILES: Cc1ncc(NC(=O)CN2CCC(F)(F)CC2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (120 mg, 0.23 mmol), 4,4-difluoropiperidine (34 mg, 0.28 mmol), potassium carbonate (97 mg, 0.70 mmol) in N,N-dimethylformamide (2 mL) was added sodium iodide (21 mg, 0.14 mmol). The mixture was stirred at 50° C. for 2 hours. The reaction mixture was concentrated under vacuum to give the crude product, which was purified by preparative high-performance liquid chromatography over Column: Boston Prime C18 150*30 mm*5 um to give the title compound N-(5-(2-(4,4-difluoropiperidin-1-yl)acetamido)-2-methylpyridin-3-yl)-2-(1-methyl-1H-pyrazol-4-yl)pyrazolo[5,1-b]thiazole-7-carboxamide (45 mg, 38%) as a white solid. LCMS (ESI): mass calcd. for C₂₃H₂₄F₂N₈O₂S, 514.1; m/z found, 515.2 [M+H]⁺. ¹H NMR (400 MHz, CHLOROFORM-d) δ 8.95 (s, 1H), 8.67 (d, J=2.21 Hz, 1H), 8.50 (d, J=2.21 Hz, 1H), 8.06 (s, 1H), 7.80 (s, 1H), 7.68 (s, 1H), 7.60 (s, 1H), 7.43 (s, 1H), 3.96 (s, 3H), 3.21 (s, 2H), 2.74 (br t, J=5.51 Hz, 4H), 2.56 (s, 3H), 2.01-2.16 (m, 4H).

### Example 15. 2-(1-methyl-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide

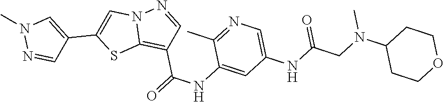 [SMILES: Cc1ncc(NC(=O)CN(C)C2CCOCC2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (100 mg, 0.15 mmol), N-methyltetrahydro-2H-pyran-4-amine (21 mg, 0.18 mmol), and potassium carbonate (63 mg, 0.45 mmol) in DMF (2 mL) was added sodium iodide (14 mg, 0.09 mmol). The resulting mixture was stirred at 50° C. for 2 h and then the reaction mixture was filtered and the filtrate was purified by preparative high-performance liquid chromatography over Column: Phenomenex Gemini-NX 80*40 mm*3 um to give the title compound: 2-(1-methyl-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide (38.1 mg, 49%) as a white solid. LCMS (ESI): mass calcd. for C₂₄H₂₈N₈O₃S, 508.6; m/z found, 509.3 [M+H]⁺. ¹H NMR (400 MHz, DMSO-d6) δ 9.85 (d, J=7.7 Hz, 2H), 8.59-8.52 (m, 2H), 8.47 (s, 1H), 8.17-8.13 (m, 2H), 7.84 (s, 1H), 3.84 (s, 3H), 3.30 (br s, 2H), 3.25 (br d, J=11.7 Hz, 2H), 3.21-3.19 (m, 2H), 2.62 (br d, J=4.2 Hz, 1H), 2.36 (s, 3H), 2.28 (s, 3H), 1.70 (br d, J=10.8 Hz, 2H), 1.49-1.35 (m, 2H).

### Example 16. N-(5-(2-(4-azaspiro[2.4]heptan-4-yl)acetamido)-2-methylpyridin-3-yl)-2-(1-methyl-1H-pyrazol-4-yl)pyrazolo[5,1-b]thiazole-7-carboxamide

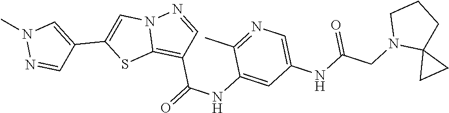 [SMILES: Cc1ncc(NC(=O)CN2CCCC23CC3)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.20 mmol), 4-azaspiro[2.4]heptane hydrochloride (33 mg, 0.24 mmol), and potassium carbonate (84 mg, 0.61 mmol) in DMF (2 mL) was added sodium iodide (18 mg, 0.12 mmol). The resulting mixture was stirred at 50° C. for 2 h and then the reaction mixture was filtered and the filtrate was purified by preparative high-performance liquid chromatography over Column: Phenomenex Gemini-NX 80*40 mm*3 um to give the title compound: N-(5-(2-(4-azaspiro[2.4]heptan-4-yl)acetamido)-2-methylpyridin-3-yl)-2-(1-methyl-1H-pyrazol-4-yl)pyrazolo[5,1-b]thiazole-7-carboxamide (57 mg, 54%) as a white solid. LCMS (ESI): mass calcd. for C₂₄H₂₆N₈O₂S, 490.6; m/z found, 491.3 [M+H]⁺. ¹H NMR (400 MHz, CHLOROFORM-d) δ 9.18 (s, 1H), 8.71 (d, J=2.2 Hz, 1H), 8.47 (d, J=2.2 Hz, 1H), 8.07 (s, 1H), 7.82 (s, 1H), 7.70 (s, 1H), 7.61 (s, 1H), 7.42 (s, 1H), 3.98 (s, 3H), 3.12 (s, 2H), 2.93 (t, J=7.1 Hz, 2H), 2.57 (s, 3H), 1.98 (br dd, J=6.6, 14.3 Hz, 2H), 1.86-1.76 (m, 2H), 0.77-0.68 (m, 2H), 0.57-0.47 (m, 2H).

### Example 17. 2-(1-methyl-1H-pyrazol-4-yl)-N-(2-methyl-5-(2-(1-methyl-4,5-dihydro-1H-pyrazolo[3,4-c]pyridin-6 (7H)-yl)acetamido)pyridin-3-yl)pyrazolo[5,1-b]thiazole-7-carboxamide

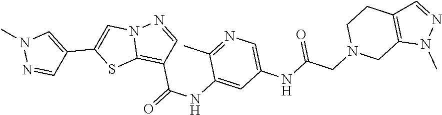

To a solution of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.20 mmol) and 1-methyl-4,5,6,7-tetrahydropyrazolo[3,4-c]pyridine dihydrochloride (60 mg, 0.29 mmol) in DMF (1.5 mL) was added K₂CO₃ (84 mg, 0.61 mmol) and NaI (18 mg, 0.12 mmol) at room-temperature. The mixture was stirred at 50° C. for 1.5 h. The mixture was filtered through Celite, rinsed with DMF (3 mL). The filtrate was concentrated to afford crude product. The crude product was purified by preparative high-performance liquid chromatography over Column: Phenomenex Gemini-NX 80*40 mm*3 um to give the title compound 2-(1-methyl-1H-pyrazol-4-yl)-N-(2-methyl-5-(2-(1-methyl-4,5-dihydro-1H-pyrazolo[3,4-c]pyridin-6 (7H)-yl)acetamido)pyridin-3-yl)pyrazolo[5,1-b]thiazole-7-carboxamide (55 mg, 100%) as a white solid. LCMS (ESI): mass calcd. for C₂₅H₂₆N₁₀O₂S, 530.6; m/z found, 531.3[M+H]⁺. ¹H NMR (400 MHz, DMSO-d6) δ 10.04 (s, 1H) 9.89 (s, 1H) 8.59 (s, 1H) 8.57 (s, 1H) 8.51 (s, 1H) 8.19 (s, 2H) 7.88 (s, 1H) 7.19 (s, 1H) 3.88 (s, 3H) 3.73 (s, 2H) 3.64 (s, 3H) 3.40-3.41 (m, 2H) 2.77-2.82 (m, 2H) 2.54-2.58 (m, 2H) 2.38-2.41 (m, 3H).

### Example 18. N-(5-(2-(2-oxa-6-azaspiro[3.4]octan-6-yl)acetamido)-2-methylpyridin-3-yl)-2-(1-methyl-1H-pyrazol-4-yl)pyrazolo[5,1-b]thiazole-7-carboxamide

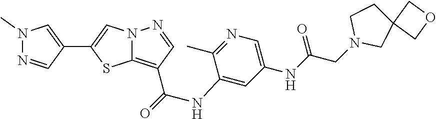 [SMILES: Cc1ncc(NC(=O)CN2CCC3(COC3)C2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (100 mg, 0.226 mmol), 2-oxa-6-azaspiro[3.4]octane (31 mg, 0.271 mmol), potassium carbonate (94 mg, 0.677 mmol) in N,N-dimethylformamide (2 mL) was added sodium iodide (20 mg, 0.135 mmol), The mixture was stirred at 50° C. for 2 hours. The reaction mixture was concentrated under vacuum to give the crude product, which was purified by preparative high-performance liquid chromatography over Column: Boston Prime C18 150*30 mm*5 um to give the title compound N-(5-(2-(2-oxa-6-azaspiro[3.4]octan-6-yl)acetamido)-2-methylpyridin-3-yl)-2-(1-methyl-1H-pyrazol-4-yl)pyrazolo[5,1-b]thiazole-7-carboxamide (68 mg, 60%) as a white solid. LCMS (ESI): mass calcd. for C₂₄H₂₆N₈O₃S, 506.1; m/z found, 507.2 [M+H]⁺. ¹H NMR (400 MHz, CHLOROFORM-d) δ 8.81 (s, 1H), 8.63 (s, 1H), 8.41 (s, 1H), 8.02 (s, 1H), 7.75 (s, 1H), 7.63 (s, 1H), 7.55 (s, 1H), 7.42 (s, 1H), 4.51-4.72 (m, 4H), 3.91 (s, 3H), 3.22 (s, 2H), 2.96 (s, 2H), 2.67 (t, J=6.95 Hz, 2H), 2.50 (s, 3H), 2.18 (t, J=7.06 Hz, 2H).

### Example 19. N-(5-(2-(2-oxa-7-azaspiro[4.4]nonan-7-yl)acetamido)-2-methylpyridin-3-yl)-2-(1-methyl-1H-pyrazol-4-yl)pyrazolo[5,1-b]thiazole-7-carboxamide

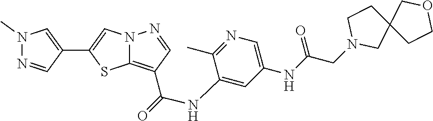 [SMILES: Cc1ncc(NC(=O)CN2CCC3(CCOC3)C2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (83 mg, 0.18 mmol), 2-oxa-7-azaspiro[4.4]nonane (28 mg, 0.22), and potassium carbonate (75 mg, 0.54 mmol) in DMF (3 mL) was added sodium iodide (20 mg, 0.14 mmol). The resulting mixture was stirred at 50° C. for 2 h and then the reaction mixture was filtered and the filtrate was purified by preparative high-performance liquid chromatography over Column: Phenomenex Gemini-NX 80*40 mm*3 um to give the title compound: N-(5-(2-(2-oxa-7-azaspiro[4.4]nonan-7-yl)acetamido)-2-methylpyridin-3-yl)-2-(1-methyl-1H-pyrazol-4-yl)pyrazolo[5,1-b]thiazole-7-carboxamide (39 mg, 41%) as a white solid. LCMS(ESI): mass calcd for C₂₅H₂₈N₈O₃S, 520.607; m/z found, 521.1 [M+H]+. ¹H NMR (400 MHz, METHANOL-d4) δ 8.56 (d, J=2.21 Hz, 1H), 8.37 (s, 1H), 8.23 (d, J=2.21 Hz, 1H), 8.19 (s, 1H), 8.00 (s, 1H), 7.79 (s, 1H), 3.92 (s, 3H), 3.84 (td, J=7.11, 4.74 Hz, 2H), 3.72 (d, J=8.38 Hz, 1H), 3.60 (d, J=8.38 Hz, 1H), 3.56 (s, 2H), 2.91-3.06 (m, 3H), 2.84 (d, J=9.92 Hz, 1H), 2.48 (s, 3H), 1.92-2.07 (m, 4H).

### Example 20. N-(5-(2-(2-oxa-5-azaspiro[3.4]octan-5-yl)acetamido)-2-methyl-pyridin-3-yl)-2-(1-methyl-1H-pyrazol-4-yl)pyrazolo[5,1-b]thiazole-7-carboxamide

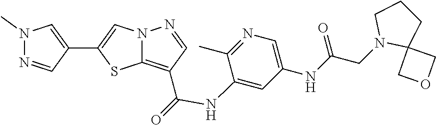 [SMILES: Cc1ncc(NC(=O)CN2CCCC23COC3)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (150 mg, 0.349 mmol), 2-oxa-5-azaspiro[3.4]octane oxalate (85 mg, 0.42 mmol), and potassium carbonate (145 mg, 1.047 mmol) in N,N-dimethylformamide (2 mL) was added sodium iodide (31 mg, 0.21 mmol), the mixture was stirred at 50° C. for 2 hours. The reaction mixture was concentrated under vacuum to give the crude product, which was purified by preparative high-performance liquid chromatography over Column: Xtimate C18 150*40 mm*5 um to give the title compound N-(5-(2-(2-oxa-5-azaspiro[3.4]octan-5-yl)acetamido)-2-methylpyridin-3-yl)-2-(1-methyl-1H-pyrazol-4-yl)pyrazolo[5,1-b]thiazole-7-carboxamide (43 mg, 24%) as a white solid. LCMS (ESI): mass calcd. for C₂₄H₂₆N₈O₃S, 506.2; m/z found, 507.2 [M+H]⁺. ¹H NMR (400 MHz, DMSO-d6) δ 9.90 (br d, J=14.55 Hz, 2H), 8.49-8.61 (m, 2H), 8.47 (s, 1H), 8.16 (s, 1H), 8.13 (d, J=1.98 Hz, 1H), 7.85 (s, 1H), 4.63 (d, J=6.62 Hz, 2H), 4.35 (d, J=6.61 Hz, 2H), 3.84 (s, 3H), 3.62 (s, 2H), 2.73 (t, J=6.95 Hz, 2H), 2.36 (s, 3H), 2.11 (t, J=7.50 Hz, 2H), 1.67 (quin, J=7.22 Hz, 2H).

### Example 21. N-(5-(2-(1-oxa-7-azaspiro[4.4]nonan-7-yl)acetamido)-2-methylpyridin-3-yl)-2-(1-methyl-1H-pyrazol-4-yl)pyrazolo[5,1-b]thiazole-7-carboxamide

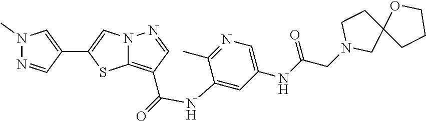 [SMILES: Cc1ncc(NC(=O)CN2CCC3(CCCO3)C2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (100 mg, 0.15 mmol), 1-oxa-7-azaspiro[4.4]nonane (23 mg, 0.18 mmol), and potassium carbonate (63 mg, 0.45 mmol) in DMF (2 mL) was added sodium iodide (14 mg, 0.09 mmol). The resulting mixture was stirred at 50° C. for 2 h and then the reaction mixture was filtered and the filtrate was purified by preparative high-performance liquid chromatography over Column: Phenomenex Gemini-NX 80*40 mm*3 um to give the title compound: N-(5-(2-(1-oxa-7-azaspiro[4.4]nonan-7-yl)acetamido)-2-methylpyridin-3-yl)-2-(1-methyl-1H-pyrazol-4-yl)pyrazolo[5,1-b]thiazole-7-carboxamide (58 mg, 85%) as a gray solid. LCMS (ESI): mass calcd. for C₂₅H₂₈N₈O₃S, 520.6; m/z found, 521.3 [M+H]⁺. ¹H NMR (400 MHz, DMSO-d6) δ 9.96-9.84 (m, 2H), 8.58-8.49 (m, 2H), 8.47 (s, 1H), 8.18-8.09 (m, 2H), 7.85 (s, 1H), 3.84 (s, 3H), 3.68-3.61 (m, 2H), 3.23 (s, 2H), 2.71-2.66 (m, 2H), 2.65-2.58 (m, 2H), 2.36 (s, 3H), 1.90-1.83 (m, 2H), 1.82-1.80 (m, 2H), 1.79-1.73 (m, 2H).

### Example 22. N-(5-(2-(3,3-dimethylazetidin-1-yl)acetamido)-2-methylpyridin-3-yl)-2-(1-methyl-1H-pyrazol-4-yl)pyrazolo[5,1-b]thiazole-7-carboxamide

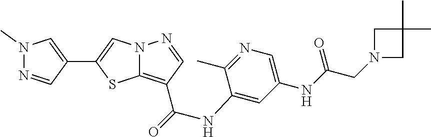

To a solution of N-(5-(2-chloroacetamido)-2-methylpyridin-3-yl)-2-(1-methyl-1H-pyrazol-4-yl)pyrazolo[5,1-b]thiazole-7-carboxamide (80 mg, 0.18 mmol), 3,3-dimethylazetidine hydrochloride (26 mg, 0.22 mmol), and potassium carbonate (75 mg, 0.54 mmol) in DMF (2 mL) was added sodium iodide (16 mg, 0.11 mmol). The resulting mixture was stirred at 50° C. for 2 h and then the reaction mixture was filtered over a pad of celite and concentrated under vacuum to give crude product, which was purified by silica gel column chromatography (eluent: ethyl acetate/dichloromethane/methanol=100:0:0 to 0:60:40) to give the title compound: N-(5-(2-(3,3-dimethylazetidin-1-yl)acetamido)-2-methylpyridin-3-yl)-2-(1-methyl-1H-pyrazol-4-yl)pyrazolo-[5,1-b]thiazole-7-carboxamide (60 mg, 65%) as a light red solid. LCMS (ESI): mass calcd. for C₂₃H₂₆N₈O₂S, 478.5; m/z found, 479.3 [M+H]⁺. ¹H NMR (400 MHz, DMSO-d6) δ 9.95 (br d, J=6.3 Hz, 1H), 9.89 (br s, 1H), 8.57 (s, 2H), 8.55 (br d, J=3.7 Hz, 1H), 8.20 (s, 1H), 8.13 (s, 1H), 7.88 (s, 1H), 3.88 (s, 3H), 3.24 (br s, 2H), 3.06 (s, 4H), 2.40 (s, 3H), 1.21 (s, 6H).

### Example 23. N-(5-(2-(3,4-dihydro-2,7-naphthyridin-2 (1H)-yl)acetamido)-2-methylpyridin-3-yl)-2-(1-methyl-1H-pyrazol-4-yl)pyrazolo[5,1-b]thiazole-7-carboxamide

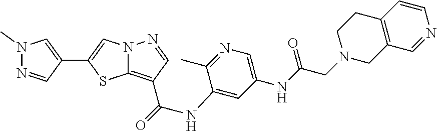 [SMILES: Cc1ncc(NC(=O)CN2CCc3ccncc3C2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.20 mmol) and 1,2,3,4-tetrahydro-2,7-naphthyridine; hydrochloride (42 mg, 0.24 mmol) in DMF (1.5 mL) was added K₂CO₃ (84.15 mg, 0.61 mmol) and NaI (18 mg, 0.12 mmol) at room-temperature. The mixture was stirred at 50° C. for 1.5 h. The mixture was filtered through Celite, rinsed with DMF (3 mL). The filtrate was concentrated to afford crude product. The crude product was purified by preparative high-performance liquid chromatography over Column: Phenomenex Gemini-NX 80*40 mm*3 um to give the title compound N-(5-(2-(3,4-dihydro-2,7-naphthyridin-2 (1H)-yl)acetamido)-2-methylpyridin-3-yl)-2-(1-methyl-1H-pyrazol-4-yl)pyrazolo[5,1-b]thiazole-7-carboxamide (53 mg, 100%) as a white solid. LCMS (ESI): mass calcd. for C₂₆H₂₅N₉O₂S, 527.6; m/z found, 528.3[M+H]⁺. ¹H NMR (400 MHz, DMSO-d6) δ 10.03 (br s, 2H), 8.54-8.64 (m, 2H), 8.49 (s, 1H), 8.31 (s, 1H), 8.29 (d, J=5.07 Hz, 1H), 8.19 (s, 2H), 7.88 (s, 1H), 7.16 (d, J=5.01 Hz, 1H), 3.88 (s, 3H), 3.77 (s, 2H), 3.39-3.39 (m, 2H), 2.82-2.93 (m, 4H), 2.39-2.45 (m, 3H).

### Example 24. N-(5-(2-(2-oxa-6-azaspiro[3.5]nonan-6-yl)acetamido)-2-methylpyridin-3-yl)-2-(1-methyl-1H-pyrazol-4-yl)pyrazolo[5,1-b]thiazole-7-carboxamide

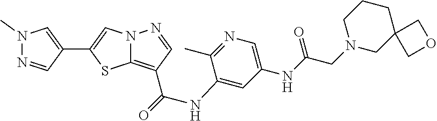 [SMILES: Cc1ncc(NC(=O)CN2CCCC3(COC3)C2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (80 mg, 0.17 mmol), 2-oxa-6-azaspiro[3.5]nonane (26 mg, 0.20 mmol), and potassium carbonate (70 mg, 0.51 mmol) in DMF (2 mL) was added sodium iodide (15 mg, 0.10 mmol) The resulting mixture was stirred at 50° C. for 2 h and then the reaction mixture was filtered and the filtrate was purified by preparative high-performance liquid chromatography over Column: Phenomenex Gemini-NX 80*40 mm*3 um to give the title compound: N-(5-(2-(2-oxa-6-azaspiro[3.5]nonan-6-yl)acetamido)-2-methylpyridin-3-yl)-2-(1-methyl-1H-pyrazol-4-yl)pyrazolo[5,1-b]thiazole-7-carboxamide (34 mg, 38%) as a white solid. LCMS (ESI): mass calcd. for C₂₅H₂₈N₈O₃S, 520.6; m/z found, 521.3 [M+H]⁺. ¹H NMR (400 MHz, CHLOROFORM-d) δ 8.97 (s, 1H), 8.56 (d, J=2.1 Hz, 1H), 8.47 (d, J=2.0 Hz, 1H), 8.10 (s, 1H), 7.80 (s, 1H), 7.68 (s, 1H), 7.62-7.56 (m, 2H), 4.49-4.39 (m, 4H), 3.97 (s, 3H), 3.15 (s, 2H), 2.75 (br s, 2H), 2.55 (s, 3H), 2.49 (br s, 2H), 1.69-1.55 (m, 4H).

### Example 25. N-(5-(2-(2-oxa-5-azaspiro[3.5]nonan-5-yl)acetamido)-2-methylpyridin-3-yl)-2-(1-methyl-1H-pyrazol-4-yl)pyrazolo[5,1-b]thiazole-7-carboxamide

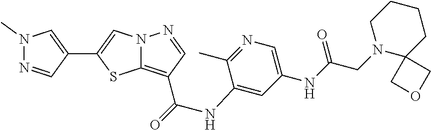 [SMILES: Cc1ncc(NC(=O)CN2CCCCC23COC3)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (100 mg, 0.23 mmol), 2-oxa-5-azaspiro[3.5]nonane oxalate (59 mg, 0.27 mmol), and potassium carbonate (94 mg, 0.68 mmol) in N,N-dimethylformamide (2 mL) was added sodium iodide (20 mg, 0.13 mmol), the resulting mixture was stirred at 50° C. for 2 hours. The reaction mixture was concentrated under vacuum to give the crude product, which was purified by preparative high-performance liquid chromatography over Column: Boston Prime C18 150*30 mm*5 um to give the title compound N-(5-(2-(2-oxa-5-azaspiro[3.5]nonan-5-yl)acetamido)-2-methylpyridin-3-yl)-2-(1-methyl-1H-pyrazol-4-yl)pyrazolo[5,1-b]thiazole-7-carboxamide (62 mg, 52%) as a white solid. LCMS (ESI): mass calcd. for C₂₅H₂₈N₈O₃S, 520.2; m/z found, 521.3 [M+H]⁺. ¹H NMR (400 MHz, CHLOROFORM-d) δ 9.31 (s, 1H), 8.64 (d, J=2.43 Hz, 1H), 8.50 (d, J=2.21 Hz, 1H), 8.07 (s, 1H), 7.80 (s, 1H), 7.68 (s, 1H), 7.60 (s, 1H), 7.46 (s, 1H), 4.59 (d, J=6.84 Hz, 2H), 4.41 (d, J=6.84 Hz, 2H), 3.96 (s, 3H), 3.46 (br s, 2H), 2.63 (br s, 2H), 2.56 (s, 3H), 1.89-2.00 (m, 2H), 1.56 (br s, 4H).

### Example 26. (R)-2-(1-methyl-1H-pyrazol-4-yl)-N-(2-methyl-5-(2-(2-methyl-pyrrolidin-1-yl)acetamido)pyridin-3-yl)pyrazolo[5,1-b]thiazole-7-carboxamide

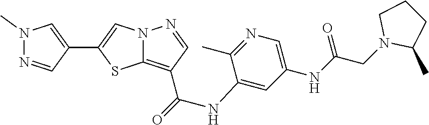

To a solution of N-(5-(2-chloroacetamido)-2-methylpyridin-3-yl)-2-(1-methyl-1H-pyrazol-4-yl)pyrazolo[5,1-b]thiazole-7-carboxamide (80 mg, 0.17 mmol), (R)-2-methylpyrrolidine hydrochloride (25 mg, 0.2 mmol), and potassium carbonate (70 mg, 0.5 mmol) in DMF (2 mL) was added sodium iodide (7.5 mg, 0.05 mmol). The resulting mixture was stirred at 50° C. for 2 h and then the reaction mixture was filtered and the filtrate was purified by preparative high-performance liquid chromatography over Column: Phenomenex Gemini-NX 80*40 mm*3 um to give the title compound: (R)-2-(1-methyl-1H-pyrazol-4-yl)-N-(2-methyl-5-(2-(2-methylpyrrolidin-1-yl)acetamido) pyridin-3-yl)pyrazolo[5,1-b]thiazole-7-carboxamide (31 mg, 38%) as a white solid. LCMS(ESI): mass calcd. for C₂₃H₂₆N₈O₂S, 478.57; m/z found, 479.2 [M+H]+. ¹H NMR (400 MHz, DMSO-d6) δ 9.82-10.01 (m, 2H), 8.46-8.70 (m, 3H), 8.18 (br d, J=8.46 Hz, 2H), 7.88 (s, 1H), 3.88 (s, 3H), 3.11 (br dd, J=8.34, 5.60 Hz, 1H), 3.03 (br d, J=15.62 Hz, 1H), 3.00-3.07 (m, 1H), 2.53-2.56 (m, 1H), 2.41 (s, 3H), 2.33 (q, J=8.50 Hz, 1H), 1.86-1.97 (m, 1H), 1.61-1.81 (m, 2H), 1.33-1.46 (m, 1H), 1.07 (d, J=5.96 Hz, 3H).

### Example 27. (S)-2-(1-methyl-1H-pyrazol-4-yl)-N-(2-methyl-5-(2-(2-methyl-pyrrolidin-1-yl)acetamido)pyridin-3-yl)pyrazolo[5,1-b]thiazole-7-carboxamide

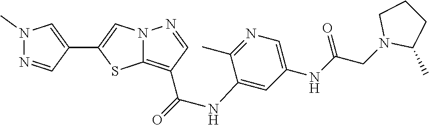

To a solution of N-(5-(2-chloroacetamido)-2-methylpyridin-3-yl)-2-(1-methyl-1H-pyrazol-4-yl)pyrazolo[5,1-b]thiazole-7-carboxamide (100 mg, 0.21 mmol), (S)-2-methylpyrrolidine (21.5 mg, 0.25 mmol), and potassium carbonate (87.2 mg, 0.63 mmol) in DMF (2 mL) was added sodium iodide (18.9 mg, 0.13 mmol). The resulting mixture was stirred at 50° C. for 2 h and then the reaction mixture was filtered and the filtrate was purified by preparative high-performance liquid chromatography over Column: Phenomenex Gemini-NX 80*40 mm*3 um to give the title compound: (S)-2-(1-methyl-1H-pyrazol-4-yl)-N-(2-methyl-5-(2-(2-methylpyrrolidin-1-yl)acetamido) pyridin-3-yl)pyrazolo[5,1-b]thiazole-7-carboxamide (29.1 mg, 29%) as a white solid. LCMS(ESI): mass calcd. for C₂₃H₂₆N₈O₂S, 478.57; m/z found, 479.3 [M+H]+. ¹H NMR (400 MHz, CHLOROFORM-d) δ 9.14 (br s, 2H), 8.61 (d, J=2.15 Hz, 1H), 8.06 (s, 1H), 7.74 (s, 1H), 7.62 (s, 1H), 7.49-7.59 (m, 2H), 3.90 (s, 3H), 3.37 (d, J=16.93 Hz, 1H), 3.00-3.16 (m, 2H), 2.48-2.59 (m, 4H), 2.33 (q, J=8.82 Hz, 1H), 1.91-1.97 (m, 1H), 1.73-1.85 (m, 2H), 1.40-1.48 (m, 1H), 1.06 (d, J=6.08 Hz, 3H).

### Example 28. N-(5-(2-(3,3-dimethylpyrrolidin-1-yl)acetamido)-2-methylpyridin-3-yl)-2-(1-(2-hydroxyethyl)-1H-pyrazol-4-yl)pyrazolo[5,1-b]thiazole-7-carboxamide

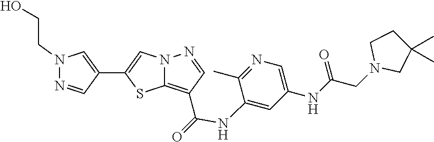 [SMILES: Cc1ncc(NC(=O)CN2CCC(C)(C)C2)cc1NC(=O)c1cnn2cc(-c3cnn(CCO)c3)sc12]

### Step a: N-(5-amino-2-methylpyridin-3-yl)-2-bromopyrazolo[5,1-b]thiazole-7-carboxamide

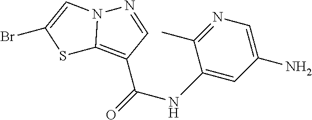 [SMILES: Cc1ncc(N)cc1NC(=O)c1cnn2cc(Br)sc12]

The solution of tert-butyl (5-(2-bromopyrazolo[5,1-b]thiazole-7-carboxamido)-6-methylpyridin-3-yl)carbamate (3 g, 5.86 mmol) in HCl/dioxane (30 mL) was stirred at 30° C. for 12 h. The mixture was concentrated under vacuum to give desired product N-(5-amino-2-methylpyridin-3-yl)-2-bromopyrazolo[5,1-b]thiazole-7-carboxamide (2.64 g, 100%) as white solid. LCMS (ESI): mass calcd. for C₁₂H₁₀BrN₅OS, 352.2; m/z found, 353.8 [M+H]+.

### Step b: 2-bromo-N-(5-(2-chloroacetamido)-2-methylpyridin-3-yl)pyrazolo[5,1-b]thiazole-7-carboxamide

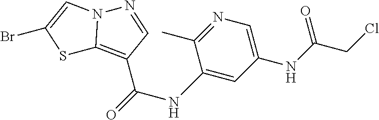 [SMILES: Cc1ncc(NC(=O)CCl)cc1NC(=O)c1cnn2cc(Br)sc12]

To a solution of N-(5-amino-2-methylpyridin-3-yl)-2-bromopyrazolo[5,1-b]thiazole-7-carboxamide (1 g, 2.68 mmol) and sodium bicarbonate (0.675 g, 8.04 mmol) in N,N-dimethylformamide (6 mL) was added 2-chloroacetyl chloride (0.256 ml, 1.42 mmol) at room-temperature. The reaction mixture was stirred at 40° C. for 16 h before cooling to room-temperature. The mixture was adjusted to pH=7-8 with NaHCO₃ (aq,). The mixture was filtered and washed with water (10 mL×3). The solid was evaporated under vacuum to give desired product 2-bromo-N-(5-(2-chloroacetamido)-2-methylpyridin-3-yl)pyrazolo[5,1-b]thiazole-7-carboxamide (900 mg, 78%) as white solid. LCMS (ESI): mass calcd. for C₁₄H₁₁BrClN₅O₂S, 428.6; m/z found, 429.8 [M+H]+.

### Step c: 2-bromo-N-(5-(2-(3,3-dimethylpyrrolidin-1-yl)acetamido)-2-methylpyridin-3-yl)pyrazolo[5,1-b]thiazole-7-carboxamide

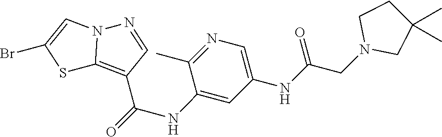 [SMILES: Cc1ncc(NC(=O)CN2CCC(C)(C)C2)cc1NC(=O)c1cnn2cc(Br)sc12]

To a solution of 2-bromo-N-(5-(2-chloroacetamido)-2-methylpyridin-3-yl)pyrazolo[5,1-b]thiazole-7-carboxamide (200 mg, 0.47 mmol), potassium carbonate (193 mg, 1.4 mmol) and sodium iodide (42 mg, 0.28 mmol) in N,N-dimethylformamide (6 mL) was added 3,3-dimethylpyrrolidine (76 mg, 0.56 mmol) at room-temperature. The resulting mixture was stirred at 60° C. for 2 h before cooling to room temperature. The resulting mixture was quenched with water (5 ml) and extracted with ethyl acetate (10 ml*3). The organic extracts were dried over anhydrous Na₂SO₄ and filtered. The filtration was concentrated under reduced pressure to give crude product, which was purified by column chromatography over silica gel (eluent: petroleum ether:ethyl acetate=0:1) to give the title compound 2-bromo-N-(5-(2-(3,3-dimethylpyrrolidin-1-yl)acetamido)-2-methylpyridin-3-yl)pyrazolo[5,1-b]thiazole-7-carboxamide (100 mg, 44%) as a white solid. LCMS (ESI): mass calcd. for C₂₀H₂₃BrN₆O₂S, 491.4; m/z found, 491.0 [M+H]+.

### Step e: N-(5-(2-(3,3-dimethylpyrrolidin-1-yl)acetamido)-2-methylpyridin-3-yl)-2-(1-(2-hydroxyethyl)-1H-pyrazol-4-yl)pyrazolo[5,1-b]thiazole-7-carboxamide

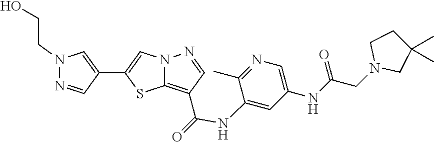 [SMILES: Cc1ncc(NC(=O)CN2CCC(C)(C)C2)cc1NC(=O)c1cnn2cc(-c3cnn(CCO)c3)sc12]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (100 mg, 0.20 mmol) and 2-(4-(4,4,5,5-tetramethyl-1,3,2-dioxaborolan-2-yl)-1H-pyrazol-1-yl)ethanol (58 mg, 0.36 mmol) in 1,4-dioxane (4 mL) and H₂O (1 mL) was added [1,1-bis(diphenylphosphino)ferrocene]palladium(II) chloride dichloromethane complex (33 mg, 0.04 mmol) and K₃PO₄ (130 mg, 0.61 mmol) under N₂. The resulting mixture was stirred at 90° C. for 3 h before cooled to 25° C. The reaction mixture was diluted with ethyl acetate (10 mL) and filtered over celite. The filtration was concentrated to the crude product, which was treated with dichloromethane (10 mL) and water (10 mL). The organic phase was washed with 3 M HCl aqueous solution, dried over anhydrous Na₂SO₄ and filtered. The filtration was concentrated under reduced pressure to give the crude product, which was purified by preparative high-performance liquid chromatography over Column: Boston Prime C18 150*30 mm*5 um to give the title compound N-(5-(2-(3,3-dimethylpyrrolidin-1-yl)acetamido)-2-methylpyridin-3-yl)-2-(1-(2-hydroxyethyl)-1H-pyrazol-4-yl)pyrazolo[5,1-b]thiazole-7-carboxamide (23 mg, 22%) as a yellow solid. LCMS (ESI): mass calcd. for C₂₅H₃₀N₈O₃S, 522.62; m/z found, 523.2 [M+H]⁺. ¹H NMR (400 MHz, METHANOL-d4) δ 8.61 (d, J=2.3 Hz, 1H), 8.41 (s, 1H), 8.26 (s, 2H), 8.11 (s, 1H), 7.89 (s, 1H), 4.30 (t, J=5.3 Hz, 2H), 3.94 (t, J=5.4 Hz, 2H), 3.38 (s, 2H), 2.87 (t, J=7.0 Hz, 2H), 2.56 (s, 2H), 2.52 (s, 3H), 1.72 (t, J=7.0 Hz, 2H), 1.17 (s, 6H).

### Example 29. N-(5-(2-(5-azaspiro[3.4]octan-5-yl)acetamido)-2-methylpyridin-3-yl)-2-(1-(2-hydroxyethyl)-1H-pyrazol-4-yl)pyrazolo[5,1-b]thiazole-7-carboxamide

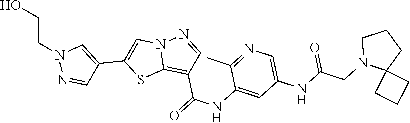 [SMILES: Cc1ncc(NC(=O)CN2CCCC23CCC3)cc1NC(=O)c1cnn2cc(-c3cnn(CCO)c3)sc12]

### Step a: N-(5-(2-(5-azaspiro[3.4]octan-5-yl)acetamido)-2-methylpyridin-3-yl)-2-bromopyrazolo[5,1-b]thiazole-7-carboxamide

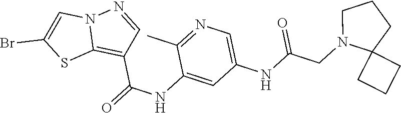 [SMILES: Cc1ncc(NC(=O)CN2CCCC23CCC3)cc1NC(=O)c1cnn2cc(Br)sc12]

To a solution of 2-bromo-N-(5-(2-chloroacetamido)-2-methylpyridin-3-yl)pyrazolo[5,1-b]thiazole-7-carboxamide (250 mg, 0.53 mmol), 5-azaspiro[3.4]octane (71 mg, 0.64 mmol), and potassium carbonate (221 mg, 1.60 mmol) in DMF (4 mL) was added sodium iodide (48 mg, 0.32 mmol). The resulting mixture was stirred at 50° C. for 2 h and then the reaction mixture was filtered and the filtrate was purified by silica gel column chromatography (eluent: petroleum ether/ethyl acetate=100:0 to 0:100) to give the title compound: N-(5-(2-(5-azaspiro[3.4]octan-5-yl)acetamido)-2-methylpyridin-3-yl)-2-bromopyrazolo[5,1-b]thiazole-7-carboxamide (190 mg, 62%) as a yellow solid. LCMS(ESI): mass calcd. for C₂₁H₂₃BrN₆O₂S, 503.4; m/z found, 505.1 [M+H]+.

### Step b: N-(5-((1-(2-(1-(2-hydroxyethyl)-1H-pyrazol-4-yl)pyrazolo[5,1-b]thiazol-7-yl)vinyl)amino)-6-methylpyridin-3-yl)-2-(5-azaspiro[3.4]octan-5-yl)acetamide

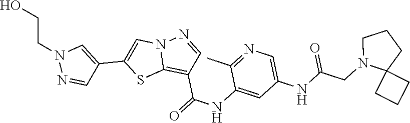 [SMILES: Cc1ncc(NC(=O)CN2CCCC23CCC3)cc1NC(=O)c1cnn2cc(-c3cnn(CCO)c3)sc12]

To a solution of N-(5-(2-(5-azaspiro[3.4]octan-5-yl)acetamido)-2-methylpyridin-3-yl)-2-bromopyrazolo[5,1-b]thiazole-7-carboxamide (170 mg, 0.30 mmol), 2-(4-(4,4,5,5-tetramethyl-1,3,2-dioxaborolan-2-yl)-1H-pyrazol-1-yl)ethanol (85 mg, 0.36 mmol) and potassium phosphate (189 mg, 0.89 mmol) in dioxane/H₂O=4:1 (5 mL) was added 1,1-bis(diphenylphosphino)ferrocene-palladium(II)dichloride dichloromethane complex (48 mg, 0.06 mmol) and the system was degassed by applying alternating N2 atmosphere. The mixture was stirred at 95° C. for overnight and then the reaction mixture was cooled to r.t and evaporated in vacuum to afford crude product, which was purified by silica gel column chromatography (eluent: petroleum ether/ethyl acetate/methanol=100:0:0 to 0:70:30) to give pure product. Then the pure product was purified by preparative high-performance liquid chromatography over Column: Xtimate C18 150*40 mm*5 um to give the title compound: N-(5-((1-(2-(1-(2-hydroxyethyl)-1H-pyrazol-4-yl)pyrazolo[5,1-b]thiazol-7-yl)vinyl)amino)-6-methylpyridin-3-yl)-2-(5-azaspiro[3.4]octan-5-yl)acetamide (59 mg, 37%) as a gray solid. LCMS(ESI): mass calcd. for C₂₆H₃₀N₈O₃S, 534.6, m/z found, 535.2 [M+H]+. ¹H NMR (400 MHz, CHLOROFORM-d) δ 9.15 (br s, 1H), 8.69 (s, 1H), 8.48 (br s, 1H), 8.07 (br s, 1H), 7.82 (s, 1H), 7.71 (br d, J=6.6 Hz, 2H), 7.43 (br s, 1H), 7.25 (s, 1H), 4.31 (br d, J=4.0 Hz, 2H), 4.07 (br s, 2H), 3.34 (s, 2H), 2.79 (br d, J=7.1 Hz, 2H), 2.56 (s, 3H), 2.15-1.97 (m, 6H), 1.88-1.75 (m, 4H).

### Example 30. N-(5-(2-(6-azaspiro[3.4]octan-6-yl)acetamido)-2-methylpyridin-3-yl)-2-(1-(2-hydroxyethyl)-1H-pyrazol-4-yl)pyrazolo[5,1-b]thiazole-7-carboxamide

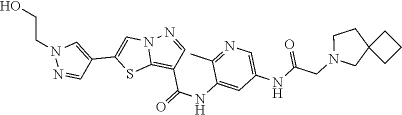 [SMILES: Cc1ncc(NC(=O)CN2CCC3(CCC3)C2)cc1NC(=O)c1cnn2cc(-c3cnn(CCO)c3)sc12]

### Step a: N-(5-(2-(6-azaspiro[3.4]octan-6-yl)acetamido)-2-methylpyridin-3-yl)-2-bromopyrazolo[5,1-b]thiazole-7-carboxamide

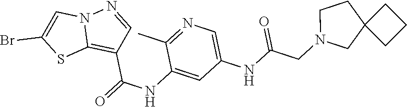 [SMILES: Cc1ncc(NC(=O)CN2CCC3(CCC3)C2)cc1NC(=O)c1cnn2cc(Br)sc12]

To a solution of 2-bromo-N-(5-(2-chloroacetamido)-2-methylpyridin-3-yl)pyrazolo[5,1-b]thiazole-7-carboxamide (300 mg, 0.64 mmol), 6-azaspiro[3.4]octane (85 mg, 0.77 mmol), and cesium carbonate (625 mg, 1.92 mmol) in DMF (5 mL) was added sodium iodide (58 mg, 0.38 mmol). The resulting mixture was stirred at 50° C. for 2 h and then the reaction mixture was filtered and the filtrate was purified by silica gel column chromatography (eluent: petroleum ether/ethyl acetate=100:0 to 0:100) to give the title compound: N-(5-(2-(6-azaspiro[3.4]octan-6-yl)acetamido)-2-methylpyridin-3-yl)-2-bromopyrazolo[5,1-b]thiazole-7-carboxamide (212 mg, 65%) as a light white solid. LCMS(ESI): mass calcd. for C₂₁H₂₃BrN₆O₂S, 503.4; m/z found, 505.1 [M+H]+.

### Step b: N-(5-(2-(6-azaspiro[3.4]octan-6-yl)acetamido)-2-methylpyridin-3-yl)-2-(1-(2-hydroxyethyl)-1H-pyrazol-4-yl)pyrazolo[5,1-b]thiazole-7-carboxamide

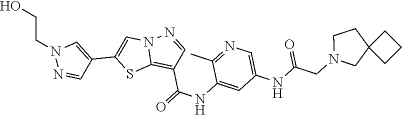 [SMILES: Cc1ncc(NC(=O)CN2CCC3(CCC3)C2)cc1NC(=O)c1cnn2cc(-c3cnn(CCO)c3)sc12]

To a solution of N-(5-(2-(6-azaspiro[3.4]octan-6-yl)acetamido)-2-methylpyridin-3-yl)-2-bromopyrazolo[5,1-b]thiazole-7-carboxamide (190 mg, 0.38 mmol), 2-(4-(4,4,5,5-tetramethyl-1,3,2-dioxaborolan-2-yl)-1H-pyrazol-1-yl)ethanol (108 mg, 0.45 mmol) and potassium phosphate (240 mg, 1.13 mmol) in dioxane/H₂O=4:1 (6.25 mL) was added 1,1-bis(diphenylphosphino)ferrocene-palladium(II)dichloride dichloromethane complex (62 mg, 0.08 mmol) and the system was degassed by applying alternating N2 atmosphere. The mixture was stirred at 95° C. for overnight and then the reaction mixture was cooled to r.t and evaporated in vacuum to afford crude product, which was purified by silica gel column chromatography (eluent: petroleum ether/ethyl acetate/methanol=100:0:0 to 0:70:30) to give pure product. Then the pure product was purified by preparative high-performance liquid chromatography over Column: Phenomenex Gemini-NX C18 75*30 mm*3 um to give the title compound: N-(5-(2-(6-azaspiro[3.4]octan-6-yl)acetamido)-2-methylpyridin-3-yl)-2-(1-(2-hydroxyethyl)-1H-pyrazol-4-yl)pyrazolo[5,1-b]thiazole-7-carboxamide (48 mg, 23%) as a white solid. LCMS(ESI): mass calcd. for C₂₆H₃₀N₈O₃S, 534.6, m/z found, 535.3 [M+H]+. ¹H NMR (400 MHz, CHLOROFORM-d) δ 9.18 (br s, 1H), 8.60 (br s, 1H), 8.53 (br s, 1H), 8.06 (br s, 1H), 7.81 (d, J=5.1 Hz, 1H), 7.73-7.68 (m, 2H), 7.44 (br s, 1H), 7.25 (br s, 1H), 4.31 (br d, J=4.6 Hz, 2H), 4.06 (br d, J=4.2 Hz, 2H), 3.27 (br d, J=5.3 Hz, 2H), 2.75 (br s, 4H), 2.56 (br d, J=4.9 Hz, 3H), 2.04-1.91 (m, 6H), 1.85 (br s, 2H).

### Example 31. N-(5-(2-(6-azaspiro[2.5]octan-6-yl)acetamido)-2-methylpyridin-3-yl)-2-(1-(2-hydroxyethyl)-1H-pyrazol-4-yl)pyrazolo[5,1-b]thiazole-7-carboxamide

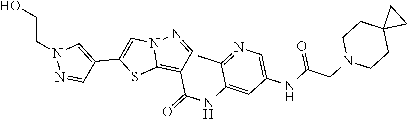 [SMILES: Cc1ncc(NC(=O)CN2CCC3(CC2)CC3)cc1NC(=O)c1cnn2cc(-c3cnn(CCO)c3)sc12]

### Step a: N-(5-(2-(6-azaspiro[2.5]octan-6-yl)acetamido)-2-methylpyridin-3-yl)-2-bromopyrazolo[5,1-b]thiazole-7-carboxamide

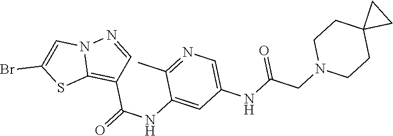 [SMILES: Cc1ncc(NC(=O)CN2CCC3(CC2)CC3)cc1NC(=O)c1cnn2cc(Br)sc12]

To a solution of 2-bromo-N-(5-(2-chloroacetamido)-2-methylpyridin-3-yl)pyrazolo[5,1-b]thiazole-7-carboxamide (300 mg, 0.7 mmol), 6-azaspiro[2.5]octane (93 mg, 0.8 mmol), and cesium carbonate (684 mg, 2.1 mmol) in DMF (5 mL) was added sodium iodide (63 mg, 0.42 mmol). The resulting mixture was stirred at 50° C. for 2 h. The resulting mixture was quenched with water (15 ml) and extracted with ethyl acetate (30 ml*3). The organic extracts were dried over anhydrous Na₂SO₄ and filtered. The filtration was concentrated under reduced pressure to give crude product, which was purified by column chromatography over silica gel (eluent: petroleum ether:ethyl acetate=4:1) to give the title compound: N-(5-(2-(6-azaspiro[2.5]octan-6-yl)acetamido)-2-methylpyridin-3-yl)-2-bromopyrazolo[5,1-b]thiazole-7-carboxamide (150 mg, 29%). LCMS (ESI): mass calcd for C₂₁H₂₃BrN₆O₂S, 503.415; m/z found, 504.2 [M+H]+.

### Step b: N-(5-(2-(6-azaspiro[2.5]octan-6-yl)acetamido)-2-methylpyridin-3-yl)-2-(1-(2-hydroxyethyl)-1H-pyrazol-4-yl)pyrazolo[5,1-b]thiazole-7-carboxamide

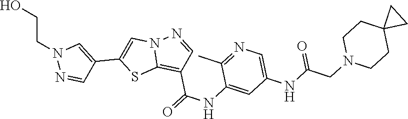 [SMILES: Cc1ncc(NC(=O)CN2CCC3(CC2)CC3)cc1NC(=O)c1cnn2cc(-c3cnn(CCO)c3)sc12]

To a solution of N-(5-(2-(6-azaspiro[2.5]octan-6-yl)acetamido)-2-methylpyridin-3-yl)-2-bromopyrazolo[5,1-b]thiazole-7-carboxamide (150 mg, 0.21 mmol), 2-(4-(4,4,5,5-tetramethyl-1,3,2-dioxaborolan-2-yl)-1H-pyrazol-1-yl)ethanol (60 mg, 0.25 mmol), and potassium phosphate (133 mg, 0.63 mmol) in dioxane/H₂O=4:1 (5 mL) was added 1,1-bis(diphenylphosphino)ferrocene-palladium(II)dichloride dichloromethane complex (34 mg, 0.04 mmol) and the system was degassed by applying alternating N₂ atmosphere. The mixture was stirred at 100° C. for overnight and then the reaction mixture was filtered and the filtrate was purified by preparative high-performance liquid chromatography over Column: Phenomenex Gemini-NX 80*40 mm*3 um to give the title compound: N-(5-(2-(6-azaspiro[2.5]octan-6-yl)acetamido)-2-methylpyridin-3-yl)-2-(1-(2-hydroxyethyl)-1H-pyrazol-4-yl)pyrazolo[5,1-b]thiazole-7-carboxamide as a white solid (33 mg, 29%). LCMS(ESI): mass calcd for C₂₆H₃₀N₈O₃S, 534.633, m/z found, 535.3 [M+H]+. ¹H NMR (400 MHz, METHANOL-d4) δ 8.61 (d, J=1.79 Hz, 1H), 8.41 (s, 1H), 8.27 (d, J=2.15 Hz, 1H), 8.24 (s, 1H), 8.10 (s, 1H), 7.87 (s, 1H), 4.30 (t, J=5.25 Hz, 2H), 3.94 (t, J=5.25 Hz, 2H), 3.55 (s, 2H), 2.91 (br s, 4H), 2.53 (s, 3H), 1.61 (br s, 4H), 0.40 (s, 4H).

### Example 32. N-(5-(2-(5-azaspiro[2.4]heptan-5-yl)acetamido)-2-methylpyridin-3-yl)-2-(1-(2-hydroxyethyl)-1H-pyrazol-4-yl)pyrazolo[5,1-b]thiazole-7-carboxamide

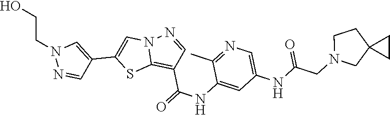 [SMILES: Cc1ncc(NC(=O)CN2CCC3(CC3)C2)cc1NC(=O)c1cnn2cc(-c3cnn(CCO)c3)sc12]

### Step a: N-(5-(2-(5-azaspiro[2.4]heptan-5-yl)acetamido)-2-methylpyridin-3-yl)-2-bromopyrazolo[5,1-b]thiazole-7-carboxamide

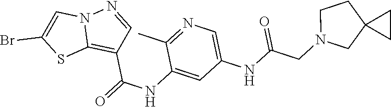 [SMILES: Cc1ncc(NC(=O)CN2CCC3(CC3)C2)cc1NC(=O)c1cnn2cc(Br)sc12]

To a solution of 2-bromo-N-(5-(2-chloroacetamido)-2-methylpyridin-3-yl)pyrazolo[5,1-b]thiazole-7-carboxamide (200 mg, 0.47 mmol), 5-azaspiro[2.4]heptane (54 mg, 0.56 mmol), and cesium carbonate (456.02 mg, 1.4 mmol) in DMF (3 mL) was added sodium iodide (42 mg, 0.28 mmol). The resulting mixture was stirred at 50° C. for 2 h. The resulting mixture was quenched with water (15 ml) and extracted with ethyl acetate (30 ml*3). The organic extracts were dried over anhydrous Na₂SO₄ and filtered. The filtration was concentrated under reduced pressure to give crude product, which was purified by column chromatography over silica gel (eluent: petroleum ether:ethyl acetate=4:1) to give the title compound: N-(5-(2-(5-azaspiro[2.4]heptan-5-yl)acetamido)-2-methylpyridin-3-yl)-2-bromopyrazolo[5,1-b]thiazole-7-carboxamide (150 mg, 55%) as a yellow solid. LCMS (ESI): mass calcd for C₂₀H₂₁BrN₆O₂S, 489.389; m/z found, 491.1 [M+H]+.

### Step b: N-(5-(2-(5-azaspiro[2.4]heptan-5-yl)acetamido)-2-methylpyridin-3-yl)-2-(1-(2-hydroxyethyl)-1H-pyrazol-4-yl)pyrazolo[5,1-b]thiazole-7-carboxamide

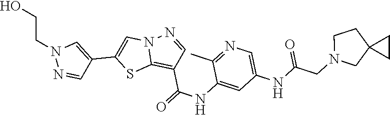 [SMILES: Cc1ncc(NC(=O)CN2CCC3(CC3)C2)cc1NC(=O)c1cnn2cc(-c3cnn(CCO)c3)sc12]

To a solution of N-(5-(2-(5-azaspiro[2.4]heptan-5-yl)acetamido)-2-methylpyridin-3-yl)-2-bromopyrazolo[5,1-b]thiazole-7-carboxamide (150 mg, 0.26 mmol), 2-(4-(4,4,5,5-tetramethyl-1,3,2-dioxaborolan-2-yl)-1H-pyrazol-1-yl)ethanol (74 mg, 0.31 mmol), and potassium phosphate (166 mg, 0.78 mmol) in dioxane/H₂O=4:1 (5 mL) was added 1,1-bis(diphenylphosphino)ferrocene-palladium(II)dichloride dichloromethane complex (63.73 mg, 0.08 mmol) and the system was degassed by applying alternating N₂ atmosphere. The mixture was stirred at 100° C. for overnight, and then the reaction mixture was filtered and the filtrate was purified by preparative high-performance liquid chromatography over Column: Phenomenex Gemini-NX 80*40 mm*3 um to give the title compound: N-(5-(2-(5-azaspiro[2.4]heptan-5-yl)acetamido)-2-methylpyridin-3-yl)-2-(1-(2-hydroxyethyl)-1H-pyrazol-4-yl)pyrazolo[5,1-b]thiazole-7-carboxamide as a white solid (22 mg, 15%). LCMS(ESI): mass calcd for C₂₅H₂₈N₈O₃S, 520.61, m/z found, 521.3 [M+H]+. ¹H NMR (400 MHz, METHANOL-d4) δ 8.61 (d, J=2.15 Hz, 1H), 8.41 (s, 1H), 8.22-8.29 (m, 2H), 8.10 (s, 1H), 7.88 (s, 1H), 4.30 (t, J=5.25 Hz, 2H), 3.94 (t, J=5.25 Hz, 2H), 3.44 (s, 2H), 2.97 (t, J=6.97 Hz, 2H), 2.75 (s, 2H), 2.52 (s, 3H), 1.86-1.96 (m, 2H), 0.54-0.69 (m, 4H).

### Example 33. (R)-2-(1-(2-hydroxyethyl)-1H-pyrazol-4-yl)-N-(2-methyl-5-(2-(2-methyl-piperidin-1-yl)acetamido)pyridin-3-yl)pyrazolo[5,1-b]thiazole-7-carboxamide

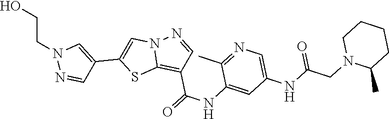 [SMILES: Cc1ncc(NC(=O)CN2CCCC[C@H]2C)cc1NC(=O)c1cnn2cc(-c3cnn(CCO)c3)sc12]

### Step a. (RS)-2-bromo-N-(2-methyl-5-(2-(2-methylpiperidin-1-yl)acetamido)pyridin-3-yl)pyrazolo[5,1-b]thiazole-7-carboxamide

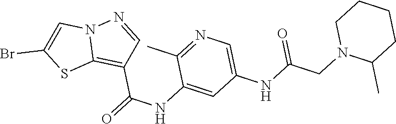 [SMILES: Cc1ncc(NC(=O)CN2CCCCC2C)cc1NC(=O)c1cnn2cc(Br)sc12]

To a solution of 2-bromo-N-(5-(2-chloroacetamido)-2-methylpyridin-3-yl)pyrazolo[5,1-b]thiazole-7-carboxamide (350 mg, 0.8 mmol), (RS)-2-methylpiperidine (97 mg, 0.98 mmol), and cesium carbonate (798 mg, 2.44 mmol) in DMF (3 mL) was added sodium iodide (73 mg, 0.49 mmol). The resulting mixture was stirred at 50° C. for 2 h. The resulting mixture was quenched with water (15 ml) and extracted with ethyl acetate (30 ml*3). The organic extracts were dried over anhydrous Na₂SO₄ and filtered. The filtration was concentrated under reduced pressure to give crude product, which was purified by column chromatography over silica gel (eluent: petroleum ether:ethyl acetate=4:1) to give the title compound: (RS)-2-bromo-N-(2-methyl-5-(2-(2-methylpiperidin-1-yl)acetamido)pyridin-3-yl)pyrazolo[5,1-b]thiazole-7-carboxamide (300 mg, 32%). LCMS (ESI): mass calcd for C₂₀H₂₃BrN₆O₂S, 491.405; m/z found, 491.1 [M+H]+.

### Step b: (RS)-2-(1-(2-hydroxyethyl)-1H-pyrazol-4-yl)-N-(2-methyl-5-(2-(2-methylpiperidin-1-yl)acetamido)pyridin-3-yl)pyrazolo[5,1-b]thiazole-7-carboxamide

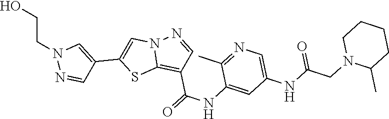 [SMILES: Cc1ncc(NC(=O)CN2CCCCC2C)cc1NC(=O)c1cnn2cc(-c3cnn(CCO)c3)sc12]

To a solution of (RS)-2-bromo-N-(2-methyl-5-(2-(2-methylpiperidin-1-yl)acetamido)pyridin-3-yl)pyrazolo[5,1-b]thiazole-7-carboxamide (130 mg, 0.26 mmol), 2-(4-(4,4,5,5-tetramethyl-1,3,2-dioxaborolan-2-yl)-1H-pyrazol-1-yl)ethanol (76 mg, 0.32 mmol), and potassium phosphate (169 mg, 0.8 mmol) in dioxane/H₂O=4:1 (5 mL) was added 1,1-bis(diphenylphosphino)ferrocene-palladium(II)dichloride dichloromethane complex (65 mg, 0.08 mmol) and the system was degassed by applying alternating N₂ atmosphere. The mixture was stirred at 100° C. for overnight, and then the reaction mixture was filtered and the filtrate was purified by preparative high-performance liquid chromatography over Column: Phenomenex Gemini-NX 80*40 mm*3 um to give the title compound: (RS)-2-(1-(2-hydroxyethyl)-1H-pyrazol-4-yl)-N-(2-methyl-5-(2-(2-methylpiperidin-1-yl)acetamido)pyridin-3-yl)pyrazolo[5,1-b]thiazole-7-carboxamide as a white solid (60 mg, 43%) as a yellow solid. LCMS(ESI): mass calcd for C₂₅H₃₀N₈O₃S, 522.62, m/z found, 523.1 [M+H]+. ¹H NMR (400 MHz, DMSO-d6) δ 9.91 (s, 1H), 9.84 (s, 1H), 8.58-8.62 (m, 2H), 8.53 (s, 1H), 8.21 (s, 1H), 8.16 (d, J=2.15 Hz, 1H), 7.90 (s, 1H), 4.97 (t, J=5.30 Hz, 1H), 4.18 (t, J=5.48 Hz, 2H), 3.77 (q, J=5.44 Hz, 2H), 3.08 (d, J=16.21 Hz, 1H), 2.82 (br d, J=11.21 Hz, 1H), 2.41 (s, 3H), 2.33-2.39 (m, 1H), 1.53-1.68 (m, 4H), 1.20-1.37 (m, 4H), 1.04 (d, J=6.32 Hz, 3H).

### Step c: (R)-2-(1-(2-hydroxyethyl)-1H-pyrazol-4-yl)-N-(2-methyl-5-(2-(2-methylpiperidin-1-yl)acetamido)pyridin-3-yl)pyrazolo[5,1-b]thiazole-7-carboxamide

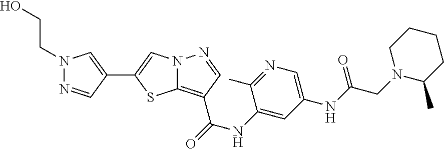 [SMILES: Cc1ncc(NC(=O)CN2CCCC[C@H]2C)cc1NC(=O)c1cnn2cc(-c3cnn(CCO)c3)sc12]

The residue was separated by Supercritical Fluid Chromatography over Column: DAICEL CHIRALCEL OD(250 mm*30 mm, 10 um) to give the title compound: (R)-2-(1-(2-hydroxyethyl)-1H-pyrazol-4-yl)-N-(2-methyl-5-(2-(2-methylpiperidin-1-yl)acetamido)pyridin-3-yl)pyrazolo[5,1-b]thiazole-7-carboxamide (27 mg, 44%). LCMS(ESI): mass calcd for C₂₅H₃₀N₈O₃S, 522.62, m/z found, 523.1 [M+H]+. ¹H NMR (400 MHz, DMSO-d6) δ 9.91 (s, 1H), 9.84 (s, 1H), 8.58-8.62 (m, 2H), 8.53 (s, 1H), 8.21 (s, 1H), 8.16 (d, J=2.15 Hz, 1H), 7.90 (s, 1H), 4.97 (t, J=5.30 Hz, 1H), 4.18 (t, J=5.48 Hz, 2H), 3.77 (q, J=5.44 Hz, 2H), 3.08 (d, J=16.21 Hz, 1H), 2.82 (br d, J=11.21 Hz, 1H), 2.41 (s, 3H), 2.33-2.39 (m, 1H), 1.53-1.68 (m, 4H), 1.20-1.37 (m, 4H), 1.04 (d, J=6.32 Hz, 3H).

### Example 34. (S)-2-(1-(2-hydroxyethyl)-1H-pyrazol-4-yl)-N-(2-methyl-5-(2-(2-methyl-piperidin-1-yl)acetamido)pyridin-3-yl)pyrazolo[5,1-b]thiazole-7-carboxamide

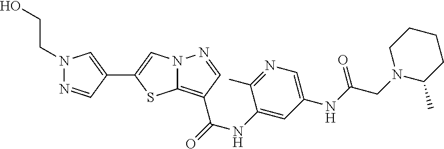 [SMILES: Cc1ncc(NC(=O)CN2CCCC[C@@H]2C)cc1NC(=O)c1cnn2cc(-c3cnn(CCO)c3)sc12]

### Step a: (S)-2-bromo-N-(2-methyl-5-(2-(2-methylpiperidin-1-yl)acetamido)pyridin-3-yl)pyrazolo[5,1-b]thiazole-7-carboxamide

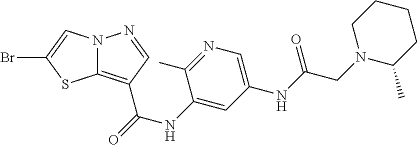 [SMILES: Cc1ncc(NC(=O)CN2CCCC[C@@H]2C)cc1NC(=O)c1cnn2cc(Br)sc12]

To a solution of 2-bromo-N-(5-(2-chloroacetamido)-2-methylpyridin-3-yl)pyrazolo[5,1-b]thiazole-7-carboxamide (120 mg, 0.28 mmol), (R)-2-methylpiperidine (33.3 mg, 0.34 mmol), and cesium carbonate (116.1 mg, 0.84 mmol) in DMF (3 mL) was added sodium iodide (25.1 mg, 0.17 mmol). The resulting mixture was stirred at 60° C. for 2 h. The mixture was concentrated under reduced pressure to give crude product, which was purified by column chromatography over silica gel (eluent: petroleum ether:ethyl acetate=4:1) to give the title compound: (S)-2-bromo-N-(2-methyl-5-(2-(2-methylpiperidin-1-yl)acetamido)pyridin-3-yl)pyrazolo[5,1-b]thiazole-7-carboxamide (100 mg, 74.8%). LCMS (ESI): mass calcd for C₂₀H₂₃BrN₆O₂S, 491.4; m/z found, 491.1 [M+H]+.

### Step b: (S)-2-(1-(2-hydroxyethyl)-1H-pyrazol-4-yl)-N-(2-methyl-5-(2-(2-methylpiperidin-1-yl)acetamido)pyridin-3-yl)pyrazolo[5,1-b]thiazole-7-carboxamide

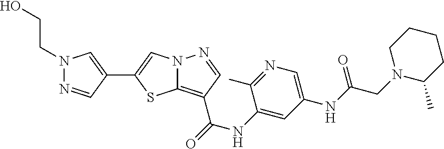 [SMILES: Cc1ncc(NC(=O)CN2CCCC[C@@H]2C)cc1NC(=O)c1cnn2cc(-c3cnn(CCO)c3)sc12]

To a solution of (S)-2-bromo-N-(2-methyl-5-(2-(2-methylpiperidin-1-yl)acetamido)pyridin-3-yl)pyrazolo[5,1-b]thiazole-7-carboxamide (100 mg, 0.2 mmol), 2-(4-(4,4,5,5-tetramethyl-1,3,2-dioxaborolan-2-yl)-1H-pyrazol-1-yl)ethanol (57.2 mg, 0.24 mmol), and potassium phosphate (127.5 mg, 0.6 mmol) in dioxane/H₂O=4:1 (5 mL) was added 1,1-bis(diphenylphosphino)ferrocene-palladium(II)dichloride dichloromethane complex (32.7 mg, 0.04 mmol) and the system was degassed by applying alternating N₂ atmosphere. The mixture was stirred at 100° C. for overnight, and then the reaction mixture was filtered and the filtrate was purified by preparative high-performance liquid chromatography over Column: Phenomenex Gemini-NX 80*40 mm*3 um to give the title compound: (S)-2-(1-(2-hydroxyethyl)-1H-pyrazol-4-yl)-N-(2-methyl-5-(2-(2-methylpiperidin-1-yl)acetamido)pyridin-3-yl)pyrazolo[5,1-b]thiazole-7-carboxamide as a white solid (31.2 mg, 29%) as a yellow solid. LCMS(ESI): mass calcd for C₂₅H₃₀N₈O₃S, 522.62, m/z found, 523.5 [M+H]+. ¹H NMR (400 MHz, METHANOL-d4) δ 8.58 (d, J=2.2 Hz, 1H), 8.38 (s, 1H), 8.24-8.21 (m, 2H), 8.07 (s, 1H), 7.84 (s, 1H), 4.26 (t, J=5.2 Hz, 2H), 3.90 (t, J=5.3 Hz, 2H), 3.51 (br d, J=15.2 Hz, 1H), 3.16 (br d, J=16.1 Hz, 1H), 2.95 (br s, 1H), 2.48 (s, 5H), 1.75-1.62 (m, 4H), 1.46-1.33 (m, 2H), 1.12 (d, J=6.2 Hz, 3H).

### Example 35. N-(5-((2-(5-azaspiro[3.4]octan-5-yl)ethyl)carbamoyl)-3-methylthiophen-2-yl)-2-(1-methyl-1H-pyrazol-4-yl)pyrazolo[5,1-b]thiazole-7-carboxamide

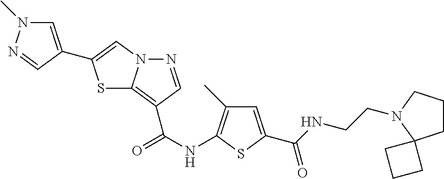 [SMILES: Cc1cc(C(=O)NCCN2CCCC23CCC3)sc1NC(=O)c1cnn2cc(-c3cnn(C)c3)sc12]

### Step a: methyl 5-(2-bromopyrazolo[5,1-b]thiazole-7-carboxamido)-4-methylthiophene-2-carboxylate

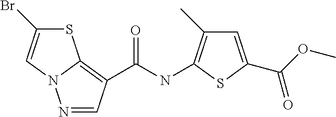 [SMILES: COC(=O)c1cc(C)c(NC(=O)c2cnn3cc(Br)sc23)s1]

A mixture of 2-bromopyrazolo[5,1-b]thiazole-7-carboxylic acid (4 g, 15.7 mmol) in sulfurous dichloride (10 ml) was stirred at 70° C. for 2 h. The reaction mixture was concentrated under vacuum to give the crude product as yellow solid 2-bromopyrazolo[5,1-b]thiazole-7-carbonyl chloride. 2-bromopyrazolo[5,1-b]thiazole-7-carbonyl chloride (3.5 g, 10.9 mmol) was added to a solution consisting of methyl 5-amino-4-methylthiophene-2-carboxylate (1.5 g, 8.75 mmol), TEA (4.57 ml, 32.8 mmol) and THF (350 mL) at room-temperature, The resulting mixture was stirred at 70° C. for 12 h. The resulting mixture was concentrated to the crude product, which was washed with dichloromethane (10*3 mL). The filtration was concentrated under reduced pressure to give the product methyl 5-(2-bromopyrazolo[5,1-b]thiazole-7-carboxamido)-4-methylthiophene-2-carboxylate (4.6 g, 95%) as a yellow solid. LCMS (ESI): mass calcd. for C₁₃H₁₀BrN₃O₃S₂, 400.2; m/z found, 401.9 [M+H]+.

### Step b: methyl 4-methyl-5-(2-(1-methyl-1H-pyrazol-4-yl)pyrazolo[5,1-b]thiazole-7-carboxamido)thiophene-2-carboxylate

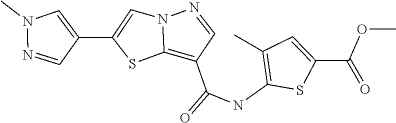

To a solution of methyl 5-(2-bromopyrazolo[5,1-b]thiazole-7-carboxamido)-4-methylthiophene-2-carboxylate (250 mg, 0.49 mmol) and 1-methyl-4-(4,4,5,5-tetramethyl-1,3,2-dioxaborolan-2-yl)-1H-pyrazole (49 mg, 0.24 mmol) in 1,4-dioxane (3 mL) and H₂O (0.75 mL) was added [1,1-bis(diphenylphosphino)ferrocene]palladium(II) chloride dichloromethane complex (74 mg, 0.09 mmol) and K₂CO₃ (0.37 g, 2.71 mmol) under N₂. The resulting mixture was stirred at 100° C. for 12 h before cooled to 25° C. The reaction mixture was diluted with ethyl acetate (10 mL) and filtered over celite. The filtration was concentrated to the crude product, which was treated with dichloromethane (10 mL) and water (10 mL). The organic phase was concentrated under reduced pressure to give the crude product, which was purified by column chromatography over silica gel (eluent: petroleum ether:ethyl acetate=0:1) to give the title compound methyl 4-methyl-5-(2-(1-methyl-1H-pyrazol-4-yl)pyrazolo[5,1-b]thiazole-7-carboxamido)thiophene-2-carboxylate (120 mg, 33%) as a yellow solid. LCMS (ESI): mass calcd. for C₁₇H₁₅N₅O₃S₂, 401.4; m/z found, 402.1 [M+H]⁺.

### Step c: 4-methyl-5-(2-(1-methyl-1H-pyrazol-4-yl)pyrazolo[5,1-b]thiazole-7-carboxamido)thiophene-2-carboxylic acid

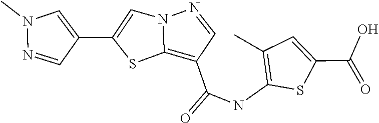

To a solution of methyl 4-methyl-5-(2-(1-methyl-1H-pyrazol-4-yl)pyrazolo[5,1-b]thiazole-7-carbox-amido)thiophene-2-carboxylate (120 mg, 0.3 mmol) in ethanol (1 mL) was added sodium hydroxide (0.5 ml, 2 M in water, 1 mmol) at room temperature. The reaction mixture was stirred at 50° C. for 2 h before cooling to room-temperature. The mixture was adjusted to pH=3-4 with HCl (aq, 2 M). The mixture was filtered and washed with water (10 mL×3). The solid was evaporated under vacuum to give desired product 4-methyl-5-(2-(1-methyl-1H-pyrazol-4-yl)pyrazolo[5,1-b]thiazole-7-carboxamido)thiophene-2-carboxylic acid (90 mg, 78%) as white solid. LCMS (ESI): mass calcd. for C₁₆H₁₃N5O₃S₂, 387.4; m/z found, 388.0 [M+H]+.

### Step d: N-(5-((2-(5-azaspiro[3.4]octan-5-yl)ethyl)carbamoyl)-3-methylthiophen-2-yl)-2-(1-methyl-1H-pyrazol-4-yl)pyrazolo[5,1-b]thiazole-7-carboxamide

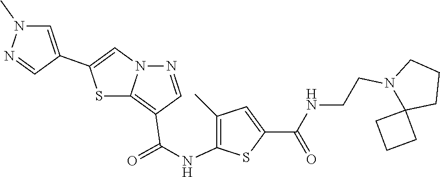 [SMILES: Cc1cc(C(=O)NCCN2CCCC23CCC3)sc1NC(=O)c1cnn2cc(-c3cnn(C)c3)sc12]

To a solution of 4-methyl-5-(2-(1-methyl-1H-pyrazol-4-yl)pyrazolo[5,1-b]thiazole-7-carboxamido)thiophene-2-carboxylic acid (80 mg, 0.21 mmol), 2-(5-azaspiro[3.4]octan-5-yl)ethanamine (35 mg, 0.23 mmol) and N,N-diisopropylethylamine (80 mg, 0.62 mmol) in DMF (4 mL) was added HATU (92 mg, 0.25 mmol). The resulting mixture was stirred at 30° C. for 2 hours and then concentrated under vacuum to give the crude product, which was purified by preparative high-performance liquid chromatography over Column: Boston Green ODS 150*30 mm*5 um to give the title compound N-(5-((2-(5-azaspiro[3.4]octan-5-yl)ethyl)carbamoyl)-3-methylthiophen-2-yl)-2-(1-methyl-1H-pyrazol-4-yl)pyrazolo[5,1-b]thiazole-7-carboxamide (30 mg, 26%) as a white solid. LCMS (ESI): mass calcd. for C₂₅H₂₉N₇O₂S₂, 523.6; m/z found, 524.2 [M+H]⁺. ¹H NMR (400 MHz, METHANOL-d4) δ 8.45 (s, 1H), 8.21 (s, 1H), 8.03 (s, 1H), 7.82 (s, 1H), 7.41 (s, 1H), 3.94 (s, 3H), 3.68 (t, J=6.3 Hz, 2H), 3.36 (br t, J=7.5 Hz, 2H), 3.22 (br t, J=6.0 Hz, 2H), 2.55-2.45 (m, 2H), 2.30 (s, 3H), 2.25-2.20 (m, 2H), 2.09-1.86 (m, 6H).

### Example 36. N-(5-((2-(2,2-dimethylpyrrolidin-1-yl)ethyl)carbamoyl)-3-methylthiophen-2-yl)-2-(pyridin-3-yl)pyrazolo[5,1-b]thiazole-7-carboxamide

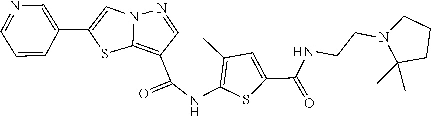 [SMILES: Cc1cc(C(=O)NCCN2CCCC2(C)C)sc1NC(=O)c1cnn2cc(-c3cccnc3)sc12]

### Step a: 2-bromo-N-(5-((2-(2,2-dimethylpyrrolidin-1-yl)ethyl)carbamoyl)-3-methylthiophen-2-yl)pyrazolo[5,1-b]thiazole-7-carboxamide

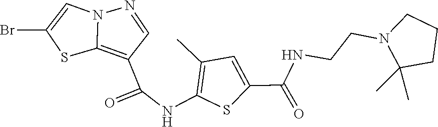 [SMILES: Cc1cc(C(=O)NCCN2CCCC2(C)C)sc1NC(=O)c1cnn2cc(Br)sc12]

To a solution of 5-(2-bromopyrazolo[5,1-b]thiazole-7-carboxamido)-4-methylthiophene-2-carboxylic acid (2.1 g, 5.44 mmol), 2-(2,2-dimethylpyrrolidin-1-yl)ethanamine (0.85 g, 5.98 mmol) and N,N-diisopropylethylamine (2.11 g, 16.3 mmol) in DMF (25 mL) was added HATU (2.48 g, 6.52 mmol). The resulting mixture was stirred at 30° C. for 12 hours and then concentrated under vacuum to give the crude product, which was purified by column chromatography over silica gel (eluent: (dichloromethane:methanol=7:3) to give the title compound 2-bromo-N-(5-((2-(2,2-dimethylpyrrolidin-1-yl)ethyl)carbamoyl)-3-methylthiophen-2-yl)pyrazolo[5,1-b]thiazole-7-carboxamide (600 mg, 22%) as a pale yellow solid. LCMS (ESI): mass calcd. for C₂₀H₂₄BrN₅O₂S₂, 510.5; m/z found, 510.0 [M+H]+

### Step b: N-(5-((2-(2,2-dimethylpyrrolidin-1-yl)ethyl)carbamoyl)-3-methylthiophen-2-yl)-2-(pyridin-3-yl)pyrazolo[5,1-b]thiazole-7-carboxamide

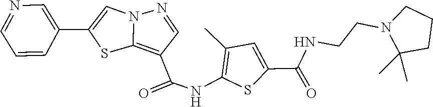 [SMILES: Cc1cc(C(=O)NCCN2CCCC2(C)C)sc1NC(=O)c1cnn2cc(-c3cccnc3)sc12]

To a solution of methyl 2-bromo-N-(5-((2-(2,2-dimethylpyrrolidin-1-yl)ethyl)carbamoyl)-3-methylthiophen-2-yl)pyrazolo[5,1-b]thiazole-7-carboxamide (120 mg, 0.24 mmol) and pyridin-3-ylboronic acid (43 mg, 0.35 mmol) in 1,4-dioxane (4 mL) and H₂O (1 mL) was added [1,1-bis(diphenylphosphino)ferrocene]palladium(II) chloride dichloromethane complex (19 mg, 0.02 mmol) and K₃PO₄ (150 mg, 0.71 mmol) under N₂. The resulting mixture was stirred at 95° C. for 12 h before cooled to 25° C. The reaction mixture was diluted with ethyl acetate (10 mL) and filtered over celite. The filtration was concentrated to the crude product, which was purified by preparative high-performance liquid chromatography over Column: Boston Green ODS 150*30 mm*5 um to give the title compound N-(5-((2-(2,2-dimethylpyrrolidin-1-yl)ethyl)carbamoyl)-3-methylthiophen-2-yl)-2-(pyridin-3-yl)pyrazolo[5,1-b]thiazole-7-carboxamide (18 mg, 15%) as a yellow solid. LCMS (ESI): mass calcd. for C₂₅H₂₈N₆O₂S₂, 508.6; m/z found, 509.1 [M+H]⁺. ¹H NMR (400 MHz, METHANOL-d4) δ 8.93 (d, J=2.0 Hz, 1H), 8.69 (s, 1H), 8.59 (d, J=4.0 Hz, 1H), 8.54 (s, 1H), 8.17 (br d, J=8.0 Hz, 1H), 7.57 (dd, J=4.9, 7.9 Hz, 1H), 7.42 (s, 1H), 3.65 (br t, J=6.3 Hz, 2H), 3.53-3.33 (m, 2H), 3.15 (br s, 2H), 2.31 (s, 3H), 2.12-2.03 (m, 2H), 2.01-1.94 (m, 2H), 1.32 (s, 6H)

### Example 37. N-(5-((2-(4-azaspiro[2.4]heptan-4-yl)ethyl)carbamoyl)-3-methylthiophen-2-yl)-2-(1-methyl-1H-pyrazol-4-yl)pyrazolo[5,1-b]thiazole-7-carboxamide

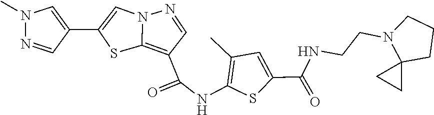 [SMILES: Cc1cc(C(=O)NCCN2CCCC23CC3)sc1NC(=O)c1cnn2cc(-c3cnn(C)c3)sc12]

### Step a: methyl 4-methyl-5-(2-(1-methyl-1H-pyrazol-4-yl)pyrazolo[5,1-b]thiazole-7-carboxamido)thiophene-2-carboxylate

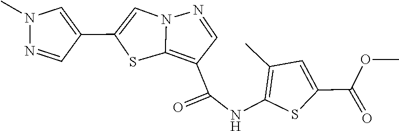

To a solution of methyl 5-(2-bromopyrazolo[5,1-b]thiazole-7-carboxamido)-4-methylthiophene-2-carboxylate (250 mg, 0.495 mmol) and 1-methyl-4-(4,4,5,5-tetramethyl-1,3,2-dioxaborolan-2-yl)-1H-pyrazole (49 mg, 0.24 mmol) in 1,4-dioxane (3 mL) and H₂O (0.75 mL) was added [1,1-Bis(diphenylphosphino)ferrocene]palladium(II) chloride dichloromethane complex (74 mg, 0.09 mmol) and K₂CO₃ (0.37 g, 2.71 mmol) under N₂. The resulting mixture was stirred at 100° C. for 12 h before cooled to 25° C. The reaction mixture was diluted with ethyl acetate (10 mL) and filtered over celite. The filtration was concentrated to the crude product, which was treated with dichloromethane (10 mL) and water (10 mL). The organic phase was concentrated under reduced pressure to give the crude product, which was purified by column chromatography over silica gel (eluent: petroleum ether:ethyl acetate=0:1) to give the title compound methyl 4-methyl-5-(2-(1-methyl-1H-pyrazol-4-yl)pyrazolo[5,1-b]thiazole-7-carboxamido)thiophene-2-carboxylate (120 mg, 33%) as a yellow solid. LCMS (ESI): mass calcd. for C₁₇H₁₅N₅O₃S₂, 401.4; m/z found, 402.1 [M+H]⁺.

### Step b: 4-methyl-5-(2-(1-methyl-1H-pyrazol-4-yl)pyrazolo[5,1-b]thiazole-7-carboxamido)thiophene-2-carboxylic acid

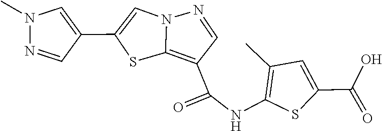 [SMILES: Cc1cc(C(=O)O)sc1NC(=O)c1cnn2cc(-c3cnn(C)c3)sc12]

To a solution of methyl 4-methyl-5-(2-(1-methyl-1H-pyrazol-4-yl)pyrazolo[5,1-b]thiazole-7-carbox-amido)thiophene-2-carboxylate (120 mg, 0.30 mmol) in ethanol (1 mL) was added sodium hydroxide (0.5 ml, 2 M in water, 1 mmol) at room temperature. The reaction mixture was stirred at 50° C. for 2 h before cooling to room-temperature. The mixture was adjusted to pH=3-4 with HCl (aq, 2 M). The mixture was filtered and washed with water (10 mL×3). The solid was evaporated under vacuum to give desired product 4-methyl-5-(2-(1-methyl-1H-pyrazol-4-yl)pyrazolo[5,1-b]thiazole-7-carboxamido)thiophene-2-carboxylic acid (90 mg, 78%) as white solid. LCMS (ESI): mass calcd. for C_{1M}H₁₃N₅O₃S₂, 387.4; m/z found, 388.0 [M+H]+.

### Step c: N-(5-((2-(4-azaspiro[2.4]heptan-4-yl)ethyl)carbamoyl)-3-methylthiophen-2-yl)-2-(1-methyl-1H-pyrazol-4-yl)pyrazolo[5,1-b]thiazole-7-carboxamide

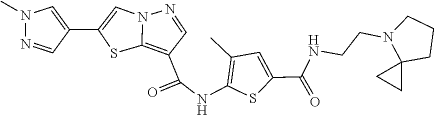 [SMILES: Cc1cc(C(=O)NCCN2CCCC23CC3)sc1NC(=O)c1cnn2cc(-c3cnn(C)c3)sc12]

To a solution of 4-methyl-5-(2-(1-methyl-1H-pyrazol-4-yl)pyrazolo[5,1-b]thiazole-7-carboxamido)thiophene-2-carboxylic acid (80 mg, 0.21 mmol), 2-(4-azaspiro[2.4]heptan-4-yl)ethan-1-amine (32 mg, 0.23 mmol) and N,N-diisopropylethylamine (80 mg, 0.62 mmol) in DMF (3 mL) was added HATU (92 mg, 0.25 mmol). The resulting mixture was stirred at 30° C. for 12 hours and then concentrated under vacuum to give the crude product, which was purified by preparative high-performance liquid chromatography over Column: Boston Green ODS 150*30 mm*5 um to give the title compound N-(5-((2-(4-azaspiro[2.4]heptan-4-yl)ethyl)carbamoyl)-3-methylthiophen-2-yl)-2-(1-methyl-1H-pyrazol-4-yl)pyrazolo[5,1-b]thiazole-7-carboxamide (22 mg, 20%) as a yellow solid. LCMS (ESI): mass calcd. for C₂₄H₂₇N₇O₂S₂, 509.6; m/z found, 510.2 [M+H]⁺. ¹H NMR (400 MHz, METHANOL-d4) δ 8.46 (s, 1H), 8.23 (s, 1H), 8.04 (s, 1H), 7.83 (s, 1H), 7.40 (s, 1H), 3.95 (s, 3H), 3.61 (t, J=6.3 Hz, 2H), 3.52 (br t, J=7.0 Hz, 2H), 3.02 (t, J=6.1 Hz, 2H), 2.30 (s, 3H), 2.23-2.14 (m, 2H), 2.12-2.05 (m, 2H), 1.23-1.17 (m, 2H), 0.88-0.81 (m, 2H).

### Example 38. N-(5-(3-(2-(2,2-dimethylpyrrolidin-1-yl)ethyl)ureido)-2-methyl-pyridin-3-yl)-2-(1-methyl-1H-pyrazol-4-yl)pyrazolo[5,1-b]thiazole-7-carboxamide

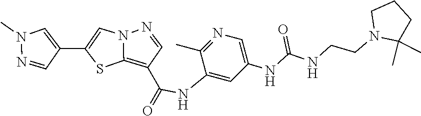

### Step a: phenyl (2-(2,2-dimethylpyrrolidin-1-yl)ethyl)carbamate

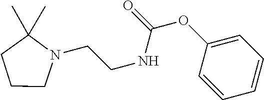 [SMILES: CC1(C)CCCN1CCNC(=O)Oc1ccccc1]

To a solution of 2-(2,2-dimethylpyrrolidin-1-yl)ethanamine (3 g, 21 mmol) in dichloromethane (30 ml) was added phenyl chloroformate (2.65 mL, 21 mmol). The mixture was stirred at 0° C. for 1.5 hours. The reaction mixture was concentrated under reduced pressure to give crude product, which was used to next step without further purification to give the title compound (6 g, 47%) as a red oil. LCMS (ESI): mass calcd. for C₁₅H₂₂N₂O₂, 262.35; m/z found, 263.0 [M+H]⁺.

### Step b: 1-(2-(2,2-dimethylpyrrolidin-1-yl)ethyl)-3-(6-methyl-5-nitropyridin-3-yl)urea

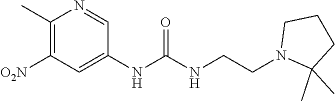 [SMILES: Cc1ncc(NC(=O)NCCN2CCCC2(C)C)cc1[N+](=O)[O-]]

To a solution of phenyl (2-(2,2-dimethylpyrrolidin-1-yl)ethyl)carbamate (1.67 g, 2.79 mmol), 6-methyl-5-nitropyridin-3-amine (214 mg, 1.40 mmol) in acetonitrile (10 ml) was added 4-dimethylaminopyridine (340 mg, 2.80 mmol). The mixture was stirred at 80° C. for 12 hours. The reaction mixture was concentrated under reduced pressure to give crude product, which was purified by preparative high-performance liquid chromatography over Column: Boston Uni C18 40*150*5 um) to give the title compound (300 mg, 33%) as a red brown oil. LCMS (ESI): mass calcd. for C₁₅H₂₃N₅O₃, 321.37; m/z found, 322.0 [M+H]⁺.

### Step c: 1-(5-amino-6-methylpyridin-3-yl)-3-(2-(2,2-dimethylpyrrolidin-1-yl)ethyl)urea

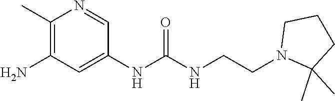 [SMILES: Cc1ncc(NC(=O)NCCN2CCCC2(C)C)cc1N]

To a solution of 1-(2-(2,2-dimethylpyrrolidin-1-yl)ethyl)-3-(6-methyl-5-nitropyridin-3-yl)urea (1.6 g, 4.98 mmol) in methanol (10 mL) was hydrogenated at 25° C. (15 psi) with Pd/C (212 mg, 10%) as a catalyst in the presence of H₂ for 12 hours. After uptake of H₂ (3 equivalent), the catalyst filtered off and the filtrate was evaporated give the crude product as yellow oil. The residue was purified by preparative high-performance liquid chromatography over Column: Xtimate C18 150*40 mm*5 um to give the title compound (440 mg, 30%) as a white solid. LCMS (ESI): mass calcd. for C₁₅H₂₅N50, 291.39; m/z found, 292.3 [M+H]⁺. ¹H NMR (400 MHz, METHANOL-d4)) δ 7.67 (d, J=2.2 Hz, 1H), 7.24 (d, J=2.2 Hz, 1H), 3.27-3.23 (m, 2H), 2.82 (br t, J=7.2 Hz, 2H), 2.56 (br t, J=6.4 Hz, 2H), 2.25 (s, 3H), 1.85-1.74 (m, 2H), 1.70-1.62 (m, 2H), 1.01 (s, 6H).

### Step d: 2-bromo-N-(5-(3-(2-(2,2-dimethylpyrrolidin-1-yl)ethyl)ureido)-2-methylpyridin-3-yl) pyrazolo[5,1-b]thiazole-7-carboxamide

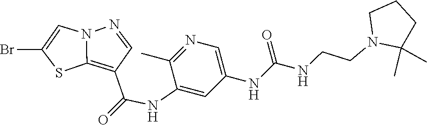 [SMILES: Cc1ncc(NC(=O)NCCN2CCCC2(C)C)cc1NC(=O)c1cnn2cc(Br)sc12]

To a solution of 2-bromopyrazolo[5,1-b]thiazole-7-carboxylic acid (397 mg, 1.6 mmol) in thionyl chloride (10 ml, 137.5 mmol) was stirred at 90° C. for 2 hours. The reaction mixture was concentrated under vacuum to give the crude product as white solid 2-bromopyrazolo[5,1-b]thiazole-7-carbonyl chloride. 2-bromopyrazolo[5,1-b]thiazole-7-carbonyl chloride (397 mg, 1.50 mmol) was added to a solution consisting of 1-(5-amino-6-methylpyridin-3-yl)-3-(2-(2,2-dimethylpyrrolidin-1-yl)ethyl)urea (390 mg, 1.34 mmol) in pyridine (10 mL) at 25° C. for 12 hours. The reaction mixture was concentrated under reduced pressure to give crude product, which was purified by preparative high-performance liquid chromatography over Column: Phenomenex Gemini-NX C18 75*30 mm*3 um to give the title compound (560 mg, 80%) as a yellow solid. LCMS (ESI): mass calcd. for C₂₁H₂₆BrN₇O₂S, 520.4; m/z found, 522.0 [M+H]⁺. ¹H NMR (400 MHz, METHANOL-d4) δ 8.47 (s, 1H), 8.43 (d, J=2.3 Hz, 1H), 8.36 (s, 1H), 8.10 (d, J=2.3 Hz, 1H), 3.59 (br t, J=5.7 Hz, 4H), 3.26 (br s, 2H), 2.48 (s, 3H), 2.20-2.10 (m, 2H), 2.09-2.01 (m, 2H), 1.51-1.32 (m, 6H).

### Step e: N-(5-(3-(2-(2,2-dimethylpyrrolidin-1-yl)ethyl)ureido)-2-methylpyridin-3-yl)-2-(1-methyl-1H-pyrazol-4-yl)pyrazolo[5,1-b]thiazole-7-carboxamide

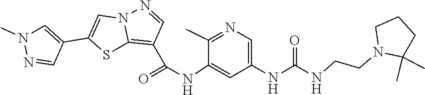

To a solution of 2-bromo-N-(5-(3-(2-(2,2-dimethylpyrrolidin-1-yl)ethyl)ureido)-2-methylpyridin-3-yl)pyrazolo[5,1-b]thiazole-7-carboxamide (300 mg, 0.58 mmol) and 1-methyl-4-(4,4,5,5-tetramethyl-1,3,2-dioxaborolan-2-yl)-1H-pyrazole (180 mg, 0.87 mmol) in 1,4-dioxane and water (30 mL, 4:1) was added [1,1-bis (diphenylphosphino)ferrocene]palladium(II) chloride dichloromethane complex (94 mg, 0.12 mmol) and cesium carbonate (563 mg, 1.73 mmol) under N₂, the yellow mixture stirred at 100° C. for 8 hours. The mixture was cooled to 25° C., and then diluted with ethyl acetate (30 mL), filtered and the filtrate was concentrated to remove solvent to give a residue. The crude product was purified by preparative high-performance liquid chromatography over Column: Phenomenex Gemini-NX 80*40 mm*3 um to give the title compound (53 mg, 17%) as a white solid. LCMS (ESI): mass calcd. for C₂₅H₃₁N₉O₂S, 506.6; m/z found, 507.1 [M+H]⁺. ¹H NMR (400 MHz, METHANOL-d4) δ 8.41-8.31 (m, 2H), 8.19 (s, 1H), 8.02 (br d, J=2.2 Hz, 1H), 8.00 (s, 1H), 7.80 (s, 1H), 3.92 (s, 3H), 3.35-3.30 (m, 2H), 2.88 (br s, 2H), 2.62 (br s, 2H), 2.43 (s, 3H), 1.88-1.75 (m, 2H), 1.74-1.62 (m, 2H), 1.04 (s, 6H).

### Example 39. 2-(6,7-dihydro-5H-pyrazolo[5,1-b][1,3]oxazin-3-yl)-N-(5-(2-(3,3-dimethylazetidin-1-yl)acetamido)-2-methylpyridin-3-yl)pyrazolo[5,1-b]thiazole-7-carboxamide

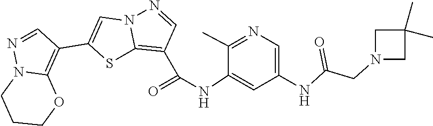 [SMILES: Cc1ncc(NC(=O)CN2CC(C)(C)C2)cc1NC(=O)c1cnn2cc(-c3cnn4c3OCCC4)sc12]

### Step a: 2-bromo-N-(5-(2-(3,3-dimethylazetidin-1-yl)acetamido)-2-methylpyridin-3-yl)pyrazolo[5,1-b]thiazole-7-carboxamide

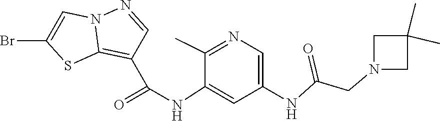 [SMILES: Cc1ncc(NC(=O)CN2CC(C)(C)C2)cc1NC(=O)c1cnn2cc(Br)sc12]

To a mixture of 2-bromo-N-(5-(2-chloroacetamido)-2-methylpyridin-3-yl)pyrazolo[5,1-b]thiazole-7-carboxamide (1.29 g, 3.01 mmol), 3,3-dimethylazetidine hydrochloride (409 mg, 3.36 mmol), and cesium carbonate (2.16 g, 6.62 mmol) under nitrogen was added DMF (10 mL) and the reaction was heated at 50° C. for 12 h. The reaction was filtered and purified by prep-HPLC (25%-45% MeCN/water/10 mM NH₄OH) to yield 2-bromo-N-(5-(2-(3,3-dimethylazetidin-1-yl)acetamido)-2-methylpyridin-3-yl)pyrazolo[5,1-b]thiazole-7-carboxamide (0.9 g, 61%) as a white solid. LCMS (ESI): mass calcd. for C₁₅H₂₂N₂O₂, 477.38; m/z found, 477.0/479.0 [M+H]⁺.

### Step b: 2-(6,7-dihydro-5H-pyrazolo[5,1-b][1,3]oxazin-3-yl)-N-(5-(2-(3,3-dimethylazetidin-1-yl)acetamido)-2-methylpyridin-3-yl)pyrazolo[5,1-b]thiazole-7-carboxamide

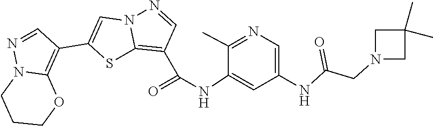 [SMILES: Cc1ncc(NC(=O)CN2CC(C)(C)C2)cc1NC(=O)c1cnn2cc(-c3cnn4c3OCCC4)sc12]

A mixture of 2-bromo-N-(5-(2-(3,3-dimethylazetidin-1-yl)acetamido)-2-methylpyridin-3-yl)pyrazolo[5,1-b]thiazole-7-carboxamide (47 mg, 0.096 mmol), 3-(4,4,5,5-tetramethyl-1,3,2-dioxaborolan-2-yl)-6,7-dihydro-5H-pyrazolo[5,1-B][1,3]oxazine (48 mg, 0.19 mmol), Cs₂CO₃ (112 mg, 0.34 mmol), and PdCl₂(dppf)·CH₂Cl₂ (20 mg, 0.025 mmol) in 1,4-dioxane (1.4 mL), water (0.4 mL), and (0.5 mL) under nitrogen in a capped 5 mL microwave vial was sparged with nitrogen for 10 minutes, then heated at 130° C. for 1 h. The reaction was cooled to rt, stirred with Si-trisamine for 20 min, filtered, and purified by prep-HPLC, 5%-32% MeCN/water/0.1% TFA to yield 2-(6,7-dihydro-5H-pyrazolo[5,1-b][1,3]oxazin-3-yl)-N-(5-(2-(3,3-dimethylazetidin-1-yl)acetamido)-2-methylpyridin-3-yl)pyrazolo[5,1-b]thiazole-7-carboxamide (28 mg, 46%) as a pale yellow solid. LCMS (ESI): mass calcd. for C₂₅H₂₈N₈O₃S, 520.6; m/z found, 521.2 [M+H]⁺. ¹H NMR (METHANOL-d4) δ 8.67 (d, J=1.5 Hz, 1H), 8.33-8.39 (m, 2H), 8.09 (s, 1H), 7.68 (s, 1H), 4.49-4.56 (m, 2H), 4.31 (s, 2H), 4.20 (t, J=6.1 Hz, 2H), 3.94-4.16 (m, 4H), 2.57 (s, 3H), 2.31-2.39 (m, 2H), 1.31-1.50 (m, 6H).

### Example 40. N-(5-(2-(3,3-dimethylazetidin-1-yl)acetamido)-2-methylpyridin-3-yl)-2-(4-methoxypyridin-3-yl)pyrazolo[5,1-b]thiazole-7-carboxamide

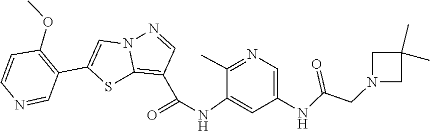 [SMILES: COc1ccncc1-c1cn2ncc(C(=O)Nc3cc(NC(=O)CN4CC(C)(C)C4)cnc3C)c2s1]

A mixture of 2-bromo-N-(5-(2-(3,3-dimethylazetidin-1-yl)acetamido)-2-methylpyridin-3-yl)pyrazolo[5,1-b]thiazole-7-carboxamide (50 mg, 0.1 mmol), 4-methoxypyridine-3-boronic acid hydrate (35 mg, 0.21 mmol), Cs₂CO₃ (113 mg, 0.35 mmol), and PdCl₂(dppf)·CH₂Cl₂ (17 mg, 0.021 mmol) in 1,4-dioxane (1.8 mL) and water (0.5 mL) in a capped 5 mL microwave vial under nitrogen was sparged with nitrogen for 10 min, then heated at 130° C. in the microwave for 1.5 h. An addition equivalent of 4-methoxypyridine-3-boronic acid hydrate and 0.2 equivalent of PdCl₂(dppf)·CH₂Cl₂ were added, the vial was recapped, placed under vacuum, back-filled with nitrogen 3×, then heated at 130° in the microwave for 1 h. The reaction was cooled to rt, stirred with Si-trisamine for 30 min, filtered, and purified by prep-HPLC, 20%-40% MeCN/water/10 mM NH₄OH. The product fractions were concentrated to dryness on the rotovap, taken up in DMF (2 mL), and purified by prep-HPLC, 0%-30% MeCN/water/0.1% TFA to yield N-(5-(2-(3,3-dimethylazetidin-1-yl)acetamido)-2-methylpyridin-3-yl)-2-(4-methoxypyridin-3-yl)pyrazolo[5,1-b]thiazole-7-carboxamide (9 mg, 14%) as a white solid. LCMS (ESI): mass calcd. for C₂₅H₂₇N₇O₃S, 505.6; m/z found, 506.2 [M+H]⁺. ¹H NMR (METHANOL-d4) δ 8.99 (s, 1H), 8.81 (s, 1H), 8.67 (d, J=6.4 Hz, 1H), 8.64 (d, J=2.0 Hz, 1H), 8.51 (s, 1H), 8.33 (d, J=2.4 Hz, 1H), 7.65 (d, J=6.8 Hz, 1H), 4.31 (s, 2H), 4.28 (s, 3H), 3.93-4.16 (m, 4H), 2.56 (s, 3H), 1.32-1.50 (m, 6H).

### Example 41. N-(5-(3-(2,2-dimethylpyrrolidin-1-yl)propanamido)-2-methyl pyridin-3-yl)-2-(1-(2-methoxyethyl)-1H-pyrazol-4-yl)pyrazolo[5,1-b]thiazole-7-carboxamide

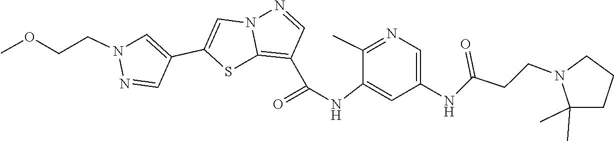 [SMILES: COCCn1cc(-c2cn3ncc(C(=O)Nc4cc(NC(=O)CCN5CCCC5(C)C)cnc4C)c3s2)cn1]

### Step a: tert-butyl (5-(2-(1-(2-methoxyethyl)-1H-pyrazol-4-yl)pyrazolo[5,1-b]thiazole-7-carboxamido)-6-methylpyridin-3-yl)carbamate

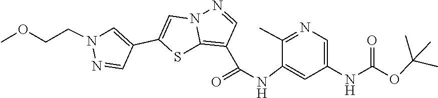 [SMILES: COCCn1cc(-c2cn3ncc(C(=O)Nc4cc(NC(=O)OC(C)(C)C)cnc4C)c3s2)cn1]

To a solution of tert-butyl (5-(2-bromopyrazolo[5,1-b]thiazole-7-carboxamido)-6-methylpyridin-3-yl)carbamate (12 g, 26.5 mmol), 1-(2-Methoxyethyl)-4-(4,4,5,5-Tetramethyl-1,3,2-Dioxaborolan-2-Yl)-1H-Pyrazole (8.4 g, 33.3 mmol) and Cs₂CO₃ (25.8 g, 79.2 mmol) in dioxane/H₂O (1500 mL, 4/1) was added PdCl₂(dppf)·CH₂Cl₂ (6.6 g, 8.1 mmol) at room-temperature under an atmosphere of nitrogen. The reaction vessel was gradually warmed to 100° C. over the course of 10 min, after which time stirring was continued for 12 hours. During this time, the reaction mixture went from a yellow coloration to red brown (rust-like) and then finally black. The reaction mixture was concentrated to dryness in vacuum give black solid. The black solid was treated with dichloromethane/methanol (50 mL, 5/1). The reaction mixture was filtered and the filter cake was rinsed with 50 mL of dichloromethane. The filtrate was collected, dried in vacuum to give a black solid. The black solid was subjected to column chromatography over silica gel (gradient elution: 0 ˜10% methanol in dichloromethane). The pure fractions were collected and concentrated to dryness in vacuum to give tert-butyl (5-(2-(1-(2-methoxyethyl)-1H-pyrazol-4-yl)pyrazolo[5,1-b]thiazole-7-carboxamido)-6-methylpyridin-3-yl)carbamate (7.5 g, 13.9 mmol, 53% yield) as a brown solid. LCMS (ESI): mass calcd. for C₂₃H₂₇N₇O₄S, 497.18; m/z found, 498[M+H]⁺.

### Step b: N-(5-amino-2-methylpyridin-3-yl)-2-(1-(2-methoxyethyl)-1H-pyrazol-4-yl)pyrazolo[5,1-b]thiazole-7-carboxamide

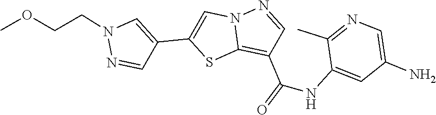 [SMILES: COCCn1cc(-c2cn3ncc(C(=O)Nc4cc(N)cnc4C)c3s2)cn1]

To the solution of tert-butyl (5-(2-(1-(2-methoxyethyl)-1H-pyrazol-4-yl)pyrazolo[5,1-b]thiazole-7-carboxamido)-6-methylpyridin-3-yl)carbamate (7.5 g, 13.9 mmol) in DCM (100 mL) and MeOH (20 mL) was added HCl/dioxane (80 mL, 320 mmol, 4 M). The resulting mixture was stirred at r.t. for 16 hours. The reaction mixture was concentrated to dryness to give N-(5-amino-2-methylpyridin-3-yl)-2-(1-(2-methoxyethyl)-1H-pyrazol-4-yl)pyrazolo[5,1-b]thiazole-7-carboxamide (6.6 g, 87%) as a brown solid (HCl salt). LCMS (ESI): mass calcd. for C₁₈H₁₉N₇O₂S, 397.5; m/z found, 398.2 [M+H]⁺.

### Step c: N-(5-(3-chloropropanamido)-2-methylpyridin-3-yl)-2-(1-(2-methoxyethyl)-1H-pyrazol-4-yl)pyrazolo[5,1-b]thiazole-7-carboxamide

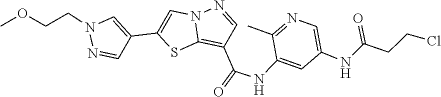 [SMILES: COCCn1cc(-c2cn3ncc(C(=O)Nc4cc(NC(=O)CCCl)cnc4C)c3s2)cn1]

To a solution of N-(5-amino-2-methylpyridin-3-yl)-2-(1-(2-methoxyethyl)-1H-pyrazol-4-yl)pyrazolo[5,1-b]thiazole-7-carboxamide (1.1 g, 2.0 mmol) in CHCl₃ (77 mL) and H₂O (77 mL) was added NaHCO₃ (10 g, 119 mmol) and 3-chloropropanoyl chloride (4 mL, 41.9 mmol) dropwise via syringe at room temperature over 5 min. The mixture was stirred at room temperature for 1 hour. The reaction mixture became cloudy. Color changes (brown to black) were observed. Water (1000 mL) was added to the mixture and the mixture was extracted with ethyl acetate (1000 mL×4). The organic layer was dried over anhydrous Na₂SO₄, filtered, and concentrated to dryness in vacuo to give N-(5-(3-chloropropanamido)-2-methylpyridin-3-yl)-2-(1-(2-methoxyethyl)-1H-pyrazol-4-yl)pyrazolo[5,1-b]thiazole-7-carboxamide (900 mg, 1.8 mmol, 88% yield) as a brown solid. LCMS (ESI): mass calcd. for C₂₁H₂₂ClN₇O₃S, 488.0; m/z found, 488.1 [M+H]⁺.

### Step d: N-(5-(3-(2,2-dimethylpyrrolidin-1-yl)propanamido)-2-methylpyridin-3-yl)-2-(1-(2-methoxyethyl)-1H-pyrazol-4-yl)pyrazolo[5,1-b]thiazole-7-carboxamide

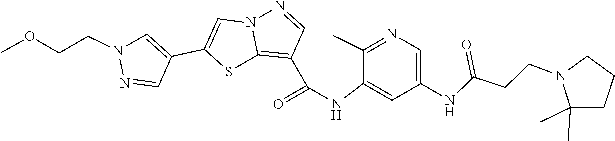 [SMILES: COCCn1cc(-c2cn3ncc(C(=O)Nc4cc(NC(=O)CCN5CCCC5(C)C)cnc4C)c3s2)cn1]

To a solution of N-(5-(3-chloropropanamido)-2-methylpyridin-3-yl)-2-(1-(2-methoxyethyl)-1H-pyrazol-4-yl)pyrazolo[5,1-b]thiazole-7-carboxamide (5.5 g, 10.8 mmol) in MeCN (60 mL) was added 2,2-dimethylpyrrolidine hydrochloride (6.1 g, 44.6 mmol), triethylamine (12.1 mL, 86.8 mmol) at r.t. The reaction mixture was stirred at 70° C. for 16 hours. The reaction mixture was become cloudy. The reaction mixture was cooled to r.t. concentrated to dryness in vacuo to give a black solid. The black solid was subjected to column chromatography over silica gel (gradient elution: 0˜ 38.5% methanol in dichloromethane). The pure fractions were collected and concentrated to dryness in vacuum to give N-(5-(3-(2,2-dimethylpyrrolidin-1-yl)propanamido)-2-methylpyridin-3-yl)-2-(1-(2-methoxyethyl)-1H-pyrazol-4-yl)pyrazolo[5,1-b]thiazole-7-carboxamide (2.6 g, 4.7 mmol, 43% yield) as a gray solid. LCMS (ESI): mass calcd. for C₂₇H₃₄N₈O₃S, 550.7; m/z found, 551.3 [M+H]⁺. ¹H NMR (400 MHz, CHLOROFORM-d) δ ppm 11.39 (br s, 1H), 8.46 (s, 1H), 8.30 (s, 1H), 8.06 (s, 1H), 7.72 (s, 1H), 7.59-7.70 (m, 3H), 4.26 (t, J=5.01 Hz, 2H), 3.71 (t, J=5.07 Hz, 2H), 3.30 (s, 3H), 2.90 (br t, J=6.20 Hz, 2H), 2.81 (br t, J=5.66 Hz, 2H), 2.56 (br t, J=5.72 Hz, 2H), 2.45 (s, 3H), 1.75-1.83 (m, 4H), 1.08 (s, 6H).

### Example 42. N-(5-(3-(2,2-dimethylpyrrolidin-1-yl)propanamido)-2-methyl-pyridin-3-yl)-2-(1-methyl-1H-pyrazol-4-yl)pyrazolo[5,1-b]thiazole-7-carboxamide

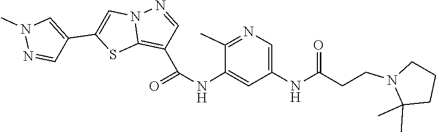 [SMILES: Cc1ncc(NC(=O)CCN2CCCC2(C)C)cc1NC(=O)c1cnn2cc(-c3cnn(C)c3)sc12]

### Step a: tert-butyl (6-methyl-5-(2-(1-methyl-1H-pyrazol-4-yl)pyrazolo[5,1-b]thiazole-7-carboxamido) pyridin-3-yl)carbamate

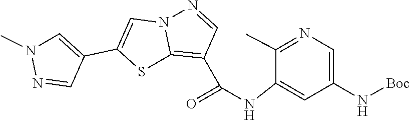 [SMILES: Cc1ncc(NC(=O)OC(C)(C)C)cc1NC(=O)c1cnn2cc(-c3cnn(C)c3)sc12]

To a solution of tert-butyl (5-(2-bromopyrazolo[5,1-b]thiazole-7-carboxamido)-6-methylpyridin-3-yl)carbamate (12 g, 26 mmol), 1-methyl-4-(4,4,5,5-tetramethyl-1,3,2-dioxaborolan-2-yl)-1H-pyrazole (7.2 g, 33.3 mmol) and Cs₂CO₃ (24 g, 79.20 mmol) in dioxane/H₂O (1500 mL, 4/1) was added PdCl₂(dppf)·CH₂Cl₂ (6.6 g, 48.6 mmol) at room-temperature under an atmosphere of nitrogen. The reaction vessel was gradually warmed to 100° C. over the course of 10 min, after which time stirring was continued for 12 hours. The reaction mixture was concentrated to dryness in vacuum give black solid. The black solid was treated with dichloromethane/methanol (50 mL, 5/1). The reaction mixture was filtered and the filter cake was rinsed with 50 mL of dichloromethane. The filtrate was collected, dried in vacuum to give a black solid. The black solid was subjected to column chromatography over silica gel (gradient elution: 0-10% methanol in dichloromethane). The pure fractions were collected and concentrated to dryness in vacuum to give tert-butyl (6-methyl-5-(2-(1-methyl-1H-pyrazol-4-yl)pyrazolo[5,1-b]thiazole-7-carboxamido) pyridin-3-yl)carbamate (6.5 g, 13.20 mmol, 50% yield) as a brown solid. LCMS (ESI): mass calcd. for C₂₁H₂₃N₇O₃S, 453.2; m/z found, 454.1 [M+H]⁺.

### Step b: N-(5-amino-2-methylpyridin-3-yl)-2-(1-methyl-1H-pyrazol-4-yl)pyrazolo[5,1-b]thiazole-7-carboxamide

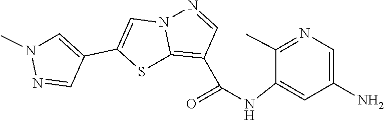 [SMILES: Cc1ncc(N)cc1NC(=O)c1cnn2cc(-c3cnn(C)c3)sc12]

To the solution of tert-butyl (6-methyl-5-(2-(1-methyl-1H-pyrazol-4-yl)pyrazolo[5,1-b]thiazole-7-carboxamido) pyridin-3-yl)carbamate (6.5 g, 14.332 mmol) in DCM (50 mL) was added HCl/dioxane (32.5 mL, 130 mmol). The resulting mixture was stirred at r.t. for 16 hours. The reaction mixture was concentrated to dryness to give N-(5-amino-2-methylpyridin-3-yl)-2-(1-methyl-1H-pyrazol-4-yl)pyrazolo[5,1-b]thiazole-7-carboxamide (6 g, 92%) as a brown solid (HCl salt). LCMS (ESI): mass calcd. for C₁₆H₁₅N₇OS, 353.1; m/z found, 354.2[M+H]⁺.

### Step c: N-(5-(3-chloropropanamido)-2-methylpyridin-3-yl)-2-(1-methyl-11H-pyrazol-4-yl)pyrazolo[5,1-b]thiazole-7-carboxamide

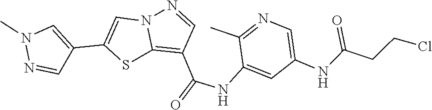 [SMILES: Cc1ncc(NC(=O)CCCl)cc1NC(=O)c1cnn2cc(-c3cnn(C)c3)sc12]

To a solution of N-(5-amino-2-methylpyridin-3-yl)-2-(1-methyl-1H-pyrazol-4-yl)pyrazolo[5,1-b]thiazole-7-carboxamide (5.0 g, 12.8 mmol) in CHCl₃ (100 mL) was added saturated aqueous NaHCO₃ (100 mL), The reaction mixture was stirred at 0° C. Then 3-chloropropanoyl chloride (5.0 mL, 51.2 mmol) was added dropwise, and the mixture was stirred at room temperature overnight. The reaction mixture became cloudy. Color changes (brown to black) were observed. Water (1000 mL) was added to the mixture and the mixture was extracted with ethyl acetate (1000 mL×4). The organic layer was dried over anhydrous Na₂SO₄, filtered, and concentrated to dryness in vacuo to give N-(5-(3-chloropropanamido)-2-methylpyridin-3-yl)-2-(1-methyl-1H-pyrazol-4-yl)pyrazolo[5,1-b]thiazole-7-carboxamide (5.0 g, 74%) as a gray solid. LCMS (ESI): mass calcd. for C₁₉H₁₈ClN₇O₂S, 443.9; m/z found, 444.0 [M+H]⁺.

### Step d: N-(5-(3-(2,2-dimethylpyrrolidin-1-yl)propanamido)-2-methylpyridin-3-yl)-2-(1-methyl-1H-pyrazol-4-yl)pyrazolo[5,1-b]thiazole-7-carboxamide

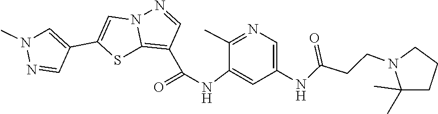 [SMILES: Cc1ncc(NC(=O)CCN2CCCC2(C)C)cc1NC(=O)c1cnn2cc(-c3cnn(C)c3)sc12]

To a solution of N-(5-(3-chloropropanamido)-2-methylpyridin-3-yl)-2-(1-methyl-1H-pyrazol-4-yl)pyrazolo[5,1-b]thiazole-7-carboxamide (1.0 g, 1.91 mmol) and TEA(1.5 mL, 10.8 mmol) in DMSO (10 mL). Then 2,2-dimethylpyrrolidine (650 mg, 6.554 mmol) was added to the solution. The reaction mixture was stirred at 60° C. overnight. The mixture was filtered through Celite. The filtrate was concentrated to afford crude product. The crude product was purified by preparative high-performance liquid chromatography over Column: YMC Triart C18 250*50 mm*7 um to give the title compound N-(5-(3-(2,2-dimethylpyrrolidin-1-yl)propanamido)-2-methylpyridin-3-yl)-2-(1-methyl-1H-pyrazol-4-yl)pyrazolo[5,1-b]thiazole-7-carboxamide (884 mg, 90%) as white solid. LCMS (ESI): mass calcd. for C₂₅H₃₀N₈O₂S, 506.6; m/z found, 507.2[M+H]⁺. ¹H NMR (400 MHz, METHANOL-d₄) δ 8.56 (d, J=2.27 Hz, 1H), 8.41 (s, 1H), 8.21-8.27 (m, 2H), 8.05 (s, 1 H), 7.85 (s, 1H), 3.97 (s, 3H), 2.94 (br s, 4H), 2.66 (br s, 2H), 2.51 (s, 3H), 1.90 (br s, 2H), 1.80 (br s, 2H), 1.16 (br s, 6H).

### Example 43. N-(5-(3-(2-azabicyclo[2.2.2]octan-2-yl)propanamido)-2-methyl pyridin-3-yl)-2-(1-methyl-1H-pyrazol-4-yl)pyrazolo[5,1-b]thiazole-7-carboxamide

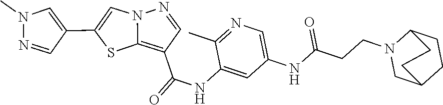 [SMILES: Cc1ncc(NC(=O)CCN2CC3CCC2CC3)cc1NC(=O)c1cnn2cc(-c3cnn(C)c3)sc12]

To a solution of N-(5-(3-chloropropanamido)-2-methylpyridin-3-yl)-2-(1-methyl-1H-pyrazol-4-yl)pyrazolo[5,1-b]thiazole-7-carboxamide (250 mg, 0.56 mmol), 2-azabicyclo[2.2.2]octane (325 mg, 2.2 mmol) in Dimethyl sulfoxide (5 mL) was added 2,3,4,6,7,8,9,10-octahydropyrimido[1,2-a]azepine (125 mg, 0.82 mmol). The mixture stirred at 60° C. for 12 h and then concentrated under vacuum to give the crude product, which was purified by preparative high-performance liquid chromatography over Column: Welch Xtimate C18 150*30 mm*5 um to give the title compound N-(5-(3-(2-azabicyclo[2.2.2]octan-2-yl)propanamido)-2-methylpyridin-3-yl)-2-(1-methyl-1H-pyrazol-4-yl)pyrazolo[5,1-b]thiazole-7-carboxamide (100 mg, 31.7%) as a white solid. LCMS (ESI): mass calcd. for C₂₆H₃₀N₈O₂S, 518.6; m/z found, 519.3 [M+H]⁺. ¹H NMR (400 MHz, METHANOL-d₄) δ 8.57 (d, J=2.3 Hz, 1H), 8.39 (s, 1H), 8.26-8.23 (m, 2H), 8.04 (s, 1H), 7.83 (s, 1H), 3.95 (s, 3H), 3.50-3.39 (m, 2H), 3.36-3.32 (m, 1H), 3.22 (br d, J=3.5 Hz, 2H), 2.88-2.82 (m, 2H), 2.51 (s, 3H), 2.14 (br d, J=10.5 Hz, 2H), 1.92 (br s, 1H), 1.83-1.72 (m, 6H).

### Intermediate B-1

### N-(5-amino-6-methylpyridin-3-yl)-2-(2,2-dimethylpyrrolidin-1-yl)acetamide

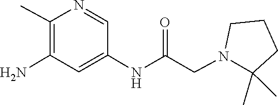 [SMILES: Cc1ncc(NC(=O)CN2CCCC2(C)C)cc1N]

### Step a: tert-butyl (5-(2-chloroacetamido)-2-methylpyridin-3-yl)carbamate

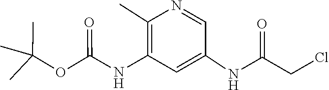 [SMILES: Cc1ncc(NC(=O)CCl)cc1NC(=O)OC(C)(C)C]

To a solution of tert-butyl (5-amino-2-methylpyridin-3-yl)carbamate (1.0 g, 4.48 mmol) and pyridine (710 mg, 8.98 mmol) in DCM (10 mL) at 0° C. under nitrogen was added a solution of chloroacetyl chloride (560 mg, 4.96 mmol) in DCM (10 mL). The reaction was warmed up to rt and stirred at rt for 1 h. The reaction was quenched with H₂O (30 mL) and the resulting mixture was extracted with DCM (100 mL×3). The organic layers were combined, dried over anhydrous sodium sulfate, filtered, and concentrated. The residue obtained was purified by silica gel chromatography (0-65% ethyl acetate/petroleum ether) to afford tert-butyl (5-(2-chloroacetamido)-2-methylpyridin-3-yl)carbamate as a white solid (1.1 g, 82% yield). LCMS (ESI): mass calcd. for C₁₃H₁₈ClN₃O₃, 299.10 m/z; found, 300.05 [M+H]⁺.

### Step b: tert-butyl (5-(2-(2,2-dimethylpyrrolidin-1-yl)acetamido)-2-methylpyridin-3-yl)carbamate

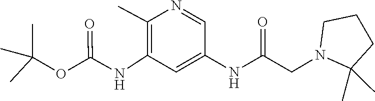 [SMILES: Cc1ncc(NC(=O)CN2CCCC2(C)C)cc1NC(=O)OC(C)(C)C]

A mixture of 2,2-dimethylpyrrolidine hydrochloride (5.0 g, 36.9 mmol) and K₂CO₃ (9.2 g, 66.6 mmol) in ACN (100 mL) was stirred for 20 min before tert-butyl (5-(2-chloroacetamido)-2-methylpyridin-3-yl)carbamate (10.0 g, 33.4 mmol) was added. The resulting mixture was stirred at rt overnight. The reaction mixture was concentrated. The residue obtained was purified by silica gel chromatography (0-10% MeOH/DCM) to afford tert-butyl (5-(2-(2,2-dimethylpyrrolidin-1-yl)acetamido)-2-methylpyridin-3-yl)carbamate as an off-white solid (8.6 g, 71% yield). LCMS (ESI) calcd. for C₁₉H₃₀N₄O₃: 362.23 m/z, found: 363.15 [M+H]⁺.

### Step c: N-(5-amino-6-methylpyridin-3-yl)-2-(2,2-dimethylpyrrolidin-1-yl)acetamide

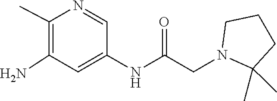

A mixture of tert-butyl (5-(2-(2,2-dimethylpyrrolidin-1-yl)acetamido)-2-methylpyridin-3-yl)carbamate (11.0 g, 30.3 mmol) in 4M HCl in 1,4-dioxane (80 mL) was stirred at rt for 1 h. The mixture was concentrated under reduced pressure. To the resulting residue was added Et₂O (300 mL). The mixture was stirred for 10 min before it was filtered. The solid was washed with Et₂O (50 mL×3) and was dried under vacuum to afford N-(5-amino-6-methylpyridin-3-yl)-2-(2,2-dimethylpyrrolidin-1-yl)acetamide hydrochloride as yellow solid (7.94 g, 98% yield). LCMS (ESI) calcd. for C₁₄H₂₂N₄O: 262.18 m/z, found: 263.10 [M+H]⁺. ¹H NMR (400 MHz, DMSO-d6) δ 8.20 (d, J=2.1 Hz, 1H), 7.70 (d, J=2.0 Hz, 1H), 4.30-4.40 (m, 1H), 3.99-4.08 (m, 1H), 3.80-2.83 (m, 1H), 3.30-3.38 (m, 1H), 2.45 (s, 3H), 1.80-2.11 (m, 4H), 1.45 (s, 3H), 1.22 (s, 3H).

### Intermediate B-2

### (S)—N-(5-amino-6-methylpyridin-3-yl)-2-(2-methylpyrrolidin-1-yl)acetamide

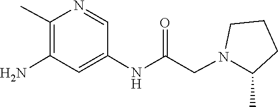 [SMILES: Cc1ncc(NC(=O)CN2CCC[C@@H]2C)cc1N]

(S)—N-(5-amino-6-methylpyridin-3-yl)-2-(2-methylpyrrolidin-1-yl)acetamide was prepared following similar procedure as that of Intermediate B-1, using (S)-2-methylpyrrolidine instead of 2,2-dimethylpyrrolidine. LCMS (ESI) calcd. for C₁₃H₂₀N₄O: 248.16 m/z, found: 249.10 [M+H]⁺. ¹H NMR (300 MHz, DMSO-d6) δ 8.22 (d, J=2.0 Hz, 1H), 7.65 (d, J=2.1 Hz, 1H), 4.30-4.40 (m, 1H), 4.08-4.14 (m, 1H), 3.75-3.85 (m, 1H), 3.52-3.63 (m, 1H), 3.15-3.30 (m, 1H), 2.46 (s, 3H), 2.20-2.35 (m, 1H), 1.90-2.05 (m, 2H), 1.59-1.80 (m, 1H), 1.40 (d, J=6.4 Hz, 3H).

### Intermediate B-3

### (R)—N-(5-amino-6-methylpyridin-3-yl)-2-(2-methylpyrrolidin-1-yl)acetamide

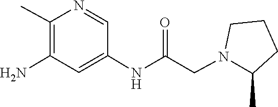 [SMILES: Cc1ncc(NC(=O)CN2CCC[C@H]2C)cc1N]

(R)—N-(5-amino-6-methylpyridin-3-yl)-2-(2-methylpyrrolidin-1-yl)acetamide was prepared following similar procedure as that of Intermediate B-1, using (R)-2-methylpyrrolidine instead of 2,2-dimethylpyrrolidine. LCMS (ESI) calcd. for C₁₃H₂₀N₄O: 248.16 m/z, found: 249.10 [M+H]⁺. ¹H NMR (300 MHz, DMSO-d6) δ 8.22 (d, J=2.1 Hz, 1H), 7.65 (d, J=2.1 Hz, 1H), 4.30-4.42 (m, 1H), 4.02-4.12 (m, 1H), 3.70-3.82 (m, 1H), 3.45-3.60 (m, 1H), 3.10-3.30 (m, 1H), 2.46 (s, 3H), 2.15-2.28 (m, 1H), 1.90-2.10 (m, 2H), 1.57-1.79 (m, 1H), 1.30-1.42 (m, 3H).

### Intermediate B-4

### N-(5-amino-6-methylpyridin-3-yl)-2-(3,3-dimethylazetidin-1-yl)acetamide

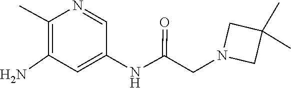

N-(5-amino-6-methylpyridin-3-yl)-2-(3,3-dimethylazetidin-1-yl)acetamide was prepared following similar procedure as that of Intermediate B-1, using 3,3-dimethylazetidine instead of 2,2-dimethylpyrrolidine. LCMS (ESI) calcd. for C₁₃H₂₀N₄O: 248.16 m/z, found: 249.15 [M+H]⁺. ¹H NMR (400 MHz, DMSO-d6) δ 8.14 (d, J=2.0 Hz, 1H), 7.69 (d, J=2.1 Hz, 1H), 4.34 (s, 2H), 3.88-3.93 (m, 4H), 2.44 (s, 3H), 1.35 (s, 3H), 1.23 (s, 3H).

### Example 44: N-(5-(2-(3-oxa-8-azabicyclo[3.2.1]octan-8-yl)acetamido)-2-methylpyridin-3-yl)-2-(1-methyl-1H-pyrazol-4-yl)pyrazolo[5,1-b]thiazole-7-carboxamide

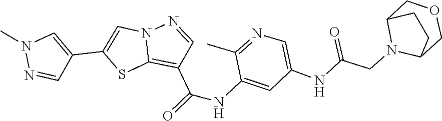 [SMILES: Cc1ncc(NC(=O)CN2C3CCC2COC3)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (150 mg, 0.23 mmol) and 3-oxa-8-azabicyclo[3.2.1]octane hydrochloride (41 mg, 0.27 mmol) in DMF (2 mL) was added K₂CO₃ (94 mg, 0.68 mmol) and NaI (20 mg, 0.14 mmol) at room temperature. The mixture was stirred at 50° C. for 1.5 h. The mixture was filtered through Celite, rinsed with DMF (3 mL). The filtrate was concentrated to afford crude product. The crude product was purified by preparative high-performance liquid chromatography over column: Phenomenex Gemini-NX C18 75*30 mm*3 um to give the title compound N-(5-(2-(3-oxa-8-azabicyclo[3.2.1]octan-8-yl)acetamido)-2-methylpyridin-3-yl)-2-(1-methyl-1H-pyrazol-4-yl)pyrazolo[5,1-b]thiazole-7-carboxamide (40 mg, 35%) as a white solid. LCMS (ESI): mass calcd. for C₂₄H₂₆N₈O₃S, 506.6; m/z found, 507.2[M+H]⁺. ¹H NMR (400 MHz, DMSO-d₆) δ 10.04 (s, 1H), 9.98 (s, 1H), 8.70 (s, 1H), 8.65 (s, 1H), 8.58 (s, 1H), 8.26 (s, 2H), 7.95 (s, 1H), 3.95 (s, 3H), 3.82 (br d, J=10.14 Hz, 2H), 3.51 (br s, 2H), 3.18 (br s, 2H), 3.13 (s, 2H), 2.47 (s, 3H), 1.93 (br d, J=4.63 Hz, 2H), 1.77-1.88 (m, 2H).

### Example 45: N-(5-(2-(6-oxa-3-azabicyclo[3.1.1]heptan-3-yl)acetamido)-2-methylpyridin-3-yl)-2-(1-methyl-1H-pyrazol-4-yl)pyrazolo[5,1-b]thiazole-7-carboxamide

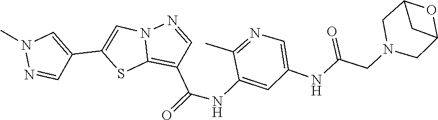 [SMILES: Cc1ncc(NC(=O)CN2CC3CC(C2)O3)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (150 mg, 0.23 mmol), 6-oxa-3-azabicyclo[3.1.1]heptane hydrochloride (37 mg, 0.27 mmol), potassium carbonate (94 mg, 0.68 mmol) in N,N-dimethylformamide (2 mL) was added sodium iodide (20 mg, 0.14 mmol). The mixture was stirred at 50° C. for 2 hours. The reaction mixture was concentrated under vacuum to give the crude product, which was purified by preparative high-performance liquid chromatography over column: Phenomenex Gemini-NX C18 75*30 mm*3 um to give the title compound N-(5-(2-(6-oxa-3-azabicyclo[3.1.1]heptan-3-yl)acetamido)-2-methylpyridin-3-yl)-2-(1-methyl-1H-pyrazol-4-yl)pyrazolo[5,1-b]thiazole-7-carboxamide (45 mg, 40%) as a white solid. LCMS (ESI): mass calcd. for C₂₃H₂₄N₈O₃S, 492.6; m/z found, 493.2 [M+H]⁺. ¹H NMR (400 MHz, DMSO-d₆) δ 9.91 (s, 1H), 9.86 (s, 1H), 8.55 (s, 2H), 8.47 (s, 1H), 8.16 (s, 1H), 8.13 (d, J=1.98 Hz, 1H), 7.84 (s, 1H), 4.41 (d, J=6.17 Hz, 2H), 3.84 (s, 3H), 3.37 (s, 2H), 3.08 (d, J=10.80 Hz, 2H), 2.78-2.89 (m, 3H), 2.33-2.42 (m, 4H).

### Example 46: N-(5-(2-(2-oxa-7-azaspiro[3.5]nonan-7-yl)acetamido)-2-methylpyridin-3-yl)-2-(1-methyl-1H-pyrazol-4-yl)pyrazolo[5,1-b]thiazole-7-carboxamide

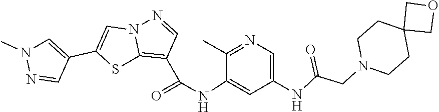 [SMILES: Cc1ncc(NC(=O)CN2CCC3(CC2)COC3)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (150 mg, 0.23 mmol), 2-oxa-7-azaspiro[3.5]nonane (35 mg, 0.27 mmol), and potassium carbonate (94 mg, 0.68 mmol) in DMF (2 mL) was added sodium iodide (20 mg, 0.14 mmol). The resulting mixture was stirred at 50° C. for 2 h and then the reaction mixture was filtered and the filtrate was purified by preparative high-performance liquid chromatography over column: Phenomenex Gemini-NX C18 75*30 mm*3 um to give the title compound N-(5-(2-(2-oxa-7-azaspiro[3.5]nonan-7-yl)acetamido)-2-methylpyridin-3-yl)-2-(1-methyl-1H-pyrazol-4-yl)pyrazolo[5,1-b]thiazole-7-carboxamide (47 mg, 40%) as a white solid. LCMS (ESI): mass calcd. for C₂₅H₂₈N₈O₃S, 520.6; m/z found, 521.2 [M+H]⁺. ¹H NMR (400 MHz, DMSO-d₆) δ 9.87 (d, J=3.31 Hz, 2H), 8.50-8.58 (m, 2H), 8.47 (s, 1H), 8.16 (s, 1H), 8.12 (d, J=1.98 Hz, 1H), 7.84 (s, 1H), 4.25 (s, 4H), 3.84 (s, 3H), 3.06 (s, 2H), 2.36 (s, 7H), 1.80 (br t, J=5.29 Hz, 4H).

### Example 47: N-(5-(2-(7-oxa-4-azaspiro[2.5]octan-4-yl)acetamido)-2-methylpyridin-3-yl)-2-(1-methyl-1H-pyrazol-4-yl)pyrazolo[5,1-b]thiazole-7-carboxamide

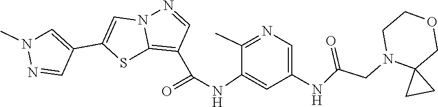 [SMILES: Cc1ncc(NC(=O)CN2CCOCC23CC3)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (100 mg, 0.23 mmol), 7-oxa-4-azaspiro[2.5]octane hydrochloride (40 mg, 0.27 mmol), and potassium carbonate (94 mg, 0.68 mmol) in DMF (2 mL) was added sodium iodide (20 mg, 0.14 mmol). The resulting mixture was stirred at 50° C. for 2 h and then the reaction mixture was filtered and the filtrate was purified by preparative high-performance liquid chromatography over column: Boston Prime C18 150*30 mm*5 um to give the title compound: N-(5-(2-(7-oxa-4-azaspiro[2.5]octan-4-yl)acetamido)-2-methylpyridin-3-yl)-2-(1-methyl-1H-pyrazol-4-yl)pyrazolo[5,1-b]thiazole-7-carboxamide (31 mg, 26%) as a white solid. LCMS (ESI): mass calcd. for C₂₄H₂₆N₈O₃S, 506.6; m/z found, 507.2 [M+H]⁺. ¹H NMR (400 MHz, CHLOROFORM-d) δ 8.96 (s, 1H), 8.68 (d, J=2.43 Hz, 1H), 8.47 (d, J=2.21 Hz, 1H), 8.03 (s, 1H), 7.81 (s, 1H), 7.69 (s, 1H), 7.60 (s, 1H), 7.31 (s, 1H), 3.97 (s, 3H), 3.73-3.81 (m, 2H), 3.50 (br s, 2H), 3.47 (s, 2H), 3.01 (br s, 2H), 2.57 (s, 3H), 0.78 (s, 2H), 0.63 (s, 2H).

### Example 48: N-(5-(2-(5H-pyrrolo[3,4-b]pyridin-6 (7H)-yl)acetamido)-2-methylpyridin-3-yl)-2-(1-methyl-1H-pyrazol-4-yl)pyrazolo[5,1-b]thiazole-7-carboxamide

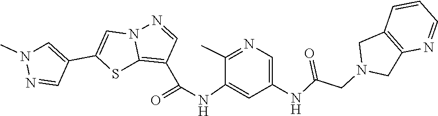 [SMILES: Cc1ncc(NC(=O)CN2Cc3cccnc3C2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (100 mg, 0.23 mmol), 6,7-dihydro-5H-pyrrolo[3,4-b]pyridine (33 mg, 0.27 mmol), and potassium carbonate (94 mg, 0.68 mmol) in DMF (2 mL) was added sodium iodide (20 mg, 0.14 mmol). The resulting mixture was stirred at 50° C. for 2 h and then the reaction mixture was filtered and the filtrate was purified by preparative high-performance liquid chromatography over column: Boston Prime C18 150*30 mm*5 um to give the title compound: N-(5-(2-(5H-pyrrolo[3,4-b]pyridin-6 (7H)-yl)acetamido)-2-methylpyridin-3-yl)-2-(1-methyl-1H-pyrazol-4-yl)pyrazolo[5,1-b]thiazole-7-carboxamide (66 mg, 57%) as a white solid. LCMS (ESI): mass calcd. for C₂₅H₂₃N₉O₂S, 513.6; m/z found, 514.2[M+H]⁺. ¹H NMR (400 MHz, CHLOROFORM-d) δ 9.09 (br s, 1H), 8.75 (br s, 1H), 8.53 (br s, 1H), 8.46 (br s, 1H), 8.03 (br s, 1H), 7.80 (s, 1H), 7.68 (s, 1H), 7.59 (s, 1H), 7.55 (br d, J=6.39 Hz, 1H), 7.36 (br s, 1H), 7.18 (br s, 1H), 4.20 (br s, 4H), 3.96 (s, 3H), 3.62 (s, 2H), 2.58 (s, 3H).

### Example 49: N-(5-(2-(3,3-difluoropiperidin-1-yl)acetamido)-2-methylpyridin-3-yl)-2-(1-methyl-1H-pyrazol-4-yl)pyrazolo[5,1-b]thiazole-7-carboxamide

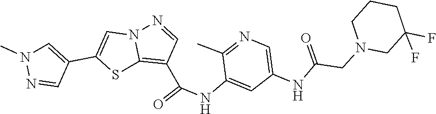 [SMILES: Cc1ncc(NC(=O)CN2CCCC(F)(F)C2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (120 mg, 0.23 mmol), 3,3-difluoropiperidine hydrochloride (44 mg, 0.28 mmol), potassium carbonate (97 mg, 0.70 mmol) in N,N-dimethylformamide (2 mL) was added sodium iodide (21 mg, 0.14 mmol), The mixture was stirred at 50° C. for 2 hours. The reaction mixture was concentrated under vacuum to give the crude product, which was purified by preparative high-performance liquid chromatography over column: Welch Xtimate C18 150*25 mm*5 um and Supercritical Fluid Chromatography over column: DAICEL CHIRALPAK AS (250 mm*30 mm, 10 um) to give the title compound N-(5-(2-(3,3-difluoropiperidin-1-yl)acetamido)-2-methylpyridin-3-yl)-2-(1-methyl-1H-pyrazol-4-yl)pyrazolo[5,1-b]thiazole-7-carboxamide (54 mg, 49%) as a white solid. LCMS (ESI): mass calcd. for C₂₃H₂₄F₂N₈O₂S, 514.6; m/z found, 515.3 [M+H]⁺. ¹H NMR (400 MHz, CHLOROFORM-d) δ 9.12 (s, 1H), 8.56 (dd, J=13.12, 2.32 Hz, 2H), 8.05 (s, 1H), 7.80 (s, 1H), 7.68 (s, 1H), 7.60 (s, 1H), 7.43 (s, 1H), 3.96 (s, 3H), 3.24 (s, 2H), 2.80 (br t, J=10.80 Hz, 2H), 2.66 (br s, 2H), 2.56 (s, 3H), 1.96 (br dd, J=13.01, 6.39 Hz, 2H), 1.85-1.91 (m, 2H).

### Example 50: (R)-2-(1-methyl-1H-pyrazol-4-yl)-N-(2-methyl-5-(2-(3-methylmorpholino)acetamido)pyridin-3-yl)pyrazolo[5,1-b]thiazole-7-carboxamide

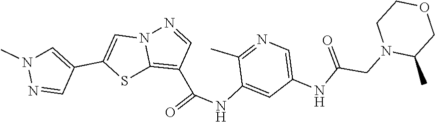 [SMILES: Cc1ncc(NC(=O)CN2CCOC[C@H]2C)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (70 mg, 0.16 mmol), (R)-3-methylmorpholine (19 mg, 0.19), and potassium carbonate (65 mg, 0.47 mmol) in DMF (1.5 mL) was added sodium iodide (14 mg, 0.10 mmol). The resulting mixture was stirred at 50° C. for 2 h and then the reaction mixture was filtered and the filtrate was purified by preparative high-performance liquid chromatography over column: Boston Prime C18 150*30 mm*5 um to give the title compound (R)-2-(1-methyl-1H-pyrazol-4-yl)-N-(2-methyl-5-(2-(3-methylmorpholino)acetamido)pyridin-3-yl)pyrazolo[5,1-b]thiazole-7-carboxamide (30 mg, 38%) as a white solid. LCMS (ESI): mass calcd for C₂₃H₂₆N₈O₃S, 494.6; m/z found, 495.2 [M+H]⁺. ¹H NMR (400 MHz, CHLOROFORM-d) δ 9.13 (s, 1H), 8.66 (d, J=2.21 Hz, 1H), 8.48 (d, J=1.98 Hz, 1H), 8.07 (s, 1H), 7.80 (s, 1H), 7.68 (s, 1H), 7.60 (s, 1H), 7.46 (s, 1H), 3.96 (s, 3H), 3.81-3.90 (m, 1H), 3.68-3.80 (m, 2H), 3.30-3.48 (m, 2H), 2.75-3.02 (m, 2H), 2.57-2.66 (m, 2H), 2.56 (s, 3H), 1.01 (d, J=6.39 Hz, 3H).

### Example 51: N-(5-(2-((3R,5R)-3,5-dimethylmorpholino)acetamido)-2-methylpyridin-3-yl)-2-(1-methyl-1H-pyrazol-4-yl)pyrazolo[5,1-b]thiazole-7-carboxamide

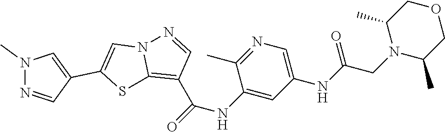 [SMILES: Cc1ncc(NC(=O)CN2[C@H](C)COC[C@H]2C)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (120 mg, 0.28 mmol), (3R,5R)-3,5-dimethylmorpholine hydrochloride (51 mg, 0.34 mmol), and potassium carbonate (116 mg, 0.84 mmol) in N,N-dimethylformamide (2 mL) was added sodium iodide (25 mg, 0.17 mmol), the mixture was stirred at 50° C. for 2 hours. The reaction mixture was concentrated under vacuum to give the crude product, which was purified by preparative high-performance liquid chromatography over column: Phenomenex Gemini-NX 80*40 mm*3 um to give the title compound N-(5-(2-((3R,5R)-3,5-dimethylmorpholino)acetamido)-2-methylpyridin-3-yl)-2-(1-methyl-1H-pyrazol-4-yl)pyrazolo[5,1-b]thiazole-7-carboxamide (33 mg, 24%) as a gray solid. LCMS (ESI): mass calcd. for C₂₄H₂₈N₈O₃S, 508.6; m/z found, 509.2 [M+H]⁺. ¹H NMR (400 MHz, CHLOROFORM-d) δ 9.28 (s, 1H), 8.65 (d, J=2.21 Hz, 1H), 8.51 (d, J=2.20 Hz, 1H), 8.04 (s, 1H), 7.81 (s, 1H), 7.69 (s, 1H), 7.60 (s, 1H), 7.34 (br s, 1H), 3.96 (s, 3H), 3.80 (dd, J=11.25, 2.87 Hz, 2H), 3.48 (br s, 2H), 3.15-3.33 (m, 2H), 2.96 (td, J=6.28, 3.09 Hz, 2H), 2.57 (s, 3H), 1.06 (br d, J=6.17 Hz, 6H).

### Example 52: N-(5-(2-((2S,6S)-2,6-dimethylmorpholino)acetamido)-2-methylpyridin-3-yl)-2-(1-methyl-1H-pyrazol-4-yl)pyrazolo[5,1-b]thiazole-7-carboxamide

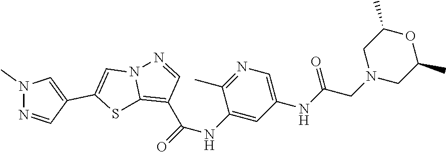

To a solution of N-(5-(2-chloroacetamido)-2-methylpyridin-3-yl)-2-(1-methyl-1H-pyrazol-4-yl)pyrazolo[5,1-b]thiazole-7-carboxamide (100 mg, 0.23 mmol), (2S,6S)-2,6-dimethylmorpholine (32 mg, 0.28 mmol), and potassium carbonate (96 mg, 0.70 mmol) in DMF (2 mL) was added sodium iodide (21 mg, 0.14 mmol). The resulting mixture was stirred at 50° C. for 2 h and then the reaction mixture was filtered and the filtrate was purified by preparative high-performance liquid chromatography over column: Welch Xtimate C18 150*30 mm*5 um to give the title compound: N-(5-(2-((2S,6S)-2,6-dimethylmorpholino)acetamido)-2-methylpyridin-3-yl)-2-(1-methyl-1H-pyrazol-4-yl)pyrazolo[5,1-b]thiazole-7-carboxamide (38 mg, 31%) as a white solid. LCMS (ESI): mass calcd. for C₂₄H₂₈N₈O₃S, 508.6; m/z found, 509.3 [M+H]⁺. ¹H NMR (400 MHz, CHLOROFORM-d) δ 9.09 (s, 1H), 8.57 (dd, J=13.78, 2.09 Hz, 2H), 8.03 (s, 1H), 7.80 (s, 1H), 7.69 (s, 1H), 7.60 (s, 1H), 7.31 (s, 1H), 4.11 (td, J=6.06, 3.31 Hz, 2H), 3.96 (s, 3H), 3.01-3.19 (m, 2H), 2.63 (dd, J=11.14, 2.98 Hz, 2H), 2.56 (s, 3H), 2.34 (dd, J=11.03, 5.73 Hz, 2H), 1.33 (s, 6H).

### Example 53: N-(5-(2-((2R,6S)-2,6-dimethylmorpholino)acetamido)-2-methylpyridin-3-yl)-2-(1-methyl-1H-pyrazol-4-yl)pyrazolo[5,1-b]thiazole-7-carboxamide

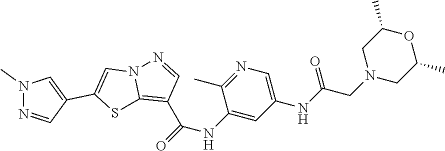

To a solution of N-(5-(2-chloroacetamido)-2-methylpyridin-3-yl)-2-(1-methyl-1H-pyrazol-4-yl)pyrazolo[5,1-b]thiazole-7-carboxamide (100 mg, 0.23 mmol), (2S,6R)-2,6-dimethylmorpholine (32 mg, 0.28 mmol), and potassium carbonate (96 mg, 0.70 mmol) in DMF (2 mL) was added sodium iodide (21 mg, 0.14 mmol). The resulting mixture was stirred at 50° C. for 2 h and then the reaction mixture was filtered and the filtrate was purified by preparative high-performance liquid chromatography over column: Phenomenex Gemini-NX C18 75*30 mm*3 um to give the title compound: N-(5-(2-((2R,6S)-2,6-dimethylmorpholino)acetamido)-2-methylpyridin-3-yl)-2-(1-methyl-1H-pyrazol-4-yl)pyrazolo[5,1-b]thiazole-7-carboxamide (52 mg, 42%) as a white solid. LCMS (ESI): mass calcd. for C₂₄H₂₈N₈O₃S, 508.6; m/z found, 509.3 [M+H]⁺. ¹H NMR (400 MHz, CHLOROFORM-d) δ 8.98 (s, 1H), 8.63 (d, J=2.20 Hz, 1H), 8.46 (d, J=2.20 Hz, 1H), 7.98 (s, 1H), 7.76 (s, 1H), 7.63 (s, 1H), 7.55 (s, 1H), 7.27 (s, 1H), 3.91 (s, 3H), 3.66-3.76 (m, 2H), 3.07 (s, 2H), 2.70 (br d, J=10.80 Hz, 2H), 2.52 (s, 3H), 1.97 (t, J=10.91 Hz, 2H), 1.13 (d, J=6.17 Hz, 6H).

### Example 54: (R)-2-(1-methyl-1H-pyrazol-4-yl)-N-(2-methyl-5-(2-(2-methylmorpholino)acetamido)pyridin-3-yl)pyrazolo[5,1-b]thiazole-7-carboxamide

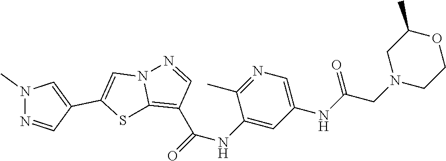 [SMILES: Cc1ncc(NC(=O)CN2CCO[C@H](C)C2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (120 mg, 0.25 mmol), (R)-2-methylmorpholine hydrochloride (41 mg, 0.30 mmol), potassium carbonate (103 mg, 0.75 mmol) in N,N-dimethylformamide (2 mL) was added sodium iodide (22 mg, 0.15 mmol), The mixture was stirred at 50° C. for 2 hours. The reaction mixture was concentrated under vacuum to give the crude product, which was purified by preparative high-performance liquid chromatography over column: Boston Prime C18 150*30 mm*5 um and Supercritical Fluid Chromatography over column: DAICEL CHIRALCEL OD-H (250 mm*30 mm, 5 um) to give the title compound (R)-2-(1-methyl-1H-pyrazol-4-yl)-N-(2-methyl-5-(2-(2-methylmorpholino)acetamido)pyridin-3-yl)pyrazolo[5,1-b]thiazole-7-carboxamide (31 mg, 30%) as a white solid. LCMS (ESI): mass calcd. for C₂₃H₂₆N₈O₃S, 494.6; m/z found, 495.2[M+H]⁺. ¹H NMR (400 MHz, DMSO-d₆) δ 9.96 (s, 1H), 9.89 (s, 1H), 8.59 (s, 2H), 8.51 (s, 1H), 8.20 (s, 1H), 8.16 (d, J=2.26 Hz, 1H), 7.89 (s, 1H), 3.89 (s, 3H), 3.75 (br d, J=9.18 Hz, 1H), 3.57-3.66 (m, 2H), 3.16 (s, 2H), 2.70-2.80 (m, 2H), 2.41 (s, 3H), 1.90-2.27 (m, 2H), 1.05 (d, J=6.32 Hz, 3H).

### Example 55: 2-(1-methyl-1H-pyrazol-4-yl)-N-(2-methyl-5-(2-morpholinoacetamido)pyridin-3-yl)pyrazolo[5,1-b]thiazole-7-carboxamide

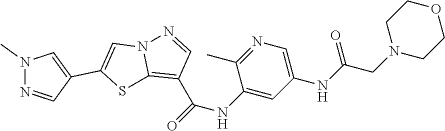 [SMILES: Cc1ncc(NC(=O)CN2CCOCC2)cc1NC(=O)c1cnn2cc(-c3cnn(C)c3)sc12]

To a mixture of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.21 mmol), K₂CO₃ (87 mg, 0.63 mmoL) and NaI (19 mg, 0.13 mmol) in DMF (2 mL) was added morpholine (22 mg, 0.25 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Phenomenex Gemini-NX 80*40 mm*3 um to give the title compound 2-(1-methyl-1H-pyrazol-4-yl)-N-(2-methyl-5-(2-morpholinoacetamido)pyridin-3-yl)pyrazolo[5,1-b]thiazole-7-carboxamide (42 mg, 41%) as a brown solid. LCMS (ESI): mass calcd. for C₂₂H₂₄N₈O₃S, 480.5; m/z found, 481.2 [M+H]⁺. ¹H NMR (400 MHz, DMSO-d₆) δ 9.95 (br s, 2H), 8.57 (s, 2H), 8.49 (s, 1H), 8.19 (s, 1H), 8.17 (s, 1H), 7.88 (s, 1H), 3.89 (s, 3H), 3.63-3.66 (m, 4H), 3.16 (s, 2H), 2.52-2.53 (m, 4H), 2.40-2.43 (m, 3H).

### Example 57: 2-(1-methyl-1H-pyrazol-4-yl)-N-(2-methyl-5-(2-(1-methyl-6,7-dihydro-1H-pyrazolo[4,3-c]pyridin-5 (4H)-yl)acetamido)pyridin-3-yl)pyrazolo[5,1-b]thiazole-7-carboxamide

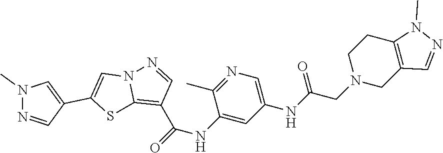 [SMILES: Cc1ncc(NC(=O)CN2CCc3c(cnn3C)C2)cc1NC(=O)c1cnn2cc(-c3cnn(C)c3)sc12]

To a mixture of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.20 mmol), K₂CO₃ (84 mg, 0.61 mmoL) and NaI (18 mg, 0.12 mmol) in DMF (2 mL) was added 1-methyl-4,5,6,7-tetrahydro-1H-pyrazolo[4,3-c]pyridine hydrochloride (42 mg, 0.24 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Xtimate C18 150*40 mm*5 um to give the title compound (2-(1-methyl-1H-pyrazol-4-yl)-N-(2-methyl-5-(2-(1-methyl-6,7-dihydro-1H-pyrazolo[4,3-c]pyridin-5 (4H)-yl)acetamido)pyridin-3-yl)pyrazolo[5,1-b]thiazole-7-carboxamide (60 mg, 56%) as a white solid. LCMS (ESI): mass calcd. for C₂₅H₂₆N₁₀O₂S, 530.6; m/z found, 531.3 [M+H]⁺. ¹H NMR (400 MHz, DMSO-d₆) δ 10.05 (s, 1H), 9.95 (s, 1H), 8.62-8.66 (m, 2H), 8.56 (s, 1H), 8.23-8.27 (m, 2H), 7.94 (s, 1H), 7.22 (s, 1H), 3.94 (s, 3H), 3.73 (s, 3H), 3.60-3.61 (m, 2H), 3.40-3.41 (m, 2H), 2.88-2.94 (m, 2H), 2.78-2.82 (m, 2H), 2.46 (s, 3H).

### Example 58: N-(5-(2-(6,7-dihydrothiazolo[5,4-c]pyridin-5 (4H)-yl)acetamido)-2-methylpyridin-3-yl)-2-(1-methyl-1H-pyrazol-4-yl)pyrazolo[5,1-b]thiazole-7-carboxamide

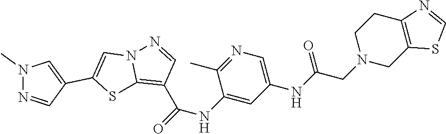 [SMILES: Cc1ncc(NC(=O)CN2CCc3ncsc3C2)cc1NC(=O)c1cnn2cc(-c3cnn(C)c3)sc12]

To a mixture of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.20 mmol), K₂CO₃ (84 mg, 0.61 mmoL) and NaI (18 mg, 0.12 mmol) in DMF (2 mL) was added 4,5,6,7-tetrahydrothiazolo[5,4-c]pyridine hydrochloride (43 mg, 0.24 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Phenomenex Gemini-NX C18 75*30 mm*3 um to give the title compound N-(5-(2-(6,7-dihydrothiazolo[5,4-c]pyridin-5 (4H)-yl)acetamido)-2-methylpyridin-3-yl)-2-(1-methyl-1H-pyrazol-4-yl)pyrazolo[5,1-b]thiazole-7-carboxamide (57.8 mg, 53%) as a white solid. LCMS (ESI): mass calcd. for C₂₄H₂₃N₉O₂S₂, 533.6; m/z found, 534.2 [M+H]⁺. ¹H NMR (400 MHz, DMSO-d₆) δ 10.07 (s, 1H), 9.89 (s, 1H), 8.93 (s, 1H), 8.55-8.60 (m, 1H), 8.51 (s, 1H), 8.19 (s, 1H), 8.19 (d, J=3.61 Hz, 2H), 7.88 (s, 1H), 3.86-3.91 (m, 5H), 3.41-3.43 (m, 2H), 2.96 (br d, J=5.36 Hz, 2H), 2.89 (br d, J=5.13 Hz, 2H), 2.41 (s, 3H).

### Example 59: N-(5-(2-(1,1-dioxidothiomorpholino)acetamido)-2-methylpyridin-3-yl)-2-(1-methyl-1H-pyrazol-4-yl)pyrazolo[5,1-b]thiazole-7-carboxamide

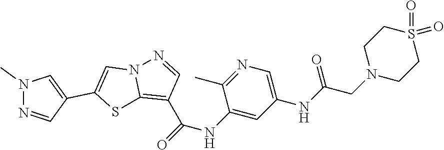 [SMILES: Cc1ncc(NC(=O)CN2CCS(=O)(=O)CC2)cc1NC(=O)c1cnn2cc(-c3cnn(C)c3)sc12]

To a mixture of N-(5-(2-chloroacetamido)-2-methylpyridin-3-yl)-2-(1-methyl-1H-pyrazol-4-yl)pyrazolo[5,1-b]thiazole-7-carboxamide (150 mg, 0.35 mmol), K₂CO₃ (145 mg, 1.0 mmoL) and NaI (31 mg, 0.21 mmol) in DMF (3 mL) was added thiomorpholine 1,1-dioxide (56.6 mg, 0.42 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Phenomenex Gemini-NX C18 75*30 mm*3 um to give the crude product and the crude product was purified by Supercritical Fluid Chromatography over column: DAICEL CHIRALPAK AS 250 mm*30 mm*10 um to give the title compound N-(5-(2-(1,1-dioxidothiomorpholino)acetamido)-2-methylpyridin-3-yl)-2-(1-methyl-1H-pyrazol-4-yl)pyrazolo[5,1-b]thiazole-7-carboxamide (54.5 mg, 30%) as a white solid. LCMS (ESI): mass calcd. for C₂₂H₂₄N₈O₄S₂, 528.6; m/z found, 529.3 [M+H]⁺. ¹H NMR (400 MHz, DMSO-d₆) δ 9.95 (s, 1H), 9.89 (s, 1H), 8.57-8.63 (m, 2H), 8.51 (s, 1H), 8.16-8.20 (m, 2H), 7.88 (s, 1H), 3.89 (s, 3H), 3.39-3.40 (m, 2H), 3.18-3.24 (m, 4H), 3.06-3.10 (m, 4H), 2.41-2.43 (m, 3H).

### Example 60: N-(5-(2-(5,6-dihydro-1,7-naphthyridin-7 (8H)-yl)acetamido)-2-methylpyridin-3-yl)-2-(1-methyl-1H-pyrazol-4-yl)pyrazolo[5,1-b]thiazole-7-carboxamide

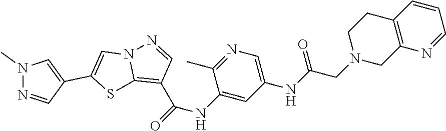 [SMILES: Cc1ncc(NC(=O)CN2CCc3cccnc3C2)cc1NC(=O)c1cnn2cc(-c3cnn(C)c3)sc12]

To a mixture of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.20 mmol), K₂CO₃ (84 mg, 0.61 mmoL) and NaI (18 mg, 0.12 mmol) in DMF (1 mL) was added 5,6,7,8-tetrahydro-1,7-naphthyridine hydrochloride (41.6 mg, 0.24 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Phenomenex Gemini-NX 80*40 mm*3 um to give the title compound N-(5-(2-(5,6-dihydro-1,7-naphthyridin-7 (8H)-yl)acetamido)-2-methylpyridin-3-yl)-2-(1-methyl-1H-pyrazol-4-yl)pyrazolo[5,1-b]thiazole-7-carboxamide (58.3 mg, 54%) as a white solid. LCMS (ESI): mass calcd. for C₂₆H₂₅N₉O₂S, 527.6; m/z found, 528.3 [M+H]⁺. ¹H NMR (400 MHz, DMSO-d₆) δ 10.06 (s, 1H), 9.89 (s, 1H), 8.55-8.62 (m, 2H), 8.51 (s, 1H), 8.33 (d, J=4.41 Hz, 1H), 8.17-8.22 (m, 2H), 7.88 (s, 1H), 7.56 (d, J=7.27 Hz, 1H), 7.19 (dd, J=7.63, 4.77 Hz, 1H), 3.88 (s, 3H), 3.79 (s, 2H), 3.40-3.41 (m, 2H), 2.82-2.93 (m, 4H), 2.38-2.42 (m, 3H).

### Example 61: N-(5-(2-(6,7-dihydrothiazolo[4,5-c]pyridin-5 (4H)-yl)acetamido)-2-methylpyridin-3-yl)-2-(1-methyl-1H-pyrazol-4-yl)pyrazolo[5,1-b]thiazole-7-carboxamide

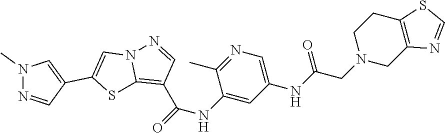 [SMILES: Cc1ncc(NC(=O)CN2CCc3scnc3C2)cc1NC(=O)c1cnn2cc(-c3cnn(C)c3)sc12]

To a mixture of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.20 mmol), K₂CO₃ (84 mg, 0.61 mmoL) and NaI (18 mg, 0.12 mmol) in DMF (1 mL) was added 4,5,6,7-tetrahydrothiazolo[4,5-c]pyridine (52 mg, 0.24 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Phenomenex Gemini-NX 80*40 mm*3 um to give the title compound N-(5-(2-(6,7-dihydrothiazolo[4,5-c]pyridin-5 (4H)-yl)acetamido)-2-methylpyridin-3-yl)-2-(1-methyl-1H-pyrazol-4-yl)pyrazolo[5,1-b]thiazole-7-carboxamide (50.2 mg, 46%) as a white solid. LCMS (ESI): mass calcd. for C₂₄H₂₃N₉O₂S, 533.6; m/z found, 534.2 [M+H]+. ¹H NMR (400 MHz, DMSO-d₆) δ 10.06 (s, 1H), 9.89 (s, 1H), 8.91 (s, 1H), 8.56-8.60 (m, 2H), 8.51 (s, 1H), 8.19 (d, J=3.64 Hz, 2H), 7.88 (s, 1H), 3.88 (s, 3H), 3.82 (s, 2H), 3.43-3.44 (m, 2H), 2.89-2.94 (m, 4H), 2.39-2.42 (m, 3H).

### Example 62: N-(5-(2-(3,4-dihydro-2,6-naphthyridin-2 (1H)-yl)acetamido)-2-methylpyridin-3-yl)-2-(1-methyl-1H-pyrazol-4-yl)pyrazolo[5,1-b]thiazole-7-carboxamide

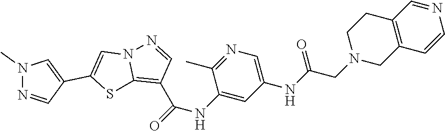 [SMILES: Cc1ncc(NC(=O)CN2CCc3cnccc3C2)cc1NC(=O)c1cnn2cc(-c3cnn(C)c3)sc12]

To a mixture of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.20 mmol), K₂CO₃ (84 mg, 0.61 mmoL) and NaI (18 mg, 0.12 mmol) in DMF (1 mL) was added 1,2,3,4-tetrahydro-2,6-naphthyridine hydrochloride (41.6 mg, 0.24 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Phenomenex Gemini-NX 80*40 mm*3 um to give the title compound N-(5-(2-(3,4-dihydro-2,6-naphthyridin-2 (1H)-yl)acetamido)-2-methylpyridin-3-yl)-2-(1-methyl-1H-pyrazol-4-yl)pyrazolo[5,1-b]thiazole-7-carboxamide (57.2 mg, 52%) as a white solid. LCMS (ESI): mass calcd. for C₂₆H₂₅N₉O₂S, 527.6; m/z found, 528.3 [M+H]⁺. ¹H NMR (400 MHz, DMSO-d₆) δ 9.77-10.22 (m, 2H), 8.54-8.60 (m, 2H), 8.49 (s, 1H), 8.36 (s, 1H), 8.28 (d, J=5.01 Hz, 1H), 8.19 (s, 2H), 7.88 (s, 1H), 7.12 (d, J=5.01 Hz, 1H), 3.88 (s, 3H), 3.77 (s, 2H), 3.38-3.39 (m, 2H), 2.86-2.92 (m, 4H), 2.39-2.42 (m, 3H).

### Example 63: N-(5-(2-(7,8-dihydro-1,6-naphthyridin-6 (5H)-yl)acetamido)-2-methylpyridin-3-yl)-2-(1-methyl-1H-pyrazol-4-yl)pyrazolo[5,1-b]thiazole-7-carboxamide

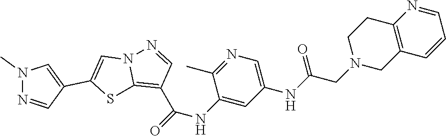 [SMILES: Cc1ncc(NC(=O)CN2CCc3ncccc3C2)cc1NC(=O)c1cnn2cc(-c3cnn(C)c3)sc12]

To a mixture of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.20 mmol), K₂CO₃ (84 mg, 0.61 mmoL) and NaI (18 mg, 0.12 mmol) in DMF (1 mL) was added 5,6,7,8-tetrahydro-1,6-naphthyridine hydrochloride (41.6 mg, 0.24 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Phenomenex Gemini-NX 80*40 mm*3 um to give the title compound N-(5-(2-(7,8-dihydro-1,6-naphthyridin-6 (5H)-yl)acetamido)-2-methylpyridin-3-yl)-2-(1-methyl-1H-pyrazol-4-yl)pyrazolo[5,1-b]thiazole-7-carboxamide (68.3 mg, 64%) as a white solid. LCMS (ESI): mass calcd. for C₂₆H₂₅N₉O₂S, 527.6; m/z found, 528.3 [M+H]⁺. ¹H NMR (400 MHz, DMSO-d₆) δ 9.81-10.11 (m, 2H), 8.53-8.61 (m, 2H), 8.49 (s, 1H), 8.35 (d, J=3.81 Hz, 1H), 8.19 (s, 2H), 7.88 (s, 1H), 7.50 (d, J=7.27 Hz, 1H), 7.16 (dd, J=7.69, 4.71 Hz, 1H), 3.87-3.90 (m, 3H), 3.78 (s, 2H), 3.39 (br s, 2H), 2.96 (br dd, J=14.72, 4.83 Hz, 4H), 2.39-2.43 (m, 3H).

### Example 64: (S)-2-(1-methyl-1H-pyrazol-4-yl)-N-(2-methyl-5-(2-(2-methylmorpholino)acetamido)pyridin-3-yl)pyrazolo[5,1-b]thiazole-7-carboxamide

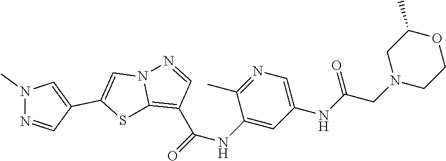 [SMILES: Cc1ncc(NC(=O)CN2CCO[C@@H](C)C2)cc1NC(=O)c1cnn2cc(-c3cnn(C)c3)sc12]

To a mixture of N-(5-(2-chloroacetamido)-2-methylpyridin-3-yl)-2-(1-methyl-1H-pyrazol-4-yl)pyrazolo[5,1-b]thiazole-7-carboxamide (70 mg, 0.16 mmol), K₂CO₃ (65 mg, 0.47 mmoL) and NaI (14 mg, 0.10 mmol) in DMF (1 mL) was added (S)-2-methylmorpholine (24 mg, 0.24 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Boston Prime C18 150*30 mm*5 um to give the title compound (S)-2-(1-methyl-1H-pyrazol-4-yl)-N-(2-methyl-5-(2-(2-methylmorpholino)acetamido)pyridin-3-yl)pyrazolo[5,1-b]thiazole-7-carboxamide (37.2 mg, 46%) as a white solid. LCMS (ESI): mass calcd. for C₂₃H₂₆N₈O₃S, 494.6; m/z found, 495.2 [M+H]⁺. ¹H NMR (400 MHz, DMSO-d₆) δ ppm 9.97 (s, 1H), 9.90 (s, 1H), 8.59 (s, 2H), 8.51 (s, 1H), 8.15-8.21 (m, 2H), 7.89 (s, 1H), 3.89 (s, 3H), 3.75 (dd, J=11.15, 1.73 Hz, 1H), 3.58-3.65 (m, 2H), 3.14-3.17 (m, 2H), 2.70-2.80 (m, 2H), 2.40-2.42 (m, 3H), 2.20-2.27 (m, 1H), 1.93 (t, J=10.49 Hz, 1H), 1.05 (d, J=6.32 Hz, 3H).

### Example 65: (S)-2-(1-methyl-1H (R)—N-(5-(2-(2-ethylmorpholino)acetamido)-2-methylpyridin-3-yl)-2-(1-methyl-1H-pyrazol-4-yl)pyrazolo[5,1-b]thiazole-7-carboxamide

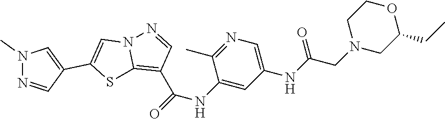 [SMILES: CC[C@@H]1CN(CC(=O)Nc2cnc(C)c(NC(=O)c3cnn4cc(-c5cnn(C)c5)sc34)c2)CCO1]

To a mixture of N-(5-(2-chloroacetamido)-2-methylpyridin-3-yl)-2-(1-methyl-1H-pyrazol-4-yl)pyrazolo[5,1-b]thiazole-7-carboxamide (150 mg, 0.35 mmol), K₂CO₃ (144 mg, 1.0 mmoL) and NaI (31 mg, 0.21 mmol) in DMF (5 mL) was added 2-ethylmorpholine (48 mg, 0.42 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Boston Prime C18 150*30 mm*5 um to give the crude product and the crude product was purified by Supercritical Fluid Chromatography over column: DAICEL CHIRALPAK IG 250 mm*30 mm*10 um to give title compound (R)—N-(5-(2-(2-ethylmorpholino)acetamido)-2-methylpyridin-3-yl)-2-(1-methyl-1H-pyrazol-4-yl)pyrazolo[5,1-b]thiazole-7-carboxamide (65.2 mg, 43%) as a white solid. LCMS (ESI): mass calcd. for C₂₄H₂₈N₈O₃S, 508.6; m/z found, 509.3 [M+H]⁺. ¹H NMR (400 MHz, DMSO-d₆) δ 9.96 (s, 1H), 9.89 (s, 1H), 8.59 (s, 2H), 8.51 (s, 1H), 8.20 (s, 1H), 8.16 (s, 1H), 7.89 (s, 1H), 3.89 (s, 3H), 3.75-3.81 (m, 1H), 3.57-3.64 (m, 1H), 3.40-3.43 (m, 1H), 3.16 (s, 2H), 2.80 (br d, J=11.21 Hz, 1H), 2.69-2.73 (m, 1H), 2.40-2.43 (m, 3H), 2.22-2.28 (m, 1H), 1.96 (t, J=10.49 Hz, 1H), 1.36-1.44 (m, 2H), 0.88 (t, J=7.51 Hz, 3H).

### Example 66: (S)—N-(5-(2-(2-ethylmorpholino)acetamido)-2-methylpyridin-3-yl)-2-(1-methyl-1H-pyrazol-4-yl)pyrazolo[5,1-b]thiazole-7-carboxamide

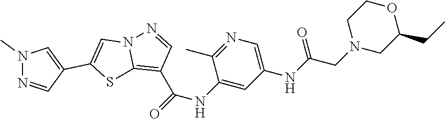 [SMILES: CC[C@H]1CN(CC(=O)Nc2cnc(C)c(NC(=O)c3cnn4cc(-c5cnn(C)c5)sc34)c2)CCO1]

To a mixture of N-(5-(2-chloroacetamido)-2-methylpyridin-3-yl)-2-(1-methyl-1H-pyrazol-4-yl)pyrazolo[5,1-b]thiazole-7-carboxamide (150 mg, 0.35 mmol), K₂CO₃ (144 mg, 1.0 mmoL) and NaI (31 mg, 0.21 mmol) in DMF (5 mL) was added 2-ethylmorpholine (48 mg, 0.42 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Boston Prime C18 150*30 mm*5 um to give the crude product and the crude product was purified by Supercritical Fluid Chromatography over column: DAICEL CHIRALPAK IG 250 mm*30 mm*10 um to give title compound (S)—N-(5-(2-(2-ethylmorpholino)acetamido)-2-methylpyridin-3-yl)-2-(1-methyl-1H-pyrazol-4-yl)pyrazolo[5,1-b]thiazole-7-carboxamide (52.8 mg, 35%) as a white solid. LCMS (ESI): mass calcd. for C₂₄H₂₈N₈O₃S, 508.6; m/z found, 509.3 [M+H]⁺. ¹H NMR (400 MHz, DMSO-d₆) δ ppm 9.96 (s, 1H), 9.89 (s, 1H), 8.59 (s, 2H), 8.51 (s, 1H), 8.20 (s, 1H), 8.16 (s, 1H), 7.88 (s, 1H), 3.89 (s, 3H), 3.78 (dd, J=11.32, 1.67 Hz, 1H), 3.56-3.64 (m, 1H), 3.39-3.44 (m, 1H), 3.16 (s, 2H), 2.80 (br d, J=10.85 Hz, 1H), 2.69-2.75 (m, 1H), 2.39-2.42 (m, 3H), 2.21-2.28 (m, 1H), 1.96 (t, J=10.55 Hz, 1H), 1.35-1.43 (m, 2H), 0.88 (t, J=7.51 Hz, 3H).

### Example 67: N-(5-(2-(2,2-dimethylmorpholino)acetamido)-2-methylpyridin-3-yl)-2-(1-methyl-1H-pyrazol-4-yl)pyrazolo[5,1-b]thiazole-7-carboxamide

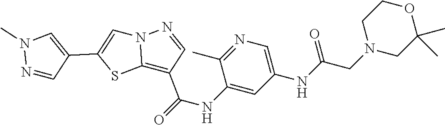

To a mixture of N-(5-(2-chloroacetamido)-2-methylpyridin-3-yl)-2-(1-methyl-1H-pyrazol-4-yl)pyrazolo[5,1-b]thiazole-7-carboxamide (83 mg, 0.18 mmol), K₂CO₃ (75 mg, 0.55 mmoL) and NaI (16 mg, 0.11 mmol) in DMF (3 mL) was added 2,2-dimethylmorpholine (25.1 mg, 0.22 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Phenomenex Synergi C18 150*30 mm*4 um to give title compound N-(5-(2-(2,2-dimethylmorpholino)acetamido)-2-methylpyridin-3-yl)-2-(1-methyl-1H-pyrazol-4-yl)pyrazolo[5,1-b]thiazole-7-carboxamide (Formic acid salt, 32.3 mg, 34%) as a white solid. LCMS (ESI): mass calcd. for C₂₄H₂₈N₈O₃S, 508.6; m/z found, 509.1 [M+H]⁺. ¹H NMR (400 MHz, METHANOL-d₄) δ 8.58-8.61 (m, 1H), 8.60 (d, J=2.38 Hz, 1H), 8.27 (d, J=2.26 Hz, 1H), 8.22 (s, 1H), 8.03 (s, 1H), 7.83 (s, 1H), 3.94-3.97 (m, 1H), 3.95 (s, 2H), 3.81-3.86 (m, 2H), 3.18 (s, 2H), 2.55-2.60 (m, 2H), 2.52 (s, 3H), 2.40 (s, 2H), 1.32 (s, 6H).

### Example 68: N-(5-(2-(3,3-dimethylmorpholino)acetamido)-2-methylpyridin-3-yl)-2-(1-methyl-1H-pyrazol-4-yl)pyrazolo[5,1-b]thiazole-7-carboxamide

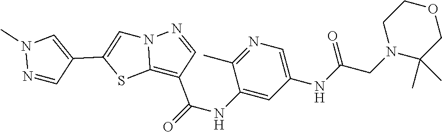

To a mixture of N-(5-(2-chloroacetamido)-2-methylpyridin-3-yl)-2-(1-methyl-1H-pyrazol-4-yl)pyrazolo[5,1-b]thiazole-7-carboxamide (80 mg, 0.17 mmol), K₂CO₃ (70 mg, 0.51 mmoL) and NaI (15 mg, 0.10 mmol) in DMF (3 mL) was added 3,3-dimethylmorpholine (23 mg, 0.20 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Phenomenex Synergi C18 150*30 mm*4 um to give title compound N-(5-(2-(3,3-dimethylmorpholino)acetamido)-2-methylpyridin-3-yl)-2-(1-methyl-1H-pyrazol-4-yl)pyrazolo[5,1-b]thiazole-7-carboxamide (Formic acid salt, 32.5 mg, 38%) as a white solid. LCMS (ESI): mass calcd. for C₂₄H₂₈N₈O₃S, 508.6; m/z found, 509.4 [M+H]⁺. ¹H NMR (400 MHz, DMSO-d₆) δ 9.90 (s, 1H), 9.79 (s, 1H), 8.63 (d, J=1.91 Hz, 1H), 8.57 (s, 1H), 8.50 (s, 1H), 8.19 (s, 1H), 8.15 (d, J=1.91 Hz, 1H), 7.88 (s, 1H), 3.88 (s, 3H), 3.66-3.73 (m, 2H), 2.94-3.26 (m, 2H), 2.54 (br d, J=4.05 Hz, 4H), 2.40 (s, 3H), 1.00 (s, 6H).

### Example 69: (R)—N-(5-(2-(3-ethylmorpholino)acetamido)-2-methylpyridin-3-yl)-2-(1-methyl-1H-pyrazol-4-yl)pyrazolo[5,1-b]thiazole-7-carboxamide

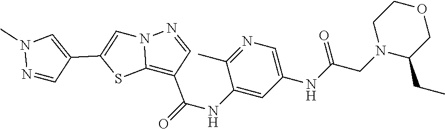 [SMILES: CC[C@@H]1COCCN1CC(=O)Nc1cnc(C)c(NC(=O)c2cnn3cc(-c4cnn(C)c4)sc23)c1]

To a mixture of N-(5-(2-chloroacetamido)-2-methylpyridin-3-yl)-2-(1-methyl-1H-pyrazol-4-yl)pyrazolo[5,1-b]thiazole-7-carboxamide (80 mg, 0.17 mmol), K₂CO₃ (70 mg, 0.51 mmoL) and NaI (15 mg, 0.10 mmol) in DMF (3 mL) was added (R)-3-ethylmorpholine (23 mg, 0.20 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Phenomenex Synergi C18 150*30 mm*4 um to give title compound (R)—N-(5-(2-(3-ethylmorpholino)acetamido)-2-methylpyridin-3-yl)-2-(1-methyl-1H-pyrazol-4-yl)pyrazolo[5,1-b]thiazole-7-carboxamide (20 mg, 23%) as a white solid. LCMS (ESI): mass calcd. for C₂₄H₂₈N₈O₃S, 508.6; m/z found, 509.1 [M+H]⁺. ¹H NMR (400 MHz, METHANOL-d₄) δ 8.64 (d, J=2.03 Hz, 1H), 8.41 (s, 1H), 8.23-8.31 (m, 2H), 8.05 (s, 1H), 7.85 (s, 1H), 3.96 (s, 3H), 3.87 (br dd, J=11.44, 2.38 Hz, 1H), 3.75-3.83 (m, 2H), 3.46-3.55 (m, 2H), 3.16 (br d, J=16.69 Hz, 1H), 2.88 (br d, J=11.92 Hz, 1H), 2.56-2.65 (m, 1H), 2.52 (s, 3H), 2.48 (br d, J=9.06 Hz, 1H), 1.60-1.77 (m, 1H), 1.44-1.56 (m, 1H), 0.94 (t, J=7.51 Hz, 2H), 0.90-0.99 (m, 1H).

### Example 70: (S)—N-(5-(2-(3-ethylmorpholino)acetamido)-2-methylpyridin-3-yl)-2-(1-methyl-1H-pyrazol-4-yl)pyrazolo[5,1-b]thiazole-7-carboxamide

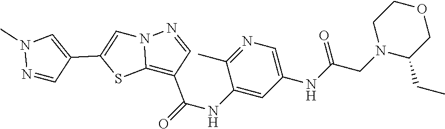 [SMILES: CC[C@H]1COCCN1CC(=O)Nc1cnc(C)c(NC(=O)c2cnn3cc(-c4cnn(C)c4)sc23)c1]

To a mixture of N-(5-(2-chloroacetamido)-2-methylpyridin-3-yl)-2-(1-methyl-1H-pyrazol-4-yl)pyrazolo[5,1-b]thiazole-7-carboxamide (80 mg, 0.14 mmol), K₂CO₃ (58 mg, 0.42 mmoL) and NaI (12 mg, 0.08 mmol) in DMSO (3 mL) was added (S)-3-ethylmorpholine (19 mg, 0.17 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Phenomenex Synergi C18 150*30 mm*4 um to give title compound (S)—N-(5-(2-(3-ethylmorpholino)acetamido)-2-methylpyridin-3-yl)-2-(1-methyl-1H-pyrazol-4-yl)pyrazolo[5,1-b]thiazole-7-carboxamide (48.5 mg, 66%) as a white solid. LCMS (ESI): mass calcd. for C₂₄H₂₈N₈O₃S, 508.6; m/z found, 509.4 [M+H]⁺. ¹H NMR (400 MHz, METHANOL-d₄) δ 8.60-8.68 (m, 1H), 8.41 (s, 1H), 8.21-8.30 (m, 2H), 8.00-8.09 (m, 1H), 8.00-8.09 (m, 1H), 3.96 (s, 3H), 3.87 (dd, J=11.32, 2.98 Hz, 1H), 3.72-3.83 (m, 2H), 3.43-3.56 (m, 2H), 3.16 (d, J=16.69 Hz, 1H), 2.87 (dt, J=11.80, 2.98 Hz, 1H), 2.60 (ddd, J=11.92, 8.52, 3.76 Hz, 1H), 2.52 (s, 3H), 2.47 (br t, J=8.64 Hz, 1H), 1.59-1.71 (m, 1H), 1.42-1.55 (m, 1H), 0.88-1.00 (m, 3H).

### Example 71: N-(5-(2-((3S,5R)-3,5-dimethylmorpholino)acetamido)-2-methylpyridin-3-yl)-2-(1-methyl-1H-pyrazol-4-yl)pyrazolo[5,1-b]thiazole-7-carboxamide

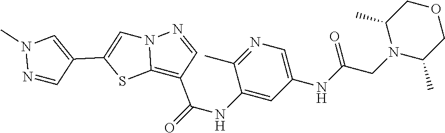 [SMILES: Cc1ncc(NC(=O)CN2[C@H](C)COC[C@@H]2C)cc1NC(=O)c1cnn2cc(-c3cnn(C)c3)sc12]

To a mixture of N-(5-(2-chloroacetamido)-2-methylpyridin-3-yl)-2-(1-methyl-1H-pyrazol-4-yl)pyrazolo[5,1-b]thiazole-7-carboxamide (80 mg, 0.19 mmol), K₂CO₃ (77 mg, 0.56 mmoL) and NaI (17 mg, 0.11 mmol) in DMF (3 mL) was added (3S,5R)-3,5-dimethylmorpholine (26 mg, 0.22 mmol). The reaction mixture was stirred at 50° C. for 2 h before cooling to room temperature. The reaction mixture was filtered and the filtrate was concentrated under vacuum to give crude product, which was purified by preparative high-performance liquid chromatography over column: Phenomenex Synergi C18 150*30 mm*4 um to give title compound N-(5-(2-((3S,5R)-3,5-dimethylmorpholino)acetamido)-2-methylpyridin-3-yl)-2-(1-methyl-1H-pyrazol-4-yl)pyrazolo[5,1-b]thiazole-7-carboxamide (Formic acid salt, 31.1 mg, 32%) as a white solid. LCMS (ESI): mass calcd. for C₂₄H₂₈N₈O₃S, 508.6; m/z found, 509.2 [M+H]⁺. ¹H NMR (400 MHz, DMSO-d₆) δ 9.89-9.96 (m, 1H), 9.84 (s, 1H), 8.60-8.64 (m, 1H), 8.58 (s, 1H), 8.52 (s, 1H), 8.30 (s, 1H), 8.20 (s, 1H), 8.15 (d, J=2.15 Hz, 1H), 7.88 (s, 1H), 3.89 (s, 3 H), 3.64 (br dd, J=10.97, 2.86 Hz, 1H), 3.34 (br s, 1H), 3.24 (br s, 1H), 3.21 (br s, 1H), 3.15-3.20 (m, 2H), 2.66-2.79 (m, 1H), 2.41 (s, 3H), 0.84-1.04 (m, 6H).

### Example 72: 2-(1-methyl-1H-pyrazol-4-yl)-N-(2-methyl-5-(2-(4-methyl-3-oxopiperazin-1-yl)acetamido)pyridin-3-yl)pyrazolo[5,1-b]thiazole-7-carboxamide

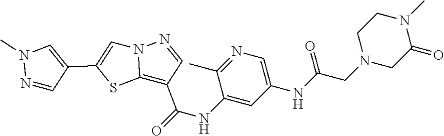 [SMILES: Cc1ncc(NC(=O)CN2CCN(C)C(=O)C2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.19 mmol), 1-methylpiperazin-2-one (26 mg, 0.22 mmol) in DMF (3 mL) was added K₂CO₃ (77 mg, 0.56 mmol) and NaI (17 mg, 0.11 mmol). The resulting mixture was stirred at 50° C. for 2 hours. The reaction mixture was filtered over a pad of celite and concentrated under vacuum to give crude as black solid, which was purified by preparative high-performance liquid chromatography over column: Welch Xtimate C18 150*30 mm*5 um to give the title compound 2-(1-methyl-1H-pyrazol-4-yl)-N-(2-methyl-5-(2-(4-methyl-3-oxopiperazin-1-yl)acetamido)pyridin-3-yl)pyrazolo[5,1-b]thiazole-7-carboxamide (20 mg, 21%) as a white solid. LCMS (ESI): mass calcd. for C₂₃H₂₅N₉O₃S, 507.2; m/z found, 508.3 [M+H]⁺. ¹H NMR (400 MHz, CDCl₃) δ 8.84 (br s, 1H), 8.71 (br s, 1H), 8.49 (br s, 1H), 8.10 (br s, 1H), 7.83 (br s, 1H), 7.70 (br s, 1H), 7.63 (br s, 1H), 7.54 (br s, 1H), 3.98 (br s, 3H), 3.45 (br s, 2H), 3.34 (br s, 2H), 3.26 (br s, 2H), 3.03 (br s, 3H), 2.90 (br s, 2H), 2.59 (br s, 3H).

### Example 73: N-(5-(2-(3,3-difluoropyrrolidin-1-yl)acetamido)-2-methylpyridin-3-yl)-2-(1-methyl-1H-pyrazol-4-yl)pyrazolo[5,1-b]thiazole-7-carboxamide

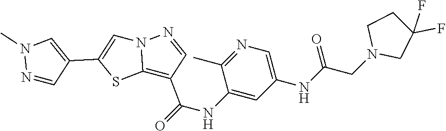 [SMILES: Cc1ncc(NC(=O)CN2CCC(F)(F)C2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.19 mmol), 3,3-difluoropyrrolidine (24 mg, 0.22 mmol) in DMF (3 mL) was added K₂CO₃ (77 mg, 0.56 mmol) and NaI (17 mg, 0.11 mmol). The resulting mixture was stirred at 50° C. for 2 hours. The reaction mixture was filtered over a pad of celite and concentrated under vacuum to give crude as black solid, which was purified by preparative high-performance liquid chromatography over column: Welch Xtimate C18 150*30 mm*5 um to give the title compound N-(5-(2-(3,3-difluoropyrrolidin-1-yl)acetamido)-2-methylpyridin-3-yl)-2-(1-methyl-1H-pyrazol-4-yl)pyrazolo[5,1-b]thiazole-7-carboxamide (19 mg, 20%) as a white solid. LCMS (ESI): mass calcd. for C₂₂H₂₂F₂N₈O₂S, 500.2; m/z found, 501.3 [M+H]⁺. ¹H NMR (400 MHz, CDCl₃) δ 8.85 (br s, 1H), 8.69 (br s, 1H), 8.53 (br s, 1H), 8.07 (br d, J=4.0 Hz, 1H), 7.82 (br d, J=4.6 Hz, 1H), 7.70 (br d, J=4.4 Hz, 1H), 7.62 (br d, J=4.6 Hz, 1H), 7.43 (br s, 1H), 3.98 (br d, J=4.6 Hz, 3H), 3.35 (br d, J=4.2 Hz, 2H), 3.17-3.05 (m, 2H), 2.98 (br d, J=4.9 Hz, 2H), 2.58 (br d, J=4.4 Hz, 3H), 2.50-2.35 (m, 2H).

### Example 74: (S)-2-(1-methyl-1H-pyrazol-4-yl)-N-(2-methyl-5-(2-(3-methylmorpholino)acetamido)pyridin-3-yl)pyrazolo[5,1-b]thiazole-7-carboxamide

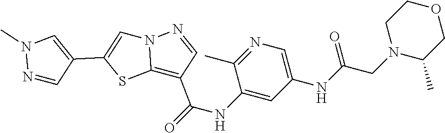 [SMILES: Cc1ncc(NC(=O)CN2CCOC[C@@H]2C)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (80 mg, 0.18 mmol), (S)-3-methylmorpholine (22 mg, 0.22 mmol) in DMF (2 mL) was added K₂CO₃ (75 mg, 0.54 mmol) and NaI (16 mg, 0.11 mmol). The resulting mixture was stirred at 50° C. for 2 hours. The reaction mixture was filtered over a pad of celite and concentrated under vacuum to give crude as black solid, which was purified by preparative high-performance liquid chromatography over column: Welch Xtimate C18 150*30 mm*5 um to give the title compound (S)-2-(1-methyl-1H-pyrazol-4-yl)-N-(2-methyl-5-(2-(3-methylmorpholino)acetamido)pyridin-3-yl)pyrazolo[5,1-b]thiazole-7-carboxamide (43 mg, 46%) as a white solid. LCMS (ESI): mass calcd. for C₂₃H₂₆N₈O₃S, 494.2; m/z found, 495.2 [M+H]⁺. ¹H NMR (400 MHz, CDCl₃) δ 9.15 (s, 1H), 8.68 (d, J=1.8 Hz, 1H), 8.50 (d, J=1.7 Hz, 1H), 8.08 (s, 1H), 7.82 (s, 1H), 7.70 (s, 1H), 7.61 (s, 1H), 7.44 (s, 1H), 3.98 (s, 3H), 3.90-3.82 (m, 1H), 3.81-3.69 (m, 2H), 3.45 (d, J=17.0 Hz, 1H), 3.40-3.30 (m, 1H), 3.00 (d, J=17.0 Hz, 1H), 2.80 (br d, J=11.7 Hz, 1H), 2.62 (br s, 2H), 2.57 (s, 3H), 1.03 (d, J=6.3 Hz, 3H).

### Example 75: N-(5-(2-(5,6-dihydroimidazo[1,2-a]pyrazin-7 (8H)-yl)acetamido)-2-methylpyridin-3-yl)-2-(1-methyl-1H-pyrazol-4-yl)pyrazolo[5,1-b]thiazole-7-carboxamide

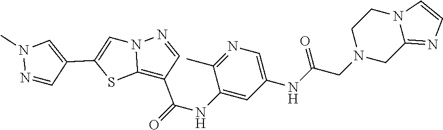 [SMILES: Cc1ncc(NC(=O)CN2CCn3ccnc3C2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.20 mmol), 5,6,7,8-tetrahydroimidazo[1,2-a]pyrazine (30 mg, 0.24 mmol) in DMF (2 mL) was added K₂CO₃ (84 mg, 0.61 mmol) and NaI (18 mg, 0.12 mmol). The resulting mixture was stirred at 50° C. for 2 hours. The reaction mixture was filtered over a pad of celite and concentrated under vacuum to give crude as black solid, which was purified by preparative high-performance liquid chromatography over column: Phenomenex Gemini-NX 80*40 mm*3 um to give the title compound N-(5-(2-(5,6-dihydroimidazo[1,2-a]pyrazin-7 (8H)-yl)acetamido)-2-methylpyridin-3-yl)-2-(1-methyl-1H-pyrazol-4-yl)pyrazolo[5,1-b]thiazole-7-carboxamide (49 mg, 46%) as a white solid. LCMS (ESI): mass calcd. for C₂₄H₂₄N₁₀O₂S, 516.2; m/z found, 517.3 [M+H]⁺. ¹H NMR (400 MHz, CDCl₃) δ 8.94 (s, 1H), 8.66 (d, J=2.0 Hz, 1H), 8.49 (d, J=2.2 Hz, 1H), 8.08 (s, 1H), 7.81 (s, 1H), 7.69 (s, 1H), 7.61 (s, 1H), 7.57 (s, 1H), 7.05 (s, 1H), 6.90 (s, 1H), 4.13 (br t, J=5.4 Hz, 2H), 3.97 (s, 3H), 3.93 (s, 2H), 3.40 (s, 2H), 3.09 (br t, J=5.4 Hz, 2H), 2.57 (s, 3H).

### Example 76: N-(5-(2-(4H-pyrrolo[3,4-d]thiazol-5 (6H)-yl)acetamido)-2-methylpyridin-3-yl)-2-(1-methyl-1H-pyrazol-4-yl)pyrazolo[5,1-b]thiazole-7-carboxamide

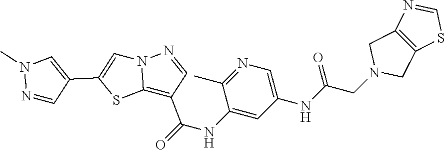 [SMILES: Cc1ncc(NC(=O)CN2Cc3ncsc3C2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.20 mmol), 5,6-dihydro-4H-pyrrolo[3,4-d]thiazole hydrobromide (50 mg, 0.24 mmol) in DMF (2 mL) was added K₂CO₃ (84 mg, 0.61 mmol) and NaI (18 mg, 0.12 mmol). The resulting mixture was stirred at 50° C. for 2 hours. The reaction mixture was filtered over a pad of celite and concentrated under vacuum to give crude as black solid, which was purified by preparative high-performance liquid chromatography over column: Phenomenex Gemini-NX 80*40 mm*3 um to give the title compound N-(5-(2-(4H-pyrrolo[3,4-d]thiazol-5 (6H)-yl)acetamido)-2-methylpyridin-3-yl)-2-(1-methyl-1H-pyrazol-4-yl)pyrazolo[5,1-b]thiazole-7-carboxamide (59 mg, 54%) as a white solid. LCMS (ESI): mass calcd. for C₂₃H₂₁N₉O₂S₂, 519.1; m/z found, 520.2 [M+H]⁺. ¹H NMR (400 MHz, DMSO-d₆) δ 10.12 (br s, 1H), 9.91 (br s, 1H), 9.02 (s, 1H), 8.58-8.55 (m, 2H), 8.48 (br s, 1H), 8.22 (d, J=1.9 Hz, 1H), 8.19 (s, 1H), 7.87 (s, 1H), 4.21 (br d, J=3.3 Hz, 2H), 4.10 (d, J=3.6 Hz, 2H), 3.88 (s, 3H), 3.69 (s, 2H), 2.41 (s, 3H).

### Example 77: N-(5-(2-(1H-pyrrolo[3,4-c]pyridin-2 (3H)-yl)acetamido)-2-methylpyridin-3-yl)-2-(1-methyl-1H-pyrazol-4-yl)pyrazolo[5,1-b]thiazole-7-carboxamide

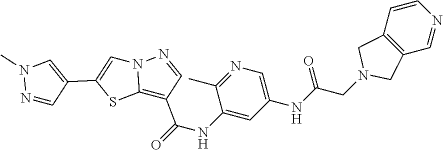 [SMILES: Cc1ncc(NC(=O)CN2Cc3ccncc3C2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.20 mmol), 2,3-dihydro-1H-pyrrolo[3,4-c]pyridine hydrochloride (47 mg, 0.24 mmol) in DMF (2 mL) was added K₂CO₃ (84 mg, 0.61 mmol) and NaI (18 mg, 0.12 mmol). The resulting mixture was stirred at 50° C. for 2 hours. The reaction mixture was filtered over a pad of celite and concentrated under vacuum to give crude as black solid, which was purified by preparative high-performance liquid chromatography over column: Phenomenex Gemini-NX 80*40 mm*3 um to give the title compound N-(5-(2-(1H-pyrrolo[3,4-c]pyridin-2 (3H)-yl)acetamido)-2-methylpyridin-3-yl)-2-(1-methyl-1H-pyrazol-4-yl)pyrazolo[5,1-b]thiazole-7-carboxamide (62 mg, 59%) as a white solid. LCMS (ESI): mass calcd. for C₂₅H₂₃N₉O₂S, 513.2; m/z found, 514.2 [M+H]⁺. ¹H NMR (400 MHz, DMSO-d₆) δ 10.23-9.79 (m, 2H), 8.59-8.53 (m, 2H), 8.51-8.45 (m, 2H), 8.41 (d, J=4.9 Hz, 1H), 8.21-8.16 (m, 2H), 7.87 (s, 1H), 7.33 (d, J=4.9 Hz, 1H), 4.13 (br s, 2H), 4.11 (br s, 2H), 3.88 (s, 3H), 3.62 (s, 2H), 2.40 (s, 3H).

### Example 78: N-(5-(2-(8-oxa-5-azaspiro[3.5]nonan-5-yl)acetamido)-2-methylpyridin-3-yl)-2-(1-methyl-1H-pyrazol-4-yl)pyrazolo[5,1-b]thiazole-7-carboxamide

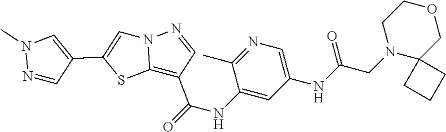 [SMILES: Cc1ncc(NC(=O)CN2CCOCC23CCC3)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (80 mg, 0.17 mmol), 8-oxa-5-azaspiro[3.5]nonane (26 mg, 0.20 mmol) in DMF (2 mL) was added K₂CO₃ (70 mg, 0.51 mmol) and NaI (15 mg, 0.10 mmol). The resulting mixture was stirred at 50° C. for 2 hours. The reaction mixture was filtered over a pad of celite and concentrated under vacuum to give crude as brown solid, which was purified by preparative high-performance liquid chromatography over column: Phenomenex Gemini-NX 80*40 mm*3 um to give the title compound N-(5-(2-(8-oxa-5-azaspiro[3.5]nonan-5-yl)acetamido)-2-methylpyridin-3-yl)-2-(1-methyl-1H-pyrazol-4-yl)pyrazolo[5,1-b]thiazole-7-carboxamide (26 mg, 29%) as a white solid. LCMS (ESI): mass calcd. for C₂₅H₂₈N₈O₃S, 520.2; m/z found, 521.3 [M+H]⁺. ¹H NMR (400 MHz, CDCl₃) δ 9.18 (s, 1H), 8.58 (d, J=2.3 Hz, 1H), 8.44 (d, J=2.1 Hz, 1H), 8.00 (s, 1H), 7.75 (s, 1H), 7.63 (s, 1H), 7.55 (s, 1H), 7.34 (s, 1H), 3.91 (s, 3H), 3.74-3.67 (m, 2H), 3.62 (s, 2H), 3.28 (s, 2H), 2.61-2.54 (m, 2H), 2.51 (s, 3H), 2.07-1.97 (m, 2H), 1.86-1.75 (m, 2H), 1.74-1.63 (m, 2H).

### Example 79: N-(5-(2-(2-(methoxymethyl)pyrrolidin-1-yl)acetamido)-2-methylpyridin-3-yl)-2-(1-methyl-1H-pyrazol-4-yl)pyrazolo[5,1-b]thiazole-7-carboxamide

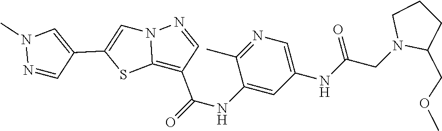 [SMILES: COCC1CCCN1CC(=O)Nc1cnc(C)c(NC(=O)c2cnn3cc(-c4cnn(C)c4)sc23)c1]

To a mixture of (6-methyl-5-(2-(1-methyl-1H-pyrazol-4-yl)pyrazolo[5,1-b]thiazole-7-carboxamido)pyridin-3-yl)carbamic chloride (111 mg, 0.24 mmol) in DMF (4 mL) was added 2-(methoxymethyl)pyrrolidine (40 mg, 0.35 mmol) and K₂CO₃ (96 mg, 0.70 mmol), then NaI (21 mg, 0.14 mmol) was added. LCMS showed 41.4% desired MS was detected. The reaction mixture was concentrated under vacuum to give the crude product, which was purified by preparative high-performance liquid chromatography over column: Phenomenex Gemini-NX 80*40 mm*3 um. The pure fractions were collected and the solvent was evaporated under vacuum, lyophilized to dryness to give N-(5-(2-(2-(methoxymethyl)pyrrolidin-1-yl)acetamido)-2-methylpyridin-3-yl)-2-(1-methyl-1H-pyrazol-4-yl)pyrazolo[5,1-b]thiazole-7-carboxamide (38.7 mg, 99%) as white solid. LCMS (ESI): mass for C₂₄H₂₈N₈O₃S: 508.6; m/z found: 509.4 [M+H]⁺. ¹H NMR (400 MHz, DMSO-d₆) δ 9.99-9.77 (m, 2H), 8.62-8.53 (m, 2H), 8.50 (s, 1H), 8.21-8.12 (m, 2H), 7.87 (s, 1H), 3.87 (s, 3H), 3.52 (d, J=16.3 Hz, 1H), 3.35 (br d, J=6.2 Hz, 1H), 3.30-3.24 (m, 2H), 3.22-3.20 (m, 3H), 3.12-3.04 (m, 1H), 2.82 (td, J=6.4, 13.1 Hz, 1H), 2.48-2.43 (m, 1H), 2.40 (s, 3H), 1.94-1.81 (m, 1H), 1.80-1.63 (m, 2H), 1.49 (tdd, J=6.0, 8.7, 12.1 Hz, 1H).

### Example 80: N-(5-(2-(1,4-oxazepan-4-yl)acetamido)-2-methylpyridin-3-yl)-2-(1-methyl-1H-pyrazol-4-yl)pyrazolo[5,1-b]thiazole-7-carboxamide

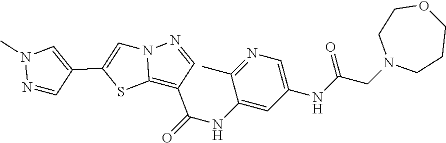 [SMILES: Cc1ncc(NC(=O)CN2CCCOCC2)cc1NC(=O)c1cnn2cc(-c3cnn(C)c3)sc12]

To the mixture of N-(5-(2-chloroacetamido)-2-methylpyridin-3-yl)-2-(1-methyl-1H-pyrazol-4-yl)pyrazolo[5,1-b]thiazole-7-carboxamide (120 mg, 0.23 mmol) in DMF (2 mL) was added 1,4-oxazepane hydrochloride (38 mg, 0.28 mmol), K₂CO₃ (97 mg, 0.70 mmol), NaI (21 mg, 0.14 mmol). The reaction mixture was stirred at 50° C. for 2 hours. LCMS showed desired MS was detected. The mixture was concentrated under vacuo to remove the solvent and purified by preparative high-performance liquid chromatography over column: Boston Prime C18 150*30 mm*5 um. The pure fractions were collected and the solvent was evaporated under vacuum, lyophilized to dryness to give N-(5-(2-(1,4-oxazepan-4-yl)acetamido)-2-methylpyridin-3-yl)-2-(1-methyl-1H-pyrazol-4-yl)pyrazolo[5,1-b]thiazole-7-carboxamide (57.9 mg, 98%) as a white solid. LCMS (ESI): mass for C₂₃H₂₆N₈O₃S: 494.6; m/z found: 495.5[M+H]⁺. ¹H NMR (400 MHz, DMSO-d₆) δ 10.00 (s, 1H), 9.95 (s, 1H), 8.68-8.61 (m, 2H), 8.57 (s, 1H), 8.29-8.21 (m, 2H), 7.94 (s, 1H), 3.94 (s, 3H), 3.78 (t, J=6.0 Hz, 2H), 3.75-3.68 (m, 2H), 3.40 (br s, 2H), 2.91-2.81 (m, 4H), 2.47 (s, 3H), 1.92 (quin, J=5.8 Hz, 2H)

### Example 81: N-(5-(2-(1-oxa-7-azaspiro[3.5]nonan-7-yl)acetamido)-2-methylpyridin-3-yl)-2-(1-methyl-1H-pyrazol-4-yl)pyrazolo[5,1-b]thiazole-7-carboxamide

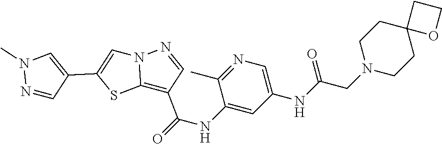 [SMILES: Cc1ncc(NC(=O)CN2CCC3(CCO3)CC2)cc1NC(=O)c1cnn2cc(-c3cnn(C)c3)sc12]

To a solution of N-(5-(2-chloroacetamido)-2-methylpyridin-3-yl)-2-(1-methyl-1H-pyrazol-4-yl)pyrazolo[5,1-b]thiazole-7-carboxamide (90 mg, 0.21 mmol) in DMF (4 mL) was added 1-oxa-7-azaspiro[3.5]nonane (40 mg, 0.31 mmol), potassium carbonate (87 mg, 0.62 mmol) and sodium iodide (19 mg, 0.13 mmol) at room temperature. The resulting mixture was stirred at 50° C. for 2 h before cooling to room temperature. The reaction mixture was filtered and the filtrate was concentrated under vacuum to give crude product, which was purified by preparative high-performance liquid chromatography over column: Phenomenex Gemini-NX 80*40 mm*3 um to give the title compound N-(5-(2-(1-oxa-7-azaspiro[3.5]nonan-7-yl)acetamido)-2-methylpyridin-3-yl)-2-(1-methyl-1H-pyrazol-4-yl)pyrazolo[5,1-b]thiazole-7-carboxamide (44 mg, 40%) as a white solid. LCMS (ESI): mass calcd. for C₂₅H₂₈N₈O₃S 520.6; m/z found, 521.3 [M+H]⁺. ¹H NMR (400 MHz, DMSO-d₆) δ 9.90 (br s, 2H), 8.64-8.53 (m, 2H), 8.49 (s, 1H), 8.17 (br d, J=9.9 Hz, 2H), 7.87 (s, 1H), 4.36 (br t, J=7.6 Hz, 2H), 3.88 (s, 3H), 3.11 (s, 2H), 2.55 (br d, J=5.0 Hz, 2H), 2.40 (s, 3H), 2.38-2.28 (m, 4H), 1.84 (br s, 4H).

### Example 82: N-(5-(2-(2,2-dimethylazetidin-1-yl)acetamido)-2-methylpyridin-3-yl)-2-(1-methyl-1H-pyrazol-4-yl)pyrazolo[5,1-b]thiazole-7-carboxamide

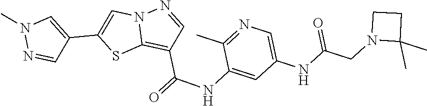

To a solution of N-(5-(2-chloroacetamido)-2-methylpyridin-3-yl)-2-(1-methyl-1H-pyrazol-4-yl)pyrazolo[5,1-b]thiazole-7-carboxamide (100 mg, 0.23 mmol) in DMF (5 mL) was added 2,2-dimethylazetidine (41 mg, 0.34 mmol), potassium carbonate (94 mg, 0.68 mmol) and sodium iodide (20 mg, 0.14 mmol) at room temperature. The resulting mixture was stirred at 50° C. for 2 h before cooling to room temperature. The reaction mixture was filtered and the filtrate was concentrated under vacuum to give crude product, which was purified by preparative high-performance liquid chromatography over column: Phenomenex Gemini-NX C18 75*30 mm*3 um to give the title compound N-(5-(2-(2,2-dimethylazetidin-1-yl)acetamido)-2-methylpyridin-3-yl)-2-(1-methyl-1H-pyrazol-4-yl)pyrazolo[5,1-b]thiazole-7-carboxamide (61.3 mg, 55%) as a white solid. LCMS (ESI): mass calcd. for C₂₃H₂₆N₈O₂S 478.6; m/z found, 479.2 [M+H]⁺. ¹H NMR (400 MHz, DMSO-d₆) δ 9.90 (s, 1H), 9.84 (br s, 1H), 8.60-8.55 (m, 2H), 8.50 (s, 1H), 8.19 (s, 1H), 8.16 (d, J=2.2 Hz, 1H), 7.88 (s, 1H), 3.88 (s, 3H), 3.22 (t, J=7.1 Hz, 2H), 3.15 (s, 2H), 2.39 (s, 3H), 1.88 (t, J=6.9 Hz, 2H), 1.19 (s, 6H).

### Example 83: N-(5-(2-(4-methoxypiperidin-1-yl)acetamido)-2-methylpyridin-3-yl)-2-(1-methyl-1H-pyrazol-4-yl)pyrazolo[5,1-b]thiazole-7-carboxamide

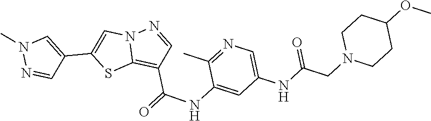

To a solution of N-(5-(2-(4-methoxypiperidin-1-yl)acetamido)-2-methylpyridin-3-yl)-2-(1-methyl-1H-pyrazol-4-yl)pyrazolo[5,1-b]thiazole-7-carboxamide (80 mg, 0.18 mmol), potassium carbonate (73.5 mg, 0.53 mmol), sodium iodide (15.9 mg, 0.11 mmol) in N,N-dimethylformamide (4 mL) was added 4-methoxypiperidine (24.4 mg, 0.21 mmol). The mixture stirred at 50° C. for 2 h and then concentrated under vacuum to give the crude product, which was purified by preparative high-performance liquid chromatography over column: Boston Green ODS 150*30 mm*5 um to give the title compound N-(5-(2-(4-methoxypiperidin-1-yl)acetamido)-2-methylpyridin-3-yl)-2-(1-methyl-1H-pyrazol-4-yl)pyrazolo[5,1-b]thiazole-7-carboxamide (55.2 mg, 54%) as a yellow solid. LCMS (ESI): mass calcd. for C₂₄H₂₈N₈O₃S, 508.596; m/z found, 509.4 [M+H]⁺. ¹H NMR (400 MHz, METHANOL-d₄) δ 8.59 (d, J=2.0 Hz, 1H), 8.36 (br s, 1H), 8.25 (br d, J=2.0 Hz, 1H), 8.22 (s, 1H), 8.02 (s, 1H), 7.81 (s, 1H), 3.94 (s, 3H), 3.57 (s, 2H), 3.45-3.39 (m, 1H), 3.36 (s, 3H), 3.08 (br t, J=7.8 Hz, 2H), 2.78 (br s, 2H), 2.50 (s, 3H), 2.09-1.96 (m, 2H), 1.82 (br s, 2H).

### Example 84: (S)-2-(1-(2-hydroxyethyl)-1H-pyrazol-4-yl)-N-(2-methyl-5-(2-(2-methylpyrrolidin-1-yl)acetamido)pyridin-3-yl)pyrazolo[5,1-b]thiazole-7-carboxamide

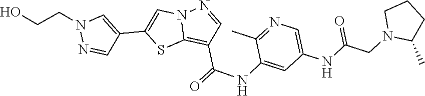 [SMILES: Cc1ncc(NC(=O)CN2CCC[C@@H]2C)cc1NC(=O)c1cnn2cc(-c3cnn(CCO)c3)sc12]

### Step a: (S)-2-bromo-N-(2-methyl-5-(2-(2-methylpyrrolidin-1-yl)acetamido)pyridin-3-yl)pyrazolo[5,1-b]thiazole-7-carboxamide

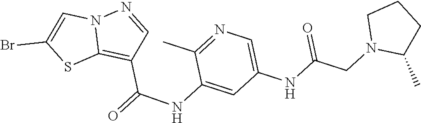 [SMILES: Cc1ncc(NC(=O)CN2CCC[C@@H]2C)cc1NC(=O)c1cnn2cc(Br)sc12]

To a mixture of 2-bromo-N-(5-(2-chloroacetamido)-2-methylpyridin-3-yl)pyrazolo[5,1-b]thiazole-7-carboxamide (400 mg, 0.93 mmol), Cs₂CO₃ (912 mg, 2.8 mmoL) and NaI (84 mg, 0.56 mmol) in DMF (5 mL) was added (S)-2-methylpyrrolidine hydrochloride (136 mg, 1.1 mmol). The reaction mixture was stirred at 50° C. for 2 h before cooling to room temperature. The reaction mixture was filtered and the filtrate was concentrated under vacuum to give crude product, which was purified by column chromatography over silica gel (eluent: petroleum ether:ethyl acetate=1:1) to give the title compound (S)-2-bromo-N-(2-methyl-5-(2-(2-methylpyrrolidin-1-yl)acetamido)pyridin-3-yl)pyrazolo[5,1-b]thiazole-7-carboxamide (200 mg, 40%) as a yellow solid. LCMS (ESI): mass calcd. for C₁₉H₂₁BrN₆O₂S, 477.3; m/z found, 479.1 [M+2]⁺.

### Step b: (S)-2-(1-(2-hydroxyethyl)-1H-pyrazol-4-yl)-N-(2-methyl-5-(2-(2-methylpyrrolidin-1-yl)acetamido)pyridin-3-yl)pyrazolo[5,1-b]thiazole-7-carboxamide

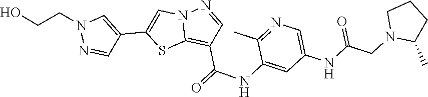 [SMILES: Cc1ncc(NC(=O)CN2CCC[C@@H]2C)cc1NC(=O)c1cnn2cc(-c3cnn(CCO)c3)sc12]

To a solution of (S)-2-bromo-N-(2-methyl-5-(2-(2-methylpyrrolidin-1-yl)acetamido)pyridin-3-yl)pyrazolo[5,1-b]thiazole-7-carboxamide (200 mg, 0.37 mmol), 2-(4-(4,4,5,5-tetramethyl-1,3,2-dioxaborolan-2-yl)-1H-pyrazol-1-yl)ethanol (108 mg, 0.45 mmol) and K₃PO₄ (240 mg, 1.1 mmol) in dioxane/water (5 mL, 4/1) was added PdCl₂(dppf)·CH₂Cl₂ (185 mg, 0.26 mmol) at room temperature under an atmosphere of nitrogen. The reaction vessel was gradually warmed to 100° C. over the course of 10 min, after which time stirring was continued for 12 hours. The reaction mixture was filtered, and the filter cake was rinsed with 5 mL of dichloromethane. The filtrate was collected, dried in vacuum to give the crude product, which was purified by preparative high-performance liquid chromatography over column: Phenomenex Synergi C18 150*30 mm*4 um to give the title compound (S)-2-(1-(2-hydroxyethyl)-1H-pyrazol-4-yl)-N-(2-methyl-5-(2-(2-methylpyrrolidin-1-yl)acetamido)pyridin-3-yl)pyrazolo[5,1-b]thiazole-7-carboxamide (Formic acid salt, 65.8 mg, 33%) as a white solid. LCMS (ESI): mass calcd. for C₂₄H₂₈N₈O₃S, 508.6; m/z found, 509.5. [M+H]⁺. ¹H NMR (400 MHz, METHANOL-d₄) δ 8.62 (d, J=2.27 Hz, 1H), 8.54 (br s, 1H), 8.42 (s, 1H), 8.25 (s, 2H), 8.10 (s, 1H), 7.88 (s, 1H), 4.30 (t, J=5.30 Hz, 2H), 3.94 (t, J=5.25 Hz, 2H), 3.71 (br d, J=16.21 Hz, 1H), 3.37 (br s, 2H), 2.80 (br s, 1H), 2.57 (br d, J=9.18 Hz, 1H), 2.52 (s, 3H), 2.04-2.14 (m, 1H), 1.84-1.98 (m, 2H), 1.54-1.64 (m, 1H), 1.23 (d, J=6.08 Hz, 3H).

### Example 85: (S)-2-(1-(2-hydroxyethyl)-1H-pyrazol-4-yl)-N-(2-methyl-5-(2-(2-methylpiperidin-1-yl)acetamido)pyridin-3-yl)pyrazolo[5,1-b]thiazole-7-carboxamide

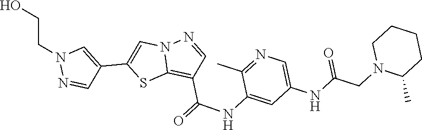 [SMILES: Cc1ncc(NC(=O)CN2CCCC[C@@H]2C)cc1NC(=O)c1cnn2cc(-c3cnn(CCO)c3)sc12]

### Step a: (S)-2-bromo-N-(2-methyl-5-(2-(2-methylpiperidin-1-yl)acetamido)pyridin-3-yl)pyrazolo[5,1-b]thiazole-7-carboxamide

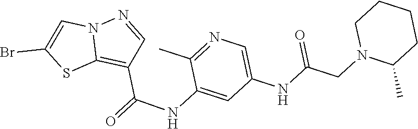 [SMILES: Cc1ncc(NC(=O)CN2CCCC[C@@H]2C)cc1NC(=O)c1cnn2cc(Br)sc12]

To a solution of 2-bromo-N-(5-(2-chloroacetamido)-2-methylpyridin-3-yl)pyrazolo[5,1-b]thiazole-7-carboxamide (120 mg, 0.28 mmol), potassium carbonate (116 mg, 0.84 mmol), sodium iodide (25.2 mg, 0.17 mmol) in N,N-dimethylformamide (3 mL) was added (S)-2-methylpiperidine (33.3 mg, 0.34 mmol). The resulting mixture was stirred at 60° C. for 2 h before cooled to 25° C. The resulting mixture was quenched with cooled H₂O and extracted with ethyl acetate (100 mL*3). The organic extracts were dried over anhydrous Na₂SO₄, and filtered, the filtrate was concentrated under reduced pressure to give crude product, which was purified by column chromatography over silica gel (eluent: petroleum ether/ethyl acetate=0:1) to give the title compound (S)-2-bromo-N-(2-methyl-5-(2-(2-methylpiperidin-1-yl)acetamido)pyridin-3-yl)pyrazolo[5,1-b]thiazole-7-carboxamide (100 mg, 73%) as a white solid.

### Step b: (S)-2-(1-(2-hydroxyethyl)-1H-pyrazol-4-yl)-N-(2-methyl-5-(2-(2-methylpiperidin-1-yl)acetamido)pyridin-3-yl)pyrazolo[5,1-b]thiazole-7-carboxamide

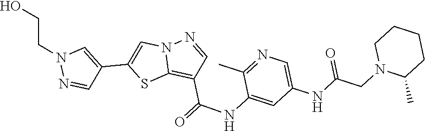 [SMILES: Cc1ncc(NC(=O)CN2CCCC[C@@H]2C)cc1NC(=O)c1cnn2cc(-c3cnn(CCO)c3)sc12]

To a solution of (S)-2-bromo-N-(2-methyl-5-(2-(2-methylpiperidin-1-yl)acetamido)pyridin-3-yl)pyrazolo[5,1-b]thiazole-7-carboxamide (100 mg, 0.20 mmol) and 2-(4-(4,4,5,5-tetramethyl-1,3,2-dioxaborolan-2-yl)-1H-pyrazol-1-yl)ethanol (55.6 mg, 0.23 mmol) in 1,4-dioxane (4 mL) and H₂O (1 mL) was added [1,1-Bis(diphenylphosphino)ferrocene]palladium(II) chloride dichloromethane complex (31.8 mg, 0.04 mmol) and K₃PO₄ (124 mg, 0.58 mmol) under N₂. The resulting mixture was stirred at 90° C. for 3 h before cooled to 25° C. The reaction mixture was diluted with ethyl acetate (10 mL) and filtered over celite. The filtrate was concentrated to the crude product, which was treated with dichloromethane (10 mL) and water (10 mL). The organic phase was concentrated under reduced pressure to give the crude product, which was purified by preparative high-performance liquid chromatography over column: Boston Green ODS 150*30 mm*5 um to give the title compound (S)-2-(1-(2-hydroxyethyl)-1H-pyrazol-4-yl)-N-(2-methyl-5-(2-(2-methylpiperidin-1-yl)acetamido)pyridin-3-yl)pyrazolo[5,1-b]thiazole-7-carboxamide (31.2 mg, 30%) as a yellow solid. LCMS (ESI): mass calcd. for C₂₅H₃₀N₈O₃S, 522.623; m/z found, 523.3 [M+H]⁺. ¹H NMR (400 MHz, METHANOL-d₄) δ 8.58 (d, J=2.2 Hz, 1H), 8.38 (s, 1H), 8.24-8.21 (m, 2H), 8.07 (s, 1H), 7.84 (s, 1H), 4.26 (t, J=5.2 Hz, 2H), 3.90 (t, J=5.3 Hz, 2H), 3.51 (br d, J=15.2 Hz, 1H), 3.16 (br d, J=16.1 Hz, 1H), 2.95 (br s, 1H), 2.48 (s, 5H), 1.75-1.62 (m, 4H), 1.46-1.33 (m, 2H), 1.12 (d, J=6.2 Hz, 3H).

### Example 86: N-(5-(2-(4-azaspiro[2.5]octan-4-yl)acetamido)-2-methylpyridin-3-yl)-2-(1-(2-hydroxyethyl)-1H-pyrazol-4-yl)pyrazolo[5,1-b]thiazole-7-carboxamide

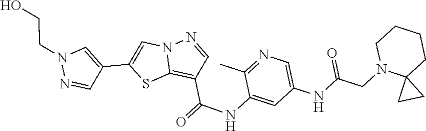 [SMILES: Cc1ncc(NC(=O)CN2CCCCC23CC3)cc1NC(=O)c1cnn2cc(-c3cnn(CCO)c3)sc12]

### Step a: N-(5-(2-(4-azaspiro[2.5]octan-4-yl)acetamido)-2-methylpyridin-3-yl)-2-bromopyrazolo[5,1-b]thiazole-7-carboxamide

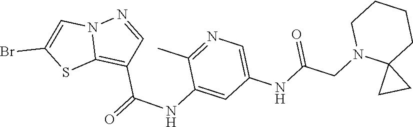 [SMILES: Cc1ncc(NC(=O)CN2CCCCC23CC3)cc1NC(=O)c1cnn2cc(Br)sc12]

To a solution of 2-bromo-N-(5-(2-chloroacetamido)-2-methylpyridin-3-yl)pyrazolo[5,1-b]thiazole-7-carboxamide (250 mg, 0.56 mmol), cesium carbonate (545 mg, 1.67 mmol), sodium iodide (50.1 mg, 0.33 mmol) in N,N-dimethylformamide (3 mL) was added 4-azaspiro[2.5]octane hydrochloride (123 mg, 0.67 mmol). The resulting mixture was stirred at 60° C. for 2 h before cooled to 25° C. The resulting mixture was quenched with cooled H₂O and extracted with ethyl acetate (10 mL*3). The organic extracts were dried over anhydrous Na₂SO₄, and filtered, the filtrate was concentrated under reduced pressure to give crude product, which was purified by column chromatography over silica gel (eluent: petroleum ether/ethyl acetate=0:1) to give the title compound N-(5-(2-(4-azaspiro[2.5]octan-4-yl)acetamido)-2-methylpyridin-3-yl)-2-bromopyrazolo[5,1-b]thiazole-7-carboxamide (120 mg, 43%) as a white solid.

### Step b: N-(5-(2-(4-azaspiro[2.5]octan-4-yl)acetamido)-2-methylpyridin-3-yl)-2-(1-(2-hydroxyethyl)-1H-pyrazol-4-yl)pyrazolo[5,1-b]thiazole-7-carboxamide

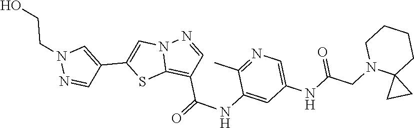 [SMILES: Cc1ncc(NC(=O)CN2CCCCC23CC3)cc1NC(=O)c1cnn2cc(-c3cnn(CCO)c3)sc12]

To a solution of N-(5-(2-(4-azaspiro[2.5]octan-4-yl)acetamido)-2-methylpyridin-3-yl)-2-bromopyrazolo[5,1-b]thiazole-7-carboxamide (120 mg, 0.24 mmol) and 2-(4-(4,4,5,5-tetramethyl-1,3,2-dioxaborolan-2-yl)-1H-pyrazol-1-yl)ethanol (68.1 mg, 0.29 mmol) in 1,4-dioxane (4 mL) and H₂O (1 mL) was added [1,1-Bis(diphenylphosphino)ferrocene]palladium(II) chloride dichloromethane complex (38.9 mg, 0.05 mmol) and K₃PO₄ (152 mg, 0.72 mmol) under N₂. The resulting mixture was stirred at 90° C. for 3 h before cooled to 25° C. The reaction mixture was diluted with ethyl acetate (10 mL) and filtered over celite. The filtrate was concentrated to the crude product, which was treated with dichloromethane (10 mL) and water (10 mL). The organic phase was concentrated under reduced pressure to give the crude product, which was purified by preparative high-performance liquid chromatography over column: Boston Green ODS 150*30 mm*5 um to give the title compound N-(5-(2-(4-azaspiro[2.5]octan-4-yl)acetamido)-2-methylpyridin-3-yl)-2-(1-(2-hydroxyethyl)-1H-pyrazol-4-yl)pyrazolo[5,1-b]thiazole-7-carboxamide (31.2 mg, 30%) as a yellow solid. LCMS (ESI): mass calcd. for C₂₆H₃₀N₈O₃S, 534.633; m/z found, 535.4 [M+H]⁺. ¹H NMR (400 MHz, METHANOL-d₄) δ 8.59 (d, J=2.2 Hz, 1H), 8.39 (s, 1H), 8.24 (s, 1H), 8.22 (d, J=2.2 Hz, 1H), 8.09 (s, 1H), 7.87 (s, 1H), 4.28 (t, J=5.2 Hz, 2H), 3.92 (t, J=5.2 Hz, 2H), 3.57 (s, 2H), 2.97 (br t, J=5.4 Hz, 2H), 2.50 (s, 3H), 1.80-1.71 (m, 2H), 1.59 (br s, 2H), 1.52-1.27 (m, 2H), 0.82-0.76 (m, 2H), 0.45 (s, 2H).

### Example 87: N-(5-(2-(4-azaspiro[2.4]heptan-4-yl)acetamido)-2-methylpyridin-3-yl)-2-(1-(2-hydroxyethyl)-1H-pyrazol-4-yl)pyrazolo[5,1-b]thiazole-7-carboxamide

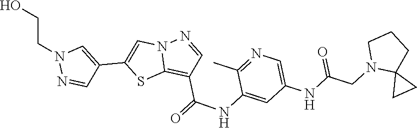 [SMILES: Cc1ncc(NC(=O)CN2CCCC23CC3)cc1NC(=O)c1cnn2cc(-c3cnn(CCO)c3)sc12]

### Step a: tert-butyl (5-(2-(1-(2-hydroxyethyl)-1H-pyrazol-4-yl)pyrazolo[5,1-b]thiazole-7-carboxamido)-6-methylpyridin-3-yl)carbamate

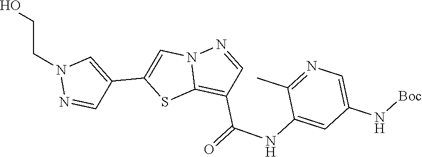 [SMILES: Cc1ncc(NC(=O)OC(C)(C)C)cc1NC(=O)c1cnn2cc(-c3cnn(CCO)c3)sc12]

To a solution of tert-butyl (5-(2-bromopyrazolo[5,1-b]thiazole-7-carboxamido)-6-methylpyridin-3-yl)carbamate (1.5 g, 2.8 mmol) and 2-(4-(4,4,5,5-tetramethyl-1,3,2-dioxaborolan-2-yl)-1H-pyrazol-1-yl)ethanol (0.81 g, 3.3 mmol) in 1,4-dioxane (16 mL) and H₂O (4 mL) was added [1,1-Bis(diphenylphosphino)ferrocene]palladium(II) chloride dichloromethane complex (460 mg, 0.56 mmol) and potassium phosphate (1.8 g, 8.4 mmol) under N₂. The resulting mixture was stirred at 90° C. for 3 h before cooled to 25° C. The resulting mixture was quenched with cooled H₂O and extracted with ethyl acetate (100 mL*3). The organic extracts were dried over anhydrous Na₂SO₄, and filtered, the filtrate was concentrated under reduced pressure to give crude product, which was purified by column chromatography over silica gel (eluent: petroleum ether/ethyl acetate=0:1) to give the title compound tert-butyl (5-(2-(1-(2-hydroxyethyl)-1H-pyrazol-4-yl)pyrazolo[5,1-b]thiazole-7-carboxamido)-6-methylpyridin-3-yl)carbamate (2.4 g, 47%) as a yellow solid. LCMS (ESI): mass calcd. for C₂₂H₂₅N₇O₄S, 483.543; m/z found, 484.2 [M+H]⁺.

### Step b: N-(5-amino-2-methylpyridin-3-yl)-2-(1-(2-hydroxyethyl)-1H-pyrazol-4-yl)pyrazolo[5,1-b]thiazole-7-carboxamide

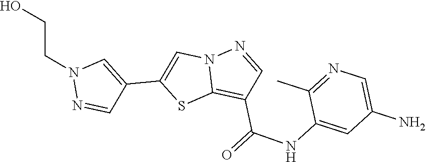 [SMILES: Cc1ncc(N)cc1NC(=O)c1cnn2cc(-c3cnn(CCO)c3)sc12]

To a solution of tert-butyl (5-(2-(1-(2-hydroxyethyl)-1H-pyrazol-4-yl)pyrazolo[5,1-b]thiazole-7-carboxamido)-6-methylpyridin-3-yl)carbamate (400 mg, 0.83 mmol) in DCM (1 mL) was added HCl/dioxane (1 mL, 4M) at 0° C. The resultant mixture was stirred at 20° C. for 1 hour. LCMS showed the reaction was completed. Then the reaction mixture was concentrated under reduced pressure to afford the crude product N-(5-amino-2-methylpyridin-3-yl)-2-(1-methyl-1H-pyrazol-4-yl)pyrazolo[5,1-b]thiazole-7-carboxamide (195 mg, 50%) as a yellow solid. LCMS (ESI): mass calcd. for C₁₇H₁₇N₇O₂S, 383.428; m/z found, 384.1 [M+H]⁺.

### Step c: N-(5-(2-chloroacetamido)-2-methylpyridin-3-yl)-2-(1-(2-hydroxyethyl)-1H-pyrazol-4-yl)pyrazolo[5,1-b]thiazole-7-carboxamide

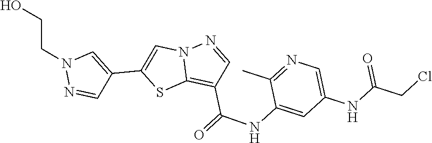 [SMILES: Cc1ncc(NC(=O)CCl)cc1NC(=O)c1cnn2cc(-c3cnn(CCO)c3)sc12]

To a solution of N-(5-amino-2-methylpyridin-3-yl)-2-(1-(2-hydroxyethyl)-1H-pyrazol-4-yl)pyrazolo[5,1-b]thiazole-7-carboxamide (195 mg, 0.43 mmol), sodium hydrogencarbonate (108 mg, 1.3 mmol) in N,N-dimethylformamide (3 mL) was added 2-chloroacetyl chloride (57.9 mg, 0.51 mmol). The mixture stirred at 25° C. for 2 h and then concentrated under vacuum to give the crude product. Then H₂O (10 mL) was added. The mixture was stirred at room temperature for 0.5 h and filtered. The filter cake was washed with H₂O (10 mL). The filter cake was dried in vacuo to give the title compound N-(5-(2-chloroacetamido)-2-methylpyridin-3-yl)-2-(1-(2-hydroxyethyl)-1H-pyrazol-4-yl)pyrazolo[5,1-b]thiazole-7-carboxamide (75 mg, 38%) as a yellow solid. LCMS (ESI): mass calcd. for C₁₉H₁₈ClN₇O₃S, 459.909; m/z found, 460.1 [M+H]⁺.

### Step d: N-(5-(2-(4-azaspiro[2.4]heptan-4-yl)acetamido)-2-methylpyridin-3-yl)-2-(1-(2-hydroxyethyl)-1H-pyrazol-4-yl)pyrazolo[5,1-b]thiazole-7-carboxamide

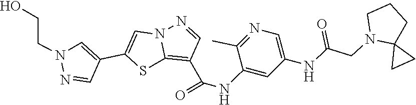 [SMILES: Cc1ncc(NC(=O)CN2CCCC23CC3)cc1NC(=O)c1cnn2cc(-c3cnn(CCO)c3)sc12]

To a solution of N-(5-(2-chloroacetamido)-2-methylpyridin-3-yl)-2-(1-(2-hydroxyethyl)-1H-pyrazol-4-yl)pyrazolo[5,1-b]thiazole-7-carboxamide (60 mg, 0.13 mmol), potassium carbonate (54 mg, 0.39 mmol), sodium iodide (11 mg, 0.08 mmol) in N,N-dimethylformamide (5 mL) was added 4-azaspiro[2.4]heptane hydrochloride (21 mg, 0.16 mmol). The mixture stirred at 60° C. for 1 h and then concentrated under vacuum to give the crude product, which was purified by preparative high-performance liquid chromatography over column: Boston Green ODS 150*30 mm*5 um to give the title compound N-(5-(2-(4-azaspiro[2.4]heptan-4-yl)acetamido)-2-methylpyridin-3-yl)-2-(1-(2-hydroxyethyl)-1H-pyrazol-4-yl)pyrazolo[5,1-b]thiazole-7-carboxamide (47.1 mg, 67%) as a yellow solid. LCMS (ESI): mass calcd. for C₂₅H₂₈N₈O₃S, 520.607; m/z found, 521.3 [M+H]⁺. ¹H NMR (400 MHz, METHANOL-d₄) δ 8.59 (d, J=2.2 Hz, 1H), 8.39 (s, 1H), 8.25-8.21 (m, 2H), 8.08 (s, 1H), 7.86 (s, 1H), 4.28 (t, J=5.3 Hz, 2H), 3.92 (t, J=5.3 Hz, 2H), 3.26 (s, 2H), 3.04 (t, J=7.1 Hz, 2H), 2.50 (s, 3H), 2.05-1.97 (m, 2H), 1.91-1.85 (m, 2H), 0.89-0.84 (m, 2H), 0.57-0.52 (m, 2H).

### Example 88: N-(5-(3-(2,2-dimethylpyrrolidin-1-yl)propanamido)-2-methylpyridin-3-yl)-2-(1-(2-hydroxyethyl)-1H-pyrazol-4-yl)pyrazolo[5,1-b]thiazole-7-carboxamide

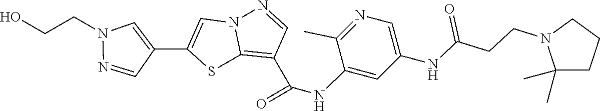 [SMILES: Cc1ncc(NC(=O)CCN2CCCC2(C)C)cc1NC(=O)c1cnn2cc(-c3cnn(CCO)c3)sc12]

To a solution of 2-bromo-N-(5-(3-(2,2-dimethylpyrrolidin-1-yl)propanamido)-2-methylpyridin-3-yl) pyrazolo[5,1-b]thiazole-7-carboxamide (200 mg, 0.40 mmol), 2-(4-(4,4,5,5-tetramethyl-1,3,2-dioxaborolan-2-yl)-1H-pyrazol-1-yl)ethanol (120 mg, 0.50 mmol) and Cs₂CO₃ (400 mg, 1.2 mmol) in 1,4-dioxane (10 mL) was added Pd(dppf)Cl₂·CH₂Cl₂ (60 mg, 0.08 mmol) under N₂, the yellow mixture stirred at 100° C. for 2 hours. The mixture was cooled to 25° C., and then diluted with ethyl acetate (30 mL), filtered and the filtrate was concentrated to remove solvent to give a residue. The crude product was purified by preparative high-performance liquid chromatography over column: Welch Xtimate C18 150*30 mm*5 um to give the title compound: N-(5-(3-(2,2-dimethylpyrrolidin-1-yl)propanamido)-2-methylpyridin-3-yl)-2-(1-(2-hydroxyethyl)-1H-pyrazol-4-yl)pyrazolo[5,1-b]thiazole-7-carboxamide (35 mg, 16%) as a white solid. LCMS (ESI): mass calcd. for C₂₆H₃₂N₈O₃S, 536.6; m/z found, 537.2 [M+H]⁺. ¹H NMR (400 MHz, DMSO-d₆) δ ppm 10.33 (s, 1H), 9.87 (s, 1H), 8.59 (s, 1H), 8.43-8.54 (m, 2H), 8.21 (s, 1H), 8.13 (d, J=2.01 Hz, 1H), 7.91 (s, 1H), 4.96 (t, J=4.89 Hz, 1H), 4.18 (t, J=5.52 Hz, 2H), 3.72-3.82 (m, 2H), 2.62-2.77 (m, 4H), 2.44 (br d, J=6.78 Hz, 2H), 2.38-2.42 (m, 3H), 1.62-1.75 (m, 2H), 1.49-1.60 (m, 2H), 0.95 (s, 6H).

### Example 89: N-(5-(3-(3,3-dimethylpyrrolidin-1-yl)propanamido)-2-methylpyridin-3-yl)-2-(1-(2-methoxyethyl)-1H-pyrazol-4-yl)pyrazolo[5,1-b]thiazole-7-carboxamide

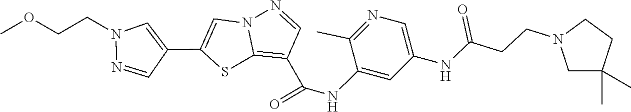

To the mixture of N-(5-(3-chloropropanamido)-2-methylpyridin-3-yl)-2-(1-(2-methoxyethyl)-1H-pyrazol-4-yl)pyrazolo[5,1-b]thiazole-7-carboxamide (90 mg, 0.17 mmol) in MeCN (5 mL) was added TEA (0.2 mL, 1.4 mmol) and 3,3-dimethylpyrrolidine hydrochloride (92 mg, 0.68 mmol). The resulting mixture was stirred at 70° C. for 16 h and then the reaction mixture was filtered and the filtrate was purified by preparative high-performance liquid chromatography over column: Welch Xtimate C18 150*30 mm*5 um to give the title compound: N-(5-(3-(3,3-dimethylpyrrolidin-1-yl)propanamido)-2-methylpyridin-3-yl)-2-(1-(2-methoxyethyl)-1H-pyrazol-4-yl)pyrazolo[5,1-b]thiazole-7-carboxamide (39 mg, 41%) as a white solid. LCMS (ESI): mass calcd. for C₂₇H₃₄N₈O₃S, 550.7; m/z found, 551.3 [M+H]⁺. ¹H NMR (400 MHz, METHANOL-d₄) δ 8.56 (d, J=2.38 Hz, 1H), 8.41 (s, 1H), 8.26 (s, 1H), 8.24 (d, J=2.38 Hz, 1H), 8.09 (s, 1H), 7.87 (s, 1H), 4.37 (t, J=5.19 Hz, 2H), 3.79 (t, J=5.13 Hz, 2H), 3.36 (s, 3H), 2.84-2.93 (m, 2H), 2.77 (br d, J=5.96 Hz, 2H), 2.60-2.66 (m, 2H), 2.51 (s, 3H), 2.49 (br s, 2H), 1.68 (br t, J=6.91 Hz, 2H), 1.14 (s, 6H).

### Example 90: N-(5-(3-(6-azaspiro[3.4]octan-6-yl)propanamido)-2-methylpyridin-3-yl)-2-(1-(2-methoxyethyl)-1H-pyrazol-4-yl)pyrazolo[5,1-b]thiazole-7-carboxamide

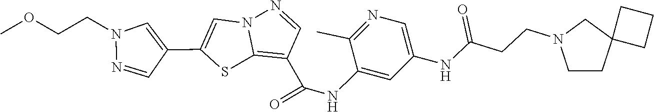 [SMILES: COCCn1cc(-c2cn3ncc(C(=O)Nc4cc(NC(=O)CCN5CCC6(CCC6)C5)cnc4C)c3s2)cn1]

To the mixture of N-(5-(3-chloropropanamido)-2-methylpyridin-3-yl)-2-(1-(2-methoxyethyl)-1H-pyrazol-4-yl)pyrazolo[5,1-b]thiazole-7-carboxamide (90 mg, 0.17 mmol) in MeCN (4 mL) was added TEA (185 μL, 1.33 mmol) and 6-azaspiro[3.4]octane (80 mg, 0.72 mmol). The resulting mixture was stirred at 70° C. for 16 h and then the reaction mixture was filtered and the filtrate was purified by preparative high-performance liquid chromatography over column: Welch Xtimate C18 150*30 mm*5 um to give the title compound: N-(5-(3-(6-azaspiro[3.4]octan-6-yl)propanamido)-2-methylpyridin-3-yl)-2-(1-(2-methoxyethyl)-1H-pyrazol-4-yl)pyrazolo[5,1-b]thiazole-7-carboxamide (37 mg, 40%) as a yellow solid. LCMS (ESI): mass calcd. for C₂₈H₃₄N₈O₃S, 562.7; m/z found, 563.2 [M+H]⁺. ¹H NMR (400 MHz, METHANOL-d₄) δ 8.55 (t, J=2.68 Hz, 1H), 8.41 (s, 1H), 8.26 (d, J=3.46 Hz, 1H), 8.23 (d, J=2.26 Hz, 1H), 8.09 (d, J=1.43 Hz, 1H), 7.87 (d, J=1.31 Hz, 1H), 4.37 (t, J=5.13 Hz, 2H), 3.79 (t, J=5.13 Hz, 2H), 3.36 (s, 3H), 2.81-2.93 (m, 2H), 2.72-2.78 (m, 2H), 2.68 (br t, J=7.03 Hz, 2H), 2.58-2.66 (m, 2H), 2.51 (s, 3H), 2.08 (br t, J=8.76 Hz, 2H), 1.95-2.04 (m, 4H), 1.81-1.94 (m, 2H).

### Example 91: N-(5-(3-(5-azaspiro[2.4]heptan-5-yl)propanamido)-2-methylpyridin-3-yl)-2-(1-(2-methoxyethyl)-1H-pyrazol-4-yl)pyrazolo[5,1-b]thiazole-7-carboxamide

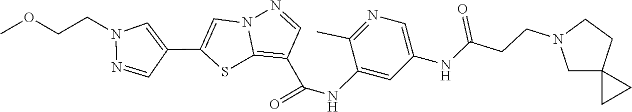 [SMILES: COCCn1cc(-c2cn3ncc(C(=O)Nc4cc(NC(=O)CCN5CCC6(CC6)C5)cnc4C)c3s2)cn1]

To a solution of N-(5-(3-chloropropanamido)-2-methylpyridin-3-yl)-2-(1-(2-methoxyethyl)-1H-pyrazol-4-yl)pyrazolo[5,1-b]thiazole-7-carboxamide (90 mg, 0.17 mmol) in MeCN (4.5 mL) was added 5-azaspiro[2.4]heptane hydrochloride (99 mg, 0.74 mmol) and TEA (0.2 mL, 1.4 mmol) at room temperature. The resulting mixture was stirred at 70° C. for 16 h before cooling to room temperature. The reaction mixture was filtered and the filtrate was concentrated under vacuum to give crude product, which was purified by preparative high-performance liquid chromatography over column: Phenomenex C18 80*40 mm*3 um to give the title compound N-(5-(3-(5-azaspiro[2.4]heptan-5-yl)propanamido)-2-methylpyridin-3-yl)-2-(1-(2-methoxyethyl)-1H-pyrazol-4-yl)pyrazolo[5,1-b]thiazole-7-carboxamide (55 mg, 57%) as a white solid. LCMS (ESI): mass calcd. for C₂₇H₃₂N₈O₃S 548.7; m/z found, 549.3 [M+H]⁺. ¹H NMR (400 MHz, METHANOL-d₄) δ 8.57 (d, J=2.26 Hz, 1H), 8.41 (s, 1H), 8.26 (s, 1H), 8.24 (d, J=2.51 Hz, 1H), 8.09 (s, 1H), 7.87 (s, 1H), 4.37 (t, J=5.14 Hz, 2H), 3.79 (t, J=5.14 Hz, 2H), 3.36 (s, 3H), 2.93 (dt, J=18.01, 7.18 Hz, 4H), 2.63-2.72 (m, 4H), 2.51 (s, 3H), 1.90 (t, J=7.03 Hz, 2H), 0.55-0.70 (m, 4H).

### Example 92: N-(5-(3-((1S,4R)-2-azabicyclo[2.2.1]heptan-2-yl)propanamido)-2-methylpyridin-3-yl)-2-(1-(2-methoxyethyl)-1H-pyrazol-4-yl)pyrazolo[5,1-b]thiazole-7-carboxamide

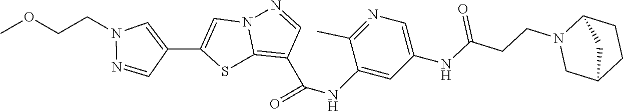 [SMILES: COCCn1cc(-c2cn3ncc(C(=O)Nc4cc(NC(=O)CCN5C[C@@H]6CC[C@H]5C6)cnc4C)c3s2)cn1]

To a solution of N-(5-(3-chloropropanamido)-2-methylpyridin-3-yl)-2-(1-(2-methoxyethyl)-1H-pyrazol-4-yl)pyrazolo[5,1-b]thiazole-7-carboxamide (90 mg, 0.17 mmol) in MeCN (4.5 mL) was added (1S,4R)-2-azabicyclo[2.2.1]heptane hydrochloride (99 mg, 0.74 mmol) and TEA (0.2 mL, 1.4 mmol) at room temperature. The resulting mixture was stirred at 70° C. for 16 h before cooling to room temperature. The reaction mixture was filtered and the filtrate was concentrated under vacuum to give crude product, which was purified by preparative high-performance liquid chromatography over column: Phenomenex C18 150*40 mm*5 um and by Supercritical Fluid Chromatography over column: DAICEL CHIRALCEL OD-H (250 mm*30 mm, 5 um) to give the title compound N-(5-(3-((1S,4R)-2-azabicyclo[2.2.1]heptan-2-yl)propanamido)-2-methylpyridin-3-yl)-2-(1-(2-methoxyethyl)-1H-pyrazol-4-yl)pyrazolo[5,1-b]thiazole-7-carboxamide (28 mg, 31%) as a white solid. LCMS (ESI): mass calcd. for C₂₇H₃₂N₈O₃S 548.7; m/z found, 549.3 [M+H]⁺. ¹H NMR (400 MHz, METHANOL-d₄) δ 8.57 (d, J=2.01 Hz, 1H), 8.41 (s, 1H), 8.27 (s, 1H), 8.24 (d, J=1.76 Hz, 1H), 8.09 (s, 1H), 7.87 (s, 1H), 4.37 (t, J=5.02 Hz, 2H), 3.79 (t, J=5.14 Hz, 2H), 3.50 (br s, 1H), 3.36 (s, 3H), 3.05-3.16 (m, 1H), 2.95 (br s, 2H), 2.65 (br s, 2H), 2.52 (s, 3H), 2.49 (br s, 1H), 1.84 (br d, J=12.30 Hz, 2H), 1.57-1.73 (m, 2H), 1.36-1.52 (m, 2H), 1.31 (s, 1H).

### Example 93: N-(5-(3-(3,3-dimethylazetidin-1-yl)propanamido)-2-methylpyridin-3-yl)-2-(1-(2-methoxyethyl)-1H-pyrazol-4-yl)pyrazolo[5,1-b]thiazole-7-carboxamide

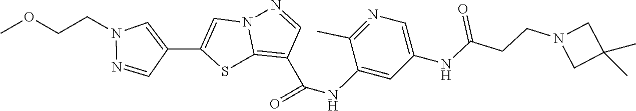

A mixture of N-(5-(3-chloropropanamido)-2-methylpyridin-3-yl)-2-(1-(2-methoxyethyl)-1H-pyrazol-4-yl)pyrazolo[5,1-b]thiazole-7-carboxamide (90 mg, 0.17 mmol) acid in MeCN (5 mL) was added triethylamine (0.21 mL, 1.5 mmol), then added 3,3-dimethylazetidine hydrochloride (90 mg, 0.74 mmol). The mixture stirred at 70° C. for 16 hours and then concentrated under vacuum to give the crude product, which was purified by preparative high-performance liquid chromatography over column: Phenomenex C18 80*40 mm*3 um to give the title compound N-(5-(3-(3,3-dimethylazetidin-1-yl)propanamido)-2-methylpyridin-3-yl)-2-(1-(2-methoxyethyl)-1H-pyrazol-4-yl)pyrazolo[5,1-b]thiazole-7-carboxamide (64 mg, 67%) as a white solid. LCMS (ESI): mass calcd. for C₂₆H₃₂N₈O₃S, 536.6; m/z found, 537.3 [M+H]⁺. ¹HNMR (400 MHz, METHANOL-d₄) δ 8.57 (s, 1H), 8.41 (s, 1H), 8.24 (dd, J=2.7, 9.7 Hz, 2H), 8.08 (s, 1H), 7.87 (s, 1H), 4.37 (t, J=5.1 Hz, 2H), 3.79 (t, J=5.1 Hz, 2H), 3.36 (s, 3H), 3.14 (s, 4H), 2.90 (t, J=7.2 Hz, 2H), 2.53-2.46 (m, 5H), 1.26 (s, 6H).

### Example 94: N-(5-(3-((1R,4S)-2-azabicyclo[2.2.1]heptan-2-yl)propanamido)-2-methylpyridin-3-yl)-2-(1-(2-methoxyethyl)-1H-pyrazol-4-yl)pyrazolo[5,1-b]thiazole-7-carboxamide

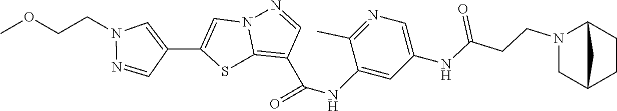 [SMILES: COCCn1cc(-c2cn3ncc(C(=O)Nc4cc(NC(=O)CCN5C[C@H]6CC[C@@H]5C6)cnc4C)c3s2)cn1]

To a solution of N-(5-(3-chloropropanamido)-2-methylpyridin-3-yl)-2-(1-(2-methoxyethyl)-1H-pyrazol-4-yl)pyrazolo[5,1-b]thiazole-7-carboxamide (100 mg, 0.19 mmol) in MeCN (3 mL) was added (1R,4S)-2-azabicyclo[2.2.1]heptane hydrochloride (120 mg, 0.90 mmol), triethylamine (0.23 mL, 1.7 mmol). The resulting mixture was stirred at 70° C. for 16 h. The resulting mixture was purified by preparative high-performance liquid chromatography over column: Phenomenex Gemini-NX 75*30 mm*3 um to give the crude product and the crude product was purified by Supercritical Fluid Chromatography over column: DAICEL CHIRALPAK AS 250 mm*30 mm, 5 um to give title compound N-(5-(3-((1R,4S)-2-azabicyclo[2.2.1]heptan-2-yl)propanamido)-2-methylpyridin-3-yl)-2-(1-(2-methoxyethyl)-1H-pyrazol-4-yl)pyrazolo[5,1-b]thiazole-7-carboxamide (34.2 mg, 32%) as a white solid. LCMS (ESI): mass calcd. for C₂₇H₃₂N₈O₃S, 548.7; m/z found, 549.3 [M+H]⁺. ¹H NMR (400 MHz, CHLOROFORM-d) δ 11.78 (br s, 1H), 8.48 (d, J=2.03 Hz, 1H), 8.30 (d, J=2.03 Hz, 1H), 7.99 (s, 1H), 7.74 (s, 1H), 7.65 (s, 1H), 7.64 (s, 1H), 7.41 (s, 1H), 4.27 (t, J=5.01 Hz, 2H), 3.71 (t, J=5.07 Hz, 2H), 3.30 (s, 3H), 2.80-2.89 (m, 1H), 2.71-2.80 (m, 2H), 2.47 (s, 3H), 2.41 (br d, J=3.34 Hz, 2H), 2.34-2.40 (m, 2H), 1.72 (br s, 1H), 1.46-1.54 (m, 2H), 1.32-1.41 (m, 2H), 1.16-1.32 (m, 2H).

### Example 95: N-(5-(3-(1-azaspiro[3.3]heptan-1-yl)propanamido)-2-methylpyridin-3-yl)-2-(1-(2-methoxyethyl)-1H-pyrazol-4-yl)pyrazolo[5,1-b]thiazole-7-carboxamide

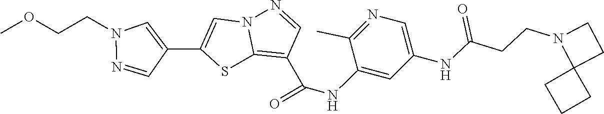 [SMILES: COCCn1cc(-c2cn3ncc(C(=O)Nc4cc(NC(=O)CCN5CCC56CCC6)cnc4C)c3s2)cn1]

To a mixture of N-(5-(3-chloropropanamido)-2-methylpyridin-3-yl)-2-(1-(2-methoxyethyl)-1H-pyrazol-4-yl)pyrazolo[5,1-b]thiazole-7-carboxamide (100 mg, 0.188 mmol) in MeCN (5 mL) was added aspiro[3.3]heptane oxalate (150 mg, 0.801 mmol), TEA (210 μL, 1.527 mmol). The reaction mixture was stirred at 70° C. for 1.5 hours. The reaction mixture was concentrated under vacuum to give crude product, which was purified by preparative high-performance liquid chromatography over column: Phenomenex C18 80*40 mm*3 um and SFC over column: DAICEL CHIRALCEL OJ-H (250 mm*30 mm, 5 um) to give the final compound N-(5-(3-(1-azaspiro[3.3]heptan-1-yl)propanamido)-2-methylpyridin-3-yl)-2-(1-(2-methoxyethyl)-1H-pyrazol-4-yl)pyrazolo[5,1-b]thiazole-7-carboxamide (30.6 mg, 97%) as a white solid. LCMS (ESI): mass for C₂₇H₃₂N₈O₃S: 548.6; m/z found: 549.3 [M+H]⁺. ¹H NMR (400 MHz, CD₃OD) δ 8.63-8.49 (m, 1H), 8.44-8.36 (m, 1H), 8.30-8.18 (m, 2H), 8.15-8.00 (m, 1H), 7.90-7.77 (m, 1H), 4.46-4.25 (m, 2H), 3.86-3.72 (m, 2H), 3.35 (s, 3H), 3.22-3.11 (m, 2H), 2.90-2.78 (m, 2H), 2.55-2.45 (m, 5H), 2.40-2.28 (m, 2H), 2.27-2.19 (m, 2H), 2.04-1.91 (m, 2H), 1.74-1.61 (m, 2H).

### Example 96: N-(5-(3-(3,3-dimethylazetidin-1-yl)propanamido)-2-methylpyridin-3-yl)-2-(1-methyl-1H-pyrazol-4-yl)pyrazolo[5,1-b]thiazole-7-carboxamide

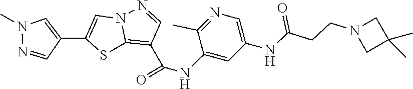

To a solution of N-(5-(3-chloropropanamido)-2-methylpyridin-3-yl)-2-(1-methyl-1H-pyrazol-4-yl)pyrazolo[5,1-b]thiazole-7-carboxamide (50 mg, 0.13 mmol), 3,3-dimethylazetidine (34.1 mg, 0.19 mmol) in Dimethyl sulfoxide (4 mL) was added 2,3,4,6,7,8,9,10-octahydropyrimido[1,2-a]azepine (73.6 mg, 0.19 mmol). The mixture stirred at 60° C. for 1 h and then concentrated under vacuum to give the crude product, which was purified by preparative high-performance liquid chromatography over column: Welch Xtimate C18 150*30 mm*5 um to give the title compound N-(5-(3-(3,3-dimethylazetidin-1-yl)propanamido)-2-methylpyridin-3-yl)-2-(1-methyl-1H-pyrazol-4-yl)pyrazolo[5,1-b]thiazole-7-carboxamide (30.4 mg, 44%) as a white solid. LCMS (ESI): mass calcd. for C₂₄H₂₈N₈O₂S, 492.597; m/z found, 493.3 [M+H]⁺. ¹H NMR (400 MHz, METHANOL-d₄) δ 8.53 (s, 1H), 8.38 (s, 1H), 8.20 (s, 2H), 8.01 (s, 1H), 7.81 (s, 1H), 3.94 (s, 3H), 3.07 (s, 4H), 2.83 (br t, J=7.3 Hz, 2H), 2.52-2.43 (m, 5H), 1.23 (s, 6H).

### Example 97: N-(5-(3-(6-azaspiro[2.5]octan-6-yl)propanamido)-2-methylpyridin-3-yl)-2-(1-methyl-1H-pyrazol-4-yl)pyrazolo[5,1-b]thiazole-7-carboxamide

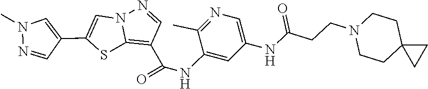 [SMILES: Cc1ncc(NC(=O)CCN2CCC3(CC2)CC3)cc1NC(=O)c1cnn2cc(-c3cnn(C)c3)sc12]

To a solution of N-(5-(3-chloropropanamido)-2-methylpyridin-3-yl)-2-(1-methyl-1H-pyrazol-4-yl)pyrazolo[5,1-b]thiazole-7-carboxamide (120 mg, 0.14 mmol) and 6-azaspiro[2.5]octane (61 mg, 0.55 mmol) in DMSO (4 mL) was added DBU (31 mg, 0.21 mmol). The resulting mixture was stirred at 60° C. for 1 h before cooled to 25° C. The reaction mixture was diluted with ethyl acetate (30 mL) and filtered over celite. The filtrate was concentrated to the crude product, which was treated with ethyl acetate (90 mL) and water (30 mL). The organic phase was washed with 3 M HCl aqueous solution, dried over anhydrous Na₂SO₄ and filtered. The filtrate was concentrated under reduced pressure to give the crude product, which was purified by preparative high-performance liquid chromatography over column: Boston Green ODS 150*30 mm*5 um to give the title compound N-(5-(3-(6-azaspiro[2.5]octan-6-yl)propanamido)-2-methylpyridin-3-yl)-2-(1-methyl-1H-pyrazol-4-yl)pyrazolo[5,1-b]thiazole-7-carboxamide (50 mg, 64%) as a white solid. LCMS (ESI): mass calcd. for C₂₆H₃₀N₈O₂S, 518.634; m/z found, 519.3 [M+H]⁺. ¹H NMR (400 MHz, METHANOL-d₄) δ 8.59 (d, J=2.3 Hz, 1H), 8.41 (s, 1H), 8.29-8.23 (m, 2H), 8.05 (s, 1H), 7.84 (s, 1H), 3.96 (s, 3H), 3.36 (br s, 2H), 3.17 (br s, 4H), 2.90 (t, J=6.8 Hz, 2H), 2.52 (s, 3H), 1.68 (br s, 4H), 0.48 (s, 4H).

### Example 98: N-(5-(3-(2-azabicyclo[2.2.2]octan-2-yl)propanamido)-2-methylpyridin-3-yl)-2-(1-(2-methoxyethyl)-1H-pyrazol-4-yl)pyrazolo[5,1-b]thiazole-7-carboxamide

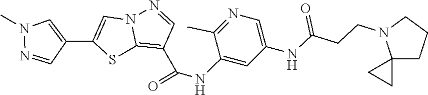 [SMILES: Cc1ncc(NC(=O)CCN2CCCC23CC3)cc1NC(=O)c1cnn2cc(-c3cnn(C)c3)sc12]

To a solution of N-(5-(3-chloropropanamido)-2-methylpyridin-3-yl)-2-(1-methyl-1H-pyrazol-4-yl)pyrazolo[5,1-b]thiazole-7-carboxamide (100 mg, 0.12 mmol) and 4-azaspiro[2.4]heptane (45 mg, 0.46 mmol) in DMSO (4 mL) was added DBU (26 mg, 0.17 mmol). The resulting mixture was stirred at 60° C. for 1 h before cooled to 25° C. The reaction mixture was diluted with ethyl acetate (30 mL) and filtered over celite. The filtrate was concentrated to the crude product, which was treated with ethyl acetate (90 mL) and water (30 mL). The organic phase was washed with 3 M HCl aqueous solution, dried over anhydrous Na₂SO₄ and filtered. The filtrate was concentrated under reduced pressure to give the crude product, which was purified by preparative high-performance liquid chromatography over column: Welch Xtimate C18 150*30 mm*5 um to give the title compound N-(5-(3-(2-azabicyclo[2.2.2]octan-2-yl)propanamido)-2-methylpyridin-3-yl)-2-(1-(2-methoxyethyl)-1H-pyrazol-4-yl)pyrazolo[5,1-b]thiazole-7-carboxamide (23 mg, 40%) as a white solid. LCMS (ESI): mass calcd. for C₂₅H₂₈N₈O₂S, 504.607; m/z found, 505.3 [M+H]⁺. ¹H NMR (400 MHz, METHANOL-d₄) δ 8.53 (d, J=2.2 Hz, 1H), 8.38 (s, 1H), 8.24-8.19 (m, 2H), 8.02 (s, 1H), 7.82 (s, 1H), 3.94 (s, 3H), 2.90 (t, J=6.9 Hz, 2H), 2.69-2.63 (m, 2H), 2.58-2.53 (m, 2H), 2.49 (s, 3H), 2.00-1.92 (m, 2H), 1.89-1.81 (m, 2H), 0.86-0.81 (m, 2H), 0.51-0.45 (m, 2H).

### Example 100: N-(5-(3-(2-azabicyclo[2.2.2]octan-2-yl)propanamido)-2-methylpyridin-3-yl)-2-(1-(2-methoxyethyl)-1H-pyrazol-4-yl)pyrazolo[5,1-b]thiazole-7-carboxamide

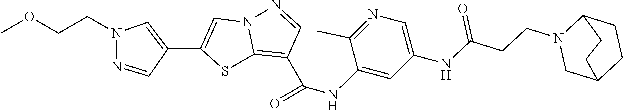 [SMILES: COCCn1cc(-c2cn3ncc(C(=O)Nc4cc(NC(=O)CCN5CC6CCC5CC6)cnc4C)c3s2)cn1]

To a solution of N-(5-(3-(2-azabicyclo[2.2.2]octan-2-yl)propanamido)-2-methylpyridin-3-yl)-2-bromopyrazolo[5,1-b]thiazole-7-carboxamide (200 mg, 0.39 mmol) and 1-(2-methoxyethyl)-4-(4,4,5,5-tetramethyl-1,3,2-dioxaborolan-2-yl)-1H-pyrazole (117 mg, 0.46 mmol) in 1,4-dioxane (4 mL) and H₂O (1 mL) was added [1,1-Bis(diphenylphosphino)ferrocene]palladium(II) chloride dichloromethane complex (32 mg, 0.039 mmol) and K₂CO₃ (107 mg, 0.77 mmol) under N₂. The resulting mixture was stirred at 90° C. under N₂ for 16 h before cooled to 25° C. The reaction mixture was diluted with ethyl acetate (30 mL) and filtered over celite. The filtrate was concentrated to the crude product, which was treated with ethyl acetate (90 mL) and water (30 mL). The organic phase was washed with 3 M HCl aqueous solution, dried over anhydrous Na₂SO₄ and filtered. The filtrate was concentrated under reduced pressure to give the crude product, which was purified by preparative high-performance liquid chromatography over column: Welch Xtimate C18 150*30 mm*5 um to give the title compound N-(5-(3-(2-azabicyclo[2.2.2]octan-2-yl)propanamido)-2-methylpyridin-3-yl)-2-(1-(2-methoxyethyl)-1H-pyrazol-4-yl)pyrazolo[5,1-b]thiazole-7-carboxamide (49 mg, 21%) as a white solid. LCMS (ESI): mass calcd. for C₂₈H₃₄N₈O₃S, 562.686; m/z found, 563.3 [M+H]⁺. ¹H NMR (400 MHz, METHANOL-d₄) δ 8.53 (s, 1H), 8.38 (s, 1H), 8.25-8.20 (m, 2H), 8.06 (s, 1H), 7.85 (s, 1H), 4.35 (t, J=5.1 Hz, 2H), 3.77 (t, J=5.0 Hz, 2H), 3.34 (s, 3H), 2.93 (br t, J=6.8 Hz, 2H), 2.80 (s, 2H), 2.69 (br s, 1H), 2.57 (t, J=7.0 Hz, 2H), 2.49 (s, 3H), 2.01 (br d, J=10.3 Hz, 2H), 1.72-1.53 (m, 7H).

### Example 102: N-(5-(3-(8-oxa-3-azabicyclo[3.2.1]octan-3-yl)propanamido)-2-methylpyridin-3-yl)-2-(1-(2-methoxyethyl)-3,5-dimethyl-1H-pyrazol-4-yl)pyrazolo[5,1-b]thiazole-7-carboxamide

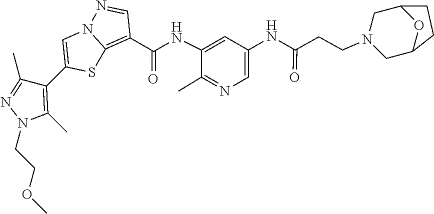 [SMILES: COCCn1nc(C)c(-c2cn3ncc(C(=O)Nc4cc(NC(=O)CCN5CC6CCC(C5)O6)cnc4C)c3s2)c1C]

### Step a: 1-(2-methoxyethyl)-3,5-dimethyl-4-(4,4,5,5-tetramethyl-1,3,2-dioxaborolan-2-yl)-1H-pyrazole

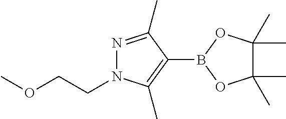

To a solution of 3,5-dimethyl-4-(4,4,5,5-tetramethyl-1,3,2-dioxaborolan-2-yl)-1H-pyrazole (5 g, 22.5 mmol) in DMF (100 mL) was added 1-bromo-2-methoxyethane (6.26 g, 45 mmoL) and Cs₂CO₃ (15.5 g, 47 mmol) at room temperature. The reaction was stirred at 90° C. for 15 hrs. The reaction mixture was filtered. The filtrate was concentrated in vacuo to give brown oil. The brown oil was purified by column chromatography over silica gel (eluent: petroleum ether:ethyl acetate=9:1) to give the title compound 1-(2-methoxyethyl)-3,5-dimethyl-4-(4,4,5,5-tetramethyl-1,3,2-dioxaborolan-2-yl)-1H-pyrazole (5.8 g, 92%) as a colorless oil. LCMS (ESI): mass calcd. for C₁₄H₂₅BN₂O₃, 280.2; m/z found, 281.1 [M+H]⁺. ¹HNMR (400 MHz, CDCl₃) δ 4.06 (t, J=5.7 Hz, 2H), 3.63 (t, J=5.7 Hz, 2H), 3.23 (s, 3H), 2.34 (s, 3H), 2.26 (s, 3H), 1.22 (s, 12H).

### Step b: tert-butyl (5-(2-(1-(2-methoxyethyl)-3,5-dimethyl-1H-pyrazol-4-yl)pyrazolo[5,1-b]thiazole-7-carboxamido)-6-methylpyridin-3-yl)carbamate

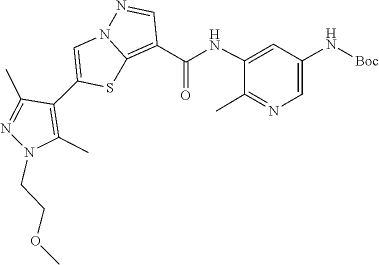 [SMILES: COCCn1nc(C)c(-c2cn3ncc(C(=O)Nc4cc(NC(=O)OC(C)(C)C)cnc4C)c3s2)c1C]

To a solution of tert-butyl (5-(2-bromopyrazolo[5,1-b]thiazole-7-carboxamido)-6-methylpyridin-3-yl)carbamate (2.3 g, 4.4 mmol), 1-(2-methoxyethyl)-3,5-dimethyl-4-(4,4,5,5-tetramethyl-1,3,2-dioxaborolan-2-yl)-1H-pyrazole (1.6 g, 5.8 mmol), Pd(dppf)Cl₂·CH₂Cl₂ (1.1 g, 1.3 mmol) in mixture of THF/H₂O (4:1, 15 mL) was added Cs₂CO₃ (4.4 mg, 14 mmol) at room temperature under N₂ atmosphere. The mixture was purged with N₂ with 2 minutes. Then the reaction mixture was stirred at 100° C. for 16 hours. After cooling down room temperature, the mixture was filtered off and the filtrate was evaporated to give crude product, which was purified by column chromatography over silica gel (eluent: dichloromethane:methanol=9:1) to give the title compound tert-butyl (5-(2-(1-(2-methoxyethyl)-3,5-dimethyl-1H-pyrazol-4-yl)pyrazolo[5,1-b]thiazole-7-carboxamido)-6-methylpyridin-3-yl)carbamate (1 g, 36%) as a pale brown solid. LCMS (ESI): mass calcd. for C₂₅H₃₁N₇O₄S, 525.2; m/z found, 526.4 [M+H]⁺.

### Step c: N-(5-amino-2-methylpyridin-3-yl)-2-(1-(2-methoxyethyl)-3,5-dimethyl-1H-pyrazol-4-yl)pyrazolo[5,1-b]thiazole-7-carboxamide

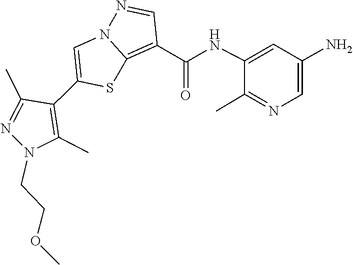 [SMILES: COCCn1nc(C)c(-c2cn3ncc(C(=O)Nc4cc(N)cnc4C)c3s2)c1C]

To a solution of tert-butyl (5-(2-(1-(2-methoxyethyl)-3,5-dimethyl-1H-pyrazol-4-yl)pyrazolo[5,1-b]thiazole-7-carboxamido)-6-methylpyridin-3-yl)carbamate (0.90 g, 1.5 mmol) in DCM (10 mL) was added HCl/dioxane (3.6 mL, 14 mmol, 4M) at room temperature. The reaction mixture was stirred at room temperature for 16 hours. The reaction mixture was concentrated in vacuo to give N-(5-amino-2-methylpyridin-3-yl)-2-(1-(2-methoxyethyl)-3,5-dimethyl-1H-pyrazol-4-yl)pyrazolo[5,1-b]thiazole-7-carboxamide (900 mg, crude) as a brown solid which was used in the next step without further work up and purification. LCMS (ESI): mass calcd. for C₂₀H₂₃N₇O₂S, 425.2; m/z found, 426.3 [M+H]⁺.

### Step d: N-(5-(3-chloropropanamido)-2-methylpyridin-3-yl)-2-(1-(2-methoxyethyl)-3,5-dimethyl-1H-pyrazol-4-yl)pyrazolo[5,1-b]thiazole-7-carboxamide

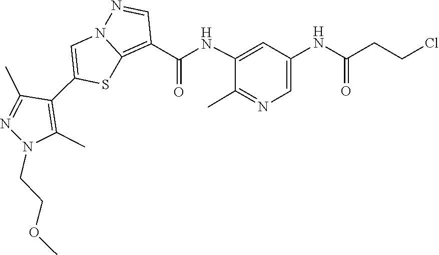 [SMILES: COCCn1nc(C)c(-c2cn3ncc(C(=O)Nc4cc(NC(=O)CCCl)cnc4C)c3s2)c1C]

To the mixture of N-(5-amino-2-methylpyridin-3-yl)-2-(1-(2-methoxyethyl)-3,5-dimethyl-1H-pyrazol-4-yl)pyrazolo[5,1-b]thiazole-7-carboxamide (900 mg, 1.7 mmol) in CHCl₃ (100 mL) and H₂O (100 mL) was added NaHCO₃ (7.0 g, 83 mmol) and 3-chloropropanoyl chloride (3 mL, 31 mmol) dropwise via syringe at room temperature over 5 min. The flask was sealed with a rubber septum. The mixture was stirred at room temperature for 1 hour. The reaction mixture was quenched by water (300 mL), and the mixture was extracted with ethyl acetate (300 mL×4). The organic layer was dried over anhydrous Na₂SO₄, filtered, and concentrated to dryness in vacuo to give N-(5-(3-chloropropanamido)-2-methylpyridin-3-yl)-2-(1-(2-methoxyethyl)-3,5-dimethyl-1H-pyrazol-4-yl)pyrazolo[5,1-b]thiazole-7 carboxamide (290 mg, 33%) as a gray solid. LCMS (ESI): mass calcd. for C₂₃H₂₆ClN₇O₃S, 515.2; m/z found, 516.2 [M+H]⁺.

### Step e: N-(5-(3-(8-oxa-3-azabicyclo[3.2.1]octan-3-yl)propanamido)-2-methylpyridin-3-yl)-2-(1-(2-methoxyethyl)-3,5-dimethyl-1H-pyrazol-4-yl)pyrazolo[5,1-b]thiazole-7-carboxamide

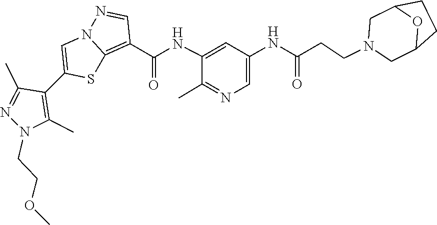 [SMILES: COCCn1nc(C)c(-c2cn3ncc(C(=O)Nc4cc(NC(=O)CCN5CC6CCC(C5)O6)cnc4C)c3s2)c1C]

To a mixture of N-(5-(3-chloropropanamido)-2-methylpyridin-3-yl)-2-(1-(2-methoxyethyl)-3,5-dimethyl-1H-pyrazol-4-yl)pyrazolo[5,1-b]thiazole-7-carboxamide (90 mg, 0.17 mmol), 8-oxa-3-azabicyclo[3.2.1]octane (80 mg, 0.71 mmol) in CH₃CN (7 mL) was added Et₃N (142 mg, 1.403 mmol) and at room temperature. The mixture was stirred at 70° C. for 15 hours. The mixture stirred at room temperature for 1 hour and then concentrated under vacuum to give the crude product, which was purified by preparative high-performance liquid chromatography over column: Phenomenex Gemini-NX C18 75*30 mm*3 um to give the title compound N-(5-(3-(8-oxa-3-azabicyclo[3.2.1]octan-3-yl)propanamido)-2-methylpyridin-3-yl)-2-(1-(2-methoxyethyl)-3,5-dimethyl-1H-pyrazol-4-yl)pyrazolo[5,1-b]thiazole-7-carboxamide (31.9 mg, 32%) as a gray solid. LCMS (ESI): mass calcd. for C₂₉H₃₆N₈O₄S, 592.3; m/z found, 593.3 [M+H]⁺. ¹H NMR (400 MHz, METHANOL-d₄) δ 8.58 (d, J=2.3 Hz, 1H), 8.44 (s, 1H), 8.25 (d, J=2.3 Hz, 1H), 8.04 (s, 1H), 4.33-4.21 (m, 4H), 3.74 (t, J=5.2 Hz, 2H), 3.34 (br s, 3H), 2.78-2.68 (m, 4H), 2.60-2.54 (m, 2H), 2.52 (s, 3H), 2.41 (s, 3H), 2.36 (s, 1H), 2.33 (s, 4H), 1.96-1.88 (m, 2H), 1.88-1.77 (m, 2H).

### Example 103: N-(5-(3-(5-azaspiro[2.4]heptan-5-yl)propanamido)-2-methylpyridin-3-yl)-2-(1-(2-methoxyethyl)-3,5-dimethyl-1H-pyrazol-4-yl)pyrazolo[5,1-b]thiazole-7-carboxamide

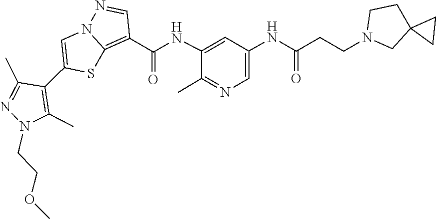 [SMILES: COCCn1nc(C)c(-c2cn3ncc(C(=O)Nc4cc(NC(=O)CCN5CCC6(CC6)C5)cnc4C)c3s2)c1C]

To a solution of N-(5-(3-chloropropanamido)-2-methylpyridin-3-yl)-2-(1-(2-methoxyethyl)-3,5-dimethyl-1H-pyrazol-4-yl)pyrazolo[5,1-b]thiazole-7-carboxamide (80 mg, 0.15 mmol) in MeCN (4 mL) was added 5-azaspiro[2.4]heptane hydrochloride (80 mg, 0.60 mmol) and TEA (0.17 mL, 1.2 mmol) at room temperature. The resulting mixture was stirred at 70° C. for 16 h before cooling to room temperature. The reaction mixture was filtered and the filtrate was concentrated under vacuum to give crude product, which was purified by preparative high-performance liquid chromatography over column: Phenomenex C18 80*40 mm*3 um to give the title compound N-(5-(3-(5-azaspiro[2.4]heptan-5-yl)propanamido)-2-methylpyridin-3-yl)-2-(1-(2-methoxyethyl)-3,5-dimethyl-1H-pyrazol-4-yl)pyrazolo[5,1-b]thiazole-7-carboxamide (23 mg, 26%) as a white solid. LCMS (ESI): mass calcd. for C₂₉H₃₆N₈O₃S 576.7; m/z found, 577.2 [M+H]⁺. ¹H NMR (400 MHz, METHANOL-d₄) δ 8.58 (d, J=2.38 Hz, 1H), 8.45 (s, 1H), 8.25 (d, J=2.27 Hz, 1H), 8.05 (s, 1H), 4.26 (t, J=5.19 Hz, 2H), 3.74 (t, J=5.13 Hz, 2H), 3.32 (br s, 3H), 3.05 (br d, J=6.08 Hz, 4H), 2.78 (br s, 2H), 2.71 (t, J=7.27 Hz, 2H), 2.52 (s, 3H), 2.41 (s, 3H), 2.33 (s, 3H), 1.94 (t, J=7.15 Hz, 2H), 0.60-0.70 (m, 4H).

### Example 104: N-(5-(3-(5-azaspiro[2.4]heptan-5-yl)propanamido)-2-methylpyridin-3-yl)-2-(1-methyl-1H-pyrazol-4-yl)pyrazolo[5,1-b]thiazole-7-carboxamide

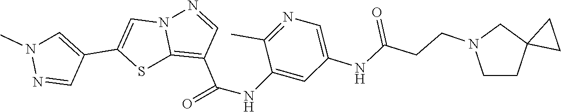 [SMILES: Cc1ncc(NC(=O)CCN2CCC3(CC3)C2)cc1NC(=O)c1cnn2cc(-c3cnn(C)c3)sc12]

### Step a: N-(5-(3-(5-azaspiro[2.4]heptan-5-yl)propanamido)-2-methylpyridin-3-yl)-2-bromopyrazolo[5,1-b]thiazole-7-carboxamide

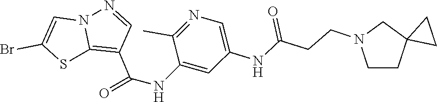 [SMILES: Cc1ncc(NC(=O)CCN2CCC3(CC3)C2)cc1NC(=O)c1cnn2cc(Br)sc12]

To a solution of 2-bromo-N-(5-(3-chloropropanamido)-2-methylpyridin-3-yl)pyrazolo[5,1-b]thiazole-7-carboxamide (400 mg, 0.90 mmol) and 5-azaspiro[2.4]heptane (176 mg, 1.8 mmol) in DMF (5 mL) was added K₂CO₃ (375 mg, 2.7 mmol) and KI (15 mg, 0.09 mmol) at room temperature. The mixture was stirred at 60° C. for 1.5 h. The resulting mixture was quenched with water (5 mL) and extracted with ethyl acetate (10 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=100:0-0:100) to give the title compound: N-(5-(3-(5-azaspiro[2.4]heptan-5-yl)propanamido)-2-methylpyridin-3-yl)-2-bromopyrazolo[5,1-b]thiazole-7-carboxamide (200 mg, 44%) as a yellow solid. LCMS (ESI): mass calcd. for C₂₁H₂₃BrN₆O₂S, 503.4; m/z found, 504.1[M+H]⁺.

### Step b: N-(5-(3-(5-azaspiro[2.4]heptan-5-yl)propanamido)-2-methylpyridin-3-yl)-2-(1-methyl-1H-pyrazol-4-yl)pyrazolo[5,1-b]thiazole-7-carboxamide

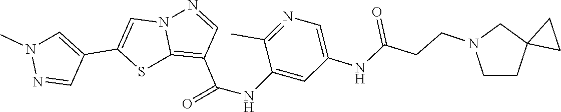 [SMILES: Cc1ncc(NC(=O)CCN2CCC3(CC3)C2)cc1NC(=O)c1cnn2cc(-c3cnn(C)c3)sc12]

To a solution of N-(5-(3-(5-azaspiro[2.4]heptan-5-yl)propanamido)-2-methylpyridin-3-yl)-2-bromopyrazolo[5,1-b]thiazole-7-carboxamide (150 mg, 0.30 mmol), 1-methyl-4-(4,4,5,5-tetramethyl-1,3,2-dioxaborolan-2-yl)-1H-pyrazole (74 mg, 0.36 mmol) and K₂CO₃ (82 mg, 0.60 mmol) in dioxane (4 mL) and H₂O (1 mL) was added Pd(dppf)Cl₂·CH₂Cl₂ (24 mg, 0.03 mmol) at room temperature. The resulting mixture was stirred at 90° C. overnight before cooling to room temperature. The reaction mixture was filtered through a pad of celite and the filtrate was concentrated under reduced pressure to give crude product, which was purified by preparative high-performance liquid chromatography over column: Welch Xtimate C18 150*30 mm*5 um to give the title compound N-(5-(3-(5-azaspiro[2.4]heptan-5-yl)propanamido)-2-methylpyridin-3-yl)-2-(1-methyl-1H-pyrazol-4-yl)pyrazolo[5,1-b]thiazole-7-carboxamide (41 mg, 26%) as a white solid. LCMS (ESI): mass calcd. for C₂₅H₂₈N₈O₂S, 504.6; m/z found, 505.3 [M+H]⁺. ¹H NMR (400 MHz, METHANOL-d₄) δ 8.55 (d, J=2.3 Hz, 1H), 8.38 (s, 1H), 8.26-8.16 (m, 2H), 8.03 (s, 1H), 7.83 (s, 1H), 3.94 (s, 3H), 2.87 (td, J=7.2, 19.3 Hz, 4H), 2.66-2.57 (m, 4H), 2.49 (s, 3H), 1.87 (t, J=7.0 Hz, 2H), 0.63-0.55 (m, 4H).

### Example 106: (S)-2-(1-methyl-1H-pyrazol-4-yl)-N-(2-methyl-5-(4-(2-methylpyrrolidin-1-yl)butanamido)pyridin-3-yl)pyrazolo[5,1-b]thiazole-7-carboxamide

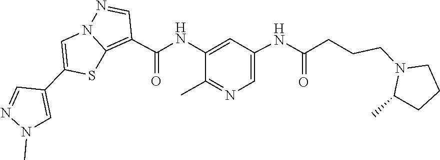 [SMILES: Cc1ncc(NC(=O)CCCN2CCC[C@@H]2C)cc1NC(=O)c1cnn2cc(-c3cnn(C)c3)sc12]

### Step a: N-(5-(4-chlorobutanamido)-2-methylpyridin-3-yl)-2-(1-methyl-1H-pyrazol-4-yl)pyrazolo[5,1-b]thiazole-7-carboxamide

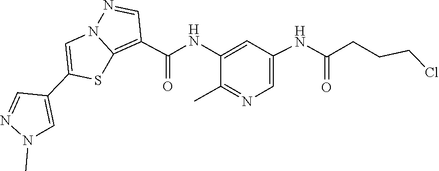 [SMILES: Cc1ncc(NC(=O)CCCCl)cc1NC(=O)c1cnn2cc(-c3cnn(C)c3)sc12]

To a mixture of N-(5-amino-2-methylpyridin-3-yl)-2-(1-methyl-1H-pyrazol-4-yl)pyrazolo[5,1-b]thiazole-7-carboxamide hydrochloride (650 mg, 1.5 mmol) and NaHCO₃ (640 mg, 7.6 mmol) in DMF (13 mL) was added 4-chlorobutanoyl chloride (0.27 mL, 2.3 mmoL) was added dropwise at 0° C. The reaction was warmed to room temperature and stirred for 1.5 hours. The reaction mixture was filtered. The filtrate was concentrated in vacuo to give brown solid, which was purified by column chromatography over silica gel (eluent: dichloromethane:methanol=9:1) to give the title compound N-(5-(4-chlorobutanamido)-2-methylpyridin-3-yl)-2-(1-methyl-1H-pyrazol-4-yl)pyrazolo[5,1-b]thiazole-7-carboxamide (530 mg, 76%) as a colorless oil. LCMS (ESI): mass calcd. for C₂₀H₂OClN₇O₂S, 457.1; m/z found, 458.1 [M+H]⁺.

### Step b: (S)-2-(1-methyl-1H-pyrazol-4-yl)-N-(2-methyl-5-(4-(2-methylpyrrolidin-1-yl)butanamido)pyridin-3-yl)pyrazolo[5,1-b]thiazole-7-carboxamide

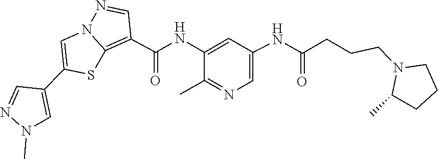 [SMILES: Cc1ncc(NC(=O)CCCN2CCC[C@@H]2C)cc1NC(=O)c1cnn2cc(-c3cnn(C)c3)sc12]

A mixture of N-(5-(4-chlorobutanamido)-2-methylpyridin-3-yl)-2-(1-methyl-1H-pyrazol-4-yl)pyrazolo[5,1-b]thiazole-7-carboxamide (100 mg, 0.22 mmol) in (S)-2-methylpyrrolidine (2 mL) was stirred at 60° C. for 15 hours. The reaction mixture was concentrated in vacuo to give brown solid, which was purified by preparative high-performance liquid chromatography over column: Phenomenex Gemini-NX C18 75*30 mm*3 um to give the title compound (S)-2-(1-methyl-1H-pyrazol-4-yl)-N-(2-methyl-5-(4-(2-methylpyrrolidin-1-yl)butanamido)pyridin-3-yl)pyrazolo[5,1-b]thiazole-7-carboxamide (Formic acid salt, 48 mg, 44%) as a gray solid. LCMS (ESI): mass calcd. for C₂₅H₃₀N₈O₂S, 506.2; m/z found, 507.2 [M+H]⁺. ¹H NMR (400 MHz, METHANOL-d₄) δ 8.58 (d, J=2.3 Hz, 1H), 8.56 (br s, 1H), 8.28-8.24 (m, 2H), 8.05 (s, 1H), 7.84 (s, 1H), 3.97 (s, 3H), 3.69-3.57 (m, 1H), 3.39-3.37 (m, 2H), 3.18-3.05 (m, 1H), 3.02-2.91 (m, 1H), 2.61 (t, J=6.9 Hz, 2H), 2.52 (s, 3H), 2.35-2.23 (m, 1H), 2.18-1.98 (m, 4H), 1.79-1.65 (m, 1H), 1.42 (d, J=6.6 Hz, 3H).

### Example 107: 2-(1-methyl-1H-pyrazol-4-yl)-N-(2-methyl-5-(3-((1-methylpiperidin-4-yl)methyl)ureido)pyridin-3-yl)pyrazolo[5,1-b]thiazole-7-carboxamide

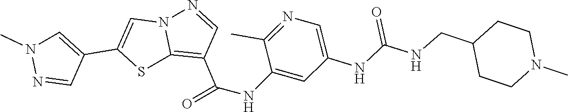

### Step a: 2-amino-3,5-dinitro-6-methylpyridine

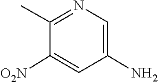

To a solution of tert-butyl (6-methyl-5-nitropyridin-3-yl) carbamate (5 g, 19.7 mmol) in DCM (100 mL) was added HCl in MeOH (4 M, 138.5 mL). The mixture reaction was stirred at 60° C. for 1 day. TLC showed the starting material was consumed completely. The resulting mount of solid was found. The mixture was concentrated dryness and tert-butyl methyl ether (200 mL) was added. The solid was collected, washed with tert-butyl methyl ether (50 mL) and dried under vacuum to give P1 as yellow solid. The yellow solid was dissolved in water (20 mL), NH₃·H₂O (3 mL) was added. Then extracted with ethyl acetate (3*100 mL). The organic layers were separated, dried with Na₂SO₄, and the solvent was evaporated to give 2-amino-3,5-dinitro-6-methylpyridine (1.938 g, 64%) as a yellow solid. ¹H NMR (400 MHz, CHLOROFORM-d) δ 8.14 (d, J=2.4 Hz, 1H), 7.50 (d, J=2.6 Hz, 1H), 4.00-3.71 (m, 2H), 2.66 (s, 3H)

### Step b: 4-nitrophenyl ((1-methylpiperidin-4-yl)methyl)carbamate

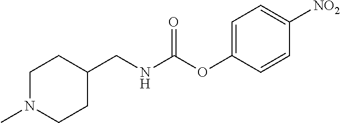 [SMILES: CN1CCC(CNC(=O)Oc2ccc([N+](=O)[O-])cc2)CC1]

To a solution of (1-methylpiperidin-4-yl)methanamine (750 mg, 5.85 mmol) in DCM (20 mL) was added 4-nitrophenyl chloroformate (1.18 g, 5.85 mmol), then Et₃N added at 0° C. The reaction mixture was stirred at 25° C. the desired product mass signal was found. The reaction mixture was partitioned between dichloromethane (3*10 mL) and water (5 mL). The organic layer was dried anhydrous sodium sulfate and evaporated to give the crude product as red oil. Evaporate the solution on a water bath under reduced pressure using a rotary evaporator to give compound 4-nitrophenyl ((1-methylpiperidin-4-yl)methyl)carbamate (1.5 g, 96%) a brown solid. LCMS (ESI): mass for C₁₄H₁₉N₃O₄: 293.32; m/z found: 293.9 [M+H]⁺.

### Step c: 1-(6-methyl-5-nitropyridin-3-yl)-3-((1-methylpiperidin-4-yl)methyl)urea

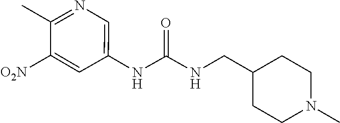 [SMILES: Cc1ncc(NC(=O)NCC2CCN(C)CC2)cc1[N+](=O)[O-]]

To the mixture of compound 4-nitrophenyl ((1-methylpiperidin-4-yl)methyl)carbamate (1.5 g, 4.953 mmol) in CAN (10 mL) was added 2-amino-3,5-dinitro-6-methylpyridine (0.756 g, 4.935 mmol), then DMAP (1.507 g, 12.337 mmol) added at 25° C. The mixture reaction was warmed to 80° C. for 12 hours. LCMS showed the starting material was consumed completely, the resulting mixture was evaporated un-der vacuum to give crude product as yellow oil. The product was purified with high-performance liquid chromatography over column: Xtimate C18 150*40 mm*5 um. The pure fractions were collected and the solvent was evaporated to give compound 1-(6-methyl-5-nitropyridin-3-yl)-3-((1-methylpiperidin-4-yl)methyl)urea (470 mg, 35%) as red oil. ¹H NMR (400 MHz, METHANOL-d₄) δ 8.61 (d, J=2.4 Hz, 1H), 8.60-8.57 (m, 1H), 3.10 (d, J=6.6 Hz, 2H), 2.88 (br d, J=11.7 Hz, 2H), 2.68 (s, 3H), 2.25 (s, 3H), 2.08-1.94 (m, 2H), 1.74 (br d, J=13.0 Hz, 2H), 1.59-1.45 (m, 1H), 1.28 (dq, J=3.5, 12.3 Hz, 2H)

### Step d: 1-(5-amino-6-methylpyridin-3-yl)-3-((1-methylpiperidin-4-yl)methyl)urea

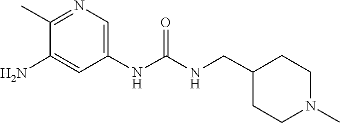

To a solution of compound 1-(6-methyl-5-nitropyridin-3-yl)-3-((1-methylpiperidin-4-yl)methyl)urea (470 mg, 1.529 mmol) in MeOH (10 mL) was hydrogenated at 25° C. (atmospheric pressure) with Pt/C (65.1 mg, 0.061 mmol) as a catalyst in the presence of H₂ for 12 hours. After uptake of H₂ (3 equivalent), the catalyst filtered off and the filtrate was evaporated give 1-(5-amino-6-methylpyridin-3-yl)-3-((1-methylpiperidin-4-yl)methyl)urea (400 mg, 80%) as yellow oil. LCMS (ESI): mass for C₁₄H₂₃N₅O: 277.4; m/z found: 278.2 [M+H]⁺. ¹H NMR (400 MHz, METHANOL-d₄) δ 7.67 (d, J=2.2 Hz, 1H), 7.23 (d, J=2.2 Hz, 1H), 3.07 (d, J=6.6 Hz, 2H), 2.91-2.82 (m, 2H), 2.25 (s, 3H), 2.24 (s, 3H), 2.05-1.93 (m, 2H), 1.72 (br d, J=12.8 Hz, 2H), 1.56-1.41 (m, 1H), 1.35-1.18 (m, 2H).

### Step e: 2-bromo-N-(2-methyl-5-(3-((1-methylpiperidin-4-yl)methyl)ureido)pyridin-3-yl)pyrazolo[5,1-b]thiazole-7-carboxamide

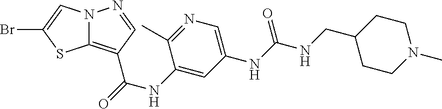 [SMILES: Cc1ncc(NC(=O)NCC2CCN(C)CC2)cc1NC(=O)c1cnn2cc(Br)sc12]

To a solution of 1-(5-amino-6-methylpyridin-3-yl)-3-((1-methylpiperidin-4-yl)methyl)urea (258 mg, 0.93 mmol) in SOCl₂ (5 mL) was added into the flask. The reaction mixture was stirred at 90° C. for 2 hours. Then solvent was evaporated to give the residue. To the mixture of 2-bromopyrazolo[5,1-b]thiazole-7-carboxylic acid (276 mg, 1.12 mmol) in pyridine (5 mL) was added the residue. The reaction mixture was stirred at 25° C. for 12 hours. The mixture was evaporated under vacuum to give the product. The crude product was purified by high-performance liquid chromatography over Xtimate C18 100*30 mm*3 um. The pure fractions were collected, and the solvent was evaporated to give the product as a yellow solid. The yellow solid was dissolved in water and acetonitrile, then dried by lyophilized to give 2-bromo-N-(2-methyl-5-(3-((1-methylpiperidin-4-yl)methyl)ureido)pyridin-3-yl)pyrazolo[5,1-b]thiazole-7-carboxamide (210 mg, 80%) as yellow solid. ¹H NMR (400 MHz, METHANOL-d₄) δ 8.42 (s, 1H), 8.36 (d, J=2.2 Hz, 1H), 8.31 (s, 1H), 8.01 (d, J=2.4 Hz, 1H), 3.46 (br s, 1H), 3.15 (br d, J=6.4 Hz, 2H), 2.97 (br s, 2H), 2.83 (s, 3H), 2.43 (s, 3H), 1.98 (br d, J=13.5 Hz, 2H), 1.81 (br s, 1H), 1.48 (br s, 3H).

### Step f: 2-(1-methyl-1H-pyrazol-4-yl)-N-(2-methyl-5-(3-((1-methylpiperidin-4-yl)methyl)ureido)pyridin-3-yl)pyrazolo[5,1-b]thiazole-7-carboxamide

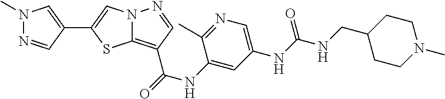

To a solution of 2-bromo-N-(2-methyl-5-(3-((1-methylpiperidin-4-yl)methyl)ureido)pyridin-3-yl)pyrazolo[5,1-b]thiazole-7-carboxamide (110 mg, 0.217 mmol) in the mixed solution of dioxane/H₂O (4:1.12 mL) was added 1-methyl-4-(3,3,4,4-tetramethylborolan-1-yl)-1H-pyrazole (67.8 mg, 0.326 mmol) and Cs₂CO₃ (375 mg, 1.15 mmol) at room temperature under N₂. Then dppf (35.5 mg, 0.04 mmol) was added and the mixture was stirred at 100° C. for 8 hours. The mixture was filtered through a celite pad, and the filter was evaporated under vacuum to give residue. The crude product was purified by high-performance liquid chromatography over Phenomenex Gemini-NX 80*40 mm*3 um. The pure fractions were collected, and the solvent was evaporated to give the desired product as a white solid. The yellow solid was dissolved in water and acetonitrile, then dried by lyophilized to give 2-(1-methyl-1H-pyrazol-4-yl)-N-(2-methyl-5-(3-((1-methylpiperidin-4-yl)methyl)ureido)pyridin-3-yl)pyrazolo[5,1-b]thiazole-7-carboxamide (25.9 mg, 39%) as a white solid. LCMS (ESI): mass for C₂₄H₂₉N₉O₂S: 507.6; m/z found: 508.2 [M+H]⁺. ¹H NMR (400 MHz, METHANOL-d₄) δ 8.40 (s, 1H), 8.38 (d, J=2.3 Hz, 1H), 8.26 (s, 1H), 8.07-8.03 (m, 2H), 7.85 (s, 1H), 3.97 (s, 3H), 3.14 (d, J=6.7 Hz, 2H), 2.98 (br d, J=12.3 Hz, 2H), 2.48 (s, 3H), 2.35 (s, 3H), 2.14 (br t, J=11.5 Hz, 2H), 1.80 (br d, J=13.0 Hz, 2H), 1.58 (br s, 1H), 1.42-1.25 (m, 2H).

### Example 108: 2-(1-methyl-1H-pyrazol-4-yl)-N-(2-methyl-5-(3-(2-(1-methylpiperidin-4-yl)ethyl)ureido)pyridin-3-yl)pyrazolo[5,1-b]thiazole-7-carboxamide

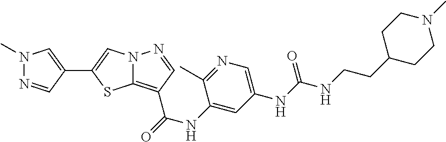

### Step a: 2-bromopyrazolo[5,1-b]thiazole-7-carboxylic acid

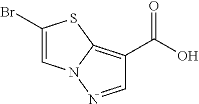 [SMILES: O=C(O)c1cnn2cc(Br)sc12]

To a solution of ethyl 2-bromopyrazolo[5,1-b]thiazole-7-carboxylate (3 g, 10.9 mmol) in EtOH (10 mL) was added aq.NaOH (3.751 mL, 2 mol/L). The mixture was stirred at 40° C. for 3 hours. Solvent was evaporated under reduced pressure. The mixture was adjusted to pH=3-4 with HCl (aq, 2 M). The mixture was filtered. The residue was washed with H₂O (10 mL×3). The solid was evaporated under vacuum to give 2-bromopyrazolo[5,1-b]thiazole-7-carboxylic acid (2.4 g, 96%) as white solid. LCMS (ESI): mass for C₆H₃BrN₂O₂S: 247.1; m/z found: 249.0 [M+H]⁺. ¹H NMR (400 MHz, DMSO-d₆) δ 12.84 (br s, 1H), 8.75 (s, 1H), 8.21 (s, 1H).

### Step b: N-(4-(((2-(1-methylpiperidin-4-yl)ethyl)carbamoyl)oxy)phenyl)-N-oxohydroxylammonium

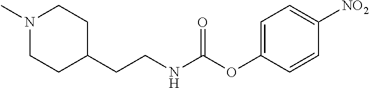 [SMILES: CN1CCC(CCNC(=O)Oc2ccc([N+](=O)[O-])cc2)CC1]

To a solution of 2-(1-methylpiperidin-4-yl)ethanamine (470 mg, 3.304 mmol) in DCM (15 mL) was added 4-nitrophenyl chloroformate (660 mg, 3.3 mmol), then Et₃N (600 mg, 6.6 mmol) added at 0° C. The reaction mixture was stirred at 25° C. the desired product mass signal was found. The reaction mixture was partitioned between dichloromethane (3*10 mL) and water (5 mL). The organic layer was dried anhydrous sodium sulfate and evaporated to give the crude product as red oil. Evaporate the solution on a water bath under reduced pressure using a rotary evaporator to give N-(4-(((2-(1-methylpiperidin-4-yl)ethyl)carbamoyl)oxy)phenyl)-N-oxohydroxylammonium (940 mg, 97%) a brown solid. LCMS (ESI): mass for C₁₅H₂₁N₃O₄: 307.3; m/z found: 308.2 [M+H]⁺.

### Step c: 1-(6-methyl-5-nitropyridin-3-yl)-3-(2-(1-methylpiperidin-4-yl)ethyl)urea

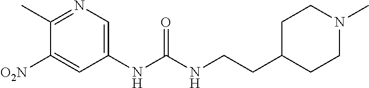

To a solution of N-(4-(((2-(1-methylpiperidin-4-yl)ethyl)carbamoyl)oxy)phenyl)-N-oxohydroxylammonium (940 mg, 2.98 mmol) in CAN (8 mL) was added 2-amino-3,5-dinitro-6-methylpyridine (457 mg, 2.98 mmol), then DMAP (910 mg, 7.46 mmol) added at 25° C. The mixture reaction was warmed to 80° C. for 12 hours. LCMS showed the starting material was consumed completely, the resulting mixture was evaporated un-der vacuum to give crude product as yellow oil. The product was purified with high-performance liquid chromatography over column: Xtimate C18 150*40 mm*5 um. The pure fractions were collected and the solvent was evaporated to give compound 1-(6-methyl-5-nitropyridin-3-yl)-3-(2-(1-methylpiperidin-4-yl)ethyl)urea (600 mg, 80%) as red oil. ¹H NMR (400 MHz, METHANOL-d₄) δ 8.66 (q, J=2.4 Hz, 2H), 3.47 (br d, J=12.4 Hz, 2H), 3.31-3.25 (m, 2H), 2.83 (s, 3H), 2.72 (s, 3H), 2.09-1.99 (m, 2H), 1.77-1.65 (m, 1H), 1.61-1.48 (m, 4H), 1.42-1.30 (m, 2H).

### Step d: 1-(5-amino-6-methylpyridin-3-yl)-3-(2-(1-methylpiperidin-4-yl)ethyl)urea

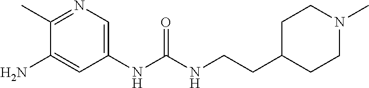

To a solution of compound 1-(6-methyl-5-nitropyridin-3-yl)-3-(2-(1-methylpiperidin-4-yl)ethyl)urea (600 mg, 1.867 mmol) in MeOH (10 mL) was hydrogenated at 25° C. (atmospheric pressure) with Pt/C (79.5 mg, 0.075 mmol) as a catalyst in the presence of H₂ for 12 hours. After uptake of H₂ (3 equivalent), the catalyst filtered off and the filtrate was evaporated give 1-(5-amino-6-methylpyridin-3-yl)-3-(2-(1-methylpiperidin-4-yl)ethyl)urea(200 mg, 95%) as yellow oil. LCMS (ESI): mass for C₁₅H₂₅N₅O: 291.4; m/z found: 292.2 [M+H]⁺.

### Step e: 2-bromo-N-(2-methyl-5-(3-(2-(1-methylpiperidin-4-yl)ethyl)ureido)pyridin-3-yl)pyrazolo[5,1-b]thiazole-7-carboxamide

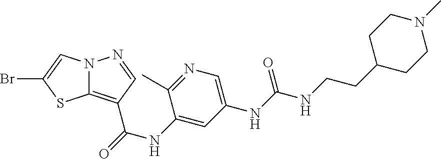 [SMILES: Cc1ncc(NC(=O)NCCC2CCN(C)CC2)cc1NC(=O)c1cnn2cc(Br)sc12]

Prepare a 25 mL thumb bottom flask with magnetic stirrer. The mixture of 2-bromopyrazolo[5,1-b]thiazole-7-carboxylic acid (336 mg, 1.3 mmol) in SOCl₂ (5 mL) was added into the flask. The reaction mixture was stirred at 90° C. for 2 hours. Then solvent was evaporated to give the residue. To the mixture of 1-(5-amino-6-methylpyridin-3-yl)-3-(2-(1-methylpiperidin-4-yl)ethyl)urea (190 mg, 0.652 mmol) in pyridine (5 mL) was added the residue. The reaction mixture was stirred at 25° C. for 12 hours. The mixture was evaporated under vacuum to give the product. The crude product was purified by high-performance liquid chromatography over Xtimate C18 100*30 mm*3 um. The pure fractions were collected, and the solvent was evaporated to give the product as a yellow solid. The yellow solid was dissolved in water and acetonitrile, then dried by lyophilized to give 2-bromo-N-(2-methyl-5-(3-(2-(1-methylpiperidin-4-yl)ethyl)ureido)pyridin-3-yl)pyrazolo[5,1-b]thiazole-7-carboxamide (220 mg, 31%) as yellow solid. LCMS (ESI): mass for C₂₁H₂₆BrN₇O₂S: 520.4; m/z found: 522.0 [M+H]⁺.

### Step f: 2-(1-methyl-1H-pyrazol-4-yl)-N-(2-methyl-5-(3-(2-(1-methylpiperidin-4-yl)ethyl)ureido)pyridin-3-yl)pyrazolo[5,1-b]thiazole-7-carboxamide

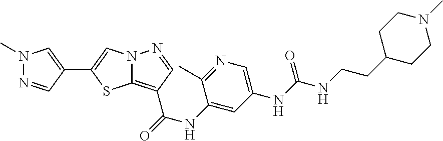

To a solution of 2-bromo-N-(2-methyl-5-(3-(2-(1-methylpiperidin-4-yl)ethyl)ureido)pyridin-3-yl)pyrazolo[5,1-b]thiazole-7-carboxamide (200 mg, 0.384 mmol) in the mixed solution of dioxane/H₂O (4:1, 15 mL) was added 1-methyl-4-(3,3,4,4-tetramethylborolan-1-yl)-1H-pyrazole (120 mg, 370 mmol) and Cs₂CO₃ (375 mg, 1.153 mmol) at room temperature under N₂. Then dppf (63 mg, 0.08 mmol) was added and the mixture was stirred at 100° C. for 8 hours. The mixture was filtered through a celite pad, and the filter was evaporated under vacuum to give residue. The crude product was purified by high-performance liquid chromatography over Phenomenex Gemini-NX 80*40 mm*3 um. The pure fractions were collected, and the solvent was evaporated to give the desired product as a white solid. The yellow solid was dissolved in water and acetonitrile, then dried by lyophilized to give 2-(1-methyl-1H-pyrazol-4-yl)-N-(2-methyl-5-(3-(2-(1-methylpiperidin-4-yl)ethyl)ureido)pyridin-3-yl)pyrazolo[5,1-b]thiazole-7-carboxamide (18 mg, 94%) as a white solid. LCMS (ESI): mass for C₂₅H₃₁N₉O₂S: 521.6; m/z found: 522.2 [M+H]⁺. ¹H NMR (400 MHz, METHANOL-d₄) δ 8.39-8.32 (m, 2H), 8.20 (s, 1H), 8.03-7.97 (m, 2H), 7.80 (s, 1H), 3.92 (s, 3H), 3.72-3.68 (m, 2H), 3.22 (brt, J=7.1 Hz, 2H), 2.84 (br d, J=11.5 Hz, 2H), 2.43 (s, 3H), 2.22 (s, 3H), 2.03-1.93 (m, 2H), 1.85 (td, J=3.4, 6.5 Hz, 2H), 1.50-1.43 (m, 1H), 1.28-1.20 (m, 2H).

### Example 109: N-(5-(3-((1-isopropylpiperidin-4-yl)methyl)ureido)-2-methylpyridin-3-yl)-2-(1-methyl-1H-pyrazol-4-yl)pyrazolo[5,1-b]thiazole-7-carboxamide

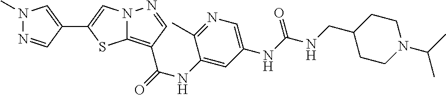 [SMILES: Cc1ncc(NC(=O)NCC2CCN(C(C)C)CC2)cc1NC(=O)c1cnn2cc(-c3cnn(C)c3)sc12]

### Step a: (1-isopropylpiperidin-4-yl)methanamine

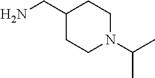

To a solution of tert-butyl ((1-isopropylpiperidin-4-yl)methyl)carbamate (3 g, 11.7 mmol) in DCM (15 mL) was added HCl in dioxane (15 mL, 4 M). The mixture reaction was stirred at 25° C. for 2 h. The mixture was concentrated dryness and tert-butyl methyl ether (200 mL) was added. The solid was collected, washed with tert-butyl methyl ether (50 mL) and dried under vacuum to give (1-isopropylpiperidin-4-yl)methanamine (3 g, 80%) as yellow solid. LCMS (ESI): mass for C₉H₂₀N₂: 156.3; m/z found: 157.2[M+H]⁺.

### Step b: phenyl (6-methyl-5-nitropyridin-3-yl)carbamate

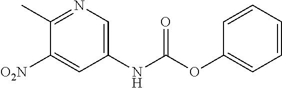 [SMILES: Cc1ncc(NC(=O)Oc2ccccc2)cc1[N+](=O)[O-]]

To a solution of compound (1-isopropylpiperidin-4-yl)methanamine (200 mg, 1.306 mmol) in THE (8 mL) was added phenyl carbonochloridate (245.8 μL, 1.96 mmol), then pyridine (210.2 μL, 2.6 mmol) added at 0° C. The mixture was stirred at 25° C. for 2 hours. The mixture was quenched with NH₄Cl (10 mL) and the mixture was extracted with ethyl acetate (15 mL*3). The combined organic layers were dried over Na₂SO₄, filtered, and concentrated in vacuum to give a crude product. The residue was purified by flash column chromatography over silica gel (eluent: petroleum ether/ethyl acetate from 100/0 to 70/30). The desired fractions were collected and the solvent was concentrated to dryness under vacuum to give compound phenyl (6-methyl-5-nitropyridin-3-yl)carbamate (350 mg, 85%) as a white solid. ¹H NMR (400 MHz, CHLOROFORM-d) δ 8.69 (s, 1H), 8.64 (br s, 1H), 7.45-7.36 (m, 2H), 7.30-7.21 (m, 2H), 7.18 (br d, J=8.2 Hz, 2H), 2.81 (s, 3H).

### Step c: 1-((1-isopropylpiperidin-4-yl)methyl)-3-(6-methyl-5-nitropyridin-3-yl)urea

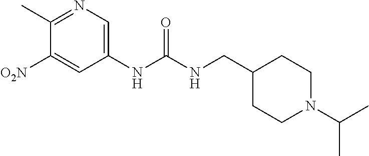 [SMILES: Cc1ncc(NC(=O)NCC2CCN(C(C)C)CC2)cc1[N+](=O)[O-]]

To the mixture of compound phenyl (6-methyl-5-nitropyridin-3-yl)carbamate (750 mg, 2.7 mmol) in CAN (35 mL) was added compound 2 (1.1 g, 4.12 mmol), then DMAP (670 mg, 5.49 mmol) added at 25° C. The mixture reaction was warmed to 80° C. for 12 hours. LCMS showed the starting material was consumed completely, the resulting mixture was evaporated under vacuum to give crude product as yellow oil. The product was purified with high-performance liquid chromatography over column: Phenomenex Genimi C18 150*40 mm*5 um. The pure fractions we-re collected and the solvent was evaporated to give compound 1-((1-isopropylpiperidin-4-yl)methyl)-3-(6-methyl-5-nitropyridin-3-yl)urea (550 mg, 80%) as yellow oil. ¹H NMR (400 MHz, METHANOL-d₄) δ 8.61 (br d, J=2.4 Hz, 1H), 8.60-8.53 (m, 1H), 3.33 (s, 1H), 3.10 (d, J=6.8 Hz, 2H), 2.92 (br d, J=11.7 Hz, 2H), 2.75-2.64 (m, 3H), 2.24-2.14 (m, 2H), 1.75 (br d, J=12.3 Hz, 2H), 1.58-1.45 (m, 1H), 1.34-1.20 (m, 2H), 1.07 (s, 3H), 1.05 (s, 3H).

### Step d: 1-(5-amino-6-methylpyridin-3-yl)-3-((1-isopropylpiperidin-4-yl)methyl)urea

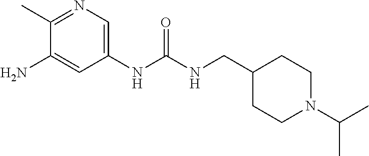 [SMILES: Cc1ncc(NC(=O)NCC2CCN(C(C)C)CC2)cc1N]

To the mixture of compound 1-((1-isopropylpiperidin-4-yl)methyl)-3-(6-methyl-5-nitropyridin-3-yl)urea (550 mg, 1.64 mmol) in MeOH (20 mL) was hydrogenated at 25° C. (atmospheric pressure) with Pt/C (70 mg, 0.07 mmol) as a catalyst in the presence of H₂ for 12 hours. After uptake of H₂ (3 equivalent), the catalyst filtered off and the filtrate was evaporated give 1-(5-amino-6-methylpyridin-3-yl)-3-((1-isopropylpiperidin-4-yl)methyl)urea (38 mg, 20%) as yellow solid. LCMS (ESI): mass for C₁₆H₂₇N₅O: 305.4; m/z found: 306.2 [M+H]⁺. ¹H NNMR (400 MHz, METHANOL-d₄) δ 7.67 (d, J=2.2 Hz, 1H), 7.23 (d, J=2.2 Hz, 1H), 3.07 (d, J=6.6 Hz, 2H), 2.93 (br d, J=11.2 Hz, 2H), 2.25 (s, 3H), 2.24-2.15 (m, 2H), 1.74 (br d, J=11.9 Hz, 2H), 1.56-1.42 (m, 1H), 1.27 (dq, J=3.5, 12.2 Hz, 2H), 1.07 (s, 3H), 1.05 (s, 3H).

### Step e: 2-bromo-N-(5-(3-((1-isopropylpiperidin-4-yl)methyl)ureido)-2-methylpyridin-3-yl)pyrazolo[5,1-b]thiazole-7-carboxamide

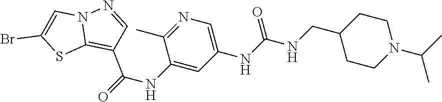 [SMILES: Cc1ncc(NC(=O)NCC2CCN(C(C)C)CC2)cc1NC(=O)c1cnn2cc(Br)sc12]

To a solution of 2-bromopyrazolo[5,1-b]thiazole-7-carboxylic acid (347 mg, 1.41 mmol) in SOCl₂ (3 mL) was added into the flask. The reaction mixture was stirred at 90° C. for 0.5 hours. Then solvent was evaporated to give the residue. To the mixture of 1-(5-amino-6-methylpyridin-3-yl)-3-((1-isopropylpiperidin-4-yl)methyl)urea (330 mg, 1.08 mmol) in pyridine (3 mL) was added the residue. The reaction mixture was stirred at 25° C. for 12 hours. The mixture was evaporated under vacuum to give the product. The crude product was purified by high-performance liquid chromatography over Boston Uni C18 40*150*5 um. The pure fractions were collected, and the solvent was evaporated to give the product as a yellow solid. The yellow solid was dissolved in water and acetonitrile, then dried by lyophilized to give 2-bromo-N-(5-(3-((1-isopropylpiperidin-4-yl)methyl)ureido)-2-methylpyridin-3-yl)pyrazolo[5,1-b]thiazole-7-carboxamide (300 mg, 99%) as yellow solid. LCMS (ESI): mass for C₂₂H₂₈BrN₇O₂S: 534.5; m/z found: 536.0 [M+H]⁺.

### Step f: N-(5-(3-((1-isopropylpiperidin-4-yl)methyl)ureido)-2-methylpyridin-3-yl)-2-(1-methyl-1H-pyrazol-4-yl)pyrazolo[5,1-b]thiazole-7-carboxamide

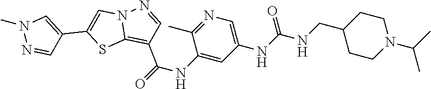 [SMILES: Cc1ncc(NC(=O)NCC2CCN(C(C)C)CC2)cc1NC(=O)c1cnn2cc(-c3cnn(C)c3)sc12]

To a solution of 2-bromo-N-(5-(3-((1-isopropylpiperidin-4-yl)methyl)ureido)-2-methylpyridin-3-yl)pyrazolo[5,1-b]thiazole-7-carboxamide (220 mg, 0.42 mmol) in the mixed solution of dioxane/H₂O (4:1, 45 mL) was added 1-methyl-4-(3,3,4,4-tetramethylborolan-1-yl)-1H-pyrazole (130 mg, 0.628 mmol) and Cs₂CO₃ (410 mg, 1.26 mmol) at room temperature under N₂. Then dppf (35 mg, 0.043 mmol) was added and the mixture was stirred at 100° C. for 8 hours. The mixture was filtered through a celite pad, and the filter was evaporated under vacuum to give residue. The crude product was purified by high-performance liquid chromatography over ASB Phenyl 150*30 mm*5 um. The pure fractions were collected, and the solvent was evaporated to give the desired product as a yellow solid. The yellow solid was dissolved in water and acetonitrile, then dried by lyophilized to give N-(5-(3-((1-isopropylpiperidin-4-yl)methyl)ureido)-2-methylpyridin-3-yl)-2-(1-methyl-1H-pyrazol-4-yl)pyrazolo[5,1-b]thiazole-7-carboxamide (29.2 mg, 100%) as a white solid. LCMS (ESI): mass for C₂₆H₃₃N₉O₂S: 535.7; m/z found: 536.2 [M+H]⁺. ¹H NMR (400 MHz, METHANOL-d₄) δ 8.42 (br d, J=2.2 Hz, 1H), 8.37 (s, 1H), 8.21 (s, 1H), 8.05 (br d, J=2.2 Hz, 1H), 8.01 (s, 1H), 7.80 (s, 1H), 3.92 (s, 3H), 3.46 (br d, J=7.1 Hz, 2H), 3.15 (br d, J=6.6 Hz, 2H), 3.01 (br t, J=11.9 Hz, 2H), 2.47 (s, 3H), 2.04 (br d, J=15.0 Hz, 2H), 1.83 (br s, 3H), 1.57-1.41 (m, 1H), 1.34 (s, 3H), 1.32 (s, 3H).

### Example 110: N-(5-((2-(7-oxa-4-azaspiro[2.5]octan-4-yl)ethyl)carbamoyl)-2-methylpyridin-3-yl)-2-(1-methyl-1H-pyrazol-4-yl)pyrazolo[5,1-b]thiazole-7-carboxamide

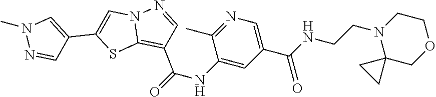 [SMILES: Cc1ncc(C(=O)NCCN2CCOCC23CC3)cc1NC(=O)c1cnn2cc(-c3cnn(C)c3)sc12]

### Step a: ethyl 5-(2-bromopyrazolo[5,1-b]thiazole-7-carboxamido)-6-methylnicotinate

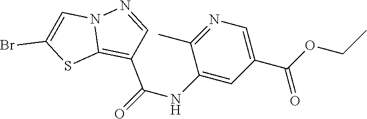 [SMILES: CCOC(=O)c1cnc(C)c(NC(=O)c2cnn3cc(Br)sc23)c1]

To a solution of 2-bromopyrazolo[5,1-b]thiazole-7-carboxylic acid (4.5 g, 18.11 mmol) in SOCl₂ (30 mL) was added into the flask. The reaction mixture was stirred at 90° C. for 1 hour. Then solvent was evaporated to give the residue. To the mixture of ethyl 5-amino-6-methylnicotinate (3.2 g, 18.11 mmol) in THF (150 mL) was added the residue, then TEA (10.1 mL, 72.45 mmol) added. The reaction mixture was stirred at 25° C. for 1 hour. The reaction mixture was concentrated under vacuum to give the crude product. The crude product was purified by column on silica gel (eluent: Petroleum ether/ethyl acetate=0% to 100%). The pure fractions were collected and the solvent was evaporated to give ethyl 5-(2-bromopyrazolo[5,1-b]thiazole-7-carboxamido)-6-methylnicotinate (3.15 g, 82%) as yellow solid. LCMS (ESI): mass for C₁₅H₁₃BrN₄O₃S: 409.3; m/z found: 409.0 [M+H]⁺

### Step b: ethyl 6-methyl-5-(2-(1-methyl-1H-pyrazol-4-yl)pyrazolo[5,1-b]thiazole-7-carboxamido)nicotinate

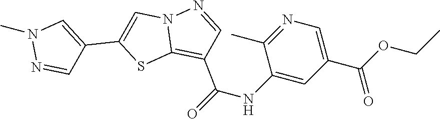 [SMILES: CCOC(=O)c1cnc(C)c(NC(=O)c2cnn3cc(-c4cnn(C)c4)sc23)c1]

To a solution of ethyl 5-(2-bromopyrazolo[5,1-b]thiazole-7-carboxamido)-6-methylnicotinate (3.15 g, 6.7 mmol) in the mixed solution of DMF/H₂O (4:1, 100 mL) was added 1-methyl-4-(4,4,5,5-tetramethyl-1,3,2-dioxaborolan-2-yl)-1H-pyrazole (1.68 g, 8.06 mmol) and Cs₂CO₃ (6.6 g, 20 mmol) at room temperature under N₂. Then dppf (1.097 g, 1.3 mmol) was added and the mixture was stirred at 95° C. for 13 hours. The reaction mixture was concentrated in vacuum to give a crude product. The crude product was adjusted pH=3-4 with HCl (aq, 2 M). The mixture was filtered. The residue was washed with H₂O (30 mL×3). The solid was evaporated under vacuum to give desired product as black solid. The black solid was purified by high-performance liquid chromatography over column: Boston Uni C18 40*150*5 um. The pure fractions were collected and the solvent was evaporated, then dried by lyophilized to give ethyl 6-methyl-5-(2-(1-methyl-1H-pyrazol-4-yl)pyrazolo[5,1-b]thiazole-7-carboxamido)nicotinate (90 mg, 99%) as a saddle solid. LCMS (ESI): mass for C₁₇H₁₄N₆O₃S: 382.4; m/z found: 383.0 [M+H]⁺

### Step c: 6-methyl-5-(2-(1-methyl-1H-pyrazol-4-yl)pyrazolo[5,1-b]thiazole-7-carboxamido)nicotinic acid

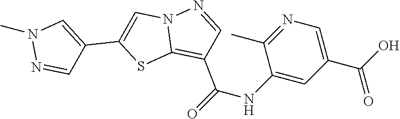 [SMILES: Cc1ncc(C(=O)O)cc1NC(=O)c1cnn2cc(-c3cnn(C)c3)sc12]

To a solution of ethyl 6-methyl-5-(2-(1-methyl-1H-pyrazol-4-yl)pyrazolo[5,1-b]thiazole-7-carboxamido)nicotinate (90 mg, 0.219 mmol) in mixture of THF:H₂O (1:1, 2 mL) was added LiOH (28 mg, 0.66 mmol). The mixture was stirred at room temperature for 2 h. The resultant was evaporated under vacuum, then adjusted to pH=3-4 with 1 mol/L HCl. The mixture was filtered under reduced pressure and the pad was washed with water (5 mL×3). The filter cake was evaporated under reduced pressure to give 6-methyl-5-(2-(1-methyl-1H-pyrazol-4-yl)pyrazolo[5,1-b]thiazole-7-carboxamido)nicotinic acid (90 mg, 90%) as a gray solid. LCMS (ESI): mass for C₁₇H₁₄N₆O₃S: 382.3; m/z found: 383.0 [M+H]+

### Step d: N-(5-((2-(7-oxa-4-azaspiro[2.5]octan-4-yl)ethyl)carbamoyl)-2-methylpyridin-3-yl)-2-(1-methyl-1H-pyrazol-4-yl)pyrazolo[5,1-b]thiazole-7-carboxamide

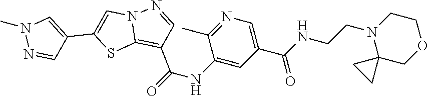 [SMILES: Cc1ncc(C(=O)NCCN2CCOCC23CC3)cc1NC(=O)c1cnn2cc(-c3cnn(C)c3)sc12]

To the mixture of 6-methyl-5-(2-(1-methyl-1H-pyrazol-4-yl)pyrazolo[5,1-b]thiazole-7-carboxamido)nicotinic acid (90 mg, 0.21 mmol) in DMF (3 mL) was added 2-(7-oxa-4-azaspiro[2.5]octan-4-yl)ethanamine (44 mg, 0.28 mmol) and DIEA (141 μL, 0.85 mmol), then HATU (121 mg, 0.32 mmol) was added. The resulting mixture was stirred at room temperature for 2 h. The reaction mixture was concentrated under vacuum to give product as brown oil. The mixture purified by column chromatography over 4 g silica gel (eluent: DCM/MeOH=100/0-70/30), and the fractions were collected and monitored by LCMS. The mixture was concentrated under vacuum to give crude product, which was purified by preparative high-performance liquid chromatography over column: Welch Xtimate C18 150*30 mm*5 um to give the final compound N-(5-((2-(7-oxa-4-azaspiro[2.5]octan-4-yl)ethyl)carbamoyl)-2-methylpyridin-3-yl)-2-(1-methyl-1H-pyrazol-4-yl)pyrazolo[5,1-b]thiazole-7-carboxamide (26.3 mg, 98%) as a white solid. LCMS (ESI): mass for C₂₅H₂₈N₈O₃S: 520.6; m/z found: 521.4 [M+H]⁺. ¹H NMR (400 MHz, METHANOL-d₄) δ 8.74 (d, J=2.2 Hz, 1H), 8.41 (s, 1H), 8.28 (d, J=2.0 Hz, 1H), 8.24 (s, 1H), 8.04 (s, 1H), 7.83 (s, 1H), 3.95 (s, 3H), 3.77-3.71 (m, 2H), 3.47 (s, 2H), 3.42 (t, J=6.7 Hz, 2H), 3.04-3.00 (m, 2H), 2.97 (t, J=6.8 Hz, 2H), 2.61 (s, 3H), 0.73-0.67 (m, 2H), 0.55-0.50 (m, 2H).

### Example 111: N-(5-((2-(4H-pyrrolo[3,4-d]thiazol-5 (6H)-yl)ethyl)carbamoyl)-2-methylpyridin-3-yl)-2-(1-methyl-1H-pyrazol-4-yl)pyrazolo[5,1-b]thiazole-7-carboxamide

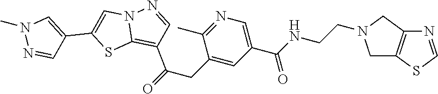 [SMILES: Cc1ncc(C(=O)NCCN2Cc3ncsc3C2)cc1CC(=O)c1cnn2cc(-c3cnn(C)c3)sc12]

### Step a: 2-(4H-pyrrolo[3,4-d]thiazol-5 (6H)-yl)acetonitrile

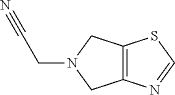 [SMILES: N#CCN1Cc2ncsc2C1]

To a solution of 5,6-dihydro-4H-pyrrolo[3,4-d]thiazole hydrochloride (500 mg, 3.1 mmol) and potassium carbonate (1.5 g, 11 mmol) in acetonitrile (8 mL) was added 2-bromoacetonitrile (406 mg, 3.4 mmol) at room temperature. The resulting mixture was stirred at 50° C. for 12 hours before cooling to room temperature. The resulting mixture was quenched with water (15 mL) and extracted with ethyl acetate (30 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=3:1) to give the title compound 2-(4H-pyrrolo[3,4-d]thiazol-5 (6H)-yl)acetonitrile (340 mg, 67%) as a pale yellow oil. ¹H NMR (400 MHz, CDCl₃) δ 8.76 (s, 1H), 4.28-4.22 (m, 2H), 4.21-4.16 (m, 1H), 4.21-4.16 (m, 1H), 3.87 (s, 2H).

### Step b: 2-(4H-pyrrolo[3,4-d]thiazol-5 (6H)-yl)ethanamine

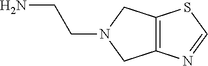 [SMILES: NCCN1Cc2ncsc2C1]

To a solution of 2-(4H-pyrrolo[3,4-d]thiazol-5 (6H)-yl)acetonitrile (300 mg, 1.8 mmol) in THE (6 mL) was added lithium aluminium hydride (103 mg, 2.7 mmol) by portions at 0° C. (ice/water). The resultant mixture was stirred at room temperature for 1 hour before quenched with water (0.6 mL) at 0° C. The reaction mixture was filtered. And the filtrate was concentrated to dryness under reduced pressure to afford the crude product 2-(4H-pyrrolo[3,4-d]thiazol-5 (6H)-yl)ethanamine (220 mg, 72%) as a colorless oil, which was used to the next step without further purification. ¹H NMR (400 MHz, CDCl₃) δ 8.63 (s, 1H), 4.03-3.99 (m, 2H), 3.96-3.92 (m, 2H), 2.89-2.79 (m, 4H).

### Step c: N-(5-((2-(4H-pyrrolo[3,4-d]thiazol-5 (6H)-yl)ethyl)carbamoyl)-2-methylpyridin-3-yl)-2-(1-methyl-1H-pyrazol-4-yl)pyrazolo[5,1-b]thiazole-7-carboxamide

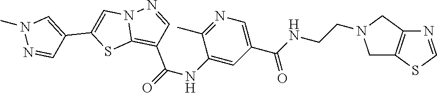 [SMILES: Cc1ncc(C(=O)NCCN2Cc3ncsc3C2)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (200 mg, 0.26 mmol), HATU (179 mg, 0.47 mmol) and N,N-diisopropylethylamine (0.18 mL, 1.0 mmol) in N,N-dimethylformamide (6 mL) was added 2-(4H-pyrrolo[3,4-d]thiazol-5 (6H)-yl)ethanamine (66 mg, 0.39 mmol). The mixture stirred at room temperature for 1 hour and then concentrated under vacuum to give the crude product, which was purified by preparative high-performance liquid chromatography over column: Welch Xtimate C18 150*25 mm*5 um to give the title compound N-(5-((2-(4H-pyrrolo[3,4-d]thiazol-5 (6H)-yl)ethyl)carbamoyl)-2-methylpyridin-3-yl)-2-(1-methyl-1H-pyrazol-4-yl)pyrazolo[5,1-b]thiazole-7-carboxamide (45 mg, 23%) as a gray solid. LCMS (ESI): mass calcd. for C₂₄H₂₃N₉O₂S₂, 533.1; m/z found, 534.2 [M+H]⁺. ¹H NMR (400 MHz, METHANOL-d₄) δ 8.93 (s, 1H), 8.82 (d, J=1.5 Hz, 1H), 8.42 (s, 1H), 8.34 (d, J=2.0 Hz, 1H), 8.26 (s, 1H), 8.06 (s, 1H), 7.85 (s, 1H), 4.18 (br s, 2H), 4.07 (br s, 2H), 3.96 (s, 3H), 3.66 (t, J=6.4 Hz, 2H), 3.13 (t, J=6.4 Hz, 2H), 2.62 (s, 3H).

### Example 112: 2-(1-methyl-1H-pyrazol-4-yl)-N-(2-methyl-5-((2-(tetrahydro-1H-furo[3,4-c]pyrrol-5 (3H)-yl)ethyl)carbamoyl)pyridin-3-yl)pyrazolo[5,1-b]thiazole-7-carboxamide

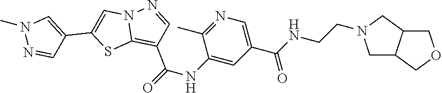 [SMILES: Cc1ncc(C(=O)NCCN2CC3COCC3C2)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (120 mg, 0.23 mmol), HATU (155 mg, 0.41 mmol) and N,N-diisopropylethylamine (0.16 mL, 0.91 mmol) in N,N-dimethylformamide (5 mL) was added 2-(tetrahydro-1H-furo[3,4-c]pyrrol-5 (3H)-yl)ethanamine (53 mg, 0.34 mmol). The mixture stirred at room temperature for 1 hour and then concentrated under vacuum to give the crude product, which was purified by preparative high-performance liquid chromatography over column: Welch Xtimate C18 150*25 mm*5 um to give the title compound 2-(1-methyl-1H-pyrazol-4-yl)-N-(2-methyl-5-((2-(tetrahydro-1H-furo[3,4-c]pyrrol-5 (3H)-yl)ethyl)carbamoyl)pyridin-3-yl)pyrazolo[5,1-b]thiazole-7-carboxamide (52 mg, 43%) as a gray solid. LCMS (ESI): mass calcd. for C₂₅H₂₈N₈O₃S, 520.2; m/z found, 521.3 [M+H]⁺. ¹H NMR (400 MHz, METHANOL-d₄) δ 8.80 (d, J=2.0 Hz, 1H), 8.42 (s, 1H), 8.32 (d, J=1.9 Hz, 1H), 8.26 (s, 1H), 8.06 (s, 1H), 7.85 (s, 1H), 3.97 (s, 3H), 3.74 (br dd, J=5.3, 9.0 Hz, 2H), 3.65 (br d, J=8.0 Hz, 2H), 3.57 (t, J=6.6 Hz, 2H), 2.91-2.84 (m, 4H), 2.70 (t, J=6.6 Hz, 2H), 2.63 (s, 3H), 2.40 (br d, J=5.2 Hz, 2H).

### Example 113: N-(5-((2-(6-azaspiro[3.4]octan-6-yl)ethyl)carbamoyl)-2-methylpyridin-3-yl)-2-(1-methyl-1H-pyrazol-4-yl)pyrazolo[5,1-b]thiazole-7-carboxamide

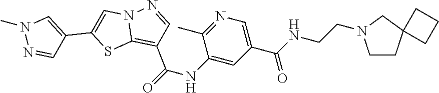 [SMILES: Cc1ncc(C(=O)NCCN2CCC3(CCC3)C2)cc1NC(=O)c1cnn2cc(-c3cnn(C)c3)sc12]

### Step a: 2-(6-azaspiro[3.4]octan-6-yl)acetonitrile

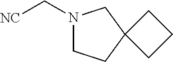 [SMILES: N#CCN1CCC2(CCC2)C1]

To a solution of 6-azaspiro[3.4]octane (350 mg, 3.1 mmol) and potassium carbonate (1.1 g, 7.9 mmol) in acetonitrile (7 mL) was added 2-bromoacetonitrile (415 mg, 3.5 mmol) at room temperature. The resulting mixture was stirred at 50° C. for 12 h before cooling to room temperature. The resulting mixture was quenched with water (15 mL) and extracted with ethyl acetate (30 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=3:1) to give the title compound 2-(6-azaspiro[3.4]octan-6-yl)acetonitrile (350 mg, 74%) as a pale yellow oil. ¹H NMR (400 MHz, CDCl₃) δ 3.53 (s, 2H), 2.66-2.58 (m, 4H), 1.98-1.70 (m, 8H).

### Step b: 2-(6-azaspiro[3.4]octan-6-yl)ethanamine

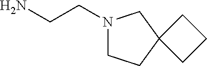 [SMILES: NCCN1CCC2(CCC2)C1]

To a solution of 2-(6-azaspiro[3.4]octan-6-yl)acetonitrile (300 mg, 2.0 mmol) in THE (6 mL) was added lithium aluminium hydride (114 mg, 3.0 mmol) by portions at 0° C. (ice/water). The resultant mixture was stirred at room temperature for 1 hour before quenched with water (0.6 mL) at 0° C. The reaction mixture was filtered. And the filtrate was concentrated to dryness under reduced pressure to afford the crude product 2-(6-azaspiro[3.4]octan-6-yl)ethanamine (250 mg, 81%) as a colorless oil, which was used to the next step without further purification. ¹H NMR (400 MHz, CDCl₃) δ 2.71 (t, J=6.4 Hz, 2H), 2.51 (s, 2H), 2.44 (td, J=6.7, 20.0 Hz, 4H), 1.99-1.67 (m, 10H).

### Step c: N-(5-((2-(6-azaspiro[3.4]octan-6-yl)ethyl)carbamoyl)-2-methylpyridin-3-yl)-2-(1-methyl-1H-pyrazol-4-yl)pyrazolo[5,1-b]thiazole-7-carboxamide

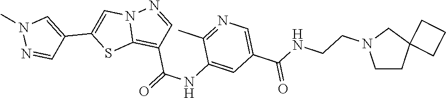 [SMILES: Cc1ncc(C(=O)NCCN2CCC3(CCC3)C2)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (200 mg, 0.26 mmol), HATU (179 mg, 0.47 mmol) and N,N-diisopropylethylamine (0.18 mL, 1.0 mmol) in N,N-dimethylformamide (6 mL) was added 2-(6-azaspiro[3.4]octan-6-yl)ethanamine (61 mg, 0.39 mmol). The mixture stirred at room temperature for 1 hour and then concentrated under vacuum to give the crude product, which was purified by preparative high-performance liquid chromatography over column: Phenomenex Gemini-NX C18 75*30 mm*3 um to give the title compound N-(5-((2-(6-azaspiro[3.4]octan-6-yl)ethyl)carbamoyl)-2-methylpyridin-3-yl)-2-(1-methyl-1H-pyrazol-4-yl)pyrazolo[5,1-b]thiazole-7-carboxamide (40 mg, 27%) as an off-white solid. LCMS (ESI): mass calcd. for C₂₆H₃₀N₈O₂S, 518.2; m/z found, 519.4 [M+H]⁺. ¹H NMR (400 MHz, METHANOL-d₄) δ 8.81 (d, J=2.0 Hz, 1H), 8.43 (s, 1H), 8.33 (d, J=1.9 Hz, 1H), 8.26 (s, 1H), 8.05 (s, 1H), 7.85 (s, 1H), 3.97 (s, 3H), 3.65-3.56 (m, 2H), 2.93-2.80 (m, 6H), 2.63 (s, 3H), 2.16-2.08 (m, 2H), 2.06-1.99 (m, 4H), 1.95-1.86 (m, 2H).

### Example 114: N-(5-((2-(2-oxa-5-azaspiro[3.4]octan-5-yl)ethyl)carbamoyl)-2-methylpyridin-3-yl)-2-(1-methyl-1H-pyrazol-4-yl)pyrazolo[5,1-b]thiazole-7-carboxamide

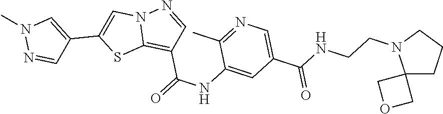 [SMILES: Cc1ncc(C(=O)NCCN2CCCC23COC3)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (80 mg, 0.21 mmol), HATU (143 mg, 0.38 mmol) and N,N-diisopropylethylamine (0.15 mL, 0.84 mmol) in N,N-dimethylformamide (4 mL) was added 2-(2-oxa-5-azaspiro[3.4]octan-5-yl)ethanamine (49 mg, 0.31 mmol). The mixture stirred at room temperature for 1 hour and then concentrated under vacuum to give the crude product, which was purified by preparative high-performance liquid chromatography over column: Phenomenex Gemini-NX 80*40 mm*3 um to give the title compound N-(5-((2-(2-oxa-5-azaspiro[3.4]octan-5-yl)ethyl)carbamoyl)-2-methylpyridin-3-yl)-2-(1-methyl-1H-pyrazol-4-yl)pyrazolo[5,1-b]thiazole-7-carboxamide (54 mg, 47%) as a pale brown solid. LCMS (ESI): mass calcd. for C₂₅H₂₈N₈O₃S, 520.2; m/z found, 521.3 [M+H]⁺. ¹H NMR (400 MHz, METHANOL-d₄) δ 8.79 (d, J=2.0 Hz, 1H), 8.43 (s, 1H), 8.32 (d, J=2.0 Hz, 1H), 8.26 (s, 1H), 8.06 (s, 1H), 7.85 (s, 1H), 4.83 (d, J=6.8 Hz, 2H), 4.53 (d, J=6.7 Hz, 2H), 3.97 (s, 3H), 3.61 (t, J=6.8 Hz, 2H), 3.15 (t, J=6.7 Hz, 2H), 2.88 (t, J=7.1 Hz, 2H), 2.63 (s, 3H), 2.21-2.15 (m, 2H), 1.80 (td, J=7.3, 14.9 Hz, 2H).

### Example 115: N-(5-((2-(cyclobutyl(2-methoxyethyl)amino)ethyl)carbamoyl)-2-methylpyridin-3-yl)-2-(1-methyl-1H-pyrazol-4-yl)pyrazolo[5,1-b]thiazole-7-carboxamide

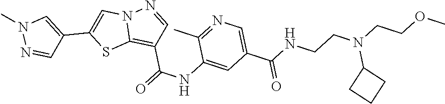

To a solution of 6-methyl-5-(2-(1-methyl-1H-pyrazol-4-yl)pyrazolo[5,1-b]thiazole-7-carboxamido)nicotinic acid (250 mg, 0.65 mmol), HATU (447 mg, 1.2 mmol) and N,N-diisopropylethylamine (0.34 mL, 2.0 mmol) in N,N-dimethylformamide (10 mL) was added N¹-cyclobutyl-N¹-(2-methoxyethyl)ethane-1,2-diamine (135 mg, 0.79 mmol). The mixture stirred at room temperature for 12 hours and then concentrated under vacuum to give the crude product, which was purified by preparative high-performance liquid chromatography over column: Phenomenex Gemini-NX C18 75*30 mm*3 um to give the title compound N-(5-((2-(cyclobutyl(2-methoxyethyl)amino)ethyl)carbamoyl)-2-methylpyridin-3-yl)-2-(1-methyl-1H-pyrazol-4-yl)pyrazolo[5,1-b]thiazole-7-carboxamide (Formic acid salt, 35 mg, 9%) as an off white solid. LCMS (ESI): mass calcd. for C₂₆H₃₂N₈O₃S, 536.2; m/z found, 537.5 [M+H]⁺. ¹H NMR (400 MHz, METHANOL-d₄) δ 8.80 (d, J=2.0 Hz, 1H), 8.46 (s, 1H), 8.35 (d, J=2.0 Hz, 1H), 8.27 (s, 1H), 8.06 (s, 1H), 7.85 (s, 1H), 3.97 (s, 3H), 3.65-3.56 (m, 5H), 3.38 (s, 3H), 3.02 (br s, 4H), 2.64 (s, 3H), 2.22 (br s, 2H), 2.11 (br d, J=9.5 Hz, 2H), 1.83-1.70 (m, 2H).

### Example 116: N-(5-((2-(2-oxa-7-azaspiro[4.4]nonan-7-yl)ethyl)carbamoyl)-2-methylpyridin-3-yl)-2-(1-methyl-1H-pyrazol-4-yl)pyrazolo[5,1-b]thiazole-7-carboxamide

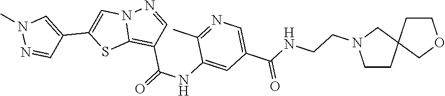 [SMILES: Cc1ncc(C(=O)NCCN2CCC3(CCOC3)C2)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (140 mg, 0.19 mmol), HATU (108 mg, 0.28 mmol) and N,N-diisopropylethylamine (0.13 mL, 0.76 mmol) in DMF (3 mL) was added 2-(2-oxa-7-azaspiro[4.4]nonan-7-yl)ethanamine (120 mg, 0.41 mmol). The resulting mixture was stirred at room temperature for 2 hours and then concentrated under vacuum to give the crude product, which was purified by preparative high-performance liquid chromatography over column: Xtimate C18 150*40 mm*5 um to give the title compound N-(5-((2-(2-oxa-7-azaspiro[4.4]nonan-7-yl)ethyl)carbamoyl)-2-methylpyridin-3-yl)-2-(1-methyl-1H-pyrazol-4-yl)pyrazolo[5,1-b]thiazole-7-carboxamide (48 mg, 46%) as a white solid. LCMS (ESI): mass calcd. for C₂₆H₃₀N₈O₃S, 534.2; m/z found, 535.4 [M+H]⁺. ¹H NMR (400 MHz, METHANOL-d₄) δ 8.79 (s, 1H), 8.42 (s, 1H), 8.33 (d, J=2.0 Hz, 1H), 8.25 (d, J=1.8 Hz, 1H), 8.08-8.02 (m, 1H), 7.87-7.81 (m, 1H), 3.96 (s, 3H), 3.89-3.84 (m, 2H), 3.69 (d, J=8.0 Hz, 1H), 3.58 (s, 2H), 2.83-2.71 (m, 6H), 2.62 (s, 3H), 2.07-1.86 (m, 5H).

### Example 117: N-(5-((2-(8-oxa-5-azaspiro[3.5]nonan-5-yl)ethyl)carbamoyl)-2-methylpyridin-3-yl)-2-(1-methyl-1H-pyrazol-4-yl)pyrazolo[5,1-b]thiazole-7-carboxamide

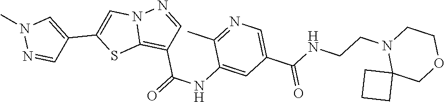 [SMILES: Cc1ncc(C(=O)NCCN2CCOCC23CCC3)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (130 mg, 0.34 mmol), HATU (190 mg, 0.51 mmol) and N,N-diisopropylethylamine (0.22 mL, 1.4 mmol) in DMF (3 mL) was added 2-(8-oxa-5-azaspiro[3.5]nonan-5-yl)ethanamine (120 mg, 0.41 mmol). The resulting mixture was stirred at room temperature for 2 hours and then concentrated under vacuum to give the crude product, which was purified by preparative high-performance liquid chromatography over column: Welch Xtimate C18 150*30 mm*5 um to give the title compound N-(5-((2-(8-oxa-5-azaspiro[3.5]nonan-5-yl)ethyl)carbamoyl)-2-methylpyridin-3-yl)-2-(1-methyl-1H-pyrazol-4-yl)pyrazolo[5,1-b]thiazole-7-carboxamide (29 mg, 16%) as a white solid. LCMS (ESI): mass calcd. for C₂₆H₃₀N₈O₃S, 534.2; m/z found, 535.3 [M+H]⁺. ¹H NMR (400 MHz, METHANOL-d₄) δ 8.78 (d, J=2.0 Hz, 1H), 8.43 (s, 1H), 8.32 (d, J=1.8 Hz, 1H), 8.26 (s, 1H), 8.06 (s, 1H), 7.85 (s, 1H), 3.97 (s, 3H), 3.76-3.67 (m, 2H), 3.60 (s, 2H), 3.50 (t, J=6.8 Hz, 2H), 2.83 (br t, J=6.4 Hz, 2H), 2.63 (s, 3H), 2.23-2.19 (m, 2H), 2.05 (br d, J=5.8 Hz, 2H), 1.76 (br s, 2H), 0.92 (br t, J=6.7 Hz, 2H).

### Example 118: N-(5-((2-(5-azaspiro[2.4]heptan-5-yl)ethyl)carbamoyl)-2-methylpyridin-3-yl)-2-(1-methyl-1H-pyrazol-4-yl)pyrazolo[5,1-b]thiazole-7-carboxamide

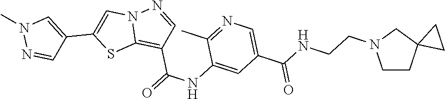 [SMILES: Cc1ncc(C(=O)NCCN2CCC3(CC3)C2)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (100 mg, 0.26 mmol), HATU (149 mg, 0.4 mmol) and N,N-diisopropylethylamine (0.17 mL, 1.0 mmol) in DMF (2 mL) was added 2-(5-azaspiro[2.4]heptan-5-yl)ethanamine (72 mg, 0.34 mmol). The resulting mixture was stirred at room temperature for 2 hours and then concentrated under vacuum to give the crude product, which was purified by preparative high-performance liquid chromatography over column: Welch Xtimate C18 150*30 mm*5 um to give the title compound N-(5-((2-(5-azaspiro[2.4]heptan-5-yl)ethyl)carbamoyl)-2-methylpyridin-3-yl)-2-(1-methyl-1H-pyrazol-4-yl)pyrazolo[5,1-b]thiazole-7-carboxamide (19 mg, 14%) as a white solid. LCMS (ESI): mass calcd. for C₂₅H₂₈N₈O₂S, 504.2; m/z found, 505.4 [M+H]⁺. ¹H NMR (400 MHz, METHANOL-d₄) δ 8.82 (d, J=2.0 Hz, 1H), 8.43 (s, 1H), 8.34 (d, J=2.0 Hz, 1H), 8.26 (s, 1H), 8.06 (s, 1H), 7.85 (s, 1H), 3.97 (s, 3H), 3.60 (t, J=6.7 Hz, 2H), 2.91 (br t, J=6.9 Hz, 2H), 2.81 (br t, J=6.6 Hz, 2H), 2.69 (s, 2H), 2.63 (s, 3H), 1.90 (t, J=7.0 Hz, 2H), 0.67-0.63 (m, 2H), 0.62-0.58 (m, 2H).

### Example 119: N-(5-((2-(4,4-difluoropiperidin-1-yl)ethyl)carbamoyl)-2-methylpyridin-3-yl)-2-(1-methyl-1H-pyrazol-4-yl)pyrazolo[5,1-b]thiazole-7-carboxamide

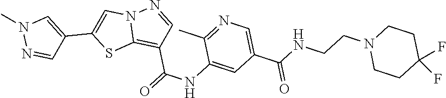 [SMILES: Cc1ncc(C(=O)NCCN2CCC(F)(F)CC2)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (130 mg, 0.14 mmol), HATU (77 mg, 0.20 mmol) and N,N-diisopropylethylamine (0.09 mL, 0.54 mmol) in DMF (3 mL) was added 2-(4,4-difluoropiperidin-1-yl)ethanamine (29 mg, 0.18 mmol). The resulting mixture was stirred at room temperature for 2 hours and then concentrated under vacuum to give the crude product, which was purified by preparative high-performance liquid chromatography over column: Boston Prime C18 150*30 mm*5 um to give the title compound N-(5-((2-(4,4-difluoropiperidin-1-yl)ethyl)carbamoyl)-2-methylpyridin-3-yl)-2-(1-methyl-1H-pyrazol-4-yl)pyrazolo[5,1-b]thiazole-7-carboxamide (29 mg, 38%) as a gray solid. LCMS (ESI): mass calcd. for C₂₄H₂₆F₂N₈O₂S, 528.2; m/z found, 529.2 [M+H]⁺. ¹H NMR (400 MHz, METHANOL-d₄) δ 8.77 (d, J=1.8 Hz, 1H), 8.41 (s, 1H), 8.31 (d, J=1.8 Hz, 1H), 8.24 (s, 1H), 8.04 (s, 1H), 7.83 (s, 1H), 3.95 (s, 3H), 3.56 (t, J=6.6 Hz, 2H), 3.02 (br s, 2H), 2.64-2.56 (m, 5H), 2.09-1.90 (m, 6H).

### Example 120: N-(5-((2-(3,3-difluoropyrrolidin-1-yl)ethyl)carbamoyl)-2-methylpyridin-3-yl)-2-(1-methyl-1H-pyrazol-4-yl)pyrazolo[5,1-b]thiazole-7-carboxamide

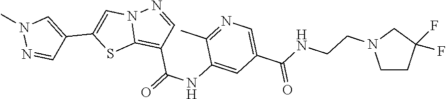 [SMILES: Cc1ncc(C(=O)NCCN2CCC(F)(F)C2)cc1NC(=O)c1cnn2cc(-c3cnn(C)c3)sc12]

### Step a: 2-(3,3-difluoropyrrolidin-1-yl)acetonitrile

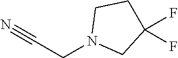

To a solution of 3,3-difluoropyrrolidine hydrochloride (600 mg, 4.2 mmol) and potassium carbonate (1.7 g, 12 mmol) in acetonitrile (12 mL) was added 2-bromoacetonitrile (752 mg, 6.3 mmol) at room temperature. The resulting mixture was stirred at 50° C. for 16 hours before cooling to room temperature. The resulting mixture was quenched with water (15 mL) and extracted with ethyl acetate (30 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=3:1) to give the title compound 2-(3,3-difluoropyrrolidin-1-yl)acetonitrile (400 mg, 66%) as a colorless oil. ¹H NMR (400 MHz, CDCl₃) δ 3.65 (s, 2H), 3.04 (t, J=12.7 Hz, 2H), 2.90 (t, J=7.1 Hz, 2H), 2.34 (tt, J=7.1, 14.5 Hz, 2H).

### Step b: 2-(3,3-difluoropyrrolidin-1-yl)ethanamine

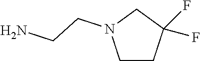

To a solution of 2-(3,3-difluoropyrrolidin-1-yl)acetonitrile (500 mg, 3.4 mmol) in THE (10 mL) was added lithium aluminium hydride (195 mg, 5.1 mmol) by portions at 0° C. (ice/water). The resultant mixture was stirred at room temperature for 3 hours before quenched with water (0.2 mL) at 0° C. The reaction mixture was filtered. And the filtrate was concentrated to dryness under reduced pressure to afford the crude product 2-(3,3-difluoropyrrolidin-1-yl)ethanamine (350 mg, 68%) as a colorless oil, which was used to the next step without further purification. ¹H NMR (400 MHz, CDCl₃) δ 2.88 (t, J=13.3 Hz, 2H), 2.80-2.70 (m, 4H), 2.56-2.50 (m, 2H), 2.25 (tt, J=7.1, 14.6 Hz, 2H).

### Step c: N-(5-((2-(3,3-difluoropyrrolidin-1-yl)ethyl)carbamoyl)-2-methylpyridin-3-yl)-2-(1-methyl-1H-pyrazol-4-yl)pyrazolo[5,1-b]thiazole-7-carboxamide

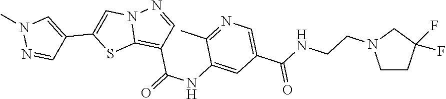 [SMILES: Cc1ncc(C(=O)NCCN2CCC(F)(F)C2)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (130 mg, 0.14 mmol), HATU (77 mg, 0.20 mmol) and N,N-diisopropylethylamine (0.10 mL, 0.6 mmol) in N,N-dimethylformamide (3 mL) was added 2-(3,3-difluoropyrrolidin-1-yl)ethanamine (39 mg, 0.18 mmol). The mixture stirred at room temperature for 2 hours and then concentrated under vacuum to give the crude product, which was purified by preparative high-performance liquid chromatography over column: Boston Prime C18 150*30 mm*5 um to give the title compound N-(5-((2-(3,3-difluoropyrrolidin-1-yl)ethyl)carbamoyl)-2-methylpyridin-3-yl)-2-(1-methyl-1H-pyrazol-4-yl)pyrazolo[5,1-b]thiazole-7-carboxamide (25 mg, 35%) as a gray solid. LCMS (ESI): mass calcd. for C₂₃H₂₄F₂N₈O₂S, 514.2; m/z found, 515.3 [M+H]⁺. ¹H NMR (400 MHz, METHANOL-d₄) δ 8.77 (d, J=1.8 Hz, 1H), 8.41 (s, 1H), 8.29 (d, J=1.8 Hz, 1H), 8.24 (s, 1H), 8.03 (s, 1H), 7.83 (s, 1H), 3.94 (s, 3H), 3.54 (t, J=6.5 Hz, 2H), 3.00 (t, J=13.2 Hz, 2H), 2.84 (t, J=6.9 Hz, 2H), 2.74 (t, J=6.5 Hz, 2H), 2.61 (s, 3H), 2.28 (td, J=7.4, 14.7 Hz, 2H).

### Example 121: N-(5-((2-(1,4-oxazepan-4-yl)ethyl)carbamoyl)-2-methylpyridin-3-yl)-2-(1-methyl-1H-pyrazol-4-yl)pyrazolo[5,1-b]thiazole-7-carboxamide

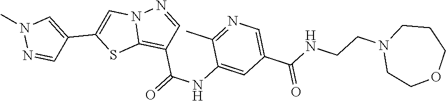 [SMILES: Cc1ncc(C(=O)NCCN2CCCOCC2)cc1NC(=O)c1cnn2cc(-c3cnn(C)c3)sc12]

### Step a: 2-(1,4-oxazepan-4-yl)acetonitrile

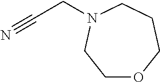

To a solution of 1,4-oxazepane (0.8 g, 7.9 mmol) and potassium carbonate (2.7 g, 20 mmol) in acetonitrile (15 mL) was added 2-bromoacetonitrile (0.54 mL, 8.7 mmol) at room temperature. The resulting mixture was stirred at 50° C. for 12 h before cooling to room temperature. The reaction mixture was filtered. The residue was washed with ethyl acetate (50 mL×3) and concentrated under reduced pressure to give crude product, which was purified by column chromatography over silica gel (eluent: petroleum ether:ethyl acetate=2:1) to give the title compound 2-(1,4-oxazepan-4-yl)acetonitrile (880 mg, 79%) as a yellow liquid. LCMS (ESI): mass calcd. for C₇H₁₂N₂O 140.1; m/z found, 141.2 [M+H]⁺. ¹H NMR (400 MHz, CHLOROFORM-d) δ 3.80 (t, J=6.1 Hz, 2H), 3.76-3.69 (m, 2H), 3.58 (s, 2H), 2.84-2.73 (m, 4H), 1.94 (quin, J=5.9 Hz, 2H).

### Step b: 2-(1,4-oxazepan-4-yl)ethanamine

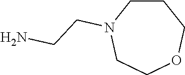

To a solution of 2-(1,4-oxazepan-4-yl)acetonitrile (880 mg, 6.2 mmol) in THE (30 mL) was added lithium aluminium hydride (480 mg, 13 mmol) by portions at 0° C. (ice/water), and the resulting mixture was stirred at 25° C. for 1 h. After cooled to 0° C., the reaction mixture was quenched with water (0.25 mL) and filtered. The filtrate was concentrated to dryness under reduced pressure to afford the crude product 2-(1,4-oxazepan-4-yl)ethanamine (700 mg, 77%) as colorless liquid. LCMS (ESI): mass calcd. for C₇H₁₆N20, 144.2; m/z found, 145.3[M+H]⁺. ¹H NMR (400 MHz, CHLOROFORM-d) δ 3.78 (t, J=6.1 Hz, 2H), 3.72-3.67 (m, 2H), 2.77-2.64 (m, 6H), 2.58-2.50 (m, 2H), 1.87 (quin, J=5.9 Hz, 2H).

### Step c: N-(5-((2-(1,4-oxazepan-4-yl)ethyl)carbamoyl)-2-methylpyridin-3-yl)-2-(1-methyl-1H-pyrazol-4-yl)pyrazolo[5,1-b]thiazole-7-carboxamide

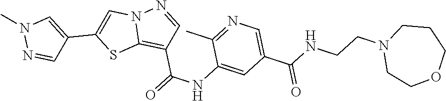 [SMILES: Cc1ncc(C(=O)NCCN2CCCOCC2)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (100 mg, 0.26 mmol), HATU (150 mg, 0.39 mmol) and DIEA (0.17 mL, 1.05 mmol) in DMF (5 mL) was added 2-(1,4-oxazepan-4-yl)ethanamine (49 mg, 0.34 mmol). The mixture stirred at room temperature for 2 h and then concentrated under vacuum to give the crude product, which was purified by preparative high-performance liquid chromatography over column: Welch Xtimate C18 150*30 mm*5 um to give the title compound N-(5-((2-(1,4-oxazepan-4-yl)ethyl)carbamoyl)-2-methylpyridin-3-yl)-2-(1-methyl-1H-pyrazol-4-yl)pyrazolo[5,1-b]thiazole-7-carboxamide (67 mg, 28%) as a white solid. LCMS (ESI): mass calcd. for C₂₄H₂₈N₈O₃S, 508.6 m/z found, 509.2 [M+H]⁺. ¹H NMR (400 MHz, METHANOL-d₄) δ 8.77 (d, J=2.0 Hz, 1H), 8.40 (s, 1H), 8.31 (d, J=2.0 Hz, 1H), 8.24 (s, 1H), 8.03 (s, 1H), 7.82 (s, 1H), 3.94 (s, 3H), 3.83-3.75 (m, 4H), 3.58 (t, J=6.5 Hz, 2H), 3.06-2.84 (m, 8H), 2.61 (s, 3H), 1.96 (quin, J=5.8 Hz, 2H).

### Example 122: N-(5-((2-(3-oxa-8-azabicyclo[3.2.1]octan-8-yl)ethyl)carbamoyl)-2-methylpyridin-3-yl)-2-(1-methyl-1H-pyrazol-4-yl)pyrazolo[5,1-b]thiazole-7-carboxamide

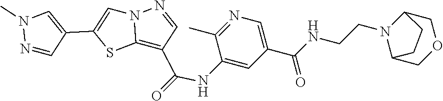 [SMILES: Cc1ncc(C(=O)NCCN2C3CCC2COC3)cc1NC(=O)c1cnn2cc(-c3cnn(C)c3)sc12]

### Step a: 2-(3-oxa-8-azabicyclo[3.2.1]octan-8-yl)acetonitrile

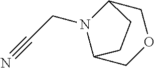 [SMILES: N#CCN1C2CCC1COC2]

To a solution of 3-oxa-8-azabicyclo[3.2.1]octane; hydrochloride (0.4 g, 2.7 mmol) and potassium carbonate (0.92 g, 6.7 mmol) in acetonitrile (6 mL) was added 2-bromoacetonitrile (0.18 mL, 2.9 mmol) at room temperature. The resulting mixture was stirred at 50° C. for 12 h before cooling to room temperature. The reaction mixture was filtered. The residue was washed with ethyl acetate (50 mL×3) and concentrated under reduced pressure to give crude product, which was purified by column chromatography over silica gel (eluent: petroleum ether:ethyl acetate=2:1) to give the title compound 2-(3-oxa-8-azabicyclo[3.2.1]octan-8-yl)acetonitrile (402 mg, 99%) as a yellow liquid. LCMS (ESI): mass calcd. for C₈H₁₂N₂O: 152.2; m/z found, 153.2 [M+H]⁺. ¹H NMR (400 MHz, CHLOROFORM-d) δ 3.73 (d, J=10.6 Hz, 2H), 3.55 (br d, J=10.6 Hz, 2H), 3.27 (s, 2H), 3.19 (br s, 2H), 2.03-1.86 (m, 4H).

### Step b: 2-(3-oxa-8-azabicyclo[3.2.1]octan-8-yl)ethanamine

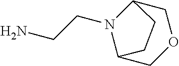 [SMILES: NCCN1C2CCC1COC2]

To a solution of 2-(1,4-oxazepan-4-yl)acetonitrile (370 mg, 2.4 mmol) in THE (15 mL) was added lithium aluminium hydride (180 mg, 4.9 mmol) by portions at 0° C. (ice/water), and the resulting mixture was stirred at 25° C. for 1 h. After cooled to 0° C., the reaction mixture was quenched with water (0.25 mL) and filtered. The filtrate was concentrated to dryness under reduced pressure to afford the crude product 2-(3-oxa-8-azabicyclo[3.2.1]octan-8-yl)ethanamine (350 mg, 91%) as colorless liquid. LCMS (ESI): mass calcd. for C₈H₁₆N₂O, 156.2; m/z found, 157.2 [M+H]⁺. ¹H NMR (400 MHz, CHLOROFORM-d) δ 3.68 (d, J=10.4 Hz, 2H), 3.51 (br d, J=10.1 Hz, 2H), 3.00 (br s, 2H), 2.76-2.68 (m, 2H), 2.32 (t, J=5.8 Hz, 2H), 1.86 (s, 4H).

### Step c: N-(5-((2-(3-oxa-8-azabicyclo[3.2.1]octan-8-yl)ethyl)carbamoyl)-2-methylpyridin-3-yl)-2-(1-methyl-1H-pyrazol-4-yl)pyrazolo[5,1-b]thiazole-7-carboxamide

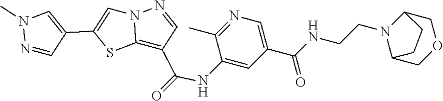 [SMILES: Cc1ncc(C(=O)NCCN2C3CCC2COC3)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (120 mg, 0.31 mmol), HATU (180 mg, 0.47 mmol) and DIEA (0.21 mL, 1.3 mmol) in DMF (5 mL) was added 2-(3-oxa-8-azabicyclo[3.2.1]octan-8-yl)ethanamine (64 mg, 0.41 mmol). The mixture stirred at room temperature for 2 h and then concentrated under vacuum to give the crude product, which was purified by preparative high-performance liquid chromatography over column: Welch Xtimate C18 150*30 mm*5 um and by Supercritical Fluid Chromatography over column: DAICEL CHIRALCEL OD-H (250 mm*30 mm, 5 um) to give the title compound N-(5-((2-(3-oxa-8-azabicyclo[3.2.1]octan-8-yl)ethyl)carbamoyl)-2-methylpyridin-3-yl)-2-(1-methyl-1H-pyrazol-4-yl)pyrazolo[5,1-b]thiazole-7-carboxamide (27 mg, 16%) as a white solid. LCMS (ESI): mass calcd. for C₂₅H₂₈N₈O₃S, 520.6 m/z found, 521.3 [M+H]⁺. ¹H NMR (400 MHz, METHANOL-d₄) δ 8.77 (d, J=2.0 Hz, 1H), 8.40 (s, 1H), 8.31 (d, J=1.8 Hz, 1H), 8.24 (s, 1H), 8.03 (s, 1H), 7.82 (s, 1H), 3.94 (s, 3H), 3.72 (d, J=10.6 Hz, 2H), 3.56-3.50 (m, 4H), 3.21 (br s, 2H), 2.65-2.55 (m, 5H), 2.04-1.95 (m, 2H), 1.92-1.84 (m, 2H).

### Example 123: N-(5-((2-(3,3-difluoropiperidin-1-yl)ethyl)carbamoyl)-2-methylpyridin-3-yl)-2-(1-methyl-1H-pyrazol-4-yl)pyrazolo[5,1-b]thiazole-7-carboxamide

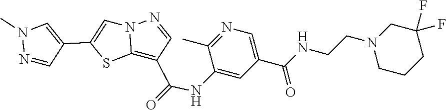 [SMILES: Cc1ncc(C(=O)NCCN2CCCC(F)(F)C2)cc1NC(=O)c1cnn2cc(-c3cnn(C)c3)sc12]

### Step a: 2-(3,3-difluoropiperidin-1-yl)acetonitrile

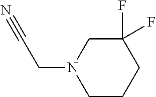 [SMILES: N#CCN1CCCC(F)(F)C1]

To a solution of 3,3-difluoropiperidin-1-ium chloride (0.5 g, 3.2 mmol) and potassium carbonate (1.1 g, 7.9 mmol) in acetonitrile (7 mL) was added 2-bromoacetonitrile (0.22 mL, 3.5 mmol) at room temperature. The resulting mixture was stirred at 50° C. for 12 h before cooling to room temperature. The reaction mixture was filtered. The residue was washed with ethyl acetate (50 mL×3) and concentrated under reduced pressure to give crude product, which was purified by column chromatography over silica gel (eluent: petroleum ether:ethyl acetate=2:1) to give the title compound 2-(3,3-difluoropiperidin-1-yl)acetonitrile (506 mg, 99%) as a yellow liquid. LCMS (ESI): mass calcd. for C₇H₁₀F₂N₂, 160.2; m/z found, 161.1 [M+H]⁺. ¹H NMR (400 MHz, CHLOROFORM-d) δ 3.63 (s, 2H), 2.80 (t, J=11.0 Hz, 2H), 2.61 (br t, J=5.2 Hz, 2H), 2.01-1.80 (m, 4H).

### Step b: 2-(3,3-difluoropiperidin-1-yl)ethanamine

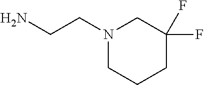 [SMILES: NCCN1CCCC(F)(F)C1]

To a solution of 2-(3,3-difluoropiperidin-1-yl)acetonitrile (480 mg, 3.0 mmol) in THE (15 mL) was added lithium aluminium hydride (230 mg, 6.0 mmol) by portions at 0° C. (ice/water), and the resulting mixture was stirred at 25° C. for 1 h. After cooled to 0° C., the reaction mixture was quenched with water (0.25 mL) and filtered. The filtrate was concentrated to dryness under reduced pressure to afford the crude product 2-(3,3-difluoropiperidin-1-yl)ethanamine (430 mg, 88%) as colorless liquid. LCMS (ESI): mass calcd. for C₇H₁₄F₂N₂, 164.2; m/z found, 165.2 [M+H]⁺. ¹H NMR (400 MHz, CHLOROFORM-d) δ 2.81 (br t, J=6.0 Hz, 2H), 2.65 (br t, J=11.4 Hz, 2H), 2.56-2.41 (m, 4H), 1.95-1.82 (m, 4H), 1.81-1.71 (m, 2H).

### Step c: N-(5-((2-(3,3-difluoropiperidin-1-yl)ethyl)carbamoyl)-2-methylpyridin-3-yl)-2-(1-methyl-1H-pyrazol-4-yl)pyrazolo[5,1-b]thiazole-7-carboxamide

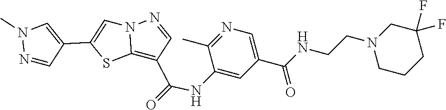 [SMILES: Cc1ncc(C(=O)NCCN2CCCC(F)(F)C2)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (120 mg, 0.31 mmol), HATU (180 mg, 0.47 mmol) and DIEA (0.21 mL, 1.3 mmol) in DMF (3 mL) was added 2-(3,3-difluoropiperidin-1-yl)ethanamine (67 mg, 0.41 mmol). The mixture stirred at room temperature for 2 h and then concentrated under vacuum to give the crude product, which was purified by preparative high-performance liquid chromatography over column: Phenomenex Gemini-NX 80*40 mm*3 um and by Supercritical Fluid Chromatography over column: DAICEL CHIRALCEL OD-H (250 mm*30 mm, 5 um) to give the title compound N-(5-((2-(3,3-difluoropiperidin-1-yl)ethyl)carbamoyl)-2-methylpyridin-3-yl)-2-(1-methyl-1H-pyrazol-4-yl)pyrazolo[5,1-b]thiazole-7-carboxamide (27 mg, 16%) as a white solid. LCMS (ESI): mass calcd. for C₂₄H₂₆F₂N₈O₂S, 528.6 m/z found, 529.2 [M+H]⁺. ¹H NMR (400 MHz, METHANOL-d₄) δ 8.77 (d, J=2.0 Hz, 1H), 8.41 (s, 1H), 8.29 (d, J=2.0 Hz, 1H), 8.25 (s, 1H), 8.04 (s, 1H), 7.84 (s, 1H), 3.95 (s, 3H), 3.56 (t, J=6.6 Hz, 2H), 2.76 (t, J=11.2 Hz, 2H), 2.70 (t, J=6.6 Hz, 2H), 2.61 (s, 3H), 2.60-2.55 (m, 2H), 1.90 (tt, J=6.5, 13.2 Hz, 2H), 1.82-1.74 (m, 2H).

### Example 124: N-(5-((2-(2-azaspiro[3.3]heptan-2-yl)ethyl)carbamoyl)-2-methylpyridin-3-yl)-2-(1-methyl-1H-pyrazol-4-yl)pyrazolo[5,1-b]thiazole-7-carboxamide

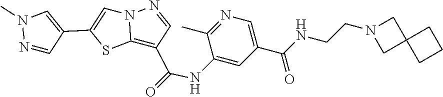 [SMILES: Cc1ncc(C(=O)NCCN2CC3(CCC3)C2)cc1NC(=O)c1cnn2cc(-c3cnn(C)c3)sc12]

### Step a: 2-(2-azaspiro[3.3]heptan-2-yl)acetonitrile

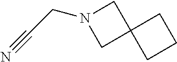

To a solution of 2-azaspiro[3.3]heptane; hydrochloride (0.5 g, 3.7 mmol) and potassium carbonate (1.3 g, 9.4 mmol) in acetonitrile (7 mL) was added 2-bromoacetonitrile (0.26 mL, 4.1 mmol) at room temperature. The resulting mixture was stirred at 50° C. for 12 h before cooling to room temperature. The reaction mixture was filtered. The residue was washed with ethyl acetate (50 mL×3) and concentrated under reduced pressure to give crude product, which was purified by column chromatography over silica gel (eluent: petroleum ether:ethyl acetate=2:1) to give the title compound 2-(2-azaspiro[3.3]heptan-2-yl)acetonitrile (500 mg, 98%) as a yellow liquid. LCMS (ESI): mass calcd. for C₈H₁₂N₂, 136.2; m/z found, 137.2 [M+H]⁺. ¹H NMR (400 MHz, CHLOROFORM-d) δ 3.69-3.58 (m, 2H), 3.56-3.15 (m, 3H), 2.99-2.71 (m, 1H), 2.22-2.09 (m, 2H), 2.08-1.70 (m, 4H).

### Step b: 2-(2-azaspiro[3.3]heptan-2-yl)ethanamine

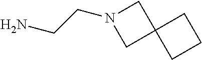

To a solution of 2-(2-azaspiro[3.3]heptan-2-yl)acetonitrile (470 mg, 3.5 mmol) in THE (15 mL) was added lithium aluminium hydride (260 mg, 7.0 mmol) by portions at 0° C. (ice/water), and the resulting mixture was stirred at 25° C. for 1 h. After cooled to 0° C., the reaction mixture was quenched with water (0.25 mL) and filtered. The filtrate was concentrated to dryness under reduced pressure to afford the crude product 2-(2-azaspiro[3.3]heptan-2-yl)ethanamine (250 mg, 52%) as colorless liquid. LCMS (ESI): mass calcd. for C₈H_{1M}N₂, 140.2; m/z found, 141.2 [M+H]⁺. ¹H NMR (400 MHz, CHLOROFORM-d) δ 3.16 (s, 4H), 2.64 (t, J=6.3 Hz, 2H), 2.45-2.41 (m, 2H), 2.12-2.05 (m, 5H), 1.84-1.76 (m, 3H).

### Step c: N-(5-((2-(2-azaspiro[3.3]heptan-2-yl)ethyl)carbamoyl)-2-methylpyridin-3-yl)-2-(1-methyl-1H-pyrazol-4-yl)pyrazolo[5,1-b]thiazole-7-carboxamide

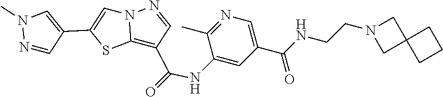 [SMILES: Cc1ncc(C(=O)NCCN2CC3(CCC3)C2)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (130 mg, 0.14 mmol), HATU (77 mg, 0.20 mmol) and DIEA (90 μL, 0.54 mmol) in DMF (5 mL) was added 2-(2-azaspiro[3.3]heptan-2-yl)ethanamine (25 mg, 0.18 mmol). The mixture stirred at room temperature for 2 h and then concentrated under vacuum to give the crude product, which was purified by preparative high-performance liquid chromatography over column: Welch Xtimate C18 150*30 mm*5 um to give the title compound N-(5-((2-(2-azaspiro[3.3]heptan-2-yl)ethyl)carbamoyl)-2-methylpyridin-3-yl)-2-(1-methyl-1H-pyrazol-4-yl)pyrazolo[5,1-b]thiazole-7-carboxamide (25 mg, 35%) as a white solid. LCMS (ESI): mass calcd. for C₂₅H₂₈N₈O₂S, 504.6 m/z found, 505.3 [M+H]⁺. ¹H NMR (400 MHz, METHANOL-d₄) δ 8.78 (d, J=2.0 Hz, 1H), 8.41 (s, 1H), 8.31 (d, J=2.0 Hz, 1H), 8.25 (s, 1H), 8.04 (s, 1H), 7.83 (s, 1H), 3.95 (s, 3H), 3.40 (t, J=6.5 Hz, 2H), 3.33 (br s, 4H), 2.69 (t, J=6.5 Hz, 2H), 2.61 (s, 3H), 2.14 (t, J=7.6 Hz, 4H), 1.87-1.81 (m, 2H).

### Example 125: N-(5-((2-(3,3-difluoroazetidin-1-yl)ethyl)carbamoyl)-2-methylpyridin-3-yl)-2-(1-methyl-1H-pyrazol-4-yl)pyrazolo[5,1-b]thiazole-7-carboxamide

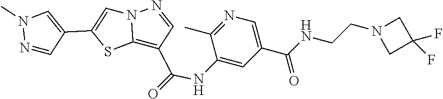 [SMILES: Cc1ncc(C(=O)NCCN2CC(F)(F)C2)cc1NC(=O)c1cnn2cc(-c3cnn(C)c3)sc12]

To a solution of 6-methyl-5-(2-(1-methyl-1H-pyrazol-4-yl)pyrazolo[5,1-b]thiazole-7-carboxamido)nicotinic acid (500 mg, 1.1 mmol), HATU (628 mg, 1.7 mmol) and N,N-diisopropylethylamine (0.73 mL, 4.4 mmol) in DMF (5 mL) was added 2-(3,3-difluoroazetidin-1-yl)ethanamine (180 mg, 1.3 mmol). The resulting mixture was stirred at room temperature for 2 h and then concentrated under vacuum to give the crude product, which was purified by column chromatography over silica gel (eluent: Petroleum ether/ethyl acetate=0:100 to 100:0 then ethyl acetate/methanol=70:30) to give the crude product. The crude product was purified by preparative high-performance liquid chromatography over column: Phenomenex Gemini-NX 80*40 mm*3 um to give the crude product and the crude product was purified by Supercritical Fluid Chromatography over column: DAICEL CHIRALPAK AS 250 mm*30 mm, 5 um to give title compound N-(5-((2-(3,3-difluoroazetidin-1-yl)ethyl)carbamoyl)-2-methylpyridin-3-yl)-2-(1-methyl-1H-pyrazol-4-yl)pyrazolo[5,1-b]thiazole-7-carboxamide (31.8 mg, 5%) as a white solid. LCMS (ESI): mass calcd. for C₂₂H₂₂F₂N₈O₂S, 500.5; m/z found, 501.3 [M+H]⁺. ¹H NMR (400 MHz, METHANOL-d₄) δ 8.78 (d, J=2.0 Hz, 1H), 8.40 (s, 1H), 8.30 (d, J=1.9 Hz, 1H), 8.23 (s, 1H), 8.03 (s, 1H), 7.82 (s, 1H), 3.94 (s, 3H), 3.70 (t, J=12.1 Hz, 4H), 3.45 (t, J=6.3 Hz, 2H), 2.83 (t, J=6.2 Hz, 2H), 2.60 (s, 3H).

### Example 126: N-(5-((2-(3,3-dimethylazetidin-1-yl)ethyl)carbamoyl)-2-methylpyridin-3-yl)-2-(1-methyl-1H-pyrazol-4-yl)pyrazolo[5,1-b]thiazole-7-carboxamide

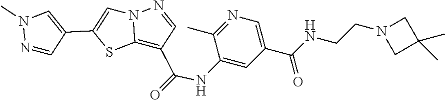

To a solution of 6-methyl-5-(2-(1-methyl-1H-pyrazol-4-yl)pyrazolo[5,1-b]thiazole-7-carboxamido)nicotinic acid (350 mg, 0.59 mmol), HATU (336 mg, 0.88 mmol) and N,N-diisopropylethylamine (0.39 mL, 2.4 mmol) in DMF (5 mL) was added 2-(3,3-dimethylazetidin-1-yl)ethanamine (90 mg, 0.71 mmol). The resulting mixture was stirred at room temperature for 2 h and then concentrated under vacuum to give the crude product, which was purified by column chromatography over silica gel (eluent: Petroleum ether/ethyl acetate=0:100 to 100:0 then ethyl acetate/methanol=70:30) to give the crude product. The crude product was purified by preparative high-performance liquid chromatography over column: Phenomenex Gemini-NX 80*40 mm*3 um to give the crude product and the crude product was purified by Supercritical Fluid Chromatography over column: DAICEL CHIRALPAK AS 250 mm*30 mm, 10 um to give title compound N-(5-((2-(3,3-dimethylazetidin-1-yl)ethyl)carbamoyl)-2-methylpyridin-3-yl)-2-(1-methyl-1H-pyrazol-4-yl)pyrazolo[5,1-b]thiazole-7-carboxamide (38.2 mg, 13%) as a white solid. LCMS (ESI): mass calcd. for C₂₄H₂₈N₈O₂S, 492.6; m/z found, 493.2 [M+H]⁺. ¹H NMR (400 MHz, METHANOL-d₄) δ 8.78 (d, J=2.0 Hz, 1H), 8.41 (s, 1H), 8.31 (d, J=2.0 Hz, 1H), 8.25 (s, 1H), 8.04 (s, 1H), 7.83 (s, 1H), 3.95 (s, 3H), 3.42 (t, J=6.6 Hz, 2H), 3.16 (s, 4H), 2.77 (t, J=6.4 Hz, 2H), 2.61 (s, 3H), 1.25 (s, 6H).

### Example 127: N-(5-((2-(isopropyl(2-methoxyethyl)amino)ethyl)carbamoyl)-2-methylpyridin-3-yl)-2-(1-methyl-1H-pyrazol-4-yl)pyrazolo[5,1-b]thiazole-7-carboxamide

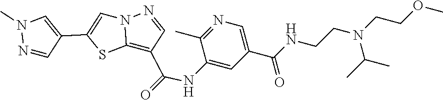

### Step a: 2-(isopropyl(2-methoxyethyl)amino)acetonitrile

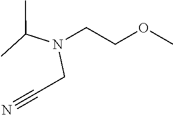

A mixture of N-(2-methoxyethyl)propan-2-amine (450 mg, 3.8 mmol) and K₂CO₃ (1.6 g, 11.5 mmol) in ACN (10 mL), then 2-bromoacetonitrile (0.36 mL, 5.8 mmol) was added. The reaction mixture was stirred at 50° C. for 16 hours. The resulting mixture was quenched with water (20 mL) and extracted with ethyl acetate (50 mL×3). The combined organic phases were dried over anhydrous Na₂SO₄ and filtered. The filtrate was concentrated under reduced pressure to give crude product, which was purified by column chromatography over silica gel (eluent: petroleum ether:ethyl acetate from 100:0 to 65:35) to give the title compound 2-(isopropyl(2-methoxyethyl)amino)acetonitrile (550 mg, 92%) as a colorless liquid. ¹H NMR (400 MHz, CHLOROFORM-d) δ 3.68 (s, 2H), 3.48 (t, J=4.9 Hz, 2H), 3.34 (d, J=1.1 Hz, 3H), 2.93 (spt, J=6.3 Hz, 1H), 2.80 (t, J=5.1 Hz, 2H), 1.14-1.05 (m, 6H).

### Step b: N¹-isopropyl-N¹-(2-methoxyethyl)ethane-1,2-diamine

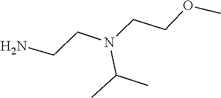

To a solution of 2-(isopropyl(2-methoxyethyl)amino)acetonitrile (550 mg, 3.5 mmol) in THE (10 mL) was added lithium aluminium hydride (200 mg, 5.3 mmol) by portions at 0° C. (ice/water). The resultant mixture was stirred at room temperature for 1.5 h before quenched with water (0.5 mL) at 0° C. The reaction mixture was filtered. And the filtrate was concentrated to dryness under reduced pressure to afford the crude product N¹-isopropyl-N¹-(2-methoxyethyl)ethane-1,2-diamine (470 mg, 83%) as a colorless oil. LCMS (ESI): mass calcd. for C₈H₂₀N₂O, 160.257; m/z found, 161.200 [M+H]⁺. ¹H NMR (400 MHz, CHLOROFORM-d) δ 3.40 (t, J=6.5 Hz, 2H), 3.36 (s, 3H), 2.93 (td, J=6.6, 13.2 Hz, 1H), 2.71-2.66 (m, 2H), 2.61 (t, J=6.7 Hz, 2H), 2.53-2.47 (m, 2H), 1.55-1.43 (m, 2H), 1.00 (d, J=6.5 Hz, 6H).

### Step c: N-(5-((2-(isopropyl(2-methoxyethyl)amino)ethyl)carbamoyl)-2-methylpyridin-3-yl)-2-(1-methyl-1H-pyrazol-4-yl)pyrazolo[5,1-b]thiazole-7-carboxamide

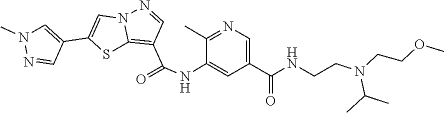

To a solution of 6-methyl-5-(2-(1-methyl-1H-pyrazol-4-yl)pyrazolo[5,1-b]thiazole-7-carboxamido)nicotinic acid (200 mg, 0.44 mmol), HATU (251 mg, 0.66 mmol) and N,N-diisopropylethylamine (0.29 mL, 1.8 mmol) in DMF (3 mL) was added N¹-isopropyl-N¹-(2-methoxyethyl)ethane-1,2-diamine (92 mg, 0.57 mmol). The resulting mixture was stirred at room temperature for 2 h and then concentrated under vacuum to give the crude product, which was purified by preparative high-performance liquid chromatography over column: Phenomenex Gemini-NX C18 80*30 mm*3 um to give the title compound N-(5-((2-(isopropyl(2-methoxyethyl)amino)ethyl)carbamoyl)-2-methylpyridin-3-yl)-2-(1-methyl-1H-pyrazol-4-yl)pyrazolo[5,1-b]thiazole-7-carboxamide (44 mg, 18%) as a white solid. LCMS (ESI): mass calcd. for C₂₅H₃₂N₈O₃S, 524.638; m/z found, 525.40 [M+H]⁺. ¹H NMR (400 MHz, METHANOL-d₄) δ 8.79 (d, J=2.0 Hz, 1H), 8.42 (s, 1H), 8.32 (d, J=2.0 Hz, 1H), 8.26 (s, 1H), 8.06 (s, 1H), 7.85 (s, 1H), 3.97 (s, 3H), 3.53-3.42 (m, 5H), 3.34 (br s, 3H), 3.03 (br s, 2H), 2.72 (br t, J=5.7 Hz, 4H), 2.63 (s, 1H), 1.06 (s, 3H), 1.05 (s, 3H).

### Example 128: N-(5-((2-(bis(2-methoxyethyl)amino)ethyl)carbamoyl)-2-methylpyridin-3-yl)-2-(1-methyl-1H-pyrazol-4-yl)pyrazolo[5,1-b]thiazole-7-carboxamide

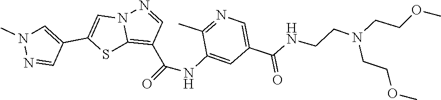

### Step a: 2-(bis(2-methoxyethyl)amino)acetonitrile

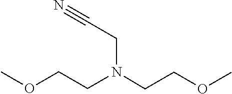

A mixture of bis(2-methoxyethyl)amine (450 mg, 3.4 mmol) and K₂CO₃ (1.4 g, 10.1 mmol) in ACN (10 mL), then 2-bromoacetonitrile (0.32 mL, 5.1 mmol) was added. The reaction mixture was stirred at 50° C. for 16 hours. The resulting mixture was quenched with water (20 mL) and extracted with ethyl acetate (50 mL×3). The combined organic phases were dried over anhydrous Na₂SO₄ and filtered. The filtrate was concentrated under reduced pressure to give crude product, which was purified by column chromatography over silica gel (eluent: petroleum ether:ethyl acetate from 100:0 to 55:45) to give the title compound 2-(bis(2-methoxyethyl)amino)acetonitrile (560 mg, 96%) as a colorless liquid. ¹H NMR (400 MHz, CHLOROFORM-d) δ 3.80 (s, 2H), 3.54 (t, J=5.3 Hz, 4H), 3.37 (s, 6H), 2.82 (t, J=5.1 Hz, 4H).

### Step b: N¹,N¹-bis(2-methoxyethyl)ethane-1,2-diamine

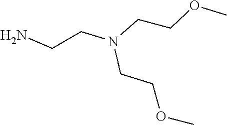

To a solution of 2-(bis(2-methoxyethyl)amino)acetonitrile (560 mg, 3.3 mmol) in THE (10 mL) was added lithium aluminium hydride (185 mg, 4.9 mmol) by portions at 0° C. (ice/water). The resultant mixture was stirred at room temperature for 1.5 h before quenched with water (0.5 mL) at 0° C. The reaction mixture was filtered. And the filtrate was concentrated to dryness under reduced pressure to afford the crude product N¹,N¹-bis(2-methoxyethyl)ethane-1,2-diamine (470 mg, 82%) as a colorless oil. LCMS (ESI): mass calcd. for C₈H₂₀N₂O₂, 176.257; m/z found, 177.200 [M+H]⁺. ¹H NMR (400 MHz, CHLOROFORM-d) δ 3.48 (t, J=6.1 Hz, 4H), 3.38-3.34 (m, 6H), 2.78-2.71 (m, 6H), 2.65-2.58 (m, 2H), 1.39 (br s, 2H).

### Step c: N-(5-((2-(bis(2-methoxyethyl)amino)ethyl)carbamoyl)-2-methylpyridin-3-yl)-2-(1-methyl-1H-pyrazol-4-yl)pyrazolo[5,1-b]thiazole-7-carboxamide

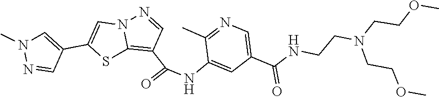

To a solution of 6-methyl-5-(2-(1-methyl-1H-pyrazol-4-yl)pyrazolo[5,1-b]thiazole-7-carboxamido)nicotinic acid (200 mg, 0.44 mmol), HATU (250 mg, 0.66 mmol) and N,N-diisopropylethylamine (0.29 mL, 1.8 mmol) in DMF (3 mL) was added N¹,N¹-bis(2-methoxyethyl)ethane-1,2-diamine (101 mg, 0.57 mmol). The resulting mixture was stirred at room temperature for 2 h and then concentrated under vacuum to give the crude product, which was purified by preparative high-performance liquid chromatography over column: Phenomenex Gemini-NX 80*40 mm*3 um to give the title compound N-(5-((2-(bis(2-methoxyethyl)amino)ethyl)carbamoyl)-2-methylpyridin-3-yl)-2-(1-methyl-1H-pyrazol-4-yl)pyrazolo[5,1-b]thiazole-7-carboxamide (34 mg, 14%) as a white solid. LCMS (ESI): mass calcd. for C₂₅H₃₂N₈O₄S, 540.638; m/z found, 541.50 [M+H]⁺. ¹H NMR (400 MHz, METHANOL-d₄) δ 8.80 (d, J=2.0 Hz, 1H), 8.42 (s, 1H), 8.32 (d, J=2.0 Hz, 1H), 8.26 (s, 1H), 8.06 (s, 1H), 7.85 (s, 1H), 3.97 (s, 3H), 3.54-3.49 (m, 7H), 3.34 (br s, 4H), 2.84-2.78 (m, 7H), 2.63 (s, 3H).

### Example 129: N-(5-((2-(4-acetylpiperazin-1-yl)ethyl)carbamoyl)-2-methylpyridin-3-yl)-2-(1-methyl-1H-pyrazol-4-yl)pyrazolo[5,1-b]thiazole-7-carboxamide

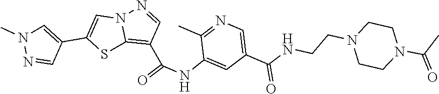 [SMILES: CC(=O)N1CCN(CCNC(=O)c2cnc(C)c(NC(=O)c3cnn4cc(-c5cnn(C)c5)sc34)c2)CC1]

### Step a: tert-butyl (2-(4-acetylpiperazin-1-yl)ethyl)carbamate

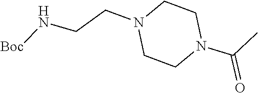 [SMILES: CC(=O)N1CCN(CCNC(=O)OC(C)(C)C)CC1]

To a solution of 1-(piperazin-1-yl)ethanone (2 g, 16 mmol) was added to the tert-butyl (bromomethyl)carbamate (3.5 g, 16 mmol), and K₂CO₃ (4.3 g, 31 mmol) in MeCN (30 mL) was added 2-(2,6-dimethylpiperidin-1-yl)ethanamine (565 mg, 3.6 mmol). The mixture stirred at 80° C. for 16 hours and water (150 mL) was added, and the mixture was extracted with ethyl acetate (100 mL*3). The organic layer was collected, dried over anhydrous Na₂SO₄, filtered and evaporated under vacuum to give the crude product as yellow oil, The residue was purified by preparative thin layer chromatography (eluent: eluent: petroleum ether:ethyl acetate=100/0 to petroleum ether:ethyl acetate=0/100) to give pure product tert-butyl (2-(4-acetylpiperazin-1-yl)ethyl)carbamate (2.1 g, 44%) as a colorless oil. LCMS (ESI): mass calcd. for C₁₃H₂₅N₃O₃, 271.3; m/z found, 272 [M+H]⁺. ¹H NMR (400 MHz, METHANOL-d₄) δ 3.54-3.59 (m, 2H), 3.49-3.54 (m, 2H), 3.18 (t, J=6.62 Hz, 2H), 2.40-2.53 (m, 6H), 2.07 (s, 3H), 1.45-1.45 (m, 1H) 1.39-1.45 (m, 9H).

### Step b: tert-butyl (2-(4-acetylpiperazin-1-yl)ethyl)carbamate

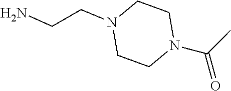

To a solution of 1-(piperazin-1-yl)ethanone (2 g, 16 mmol) was added to the tert-butyl (bromomethyl)carbamate (3.5 g, 16 mmol) and K₂CO₃ (4.3 g, 31 mmol) in MeCN (30 mL) was added 2-(2,6-dimethylpiperidin-1-yl)ethanamine (565 mg, 3.6 mmol). The mixture stirred at 80° C. for 16 hours and water (150 mL) was added, and the mixture was extracted with ethyl acetate (100 mL*3). The organic layer was collected, dried over anhydrous Na₂SO₄, filtered and evaporated under vacuum to give the crude product as yellow oil, The residue was purified by preparative thin layer chromatography (eluent: eluent: petroleum ether:ethyl acetate=100/0 to petroleum ether:ethyl acetate=0/100) to give pure product tert-butyl (2-(4-acetylpiperazin-1-yl)ethyl)carbamate (2.1 g, 44%) as a colorless oil. LCMS (ESI): mass calcd. for C₁₃H₂₅N₃O₃, 271.3; m/z found, 272 [M+H]⁺. ¹H NMR (400 MHz, METHANOL-d₄) δ 3.54-3.59 (m, 2H), 3.49-3.54 (m, 2H), 3.18 (t, J=6.62 Hz, 2H), 2.40-2.53 (m, 6H), 2.07 (s, 3H), 1.45-1.45 (m, 1H) 1.39-1.45 (m, 9H)

### Step c: N-(5-((2-(4-acetylpiperazin-1-yl)ethyl)carbamoyl)-2-methylpyridin-3-yl)-2-bromopyrazolo[5,1-b]thiazole-7-carboxamide

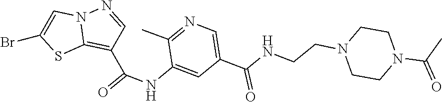 [SMILES: CC(=O)N1CCN(CCNC(=O)c2cnc(C)c(NC(=O)c3cnn4cc(Br)sc34)c2)CC1]

To a solution of 5-(2-bromopyrazolo[5,1-b]thiazole-7-carboxamido)-6-methylnicotinic acid (200 mg, 0.18 mmol) and DIEA (146 μL, 0.89 mmol) in DMF (6 mL) was added 1-(4-(2-aminoethyl)piperazin-1-yl)ethanone (61 mg, 0.35 mmol) and HATU (161 mg, 0.43 mmol). The mixture was stirred at 35° C. for 16 hours and then concentrated under vacuum to give the crude product, which was purified by preparative high-performance liquid chromatography over column: Xtimate C18 100*30 mm*3 um and by Supercritical Fluid Chromatography over column: DAICEL CHIRALPAK AS (250 mm*30 mm, 10 um) to give the title compound N-(5-((2-(4-acetylpiperazin-1-yl)ethyl)carbamoyl)-2-methylpyridin-3-yl)-2-bromopyrazolo[5,1-b]thiazole-7-carboxamide (70 mg, 34%) as a white solid. LCMS (ESI): mass calcd. for C₂₁H₂₄BrN₇O₃S, 533.4; m/z found, 534.1 [M+H]⁺

### Step d: N-(2-(2,6-dimethylpiperidin-1-yl)ethyl)-6-methyl-5-((1-methyl-6-((1-methyl-11H-pyrazol-4-yl)amino)-1H-pyrazolo[3,4-d]pyrimidin-3-yl)amino)nicotinamide

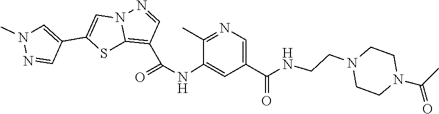 [SMILES: CC(=O)N1CCN(CCNC(=O)c2cnc(C)c(NC(=O)c3cnn4cc(-c5cnn(C)c5)sc34)c2)CC1]

To the mixture of 1-methyl-4-(4,4,5,5-tetramethyl-1,3,2-dioxaborolan-2-yl)pyrazole (50 mg, 0.08 mmol) and 1-methyl-4-(4,4,5,5-tetramethyl-1,3,2-dioxaborolan-2-yl)pyrazole (21 mg, 0.1 mmol) and K₃PO₄ (54 mg, 0.24 mmol), then was added Pd(dppf)₂C₁₂ (12 mg, 0.02 mmol) and dioxane (20 mL), H₂O (5 mL). After stirred at 90° C. 3 h, Then reaction mixture was concentrated under vacuum to give the crude product, which was purified by preparative high-performance liquid chromatography over column: Xtimate C18 100*30 mm*3 um to give the title compound N-(5-((2-(4-acetylpiperazin-1-yl)ethyl)carbamoyl)-2-methylpyridin-3-yl)-2-(1-methyl-1H-pyrazol-4-yl)pyrazolo[5,1-b]thiazole-7-carboxamide (30 mg, 64%) as a white solid. LCMS (ESI): mass calcd. for C₂₅H₂₉N₉O₃S, 535.6; m/z found, 536.4 [M+H]⁺. ¹H NMR (400 MHz, METHANOL-d₄) δ 8.79 (d, J=2.01 Hz, 1H), 8.43 (s, 1H), 8.34 (d, J=2.01 Hz, 1H), 8.26 (s, 1H), 8.05 (s, 1H), 7.85 (s, 1H), 3.97 (s, 3H), 3.56-3.66 (m, 6H), 2.67 (t, J=6.53 Hz, 2H), 2.63 (s, 3H), 2.58-2.62 (m, 2H), 2.55 (t, J=5.02 Hz, 2H) 2.11 (s, 3H).

### Example 130: 2-(6-aminopyridin-3-yl)-N-(5-((2-(2,2-dimethylpyrrolidin-1-yl)ethyl)carbamoyl)-2-methylpyridin-3-yl)pyrazolo[5,1-b]thiazole-7-carboxamide

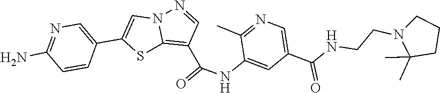 [SMILES: Cc1ncc(C(=O)NCCN2CCCC2(C)C)cc1NC(=O)c1cnn2cc(-c3ccc(N)nc3)sc12]

### Step a: 5-amino-6-methylnicotinic acid

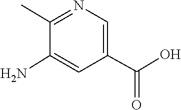

To a solution of sodium hydrate (1.11 g, 27.75 mmol) in ethanol (15 mL) was added ethyl 5-amino-6-methylnicotinate (5.00 g, 27.75 mmol) at room temperature. The reaction mixture was stirred at 50° C. for 15 mins before cooling to room temperature. The mixture was adjusted to pH=3-4 with HCl (aq, 2 M). The mixture was filtered and washed with water (10 mL×3). The solid was evaporated under vacuum to give desired product 5-amino-6-methylnicotinic acid (4.2 g, 99%) as a yellow solid. ¹H NMR (400 MHz, DMSO-d₆) δ 8.14 (d, J=1.54 Hz, 1H) 7.48 (d, J=1.76 Hz, 1H) 2.48 (br s, 2H), 2.33 (s, 3H)

### Step b: 5-amino-N-(2-(2,2-dimethylpyrrolidin-1-yl)ethyl)-6-methylnicotinamide

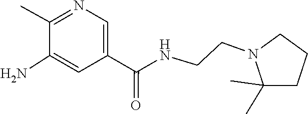 [SMILES: Cc1ncc(C(=O)NCCN2CCCC2(C)C)cc1N]

To a solution of 5-amino-6-methylnicotinic acid (5.4 g, 35.49 mmol), 2-(2,2-dimethylpyrrolidin-1-yl)ethanamine (5.05 g, 35.49 mmol) and N,N-diisopropylethylamine (18.35 g, 141.96 mmol) in DMF (50 mL) was added HATU (26.99 g, 70.98 mmol). The resulting mixture was stirred at 30° C. for 16 hours and then concentrated under vacuum to give the crude product, which was purified by preparative high-performance liquid chromatography over column: SANPONT C18, 250*80 mm*10 um, 100 A to give the title compound 5-amino-N-(2-(2,2-dimethylpyrrolidin-1-yl)ethyl)-6-methylnicotinamide (10 g, 68%) as a yellow solid. LCMS (ESI): mass calcd. for C₁₅H₂₄N₄O, 276.3; m/z found, 277.3 [M+H]⁺.

### Step c: 2-Bromopyrazolo[5,1-b]thiazole-7-carboxylic acid

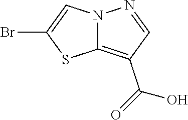 [SMILES: O=C(O)c1cnn2cc(Br)sc12]

To a solution of sodium hydrate (3.87 g, 7.75 mmol) in ethanol (10 mL) was added ethyl 2-bromopyrazolo[5,1-b]thiazole-7-carboxylate (3.1 g, 11.26 mmol) at room temperature. The reaction mixture was stirred at 40° C. for 16 h before cooling to room temperature. The mixture was adjusted to pH=3-4 with HCl (aq, 2 M). The mixture was filtered and washed with water (10 mL×3). The solid was evaporated under vacuum to give desired product 2-bromopyrazolo[5,1-b]thiazole-7-carboxylic acid (2.8 g, 97%) as white solid. LCMS (ESI): mass calcd. for C₆H₃BrN₂O₂S, 245.9; m/z found, 247 [M+H]⁺.

### Step d: 2-bromo-N-(5-((2-(2,2-dimethylpyrrolidin-1-yl)ethyl)carbamoyl)-2-methylpyridin-3-yl)pyrazolo[5,1-b]thiazole-7-carboxamide

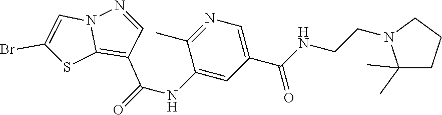 [SMILES: Cc1ncc(C(=O)NCCN2CCCC2(C)C)cc1NC(=O)c1cnn2cc(Br)sc12]

A mixture of 2-bromopyrazolo[5,1-b]thiazole-7-carboxylic acid (1 g, 4.04 mmol) in thionyl chloride (28 mL, 393 mmol) was stirred at 70° C. for 2 h. The reaction mixture was concentrated under vacuum to give the crude product as yellow solid 2-bromo-N-(5-((2-(2,2-dimethylpyrrolidin-1-yl)ethyl)carbamoyl)-2-methylpyridin-3-yl)pyrazolo[5,1-b]thiazole-7-carboxamide. 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (1 g, 3.61 mmol) was added to a solution consisting of ethyl 5-amino-6-methylnicotinate (1.487 g, 3.61 mmol), TEA(1.51 mL, 10.84 mmol) and THE (10 mL) at room temperature, The resulting mixture was stirred at 60° C. for 12 h. The resulting mixture was quenched with cooled H₂O and extracted with ethyl acetate (30 mL*3). The organic extracts were dried over anhydrous Na₂SO₄, and filtered, the filtrate was concentrated under reduced pressure to give crude product, which was purified by column chromatography over silica gel (eluent: dichloromethane/methyl alcohol=4:1) to give the title compound 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (1 g, 54.78%) as a yellow solid. LCMS (ESI): mass calcd. for C₂₁H₂₅BrN₆O₂S, 505.4; m/z found, 507.2 [M+H]⁺.

### Step e: 2-(6-aminopyridin-3-yl)-N-(5-((2-(2,2-dimethylpyrrolidin-1-yl)ethyl)carbamoyl)-2-methylpyridin-3-yl)pyrazolo[5,1-b]thiazole-7-carboxamide

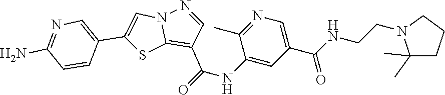 [SMILES: Cc1ncc(C(=O)NCCN2CCCC2(C)C)cc1NC(=O)c1cnn2cc(-c3ccc(N)nc3)sc12]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (80.0 mg, 0.16 mmol) and 5-(4,4,5,5-tetramethyl-1,3,2-dioxaborolan-2-yl)pyridin-2-amine (41.8 mg, 0.19 mmol) in 1,4-dioxane (4 mL) and H₂O (1 mL) was added [1,1-Bis(diphenylphosphino)ferrocene]palladium(II) chloride dichloromethane complex (12.9 mg, 0.02 mmol) and K₂CO₃ (65.63 mg, 0.48 mmol) under N₂. The resulting mixture was stirred at 95° C. for 12 h before cooled to 25° C. The reaction mixture was diluted with ethyl acetate (10 mL) and filtered over celite. The filtrate was concentrated to the crude product, which was treated with dichloromethane (10 mL) and water (10 mL). The organic phase was concentrated under reduced pressure to give the crude product, which was purified by preparative high-performance liquid chromatography over column: Boston Prime C18 150*30 mm*5 um to give the title compound 2-(6-aminopyridin-3-yl)-N-(5-((2-(2,2-dimethylpyrrolidin-1-yl)ethyl)carbamoyl)-2-methylpyridin-3-yl)pyrazolo[5,1-b]thiazole-7-carboxamide (15 mg, 17%) as a yellow oil. LCMS (ESI): mass calcd. for C₂₆H₃₀N₈O₂S, 532.6; m/z found, 533.3 [M+H]⁺. ¹H NMR (400 MHz, METHANOL-d₄) δ 8.78 (d, J=2.0 Hz, 1H), 8.42 (s, 1H), 8.33 (s, 1H), 8.31 (d, J=2.0 Hz, 1H), 8.22 (d, J=2.0 Hz, 1H), 7.76 (dd, J=2.5, 8.7 Hz, 1H), 6.67 (d, J=8.8 Hz, 1H), 3.56-3.49 (m, 2H), 2.90 (br t, J=7.5 Hz, 2H), 2.67 (t, J=6.9 Hz, 2H), 2.61 (s, 3H), 1.87-1.79 (m, 2H), 1.73-1.66 (m, 2H), 1.04 (s, 6H).

### Example 131: N-(5-((2-(2,2-dimethylpyrrolidin-1-yl)ethyl)carbamoyl)-2-methylpyridin-3-yl)-2-(2-methyl-2H-1,2,3-triazol-4-yl)pyrazolo[5,1-b]thiazole-7-carboxamide

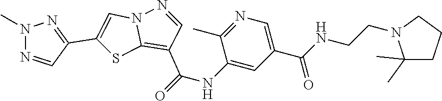 [SMILES: Cc1ncc(C(=O)NCCN2CCCC2(C)C)cc1NC(=O)c1cnn2cc(-c3cnn(C)n3)sc12]

To a solution of 4-bromo-2-methyl-2H-1,2,3-triazole (100 mg, 0.62 mmol), 4,4,4′,4′,5,5,5′,5′-octamethyl-2,2′-bi(1,3,2-dioxaborolane) (314 mg, 1.2 mmol) and potassium acetate (182 mg, 1.9 mmol) in dioxane (4 mL) was added Pd(dppf)Cl₂·CH₂Cl₂ (50.4 mg, 0.06 mmol) at room temperature. The resulting mixture was stirred at 60° C. overnight before cooling to room temperature. Then 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (156 mg, 0.31 mmol), K₂CO₃ (256 mg, 1.9 mmol), water (1 mL) and Pd(dppf)Cl₂·CH₂Cl₂ (50.4 mg, 0.06 mmol) was added. The reaction mixture was stirred at 60° C. overnight before cooling to room temperature. The reaction mixture was filtered through a pad of celite and the filtrate was concentrated under reduced pressure to give crude product, which was purified by preparative high-performance liquid chromatography over column: Boston Uni C18 40*150*5 um to give the title compound N-(5-((2-(2,2-dimethylpyrrolidin-1-yl)ethyl)carbamoyl)-2-methylpyridin-3-yl)-2-(2-methyl-2H-1,2,3-triazol-4-yl)pyrazolo[5,1-b]thiazole-7-carboxamide (12.6 mg, 4.0%) as a white solid. LCMS (ESI): mass calcd. for C₂₁H₂₆N₆O₂S, 507.6; m/z found, 508.2 [M+H]⁺. ¹H NMR (400 MHz, METHANOL-d₄) δ 8.83 (d, J=1.67 Hz, 1H), 8.60 (s, 1H), 8.49 (s, 1H), 8.35-8.42 (m, 1H), 8.10 (s, 1H), 4.24 (s, 3H), 3.54-3.85 (m, 4H), 3.34 (br s, 2H), 2.65 (s, 3H), 1.99-2.18 (m, 4H), 1.42 (s, 6H).

### Example 133: 4-(7-((5-((2-(2,2-dimethylpyrrolidin-1-yl)ethyl)carbamoyl)-2-methylpyridin-3-yl)carbamoyl)pyrazolo[5,1-b]thiazol-2-yl)benzoic acid

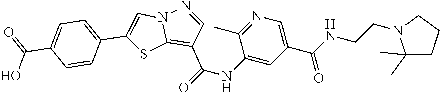 [SMILES: Cc1ncc(C(=O)NCCN2CCCC2(C)C)cc1NC(=O)c1cnn2cc(-c3ccc(C(=O)O)cc3)sc12]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (90.0 mg, 0.18 mmol) and 4-(4,4,5,5-tetramethyl-1,3,2-dioxaborolan-2-yl)benzoic acid (53.0 mg, 0.21 mmol) in 1,4-dioxane (3 mL) and H₂O (0.75 mL) was added [1,1-bis(diphenylphosphino)ferrocene]palladium(II) chloride dichloromethane complex (14.5 mg, 0.02 mmol) and K₂CO₃ (73.8 mg, 0.53 mmol) under N₂. The resulting mixture was stirred at 95° C. for 12 h before cooled to 25° C. The reaction mixture was diluted with ethyl acetate (10 mL) and filtered over celite. The filtrate was concentrated to the crude product, which was treated with dichloromethane (10 mL) and water (10 mL). The organic phase was concentrated under reduced pressure to give the crude product, which was purified by preparative high-performance liquid chromatography over column: Boston Prime C18 150*30 mm*5 um to give the title compound 4-(7-((5-((2-(2,2-dimethylpyrrolidin-1-yl)ethyl)carbamoyl)-2-methylpyridin-3-yl)carbamoyl)pyrazolo[5,1-b]thiazol-2-yl)benzoic acid (15 mg, 17%) as a yellow oil. LCMS (ESI): mass calcd. for C₂₈H₃₀N₆O₄S, 546.641; m/z found, 547.1 [M+H]⁺. ¹H NMR (400 MHz, METHANOL-d₄) δ 8.85 (d, J=2.0 Hz, 1H), 8.35 (d, J=2.0 Hz, 1H), 8.29 (s, 2H), 7.96 (d, J=8.4 Hz, 2H), 7.39 (br d, J=7.7 Hz, 2H), 3.85 (br s, 2H), 3.71-3.46 (m, 4H), 2.51 (s, 3H), 2.05 (br s, 4H), 1.39 (s, 6H).

### Example 134: 3-(7-((5-((2-(2,2-dimethylpyrrolidin-1-yl)ethyl)carbamoyl)-2-methylpyridin-3-yl)carbamoyl)pyrazolo[5,1-b]thiazol-2-yl)benzoic acid

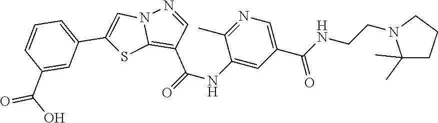 [SMILES: Cc1ncc(C(=O)NCCN2CCCC2(C)C)cc1NC(=O)c1cnn2cc(-c3cccc(C(=O)O)c3)sc12]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (60.0 mg, 0.12 mmol) and 3-(4,4,5,5-tetramethyl-1,3,2-dioxaborolan-2-yl)benzoic acid (53.0 mg, 0.21 mmol) in 1,4-dioxane (4 mL) and H₂O (1 mL) was added [1,1-bis(diphenylphosphino)ferrocene]palladium(II) chloride dichloromethane complex (9.7 mg, 0.01 mmol) and K₂CO₃ (49.2 mg, 0.36 mmol) under N₂. The resulting mixture was stirred at 95° C. for 12 h before cooled to 25° C. The reaction mixture was diluted with ethyl acetate (10 mL) and filtered over celite. The filtrate was concentrated to the crude product, which was treated with dichloromethane (10 mL) and water (10 mL). The organic phase was concentrated under reduced pressure to give the crude product, which was purified by preparative high-performance liquid chromatography over column: Boston Prime C18 150*30 mm*5 um to give the title compound 3-(7-((5-((2-(2,2-dimethylpyrrolidin-1-yl)ethyl)carbamoyl)-2-methylpyridin-3-yl)carbamoyl)pyrazolo[5,1-b]thiazol-2-yl)benzoic acid (19 mg, 28%) as a yellow oil. LCMS (ESI): mass calcd. for C₂₈H₃₀N₆O₄S, 546.641; m/z found, 547.2 [M+H]⁺. ¹H NMR (400 MHz, METHANOL-d₄) δ 8.78 (d, J=2.0 Hz, 1H), 8.45 (s, 1H), 8.37 (d, J=1.9 Hz, 1H), 8.34 (s, 1H), 8.22 (s, 1H), 7.96 (d, J=7.9 Hz, 1H), 7.67 (br d, J=8.5 Hz, 1H), 7.44 (t, J=7.7 Hz, 1H), 3.68-3.62 (m, 2H), 3.19 (br s, 2H), 2.93 (br s, 2H), 2.59 (s, 3H), 1.92 (br d, J=7.6 Hz, 2H), 1.84 (br d, J=8.3 Hz, 2H), 1.19 (s, 6H).

### Example 135: N-(5-((2-(2,2-dimethylpyrrolidin-1-yl)ethyl)carbamoyl)-2-methylpyridin-3-yl)-2-(pyridin-3-yl)pyrazolo[5,1-b]thiazole-7-carboxamide

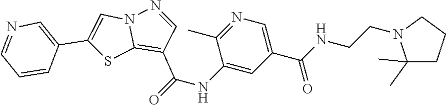 [SMILES: Cc1ncc(C(=O)NCCN2CCCC2(C)C)cc1NC(=O)c1cnn2cc(-c3cccnc3)sc12]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (70.0 mg, 0.14 mmol) and pyridin-3-ylboronic acid (20.4 mg, 0.17 mmol) in 1,4-dioxane (4 mL) and H₂O (1 mL) was added [1,1-Bis(diphenylphosphino)ferrocene]palladium(II) chloride dichloromethane complex (11.3 mg, 0.01 mmol) and K₃PO₄ (88.2 mg, 0.42 mmol) under N₂. The resulting mixture was stirred at 95° C. for 12 h before cooled to 25° C. The reaction mixture was diluted with ethyl acetate (10 mL) and filtered over celite. The filtrate was concentrated to the crude product, which was treated with dichloromethane (10 mL) and water (10 mL). The organic phase was concentrated under reduced pressure to give the crude product, which was purified by preparative high-performance liquid chromatography over column: Boston Prime C18 150*30 mm*5 um to give the title compound N-(5-((2-(2,2-dimethylpyrrolidin-1-yl)ethyl)carbamoyl)-2-methylpyridin-3-yl)-2-(pyridin-3-yl)pyrazolo[5,1-b]thiazole-7-carboxamide (14 mg, 19%) as a yellow oil. LCMS (ESI): mass calcd. for C₂₆H₂₉N₇O₂S, 503.619; m/z found, 547.2 [M+H]⁺. ¹H NMR (400 MHz, METHANOL-d₄) δ 8.93 (d, J=1.8 Hz, 1H), 8.80 (d, J=1.8 Hz, 1H), 8.72 (s, 1H), 8.59 (d, J=4.3 Hz, 1H), 8.50 (s, 1H), 8.34 (s, 1H), 8.17 (br d, J=8.0 Hz, 1H), 7.57 (dd, J=4.8, 8.3 Hz, 1H), 3.59 (br s, 2H), 3.11 (br d, J=18.6 Hz, 2H), 2.84 (br s, 2H), 2.63 (s, 3H), 1.91 (br s, 2H), 1.79 (br s, 2H), 1.14 (br s, 6H).

### Example 136: N-(5-((2-(2,2-dimethylpyrrolidin-1-yl)ethyl)carbamoyl)-2-methylpyridin-3-yl)-2-(6-methylpyridin-3-yl)pyrazolo[5,1-b]thiazole-7-carboxamide

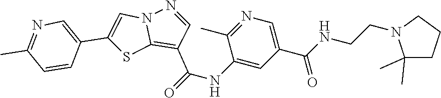 [SMILES: Cc1ccc(-c2cn3ncc(C(=O)Nc4cc(C(=O)NCCN5CCCC5(C)C)cnc4C)c3s2)cn1]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (70.0 mg, 0.14 mmol) and 2-methyl-5-(4,4,5,5-tetramethyl-1,3,2-dioxaborolan-2-yl)pyridine (36.4 mg, 0.17 mmol) in 1,4-dioxane (3 mL) and H₂O (0.75 mL) was added [1,1-bis(diphenylphosphino)ferrocene]palladium(II) chloride dichloromethane complex (11.3 mg, 0.01 mmol) and NaHCO₃ (34.9 mg, 0.42 mmol) under N₂. The resulting mixture was stirred at 95° C. for 12 h before cooled to 25° C. The reaction mixture was diluted with ethyl acetate (10 mL) and filtered over celite. The filtrate was concentrated to the crude product, which was treated with dichloromethane (10 mL) and water (10 mL). The organic phase was concentrated under reduced pressure to give the crude product, which was purified by preparative high-performance liquid chromatography over column: Boston Prime C18 150*30 mm*5 um to give the title compound N-(5-((2-(2,2-dimethylpyrrolidin-1-yl)ethyl)carbamoyl)-2-methylpyridin-3-yl)-2-(6-methylpyridin-3-yl)pyrazolo[5,1-b]thiazole-7-carboxamide (13 mg, 18%) as a yellow oil. LCMS (ESI): mass calcd. for C₂₇H₃₁N₇O₂S, 517.646; m/z found, 518.4 [M+H]⁺. ¹H NMR (400 MHz, METHANOL-d₄) δ 8.80 (br s, 2H), 8.66 (s, 1H), 8.49 (s, 1H), 8.33 (s, 1H), 8.06 (br d, J=7.8 Hz, 1H), 7.45 (d, J=8.0 Hz, 1H), 3.54 (br t, J=6.9 Hz, 2H), 2.91 (br d, J=7.5 Hz, 2H), 2.70 (br t, J=6.9 Hz, 2H), 2.63 (s, 3H), 2.61 (s, 3H), 1.90-1.81 (m, 2H), 1.75-1.68 (m, 2H), 1.07 (s, 6H).

### Example 137: N-(5-((2-(2,2-dimethylpyrrolidin-1-yl)ethyl)carbamoyl)-2-methylpyridin-3-yl)-2-(6-oxo-1,6-dihydropyridin-3-yl)pyrazolo[5,1-b]thiazole-7-carboxamide

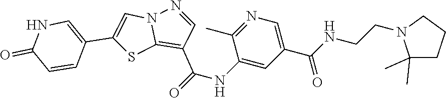 [SMILES: Cc1ncc(C(=O)NCCN2CCCC2(C)C)cc1NC(=O)c1cnn2cc(-c3ccc(=O)[nH]c3)sc12]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (50.0 mg, 0.10 mmol) and 5-(4,4,5,5-tetramethyl-1,3,2-dioxaborolan-2-yl)pyridin-2 (1H)-one (26.2 mg, 0.12 mmol) in 1,4-dioxane (3 mL) and H₂O (0.75 mL) was added [1,1-Bis(diphenylphosphino)ferrocene]palladium(II) chloride dichloromethane complex (8.1 mg, 0.01 mmol) and K₃PO₄ (62.9 mg, 0.30 mmol) under N₂. The resulting mixture was stirred at 95° C. for 12 h before cooled to 25° C. The reaction mixture was diluted with ethyl acetate (10 mL) and filtered over celite. The filtrate was concentrated to the crude product, which was treated with dichloromethane (10 mL) and water (10 mL). The organic phase was concentrated under reduced pressure to give the crude product, which was purified by preparative high-performance liquid chromatography over column: Boston Prime C18 150*30 mm*5 um to give the title compound N-(5-((2-(2,2-dimethylpyrrolidin-1-yl)ethyl)carbamoyl)-2-methylpyridin-3-yl)-2-(6-oxo-1,6-dihydropyridin-3-yl)pyrazolo[5,1-b]thiazole-7-carboxamide (14 mg, 27%) as a yellow oil. LCMS (ESI): mass calcd. for C₂₆H₂₉N₇O₃S, 519.619; m/z found, 520.3 [M+H]⁺. ¹H NMR (400 MHz, METHANOL-d₄) δ 8.80 (s, 1H), 8.49-8.32 (m, 3H), 7.95 (br d, J=9.8 Hz, 1H), 7.83 (s, 1H), 6.68 (d, J=9.8 Hz, 1H), 3.58 (br s, 2H), 3.02 (br s, 2H), 2.79 (br s, 2H), 2.63 (s, 3H), 1.90 (br s, 2H), 1.76 (br s, 2H), 1.11 (s, 6H).

### Example 138: N-(5-((2-(2,2-dimethylpyrrolidin-1-yl)ethyl)carbamoyl)-2-methylpyridin-3-yl)-2-(1-methyl-6-oxo-1,6-dihydropyridin-3-yl)pyrazolo[5,1-b]thiazole-7-carboxamide

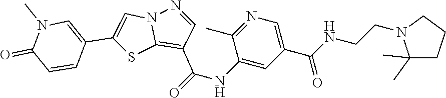 [SMILES: Cc1ncc(C(=O)NCCN2CCCC2(C)C)cc1NC(=O)c1cnn2cc(-c3ccc(=O)n(C)c3)sc12]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (50.0 mg, 0.10 mmol) and 1-methyl-5-(4,4,5,5-tetramethyl-1,3,2-dioxaborolan-2-yl)pyridin-2 (1H)-one (27.9 mg, 0.12 mmol) in 1,4-dioxane (3 mL) and H₂O (0.75 mL) was added [1,1-Bis(diphenylphosphino)ferrocene]palladium(II) chloride dichloromethane complex (8.1 mg, 0.01 mmol) and K₃PO₄ (62.9 mg, 0.29 mmol) under N₂. The resulting mixture was stirred at 95° C. for 12 h before cooled to 25° C. The reaction mixture was diluted with ethyl acetate (10 mL) and filtered over celite. The filtrate was concentrated to the crude product, which was treated with dichloromethane (10 mL) and water (10 mL). The organic phase was concentrated under reduced pressure to give the crude product, which was purified by preparative high-performance liquid chromatography over column: Boston Prime C18 150*30 mm*5 um to give the title compound N-(5-((2-(2,2-dimethylpyrrolidin-1-yl)ethyl)carbamoyl)-2-methylpyridin-3-yl)-2-(1-methyl-6-oxo-1,6-dihydropyridin-3-yl)pyrazolo[5,1-b]thiazole-7-carboxamide (18 mg, 34%) as a yellow oil. LCMS (ESI): mass calcd. for C₂₇H₃₁N₇O₃S, 533.645; m/z found, 534.3 [M+H]⁺. ¹H NMR (400 MHz, METHANOL-d₄) δ 8.80 (s, 1H), 8.46 (s, 1H), 8.40 (s, 1H), 8.33 (br s, 1H), 8.14 (br s, 1H), 7.90 (br d, J=10.5 Hz, 1H), 6.68 (br d, J=8.5 Hz, 1H), 3.66 (s, 3H), 3.54 (br s, 2H), 2.93 (br s, 2H), 2.70 (br s, 2H), 2.63 (s, 3H), 1.85 (br s, 2H), 1.72 (br d, J=7.5 Hz, 2H), 1.07 (br s, 6H).

### Example 139: N-(5-((2-(2,2-dimethylpyrrolidin-1-yl)ethyl)carbamoyl)-2-methylpyridin-3-yl)-2-(pyrimidin-5-yl)pyrazolo[5,1-b]thiazole-7-carboxamide

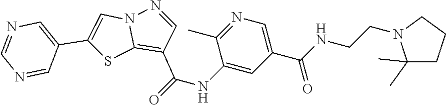 [SMILES: Cc1ncc(C(=O)NCCN2CCCC2(C)C)cc1NC(=O)c1cnn2cc(-c3cncnc3)sc12]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (150 mg, 0.30 mmol) and 5-(4,4,5,5-tetramethyl-1,3,2-dioxaborolan-2-yl)pyrimidine (73.4 mg, 0.36 mmol) in 1,4-dioxane (3 mL) and H₂O (0.75 mL) was added [1,1-bis(diphenylphosphino)ferrocene]palladium(II) chloride dichloromethane complex (24.2 mg, 0.03 mmol) and NaHCO₃ (74.8 mg, 0.29 mmol) under N₂. The resulting mixture was stirred at 95° C. for 12 h before cooled to 25° C. The reaction mixture was diluted with ethyl acetate (10 mL) and filtered over celite. The filtrate was concentrated to the crude product, which was treated with dichloromethane (10 mL) and water (10 mL). The organic phase was concentrated under reduced pressure to give the crude product, which was purified by preparative high-performance liquid chromatography over column: Boston Prime C18 150*30 mm*5 um to give the title compound N-(5-((2-(2,2-dimethylpyrrolidin-1-yl)ethyl)carbamoyl)-2-methylpyridin-3-yl)-2-(pyrimidin-5-yl)pyrazolo[5,1-b]thiazole-7-carboxamide (16 mg, 10%) as a yellow oil. LCMS (ESI): mass calcd. for C₂₅H₂₈N₈O₂S, 504.607; m/z found, 505.3 [M+H]⁺. ¹H NMR (400 MHz, METHANOL-d₄) δ 9.18 (s, 1H), 9.14 (s, 2H), 8.81 (br d, J=2.3 Hz, 2H), 8.51 (br s, 0.63H), 8.51-8.48 (m, 1H), 8.38 (s, 1H), 3.76 (br t, J=5.9 Hz, 2H), 3.57 (br s, 2H), 3.27 (br s, 2H), 2.63 (s, 3H), 2.17-2.06 (m, 2H), 2.06-1.97 (m, 2H), 1.37 (s, 6H).

### Example 140: N-(5-((2-(2,2-dimethylpyrrolidin-1-yl)ethyl)carbamoyl)-2-methylpyridin-3-yl)-2-(1-(2-hydroxyethyl)-1H-pyrazol-4-yl)pyrazolo[5,1-b]thiazole-7-carboxamide

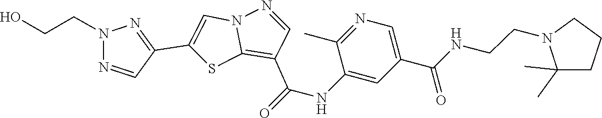 [SMILES: Cc1ncc(C(=O)NCCN2CCCC2(C)C)cc1NC(=O)c1cnn2cc(-c3cnn(CCO)n3)sc12]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (150 mg, 0.30 mmol) and 2-(4-(4,4,5,5-tetramethyl-1,3,2-dioxaborolan-2-yl)-1H-pyrazol-1-yl)ethanol (84.8 mg, 0.36 mmol) in 1,4-dioxane (3 mL) and H₂O (0.75 mL) was added [1,1-bis(diphenylphosphino)ferrocene]palladium(II) chloride dichloromethane complex (24.2 mg, 0.03 mmol) and K₃PO₄ (188 mg, 0.89 mmol) under N₂. The resulting mixture was stirred at 90° C. for 3 h before cooled to 25° C. The reaction mixture was diluted with ethyl acetate (10 mL) and filtered over celite. The filtrate was concentrated to the crude product, which was treated with dichloromethane (10 mL) and water (10 mL). The organic phase was concentrated under reduced pressure to give the crude product, which was purified by preparative high-performance liquid chromatography over column: Boston Prime C18 150*30 mm*5 um to give the title compound N-(5-((2-(2,2-dimethylpyrrolidin-1-yl)ethyl)carbamoyl)-2-methylpyridin-3-yl)-2-(1-(2-hydroxyethyl)-1H-pyrazol-4-yl)pyrazolo[5,1-b]thiazole-7-carboxamide (41.5 mg, 25%) as a yellow oil. LCMS (ESI): mass calcd. for C₂₆H₃₂N₈O₃S, 536.649; m/z found, 537.4 [M+H]⁺. ¹H NMR (400 MHz, METHANOL-d₄) δ 8.78 (br s, 1H), 8.42 (br s, 1H), 8.33 (br s, 1H), 8.25 (s, 1H), 8.10 (s, 1H), 7.88 (s, 1H), 4.29 (br s, 2H), 3.94 (br s, 2H), 3.55 (br s, 2H), 2.96 (br s, 2H), 2.73 (br s, 2H), 2.62 (s, 3H), 1.86 (br s, 2H), 1.74 (br d, J=7.5 Hz, 2H), 1.08 (br s, 6H).

### Example 141: N-(5-((2-(2,2-dimethylpyrrolidin-1-yl)ethyl)carbamoyl)-2-methylpyridin-3-yl)-2-(6-oxo-1,6-dihydropyridazin-3-yl)pyrazolo[5,1-b]thiazole-7-carboxamide

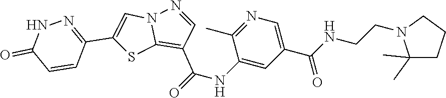 [SMILES: Cc1ncc(C(=O)NCCN2CCCC2(C)C)cc1NC(=O)c1cnn2cc(-c3ccc(=O)[nH]n3)sc12]

### Step a: 6-chloro-2-((2-(trimethylsilyl)ethoxy)methyl)pyridazin-3 (2H)-one

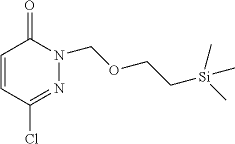 [SMILES: C[Si](C)(C)CCOCn1nc(Cl)ccc1=O]

To a solution of sodium hydride (919 mg, 22.9 mmol) in THE (30 mL) was added 6-chloropyridazin-3 (2H)-one (2.5 g, 19.1 mmol) at 0° C. The mixture was stirred at room temperature for 30 mins. Then (2-(chloromethoxy)ethyl)trimethylsilane (3.6 mL, 20.1 mmol) was added dropwise at 0° C. The resulting mixture was stirred at room temperature for 12 h. The resulting mixture was quenched with cooled H₂O and extracted with ethyl acetate (20 mL*3). The organic extracts were dried over anhydrous Na₂SO₄, and filtered, the filtrate was concentrated under reduced pressure to give crude product, which was purified by column chromatography over silica gel (eluent: petroleum ether/ethyl acetate=3:2) to give the title compound 6-chloro-2-((2-(trimethylsilyl)ethoxy)methyl)pyridazin-3 (2H)-one (1.5 g, 30%) as a yellow oil.

### Step b: N-(5-((2-(2,2-dimethylpyrrolidin-1-yl)ethyl)carbamoyl)-2-methylpyridin-3-yl)-2-(6-oxo-1-((2-(trimethylsilyl)ethoxy)methyl)-1,6-dihydropyridazin-3-yl)pyrazolo[5,1-b]thiazole-7-carboxamide

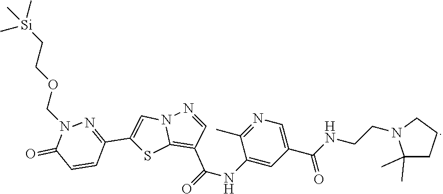 [SMILES: Cc1ncc(C(=O)NCCN2CCCC2(C)C)cc1NC(=O)c1cnn2cc(-c3ccc(=O)n(COCC[Si](C)(C)C)n3)sc12]

To a solution of 6-chloro-2-((2-(trimethylsilyl)ethoxy)methyl)pyridazin-3 (2H)-one (247 mg, 0.95 mmol), 4,4,4′,4′,5,5,5′,5′-octamethyl-2,2′-bi(1,3,2-dioxaborolane) (261 mg, 1.0 mmol), potassium acetate (503 mg, 2.4 mmol) in dioxane (1.5 mL) was added diacetoxypalladium (17.7 mg, 0.08 mmol) and dicyclohexyl(2′,4′,6′-triisopropyl-[1,1′-biphenyl]-2-yl)phosphine (37.7 mg, 0.08 mmol) at room temperature. The mixture was stirred at 95° C. for 12 h. Then 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (400 mg, 0.79 mmol), Pd(dppf)Cl₂·DCM (96.9 mg, 0.12 mmol), potassium phosphate (504 mg, 2.4 mmol) and H₂O (6 mL) were added dropwise at room temperature. The resulting mixture was stirred at 95° C. for 4 h. The resulting mixture was quenched with cooled H₂O and extracted with ethyl acetate (20 mL*3). The organic extracts were dried over anhydrous Na₂SO₄, and filtered, the filtrate was concentrated under reduced pressure to give crude product, which was purified by column chromatography over silica gel (eluent: petroleum ether/ethyl acetate=7:3) to give the title compound N-(5-((2-(2,2-dimethylpyrrolidin-1-yl)ethyl)carbamoyl)-2-methylpyridin-3-yl)-2-(6-oxo-1-((2-(trimethylsilyl)ethoxy)methyl)-1,6-dihydropyridazin-3-yl)pyrazolo[5,1-b]thiazole-7-carboxamide (200 mg, 39%) as a yellow solid. LCMS (ESI): mass calcd. for C₃₁H₄₂N₈O₄SSi, 650.867; m/z found, 651.4 [M+H]⁺.

### Step c: N-(5-((2-(2,2-dimethylpyrrolidin-1-yl)ethyl)carbamoyl)-2-methylpyridin-3-yl)-2-(6-oxo-1,6-dihydropyridazin-3-yl)pyrazolo[5,1-b]thiazole-7-carboxamide

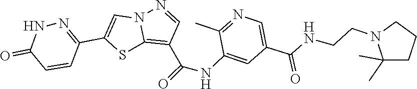 [SMILES: Cc1ncc(C(=O)NCCN2CCCC2(C)C)cc1NC(=O)c1cnn2cc(-c3ccc(=O)[nH]n3)sc12]

To a solution of N-(5-((2-(2,2-dimethylpyrrolidin-1-yl)ethyl)carbamoyl)-2-methylpyridin-3-yl)-2-(6-oxo-1-((2-(trimethylsilyl)ethoxy)methyl)-1,6-dihydropyridazin-3-yl)pyrazolo[5,1-b]thiazole-7-carboxamide (100 mg, 0.154 mmol) in dichloromethane (1 mL) was added trifluoroacetic acid (1 mL, 13.5 mmol). The mixture stirred at 25° C. for 12 h and then concentrated under vacuum to give the crude product. The crude product was purified by preparative high-performance liquid chromatography over column: Boston Green ODS 150*30 mm*5 um to give the title compound N-(5-((2-(2,2-dimethylpyrrolidin-1-yl)ethyl)carbamoyl)-2-methylpyridin-3-yl)-2-(6-oxo-1,6-dihydropyridazin-3-yl)pyrazolo[5,1-b]thiazole-7-carboxamide (18 mg, 22%) as a white solid. LCMS (ESI): mass calcd. for C₂₅H₂₈N₈O₃S, 520.607; m/z found, 521.3 [M+H]⁺. ¹H NMR (400 MHz, METHANOL-d₄) δ 8.83 (d, J=2.0 Hz, 1H), 8.80 (s, 1H), 8.51 (s, 1H), 8.39 (s, 1H), 8.08 (d, J=10.0 Hz, 1H), 7.11 (d, J=10.0 Hz, 1H), 3.75 (br s, 2H), 3.31-3.16 (m, 4H), 2.65 (s, 3H), 2.15-2.00 (m, 4H), 1.37 (br s, 6H).

### Example 142: N-(5-((2-(2-azabicyclo[2.2.2]octan-2-yl)ethyl)carbamoyl)-2-methylpyridin-3-yl)-2-(1-(2-methoxyethyl)-1H-pyrazol-4-yl)pyrazolo[5,1-b]thiazole-7-carboxamide

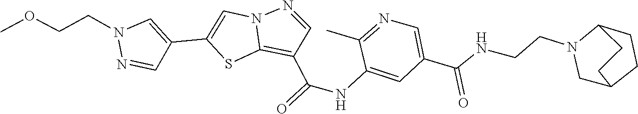 [SMILES: COCCn1cc(-c2cn3ncc(C(=O)Nc4cc(C(=O)NCCN5CC6CCC5CC6)cnc4C)c3s2)cn1]

### Step a: N-(5-((2-(2-azabicyclo[2.2.2]octan-2-yl)ethyl)carbamoyl)-2-methylpyridin-3-yl)-2-bromopyrazolo[5,1-b]thiazole-7-carboxamide

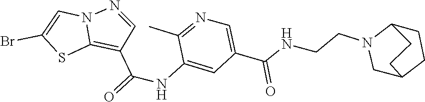 [SMILES: Cc1ncc(C(=O)NCCN2CC3CCC2CC3)cc1NC(=O)c1cnn2cc(Br)sc12]

To a solution of 5-(2-bromopyrazolo[5,1-b]thiazole-7-carboxamido)-6-methylnicotinic acid (3.6 g, 9.2 mmol), DIEA (6.0 mL, 37 mmol) and 2-(2-azabicyclo[2.2.2]octan-2-yl)ethanamine (1.7 g, 11 mmol) in DMF (22 mL) was added HATU (5.2 g, 14 mmol) slowly. The resulting mixture was stirred at room temperature for 1 h and then poured into water. The mixture was filtered and give the product. The product concentrated under vacuum to give title compound N-(5-((2-(2-azabicyclo[2.2.2]octan-2-yl)ethyl)carbamoyl)-2-methylpyridin-3-yl)-2-bromopyrazolo[5,1-b]thiazole-7-carboxamide (10 g, 67%) as a white solid. LCMS (ESI): mass calcd. for C₂₂H₂₅Br₂N₆O₂S, 517.4; m/z found, 518.8 [M+H]⁺.

### Step b: N-(5-((2-(2-azabicyclo[2.2.2]octan-2-yl)ethyl)carbamoyl)-2-methylpyridin-3-yl)-2-(1-(2-methoxyethyl)-1H-pyrazol-4-yl)pyrazolo[5,1-b]thiazole-7-carboxamide

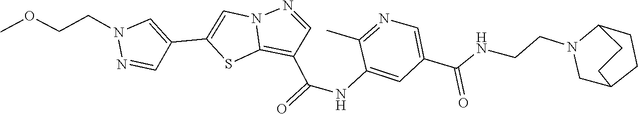 [SMILES: COCCn1cc(-c2cn3ncc(C(=O)Nc4cc(C(=O)NCCN5CC6CCC5CC6)cnc4C)c3s2)cn1]

To a solution of N-(5-((2-(2-azabicyclo[2.2.2]octan-2-yl)ethyl)carbamoyl)-2-methylpyridin-3-yl)-2-bromopyrazolo[5,1-b]thiazole-7-carboxamide (150 mg, 0.29 mmol), Cs₂CO₃ (283 mg, 0.87 mmol) and 1-(2-methoxyethyl)-4-(4,4,5,5-tetramethyl-1,3,2-dioxaborolan-2-yl)-1H-pyrazole (88 mg, 0.35 mmol) in dioxane:H₂O=4:1 (20 mL) was Pd(dppf)Cl₂·CH₂Cl₂ (71 mg, 0.087 mmol). The resulting mixture was stirred at 100° C. for 12 hours. The mixture was filtered through a celite pad and washed with dichloromethane (10 mL×3), then the filter was evaporated under vacuum to give residue, which was purified by column chromatography over silica gel (eluent: Petroleum ether/ethyl acetate=0:100 to 100:0 then ethyl acetate/methanol=90:10) to give the crude product. The crude product was purified by preparative high-performance liquid chromatography over column: Phenomenex Gemini-NX C18 75*30 mm*3 um to give title compound N-(5-((2-(2-azabicyclo[2.2.2]octan-2-yl)ethyl)carbamoyl)-2-methylpyridin-3-yl)-2-(1-(2-methoxyethyl)-1H-pyrazol-4-yl)pyrazolo[5,1-b]thiazole-7-carboxamide (54.3 mg, 30%) as a white solid. LCMS (ESI): mass calcd. for C₂₈H₃₄N₈O₃S, 562.7; m/z found, 563.3 [M+H]⁺. ¹H NMR (400 MHz, METHANOL-d₄) δ 8.82 (d, J=1.3 Hz, 1H), 8.54 (br s, 1H), 8.44 (s, 1H), 8.39 (d, J=1.3 Hz, 1H), 8.27 (s, 1H), 8.08 (s, 1H), 7.86 (s, 1H), 4.37 (t, J=5.1 Hz, 2H), 3.78 (q, J=4.7 Hz, 4H), 3.59 (br s, 1H), 3.53-3.38 (m, 4H), 3.36 (s, 3H), 2.64 (s, 3H), 2.19 (br s, 2H), 2.01 (br s, 1H), 1.93-1.74 (m, 6H).

### Example 143: N-(5-((2-(2-azabicyclo[2.2.2]octan-2-yl)ethyl)carbamoyl)-2-methylpyridin-3-yl)-2-(1-((methylsulfonyl)methyl)-1H-pyrazol-4-yl)pyrazolo[5,1-b]thiazole-7-carboxamide

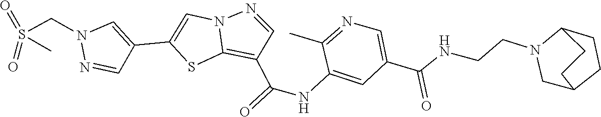 [SMILES: Cc1ncc(C(=O)NCCN2CC3CCC2CC3)cc1NC(=O)c1cnn2cc(-c3cnn(CS(C)(=O)=O)c3)sc12]

To a solution of 1-(methylsulfonylmethyl)-4-(4,4,5,5-tetramethyl-1,3,2-dioxaborolan-2-yl)pyrazole (200 mg, 0.39 mmol), 1-((methylsulfonyl)methyl)-4-(4,4,5,5-tetramethyl-1,3,2-dioxaborolan-2-yl)-1H-pyrazole (144 mg, 0.51 mmol) and Na₂CO₃ (123 mg, 1.2 mmol) in dioxane:H₂O=4:1 (12 mL) was added PdCl₂(dppf)·CH₂Cl₂ (95 mg, 0.12 mmol) at room temperature. The resultant mixture was stirred at 100° C. for 3 h. Then the mixture was concentrated and triturated with dichloromethane (10 mL×3) and then concentrated under vacuum to give the crude product, which was purified by preparative high-performance liquid chromatography over column: Phenomenex Gemini-NX C18 75*30 mm*3 um to give the title compound N-(5-((2-(2-azabicyclo[2.2.2]octan-2-yl)ethyl)carbamoyl)-2-methylpyridin-3-yl)-2-(1-((methylsulfonyl)methyl)-1H-pyrazol-4-yl)pyrazolo[5,1-b]thiazole-7-carboxamide (62 mg, 42%) as a white solid. LCMS (ESI): mass calcd. for C₂₇H₃₂N₈O₄S₂, 596.7; m/z found, 597.3 [M+H]⁺. ¹H NMR (400 MHz, METHANOL-d₄) δ 8.78 (d, J=2.0 Hz, 1H), 8.81-8.77 (m, 1H), 8.49 (br s, 1H), 8.41 (s, 1H), 8.35 (d, J=2.0 Hz, 1H), 8.32 (s, 1H), 8.23 (s, 1H), 8.00 (s, 1H), 5.64 (s, 2H), 3.73 (t, J=6.0 Hz, 2H), 3.54 (br s, 1H), 3.41 (t, J=6.1 Hz, 2H), 3.35 (br s, 2H), 3.00 (s, 3H), 2.60 (s, 3H), 2.14 (br d, J=5.1 Hz, 2H), 1.97 (br s, 1H), 1.88-1.72 (m, 6H).

### Example 144: N-(5-((2-(2-azabicyclo[2.2.2]octan-2-yl)ethyl)carbamoyl)-2-methylpyridin-3-yl)-2-(1-(oxetan-3-yl)-1H-pyrazol-4-yl)pyrazolo[5,1-b]thiazole-7-carboxamide

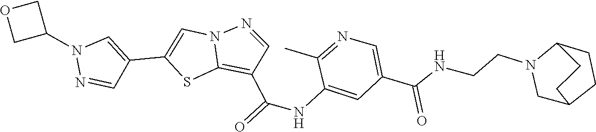 [SMILES: Cc1ncc(C(=O)NCCN2CC3CCC2CC3)cc1NC(=O)c1cnn2cc(-c3cnn(C4COC4)c3)sc12]

To a solution of N-(5-((2-(2-azabicyclo[2.2.2]octan-2-yl)ethyl)carbamoyl)-2-methylpyridin-3-yl)-2-(1-bromo-1H-pyrazol-4-yl)pyrazolo[5,1-b]thiazole-7-carboxamide (150 mg, 0.29 mmol), 1-(methylsulfonylmethyl)-4-(4,4,5,5-tetramethyl-1,3,2-dioxaborolan-2-yl)pyrazole (94 mg, 0.38 mmol) and Na₂CO₃ (92 mg, 0.87 mmol) in dioxane:H₂O=4:1 (18 mL) was added PdCl₂(dppf)·CH₂Cl₂ (71 mg, 0.09 mmol) at room temperature. The resultant mixture was stirred at 100° C. for 3 h. Then the mixture was concentrated and triturated with dichloromethane (10 mL×3) and then concentrated under vacuum to give the crude product, which was purified by preparative high-performance liquid chromatography over column: Phenomenex Gemini-NX C18 75*30 mm*3 um to give the title compound N-(5-((2-(2-azabicyclo[2.2.2]octan-2-yl)ethyl)carbamoyl)-2-methylpyridin-3-yl)-2-(1-(oxetan-3-yl)-1H-pyrazol-4-yl)pyrazolo[5,1-b]thiazole-7-carboxamide (48 mg, 26%) as a white solid. LCMS (ESI): mass calcd. for C₂₈H₃₂N₈O₃S, 560.7; m/z found, 561.3, [M+H]⁺. ¹H NMR (400 MHz, METHANOL-d₄) δ 8.64 (s, 1H), 8.32 (br s, 1H), 8.26 (s, 1H), 8.21 (s, 1H), 8.08 (s, 1H), 8.04 (s, 1H), 7.79 (s, 1H), 5.45 (quin, J=6.8 Hz, 1H), 4.97-4.85 (m, 4H), 3.62 (br s, 2H), 3.43 (br s, 2H), 3.25 (br s, 2H), 3.17 (br s, 1H), 2.46 (s, 3H), 2.04 (br s, 2H), 1.84 (br s, 1H), 1.77-1.51 (m, 6H).

### Example 145: N-(5-((2-(2-azabicyclo[2.2.2]octan-2-yl)ethyl)carbamoyl)-2-methylpyridin-3-yl)-2-(2-oxo-2,3-dihydrobenzo[d]oxazol-5-yl)pyrazolo[5,1-b]thiazole-7-carboxamide

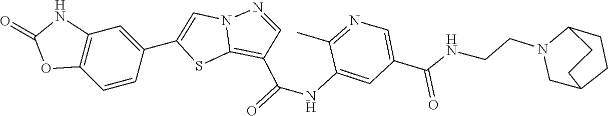 [SMILES: Cc1ncc(C(=O)NCCN2CC3CCC2CC3)cc1NC(=O)c1cnn2cc(-c3ccc4oc(=O)[nH]c4c3)sc12]

To a solution of N-(5-((2-(2-azabicyclo[2.2.2]octan-2-yl)ethyl)carbamoyl)-2-methylpyridin-3-yl)-2-bromopyrazolo[5,1-b]thiazole-7-carboxamide (300 mg, 0.58 mmol), 5-(4,4,5,5-tetramethyl-1,3,2-dioxaborolan-2-yl)benzo[d]oxazol-2 (3H)-one (1 g, 1.1 mmol) and Na₂CO₃ (185 mg, 1.7 mmol) in dioxane:H₂O=4:1 (20 mL) was added PdCl₂(dppf)·CH₂Cl₂ (146 mg, 0.18 mmol) at room temperature. The resultant mixture was stirred at 100° C. for 12 h. Then the mixture was concentrated and triturated with dichloromethane (10 mL×3) and then concentrated under vacuum to give the crude product, which was purified by preparative high-performance liquid chromatography over column: Boston Green ODS 150*30 mm*5 um to give the title compound N-(5-((2-(2-azabicyclo[2.2.2]octan-2-yl)ethyl)carbamoyl)-2-methylpyridin-3-yl)-2-(2-oxo-2,3-dihydrobenzo[d]oxazol-5-yl)pyrazolo[5,1-b]thiazole-7-carboxamide (16.9 mg, 4.7%) as a white solid. LCMS (ESI): mass calcd. for C₂₉H₂₉N₇O₄S, 617.7; m/z found, 572.3, [M+H]⁺. ¹H NMR (400 MHz, METHANOL-d₄) δ 8.81 (d, J=1.7 Hz, 1H), 8.51 (br s, 1H), 8.49 (s, 1H), 8.46 (s, 1H), 8.38 (s, 1H), 7.45 (d, J=8.3 Hz, 1H), 7.42 (s, 1H), 7.33 (d, J=8.3 Hz, 1H), 3.75 (t, J=6.0 Hz, 2H), 3.55 (br s, 1H), 3.41 (br t, J=6.1 Hz, 2H), 3.36 (br s, 2H), 2.64 (s, 3H), 2.15 (br d, J=8.8 Hz, 2H), 1.99 (br s, 1H), 1.89-1.75 (m, 6H).

### Example 146: N-(5-((2-(2-azabicyclo[2.2.2]octan-2-yl)ethyl)carbamoyl)-2-methylpyridin-3-yl)-2-(4-acetamidopyridin-2-yl)pyrazolo[5,1-b]thiazole-7-carboxamide

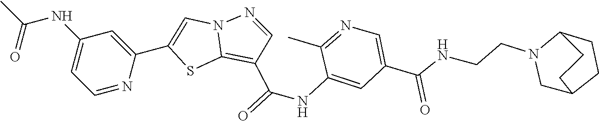 [SMILES: CC(=O)Nc1ccnc(-c2cn3ncc(C(=O)Nc4cc(C(=O)NCCN5CC6CCC5CC6)cnc4C)c3s2)c1]

### Step a: N-(2-(tributylstannyl)pyridin-4-yl)acetamide

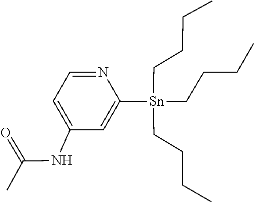 [SMILES: CCC[CH2][Sn]([CH2]CCC)([CH2]CCC)[c]1cc(NC(C)=O)ccn1]

To a solution of N-(2-bromopyridin-4-yl)acetamide (500 mg, 2.3 mmol), lithium chloride (500 mg, 12 mmol) and tricyclohexyl phosphine (130 mg, 0.46 mmol) in 1,4-dioxane (50 mL) was added hexabutylditin (6.0 g, 10 mmol). The resultant mixture was purged with N₂ for 5 minutes and then treated with tris(dibenzylideneacetone)dipalladium (210 mg, 0.23 mmol). The resultant mixture was purged with N₂ for another 5 minutes and then stirred while heating at 100° C. for 12 hours. The crude product was purified by column on silica gel (ethyl acetate/petroleum ether (0:100-100:0)) to give N-(2-(tributylstannyl)pyridin-4-yl)acetamide (470 mg, 23%) as light yellow solid. LCMS (ESI): mass calcd. for C₁₉H₃₄N₂OSn, 425.2; m/z found, 427.2 [M+H]⁺.

### Step b: N-(5-((2-(2-azabicyclo[2.2.2]octan-2-yl)ethyl)carbamoyl)-2-methylpyridin-3-yl)-2-(4-acetamidopyridin-2-yl)pyrazolo[5,1-b]thiazole-7-carboxamide

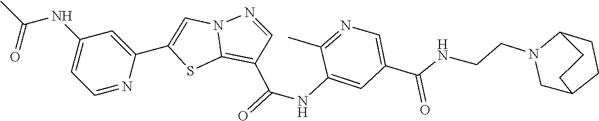 [SMILES: CC(=O)Nc1ccnc(-c2cn3ncc(C(=O)Nc4cc(C(=O)NCCN5CC6CCC5CC6)cnc4C)c3s2)c1]

To a mixture of N-(5-((2-(2-azabicyclo[2.2.2]octan-2-yl)ethyl)carbamoyl)-2-methylpyridin-3-yl)-2-bromopyrazolo[5,1-b]thiazole-7-carboxamide (200 mg, 0.39 mmol), N-(2-(tributylstannyl)pyridin-4-yl)acetamide (420 mg, 0.47 mmol) in dioxane (20 mL), CuI (20 mg, 0.11 mmol) were added a 40 mL under an atmosphere of nitrogen. Then palladium tetrakis(triphenylphosphine) (132 mg, 0.11 mmol) was added. N₂ was bubbled into the mixture for 5 min, and the vial was sealed with a rubber septum. The reaction vessel was gradually warm to 100° C. over the course of 10 min, after which time stirring was continued for 12 h. Then reaction mixture was concentrated under vacuum to give the crude product, which was purified by preparative high-performance liquid chromatography over column: Boston Green ODS 150*30 mm*5 um to give the title compound N-(5-((2-(2-azabicyclo[2.2.2]octan-2-yl)ethyl)carbamoyl)-2-methylpyridin-3-yl)-2-(4-acetamidopyridin-2-yl)pyrazolo[5,1-b]thiazole-7-carboxamide (21 mg, 8%) as a white solid. LCMS (ESI): mass calcd. for C₂₉H₃₂N₈O₃S. HCO2H, 618.7; m/z found, 573.3 [M+H]⁺. ¹H NMR (400 MHz, METHANOL-d₄) δ 8.81 (s, 1H), 8.71 (br d, J=8.9 Hz, 1H), 8.55 (br s, 1H), 8.49 (br d, J=3.3 Hz, 1H), 8.44-8.34 (m, 2H), 8.18 (br d, J=8.9 Hz, 1H), 7.49-7.41 (m, 1H), 3.77 (br t, J=6.0 Hz, 2H), 3.56 (br s, 1H), 3.44 (br t, J=6.0 Hz, 2H), 3.39 (br s, 2H), 2.65 (s, 3H), 2.25-2.12 (m, 5H), 2.00 (br s, 1H), 1.92-1.71 (m, 6H).

### Example 147: N-(5-((2-(2-azabicyclo[2.2.2]octan-2-yl)ethyl)carbamoyl)-2-methylpyridin-3-yl)-2-(5-acetamidopyridin-3-yl)pyrazolo[5,1-b]thiazole-7-carboxamide

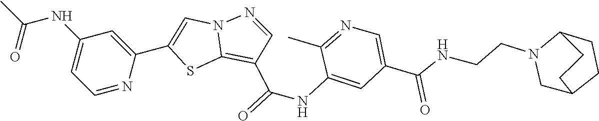 [SMILES: CC(=O)Nc1ccnc(-c2cn3ncc(C(=O)Nc4cc(C(=O)NCCN5CC6CCC5CC6)cnc4C)c3s2)c1]

To a solution of N-(5-((2-(2-azabicyclo[2.2.2]octan-2-yl)ethyl)carbamoyl)-2-methylpyridin-3-yl)-2-bromopyrazolo[5,1-b]thiazole-7-carboxamide (150 mg, 0.29 mmol), Cs₂CO₃ (283 mg, 0.87 mmol) and N-(5-(4,4,5,5-tetramethyl-1,3,2-dioxaborolan-2-yl)pyridin-3-yl)acetamide (91 mg, 0.35 mmol) in dioxane:H₂O=4:1 (20 mL) was added Pd(dppf)Cl₂·CH₂Cl₂ (71 mg, 0.087 mmol). The resulting mixture was stirred at 100° C. for 12 hours. The mixture was filtered through a celite pad and washed with dichloromethane (10 mL×3), then the filter was evaporated under vacuum to give residue, which was purified by column chromatography over silica gel (eluent: Petroleum ether/ethyl acetate=0:100 to 100:0 then ethyl acetate/methanol=90:10) to give the crude product. The crude product was purified by preparative high-performance liquid chromatography over column: Phenomenex Gemini-NX C18 75*30 mm*3 um to give title compound N-(5-((2-(2-azabicyclo[2.2.2]octan-2-yl)ethyl)carbamoyl)-2-methylpyridin-3-yl)-2-(5-acetamidopyridin-3-yl)pyrazolo[5,1-b]thiazole-7-carboxamide (23 mg, 13%) as a white solid. LCMS (ESI): mass calcd. for C₂₉H₃₂N₈O₃S, 572.7; m/z found, 573.3 [M+H]⁺. ¹H NMR (400 MHz, METHANOL-d₄) δ 8.83 (d, J=1.8 Hz, 1H), 8.72 (s, 1H), 8.68 (d, J=2.0 Hz, 1H), 8.66 (d, J=1.9 Hz, 1H), 8.55 (s, 1H), 8.52 (s, 2H), 8.41 (d, J=1.7 Hz, 1H), 3.76 (br t, J=6.0 Hz, 2H), 3.54 (br s, 1H), 3.41 (br t, J=5.8 Hz, 4H), 2.66 (s, 3H), 2.22 (s, 3H), 2.17 (br d, J=4.8 Hz, 2H), 2.00 (br s, 1H), 1.92-1.76 (m, 6H).

### Example 148: N-(5-((2-(2-azabicyclo[2.2.2]octan-2-yl)ethyl)carbamoyl)-2-methylpyridin-3-yl)-2-(pyridin-4-yl)pyrazolo[5,1-b]thiazole-7-carboxamide

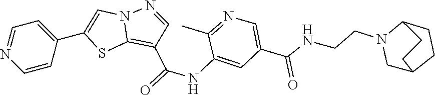 [SMILES: Cc1ncc(C(=O)NCCN2CC3CCC2CC3)cc1NC(=O)c1cnn2cc(-c3ccncc3)sc12]

To a solution of N-(5-((2-(2-azabicyclo[2.2.2]octan-2-yl)ethyl)carbamoyl)-2-methylpyridin-3-yl)-2-bromopyrazolo[5,1-b]thiazole-7-carboxamide (200 mg, 0.39 mmol), Cs₂CO₃ (378 mg, 1.2 mmol) and pyridin-4-ylboronic acid (62 mg, 0.5 mmol) in dioxane:H ₂O=4:1 (20 mL) was added Pd(dppf)Cl₂·CH₂Cl₂ (126 mg, 0.16 mmol). The resulting mixture was stirred at 100° C. for 12 hours. The mixture was filtered through a celite pad and washed with dichloromethane (10 mL×3), then the filter was evaporated under vacuum to give residue, which was purified by column chromatography over silica gel (eluent: Petroleum ether/ethyl acetate=0:100 to 100:0 then ethyl acetate/methanol=90:10) to give the crude product. The crude product was purified by preparative high-performance liquid chromatography over column: Boston Green ODS 150*30 mm*5 um to give title compound N-(5-((2-(2-azabicyclo[2.2.2]octan-2-yl)ethyl)carbamoyl)-2-methylpyridin-3-yl)-2-(pyridin-4-yl)pyrazolo[5,1-b]thiazole-7-carboxamide (43.5 mg, 18.8%) as a white solid. LCMS (ESI): mass calcd. for C₂₇H₂₉N₇O₂S, 515.6; m/z found, 516.3 [M+H]⁺. ¹H NMR (400 MHz, METHANOL-d₄) δ 8.88 (s, 1H), 8.79 (d, J=1.8 Hz, 1H), 8.61 (d, J=6.0 Hz, 2H), 8.50 (s, 1H), 8.36 (d, J=1.8 Hz, 1H), 7.73 (d, J=6.2 Hz, 2H), 3.73 (br t, J=6.0 Hz, 2H), 3.53 (br s, 1H), 3.40 (br t, J=6.1 Hz, 4H), 2.61 (s, 3H), 2.14 (br s, 2H), 1.96 (br s, 1H), 1.87-1.74 (m, 6H).

### Example 149: N-(5-((2-(2-azabicyclo[2.2.2]octan-2-yl)ethyl)carbamoyl)-2-methylpyridin-3-yl)-2-(pyridin-3-yl)pyrazolo[5,1-b]thiazole-7-carboxamide

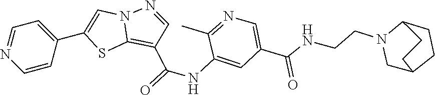 [SMILES: Cc1ncc(C(=O)NCCN2CC3CCC2CC3)cc1NC(=O)c1cnn2cc(-c3ccncc3)sc12]

To a solution of N-(5-((2-(2-azabicyclo[2.2.2]octan-2-yl)ethyl)carbamoyl)-2-methylpyridin-3-yl)-2-bromopyrazolo[5,1-b]thiazole-7-carboxamide (200 mg, 0.39 mmol), Cs₂CO₃ (378 mg, 1.2 mmol) and pyridin-3-ylboronic acid (62 mg, 0.5 mmol) in dioxane:H ₂O=4:1 (20 mL) was added Pd(dppf)Cl₂·CH₂Cl₂ (126 mg, 0.16 mmol). The resulting mixture was stirred at 100° C. for 12 hours. The mixture was filtered through a celite pad and washed with dichloromethane (10 mL×3), then the filter was evaporated under vacuum to give residue, which was purified by column chromatography over silica gel (eluent: Petroleum ether/ethyl acetate=0:100 to 100:0 then ethyl acetate/methanol=90:10) to give the crude product. The crude product was purified by preparative high-performance liquid chromatography over column: Boston Green ODS 150*30 mm*5 um to give title compound N-(5-((2-(2-azabicyclo[2.2.2]octan-2-yl)ethyl)carbamoyl)-2-methylpyridin-3-yl)-2-(pyridin-4-yl)pyrazolo[5,1-b]thiazole-7-carboxamide (54.4 mg, 24%) as a white solid. LCMS (ESI): mass calcd. for C₂₇H₂₉N₇O₂S, 515.6; m/z found, 516.3 [M+H]⁺. ¹H NMR (400 MHz, METHANOL-d₄) δ 8.91 (d, J=2.0 Hz, 1H), 8.81 (d, J=1.9 Hz, 1H), 8.71 (s, 1H), 8.59 (dd, J=1.4, 4.8 Hz, 1H), 8.53 (s, 1H), 8.49 (s, 1H), 8.38 (d, J=2.0 Hz, 1H), 8.16 (td, J=1.9, 8.2 Hz, 1H), 7.56 (dd, J=4.9, 8.0 Hz, 1H), 3.75 (t, J=6.1 Hz, 2H), 3.51 (br s, 1H), 3.40 (br t, J=6.1 Hz, 2H), 3.35 (br s, 2H), 2.63 (s, 3H), 2.22-2.09 (m, 2H), 1.97 (br d, J=2.5 Hz, 1H), 1.89-1.73 (m, 6H).

### Example 150: N-(5-((2-(2-azabicyclo[2.2.2]octan-2-yl)ethyl)carbamoyl)-2-methylpyridin-3-yl)-2-(1-(2-amino-2-oxoethyl)-1H-pyrazol-4-yl)pyrazolo[5,1-b]thiazole-7-carboxamide

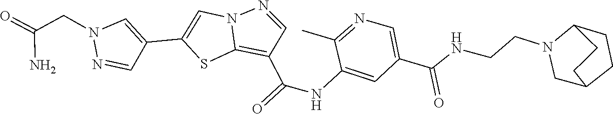 [SMILES: Cc1ncc(C(=O)NCCN2CC3CCC2CC3)cc1NC(=O)c1cnn2cc(-c3cnn(CC(N)=O)c3)sc12]

To a solution of N-(5-((2-(2-azabicyclo[2.2.2]octan-2-yl)ethyl)carbamoyl)-2-methylpyridin-3-yl)-2-bromopyrazolo[5,1-b]thiazole-7-carboxamide (50 mg, 0.097 mmol), Cs₂CO₃ (94 mg, 0.29 mmol) and 2-(4-(4,4,5,5-tetramethyl-1,3,2-dioxaborolan-2-yl)-1H-pyrazol-1-yl)acetonitrile (27 mg, 0.12 mmol) in dioxane:H₂O=4:1 (10 mL) was added Pd(dppf)Cl₂·CH₂Cl₂ (24 mg, 0.029 mmol). The resulting mixture was stirred at 100° C. for 12 hours. The mixture was filtered through a celite pad and washed with dichloromethane (10 mL×3), then the filter was evaporated under vacuum to give residue, which was purified by column chromatography over silica gel (eluent: Petroleum ether/ethyl acetate=0:100 to 100:0 then ethyl acetate/methanol=70:30) to give the crude product. The crude product was purified by preparative high-performance liquid chromatography over column: Phenomenex Gemini-NX C18 75*30 mm*3 um to give title compound N-(5-((2-(2-azabicyclo[2.2.2]octan-2-yl)ethyl)carbamoyl)-2-methylpyridin-3-yl)-2-(1-(cyanomethyl)-1H-pyrazol-4-yl)pyrazolo[5,1-b]thiazole-7-carboxamide (5.8 mg, 10%) as a white solid. LCMS (ESI): mass calcd. for C₂₇H₃₁N₉O₃S, 561.7; m/z found, 562.3 [M+H]⁺. ¹H NMR (400 MHz, METHANOL-d₄) δ 8.78 (d, J=2.0 Hz, 1H), 8.50 (br s, 1H), 8.40 (s, 1H), 8.35 (d, J=1.8 Hz, 1H), 8.26 (s, 1H), 8.09 (s, 1H), 7.87 (s, 1H), 4.92 (s, 2H), 3.73 (br t, J=6.0 Hz, 2H), 3.53 (br s, 1H), 3.40 (br t, J=6.1 Hz, 2H), 3.33 (br s, 2H), 2.61 (s, 3H), 2.14 (br s, 3H), 1.96 (br s, 1H), 1.89-1.70 (m, 5H).

### Example 151: N-(5-((2-(2-azabicyclo[2.2.2]octan-2-yl)ethyl)carbamoyl)-2-methylpyridin-3-yl)-2-(1-(cyanomethyl)-1H-pyrazol-4-yl)pyrazolo[5,1-b]thiazole-7-carboxamide

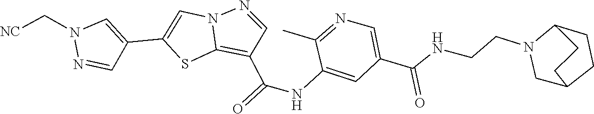 [SMILES: Cc1ncc(C(=O)NCCN2CC3CCC2CC3)cc1NC(=O)c1cnn2cc(-c3cnn(CC#N)c3)sc12]

To a solution of N-(5-((2-(2-azabicyclo[2.2.2]octan-2-yl)ethyl)carbamoyl)-2-methylpyridin-3-yl)-2-bromopyrazolo[5,1-b]thiazole-7-carboxamide (300 mg, 0.58 mmol), Cs₂CO₃ (567 mg, 1.7 mmol) and 2-(4-(4,4,5,5-tetramethyl-1,3,2-dioxaborolan-2-yl)-1H-pyrazol-1-yl)acetonitrile (176 mg, 0.75 mmol) in dioxane:H₂O=4:1 (18 mL) was added Pd(dppf)Cl₂·CH₂Cl₂ (142 mg, 0.17 mmol). The resulting mixture was stirred at 70° C. for 12 hours. The mixture was filtered through a celite pad and washed with dichloromethane (10 mL×3), then the filter was evaporated under vacuum to give residue, which was purified by column chromatography over silica gel (eluent: Petroleum ether/ethyl acetate=0:100 to 100:0 then ethyl acetate/methanol=80:20) to give the crude product. The crude product was purified by preparative high-performance liquid chromatography over column: Phenomenex Gemini-NX C18 75*30 mm*3 um to give the crude product and the crude product was purified by Supercritical Fluid Chromatography over column: DAICEL CHIRALCEL OJ 250 mm*30 mm, 10 um to give title compound N-(5-((2-(2-azabicyclo[2.2.2]octan-2-yl)ethyl)carbamoyl)-2-methylpyridin-3-yl)-2-(1-(cyanomethyl)-1H-pyrazol-4-yl)pyrazolo[5,1-b]thiazole-7-carboxamide (35.7 mg, 10%) as a white solid. LCMS (ESI): mass calcd. for C₂₇H₃₁N₉O₂S, 543.6; m/z found, 544.3 [M+H]⁺. ¹H NMR (400 MHz, METHANOL-d₄) δ 8.76 (d, J=1.8 Hz, 1H), 8.40 (s, 1H), 8.31-8.27 (m, 2H), 8.17 (s, 1H), 7.95 (s, 1H), 5.37 (s, 2H), 3.54 (t, J=6.9 Hz, 2H), 2.90-2.80 (m, 4H), 2.73 (br s, 1H), 2.59 (s, 3H), 2.00 (br d, J=10.6 Hz, 2H), 1.71-1.54 (m, 7H).

### Example 152: N-(5-((2-(3,3-difluoroazetidin-1-yl)ethyl)carbamoyl)-2-methylpyridin-3-yl)-2-(pyridin-4-yl)pyrazolo[5,1-b]thiazole-7-carboxamide

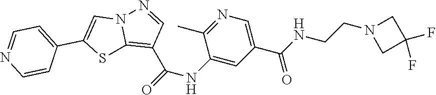 [SMILES: Cc1ncc(C(=O)NCCN2CC(F)(F)C2)cc1NC(=O)c1cnn2cc(-c3ccncc3)sc12]

### Step a: methyl 6-methyl-5-(2-(pyridin-4-yl)pyrazolo[5,1-b]thiazole-7-carboxamido)nicotinate

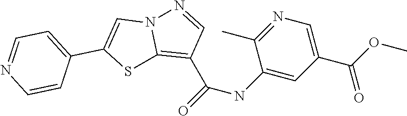 [SMILES: COC(=O)c1cnc(C)c(NC(=O)c2cnn3cc(-c4ccncc4)sc23)c1]

To a solution of methyl 5-(2-bromopyrazolo[5,1-b]thiazole-7-carboxamido)-4-methylthiophene-2-carboxylate (500 mg, 1.2 mmol) and pyridin-4-ylboronic acid (170 mg, 1.4 mmol) in 1,4-dioxane (20 mL) and H₂O (5 mL) was added Pd(dppf)Cl₂·CH₂Cl₂ (188 mg, 0.23 mmol) and K₂CO₃ (478 mg, 3.5 mmol) under N₂. The resulting mixture was stirred at 95° C. for 16 h before cooled to 25° C. Then the reaction mixture was concentrated under reduced pressure to afford the crude product methyl 6-methyl-5-(2-(pyridin-4-yl)pyrazolo[5,1-b]thiazole-7-carboxamido)nicotinate (1 g, 27%) as a brown solid. LCMS (ESI): mass calcd. for C₂₀H₁₇N₅O₃S, 407.4; m/z found, 408.0 [M+H]⁺.

### Step b: 6-methyl-5-(2-(pyridin-4-yl)pyrazolo[5,1-b]thiazole-7-carboxamido)nicotinic acid

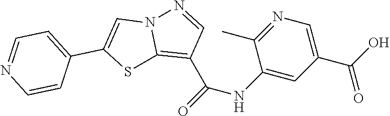 [SMILES: Cc1ncc(C(=O)O)cc1NC(=O)c1cnn2cc(-c3ccncc3)sc12]

To a solution of methyl 6-methyl-5-(2-(pyridin-4-yl)pyrazolo[5,1-b]thiazole-7-carboxamido)nicotinate (1 g, 0.31 mmol) in THF (30 mL) was added lithium hydroxide hydrate (13 mg, 0.31 mmol) in H₂O (10 mL) at room temperature. The reaction mixture was stirred at 25° C. for 2 h. The mixture was adjusted to pH=3-4 with HCl (aq, 2 M). The mixture was filtered and washed with water (10 mL×3). The solid was evaporated under vacuum to give desired product 6-methyl-5-(2-(pyridin-4-yl)pyrazolo[5,1-b]thiazole-7-carboxamido)nicotinic acid (90 mg, 66%) as brown solid. LCMS (ESI): mass calcd. for C₁₈H₁₃N₅O₃S, 379.4; m/z found, 380.0 [M+H]⁺.

### Step c: N-(5-((2-(3,3-difluoroazetidin-1-yl)ethyl)carbamoyl)-2-methylpyridin-3-yl)-2-(pyridin-4-yl)pyrazolo[5,1-b]thiazole-7-carboxamide

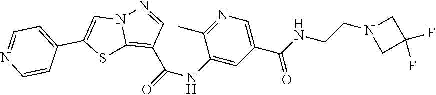 [SMILES: Cc1ncc(C(=O)NCCN2CC(F)(F)C2)cc1NC(=O)c1cnn2cc(-c3ccncc3)sc12]

To a solution of 6-methyl-5-(2-(pyridin-4-yl)pyrazolo[5,1-b]thiazole-7-carboxamido)nicotinic acid (80 mg, 0.18 mmol), 2-(3,3-difluoroazetidin-1-yl)ethanamine (25 mg, 0.18 mmol) and N-ethyl-N-isopropylpropan-2-amine (0.12 mL, 0.74 mmol) in DMF (3 mL) was added HATU (91 mg, 0.24 mmol). The resulting mixture was stirred at 25° C. for 2 hours and then concentrated under vacuum to give the crude product, which was purified by preparative high-performance liquid chromatography over column: Welch Xtimate C18 150*30 mm*5 um to give the title compound N-(5-((2-(3,3-difluoroazetidin-1-yl)ethyl)carbamoyl)-2-methylpyridin-3-yl)-2-(pyridin-4-yl)pyrazolo[5,1-b]thiazole-7-carboxamide (40 mg, 44%) as a yellow solid. LCMS (ESI): mass calcd. for C₂₃H₂₁F₂N₇O₂S, 497.5; m/z found, 498.0 [M+H]⁺. ¹HNMR (400 MHz, CHLOROFORM-d) δ 8.70 (d, J=1.98 Hz, 1H) 8.67 (d, J=5.95 Hz, 2H) 8.61 (s, 1H) 8.20 (s, 1H) 8.09 (s, 1H) 7.40 (d, J=5.95 Hz, 2H) 7.37 (s, 1H) 6.60 (br s, 1H) 3.60 (t, J=12.02 Hz, 4H) 3.42 (br d, J=5.51 Hz, 2H) 2.77 (br d, J=5.73 Hz, 2H) 2.61 (s, 3H).

### Example 153: N-(5-((2-(5-azaspiro[2.4]heptan-5-yl)ethyl)carbamoyl)-2-methylpyridin-3-yl)-2-(pyridin-4-yl)pyrazolo[5,1-b]thiazole-7-carboxamide

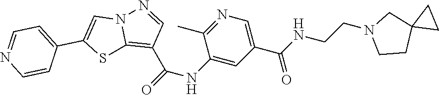 [SMILES: Cc1ncc(C(=O)NCCN2CCC3(CC3)C2)cc1NC(=O)c1cnn2cc(-c3ccncc3)sc12]

To a solution of 6-methyl-5-(2-(pyridin-4-yl)pyrazolo[5,1-b]thiazole-7-carboxamido)nicotinic acid (80 mg, 0.20 mmol), 2-(5-azaspiro[2.4]heptan-5-yl)ethanamine (28 mg, 0.20 mmol) and N-ethyl-N-isopropylpropan-2-amine (0.13 mL, 0.79 mmol) in DMF (3 mL) was added HATU (98 mg, 0.26 mmol). The resulting mixture was stirred at 25° C. for 2 hours and then concentrated under vacuum to give the crude product, which was purified by preparative high-performance liquid chromatography over column: Welch Xtimate C18 150*30 mm*5 um to give the title compound: N-(5-((2-(5-azaspiro[2.4]heptan-5-yl)ethyl)carbamoyl)-2-methylpyridin-3-yl)-2-(pyridin-4-yl)pyrazolo[5,1-b]thiazole-7-carboxamide (16 mg, 16%) as a white solid. LCMS (ESI): mass calcd. for C₂₆H₂₇N₇O₂S, 501.6; m/z found, 502.1 [M+H]⁺. ¹H NMR (400 MHz, CHLOROFORM-d) δ 8.65-8.80 (m, 3H) 8.57 (br s, 1H) 8.25 (s, 1H) 8.18 (s, 1H) 7.61 (br s, 1H) 7.44 (d, J=5.95 Hz, 2H) 7.01 (br s, 1H) 3.49-3.61 (m, 2H) 2.69-2.81 (m, 4H) 2.64 (s, 3H) 2.55 (s, 2H) 1.82 (br t, J=6.84 Hz, 2H) 0.56 (br d, J=11.47 Hz, 4H).

### Example 154: N-(5-((2-(4,4-difluoropiperidin-1-yl)ethyl)carbamoyl)-2-methylpyridin-3-yl)-2-(pyridin-4-yl)pyrazolo[5,1-b]thiazole-7-carboxamide

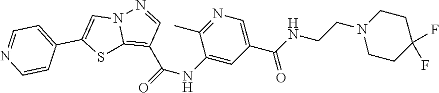 [SMILES: Cc1ncc(C(=O)NCCN2CCC(F)(F)CC2)cc1NC(=O)c1cnn2cc(-c3ccncc3)sc12]

To a solution of 6-methyl-5-(2-(pyridin-4-yl)pyrazolo[5,1-b]thiazole-7-carboxamido)nicotinic acid (178 mg, 0.19 mmol), HATU (93 mg, 0.25 mmol) and N,N-diisopropylethylamine (0.13 mL, 0.76 mmol) in DMF (5 mL) was added 2-(4,4-difluoropiperidin-1-yl)ethanamine (37 mg, 0.23 mmol). The resulting mixture was stirred at room temperature for 2 h and then concentrated under vacuum to give the crude product, which was purified by preparative high-performance liquid chromatography over column: Phenomenex Genimi NX C18 150*40 mm*5 um to give the title compound N-(5-((2-(4,4-difluoropiperidin-1-yl)ethyl)carbamoyl)-2-methylpyridin-3-yl)-2-(pyridin-4-yl)pyrazolo[5,1-b]thiazole-7-carboxamide (62.2 mg, 61%) as a white solid. LCMS (ESI): mass calcd. for C₂₅H₂₅F₂N₇O₂S, 525.5; m/z found, 526.2 [M+H]⁺. ¹H NMR (400 MHz, DMSO-d₆) δ 10.06 (s, 1H) 9.29 (s, 1H) 8.77 (d, J=1.54 Hz, 1H) 8.58-8.70 (m, 4H) 8.20 (s, 1H) 8.10 (s, 1H) 7.73 (d, J=4.94 Hz, 2H) 3.42 (br d, J=1.10 Hz, 2H) 2.49-2.66 (m, 9H) 1.85-2.13 (m, 4H).

### Example 155: N-(5-((2-(3,3-dimethylazetidin-1-yl)ethyl)carbamoyl)-2-methylpyridin-3-yl)-2-(pyridin-4-yl)pyrazolo[5,1-b]thiazole-7-carboxamide

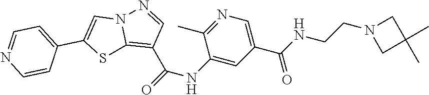 [SMILES: Cc1ncc(C(=O)NCCN2CC(C)(C)C2)cc1NC(=O)c1cnn2cc(-c3ccncc3)sc12]

To a solution of 6-methyl-5-(2-(pyridin-4-yl)pyrazolo[5,1-b]thiazole-7-carboxamido)nicotinic acid (300 mg, 0.57 mmol), HATU (388 mg, 1.0 mmol) and N,N-diisopropylethylamine (0.30 mL, 1.7 mmol) in N,N-dimethylformamide (8 mL) was added 2-(3,3-dimethylazetidin-1-yl)ethanamine (145 mg, 1.1 mmol). The mixture stirred at room temperature for 1 hour and then concentrated under vacuum to give the crude product, which was purified by preparative high-performance liquid chromatography over column: Phenomenex Gemini-NX C18 75*30 mm*3 um to give the title compound N-(5-((2-(3,3-dimethylazetidin-1-yl)ethyl)carbamoyl)-2-methylpyridin-3-yl)-2-(pyridin-4-yl)pyrazolo[5,1-b]thiazole-7-carboxamide (30 mg, 10%) as a white solid. LCMS (ESI): mass calcd. for C₂₅H₂₇N₇O₂S, 489.2; m/z found, 490.1 [M+H]⁺. ¹H NMR (400 MHz, METHANOL-d₄) δ 8.92 (s, 1H), 8.81 (d, J=2.0 Hz, 1H), 8.68-8.63 (m, 2H), 8.53 (s, 1H), 8.34 (d, J=2.0 Hz, 1H), 7.83-7.75 (m, 2H), 3.44 (t, J=6.6 Hz, 2H), 3.17 (s, 4H), 2.78 (t, J=6.6 Hz, 2H), 2.63 (s, 3H), 1.26 (s, 6H).

### Example 156: N-(5-((2-(3,3-dimethylpyrrolidin-1-yl)ethyl)carbamoyl)-2-methylpyridin-3-yl)-2-(pyridin-4-yl)pyrazolo[5,1-b]thiazole-7-carboxamide

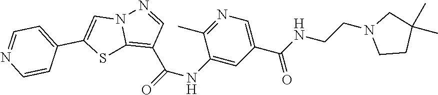 [SMILES: Cc1ncc(C(=O)NCCN2CCC(C)(C)C2)cc1NC(=O)c1cnn2cc(-c3ccncc3)sc12]

### Step a: 2-(3,3-dimethylpyrrolidin-1-yl)acetonitrile

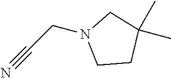

A mixture of 3,3-dimethylpyrrolidine (450 mg, 3.3 mmol) and K₂CO₃ (1.4 g, 10.0 mmol) in ACN (10 mL), then 2-bromoacetonitrile (0.31 mL, 5.0 mmol) was added. The reaction mixture was stirred at 50° C. for 16 hours. The resulting mixture was quenched with water (20 mL) and extracted with ethyl acetate (50 mL×3). The combined organic phases were dried over anhydrous Na₂SO₄ and filtered. The filtrate was concentrated under reduced pressure to give crude product, which was purified by column chromatography over silica gel (eluent: petroleum ether:ethyl acetate from 100:0 to 65:35) to give the title compound 2-(3,3-dimethylpyrrolidin-1-yl)acetonitrile (350 mg, 76%) as a colorless liquid. LCMS (ESI): mass calcd. for C₈H₁₄N₂, 138.21; m/z found, 139.300 [M+H]⁺. ¹H NMR (400 MHz, CHLOROFORM-d) δ 3.68 (s, 2H), 2.85 (br t, J=7.0 Hz, 2H), 2.54 (s, 2H), 1.70 (t, J=7.0 Hz, 2H), 1.15 (s, 6H).

### Step b: 2-(cyclobutyl(2-methoxyethyl)amino)acetonitrile

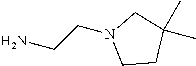

To a solution of 2-(3,3-dimethylpyrrolidin-1-yl)acetonitrile (350 mg, 2.5 mmol) in THE (8 mL) was added lithium aluminium hydride (144 mg, 3.8 mmol) by portions at 0° C. (ice/water). The resultant mixture was stirred at room temperature for 1.5 h, then the reaction mixture was quenched with water (0.5 mL) at 0° C. The reaction mixture was filtered. And the filtrate was concentrated to dryness under reduced pressure to afford the crude product 2-(cyclobutyl(2-methoxyethyl)amino)acetonitrile (250 mg, 69%) as a colorless oil. LCMS (ESI): mass calcd. for C₈H₁₈N₂, 142.242; m/z found, 143.200 [M+H]⁺. ¹H NMR (400 MHz, CHLOROFORM-d) δ 2.76-2.66 (m, 2H), 2.57 (br t, J=7.1 Hz, 2H), 2.45 (t, J=6.3 Hz, 2H), 2.28 (s, 2H), 2.21-2.06 (m, 2H), 1.60-1.46 (m, 2H), 1.04-0.98 (m, 6H).

### Step c: N-(5-((2-(3,3-dimethylpyrrolidin-1-yl)ethyl)carbamoyl)-2-methylpyridin-3-yl)-2-(pyridin-4-yl)pyrazolo[5,1-b]thiazole-7-carboxamide

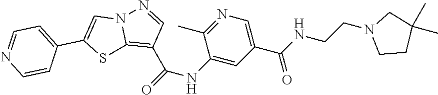 [SMILES: Cc1ncc(C(=O)NCCN2CCC(C)(C)C2)cc1NC(=O)c1cnn2cc(-c3ccncc3)sc12]

To a solution of 6-methyl-5-(2-(1-methyl-1H-pyrazol-4-yl)pyrazolo[5,1-b]thiazole-7-carboxamido)nicotinic acid (100 mg, 0.26 mmol), HATU (150 mg, 0.40 mmol) and N,N-diisopropylethylamine (0.17 mL, 1.1 mmol) in DMF (2 mL) was added 2-(cyclobutyl(2-methoxyethyl)amino)acetonitrile (49 mg, 0.34 mmol). The resulting mixture was stirred at room temperature for 2 h and then concentrated under vacuum to give the crude product, which was purified by preparative high-performance liquid chromatography over column: Welch Xtimate C18 150*30 mm*5 um and column: DAICEL CHIRALCEL OD-H (250 mm*30 mm, 5 um) to give the title compound N-(5-((2-(3,3-dimethylpyrrolidin-1-yl)ethyl)carbamoyl)-2-methylpyridin-3-yl)-2-(pyridin-4-yl)pyrazolo[5,1-b]thiazole-7-carboxamide (20 mg, 65%) as a white solid. LCMS (ESI): mass calcd. for C₂₆H₂₉N₇O₂S, 503.619; m/z found, 504.40 [M+H]⁺. ¹H NMR (400 MHz, METHANOL-d₄) δ 8.91 (s, 1H), 8.81 (d, J=1.5 Hz, 1H), 8.65 (br d, J=5.5 Hz, 2H), 8.52 (s, 1H), 8.34 (s, 1H), 7.77 (br d, J=6.0 Hz, 2H), 3.58 (t, J=6.9 Hz, 2H), 2.79 (td, J=6.7, 13.4 Hz, 4H), 2.63 (s, 3H), 2.54 (s, 2H), 1.68 (t, J=6.9 Hz, 2H), 1.14 (s, 6H).

### Example 157: N-(5-((2-(4,4-difluoropiperidin-1-yl)ethyl)carbamoyl)-2-methylpyridin-3-yl)-2-(pyridin-3-yl)pyrazolo[5,1-b]thiazole-7-carboxamide

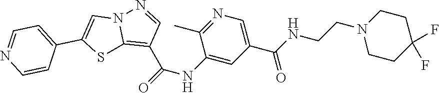 [SMILES: Cc1ncc(C(=O)NCCN2CCC(F)(F)CC2)cc1NC(=O)c1cnn2cc(-c3ccncc3)sc12]

### Step a: 6-methyl-5-(2-(pyridin-3-yl)pyrazolo[5,1-b]thiazole-7-carboxamido)nicotinic acid

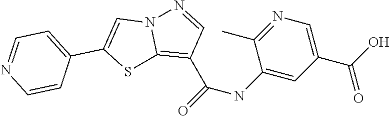 [SMILES: Cc1ncc(C(=O)O)cc1NC(=O)c1cnn2cc(-c3ccncc3)sc12]

To a mixture of ethyl 5-(2-bromopyrazolo[5,1-b]thiazole-7-carboxamido)-6-methylnicotinate (210 mg, 0.49 mmol), pyridin-3-ylboronic acid (72 mg, 0.59 mmol) and Pd(dppf)Cl₂CH₂Cl₂ (100 mg, 0.12 mmol) in mixture of THF/H₂O (4:1, 15 mL) was added K₂CO₃ (203 mg, 1.5 mmol). The mixture was purged with N2 with 2 minutes. Then the reaction mixture was stirred at 90° C. for 30 hours. After cooling down room temperature, the mixture was filtered off and the filtrate was evaporated to give crude product, which was triturated with EtOAc (10 mL). The mixture was filtered. The filter cake was dried in vacuo to give the title compound 6-methyl-5-(2-(pyridin-3-yl)pyrazolo[5,1-b]thiazole-7-carboxamido)nicotinic acid (167 mg, 89%) as a brown solid. LCMS (ESI): mass calcd. for C₁₈H₁₃N₅O₃S, 379.1; m/z found, 380.2 [M+H]⁺.

### Step b: N-(5-((2-(4,4-difluoropiperidin-1-yl)ethyl)carbamoyl)-2-methylpyridin-3-yl)-2-(pyridin-3-yl)pyrazolo[5,1-b]thiazole-7-carboxamide

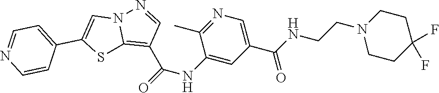 [SMILES: Cc1ncc(C(=O)NCCN2CCC(F)(F)CC2)cc1NC(=O)c1cnn2cc(-c3ccncc3)sc12]

To a solution of 6-methyl-5-(2-(pyridin-3-yl)pyrazolo[5,1-b]thiazole-7-carboxamido)nicotinic acid (140 mg, 0.37 mmol), HATU (253 mg, 0.67 mmol) and N,N-diisopropylethylamine (0.26 mL, 1.5 mmol) in N,N-dimethylformamide (8 mL) was added 2-(4,4-difluoropiperidin-1-yl)ethanamine (121 mg, 0.74 mmol). The mixture stirred at room temperature for 1 hour and then concentrated under vacuum to give the crude product, which was purified by preparative high-performance liquid chromatography over column: Phenomenex Gemini-NX C18 75*30 mm*3 um to give the title compound N-(5-((2-(4,4-difluoropiperidin-1-yl)ethyl)carbamoyl)-2-methylpyridin-3-yl)-2-(pyridin-3-yl)pyrazolo[5,1-b]thiazole-7-carboxamide (30 mg, 15%) as an off white solid. LCMS (ESI): mass calcd. for C₂₅H₂₅F₂N₇O₂S, 525.2; m/z found, 526.5 [M+H]⁺. ¹H NMR (400 MHz, METHANOL-d₄) δ 8.95 (d, J=2.1 Hz, 1H), 8.80 (d, J=2.0 Hz, 1H), 8.73 (s, 1H), 8.61 (dd, J=1.4, 4.8 Hz, 1H), 8.50 (s, 1H), 8.34 (d, J=2.0 Hz, 1H), 8.18 (br d, J=8.1 Hz, 1H), 7.59 (dd, J=4.9, 8.0 Hz, 1H), 3.58 (t, J=6.7 Hz, 2H), 2.73-2.66 (m, 6H), 2.64 (s, 3H), 2.03 (ddd, J=5.6, 13.4, 19.5 Hz, 4H).

### Example 158: N-(5-((2-(3,3-dimethylazetidin-1-yl)ethyl)carbamoyl)-2-methylpyridin-3-yl)-2-(pyridin-3-yl)pyrazolo[5,1-b]thiazole-7-carboxamide

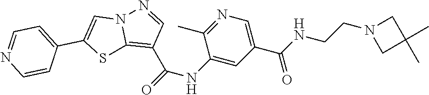 [SMILES: Cc1ncc(C(=O)NCCN2CC(C)(C)C2)cc1NC(=O)c1cnn2cc(-c3ccncc3)sc12]

To a solution of 6-methyl-5-(2-(pyridin-3-yl)pyrazolo[5,1-b]thiazole-7-carboxamido)nicotinic acid (160 mg, 0.42 mmol), HATU (289 mg, 0.76 mmol) and N,N-diisopropylethylamine (0.29 mL, 1.7 mmol) in N,N-dimethylformamide (8 mL) was added 2-(3,3-dimethylazetidin-1-yl)ethanamine (70 mg, 0.55 mmol). The mixture stirred at room temperature for 1 hour and then concentrated under vacuum to give the crude product, which was purified by preparative high-performance liquid chromatography over column: Phenomenex Gemini-NX C18 75*30 mm*3 um to give the title compound N-(5-((2-(3,3-dimethylazetidin-1-yl)ethyl)carbamoyl)-2-methylpyridin-3-yl)-2-(pyridin-3-yl)pyrazolo[5,1-b]thiazole-7-carboxamide (20 mg, 10%) as a gray solid. LCMS (ESI): mass calcd. for C₂₅H₂₇N₇O₂S, 489.2; m/z found, 490.3 [M+H]⁺. ¹H NMR (400 MHz, METHANOL-d₄) δ 8.95 (d, J=1.8 Hz, 1H), 8.81 (d, J=2.0 Hz, 1H), 8.73 (s, 1H), 8.61 (dd, J=1.4, 4.9 Hz, 1H), 8.51 (s, 1H), 8.34 (d, J=2.0 Hz, 1H), 8.21-8.16 (m, 1H), 7.59 (dd, J=4.9, 7.9 Hz, 1H), 3.44 (t, J=6.6 Hz, 2H), 3.16 (s, 4H), 2.77 (t, J=6.6 Hz, 2H), 2.64 (s, 3H), 1.26 (s, 6H).

### Example 159: N-(5-((2-(5-azaspiro[2.4]heptan-5-yl)ethyl)carbamoyl)-2-methylpyridin-3-yl)-2-(pyridin-3-yl)pyrazolo[5,1-b]thiazole-7-carboxamide

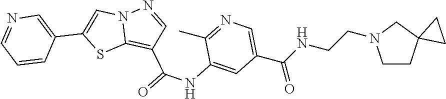 [SMILES: Cc1ncc(C(=O)NCCN2CCC3(CC3)C2)cc1NC(=O)c1cnn2cc(-c3cccnc3)sc12]

To a solution of 6-methyl-5-(2-(pyridin-3-yl)pyrazolo[5,1-b]thiazole-7-carboxamido)nicotinic acid (200 mg, 0.53 mmol), HATU (361 mg, 0.95 mmol) and N,N-diisopropylethylamine (0.28 mL, 1.6 mmol) in N,N-dimethylformamide (8 mL) was added 2-(5-azaspiro[2.4]heptan-5-yl)ethanamine (89 mg, 0.63 mmol). The mixture stirred at room temperature for 12 hours and then concentrated under vacuum to give the crude product, which was purified by preparative high-performance liquid chromatography over column: Phenomenex Gemini-NX C18 75*30 mm*3 um to give the title compound N-(5-((2-(5-azaspiro[2.4]heptan-5-yl)ethyl)carbamoyl)-2-methylpyridin-3-yl)-2-(pyridin-3-yl)pyrazolo[5,1-b]thiazole-7-carboxamide (Formic acid salt, 21 mg, 10%) as a white solid. LCMS (ESI): mass calcd. for C₂₆H₂₇N₇O₂S, 501.2; m/z found, 502.5 [M+H]⁺. ¹H NMR (400 MHz, METHANOL-d₄) δ 8.94 (d, J=2.0 Hz, 1H), 8.83 (d, J=2.0 Hz, 1H), 8.74 (s, 1H), 8.61 (dd, J=1.4, 4.8 Hz, 1H), 8.52 (s, 2H), 8.39 (d, J=2.0 Hz, 1H), 8.22-8.16 (m, 1H), 7.59 (dd, J=4.9, 7.7 Hz, 1H), 3.73 (t, J=6.1 Hz, 2H), 3.42 (br s, 2H), 3.27 (br d, J=5.5 Hz, 2H), 3.18 (br s, 2H), 2.65 (s, 3H), 2.08-2.02 (m, 2H), 0.81-0.71 (m, 4H).

### Example 160: N-(5-((2-(5-azaspiro[2.4]heptan-5-yl)ethyl)carbamoyl)-2-methylpyridin-3-yl)-2-(pyridin-2-yl)pyrazolo[5,1-b]thiazole-7-carboxamide

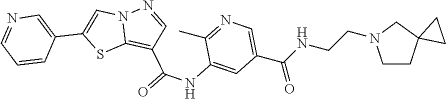 [SMILES: Cc1ncc(C(=O)NCCN2CCC3(CC3)C2)cc1NC(=O)c1cnn2cc(-c3cccnc3)sc12]

### Step a: ethyl 6-methyl-5-(2-(pyridin-2-yl)pyrazolo[5,1-b]thiazole-7-carboxamido)nicotinate

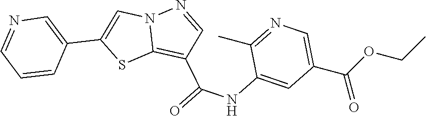 [SMILES: CCOC(=O)c1cnc(C)c(NC(=O)c2cnn3cc(-c4cccnc4)sc23)c1]

To a mixture of ethyl 5-(2-bromopyrazolo[5,1-b]thiazole-7-carboxamido)-6-methylnicotinate (200 mg, 0.49 mmol), 2-(tributylstannyl)pyridine (270 mg, 0.73 mmol) in DMF (15 mL) was added Pd(PPh₃)₄ (113 mg, 0.10 mmol). The mixture was purged with N₂ with 2 minutes. Then the reaction mixture was stirred at 100° C. for 15 hours. After cooling down room temperature, the mixture was filtered off and the filtrate was evaporated to give crude product, which was purified by column chromatography over silica gel (eluent: ethyl acetate) to give the title compound ethyl 6-methyl-5-(2-(pyridin-2-yl)pyrazolo[5,1-b]thiazole-7-carboxamido)nicotinate (100 mg, 38%) as a pale brown solid. LCMS (ESI): mass calcd. for C₂₀H₁₇N₅O₃S, 407.1; m/z found, 408.1 [M+H]⁺.

### Step b: 6-methyl-5-(2-(pyridin-2-yl)pyrazolo[5,1-b]thiazole-7-carboxamido)nicotinic acid

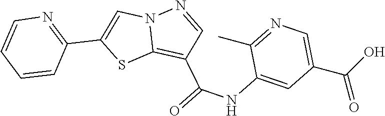 [SMILES: Cc1ncc(C(=O)O)cc1NC(=O)c1cnn2cc(-c3ccccn3)sc12]

To a solution of ethyl 6-methyl-5-(2-(pyridin-2-yl)pyrazolo[5,1-b]thiazole-7-carboxamido)nicotinate (220 mg, 0.54 mmol) in in mixture of THF/MeOH (1:1, 4 mL) was added 1M LiOH (0.81 mL, 0.81 mmol). The reaction was stirred at room temperature for 12 hours. The reaction mixture was concentrated in vacuo to give crude. The crude was dissolved in H₂O (10 mL). The mixture was adjusted to pH-5 with 1N HCl. The mixture was filtered. The filter cake was washed with H₂O (5 mL). The filter cake was triturated with EtOAc (10 mL). The mixture was filtered. The filter cake was dried in vacuo to give 6-methyl-5-(2-(pyridin-2-yl)pyrazolo[5,1-b]thiazole-7-carboxamido)nicotinic acid (180 mg, 88%) as a pale brown solid. LCMS (ESI): mass calcd. for C₂₀H₁₇N₅O₃S, 379.1; m/z found, 380.2 [M+H]+

### Step c: N-(5-((2-(5-azaspiro[2.4]heptan-5-yl)ethyl)carbamoyl)-2-methylpyridin-3-yl)-2-(pyridin-2-yl)pyrazolo[5,1-b]thiazole-7-carboxamide

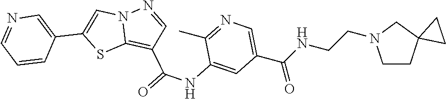 [SMILES: Cc1ncc(C(=O)NCCN2CCC3(CC3)C2)cc1NC(=O)c1cnn2cc(-c3cccnc3)sc12]

To a solution of 6-methyl-5-(2-(pyridin-2-yl)pyrazolo[5,1-b]thiazole-7-carboxamido)nicotinic acid (100 mg, 0.26 mmol), HATU (180 mg, 0.47 mmol) and N,N-diisopropylethylamine (0.14 mL, 0.79 mmol) in N,N-dimethylformamide (5 mL) was added 2-(5-azaspiro[2.4]heptan-5-yl)ethanamine (55 mg, 0.40 mmol). The mixture stirred at room temperature for 1 hour and then concentrated under vacuum to give the crude product, which was purified by preparative high-performance liquid chromatography over column: Phenomenex Gemini-NX C18 75*30 mm*3 um to give the title compound N-(5-((2-(5-azaspiro[2.4]heptan-5-yl)ethyl)carbamoyl)-2-methylpyridin-3-yl)-2-(pyridin-2-yl)pyrazolo[5,1-b]thiazole-7-carboxamide (35 mg, 26%) as a white solid. LCMS (ESI): mass calcd. for C₂₆H₂₇N₇O₂S, 501.2; m/z found, 502.4 [M+H]⁺. ¹H NMR (400 MHz, METHANOL-d₄) δ 8.86-8.80 (m, 2H), 8.60 (d, J=4.4 Hz, 1H), 8.49 (s, 1H), 8.36 (d, J=1.9 Hz, 1H), 8.01-7.96 (m, 1H), 7.95-7.87 (m, 1H), 7.42-7.35 (m, 1H), 3.61 (t, J=6.7 Hz, 2H), 2.93 (br t, J=7.0 Hz, 2H), 2.83 (br t, J=6.7 Hz, 2H), 2.73-2.68 (m, 2H), 2.64 (s, 3H), 1.90 (t, J=7.0 Hz, 2H), 0.68-0.57 (m, 4H).

### Example 161: N-(5-((2-(5-azaspiro[2.4]heptan-5-yl)ethyl)carbamoyl)-2-methylpyridin-3-yl)-2-(1-(2-methoxyethyl)-1H-pyrazol-4-yl)pyrazolo[5,1-b]thiazole-7-carboxamide

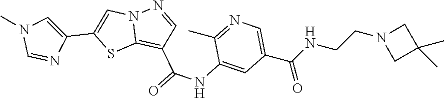

### Step a: ethyl 6-methyl-5-(2-(1-methyl-1H-imidazol-4-yl)pyrazolo[5,1-b]thiazole-7-carboxamido)nicotinate

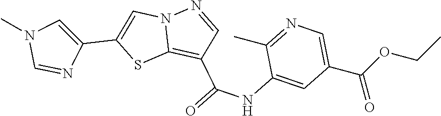 [SMILES: CCOC(=O)c1cnc(C)c(NC(=O)c2cnn3cc(-c4cn(C)cn4)sc23)c1]

To a solution of ethyl 5-(2-bromopyrazolo[5,1-b]thiazole-7-carboxamido)-6-methylnicotinate (250 mg, 0.62 mmol), 4-(butyldipentylstannyl)-1-methyl-1H-imidazole (340 mg, 0.92 mmol) and LiCl (78 mg, 1.8 mmol) in DMF (10 mL) was added Pd(PPh₃)₄ (71 mg, 0.06 mmol). The resulting mixture was stirred at 120° C. for 12 h and then the reaction mixture was filtered and the filtrate was concentrated in vacuo to give crude product: ethyl 6-methyl-5-(2-(1-methyl-1H-imidazol-4-yl)pyrazolo[5,1-b]thiazole-7-carboxamido)nicotinate (500 mg, crude). LCMS (ESI): mass calcd. for C₁₉H₁₈N₆O₃S, 410.4; m/z found, 411.2 [M+H]⁺.

### Step b: 6-methyl-5-(2-(1-methyl-1H-imidazol-4-yl)pyrazolo[5,1-b]thiazole-7-carboxamido)nicotinic acid

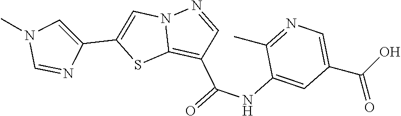 [SMILES: Cc1ncc(C(=O)O)cc1NC(=O)c1cnn2cc(-c3cn(C)cn3)sc12]

To a solution of ethyl 6-methyl-5-(2-(1-methyl-1H-imidazol-4-yl)pyrazolo[5,1-b]thiazole-7-carboxamido)nicotinate (500 mg, 1.2 mmol) in THF/MeOH/water (10 mL, 3/1/1) was added LiOH (59 mg, 2.5 mmol) at room temperature. The resulting mixture was stirred at 40° C. for 1 hour. The reaction mixture was adjusted to pH=3-4 with HCl (aq, 2 M). Then the mixture was filtered and washed with H₂O (20 mL×3). The solid was evaporated under vacuum to give the title compound 6-methyl-5-(2-(1-methyl-1H-imidazol-4-yl)pyrazolo[5,1-b]thiazole-7-carboxamido)nicotinic acid (400 mg, 46%) as a yellow solid. LCMS (ESI): mass calcd. for C₁₇H₁₄N₆O₃S, 382.4; m/z found, 383.0 [M+H]⁺.

### Step c: N-(5-((2-(3,3-dimethylazetidin-1-yl)ethyl)carbamoyl)-2-methylpyridin-3-yl)-2-(1-methyl-1H-imidazol-4-yl)pyrazolo[5,1-b]thiazole-7-carboxamide

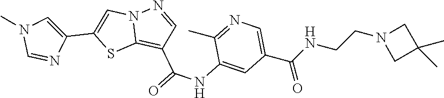

To a solution of 6-methyl-5-(2-(1-methyl-1H-imidazol-4-yl)pyrazolo[5,1-b]thiazole-7-carboxamido)nicotinic acid (200 mg, 0.44 mmol), HATU (337 mg, 0.89 mmol) and N,N-diisopropylethylamine (230 mg, 1.8 mmol) in DMF (2 mL) was added 2-(3,3-dimethylazetidin-1-yl)ethanamine (57 mg, 0.44 mmol). The resulting mixture was stirred at room temperature for 2 h and then concentrated under vacuum to give the crude product, which was purified by preparative high-performance liquid chromatography over column: Phenomenex Synergi C18 150*30 mm*4 um to give the title compound N-(5-((2-(5-azaspiro[2.4]heptan-5-yl)ethyl)carbamoyl)-2-methylpyridin-3-yl)-2-(1-(2-methoxyethyl)-1H-pyrazol-4-yl)pyrazolo[5,1-b]thiazole-7-carboxamide (25 mg, 11%) as a white solid. LCMS (ESI): mass calcd. for C₂₄H₂₈N₈O₂S, 492.6; m/z found, 493.5 [M+H]⁺. ¹H NMR (400 MHz, METHANOL-d₄) δ 8.80 (d, J=1.76 Hz, 1H), 8.43 (s, 1H), 8.33 (s, 1H), 8.29 (s, 1H), 7.73 (s, 1H), 7.59 (s, 1H), 3.81 (s, 3H), 3.43 (t, J=6.53 Hz, 2H), 3.14 (s, 4H), 2.75 (t, J=6.53 Hz, 2H), 2.63 (s, 3H), 1.26 (s, 6H).

### Example 162: N-(5-((2-(5-azaspiro[2.4]heptan-5-yl)ethyl)carbamoyl)-2-methylpyridin-3-yl)-2-(1-methyl-1H-imidazol-4-yl)pyrazolo[5,1-b]thiazole-7-carboxamide

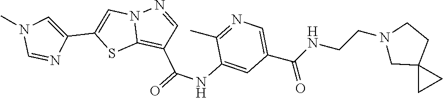 [SMILES: Cc1ncc(C(=O)NCCN2CCC3(CC3)C2)cc1NC(=O)c1cnn2cc(-c3cn(C)cn3)sc12]

To a solution of 5-(2-(1-(2-methoxyethyl)-1H-pyrazol-4-yl)pyrazolo[5,1-b]thiazole-7-carboxamido)-6-methylnicotinic acid (80 mg, 0.18 mmol), HATU (134 mg, 0.35 mmol) and N,N-diisopropylethylamine (91 mg, 0.71 mmol) in DMF (2 mL) was added 2-(5-azaspiro[2.4]heptan-5-yl)ethanamine (25 mg, 0.18 mmol). The resulting mixture was stirred at room temperature for 2 h and then concentrated under vacuum to give the crude product, which was purified by preparative high-performance liquid chromatography over column: Phenomenex Synergi C18 150*30 mm*4 um to give the title compound N-(5-((2-(5-azaspiro[2.4]heptan-5-yl)ethyl)carbamoyl)-2-methylpyridin-3-yl)-2-(1-methyl-1H-imidazol-4-yl)pyrazolo[5,1-b]thiazole-7-carboxamide (30 mg, 33%) as a white solid. LCMS (ESI): mass calcd. for C₂₅H₂₈N₈O₂S, 504.6; m/z found, 505.2 [M+H]⁺. ¹H NMR (400 MHz, METHANOL-d₄) δ 8.80 (d, J=1.91 Hz, 1H), 8.42 (s, 1H), 8.35 (d, J=1.67 Hz, 1H), 8.27 (s, 1H), 7.73 (s, 1H), 7.58 (s, 1H), 3.80 (s, 3H), 3.58 (t, J=6.74 Hz, 2H), 2.87 (t, J=7.03 Hz, 2H), 2.78 (t, J=6.74 Hz, 2H), 2.58-2.68 (m, 5H), 1.88 (t, J=7.03 Hz, 2H), 0.55-0.68 (m, 1H), 0.55-0.68 (m, 3H).

### Example 163: N-(5-((2-(2,2-dimethylpyrrolidin-1-yl)ethyl)carbamoyl)-2-methylpyridin-3-yl)-2-(1-methyl-1H-imidazol-4-yl)pyrazolo[5,1-b]thiazole-7-carboxamide

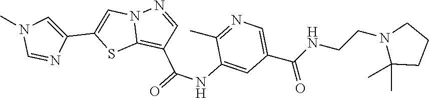 [SMILES: Cc1ncc(C(=O)NCCN2CCCC2(C)C)cc1NC(=O)c1cnn2cc(-c3cn(C)cn3)sc12]

To a solution of 5-(2-(1-(2-methoxyethyl)-1H-pyrazol-4-yl)pyrazolo[5,1-b]thiazole-7-carboxamido)-6-methylnicotinic acid (80 mg, 0.18 mmol), HATU (134 mg, 0.35 mmol) and N,N-diisopropylethylamine (91 mg, 0.71 mmol) in DMF (2 mL) was added 2-(2,2-dimethylpyrrolidin-1-yl)ethanamine (25 mg, 0.18 mmol). The resulting mixture was stirred at room temperature for 2 h and then concentrated under vacuum to give the crude product, which was purified by preparative high-performance liquid chromatography over column: Phenomenex Synergi C18 150*30 mm*4 um to give the title compound N-(5-((2-(2,2-dimethylpyrrolidin-1-yl)ethyl)carbamoyl)-2-methylpyridin-3-yl)-2-(1-methyl-1H-imidazol-4-yl)pyrazolo[5,1-b]thiazole-7-carboxamide (28.2 mg, 30%) as a white solid. LCMS (ESI): mass calcd. for C₂₅H₃₀N₈O₂S, 506.6; m/z found, 507.2 [M+H]⁺. ¹H NMR (400 MHz, METHANOL-d₄) δ 8.78 (d, J=1.76 Hz, 1H), 8.42 (s, 1H), 8.33 (d, J=2.01 Hz, 1H), 8.28 (s, 1H), 7.73 (s, 1H), 7.58 (s, 1H), 3.80 (s, 3H), 3.53 (br t, J=7.03 Hz, 2H), 2.92 (br t, J=7.28 Hz, 2H), 2.69 (br t, J=7.03 Hz, 2H), 2.62 (s, 3H), 1.78-1.87 (m, 2H), 1.68-1.74 (m, 2H), 1.06 (s, 6H).

### Example 164: N-(2-(2,6-dimethylpiperidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

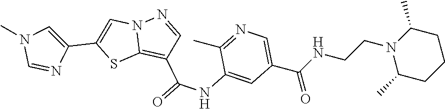 [SMILES: Cc1ncc(C(=O)NCCN2[C@H](C)CCC[C@@H]2C)cc1NC(=O)c1cnn2cc(-c3cn(C)cn3)sc12]

### Step a: 2-(2,6-dimethylpiperidin-1-yl)acetonitrile

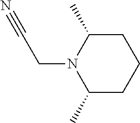

To a solution of 2,6-dimethylpiperidine (3 g, 26 mmol) and 2-bromoacetonitrile (2 mL, 29 mmol) in DMF (30 mL) was added K₂CO₃ (9 g, 66 mmol) at room temperature. The resulting mixture was stirred at 80° C. for 3 h. The reaction mixture was filtered. The filtrate was concentrated under reduced pressure to give crude product, which was purified by column chromatography over silica gel (eluent: eluent: PE/EA=100/0 to PE/EA=50/50) to give the title compound 2-(2,6-dimethylpiperidin-1-yl)acetonitrile (3.5 g, 87%) as a colorless oil. LCMS (ESI): mass calcd. for C₉H₁₆N₂, 152.2; m/z found, [M+H]⁺. ¹H NMR (400 MHz, METHANOL-d₄) δ 3.87 (s, 2H), 2.30-2.48 (m, 2H), 1.59-1.74 (m, 3H), 1.20-1.47 (m, 3H), 1.11 (s, 3H), 1.10 (s, 3H),

### Step b: 2-(2,6-dimethylpiperidin-1-yl)ethanamine

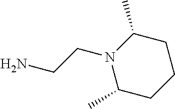

To a solution of 2-(2,6-dimethylpiperidin-1-yl)acetonitrile (3.5 g, 23 mmol) in THE (15 mL) was added LiAlH₄ (1 g, 27 mmol) by portions at 0° C. (ice/water), and the resulting mixture was stirred at 20° C. for 3 h. After cooled to 0° C., the reaction mixture was quenched with water (1 mL) and filtered. The filtrate was then concentrated to dryness under reduced pressure to afford the crude product 2-(2,6-dimethylpiperidin-1-yl)ethanamine (3.2 g, 86%) as a colorless oil. LCMS (ESI): mass calcd. for C₉H₂₀N₂, 156.2; m/z found, 157.4 [M+H]⁺. ¹HNMR (400 MHz, METHANOL-d₄) δ 2.62-2.77 (m, 4H) 2.48 (ddd, J=10.47, 6.39, 2.09 Hz, 2H), 1.62-1.72 (m, 1H), 1.50-1.60 (m, 2H), 1.19-1.43 (m, 3H), 1.13 (d, J=6.17 Hz, 6H).

### Step c: 5-amino-N-(2-(2,6-dimethylpiperidin-1-yl)ethyl)-6-methylnicotinamide

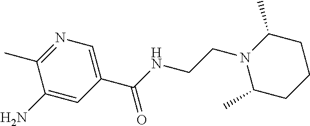 [SMILES: Cc1ncc(C(=O)NCCN2[C@H](C)CCC[C@@H]2C)cc1N]

To a solution of HATU (2.5 g, 6.6 mmol) was added to the 5-amino-6-methylnicotinic acid (500 mg, 3.3 mmol), and DIEA (2.2 mL, 13 mmol) in DMF (6 mL) was added 2-(2,6-dimethylpiperidin-1-yl)ethanamine (570 mg, 3.6 mmol). The mixture stirred at 35° C. for 16 hours and then concentrated under vacuum to give the crude product, which was purified by column chromatography over silica gel (eluent: eluent: Dichlormethane/Methanol=100/0 to Dichlormethane/Methanol=20/80) to give pure product 5-amino-N-(2-(2,6-dimethylpiperidin-1-yl)ethyl)-6-methylnicotinamide (280 mg, 26%) as a yellow oil. LCMS (ESI): mass calcd. for C₁₆H₂₆N₄O, 290.4; m/z found, 291.3 [M+H]⁺. ¹H NMR (400 MHz, METHANOL-d₄) δ 8.12 (br s, 1H), 7.43 (s, 1H), 3.69 (br s, 2H), 3.45 (br s, 2H), 3.33 (s, 2H), 2.39 (s, 3H), 1.29 (br d, J=17.64 Hz, 2H) 1.95-2.06 (m, 2H), 1.81 (br s, 2H), 1.62 (br d, J=9.26 Hz, 2H), 1.42-1.52 (m, 6H).

### Step d: N-(2-(2,6-dimethylpiperidin-1-yl)ethyl)-6-methyl-5-((1-methyl-6-((1-methyl-11H-pyrazol-4-yl)amino)-1H-pyrazolo[3,4-d]pyrimidin-3-yl)amino)nicotinamide

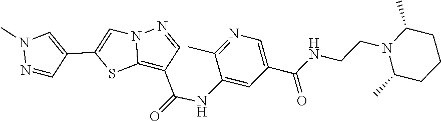 [SMILES: Cc1ncc(C(=O)NCCN2[C@H](C)CCC[C@@H]2C)cc1NC(=O)c1cnn2cc(-c3cnn(C)c3)sc12]

To the mixture of 1-methyl-6-((1-methyl-1H-pyrazol-4-yl)amino)-1H-pyrazolo[3,4-d]pyrimidine-3-carboxylic acid (100 mg, 0.4 mmol) and 5-amino-N-(2-(2,6-dimethylpiperidin-1-yl)ethyl)-6-methylnicotinamide (175 mg, 0.6 mmol) and POCl₃ (75 μL, 0.8 mmol), then was added Pyridine (5 mL). After stirred at 0° C. for ˜1 h, the 10 mL of sat. aq. NaHCO₃ was added, and the mixture was extracted with DCM (10 mL*4). The combined organic layer was washed with brine (10 mL), water (10 mL), dried under Na₂SO₄ and filtered to give the crude product, which was purified by preparative high-performance liquid chromatography over column: Phenomenex Gemini-NX 80*40 mm*3 um and by Supercritical Fluid Chromatography over column: DAICEL CHIRALPAK AD (250 mm*30 mm, 10 um) to give the title compound N-(2-(2,6-dimethylpiperidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (10 mg, 6%) as a white solid. LCMS (ESI): mass calcd. for C₂₆H₃₂N₈O₂S, 520.6; m/z found, 521.3 [M+H]⁺. ¹H NMR (400 MHz, METHANOL-d₄) δ 8.68 (d, J=1.98 Hz, 1H), 8.32 (s, 1H), 8.23 (s, 1H), 8.15 (s, 1H), 7.94 (s, 1H), 7.73 (s, 1H), 7.70-7.76 (m, 1H), 3.85 (s, 3H), 3.56 (br s, 3H), 3.26-3.32 (m, 3H), 2.52 (s, 3H), 1.71 (br s, 3H), 1.40 (br s, 5H), 1.30 (br s, 6H).

### Example 165: N-(5-((2-(4,4-difluoropiperidin-1-yl)ethyl)carbamoyl)-2-methylpyridin-3-yl)-2-(1-(2-methoxyethyl)-1H-pyrazol-4-yl)pyrazolo[5,1-b]thiazole-7-carboxamide

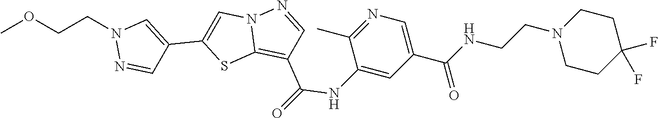 [SMILES: COCCn1cc(-c2cn3ncc(C(=O)Nc4cc(C(=O)NCCN5CCC(F)(F)CC5)cnc4C)c3s2)cn1]

To a solution of 5-(2-(1-(2-methoxyethyl)-1H-pyrazol-4-yl)pyrazolo[5,1-b]thiazole-7-carboxamido)-6-methylnicotinic acid (150 mg, 0.35 mmol), 2-(4,4-difluoropiperidin-1-yl)ethanamine (63.5 mg, 0.39 mmol) and N,N-diisopropylethylamine (136 mg, 1.06 mmol) in N,N-dimethylformamide (5 mL) was added 2-(3H-[1,2,3]triazolo[4,5-b]pyridin-3-yl)-1,1,3,3-tetramethylisouronium hexafluorophosphate(V) (160 mg, 0.42 mmol). The mixture stirred at room temperature for 2 h and then concentrated under vacuum to give the crude product, which was purified by preparative high-performance liquid chromatography over column: Welch Xtimate C18 150*30 mm*5 um to give the title compound N-(5-((2-(4,4-difluoropiperidin-1-yl)ethyl)carbamoyl)-2-methylpyridin-3-yl)-2-(1-(2-methoxyethyl)-1H-pyrazol-4-yl)pyrazolo[5,1-b]thiazole-7-carboxamide (52.3 mg, 25%) as a yellow solid. LCMS (ESI): mass calcd. for C₂₆H₃₀F₂N₈O₃S, 572.63; m/z found, 573.3[M+H]⁺. ¹H NMR (400 MHz, METHANOL-d₄) δ 8.77 (d, J=2.0 Hz, 1H), 8.41 (s, 1H), 8.31 (d, J=2.0 Hz, 1H), 8.25 (s, 1H), 8.07 (s, 1H), 7.86 (s, 1H), 4.35 (t, J=5.1 Hz, 2H), 3.77 (t, J=5.2 Hz, 2H), 3.56 (t, J=6.6 Hz, 2H), 3.34 (s, 3H), 2.69-2.65 (m, 6H), 2.61 (s, 3H), 2.04-1.97 (m, 4H).

### Example 166: N-(5-((2-(3,3-dimethylazetidin-1-yl)ethyl)carbamoyl)-2-methylpyridin-3-yl)-2-(1-(2-methoxyethyl)-1H-pyrazol-4-yl)pyrazolo[5,1-b]thiazole-7-carboxamide

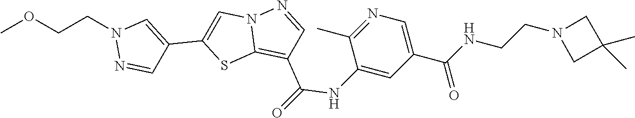

To a solution of 5-(2-(1-(2-methoxyethyl)-1H-pyrazol-4-yl)pyrazolo[5,1-b]thiazole-7-carboxamido)-6-methylnicotinic acid (100 mg, 0.23 mmol), 2-(3,3-dimethylazetidin-1-yl)ethanamine (33.1 mg, 0.26 mmol) and N,N-diisopropylethylamine (90.9 mg, 0.70 mmol) in N,N-dimethylformamide (5 mL) was added 2-(3H-[1,2,3]triazolo[4,5-b]pyridin-3-yl)-1,1,3,3-tetramethylisouronium hexafluorophosphate(V) (107 mg, 0.28 mmol). The mixture stirred at room temperature for 2 h and then concentrated under vacuum to give the crude product, which was purified by preparative high-performance liquid chromatography over column: Welch Xtimate C18 150*30 mm*5 um to give the title compound N-(5-((2-(3,3-dimethylazetidin-1-yl)ethyl)carbamoyl)-2-methylpyridin-3-yl)-2-(1-(2-methoxyethyl)-1H-pyrazol-4-yl)pyrazolo[5,1-b]thiazole-7-carboxamide (50 mg, 30%) as a yellow solid. LCMS (ESI): mass calcd. for C₂₆H₃₂N₈O₃S, 536.649; m/z found, 537.2[M+H]⁺. ¹H NMR (400 MHz, METHANOL-d₄) δ 8.78 (d, J=2.0 Hz, 1H), 8.41 (s, 1H), 8.31 (d, J=1.8 Hz, 1H), 8.26 (s, 1H), 8.07 (s, 1H), 7.86 (s, 1H), 4.35 (t, J=5.1 Hz, 2H), 3.77 (t, J=5.1 Hz, 2H), 3.43-3.40 (m, 2H), 3.34 (s, 3H), 3.12 (s, 4H), 2.74 (t, J=6.6 Hz, 2H), 2.61 (s, 3H), 1.24 (s, 6H).

### Example 167: N-(5-((2-(3,3-difluoropyrrolidin-1-yl)ethyl)carbamoyl)-2-methylpyridin-3-yl)-2-(1-(2-methoxyethyl)-1H-pyrazol-4-yl)pyrazolo[5,1-b]thiazole-7-carboxamide

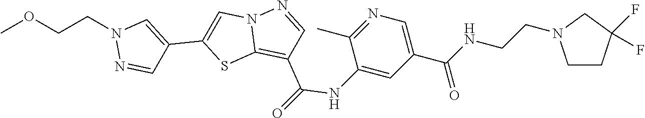 [SMILES: COCCn1cc(-c2cn3ncc(C(=O)Nc4cc(C(=O)NCCN5CCC(F)(F)C5)cnc4C)c3s2)cn1]

To a solution of 5-(2-(1-(2-methoxyethyl)-1H-pyrazol-4-yl)pyrazolo[5,1-b]thiazole-7-carboxamido)-6-methylnicotinic acid (150 mg, 0.35 mmol), 2-(3,3-difluoropyrrolidin-1-yl)ethanamine (58.1 mg, 0.39 mmol) and N,N-diisopropylethylamine (136 mg, 1.1 mmol) in N,N-dimethylformamide (6 mL) was added 2-(3H-[1,2,3]triazolo[4,5-b]pyridin-3-yl)-1,1,3,3-tetramethylisouronium hexafluorophosphate(V) (160 mg, 0.42 mmol). The mixture stirred at room temperature for 2 h and then concentrated under vacuum to give the crude product, which was purified by preparative high-performance liquid chromatography over column: Welch Xtimate C18 150*30 mm*5 um to give the title compound N-(5-((2-(3,3-difluoropyrrolidin-1-yl)ethyl)carbamoyl)-2-methylpyridin-3-yl)-2-(1-(2-methoxyethyl)-1H-pyrazol-4-yl)pyrazolo[5,1-b]thiazole-7-carboxamide (69.6 mg, 35%) as a white solid. LCMS (ESI): mass calcd. for C₂₅H₂₈F₂N₈O₃S, 558.603; m/z found, 559.3[M+H]⁺. ¹H NMR (400 MHz, METHANOL-d₄) δ 8.76 (d, J=2.0 Hz, 1H), 8.39 (s, 1H), 8.28 (d, J=1.8 Hz, 1H), 8.24 (s, 1H), 8.05 (s, 1H), 7.84 (s, 1H), 4.33 (t, J=5.1 Hz, 2H), 3.75 (t, J=5.1 Hz, 2H), 3.52 (t, J=6.5 Hz, 2H), 3.32 (s, 3H), 2.98 (t, J=13.2 Hz, 2H), 2.82 (t, J=7.1 Hz, 2H), 2.72 (t, J=6.5 Hz, 2H), 2.59 (s, 3H), 2.31-2.21 (m, 2H).

### Example 168: N-(5-((2-(2-azaspiro[3.3]heptan-2-yl)ethyl)carbamoyl)-2-methylpyridin-3-yl)-2-(1-(2-methoxyethyl)-1H-pyrazol-4-yl)pyrazolo[5,1-b]thiazole-7-carboxamide

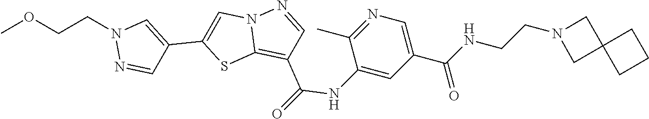 [SMILES: COCCn1cc(-c2cn3ncc(C(=O)Nc4cc(C(=O)NCCN5CC6(CCC6)C5)cnc4C)c3s2)cn1]

To a solution of 5-(2-(1-(2-methoxyethyl)-1H-pyrazol-4-yl)pyrazolo[5,1-b]thiazole-7-carboxamido)-6-methylnicotinic acid (150 mg, 0.35 mmol), 2-(2-azaspiro[3.3]heptan-2-yl)ethanamine (54.2 mg, 0.39 mmol) and N,N-diisopropylethylamine (136 mg, 1.1 mmol) in N,N-dimethylformamide (5 mL) was added 2-(3H-[1,2,3]triazolo[4,5-b]pyridin-3-yl)-1,1,3,3-tetramethylisouronium hexafluorophosphate(V) (160 mg, 0.42 mmol). The mixture stirred at room temperature for 2 h and then concentrated under vacuum to give the crude product, which was purified by preparative high-performance liquid chromatography over column: Welch Xtimate C18 150*30 mm*5 um to give the title compound N-(5-((2-(2-azaspiro[3.3]heptan-2-yl)ethyl)carbamoyl)-2-methylpyridin-3-yl)-2-(1-(2-methoxyethyl)-1H-pyrazol-4-yl)pyrazolo[5,1-b]thiazole-7-carboxamide (33.5 mg, 17%) as a white solid. LCMS (ESI): mass calcd. for C₂₇H₃₂N₈O₃S, 548.66; m/z found, 549.3[M+H]⁺. ¹H NMR (400 MHz, METHANOL-d₄) δ 8.78 (d, J=2.0 Hz, 1H), 8.41 (s, 1H), 8.31 (d, J=2.0 Hz, 1H), 8.26 (s, 1H), 8.07 (s, 1H), 7.86 (s, 1H), 4.35 (t, J=5.1 Hz, 2H), 3.77 (t, J=5.2 Hz, 2H), 3.40 (t, J=6.5 Hz, 2H), 3.34 (s, 3H), 3.33-3.32 (m, 4H), 2.67 (br t, J=6.2 Hz, 2H), 2.61 (s, 3H), 2.14 (t, J=7.6 Hz, 4H), 1.88-1.78 (m, 2H).

### Example 169: N-(5-((2-(1-azaspiro[3.3]heptan-1-yl)ethyl)carbamoyl)-2-methylpyridin-3-yl)-2-(1,5-dimethyl-1H-pyrazol-4-yl)pyrazolo[5,1-b]thiazole-7-carboxamide

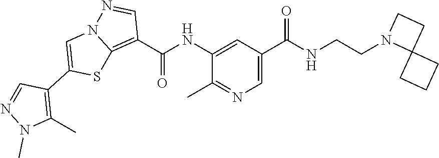 [SMILES: Cc1ncc(C(=O)NCCN2CCC23CCC3)cc1NC(=O)c1cnn2cc(-c3cnn(C)c3C)sc12]

### Step a: 2-(1-azaspiro[3.3]heptan-1-yl)acetonitrile

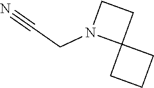

To the mixture of 1-azaspiro[3.3]heptane hemioxalate (500 mg, 1.8 mmol) in MeCN (10 mL) was added 2-bromoacetonitrile (0.15 mL, 2.2 mmol) and K₂CO₃ (550 mg, 4.0 mmol) at 25° C. and stirred for 6 hours. The resulting mixture was quenched with water (5 mL) and extracted with ethyl acetate (10 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=4:1) to give the title compound 2-(1-azaspiro[3.3]heptan-1-yl)acetonitrile (390 mg, 81%) as a yellow oil. ¹H NMR (400 MHz, CHLOROFORM-d) δ 3.46 (s, 2H), 3.36 (t, J=7.03 Hz, 2H), 2.20-2.34 (m, 4H), 1.98-2.08 (m, 2H), 1.62-1.72 (m, 2H)

### Step b: 2-(1-azaspiro[3.3]heptan-1-yl)ethanamine

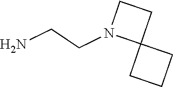

To a solution of 2-(4-azaspiro[2.4]heptan-4-yl)acetonitrile (220 mg, 1.62 mmol) in THE (6 mL) was added lithium aluminium hydride (67 mg, 1.78 mmol) by portions at 0° C. (ice/water). The resultant mixture was stirred at 20° C. for 90 min before quenched with water (100 mg) at 0° C. The reaction mixture was filtered. And the filtrate was concentrated to dryness under reduced pressure to afford the crude product 2-(1-azaspiro[3.3]heptan-1-yl)ethanamine (270 mg, 71%) as a yellow oil which was used to the next step without further purification. ¹H NMR (400 MHz, CHLOROFORM-d) δ 3.03 (t, J=6.85 Hz, 2H), 2.60-2.67 (m, 2H), 2.40-2.49 (m, 2H), 2.06-2.15 (m, 4H), 1.82-1.88 (m, 2H), 1.51-1.55 (m, 2H).

### Step c: methyl 5-(2-(1,5-dimethyl-1H-pyrazol-4-yl)pyrazolo[5,1-b]thiazole-7-carboxamido)-6-methylnicotinate

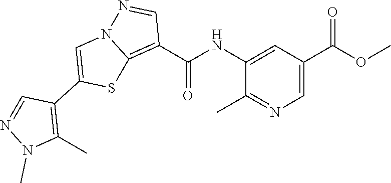 [SMILES: COC(=O)c1cnc(C)c(NC(=O)c2cnn3cc(-c4cnn(C)c4C)sc23)c1]

To the mixture of methyl 5-(2-bromopyrazolo[5,1-b]thiazole-7-carboxamido)-6-methylnicotinate (1.5 g, 3.8 mmol) in 1,4-dioxane/H₂O=4:1 (200 mL) was added 1,5-dimethyl-4-(4,4,5,5-tetramethyl-1,3,2-dioxaborolan-2-yl)-1H-pyrazole (1.1 g, 5.0 mmol) and Cs₂CO₃ (3.8 g, 12 mmol), then Pd(dppf)Cl₂·CH₂Cl₂ (940 mg, 1.2 mmol) was added under N₂. The resulting mixture was stirred at 100° C. for 12 h before cooled to 25° C. Then the reaction mixture was concentrated under reduced pressure to afford the crude product methyl 5-(2-(1,5-dimethyl-1H-pyrazol-4-yl)pyrazolo[5,1-b]thiazole-7-carboxamido)-6-methylnicotinate (1.5 g, 11%) as a black solid. LCMS (ESI): mass calcd. for C₁₈H₁₆N₆O₃S, 410.4; m/z found, 411.1 [M+H]⁺.

### Step d: 5-(2-(1,5-dimethyl-1H-pyrazol-4-yl)pyrazolo[5,1-b]thiazole-7-carboxamido)-6-methylnicotinic acid

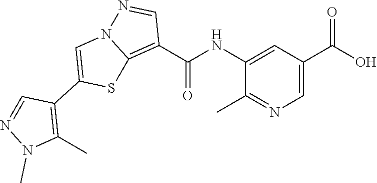 [SMILES: Cc1ncc(C(=O)O)cc1NC(=O)c1cnn2cc(-c3cnn(C)c3C)sc12]

To a solution of 5-(2-(1,5-dimethyl-1H-pyrazol-4-yl)pyrazolo[5,1-b]thiazole-7-carboxamido)-6-methylnicotinic acid and methyl 5-(2-(1,5-dimethyl-1H-pyrazol-4-yl)pyrazolo[5,1-b]thiazole-7-carboxamido)-6-methylnicotinate (2.8 g, 3.5 mmol) in THF/MeOH=1/1 (120 mL) was added lithium hydroxide hydrate (150 mg, 3.6 mmol) in H₂O (60 mL) and the reaction was stirred at 20° C. for 1 hour. The reaction mixture was stirred at 50° C. for 2 h before cooling to room temperature. The mixture was adjusted to pH=5-6 with HCl (aq, 2 M). The mixture was filtered and washed with water (10 mL×3). The solid was evaporated under vacuum to give desired product 5-(2-(1,5-dimethyl-1H-pyrazol-4-yl)pyrazolo[5,1-b]thiazole-7-carboxamido)-6-methylnicotinic acid (3.2 g, 70%) as white solid. LCMS (ESI): mass calcd. for C₁₈H₁₆N₆O₃S, 396.4; m/z found, 397.2 [M+H]⁺.

### Step e: N-(5-((2-(1-azaspiro[3.3]heptan-1-yl)ethyl)carbamoyl)-2-methylpyridin-3-yl)-2-(1,5-dimethyl-1H-pyrazol-4-yl)pyrazolo[5,1-b]thiazole-7-carboxamide

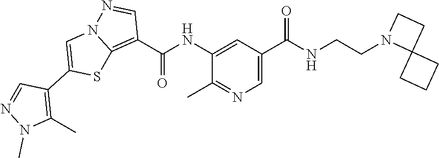 [SMILES: Cc1ncc(C(=O)NCCN2CCC23CCC3)cc1NC(=O)c1cnn2cc(-c3cnn(C)c3C)sc12]

To the mixture of 5-(2-(1,5-dimethyl-1H-pyrazol-4-yl)pyrazolo[5,1-b]thiazole-7-carboxamido)-6-methylnicotinic acid (240 mg, 0.37 mmol) in DMF (8 mL) was added 2-(1-azaspiro[3.3]heptan-1-yl)ethanamine (64 mg, 0.46 mmol) and DIEA (280 μL, 1.7 mmol), then HATU (184 mg, 0.48 mmol) was added. The resulting mixture was stirred at 25° C. for 2 hours and then concentrated under vacuum to give the crude product, which was purified by preparative high-performance liquid chromatography over column: Phenomenex C18 80*40 mm*3 um to give the title compound N-(5-((2-(1-azaspiro[3.3]heptan-1-yl)ethyl)carbamoyl)-2-methylpyridin-3-yl)-2-(1,5-dimethyl-1H-pyrazol-4-yl)pyrazolo[5,1-b]thiazole-7-carboxamide (31 mg, 16%) as a white solid. LCMS (ESI): mass calcd. for C₂₆H₃₀N₈O₂S, 518.6; m/z found, 519.3 [M+H]⁺. ¹H NMR (400 MHz, METHANOL-d₄) δ 8.81 (d, J=1.76 Hz, 1H), 8.45 (s, 1H), 8.33 (d, J=2.01 Hz, 1H), 8.15 (s, 1H), 7.70 (s, 1H), 3.88 (s, 3H), 3.47 (t, J=6.90 Hz, 2H), 3.13-3.28 (m, 2H), 2.75 (t, J=6.90 Hz, 2H), 2.63 (s, 3H), 2.51 (s, 3H), 2.30-2.41 (m, 2H), 2.26 (t, J=7.03 Hz, 2H), 2.00 (br d, J=6.27 Hz, 2H), 1.63-1.75 (m, 2H).

### Example 170: N-(5-((2-(5-azaspiro[2.4]heptan-5-yl)ethyl)carbamoyl)-2-methylpyridin-3-yl)-2-(1,5-dimethyl-1H-pyrazol-4-yl)pyrazolo[5,1-b]thiazole-7-carboxamide

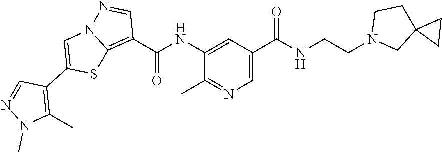 [SMILES: Cc1ncc(C(=O)NCCN2CCC3(CC3)C2)cc1NC(=O)c1cnn2cc(-c3cnn(C)c3C)sc12]

To a solution of 5-(2-(1,5-dimethyl-1H-pyrazol-4-yl)pyrazolo[5,1-b]thiazole-7-carboxamido)-6-methylnicotinic acid (240 mg, 0.37 mmol) in DMF (8 mL) was added 2-(5-azaspiro[2.4]heptan-5-yl)ethanamine (80 mg, 0.57 mmol) and DIEA (0.25 mL, 1.5 mmol), then HATU (180 mg, 0.48 mmol) was added at room temperature. The resulting mixture was stirred at room temperature for 2 h before cooling to room temperature. The reaction mixture was filtered and the filtrate was concentrated under vacuum to give crude product, which was purified by preparative high-performance liquid chromatography over column: Phenomenex C18 80*40 mm*3 um to give the title compound N-(5-((2-(5-azaspiro[2.4]heptan-5-yl)ethyl)carbamoyl)-2-methylpyridin-3-yl)-2-(1,5-dimethyl-1H-pyrazol-4-yl)pyrazolo[5,1-b]thiazole-7-carboxamide (33 mg, 17%) as a white solid. LCMS (ESI): mass calcd. for C₂₆H₃₀N₈O₂S 518.6; m/z found, 519.3 [M+H]⁺. ¹H NMR (400 MHz, METHANOL-d₄) δ 8.82 (d, J=2.15 Hz, 1H), 8.45 (s, 1H), 8.35 (d, J=2.03 Hz, 1H), 8.14 (s, 1H), 7.70 (s, 1H), 3.88 (s, 3H), 3.61 (t, J=6.62 Hz, 2H), 2.91-2.98 (m, 2H), 2.84 (t, J=6.68 Hz, 2H), 2.71 (s, 2H), 2.63 (s, 3H), 2.50 (s, 3H), 1.90 (t, J=7.09 Hz, 2H), 0.60-0.66 (m, 4H).

### Example 171: N-(5-((2-(2-azabicyclo[2.2.2]octan-2-yl)ethyl)carbamoyl)-2-methylpyridin-3-yl)-2-(1,5-dimethyl-1H-pyrazol-4-yl)pyrazolo[5,1-b]thiazole-7-carboxamide

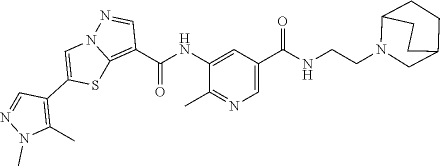 [SMILES: Cc1ncc(C(=O)NCCN2CC3CCC2CC3)cc1NC(=O)c1cnn2cc(-c3cnn(C)c3C)sc12]

To the mixture of 5-(2-(1,5-dimethyl-1H-pyrazol-4-yl)pyrazolo[5,1-b]thiazole-7-carboxamido)-6-methylnicotinic acid (90 mg, 0.19 mmol) in DMF (4 mL) was added 2-(2-azabicyclo[2.2.2]octan-2-yl)ethanamine (36 mg, 0.23 mmol) and DIEA (0.13 mL, 0.79 mmol), then HATU (90 mg, 0.24 mmol) was added. The mixture stirred at 25° C. for 2 hours and then concentrated under vacuum to give the crude product, which was purified by preparative high-performance liquid chromatography over column: Phenomenex C18 150*40 mm*5 um and by Supercritical Fluid Chromatography over column: DAICEL CHIRALPAK AD (250 mm*30 mm, 10 um) to give the title compound N-(5-((2-(2-azabicyclo[2.2.2]octan-2-yl)ethyl)carbamoyl)-2-methylpyridin-3-yl)-2-(1,5-dimethyl-1H-pyrazol-4-yl)pyrazolo[5,1-b]thiazole-7-carboxamide (25 mg, 35%) as a white solid. LCMS (ESI): mass calcd. for C₂₇H₃₂N₈O₂S, 532.7; m/z found, 533.3 [M+H]⁺. ¹H NMR (400 MHz, METHANOL-d₄) δ 8.81 (d, J=1.8 Hz, 1H), 8.46 (s, 1H), 8.36 (d, J=1.8 Hz, 1H), 8.15 (d, J=1.0 Hz, 1H), 7.70 (s, 1H), 3.88 (s, 3H), 3.66 (br t, J=6.4 Hz, 2H), 3.11 (br s, 5H), 2.64 (s, 3H), 2.51 (s, 3H), 2.10 (br s, 2H), 1.83 (br s, 1H), 1.79-1.67 (m, 6H).

### Example 172: N-(5-((2-(7-azabicyclo[2.2.1]heptan-7-yl)ethyl)carbamoyl)-2-methylpyridin-3-yl)-2-(1,5-dimethyl-1H-pyrazol-4-yl)pyrazolo[5,1-b]thiazole-7-carboxamide

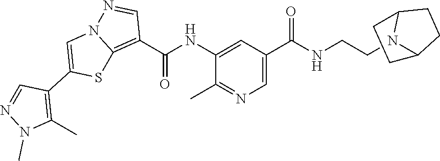 [SMILES: Cc1ncc(C(=O)NCCN2C3CCC2CC3)cc1NC(=O)c1cnn2cc(-c3cnn(C)c3C)sc12]

### Step a: 2-(7-azabicyclo[2.2.1]heptan-7-yl)acetonitrile

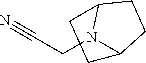

To a solution of 7-azabicyclo[2.2.1]heptane; hydrochloride (0.5 g, 3.7 mmol) and potassium carbonate (1.1 g, 8.2 mmol) in MeCN (6 mL) was added 2-bromoacetonitrile (0.28 mL, 4.5 mmol) at room temperature. The resulting mixture was stirred at 50° C. overnight. The reaction mixture was filtered. The combined filtrate was concentrated under reduced pressure to give crude product, which was purified by column chromatography over silica gel (eluent: petroleum ether:ethyl acetate=4:1) to give the title compound title compound 2-(7-azabicyclo[2.2.1]heptan-7-yl)acetonitrile (440 mg, 80%) as a colorless oil.

### Step b: 2-(7-azabicyclo[2.2.1]heptan-7-yl)ethanamine

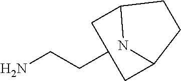 [SMILES: NCCC1CC2CCC(C1)N2]

To a solution of 2-(7-azabicyclo[2.2.1]heptan-7-yl)acetonitrile (440 mg, 3.2 mmol) in THE (15 mL) was added lithium aluminium hydride (168 mg, 4.4 mmol) by portions at 0° C. (ice/water), and the resulting mixture was stirred at 25° C. for 1 h. After cooled to 0° C., the reaction mixture was quenched with water (0.17 mL) and filtered. The filtrate was then concentrated to dryness under reduced pressure to afford the crude product 2-(7-azabicyclo[2.2.1]heptan-7-yl)ethanam (300 mg, 75%) as oil.

### Step c: N-(5-((2-(7-azabicyclo[2.2.1]heptan-7-yl)ethyl)carbamoyl)-2-methylpyridin-3-yl)-2-(1,5-dimethyl-1H-pyrazol-4-yl)pyrazolo[5,1-b]thiazole-7-carboxamide

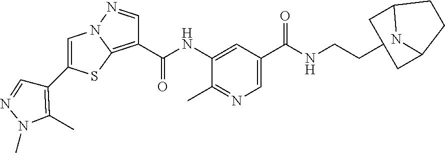 [SMILES: Cc1ncc(C(=O)NCCC2CC3CCC(C2)N3)cc1NC(=O)c1cnn2cc(-c3cnn(C)c3C)sc12]

To a solution of 5-((6-((1,3-dimethyl-1H-pyrazol-4-yl)amino)-1-methyl-1H-pyrazolo[3,4-d]pyrimidin-3-yl)amino)-6-methylnicotinic acid (150 mg, 0.23 mmol) in DMF (5 mL) was added 2-(3,3-dimethylpyrrolidin-1-yl)ethanamine (90 mg, 0.64 mmol) and DIEA (0.39 mL, 2.4 mmol), then HATU (270 mg, 0.71 mmol) was added. The mixture stirred at room temperature for 2 h and then concentrated under vacuum to give the crude product, which was purified by column chromatography over silica gel (eluent: DCM/MeOH from 100:0 to 80:20) to give the crude product, which was purified by preparative high-performance liquid chromatography over column: Welch Xtimate C18 150*30 mm*5 um to give the title compound N-(5-((2-(7-azabicyclo[2.2.1]heptan-7-yl)ethyl)carbamoyl)-2-methylpyridin-3-yl)-2-(1,5-dimethyl-1H-pyrazol-4-yl)pyrazolo[5,1-b]thiazole-7-carboxamide (23.7 mg, 40%) as a pale white solid. LCMS (ESI): mass calcd. for C₂₆H₃₀N₈O₂S, 518.6 m/z found, 519.2 [M+H]⁺. ¹H NMR (400 MHz, CHLOROFORM-d) δ 8.80 (d, J=1.91 Hz, 1H), 8.69 (d, J=1.79 Hz, 1H), 8.13 (s, 1H), 7.76 (s, 1H), 7.61 (s, 1H), 7.57 (br d, J=2.15 Hz, 1H), 7.55 (br d, J=5.96 Hz, 1H), 3.89 (s, 3H), 3.56 (q, J=5.72 Hz, 2H), 3.36 (br s, 2H), 2.67 (s, 3H), 2.66 (br s, 1H), 2.64 (br s, 1H), 2.47 (s, 3H), 1.76-1.83 (m, 3H), 1.76-1.77 (m, 1H), 1.36 (br d, J=7.15 Hz, 4H).

### Example 173: 2-(1,5-dimethyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide

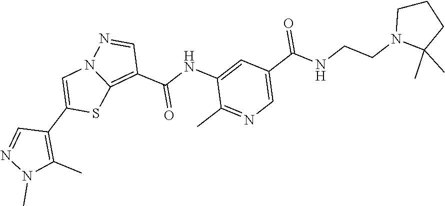 [SMILES: Cc1ncc(C(=O)NCCN2CCCC2(C)C)cc1NC(=O)c1cnn2cc(-c3cnn(C)c3C)sc12]

To a solution of 5-(2-(1,5-dimethyl-1H-pyrazol-4-yl)pyrazolo[5,1-b]thiazole-7-carboxamido)-6-methylnicotinic acid (100 mg, 0.19 mmol), HATU (110 mg, 0.29 mmol) and N,N-diisopropylethylamine (0.15 mL, 0.86 mmol) in N,N-dimethylformamide (5 mL) was added 2-(2,2-dimethylpyrrolidin-1-yl)ethanamine (40 mg, 0.28 mmol). The mixture stirred at room temperature for 1 hour and then concentrated under vacuum to give the crude product, which was purified by preparative high-performance liquid chromatography over column: Phenomenex Gemini-NX C18 75*30 mm*3 um to give the title compound 2-(1,5-dimethyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide (48 mg, 48%) as a white solid. LCMS (ESI): mass calcd. for C₂₆H₃₂N₈O₂S, 520.2; m/z found, 521.3 [M+H]⁺. ¹H NMR (400 MHz, METHANOL-d₄) δ 8.81 (d, J=2.0 Hz, 1H), 8.45 (s, 1H), 8.36 (d, J=2.0 Hz, 1H), 8.14 (s, 1H), 7.69 (s, 1H), 3.88 (s, 3H), 3.72-3.60 (m, 2H), 3.24 (br s, 2H), 2.98 (br s, 2H), 2.64 (s, 3H), 2.50 (s, 3H), 2.06-1.94 (m, 2H), 1.92-1.82 (m, 2H), 1.22 (s, 6H).

### Example 174: 2-(1,3-dimethyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide

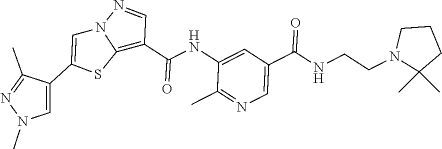 [SMILES: Cc1ncc(C(=O)NCCN2CCCC2(C)C)cc1NC(=O)c1cnn2cc(-c3cn(C)nc3C)sc12]

To the mixture of 5-(2-(1,3-dimethyl-1H-pyrazol-4-yl)pyrazolo[5,1-b]thiazole-7-carboxamido)-6-methylnicotinic acid (500 mg, 1.3 mmol) in DMF (25 mL) was added 2-(2-azabicyclo[2.2.2]octan-2-yl)ethanamine (150 mg, 1.1 mmol) and DIEA (550 μL, 3.3 mmol), then HATU (350 mg, 0.92 mmol) was added. The mixture stirred at 25° C. for 2 hours and then concentrated under vacuum to give the crude product, which was purified by preparative high-performance liquid chromatography over column: Phenomenex C18 80*40 mm*3 um to give the title compound 2-(1,3-dimethyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide (68 mg, 10%) as a white solid. LCMS (ESI): mass calcd. for C₂₆H₃₂N₈O₂S, 520.7; m/z found, 521.3 [M+H]⁺. ¹H NMR (400 MHz, METHANOL-d₄) δ 8.79 (d, J=2.0 Hz, 1H), 8.44 (s, 1H), 8.32 (d, J=1.8 Hz, 1H), 8.13 (s, 1H), 7.94 (s, 1H), 3.89 (s, 3H), 3.54 (t, J=7.0 Hz, 2H), 2.91 (br t, J=7.3 Hz, 2H), 2.69 (br t, J=7.0 Hz, 2H), 2.63 (s, 3H), 2.43 (s, 3H), 1.90-1.79 (m, 2H), 1.74-1.67 (m, 2H), 1.06 (s, 6H).

### Example 175: N-(5-((2-(1-azaspiro[3.3]heptan-1-yl)ethyl)carbamoyl)-2-methylpyridin-3-yl)-2-(1,3-dimethyl-1H-pyrazol-4-yl)pyrazolo[5,1-b]thiazole-7-carboxamide

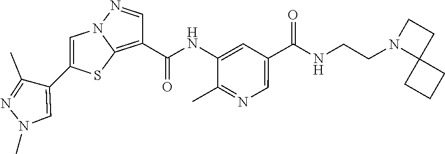 [SMILES: Cc1ncc(C(=O)NCCN2CCC23CCC3)cc1NC(=O)c1cnn2cc(-c3cn(C)nc3C)sc12]

To the mixture of 5-(2-(1,3-dimethyl-1H-pyrazol-4-yl)pyrazolo[5,1-b]thiazole-7-carboxamido)-6-methylnicotinic acid (230 mg, 0.58 mmol) in DMF (10 mL) was added 2-(1-azaspiro[3.3]heptan-1-yl)ethanamine (104 mg, 0.74 mmol) and DIEA (403 L, 2.4 mmol), then HATU (196 mg, 0.52 mmol) was added. The mixture stirred at 25° C. for 2 hours and then concentrated under vacuum to give the crude product, which was purified by preparative high-performance liquid chromatography over column: Welch Xtimate C18 100*40 mm*3 um to give the title compound N-(5-((2-(1-azaspiro[3.3]heptan-1-yl)ethyl)carbamoyl)-2-methylpyridin-3-yl)-2-(1,3-dimethyl-1H-pyrazol-4-yl)pyrazolo[5,1-b]thiazole-7-carboxamide (47 mg, 15%) as a white solid. LCMS (ESI): mass calcd. for C₂₆H₃₀N₈O₂S, 518.6; m/z found, 519.3 [M+H]⁺. ¹H NMR (400 MHz, METHANOL-d₄) δ 8.86-8.80 (m, 1H), 8.49-8.40 (m, 2H), 8.15 (s, 1H), 7.94 (s, 1H), 4.26-4.14 (m, 1H), 3.99 (q, J=9.8 Hz, 1H), 3.89 (s, 3H), 3.82-3.61 (m, 2H), 3.54-3.43 (m, 1H), 3.27 (br dd, J=6.1, 12.8 Hz, 1H), 2.88-2.72 (m, 2H), 2.71 (br d, J=7.7 Hz, 1H), 2.67 (s, 3H), 2.65-2.55 (m, 1H), 2.43 (s, 3H), 2.34-2.23 (m, 2H), 2.04-1.85 (m, 2H).

### Example 176: N-(5-((2-(2-oxa-5-azaspiro[3.4]octan-5-yl)ethyl)carbamoyl)-2-methylpyridin-3-yl)-2-(1,3-dimethyl-1H-pyrazol-4-yl)pyrazolo[5,1-b]thiazole-7-carboxamide

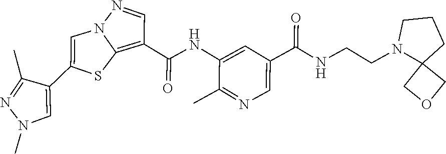 [SMILES: Cc1ncc(C(=O)NCCN2CCCC23COC3)cc1NC(=O)c1cnn2cc(-c3cn(C)nc3C)sc12]

To the mixture of 2-(2-oxa-5-azaspiro[3.4]octan-5-yl) ethanamine (100 mg, 0.640 mmol) in DMF (3 mL) was added 5-(2-(1,3-dimethyl-1H-pyrazol-4-yl)pyrazolo[5,1-b]thiazole-7-carboxamido)-6-methylnicotinic acid (230 mg, 0.580 mmol) and DIEA (391 μL, 2.366 mmol), then HATU(266 mg, 0.700 mmol) was added. The resulting mixture was stirred at room temperature for 2 h. The reaction mixture was concentrated under vacuum to give product as brown solid. The mixture purified by column chromatography over 4 g silica gel (eluent: DCM/MeOH=100/0-80/20), and the fractions were collected and monitored by LCMS. The mixture was concentrated under vacuum to give crude product, which was purified by preparative high-performance liquid chromatography over column: Phenomenex C18 80*40 mm*3 um to give the final compound N-(5-((2-(2-oxa-5-azaspiro[3.4]octan-5-yl)ethyl)carbamoyl)-2-methylpyridin-3-yl)-2-(1,3-dimethyl-1H-pyrazol-4-yl)pyrazolo[5,1-b]thiazole-7-carboxamide (40 mg, 35%) as a white solid. LCMS (ESI): mass for C₂₆H₃₀N₈O₃S: 534.6; m/z found: 535.2 [M+H]⁺. ¹H NMR (400 MHz, CD₃OD) δ 8.85-8.69 (m, 1H), 8.50-8.39 (m, 1H), 8.37-8.28 (m, 1H), 8.19-8.08 (m, 1H), 8.00-7.77 (m, 1H), 4.85-4.82 (m, 2H), 4.57-4.45 (m, 2H), 3.96-3.82 (m, 3H), 3.69-3.50 (m, 2H), 3.21-3.10 (m, 2H), 2.98-2.83 (m, 2H), 2.71-2.55 (m, 3H), 2.49-2.37 (m, 3H), 2.26-2.12 (m, 2H), 1.88-1.72 (m, 2H).

### Example 177: N-(5-((2-(5-azaspiro[2.4]heptan-5-yl)ethyl)carbamoyl)-2-methylpyridin-3-yl)-2-(1,3-dimethyl-1H-pyrazol-4-yl)pyrazolo[5,1-b]thiazole-7-carboxamide

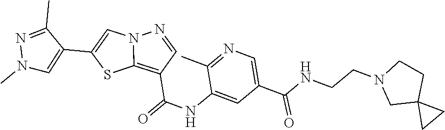 [SMILES: Cc1ncc(C(=O)NCCN2CCC3(CC3)C2)cc1NC(=O)c1cnn2cc(-c3cn(C)nc3C)sc12]

To a solution of 5-(2-(1,3-dimethyl-1H-pyrazol-4-yl)pyrazolo[5,1-b]thiazole-7-carboxamido)-6-methylnicotinic acid (100 mg, 0.25 mmol), HATU (144 mg, 0.38 mmol) and N,N-diisopropylethylamine (98 mg, 0.76 mmol) in N,N-dimethylformamide (7 mL) was added 2-(5-azaspiro[2.4]heptan-5-yl)ethanamine (40 mg, 0.29 mmol). The mixture stirred at 25° C. for 2 hours and then concentrated under vacuum to give the crude product, which was purified by preparative high-performance liquid chromatography over column: Welch Xtimate C18 150*30 mm*5 um to give the title compound N-(5-((2-(5-azaspiro[2.4]heptan-5-yl)ethyl)carbamoyl)-2-methylpyridin-3-yl)-2-(1,3-dimethyl-1H-pyrazol-4-yl)pyrazolo[5,1-b]thiazole-7-carboxamide (38 mg, FA, 27%) as a white solid. LCMS (ESI): mass calcd. for C₂₆H₃₀N₈O₂S, 518.634 m/z found, 519.3 [M+H]⁺. ¹H NMR (400 MHz, METHANOL-d₄) δ 8.82 (s, 1H), 8.53 (br s, 1H), 8.45 (s, 1H), 8.37 (s, 1H), 8.13 (s, 1H), 7.94 (s, 1H), 3.89 (s, 3H), 3.73 (t, J=6.0 Hz, 2H), 3.42 (br t, J=6.9 Hz, 2H), 3.27 (br t, J=5.6 Hz, 2H), 3.18 (s, 2H), 2.64 (s, 3H), 2.43 (s, 3H), 2.04 (t, J=7.3 Hz, 2H), 0.78-0.71 (m, 4H).

### Example 178: N-(5-((2-(2-azabicyclo[2.2.2]octan-2-yl)ethyl)carbamoyl)-2-methylpyridin-3-yl)-2-(1,3-dimethyl-1H-pyrazol-4-yl)pyrazolo[5,1-b]thiazole-7-carboxamide

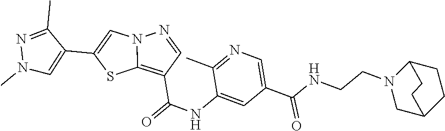 [SMILES: Cc1ncc(C(=O)NCCN2CC3CCC2CC3)cc1NC(=O)c1cnn2cc(-c3cn(C)nc3C)sc12]

To a solution of 5-(2-(1,3-dimethyl-1H-pyrazol-4-yl)pyrazolo[5,1-b]thiazole-7-carboxamido)-6-methylnicotinic acid (100 mg, 0.25 mmol), HATU (144 mg, 0.38 mmol) and N,N-diisopropylethylamine (98 mg, 0.76 mmol) in N,N-dimethylformamide (7 mL) was added 2-(2-azabicyclo[2.2.2]octan-2-yl)ethanamine (44 mg, 0.29 mmol). The mixture stirred at 25° C. for 2 hours and then concentrated under vacuum to give the crude product, which was purified by preparative high-performance liquid chromatography over column: Welch Xtimate C18 150*30 mm*5 um to give the title compound N-(5-((2-(2-azabicyclo[2.2.2]octan-2-yl)ethyl)carbamoyl)-2-methylpyridin-3-yl)-2-(1,3-dimethyl-1H-pyrazol-4-yl)pyrazolo[5,1-b]thiazole-7-carboxamide (44 mg, Formic acid salt, 30%) as a white solid. LCMS (ESI): mass calcd. for C₂₇H₃₂N₈O₂S, 532.66 m/z found, 533.3 [M+H]⁺. ¹H NMR (400 MHz, METHANOL-d₄) δ 8.80 (s, 1H), 8.53 (s, 1H), 8.42 (s, 1H), 8.36 (s, 1H), 8.10 (d, J=3.5 Hz, 1H), 7.91 (s, 1H), 3.87 (s, 3H), 3.74 (t, J=6.0 Hz, 2H), 3.51 (br s, 1H), 3.40 (t, J=6.1 Hz, 2H), 3.35 (br s, 1H), 2.62 (s, 3H), 2.41 (s, 3H), 2.16 (br s, 2H), 1.97 (br s, 1H), 1.93-1.66 (m, 7H).

### Example 179: N-(5-((2-(2-oxa-5-azaspiro[3.4]octan-5-yl)ethyl)carbamoyl)-2-methylpyridin-3-yl)-2-(1,3,5-trimethyl-1H-pyrazol-4-yl)pyrazolo[5,1-b]thiazole-7-carboxamide

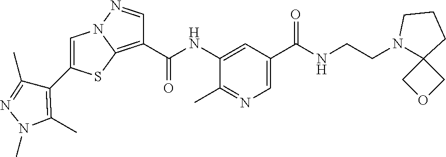 [SMILES: Cc1ncc(C(=O)NCCN2CCCC23COC3)cc1NC(=O)c1cnn2cc(-c3c(C)nn(C)c3C)sc12]

### Step a: methyl 6-methyl-5-(2-(1,3,5-trimethyl-1H-pyrazol-4-yl)pyrazolo[5,1-b]thiazole-7-carboxamido) nicotinate

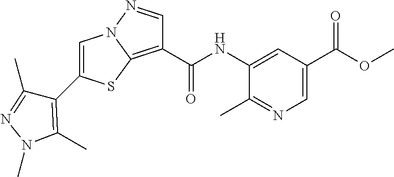 [SMILES: COC(=O)c1cnc(C)c(NC(=O)c2cnn3cc(-c4c(C)nn(C)c4C)sc23)c1]

To a solution of methyl 5-(2-bromopyrazolo[5,1-b]thiazole-7-carboxamido)-6-methylnicotinate (2.0 g, 5.060 mmol) in mixture of dioxane/H₂O (4:1, 125 mL) was added (1,3,5-trimethyl-1H-pyrazol-4-yl)boronic acid (1.92 g, 12.47 mmol), postassium carbonate (4.6 g, 33.28 mmol) and [1,1′-bis(diphenylphosphino)freeocene]dichloropalladium(II) (4.6 g, 33.28 mmol) under N₂. The resulting mixture was heated at 90° C. and stirred for 12 hours. LCMS showed most of the starting material was consumed and the desired mass was detected. The mixture was concentrated under vacuum to afford methyl 6-methyl-5-(2-(1,3,5-trimethyl-1H-pyrazol-4-yl)pyrazolo[5,1-b]thiazole-7-carboxamido) nicotinate (2.15 g, 10%) as a black solid. LCMS (ESI): mass for C₂₀H₂₀N₆O₃S: 424.5; m/z found: 425.0 [M+H]⁺.

### Step b: 6-methyl-5-(2-(1,3,5-trimethyl-1H-pyrazol-4-yl)pyrazolo[5,1-b]thiazole-7-carboxamido)nicotinic acid

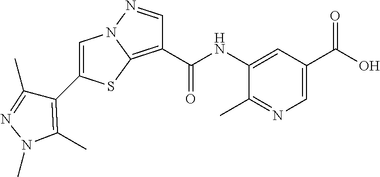 [SMILES: Cc1ncc(C(=O)O)cc1NC(=O)c1cnn2cc(-c3c(C)nn(C)c3C)sc12]

To a solution of methyl 6-methyl-5-((1-methyl-6-((2-methylpyridin-3-yl)amino)-1H-pyrazolo[3,4-d]pyrimidin-3-yl)amino) nicotinate (2.15 g, 0.525 mmol) in mixture of Methanol:THF:H₂O (1:1:1, 90 mL) was added LiOH (40 mg, 1.670 mmol). The mixture was stirred at room temperature for 1.5 h. LCMS showed the reaction was completed. The resultant was evaporated under vacuum, then adjusted to pH=3-4 with 1 mol/L HCl. The mixture was filtered under reduced pressure and the pad was washed with water (5 mL×3). The filter cake was triturated with ethyl acetate and MeOH, then filtered under reduced pressure to give the product as a brown solid. But LCMS showed the compound contained a lot of impurities, which was purified by preparative high-performance liquid chromatography over column: Phenomenex C18 80*40 mm*3 um to give the final compound. The fractions were collected and the solvent was removed to give 6-methyl-5-(2-(1,3,5-trimethyl-1H-pyrazol-4-yl)pyrazolo[5,1-b]thiazole-7-carboxamido) nicotinic acid (260 mg, 15%) as a brown solid.

### Step c: N-(5-((2-(2-oxa-5-azaspiro[3.4]octan-5-yl)ethyl)carbamoyl)-2-methylpyridin-3-yl)-2-(1,3,5-trimethyl-1H-pyrazol-4-yl)pyrazolo[5,1-b]thiazole-7-carboxamide

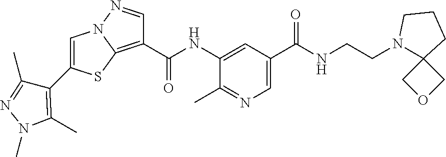 [SMILES: Cc1ncc(C(=O)NCCN2CCCC23COC3)cc1NC(=O)c1cnn2cc(-c3c(C)nn(C)c3C)sc12]

To a solution of 2-(2-oxa-5-azaspiro[3.4]octan-5-yl) ethanamine (37 mg, 0.237 mmol) in DMF (3 mL) was added 6-methyl-5-(2-(1,3,5-trimethyl-1H-pyrazol-4-yl)pyrazolo[5,1-b]thiazole-7-carboxamido)nicotinic acid (100 mg, 0.181 mmol) and DIEA (122 μL, 0.738 mmol), then HATU (83 mg, 0.218 mmol) was added. The resulting mixture was stirred at room temperature for 2 h. The reaction mixture was concentrated under vacuum to give product as brown solid. The mixture was purified by column chromatography over 4 g silica gel (eluent: DCM/MeOH=100/0-80/20), and the fractions were collected and monitored by LCMS. The fractions were collected, and the solvent was removed to give a brown solid, which was purified by preparative high-performance liquid chromatography over column: Phenomenex C18 80*40 mm*3 um to give the final compound. The fractions were collected and the solvent was removed to give N-(5-((2-(2-oxa-5-azaspiro[3.4]octan-5-yl)ethyl)carbamoyl)-2-methylpyridin-3-yl)-2-(1,3,5-trimethyl-1H-pyrazol-4-yl)pyrazolo[5,1-b]thiazole-7-carboxamide (26.2 mg, 30%) as a white solid. LCMS (ESI): mass for C₂₆H₃₀N₈O₃S: 548.7; m/z found: 549.3 [M+H]⁺. ¹H NMR (400 MHz, CD₃OD) δ 8.81-8.78 (m, 1H), 8.48-8.45 (m, 1H), 8.34-8.31 (m, 1H), 8.06-8.04 (m, 1H), 4.83 (d, J=6.8 Hz, 2H), 4.57-4.48 (m, 2H), 3.87-3.76 (m, 3H), 3.70-3.58 (m, 2H), 3.23-3.11 (m, 2H), 2.95-2.85 (m, 2H), 2.68-2.59 (m, 3H), 2.46-2.38 (m, 3H), 2.36-2.29 (m, 3H), 2.25-2.14 (m, 2H), 1.89-1.74 (m, 2H).

### Example 180: N-(5-((2-(2-azabicyclo[2.2.2]octan-2-yl)ethyl)carbamoyl)-2-methylpyridin-3-yl)-2-(1-(difluoromethyl)-1H-pyrazol-4-yl)pyrazolo[5,1-b]thiazole-7-carboxamide

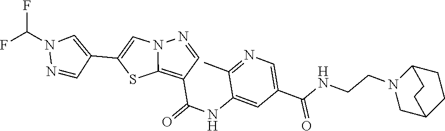 [SMILES: Cc1ncc(C(=O)NCCN2CC3CCC2CC3)cc1NC(=O)c1cnn2cc(-c3cnn(C(F)F)c3)sc12]

To a solution of N-(5-(3-(2-azabicyclo[2.2.2]octan-2-yl)propanamido)-2-methylpyridin-3-yl)-2-bromopyrazolo[5,1-b]thiazole-7-carboxamide (200 mg, 0.36 mmol) and 1-(difluoromethyl)-4-(4,4,5,5-tetramethyl-1,3,2-dioxaborolan-2-yl)-1H-pyrazole (106 mg, 0.44 mmol) in 1,4-dioxane (16 mL) and H₂O (4 mL) was added [1,1-Bis(diphenylphosphino)ferrocene]palladium(II) chloride dichloromethane complex (89 mg, 0.11 mmol) and Cs₂CO₃ (355 mg, 1.1 mmol) under N2. The resulting mixture was stirred at 90° C. under N₂ for 12 h before cooled to 25° C. The reaction mixture was diluted with ethyl acetate (30 mL) and filtered over celite. The filtrate was concentrated to the crude product, which was treated with ethyl acetate (90 mL) and water (30 mL). The organic phase was washed with 3 M HCl aqueous solution, dried over anhydrous Na₂SO₄ and filtered. The filtrate was concentrated under reduced pressure to give the crude product, which was purified by preparative high-performance liquid chromatography over column: Phenomenex Gemini-NX C18 75*30 mm*3 um, then SFC over column: DAICEL CHIRALCEL OD(250 mm*30 mm, 10 um) to give the title compound N-(5-((2-(2-azabicyclo[2.2.2]octan-2-yl)ethyl)carbamoyl)-2-methylpyridin-3-yl)-2-(1-(difluoromethyl)-1H-pyrazol-4-yl)pyrazolo[5,1-b]thiazole-7-carboxamide (25 mg, 61%) as a white solid. LCMS (ESI): mass calcd. for C₂₆H₂₈F₂N₈O₂S, 554.615; m/z found, 555.3 [M+H]⁺. ¹H NMR (400 MHz, METHANOL-d₄) δ 8.82 (d, J=2.03 Hz, 1H), 8.57 (s, 1H), 8.45 (d, J=11.09 Hz, 2H), 8.36 (d, J=1.91 Hz, 1H), 8.13 (s, 1H), 7.39-7.75 (m, 1H), 3.68 (br, t, J=6.38 Hz, 2H), 3.16 (br, s, 5H), 2.64 (s, 3H), 2.11 (br, s, 2H), 1.87 (br, s, 1H), 1.69-1.82 (m, 6H).

### Example 181: N-(5-((2-(2-azabicyclo[2.2.2]octan-2-yl)ethyl)carbamoyl)-2-methylpyridin-3-yl)-2-(1-cyclopropyl-1H-pyrazol-4-yl)pyrazolo[5,1-b]thiazole-7-carboxamide

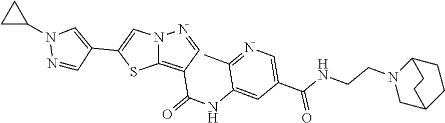 [SMILES: Cc1ncc(C(=O)NCCN2CC3CCC2CC3)cc1NC(=O)c1cnn2cc(-c3cnn(C4CC4)c3)sc12]

To a solution of N-(5-(3-(2-azabicyclo[2.2.2]octan-2-yl)propanamido)-2-methylpyridin-3-yl)-2-bromopyrazolo[5,1-b]thiazole-7-carboxamide (200 mg, 0.36 mmol) and 2-cyclopropyl-4,4,5,5-tetramethyl-1,3,2-dioxaborolane (73 mg, 0.44 mmol) in 1,4-dioxane (16 mL) and H₂O (4 mL) was added [1,1-bis(diphenylphosphino)ferrocene]palladium(II) chloride dichloromethane complex (89 mg, 0.11 mmol) and Cs₂CO₃ (355 mg, 1.1 mmol) under N₂. The resulting mixture was stirred at 90° C. under N₂ for 12 h before cooled to 25° C. The reaction mixture was diluted with ethyl acetate (30 mL) and filtered over celite. The filtrate was concentrated to the crude product, which was treated with ethyl acetate (90 mL) and water (30 mL). The organic phase was washed with 3 M HCl aqueous solution, dried over anhydrous Na₂SO₄ and filtered. The filtrate was concentrated under reduced pressure to give the crude product, which was purified by preparative high-performance liquid chromatography over column: Phenomenex Gemini-NX C18 75*30 mm*3 um, then SFC over column: DAICEL CHIRALCEL OD(250 mm*30 mm, 10 um) to give the title compound N-(5-((2-(2-azabicyclo[2.2.2]octan-2-yl)ethyl)carbamoyl)-2-methylpyridin-3-yl)-2-(1-cyclopropyl-1H-pyrazol-4-yl)pyrazolo[5,1-b]thiazole-7-carboxamide (43 mg, 59%) as a white solid. LCMS (ESI): mass calcd. for C₂₈H₃₂N₈O₂S, 544.671; m/z found, 545.3 [M+H]⁺. ¹H NMR (400 MHz, METHANOL-d₄) δ 8.67 (d, J=1.91 Hz, 1H), 8.30 (s, 1H), 8.21 (d, J=1.91 Hz, 1H), 8.13 (d, J=1.07 Hz, 1H), 8.02 (s, 1H), 7.71 (s, 1H), 3.61 (tt, J=7.27, 3.81 Hz, 1H), 3.44 (t, J=7.03 Hz, 2H), 2.66-2.76 (m, 4H), 2.57 (br s, 1H), 2.50 (s, 3H), 1.86-1.98 (m, 2H), 1.54-1.62 (m, 3H), 1.42-1.53 (m, 4H), 0.94-1.08 (m, 4H).

### Example 182: N-(5-((2-(2,2-dimethylpyrrolidin-1-yl)ethyl)carbamoyl)-3-methylthiophen-2-yl)-2-(1-methyl-1H-pyrazol-4-yl)pyrazolo[5,1-b]thiazole-7-carboxamide

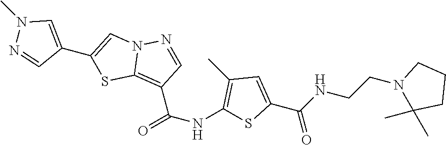 [SMILES: Cc1cc(C(=O)NCCN2CCCC2(C)C)sc1NC(=O)c1cnn2cc(-c3cnn(C)c3)sc12]

To a solution of 4-methyl-5-(2-(1-methyl-1H-pyrazol-4-yl)pyrazolo[5,1-b]thiazole-7-carboxamido)thiophene-2-carboxylic acid (100 mg, 0.26 mmol), 2-(2,2-dimethylpyrrolidin-1-yl)ethanamine (37 mg, 0.26 mmol) and N-ethyl-N-isopropylpropan-2-amine (0.18 mL, 1.0 mmol) in DMF (4 mL) was added HATU (196 mg, 0.52 mmol). The resulting mixture was stirred at 25° C. for 16 hours and then concentrated under vacuum to give the crude product, which was purified by preparative high-performance liquid chromatography over column: Boston Prime C18 150*30 mm*5 um to give the title compound N-(5-((2-(2,2-dimethylpyrrolidin-1-yl)ethyl)carbamoyl)-3-methylthiophen-2-yl)-2-(1-methyl-1H-pyrazol-4-yl)pyrazolo[5,1-b]thiazole-7-carboxamide (30 mg, 26%) as a white solid. LCMS (ESI): mass calcd. for C₂₄H₂₉N₇O₂S₂, 511.7; m/z found, 512.2 [M+H]⁺. ¹H NMR (400 MHz, METHANOL-d₄) δ 8.45 (br s, 1H), 8.21 (s, 1H), 8.03 (s, 1H), 7.83 (s, 1H), 7.38 (s, 1H), 3.95 (s, 3H), 3.47 (t, J=7.03 Hz, 2H), 2.92 (br t, J=7.28 Hz, 2H), 2.67 (br d, J=5.27 Hz, 2H), 2.29 (s, 3H), 1.79-1.87 (m, 2H), 1.67-1.74 (m, 2H), 1.07 (s, 6H).

### Example 183: N-(5-((2-(4,4-dimethylpiperidin-1-yl)ethyl)carbamoyl)-3-methylthiophen-2-yl)-2-(1-methyl-1H-pyrazol-4-yl)pyrazolo[5,1-b]thiazole-7-carboxamide

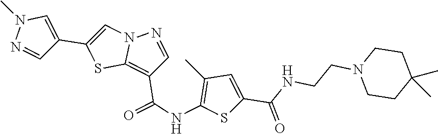

To a solution of 4-methyl-5-(2-(1-methyl-1H-pyrazol-4-yl)pyrazolo[5,1-b]thiazole-7-carboxamido)thiophene-2-carboxylic acid (60 mg, 0.16 mmol), 2-(4,4-dimethylpiperidin-1-yl)ethanamine (24 mg, 0.16 mmol) and N-ethyl-N-isopropylpropan-2-amine (0.08 mL, 0.62 mmol) in DMF (4 mL) was added HATU (118 mg, 0.31 mmol). The resulting mixture was stirred at 25° C. for 16 hours and then concentrated under vacuum to give the crude product, which was purified by preparative high-performance liquid chromatography over column: Boston Green ODS 150*30 mm*5 um to give the title compound N-(5-((2-(4,4-dimethylpiperidin-1-yl)ethyl)carbamoyl)-3-methylthiophen-2-yl)-2-(1-methyl-1H-pyrazol-4-yl)pyrazolo[5,1-b]thiazole-7-carboxamide (20 mg, 23%) as a white solid. LCMS (ESI): mass calcd. for C₂₅H₃₁N₇O₂S₂, 525.7; m/z found, 526.2 [M+H]⁺. ¹H NMR (400 MHz, METHANOL-d₄) δ 8.46-8.50 (m, 0.458H), 8.44 (br s, 1H), 8.43-8.46 (m, 1H), 8.21 (s, 1H), 8.02 (s, 1H), 7.81 (s, 1H), 7.39 (s, 1H), 3.93 (s, 3H), 3.66 (br t, J=5.95 Hz, 2H), 3.31-3.35 (m, 1H), 3.33 (s, 1H), 3.19 (br d, J=5.29 Hz, 4H), 2.28 (s, 3H), 1.64 (br t, J=5.73 Hz, 4H), 1.05 (s, 6H).

### Example 184: 2-(6-aminopyridin-3-yl)-N-(5-((2-(2,2-dimethylpyrrolidin-1-yl)ethyl)carbamoyl)-3-methylthiophen-2-yl)pyrazolo[5,1-b]thiazole-7-carboxamide

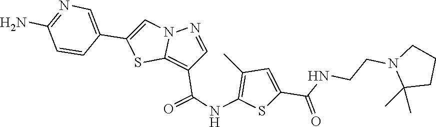 [SMILES: Cc1cc(C(=O)NCCN2CCCC2(C)C)sc1NC(=O)c1cnn2cc(-c3ccc(N)nc3)sc12]

To a solution of methyl 2-bromo-N-(5-((2-(2,2-dimethylpyrrolidin-1-yl)ethyl)carbamoyl)-3-methylthiophen-2-yl)pyrazolo[5,1-b]thiazole-7-carboxamide (70 mg, 0.14 mmol) and 5-(4,4,5,5-tetramethyl-1,3,2-dioxaborolan-2-yl)pyridin-2-amine (45 mg, 0.21 mmol) in 1,4-dioxane (4 mL) and H₂O (1 mL) was added Pd(dppf)Cl₂·CH₂Cl₂ (22 mg, 0.03 mmol) and K₂CO₃ (57 mg, 0.41 mmol) under N₂. The resulting mixture was stirred at 90° C. for 12 h before cooled to 25° C. The reaction mixture was diluted with ethyl acetate (10 mL) and filtered over celite. The filtrate was concentrated to the crude product, which was purified by preparative high-performance liquid chromatography over column: Boston Green ODS 150*30 mm*5 um to give the title compound 2-(6-aminopyridin-3-yl)-N-(5-((2-(2,2-dimethylpyrrolidin-1-yl)ethyl)carbamoyl)-3-methylthiophen-2-yl)pyrazolo[5,1-b]thiazole-7-carboxamide (18 mg, 22%) as a yellow solid. LCMS (ESI): mass calcd. for C₂₅H₂₉N₇O₂S₂, 523.7; m/z found, 524.1 [M+H]⁺. ¹H NMR (400 MHz, METHANOL-d₄) δ 8.48 (br s, 1H), 8.33 (s, 1H), 8.22 (d, J=2.43 Hz, 1H), 7.77 (dd, J=8.71, 2.32 Hz, 1H), 8.44 (br s, 1H), 7.45 (s, 1H), 6.68 (d, J=8.82 Hz, 1H), 3.71 (br s, 2H), 3.32-3.35 (m, 4H), 2.31 (s, 3H), 2.13 (br d, J=7.28 Hz, 2H), 2.06 (br d, J=7.06 Hz, 2H), 1.40 (br s, 6H).

### Example 185: N-(5-((2-(2,2-dimethylpyrrolidin-1-yl)ethyl)carbamoyl)-3-methylthiophen-2-yl)-2-(pyrimidin-5-yl)pyrazolo[5,1-b]thiazole-7-carboxamide

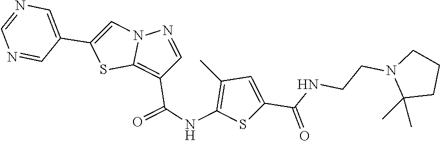 [SMILES: Cc1cc(C(=O)NCCN2CCCC2(C)C)sc1NC(=O)c1cnn2cc(-c3cncnc3)sc12]

To a solution of methyl 2-bromo-N-(5-((2-(2,2-dimethylpyrrolidin-1-yl)ethyl)carbamoyl)-3-methylthiophen-2-yl)pyrazolo[5,1-b]thiazole-7-carboxamide (120 mg, 0.24 mmol) and pyrimidin-5-ylboronic acid (73 mg, 0.35 mmol) in 1,4-dioxane (4 mL) and H₂O (1 mL) was added Pd(dppf)Cl₂·CH₂Cl₂ (19 mg, 0.02 mmol) and NaHCO₃ (59 mg, 0.71 mmol) under N₂. The resulting mixture was stirred at 95° C. for 16 h before cooled to 25° C. The reaction mixture was diluted with ethyl acetate (10 mL) and filtered over celite. The filtrate was concentrated to the crude product, which was purified by preparative high-performance liquid chromatography over column: Boston Green ODS 150*30 mm*5 um to give the title compound N-(5-((2-(2,2-dimethylpyrrolidin-1-yl)ethyl)carbamoyl)-3-methylthiophen-2-yl)-2-(pyrimidin-5-yl)pyrazolo[5,1-b]thiazole-7-carboxamide (13 mg, 11%) as a yellow solid. LCMS (ESI): mass calcd. for C₂₄H₂₇N₇O₂S₂, 509.6; m/z found, 510.2 [M+H]⁺. ¹H NMR (400 MHz, METHANOL-d₄) δ 9.10-9.18 (m, 3H), 8.77 (s, 1H), 8.55 (br s, 1H), 7.40 (s, 1H), 3.63 (br s, 2H), 3.40 (br s, 2H), 3.11 (br s, 2H), 2.29 (s, 3H), 2.06 (br s, 2H), 1.95 (br s, 2H), 1.30 (br s, 6H).

### Example 186: N-(5-((2-(2,2-dimethylpyrrolidin-1-yl)ethyl)carbamoyl)-3-methylthiophen-2-yl)-2-(2-methyl-2H-1,2,3-triazol-4-yl)pyrazolo[5,1-b]thiazole-7-carboxamide

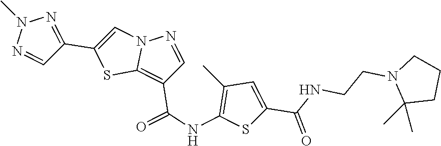 [SMILES: Cc1cc(C(=O)NCCN2CCCC2(C)C)sc1NC(=O)c1cnn2cc(-c3cnn(C)n3)sc12]

To a solution of 4-bromo-2-methyl-2H-1,2,3-triazole (200 mg, 1.2 mmol), 4,4,4′,4′,5,5,5′,5′-octamethyl-2,2′-bi(1,3,2-dioxaborolane) (627 mg, 2.5 mmol) and potassium acetate (364 mg, 3.7 mmol) in dioxane (6 mL) was added Pd(dppf)Cl₂·CH₂Cl₂ (101 mg, 0.12 mmol) at room temperature. The resulting mixture was stirred at 80° C. overnight before cooling to room temperature. Then methyl 2-bromo-N-(5-((2-(2,2-dimethylpyrrolidin-1-yl)ethyl)carbamoyl)-3-methylthiophen-2-yl)pyrazolo[5,1-b]thiazole-7-carboxamide (315 mg, 0.62 mmol), K₃PO₄ (786 mg, 3.7 mmol), water (1.5 mL) and Pd(dppf)Cl₂·CH₂Cl₂ (101 mg, 0.12 mmol) was added. The reaction mixture was stirred at 95° C. overnight before cooling to room temperature. The reaction mixture was filtered through a pad of celite and the filtrate was concentrated under reduced pressure to give crude product, which was purified by preparative high-performance liquid chromatography over column: Boston Green ODS 150*30 mm*5 um to give the title compound N-(5-((2-(2,2-dimethylpyrrolidin-1-yl)ethyl)carbamoyl)-3-methylthiophen-2-yl)-2-(2-methyl-2H-1,2,3-triazol-4-yl)pyrazolo[5,1-b]thiazole-7-carboxamide (17 mg, 2%) as a yellow solid. LCMS (ESI): mass calcd. for C₂₃H₂₈N₈O₂S₂, 512.7; m/z found, 513.2 [M+H]⁺. ¹H NMR (400 MHz, DMSO-d₆) 10.33 (br s, 1H), 8.87 (s, 1H), 8.55 (s, 0.871H), 8.17 (s, 2H), 8.11 (br s, 1H), 7.31 (s, 1H), 4.12 (s, 3 H), 3.15 (br d, J=6.17 Hz, 2H), 2.88-3.22 (m, 2H), 2.66 (br t, J=7.06 Hz, 2H), 2.13 (s, 3H), 1.57 (br d, J=7.50 Hz, 2H), 1.42-1.49 (m, 2H), 0.83 (s, 6H).

### Example 187: N-(5-((2-(2,2-dimethylpyrrolidin-1-yl)ethyl)carbamoyl)-3-methylthiophen-2-yl)-2-(pyridin-4-yl)pyrazolo[5,1-b]thiazole-7-carboxamide

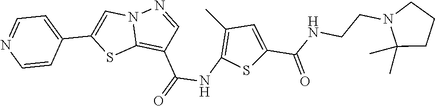 [SMILES: Cc1cc(C(=O)NCCN2CCCC2(C)C)sc1NC(=O)c1cnn2cc(-c3ccncc3)sc12]

To a solution of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (150 mg, 0.30 mmol) and 4-(4,4,5,5-tetramethyl-1,3,2-dioxaborolan-2-yl)pyridine (72.3 mg, 0.35 mmol) in 1,4-dioxane (4 mL) and H₂O (1 mL) was added [1,1-Bis(diphenylphosphino)ferrocene]palladium(II) chloride dichloromethane complex (24.0 mg, 0.03 mmol) and K₃PO₄ (187 mg, 0.88 mmol) under N₂. The resulting mixture was stirred at 100° C. for 12 h before cooled to 25° C. The reaction mixture was diluted with ethyl acetate (10 mL) and filtered over celite. The filtrate was concentrated to the crude product, which was treated with dichloromethane (10 mL) and water (10 mL). The organic phase was concentrated under reduced pressure to give the crude product, which was purified by preparative high-performance liquid chromatography over column: Boston Prime C18 150*30 mm*5 um to give the title compound N-(5-((2-(2,2-dimethylpyrrolidin-1-yl)ethyl)carbamoyl)-3-methylthiophen-2-yl)-2-(pyridin-4-yl)pyrazolo[5,1-b]thiazole-7-carboxamide (14 mg, 9%) as a yellow oil. LCMS (ESI): mass calcd. for C₂₅H₂₈N₆O₂S₂, 508.659; m/z found, 509.2 [M+H]⁺. ¹H NMR (400 MHz, METHANOL-d₄) δ 8.89 (s, 1H), 8.66-8.62 (m, 2H), 8.57 (s, 1H), 8.54 (s, 0.56H), 7.79-7.75 (m, 2H), 7.42 (s, 1H), 3.61 (br s, 2H), 3.29-3.19 (m, 2H), 3.04 (br s, 2H), 2.31 (s, 3H), 2.07-1.88 (m, 4H), 1.27 (br s, 6H).

### Example 188: N-(5-((2-(2,2-dimethylpyrrolidin-1-yl)ethyl)carbamoyl)-3-methylthiophen-2-yl)-2-(1-methyl-1H-pyrazol-3-yl)pyrazolo[5,1-b]thiazole-7-carboxamide

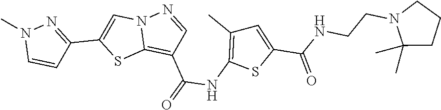 [SMILES: Cc1cc(C(=O)NCCN2CCCC2(C)C)sc1NC(=O)c1cnn2cc(-c3ccn(C)n3)sc12]

To a solution of 2-bromo-N-(5-((2-(2,2-dimethylpyrrolidin-1-yl)ethyl)carbamoyl)-3-methylthiophen-2-yl)pyrazolo[5,1-b]thiazole-7-carboxamide (220 mg, 0.20 mmol) and 1-methyl-4-(4,4,5,5-tetramethyl-1,3,2-dioxaborolan-2-yl)-1H-pyrazole (62.1 mg, 0.30 mmol) in 1,4-dioxane (3 mL) and H₂O (0.75 mL) was added [1,1-bis(diphenylphosphino)ferrocene]palladium(II) chloride dichloromethane complex (16.2 mg, 0.02 mmol) and K₃PO₄ (127 mg, 0.60 mmol) under N₂. The resulting mixture was stirred at 95° C. for 12 h before cooled to 25° C. The reaction mixture was diluted with ethyl acetate (10 mL) and filtered over celite. The filtrate was concentrated to the crude product, which was treated with dichloromethane (10 mL) and water (10 mL). The organic phase was concentrated under reduced pressure to give the crude product, which was purified by preparative high-performance liquid chromatography over column: Boston Prime C18 150*30 mm*5 um to give the title compound N-(5-((2-(2,2-dimethylpyrrolidin-1-yl)ethyl)carbamoyl)-3-methylthiophen-2-yl)-2-(1-methyl-1H-pyrazol-3-yl)pyrazolo[5,1-b]thiazole-7-carboxamide (23.2 mg, 22%) as a yellow oil. LCMS (ESI): mass calcd. for C₂₄H₂₉N₇O₂S₂, 511.663; m/z found, 512.3 [M+H]⁺. ¹H NMR (400 MHz, METHANOL-d₄) δ 8.48 (s, 1H), 8.26 (s, 1H), 8.07 (s, 1H), 7.86 (s, 1H), 7.43 (s, 1H), 3.97 (s, 3H), 3.64-3.49 (m, 2H), 3.30-3.19 (m, 2H), 2.98 (br s, 2H), 2.32 (s, 3H), 2.06-1.83 (m, 4H), 1.31-1.16 (m, 6H).

### Example 189: N-(5-((2-(3,3-dimethylpyrrolidin-1-yl)ethyl)carbamoyl)-3-methylthiophen-2-yl)-2-(1-methyl-1H-pyrazol-4-yl)pyrazolo[5,1-b]thiazole-7-carboxamide

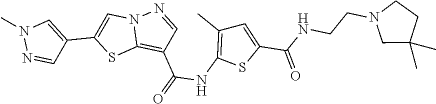

To a solution of methyl 2-bromo-N-(5-((2-(2,2-dimethylpyrrolidin-1-yl)ethyl)carbamoyl)-3-methylthiophen-2-yl)pyrazolo[5,1-b]thiazole-7-carboxamide (200 mg, 0.39 mmol) and 1-methyl-4-(4,4,5,5-tetramethyl-1,3,2-dioxaborolan-2-yl)-1H-pyrazole (98 mg, 0.47 mmol) in 1,4-dioxane (4 mL) and H₂O (1 mL) was added Pd(dppf)Cl₂·CH₂Cl₂ (32 mg, 0.04 mmol) and K₂CO₃ (162 mg, 1.2 mmol) under N₂. The resulting mixture was stirred at 90° C. for 16 h before cooled to 25° C. The reaction mixture was diluted with ethyl acetate (10 mL) and filtered over celite. The filtrate was concentrated to the crude product, which was purified by preparative high-performance liquid chromatography over column: Phenomenex Gemini-NX C18 75*30 mm*3 um to give the title compound N-(5-((2-(3,3-dimethylpyrrolidin-1-yl)ethyl)carbamoyl)-3-methylthiophen-2-yl)-2-(1-methyl-1H-pyrazol-4-yl)pyrazolo[5,1-b]thiazole-7-carboxamide (13 mg, 11%) as a yellow solid. LCMS (ESI): mass calcd. for C₂₄H₂₉N₇O₂S₂, 511.7; m/z found, 512.2 [M+H]⁺. ¹H NMR (400 MHz, METHANOL-d₄) 8.44 (s, 1H), 8.21 (s, 1H), 8.02 (s, 1H), 7.81 (s, 1H), 7.39 (s, 1H), 3.93 (s, 3H), 3.54-3.58 (m, 2H), 3.08 (br s, 2H), 2.81-2.97 (m, 2H), 2.28 (s, 3H), 1.80 (br t, J=6.95 Hz, 2H), 1.28 (br d, J=7.50 Hz, 2H), 1.16 (s, 6H).

### Example 190: N-(5-((2-(cyclopentyl(methyl)amino)ethyl)carbamoyl)-3-methylthiophen-2-yl)-2-(1-methyl-1H-pyrazol-4-yl)pyrazolo[5,1-b]thiazole-7-carboxamide

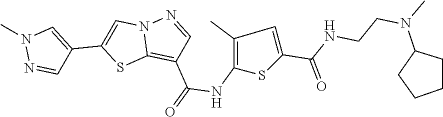 [SMILES: Cc1cc(C(=O)NCCN(C)C2CCCC2)sc1NC(=O)c1cnn2cc(-c3cnn(C)c3)sc12]

### Step a: 2-bromo-N-(5-((2-(cyclopentyl(methyl)amino)ethyl)carbamoyl)-3-methylthiophen-2-yl)pyrazolo[5,1-b]thiazole-7-carboxamide

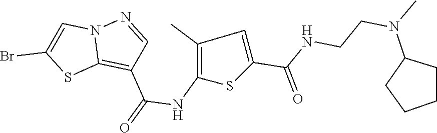 [SMILES: Cc1cc(C(=O)NCCN(C)C2CCCC2)sc1NC(=O)c1cnn2cc(Br)sc12]

To a solution of 5-(2-bromopyrazolo[5,1-b]thiazole-7-carboxamido)-4-methylthiophene-2-carboxylic acid (462 mg, 1.2 mmol), DIEA (0.83 mL, 4.8 mmol) in DMF (5 mL) was added N¹-cyclopentyl-N¹-methylethane-1,2-diamine (170 mg, 1.2 mmol). The mixture was stirred at room temperature for 10 min. Then HATU (909 mg, 2.4 mmol) was added. The mixture was stirred at room temperature overnight. The reaction mixture was concentrated in vacuo to give the crude product. The crude product was purified by column chromatography over silica gel (eluent: DCM/MeOH=100/0-80/20). The desired fractions were evaporated in vacuum to give the title compound 2-bromo-N-(5-((2-(cyclopentyl(methyl)amino)ethyl)carbamoyl)-3-methylthiophen-2-yl)pyrazolo[5,1-b]thiazole-7-carboxamide (450 mg, 74%) as brown oil. LCMS (ESI): mass calcd. for C₂₀H₂₄BrN₅O₂S₂, 510.5; m/z found, 512.1 [M+H]⁺.

### Step b: N-(5-((2-(cyclopentyl(methyl)amino)ethyl)carbamoyl)-3-methylthiophen-2-yl)-2-(1-methyl-1H-pyrazol-4-yl)pyrazolo[5,1-b]thiazole-7-carboxamide

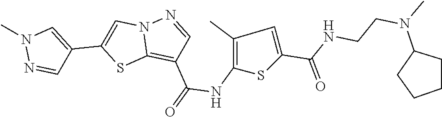 [SMILES: Cc1cc(C(=O)NCCN(C)C2CCCC2)sc1NC(=O)c1cnn2cc(-c3cnn(C)c3)sc12]

To a solution of 2-bromo-N-(5-((2-(cyclopentyl(methyl)amino)ethyl)carbamoyl)-3-methylthiophen-2-yl)pyrazolo[5,1-b]thiazole-7-carboxamide (200 mg, 0.39 mmol), 1-methyl-4-(4,4,5,5-tetramethyl-1,3,2-dioxaborolan-2-yl)-1H-pyrazole (98 mg, 0.47 mmol) and K₂CO₃ (162 mg, 1.2 mmol) in dioxane (4 mL) and H₂O (1 mL) was added Pd(dppf)Cl₂·CH₂Cl₂ (32 mg, 0.04 mmol) at room temperature. The resulting mixture was stirred at 90° C. overnight before cooling to room temperature. The reaction mixture was filtered through a pad of celite and the filtrate was concentrated under reduced pressure to give crude product, which was purified by preparative high-performance liquid chromatography over column: Phenomenex Gemini-NX C18 75*30 mm*3 um to give the title compound N-(5-((2-(cyclopentyl(methyl)amino)ethyl)carbamoyl)-3-methylthiophen-2-yl)-2-(1-methyl-1H-pyrazol-4-yl)pyrazolo[5,1-b]thiazole-7-carboxamide (19 mg, 9%) as a yellow solid. LCMS (ESI): mass calcd. for C₂₄H₂₉N₇O₂S₂, 511.7; m/z found, 512.2 [M+H]⁺. ¹H NMR (400 MHz, METHANOL-d₄) 8.22 (s, 1H) 8.44 (s, 1H), 8.02 (s, 1H), 7.81 (s, 1H), 7.40 (s, 1H), 3.93 (s, 3H), 3.66 (br t, J=6.17 Hz, 2H), 3.52 (br s, 1H), 3.21 (br s, 2H), 2.82 (s, 3H), 2.28 (s, 3H), 2.10 (br s, 2H), 1.79 (br s, 2H), 1.65 (br s, 4H).

### Example 191: N-(5-((2-(4-hydroxypiperidin-1-yl)ethyl)carbamoyl)-3-methylthiophen-2-yl)-2-(1-methyl-1H-pyrazol-4-yl)pyrazolo[5,1-b]thiazole-7-carboxamide

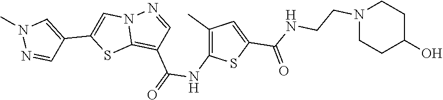 [SMILES: Cc1cc(C(=O)NCCN2CCC(O)CC2)sc1NC(=O)c1cnn2cc(-c3cnn(C)c3)sc12]

To a solution of 4-methyl-5-(2-(1-methyl-1H-pyrazol-4-yl)pyrazolo[5,1-b]thiazole-7-carboxamido)thiophene-2-carboxylic acid (100 mg, 0.26 mmol), 1-(2-aminoethyl)piperidin-4-ol (15 mg, 0.31 mmol) and N-ethyl-N-isopropylpropan-2-amine (133 mg, 1.0 mmol) in DMF (4 mL) was added HATU (196 mg, 0.52 mmol). The resulting mixture was stirred at 25° C. for 16 hours and then concentrated under vacuum to give the crude product, which was purified by preparative high-performance liquid chromatography over column: Boston Prime C18 150*30 mm*5 um to give the title compound N-(5-((2-(4,4-dimethylpiperidin-1-yl)ethyl)carbamoyl)-3-methylthiophen-2-yl)-2-(1-methyl-1H-pyrazol-4-yl)pyrazolo[5,1-b]thiazole-7-carboxamide (46 mg, 34%) as a yellow solid. LCMS (ESI): mass calcd. for C₂₃H₂₇N₇O₃S₂, 513.6; m/z found, 514.2 [M+H]⁺. ¹H NMR (400 MHz, METHANOL-d₄) δ 1.58 (br d, J=10.58 Hz, 2H) 8.43 (br s, 1H), 8.19 (br d, J=3.97 Hz, 1H), 8.02 (br s, 1H), 7.81 (br s, 1H), 7.36 (br s, 1H), 4.59 (br s, 1H), 3.93 (br s, 3H), 3.63 (br s, 1H), 3.49 (br s, 2H), 2.88 (br s, 2H), 2.59 (br s, 2H), 2.26 (br s, 5H), 1.86 (br s, 2H).

### Example 192: 2-(1-methyl-1H-pyrazol-4-yl)-N-(3-methyl-5-((2-(methyl(tetrahydro-2H-pyran-4-yl)amino)ethyl)carbamoyl)thiophen-2-yl)pyrazolo[5,1-b]thiazole-7-carboxamide

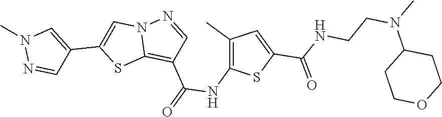 [SMILES: Cc1cc(C(=O)NCCN(C)C2CCOCC2)sc1NC(=O)c1cnn2cc(-c3cnn(C)c3)sc12]

To a solution of 4-methyl-5-(2-(1-methyl-1H-pyrazol-4-yl)pyrazolo[5,1-b]thiazole-7-carboxamido)thiophene-2-carboxylic acid (150 mg, 0.39 mmol), N1-methyl-N1-(tetrahydro-2H-pyran-4-yl)ethane-1,2-diamine (74 mg, 0.47 mmol) and N-ethyl-N-isopropylpropan-2-amine (0.27 mL, 1.5 mmol) in DMF (4 mL) was added HATU (294 mg, 0.77 mmol). The resulting mixture was stirred at 25° C. for 16 hours and then concentrated under vacuum to give the crude product, which was purified by preparative high-performance liquid chromatography over column: Phenomenex Gemini-NX 80*40 mm*3 um to give the title compound 2-(1-methyl-1H-pyrazol-4-yl)-N-(3-methyl-5-((2-(methyl(tetrahydro-2H-pyran-4-yl)amino)ethyl)carbamoyl)thiophen-2-yl)pyrazolo[5,1-b]thiazole-7-carboxamide (36 mg, 17%) as a yellow solid. LCMS (ESI): mass calcd. for C₂₄H₂₉N₇O₃S₂, 527.7; m/z found, 528.2 [M+H]⁺. ¹H NMR (400 MHz, METHANOL-d₄) δ 8.41 (s, 1H), 8.17 (s, 1H), 7.99 (s, 1H), 7.79 (s, 1H), 7.33 (s, 1H), 3.96 (br dd, J=11.14, 3.64 Hz, 2H), 3.91 (s, 3H), 3.36-3.47 (m, 4H), 2.69 (br t, J=6.95 Hz, 2H), 2.60-2.67 (m, 1H), 2.34 (s, 3H), 2.25 (s, 3H), 1.77 (br d, J=12.13 Hz, 2H), 1.48-1.58 (m, 2H).

### Example 193: N-(5-((2-(4-(hydroxymethyl)piperidin-1-yl)ethyl)carbamoyl)-3-methylthiophen-2-yl)-2-(1-methyl-1H-pyrazol-4-yl)pyrazolo[5,1-b]thiazole-7-carboxamide

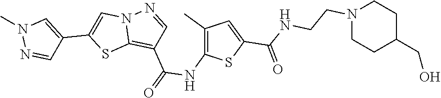 [SMILES: Cc1cc(C(=O)NCCN2CCC(CO)CC2)sc1NC(=O)c1cnn2cc(-c3cnn(C)c3)sc12]

To a solution of 4-methyl-5-(2-(1-methyl-1H-pyrazol-4-yl)pyrazolo[5,1-b]thiazole-7-carboxamido)thiophene-2-carboxylic acid (100 mg, 0.26 mmol), (1-(2-aminoethyl)piperidin-4-yl)methanol (53 mg, 0.31 mmol) and N-ethyl-N-isopropylpropan-2-amine (0.18 mL, 1.0 mmol) in DMF (4 mL) was added HATU (192 mg, 0.56 mmol). The resulting mixture was stirred at 25° C. for 16 hours and then concentrated under vacuum to give the crude product, which was purified by preparative high-performance liquid chromatography over column: Phenomenex Gemini-NX 80*40 mm*3 um to give the title compound N-(5-((2-(4-(hydroxymethyl)piperidin-1-yl)ethyl)carbamoyl)-3-methylthiophen-2-yl)-2-(1-methyl-1H-pyrazol-4-yl)pyrazolo[5,1-b]thiazole-7-carboxamide (40 mg, 27%) as a yellow solid. LCMS (ESI): mass calcd. for C₂₄H₂₉N₇O₃S₂, 527.7; m/z found, 528.2 [M+H]⁺. ¹H NMR (400 MHz, METHANOL-d₄) 8.44 (s, 1H), 8.22 (s, 1H), 8.03 (s, 1H), 7.82 (s, 1H), 7.39 (s, 1H), 3.93 (s, 3H), 3.62 (br t, J=6.28 Hz, 2H), 3.44 (br d, J=6.17 Hz, 4H), 3.02 (br s, 2H), 2.66 (br s, 2H), 2.28 (s, 3H), 1.91 (br d, J=14.99 Hz, 2H), 1.39-1.51 (m, 2H).

### Example 194: (R)—N-(5-((2-(3-hydroxypyrrolidin-1-yl)ethyl)carbamoyl)-3-methylthiophen-2-yl)-2-(1-methyl-1H-pyrazol-4-yl)pyrazolo[5,1-b]thiazole-7-carboxamide

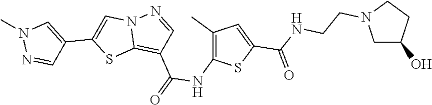 [SMILES: Cc1cc(C(=O)NCCN2CC[C@@H](O)C2)sc1NC(=O)c1cnn2cc(-c3cnn(C)c3)sc12]

To a solution of 4-methyl-5-(2-(1-methyl-1H-pyrazol-4-yl)pyrazolo[5,1-b]thiazole-7-carboxamido)thiophene-2-carboxylic acid (100 mg, 0.26 mmol), (R)-1-(2-aminoethyl)pyrrolidin-3-ol (40 mg, 0.31 mmol) and N-ethyl-N-isopropylpropan-2-amine (0.14 mL, 1.0 mmol) in DMF (4 mL) was added T₃P (411 mg, 0.65 mmol). The resulting mixture was stirred at 25° C. for 16 hours and then concentrated under vacuum to give the crude product, which was purified by preparative high-performance liquid chromatography over column: Boston Green ODS 150*30 mm*5 um to give the title compound (R)—N-(5-((2-(3-hydroxypyrrolidin-1-yl)ethyl)carbamoyl)-3-methylthiophen-2-yl)-2-(1-methyl-1H-pyrazol-4-yl)pyrazolo[5,1-b]thiazole-7-carboxamide (11 mg, 8%) as a yellow solid. LCMS (ESI): mass calcd. for C₂₂H₂₅N₇O₃S₂, 499.6; m/z found, 500.2 [M+H]⁺. ¹H NMR (400 MHz, METHANOL-d₄) δ 8.47 (br s, 1H), 8.25 (s, 1H), 8.06 (s, 1H), 7.86 (s, 1H), 7.43 (s, 1H), 4.43 (br s, 1H), 3.97 (s, 3H), 3.56 (t, J=6.53 Hz, 2H), 2.95-3.06 (m, 2H), 2.90 (br s, 2H), 2.81 (br s, 2H), 2.31 (s, 3H), 2.12-2.23 (m, 1H), 1.83 (br s, 1H).

### Example 195: (S)—N-(5-((2-(3-hydroxypyrrolidin-1-yl)ethyl)carbamoyl)-3-methylthiophen-2-yl)-2-(1-methyl-1H-pyrazol-4-yl)pyrazolo[5,1-b]thiazole-7-carboxamide

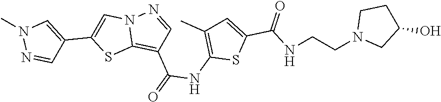 [SMILES: Cc1cc(C(=O)NCCN2CC[C@H](O)C2)sc1NC(=O)c1cnn2cc(-c3cnn(C)c3)sc12]

To a solution of 4-methyl-5-(2-(1-methyl-1H-pyrazol-4-yl)pyrazolo[5,1-b]thiazole-7-carboxamido)thiophene-2-carboxylic acid (110 mg, 0.28 mmol), T₃P (361 mg, 0.57 mmol) and TEA (115 mg, 1.1 mmol) in DMF (6 mL) was added (S)-1-(2-aminoethyl)pyrrolidin-3-ol (37 mg, 0.28 mmol). The resulting mixture was stirred at room temperature for 16 h and then concentrated under vacuum to give the crude product, which was purified by preparative high-performance liquid chromatography over column: Phenomenex Synergi C18 150*30 mm*4 um to give the title compound (S)—N-(5-((2-(3-hydroxypyrrolidin-1-yl)ethyl)carbamoyl)-3-methylthiophen-2-yl)-2-(1-methyl-1H-pyrazol-4-yl)pyrazolo[5,1-b]thiazole-7-carboxamide (17.4 mg, 12%) as a white solid. LCMS (ESI): mass calcd. for C₂₂H₂₅N₇O₃S₂, 499.6; m/z found, 500.0 [M+H]⁺. ¹H NMR (400 MHz, METHANOL-d₄) δ 8.23-8.29 (m, 1H), 8.02 (s, 1H), 7.82-7.88 (m, 1H), 7.62-7.67 (m, 1H), 7.21 (br s, 1H), 3.76 (s, 3H), 3.12 (br s, 3H), 2.61-2.72 (m, 2H), 2.50-2.58 (m, 2H), 2.43 (br d, J=7.28 Hz, 2H), 2.09 (s, 2H), 1.97 (br dd, J=13.56, 6.50 Hz, 2H), 1.56 (s, 2H).

### Example 196: N-(5-((2-(1-azaspiro[4.4]nonan-1-yl)ethyl)carbamoyl)-3-methylthiophen-2-yl)-2-(1-methyl-1H-pyrazol-4-yl)pyrazolo[5,1-b]thiazole-7-carboxamide

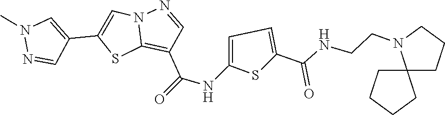 [SMILES: Cn1cc(-c2cn3ncc(C(=O)Nc4ccc(C(=O)NCCN5CCCC56CCCC6)s4)c3s2)cn1]

To a solution of 4-methyl-5-(2-(1-methyl-1H-pyrazol-4-yl)pyrazolo[5,1-b]thiazole-7-carboxamido)thiophene-2-carboxylic acid (55 mg, 0.14 mmol), HATU (97 mg, 0.26 mmol) and N,N-diisopropylethylamine (0.10 mL, 0.57 mmol) in DMF (5 mL) was added 2-(1-azaspiro[4.4]nonan-1-yl)ethanamine (51 mg, 0.21 mmol). The resulting mixture was stirred at room temperature for 12 hours and then concentrated under vacuum to give the crude product, which was purified by preparative high-performance liquid chromatography over column: Welch Xtimate C18 150*30 mm*5 um to give the title compound N-(5-((2-(1-azaspiro[4.4]nonan-1-yl)ethyl)carbamoyl)-3-methylthiophen-2-yl)-2-(1-methyl-1H-pyrazol-4-yl)pyrazolo[5,1-b]thiazole-7-carboxamide (Formic acid salt, 25 mg, 28%) as an off-white solid. LCMS (ESI): mass calcd. for C₂₆H₃₁N₇O₂S₂, 537.2; m/z found, 538.1 [M+H]⁺. ¹H NMR (400 MHz, METHANOL-d₄) δ 8.51-8.44 (m, 2H), 8.26 (s, 1H), 8.07 (s, 1H), 7.86 (s, 1H), 7.45 (s, 1H), 3.97 (s, 3H), 3.72 (br s, 2H), 3.53 (br d, J=19.6 Hz, 4H), 2.33 (s, 3H), 2.18-1.99 (m, 6H), 1.80 (br d, J=10.5 Hz, 6H).

### Example 197: N-(5-((2-(7-oxa-4-azaspiro[2.5]octan-4-yl)ethyl)carbamoyl)-3-methylthiophen-2-yl)-2-(1-methyl-1H-pyrazol-4-yl)pyrazolo[5,1-b]thiazole-7-carboxamide

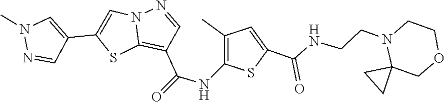 [SMILES: Cc1cc(C(=O)NCCN2CCOCC23CC3)sc1NC(=O)c1cnn2cc(-c3cnn(C)c3)sc12]

To a solution of 4-methyl-5-(2-(1-methyl-1H-pyrazol-4-yl)pyrazolo[5,1-b]thiazole-7-carboxamido)thiophene-2-carboxylic acid (55 mg, 0.14 mmol), HATU (97 mg, 0.26 mmol) and N,N-diisopropylethylamine (0.10 mL, 0.57 mmol) in DMF (5 mL) was added 2-(7-oxa-4-azaspiro[2.5]octan-4-yl)ethanamine (49 mg, 0.21 mmol). The resulting mixture was stirred at room temperature for 12 hours and then concentrated under vacuum to give the crude product, which was purified by preparative high-performance liquid chromatography over column: Welch Xtimate C18 150*30 mm*5 um to give the title compound N-(5-((2-(7-oxa-4-azaspiro[2.5]octan-4-yl)ethyl)carbamoyl)-3-methylthiophen-2-yl)-2-(1-methyl-1H-pyrazol-4-yl)pyrazolo[5,1-b]thiazole-7-carboxamide (28 mg, 35%) as an off-white solid. LCMS (ESI): mass calcd. for C₂₄H₂₇N₇O₃S₂, 525.2; m/z found, 526.1 [M+H]⁺. ¹H NMR (400 MHz, METHANOL-d₄) δ 8.48 (s, 1H), 8.25 (s, 1H), 8.06 (s, 1H), 7.86 (s, 1H), 7.39 (s, 1H), 3.97 (s, 3H), 3.86-3.81 (m, 2H), 3.56 (br s, 2H), 3.45 (br t, J=6.5 Hz, 2H), 3.21-3.11 (m, 4H), 2.31 (s, 3H), 0.85 (s, 2H), 0.72-0.66 (m, 2H).

### Example 198: N-(5-((2-((2S,6R)-2,6-dimethylpiperidin-1-yl)ethyl)carbamoyl)-3-methylthiophen-2-yl)-2-(1-methyl-1H-pyrazol-4-yl)pyrazolo[5,1-b]thiazole-7-carboxamide

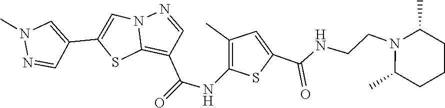 [SMILES: Cc1cc(C(=O)NCCN2[C@H](C)CCC[C@@H]2C)sc1NC(=O)c1cnn2cc(-c3cnn(C)c3)sc12]

To a solution of 4-methyl-5-(2-(1-methyl-1H-pyrazol-3-yl)pyrazolo[5,1-b]thiazole-7-carboxamido)thiophene-2-carboxylic acid (70 mg, 0.18 mmol), 2-((2S,6R)-2,6-dimethylpiperidin-1-yl)ethanamine (31.06 mg, 0.20 mmol) and N,N-diisopropylethylamine (101 mg, 0.78 mmol) in N,N-dimethylformamide (3 mL) was added 2-(3H-[1,2,3]triazolo[4,5-b]pyridin-3-yl)-1,1,3,3-tetramethylisouronium hexafluorophosphate(V) (83.52 mg, 0.22 mmol). The mixture stirred at 50° C. for 2 h and then concentrated under vacuum to give the crude product, which was purified by preparative high-performance liquid chromatography over column: Boston Green ODS 150*30 mm*5 um to give the title compound N-(5-((2-((2S,6R)-2,6-dimethylpiperidin-1-yl)ethyl)carbamoyl)-3-methylthiophen-2-yl)-2-(1-methyl-1H-pyrazol-4-yl)pyrazolo[5,1-b]thiazole-7-carboxamide (12.3 mg, 18%) as a white solid. LCMS (ESI): mass calcd. for C₂₅H₃₁N₇O₂S₂, 525.689; m/z found, 526.1 [M+H]⁺. ¹H NMR (400 MHz, METHANOL-d₄) δ 8.55 (s, 0.19H), 8.46 (s, 1H), 8.23 (s, 1H), 8.05 (s, 1H), 7.84 (s, 1H), 7.38 (s, 1H), 3.95 (s, 3H), 3.52 (br s, 2H), 3.15-3.01 (m, 2H), 2.29 (s, 3H), 1.72 (br s, 2H), 1.54-1.23 (m, 10H).

### Example 199: N-(5-((2-(2-azabicyclo[2.2.2]octan-2-yl)ethyl)carbamoyl)-3-methylthiophen-2-yl)-2-(1-methyl-1H-pyrazol-4-yl)pyrazolo[5,1-b]thiazole-7-carboxamide

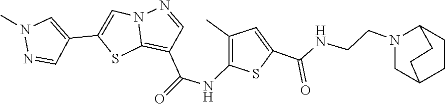 [SMILES: Cc1cc(C(=O)NCCN2CC3CCC2CC3)sc1NC(=O)c1cnn2cc(-c3cnn(C)c3)sc12]

To a solution of 4-methyl-5-(2-(1-methyl-1H-pyrazol-3-yl)pyrazolo[5,1-b]thiazole-7-carboxamido)thiophene-2-carboxylic acid (50 mg, 0.13 mmol), 2-(2-azabicyclo[2.2.2]octan-2-yl)ethanamine (19.9 mg, 0.20 mmol) and N,N-diisopropylethylamine (50.0 mg, 0.39 mmol) in N,N-dimethylformamide (2 mL) was added 2-(3H-[1,2,3]triazolo[4,5-b]pyridin-3-yl)-1,1,3,3-tetramethylisouronium hexafluorophosphate(V) (58.9 mg, 0.16 mmol). The mixture stirred at 50° C. for 2 h and then concentrated under vacuum to give the crude product, which was purified by preparative high-performance liquid chromatography over column: Boston Green ODS 150*30 mm*5 um to give the title compound N-(5-((2-(2-azabicyclo[2.2.2]octan-2-yl)ethyl)carbamoyl)-3-methylthiophen-2-yl)-2-(1-methyl-1H-pyrazol-4-yl)pyrazolo[5,1-b]thiazole-7-carboxamide (25 mg, 35%) as a white solid. LCMS (ESI): mass calcd. for C₂₅H₂₉N₇O₂S₂, 523.673; m/z found, 524.2 [M+H]⁺. ¹H NMR (400 MHz, METHANOL-d₄) δ 8.55 (s, 0.19H), 8.46 (s, 1H), 8.23 (s, 1H), 8.05 (s, 1H), 7.84 (s, 1H), 7.38 (s, 1H), 3.95 (s, 3H), 3.52 (br s, 2H), 3.15-3.01 (m, 2H), 2.29 (s, 3H), 1.72 (br s, 2H), 1.54-1.23 (m, 10H).

### Example 200: N-(5-((2-(3-hydroxypiperidin-1-yl)ethyl)carbamoyl)-3-methylthiophen-2-yl)-2-(1-methyl-1H-pyrazol-4-yl)pyrazolo[5,1-b]thiazole-7-carboxamide

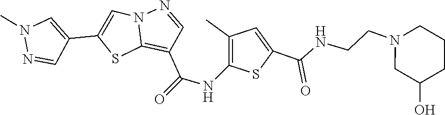 [SMILES: Cc1cc(C(=O)NCCN2CCCC(O)C2)sc1NC(=O)c1cnn2cc(-c3cnn(C)c3)sc12]

To a solution of 4-methyl-5-(2-(1-methyl-1H-pyrazol-3-yl)pyrazolo[5,1-b]thiazole-7-carboxamido)thiophene-2-carboxylic acid (150 mg, 0.39 mmol), 1-(2-aminoethyl)piperidin-3-ol (99.5 mg, 0.43 mmol) and N,N-diisopropylethylamine (150 mg, 1.16 mmol) in N,N-dimethylformamide (5 mL) was added 2-(3H-[1,2,3]triazolo[4,5-b]pyridin-3-yl)-1,1,3,3-tetramethylisouronium hexafluorophosphate(V) (177 mg, 0.47 mmol). The mixture stirred at room temperature for 2 h and then concentrated under vacuum to give the crude product, which was purified by preparative high-performance liquid chromatography over column: Boston Green ODS 150*30 mm*5 um to give the title compound N-(5-((2-(3-hydroxypiperidin-1-yl)ethyl)carbamoyl)-3-methylthiophen-2-yl)-2-(1-methyl-1H-pyrazol-4-yl)pyrazolo[5,1-b]thiazole-7-carboxamide (25 mg, 35%) as a white solid. LCMS (ESI): mass calcd. for C₂₃H₂₇N₇O₃S₂, 513.636; m/z found, 514.1[M+H]⁺. ¹H NMR (400 MHz, METHANOL-d₄) δ 8.45 (br s, 2H), 8.20 (s, 1H), 8.02 (s, 1H), 7.82 (s, 1H), 7.40 (s, 1H), 4.01 (br s, 1H), 3.94 (s, 3H), 3.67 (br t, J=5.9 Hz, 2H), 3.26-3.02 (m, 6H), 2.29 (s, 3H), 2.10 (br s, 1H), 1.88-1.63 (m, 3H).

### Example 201: N-(5-((3-(2,2-dimethylpyrrolidin-1-yl)propyl)carbamoyl)-3-methylthiophen-2-yl)-2-(1-methyl-1H-pyrazol-4-yl)pyrazolo[5,1-b]thiazole-7-carboxamide

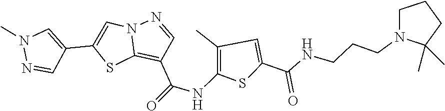

To a solution of 4-methyl-5-(2-(1-methyl-1H-pyrazol-3-yl)pyrazolo[5,1-b]thiazole-7-carboxamido)thiophene-2-carboxylic acid (80.0 mg, 0.21 mmol), 3-(2,2-dimethylpyrrolidin-1-yl)propan-1-amine (50.8 mg, 0.23 mmol) and N,N-diisopropylethylamine (80.1 mg, 0.62 mmol) in N,N-dimethylformamide (4 mL) was added 2-(3H-[1,2,3]triazolo[4,5-b]pyridin-3-yl)-1,1,3,3-tetramethylisouronium hexafluorophosphate(V) (94.2 mg, 0.25 mmol). The mixture stirred at 60° C. for 2 h and then concentrated under vacuum to give the crude product, which was purified by preparative high-performance liquid chromatography over column: Boston Green ODS 150*30 mm*5 um to give the title compound N-(5-((3-(2,2-dimethylpyrrolidin-1-yl)propyl)carbamoyl)-3-methylthiophen-2-yl)-2-(1-methyl-1H-pyrazol-4-yl)pyrazolo[5,1-b]thiazole-7-carboxamide (22.6 mg, 20%) as a yellow solid. LCMS (ESI): mass calcd. for C₂₅H₃₁N₇O₂S₂, 525.689; m/z found, 526.3[M+H]⁺. ¹H NMR (400 MHz, METHANOL-d₄) δ 8.44 (s, 1H), 8.21 (s, 1H), 8.02 (s, 1H), 7.81 (s, 1H), 7.39 (s, 1H), 3.93 (s, 3H), 3.46 (br s, 2H), 3.34-3.30 (m, 2H), 3.11 (br s, 2H), 2.28 (s, 3H), 2.00 (br d, J=6.8 Hz, 6H), 1.37 (br s, 6H).

### Example 202: 2-(1-methyl-1H-pyrazol-4-yl)-N-(3-methyl-5-((2-methyl-2-(pyrrolidin-1-yl)propyl)carbamoyl)thiophen-2-yl)pyrazolo[5,1-b]thiazole-7-carboxamide

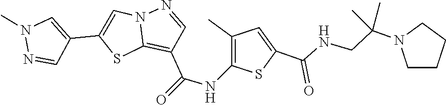 [SMILES: Cc1cc(C(=O)NCC(C)(C)N2CCCC2)sc1NC(=O)c1cnn2cc(-c3cnn(C)c3)sc12]

To a solution of 2-bromo-N-(5-((2-(2,2-dimethylpyrrolidin-1-yl)ethyl)carbamoyl)-3-methylthiophen-2-yl)pyrazolo[5,1-b]thiazole-7-carboxamide (150 mg, 0.29 mmol) and 1-methyl-4-(4,4,5,5-tetramethyl-1,3,2-dioxaborolan-2-yl)-1H-pyrazole (73.4 mg, 0.35 mmol) in 1,4-dioxane (4 mL) and H₂O (1 mL) was added [1,1-bis(diphenylphosphino)ferrocene]palladium(II) chloride dichloromethane complex (23.9 mg, 0.03 mmol) and K₂CO₃ (122 mg, 0.88 mmol) under N₂. The resulting mixture was stirred at 95° C. for 12 h before cooled to 25° C. The reaction mixture was diluted with ethyl acetate (10 mL) and filtered over celite. The filtrate was concentrated to the crude product, which was treated with dichloromethane (10 mL) and water (10 mL). The organic phase was concentrated under reduced pressure to give the crude product, which was purified by preparative high-performance liquid chromatography over column: Boston Prime C18 150*30 mm*5 um to give the title compound 2-(1-methyl-1H-pyrazol-4-yl)-N-(3-methyl-5-((2-methyl-2-(pyrrolidin-1-yl)propyl)carbamoyl)thiophen-2-yl)pyrazolo[5,1-b]thiazole-7-carboxamide (30.6 mg, 18%) as a yellow oil. LCMS (ESI): mass calcd. for C₂₄H₂₉N₇O₂S₂, 511.663; m/z found, 512.3 [M+H]⁺. ¹H NMR (400 MHz, METHANOL-d₄) δ 8.53 (br s, 0.77H), 8.47 (s, 1H), 8.24 (s, 1H), 8.05 (s, 1H), 7.84 (s, 1H), 7.50 (s, 1H), 3.95 (s, 3H), 3.62 (s, 2H), 3.44 (br s, 4H), 2.32 (s, 3H), 2.05 (br s, 4H), 1.40 (s, 6H).

### Example 203: N-(5-((2-(7-azaspiro[3.5]nonan-7-yl)ethyl)carbamoyl)-3-methylthiophen-2-yl)-2-(1-methyl-1H-pyrazol-4-yl)pyrazolo[5,1-b]thiazole-7-carboxamide

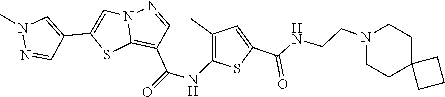 [SMILES: Cc1cc(C(=O)NCCN2CCC3(CCC3)CC2)sc1NC(=O)c1cnn2cc(-c3cnn(C)c3)sc12]

To a solution of 4-methyl-5-(2-(1-methyl-1H-pyrazol-4-yl)pyrazolo[5,1-b]thiazole-7-carboxamido)thiophene-2-carboxylic acid (70 mg, 0.18 mmol), 2-(7-azaspiro[3.5]nonan-7-yl)ethanamine (33.4 mg, 0.20 mmol) and N,N-diisopropylethylamine (70.1 mg, 0.54 mmol) in N,N-dimethylformamide (4 mL) was added 2-(3H-[1,2,3]triazolo[4,5-b]pyridin-3-yl)-1,1,3,3-tetramethylisouronium hexafluorophosphate(V) (103 mg, 0.27 mmol). The mixture stirred at 40° C. for 2 h and then concentrated under vacuum to give the crude product, which was purified by preparative high-performance liquid chromatography over column: Boston Green ODS 150*30 mm*5 um to give the title compound N-(5-((2-(7-azaspiro[3.5]nonan-7-yl)ethyl)carbamoyl)-3-methylthiophen-2-yl)-2-(1-methyl-1H-pyrazol-4-yl)pyrazolo[5,1-b]thiazole-7-carboxamide (31.6 mg, 32%) as a white solid. LCMS (ESI): mass calcd. for C₂₆H₃₁N₇O₂S₂, 537.7; m/z found, 538.3[M+H]⁺. ¹H NMR (400 MHz, METHANOL-d₄) δ 8.47 (s, 1H), 8.24 (s, 1H), 8.05 (s, 1H), 7.84 (s, 1H), 7.42 (s, 1H), 3.95 (s, 3H), 3.69 (t, J=5.8 Hz, 2H), 3.23 (br t, J=5.9 Hz, 6H), 2.30 (s, 3H), 1.97-1.83 (m, 10H).

### Example 204: N-(5-((2-(3,4-dihydroisoquinolin-2 (1H)-yl)ethyl)carbamoyl)-3-methylthiophen-2-yl)-2-(1-methyl-1H-pyrazol-4-yl)pyrazolo[5,1-b]thiazole-7-carboxamide

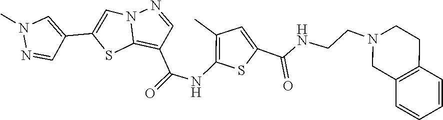 [SMILES: Cc1cc(C(=O)NCCN2CCc3ccccc3C2)sc1NC(=O)c1cnn2cc(-c3cnn(C)c3)sc12]

To a solution of 4-methyl-5-(2-(1-methyl-1H-pyrazol-4-yl)pyrazolo[5,1-b]thiazole-7-carboxamido)thiophene-2-carboxylic acid (50 mg, 0.13 mmol), 2-(3,4-dihydroisoquinolin-2 (1H)-yl)ethanamine (34.1 mg, 0.19 mmol) and N,N-diisopropylethylamine (66.7 mg, 0.52 mmol) in N,N-dimethylformamide (4 mL) was added 2-(3H-[1,2,3]triazolo[4,5-b]pyridin-3-yl)-1,1,3,3-tetramethylisouronium hexafluorophosphate(V) (73.6 mg, 0.19 mmol). The mixture stirred at room temperature for 2 h and then concentrated under vacuum to give the crude product, which was purified by preparative high-performance liquid chromatography over column: Boston Green ODS 150*30 mm*5 um to give the title compound N-(5-((2-(7-azaspiro[3.5]nonan-7-yl)ethyl)carbamoyl)-3-methylthiophen-2-yl)-2-(1-methyl-1H-pyrazol-4-yl)pyrazolo[5,1-b]thiazole-7-carboxamide (26.1 mg, 35%) as a white solid. LCMS (ESI): mass calcd. for C₂₇H₂₇N₇O₂S₂, 545.679; m/z found, 546.3[M+H]⁺. ¹H NMR (400 MHz, METHANOL-d₄) δ 8.45 (s, 0.76H), 8.39 (s, 1H), 8.23 (s, 1H), 8.05 (s, 1H), 7.84 (s, 1H), 7.40 (s, 1H), 7.23-7.10 (m, 4H), 4.06 (s, 2H), 3.95 (s, 3H), 3.70 (t, J=6.1 Hz, 2H), 3.19 (br s, 2H), 3.06 (br s, 4H), 2.28 (s, 3H).

### Example 205: N-(5-((2-(5-azaspiro[3.4]octan-5-yl)ethyl)carbamoyl)-3-ethylthiophen-2-yl)-2-(1-methyl-1H-pyrazol-4-yl)pyrazolo[5,1-b]thiazole-7-carboxamide

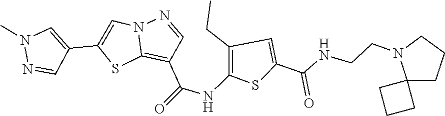 [SMILES: CCc1cc(C(=O)NCCN2CCCC23CCC3)sc1NC(=O)c1cnn2cc(-c3cnn(C)c3)sc12]

### Step a: methyl 4-ethyl-5-nitrothiophene-2-carboxylate

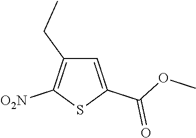

To a solution of methyl 5-nitrothiophene-2-carboxylate (10 g, 53 mmol), propionic acid (7.8 mL, 107 mmol) and ammonium persulfate (30 g, 133 mmol) in mixture of CH₃CN and H₂O (21 mL, 2:1) was added AgNO₃ (18 g, 107 mmol). The reaction mixture was stirred at 80° C. for 2 hours. The reaction mixture was adjusted to pH=8 using Sat.NaHCO₃ and extracted with ethyl acetate (500 mL*3). The combined organic layers were dried with Na₂SO₄, filtered and the filtrates were concentrated under vacuum to afford crude product, which was purified by preparative high-performance liquid chromatography over column: Phenomenex Synergi Max-RP 250*50 mm*10 um to give the title compound methyl 4-ethyl-5-nitrothiophene-2-carboxylate (3.0 g, 26%) as yellow oil. ¹H NMR (400 MHz, CDCl₃) δ 7.59 (s, 1H), 3.91 (s, 3H), 3.05 (q, J=7.5 Hz, 2H), 1.28 (br t, J=7.6 Hz, 3H).

### Step b: methyl 5-amino-4-ethylthiophene-2-carboxylate

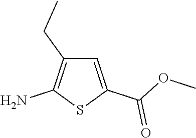

To a solution of methyl 4-ethyl-5-nitrothiophene-2-carboxylate (500 mg, 2.3 mmol) in mixture of MeOH/THF/H₂O (1:1:1, 15 mL) was added Fe (650 mg, 12 mmol) and NH₄Cl (621 mg, 12 mmol). Then the reaction mixture was stirred at 70° C. for 1 hour. After cooling down room temperature, the mixture was filtered off and the filtrate was evaporated give crude product, which was purified by column chromatography over silica gel (eluent: petroleum ether/ethyl acetate=3:1) to give the title compound methyl 5-amino-4-ethylthiophene-2-carboxylate (350 mg, 81%) as a pale brown solid. ¹H NMR (400 MHz, CDCl₃) δ 7.35 (s, 1H), 3.99 (br s, 2H), 3.74 (s, 3H), 2.31 (q, J=7.6 Hz, 2H), 1.13 (t, J=7.6 Hz, 3H).

### Step c: methyl 5-(2-bromopyrazolo[5,1-b]thiazole-7-carboxamido)-4-ethylthiophene-2-carboxylate

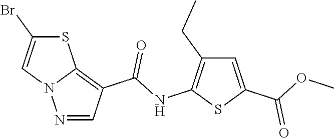 [SMILES: CCc1cc(C(=O)OC)sc1NC(=O)c1cnn2cc(Br)sc12]

To a solution of 2-bromopyrazolo[5,1-b]thiazole-7-carboxylic acid (470 mg, 1.9 mmol) in SOCl₂ (3 mL) was stirred at 70° C. for 2 hours and then concentrated under vacuum to give the crude product 2-bromopyrazolo[5,1-b]thiazole-7-carbonyl chloride (500 mg, 99%) as a pale yellow solid. To a solution of methyl 5-amino-4-ethylthiophene-2-carboxylate (350 mg, 1.9 mmol) and Et₃N (574 mg, 5.7 mmol) in THE (5 mL) was added 2-bromopyrazolo[5,1-b]thiazole-7-carbonyl chloride (502 mg, 1.9 mmol) at room temperature. The reaction mixture was stirred at 80° C. for 16 hours. Then H₂O (10 mL) was added. The mixture was extracted with EtOAc (40 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=3:1) to give the title compound methyl 5-(2-bromopyrazolo[5,1-b]thiazole-7-carboxamido)-4-ethylthiophene-2-carboxylate (300 mg, 38%) as an off white solid. LCMS (ESI): mass calcd. for C₁₄H₁₂BrN₃O₃S₂, 412.9; m/z found, 414.0, 416.0 [M+H]⁺. ¹H NMR (400 MHz, CDCl₃) δ 8.11 (br s, 1H), 7.91 (s, 1H), 7.88 (br s, 1H), 7.61 (s, 1H), 3.88 (s, 3H), 2.64 (q, J=7.7 Hz, 2H), 1.34 (t, J=7.5 Hz, 3H).

### Step d: methyl 4-ethyl-5-(2-(1-methyl-1H-pyrazol-4-yl)pyrazolo[5,1-b]thiazole-7-carboxamido)thiophene-2-carboxylate

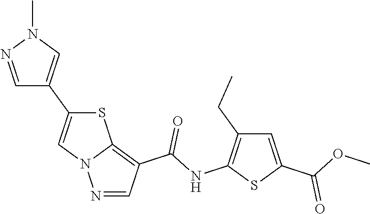 [SMILES: CCc1cc(C(=O)OC)sc1NC(=O)c1cnn2cc(-c3cnn(C)c3)sc12]

To a mixture of 5-(2-bromopyrazolo[5,1-b]thiazole-7-carboxamido)-4-ethylthiophene-2-carboxylate (440 mg, 1.0 mmol), 1-methyl-4-(4,4,5,5-tetramethyl-1,3,2-dioxaborolan-2-yl)-1H-pyrazole (256 mg, 1.2 mmol) and Pd(dppf)Cl₂CH₂Cl₂ (134 mg, 0.16 mmol) in mixture of THF/H₂O (4:1, 40 mL) was added K₂CO₃ (426 mg, 3.1 mmol). The mixture was purged with N₂ with 2 minutes. Then the reaction mixture was stirred at 100° C. for 15 hours. After cooling down room temperature, the mixture was filtered off and the filtrate was evaporated give crude product, which was purified by column chromatography over silica gel (eluent: petroleum ether/ethyl acetate=1:1) to give the title compound methyl 4-ethyl-5-(2-(1-methyl-1H-pyrazol-4-yl)pyrazolo[5,1-b]thiazole-7-carboxamido)thiophene-2-carboxylate (100 mg, 22%) as a pale brown solid. LCMS (ESI): mass calcd. for C₁₈H₁₇N₅O₃S₂, 415.1; m/z found, 416.1 [M+H]⁺.

### Step e: 4-ethyl-5-(2-(1-methyl-1H-pyrazol-4-yl)pyrazolo[5,1-b]thiazole-7-carboxamido)thiophene-2-carboxylic acid

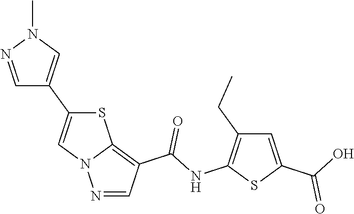 [SMILES: CCc1cc(C(=O)O)sc1NC(=O)c1cnn2cc(-c3cnn(C)c3)sc12]

To a solution of methyl 4-ethyl-5-(2-(1-methyl-1H-pyrazol-4-yl)pyrazolo[5,1-b]thiazole-7-carboxamido)thiophene-2-carboxylate (156 mg, 0.35 mmol) in mixture of THF/MeOH (1:1, 8 mL) was added aqueous NaOH (0.71 mL, 1.4 mmol). Then the reaction mixture was stirred at 70° C. for 15 hours. The reaction mixture was concentrated in vacuo to give crude as a brown solid. Then H₂O (5 mL) was added. The mixture was adjusted to pH-3 with 1N HCl. The mixture was filtered. The filter cake was washed with H₂O (10 mL), dried in vacuo to give 4-ethyl-5-(2-(1-methyl-1H-pyrazol-4-yl)pyrazolo[5,1-b]thiazole-7-carboxamido)thiophene-2-carboxylic acid (146 mg, 92%) as a pale brown solid. LCMS (ESI): mass calcd. for C₁₇H₁₅N5O₃S₂, 401.1; m/z found, 402.1 [M+H]⁺.

### Step f: N-(5-((2-(5-azaspiro[3.4]octan-5-yl)ethyl)carbamoyl)-3-ethylthiophen-2-yl)-2-(1-methyl-1H-pyrazol-4-yl)pyrazolo[5,1-b]thiazole-7-carboxamide

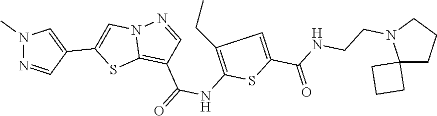 [SMILES: CCc1cc(C(=O)NCCN2CCCC23CCC3)sc1NC(=O)c1cnn2cc(-c3cnn(C)c3)sc12]

To a solution of 4-ethyl-5-(2-(1-methyl-1H-pyrazol-4-yl)pyrazolo[5,1-b]thiazole-7-carboxamido)thiophene-2-carboxylic acid (136 mg, 0.30 mmol), HATU (208 mg, 0.55 mmol) and N,N-diisopropylethylamine (0.26 mL, 1.5 mmol) in DMF (6 mL) was added 2-(5-azaspiro[3.4]octan-5-yl)ethanamine (70 mg, 0.46 mmol). The resulting mixture was stirred at room temperature for 2 hours and then concentrated under vacuum to give the crude product, which was purified by preparative high-performance liquid chromatography over column: Boston Prime C18 150*30 mm*5 um to give the title compound N-(5-((2-(5-azaspiro[3.4]octan-5-yl)ethyl)carbamoyl)-3-ethylthiophen-2-yl)-2-(1-methyl-1H-pyrazol-4-yl)pyrazolo[5,1-b]thiazole-7-carboxamide (50 mg, 29%) as a pale brown solid. LCMS (ESI): mass calcd. for C₂₆H₃₁N₇O₂S₂, 537.2; m/z found, 538.3 [M+H]⁺. ¹H NMR (400 MHz, DMSO-d₆) δ 10.31 (br s, 1H), 8.58 (br s, 2H), 8.27 (br s, 1H), 8.21 (s, 1H), 7.89 (s, 1H), 7.53 (s, 1H), 3.89 (s, 3H), 2.67 (br d, J=7.0 Hz, 6H), 2.57-2.54 (m, 2H), 2.13 (br d, J=6.7 Hz, 2H), 1.85 (br s, 2H), 1.74-1.55 (m, 6H), 1.20 (br t, J=7.3 Hz, 3H).

### Example 206: N-(5-((2-(5-azaspiro[3.4]octan-5-yl)ethyl)carbamoyl)-3-cyanothiophen-2-yl)-2-(1-methyl-1H-pyrazol-4-yl)pyrazolo[5,1-b]thiazole-7-carboxamide

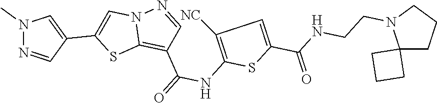 [SMILES: Cn1cc(-c2cn3ncc(C(=O)Nc4sc(C(=O)NCCN5CCCC56CCC6)cc4C#N)c3s2)cn1]

### Step a: methyl 5-(2-bromopyrazolo[5,1-b]thiazole-7-carboxamido)-4-cyanothiophene-2-carboxylate

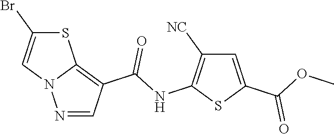 [SMILES: COC(=O)c1cc(C#N)c(NC(=O)c2cnn3cc(Br)sc23)s1]

To a solution of 2-bromopyrazolo[5,1-b]thiazole-7-carboxylic acid (600 mg, 2.4 mmol) in SOCl₂ (10 mL) was stirred at 70° C. for 4 hours and then concentrated under vacuum to give the crude product 2-bromopyrazolo[5,1-b]thiazole-7-carbonyl chloride (650 mg, 100%) as a pale yellow solid. To a solution of methyl 5-amino-4-cyanothiophene-2-carboxylate (400 mg, 2.2 mmol) and Na₂CO₃ (931 mg, 8.8 mmol) in THE (30 mL) was added 2-bromopyrazolo[5,1-b]thiazole-7-carbonyl chloride (641 mg, 2.4 mmol) at room temperature. The reaction mixture was stirred at 70° C. for 16 hours. Then H₂O (80 mL) was added. The mixture was extracted with EtOAc (300 mL*3). The organic extracts were dried over anhydrous Na₂SO₄ and filtered. The filtrate was concentrated under reduced pressure to give crude product, which was triturated with MeOH (20 mL). The mixture was filtered. The filter cake was dried in vacuo to give the title compound methyl 5-(2-bromopyrazolo[5,1-b]thiazole-7-carboxamido)-4-cyanothiophene-2-carboxylate (390 mg, 43%) as a pale brown solid. LCMS (ESI): mass calcd. for C₁₃H₇BrN₄O₃S₂, 409.9; m/z found, 411.0, 413.0 [M+H]⁺.

### Step b: methyl 4-cyano-5-(2-(1-methyl-1H-pyrazol-4-yl)pyrazolo[5,1-b]thiazole-7-carboxamido)thiophene-2-carboxylate

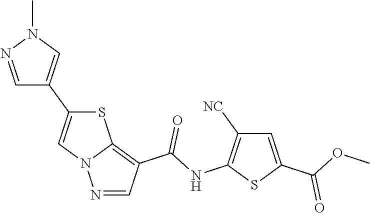 [SMILES: COC(=O)c1cc(C#N)c(NC(=O)c2cnn3cc(-c4cnn(C)c4)sc23)s1]

To a mixture of methyl 5-(2-bromopyrazolo[5,1-b]thiazole-7-carboxamido)-4-cyanothiophene-2-carboxylate (580 mg, 1.4 mmol), 1-methyl-4-(4,4,5,5-tetramethyl-1,3,2-dioxaborolan-2-yl)-1H-pyrazole (352 mg, 1.7 mmol) and Pd(dppf)Cl₂CH₂Cl₂ (184 mg, 0.23 mmol) in mixture of THF/H₂O (4:1, 55 mL) was added K₂CO₃ (585 mg, 4.2 mmol). The mixture was purged with N₂ with 2 minutes. Then the reaction mixture was stirred at 100° C. for 15 hours. After cooling down room temperature, the mixture was filtered off and the filtrate was evaporated to give crude product, which was triturated with MeOH (20 mL). The mixture was filtered. The filter cake was dried in vacuo to give the title compound methyl 4-cyano-5-(2-(1-methyl-1H-pyrazol-4-yl)pyrazolo[5,1-b]thiazole-7-carboxamido)thiophene-2-carboxylate (340 mg, 29%) as a brown solid. LCMS (ESI): mass calcd. for C₁₇H₁₂BN₆O₃S₂, 412.0; m/z found, 413.1 [M+H]⁺

### Step c: N-(5-((2-(5-azaspiro[3.4]octan-5-yl)ethyl)carbamoyl)-3-cyanothiophen-2-yl)-2-(1-methyl-1H-pyrazol-4-yl)pyrazolo[5,1-b]thiazole-7-carboxamide

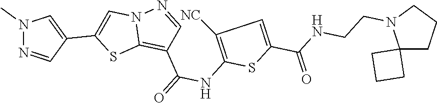 [SMILES: Cn1cc(-c2cn3ncc(C(=O)Nc4sc(C(=O)NCCN5CCCC56CCC6)cc4C#N)c3s2)cn1]

To a mixture of methyl 4-cyano-5-(2-(1-methyl-1H-pyrazol-4-yl)pyrazolo[5,1-b]thiazole-7-carboxamido)thiophene-2-carboxylate (140 mg, 0.34 mmol) and 2-(5-azaspiro[3.4]octan-5-yl)ethanamine (157 mg, 1.0 mmol) in THE (9 mL) was added AlMe₃ (0.68 mL, 1.4 mmol, 2M in toluene) under N₂ at 0° C. The resulting mixture was stirred at 60° C. for 36 hours before cooled down room temperature. The reaction mixture was quenched with MeOH (15 mL) and filtered over celite. The filtrate was concentrated to the crude product. The crude was purified by preparative high-performance liquid chromatography over column: Phenomenex Gemini-NX 80*40 mm*3 um to give the title compound N-(5-((2-(5-azaspiro[3.4]octan-5-yl)ethyl)carbamoyl)-3-cyanothiophen-2-yl)-2-(1-methyl-1H-pyrazol-4-yl)pyrazolo[5,1-b]thiazole-7-carboxamide (36 mg, 21%) as a yellow solid. LCMS (ESI): mass calcd. for C₂₅H₂₆N₈O₂S₂, 534.2; m/z found, 535.2 [M+H]⁺. ¹H NMR (400 MHz, DMSO-d₆) δ 8.40 (s, 1H), 8.08 (s, 1H), 7.97 (s, 1H), 7.84 (br t, J=5.8 Hz, 1H), 7.80 (s, 1H), 7.51 (s, 1H), 3.90 (s, 3H), 3.25 (br d, J=7.3 Hz, 2H), 2.70-2.60 (m, 4H), 2.15-2.09 (m, 2H), 1.87-1.81 (m, 2H), 1.72-1.65 (m, 2H), 1.64-1.56 (m, 4H).

### Example 207: N-(5-((2-(2,2-dimethylpyrrolidin-1-yl)ethyl)carbamoyl)-2-fluorophenyl)-2-(1-methyl-1H-pyrazol-4-yl)pyrazolo[5,1-b]thiazole-7-carboxamide

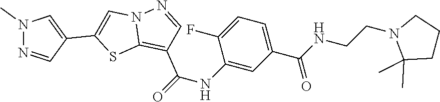 [SMILES: Cn1cc(-c2cn3ncc(C(=O)Nc4cc(C(=O)NCCN5CCCC5(C)C)ccc4F)c3s2)cn1]

### Step a: 3-amino-N-(2-(2,2-dimethylpyrrolidin-1-yl)ethyl)-4-fluorobenzamide

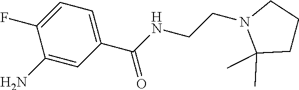 [SMILES: CC1(C)CCCN1CCNC(=O)c1ccc(F)c(N)c1]

To a solution of 3-amino-4-fluorobenzoic acid (500 mg, 3.23 mmol) in DMF (8 mL) was added DIEA (2.1 mL, 12.9 mmol), 2-(2,2-dimethylpyrrolidin-1-yl)ethan-1-amine (504 mg, 3.5 mmol) and HATU (2.4 g, 6.4 mmol), The resulting mixture was heated at 90° C. and stirred for 12 hours. The mixture was concentrated under vacuum to afford a black solid, the crude product was purified by high-performance liquid chromatography over Boston Prime C18 150*30 mm*5 um. The pure fractions were collected, and the solvent was evaporated to give the desired product as a white solid. The yellow solid was dissolved in water and acetonitrile, then dried by lyophilized to give 3-amino-N-(2-(2,2-dimethylpyrrolidin-1-yl)ethyl)-4-fluorobenzamide (90 mg, 20%) as a white solid. LCMS (ESI): mass for C₁₅H₂₂FN₃O: 279.4; m/z found: 280.2 [M+H]⁺.

### Step b: 2-bromo-N-(5-((2-(2,2-dimethylpyrrolidin-1-yl)ethyl)carbamoyl)-2-fluorophenyl)pyrazolo[5,1-b]thiazole-7-carboxamide

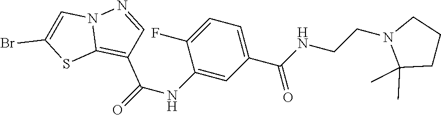 [SMILES: CC1(C)CCCN1CCNC(=O)c1ccc(F)c(NC(=O)c2cnn3cc(Br)sc23)c1]

To a solution of 2-bromopyrazolo[5,1-b]thiazole-7-carboxylic acid (69.87 mg, 0.247 mmol) in SOCl₂ (4 mL) was added to the vial. The reaction mixture was stirred at 90° C. for 2 hours. Then solvent was evaporated to give the residue. To the mixture of 3-amino-N-(2-(2,2-dimethylpyrrolidin-1-yl)ethyl)-4-fluorobenzamide (76 mg, 0.272 mmol) in THE (4 mL) was added the residue and DIEA (131.7 μL, 0.742 mmol). The reaction mixture was stirred at 25° C. for 12 hours. Then solvent was evaporated to give 2-bromo-N-(5-((2-(2,2-dimethylpyrrolidin-1-yl)ethyl)carbamoyl)-2-fluorophenyl)pyrazolo[5,1-b]thiazole-7-carboxamide (136 mg, 57%) as yellow oil. LCMS (ESI): mass for C₂₁H₂₃BrFN₅O₂F: 508.4; m/z found: 510.0 [M+H]⁺.

### Step c: N-(5-((2-(2,2-dimethylpyrrolidin-1-yl)ethyl)carbamoyl)-2-fluorophenyl)-2-(1-methyl-1H-pyrazol-4-yl)pyrazolo[5,1-b]thiazole-7-carboxamide

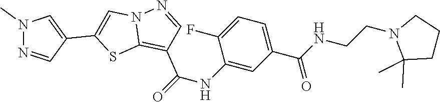 [SMILES: Cn1cc(-c2cn3ncc(C(=O)Nc4cc(C(=O)NCCN5CCCC5(C)C)ccc4F)c3s2)cn1]

To a solution of 2-bromo-N-(5-((2-(2,2-dimethylpyrrolidin-1-yl)ethyl)carbamoyl)-2-fluorophenyl)pyrazolo[5,1-b]thiazole-7-carboxamide (120 mg, 0.136 mmol) in the mixture of dioxane (8 mL) was added 1-methyl-4-(4,4,5,5-tetramethyl-1,3,2-dioxaborolan-2-yl)-1H-pyrazole (56.62 mg, 0.272 mmol), aq.K₂CO₃ (272.2 μL, 2 M) and [1,1′-bis(diphenylphosphino)freeocene]dichloropalladium(II) (11.1 mg, 0.014 mmol) under N₂. The resulting mixture was heated at 90° C. and stirred for 12 hours. LCMS showed most of the starting material was consumed and the desired mass was detected. The mixture was concentrated under vacuum to afford a black solid, which was purified by preparative high-performance liquid chromatography over column: Xtimate C18 100*30 mm*3 um to give the final compound. The fractions were collected and the solvent was removed to give N-(5-((2-(2,2-dimethylpyrrolidin-1-yl)ethyl)carbamoyl)-2-fluorophenyl)-2-(1-methyl-11H-pyrazol-4-yl)pyrazolo[5,1-b]thiazole-7-carboxamide (9 mg, 10%) as a white solid. LCMS (ESI): mass for C₂₅H₂₈FN₇O₂S: 509.6; m/z found: 510.3 [M+H]⁺. ¹H NMR (400 MHz, METHANOL-d₄) δ 8.41 (s, 1H), 8.40-8.33 (m, 2H), 8.21 (s, 1H), 8.01 (s, 1H), 7.80 (s, 1H), 7.74 (ddd, J=2.3, 4.6, 8.5 Hz, 1H), 7.32 (dd, J=8.7, 10.0 Hz, 1H), 3.92 (s, 3H), 3.74 (br t, J=6.1 Hz, 2H), 3.61 (br s, 2H), 3.33 (br s, 2H), 2.18-2.06 (m, 2H), 2.06-1.98 (m, 2H), 1.38 (s, 6H).

### Example 208: N-(5-((2-((1R,4S)-2-azabicyclo[2.2.1]heptan-2-yl)ethyl)carbamoyl)-2-methylpyridin-3-yl)-2-(1,5-dimethyl-1H-pyrazol-4-yl)pyrazolo[5,1-b]thiazole-7-carboxamide

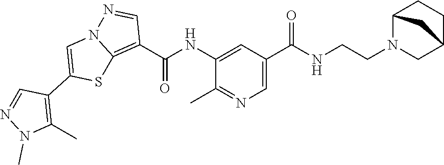 [SMILES: Cc1ncc(C(=O)NCCN2C[C@H]3CC[C@@H]2C3)cc1NC(=O)c1cnn2cc(-c3cnn(C)c3C)sc12]

To a solution of 5-(2-(1,5-dimethyl-1H-pyrazol-4-yl)pyrazolo[5,1-b]thiazole-7-carboxamido)-6-methylnicotinic acid (90 mg, 0.17 mmol) in DMF (7 mL) was added 2-((1R,4S)-2-azabicyclo[2.2.1]heptan-2-yl)ethanamine (70 mg, 0.51 mmol), DIPEA (0.12 mL, 0.69 mmol) and HATU (100 mg, 0.26 mmol) at room temperature. The mixture was stirred at room temperature for 15 minutes. The reaction mixture was stirred at room temperature for 5 hours. The reaction mixture was filtered and the filtrate was concentrated under vacuum to give crude product, which was purified by preparative high-performance liquid chromatography over column: Phenomenex C18 75*30 mm*3 um and was separated by SFC over column: DAICEL CHIRALPAK AS (250 mm*30 mm, 10 um) to give the title compound 2-(4,4-difluoropiperidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (30 mg, 50%) as an off-white solid. LCMS (ESI): mass calcd. for C₂₆H₃₀N₈O₂S, 518.6; m/z found, 519.3 [M+H]⁺. ¹H NMR (400 MHz, METHANOL-d₄) δ 8.70 (s, 1H), 8.34 (s, 1H), 8.26 (d, J=1.8 Hz, 1H), 8.03 (s, 1H), 7.57 (s, 1H), 3.96 (br s, 1H), 3.76 (s, 3H), 3.68-3.51 (m, 2H), 3.13-2.96 (m, 3H), 2.57 (br s, 1H), 2.53 (s, 3H), 2.38 (s, 3H), 1.90-1.79 (m, 2H), 1.77-1.66 (m, 2H), 1.61 (br d, J=11.3 Hz, 1H), 1.46-1.35 (m, 1H), 1.24-1.17 (m, 1H).

### Example 209: N-(5-((2-(5-azaspiro[3.4]octan-5-yl)ethyl)carbamoyl)-2-methyl-pyridin-3-yl)-2-(1,5-dimethyl-1H-pyrazol-4-yl)pyrazolo[5,1-b]thiazole-7-carboxamide

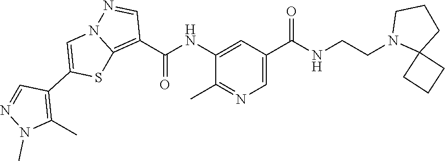 [SMILES: Cc1ncc(C(=O)NCCN2CCCC23CCC3)cc1NC(=O)c1cnn2cc(-c3cnn(C)c3C)sc12]

To a solution of 5-(2-(1,5-dimethyl-1H-pyrazol-4-yl)pyrazolo[5,1-b]thiazole-7-carboxamido)-6-methylnicotinic acid (240 mg, 0.37 mmol) in DMF (8 mL) was added 2-(5-azaspiro[3.4]octan-5-yl)ethanamine (70 mg, 0.47 mmol), DIEA (280 μL, 1.7 mmol) and HATU (180 mg, 0.48 mmol). The resulting mixture was heated at room temperature and stirred for 2 hours. The reaction mixture was filtered and the filtrate was concentrated under vacuum to give crude product, which was purified by preparative by preparative high-performance liquid chromatography over column: Phenomenex C18 75*30 mm*3 um to give the title compound N-(5-((2-(5-azaspiro[3.4]octan-5-yl)ethyl)carbamoyl)-2-methylpyridin-3-yl)-2-(1,5-dimethyl-1H-pyrazol-4-yl)pyrazolo[5,1-b]thiazole-7-carboxamide (30 mg, 15%) as yellow solid. LCMS (ESI): mass calcd. for C₂₇H₃₂N₈O₂S, 532.7; m/z found, 533.3 [M+H]⁺. ¹H NMR (400 MHz, METHANOL-d₄) δ 8.81 (d, J=2.03 Hz, 1H), 8.53 (br s, 1H), 8.36 (d, J=2.03 Hz, 1H), 8.15 (s, 1H), 7.69 (s, 1H), 3.88 (s, 3H), 3.68 (t, J=6.68 Hz, 2H), 3.03-3.19 (m, 4H), 2.64 (s, 3H), 2.50 (s, 3H), 2.35-2.47 (m, 2H), 2.10-2.18 (m, 2H), 1.94-2.02 (m, 2H), 1.76-1.93 (m, 4H).

### Example 210: N-(5-((2-(2-azabicyclo[2.2.1]heptan-2-yl)ethyl)carbamoyl)-2-methylpyridin-3-yl)-2-(1,5-dimethyl-1H-pyrazol-4-yl)pyrazolo[5,1-b]thiazole-7-carboxamide

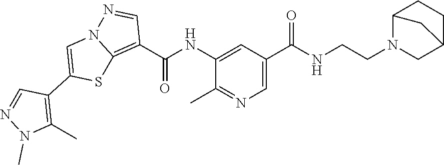 [SMILES: Cc1ncc(C(=O)NCCN2CC3CCC2C3)cc1NC(=O)c1cnn2cc(-c3cnn(C)c3C)sc12]

To a solution of 5-(2-(1,5-dimethyl-1H-pyrazol-4-yl)pyrazolo[5,1-b]thiazole-7-carboxamido)-6-methylnicotinic acid (500 mg, 0.76 mmol), HATU (350 mg, 0.92 mmol) and N,N-diisopropylethylamine (0.51 mL, 3.1 mmol) in N,N-dimethylformamide (6 mL) was added 2-(2-azabicyclo[2.2.1]heptan-2-yl)ethanamine (120 mg, 0.84 mmol). The mixture stirred at room temperature for 1 hour and then concentrated under vacuum to give the crude product, which was purified by preparative high-performance liquid chromatography over column: Xtimate C18 150*40 mm*5 um to give the title compound N-(5-((2-(2-azabicyclo[2.2.1]heptan-2-yl)ethyl)carbamoyl)-2-methylpyridin-3-yl)-2-(1,5-dimethyl-1H-pyrazol-4-yl)pyrazolo[5,1-b]thiazole-7-carboxamide (FA salt, 42.6 mg, 10%) as a white solid. LCMS (ESI): mass calcd. for C₂₆H₃₀N₈O₂S, 518.6; m/z found, 519.2 [M+H]⁺. ¹H NMR (400 MHz, CD₃OD) δ 8.85-8.79 (m, 1H), 8.60-8.52 (m, 1H), 8.46 (s, 1H), 8.38 (d, J=2.0 Hz, 1H), 8.18-8.13 (m, 1H), 7.72-7.67 (m, 1H), 4.12-4.02 (m, 1H), 3.94-3.84 (m, 3H), 3.81-3.61 (m, 2H), 3.26-3.04 (m, 3H), 2.68 (br s, 1H), 2.65 (s, 3H), 2.57-2.45 (m, 3H), 2.02-1.90 (m, 2H), 1.89-1.77 (m, 2H), 1.76-1.68 (m, 1H), 1.60-1.49 (m, 1H).

### Example 211: N-(5-((2-((1R,4S)-2-azabicyclo[2.2.1]heptan-2-yl)ethyl)carbamoyl)-2-methylpyridin-3-yl)-2-(1,3-dimethyl-1H-pyrazol-4-yl)pyrazolo[5,1-b]thiazole-7-carboxamide

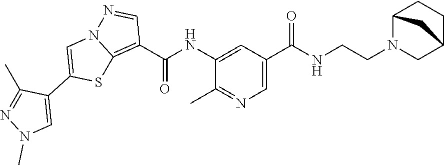 [SMILES: Cc1ncc(C(=O)NCCN2C[C@H]3CC[C@@H]2C3)cc1NC(=O)c1cnn2cc(-c3cn(C)nc3C)sc12]

### Step a: methyl 5-(2-(1,3-dimethyl-1H-pyrazol-4-yl)pyrazolo[5,1-b]thiazole-7-carboxamido)-6-methylnicotinate

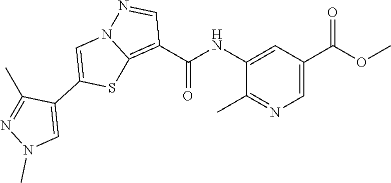 [SMILES: COC(=O)c1cnc(C)c(NC(=O)c2cnn3cc(-c4cn(C)nc4C)sc23)c1]

To a solution of Ethyl 5-(2-bromopyrazolo[5,1-b]thiazole-7-carboxamido)-6-methylnicotinate (1.5 g, 3.6 mmol) and (1,3-dimethyl-1H-pyrazol-4-yl)boronic acid (580 mg, 4.2 mmol) in 1,4-dioxane (120 mL) and H₂O (30 mL) was added [1,1-bis(diphenylphosphino)ferrocene]palladium(II) chloride dichloromethane complex (900 mg, 1.1 mmol) and cesium carbonate (3.6 g, 10.9 mmol) under N₂. The resulting mixture was stirred at 100° C. for 12 h before cooled to 25° C. The reaction mixture was diluted with ethyl acetate (10 mL) and filtered over celite. The filtrate was concentrated to the crude product, which was purified by column chromatography over silica gel (eluent: dichloromethane:Methanol=4:1) to give the title compound to give the title compound 2-(6-aminopyridin-3-yl)-N-(5-((2-(2,2-dimethylpyrrolidin-1-yl)ethyl)carbamoyl)-2-methylpyridin-3-yl)pyrazolo[5,1-b]thiazole-7-carboxamide (650 mg, 43%) as a brown solid. LCMS (ESI): mass calcd. for C₁₉H₁₈N₆O₃S, 410.45; m/z found, 411.2 [M+H]⁺.

### Step b: 5-(2-(1,3-dimethyl-1H-pyrazol-4-yl)pyrazolo[5,1-b]thiazole-7-carboxamido)-6-methylnicotinic acid

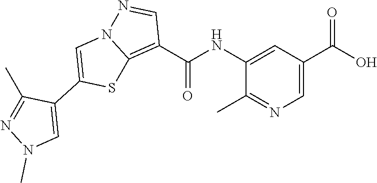 [SMILES: Cc1ncc(C(=O)O)cc1NC(=O)c1cnn2cc(-c3cn(C)nc3C)sc12]

To a solution of methyl 5-(2-(1,3-dimethyl-1H-pyrazol-4-yl)pyrazolo[5,1-b]thiazole-7-carboxamido)-6-methylnicotinate (630 mg, 1.54 mmol) in ethanol (20 mL) was added aq. sodium hydroxide (5 mL, 10.0 mmol) at room-temperature. The reaction mixture was stirred at room temperature for 1 h. The mixture was adjusted to pH=3-4 with HCl (aq, 2 M). The mixture was filtered and washed with water (10 mL×3). The solid was evaporated under vacuum to give desired product 5-(2-(1,3-dimethyl-1H-pyrazol-4-yl)pyrazolo[5,1-b]thiazole-7-carboxamido)-6-methylnicotinic acid (550 mg, 90%) as white solid. LCMS (ESI): mass calcd. for C₁₈H₁₆N₆O₃S, 396.423; m/z found, 397.1 [M+H]⁺

### Step c: N-(5-((2-((1R,4S)-2-azabicyclo[2.2.1]heptan-2-yl)ethyl)carbamoyl)-2-methylpyridin-3-yl)-2-(1,3-dimethyl-1H-pyrazol-4-yl)pyrazolo[5,1-b]thiazole-7-carboxamide

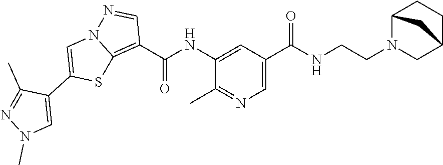 [SMILES: Cc1ncc(C(=O)NCCN2C[C@H]3CC[C@@H]2C3)cc1NC(=O)c1cnn2cc(-c3cn(C)nc3C)sc12]

To a solution of 5-(2-(1,3-dimethyl-1H-pyrazol-4-yl)pyrazolo[5,1-b]thiazole-7-carboxamido)-6-methylnicotinic acid (100 mg, 0.25 mmol), 2-((1R,4S)-2-azabicyclo[2.2.1]heptan-2-yl)ethanamine (40 mg, 0.29 mmol) and N,N-diisopropylethylamine (100 mg, 0.76 mmol) in DMF (5 mL) was added HATU (140 mg, 0.38 mmol). The resulting mixture was stirred at room temperature for 2 hours and then concentrated under vacuum to give the crude product, which was purified by preparative high-performance liquid chromatography over column: Boston Green ODS 150*30 mm*5 um to give the title compound N-(5-((2-((1R,4S)-2-azabicyclo[2.2.1]heptan-2-yl)ethyl)carbamoyl)-2-methylpyridin-3-yl)-2-(1,3-dimethyl-1H-pyrazol-4-yl)pyrazolo[5,1-b]thiazole-7-carboxamide (25 mg, 17%) as a white solid. LCMS (ESI): mass calcd. for C₂₆H₃₀N₈O₂S, 518.634; m/z found, 519.2 [M+H]⁺. ¹H NMR (400 MHz, METHANOL-d₄) δ 8.82 (d, J=2.0 Hz, 1H), 8.56 (s, 1H), 8.38 (d, J=2.0 Hz, 1H), 8.14 (s, 1H), 7.94 (s, 1H), 4.10 (br s, 1H), 3.89 (s, 3H), 3.82-3.63 (m, 2H), 3.42-3.36 (m, 1H), 3.28-3.08 (m, 3H), 2.70 (br s, 1H), 2.65 (s, 3H), 2.43 (s, 3H), 2.00-1.71 (m, 5H), 1.61-1.48 (m, 1H).

### Example 212: N-(5-((2-(2-azabicyclo[2.2.1]heptan-2-yl)ethyl)carbamoyl)-2-methylpyridin-3-yl)-2-(1,3-dimethyl-1H-pyrazol-4-yl)pyrazolo[5,1-b]thiazole-7-carboxamide

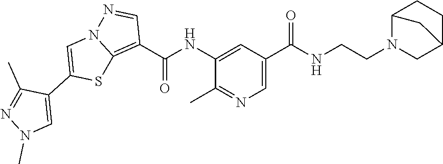 [SMILES: Cc1ncc(C(=O)NCCN2CC3CCC2C3)cc1NC(=O)c1cnn2cc(-c3cn(C)nc3C)sc12]

To a solution of 5-(2-(1,3-dimethyl-1H-pyrazol-4-yl)pyrazolo[5,1-b]thiazole-7-carboxamido)-6-methylnicotinic acid (500 mg, 1.3 mmol), HATU (590 mg, 1.5 mmol) and N,N-diisopropylethylamine (0.9 mL, 5.4 mmol) in N,N-dimethylformamide (25 mL) was added 2-(2-azabicyclo[2.2.1]heptan-2-yl)ethanamine (230 mg, 1.6 mmol). The mixture stirred at room temperature for 2 hours and then concentrated under vacuum to give the crude product, which was purified by preparative high-performance liquid chromatography over column: Boston Green ODS 150*30 mm*5 um to give the title compound N-(5-((2-(2-azabicyclo[2.2.1]heptan-2-yl)ethyl)carbamoyl)-2-methylpyridin-3-yl)-2-(1,3-dimethyl-1H-pyrazol-4-yl)pyrazolo[5,1-b]thiazole-7-carboxamide (FA salt, 45.4 mg, 6%) as a white solid. LCMS (ESI): mass calcd. for C₂₆H₃₀N₈O₂S, 518.6; m/z found, 519.3 [M+H]⁺. ¹H NMR (400 MHz, METHANOL-d₄) δ 8.70 (d, J=2.0 Hz, 1H), 8.33 (s, 1H), 8.24 (d, J=2.0 Hz, 1H), 8.02 (s, 1H), 7.82 (s, 1H), 3.77 (s, 3H), 3.13-2.91 (m, 3H), 2.78 (br s, 1H), 2.52 (s, 4H), 2.31 (s, 3H), 1.79 (br d, J=10.7 Hz, 2H), 1.64 (br s, 2H), 1.52 (br d, J=10.6 Hz, 1H), 1.38 (br d, J=11.3 Hz, 1H), 1.19 (br s, 2H), 0.79 (br d, J=7.5 Hz, 1H).

### Example 213: N-(5-((2-(5-azaspiro[3.4]octan-5-yl)ethyl)carbamoyl)-2-methylpyridin-3-yl)-2-(1,3-dimethyl-1H-pyrazol-4-yl)pyrazolo[5,1-b]thiazole-7-carboxamide

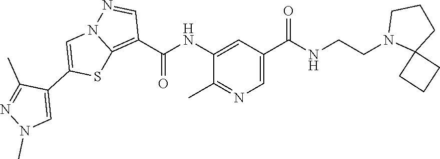 [SMILES: Cc1ncc(C(=O)NCCN2CCCC23CCC3)cc1NC(=O)c1cnn2cc(-c3cn(C)nc3C)sc12]

To a solution of 5-(2-(1,3-dimethyl-1H-pyrazol-4-yl)pyrazolo[5,1-b]thiazole-7-carboxamido)-6-methylnicotinic acid (100 mg, 0.25 mmol), 2-(5-azaspiro[3.4]octan-5-yl)ethanamine (50 mg, 0.28 mmol) and N,N-diisopropylethylamine (100 mg, 0.76 mmol) in DMF (5 mL) was added HATU(140 mg, 0.38 mmol). The resulting mixture was stirred at room temperature for 2 hours and then concentrated under vacuum to give the crude product, which was purified by preparative high-performance liquid chromatography over column: Boston Green ODS 150*30 mm*5 um to give the title compound N-(5-((2-(5-azaspiro[3.4]octan-5-yl)ethyl)carbamoyl)-2-methylpyridin-3-yl)-2-(1,3-dimethyl-1H-pyrazol-4-yl)pyrazolo[5,1-b]thiazole-7-carboxamide (34.6 mg, 23%) as a white solid. LCMS (ESI): mass calcd. for C₂₇H₃₂N₈O₂S, 532.66; m/z found, 533.3 [M+H]⁺. ¹H NMR (400 MHz, METHANOL-d₄) δ 8.81 (d, J=2.0 Hz, 1H), 8.44 (s, 1H), 8.36 (d, J=1.9 Hz, 1H), 8.12 (s, 1H), 7.93 (s, 1H), 3.89 (s, 3H), 3.68 (t, J=6.7 Hz, 2H), 3.18-3.06 (m, 4H), 2.64 (s, 3H), 2.47-2.39 (m, 5H), 2.17-2.10 (m, 2H), 2.02-1.94 (m, 2H), 1.93-1.77 (m, 4H).

### Example 214: N-(5-((2-(5-azaspiro[3.4]octan-5-yl)ethyl)carbamoyl)-2-methylpyridin-3-yl)-2-(1,3,5-trimethyl-1H-pyrazol-4-yl)pyrazolo[5,1-b]thiazole-7-carboxamide

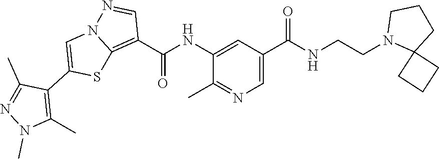 [SMILES: Cc1ncc(C(=O)NCCN2CCCC23CCC3)cc1NC(=O)c1cnn2cc(-c3c(C)nn(C)c3C)sc12]

To a solution of 2-(5-azaspiro[3.4]octan-5-yl) ethanamine (40 mg, 0.259 mmol) in DMF (3 mL) was added 6-methyl-5-(2-(1,3,5-trimethyl-1H-pyrazol-4-yl)pyrazolo[5,1-b]thiazole-7-carboxamido)nicotinic acid (100 mg, 0.181 mmol), DIEA (125 μL, 0.756 mmol) and HATU(90 mg, 0.224 mmol). The resulting mixture was stirred at room temperature for 2 h. The reaction mixture was concentrated under vacuum to give product as brown solid. The mixture was purified by column chromatography over 4 g silica gel (eluent: DCM/MeOH=100/0-80/20), and the fractions were collected and monitored by LCMS. The fractions were collected, and the solvent was removed to give a brown solid, which was purified by preparative high-performance liquid chromatography over column: Phenomenex C18 80*40 mm*3 um to give the final compound. The fractions were collected and the solvent was removed to give N-(5-((2-(5-azaspiro[3.4]octan-5-yl)ethyl)carbamoyl)-2-methylpyridin-3-yl)-2-(1,3,5-trimethyl-1H-pyrazol-4-yl)pyrazolo[5,1-b]thiazole-7-carboxamide (34.7 mg, 32%) as an off-white solid. LCMS (ESI): mass for C₂₈H₃₄N₈O₂S: 546.7; m/z found: 547.3 [M+H]⁺. ¹H NMR (400 MHz, CD₃OD) δ 8.91-8.79 (m, 1H), 8.73-8.55 (m, 1H), 8.42-8.26 (m, 1H), 8.25-8.08 (m, 1H), 7.79-7.62 (m, 1H), 4.36-4.29 (m, 2H), 3.92-3.82 (m, 3H), 3.82-3.74 (m, 2H), 3.44 (br s, 2H), 3.37 (br s, 3H), 2.90-2.77 (m, 2H), 2.63-2.49 (m, 3H), 2.29-2.18 (m, 2H), 2.09-2.02 (m, 2H), 1.91-1.76 (m, 4H), 1.76-1.63 (m, 2H).

### Example 215: N-(5-(4-(1-azaspiro[3.3]heptan-1-yl)butanamido)-2-methylpyridin-3-yl)-2-(1-methyl-1H-pyrazol-4-yl)pyrazolo[5,1-b]thiazole-7-carboxamide

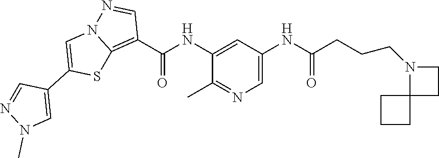 [SMILES: Cc1ncc(NC(=O)CCCN2CCC23CCC3)cc1NC(=O)c1cnn2cc(-c3cnn(C)c3)sc12]

To a solution of N-(5-(4-chlorobutanamido)-2-methylpyridin-3-yl)-2-(1-methyl-1H-pyrazol-4-yl)pyrazolo[5,1-b]thiazole-7-carboxamide (280 mg, 0.61 mmol) and 1-azaspiro[3.3]heptane hemioxalate (390 mg, 1.4 mmol) in CH₃CN (12 mL) was added Et₃N (390 mg, 3.9 mmoL) at room temperature. The reaction mixture was stirred at 60° C. for 15 hours. The reaction mixture was concentrated under vacuum to give the crude product, which was purified by preparative high-performance liquid chromatography over column: Phenomenex C18 80*40 mm*3 um to give the title compound N-(5-(4-(1-azaspiro[3.3]heptan-1-yl)butanamido)-2-methylpyridin-3-yl)-2-(1-methyl-1H-pyrazol-4-yl)pyrazolo[5,1-b]thiazole-7-carboxamide (21 mg, 6.6%) as a white solid. LCMS (ESI): mass calcd. for C₂₆H₃₀N₈O₂S, 518.2; m/z found, 519.3 [M+H]⁺. ¹H NMR (400 MHz, METHANOL-d4) δ 8.57 (d, J=2.3 Hz, 1H), 8.41 (s, 1H), 8.29-8.19 (m, 2H), 8.05 (s, 1H), 7.84 (s, 1H), 3.96 (s, 3H), 3.18 (t, J=7.0 Hz, 2H), 2.60-2.54 (m, 2H), 2.51 (s, 3H), 2.46 (t, J=7.3 Hz, 2H), 2.38-2.20 (m, 4H), 2.04-1.91 (m, 2H), 1.81 (quin, J=7.5 Hz, 2H), 1.74-1.61 (m, 2H).

### Example 216: (S)-2-(1-(2-methoxyethyl)-1H-pyrazol-4-yl)-N-(2-methyl-5-(4-(2-methylpyrrolidin-1-yl)butanamido)pyridin-3-yl)pyrazolo[5,1-b]thiazole-7-carboxamide

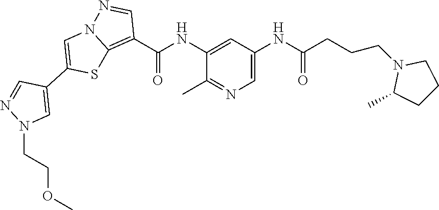 [SMILES: COCCn1cc(-c2cn3ncc(C(=O)Nc4cc(NC(=O)CCCN5CCC[C@@H]5C)cnc4C)c3s2)cn1]

### Step a: N-(5-(4-chlorobutanamido)-2-methylpyridin-3-yl)-2-(1-(2-methoxyethyl)-1H-pyrazol-4-yl)pyrazolo[5,1-b]thiazole-7-carboxamide

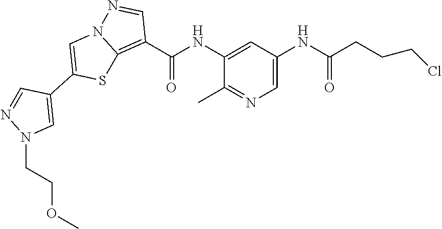 [SMILES: COCCn1cc(-c2cn3ncc(C(=O)Nc4cc(NC(=O)CCCCl)cnc4C)c3s2)cn1]

To a solution of N-(5-amino-2-methylpyridin-3-yl)-2-(1-(2-methoxyethyl)-1H-pyrazol-4-yl)pyrazolo[5,1-b]thiazole-7-carboxamide (200 mg, 0.4 mmol) and NaHCO₃ (2 g, 23.7 mmol) in CHCl₃ (20 mL) and H₂O (20 mL) was added 4-chlorobutanoyl chloride (0.9 mL, 7.9 mmoL) dropwise at 0° C. The reaction was warmed to room temperature and stirred for 2 hours. The reaction mixture was filtered. The filtrate was concentrated in vacuo to give brown solid, which was purified by column chromatography over silica gel (eluent: dichloromethane:methanol=9:1) to give the title compound N-(5-(4-chlorobutanamido)-2-methylpyridin-3-yl)-2-(1-(2-methoxyethyl)-1H-pyrazol-4-yl)pyrazolo[5,1-b]thiazole-7-carboxamide (210 mg, 96%) as a brown oil. LCMS (ESI): mass calcd. for C₂₂H₂₄ClN₇O₃S, 502.0; m/z found, 503.2 [M+H]⁺.

### Step b: (S)-2-(1-(2-methoxyethyl)-1H-pyrazol-4-yl)-N-(2-methyl-5-(4-(2-methylpyrrolidin-1-yl)butanamido)pyridin-3-yl)pyrazolo[5,1-b]thiazole-7-carboxamide

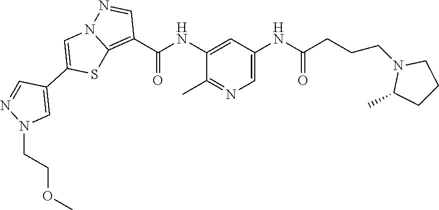 [SMILES: COCCn1cc(-c2cn3ncc(C(=O)Nc4cc(NC(=O)CCCN5CCC[C@@H]5C)cnc4C)c3s2)cn1]

A mixture of N-(5-(4-chlorobutanamido)-2-methylpyridin-3-yl)-2-(1-(2-methoxyethyl)-1H-pyrazol-4-yl)pyrazolo[5,1-b]thiazole-7-carboxamide (200 mg, 0.37 mmol) in (S)-2-methylpyrrolidine (2.7 g, 31.3 mmol) was stirred at 60° C. for 15 hours. The reaction mixture was concentrated in vacuo to give brown solid, which was purified by preparative high-performance liquid chromatography over column: Boston Green ODS 150*30 mm*5 um to give the title compound (S)-2-(1-methyl-1H-pyrazol-4-yl)-N-(2-methyl-5-(4-(2-methylpyrrolidin-1-yl)butanamido)pyridin-3-yl)pyrazolo[5,1-b]thiazole-7-carboxamide (FA salt, 40.2 mg, 18%) as a brown solid. LCMS (ESI): mass calcd. for C₂₇H₃₄N₈O₃S, 550.7; m/z found, 501.3 [M+H]⁺. ¹H NMR (400 MHz, METHANOL-d₄) δ 8.58 (d, J=2.38 Hz, 1H), 8.41 (s, 1H), 8.27 (s, 1H), 8.26 (d, J=2.26 Hz, 1H), 8.09 (s, 1H), 7.87 (s, 1H), 4.37 (t, J=5.13 Hz, 2H), 3.79 (t, J=5.13 Hz, 2H), 3.60-3.73 (m, 1H), 3.39-3.53 (m, 2H), 3.36-3.36 (m, 3H), 2.95-3.19 (m, 2H), 2.62 (t, J=6.85 Hz, 2H), 2.53 (s, 3H), 2.31 (dq, J=13.69, 6.60 Hz, 1H), 2.03-2.17 (m, 4H), 1.68-1.79 (m, 1H), 1.43 (d, J=6.56 Hz, 3H).

### Example 217: N-(5-((2-(2,2-dimethylpyrrolidin-1-yl)ethyl)carbamoyl)-2-methylpyridin-3-yl)-2-(1H-pyrazolo[3,4-b]pyridin-1-yl)pyrazolo[5,1-b]thiazole-7-carboxamide

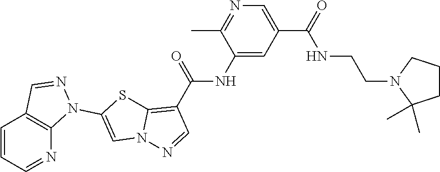 [SMILES: Cc1ncc(C(=O)NCCN2CCCC2(C)C)cc1NC(=O)c1cnn2cc(-n3ncc4cccnc43)sc12]

### Step a: methyl 5-(2-(1H-pyrazolo[3,4-b]pyridin-1-yl)pyrazolo[5,1-b]thiazole-7-carboxamido)-6-methylnicotinate

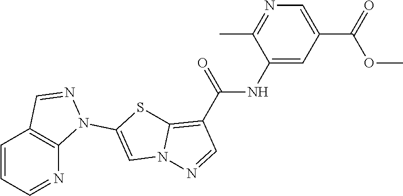 [SMILES: COC(=O)c1cnc(C)c(NC(=O)c2cnn3cc(-n4ncc5cccnc54)sc23)c1]

To a solution of methyl methyl 5-(2-bromopyrazolo[5,1-b]thiazole-7-carboxamido)-6-methylnicotinate (1 g, 2.5 mmol), 1H-pyrazolo[3,4-b]pyridine (390 mg, 3.3 mmol), CuI (140 mg, 0.74 mmol) and Cs₂CO₃ (1.2 g, 3.7 mmol) in 1,4-dioxane (10 mL) was added (1S,2S)—N1,N2-dimethylcyclohexane-1,2-diamine (210 mg, 1.5 mmol) at room temperature. The resulting mixture was stirred at 120° C. for 16 h. The reaction mixture was filtered through a pad of celite, and the pad or filter cake was washed with DCM (40 mL). The combined filtrate was concentrated under reduced pressure to give crude product, which was purified by column chromatography over silica gel (eluent: DCM/MeOH from 100:0 to 90:10) to give the crude product (260 mg, 14%) as a black solid.

### Step b: 5-(2-(1H-pyrazolo[3,4-b]pyridin-1-yl)pyrazolo[5,1-b]thiazole-7-carboxamido)-6-methylnicotinic acid

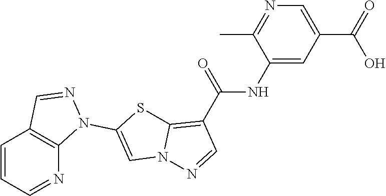 [SMILES: Cc1ncc(C(=O)O)cc1NC(=O)c1cnn2cc(-n3ncc4cccnc43)sc12]

To a solution of methyl 5-(2-(1H-pyrazolo[3,4-b]pyridin-1-yl)pyrazolo[5,1-b]thiazole-7-carboxamido)-6-methylnicotinate (260 mg, 0.6 mmol) in THE (5.4 mL) and H₂O (1.2 mL) was added LiOH H₂O (75 mg, 1.8 mmol) room temperature, The mixture was stirred at room temperature for 1 hour. The reaction mixture was poured into 10 mL of ice-water carefully and acidified with 1N HCl to pH=5. The mixture was filtered, rinsed with H₂O (10 mL). The filter cake was dried in vacuo to afford 5-(2-(1H-pyrazolo[3,4-b]pyridin-1-yl)pyrazolo[5,1-b]thiazole-7-carboxamido)-6-methylnicotinic acid (150 mg, 36%) as a brown solid.

### Step c: N-(5-((2-(2,2-dimethylpyrrolidin-1-yl)ethyl)carbamoyl)-2-methylpyridin-3-yl)-2-(1H-pyrazolo[3,4-b]pyridin-1-yl)pyrazolo[5,1-b]thiazole-7-carboxamide

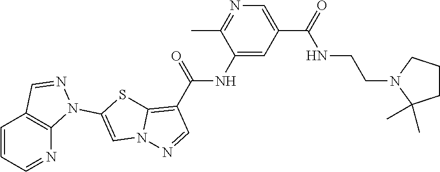 [SMILES: Cc1ncc(C(=O)NCCN2CCCC2(C)C)cc1NC(=O)c1cnn2cc(-n3ncc4cccnc43)sc12]

To a solution of 5-(2-(1H-pyrazolo[3,4-b]pyridin-1-yl)pyrazolo[5,1-b]thiazole-7-carboxamido)-6-methylnicotinic acid (130 mg, 0.19 mmol) in DMF (3 mL) was added 2-(2,2-dimethylpyrrolidin-1-yl)ethanamine (70 mg, 0.51 mmol), DIEA (0.55 mL, 3.3 mmol) and HATU (370 mg, 0.97 mmol). The mixture stirred at room temperature for 15 h and then concentrated under vacuum to give the crude product, which was purified by column chromatography over silica gel (eluent: DCM/MeOH from 100:0 to 90:10) to give the crude product. Which was purified by preparative high-performance liquid chromatography over column: Welch Xtimate C18 150*30 mm*5 um to give the title compound N-(5-((2-(2,2-dimethylpyrrolidin-1-yl)ethyl)carbamoyl)-2-methylpyridin-3-yl)-2-(1H-pyrazolo[3,4-b]pyridin-1-yl)pyrazolo[5,1-b]thiazole-7-carboxamide (40 mg, 37%) as a white solid. LCMS (ESI): mass calcd. for C₂H₂₉N₉O₂S, 543.6 m/z found, 544.3 [M+H]⁺. ¹H NMR (400 MHz, METHANOL-d₄) δ 8.93 (s, 1H), 8.80 (d, J=1.91 Hz, 1H), 8.78 (dd, J=4.65, 1.43 Hz, 1H), 8.47 (s, 1H), 8.34-8.42 (m, 3H), 7.45 (dd, J=7.99, 4.65 Hz, 1H), 3.54 (t, J=6.97 Hz, 2H), 2.92 (t, J=7.27 Hz, 2H), 2.70 (t, J=6.85 Hz, 2H), 2.65 (s, 3H), 1.79-1.91 (m, 2H), 1.68-1.76 (m, 2H), 1.09 (s, 6H).

### Example 218. 2-(1,3-Dimethyl-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide

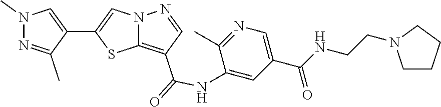 [SMILES: Cc1ncc(C(=O)NCCN2CCCC2)cc1NC(=O)c1cnn2cc(-c3cn(C)nc3C)sc12]

### Step a: 5-(2-(1,3-Dimethyl-1H-pyrazol-4-yl)pyrazolo[5,1-b]thiazole-7-carboxamido)-6-methylnicotinic acid

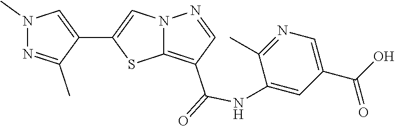 [SMILES: Cc1ncc(C(=O)O)cc1NC(=O)c1cnn2cc(-c3cn(C)nc3C)sc12]

A 2-5 mL microwave vial, equipped with a stir bar, was charged with ethyl 5-(2-bromopyrazolo[5,1-b]thiazole-7-carboxamido)-6-methylnicotinate (205 mg, 0.501 mmol), 1,3-dimethyl-1H-pyrazole-4-boronic acid pinacol ester (167 mg, 0.754 mmol), Na₂CO₃ (86.7 mg, 0.818 mmol), and DME/Water/EtOH (7:3:2) (4 mL). The mixture was sparged with nitrogen for 3 min with rapid stirring. Bis(triphenylphosphine)palladium(II) dichloride (18.0 mg, 0.0256 mmol) was added. The vial was sealed with a cap. After 30 min of irradiation in a Biotage Initiator+microwave reactor at 140° C., the mixture was allowed to cool to room temperature, and solvent was removed. The crude mixture was dissolved in MeOH, filtered through celite, concentrated, and purified by preparative high-performance liquid chromatography over column: Waters XBridge BEH C18 5 μm to give 5-(2-(1,3-dimethyl-1H-pyrazol-4-yl)pyrazolo[5,1-b]thiazole-7-carboxamido)-6-methylnicotinic acid as a white solid (13 mg, 6%). LCMS (ESI): mass calcd for C₁₈H₁₆N₆O₃S, 396.1; m/z found, 397.0 [M+H]+.

### Step b: 2-(1,3-Dimethyl-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide

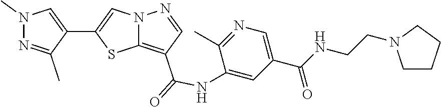 [SMILES: Cc1ncc(C(=O)NCCN2CCCC2)cc1NC(=O)c1cnn2cc(-c3cn(C)nc3C)sc12]

A 20 mL vial, equipped with a stir bar, was charged with 5-(2-(1,3-dimethyl-1H-pyrazol-4-yl)pyrazolo[5,1-b]thiazole-7-carboxamido)-6-methylnicotinic acid (12.8 mg, 0.0324 mmol), DMF (1 mL), DIEA (30 μL, 0.17 mmol), 1-(2-aminoethyl)pyrrolidine (10 μL, 0.075 mmol), and HATU (18.1 mg, 0.0476 mmol) at 23° C. After 31 h, the mixture was transferred to a 30 mL vial. Water (3 mL) was added. Solvent was removed under reduced pressure. The crude residue was purified by preparative high-performance liquid chromatography over column: Waters XBridge BEH C18 5 μm to afford 2-(1,3-dimethyl-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide as a white solid (3.0 mg, 18%). LCMS (ESI): mass calcd for C₂₄H₂₈N₈O₂S, 492.2; m/z found, 493.1 [M+H]+. ¹H NMR (METHANOL-d4, 400 MHz) δ 8.80 (d, 1H, J=2.4 Hz), 8.42 (s, 1H), 8.32 (d, 1H, J=2.0 Hz), 8.11 (s, 1H), 7.92 (s, 1H), 3.87 (s, 3H), 3.59 (t, 2H, J=6.6 Hz), 2.77 (t, 2H, J=6.8 Hz), 2.6-2.7 (m, 4H), 2.61 (s, 3H), 2.42 (s, 3H), 1.8-1.9 (m, 4H).

### Example 219. 2-(1-Methyl-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide

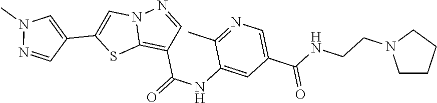 [SMILES: Cc1ncc(C(=O)NCCN2CCCC2)cc1NC(=O)c1cnn2cc(-c3cnn(C)c3)sc12]

A 30 mL vial, equipped with a stir bar, was charged with 6-methyl-5-(2-(1-methyl-1H-pyrazol-4-yl)pyrazolo[5,1-b]thiazole-7-carboxamido)nicotinic acid (65.7 mg, 0.172 mmol), 1-(2-aminoethyl)pyrrolidine (40 μL, 0.30 mmol), DMF (3 mL), and N,N-diisopropylethylamine (240 μL, 1.38 mmol). HATU (71.9 mg, 0.189 mmol) was added at 22° C. An argon atmosphere was established. After 25 h, 1-(2-aminoethyl)pyrrolidine (40 μL, 0.30 mmol), N,N-diisopropylethylamine (50 μL, 0.29 mmol), and HATU (34.5 mg, 0.0907 mmol) were added. After 22.5 h, the reaction was quenched with water (2 mL). Solvent was removed under reduced pressure. The crude residue was purified by preparative high-performance liquid chromatography over column: Waters XBridge BEH C18 5 μm to give 2-(1-methyl-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide as a white solid (47 mg, 58%). LCMS (ESI): mass calcd for C₂₃H₂₆N₈O₂S, 478.2; m/z found, 479.1 [M+H]+. ¹H NMR (CHLOROFORM-d, 400 MHz) δ 8.64 (d, 1H, J=2.0 Hz), 8.36 (d, 1H, J=2.0 Hz), 8.21 (s, 1H), 7.80 (s, 1H), 7.68 (s, 1H), 7.60 (s, 1H), 6.99 (br t, 1H, J=4.6 Hz), 3.96 (s, 3H), 3.55 (q, 2H, J=5.9 Hz), 2.70 (t, 2H, J=6.1 Hz), 2.5-2.6 (m, 7H), 1.8-1.8 (m, 4H).

### Example 220. 2-(1,5-Dimethyl-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide

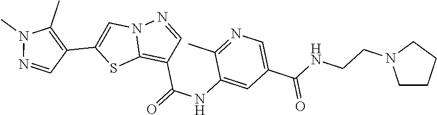 [SMILES: Cc1ncc(C(=O)NCCN2CCCC2)cc1NC(=O)c1cnn2cc(-c3cnn(C)c3C)sc12]

### Step a: Ethyl 2-(1,5-dim ethyl-1H-pyrazol-4-yl)pyrazolo[5,1-b]thiazole-7-carboxylate

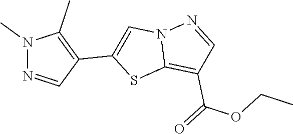 [SMILES: CCOC(=O)c1cnn2cc(-c3cnn(C)c3C)sc12]

Ethyl 2-bromopyrazolo[5,1-b]thiazole-7-carboxylate (335 mg, 1.22 mmol), 1,5-dimethyl-4-(4,4,5,5-tetramethyl-1,3,2-dioxaborolan-2-yl)-1H-pyrazole (418 mg, 1.84 mmol), cesium carbonate (678 mg, 2.08 mmol), and 1,4-dioxane:distilled water (5:1) (4 mL) were combined in a 2-5 mL microwave vial, equipped with a stir bar. The contents were sparged with nitrogen with vigorous stirring. 1,1′-Bis(diphenylphosphino)ferrocene-palladium(II)dichloride dichloromethane complex (102 mg, 0.124 mmol) was added. The vial was sealed with a cap. The reaction was irradiated in a Biotage Initiator+microwave reactor at 130° C. for 1 h and allowed to cool to room temperature. The reaction was diluted with DCM. The crude mixture was filtered through celite using MeOH. The intermediate was purified using silica gel column chromatography with a 100% Heptane to 100% EtOAc gradient to provide ethyl 2-(1,5-dimethyl-1H-pyrazol-4-yl)pyrazolo[5,1-b]thiazole-7-carboxylate as a grey solid. LCMS (ESI): mass calcd for C₁₃H₁₄N₄O₂S, 290.1; m/z found, 291.1 [M+H]+.

### Step b: 2-(1,5-Dimethyl-1H-pyrazol-4-yl)pyrazolo[5,1-b]thiazole-7-carboxylic acid

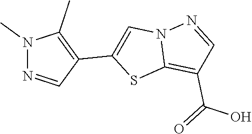 [SMILES: Cc1c(-c2cn3ncc(C(=O)O)c3s2)cnn1C]

A 30 mL vial, containing ethyl 2-(1,5-dimethyl-1H-pyrazol-4-yl)pyrazolo[5,1-b]thiazole-7-carboxylate, was charged with a stir bar, 1,4-dioxane, water (1.5 mL), and LiOH (66.6 mg, 2.78 mmol). The vial was heated at 60° C. for 41 h. The vial was allowed to cool to 23° C. The reaction was acidified to pH=1 with 1N HCl. The solvent was removed under reduced pressure. The vial was placed under high vacuum overnight to furnish 2-(1,5-dimethyl-1H-pyrazol-4-yl)pyrazolo[5,1-b]thiazole-7-carboxylic acid as a light purple solid (105 mg, 33%). LCMS (ESI): mass calcd for C_{II}H₁₀N₄O₂S, 262.1; m/z found, 263.0 [M+H]+. ¹H NMR (DMSO-d6, 400 MHz) δ 12.72 (br s, 1H), 8.47 (s, 1H), 8.16 (s, 1H), 7.71 (s, 1H), 3.80 (s, 3H), 2.43 (s, 3H).

### Step c: Ethyl 5-(2-(1,5-dimethyl-1H-pyrazol-4-yl)pyrazolo[5,1-b]thiazole-7-carboxamido)-6-methylnicotinate

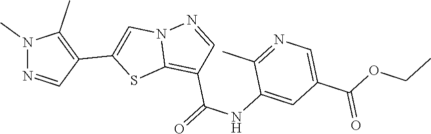 [SMILES: CCOC(=O)c1cnc(C)c(NC(=O)c2cnn3cc(-c4cnn(C)c4C)sc23)c1]

A 2-dram vial, equipped with a stir bar, was charged with 2-(1,5-dimethyl-1H-pyrazol-4-yl)pyrazolo[5,1-b]thiazole-7-carboxylic acid (52.9 mg, 0.202 mmol) and 5-amino-6-methyl-3-pyridinecarboxylic acid ethyl ester (59.6 mg, 0.321 mmol). An argon atmosphere was established. Pyridine (3 mL) and 1-(3-dimethylaminopropyl)-3-ethylcarbodiimide hydrochloride (75.0 mg, 0.391 mmol) were added. The reaction proceeded at 22° C. After 17 h, solvent was removed under reduced pressure. The crude residue was purified using silica gel column chromatography using 100% DCM to 10% MeOH/DCM gradient to give ethyl 5-(2-(1,5-dimethyl-1H-pyrazol-4-yl)pyrazolo[5,1-b]thiazole-7-carboxamido)-6-methylnicotinate as an off-white solid. LCMS (ESI): mass calcd for C₂₀H₂₀N₆O₃S, 424.1; m/z found, 425.1 [M+H]+.

### Step c: 5-(2-(1,5-Dimethyl-1H-pyrazol-4-yl)pyrazolo[5,1-b]thiazole-7-carboxamido)-6-methylnicotinic acid

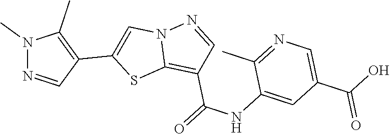 [SMILES: Cc1ncc(C(=O)O)cc1NC(=O)c1cnn2cc(-c3cnn(C)c3C)sc12]

A 30 mL vial, containing ethyl 5-(2-(1,5-dimethyl-1H-pyrazol-4-yl)pyrazolo[5,1-b]thiazole-7-carboxamido)-6-methylnicotinate, was charged with THE (2.5 mL) and NaOH (3M in H₂O) (0.3 mL, 0.9 mmol). A nitrogen atmosphere was established. The vial was heated at 45° C. for 1 h 10 min. The vial was allowed to cool to 23° C. The reaction was acidified to pH=1 with 1N HCl. The solvent was removed under reduced pressure. The vial was placed under high vacuum overnight to provide 5-(2-(1,5-dimethyl-1H-pyrazol-4-yl)pyrazolo[5,1-b]thiazole-7-carboxamido)-6-methylnicotinic acid as a white solid. LCMS (ESI): mass calcd for C₁₈H₁₆N₆O₃S, 396.1; m/z found, 397.1 [M+H]+.

### Step d: 2-(1,5-Dimethyl-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide

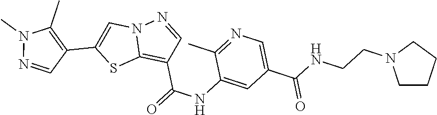 [SMILES: Cc1ncc(C(=O)NCCN2CCCC2)cc1NC(=O)c1cnn2cc(-c3cnn(C)c3C)sc12]

A 30 mL vial, containing 5-(2-(1,5-dimethyl-1H-pyrazol-4-yl)pyrazolo[5,1-b]thiazole-7-carboxamido)-6-methylnicotinic acid, was charged with a stir bar, DMF (3 mL), 1-(2-aminoethyl)pyrrolidine (50 μL, 0.38 mmol) and N,N-diisopropylethylamine (350 μL, 2.0 mmol). HATU (86.2 mg, 0.227 mmol) was added. An argon atmosphere was established. The reaction proceeded at 23° C. After 24 h, N,N-diisopropylethylamine (50 μL, 0.29), 1-(2-aminoethyl)pyrrolidine (50 μL, 0.38 mmol), and HATU (54.1 mg, 0.142 mmol) were added. After 21 h, N,N-diisopropylethylamine (200 μL, 1.15 mmol), 1-(2-aminoethyl)pyrrolidine (100 μL, 0.750 mmol), and HATU (62.0 mg, 0.163 mmol) were added. After 24 h, the reaction was quenched with water (3 mL). The water was removed under reduced pressure. The crude residue was purified by preparative high-performance liquid chromatography over column: Waters XBridge BEH C18 5 μm to afford 2-(1,5-dimethyl-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide as a white solid (19 mg, 19%). LCMS (ESI): mass calcd for C₂₄H₂₈N₈O₂S, 492.2; m/z found, 493.1 [M+H]+. ¹H NMR (CHLOROFORM-d, 400 MHz) δ 8.72 (d, 1H, J=2.0 Hz), 8.59 (d, 1H, J=2.0 Hz), 8.12 (s, 1H), 7.74 (s, 1H), 7.59 (s, 1H), 6.86 (br t, 1H, J=4.6 Hz), 3.87 (s, 3H), 3.56 (q, 2H, J=5.9 Hz), 2.71 (t, 2H, J=6.1 Hz), 2.64 (s, 3H), 2.5-2.6 (m, 4H), 2.44 (s, 3H), 1.8-1.9 (m, 4H).

### Example 221. 2-(1-(3,4-Dihydroxybutyl)-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide

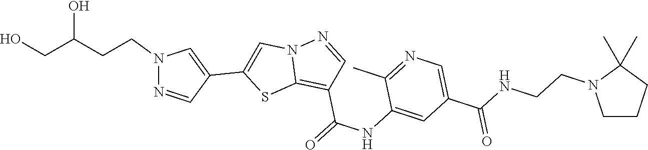 [SMILES: Cc1ncc(C(=O)NCCN2CCCC2(C)C)cc1NC(=O)c1cnn2cc(-c3cnn(CCC(O)CO)c3)sc12]

### Step a: 4-Bromo-1-(2-(2,2-dimethyl-1,3-dioxolan-4-yl)ethyl)-1H-pyrazole

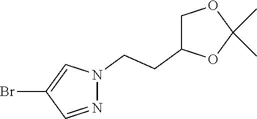

A 30 mL vial, equipped with a stir bar, was charged with 2-(2,2-dimethyl-1,3-dioxolan-4-yl)ethanol (1.0 mL, 6.9 mmol). An argon atmosphere was established. DMF (15 mL) and 2,6-dimethylpyridine (3.49 mL, 29.0 mmol) were added. The vial was cooled to 4° C. for 12 min. Methanesulfonyl chloride (0.7 mL, 9 mmol) was added. The vial was allowed to warm to 23° C. After 24 h, the mixture was diluted with EtOAc (40 mL), and brine (30 mL) was added. The organic layer was washed with cold 1M sodium sulfate (50 mL), dilute sodium bicarbonate (40 mL), and brine (40 mL). The organic extract was dried over anhydrous magnesium sulfate, filtered, and concentrated to supply 2-(2,2-dimethyl-1,3-dioxolan-4-yl)ethyl methanesulfonate as an orange oil. A 30 mL vial, containing 2-(2,2-dimethyl-1,3-dioxolan-4-yl)ethyl methanesulfonate, was charged with a stir bar, DMF (8 mL), 4-bromo-1H-pyrazole (1.03 g, 6.87 mmol), and cesium carbonate (2.72 g, 8.33 mmol). An argon atmosphere was established. The mixture was heated at 65° C. After 17 h, heating was stopped. The reaction was allowed to cool to 23° C., quenched with water (90 mL), and diluted with EtOAc (40 mL). The mixture was washed with brine (30 mL). The organic layer was dried over anhydrous magnesium sulfate, filtered, and concentrated for purification. The residue was purified using silica gel column chromatography with a 100% Heptane to 100% (3% MeOH/EtOAc) gradient to yield 4-bromo-1-(2-(2,2-dimethyl-1,3-dioxolan-4-yl)ethyl)-1H-pyrazole as a clear oil (877 mg, 41%). LCMS (ESI): mass calcd for C₁₀H₁₅BrN₂O₂, 274.0; m/z found, 275.0 [M+H]+.

### Step b: 1-(2-(2,2-Dimethyl-1,3-dioxolan-4-yl)ethyl)-4-(4,4,5,5-tetramethyl-1,3,2-dioxaborolan-2-yl)-1H-pyrazole

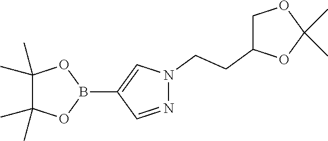

A 20 mL vial, equipped with a stir bar, was charged with 4-bromo-1-(2-(2,2-dimethyl-1,3-dioxolan-4-yl)ethyl)-1H-pyrazole (358 mg, 1.15 mmol), bis(pinacolato)diboron (332 mg, 1.28 mmol), 1,4-dioxane (4 mL), potassium acetate (235 mg, 2.37 mmol), and 1,1′-bis(diphenylphosphino)ferrocene-palladium(II)dichloride dichloromethane complex (96.4 mg, 0.118 mmol). An argon atmosphere was established. The reaction was heated at 95° C. After 8.5 h, the reaction was allowed to cool to 23° C. The mixture was concentrated. The residue was dissolved in DCM. The organic phase was washed with brine (30 mL), dried over anhydrous sodium sulfate, filtered, and concentrated for purification. The crude residue was purified using silica gel column chromatography with a 100% Heptane to 50% EtOAc/Heptane gradient to give 1-(2-(2,2-dimethyl-1,3-dioxolan-4-yl)ethyl)-4-(4,4,5,5-tetramethyl-1,3,2-dioxaborolan-2-yl)-1H-pyrazole (128 mg, 22%). LCMS (ESI): mass calcd for C₁₆H₂₇BN₂O₄, 322.2; m/z found, 323.2 [M+H]+.

### Step c: 2-(1-(3,4-Dihydroxybutyl)-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide

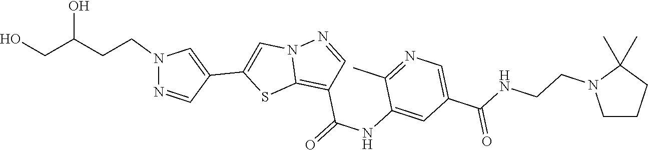 [SMILES: Cc1ncc(C(=O)NCCN2CCCC2(C)C)cc1NC(=O)c1cnn2cc(-c3cnn(CCC(O)CO)c3)sc12]

Bromo-N-(5-((2-(2,2-dimethylpyrrolidin-1-yl)ethyl)carbamoyl)-2-methylpyridin-3-yl)pyrazolo[5,1-b]thiazole-7-carboxamide (24.0 mg, 0.0475 mmol), 1-(2-(2,2-dimethyl-1,3-dioxolan-4-yl)ethyl)-4-(4,4,5,5-tetramethyl-1,3,2-dioxaborolan-2-yl)-1H-pyrazole (38.0 mg, 0.118 mmol), cesium carbonate (54.5 mg, 0.167 mmol), and 1,4-dioxane:distilled water (5:1) (4 mL) were combined in a 2-5 mL microwave vial, equipped with a stir bar. Contents were sparged with argon with vigorous stirring. 1,1′-Bis(diphenylphosphino)ferrocene-palladium(II)dichloride dichloromethane complex (14.3 mg, 0.0175 mmol) was added. The vial was sealed with a cap. The reaction was irradiated in a Biotage Initiator+microwave reactor at 130° C. for 1 h and allowed to cool to room temperature. The reaction was diluted with DCM. The crude mixture was filtered through celite using MeOH. Solvent was removed under reduced pressure. A 30 mL vial, containing 2-(1-(2-(2,2-dimethyl-1,3-dioxolan-4-yl)ethyl)-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide, was charged with a stir bar, MeOH (5 mL), and p-toluenesulfonic acid monohydrate (80.7 mg, 0.418 mmol). An argon atmosphere was established; the reaction proceeded at room temperature. After 18 h 40 min, solvent was removed under reduced pressure. Water (3 mL) was added to the crude residue and removed in vacuo. The crude residue was purified by preparative high-performance liquid chromatography over column: Waters XBridge BEH C18 5 μm to provide 2-(1-(3,4-dihydroxybutyl)-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide as a light brown solid (15 mg, 53%). LCMS (ESI): mass calcd for C₂₈H₃₆N₈O₄S, 580.2; m/z found, 581.2 [M+H]+. ¹H NMR (METHANOL-d4, 400 MHz) δ 8.77 (d, 1H, J=2.0 Hz), 8.40 (s, 1H), 8.31 (d, 1H, J=2.0 Hz), 8.24 (s, 1H), 8.09 (s, 1H), 7.85 (s, 1H), 4.35 (dd, 2H, J=6.4, 7.8 Hz), 3.4-3.6 (m, 5H), 2.91 (br t, 2H, J=7.1 Hz), 2.69 (br t, 2H, J=6.8 Hz), 2.61 (s, 3H), 2.1-2.2 (m, 1H), 1.8-1.9 (m, 3H), 1.7-1.7 (m, 2H), 1.05 (s, 6H).

### Example 222. 2-(1-(3,4-Dihydroxybutyl)-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide

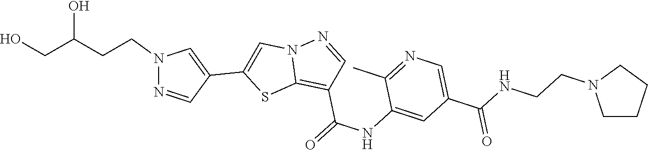 [SMILES: Cc1ncc(C(=O)NCCN2CCCC2)cc1NC(=O)c1cnn2cc(-c3cnn(CCC(O)CO)c3)sc12]

### Step a: 2-Bromo-N-(2-methyl-5-((2-(pyrrolidin-1-yl)ethyl)carbamoyl)pyridin-3-yl)pyrazolo[5,1-b]thiazole-7-carboxamide

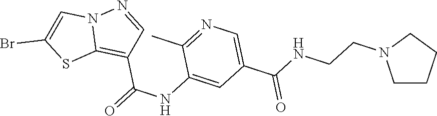 [SMILES: Cc1ncc(C(=O)NCCN2CCCC2)cc1NC(=O)c1cnn2cc(Br)sc12]

A 30 mL vial, equipped with a stir bar, was charged with 5-(2-bromopyrazolo[5,1-b]thiazole-7-carboxamido)-6-methylnicotinic acid (61.0 mg, 0.160 mmol), 1-(2-aminoethyl)pyrrolidine (50 μL, 0.38 mmol), DMF (3 mL), and DIEA (0.1 mL, 0.6 mmol). HATU (76.8 mg, 0.202 mmol) was added at 23° C. An argon atmosphere was established. After 22.5 h, the reaction was quenched with water (3 mL). Solvent was removed under reduced pressure. The crude residue was purified by preparative high-performance liquid chromatography over column: Waters XBridge BEH C18 5 μm to yield 2-bromo-N-(2-methyl-5-((2-(pyrrolidin-1-yl)ethyl)carbamoyl)pyridin-3-yl)pyrazolo[5,1-b]thiazole-7-carboxamide as a white solid (27.5 mg, 36%). LCMS (ESI): mass calcd for C₁₉H₂₁BrN₆O₂S, 476.1; m/z found, 477.0 [M+H]+. ¹H NMR (METHANOL-d4, 400 MHz) δ 8.79 (d, 1H, J=2.0 Hz), 8.47 (s, 1H), 8.34 (s, 1H), 8.30 (d, 1H, J=2.4 Hz), 3.58 (t, 2H, J=6.8 Hz), 2.75 (t, 2H, J=6.8 Hz), 2.6-2.7 (m, 4H), 2.59 (s, 3H), 1.8-1.9 (m, 4H).

### Step b: 2-(1-(3,4-Dihydroxybutyl)-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide

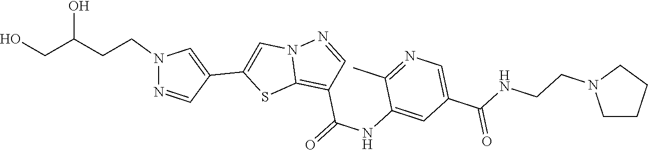 [SMILES: Cc1ncc(C(=O)NCCN2CCCC2)cc1NC(=O)c1cnn2cc(-c3cnn(CCC(O)CO)c3)sc12]

2-Bromo-N-(2-methyl-5-((2-(pyrrolidin-1-yl)ethyl)carbamoyl)pyridin-3-yl)pyrazolo[5,1-b]thiazole-7-carboxamide (27.0 mg, 0.0566 mmol) in 1.5 mL of 1,4-dioxane:distilled water (5:1), 1-(2-(2,2-dimethyl-1,3-dioxolan-4-yl)ethyl)-4-(4,4,5,5-tetramethyl-1,3,2-dioxaborolan-2-yl)-1H-pyrazole (29.3 mg, 0.0909 mmol), cesium carbonate (55.3 mg, 0.170 mmol), and 1,4-dioxane:distilled water (5:1) (1 mL), were combined in a 2-5 mL microwave vial, equipped with a stir bar. The contents were sparged with argon with vigorous stirring. 1,1′-Bis(diphenylphosphino)ferrocene-palladium(II)dichloride dichloromethane complex (9.9 mg, 0.012 mmol) was added. The vial was sealed with a cap. The mixture was irradiated in a Biotage Initiator+microwave reactor at 130° C. for 1 h and allowed to cool to room temperature.

The reaction was diluted with DCM. The crude mixture was filtered through celite using MeOH. Solvent was removed under reduced pressure. A 30 mL vial, containing 2-(1-(2-(2,2-dimethyl-1,3-dioxolan-4-yl)ethyl)-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide, was charged with a stir bar, MeOH (5 mL), and p-toluenesulfonic acid monohydrate (64.0 mg, 0.331 mmol). An argon atmosphere was established; the reaction proceeded at room temperature. After 18 h 40 min, solvent was removed under reduced pressure. Water (3 mL) was added to the crude residue and removed in vacuo. The crude residue was purified by preparative high-performance liquid chromatography over column: Waters XBridge BEH C18 5 μm to give 2-(1-(3,4-dihydroxybutyl)-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide as a light brown solid (6.8 mg, 20%). LCMS (ESI): mass calcd for C₂₆H₃₂N₈O₄S, 552.2; m/z found, 553.2 [M+H]+. ¹H NMR (METHANOL-d4, 400 MHz) δ 8.80 (d, 1H, J=2.0 Hz), 8.41 (s, 1H), 8.35 (d, 1H, J=2.0 Hz), 8.25 (s, 1H), 8.09 (s, 1H), 7.85 (s, 1H), 4.3-4.4 (m, 2H), 3.68 (t, 2H, J=6.2 Hz), 3.5-3.6 (m, 1H), 3.5-3.5 (m, 2H), 3.0-3.2 (m, 6H), 2.62 (s, 3H), 2.1-2.2 (m, 1H), 1.9-2.0 (m, 4H), 1.8-1.9 (m, 1H).

### Example 223. N-(2-methyl-5-((2-(pyrrolidin-1-yl)ethyl)carbamoyl)pyridin-3-yl)-2-(1,3,5-trimethyl-1H-pyrazol-4-yl)pyrazolo[5,1-b]thiazole-7-carboxamide

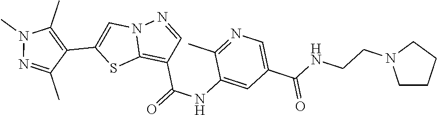 [SMILES: Cc1ncc(C(=O)NCCN2CCCC2)cc1NC(=O)c1cnn2cc(-c3c(C)nn(C)c3C)sc12]

2-Bromo-N-(2-methyl-5-((2-(pyrrolidin-1-yl)ethyl)carbamoyl)pyridin-3-yl)pyrazolo[5,1-b]thiazole-7-carboxamide (29.0 mg, 0.0607 mmol), 1,3,5-trimethyl-4-(4,4,5,5-tetramethyl-1,3,2-dioxaborolan-2-yl)-1 h-pyrazole (31.5 mg, 0.131 mmol), cesium carbonate (70.3 mg, 0.216 mmol), and 1,4-dioxane:distilled water (5:1) (2.5 mL) were combined in a 2-5 mL microwave vial, equipped with a stir bar. 1,1′-Bis(diphenylphosphino)ferrocene-palladium(II)dichloride dichloromethane complex (11.5 mg, 0.0141 mmol) was added, and nitrogen was blown over the headspace of the vial. The vial was sealed with a cap. The reaction was irradiated in a Biotage Initiator+microwave reactor at 130° C. for 1 h and allowed to cool to 23° C. The crude mixture was filtered through a cartridge with a MP-TMP metal scavenger using MeOH. The mixture was concentrated, filtered, and purified by preparative high-performance liquid chromatography over column: Waters XBridge BEH C18 5 μm to afford N-(2-methyl-5-((2-(pyrrolidin-1-yl)ethyl)carbamoyl)pyridin-3-yl)-2-(1,3,5-trimethyl-1H-pyrazol-4-yl)pyrazolo[5,1-b]thiazole-7-carboxamide as a light pink solid (28 mg, 91%). LCMS (ESI): mass calcd for C₂₅H₃₀N₈O₂S, 506.2; m/z found, 507.2 [M+H]+. ¹H NMR (CHLOROFORM-d, 400 MHz) δ 8.75 (d, 1H, J=1.7 Hz), 8.65 (s, 1H), 8.11 (s, 1H), 7.67 (s, 1H), 7.44 (br s, 1H), 6.94 (br s, 1H), 3.80 (s, 3H), 3.56 (q, 2H, J=5.9 Hz), 2.72 (t, 2H, J=6.0 Hz), 2.65 (s, 3H), 2.5-2.6 (m, 4H), 2.36 (s, 3H), 2.32 (s, 3H), 1.8-1.8 (m, 4H).

### Example 224. N-(5-((2-(2,2-dimethylpyrrolidin-1-yl)ethyl)carbamoyl)-2-methylpyridin-3-yl)-2-(3-(hydroxymethyl)-1-methyl-1H-pyrazol-4-yl)pyrazolo[5,1-b]thiazole-7-carboxamide

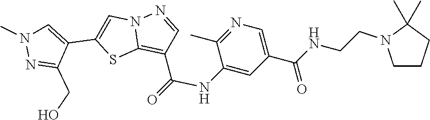 [SMILES: Cc1ncc(C(=O)NCCN2CCCC2(C)C)cc1NC(=O)c1cnn2cc(-c3cn(C)nc3CO)sc12]

A 2-Bromo-N-(5-((2-(2,2-dimethylpyrrolidin-1-yl)ethyl)carbamoyl)-2-methylpyridin-3-yl)pyrazolo[5,1-b]thiazole-6-carboxamide (29.5 mg, 0.0584 mmol), 1-methyl-4-(4,4,5,5-tetramethyl-1,3,2-dioxaborolan-2-yl)-1H-pyrazole-3-carbaldehyde (25.5 mg, 0.108 mmol), cesium carbonate (59.6 mg, 0.183 mmol), and 1,4-dioxane:distilled water (5:1) (2.5 mL), were combined in a 2-5 mL microwave vial, equipped with a stir bar. The contents were sparged with argon with vigorous stirring. 1,1′-Bis(diphenylphosphino)ferrocene-palladium(II)dichloride dichloromethane complex (13.1 mg, 0.0160 mmol) was added. The vial was sealed with a cap. The reaction was irradiated in a Biotage Initiator+microwave reactor at 130° C. for 1 h and allowed to cool to room temperature. The crude mixture was filtered through a cartridge with a MP-TMP metal scavenger using MeOH. The mixture was concentrated to give crude N-(5-((2-(2,2-dimethylpyrrolidin-1-yl)ethyl)carbamoyl)-2-methylpyridin-3-yl)-2-(3-formyl-1-methyl-1H-pyrazol-4-yl)pyrazolo[5,1-b]thiazole-6-carboxamide. LCMS (ESI): mass calcd for C₂₆H₃₀N₈O₃S, 534.2; m/z found, 534.2 [M+H]+.

A 30 mL vial, containing N-(5-((2-(2,2-dimethylpyrrolidin-1-yl)ethyl)carbamoyl)-2-methylpyridin-3-yl)-2-(3-formyl-1-methyl-1H-pyrazol-4-yl)pyrazolo[5,1-b]thiazole-6-carboxamide, was charged with a stir bar and MeOH (4 mL). An argon atmosphere was established. The vial was cooled using an ice-water bath for 10 min. Sodium borohydride (36.4 mg, 0.962 mmol) was added. After 12 h, the reaction was quenched with water (3 mL), and all solvents were removed under reduced pressure. The mixture was dissolved in MeOH, filtered, and purified by preparative high-performance liquid chromatography over column: Waters XBridge BEH C18 5 um to give N-(5-((2-(2,2-dimethylpyrrolidin-1-yl)ethyl)carbamoyl)-2-methylpyridin-3-yl)-2-(3-(hydroxymethyl)-1-methyl-1H-pyrazol-4-yl)pyrazolo[5,1-b]thiazole-7-carboxamide as a light red solid (3.5 mg, 10%). LCMS (ESI): mass calcd for C₂₆H₃₂N₈O₃S, 536.2.2; m/z found, 537.2 [M+H]+.

### Example 225. 2-(1-(2,3-Dihydroxypropyl)-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide

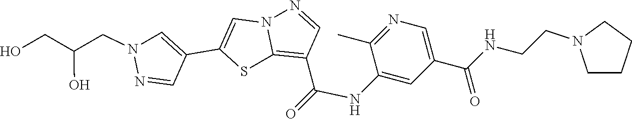 [SMILES: Cc1ncc(C(=O)NCCN2CCCC2)cc1NC(=O)c1cnn2cc(-c3cnn(CC(O)CO)c3)sc12]

### Step a: 4-Bromo-1-((2,2-dimethyl-1,3-dioxolan-4-yl)methyl)-1H-pyrazole

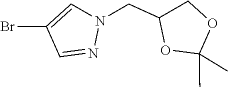

A 30 mL vial, equipped with a stir bar, was charged with 4-bromo-1H-pyrazole (1.07 g, 7.14 mmol). An argon atmosphere was established. DMF (8 mL), cesium carbonate (2.77 g, 8.49 mmol), and 4-(chloromethyl)-2,2-dimethyl-1,3-dioxolane (1.3 mL, 9.0 mmol) were added. The reaction was heated at 65° C. After 36 h, the reaction was allowed to cool to 23° C. The reaction transferred and quenched with water (80 mL). EtOAc (30 mL) was added to wash the aqueous layer. The organic layer was washed with water (40 mL), dried over anhydrous magnesium sulfate, filtered, and concentrated for silica gel chromatography. The residue was purified using silica gel column chromatography with a 100% Heptane to 100% EtOAc gradient to provide 4-bromo-1-((2,2-dimethyl-1,3-dioxolan-4-yl)methyl)-1H-pyrazole as a pale yellow oil (1.65 g, 69%). LCMS (ESI): mass calcd for C₉H₁₃BrN₂O₂, 260.0; m/z found, 261.0 [M+H]+. ¹H NMR (CHLOROFORM-d, 400 MHz) δ 7.50 (s, 1H), 7.44 (s, 1H), 4.4-4.4 (m, 1H), 4.2-4.3 (m, 2H), 4.0-4.1 (m, 1H), 3.73 (dd, 1H, J=6.1, 8.6 Hz), 1.37 (s, 3H), 1.33 (s, 3H).

### Step b: 1-((2,2-Dimethyl-1,3-dioxolan-4-yl)methyl)-4-(4,4,5,5-tetramethyl-1,3-dioxolan-2-yl)-1H-pyrazole

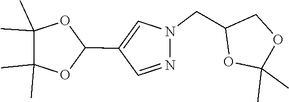

A 20 mL vial, equipped with a stir bar, was charged with 4-bromo-1-((2,2-dimethyl-1,3-dioxolan-4-yl)methyl)-1H-pyrazole (1.26 g, 4.65 mmol), bis(pinacolato)diboron (1.34 g, 5.17 mmol), 1,4-dioxane (15 mL), potassium acetate (1.08 g, 10.9 mmol), and 1,1′-bis(diphenylphosphino)ferrocene]dichloropalladium(II) (349 mg, 0.477 mmol). An argon atmosphere was established. The reaction was heated at 95° C. After 12 h, the reaction was allowed to cool to 23° C. The mixture was concentrated. The crude residue was purified using silica gel column chromatography with a 10% EtOAc/Heptane to 50% EtOAc/Heptane gradient to give 1-((2,2-dimethyl-1,3-dioxolan-4-yl)methyl)-4-(4,4,5,5-tetramethyl-1,3-dioxolan-2-yl)-1H-pyrazole as a yellow solid. LCMS (ESI): mass calcd for C₁₅H₂₅BN₂O₄, 308.2; m/z found, 309.1 [M+H]+.

### Step c: 2-(1-(2,3-Dihydroxypropyl)-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide

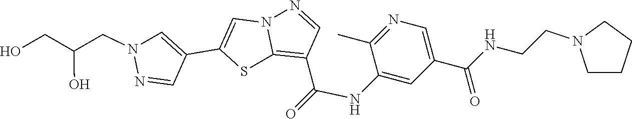 [SMILES: Cc1ncc(C(=O)NCCN2CCCC2)cc1NC(=O)c1cnn2cc(-c3cnn(CC(O)CO)c3)sc12]

2-Bromo-N-(2-methyl-5-((2-(pyrrolidin-1-yl)ethyl)carbamoyl)pyridin-3-yl)pyrazolo[5,1-b]thiazole-7-carboxamide (29.0 mg, 0.0607 mmol), 1-((2,2-dimethyl-1,3-dioxolan-4-yl)methyl)-4-(4,4,5,5-tetramethyl-1,3,2-dioxaborolan-2-yl)-1H-pyrazole (51.2 mg, 0.166 mmol), cesium carbonate (60.0 mg, 0.184 mmol), 1,1′-bis(diphenylphosphino)ferrocene-palladium(II)dichloride dichloromethane complex (10.3 mg, 0.0126 mmol), and 1,4-dioxane:distilled water (5:1) (3 mL), were combined in a 2-5 mL microwave vial, equipped with a stir bar. The contents were sparged with nitrogen with vigorous stirring. The reaction was irradiated in a Biotage Initiator+microwave reactor at 130° C. for 1 h and allowed to cool to room temperature. The crude mixture was filtered through a cartridge with MP-TMP metal scavenger using MeOH. The mixture was concentrated, filtered, and purified by preparative high-performance liquid chromatography over column: Waters XBridge BEH C18 5 μm to produce 2-(1-(2,3-dihydroxypropyl)-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide as an off-white solid (8.6 mg, 22%). LCMS (ESI): mass calcd for C₂₅1H₃N₈O₄S, 538.2; m/z found, 539.1 [M+H]+.

### Example 226. N-(5-((2-(2,2-Dimethylpyrrolidin-1-yl)ethyl)carbamoyl)-2-methylpyridin-3-yl)-2-(2-methoxypyridin-3-yl)pyrazolo[5,1-b]thiazole-7-carboxamide

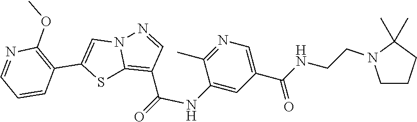 [SMILES: COc1ncccc1-c1cn2ncc(C(=O)Nc3cc(C(=O)NCCN4CCCC4(C)C)cnc3C)c2s1]

2-Bromo-N-(5-((2-(2,2-dimethylpyrrolidin-1-yl)ethyl)carbamoyl)-2-methylpyridin-3-yl)pyrazolo[5,1-b]thiazole-7-carboxamide (63.3 mg, 0.125 mmol), 2-methoxypyridine-3-boronic acid (41.7 mg, 0.259 mmol), cesium carbonate (124 mg, 0.382 mmol), and 1,4-dioxane:distilled water (5:1) (3 mL) were combined in a 2-5 mL microwave vial, equipped with a stir bar. The contents were sparged with nitrogen with vigorous stirring. 1,1′-Bis(diphenylphosphino)ferrocene-palladium(II)dichloride dichloromethane complex (10.5 mg, 0.0129 mmol) was added, and the vial was sealed with a cap. The mixture was irradiated in a Biotage Initiator+microwave reactor at 130° C. for 1 h and then two additional 30 min intervals. The mixture was allowed to cool to room temperature and filtered through Si-Tris amine metal scavenger and concentrated. The crude residue was purified by preparative high-performance liquid chromatography over column: Waters XBridge BEH C18 5 μm to afford N-(5-((2-(2,2-dimethylpyrrolidin-1-yl)ethyl)carbamoyl)-2-methylpyridin-3-yl)-2-(2-methoxypyridin-3-yl)pyrazolo[5,1-b]thiazole-7-carboxamide as a white solid (20 mg, 29%). LCMS (ESI): mass calcd for C₂₇H₃₁N₇O₃S, 533.2; m/z found, 534.2 [M+H]+. ¹H NMR (CHLOROFORM-d, 400 MHz) δ 8.70 (d, 1H, J=2.0 Hz), 8.65 (d, 1H, J=2.2 Hz), 8.47 (s, 1H), 8.20 (dd, 1H, J=1.7, 4.9 Hz), 8.15 (s, 1H), 7.80 (dd, 1H, J=1.8, 7.5 Hz), 7.55 (br s, 1H), 7.0-7.1 (m, 2H), 4.14 (s, 3H), 3.5-3.5 (m, 2H), 2.78 (t, 2H, J=7.1 Hz), 2.6-2.7 (m, 5H), 1.7-1.8 (m, 2H), 1.7-1.7 (m, 2H), 1.03 (s, 6H).

### Example 227. 2-(1-Methyl-3-(trifluoromethyl)-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide

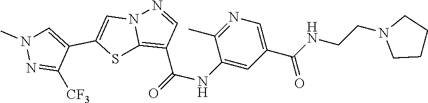 [SMILES: Cc1ncc(C(=O)NCCN2CCCC2)cc1NC(=O)c1cnn2cc(-c3cn(C)nc3C(F)(F)F)sc12]

2-Bromo-N-(2-methyl-5-((2-(pyrrolidin-1-yl)ethyl)carbamoyl)pyridin-3-yl)pyrazolo[5,1-b]thiazole-7-carboxamide (34.6 mg, 0.0725 mmol), 1-methyl-4-(4,4,5,5-tetramethyl-1,3,2-dioxaborolan-2-yl)-3-(trifluoromethyl)-1H-pyrazole (57.2 mg, 0.207 mmol), cesium carbonate (82.8 mg, 0.254 mmol), and 1,4-dioxane:distilled water (5:1) (3 mL) were combined in a 2-5 mL microwave vial, equipped with a stir bar. The contents were sparged with nitrogen with vigorous stirring. 1,1′-Bis(diphenylphosphino)ferrocene-palladium(II)dichloride dichloromethane complex (6.7 mg, 8.2 μmol) was added. The vial was sealed with a cap, and the reaction was irradiated in a Biotage Initiator+microwave reactor at 130° C. for 2 h in one-hour increments and allowed to cool to room temperature between each heating. The mixture was filtered through Si-Tris amine metal scavenger and concentrated. The crude residue was purified by preparative high-performance liquid chromatography over column: Waters XBridge BEH C18 5 μm to furnish 2-(1-methyl-3-(trifluoromethyl)-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide as a white solid (11 mg, 27%). LCMS (ESI): mass calcd for C₂₄H₂₅F₃N₈O₂S, 546.2; m/z found, 547.1 [M+H]+. ¹H NMR (CHLOROFORM-d, 400 MHz) δ 8.74 (d, 1H, J=2.0 Hz), 8.60 (d, 1H, J=2.2 Hz), 8.13 (s, 1H), 7.93 (s, 1H), 7.67 (d, 1H, J=0.7 Hz), 7.52 (br s, 1H), 6.9-7.0 (m, 1H), 4.03 (s, 3H), 3.5-3.6 (m, 2H), 2.72 (t, 2H, J=6.0 Hz), 2.64 (s, 3H), 2.5-2.6 (m, 4H), 1.8-1.8 (m, 4H).

### Example 228. 2-(2-Methoxypyridin-3-yl)-N-(2-methyl-5-((2-(2-methylpyrrolidin-1-yl)ethyl)carbamoyl)pyridin-3-yl)pyrazolo[5,1-b]thiazole-7-carboxamide

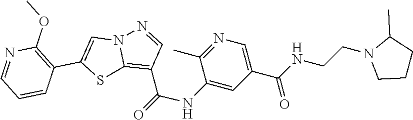 [SMILES: COc1ncccc1-c1cn2ncc(C(=O)Nc3cc(C(=O)NCCN4CCCC4C)cnc3C)c2s1]

### Step a: 2-Bromo-N-(2-methyl-5-((2-(2-methylpyrrolidin-1-yl)ethyl)carbamoyl)pyridin-3-yl)pyrazolo[5,1-b]thiazole-7-carboxamide

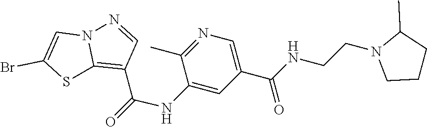 [SMILES: Cc1ncc(C(=O)NCCN2CCCC2C)cc1NC(=O)c1cnn2cc(Br)sc12]

A 100 mL round bottom flask, containing 2-(2-methylpyrrolidin-1-yl)ethan-1-amine (1.51 g, 11.7 mmol) was charged with a stir bar, DMF (15 mL), and DIEA (5.0 mL, 29 mmol). After stirring for about 5 min, 5-(2-bromopyrazolo[5,1-b]thiazole-7-carboxamido)-6-methylnicotinic acid (1.54 g, 4.05 mmol), dissolved in DMF (5 mL) and DIEA (5.0 mL, 29 mmol) was added to the reaction pot at 23° C. After 9 min, HATU (2.05 g, 5.28 mmol) was added. After 3 days, the reaction was filtered, and DMF was removed under reduced pressure. The crude residue was purified by preparative high-performance liquid chromatography over column: Waters XBridge BEH C18 5 μm to provide 2-(1-methyl-3-(trifluoromethyl)-1H-pyrazol-4-yl)-N-(2-methyl-5-((2-(pyrrolidin-1-yl)ethyl)carbamoyl)pyridin-3-yl)pyrazolo[5,1-b]thiazole-7-carboxamide as a white solid (1.13 g, 57%). LCMS (ESI): mass calcd for C₂₀H₂₃BrN₆O₂S, 490.1; m/z found, 491.1. ¹H NMR (DMSO-d6, 400 MHz) δ 10.07 (s, 1H), 8.8-8.8 (m, 2H), 8.6-8.7 (m, 2H), 8.19 (d, 1H, J=2.0 Hz), 3.4-3.5 (m, 1H), 3.3-3.4 (m, 3H), 3.1-3.2 (m, 1H), 2.9-3.0 (m, 2H), 2.3-2.4 (m, 1H), 2.1-2.3 (m, 2H), 1.8-1.9 (m, 1H), 1.6-1.7 (m, 2H), 1.2-1.3 (m, 1H), 1.01 (d, 3H, J=6.1 Hz).

### Step b: 2-(2-Methoxypyridin-3-yl)-N-(2-methyl-5-((2-(2-methylpyrrolidin-1-yl)ethyl)carbamoyl)pyridin-3-yl)pyrazolo[5,1-b]thiazole-7-carboxamide

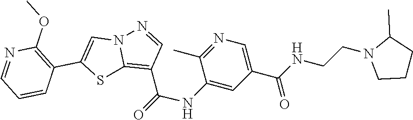 [SMILES: COc1ncccc1-c1cn2ncc(C(=O)Nc3cc(C(=O)NCCN4CCCC4C)cnc3C)c2s1]

2-Bromo-N-(2-methyl-5-((2-(2-methylpyrrolidin-1-yl)ethyl)carbamoyl)pyridin-3-yl)pyrazolo[5,1-b]thiazole-7-carboxamide (59.4 mg, 0.121 mmol), 2-methoxypyridine-3-boronic acid (42.5 mg, 0.264 mmol), cesium carbonate (120 mg, 0.369 mmol), and 1,4-dioxane:distilled water (5:1) (3 mL) were combined in a 2-5 mL microwave vial, equipped with a stir bar. The contents were sparged with nitrogen with vigorous stirring. 1,1′-Bis(diphenylphosphino)ferrocene-palladium(II)dichloride dichloromethane complex (10.4 mg, 0.0127 mmol) was added. The vial was sealed with a cap. The reaction was irradiated in a Biotage Initiator+microwave reactor at 130° C. for 1 h and allowed to cool to room temperature. The reaction was irradiated again at 130° C. for two 30 min intervals. 2-Methoxypyridine-3-boronic acid (41.0 mg, 0.255 mmol), cesium carbonate (30.1 mg, 0.0923 mmol), and 1,1′-bis(diphenylphosphino)ferrocene-palladium(II)dichloride dichloromethane complex (6.1 mg, 7.5 μmol) were added. The reaction was heated for another 1 h and allowed to cool to room temperature. The mixture was filtered through Si-Tris amine metal scavenger and concentrated. The crude residue was purified by preparative high-performance liquid chromatography over column: Waters XBridge BEH C18 5 μm to give 2-(2-methoxypyridin-3-yl)-N-(2-methyl-5-((2-(2-methylpyrrolidin-1-yl)ethyl)carbamoyl)pyridin-3-yl)pyrazolo[5,1-b]thiazole-7-carboxamide as a white solid (27.3 mg, 43%). LCMS (ESI): mass calcd for C₂₆H₂₉N₇O₃S, 519.2; m/z found, 520.1 [M+H]+. ¹H NMR (CHLOROFORM-d, 400 MHz) δ 8.72 (d, 1H, J=2.2 Hz), 8.62 (d, 1H, J=2.0 Hz), 8.47 (s, 1H), 8.20 (dd, 1H, J=1.7, 4.9 Hz), 8.16 (s, 1H), 7.80 (dd, 1H, J=1.8, 7.5 Hz), 7.60 (br s, 1H), 7.0-7.1 (m, 2H), 4.14 (s, 3H), 3.7-3.8 (m, 1H), 3.3-3.4 (m, 1H), 3.19 (dt, 1H, J=3.1, 8.6 Hz), 3.00 (ddd, 1H, J=5.5, 10.3, 12.1 Hz), 2.65 (s, 3H), 2.4-2.5 (m, 2H), 2.2-2.3 (m, 1H), 1.9-2.0 (m, 1H), 1.8-1.8 (m, 2H), 1.4-1.5 (m, 1H), 1.12 (d, 3H, J=5.9 Hz).

### Example 229. 2-(2-Methoxypyridin-3-yl)-N-(2-methyl-5-((2-(pyrrolidin-1-yl)ethyl)carbamoyl)pyridin-3-yl)pyrazolo[5,1-b]thiazole-7-carboxamide

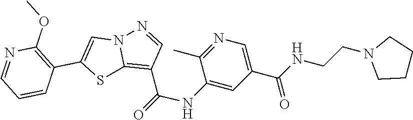 [SMILES: COc1ncccc1-c1cn2ncc(C(=O)Nc3cc(C(=O)NCCN4CCCC4)cnc3C)c2s1]

2-Bromo-N-(2-methyl-5-((2-(pyrrolidin-1-yl)ethyl)carbamoyl)pyridin-3-yl)pyrazolo[5,1-b]thiazole-7-carboxamide (61.2 mg, 0.128 mmol), 2-methoxypyridine-3-boronic acid (41.2 mg, 0.256 mmol), cesium carbonate (127 mg, 0.390 mmol), and 1,4-dioxane:distilled water (5:1) (3 mL) were combined in a 2-5 mL microwave vial, equipped with a stir bar. The contents were sparged with nitrogen with vigorous stirring. 1,1′-Bis(diphenylphosphino)ferrocene-palladium(II)dichloride dichloromethane complex (11.2 mg, 0.0137 mmol) was added. The vial was sealed with a cap. The mixture was irradiated in a Biotage Initiator+microwave reactor at 130° C. for two one-hour increments and allowed to cool to room temperature. The mixture was irradiated again at 130° C. for 30 min and allowed to cool to room temperature. Additional 2-methoxypyridine-3-boronic acid (41.2 mg, 0.256 mmol) and cesium carbonate (41.0 mg, 0.126 mmol) were added. The reaction was heated for another hour and allowed to cool to room temperature. The mixture was filtered through Si-Tris amine metal scavenger and concentrated. The crude residue was purified by preparative high-performance liquid chromatography over column: Waters XBridge BEH C18 5 μm to yield 2-(2-Methoxypyridin-3-yl)-N-(2-methyl-5-((2-(pyrrolidin-1-yl)ethyl)carbamoyl)pyridin-3-yl)pyrazolo[5,1-b]thiazole-7-carboxamide as a white solid (27.6 mg, 42%). LCMS (ESI): mass calcd for C₂₅H₂₇N₇O₃S, 505.2; m/z found, 506.1 [M+H]+. ¹H NMR (CHLOROFORM-d, 400 MHz) δ 8.75 (d, 1H, J=2.0 Hz), 8.66 (d, 1H, J=2.2 Hz), 8.48 (s, 1H), 8.21 (dd, 1H, J=1.7, 4.9 Hz), 8.12 (s, 1H), 7.81 (dd, 1H, J=1.8, 7.5 Hz), 7.44 (br s, 1H), 7.04 (dd, 1H, J=5.0, 7.5 Hz), 6.9-7.0 (m, 1H), 4.14 (s, 3H), 3.5-3.6 (m, 2H), 2.72 (t, 2H, J=6.1 Hz), 2.66 (s, 3H), 2.5-2.6 (m, 4H), 1.8-1.8 (m, 4H).

### Example 230. N-(5-((2-(2,2-dimethylpyrrolidin-1-yl)ethyl)carbamoyl)-2-methylpyridin-3-yl)-2-(5-(methylsulfonyl)pyridin-3-yl)pyrazolo[5,1-b]thiazole-7-carboxamide

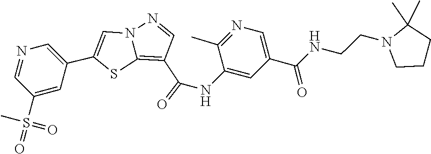 [SMILES: Cc1ncc(C(=O)NCCN2CCCC2(C)C)cc1NC(=O)c1cnn2cc(-c3cncc(S(C)(=O)=O)c3)sc12]

2-Bromo-N-(5-((2-(2,2-dimethylpyrrolidin-1-yl)ethyl)carbamoyl)-2-methylpyridin-3-yl)pyrazolo[5,1-b]thiazole-7-carboxamide (69.2 mg, 0.137 mmol), 3-(methylsulfonyl)-5-(4,4,5,5-tetramethyl-1,3,2-dioxaborolan-2-yl)pyridine (121 mg, 0.418 mmol), cesium carbonate (167 mg, 0.509 mmol), and 1,4-dioxane:distilled water (5:1) (3 mL) were combined in a 2-5 mL microwave vial, equipped with a stir bar. The contents were sparged with nitrogen with vigorous stirring. 1,1′-Bis(diphenylphosphino)ferrocene-palladium(II)dichloride dichloromethane complex (24.6 mg, 0.0301 mmol) was added. The vial was sealed with a cap. The reaction was irradiated in a Biotage Initiator+microwave reactor at 130° C. for two one-hour intervals and allowed to cool to room temperature each time. Solvent was removed under reduced pressure. The reaction contents were taken up in a minimal amount of MeOH and loaded on a neutral alumina cartridge. Alumina gel chromatography [neutral] was used to further purify the crude residue with a 100% EtOAc to 10% MeOH/EtOAc gradient. The combined fractions were concentrated, filtered, and purified by preparative high-performance liquid chromatography over column: Waters XBridge BEH C18 5 μm to give N-(5-((2-(2,2-dimethylpyrrolidin-1-yl)ethyl)carbamoyl)-2-methylpyridin-3-yl)-2-(5-(methylsulfonyl)pyridin-3-yl)pyrazolo[5,1-b]thiazole-7-carboxamide as an off-white solid (11 mg, 14%). LCMS (ESI): mass calcd for C₂₇H₃₁N₇O₄S₂, 581.2; m/z found, 582.1 [M+H]+. ¹H NMR (DMSO-d6, 400 MHz) δ 10.09 (s, 1H), 9.3-9.3 (m, 2H), 9.07 (d, 1H, J=2.2 Hz), 8.79 (d, 1H, J=2.2 Hz), 8.6-8.7 (m, 2H), 8.6-8.6 (m, 1H), 8.21 (d, 1H, J=2.2 Hz), 3.43 (s, 3H), 2.7-2.8 (m, 2H), 2.53 (s, 3H), 1.6-1.7 (m, 2H), 1.5-1.6 (m, 2H), 0.93 (s, 6H).

### Example 231. 2-(3,5-Dimethyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide

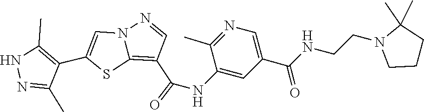 [SMILES: Cc1ncc(C(=O)NCCN2CCCC2(C)C)cc1NC(=O)c1cnn2cc(-c3c(C)n[nH]c3C)sc12]

2-Bromo-N-(5-((2-(2,2-dimethylpyrrolidin-1-yl)ethyl)carbamoyl)-2-methylpyridin-3-yl)pyrazolo[5,1-b]thiazole-7-carboxamide (97.8 mg, 0.193 mmol), tert-butyl 3,5-dimethyl-4-(4,4,5,5-tetramethyl-1,3,2-dioxaborolan-2-yl)-1H-pyrazole-1-carboxylate (168 mg, 0.523 mmol), cesium carbonate (203 mg, 0.617 mmol), 1,1′-bis(diphenylphosphino)ferrocene-palladium(II)dichloride dichloromethane complex (32.4 mg, 0.0397 mmol), and 1,4-dioxane:distilled water (5:1) (2.5 mL) were combined in a 2-5 mL microwave vial, equipped with a stir bar. The vial was sealed with a cap. The contents were sparged with nitrogen with vigorous stirring. The mixture was irradiated in a Biotage Initiator+microwave reactor at 130° C. for 1 h and allowed to cool to room temperature. Solvent was removed under reduced pressure in a 20 mL vial. The crude residue was dissolved in MeOH, and Si-Trisamine, a metal scavenger, was added. The mixture was stirred at room temperature for approximately 15.5 h. The crude mixture was filtered using a Biotage phase separator. Solvent was removed under reduced pressure. The crude residue was purified by preparative high-performance liquid chromatography over column: Waters XBridge BEH C18 5 μm to afford 2-(3,5-dimethyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide as an off-white solid (17 mg, 17%). LCMS (ESI): mass calcd for C₂₆H₃₂N₈O₂S, 520.2; m/z found, 521.2 [M+H]+. ¹H NMR (DMSO-d6, 400 MHz) δ (ppm) 12.63 (br s, 1H), 9.96 (s, 1H), 8.77 (d, 1H, J=2.0 Hz), 8.5-8.6 (m, 2H), 8.33 (s, 1H), 8.21 (d, 1H, J=2.0 Hz), 2.7-2.8 (m, 2H), 2.2-2.4 (m, 6H), 1.6-1.7 (m, 2H), 1.5-1.6 (m, 2H), 0.92 (s, 6H).

### Example 232. 2-(1-methyl-1H-pyrazol-4-yl)-N-(2-methyl-5-(2-(4-methylpiperazin-1-yl)acetamido)pyridin-3-yl)pyrazolo[5,1-b]thiazole-7-carboxamide

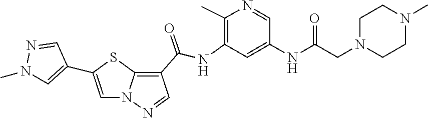

To a mixture of N-(5-amino-2-methylpyridin-3-yl)-2-(1-methyl-1H-pyrazol-4-yl)pyrazolo[5,1-b]thiazole-7-carboxamide hydrochloride (20 mg, 0.051 mmol) and 2-(4-methylpiperazin-1-yl)acetic acid (11 mg, 0.070 mmol) in pyridine (0.5 mL) was added EDCI (15 mg, 0.0782 mmol). The reaction was stirred at rt overnight. To the reaction was added CH₂Cl₂ (0.5 mL) followed by Hunig's base (0.025 mL, 0.145 mmol). The reaction was stirred at rt for another 3 h. The reaction mixture was concentrated and the residue was purified with silica gel column (24% MeOH/CH₂Cl₂ with ˜0.3% NH₄OH) to give 2-(1-methyl-1H-pyrazol-4-yl)-N-(2-methyl-5-(2-(4-methylpiperazin-1-yl)acetamido)pyridin-3-yl)pyrazolo[5,1-b]thiazole-7-carboxamide (22.1 mg, yield 87%). LCMS (ESI): mass calcd. for C₂₃H₂₇N₉O₂S, 493.20; m/z found, 494.1 [M+H]⁺. ¹H NMR (METHANOL-d4) δ: 8.67 (d, J=2.0 Hz, 1H), 8.43 (s, 1H), 8.30-8.30 (m, 1H), 8.22 (s, 1H), 8.04 (s, 1H), 7.82 (d, J=1.0 Hz, 1H), 3.95 (s, 3H), 3.35-3.53 (m, 6H), 2.79-3.08 (m, 7H), 2.52 (s, 3H).

### Example 233. N-(5-(2-(2,2-dimethylpyrrolidin-1-yl)acetamido)-2-methylpyridin-3-yl)-2-(2-methoxypyridin-3-yl)pyrazolo[5,1-b]thiazole-7-carboxamide

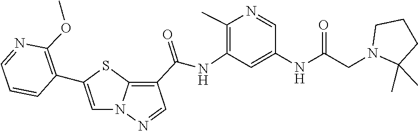 [SMILES: COc1ncccc1-c1cn2ncc(C(=O)Nc3cc(NC(=O)CN4CCCC4(C)C)cnc3C)c2s1]

A mixture of 2-(2-methoxypyridin-3-yl)pyrazolo[5,1-b]thiazole-7-carboxylic acid (105 mg, 0.381 mmol), N-(5-amino-6-methylpyridin-3-yl)-2-(2,2-dimethylpyrrolidin-1-yl)acetamide (110 mg, 0.42 mmol), and EDCI (100 mg, 0.522 mmol) in pyridine (5 mL) was heated at 70° C. for 2 h and was then kept at rt overnight. The reaction mixture was concentrated. The residue was diluted with 10% MeOH/CH₂Cl₂ and washed with aq. NaHCO₃. The aqueous layer was extracted again with 10% MeOH/CH₂Cl₂. The organic layers were combined, dried over Na₂SO₄, and concentrated. The residue was purified with silica gel column (6% MeOH/CH₂Cl₂ with ˜0.3% NH₄OH) to give N-(5-(2-(2,2-dimethylpyrrolidin-1-yl)acetamido)-2-methylpyridin-3-yl)-2-(2-methoxypyridin-3-yl)pyrazolo[5,1-b]thiazole-7-carboxamide (110 mg, yield 56%). LCMS (ESI): mass calcd. for C₂₆H₂₉N₇O₃S, 519.21; m/z found, 519.9 [M+H]⁺. ¹H NMR (CHLOROFORM-d) δ: 9.28 (br s, 1H), 8.56 (d, J=2.4 Hz, 1H), 8.43 (s, 1H), 8.37-8.41 (m, 2H), 8.31 (s, 1H), 8.17 (dd, J=4.9, 2.0 Hz, 1H), 7.98 (s, 1H), 7.75 (dd, J=7.6, 1.7 Hz, 1H), 7.00 (dd, J=7.3, 4.9 Hz, 1H), 4.11 (s, 3H), 3.21 (s, 2H), 2.87 (t, J=7.1 Hz, 2H), 2.51 (s, 3H), 1.73-1.92 (m, 4H), 1.07 (s, 6H).

### Example 234. N-(5-((2-((2-hydroxyethyl)amino)ethyl)carbamoyl)-2-methylpyridin-3-yl)-2-(1-methyl-1H-pyrazol-4-yl)pyrazolo[5,1-b]thiazole-7-carboxamide

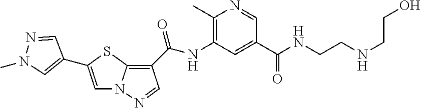 [SMILES: Cc1ncc(C(=O)NCCNCCO)cc1NC(=O)c1cnn2cc(-c3cnn(C)c3)sc12]

### Step a: tert-butyl (2-(5-amino-6-methylnicotinamido)ethyl)(2-hydroxyethyl)carbamate

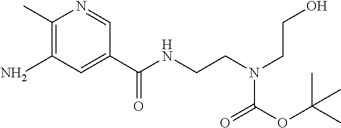 [SMILES: Cc1ncc(C(=O)NCCN(CCO)C(=O)OC(C)(C)C)cc1N]

To a mixture of 3-amino-2-methylpyridine-5-carboxylic acid (50 mg, 0.329 mmol) and tert-butyl 2-aminoethyl-2-hydroxyethylcarbamate (90 mg, 0.441 mmol) in CH₂Cl₂ (4 mL) was added HATU (200 mg, 0.526 mmol) followed by Hunig's base (0.17 mL, 0.986 mmol). The reaction was stirred at rt for 22 h. To the reaction was added aq. NaHCO₃, and the resulting mixture was extracted with EtOAc (5×). The organic layer was dried over Na₂SO₄ and concentrated. The residue was purified with silica gel column (12% MeOH/CH₂Cl₂ with ˜0.3% NH₄OH) to give tert-butyl (2-(5-amino-6-methylnicotinamido)ethyl)(2-hydroxyethyl)carbamate (89 mg, yield 80%). LCMS (ESI): mass calcd. for C₁₆H₂₆N₄O₄, 338.20; m/z found, 339.1 [M+H]⁺.

### Step b: tert-butyl (2-hydroxyethyl)(2-(6-methyl-5-(2-(1-methyl-1H-pyrazol-4-yl)pyrazolo[5,1-b]thiazole-7-carboxamido)nicotinamido)ethyl)carbamate

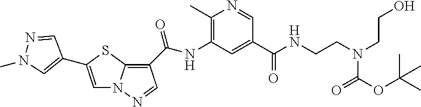 [SMILES: Cc1ncc(C(=O)NCCN(CCO)C(=O)OC(C)(C)C)cc1NC(=O)c1cnn2cc(-c3cnn(C)c3)sc12]

To a mixture of 2-(1-methyl-1H-pyrazol-4-yl)pyrazolo[5,1-b]thiazole-7-carboxylic acid (60 mg, 0.242 mmol) and tert-butyl (2-(5-amino-6-methylnicotinamido)ethyl)(2-hydroxyethyl)carbamate (89 mg, 0.263 mmol) in pyridine (2.5 mL) was added EDCI (70 mg, 0.365 mmol). The reaction was stirred at rt overnight. The reaction mixture was concentrated. The residue was diluted with EtOAc, washed with aq NaHCO₃, dried over Na₂SO₄, and concentrated. The crude product was purified with silica gel column (10% MeOH/CH₂Cl₂) to give tert-butyl (2-hydroxyethyl)(2-(6-methyl-5-(2-(1-methyl-1H-pyrazol-4-yl)pyrazolo[5,1-b]thiazole-7-carboxamido)nicotinamido)ethyl)carbamate (76 mg, yield 55%). LCMS (ESI): mass calcd. for C₂₆H₃₂N₈O₅S, 568.22; m/z found, 569.3 [M+H]⁺. ¹H NMR (CHLOROFORM-d) δ: 9.34 (br s, 1H), 9.02 (s, 1H), 8.60 (br s, 1H), 8.52 (s, 1H), 7.84 (s, 1H), 7.68-7.73 (m, 1H), 7.60-7.68 (m, 2H), 3.85-3.99 (m, 4H), 3.65-3.78 (m, 2H), 3.34-3.55 (m, 5H), 2.45-2.55 (m, 3H), 1.40-1.54 (m, 9H).

### Step c: N-(5-((2-((2-hydroxyethyl)amino)ethyl)carbamoyl)-2-methylpyridin-3-yl)-2-(1-methyl-1H-pyrazol-4-yl)pyrazolo[5,1-b]thiazole-7-carboxamide

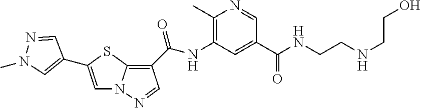 [SMILES: Cc1ncc(C(=O)NCCNCCO)cc1NC(=O)c1cnn2cc(-c3cnn(C)c3)sc12]

To a solution of tert-butyl (2-hydroxyethyl)(2-(6-methyl-5-(2-(1-methyl-1H-pyrazol-4-yl)pyrazolo[5,1-b]thiazole-7-carboxamido)nicotinamido)ethyl)carbamate (26 mg, 0.0457 mmol) in CH₂Cl₂ (1.5 mL) was added 4M HCl in dioxane (0.6 mL, 2.4 mmol). The reaction was stirred at rt for 2 h before it was concentrated. The resulting residue was washed with diethyl ether and CH₂Cl₂. The remaining solid was collected and dried to give N-(5-((2-((2-hydroxyethyl)amino)ethyl)carbamoyl)-2-methylpyridin-3-yl)-2-(1-methyl-1H-pyrazol-4-yl)pyrazolo[5,1-b]thiazole-7-carboxamide hydrochloride (23 mg, yield 93%). LCMS (ESI): δ: 9.16-9.25 (m, 1H), 9.12 (s, 1H), 8.55 (s, 1H), 8.28 (s, 1H), 8.08 (s, 1H), 7.86 (s, 1H), 3.97 (s, 3H), 3.77-3.91 (m, 4H), 3.37-3.44 (m, 2H), 3.23-3.30 (m, 2H), 2.89 (s, 3H).

### Example 235. N-(5-((2-((cyclohexylmethyl)(2-hydroxyethyl)amino)ethyl)carbamoyl)-2-methylpyridin-3-yl)-2-(1-methyl-1H-pyrazol-4-yl)pyrazolo[5,1-b]thiazole-7-carboxamide

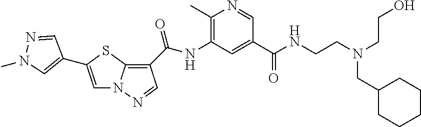 [SMILES: Cc1ncc(C(=O)NCCN(CCO)CC2CCCCC2)cc1NC(=O)c1cnn2cc(-c3cnn(C)c3)sc12]

A mixture of N-(5-((2-((2-hydroxyethyl)amino)ethyl)carbamoyl)-2-methylpyridin-3-yl)-2-(1-methyl-1H-pyrazol-4-yl)pyrazolo[5,1-b]thiazole-7-carboxamide (0.044 mmol) and cyclohexanecarbaldehyde (0.03 mL, 0.248 mmol) in MeOH (1.5 mL) and HOAc (0.15 mL) was stirred at rt for 10 min before sodium cyanoborohydride (15 mg, 0.239 mmol) was added. The reaction was stirred at rt overnight. Additional cyanoborohydride (10 mg, 0.160 mmol) was added and the reaction was stirred at rt for another 6 h. The reaction mixture was concentrated and aq. NaHCO₃ was added. The resulting mixture was extracted with EtOAc (2×). The organic layer was dried over Na₂SO₄ and concentrated. The crude product was purified with silica gel column (14% MeOH/CH₂Cl₂ with ˜0.3% NH₄OH) to give N-(5-((2-((cyclohexylmethyl)(2-hydroxyethyl)amino)ethyl)carbamoyl)-2-methylpyridin-3-yl)-2-(1-methyl-1H-pyrazol-4-yl)pyrazolo[5,1-b]thiazole-7-carboxamide (10.4 mg, yield 42%). LCMS (ESI): mass calcd. for C₂₈H₃₆N₈O₃S, 564.26; m/z found, 564.9 [M+H]⁺. ¹H NMR (METHANOL-d4) δ: 8.72 (d, J=2.4 Hz, 1H), 8.35 (s, 1H), 8.25-8.31 (m, 1H), 8.17 (s, 1H), 7.96 (s, 1H), 7.75 (s, 1H), 3.88 (s, 3H), 3.79 (br s, 2H), 3.47-3.72 (m, 2H), 3.25 (dt, J=3.3, 1.5 Hz, 4H), 2.83-3.05 (m, 2H), 2.56 (s, 3H), 1.78-1.85 (m, 2H), 1.57-1.74 (m, 3H), 1.10-1.32 (m, 4H), 0.92-1.09 (m, 2H).

### Example 236. (R)—N-(5-(((1-(2-hydroxyethyl)pyrrolidin-2-yl)methyl)carbamoyl)-2-methylpyridin-3-yl)-2-(1-methyl-1H-pyrazol-4-yl)pyrazolo[5,1-b]thiazole-7-carboxamide

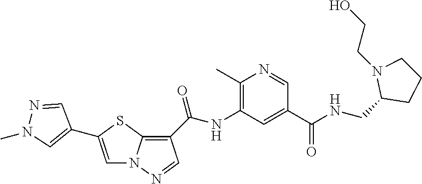 [SMILES: Cc1ncc(C(=O)NC[C@H]2CCCN2CCO)cc1NC(=O)c1cnn2cc(-c3cnn(C)c3)sc12]

### Step a: tert-butyl (R)-2-((5-amino-6-methylnicotinamido)methyl)pyrrolidine-1-carboxylate

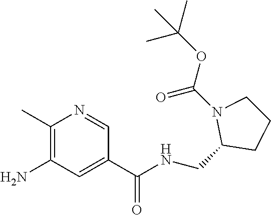 [SMILES: Cc1ncc(C(=O)NC[C@H]2CCCN2C(=O)OC(C)(C)C)cc1N]

To a mixture of 3-amino-2-methypyridine-5-carboxylic acid (100 mg, 0.657 mmol) and (R)-1-Boc-2-(aminomethyl)pyrrolidine (160 mg, 0.799 mmol) in CH₂Cl₂ (8 mL) was added Et3N (0.27 mL, 1.94 mmol) and HATU (350 mg, 0.92 mmol). The reaction was stirred at rt overnight. To the reaction was added aq. NaHCO₃ and the resulting mixture was extracted with EtOAc (2×). The organic layer was washed with aq. NaCl, dried over Na₂SO₄, and concentrated. The crude product was purified with silica gel column (5% MeOH/CH₂Cl₂) to give tert-butyl (R)-2-((5-amino-6-methylnicotinamido)methyl)pyrrolidine-1-carboxylate (158 mg, yield 72%). LCMS (ESI): mass calcd. for C₁₇H₂₆N₄O₃, 334.20; m/z found, 335.0 [M+H]⁺.

### Step b: tert-butyl (R)-2-((6-methyl-5-(2-(1-methyl-1H-pyrazol-4-yl)pyrazolo[5,1-b]thiazole-7-carboxamido)nicotinamido)methyl)pyrrolidine-1-carboxylate

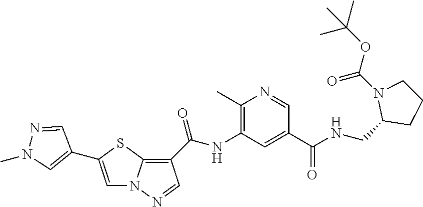 [SMILES: Cc1ncc(C(=O)NC[C@H]2CCCN2C(=O)OC(C)(C)C)cc1NC(=O)c1cnn2cc(-c3cnn(C)c3)sc12]

To a mixture of 2-(1-methyl-1H-pyrazol-4-yl)pyrazolo[5,1-b]thiazole-7-carboxylic acid (100 mg, 0.403 mmol) and tert-butyl (R)-2-((5-amino-6-methylnicotinamido)methyl)pyrrolidine-1-carboxylate (158 mg, 0.472 mmol) in pyridine (4 mL) was added EDCI (110 mg, 0.574 mmol). The reaction was stirred at rt overnight. The reaction mixture was concentrated and to the residue was added aq. NaHCO₃. The resulting mixture was extracted with EtOAc (3×). The organic layer was dried over Na₂SO₄ and concentrated. The crude product was purified with silica gel column (8% MeOH/CH₂Cl₂) to give tert-butyl (R)-2-((6-methyl-5-(2-(1-methyl-1H-pyrazol-4-yl)pyrazolo[5,1-b]thiazole-7-carboxamido)nicotinamido)methyl)pyrrolidine-1-carboxylate (110 mg, yield 48%). LCMS (ESI): mass calcd. for C₂₇H₃₂N₈O₄S, 564.23; m/z found, 564.9 [M+H]⁺.

### Step c: (R)-2-(1-methyl-1H-pyrazol-4-yl)-N-(2-methyl-5-((pyrrolidin-2-ylmethyl)carbamoyl)pyridin-3-yl)pyrazolo[5,1-b]thiazole-7-carboxamide

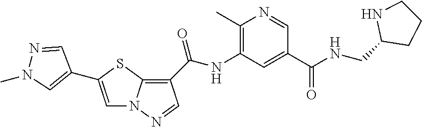 [SMILES: Cc1ncc(C(=O)NC[C@H]2CCCN2)cc1NC(=O)c1cnn2cc(-c3cnn(C)c3)sc12]

To a solution of tert-butyl (R)-2-((6-methyl-5-(2-(1-methyl-1H-pyrazol-4-yl)pyrazolo[5,1-b]thiazole-7-carboxamido)nicotinamido)methyl)pyrrolidine-1-carboxylate (110 mg, 0.195 mmol) in CH₂Cl₂ (2.5 mL) was added TFA (0.5 mL). The reaction was stirred at rt for 1.5 h. The reaction mixture was concentrated to give (R)-2-(1-methyl-1H-pyrazol-4-yl)-N-(2-methyl-5-((pyrrolidin-2-ylmethyl)carbamoyl)pyridin-3-yl)pyrazolo[5,1-b]thiazole-7-carboxamide which will be used in next step without purification. LCMS (ESI): mass calcd. for C₂₂H₂₄N₈O₂S, 464.17; m/z found, 464.9 [M+H]⁺.

### Step d: (R)—N-(5-(((1-(2-((tert-butyldimethylsilyl)oxy)ethyl)pyrrolidin-2-yl)methyl)carbamoyl)-2-methylpyridin-3-yl)-2-(1-methyl-1H-pyrazol-4-yl)pyrazolo[5,1-b]thiazole-7-carboxamide

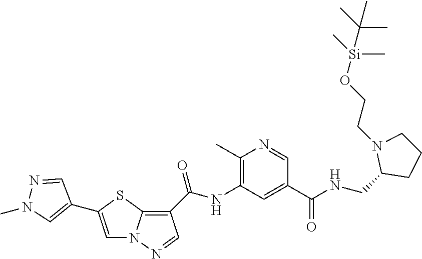 [SMILES: Cc1ncc(C(=O)NC[C@H]2CCCN2CCO[Si](C)(C)C(C)(C)C)cc1NC(=O)c1cnn2cc(-c3cnn(C)c3)sc12]

To a solution of (R)-2-(1-methyl-1H-pyrazol-4-yl)-N-(2-methyl-5-((pyrrolidin-2-ylmethyl)carbamoyl)pyridin-3-yl)pyrazolo[5,1-b]thiazole-7-carboxamide (0.195 mmol) and 2-((tert-butyldimethylsilyl)oxy)acetaldehyde (70 mg, 0.402 mmol) in HOAc (0.4 mL) and 1,2-dichloroethane (4 mL) at rt was added NaBH(OAc)₃ (80 mg, 0.377 mmol). The reaction was stirred at rt overnight. Additional 2-((tert-butyldimethylsilyl)oxy)acetaldehyde (70 mg, 0.402 mmol) and NaBH(OAc)₃ (80 mg, 0.377 mmol) was added. The reaction was stirred at rt for another 6 h. The reaction was quenched with aq. NaHCO₃ and the resulting was extracted with EtOAc (3×). The organic layer was dried over Na₂SO₄ and concentrated. The crude product was purified with silica gel column (12% MeOH/CH₂Cl₂ with ˜0.3% NH₄OH) to give (R)—N-(5-(((1-(2-((tert-butyldimethylsilyl)oxy)ethyl)pyrrolidin-2-yl)methyl)carbamoyl)-2-methylpyridin-3-yl)-2-(1-methyl-1H-pyrazol-4-yl)pyrazolo[5,1-b]thiazole-7-carboxamide (55 mg, yield 45%). LCMS (ESI): mass calcd. for C₃₀H₄₂N₈O₃SSi, 622.29; m/z found, 623.0 [M+H]⁺.

### Step e: (R)—N-(5-(((1-(2-hydroxyethyl)pyrrolidin-2-yl)methyl)carbamoyl)-2-methylpyridin-3-yl)-2-(1-methyl-1H-pyrazol-4-yl)pyrazolo[5,1-b]thiazole-7-carboxamide

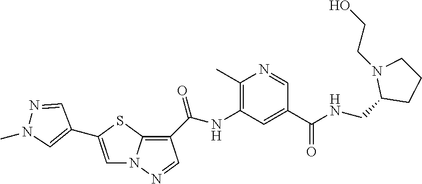 [SMILES: Cc1ncc(C(=O)NC[C@H]2CCCN2CCO)cc1NC(=O)c1cnn2cc(-c3cnn(C)c3)sc12]

To a solution of (R)—N-(5-(((1-(2-((tert-butyldimethylsilyl)oxy)ethyl)pyrrolidin-2-yl)methyl)carbamoyl)-2-methylpyridin-3-yl)-2-(1-methyl-1H-pyrazol-4-yl)pyrazolo[5,1-b]thiazole-7-carboxamide (52 mg, 0.0835 mmol) in MeOH (0.5 mL) and CH₂Cl₂ (2 mL) was added 4M HCl in dioxane (0.5 mL, 2.0 mmol). The reaction was stirred at rt for 1.5 h. The reaction mixture was concentrated, and the residue was washed with diethyl ether and CH₂Cl₂. The remaining solid was collected and dried to give (R)—N-(5-(((1-(2-hydroxyethyl)pyrrolidin-2-yl)methyl)carbamoyl)-2-methylpyridin-3-yl)-2-(1-methyl-1H-pyrazol-4-yl)pyrazolo[5,1-b]thiazole-7-carboxamide hydrochloride (45 mg, yield 93%). LCMS (ESI): mass calcd. for C₂₄H₂₈N₈O₃S, 508.20; m/z found, 508.8 [M+H]⁺. ¹H NMR (METHANOL-d4) δ: 9.13 (d, J=2.0 Hz, 1H), 9.01-9.08 (m, 1H), 8.48 (s, 1H), 8.19 (s, 1H), 8.00 (s, 1H), 7.79 (s, 1H), 3.72-3.95 (m, 9H), 3.57-3.68 (m, 1H), 3.19-3.32 (m, 2H), 2.81 (s, 3H), 2.21-2.34 (m, 1H), 1.90-2.14 (m, 3H).

### Example 237. N-(5-((1-(tert-butyl)azetidin-3-yl)carbamoyl)-2-methylpyridin-3-yl)-2-(1-methyl-1H-pyrazol-4-yl)pyrazolo[5,1-b]thiazole-7-carboxamide

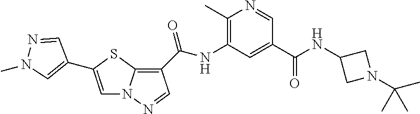 [SMILES: Cc1ncc(C(=O)NC2CN(C(C)(C)C)C2)cc1NC(=O)c1cnn2cc(-c3cnn(C)c3)sc12]

To a mixture of 6-methyl-5-(2-(1-methyl-1H-pyrazol-4-yl)pyrazolo[5,1-b]thiazole-7-carboxamido)nicotinic acid (41 mg, 0.107 mmol) and 1-(tert-butyl)azetidin-3-amine (18 mg, 0.14 mmol) in DMF (1 mL) was added Hunig's base (0.055 mL, 0.319 mmol) followed by HATU (65 mg, 0.171 mmol). The reaction was stirred at rt overnight. The reaction mixture was diluted with 10% MeOH/CH₂Cl₂ and washed with aq. NaHCO₃. The aqueous layer was extracted with 10% MeOH/CH₂Cl₂. The organic layers were combined, dried over Na₂SO₄, and concentrated. The crude product was purified with silica gel column (14% MeOH/CH₂Cl₂ with 0.3˜0.4% NH₄OH) to give N-(5-((1-(tert-butyl)azetidin-3-yl)carbamoyl)-2-methylpyridin-3-yl)-2-(1-methyl-1H-pyrazol-4-yl)pyrazolo[5,1-b]thiazole-7-carboxamide (40.6 mg, yield 77%). LCMS (ESI): mass calcd. for C₂₄H₂₈N₈O₂S, 492.21; m/z found, 492.9 [M+H]⁺. ¹H NMR (METHANOL-d4) δ: 8.80 (d, J=2.0 Hz, 1H), 8.37-8.46 (m, 1H), 8.35 (d, J=2.0 Hz, 1H), 8.23 (s, 1H), 8.04 (s, 1H), 7.83 (s, 1H), 4.68 (t, J=7.6 Hz, 1H), 3.96 (s, 3H), 3.88 (br t, J=8.6 Hz, 2H), 3.63-3.82 (m, 2H), 2.63 (s, 3H), 1.18 (s, 9H).

### Example 238. N-(5-((1-(tert-butyl)azetidin-3-yl)carbamoyl)-2-methylpyridin-3-yl)-2-(1-methyl-1H-indazol-4-yl)pyrazolo[5,1-b]thiazole-7-carboxamide

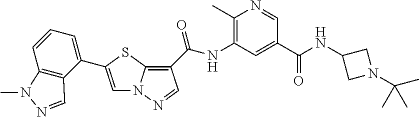 [SMILES: Cc1ncc(C(=O)NC2CN(C(C)(C)C)C2)cc1NC(=O)c1cnn2cc(-c3cccc4c3cnn4C)sc12]

### Example 239. JNJ-86964202 N-(5-(2-((tert-butylamino)methyl)aziridine-1-carbonyl)-2-methylpyridin-3-yl)-2-(1-methyl-1H-indazol-4-yl)pyrazolo[5,1-b]thiazole-7-carboxamide

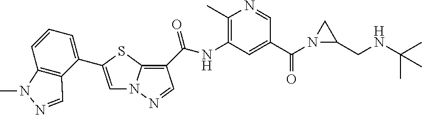 [SMILES: Cc1ncc(C(=O)N2CC2CNC(C)(C)C)cc1NC(=O)c1cnn2cc(-c3cccc4c3cnn4C)sc12]

### Step a: 2-bromo-N-(5-((1-(tert-butyl)azetidin-3-yl)carbamoyl)-2-methylpyridin-3-yl)pyrazolo[5,1-b]thiazole-7-carboxamide

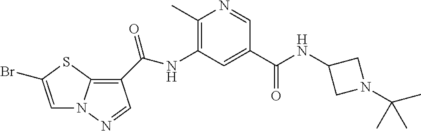 [SMILES: Cc1ncc(C(=O)NC2CN(C(C)(C)C)C2)cc1NC(=O)c1cnn2cc(Br)sc12]

To a suspension of 5-(2-bromopyrazolo[5,1-b]thiazole-7-carboxamido)-6-methylnicotinic acid (50 mg, 0.131 mmol) and 1-(tert-butyl)azetidin-3-amine (20 mg, 0.156 mmol) in CH₂Cl₂ (2 mL) was added Hunig's base (0.1 mL, 0.58 mmol) followed 1-propanephosphonic anhydride (50% solution in EtOAc) (0.16 mL, 0.269 mmol). The reaction was stirred at rt overnight, Additional 1-(tert-butyl)azetidin-3-amine (10 mg, 0.078 mmol) and 1-propanephosphonic anhydride (50% solution in EtOAc) (0.08 mL, 0.135 mmol) was added. The reaction was stirred for another 2.5 h. The reaction was diluted with 10% MeOH/CH₂Cl₂ and washed with aq. NaHCO₃. The aqueous layer was extracted with 10% MeOH/CH₂Cl₂ (2×). The organic layers were combined, dried over Na₂SO₄, and concentrated. The crude product was purified with silica gel column (12% MeOH/CH₂Cl₂ with 0.3% NH₄OH) to give 2-bromo-N-(5-((1-(tert-butyl)azetidin-3-yl)carbamoyl)-2-methylpyridin-3-yl)pyrazolo[5,1-b]thiazole-7-carboxamide (45 mg, yield 70%). LCMS (ESI): mass calcd. for C₂₀H₂₃BrN₆O₂S, 490.08; m/z found, 490.8 [M+H]⁺.

### Step b: N-(5-((1-(tert-butyl)azetidin-3-yl)carbamoyl)-2-methylpyridin-3-yl)-2-(1-methyl-1H-indazol-4-yl)pyrazolo[5,1-b]thiazole-7-carboxamide

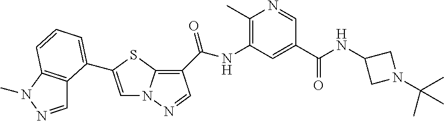 [SMILES: Cc1ncc(C(=O)NC2CN(C(C)(C)C)C2)cc1NC(=O)c1cnn2cc(-c3cccc4c3cnn4C)sc12]

### N-(5-(2-((tert-butylamino)methyl)aziridine-1-carbonyl)-2-methylpyridin-3-yl)-2-(1-methyl-1H-indazol-4-yl)pyrazolo[5,1-b]thiazole-7-carboxamide

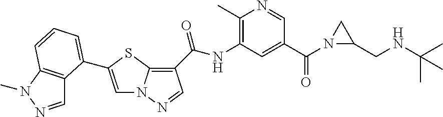 [SMILES: Cc1ncc(C(=O)N2CC2CNC(C)(C)C)cc1NC(=O)c1cnn2cc(-c3cccc4c3cnn4C)sc12]

A mixture of 2-bromo-N-(5-((1-(tert-butyl)azetidin-3-yl)carbamoyl)-2-methylpyridin-3-yl)pyrazolo[5,1-b]thiazole-7-carboxamide (45 mg, 0.0916 mmol), 1-methyl-4-(4,4,5,5-tetramethyl-1,3,2-dioxaborolan-2-yl)-1H-indazole (35 mg, 0.136 mmol), PdCl₂(dppf) (7 mg, 0.0096 mmol), and Cs₂CO₃ (60 mg, 0.184 mmol) in 1,4-dioxane (1 mL) and H₂O (0.2 mL) under argon was heated at 130° C. by microwave for 1 h. Additional PdCl₂(dppf) (4 mg) was added and the reaction was heated at 130° C. for another 1 h. The reaction mixture was diluted with 10% MeOH/CH₂Cl₂ and filtered. The solution was concentrated and the crude product was purified with silica gel column (12-14% MeOH/CH₂Cl₂ with 0.3˜0.4% NH₄OH) to give N-(5-(2-((tert-butylamino)methyl)aziridine-1-carbonyl)-2-methylpyridin-3-yl)-2-(1-methyl-1H-indazol-4-yl)pyrazolo[5,1-b]thiazole-7-carboxamide (10.2 mg, yield 21%). LCMS (ESI): mass calcd. for C₂₈H₃₀N₈O₂S, 542.22; m/z found, 542.8 [M+H]⁺. ¹H NMR (METHANOL-d4) δ: 8.84 (d, J=2.0 Hz, 1H), 8.50-8.58 (m, 1H), 8.49 (s, 1H), 8.41 (d, J=2.0 Hz, 1H), 8.37 (d, J=1.0 Hz, 1H), 7.56-7.68 (m, 1H), 7.51 (dd, J=8.3, 6.8 Hz, 1H), 7.43 (d, J=6.8 Hz, 1H), 4.54-4.71 (m, 1H), 4.36-4.48 (m, 1H), 4.19-4.33 (m, 1H), 4.14 (s, 3H), 2.75-2.85 (m, 2H), 2.64 (s, 3H), 1.16 (s, 9H).

This was followed by N-(5-((1-(tert-butyl)azetidin-3-yl)carbamoyl)-2-methylpyridin-3-yl)-2-(1-methyl-1H-indazol-4-yl)pyrazolo[5,1-b]thiazole-7-carboxamide (9.7 mg, yield 20%). LCMS (ESI): mass calcd. for C₂₈H₃₀N₈O₂S, 542.22; m/z found, 542.8 [M+H]⁺. ¹H NMR (METHANOL-d4) δ: 8.79 (d, J=2.0 Hz, 1H), 8.44-8.59 (m, 2H), 8.37 (s, 1H), 8.31 (d, J=2.4 Hz, 1H), 7.62 (d, J=8.3 Hz, 1H), 7.48-7.56 (m, 1H), 7.38-7.48 (m, 1H), 4.60 (t, J=6.8 Hz, 1H), 4.15 (s, 3H), 3.50-3.71 (m, 2H), 3.27-3.38 (m, 2H), 2.64 (s, 3H), 1.05 (s, 9H).

### Example 240. N-(5-((2-(2,2-dimethylpyrrolidin-1-yl)ethyl)carbamoyl)-2-methylpyridin-3-yl)-2-(1-methyl-1H-indazol-4-yl)pyrazolo[5,1-b]thiazole-7-carboxamide

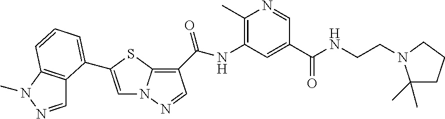 [SMILES: Cc1ncc(C(=O)NCCN2CCCC2(C)C)cc1NC(=O)c1cnn2cc(-c3cccc4c3cnn4C)sc12]

### Step a: 6-methyl-5-(2-(1-methyl-1H-indazol-4-yl)pyrazolo[5,1-b]thiazole-7-carboxamido)nicotinic acid

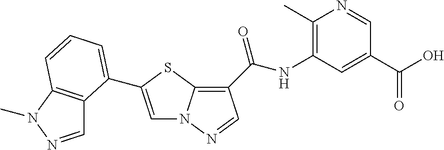 [SMILES: Cc1ncc(C(=O)O)cc1NC(=O)c1cnn2cc(-c3cccc4c3cnn4C)sc12]

A mixture of methyl 5-(2-bromopyrazolo[5,1-b]thiazole-7-carboxamido)-6-methylnicotinate (200 mg, 0.506 mmol), 1-methyl-4-(4,4,5,5-tetramethyl-1,3,2-dioxaborolan-2-yl)-1H-indazole (165 mg, 0.639 mmol), Cs₂CO₃ (350 mg, 1.074 mmol), and PdCl₂(dppf) (36 mg, 0.0492 mmol) in 1,4-dioxane (4 mL) and H₂O (0.8 mL) was heated at 130° C. by microwave under argon for 1 h. Additional PdCl₂(dppf) (18 mg, 0.0246 mmol) was added and the reaction was heated at 130° C. for another 1 h. The reaction mixture was diluted with CH₂Cl₂ and filtered. The solid was diluted with H₂O and the pH of the aqueous solution was adjusted to ˜3 with 2N HCl. The resulting mixture was filtered and the solid was collected and washed with 20% MeOH/CH₂Cl₂. The solution was concentrated to give crude 6-methyl-5-(2-(1-methyl-1H-indazol-4-yl)pyrazolo[5,1-b]thiazole-7-carboxamido)nicotinic acid (50 mg) which will be used in next step without further purification.

### Step b: N-(5-((2-(2,2-dimethylpyrrolidin-1-yl)ethyl)carbamoyl)-2-methylpyridin-3-yl)-2-(1-methyl-1H-indazol-4-yl)pyrazolo[5,1-b]thiazole-7-carboxamide

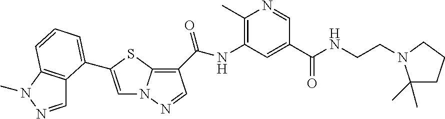 [SMILES: Cc1ncc(C(=O)NCCN2CCCC2(C)C)cc1NC(=O)c1cnn2cc(-c3cccc4c3cnn4C)sc12]

To a mixture of 6-methyl-5-(2-(1-methyl-1H-indazol-4-yl)pyrazolo[5,1-b]thiazole-7-carboxamido)nicotinic acid (50 mg, 0.116 mmol) and 2-(2,2-dimethylpyrrolidin-1-yl)ethan-1-amine (20 mg, 0.141 mmol) in DMF (1 mL) was added Hunig's base (0.07 mL, 0.406 mmol) followed by HATU (70 mg, 0.184 mmol). The reaction was stirred at rt overnight. The reaction was diluted with 10% MeOH/CH₂Cl₂ and washed with aq. NaHCO₃. The aqueous layer was extracted again with 10% MeOH/CH₂Cl₂. The organic layers were combined, dried over Na₂SO₄ and concentrated. The crude product was purified with silica gel column (12% MeOH/CH₂Cl₂ with ˜0.3% NH₄OH). The purified N-(5-((2-(2,2-dimethylpyrrolidin-1-yl)ethyl)carbamoyl)-2-methylpyridin-3-yl)-2-(1-methyl-1H-indazol-4-yl)pyrazolo[5,1-b]thiazole-7-carboxamide was washed with MeOH (1 mL). The remaining solid was collected and dried to give pure N-(5-((2-(2,2-dimethylpyrrolidin-1-yl)ethyl)carbamoyl)-2-methylpyridin-3-yl)-2-(1-methyl-1H-indazol-4-yl)pyrazolo[5,1-b]thiazole-7-carboxamide (22.1 mg, yield 34%). LCMS (ESI): mass calcd. for C₂₉H₃₂N₈O₂S, 556.24; m/z found, 556.8 [M+H]⁺. ¹H NMR (METHANOL-d4) δ: 8.80 (d, J=2.0 Hz, 1H), 8.47-8.55 (m, 1H), 8.43-8.47 (m, 1H), 8.31-8.40 (m, 2H), 7.58-7.62 (m, 1H), 7.49-7.57 (m, 1H), 7.42 (d, J=6.8 Hz, 1H), 4.16 (s, 3H), 3.39-4.08 (m, 5H), 3.10-3.30 (m, 1H), 2.60-2.72 (m, 3H), 1.96-2.30 (m, 4H), 1.24-1.63 (m, 6H).

### Example 241. N-(5-(3-(2,2-dimethylpyrrolidin-1-yl)propanamido)-2-methylpyridin-3-yl)-2-(1-methyl-1H-indazol-4-yl)pyrazolo[5,1-b]thiazole-7-carboxamide

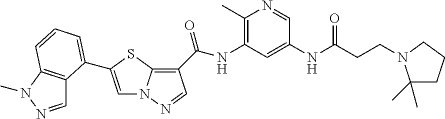 [SMILES: Cc1ncc(NC(=O)CCN2CCCC2(C)C)cc1NC(=O)c1cnn2cc(-c3cccc4c3cnn4C)sc12]

### Step a: tert-butyl (6-methyl-5-(2-(1-methyl-1H-indazol-4-yl)pyrazolo[5,1-b]thiazole-7-carboxamido)pyridin-3-yl)carbamate

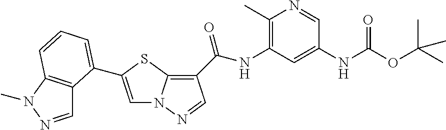 [SMILES: Cc1ncc(NC(=O)OC(C)(C)C)cc1NC(=O)c1cnn2cc(-c3cccc4c3cnn4C)sc12]

To a mixture of 2-(1-methyl-1H-indazol-4-yl)pyrazolo[5,1-b]thiazole-7-carboxylic acid (137 mg, 0.459 mmol) and tert-butyl (5-amino-6-methylpyridin-3-yl)carbamate (130 mg, 0.582 mmol) in pyridine (5 mL) was added EDCI (140 mg, 0.73 mmol). The reaction was stirred at 60° C. for 16 h. The reaction mixture was concentrated, and the residue was diluted with EtOAc and aq NaHCO₃. The resulting mixture was filtered. The solid was washed with H₂O and EtOAc, and dried to give tert-butyl (6-methyl-5-(2-(1-methyl-1H-indazol-4-yl)pyrazolo[5,1-b]thiazole-7-carboxamido)pyridin-3-yl)carbamate (148 mg, yield 64%). LCMS (ESI): mass calcd. for C₂₅H₂₅N₇O₃S, 503.17; m/z found, 503.9 [M+H]⁺.

### Step b: N-(5-amino-2-methylpyridin-3-yl)-2-(1-methyl-1H-indazol-4-yl)pyrazolo[5,1-b]thiazole-7-carboxamide

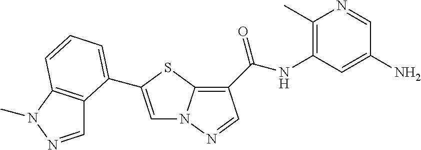 [SMILES: Cc1ncc(N)cc1NC(=O)c1cnn2cc(-c3cccc4c3cnn4C)sc12]

A mixture of tert-butyl (6-methyl-5-(2-(1-methyl-1H-indazol-4-yl)pyrazolo[5,1-b]thiazole-7-carboxamido)pyridin-3-yl)carbamate (148 mg, 0.294 mmol) and HCl (4M in dioxane) (1 mL, 4 mmol) in CH₂Cl₂ (3 mL) was stirred at rt for 5 h. The reaction mixture was diluted with diethyl ether and filtered. The solid was washed with diethyl ether and dried to give N-(5-amino-2-methylpyridin-3-yl)-2-(1-methyl-1H-indazol-4-yl)pyrazolo[5,1-b]thiazole-7-carboxamide hydrochloride, which will be used in next step without further purification. LCMS (ESI): mass calcd. for C₂₀H₁₇N₇OS, 403.12; m/z found, 403.8 [M+H]⁺.

### Step c: N-(5-acrylamido-2-methylpyridin-3-yl)-2-(1-methyl-1H-indazol-4-yl)pyrazolo[5,1-b]thiazole-7-carboxamide

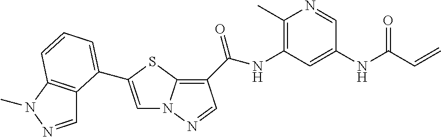 [SMILES: C=CC(=O)Nc1cnc(C)c(NC(=O)c2cnn3cc(-c4cccc5c4cnn5C)sc23)c1]

To a suspension of N-(5-amino-2-methylpyridin-3-yl)-2-(1-methyl-1H-indazol-4-yl)pyrazolo[5,1-b]thiazole-7-carboxamide hydrochloride (90 mg, 0.205 mmol) and Et₃N (0.11 mL, 0.791 mmol) in CH₂Cl₂ (3 mL) at 0° C. was added 3-chloropropanoyl chloride (0.035 mL, 0.367 mmol). The reaction was warmed to rt and stirred overnight. The reaction mixture was concentrated and the residue was suspended in H₂O and filtered. The solid was dried to give crude N-(5-acrylamido-2-methylpyridin-3-yl)-2-(1-methyl-1H-indazol-4-yl)pyrazolo[5,1-b]thiazole-7-carboxamide (80 mg), which will be used in next step without purification. LCMS (ESI): mass calcd. for C₂₃H₁₉N₇O₂S, 457.13; m/z found, 457.8 [M+H]⁺.

### Step d: N-(5-(3-(2,2-dimethylpyrrolidin-1-yl)propanamido)-2-methylpyridin-3-yl)-2-(1-methyl-1H-indazol-4-yl)pyrazolo[5,1-b]thiazole-7-carboxamide

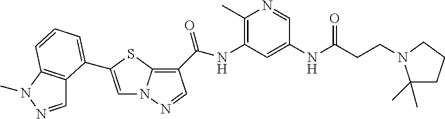 [SMILES: Cc1ncc(NC(=O)CCN2CCCC2(C)C)cc1NC(=O)c1cnn2cc(-c3cccc4c3cnn4C)sc12]

A solution of N-(5-acrylamido-2-methylpyridin-3-yl)-2-(1-methyl-1H-indazol-4-yl)pyrazolo[5,1-b]thiazole-7-carboxamide (30 mg, 0.0656 mmol), 2,2-dimethylpyrrolidine (15 mg, 0.151 mmol), and Et₃N (0.03 mL, 0.216 mmol) in ¹PrOH (1 mL) was heated at 100° C. by microwave for 5 h. The reaction mixture was concentrated. The residue was purified with silica gel column (10% MeOH/CH₂Cl₂ with ˜0.5% NH₄OH) to give N-(5-(3-(2,2-dimethylpyrrolidin-1-yl)propanamido)-2-methylpyridin-3-yl)-2-(1-methyl-1H-indazol-4-yl)pyrazolo[5,1-b]thiazole-7-carboxamide (15.4 mg, yield 42%). LCMS (ESI): mass calcd. for C₂₉H₃₂N₈O₂S, 556.24; m/z found, 556.8 [M+H]⁺. ¹H NMR (METHANOL-d4) δ: 8.44-8.51 (m, 2H), 8.40 (s, 1H), 8.34 (s, 1H), 8.18-8.31 (m, 1H), 7.58 (d, J=6.4 Hz, 1H), 7.49-7.55 (m, 1H), 7.34-7.49 (m, 1H), 4.16 (s, 3H), 2.85 (dt, J=19.2, 6.8 Hz, 4H), 2.55-2.60 (m, 2H), 2.53 (s, 3H), 1.74-1.93 (m, 4H), 1.11 (s, 6H).

### Example 242. (R)-2-(2-methoxypyridin-3-yl)-N-(2-methyl-5-((2-(2-methylpyrrolidin-1-yl)ethyl)carbamoyl)pyridin-3-yl)pyrazolo[5,1-b]thiazole-7-carboxamide

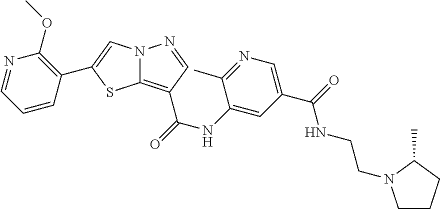 [SMILES: COc1ncccc1-c1cn2ncc(C(=O)Nc3cc(C(=O)NCCN4CCC[C@H]4C)cnc3C)c2s1]

### Step a: (R)-2-bromo-N-(2-methyl-5-((2-(2-methylpyrrolidin-1-yl)ethyl)carbamoyl)pyridin-3-yl)pyrazolo[5,1-b]thiazole-7-carboxamide

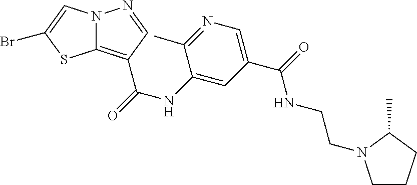 [SMILES: Cc1ncc(C(=O)NCCN2CCC[C@H]2C)cc1NC(=O)c1cnn2cc(Br)sc12]

A 30 mL vial, equipped with a stir bar, was charged with (R)-2-methylpyrrolidine (0.969 g, 11.0 mmol), DMF (10 mL), K₂CO₃ (3.07 g, 22.2 mmol), and tert-butyl 2-bromoethylcarbamate (2.74 g, 12.0 mmol). A nitrogen atmosphere was established. The reaction proceeded at 23° C. After 8 days, the reaction was quenched with water (80 mL). Extract was obtained using EtOAc (3×20 mL). The organic phase was concentrated under reduced pressure to form tert-butyl (R)-(2-(2-methylpyrrolidin-1-yl)ethyl)carbamate. A 30 mL vial, containing tert-butyl (R)-(2-(2-methylpyrrolidin-1-yl)ethyl)carbamate, was charged with a stir bar, hydrogen chloride solution in 4.0M in dioxane (20.0 mL, 80.2 mmol). A nitrogen atmosphere was established. The reaction proceeded at 23° C. overnight. Solvent was removed to form the HCl salt of (R)-2-(2-methylpyrrolidin-1-yl)ethan-1-amine.

A 250 mL round bottom flask, containing (R)-2-(2-methylpyrrolidin-1-yl)ethan-1-amine (1.42 g, 11.0 mmol), was charged with a stir bar, DMF (20 mL) and N,N-diisopropylethylamine (6.0 mL, 34 mmol). The mixture was stirred for 5 min. 5-(2-Bromopyrazolo[5,1-b]thiazole-7-carboxamido)-6-methylnicotinic acid (2.48 g, 6.51 mmol) in DMF (20 mL) was added to the reaction pot. HATU (3.22 g, 8.30 mmol) was added. The reaction was placed under a nitrogen atmosphere. The reaction proceeded at 21° C. After approximately 6 d 18 h, solvent was removed under reduced pressure at 50° C. The crude residue was allowed to cool to room temperature. The residue was purified by preparative high-performance liquid chromatography over column: Waters XBridge BEH C18 5 μm to give (R)-2-bromo-N-(2-methyl-5-((2-(2-methylpyrrolidin-1-yl)ethyl)carbamoyl)pyridin-3-yl)pyrazolo[5,1-b]thiazole-7-carboxamide as a white solid (588 mg, 18%). LCMS (ESI): mass calcd for C₂₀H₂₃BrN₆O₂S, 490.1; m/z found, 491.0 [M+H]+.

### Step b: (R)-2-(2-methoxypyridin-3-yl)-N-(2-methyl-5-((2-(2-methylpyrrolidin-1-yl)ethyl)carbamoyl)pyridin-3-yl)pyrazolo[5,1-b]thiazole-7-carboxamide

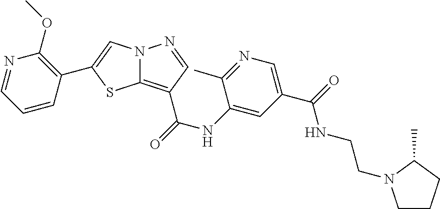 [SMILES: COc1ncccc1-c1cn2ncc(C(=O)Nc3cc(C(=O)NCCN4CCC[C@H]4C)cnc3C)c2s1]

(R)-2-bromo-N-(2-methyl-5-((2-(2-methylpyrrolidin-1-yl)ethyl)carbamoyl)pyridin-3-yl)pyrazolo[5,1-b]thiazole-7-carboxamide (128 mg, 0.260 mmol), 2-methoxypyridine-3-boronic acid pinacol ester (160 mg, 0.667 mmol), cesium carbonate (348 mg, 1.06 mmol), 1,1′-bis(diphenylphosphino)ferrocene-palladium(II)dichloride dichloromethane complex (38.8 mg, 0.0475 mmol), and 1,4-dioxane:distilled water (5:1) (3 mL) were combined in a 2-5 mL microwave vial, equipped with a stir bar. The vial was sealed with a cap. The contents were sparged with nitrogen with vigorous stirring. The mixture was irradiated in a Biotage Initiator+microwave reactor at 130° C. for 1 h and allowed to cool to room temperature. Solvent was removed under reduced pressure in a 20 mL vial. The crude residue was dissolved in MeOH. Si-Trisamine was added. The mixture was stirred at room temperature for approximately 15.5 h. The crude mixture was filtered using a Biotage phase separator. Solvent was removed under reduced pressure. The residue was purified by preparative high-performance liquid chromatography over column: Waters XBridge BEH C18 5 μm to provide (R)-2-(2-methoxypyridin-3-yl)-N-(2-methyl-5-((2-(2-methylpyrrolidin-1-yl)ethyl)carbamoyl)pyridin-3-yl)pyrazolo[5,1-b]thiazole-7-carboxamide as a white solid (24 mg, 16%). LCMS (ESI): mass calcd for C₂₆H₂₉N₇O₃S, 519.2; m/z found, 520.2 [M+H]+. ¹H NMR (CHLOROFORM-d, 400 MHz) δ 8.75 (d, 1H, J=2.2 Hz), 8.67 (d, 1H, J=2.0 Hz), 8.48 (s, 1H), 8.21 (dd, 1H, J=1.7, 4.9 Hz), 8.14 (s, 1H), 7.81 (dd, 1H, J=1.7, 7.6 Hz), 7.5-7.6 (m, 1H), 7.19 (br s, 1H), 7.03 (dd, 1H, J=4.9, 7.6 Hz), 4.14 (s, 3H), 3.7-3.8 (m, 1H), 3.3-3.5 (m, 1H), 3.2-3.3 (m, 1H), 3.0-3.1 (m, 1H), 2.66 (s, 3H), 2.4-2.6 (m, 2H), 2.2-2.3 (m, 1H), 1.9-2.0 (m, 1H), 1.8-1.9 (m, 2H), 1.4-1.5 (m, 1H), 1.15 (d, 3H, J=6.1 Hz).

### Example 243. (S)-(1-methylpyrrolidin-2-yl)methyl (6-methyl-5-(2-(1-methyl-1H-pyrazol-4-yl)pyrazolo[5,1-b]thiazole-7-carboxamido)pyridin-3-yl)carbamate

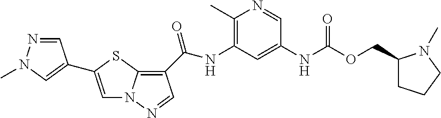 [SMILES: Cc1ncc(NC(=O)OC[C@@H]2CCCN2C)cc1NC(=O)c1cnn2cc(-c3cnn(C)c3)sc12]

To a mixture of N-(5-amino-2-methylpyridin-3-yl)-2-(1-methyl-1H-pyrazol-4-yl)pyrazolo[5,1-b]thiazole-7-carboxamide hydrochloride (30 mg, 0.0769 mmol) in DMF (0.5 mL) was added Et₃N (0.06 mL, 0.432 mmol) followed by CDI (30 mg, 0.185 mmol). The reaction was stirred at rt for 1.5 h. (S)-(1-methylpyrrolidin-2-yl)methanol (45 mg, 0.391 mmol) was then added and the reaction was heated at 80° C. by microwave for 1 h. To the reaction mixture was added aq. NaHCO₃ and the resulting mixture was extracted with 10% MeOH/CH₂Cl₂ (3×). The organic layer was dried over Na₂SO₄ and concentrated. The crude product was purified with silica gel column (15% MeOH/CH₂Cl₂ with 0.3-0.4% NH₄OH) to give (S)-(1-methylpyrrolidin-2-yl)methyl (6-methyl-5-(2-(1-methyl-1H-pyrazol-4-yl)pyrazolo[5,1-b]thiazole-7-carboxamido)pyridin-3-yl)carbamate (16.6 mg, yield 44%). LCMS (ESI): mass calcd. for C₂₃H₂₆N₈O₃S, 494.18; m/z found, 494.9 [M+H]⁺. ¹H NMR (METHANOL-d4) δ: 8.41-8.45 (m, 1H), 8.38 (s, 1H), 8.16-8.25 (m, 1H), 8.13 (d, J=2.0 Hz, 1H), 8.01 (s, 1H), 7.82 (s, 1H), 4.05-4.26 (m, 2H), 3.95 (s, 3H), 3.04-3.15 (m, 1H), 2.55-2.74 (m, 1H), 2.46-2.51 (m, 6H), 2.31-2.41 (m, 1H), 1.98-2.13 (m, 1H), 1.75-1.86 (m, 2H), 1.61-1.75 (m, 1H).

### Example 244. (R)-(1-methylpyrrolidin-2-yl)methyl (6-methyl-5-(2-(1-methyl-11H-pyrazol-4-yl)pyrazolo[5,1-b]thiazole-7-carboxamido)pyridin-3-yl)carbamate

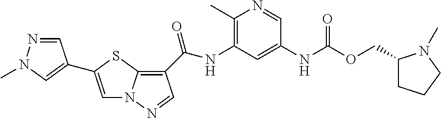 [SMILES: Cc1ncc(NC(=O)OC[C@H]2CCCN2C)cc1NC(=O)c1cnn2cc(-c3cnn(C)c3)sc12]

To a mixture of N-(5-amino-2-methylpyridin-3-yl)-2-(1-methyl-1H-pyrazol-4-yl)pyrazolo[5,1-b]thiazole-7-carboxamide hydrochloride (40 mg, 0.103 mmol) in DMF (0.7 mL) was added Et₃N (0.08 mL, 0.576 mmol) followed by CDI (35 mg, 0.216 mmol). The reaction was stirred at for 2 h. (R)-(1-methylpyrrolidin-2-yl)methanol (60 mg, 0.521 mmol) was then added and the reaction was heated at 80° C. by microwave for 1 h. To the reaction mixture was added aq. NaHCO₃ and the resulting mixture was extracted with 10% MeOH/CH₂Cl₂ (3×). The organic layer was dried over Na₂SO₄ and concentrated. The crude product was purified with silica gel column (14% MeOH/CH₂Cl₂ with 0.3-0.4% NH₄OH) to give (R)-(1-methylpyrrolidin-2-yl)methyl (6-methyl-5-(2-(1-methyl-1H-pyrazol-4-yl)pyrazolo[5,1-b]thiazole-7-carboxamido)pyridin-3-yl)carbamate (24.5 mg, yield 48%). LCMS (ESI): mass calcd. for C₂₃H₂₆N₈O₃S, 494.18; m/z found, 494.9 [M+H]⁺. ¹H NMR (METHANOL-d4) δ: 8.41-8.46 (m, 1H), 8.39 (s, 1H), 8.21-8.21 (m, 1H), 8.13 (d, J=2.4 Hz, 1H), 8.02 (s, 1H), 7.82 (s, 1H), 4.16-4.30 (m, 2H), 3.95 (s, 3H), 3.07-3.25 (m, 1H), 2.73-2.80 (m, 1H), 2.55 (s, 3H), 2.49 (s, 3H), 2.38-2.48 (m, 1H), 1.98-2.20 (m, 1H), 1.65-1.92 (m, 3H)

### Example 245. (S)-(1-isopropylpyrrolidin-2-yl)methyl (6-methyl-5-(2-(1-methyl-11H-pyrazol-4-yl)pyrazolo[5,1-b]thiazole-7-carboxamido)pyridin-3-yl)carbamate

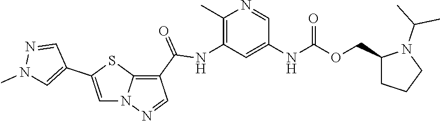 [SMILES: Cc1ncc(NC(=O)OC[C@@H]2CCCN2C(C)C)cc1NC(=O)c1cnn2cc(-c3cnn(C)c3)sc12]

To a mixture of N-(5-amino-2-methylpyridin-3-yl)-2-(1-methyl-1H-pyrazol-4-yl)pyrazolo[5,1-b]thiazole-7-carboxamide hydrochloride (40 mg, 0.0938 mmol) in DMF (0.7 mL) was added Et₃N (0.07 mL, 0.504 mmol) followed by CDI (35 mg, 0.216 mmol). The reaction was stirred at for 2 h. (S)-(1-isopropylpyrrolidin-2-yl)methanol (60 mg, 0.419 mmol) was then added and the reaction was heated at 80° C. by microwave for 1 h. To the reaction mixture was added aq. NaHCO₃ and the resulting mixture was extracted with 10% MeOH/CH₂Cl₂ (2×). The organic layer was dried over Na₂SO₄ and concentrated. The crude product was purified with silica gel column (12% MeOH/CH₂Cl₂ with ˜0.3% NH₄OH) to give (S)-(1-isopropylpyrrolidin-2-yl)methyl (6-methyl-5-(2-(1-methyl-1H-pyrazol-4-yl)pyrazolo[5,1-b]thiazole-7-carboxamido)pyridin-3-yl)carbamate (30.8 mg, yield 63%). LCMS (ESI): mass calcd. for C₂₅H₃₀N₈O₃S, 522.22; m/z found, 522.9 [M+H]⁺. ¹H NMR (METHANOL-d4) δ: 8.39-8.42 (m, 1H), 8.38 (s, 1H), 8.17-8.21 (m, 1H), 8.14 (d, J=2.0 Hz, 1H), 8.00 (s, 1H), 7.81 (s, 1H), 4.17 (dd, J=10.8, 4.9 Hz, 1H), 3.88-3.97 (m, 4H), 3.14 (dq, J=7.9, 4.0 Hz, 1H), 2.88-3.05 (m, 2H), 2.53-2.64 (m, 1H), 2.48 (s, 3H), 1.71-1.93 (m, 4H), 1.16 (d, J=6.8 Hz, 3H), 1.01-1.14 (m, 3H).

### Example 246. 2-(pyrrolidin-1-yl)ethyl (6-methyl-5-(2-(1-methyl-1H-pyrazol-4-yl)pyrazolo[5,1-b]thiazole-7-carboxamido)pyridin-3-yl)carbamate

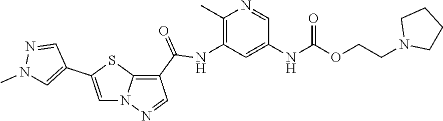 [SMILES: Cc1ncc(NC(=O)OCCN2CCCC2)cc1NC(=O)c1cnn2cc(-c3cnn(C)c3)sc12]

To a mixture of N-(5-amino-2-methylpyridin-3-yl)-2-(1-methyl-1H-pyrazol-4-yl)pyrazolo[5,1-b]thiazole-7-carboxamide hydrochloride (40 mg, 0.103 mmol) in DMF (0.7 mL) was added Et₃N (0.08 mL, 0.576 mmol) followed by CDI (40 mg, 0.247 mmol). The reaction was at rt stirred for 1.5 h. 2-(pyrrolidin-1-yl)ethan-1-ol (60 mg, 0.521 mmol) was then added and the reaction was heated at 80° C. by microwave for 1 h. To the reaction mixture was added aq. NaHCO₃ and the resulting mixture was extracted with 10% MeOH/CH₂Cl₂ (3×). The organic layer was dried over Na₂SO₄ and concentrated. The crude product was purified with silica gel column (12-14% MeOH/CH₂Cl₂ with 0.3-0.4% NH₄OH) to give 2-(pyrrolidin-1-yl)ethyl (6-methyl-5-(2-(1-methyl-1H-pyrazol-4-yl)pyrazolo[5,1-b]thiazole-7-carboxamido)pyridin-3-yl)carbamate (30.1 mg, yield 59%). LCMS (ESI): mass calcd. for C₂₃H₂₆N₈O₃S, 494.18; m/z found, 494.9 [M+H]⁺. ¹H NMR (METHANOL-d4) δ: 8.40-8.43 (m, 1H), 8.38 (s, 1H), 8.17-8.22 (m, 1H), 8.13 (d, J=2.0 Hz, 1H), 8.00 (s, 1H), 7.81 (s, 1H), 4.26-4.34 (m, 2H), 3.94 (s, 3H), 2.76-2.87 (m, 2H), 2.59-2.69 (m, 4H), 2.48 (s, 3H), 1.77-1.88 (m, 4H).

### Example 247. 2-(2,2-dimethylpyrrolidin-1-yl)ethyl (6-methyl-5-(2-(1-methyl-11H-pyrazol-4-yl)pyrazolo[5,1-b]thiazole-7-carboxamido)pyridin-3-yl)carbamate

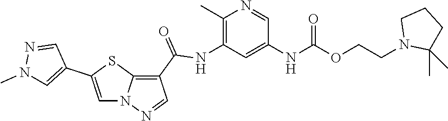

### Step a: 2-(2,2-dimethylpyrrolidin-1-yl)ethan-1-ol

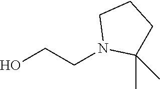

A mixture of 2,2-dimethylpyrrolidine (440 mg, 4.44 mmol), 2-bromoethan-1-ol (0.35 mL, 4.94 mmol), and K₂CO₃ (1500 mg, 10.9 mmol) in CH₃CN (12 mL) was heated to reflux for 5 h. The reaction mixture was cooled to rt, diluted with CH₂Cl₂, and filtered. The solution was concentrated and the crude product was purified with silica gel column (30% MeOH/CH₂Cl₂ with ˜1% NH₄OH) to give 2-(2,2-dimethylpyrrolidin-1-yl)ethan-1-ol (340 mg, yield 54%).

### Step b: 2-(2,2-dimethylpyrrolidin-1-yl)ethyl (6-methyl-5-(2-(1-methyl-1H-pyrazol-4-yl)pyrazolo[5,1-b]thiazole-7-carboxamido)pyridin-3-yl)carbamate

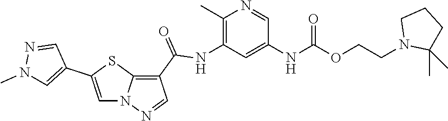

To a mixture of N-(5-amino-2-methylpyridin-3-yl)-2-(1-methyl-1H-pyrazol-4-yl)pyrazolo[5,1-b]thiazole-7-carboxamide hydrochloride (40 mg, 0.103 mmol) in DMF (0.5 mL) was added Et₃N (0.07 mL, 0.504 mmol) followed by CDI (35 mg, 0.216 mmol). The reaction was stirred at rt for 2 h. A solution of 2-(2,2-dimethylpyrrolidin-1-yl)ethan-1-ol (76 mg, 0.531 mmol) in DMF (0.3 mL) was then added and the reaction was heated at 80° C. by microwave for 1 h. To the reaction mixture was added aq. NaHCO₃ and the resulting mixture was extracted with 10% MeOH/CH₂Cl₂ (3×). The organic layer was dried over Na₂SO₄ and concentrated. The crude product was purified with silica gel column (14% MeOH/CH₂Cl₂ with ˜0.3% NH₄OH) to give 2-(2,2-dimethylpyrrolidin-1-yl)ethyl (6-methyl-5-(2-(1-methyl-1H-pyrazol-4-yl)pyrazolo[5,1-b]thiazole-7-carboxamido)pyridin-3-yl)carbamate (22.5 mg, yield 42%). LCMS (ESI): mass calcd. for C₂₅H₃₀N₈O₃S, 522.22; m/z found, 522.9 [M+H]⁺. ¹H NMR (METHANOL-d4) δ: 8.41-8.46 (m, 1H), 8.39 (s, 1H), 8.22 (s, 1H), 8.13 (d, J=2.4 Hz, 1H), 8.03 (s, 1H), 7.83 (s, 1H), 4.27 (t, J=6.1 Hz, 2H), 3.96 (s, 3H), 2.83-2.99 (m, 2H), 2.69-2.83 (m, 2H), 2.49 (s, 3H), 1.75-1.90 (m, 2H), 1.63-1.74 (m, 2H), 1.05 (s, 6H).

### Example 248. 2-(pyrrolidin-1-yl)propyl (6-methyl-5-(2-(1-methyl-1H-pyrazol-4-yl)pyrazolo[5,1-b]thiazole-7-carboxamido)pyridin-3-yl)carbamate

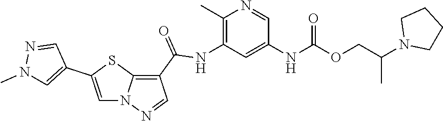 [SMILES: Cc1ncc(NC(=O)OCC(C)N2CCCC2)cc1NC(=O)c1cnn2cc(-c3cnn(C)c3)sc12]

To a mixture of N-(5-amino-2-methylpyridin-3-yl)-2-(1-methyl-1H-pyrazol-4-yl)pyrazolo[5,1-b]thiazole-7-carboxamide hydrochloride (30 mg, 0.0769 mmol) in DMF (0.7 mL) was added Et₃N (0.06 mL, 0.432 mmol) followed by CDI (25 mg, 0.154 mmol). The reaction was stirred at rt for 2 h. 2-(pyrrolidin-1-yl)propan-1-ol (45 mg, 0.348 mmol) was then added and the reaction was heated at 80° C. by microwave for 1 h. To the reaction mixture was added aq. NaHCO₃ and the resulting mixture was extracted with 10% MeOH/CH₂Cl₂ (3×). The organic layer was dried over Na₂SO₄ and concentrated. The crude product was purified with silica gel column (14% MeOH/CH₂Cl₂ with 0.3-0.4% NH₄OH) to give 2-(pyrrolidin-1-yl)propyl (6-methyl-5-(2-(1-methyl-1H-pyrazol-4-yl)pyrazolo[5,1-b]thiazole-7-carboxamido)pyridin-3-yl)carbamate (9 mg, yield 23%). LCMS (ESI): mass calcd. for C₂₄H₂₈N₈O₃S, 508.20; m/z found, 508.8 [M+H]⁺. ¹H NMR (METHANOL-d4) δ: 8.42-8.46 (m, 1H), 8.36-8.42 (m, 1H), 8.18-8.27 (m, 1H), 8.14 (d, J=2.4 Hz, 1H), 8.01-8.05 (m, 1H), 7.83 (s, 1H), 4.21-4.37 (m, 2H), 3.95 (s, 3H), 2.91 (br s, 5H), 2.49 (s, 3H), 1.84-1.97 (m, 4H), 1.30 (d, J=6.8 Hz, 3H).

### Example 249. 2-methyl-2-(pyrrolidin-1-yl)propyl (6-methyl-5-(2-(1-methyl-1H-pyrazol-4-yl)pyrazolo[5,1-b]thiazole-7-carboxamido)pyridin-3-yl)carbamate

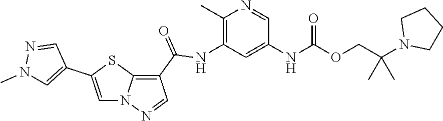 [SMILES: Cc1ncc(NC(=O)OCC(C)(C)N2CCCC2)cc1NC(=O)c1cnn2cc(-c3cnn(C)c3)sc12]

To a mixture of N-(5-amino-2-methylpyridin-3-yl)-2-(1-methyl-1H-pyrazol-4-yl)pyrazolo[5,1-b]thiazole-7-carboxamide hydrochloride (50 mg, 0.117 mmol) in DMF (0.6 mL) was added Et₃N (0.07 mL, 0.504 mmol) followed by CDI (35 mg, 0.216 mmol). The reaction was stirred at rt for 2 h. 2-methyl-2-(pyrrolidin-1-yl)propan-1-ol (65 mg, 0.454 mmol) was then added and the reaction was heated at 80° C. by microwave for 1 h. To the reaction mixture was added aq. NaHCO₃ and the resulting mixture was extracted with 10% MeOH/CH₂Cl₂ (3×). The organic layer was dried over Na₂SO₄ and concentrated. The crude product was purified with silica gel column (14% MeOH/CH₂Cl₂ with 0.3-0.4% NH₄OH) to give 2-methyl-2-(pyrrolidin-1-yl)propyl (6-methyl-5-(2-(1-methyl-1H-pyrazol-4-yl)pyrazolo[5,1-b]thiazole-7-carboxamido)pyridin-3-yl)carbamate (8.2 mg, yield 13%). LCMS (ESI): mass calcd. for C₂₅H₃₀N₈O₃S, 522.22; m/z found, 522.9 [M+H]⁺. ¹H NMR (METHANOL-d4) δ: 8.42-8.48 (m, 1H), 8.39 (s, 1H), 8.18-8.28 (m, 1H), 8.14 (d, J=2.4 Hz, 1H), 8.03 (s, 1H), 7.83 (s, 1H), 4.16 (s, 2H), 3.96 (s, 3H), 2.75-2.89 (m, 4H), 2.49 (s, 3H), 1.74-1.90 (m, 4H), 1.20 (s, 6H).

### Example 250. 2-(2-azabicyclo[2.2.1]heptan-2-yl)ethyl (6-methyl-5-(2-(1-methyl-11H-pyrazol-4-yl)pyrazolo[5,1-b]thiazole-7-carboxamido)pyridin-3-yl)carbamate

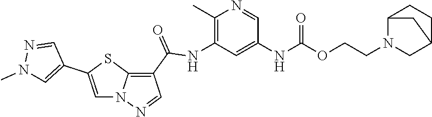 [SMILES: Cc1ncc(NC(=O)OCCN2CC3CCC2C3)cc1NC(=O)c1cnn2cc(-c3cnn(C)c3)sc12]

To a mixture of N-(5-amino-2-methylpyridin-3-yl)-2-(1-methyl-1H-pyrazol-4-yl)pyrazolo[5,1-b]thiazole-7-carboxamide hydrochloride (40 mg, 0.0938 mmol) in DMF (0.7 mL) was added Et₃N (0.07 mL, 0.504 mmol) followed by CDI (35 mg, 0.216 mmol). The reaction was at rt for 2.5 h. Additional CDI (25 mg, 0.154 mmol) was added and the reaction was stirred at rt for another 2 h. 2-(2-azabicyclo[2.2.1]heptan-2-yl)ethan-1-ol (60 mg, 0.425 mmol) was then added and the reaction was heated at 80° C. by microwave for 1 h. To the reaction mixture was added aq. NaHCO₃ and the resulting mixture was extracted with 10% MeOH/CH₂Cl₂ (2×). The organic layer was dried over Na₂SO₄ and concentrated. The crude product was purified with silica gel column (16-18% MeOH/CH₂Cl₂ with ˜0.4% NH₄OH) to give 2-(2-azabicyclo[2.2.1]heptan-2-yl)ethyl (6-methyl-5-(2-(1-methyl-1H-pyrazol-4-yl)pyrazolo[5,1-b]thiazole-7-carboxamido)pyridin-3-yl)carbamate (15.6 mg, yield 32%). LCMS (ESI): mass calcd. for C₂₅H₂₈N₈O₃S, 520.20; m/z found, 520.8 [M+H]⁺. ¹H NMR (METHANOL-d4) δ: 8.41-8.44 (m, 1H), 8.39 (s, 1H), 8.21 (s, 1H), 8.13 (d, J=2.4 Hz, 1H), 8.02 (s, 1H), 7.82 (s, 1H), 4.22-4.32 (m, 2H), 3.95 (s, 3H), 2.79-2.98 (m, 3H), 2.49 (s, 2H), 2.31-2.44 (m, 3H), 1.26-1.90 (m, 7H).

### Example 251. 2-(cyclohexyl(methyl)amino)ethyl (6-methyl-5-(2-(1-methyl-1H-pyrazol-4-yl)pyrazolo[5,1-b]thiazole-7-carboxamido)pyridin-3-yl)carbamate

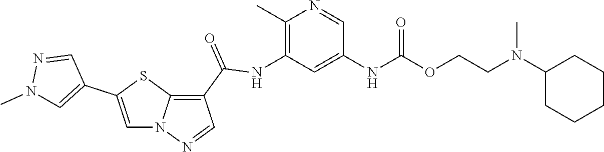 [SMILES: Cc1ncc(NC(=O)OCCN(C)C2CCCCC2)cc1NC(=O)c1cnn2cc(-c3cnn(C)c3)sc12]

To a mixture of N-(5-amino-2-methylpyridin-3-yl)-2-(1-methyl-1H-pyrazol-4-yl)pyrazolo[5,1-b]thiazole-7-carboxamide hydrochloride (40 mg, 0.0938 mmol) in DMF (0.7 mL) was added Et₃N (0.07 mL, 0.504 mmol) followed by CDI (35 mg, 0.216 mmol). The reaction was stirred at rt for 2 h. 2-(cyclohexyl(methyl)amino)ethan-1-ol (65 mg, 0.413 mmol) was then added and the reaction was heated at 80° C. by microwave for 1 h. To the reaction mixture was added aq. NaHCO₃ and the resulting mixture was extracted with 10% MeOH/CH₂Cl₂ (3×). The organic layer was dried over Na₂SO₄ and concentrated. The crude product was purified with silica gel column (14% MeOH/CH₂Cl₂ with 0.3-0.4% NH₄OH) to give 2-(cyclohexyl(methyl)amino)ethtyl(6-methyl-5-(2-(1-methyl-1H-pyrazol-4-yl)pyrazolo[5,1-b]thiazole-7-carboxamido)pyridin-3-yl)carbamate (16.2 mg, yield 32%). LCMS (ESI): mass calcd. for C₂₆H₃₂N₈O₃S, 536.23; m/z found, 536.8 [M+H]⁺. ¹H NMR (METHANOL-d4) δ: 8.39-8.44 (m, 1H), 8.39 (s, 1H), 8.21 (s, 1H), 8.13 (d, J=2.4 Hz, 1H), 8.02 (s, 1H), 7.82 (s, 1H), 4.26 (t, J=5.9 Hz, 2H), 3.95 (s, 3H), 2.76-2.88 (m, 2H), 2.43-2.54 (m, 4H), 2.37 (s, 3H), 1.75-1.95 (m, 4H), 1.54-1.75 (m, 1H), 1.10-1.37 (m, 5H).

### Example 252. 1-(tert-butyl)azetidin-3-yl (6-methyl-5-(2-(1-methyl-1H-pyrazol-4-yl)pyrazolo[5,1-b]thiazole-7-carboxamido)pyridin-3-yl)carbamate

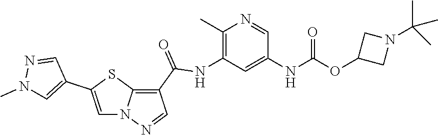 [SMILES: Cc1ncc(NC(=O)OC2CN(C(C)(C)C)C2)cc1NC(=O)c1cnn2cc(-c3cnn(C)c3)sc12]

To a mixture of N-(5-amino-2-methylpyridin-3-yl)-2-(1-methyl-1H-pyrazol-4-yl)pyrazolo[5,1-b]thiazole-7-carboxamide hydrochloride (40 mg, 0.0938 mmol) in DMF (0.7 mL) was added Et₃N (0.06 mL, 0.728 g/mL, 0.432 mmol) and CDI (30 mg, 0.185 mmol). The reaction was stirred at rt for 2 h. 1-(tert-butyl)azetidin-3-ol (55 mg, 0.426 mmol) was then added and the reaction was heated at 80° C. by microwave for 1 h. To the reaction mixture was added aq. NaHCO₃ and the resulting mixture was extracted with 10% MeOH/CH₂Cl₂ (3×). The organic layer was dried over Na₂SO₄ and concentrated. The crude product was purified with silica gel column (12% MeOH/CH₂Cl₂ with ˜0.3% NH₄OH) to give 1-(tert-butyl)azetidin-3-yl (6-methyl-5-(2-(1-methyl-1H-pyrazol-4-yl)pyrazolo[5,1-b]thiazole-7-carboxamido)pyridin-3-yl)carbamate (16.2 mg, yield 34%). LCMS (ESI): mass calcd. for C₂₄H₂₈N₈O₃S, 508.20; m/z found, 508.8 [M+H]⁺. ¹H NMR (METHANOL-d4) δ: 8.32-8.46 (m, 2H), 8.21 (s, 1H), 8.11 (d, J=2.4 Hz, 1H), 8.02 (s, 1H), 7.82 (s, 1H), 5.06 (t, J=5.9 Hz, 1H), 3.95 (s, 3H), 3.54-3.68 (m, 2H), 3.30-3.37 (m, 2H), 2.48 (s, 3H), 1.03 (s, 9H).

### Example 253. (S)-(1-methylpyrrolidin-2-yl)methyl (5-(2-(2-methoxypyridin-3-yl)pyrazolo[5,1-b]thiazole-7-carboxamido)-6-methylpyridin-3-yl)carbamate

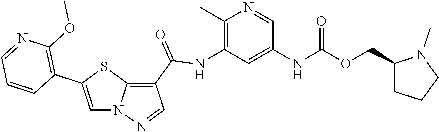 [SMILES: COc1ncccc1-c1cn2ncc(C(=O)Nc3cc(NC(=O)OC[C@@H]4CCCN4C)cnc3C)c2s1]

A mixture of N-(5-amino-2-methylpyridin-3-yl)-2-(2-methoxypyridin-3-yl)pyrazolo[5,1-b]thiazole-7-carboxamide (53 mg, 0.139 mmol), CDI (38 mg, 0.234 mmol), and Et₃N (0.07 mL, 0.504 mmol) in DMF (0.6 mL) was stirred for 2 h. (S)-(1-methylpyrrolidin-2-yl)methanol (60 mg, 0.521 mmol) was then added and the reaction was heated at 80° C. by microwave for 1 h. To the reaction mixture was added aq. NaHCO₃ and the resulting mixture was extracted with 10% MeOH/CH₂Cl₂ (3×). The organic layer was dried over Na₂SO₄ and concentrated. The crude product was purified with silica gel column (12% MeOH/CH₂Cl₂ with ˜0.3% NH₄OH) to give (S)-(1-methylpyrrolidin-2-yl)methyl (5-(2-(2-methoxypyridin-3-yl)pyrazolo[5,1-b]thiazole-7-carboxamido)-6-methylpyridin-3-yl)carbamate (30 mg, yield 41%). LCMS (ESI): mass calcd. for C₂₅H₂₇N₇O₄S, 521.18; m/z found, 521.8 [M+H]⁺. ¹H NMR (METHANOL-d4) δ: 8.52 (s, 1H), 8.34-8.44 (m, 2H), 8.08-8.20 (m, 2H), 7.95 (d, J=7.3 Hz, 1H), 7.03 (dd, J=7.8, 4.9 Hz, 1H), 4.11-4.24 (m, 2H), 4.09 (s, 3H), 3.07 (dt, J=9.5, 4.5 Hz, 1H), 2.52-2.67 (m, 1H), 2.48 (s, 3H), 2.46 (s, 3H), 2.27-2.37 (m, 1H), 2.03 (dq, J=12.5, 8.2 Hz, 1H), 1.73-1.86 (m, 2H), 1.58-1.70 (m, 1H).

### Example 254. 2-(2,2-dimethylpyrrolidin-1-yl)ethyl (5-(2-(2-methoxypyridin-3-yl)pyrazolo[5,1-b]thiazole-7-carboxamido)-6-methylpyridin-3-yl)carbamate

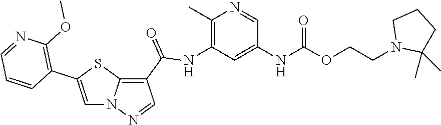 [SMILES: COc1ncccc1-c1cn2ncc(C(=O)Nc3cc(NC(=O)OCCN4CCCC4(C)C)cnc3C)c2s1]

A mixture of N-(5-amino-2-methylpyridin-3-yl)-2-(2-methoxypyridin-3-yl)pyrazolo[5,1-b]thiazole-7-carboxamide (60 mg, 0.158 mmol), CDI (40 mg, 0.247 mmol), and Et₃N (0.07 mL, 0.504 mmol) in DMF (0.5 mL) was stirred at rt for 2 h. A solution of 2-(2,2-dimethylpyrrolidin-1-yl)ethan-1-ol (70 mg, 0.489 mmol) in DMF (0.2 mL) was added and the reaction was heated at 80° C. by microwave for 45 min. To the reaction mixture was added aq. NaHCO₃ and the resulting mixture was extracted with 10% MeOH/CH₂Cl₂ (3×). The organic layer was dried over Na₂SO₄ and concentrated. The crude product was purified with silica gel column (12% MeOH/CH₂Cl₂ with ˜0.6% NH₄OH) to give 2-(2,2-dimethylpyrrolidin-1-yl)ethyl (5-(2-(2-methoxypyridin-3-yl)pyrazolo[5,1-b]thiazole-7-carboxamido)-6-methylpyridin-3-yl)carbamate (15.1 mg, yield 17%). LCMS (ESI): mass calcd. for C₂₇H₃₁N₇O₄S, 549.22; m/z found, 549.9 [M+H]⁺. ¹H NMR (METHANOL-d4) δ: 8.58 (s, 1H), 8.41 (s, 2H), 8.16 (td, J=4.6, 2.0 Hz, 2H), 8.00-8.04 (m, 1H), 7.07 (dd, J=7.3, 4.9 Hz, 1H), 4.26 (t, J=6.1 Hz, 2H), 4.12 (s, 3H), 2.82-2.98 (m, 2H), 2.74 (t, J=6.1 Hz, 2H), 2.49 (s, 3H), 1.77-1.86 (m, 2H), 1.64-1.72 (m, 2H), 1.04 (s, 6H).

### Example 255. 2-(pyrrolidin-1-yl)propyl (5-(2-(2-methoxypyridin-3-yl)pyrazolo[5,1-b]thiazole-7-carboxamido)-6-methylpyridin-3-yl)carbamate

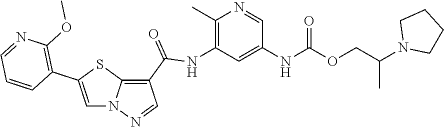 [SMILES: COc1ncccc1-c1cn2ncc(C(=O)Nc3cc(NC(=O)OCC(C)N4CCCC4)cnc3C)c2s1]

A mixture of N-(5-amino-2-methylpyridin-3-yl)-2-(2-methoxypyridin-3-yl)pyrazolo[5,1-b]thiazole-7-carboxamide (60 mg, 0.158 mmol), CDI (40 mg, 0.247 mmol), and Et₃N (0.07 mL, 0.504 mmol) in DMF (0.5 mL) was stirred at rt for 2 h. 2-(pyrrolidin-1-yl)propan-1-ol (70 mg, 0.542 mmol) was added and the reaction was heated at 80° C. by microwave for 45 min. To the reaction mixture was added aq. NaHCO₃ and the resulting mixture was extracted with 10% MeOH/CH₂Cl₂ (3×). The organic layer was dried over Na₂SO₄ and concentrated. The crude product was purified with silica gel column (12% MeOH/CH₂Cl₂ with ˜0.6% NH₄OH) to give 2-(pyrrolidin-1-yl)propyl (5-(2-(2-methoxypyridin-3-yl)pyrazolo[5,1-b]thiazole-7-carboxamido)-6-methylpyridin-3-yl)carbamate (19 mg, yield 22%). LCMS (ESI): mass calcd. for C₂₆H₂₉N₇O₄S, 535.20; m/z found, 535.8 [M+H]⁺. ¹H NMR (METHANOL-d4) δ: 8.57 (s, 1H), 8.38-8.45 (m, 2H), 8.12-8.20 (m, 2H), 8.00 (dd, J=7.3, 1.5 Hz, 1H), 7.06 (dd, J=7.3, 4.9 Hz, 1H), 4.25-4.34 (m, 1H), 4.08-4.18 (m, 4H), 2.62-2.77 (m, 5H), 2.49 (s, 3H), 1.73-1.91 (m, 4H), 1.21-1.27 (m, 3H).

### Example 256. (S)-(1-methylpyrrolidin-2-yl)methyl (5-(2-(2-hydroxypyridin-3-yl)pyrazolo[5,1-b]thiazole-7-carboxamido)-6-methylpyridin-3-yl)carbamate

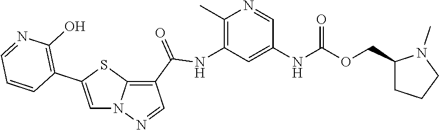 [SMILES: Cc1ncc(NC(=O)OC[C@@H]2CCCN2C)cc1NC(=O)c1cnn2cc(-c3cccnc3O)sc12]

### Step a: N-(5-amino-2-methylpyridin-3-yl)-2-(2-hydroxypyridin-3-yl)pyrazolo[5,1-b]thiazole-7-carboxamide

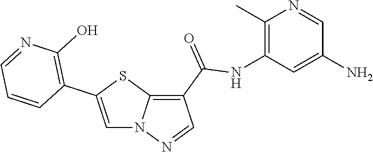 [SMILES: Cc1ncc(N)cc1NC(=O)c1cnn2cc(-c3cccnc3O)sc12]

A mixture of tert-butyl (5-(2-(2-methoxypyridin-3-yl)pyrazolo[5,1-b]thiazole-7-carboxamido)-6-methylpyridin-3-yl)carbamate (110 mg, 0.229 mmol) and HCl (4M in dioxane) (1 mL, 4 M, 4 mmol) in CH₂Cl₂ (3 mL) was stirred at rt for 1 h. The reaction mixture was diluted with diethyl ether and the resulting mixture was filtered. The solid was washed with ether and dried to give a mixture of N-(5-amino-2-methylpyridin-3-yl)-2-(2-hydroxypyridin-3-yl)pyrazolo[5,1-b]thiazole-7-carboxamide and N-(5-amino-2-methylpyridin-3-yl)-2-(2-methoxypyridin-3-yl)pyrazolo[5,1-b]thiazole-7-carboxamide, which will be used in next step without purification.

### Step b: (S)-(1-methylpyrrolidin-2-yl)methyl (5-(2-(2-hydroxypyridin-3-yl)pyrazolo[5,1-b]thiazole-7-carboxamido)-6-methylpyridin-3-yl)carbamate

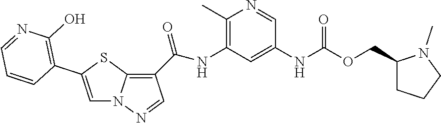 [SMILES: Cc1ncc(NC(=O)OC[C@@H]2CCCN2C)cc1NC(=O)c1cnn2cc(-c3cccnc3O)sc12]

To a mixture of N-(5-amino-2-methylpyridin-3-yl)-2-(2-hydroxypyridin-3-yl)pyrazolo[5,1-b]thiazole-7-carboxamide and N-(5-amino-2-methylpyridin-3-yl)-2-(2-methoxypyridin-3-yl)pyrazolo[5,1-b]thiazole-7-carboxamide (45 mg), CDI (30 mg, 0.185 mmol), and Et3N (0.07 mL, 0.504 mmol) in DMF (0.5 mL) was stirred at rt for 2 h. (S)-(1-methylpyrrolidin-2-yl)methanol (45 mg, 0.391 mmol) was then added and the reaction was heated at 80° C. by microwave for 1 h. To the reaction mixture was added aq. NaHCO₃ and the resulting mixture was extracted with 10% MeOH/CH₂Cl₂ (3×). The organic layer was dried over Na₂SO₄ and concentrated. The crude product was purified with silica gel column (16% MeOH/CH₂Cl₂ with ˜0.4% NH₄OH) to give (S)-(1-methylpyrrolidin-2-yl)methyl (5-(2-(2-hydroxypyridin-3-yl)pyrazolo[5,1-b]thiazole-7-carboxamido)-6-methylpyridin-3-yl)carbamate (6.2 mg, yield 12%). LCMS (ESI): mass calcd. for C₂₄H₂₅N₇O₄S, 507.17; m/z found, 507.8 [M+H]⁺. ¹H NMR (METHANOL-d4) δ: 8.81 (s, 1H), 8.36-8.48 (m, 2H), 8.00-8.20 (m, 1H), 7.97 (dd, J=7.1, 1.7 Hz, 1H), 7.45 (dd, J=6.4, 2.0 Hz, 1H), 6.52 (t, J=6.8 Hz, 1H), 4.20 (d, J=4.9 Hz, 2H), 3.11 (dt, J=9.7, 4.7 Hz, 1H), 2.62-2.72 (m, 1H), 2.50 (m, 6H), 2.29-2.46 (m, 1H), 1.95-2.15 (m, 1H), 1.77-1.87 (m, 2H), 1.64-1.77 (m, 1H).

### Example 257. (S)-(1-methylpyrrolidin-2-yl)methyl (6-methyl-5-(2-(1-methyl-1H-indazol-4-yl)pyrazolo[5,1-b]thiazole-7-carboxamido)pyridin-3-yl)carbamate

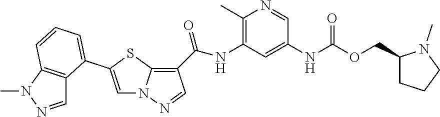 [SMILES: Cc1ncc(NC(=O)OC[C@@H]2CCCN2C)cc1NC(=O)c1cnn2cc(-c3cccc4c3cnn4C)sc12]

### Step a: (S)-(1-methylpyrrolidin-2-yl)methyl (5-(2-bromopyrazolo[5,1-b]thiazole-7-carboxamido)-6-methylpyridin-3-yl)carbamate

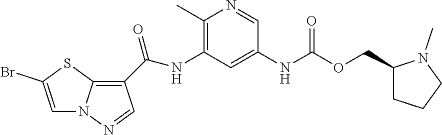 [SMILES: Cc1ncc(NC(=O)OC[C@@H]2CCCN2C)cc1NC(=O)c1cnn2cc(Br)sc12]

To a mixture of N-(5-amino-2-methylpyridin-3-yl)-2-bromopyrazolo[5,1-b]thiazole-7-carboxamide hydrochloride (200 mg, 0.515 mmol) in DMF (0.7 mL) was added Et₃N (0.32 mL, 2.30 mmol) and CDI (125 mg, 0.771 mmol). The reaction was stirred at rt for 2 h. (S)-(1-methylpyrrolidin-2-yl)methanol (220 mg, 1.91 mmol) was then added and the reaction was heated at 80° C. by microwave for 1 h. To the reaction mixture was added aq. NaHCO₃ and the resulting mixture was extracted with 10% MeOH/CH₂Cl₂ (3×). The organic layer was dried over Na₂SO₄ and concentrated. The crude product was purified with silica gel column (14% MeOH/CH₂Cl₂ with 0.3˜0.4% NH₄OH) to give (S)-(1-methylpyrrolidin-2-yl)methyl (5-(2-bromopyrazolo[5,1-b]thiazole-7-carboxamido)-6-methylpyridin-3-yl)carbamate (160 mg, yield 63.022%). LCMS (ESI): mass calcd. for C₁₉H₂₁BrN₆O₃S, 492.06; m/z found, 492.8 [M+H]⁺.

### Step b: (S)-(1-methylpyrrolidin-2-yl)methyl (6-methyl-5-(2-(1-methyl-1H-indazol-4-yl)pyrazolo[5,1-b]thiazole-7-carboxamido)pyridin-3-yl)carbamate

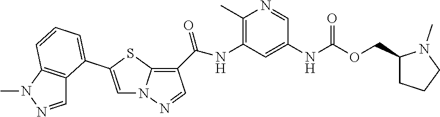 [SMILES: Cc1ncc(NC(=O)OC[C@@H]2CCCN2C)cc1NC(=O)c1cnn2cc(-c3cccc4c3cnn4C)sc12]

A mixture of (S)-(1-methylpyrrolidin-2-yl)methyl (5-(2-bromopyrazolo[5,1-b]thiazole-7-carboxamido)-6-methylpyridin-3-yl)carbamate (30 mg, 0.061 mmol), 1-methyl-4-(4,4,5,5-tetramethyl-1,3,2-dioxaborolan-2-yl)-1H-indazole (22 mg, 0.085 mmol), Cs₂CO₃ (40 mg, 0.123 mmol), and PdCl₂(dppf) (5 mg, 0.0068 mmol) in 1,4-dioxane (0.75 mL) and H₂O (0.15 mL) was heated at 120° C. by microwave under argon for 1 h. Additional PdCl₂(dppf) (4 mg, 0.00546 mmol) was added and the reaction was heated at 120° C. for another 1.5 h. The reaction was diluted with CH₂Cl₂ and washed with H₂O. The organic layer was dried over Na₂SO₄ and concentrated. The residue was purified with silica gel column (10% MeOH/CH₂Cl₂ with ˜0.25% NH₄OH) to give (S)-(1-methylpyrrolidin-2-yl)methyl (6-methyl-5-(2-(1-methyl-1H-indazol-4-yl)pyrazolo[5,1-b]thiazole-7-carboxamido)pyridin-3-yl)carbamate (8.8 mg, yield 27%). LCMS (ESI): mass calcd. for C₂₇H₂₈N₈O₃S, 544.20; m/z found, 544.8 [M+H]⁺. ¹H NMR (METHANOL-d4) δ: 8.53 (s, 1H), 8.47 (s, 1H), 8.41-8.45 (m, 1H), 8.35-8.36 (m, 1H), 8.14 (br d, J=2.4 Hz, 1H), 7.61 (d, J=8.8 Hz, 1H), 7.46-7.55 (m, 1H), 7.37-7.46 (m, 1H), 4.19 (d, J=4.9 Hz, 2H), 4.11-4.16 (m, 3H), 3.04-3.13 (m, 1H), 2.61 (br d, J=7.8 Hz, 1H), 2.51 (s, 3H), 2.47 (s, 3H), 2.34 (q, J=9.0 Hz, 1H), 1.93-2.16 (m, 1H), 1.76-1.85 (m, 2H), 1.63-1.76 (m, 1H).

### Example 258. (S)-(1-methylpyrrolidin-2-yl)methyl (5-(2-(1H-pyrrol-3-yl)pyrazolo[5,1-b]thiazole-7-carboxamido)-6-methylpyridin-3-yl)carbamate

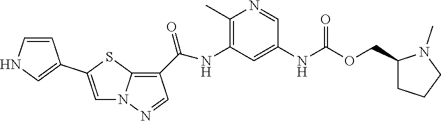 [SMILES: Cc1ncc(NC(=O)OC[C@@H]2CCCN2C)cc1NC(=O)c1cnn2cc(-c3cc[nH]c3)sc12]

A mixture of (S)-(1-methylpyrrolidin-2-yl)methyl (5-(2-bromopyrazolo[5,1-b]thiazole-7-carboxamido)-6-methylpyridin-3-yl)carbamate (52 mg, 0.105 mmol), 3-(4,4,5,5-tetramethyl-1,3,2-dioxaborolan-2-yl)-1H-pyrrole (30 mg, 0.155 mmol), PdCl₂(dppf) (8 mg, 0.0109 mmol), and Cs₂CO₃ (70 mg, 0.215 mmol) in 1,4-dioxane (1 mL) and H₂O (0.2 mL) was heated by microwave at 130° C. for 30 min. Additional PdCl₂(dppf) (3 mg, 0.00416 mmol) was added and the reaction was heated by microwave at 120° C. for another 30 min. The reaction mixture was diluted with 10% MeOH/CH₂Cl₂ and washed with aq. NaHCO₃. The aqueous layer was extracted with 10% MeOH/CH₂Cl₂ (2×). The organic layers were combined, dried over Na₂SO₄, and concentrated. The crude product was purified with silica gel column (12% MeoH/CH₂Cl₂ with 0.30% NH₄OH) to give (S)-(1-methylpyrrolidin-2-yl)methyl (5-(2-(1H-pyrrol-3-yl)pyrazolo[5,1-b]thiazole-7-carboxamido)-6-methylpyridin-3-yl)carbamate (9.2 mg, yield 18%). LCMS (ESI): mass calcd. for C₂₃H₂₅N₇O₃S, 479.17; m/z found, 479.9 [M+H]⁺. ¹H NMR (METHANOL-d4) δ: 8.40-8.50 (m, 1H), 8.36 (s, 1H), 8.08-8.16 (m, 1H), 8.03 (s, 1H), 7.16 (t, J=1.7 Hz, 1H), 6.72-6.98 (m, 1H), 6.43 (dd, J=2.7, 1.7 Hz, 1H), 4.15-4.30 (m, 2H), 3.06-3.23 (m, 1H), 2.69-2.79 (m, 1H), 2.53 (s, 3H), 2.49 (s, 3H), 2.37-2.46 (m, 1H), 2.01-2.16 (m, 1H), 1.79-1.90 (m, 2H), 1.65-1.77 (m, 1H).

### Example 259. N-(5-(2-(2,2-dimethylpyrrolidin-1-yl)acetamido)-2-methylpyridin-3-yl)-2-(1H-pyrazolo[3,4-b]pyridin-1-yl)pyrazolo[5,1-b]thiazole-7-carboxamide

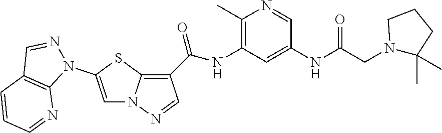 [SMILES: Cc1ncc(NC(=O)CN2CCCC2(C)C)cc1NC(=O)c1cnn2cc(-n3ncc4cccnc43)sc12]

### Step a: ethyl 2-(1H-pyrazolo[3,4-b]pyridin-1-yl)pyrazolo[5,1-b]thiazole-7-carboxylate

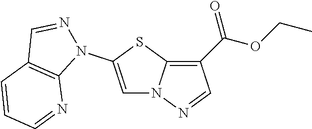 [SMILES: CCOC(=O)c1cnn2cc(-n3ncc4cccnc43)sc12]

A mixture of ethyl 2-bromopyrazolo[5,1-b]thiazole-7-carboxylate (200 mg, 0.727 mmol), 1H-pyrazolo[3,4-b]pyridine (110 mg, 0.923 mmol), CuI (40 mg, 0.21 mmol), trans-N,N′-dimethylcyclohexane-1,2-diamine (60 mg, 0.422 mmol), and Cs₂CO₃ (350 mg, 1.074 mmol) in 1,4-dioxane (5 mL) was heated at 120° C. by microwave under argon for 10 h. The reaction mixture was diluted with CH₂Cl₂ and filtered. The solution was concentrated and the residue was purified with silica gel column (30% EtOAc/heptane) to give ethyl 2-(1H-pyrazolo[3,4-b]pyridin-1-yl)pyrazolo[5,1-b]thiazole-7-carboxylate (147 mg, yield 65%). LCMS (ESI): mass calcd. for C₁₄H₁₁N₅O₂S, 313.06; m/z found, 313.8 [M+H]⁺.

### Step b: 2-(1H-pyrazolo[3,4-b]pyridin-1-yl)pyrazolo[5,1-b]thiazole-7-carboxylic acid

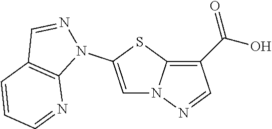 [SMILES: O=C(O)c1cnn2cc(-n3ncc4cccnc43)sc12]

A mixture of ethyl 2-(1H-pyrazolo[3,4-b]pyridin-1-yl)pyrazolo[5,1-b]thiazole-7-carboxylate (84 mg, 0.268 mmol) and LiOH (20 mg, 0.835 mmol) in THE (3 mL) and H₂O (1.5 mL) was stirred at 65° C. for 6 h. To the reaction was added 2N HCl to adjust pH to 3-4. The resulting mixture was concentrated to remove most of organic solvent and was then filtered. The solid was washed with H₂O and dried to give 2-(1H-pyrazolo[3,4-b]pyridin-1-yl)pyrazolo[5,1-b]thiazole-7-carboxylic acid (69 mg, yield 90%).

### Step c: N-(5-(2-(2,2-dimethylpyrrolidin-1-yl)acetamido)-2-methylpyridin-3-yl)-2-(1H-pyrazolo[3,4-b]pyridin-1-yl)pyrazolo[5,1-b]thiazole-7-carboxamide

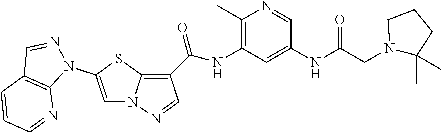 [SMILES: Cc1ncc(NC(=O)CN2CCCC2(C)C)cc1NC(=O)c1cnn2cc(-n3ncc4cccnc43)sc12]

A mixture of 2-(1H-pyrazolo[3,4-b]pyridin-1-yl)pyrazolo[5,1-b]thiazole-7-carboxylic acid (30 mg, 0.105 mmol), N-(5-amino-6-methylpyridin-3-yl)-2-(2,2-dimethylpyrrolidin-1-yl)acetamide (32 mg, 0.122 mmol), and EDCI (30 mg, 0.156 mmol) in pyridine (1.5 mL) was heated at 70° C. for 5 h. The reaction mixture was concentrated. The residue was diluted with 10% MeOH/CH₂Cl₂ and washed with aq. NaHCO₃. The aqueous layer was extracted again with 10% MeOH/CH₂Cl₂. The organic layers were combined, dried over Na₂SO₄, and concentrated. The crude product was purified with silica gel column (5% MeOH/CH₂Cl₂ with ˜0.25% NH₄OH) to give N-(5-(2-(2,2-dimethylpyrrolidin-1-yl)acetamido)-2-methylpyridin-3-yl)-2-(1H-pyrazolo[3,4-b]pyridin-1-yl)pyrazolo[5,1-b]thiazole-7-carboxamide (17.9 mg, yield 32.14%). LCMS (ESI): mass calcd. for C₂₆H₂₇N₉O₂S, 529.20; m/z found, 530.2 [M+H]⁺. ¹H NMR (CHLOROFORM-d) δ: 9.29 (br s, 1H), 8.83 (s, 1H), 8.68 (dd, J=4.4, 1.5 Hz, 1H), 8.60-8.65 (m, 1H), 8.40 (d, J=2.0 Hz, 1H), 8.23 (s, 1H), 8.18 (s, 1H), 8.11-8.17 (m, 1H), 7.83 (s, 1H), 7.30 (dd, J=8.1, 4.6 Hz, 1H), 3.21 (s, 2H), 2.87 (t, J=7.1 Hz, 2H), 2.54 (s, 3H), 1.71-1.92 (m, 4H), 1.08 (s, 6H).

### Example 260. N-(5-(3-(2,2-dimethylpyrrolidin-1-yl)propanamido)-2-methylpyridin-3-yl)-2-(1H-pyrazolo[3,4-b]pyridin-1-yl)pyrazolo[5,1-b]thiazole-7-carboxamide

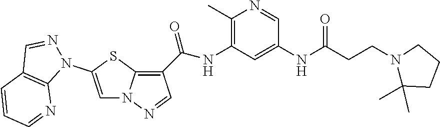 [SMILES: Cc1ncc(NC(=O)CCN2CCCC2(C)C)cc1NC(=O)c1cnn2cc(-n3ncc4cccnc43)sc12]

### Step a: N-(5-acrylamido-2-methylpyridin-3-yl)-2-bromopyrazolo[5,1-b]thiazole-7-carboxamide

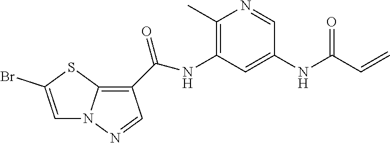 [SMILES: C=CC(=O)Nc1cnc(C)c(NC(=O)c2cnn3cc(Br)sc23)c1]

To a mixture of N-(5-amino-2-methylpyridin-3-yl)-2-bromopyrazolo[5,1-b]thiazole-7-carboxamide hydrochloride (200 mg, 0.515 mmol) and Et₃N (0.22 mL, 1.58 mmol) in CH₂Cl₂ (6 mL) at 0° C. was added 3-chloropropanoyl chloride (0.08 mL, 0.838 mmol). The reaction was warmed up to rt and stirred overnight. The reaction mixture was concentrated and the residue was suspended in H₂O and filtered. The solid was dried to give crude N-(5-acrylamido-2-methylpyridin-3-yl)-2-bromopyrazolo[5,1-b]thiazole-7-carboxamide (200 mg, yield 96%), which will be used in next step without purification.

### Step b: 2-bromo-N-(5-(3-(2,2-dimethylpyrrolidin-1-yl)propanamido)-2-methylpyridin-3-yl)pyrazolo[5,1-b]thiazole-7-carboxamide

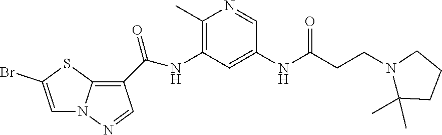 [SMILES: Cc1ncc(NC(=O)CCN2CCCC2(C)C)cc1NC(=O)c1cnn2cc(Br)sc12]

A solution of N-(5-acrylamido-2-methylpyridin-3-yl)-2-bromopyrazolo[5,1-b]thiazole-7-carboxamide (120 mg, 0.295 mmol), 2,2-dimethylpyrrolidine (60 mg, 0.605 mmol), and Et₃N (0.1 mL, 0.719 mmol) in ¹PrOH (3 mL) was heated at 100° C. by microwave for 6 h. The reaction mixture was concentrated. The residue was purified with silica gel column (10% MeOH/CH₂Cl₂ with ˜0.5% NH₄OH) to give 2-bromo-N-(5-(3-(2,2-dimethylpyrrolidin-1-yl)propanamido)-2-methylpyridin-3-yl)pyrazolo[5,1-b]thiazole-7-carboxamide (125 mg, yield 84%). LCMS (ESI): mass calcd. for C₂₁H₂₅BrN₆O₂S, 504.09; m/z found, 504.8 [M+H]⁺.

### Step c: N-(5-(3-(2,2-dimethylpyrrolidin-1-yl)propanamido)-2-methylpyridin-3-yl)-2-(1H-pyrazolo[3,4-b]pyridin-1-yl)pyrazolo[5,1-b]thiazole-7-carboxamide

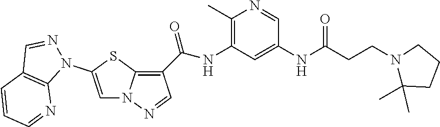 [SMILES: Cc1ncc(NC(=O)CCN2CCCC2(C)C)cc1NC(=O)c1cnn2cc(-n3ncc4cccnc43)sc12]

A mixture of 2-bromo-N-(5-(3-(2,2-dimethylpyrrolidin-1-yl)propanamido)-2-methylpyridin-3-yl)pyrazolo[5,1-b]thiazole-7-carboxamide (50 mg, 0.0989 mmol), 1H-pyrazolo[3,4-b]pyridine (17 mg, 0.143 mmol), CuI (10 mg, 0.0525 mmol), trans-N,N′-dimethylcyclohexane-1,2-diamine (15 mg, 0.105 mmol), and Cs₂CO₃ (65 mg, 0.199 mmol) in 1,4-dioxane (1.5 mL) was heated at 120° C. by microwave for 10 h. The reaction mixture was diluted with CH₂Cl₂ and filtered. The solution was concentrated. The residue was purified with silica gel column (12% MeOH/CH₂Cl₂ with ˜0.6% NH₄OH). The product was further purified by HPLC (30-70% CH₃CN/H₂O with 10 mM NH₄OH, 6 min) to give N-(5-(3-(2,2-dimethylpyrrolidin-1-yl)propanamido)-2-methylpyridin-3-yl)-2-(1H-pyrazolo[3,4-b]pyridin-1-yl)pyrazolo[5,1-b]thiazole-7-carboxamide (15.3 mg, yield 28%). LCMS (ESI): mass calcd. for C₂₇H₂₉N₉O₂S, 543.22; m/z found, 543.9 [M+H]⁺. ¹H NMR (METHANOL-d4) δ: 8.78 (s, 1H), 8.64-8.73 (m, 1H), 8.53 (d, J=2.4 Hz, 1H), 8.38 (s, 1H), 8.24-8.34 (m, 3H), 7.36 (dd, J=8.1, 4.6 Hz, 1H), 2.86 (dt, J=16.6, 7.3 Hz, 4H), 2.59 (t, J=7.1 Hz, 2H), 2.51 (s, 3H), 1.79-1.91 (m, 2H), 1.70-1.79 (m, 2H), 1.09 (s, 6H).

### Example 261. N-(5-(2-(3,3-dimethylazetidin-1-yl)acetamido)-2-methylpyridin-3-yl)-2-(1-(2-methoxyethyl)-1H-pyrazol-4-yl)pyrazolo[5,1-b]thiazole-7-carboxamide

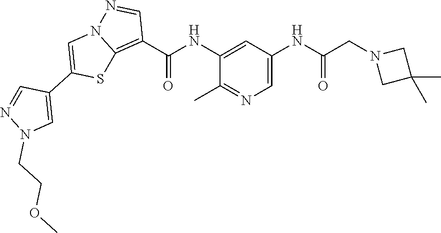 [SMILES: COCCn1cc(-c2cn3ncc(C(=O)Nc4cc(NC(=O)CN5CC(C)(C)C5)cnc4C)c3s2)cn1]

### Step a: N-(5-amino-2-methylpyridin-3-yl)-2-bromopyrazolo[5,1-b]thiazole-7-carboxamide

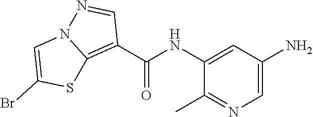 [SMILES: Cc1ncc(N)cc1NC(=O)c1cnn2cc(Br)sc12]

A solution of tert-butyl (5-(2-bromopyrazolo[5,1-b]thiazole-7-carboxamido)-6-methylpyridin-3-yl)carbamate (777 mg, 1.72 mmol) in 25% TFA/CH₂Cl₂ (25 mL) was stirred at 25° C. for 3 h. All solvents were removed in vacuo. The residue was taken up in toluene (25 mL) and all solvents were removed in vacuo (2×). The residue was dried under high vacuum to afford the TFA salt of the product, N-(5-amino-2-methylpyridin-3-yl)-2-bromopyrazolo[5,1-b]thiazole-7-carboxamide, as a tan solid (quantitative yield). The product was used without further purification. LCMS (ESI): mass calcd. for C₁₂H₁₀BrN₅OS, 351.0/353.0; m/z found, 352.0/354.0 [M+H]⁺. ¹H NMR (400 MHz, DMSO-d6) δ ppm 2.45 (s, 3H), 6.26 (br s, 2H), 7.80 (d, J=1.00 Hz, 1H), 7.83 (d, J=1.00 Hz, 1H), 8.64 (s, 1H), 8.82 (s, 1H), 10.18-10.33 (m, 1H).

### Step b: 2-bromo-N-(5-(2-chloroacetamido)-2-methylpyridin-3-yl)pyrazolo[5,1-b]thiazole-7-carboxamide

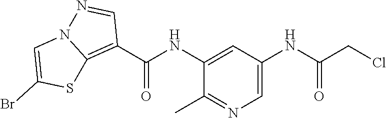 [SMILES: Cc1ncc(NC(=O)CCl)cc1NC(=O)c1cnn2cc(Br)sc12]

To a suspension of N-(5-amino-2-methylpyridin-3-yl)-2-bromopyrazolo[5,1-b]thiazole-7-carboxamide (605.10 mg, 1.72 mmol) in DCM (10 mL) was added Et₃N (0.72 mL, 0.73 g/mL, 5.15 mmol) followed by the addition of chloroacetyl chloride (0.15 mL, 1.42 g/mL, 1.89 mmol). The reaction was stirred at 25° C. under argon for 17 h. Another 0.5 equivalent of chloroacetyl chloride (0.076 mL, 1.42 g/mL, 0.95 mmol) was added. The reaction was maintained stirring at 25° C. under argon for 2 h. All solvents were removed in vacuo. The residue was taken up in toluene (25 mL) and all solvents were removed in vacuo. The residue was taken up in 20% MeOH/CH₂Cl₂ (25 mL) and silica gel (6 g). All solvents were removed in vacuo. The silica gel mesh was loaded on a SiliCycle silica gel cartridge (40 g) eluting with MeOH/CH₂Cl₂ (0-30%) over 30 min to afford a mixture of products. The mixture was taken up in 20% MeOH/CH₂Cl₂ (25 mL) and silica gel (6 g) and concentrated in vacuo. The silica gel mesh was loaded on a SiliCycle silica gel cartridge (40 g) eluting with 100% EtOAc over 30 min to afford the product, 2-bromo-N-(5-(2-chloroacetamido)-2-methylpyridin-3-yl)pyrazolo[5,1-b]thiazole-7-carboxamide, as a tan solid (402.9 mg). LCMS (ESI): mass calcd. for C₁₄H₁₁BrClN₅O₂S, 427.0/429.0; m/z found, 428.0/429.9 [M+H]⁺. ¹H NMR (400 MHz, DMSO-d6) δ ppm 2.40 (s, 3H), 4.29 (s, 2H), 8.14 (d, J=1.00 Hz, 1H), 8.52 (d, J=1.00 Hz, 1H), 8.62 (s, 1H), 8.79 (s, 1H), 9.93 (s, 1H), 10.53 (s, 1H).

### Step c: 2-bromo-N-(5-(2-(3,3-dimethylazetidin-1-yl)acetamido)-2-methylpyridin-3-yl)pyrazolo[5,1-b]thiazole-7-carboxamide

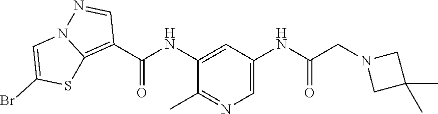 [SMILES: Cc1ncc(NC(=O)CN2CC(C)(C)C2)cc1NC(=O)c1cnn2cc(Br)sc12]

A mixture of 2-bromo-N-(5-(2-chloroacetamido)-2-methylpyridin-3-yl)pyrazolo[5,1-b]thiazole-7-carboxamide (2 g, 4.67 mmol), 3,3-dimethylazetidine hydrochloride (0.68 g, 5.60 mmol), K₂CO₃ (2.58 g, 18.66 mmol), and NaI (250 mg) in DMF (25 mL) was heated at 50° C. for 23 h. The reaction was poured into water (450 mL) with stirring. The reaction was filtered and the collected precipitate was air dried then dried under high vacuum to afford the product, 2-bromo-N-(5-(2-(3,3-dimethylazetidin-1-yl)acetamido)-2-methylpyridin-3-yl)pyrazolo[5,1-b]thiazole-7-carboxamide, as a tan solid (2.181 g). The product was used without further purification. LCMS (ESI): mass calcd. for C₁₉H₂₁BrN₆O₂S, 476.1/478.1; m/z found, 477.1/479.1 [M+H]⁺. ¹H NMR (400 MHz, DMSO-d6) δ ppm 1.20 (s, 6H), 2.38 (s, 3H), 3.04 (s, 4H), 3.20 (s, 2H), 8.13 (d, J=1.00 Hz, 1H), 8.55 (d, J=1.00 Hz, 1H), 8.60 (s, 1H), 8.78 (s, 1H), 9.81 (s, 1H), 9.95 (s, 1H).

### Step d: N-(5-(2-(3,3-dimethylazetidin-1-yl)acetamido)-2-methylpyridin-3-yl)-2-(1-(2-methoxyethyl)-1H-pyrazol-4-yl)pyrazolo[5,1-b]thiazole-7-carboxamide

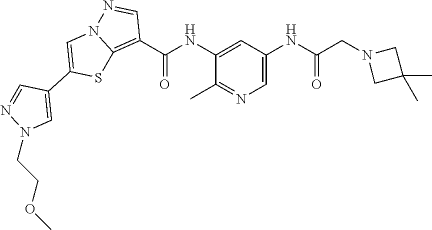 [SMILES: COCCn1cc(-c2cn3ncc(C(=O)Nc4cc(NC(=O)CN5CC(C)(C)C5)cnc4C)c3s2)cn1]

A mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 1-(2-methoxyethyl)-4-(4,4,5,5-tetramethyl-1,3,2-dioxaborolan-2-yl)-1H-pyrazole (79.22 mg, 0.31 mmol), DPPF PD G4 (19.65 mg, 0.021 mmol), and Cs₂CO₃ (204.75 mg, 0.63 mmol) in 1,4-dioxane (2.5 mL) and water (0.5 mL) was thoroughly flushed with argon before being capped and heated at 90° C. 19.5 h. LCMS indicated a mixture of the starting bromide and product (1:1). The reaction was diluted with MeOH (25 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min and 30% MeOH/CH₂Cl₂ over 5 min to afford the product, N-(5-(2-(3,3-dimethylazetidin-1-yl)acetamido)-2-methylpyridin-3-yl)-2-(1-(2-methoxyethyl)-1H-pyrazol-4-yl)pyrazolo[5,1-b]thiazole-7-carboxamide, as a dark brown solid (25 mg). LCMS (ESI): mass calcd. for C₂₅H₃₀N₈O₃S, 522.2; m/z found, 523.2 [M+H]⁺; ¹H NMR (400 MHz, DMSO-d6) δ ppm 1.21 (s, 6H), 2.39 (s, 3H), 3.12 (br s, 4H), 3.24 (s, 3H), 3.33 (br s, 2H), 3.70 (t, J=1.00 Hz, 2H), 4.29 (t, J=1.00 Hz, 2H), 7.90 (s, 1H), 8.12 (d, J=1.00 Hz, 1H), 8.22 (s, 1H), 8.53 (s, 1H), 8.56 (d, J=1.00 Hz, 1H), 8.60 (s, 1H), 9.89 (s, 1H), 9.98 (br s, 1H).

### Example 262: N-(5-(2-(3,3-dimethylazetidin-1-yl)acetamido)-2-methylpyridin-3-yl)-2-(1-(1,1-dioxidothietan-3-yl)-1H-pyrazol-4-yl)pyrazolo[5,1-b]thiazole-7-carboxamide

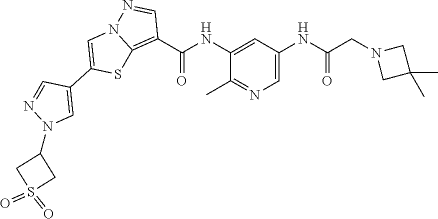 [SMILES: Cc1ncc(NC(=O)CN2CC(C)(C)C2)cc1NC(=O)c1cnn2cc(-c3cnn(C4CS(=O)(=O)C4)c3)sc12]

A mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 3-(4-(4,4,5,5-tetramethyl-1,3,2-dioxaborolan-2-yl)-1H-pyrazol-1-yl)thietane 1,1-dioxide (93.69 mg, 0.31 mmol), DPPF PD G4 (19.65 mg, 0.021 mmol), and Cs₂CO₃ (204.75 mg, 0.63 mmol) in 1,4-dioxane (2.5 mL) and water (0.5 mL) was thoroughly flushed with argon before being capped and heated at 90° C. for 17 h. LCMS indicated a mixture of the starting bromide and product (1:1). The reaction was diluted with MeOH (25 mL) and silica gel added (3 g). All solvents were removed in vacuo. The silica gel mesh was loaded on a Redi Sep Rf silica gel cartridge (24 g) eluting with MeOH/CH₂Cl₂ (15-30%) over 15 min to afford the product, N-(5-(2-(3,3-dimethylazetidin-1-yl)acetamido)-2-methylpyridin-3-yl)-2-(1-(1,1-dioxidothietan-3-yl)-1H-pyrazol-4-yl)pyrazolo[5,1-b]thiazole-7-carboxamide, as a brown solid (12.5 mg). LCMS (ESI): mass calcd. for C₂₅H₂₈N₈O₄S₂, 568.2; m/z found, 569.3 [M+H]⁺; ¹H NMR (400 MHz, DMSO-d6) δ ppm 1.20 (s, 6H), 2.38 (s, 3H), 3.04 (s, 4H), 3.22 (s, 2H), 4.65-4.76 (m, 2H), 4.77-4.88 (m, 2H), 5.33-5.47 (m, 1H), 8.08 (s, 1H), 8.14 (d, J=1.00 Hz, 1H), 8.42 (s, 1H), 8.51 (s, 1H), 8.57 (d, J=1.00 Hz, 1H), 8.66 (s, 1H), 9.82 (s, 1H), 9.90 (s, 1H).

### Example 263: N-(5-(2-(3,3-dimethylazetidin-1-yl)acetamido)-2-methylpyridin-3-yl)-2-(1-(oxetan-3-ylmethyl)-1H-pyrazol-4-yl)pyrazolo[5,1-b]thiazole-7-carboxamide

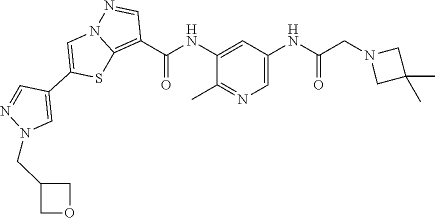 [SMILES: Cc1ncc(NC(=O)CN2CC(C)(C)C2)cc1NC(=O)c1cnn2cc(-c3cnn(CC4COC4)c3)sc12]

A mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 1-(oxetan-3-ylmethyl)-4-(4,4,5,5-tetramethyl-1,3,2-dioxaborolan-2-yl)-1H-pyrazole (66.40 mg, 0.25 mmol), DPPF PD G4 (19.65 mg, 0.021 mmol), and Cs₂CO₃ (204.75 mg, 0.63 mmol) in 1,4-dioxane (2.5 mL) and water (0.5 mL) was thoroughly flushed with argon before being capped and heated at 90° C. for 17 h, LCMS indicated a mixture of the starting bromide and product (2:1). The reaction was diluted with MeOH (25 mL) and silica gel added (3 g). All solvents were removed in vacuo. The silica gel mesh was loaded on a Redi Sep Rf silica gel cartridge (24 g) eluting with MeOH/CH₂Cl₂ (15-30%) over 15 min to afford the product, N-(5-(2-(3,3-dimethylazetidin-1-yl)acetamido)-2-methylpyridin-3-yl)-2-(1-(oxetan-3-ylmethyl)-1H-pyrazol-4-yl)pyrazolo[5,1-b]thiazole-7-carboxamide, as a dark brown solid (20.4 mg). LCMS (ESI): mass calcd. for C₂₆H₃₀N₈O₃S, 534.2; m/z found, 535.3 [M+H]⁺; ¹H NMR (400 MHz, DMSO-d6) δ ppm 1.20 (s, 6H), 2.39 (s, 3H), 3.04 (s, 4H), 3.22 (s, 2H), 3.37-3.52 (m, 1H), 4.37-4.49 (m, 4H), 4.65 (t, J=1.00 Hz, 2H), 7.90 (s, 1H), 8.13 (d, J=1.00 Hz, 1H), 8.26 (s, 1H), 8.50 (s, 1H), 8.55 (d, J=1.00 Hz, 1H), 8.59 (s, 1H), 9.82 (s, 1H), 9.90 (s, 1H).

### Example 264: N-(5-(2-(3,3-dimethylazetidin-1-yl)acetamido)-2-methylpyridin-3-yl)-2-(1-(oxetan-3-yl)-1H-pyrazol-4-yl)pyrazolo[5,1-b]thiazole-7-carboxamide

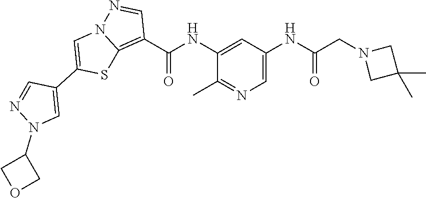 [SMILES: Cc1ncc(NC(=O)CN2CC(C)(C)C2)cc1NC(=O)c1cnn2cc(-c3cnn(C4COC4)c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 1-(oxetan-3-yl)-4-(4,4,5,5-tetramethyl-1,3,2-dioxaborolan-2-yl)-1H-pyrazole (62.87 mg, 0.25 mmol), and DPPF PD G4 (19.65 mg, 0.021 mmol) in 1,4-dioxane (2.5 mLl) and water (0.5 mL) was added DPPF PD G4 (19.65 mg, 0.021 mmol). The reaction mixture was thoroughly flushed with argon before being capped and heated at 90° C. For 23 h. The reaction was diluted with MeOH (25 mL) and silica gel added (3 g). All solvents were removed in vacuo. The silica gel mesh was loaded on a Redi Sep Rf silica gel cartridge (24 g) eluting with MeOH/CH₂Cl₂ (15-30%) over 15 min to afford the product, N-(5-(2-(3,3-dimethylazetidin-1-yl)acetamido)-2-methylpyridin-3-yl)-2-(1-(oxetan-3-yl)-1H-pyrazol-4-yl)pyrazolo[5,1-b]thiazole-7-carboxamide, as a brown solid (24.3 mg). LCMS (ESI): mass calcd. for C₂₅H₂₈N₈O₃S, 520.2; m/z found, 521.3 [M+H]⁺; ¹H NMR (400 MHz, DMSO-d6) δ ppm 1.21 (s, 6H), 2.39 (s, 3H), 3.08-3.24 (m, 4H), 3.40 (br s, 2H), 4.84-4.99 (m, 4H), 5.61 (quin, J=1.00 Hz, 1H), 8.05 (s, 1H), 8.12 (d, J=1.00 Hz, 1H), 8.42 (s, 1H), 8.52 (s, 1H), 8.57 (d, J=1.00 Hz, 1H), 8.63 (s, 1H), 9.89 (s, 1H), 10.00 (br s, 1H).

### Example 165: N-(5-(2-(3,3-dimethylazetidin-1-yl)acetamido)-2-methylpyridin-3-yl)-2-(1-((methylsulfonyl)methyl)-1H-pyrazol-4-yl)pyrazolo[5,1-b]thiazole-7-carboxamide

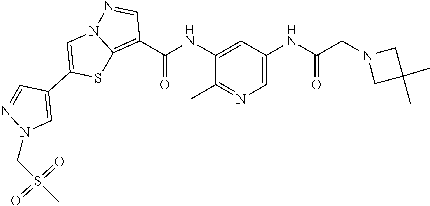 [SMILES: Cc1ncc(NC(=O)CN2CC(C)(C)C2)cc1NC(=O)c1cnn2cc(-c3cnn(CS(C)(=O)=O)c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 1-((methylsulfonyl)methyl)-4-(4,4,5,5-tetramethyl-1,3,2-dioxaborolan-2-yl)-1H-pyrazole (71.93 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. For 17.5 h. The reaction was diluted with MeOH (15 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min then 30% MeOH/CH₂Cl₂ over 5 min to afford the product, N-(5-(2-(3,3-dimethylazetidin-1-yl)acetamido)-2-methylpyridin-3-yl)-2-(1-((methylsulfonyl)methyl)-1H-pyrazol-4-yl)pyrazolo[5,1-b]thiazole-7-carboxamide, as a brown solid (42.9 mg). LCMS (ESI): mass calcd. for C₂₄H₂₈N₈O₄S₂, 556.2; m/z found, 557.3 [M+H]⁺; ¹H NMR (400 MHz, DMSO-d6) δ ppm 1.22 (s, 6H), 2.40 (s, 3H), 3.08 (s, 3H), 3.23 (br s, 4H), 3.45 (br s, 2H), 5.79 (s, 2H), 8.13 (s, 2H), 8.37 (s, 1H), 8.57 (s, 2H), 8.73 (s, 1H), 9.93 (s, 1H), 10.03 (br s, 1H).

### Examples 266: 2-(1-(cyanomethyl)-1H-pyrazol-4-yl)-N-(5-(2-(3,3-dimethylazetidin-1-yl)acetamido)-2-methylpyridin-3-yl)pyrazolo[5,1-b]thiazole-7-carboxamide and Example 267: 2-(1-(2-amino-2-oxoethyl)-1H-pyrazol-4-yl)-N-(5-(2-(3,3-dimethylazetidin-1-yl)acetamido)-2-methylpyridin-3-yl)pyrazolo[5,1-b]thiazole-7-carboxamide

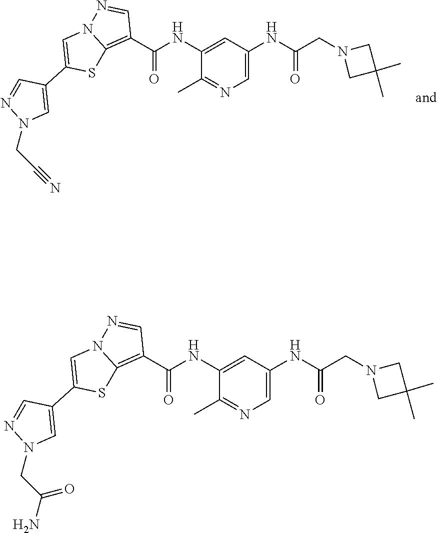 [SMILES: Cc1ncc(NC(=O)CN2CC(C)(C)C2)cc1NC(=O)c1cnn2cc(-c3cnn(CC#N)c3)sc12 ; Cc1ncc(NC(=O)CN2CC(C)(C)C2)cc1NC(=O)c1cnn2cc(-c3cnn(CC(N)=O)c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (150 mg, 0.31 mmol), 2-(4-(4,4,5,5-tetramethyl-1,3,2-dioxaborolan-2-yl)-1H-pyrazol-1-yl)acetonitrile (87.88 mg, 0.38 mmol) and K₂CO₃ (130.28 mg, 0.94 mmol) in 1,4-dioxane (2.5 mL) and water (0.5 mL) was added 1,1′-bis(diphenylphosphino)ferrocene-palladium(II)dichloride dichloromethane complex (51.32 mg, 0.063 mmol). The reaction mixture was thoroughly flushed with argon before being capped and heated at 100° C. for 18 h. The reaction was diluted with MeOH (15 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 minutes to afford the products, 2-(1-(cyanomethyl)-1H-pyrazol-4-yl)-N-(5-(2-(3,3-dimethylazetidin-1-yl)acetamido)-2-methylpyridin-3-yl)pyrazolo[5,1-b]thiazole-7-carboxamide as a brown brown solid, 31.1 mg; LCMS (ESI): mass calcd. for C₂₄H₂₅N₉O₂S, 503.2; m/z found, 504.3 [M+H]⁺; ¹H NMR (400 MHz, DMSO-d6) δ ppm 1.23 (s, 6H), 2.41 (s, 3H), 3.26 (br s, 4H), 3.53 (br s, 2H), 5.57 (s, 2H), 8.08 (s, 1H), 8.13 (d, J=1.00 Hz, 1H), 8.37 (s, 1H), 8.57 (d, J=1.00 Hz, 1H), 8.58 (s, 1H), 8.69 (s, 1H), 9.95 (s, 1H), 10.10 (br s, 1H), and 2-(1-(2-amino-2-oxoethyl)-1H-pyrazol-4-yl)-N-(5-(2-(3,3-dimethylazetidin-1-yl)acetamido)-2-methylpyridin-3-yl)pyrazolo[5,1-b]thiazole-7-carboxamide as a brown solid, 17.7 mg; LCMS (ESI): mass calcd. for C₂₄H₂₇N₉O₃S, 521.2; m/z found, 522.3 [M+H]⁺; ¹H NMR (400 MHz, DMSO-d6) δ ppm 1.22 (s, 6H), 2.41 (s, 3H), 3.13 (s, 4H), 6.22 (s, 2H), 7.40 (s, 2H), 7.69 (s, 2H), 7.90 (s, 1H), 8.13 (d, J=1.00 Hz, 1H), 8.20 (s, 1H), 8.54 (s, 1H), 8.57 (d, J=1.00 Hz, 1H), 8.62 (s, 1H), 9.94 (s, 1H), 9.96 (s, 1H)], respectively.

### Example 268: N-(5-(2-(3,3-dimethylazetidin-1-yl)acetamido)-2-methylpyridin-3-yl)-2-(1-(2,2,2-trifluoroethyl)-1H-pyrazol-4-yl)pyrazolo[5,1-b]thiazole-7-carboxamide

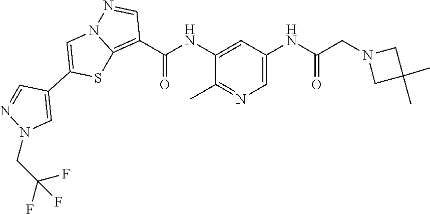 [SMILES: Cc1ncc(NC(=O)CN2CC(C)(C)C2)cc1NC(=O)c1cnn2cc(-c3cnn(CC(F)(F)F)c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 4-(4,4,5,5-tetramethyl-1,3,2-dioxaborolan-2-yl)-1-(2,2,2-trifluoroethyl)-1H-pyrazole (69.395 mg, 0.251 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 18.5 h. The reaction was diluted with MeOH (15 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min then 30% MeOH/CH₂Cl₂ over 5 min to afford the product, N-(5-(2-(3,3-dimethylazetidin-1-yl)acetamido)-2-methylpyridin-3-yl)-2-(1-(2,2,2-trifluoroethyl)-1H-pyrazol-4-yl)pyrazolo[5,1-b]thiazole-7-carboxamide, as a brown solid (44.6 mg). LCMS (ESI): mass calcd. for C₂₄H₂₅F₃N₈O₂S, 546.2; m/z found, 547.2 [M+H]⁺; ¹H NMR (400 MHz, DMSO-d6) δ ppm 1.22 (s, 6H), 2.41 (s, 3H), 3.20 (br s, 4H), 3.42 (br s, 2H), 5.21 (q, J=1.00 Hz, 2H), 8.07 (s, 1H), 8.14 (d, J=1.00 Hz, 1H), 8.37 (s, 1H), 8.56 (s, 2H), 8.70 (s, 1H), 9.93 (s, 1H), 10.02 (br s, 1H); ¹⁹F NMR (376 MHz, DMSO-d6) δ ppm −69.91 (s, 3F).

### Example 269: N-(5-(2-(3,3-dimethylazetidin-1-yl)acetamido)-2-methylpyridin-3-yl)-2-(1-(trifluoromethyl)-1H-pyrazol-4-yl)pyrazolo[5,1-b]thiazole-7-carboxamide

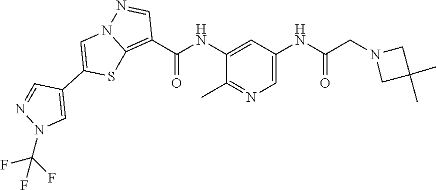 [SMILES: Cc1ncc(NC(=O)CN2CC(C)(C)C2)cc1NC(=O)c1cnn2cc(-c3cnn(C(F)(F)F)c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 4-(4,4,5,5-tetramethyl-1,3,2-dioxaborolan-2-yl)-1-(trifluoromethyl)-1H-pyrazole (65.87 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 21.5 h. The reaction was diluted with MeOH (15 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min then 30% MeOH/CH₂Cl₂ over 5 min to afford the product, N-(5-(2-(3,3-dimethylazetidin-1-yl)acetamido)-2-methylpyridin-3-yl)-2-(1-(trifluoromethyl)-1H-pyrazol-4-yl)pyrazolo[5,1-b]thiazole-7-carboxamide, as a brown solid (32.4 mg). LCMS (ESI): mass calcd. for C₂₃H₂₃F₃N₈O₂S, 532.2; m/z found, 533.3 [M+H]⁺; ¹H NMR (400 MHz, DMSO-d6) δ ppm 1.22 (s, 6H), 2.39 (s, 3H), 3.11 (br s, 4H), 3.19 (d, J=1.00 Hz, 2H), 8.12 (d, J=1.00 Hz, 1H), 8.49 (s, 1H), 8.56 (s, 1H), 8.57 (s, 1H), 8.83 (s, 1H), 9.08 (s, 1H), 9.89 (br s, 1H), 9.95 (s, 1H); ¹⁹F NMR (376 MHz, DMSO-d6) δ ppm −59.59 (s, 1F).

### Example 270: 2-(1-(cyclopropylmethyl)-1H-pyrazol-4-yl)-N-(5-(2-(3,3-dimethylazetidin-1-yl)acetamido)-2-methylpyridin-3-yl)pyrazolo[5,1-b]thiazole-7-carboxamide

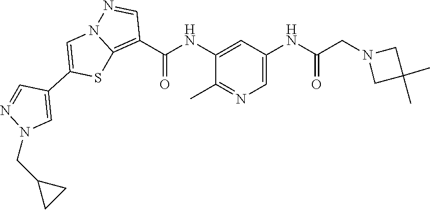 [SMILES: Cc1ncc(NC(=O)CN2CC(C)(C)C2)cc1NC(=O)c1cnn2cc(-c3cnn(CC4CC4)c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 1-(cyclopropylmethyl)-4-(4,4,5,5-tetramethyl-1,3,2-dioxaborolan-2-yl)-1H-pyrazole (62.37 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 22 h. The reaction was diluted with MeOH (15 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min then 30% MeOH/CH₂Cl₂ over 5 min to afford the product, 2-(1-(cyclopropylmethyl)-1H-pyrazol-4-yl)-N-(5-(2-(3,3-dimethylazetidin-1-yl)acetamido)-2-methylpyridin-3-yl)pyrazolo[5,1-b]thiazole-7-carboxamide, as a brown solid (53.0 mg). LCMS (ESI): mass calcd. for C₂₆H₃₀N₈O₂S, 518.2; m/z found, 519.3 [M+H]⁺; ¹H NMR (400 MHz, DMSO-d6) δ ppm 0.36-0.43 (m, 2H), 0.51-0.59 (m, 2H), 1.21 (s, 6H), 2.41 (s, 3H), 3.14-3.23 (m, 4H), 3.32 (br s, 3H), 4.01 (d, J=1.00 Hz, 2H), 7.89 (s, 1H), 8.13 (d, J=1.00 Hz, 1H), 8.27 (s, 1H), 8.53 (s, 1H), 8.56 (d, J=1.00 Hz, 1H), 8.59 (s, 1H), 9.89 (s, 1H), 9.97 (br s, 1H).

### Example 271: N-(5-(2-(3,3-dimethylazetidin-1-yl)acetamido)-2-methylpyridin-3-yl)-2-(1H-pyrazol-4-yl)pyrazolo[5,1-b]thiazole-7-carboxamide

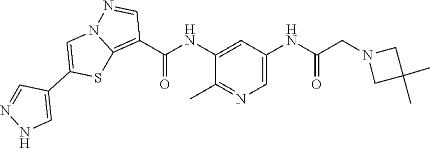 [SMILES: Cc1ncc(NC(=O)CN2CC(C)(C)C2)cc1NC(=O)c1cnn2cc(-c3cn[nH]c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 4-(4,4,5,5-tetramethyl-1,3,2-dioxaborolan-2-yl)-1H-pyrazole-1-carboxamide (59.59 mg, 0.25 mmol), and K₂CO₃ (86.85 mg, 0.63 mmol) in 1,4-dioxane (2.5 mL) and water (0.5 mLl) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 24 h. The reaction was diluted with MeOH (15 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min then 30% MeOH/CH₂Cl₂ over 5 min to afford the product, N-(5-(2-(3,3-dimethylazetidin-1-yl)acetamido)-2-methylpyridin-3-yl)-2-(1H-pyrazol-4-yl)pyrazolo[5,1-b]thiazole-7-carboxamide, as a brown solid (14.2 mg). LCMS (ESI): mass calcd. for C₂₂H₂₄N₈O₂S, 464.2; m/z found, 465.3 [M+H]⁺; ¹H NMR (400 MHz, DMSO-d6) δ ppm 1.22 (s, 6H), 2.41 (s, 3H), 3.15 (d, J=1.00 Hz, 2H), 3.23 (br s, 2H), 3.36-3.49 (m, 2H), 7.89-8.00 (m, 1H), 8.12 (d, J=1.00 Hz, 1H), 8.26 (br s, 1H), 8.55 (s, 1H), 8.57 (d, J=1.00 Hz, 1H), 8.61 (s, 1H), 9.93 (s, 1H), 10.05 (br s, 1H), 13.25 (br s, 1H).

### Example 272: N-(5-(2-(3,3-dimethylazetidin-1-yl)acetamido)-2-methylpyridin-3-yl)-2-(4-(3-hydroxyoxetan-3-yl)phenyl)pyrazolo[5,1-b]thiazole-7-carboxamide

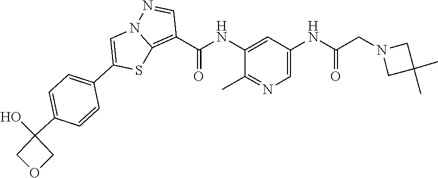 [SMILES: Cc1ncc(NC(=O)CN2CC(C)(C)C2)cc1NC(=O)c1cnn2cc(-c3ccc(C4(O)COC4)cc3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 3-(4-(4,4,5,5-tetramethyl-1,3,2-dioxaborolan-2-yl)phenyl)oxetan-3-ol (69.41 mg, 0.25 mmol) and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 19 h. The reaction was diluted with MeOH (25 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min then 30% MeOH/CH₂Cl₂ over 5 min to afford the product. LCMS indicated some impurity. The product was diluted with MeOH (25 mL) and silica gel (3 g) was added. All solvents were removed in vacuo. The silica gel mesh was loaded on a Redi Sep Rf silica gel cartridge (4 g) eluting with MeOH/EtOAc (15-30%) over 15 min to afford the product. The product was still not pure by LCMS, it was dissolved in DMSO (1 mL) and was further purified by HPLC (H₂O/ACN, 0.16% NH₄OH, XBridge 19×100) to afford the product, N-(5-(2-(3,3-dimethylazetidin-1-yl)acetamido)-2-methylpyridin-3-yl)-2-(4-(3-hydroxyoxetan-3-yl)phenyl)pyrazolo[5,1-b]thiazole-7-carboxamide, as a tan solid (21.9 mg). LCMS (ESI): mass calcd. for C₂₈H₃₀N₆O₄S, 546.20; m/z found, 547.2 [M+H]⁺; ¹H NMR (400 MHz, DMSO-d6) δ ppm 1.26 (s, 3H), 1.35 (s, 3H), 2.43 (s, 3H), 3.94 (d, J=1.00 Hz, 4H), 4.30 (d, J=1.00 Hz, 2H), 4.69 (d, J=1.00 Hz, 2H), 4.81 (d, J=1.00 Hz, 2H), 7.71 (d, J=1.00 Hz, 2H), 7.80 (d, J=1.00 Hz, 2H), 8.17 (d, J=1.00 Hz, 1H), 8.51 (d, J=1.00 Hz, 1H), 8.61 (s, 1H), 8.97 (s, 1H), 9.91 (s, 1H), 10.37-10.54 (m, 1H), 10.73 (s, 1H).

### Example 273: 2-(2-acetamidopyridin-4-yl)-N-(5-(2-(3,3-dimethylazetidin-1-yl)acetamido)-2-methylpyridin-3-yl)pyrazolo[5,1-b]thiazole-7-carboxamide

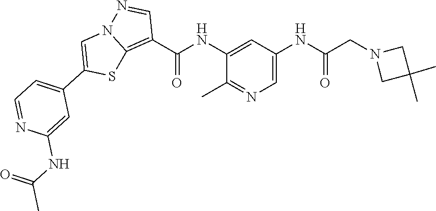 [SMILES: CC(=O)Nc1cc(-c2cn3ncc(C(=O)Nc4cc(NC(=O)CN5CC(C)(C)C5)cnc4C)c3s2)ccn1]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), N-(4-(3,3,4,4-tetramethyl-113,2,5-bromadioxolan-1-yl)pyridin-2-yl)acetamide (83.26 mg, 0.25 mmol) and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 19 h. The reaction was diluted with MeOH (25 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min then 30% MeOH/CH₂Cl₂ over 5 min to afford the product. LCMS indicated some impurity. The product was diluted with MeOH (25 mL) and silica gel (3 g) was added. All solvents were removed in vacuo. The silica gel mesh was loaded on a Redi Sep Rf silica gel cartridge (4 g) eluting with MeOH/EtOAc (15-30%) over 15 min to afford the product. The product was still not pure by LCMS, it was dissolved in DMSO (1 mL) and was further purified by HPLC (H₂O/ACN, 0.16% NH₄OH, XBridge 19×100) to afford the product, 2-(2-acetamidopyridin-4-yl)-N-(5-(2-(3,3-dimethylazetidin-1-yl)acetamido)-2-methylpyridin-3-yl)pyrazolo[5,1-b]thiazole-7-carboxamide, as a tan solid (20.6 mg). LCMS (ESI): mass calcd. for C₂₆H₂₈N₈O₃S, 532.2; m/z found, 533.3 [M+H]⁺; ¹H NMR (400 MHz, DMSO-d₆) δ ppm 1.25 (s, 3H), 1.36 (s, 3H), 2.14 (s, 3H), 2.45 (s, 3H), 2.55 (s, 2H), 3.94 (d, J=1.00 Hz, 4H), 7.51-7.62 (m, 1H), 8.21 (d, J=1.00 Hz, 1H), 8.35 (br s, 1H), 8.42 (d, J=1.00 Hz, 1H), 8.55 (d, J=1.00 Hz, 1H), 8.65 (s, 1H), 9.25 (s, 1H), 9.99 (s, 1H), 10.71 (s, 1H), 10.79 (s, 1H).

### Example 274: N-(5-(2-(3,3-dimethylazetidin-1-yl)acetamido)-2-methylpyridin-3-yl)-2-(2-(methylcarbamoyl)pyridin-4-yl)pyrazolo[5,1-b]thiazole-7-carboxamide

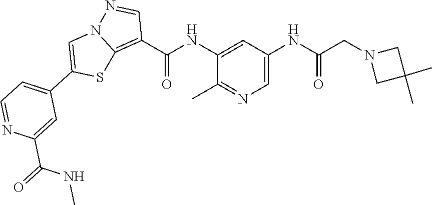 [SMILES: CNC(=O)c1cc(-c2cn3ncc(C(=O)Nc4cc(NC(=O)CN5CC(C)(C)C5)cnc4C)c3s2)ccn1]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), N-methyl-4-(3,3,4,4-tetramethyl-113,2,5-bromadioxolan-1-yl)picolinamide (83.25 mg, 0.25 mmol) and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 19 h. The reaction was diluted with MeOH (25 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min then 30% MeOH/CH₂Cl₂ over 5 min to afford the product. LCMS indicated some impurity. The product was diluted with MeOH (25 mL) and silica gel (3 g) was added. All solvents were removed in vacuo. The silica gel mesh was loaded on a Redi Sep Rf silica gel cartridge (4 g) eluting with MeOH/EtOAc (15-30%) over 15 min to afford the product. The product was still not pure by LCMS, it was dissolved in DMSO (1 mL) and was further purified by HPLC (H₂O/ACN, 0.16% NH₄OH, XBridge 19×100) to afford the product, N-(5-(2-(3,3-dimethylazetidin-1-yl)acetamido)-2-methylpyridin-3-yl)-2-(2-(methylcarbamoyl)pyridin-4-yl)pyrazolo[5,1-b]thiazole-7-carboxamide, as a tan solid (15.9 mg). LCMS (ESI): mass calcd. for C₂₆H₂₈N₈O₃S, 532.2; m/z found, 533.1 [M+H]⁺; ¹H NMR (400 MHz, DMSO-d₆) δ ppm 1.25 (s, 3H), 1.36 (s, 3H), 2.44 (s, 4H), 2.84 (d, J=1.00 Hz, 3H), 3.94 (d, J=1.00 Hz, 4H), 7.94 (dd, J=1.00 Hz, 1H), 8.16 (d, J=1.00 Hz, 1H), 8.29 (d, J=1.00 Hz, 1H), 8.55 (d, J=1.00 Hz, 1H), 8.66 (s, 1H), 8.72 (d, J=1.00 Hz, 1H), 8.83-8.91 (m, 1H), 9.45 (s, 1H), 9.95 (s, 1H), 10.45 (br s, 1H), 10.72 (s, 1H).

### Example 275: N-(5-(2-(3,3-dimethylazetidin-1-yl)acetamido)-2-methylpyridin-3-yl)-2-(1-(methylsulfonyl)-1H-pyrrol-3-yl)pyrazolo[5,1-b]thiazole-7-carboxamide

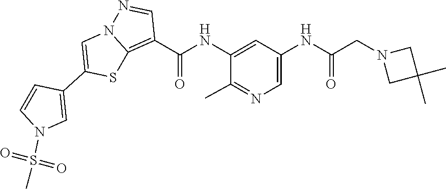 [SMILES: Cc1ncc(NC(=O)CN2CC(C)(C)C2)cc1NC(=O)c1cnn2cc(-c3ccn(S(C)(=O)=O)c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 1-(methylsulfonyl)-3-(4,4,5,5-tetramethyl-1,3,2-dioxaborolan-2-yl)-1H-pyrrole (68.16 mg, 0.25 mmol) and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 19.5 h. The reaction was diluted with MeOH (25 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min then 30% MeOH/CH₂Cl₂ over 5 min to afford the product. LCMS indicated some impurity. The product was diluted with MeOH (25 mL) and silica gel (3 g) was added. All solvents were removed in vacuo. The silica gel mesh was loaded on a Redi Sep Rf silica gel cartridge (4 g) eluting with MeOH/EtOAc (15-30%) over 15 min to afford the product. The product was still not pure by LCMS, it was dissolved in DMSO (1 mL) and submitted to the purification group for isolation to afford the product, N-(5-(2-(3,3-dimethylazetidin-1-yl)acetamido)-2-methylpyridin-3-yl)-2-(1-(methylsulfonyl)-1H-pyrrol-3-yl)pyrazolo[5,1-b]thiazole-7-carboxamide, as a tan solid (31.6 mg). LCMS (ESI): mass calcd. for C₂₄H₂₇N704S₂, 541.16; m/z found, 542.1 [M+H]⁺;

### Example 277: 2-(5-acetamidopyridin-3-yl)-N-(5-(2-(3,3-dimethylazetidin-1-yl)acetamido)-2-methylpyridin-3-yl)pyrazolo[5,1-b]thiazole-7-carboxamide

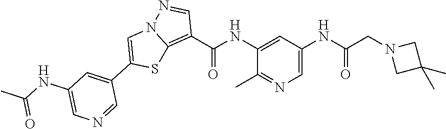 [SMILES: CC(=O)Nc1cncc(-c2cn3ncc(C(=O)Nc4cc(NC(=O)CN5CC(C)(C)C5)cnc4C)c3s2)c1]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), N-(5-(4,4,5,5-tetramethyl-1,3,2-dioxaborolan-2-yl)pyridin-3-yl)acetamide (65.89 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction was thoroughly flushed with argon before being capped and heated at 100° C. for 22 h. The reaction was diluted with MeOH (25 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min then 30% MeOH/CH₂Cl₂ over 5 min to afford the product. LCMS indicated some impurity. The product was diluted with MeOH (25 mL) and silica gel (3 g) was added. All solvents were removed in vacuo. The silica gel mesh was loaded on a Redi Sep Rf silica gel cartridge (4 g) eluting with MeOH/EtOAc (15-30%) over 15 min to afford the product. The product was still not pure by LCMS, it was dissolved in DMSO (1 mL) and submitted to the purification group for isolation to afford the product, 2-(5-acetamidopyridin-3-yl)-N-(5-(2-(3,3-dimethylazetidin-1-yl)acetamido)-2-methylpyridin-3-yl)pyrazolo[5,1-b]thiazole-7-carboxamide, as a tan solid (20.8 mg). LCMS (ESI): mass calcd. for C₂₆H₂₈N₈O₃S, 532.2; m/z found, 533.1 [M+H]⁺; ¹H NMR (400 MHz, DMSO-d₆) δ ppm 1.23 (s, 3H), 1.36 (s, 3H), 2.11 (s, 3H), 2.46 (s, 4H), 3.93 (d, J=1.00 Hz, 4H), 8.16 (s, 1H), 8.36 (s, 1H), 8.54 (d, J=1.00 Hz, 1H), 8.62 (s, 1H), 8.67 (d, J=1.00 Hz, 1H), 8.73 (d, J=1.00 Hz, 1H), 9.06 (s, 1H), 9.93 (s, 1H), 10.36 (s, 1H), 10.46 (br s, 1H), 10.73 (s, 1H).

### Example 278: N-(5-(2-(3,3-dimethylazetidin-1-yl)acetamido)-2-methylpyridin-3-yl)-2-(5-(hydroxymethyl)thiophen-2-yl)pyrazolo[5,1-b]thiazole-7-carboxamide

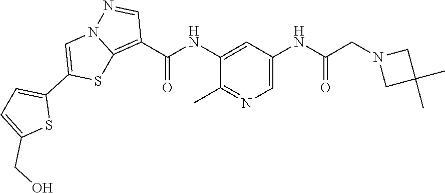 [SMILES: Cc1ncc(NC(=O)CN2CC(C)(C)C2)cc1NC(=O)c1cnn2cc(-c3ccc(CO)s3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), (5-(4,4,5,5-tetramethyl-1,3,2-dioxaborolan-2-yl)thiophen-2-yl)methanol (60.36 mg, 0.25 mmol)), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction was thoroughly flushed with argon before being capped and heated at 100° C. 18.5 h. The reaction was diluted with MeOH (25 mL) and silica gel (3 g) was added. All solvents were removed in vacuo. The silica gel mesh was loaded on a Redi Sep Rf silica gel cartridge (25 g) eluting with MeOH/CH₂Cl₂ (0-30%) over 15 min to afford the product, N-(5-(2-(3,3-dimethylazetidin-1-yl)acetamido)-2-methylpyridin-3-yl)-2-(5-(hydroxymethyl)thiophen-2-yl)pyrazolo[5,1-b]thiazole-7-carboxamide, as an off-white solid (3.75 mg). LCMS (ESI): mass calcd. for C₂₄H₂₆N₆O₃S₂, 510.2; m/z found, 511.0 [M+H]⁺; ¹H NMR (400 MHz, DMSO-d6) δ ppm 1.20 (s, 6H), 2.40 (s, 3H), 3.04 (s, 4H), 3.22 (s, 2H), 4.66 (d, J=1.00 Hz, 2H), 5.63 (t, J=1.00 Hz, 1H), 6.97 (d, J=1.00 Hz, 1H), 7.32 (d, J=1.00 Hz, 1H), 8.15 (d, J=1.00 Hz, 1H), 8.54 (s, 1H), 8.56 (d, J=1.00 Hz, 1H), 8.75 (s, 1H), 9.82 (s, 1H), 9.91 (s, 1H).

### Example 279: N-(5-(2-(3,3-dimethylazetidin-1-yl)acetamido)-2-methylpyridin-3-yl)-2-(1-(2-(methylamino)-2-oxoethyl)-1H-pyrazol-4-yl)pyrazolo[5,1-b]thiazole-7-carboxamide

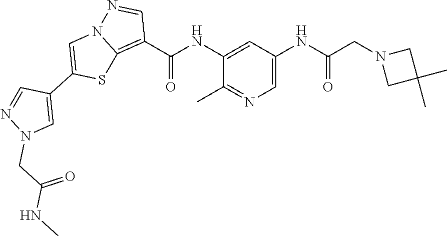 [SMILES: CNC(=O)Cn1cc(-c2cn3ncc(C(=O)Nc4cc(NC(=O)CN5CC(C)(C)C5)cnc4C)c3s2)cn1]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), N-methyl-2-(4-(4,4,5,5-tetramethyl-1,3,2-dioxaborolan-2-yl)-1H-pyrazol-1-yl)acetamide (66.64 mg, 0.25 mmol) and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction was thoroughly flushed with argon before being capped and heated at 100° C. for 15.5 h. The reaction was diluted with MeOH (25 mL) and silica gel (3 g) was added. All solvents were removed in vacuo. The silica gel mesh was loaded on a Redi Sep Rf silica gel cartridge (25 g) eluting with MeOH/CH₂Cl₂ (0-30%) over 15 min to afford the product, N-(5-(2-(3,3-dimethylazetidin-1-yl)acetamido)-2-methylpyridin-3-yl)-2-(1-(2-(methylamino)-2-oxoethyl)-1H-pyrazol-4-yl)pyrazolo[5,1-b]thiazole-7-carboxamide, as an off-white solid (25.85 mg). LCMS (ESI): mass calcd. for C₂₅H₂₉N₉O₃S, 535.2; m/z found, 536.1 [M+H]⁺; ¹H NMR (400 MHz, DMSO-d6) δ ppm 1.21 (s, 6H), 2.40 (s, 3H), 2.63 (d, J=1.00 Hz, 3H), 3.08 (br s, 4H), 3.24 (br s, 2H), 4.81 (s, 2H), 7.91 (s, 1H), 8.03-8.11 (m, 1H), 8.14 (d, J=1.00 Hz, 1H), 8.21 (s, 1H), 8.51 (s, 1H), 8.56 (d, J=1.00 Hz, 1H), 8.62 (s, 1H), 9.83 (br s, 1H), 9.88 (s, 1H).

### Example 280: N-(5-(2-(3,3-dimethylazetidin-1-yl)acetamido)-2-methylpyridin-3-yl)-2-(1-(1,1-dioxidotetrahydro-2H-thiopyran-4-yl)-1H-pyrazol-4-yl)pyrazolo[5,1-b]thiazole-7-carboxamide

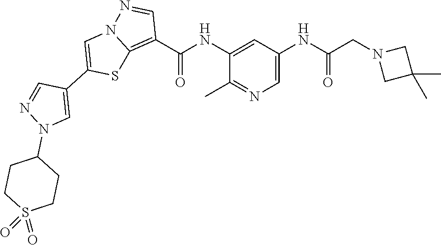 [SMILES: Cc1ncc(NC(=O)CN2CC(C)(C)C2)cc1NC(=O)c1cnn2cc(-c3cnn(C4CCS(=O)(=O)CC4)c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 4-(4-(4,4,5,5-tetramethyl-1,3,2-dioxaborolan-2-yl)-1H-pyrazol-1-yl)tetrahydro-2H-thiopyran 1,1-dioxide (82 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 19 h. The reaction was diluted with MeOH (25 mL) and silica gel (3 g) was added. All solvents were removed in vacuo. The silica gel mesh was loaded on a Redi Sep Rf silica gel cartridge (25 g) eluting with MeOH/CH₂Cl₂ (0-30%) over 15 min to afford a grey solid. The solid was further purified by HPLC (H₂O/ACN, 0.16% NH₄OH, XBridge 19×100) to afford the product, N-(5-(2-(3,3-dimethylazetidin-1-yl)acetamido)-2-methylpyridin-3-yl)-2-(1-(1,1-dioxidotetrahydro-2H-thiopyran-4-yl)-1H-pyrazol-4-yl)pyrazolo[5,1-b]thiazole-7-carboxamide, as an off-white solid (12.04 mg). LCMS (ESI): mass calcd. for C₂₇H₃₂N₈O₄S₂, 596.2; m/z found, 597.3 [M+H]⁺; ¹H NMR (400 MHz, DMSO-d₆) δ ppm 1.21 (s, 6H), 1.79-1.97 (m, 1H), 1.97-2.08 (m, 1H), 2.08-2.22 (m, 2H), 2.40 (s, 3H), 2.56 (s, 2H), 3.06 (br s, 2H), 3.11-3.22 (m, 2H), 3.22-3.29 (m, 2H), 3.53-3.62 (m, 1H), 3.62-3.74 (m, 1H), 4.72 (tt, J=1.00 Hz, 1H), 7.98 (s, 1H), 8.14 (d, J=1.00 Hz, 1H), 8.36 (s, 1H), 8.51 (s, 1H), 8.56 (d, J=1.00 Hz, 1H), 8.61 (s, 1H), 9.82 (br s, 1H), 9.89 (s, 1H).

### Example 281: N-(5-(2-(3,3-dimethylazetidin-1-yl)acetamido)-2-methylpyridin-3-yl)-2-(1-(2-hydroxypropyl)-1H-pyrazol-4-yl)pyrazolo[5,1-b]thiazole-7-carboxamide

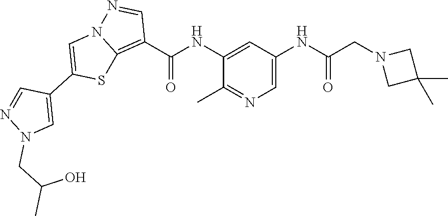 [SMILES: Cc1ncc(NC(=O)CN2CC(C)(C)C2)cc1NC(=O)c1cnn2cc(-c3cnn(CC(C)O)c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 1-[4-(tetramethyl-1,3,2-dioxaborolan-2-yl)-1H-pyrazol-1-yl]propan-2-ol (63.38 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 22 h. All solvents were removed in vacuo. The reaction was diluted with MeOH (25 mL) and silica gel (5 g) was added. All solvents were removed in vacuo. The silica gel mesh was loaded on a Redi Sep Rf silica gel cartridge (24 g) eluting with 10% NH₄OH/MeOH and CH₂Cl₂ (0-30%) over 15 min to afford a dark solid. The solid was dissolved in MeOH (25 mL) and silica gel (5 g). All solvents were removed in vacuo. The silica gel mesh was loaded on a Redi Sep Rf silica gel cartridge (24 g) eluting with MeOH and EtOAc (15-30%) over 15 min to afford the product, N-(5-(2-(3,3-dimethylazetidin-1-yl)acetamido)-2-methylpyridin-3-yl)-2-(1-(2-hydroxypropyl)-1H-pyrazol-4-yl)pyrazolo[5,1-b]thiazole-7-carboxamide, as a grey solid (15.6 mg). LCMS (ESI): mass calcd. for C₂₅H₃₀N₈O₃S, 522.2; m/z found, 523.3 [M+H]⁺; ¹H NMR (400 MHz, DMSO-d6) δ ppm 1.05 (d, J=1.00 Hz, 3H), 1.20 (s, 6H), 2.40 (s, 3H), 3.06 (br s, 4H), 3.25 (br s, 2H), 3.92-4.13 (m, 3H), 4.97 (d, J=1.00 Hz, 1H), 7.88 (s, 1H), 8.14 (d, J=1.00 Hz, 1H), 8.19 (s, 1H), 8.50 (s, 1H), 8.55 (d, J=1.00 Hz, 1H), 8.60 (s, 1H), 9.81 (s, 1H), 9.88 (s, 1H).

### Example 282: N-(5-(2-(3,3-dimethylazetidin-1-yl)acetamido)-2-methylpyridin-3-yl)-2-(thiophen-3-yl)pyrazolo[5,1-b]thiazole-7-carboxamide

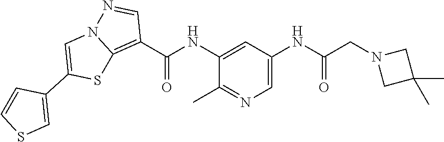 [SMILES: Cc1ncc(NC(=O)CN2CC(C)(C)C2)cc1NC(=O)c1cnn2cc(-c3ccsc3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), thiophene-3-boronic acid, pinacol ester (70.18 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 19 h. All solvents were removed in vacuo. The reaction was diluted with MeOH (25 mL) and silica gel (5 g) was added. All solvents were removed in vacuo. The silica gel mesh was loaded on a Redi Sep Rf silica gel cartridge (24 g) eluting with 10% NH₄OH/MeOH and CH₂Cl₂ (0-30%) over 15 min to afford a dark solid. The solid was dissolved in MeOH (25 mL) and silica gel (5 g). All solvents were removed in vacuo. The silica gel mesh was loaded on a Redi Sep Rf silica gel cartridge (24 g) eluting with MeOH and EtOAc (15-30%) over 15 min to afford the product, N-(5-(2-(3,3-dimethylazetidin-1-yl)acetamido)-2-methylpyridin-3-yl)-2-(thiophen-3-yl)pyrazolo[5,1-b]thiazole-7-carboxamide, as a grey solid (12.16 mg). LCMS (ESI): mass calcd. for C₂₃H₂₄N₆O₂S₂, 480.1; m/z found, 481.2 [M+H]⁺; ¹H NMR (400 MHz, DMSO-d6) δ ppm 1.14 (s, 6H), 2.39 (s, 3H), 3.05 (br s, 4H), 3.23 (br s, 2H), 7.59 (dd, J=1.00 Hz, 1H), 7.74 (dd, J=1.00 Hz, 1H), 7.95 (dd, J=1.00 Hz, 1H), 8.14 (d, J=1.00 Hz, 1H), 8.54 (s, 1H), 8.56 (d, J=1.00 Hz, 1H), 8.82 (s, 1H), 9.83 (s, 1H), 9.92 (s, 1H).

### Example 283: N-(5-(2-(3,3-dimethylazetidin-1-yl)acetamido)-2-methylpyridin-3-yl)-2-(furan-3-yl)pyrazolo[5,1-b]thiazole-7-carboxamide

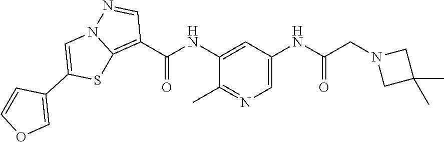 [SMILES: Cc1ncc(NC(=O)CN2CC(C)(C)C2)cc1NC(=O)c1cnn2cc(-c3ccoc3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), furan-3-boronic acid, pinacol ester (48.78 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 19 h. All solvents were removed in vacuo. The reaction was diluted with MeOH (25 mL) and silica gel (5 g) was added. All solvents were removed in vacuo. The silica gel mesh was loaded on a Redi Sep Rf silica gel cartridge (24 g) eluting with 10% NH₄OH/MeOH and CH₂Cl₂ (0-30%) over 15 min to afford a dark solid. The solid was dissolved in MeOH (25 mL) and silica gel (5 g). All solvents were removed in vacuo. The silica gel mesh was loaded on a Redi Sep Rf silica gel cartridge (24 g) eluting with MeOH and EtOAc (15-30%) over 15 min to afford the product, N-(5-(2-(3,3-dimethylazetidin-1-yl)acetamido)-2-methylpyridin-3-yl)-2-(furan-3-yl)pyrazolo[5,1-b]thiazole-7-carboxamide, as a grey solid (22.95 mg). LCMS (ESI): mass calcd. for C₂₃H₂₄N₆O₃S, 464.2; m/z found, 465.1 [M+H]⁺; ¹H NMR (400 MHz, DMSO-d6) δ ppm 1.19 (s, 6H), 2.38 (s, 3H), 3.08 (br s, 4H), 3.23 (br s, 2H), 7.00 (s, 1H), 7.81-7.85 (m, 1H), 8.13 (d, J=1.00 Hz, 1H), 8.26 (s, 1H), 8.52 (s, 1H), 8.56 (d, J=1.00 Hz, 1H), 8.72 (s, 1H), 9.82 (br s, 1H), 9.92 (br s, 1H).

### Example 284: N-(5-(2-(3,3-dimethylazetidin-1-yl)acetamido)-2-methylpyridin-3-yl)-2-(2-morpholinopyridin-4-yl)pyrazolo[5,1-b]thiazole-7-carboxamide

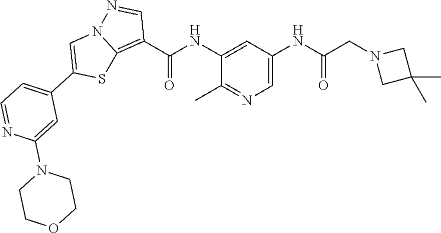 [SMILES: Cc1ncc(NC(=O)CN2CC(C)(C)C2)cc1NC(=O)c1cnn2cc(-c3ccnc(N4CCOCC4)c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 2-morpholinopyridine-4-boronic acid, pinacol ester (72.94 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.041 mmol). The reaction mixture was thoroughly flushed with argon before being capped and heated at 100° C. for 19 h. All solvents were removed in vacuo. The reaction was diluted with MeOH (25 mL) and silica gel (5 g) was added. All solvents were removed in vacuo. The silica gel mesh was loaded on a Redi Sep Rf silica gel cartridge (24 g) eluting with 10% NH₄OH/MeOH and CH₂Cl₂ (0-30%) over 15 min to afford a dark solid. The solid was dissolved in MeOH (25 mL) and silica gel (5 g). All solvents were removed in vacuo. The silica gel mesh was loaded on a Redi Sep Rf silica gel cartridge (24 g) eluting with MeOH and EtOAc (15-30%) over 15 min to afford the product, N-(5-(2-(3,3-dimethylazetidin-1-yl)acetamido)-2-methylpyridin-3-yl)-2-(2-morpholinopyridin-4-yl)pyrazolo[5,1-b]thiazole-7-carboxamide, as a grey solid (21.91 mg). LCMS (ESI): mass calcd. for C₂₈H₃₂N₈O₃S, 560.2; m/z found, 561.3 [M+H]⁺; ¹H NMR (400 MHz, DMSO-d6) δ ppm 1.19 (s, 6H), 2.39 (s, 3H), 3.07 (br s, 4H), 3.24 (br s, 2H), 3.45-3.59 (m, 4H), 3.66-3.79 (m, 4H), 7.00 (dd, J=1.00 Hz, 1H), 7.12 (s, 1H), 8.14 (d, J=1.00 Hz, 1H), 8.20 (d, J=1.00 Hz, 1H), 8.54 (d, J=1.00 Hz, 1H), 8.61 (s, 1H), 9.23 (s, 1H), 9.85 (br s, 1H), 9.96 (br s, 1H).

### Example 285: N-(5-(2-(3,3-dimethylazetidin-1-yl)acetamido)-2-methylpyridin-3-yl)-2-(1H-pyrrol-3-yl)pyrazolo[5,1-b]thiazole-7-carboxamide

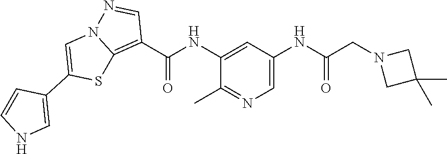 [SMILES: Cc1ncc(NC(=O)CN2CC(C)(C)C2)cc1NC(=O)c1cnn2cc(-c3cc[nH]c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), pyrrole-3-boronic acid, pinacol ester (48.53 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 19 h. All solvents were removed in vacuo. The reaction was diluted with MeOH (25 mL) and silica gel (5 g) was added. All solvents were removed in vacuo. The silica gel mesh was loaded on a Redi Sep Rf silica gel cartridge (24 g) eluting with 10% NH₄OH/MeOH and CH₂Cl₂ (0-30%) over 15 min to afford a dark solid. The solid was dissolved in MeOH (25 mL) and silica gel (5 g). All solvents were removed in vacuo. The silica gel mesh was loaded on a Redi Sep Rf silica gel cartridge (24 g) eluting with MeOH and EtOAc (15-30%) over 15 min to afford the product, N-(5-(2-(3,3-dimethylazetidin-1-yl)acetamido)-2-methylpyridin-3-yl)-2-(1H-pyrrol-3-yl)pyrazolo[5,1-b]thiazole-7-carboxamide, as a grey solid (16.49 mg). LCMS (ESI): mass calcd. for C₂₃H₂₅N₇O₂S, 463.2; m/z found, 464.2 [M+H]⁺; ¹H NMR (400 MHz, DMSO-d6) δ ppm 1.21 (s, 6H), 2.39 (s, 3H), 3.05 (s, 3H), 3.22 (s, 2H), 3.33 (s, 1H), 6.37-6.48 (m, 1H), 6.80-6.91 (m, 1H), 7.16-7.29 (m, 1H), 8.06-8.18 (m, 1H), 8.34-8.42 (m, 1H), 8.42-8.49 (m, 1H), 8.50-8.59 (m, 1H), 9.72-9.91 (m, 2H), 11.18 (br s, 1H).

### Example 286: N-(5-(2-(3,3-dimethylazetidin-1-yl)acetamido)-2-methylpyridin-3-yl)-2-(1-methyl-1H-indazol-4-yl)pyrazolo[5,1-b]thiazole-7-carboxamide

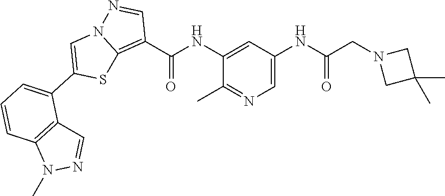 [SMILES: Cc1ncc(NC(=O)CN2CC(C)(C)C2)cc1NC(=O)c1cnn2cc(-c3cccc4c3cnn4C)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 1-methyl-1H-indazole-4-boronic acid pinacol ester (64.89 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 16 h. All solvents were removed in vacuo. The reaction was diluted with MeOH (25 mL) and silica gel (5 g) was added. All solvents were removed in vacuo. The silica gel mesh was loaded on a Redi Sep Rf silica gel cartridge (24 g) eluting with 10% NH₄OH/MeOH and CH₂Cl₂ (0-30%) over 15 min to afford a dark solid. The solid was dissolved in MeOH (25 mL) and silica gel (5 g). All solvents were removed in vacuo. The silica gel mesh was loaded on a Redi Sep Rf silica gel cartridge (24 g) eluting with MeOH and EtOAc (15-30%) over 15 min to afford the product, N-(5-(2-(3,3-dimethylazetidin-1-yl)acetamido)-2-methylpyridin-3-yl)-2-(1-methyl-1H-indazol-4-yl)pyrazolo[5,1-b]thiazole-7-carboxamide, as a grey solid (21.51 mg). LCMS (ESI): mass calcd. for C₂₇H₂₈N₈O₂S, 528.2; m/z found, 529.2 [M+H]⁺; ¹H NMR (400 MHz, DMSO-d6) δ ppm 1.21 (s, 6H), 2.41 (s, 3H), 3.11 (br s, 4H), 3.25-3.30 (m, 2H), 4.10 (s, 3H), 7.45 (d, J=1.00 Hz, 1H), 7.52 (t, J=1.00 Hz, 1H), 7.77 (d, J=1.00 Hz, 1H), 8.18 (d, J=1.00 Hz, 1H), 8.55 (s, 1H), 8.57 (d, J=1.00 Hz, 1H), 8.61 (s, 1H), 9.04 (s, 1H), 9.85 (br s, 1H), 9.95 (br s, 1H).

### Example 287: N-(5-(2-(3,3-dimethylazetidin-1-yl)acetamido)-2-methylpyridin-3-yl)-2-(1H-indol-3-yl)pyrazolo[5,1-b]thiazole-7-carboxamide

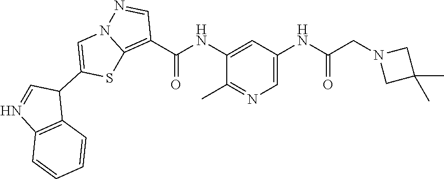 [SMILES: Cc1ncc(NC(=O)CN2CC(C)(C)C2)cc1NC(=O)c1cnn2cc(C3C=Nc4ccccc43)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), indole-3-boronic acid pinacol ester (61.11 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 16.5 h. All solvents were removed in vacuo. The reaction was diluted with MeOH (25 mL) and silica gel (5 g) was added. All solvents were removed in vacuo. The silica gel mesh was loaded on a Redi Sep Rf silica gel cartridge (24 g) eluting with 10% NH₄OH/MeOH and CH₂Cl₂ (0-30%) over 15 min to afford a dark solid. The solid was dissolved in MeOH (25 mL) and silica gel (5 g). All solvents were removed in vacuo. The silica gel mesh was loaded on a Redi Sep Rf silica gel cartridge (24 g) eluting with MeOH and EtOAc (15-30%) over 15 min to afford the product, N-(5-(2-(3,3-dimethylazetidin-1-yl)acetamido)-2-methylpyridin-3-yl)-2-(1H-indol-3-yl)pyrazolo[5,1-b]thiazole-7-carboxamide, as a grey solid (23.11 mg). LCMS (ESI): mass calcd. for C₂₇H₂₇N₇O₂S, 513.2; m/z found, 514.2 [M+H]⁺; ¹H NMR (400 MHz, DMSO-d6) δ ppm 1.20 (s, 6H), 2.42 (s, 3H), 3.08 (br s, 4H), 3.25 (br s, 2H), 7.17 (dt, J=1.00 Hz, 1H), 7.24 (dt, J=1.00 Hz, 1H), 7.48 (d, J=1.00 Hz, 1H), 7.90 (d, J=1.00 Hz, 1H), 7.97 (d, J=1.00 Hz, 1H), 8.16 (d, J=1.00 Hz, 1H), 8.52 (s, 1H), 8.56 (d, J=1.00 Hz, 1H), 8.65 (s, 1H), 9.86 (s, 2H), 11.68 (d, J=1.00 Hz, 1H).

### Example 288: N-(5-(2-(3,3-dimethylazetidin-1-yl)acetamido)-2-methylpyridin-3-yl)-2-(1H-pyrazolo[3,4-b]pyridin-5-yl)pyrazolo[5,1-b]thiazole-7-carboxamide

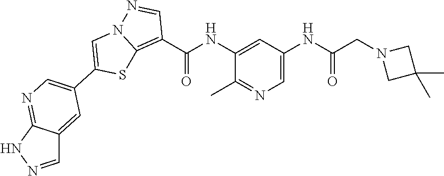 [SMILES: Cc1ncc(NC(=O)CN2CC(C)(C)C2)cc1NC(=O)c1cnn2cc(-c3cnc4[nH]ncc4c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 5-(4,4,5,5-tetramethyl-1,3,2-dioxaborolan-2-yl)-1H-pyrazolo[3,4-b]pyridine (61.61 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction mixture was thoroughly flushed with argon before being capped and heated at 100° C. for 19 h. The reaction was diluted with MeOH (25 mL) and silica gel (3 g) was added. All solvents were removed in vacuo. The silica gel mesh was loaded on a Redi Sep Rf silica gel cartridge (24 g) eluting with MeOH/CH₂Cl₂ (15-30%) over 15 min to afford the product. The product was diluted with MeOH (25 mL) and silica gel (1 g) was added. All solvents were removed in vacuo. The silica gel mesh was loaded on a Redi Sep Rf silica gel cartridge (24 g) eluting with MeOH/EtOAc (15-30%) over 15 min to afford the product, N-(5-(2-(3,3-dimethylazetidin-1-yl)acetamido)-2-methylpyridin-3-yl)-2-(1H-pyrazolo[3,4-b]pyridin-5-yl)pyrazolo[5,1-b]thiazole-7-carboxamide, as an off-white solid (6.7 mg). LCMS (ESI): mass calcd. for C₂₅H₂₅N₉O₂S, 515.2; m/z found, 516.2 [M+H]⁺; ¹H NMR (400 MHz, DMSO-d6) δ ppm 1.21 (s, 6H), 2.42 (s, 3H), 3.11 (br s, 4H), 3.27 (br s, 2H), 8.14-8.19 (m, 1H), 8.24 (br s, 1H), 8.56 (d, J=1.00 Hz, 2H), 8.59 (s, 1H), 8.99 (d, J=1.00 Hz, 1H), 9.03 (s, 1H), 9.85 (br s, 1H), 9.93 (s, 1H), 13.91 (br s, 1H).

### Example 289: N-(5-(2-(3,3-dimethylazetidin-1-yl)acetamido)-2-methylpyridin-3-yl)-2-(1-(pyridin-3-yl)-1H-pyrazol-4-yl)pyrazolo[5,1-b]thiazole-7-carboxamide

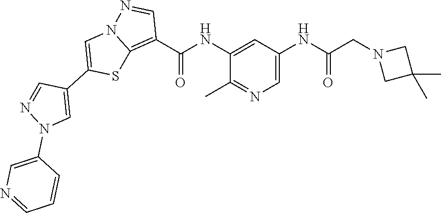 [SMILES: Cc1ncc(NC(=O)CN2CC(C)(C)C2)cc1NC(=O)c1cnn2cc(-c3cnn(-c4cccnc4)c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 3-[4-(tetramethyl-1,3,2-dioxaborolan-2-yl)-1H-pyrazol-1-yl]pyridine (68.15 mg, 0.21 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction was thoroughly flushed with argon before being capped and heated at 100° C. for 22 h. The reaction was diluted with MeOH (25 mL) and silica gel (5 g) was added. All solvents were removed in vacuo. The silica gel mesh was loaded on a Redi Sep Rf silica gel cartridge (40 g) eluting with MeOH and CH₂Cl₂ (0-30%) over 15 min to afford the product, N-(5-(2-(3,3-dimethylazetidin-1-yl)acetamido)-2-methylpyridin-3-yl)-2-(1-(pyridin-3-yl)-1H-pyrazol-4-yl)pyrazolo[5,1-b]thiazole-7-carboxamide, as a dark brown solid (6.9 mg). LCMS (ESI): mass calcd. for C₂₇H₂₇N₉O₂S, 541.2; m/z found, 542.3 [M+H]⁺; ¹H NMR (400 MHz, DMSO-d6) δ ppm 1.19 (s, 6H), 2.40 (s, 3H), 3.04 (s, 4H), 3.22 (s, 2H), 7.56-7.65 (m, 1H), 8.14 (d, J=1.00 Hz, 1H), 8.28 (d, J=1.00 Hz, 1H), 8.34 (s, 1H), 8.51-8.60 (m, 3H), 8.75 (s, 1H), 9.12 (s, 1H), 9.16 (d, J=1.00 Hz, 1H), 9.82 (s, 1H), 9.92 (s, 1H).

### Example 290: N-(5-(2-(3,3-dimethylazetidin-1-yl)acetamido)-2-methylpyridin-3-yl)-2-(1-(2-hydroxyethyl)-1H-pyrazol-4-yl)pyrazolo[5,1-b]thiazole-7-carboxamide

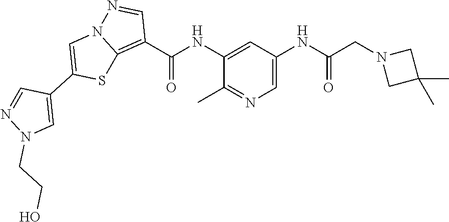 [SMILES: Cc1ncc(NC(=O)CN2CC(C)(C)C2)cc1NC(=O)c1cnn2cc(-c3cnn(CCO)c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 2-[4-(tetramethyl-1,3,2-dioxaborolan-2-yl)-1H-pyrazol-1-yl]ethan-1-ol (59.85 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction was thoroughly flushed with argon before being capped and heated at 100° C. for 24 h. The reaction was diluted with MeOH (25 mL) and silica gel (5 g) was added. All solvents were removed in vacuo. The silica gel mesh was loaded on a Redi Sep Rf silica gel cartridge (40 g) eluting with MeOH and CH₂Cl₂ (0-30%) over 15 min to afford the product. The product was dissolved in DMSO (1.5 mL) and was further purified by HPLC (H₂O/ACN, 0.16% NH₄OH, XBridge 19×100) to afford the product, N-(5-(2-(3,3-dimethylazetidin-1-yl)acetamido)-2-methylpyridin-3-yl)-2-(1-(2-hydroxyethyl)-1H-pyrazol-4-yl)pyrazolo[5,1-b]thiazole-7-carboxamide, as a off-white solid (28.6 mg). LCMS (ESI): mass calcd. for C₂₄H₂₈N₈O₃S, 508.2; m/z found, 509.3 [M+H]⁺; ¹H NMR (400 MHz, DMSO-d6) δ ppm 1.21 (s, 6H), 2.39 (s, 3H), 3.11 (br s, 4H), 3.26 (br s, 2H), 3.74 (q, J=1.00 Hz, 2H), 4.15 (t, J=1.00 Hz, 2H), 4.95 (t, J=1.00 Hz, 1H), 7.89 (s, 1H), 8.13 (d, J=1.00 Hz, 1H), 8.20 (s, 1H), 8.48 (s, 1H), 8.55 (d, J=1.00 Hz, 1H), 8.59 (s, 1H), 9.84 (s, 1H), 9.91 (br s, 1H).

### Example 292: N-(5-(2-(3,3-dimethylazetidin-1-yl)acetamido)-2-methylpyridin-3-yl)-2-(5-(hydroxymethyl)furan-2-yl)pyrazolo[5,1-b]thiazole-7-carboxamide

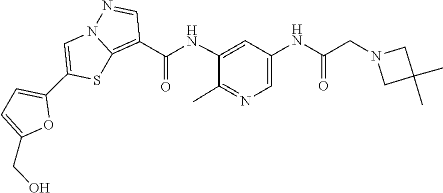 [SMILES: Cc1ncc(NC(=O)CN2CC(C)(C)C2)cc1NC(=O)c1cnn2cc(-c3ccc(CO)o3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), (5-(hydroxymethyl)furan-2-yl)boronic acid (35.67 mg, 0.25 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction was thoroughly flushed with argon before being capped and heated at 100° C. for 17.5 h. The reaction was diluted with MeOH (25 mL) and silica gel (5 g) was added. All solvents were removed in vacuo. The silica gel mesh was loaded on a Redi Sep Rf silica gel cartridge (24 g) eluting with MeOH and CH₂Cl₂ (0-30%) over 15 min to afford a dark brown solid. The solid was dissolved in DMSO (1.5 mL) and was further purified by HPLC (H₂O/ACN, 0.16% NH₄OH, XBridge 19×100) to afford the product, N-(5-(2-(3,3-dimethylazetidin-1-yl)acetamido)-2-methylpyridin-3-yl)-2-(5-(hydroxymethyl)furan-2-yl)pyrazolo[5,1-b]thiazole-7-carboxamide, as an off-white solid (15.4 mg). LCMS (ESI): mass calcd. for C₂₄H₂₆N₆O₄S, 494.2; m/z found, 495.2 [M+H]⁺; ¹H NMR (400 MHz, DMSO-d6) δ ppm 1.20 (s, 6H), 2.40 (s, 3H), 3.16 (d, J=1.00 Hz, 4H), 3.39 (br s, 2H), 4.44 (d, J=1.00 Hz, 2H), 5.35 (t, J=1.00 Hz, 1H), 6.48 (d, J=1.00 Hz, 1H), 6.92 (d, J=1.00 Hz, 1H), 8.13 (d, J=1.00 Hz, 1H), 8.53-8.59 (m, 2H), 8.72 (s, 1H), 9.89 (s, 1H), 9.93-10.03 (m, 1H).

### Example 293: N-(5-(2-(3,3-dimethylazetidin-1-yl)acetamido)-2-methylpyridin-3-yl)-2-(1-(piperidin-4-yl)-1H-pyrazol-4-yl)pyrazolo[5,1-b]thiazole-7-carboxamide

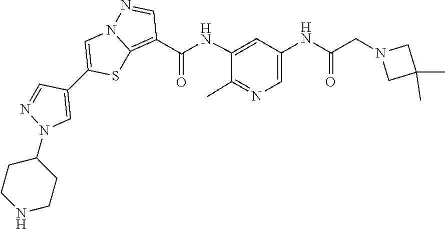 [SMILES: Cc1ncc(NC(=O)CN2CC(C)(C)C2)cc1NC(=O)c1cnn2cc(-c3cnn(C4CCNCC4)c3)sc12]

To a mixture of 2-bromo-N-(5-(2-(3,3-dimethylazetidin-1-yl)acetamido)-2-methylpyridin-3-yl)pyrazolo[5,1-b]thiazole-7-carboxamide (100 mg, 0.21 mmol), 4-(4-(4,4,5,5-tetramethyl-1,3,2-dioxaborolan-2-yl)-1H-pyrazol-1-yl)piperidine (116.12 mg, 0.42 mmol), and K₂CO₃ (86.85 mg, 0.63 mmol) in 1,4-dioxane (2.5 mL) and water (0.5 mL) was added 1,1′-bis(diphenylphosphino)ferrocene-palladium(II)dichloride dichloromethane complex (34.21 mg, 0.042 mmol). The reaction was thoroughly flushed with argon before being capped and heated at 100° C. for 17.5 h. The reaction was diluted with MeOH (25 mL) and silica gel (5 g) was added to the reaction. All solvents were removed in vacuo. The silica gel mesh was loaded on a Redi Sep Rf silica gel cartridge (24 g) eluting with MeOH/CH₂Cl₂ (0-30%) over 15 min to afford a grey solid. The solid was taken up in DMSO (2 mL) and was further purified by HPLC (H₂O/ACN, 0.16% NH₄OH, XBridge 19×100) to give the product, N-(5-(2-(3,3-dimethylazetidin-1-yl)acetamido)-2-methylpyridin-3-yl)-2-(1-(piperidin-4-yl)-1H-pyrazol-4-yl)pyrazolo[5,1-b]thiazole-7-carboxamide, as an off-white solid (19.1 mg). LCMS (ESI): mass calcd. for C₂₇H₃₃N₉O₂S, 547.2; m/z found, 548.3 [M+H]⁺; ¹H NMR (400 MHz, DMSO-d6) δ ppm 1.25 (s, 3H), 1.37 (s, 3H), 2.06-2.32 (m, 4H), 2.56 (s, 3H), 3.07 (q, J=1.00 Hz, 2H), 3.41 (br s, 1H), 3.94 (d, J=1.00 Hz, 4H), 4.33 (d, J=1.00 Hz, 2H), 4.46-4.63 (m, 1H), 7.97 (s, 1H), 8.31 (s, 1H), 8.35 (s, 1H), 8.63 (s, 1H), 8.66 (s, 1H), 8.68 (s, 1H), 8.82-9.01 (m, 1H), 9.04-9.20 (m, 1H), 10.92 (br s, 1H), 11.36 (br s, 1H).

### Example 294: 2-(1-cyclopropyl-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide

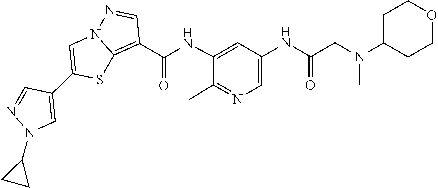 [SMILES: Cc1ncc(NC(=O)CN(C)C2CCOCC2)cc1NC(=O)c1cnn2cc(-c3cnn(C4CC4)c3)sc12]

### Step a: 2-bromo-N-(2-methyl-5-(2-(methyl(tetrahydro-2H-pyran-4-yl)amino)acetamido)pyridin-3-yl)pyrazolo[5,1-b]thiazole-7-carboxamide

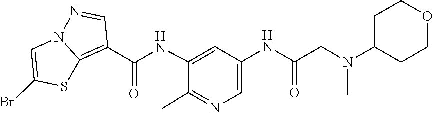 [SMILES: Cc1ncc(NC(=O)CN(C)C2CCOCC2)cc1NC(=O)c1cnn2cc(Br)sc12]

A mixture of 2-bromo-N-(5-(2-chloroacetamido)-2-methylpyridin-3-yl)pyrazolo[5,1-b]thiazole-7-carboxamide (1.5 g, 3.5 mmol), N-methyltetrahydro-2H-pyran-4-amine (0.48 g, 4.20 mmol), K₂CO₃ (1.45 g, 10.50 mmol), and NaI (190 mg) in DMF (25 mL) was heated at 50° C. For 20.5 h. The reaction was poured into water (250 mL) with stirring. The precipitate was collected via filtration, air dried, then continued to be dried under high vacuum to afford the product, 2-bromo-N-(2-methyl-5-(2-(methyl(tetrahydro-2H-pyran-4-yl)amino)acetamido)pyridin-3-yl)pyrazolo[5,1-b]thiazole-7-carboxamide, as a tan solid (1.5021 g). The product was used without further purification. LCMS (ESI): mass calcd. for C₂₀H₂₃BrN₆O₃S, 506.1/508.1; m/z found, 507.1/509.1 [M+H]⁺; ¹H NMR (400 MHz, DMSO-d6) δ ppm 1.45 (dq, J=1.00 Hz, 2H), 1.74 (d, J=1.00 Hz, 2H), 2.31 (s, 3H), 2.40 (s, 3H), 2.66 (tt, J=1.00 Hz, 1H), 3.22 (s, 2H), 3.27 (t, J=1.00 Hz, 2H), 3.89 (dd, J=1.00 Hz, 2H), 8.19 (d, J=1.00 Hz, 1H), 8.56-8.65 (m, 2H), 8.79 (s, 1H), 9.86 (s, 1H), 9.96 (s, 1H).

### Step b: 2-(1-cyclopropyl-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide

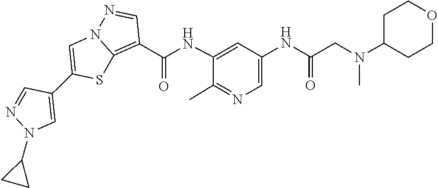 [SMILES: Cc1ncc(NC(=O)CN(C)C2CCOCC2)cc1NC(=O)c1cnn2cc(-c3cnn(C4CC4)c3)sc12]

To a mixture of 2-bromo-N-(2-methyl-5-(2-(methyl(tetrahydro-2H-pyran-4-yl)amino)acetamido)pyridin-3-yl)pyrazolo[5,1-b]thiazole-7-carboxamide (100 mg, 0.20 mmol), 1-cyclopropyl-4-(4,4,5,5-tetramethyl-1,3,2-dioxaborolan-2-yl)-1H-pyrazole (55.37 mg, 0.24 mmol), and Cs₂CO₃ (192.64 mg, 0.59 mmol) in 1,4-dioxane (2.5 mL) and water (0.5 mL) was added DPPF Pd G4 (18.49 mg, 0.020 mmol). The reaction mixture was thoroughly flushed with argon before being capped and heated at 90° C. For 22.5 h. LCMS indicated a mixture of the starting bromide and product (˜1:1). The reaction mixture was diluted with MeOH (25 mL) and silica gel (3 g) was added. All solvents were removed in vacuo. The silica gel mesh was loaded on a Redi Sep Rf silica gel cartridge (24 g) eluthing with MeOH/CH₂Cl₂ (0-30%) over 15 min to afford the product, 2-(1-cyclopropyl-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide, as a brown solid (27.9 mg). LCMS (ESI): mass calcd. for C₂₆H₃₀N₈O₃S, 534.2; m/z found, 535.3 [M+H]⁺; ¹H NMR (400 MHz, DMSO-d6) δ ppm 0.95-1.05 (m, 2H), 1.05-1.13 (m, 2H), 1.46 (dq, J=1.00 Hz, 2H), 1.67-1.79 (m, 2H), 2.31 (s, 3H), 2.40 (s, 3H), 2.66 (tt, J=1.00 Hz, 1H), 3.22 (s, 2H), 3.27 (s, 2H), 3.77 (spt, J=1.00 Hz, 1H), 3.89 (dd, J=1.00 Hz, 2H), 7.87 (s, 1H), 8.18 (d, J=1.00 Hz, 1H), 8.30 (s, 1H), 8.51 (s, 1H), 8.56 (s, 1H), 8.60 (d, J=1.00 Hz, 1H), 9.86 (s, 1H), 9.89 (s, 1H).

### Example 295: 2-(1-(difluoromethyl)-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide

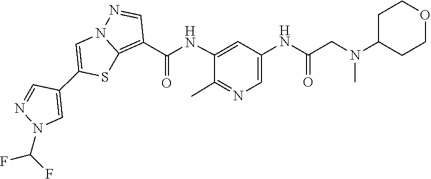 [SMILES: Cc1ncc(NC(=O)CN(C)C2CCOCC2)cc1NC(=O)c1cnn2cc(-c3cnn(C(F)F)c3)sc12]

To a mixture of 2-bromo-N-(2-methyl-5-(2-(methyl(tetrahydro-2H-pyran-4-yl)amino)acetamido)pyridin-3-yl)pyrazolo[5,1-b]thiazole-7-carboxamide (100 mg, 0.20 mmol), 1-(difluoromethyl)-4-(4,4,5,5-tetramethyl-1,3,2-dioxaborolan-2-yl)-1H-pyrazole (57.72 mg, 0.24 mmol), and K₂CO₃ (81.71 mg, 0.59 mmol) in 1,4-dioxane (2.5 mL) and water (0.5 mL) was added 1,1′-bis(diphenylphosphino)ferrocene-palladium(II)dichloride dichloromethane complex (32.19 mg, 0.039 mmol). The reaction was thoroughly flushed with argon before being capped and heated at 100° C. for 27.5 h. The reaction was diluted with MeOH (10 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min to afford the product, 2-(1-(difluoromethyl)-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide, as a brown solid (40.7 mg). LCMS (ESI): mass calcd. for C₂₄H₂₆F₂N₈O₃S, 544.2; m/z found, 545.3 [M+H]⁺; ¹H NMR (400 MHz, DMSO-d6) δ ppm 1.45 (dq, J=1.00 Hz, 2H), 1.73 (d, J=1.00 Hz, 2H), 2.31 (s, 3H), 2.40 (s, 3H), 2.66 (tt, J=1.00 Hz, 1H), 3.15 (d, J=1.00 Hz, 1H), 3.22 (s, 2H), 3.27 (t, J=1.00 Hz, 2H), 3.89 (dd, J=1.00 Hz, 2H), 7.87 (t, J=1.00 Hz, 1H), 8.18 (d, J=1.00 Hz, 1H), 8.30 (s, 1H), 8.54 (s, 1H), 8.58-8.63 (m, 1H), 8.79 (d, J=1.00 Hz, 1H), 9.84 (d, J=1.00 Hz, 0.5H), 9.90 (d, J=1.00 Hz, 1.5H); ¹⁹F NMR (376 MHz, DMSO-d6) δ ppm −94.52 (s, 2F)

### Example 296: 2-(1-cyclobutyl-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide

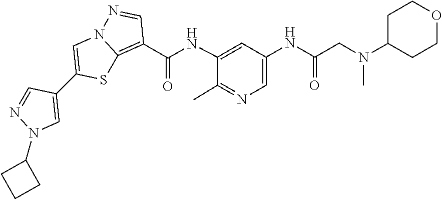 [SMILES: Cc1ncc(NC(=O)CN(C)C2CCOCC2)cc1NC(=O)c1cnn2cc(-c3cnn(C4CCC4)c3)sc12]

To a mixture of 2-bromo-N-(2-methyl-5-(2-(methyl(tetrahydro-2H-pyran-4-yl)amino)acetamido)pyridin-3-yl)pyrazolo[5,1-b]thiazole-7-carboxamide (100 mg, 0.20 mmol), 1-cyclobutyl-4-(4,4,5,5-tetramethyl-1,3,2-dioxaborolan-2-yl)-1H-pyrazole (58.68 mg, 0.24 mmol), and Cs₂CO₃ (192.64 mg, 0.59 mmol) in 1,4-dioxane (2.5 mL) and water (0.5 mLl) was added DPPF Pd G4 (55.47 mg, 0.059 mmol). The reaction mixture was thoroughly flushed with argon before being capped and heated at 90° C. for 48 h. All solvents were removed in vacuo. The reaction was taken up in MeOH (5 mL) and filtered via a syringe filter and the product was purified by HPLC (acetonitrile/water/TFA) to afford the TFA salt of the product, 2-(1-cyclobutyl-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide, as a tan solid (42.0 mg). LCMS (ESI): mass calcd. for C₂₇H₃₂N₈O₃S, 548.2; m/z found, 549.3 [M+H]⁺; ¹H NMR (400 MHz, DMSO-d6) δ ppm 1.59-1.75 (m, 2H), 1.75-1.87 (m, 2H), 1.87-2.07 (m, 2H), 2.33-2.45 (m, 3H), 2.47 (s, 3H), 2.86 (s, 3H), 3.32 (t, J=1.00 Hz, 2H), 3.59 (t, J=1.00 Hz, 1H), 3.98 (dd, J=1.00 Hz, 2H), 4.06 (d, J=1.00 Hz, 1H), 4.33 (d, J=1.00 Hz, 1H), 4.87 (quin, J=1.00 Hz, 1H), 7.93 (s, 1H), 8.19 (d, J=1.00 Hz, 1H), 8.33 (s, 1H), 8.53 (s, 1H), 8.59 (s, 2H), 9.80 (br s, 1H), 9.93 (s, 1H), 10.88 (s, 1H); ¹⁹F NMR (376 MHz, DMSO-d6) δ ppm −76.17-−72.60 (m, 3F).

### Example 297: 2-(1-(1-cyanoethyl)-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide

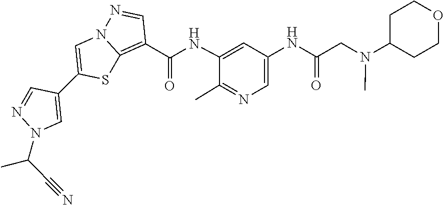 [SMILES: Cc1ncc(NC(=O)CN(C)C2CCOCC2)cc1NC(=O)c1cnn2cc(-c3cnn(C(C)C#N)c3)sc12]

To a mixture of 2-bromo-N-(2-methyl-5-(2-(methyl(tetrahydro-2H-pyran-4-yl)amino)acetamido)pyridin-3-yl)pyrazolo[5,1-b]thiazole-7-carboxamide (100 mg, 0.20 mmol), 2-(4-(4,4,5,5-tetramethyl-1,3,2-dioxaborolan-2-yl)-1H-pyrazol-1-yl)propanenitrile (53.57 mg, 0.22 mmol), and K₂CO₃ (81.71 mg, 0.59 mmol) in 1,4-dioxane (2.5 mL) and water (0.5 mL) was added 1,1′-bis(diphenylphosphino)ferrocene-palladium(II)dichloride dichloromethane complex (32.19 mg, 0.039 mmol). The reaction was thoroughly flushed with argon before being capped and heated at 100° C. for 32 h. The reaction was diluted with MeOH (10 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min to afford the product, 2-(1-(1-cyanoethyl)-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide, as a brown solid (30.0 mg). LCMS (ESI): mass calcd. for C₂₆H₂₉N₉O₃S, 547.2; m/z found, 548.3 [M+H]*; ¹H NMR (400 MHz, DMSO-d6) δ ppm 1.45 (dq, J=1.00 Hz, 2H), 1.69-1.78 (m, 2H), 1.84 (d, J=1.00 Hz, 3H), 2.32 (s, 3H), 2.40 (s, 3H), 2.66 (tt, J=1.00 Hz, 1H), 3.17 (d, J=1.00 Hz, 2H), 3.22 (s, 2H), 3.24-3.31 (m, 2H), 5.90 (q, J=1.00 Hz, 1H), 8.10 (s, 1H), 8.16-8.21 (m, 1H), 8.44 (s, 1H), 8.53 (s, 1H), 8.58-8.63 (m, 1H), 8.68 (s, 1H), 9.84 (d, J=1.00 Hz, 0.5H), 9.89 (d, J=1.00 Hz, 1.5H).

### Example 298: N-(2-methyl-5-(2-(methyl(tetrahydro-2H-pyran-4-yl)amino)acetamido)pyridin-3-yl)-2-(1-((methylsulfonyl)methyl)-1H-pyrazol-4-yl)pyrazolo[5,1-b]thiazole-7-carboxamide

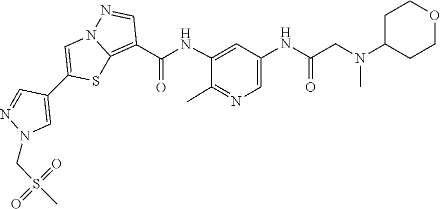 [SMILES: Cc1ncc(NC(=O)CN(C)C2CCOCC2)cc1NC(=O)c1cnn2cc(-c3cnn(CS(C)(=O)=O)c3)sc12]

To a mixture of 2-bromo-N-(2-methyl-5-(2-(methyl(tetrahydro-2H-pyran-4-yl)amino)acetamido)pyridin-3-yl)pyrazolo[5,1-b]thiazole-7-carboxamide (100 mg, 0.20 mmol), 1-((methylsulfonyl)methyl)-4-(4,4,5,5-tetramethyl-1,3,2-dioxaborolan-2-yl)-1H-pyrazole (67.68 mg, 0.24 mmol), and K₂CO₃ (81.71 mg, 0.59 mmol) in 1,4-dioxane (2.5 mL) and water (0.5 mLl) was added 1,1′-bis(diphenylphosphino)ferrocene-palladium(II)dichloride dichloromethane complex (32.19 mg, 0.039 mmol). The reaction was thoroughly flushed with argon before being capped and heated at 100° C. for 20.5 h. The reaction was diluted with MeOH (25 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min to afford the product, N-(2-methyl-5-(2-(methyl(tetrahydro-2H-pyran-4-yl)amino)acetamido)pyridin-3-yl)-2-(1-((methylsulfonyl)methyl)-1H-pyrazol-4-yl)pyrazolo[5,1-b]thiazole-7-carboxamide, as a brown solid (40.1 mg). LCMS (ESI): mass calcd. for C₂₅H₃₀N₈O₅S₂, 586.2; m/z found, 587.3 [M+H]⁺; ¹H NMR (400 MHz, DMSO-d6) δ ppm 1.45 (dq, J=1.00 Hz, 2H), 1.73 (d, J=1.00 Hz, 2H), 2.32 (s, 3H), 2.41 (s, 3H), 2.66 (tt, J=1.00 Hz, 1H), 3.07 (s, 3H), 3.22 (s, 2H), 3.27 (t, J=1.00 Hz, 2H), 3.90 (dd, J=1.00 Hz, 2H), 5.80 (s, 2H), 8.12 (s, 1H), 8.19 (d, J=1.00 Hz, 1H), 8.37 (s, 1H), 8.53 (s, 1H), 8.61 (d, J=1.00 Hz, 1H), 8.73 (s, 1H), 9.86 (s, 1H), 9.91 (s, 1H).

### Examples 299: 2-(1-(cyanomethyl)-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide and Example 300: 2-(1-(2-amino-2-oxoethyl)-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide

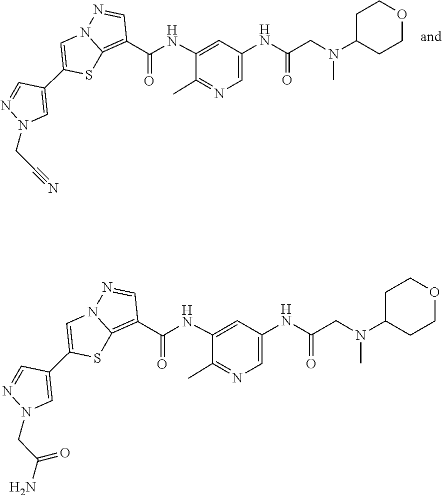 [SMILES: Cc1ncc(NC(=O)CN(C)C2CCOCC2)cc1NC(=O)c1cnn2cc(-c3cnn(CC#N)c3)sc12 ; Cc1ncc(NC(=O)CN(C)C2CCOCC2)cc1NC(=O)c1cnn2cc(-c3cnn(CC(N)=O)c3)sc12]

To a mixture of 2-bromo-N-(2-methyl-5-(2-(methyl(tetrahydro-2H-pyran-4-yl)amino)acetamido)pyridin-3-yl)pyrazolo[5,1-b]thiazole-7-carboxamide (100 mg, 0.20 mmol), 2-(4-(4,4,5,5-tetramethyl-1,3,2-dioxaborolan-2-yl)-1H-pyrazol-1-yl)acetonitrile (55.122 mg, 0.24 mmol), and K₂CO₃ (81.71 mg, 0.59 mmol) in 1,4-dioxane (2.5 mL) and water (0.5 mL) was added 1,1′-bis(diphenylphosphino)ferrocene-palladium(II)dichloride dichlormethane complex (32.19 mg, 0.039 mmol). The reaction was thoroughly flushed with argon before being capped and heated at 100° C. for 24 h. The reaction was diluted with MeOH (10 mL) and silica gel (3 g) was added. All solvents were removed in vacuo. The silica gel mesh was loaded on a Redi Sep Rf silica gel cartridge (24 g) eluting with MeOH/CH₂Cl₂ (0-30%) over 15 min then 30% MeOH over 3 min to afford two products, 2-(1-(cyanomethyl)-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide [brown solid, 13.4 mg; LCMS (ESI): mass calcd. for C₂₅H₂₇N₉O₃S, 533.2; m/z found, 534.3 [M+H]⁺; ¹H NMR (400 MHz, DMSO-d6) δ ppm 1.46 (dq, J=1.00 Hz, 2H), 1.74 (d, J=1.00 Hz, 2H), 2.32 (s, 3 H), 2.41 (s, 3H), 2.58-2.73 (m, 1H), 3.21 (s, 2H), 3.27 (t, J=1.00 Hz, 2H), 3.89 (dd, J=1.00 Hz, 2H), 5.56 (s, 2H), 8.07 (s, 1H), 8.19 (d, J=1.00 Hz, 1H), 8.34 (s, 1H), 8.52 (s, 1H), 8.60 (d, J=1.00 Hz, 1H), 8.68 (s, 1H), 9.86 (s, 1H), 9.91 (s, 1H)], and 2-(1-(2-amino-2-oxoethyl)-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide [brown solid (26.5 mg); LCMS (ESI): mass calcd. for C₂₅H₂₉N₉O₄S, 551.2; m/z found, 552.3 [M+H]⁺; ¹H NMR (400 MHz, DMSO-d6) δ ppm 1.45 (dq, J=1.00 Hz, 2H) 1.74 (d, J=1.00 Hz, 2H), 2.32 (s, 3H), 2.41 (s, 3H), 2.59-2.74 (m, 1H), 3.22 (s, 2H), 3.28 (t, J=1.00 Hz, 2H), 3.90 (dd, J=1.00 Hz, 2H), 4.81 (s, 2H), 7.30 (s, 1H), 7.59 (s, 1H), 7.91 (s, 1H), 8.17 (d, J=1.00 Hz, 1H), 8.21 (s, 1H), 8.53 (s, 1H), 8.62 (s, 2H), 9.85 (s, 1H), 9.91 (s, 1H)], respectively.

### Example 301. (S)-2-(2-methoxypyridin-3-yl)-N-(2-methyl-5-((2-(2-methylpyrrolidin-1-yl)ethyl)carbamoyl)pyridin-3-yl)pyrazolo[5,1-b]thiazole-7-carboxamide

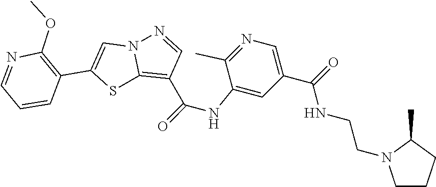 [SMILES: COc1ncccc1-c1cn2ncc(C(=O)Nc3cc(C(=O)NCCN4CCC[C@@H]4C)cnc3C)c2s1]

### Step a: (S)-2-bromo-N-(2-methyl-5-((2-(2-methylpyrrolidin-1-yl)ethyl)carbamoyl)pyridin-3-yl)pyrazolo[5,1-b]thiazole-7-carboxamide

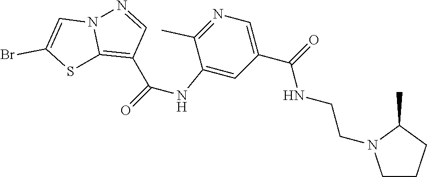 [SMILES: Cc1ncc(C(=O)NCCN2CCC[C@@H]2C)cc1NC(=O)c1cnn2cc(Br)sc12]

A 30 mL vial, equipped with a stir bar, was charged with (S)-2-methyl-pyrrolidine (1.13 g, 12.8 mmol), DMF (10 mL), K₂CO₃ (3.56 g, 25.8 mmol), and tert-butyl 2-bromoethylcarbamate (3.22 g, 14.1 mmol). A nitrogen atmosphere was established. The reaction proceeded at 23° C. After 8 days, the reaction was quenched with water (80 mL). Extract was obtained using EtOAc (3×20 mL). The organic phase was concentrated under reduced pressure to form tert-butyl (S)-(2-(2-methylpyrrolidin-1-yl)ethyl)carbamate. A 30 mL vial, containing tert-butyl (S)-(2-(2-methylpyrrolidin-1-yl)ethyl)carbamate, was charged with a stir bar, hydrogen chloride solution in 4.0M in dioxane (20.0 mL, 80.2 mmol). A nitrogen atmosphere was established. The reaction proceeded at 23° C. overnight. Solvent was removed to form the HCl salt of (S)-2-(2-methylpyrrolidin-1-yl)ethan-1-amine.

A 250 mL round bottom flask, containing (S)-2-(2-methylpyrrolidin-1-yl)ethan-1-amine (1.64 g, 12.8 mmol), was charged with a stir bar, DMF (30 mL) and N,N-diisopropylethylamine (6.0 mL, 34 mmol). The mixture was stirred for 5 min. 5-(2-Bromopyrazolo[5,1-b]thiazole-7-carboxamido)-6-methylnicotinic acid (2.36 g, 6.18 mmol) in DMF (20 mL) was added to the reaction pot. HATU (3.14 g, 8.25 mmol) was added. The reaction was placed under a nitrogen atmosphere. The reaction proceeded at 21° C. After approximately 6 d 18 h, solvent was removed under reduced pressure at 50° C. The crude residue was allowed to cool to room temperature. The residue was purified by preparative high-performance liquid chromatography over column: Waters XBridge BEH C18 5 μm to provide (S)-2-bromo-N-(2-methyl-5-((2-(2-methylpyrrolidin-1-yl)ethyl)carbamoyl)pyridin-3-yl)pyrazolo[5,1-b]thiazole-7-carboxamide as a white solid (528 mg, 17%). LCMS (ESI): mass calcd for C₂₀H₂₃BrN₆O₂S, 490.1; m/z found, 491.1 [M+H]⁺.

### Step b: (S)-2-(2-methoxypyridin-3-yl)-N-(2-methyl-5-((2-(2-methylpyrrolidin-1-yl)ethyl)carbamoyl)pyridin-3-yl)pyrazolo[5,1-b]thiazole-7-carboxamide

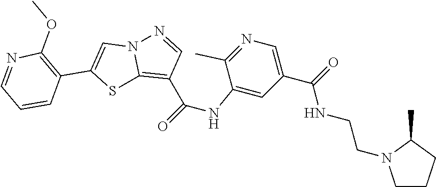 [SMILES: COc1ncccc1-c1cn2ncc(C(=O)Nc3cc(C(=O)NCCN4CCC[C@@H]4C)cnc3C)c2s1]

(S)-2-bromo-N-(2-methyl-5-((2-(2-methylpyrrolidin-1-yl)ethyl)carbamoyl)pyridin-3-yl)pyrazolo[5,1-b]thiazole-7-carboxamide (124 mg, 0.253 mmol), 2-methoxypyridine-3-boronic acid pinacol ester (161 mg, 0.672 mmol), cesium carbonate (333 mg, 1.01 mmol), 1,1′-bis(diphenylphosphino)ferrocene-palladium(II)dichloride dichloromethane complex (36.2 mg, 0.0443 mmol), and 1,4-dioxane:distilled water (5:1) (3 mL) were combined in a 2-5 mL microwave vial, equipped with a stir bar. The vial was sealed with a cap. The contents were sparged with nitrogen with vigorous stirring. The mixture was irradiated in a Biotage Initiator+microwave reactor at 130° C. for 1 h and allowed to cool to room temperature. Solvent was removed under reduced pressure in a 20 mL vial. The crude residue was dissolved in MeOH. Si-Trisamine was added. The mixture was stirred at room temperature for approximately 15.5 h. The crude mixture was filtered using a Biotage phase separator. Solvent was removed under reduced pressure. The residue was purified by preparative high-performance liquid chromatography over column: Waters XBridge BEH C18 5 μm to give (S)-2-(2-methoxypyridin-3-yl)-N-(2-methyl-5-((2-(2-methylpyrrolidin-1-yl)ethyl)carbamoyl)pyridin-3-yl)pyrazolo[5,1-b]thiazole-7-carboxamide as a white solid (17 mg, 12%). LCMS (ESI): mass calcd for C₂₆H₂₉N₇O₃S, 519.2; m/z found, 520.1 [M+H]+. ¹H NMR (CHLOROFORM-d, 400 MHz) δ 8.73 (d, 1H, J=2.2 Hz), 8.64 (d, 1H, J=2.2 Hz), 8.48 (s, 1H), 8.20 (dd, 1H, J=2.0, 4.9 Hz), 8.16 (s, 1H), 7.80 (dd, 1H, J=1.7, 7.6 Hz), 7.59 (br s, 1H), 7.13 (br s, 1H), 7.03 (dd, 1H, J=4.9, 7.6 Hz), 4.14 (s, 3H), 3.7-3.8 (m, 1H), 3.3-3.4 (m, 1H), 3.2-3.2 (m, 1H), 3.0-3.1 (m, 1H), 2.65 (s, 3H), 2.4-2.5 (m, 2H), 2.22 (q, 1H, J=8.6 Hz), 1.9-2.0 (m, 1H), 1.7-1.9 (m, 2H), 1.4-1.5 (m, 1H), 1.13 (d, 3H, J=6.1 Hz).

### Example 302: 2-(1-methyl-1H-pyrazol-4-yl)-N-(2-methyl-5-(2-(tetrahydro-1H-pyrrolizin-7a(5H)-yl)acetamido)pyridin-3-yl)pyrazolo[5,1-b]thiazole-7-carboxamide

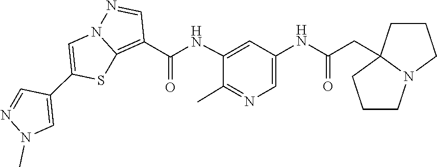 [SMILES: Cc1ncc(NC(=O)CC23CCCN2CCC3)cc1NC(=O)c1cnn2cc(-c3cnn(C)c3)sc12]

To a solution of N-(5-amino-2-methylpyridin-3-yl)-2-(1-methyl-1H-pyrazol-4-yl)pyrazolo[5,1-b]thiazole-7-carboxamide hydrochloride (60 mg, 0.15 mmol) and tetrahydro-1H-pyrrolizine-7a(5H)-acetic acid, HCl (37.99 mg, 0.19 mmol) in pyridine (2 mL) was added EDC (41.30 mg, 0.22 mmol). The reaction was stirred at 25° C. for 19.5 h. All solvents were removed in vacuo. The residue was taken up in DMSO (2 mL) and was purified by HPLC (H₂O/ACN, 0.16% NH₄OH, XBridge 19×100) to afford the product, 2-(1-methyl-1H-pyrazol-4-yl)-N-(2-methyl-5-(2-(tetrahydro-1H-pyrrolizin-7a(5H)-yl)acetamido)pyridin-3-yl)pyrazolo[5,1-b]thiazole-7-carboxamide, as an off-white solid (55.2 mg). LCMS (ESI): mass calcd. for C₂₅H₂₈N₈O₂S, 504.2; m/z found, 505.1 [M+H]⁺; ¹H NMR (400 MHz, DMSO-d6) δ ppm 1.54-1.64 (m, 2H), 1.65-1.73 (m, 2H), 1.73-1.84 (m, 2H), 1.84-1.93 (m, 2H), 2.36 (s, 2H), 2.40 (s, 3H), 2.53-2.63 (m, 2H), 2.88-3.04 (m, 2H), 3.86 (s, 3H), 7.88 (s, 1H), 8.12 (d, J=1.00 Hz, 1H), 8.19 (s, 1H), 8.47 (d, J=1.00 Hz, 1H), 8.50 (s, 1H), 8.57 (s, 1H), 9.86 (s, 1H), 10.51 (s, 1H).

### Example 303: N-(5-(2-(3-hydroxypiperidin-1-yl)acetamido)-2-methylpyridin-3-yl)-2-(1-methyl-1H-pyrazol-4-yl)pyrazolo[5,1-b]thiazole-7-carboxamide

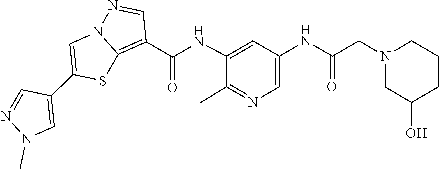 [SMILES: Cc1ncc(NC(=O)CN2CCCC(O)C2)cc1NC(=O)c1cnn2cc(-c3cnn(C)c3)sc12]

To a mixture of N-(5-amino-2-methylpyridin-3-yl)-2-(1-methyl-1H-pyrazol-4-yl)pyrazolo[5,1-b]thiazole-7-carboxamide hydrochloride (60 mg, 0.15 mmol) and 2-(3-hydroxypiperidin-1-yl)acetic acid (29.40 mg, 0.19 mmol) in pyridine (2 mL) was added EDC (41.30 mg, 0.22 mmol). The reaction was stirred at 25° C. for 18 h. All solvents were removed in vacuo. The residue was taken up in DMSO (2 mL) and was purified by HPLC (H₂O/ACN, 0.16% NH₄OH, XBridge 19×100) to afford the product, N-(5-(2-(3-hydroxypiperidin-1-yl)acetamido)-2-methylpyridin-3-yl)-2-(1-methyl-1H-pyrazol-4-yl)pyrazolo[5,1-b]thiazole-7-carboxamide, as an off-white solid (41.9 mg). LCMS (ESI): mass calcd. for C₂₃H₂₆N₈O₃S, 494.2; m/z found, 495.3 [M+H]⁺; ¹H NMR (400 MHz, DMSO-d6) δ ppm 1.10-1.27 (m, 1H), 1.43-1.59 (m, 1H), 1.63-1.79 (m, 2H), 2.07 (t, J=1.00 Hz, 1H), 2.20 (t, J=1.00 Hz, 1H), 2.40 (s, 3H), 2.54-2.65 (m, 1H), 2.75 (dd, J=1.00 Hz, 1H), 3.12 (d, J=1.00 Hz, 2H), 3.54-3.69 (m, 1H), 3.88 (s, 3H), 4.72 (d, J=1.00 Hz, 1H), 7.88 (s, 1H), 8.14 (s, 1H), 8.20 (s, 1H), 8.51 (s, 1H), 8.57 (s, 1H), 8.59 (d, J=1.00 Hz, 1H), 9.88 (s, 1H), 9.91 (s, 1H).

### Example 304: N-(5-((2S,4R)-1,4-dimethylpyrrolidine-2-carboxamido)-2-methylpyridin-3-yl)-2-(1-methyl-1H-pyrazol-4-yl)pyrazolo[5,1-b]thiazole-7-carboxamide

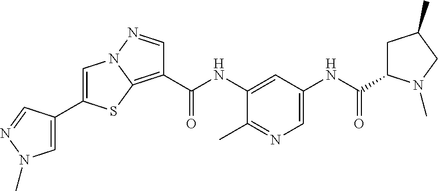 [SMILES: Cc1ncc(NC(=O)[C@@H]2C[C@@H](C)CN2C)cc1NC(=O)c1cnn2cc(-c3cnn(C)c3)sc12]

To a mixture of N-(5-amino-2-methylpyridin-3-yl)-2-(1-methyl-1H-pyrazol-4-yl)pyrazolo[5,1-b]thiazole-7-carboxamide hydrochloride (60 mg, 0.15 mmol) and (2S,4R)-1,4-dimethylpyrrolidine-2-carboxylic acid (26.44 mg, 0.19 mmol) in pyridine (2 mL) was added EDCI (41.30 mg, 0.22 mmol). The reaction was stirred at 25° C. for 18.5 h. All solvents were removed in vacuo. The residue was taken up in DMSO (2 mL) and was purified by HPLC (H₂O/ACN, 0.16% NH₄OH, XBridge 19×100) to afford the product, N-(5-((2S,4R)-1,4-dimethylpyrrolidine-2-carboxamido)-2-methylpyridin-3-yl)-2-(1-methyl-1H-pyrazol-4-yl)pyrazolo[5,1-b]thiazole-7-carboxamide, as an off-white solid (31.1 mg). LCMS (ESI): mass calcd. for C₂₃H₂₆N₈O₂S, 478.2; m/z found, 479.3 [M+H]⁺; ¹H NMR (400 MHz, DMSO-d6) δ ppm 0.98 (d, J=1.00 Hz, 3H), 1.65-1.84 (m, 1H), 1.93-2.07 (m, 2H), 2.19-2.32 (m, 1H), 2.35 (s, 3H), 2.41 (s, 3H), 2.99-3.09 (m, 1H), 3.17-3.25 (m, 1H), 3.88 (s, 3H), 7.87 (s, 1H), 8.19 (s, 1H), 8.22 (d, J=1.00 Hz, 1H), 8.49 (s, 1H), 8.57 (s, 1H), 8.63 (d, J=1.00 Hz, 1H), 9.84 (s, 1H), 9.95 (s, 1H).

### Example 305: (S)—N-(5-(2-(1-isopropylpiperidin-2-yl)acetamido)-2-methylpyridin-3-yl)-2-(1-methyl-1H-pyrazol-4-yl)pyrazolo[5,1-b]thiazole-7-carboxamide

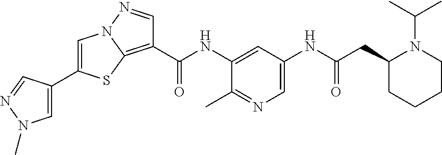 [SMILES: Cc1ncc(NC(=O)C[C@@H]2CCCCN2C(C)C)cc1NC(=O)c1cnn2cc(-c3cnn(C)c3)sc12]

To a solution of (S)-2-(1-isopropylpiperidin-3-yl)acetic acid (50 mg, 0.27 mmol) and N-(5-amino-2-methylpyridin-3-yl)-2-(1-methyl-1H-pyrazol-4-yl)pyrazolo[5,1-b]thiazole-7-carboxamide hydrochloride (105.22 mg, 0.27 mmol) in pyridine (2 mL) was added EDCI (62 mg, 0.32 mmol). The reaction was stirred at 25° C. for 16 h. All solvents were removed in vacuo. The residue was dissolved in DMSO (1.5 mL) and was purified by HPLC (H₂O/ACN, 0.16% NH₄OH, XBridge 19×100) to afford the product, (S)—N-(5-(2-(1-isopropylpiperidin-2-yl)acetamido)-2-methylpyridin-3-yl)-2-(1-methyl-1H-pyrazol-4-yl)pyrazolo[5,1-b]thiazole-7-carboxamide, as an off-white solid (33.2 mg). LCMS (ESI): mass calcd. for C₂₆H₃₂N₈O₂S, 520.2; m/z found, 521.3 [M+H]⁺; ¹H NMR (400 MHz, DMSO-d6) δ ppm 0.94 (dd, J=6.36, 3.42 Hz, 6H), 0.95-1.04 (m, 1H), 1.33-1.50 (m, 1H), 1.53-1.74 (m, 2H), 1.84-2.04 (m, 1H), 2.04-2.16 (m, 1H), 2.16-2.34 (m, 2H), 2.35-2.44 (m, 3H), 2.57-2.78 (m, 4H), 3.83-3.93 (m, 3H), 7.88 (s, 1H), 8.11 (d, J=1.00 Hz, 1H), 8.19 (s, 1H), 8.48 (s, 2H), 8.58 (s, 1H), 9.85 (s, 1H), 10.08 (s, 1H).

### Example 306: N-(5-(2-(azepan-1-yl)propanamido)-2-methylpyridin-3-yl)-2-(1-methyl-1H-pyrazol-4-yl)pyrazolo[5,1-b]thiazole-7-carboxamide

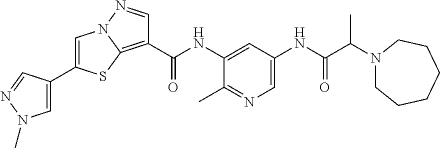 [SMILES: Cc1ncc(NC(=O)C(C)N2CCCCCC2)cc1NC(=O)c1cnn2cc(-c3cnn(C)c3)sc12]

To a mixture of N-(5-amino-2-methylpyridin-3-yl)-2-(1-methyl-1H-pyrazol-4-yl)pyrazolo[5,1-b]thiazole-7-carboxamide hydrochloride (60 mg, 0.15 mmol) and 2-(azepan-1-yl)propanoic acid (31.62 mg, 0.19 mmol) in pyridine (2 mL) was added EDCI (41.30 mg, 0.22 mmol). The reaction was stirred at 25° C. For 20 h. All solvents were removed in vacuo. The residue was taken up in DMSO (2 mL) and was purified by HPLC (H₂O/ACN, 0.16% NH₄OH, XBridge 19×100) to afford the product, N-(5-(2-(azepan-1-yl)propanamido)-2-methylpyridin-3-yl)-2-(1-methyl-1H-pyrazol-4-yl)pyrazolo[5,1-b]thiazole-7-carboxamide, as an off-white solid (39.6 mg). LCMS (ESI): mass calcd. for C₂₅H₃₀N₈O₂S, 506.2; m/z found, 507.3 [M+H]⁺; ¹H NMR (400 MHz, DMSO-d6) δ ppm 1.17 (d, J=1.00 Hz, 3H), 1.49-1.65 (m, 8H), 2.40 (s, 3H), 2.59-2.76 (m, 4H), 3.47 (q, J=1.00 Hz, 1H), 3.88 (s, 3H), 7.88 (s, 1H), 8.16 (d, J=1.00 Hz, 1H), 8.20 (s, 1H), 8.50 (s, 1H), 8.56 (s, 1H), 8.58 (s, 1H), 9.86 (s, 1H), 9.89 (s, 1H).

### Example 307: 2-(1-methyl-1H-pyrazol-4-yl)-N-(2-methyl-5-(3-(piperidin-1-yl)butanamido)pyridin-3-yl)pyrazolo[5,1-b]thiazole-7-carboxamide

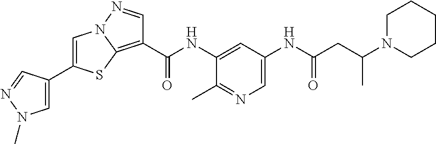 [SMILES: Cc1ncc(NC(=O)CC(C)N2CCCCC2)cc1NC(=O)c1cnn2cc(-c3cnn(C)c3)sc12]

To a mixture of N-(5-amino-2-methylpyridin-3-yl)-2-(1-methyl-1H-pyrazol-4-yl)pyrazolo[5,1-b]thiazole-7-carboxamide hydrochloride (60 mg, 0.15 mmol) and 3-(piperidin-1-yl)butanoic acid (31.62 mg, 0.19 mmol) in pyridine (2 mL) was added EDCI (41.30 mg, 0.22 mmol). The reaction was stirred at 25° C. for 19.5 h. All solvents were removed in vacuo. The residue was taken up in DMSO (2 mL) and was purified by HPLC (H₂O/ACN, 0.16% NH₄OH, XBridge 19×100) to afford the product, 2-(1-methyl-1H-pyrazol-4-yl)-N-(2-methyl-5-(3-(piperidin-1-yl)butanamido)pyridin-3-yl)pyrazolo[5,1-b]thiazole-7-carboxamide, as an off-white solid (48.0 mg). LCMS (ESI): mass calcd. for C₂₅H₃₀N₈O₂S, 506.2; m/z found, 507.3 [M+H]⁺; ¹H NMR (400 MHz, DMSO-d6) δ ppm 1.04 (d, J=1.00 Hz, 3H), 1.29-1.42 (m, 2H), 1.42-1.56 (m, 4H), 2.19 (dd, J=1.00 Hz, 1H), 2.25-2.36 (m, 2H), 2.39 (s, 3H), 2.43-2.47 (m, 2H), 2.53-2.63 (m, 1H), 2.64-2.83 (m, 1H), 3.87 (s, 3H), 7.87 (s, 1H), 8.14 (d, J=1.00 Hz, 1H), 8.20 (s, 1H), 8.48-8.52 (m, 2H), 8.58 (s, 1H), 9.85 (s, 1H), 10.36 (s, 1H).

### Example 308: 2-(1-methyl-1H-pyrazol-4-yl)-N-(2-methyl-5-((1R,9aR)-octahydro-2H-quinolizine-1-carboxamido)pyridin-3-yl)pyrazolo[5,1-b]thiazole-7-carboxamide

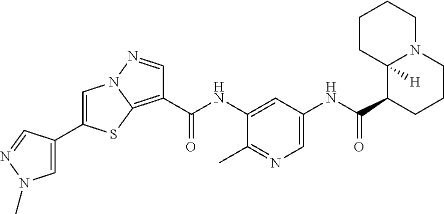

To a mixture of N-(5-amino-2-methylpyridin-3-yl)-2-(1-methyl-1H-pyrazol-4-yl)pyrazolo[5,1-b]thiazole-7-carboxamide hydrochloride (60 mg, 0.15 mmol) and (1R,9aR)-octahydro-2H-quinlizine-1-carboxylic acid hydrochloride (40.58 mg, 0.19 mmol) in pyridine (2 mL) was added EDCI (41.30 mg, 0.22 mmol). The reaction was stirred at 25° C. for 20 h. All solvents were removed in vacuo. The residue was taken up in DMSO (2 mL) and was purified by HPLC (H₂O/ACN, 0.16% NH₄OH, XBridge 19×100) to afford the product, 2-(1-methyl-1H-pyrazol-4-yl)-N-(2-methyl-5-((1R,9aR)-octahydro-2H-quinolizine-1-carboxamido)pyridin-3-yl)pyrazolo[5,1-b]thiazole-7-carboxamide, as an off-white solid (36.2 mg). LCMS (ESI): mass calcd. for C₂₆H₃₀N₈O₂S, 518.2; m/z found, 519.3 [M+H]⁺; ¹H NMR (400 MHz, DMSO-d6) δ ppm 1.17-1.32 (m, 1H), 1.40-1.55 (m, 3H), 1.55-1.67 (m, 3H), 1.67-1.83 (m, 2H), 1.83-1.95 (m, 1H), 1.95-2.17 (m, 3H), 2.39 (s, 3H), 2.42-2.48 (m, 1H), 2.88-3.02 (m, 2H), 3.88 (s, 3H), 7.88 (s, 1H), 8.09 (d, J=1.00 Hz, 1H), 8.19 (s, 1H), 8.47 (d, J=1.00 Hz, 1H), 8.50 (s, 1H), 8.58 (s, 1H), 9.89 (s, 1H), 11.06 (s, 1H).

### Example 309: (R)—N-(5-(2-(1-isopropylpyrrolidin-2-yl)acetamido)-2-methylpyridin-3-yl)-2-(2-methoxypyridin-3-yl)pyrazolo[5,1-b]thiazole-7-carboxamide

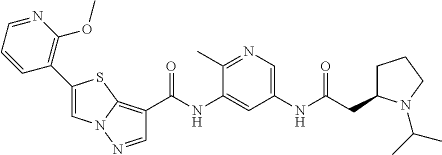 [SMILES: COc1ncccc1-c1cn2ncc(C(=O)Nc3cc(NC(=O)C[C@H]4CCCN4C(C)C)cnc3C)c2s1]

### Step a: tert-butyl (5-(2-(2-methoxypyridin-3-yl)pyrazolo[5,1-b]thiazole-7-carboxamido)-6-methylpyridin-3-yl)carbamate

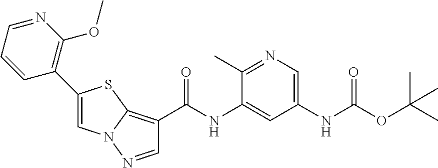 [SMILES: COc1ncccc1-c1cn2ncc(C(=O)Nc3cc(NC(=O)OC(C)(C)C)cnc3C)c2s1]

To a suspension of tert-butyl (5-(6-bromo-[1,2,3]triazolo[1,5-a]pyridine-3-carboxamido)-6-methylpyridin-3-yl)carbamate (3 g, 6.63 mmol) and 2-methoxypyridine-3-boronic acid, pinacol ester (2.34 g, 9.95 mmol) in 1,4-dioxane (30 mL) was added K₂CO₃ (2.75 g, 19.90 mmol) and water (6 mL) followed by the addition of 1,1′-bis(diphenylphosphino)ferrocene-palladium(II)dichloride dichloromethane complex (1.08 g, 1.32 mmol). The reaction was thoroughly flushed with argon before being capped and heated at 100° C. for 18 h. The reaction was removed from the heat and allowed to cool to 25° C. Silica gel (10 g) was added and all solvents were removed in vacuo. The silica gel mesh was loaded on a Redi Sep Rf silica gel cartridge (80 g) eluting with EtOAc over 15 min then MeOH/EtOAc (0-5%) over 5 min to afford the product, tert-butyl (5-(2-(2-methoxypyridin-3-yl)pyrazolo[5,1-b]thiazole-7-carboxamido)-6-methylpyridin-3-yl)carbamate, as a light yellow solid (2.0731 g). LCMS (ESI): mass calcd. for C₂₃H₂₄N604S, 480.2; m/z found, 481.2 [M+H]⁺; ¹H NMR (400 MHz, DMSO-d6) δ ppm 1.50 (s, 9H), 2.36 (s, 3H), 4.08 (s, 3H), 7.34 (s, 1H), 8.01 (s, 1H), 8.17-8.31 (m, 2H), 8.37 (s, 1H), 8.57 (s, 1H), 8.98 (s, 1H), 9.56 (br s, 1H), 9.86 (s, 1H).

### Step b: N-(5-amino-2-methylpyridin-3-yl)-2-(2-methoxypyridin-3-yl)pyrazolo[5,1-b]thiazole-7-carboxamide

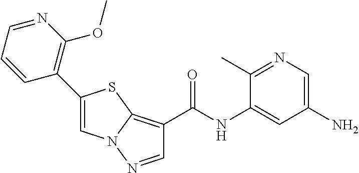 [SMILES: COc1ncccc1-c1cn2ncc(C(=O)Nc3cc(N)cnc3C)c2s1]

A solution of tert-butyl (5-(2-(2-methoxypyridin-3-yl)pyrazolo[5,1-b]thiazole-7-carboxamido)-6-methylpyridin-3-yl)carbamate (2 g, 4.1 mmol) in 25% TFA/CH₂Cl₂ (20 mL) was stirred at 25° C. for 1 h. LCMS indicated the starting BOC had been consumed. Toluene (50 mL) was added and all solvents were removed in vacuo. The residue was taken up in toluene (50 mL) and all solvents were removed in vacuo (2×). The solid was dried under high vacuum to afford the TFA salt of the product, N-(5-amino-2-methylpyridin-3-yl)-2-(2-methoxypyridin-3-yl)pyrazolo[5,1-b]thiazole-7-carboxamide, as a tan solid (quantitative yield). The product was used without further purification. LCMS (ESI): mass calcd. for C₁₈H₁₆N₆O₂S, 380.1; m/z found, 381.1 [M+H]⁺; ¹H NMR (400 MHz, DMSO-d6) δ ppm 2.47 (s, 3H), 4.07 (s, 3H), 6.39 (br s, 2H), 7.18 (dd, J=1.00 Hz, 1H), 7.78 (d, J=1.00 Hz, 1H), 7.85 (d, J=1.00 Hz, 1H), 8.23-8.30 (m, 2H), 8.60 (s, 1H), 9.04 (s, 1H), 10.15 (s, 1H).

### Step c: (R)—N-(5-(2-(1-isopropylpyrrolidin-2-yl)acetamido)-2-methylpyridin-3-yl)-2-(2-methoxypyridin-3-yl)pyrazolo[5,1-b]thiazole-7-carboxamide

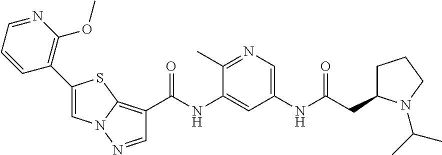 [SMILES: COc1ncccc1-c1cn2ncc(C(=O)Nc3cc(NC(=O)C[C@H]4CCCN4C(C)C)cnc3C)c2s1]

To a suspension of N-(5-amino-2-methylpyridin-3-yl)-2-(2-methoxypyridin-3-yl)pyrazolo[5,1-b]thiazole-7-carboxamide TFA (100 mg, 0.202 mmol) and lithium (R)-2-(1-isopropylpyrrolidin-2-yl)acetate (53.75 mg, 0.30 mmol) in pyridine (3 mL) was added EDCI (58.16 mg, 0.30 mmol). The reaction was stirred at 25° C. for 26 h. All solvents were removed in vacuo. The residue was taken up in DMSO (1.5 mL) and was purified by HPLC (H₂O/ACN, 0.16% NH₄OH, XBridge 19×100) to afford the product, (R)—N-(5-(2-(1-isopropylpyrrolidin-2-yl)acetamido)-2-methylpyridin-3-yl)-2-(2-methoxypyridin-3-yl)pyrazolo[5,1-b]thiazole-7-carboxamide, as a grey solid (41.6 mg). LCMS (ESI): mass calcd. for C₂₇H₃₁N₇O₃S, 533.2; m/z found, 534.3 [M+H]⁺; ¹H NMR (400 MHz, DMSO-d6) δ ppm 0.93 (d, J=1.00 Hz, 3H) 1.08 (d, J=1.00 Hz, 3H) 1.48-1.60 (m, 1H), 1.60-1.76 (m, 2 H), 1.76-1.90 (m, 1H), 2.22-2.33 (m, 1H), 2.37-2.43 (m, 3H), 2.43-2.49 (m, 1H), 2.52-2.57 (m, 1H), 2.71-2.82 (m, 1H), 2.94 (quin, J=1.00 Hz, 1H), 3.09-3.21 (m, 1H), 4.07 (s, 3H), 7.17 (dd, J=1.00 Hz, 1H), 8.17 (d, J=1.00 Hz, 1H), 8.22-8.28 (m, 2H), 8.48 (d, J=1.00 Hz, 1H), 8.58 (s, 1H), 8.98 (s, 1H), 9.88 (s, 1H), 10.30 (s, 1H).

### Example 310: N-(5-(2-(8-oxa-5-azaspiro[3.5]nonan-5-yl)acetamido)-2-methylpyridin-3-yl)-2-(2-methoxypyridin-3-yl)pyrazolo[5,1-b]thiazole-7-carboxamide

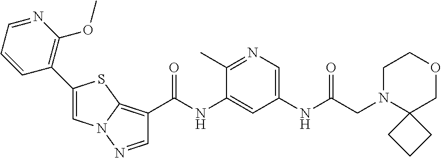 [SMILES: COc1ncccc1-c1cn2ncc(C(=O)Nc3cc(NC(=O)CN4CCOCC45CCC5)cnc3C)c2s1]

### Step a: N-(5-(2-chloroacetamido)-2-methylpyridin-3-yl)-2-(2-methoxypyridin-3-yl)pyrazolo[5,1-b]thiazole-7-carboxamide

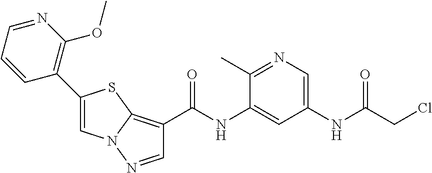 [SMILES: COc1ncccc1-c1cn2ncc(C(=O)Nc3cc(NC(=O)CCl)cnc3C)c2s1]

To a suspension of N-(5-amino-2-methylpyridin-3-yl)-2-(2-methoxypyridin-3-yl)pyrazolo[5,1-b]thiazole-7-carboxamide TFA (1.9 g, 3.8 mmol) in DCM (25 mL) was added Et3N (1.6 mL, 0.728 g/mL, 11.5 mmol). The reaction mixture became more homogeneous and chloroacetyl chloride (0.37 mL, 1.42 g/mL, 4.61 mmol) was added drop wise over 5 min. The reaction was stirred at 25° C. under argon for 3.5 h. All solvents were removed in vacuo. The residue was taken up in MeOH (25 mL) and filtered. The solid collected were washed with MeOH (50 mL), air dried, then dried under high vacuum. The solid was taken up in CH₂Cl₂ (50 mL) and silica gel (5 g) and concentrated in vacuo. The silica gel mesh was loaded on a Redi Sep Rf silica gel cartridge (40 g) eluting with EtOAc over 20 min then MeOH/EtOAc (0-30%) over 10 min to afford the product, N-(5-(2-chloroacetamido)-2-methylpyridin-3-yl)-2-(2-methoxypyridin-3-yl)pyrazolo[5,1-b]thiazole-7-carboxamide, as a tan solid (601.9 mg). LCMS (ESI): mass calcd. for C₂₀H₁₇ClN₆O₃S, 456.1; m/z found, 457.1 [M+H]⁺; ¹H NMR (400 MHz, DMSO-d6) δ ppm 2.43 (s, 3H), 4.08 (s, 3H), 4.31 (s, 2H), 7.17 (dd, J=1.00 Hz, 1H), 8.20 (d, J=1.00 Hz, 1H), 8.22-8.29 (m, 2H), 8.52 (d, J=1.00 Hz, 1H), 8.58 (s, 1H), 9.00 (s, 1H), 9.90 (s, 1H), 10.54 (s, 1H).

### Step b: N-(5-(2-(8-oxa-5-azaspiro[3.5]nonan-5-yl)acetamido)-2-methylpyridin-3-yl)-2-(2-methoxypyridin-3-yl)pyrazolo[5,1-b]thiazole-7-carboxamide

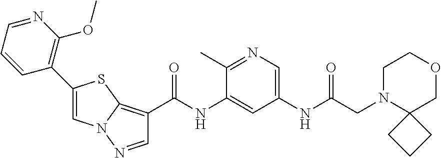 [SMILES: COc1ncccc1-c1cn2ncc(C(=O)Nc3cc(NC(=O)CN4CCOCC45CCC5)cnc3C)c2s1]

To a solution of N-(5-(2-chloroacetamido)-2-methylpyridin-3-yl)-2-(2-methoxypyridin-3-yl)pyrazolo[5,1-b]thiazole-7-carboxamide (60 mg, 0.13 mmol) and 8-oxa-5-azaspiro[3.5]nonane (20 mg, 0.16 mmol) in DMF (2 mL) was added K₂CO₃ (72.6 mg, 0.53 mmol). The reaction was heated at 50° C. for 18.5 h. The reaction was filtered through an Acrodisc CR 13 mm syringe filter with 0.45 mm PTFE membrane. The crude reaction mixture was purified by HPLC (H₂O/ACN, 0.16% NH₄OH, XBridge 19×100) to give the product, N-(5-(2-(8-oxa-5-azaspiro[3.5]nonan-5-yl)acetamido)-2-methylpyridin-3-yl)-2-(2-methoxypyridin-3-yl)pyrazolo[5,1-b]thiazole-7-carboxamide, as an off-white solid (36.3 mg). LCMS (ESI): mass calcd. for C₂₇H₂₉N₇O₄S, 547.2; m/z found, 548.3 [M+H]⁺; ¹H NMR (400 MHz, DMSO-d6) δ ppm 1.60-1.76 (m, 4H), 2.04-2.19 (m, 2H), 2.41 (s, 3H), 2.51-2.58 (m, 2H), 3.16 (d, J=1.00 Hz, 1H), 3.32-3.39 (m, 1H), 3.59 (br s, 2H), 3.67 (t, J=1.00 Hz, 2H), 4.05 (s, 2H), 4.07-4.13 (m, 1H), 7.17 (dd, J=1.00 Hz, 1H), 8.13-8.29 (m, 3H), 8.58 (s, 1H), 8.62 (d, J=1.00 Hz, 1H), 8.98 (s, 1H), 9.78 (s, 1H), 9.90 (s, 1H).

### Example 311: (S)—N-(5-(2-(2-ethylmorpholino)acetamido)-2-methylpyridin-3-yl)-2-(2-methoxypyridin-3-yl)pyrazolo[5,1-b]thiazole-7-carboxamide

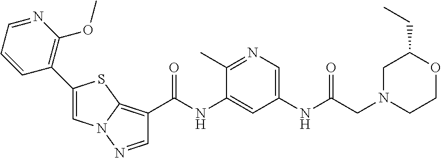 [SMILES: CC[C@H]1CN(CC(=O)Nc2cnc(C)c(NC(=O)c3cnn4cc(-c5cccnc5OC)sc34)c2)CCO1]

To a solution of N-(5-(2-chloroacetamido)-2-methylpyridin-3-yl)-2-(2-methoxypyridin-3-yl)pyrazolo[5,1-b]thiazole-7-carboxamide (60 mg, 0.13 mmol) and (R)-2-ethylmorpholine (18.15 mg, 0.16 mmol) in DMF (2 mL) was added K₂CO₃ (72.6 mg, 0.53 mmol). The reaction was heated at 50° C. for 18 h. The reaction was filtered through an Acrodisc CR 13 mm syringe filter with 0.45 mm PTFE membrane. The crude reaction mixture was purified by HPLC (H₂O/ACN, 0.16% NH₄OH, XBridge 19×100) to afford the product, (S)—N-(5-(2-(2-ethylmorpholino)acetamido)-2-methylpyridin-3-yl)-2-(2-methoxypyridin-3-yl)pyrazolo[5,1-b]thiazole-7-carboxamide, as an off-white solid (10.3 mg). LCMS (ESI): mass calcd. for C₂₆H₂₉N₇O₄S, 535.2; m/z found, 536.3 [M+H]⁺; ¹H NMR (400 MHz, DMSO-d6) δ ppm 0.87 (t, J=1.00 Hz, 3H) 1.32-1.47 (m, 2H), 1.95 (t, J=1.00 Hz, 1H), 2.24 (dt, J=1.00 Hz, 1H), 2.40 (s, 3H), 2.72 (d, J=1.00 Hz, 1H), 2.80 (d, J=1.00 Hz, 1H), 3.15 (s, 2H), 3.35-3.47 (m, 1H), 3.59 (dt, J=1.00 Hz, 1H), 3.77 (d, J=1.00 Hz, 1H), 4.05 (s, 2H), 4.07-4.12 (m, 1H), 7.16 (dd, J=1.00 Hz, 1H), 8.20 (d, J=1.00 Hz, 1H), 8.24 (d, J=1.00 Hz, 1H), 8.24-8.27 (m, 1H), 8.57 (s, 2H), 8.98 (s, 1H), 9.88 (s, 1H), 9.96 (s, 1H).

### Examples 312: 2-(2-hydroxypyridin-3-yl)-N-(2-methyl-5-(2-(pyrrolidin-1-yl)acetamido)pyridin-3-yl)pyrazolo[5,1-b]thiazole-7-carboxamide and Example 313: 2-(2-methoxypyridin-3-yl)-N-(2-methyl-5-(2-(pyrrolidin-1-yl)acetamido)pyridin-3-yl)pyrazolo[5,1-b]thiazole-7-carboxamide

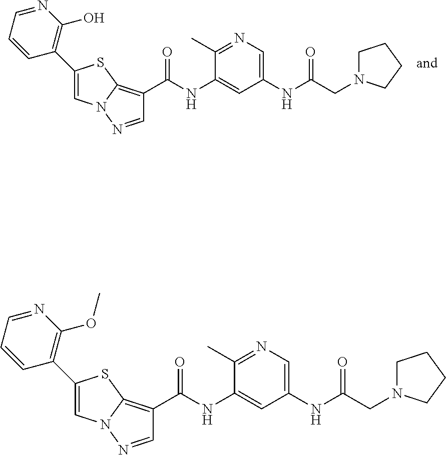 [SMILES: Cc1ncc(NC(=O)CN2CCCC2)cc1NC(=O)c1cnn2cc(-c3cccnc3O)sc12 ; COc1ncccc1-c1cn2ncc(C(=O)Nc3cc(NC(=O)CN4CCCC4)cnc3C)c2s1]

To a mixture of N-(5-amino-2-methylpyridin-3-yl)-2-(2-methoxypyridin-3-yl)pyrazolo[5,1-b]thiazole-7-carboxamide hydrochloride (50 mg, 0.12 mmol), N-(5-amino-2-methylpyridin-3-yl)-2-(2-hydroxypyridin-3-yl)pyrazolo[5,1-b]thiazole-7-carboxamide hydrochloride (45.63 mg, 0.11 mmol), and pyrrolidin-1-ylacetic acid (34 mg, 0.26 mmol) in pyridine (2 mL) was added EDCI (57.5 mg, 0.3 mmol). The reaction was stirred at 25° C. for 21.5 h. All solvents were removed in vacuo. The residue was taken up in DMSO (1.5 mL) and was purified by HPLC (H₂O/ACN, 0.16% NH₄OH, XBridge 19×100) to afford, 2-(2-hydroxypyridin-3-yl)-N-(2-methyl-5-(2-(pyrrolidin-1-yl)acetamido)pyridin-3-yl)pyrazolo[5,1-b]thiazole-7-carboxamide as a tan solid (16.9 mg); LCMS (ESI): mass calcd. for C₂₃H₂₃N₇O₃S, 477.2; m/z found, 478.2 [M+H]⁺; ¹H NMR (400 MHz, DMSO-d6) δ ppm 1.75 (quin, J=1.00 Hz, 4H), 2.41 (s, 3H), 2.60 (quin, J=1.00 Hz, 4H), 3.26 (s, 2H), 6.44 (t, J=1.00 Hz, 1H), 7.54 (dd, J=1.00 Hz, 1H), 8.18 (d, J=1.00 Hz, 1H), 8.23 (dd, J=1.00 Hz, 1H), 8.53 (s, 1H), 8.59 (d, J=1.00 Hz, 1H) 9.02 (s, 1H), 9.80 (s, 1H), 9.94 (s, 1H), 12.35 (s, 1H) and 2-(2-methoxypyridin-3-yl)-N-(2-methyl-5-(2-(pyrrolidin-1-yl)acetamido)pyridin-3-yl)pyrazolo[5,1-b]thiazole-7-carboxamide as a grey solid (23.5 mg); LCMS (ESI): mass calcd. for C₂₄H₂₅N₇O₃S, 491.2; m/z found, 492.2 [M+H]⁺; ¹H NMR (400 MHz, DMSO-d6) δ ppm 1.69-1.83 (m, 4H), 2.40 (s, 3H), 2.55-2.66 (m, 4H), 3.26 (s, 2H), 4.07 (s, 3H), 7.18 (dd, J=1.00 Hz, 1H), 8.15-8.29 (m, 3H), 8.53-8.63 (m, 2H), 8.99 (s, 1H), 9.87 (s, 1H), 9.95 (s, 1H), respectively.

### Examples 314: 2-(2-hydroxypyridin-3-yl)-N-(5-(1-isopropylpiperidine-2-carboxamido)-2-methylpyridin-3-yl)pyrazolo[5,1-b]thiazole-7-carboxamide and Example 315: N-(5-(1-isopropylpiperidine-2-carboxamido)-2-methylpyridin-3-yl)-2-(2-methoxypyridin-3-yl)pyrazolo[5,1-b]thiazole-7-carboxamide

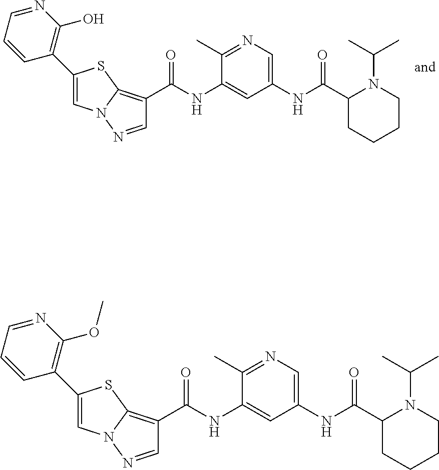 [SMILES: Cc1ncc(NC(=O)C2CCCCN2C(C)C)cc1NC(=O)c1cnn2cc(-c3cccnc3O)sc12 ; COc1ncccc1-c1cn2ncc(C(=O)Nc3cc(NC(=O)C4CCCCN4C(C)C)cnc3C)c2s1]

To a mixture of N-(5-amino-2-methylpyridin-3-yl)-2-(2-methoxypyridin-3-yl)pyrazolo[5,1-b]thiazole-7-carboxamide hydrochloride (50 mg, 0.12 mmol), N-(5-amino-2-methylpyridin-3-yl)-2-(2-hydroxypyridin-3-yl)pyrazolo[5,1-b]thiazole-7-carboxamide hydrochloride (45.63 mg, 0.11 mmol), and 1-isopropylpiperidine-3-carboxylic acid (45.18 mg, 0.26 mmol)) in pyridine (2 mL) was added EDCI (57.5 mg, 0.3 mmol). The reaction was stirred at 25° C. for 21 h. All solvents were removed in vacuo. The residue was taken up in DMSO (1.5 mL) and was purified by HPLC (H₂O/ACN, 0.16% NH₄OH, XBridge 19×100) to afford 2-(2-hydroxypyridin-3-yl)-N-(5-(1-isopropylpiperidine-2-carboxamido)-2-methylpyridin-3-yl)pyrazolo[5,1-b]thiazole-7-carboxamide as a tan solid (23.9 mg); LCMS (ESI): mass calcd. for C₂₆H₂₉N₇O₃S, 519.2; m/z found, 520.2 [M+H]⁺; ¹H NMR (400 MHz, DMSO-d6) δ ppm 0.98 (d, J=1.00 Hz, 6H), 1.37-1.53 (m, 2H), 1.62-1.75 (m, 1H), 1.75-1.86 (m, 1H), 2.14 (t, J=1.00 Hz, 1H), 2.29 (t, J=1.00 Hz, 1H), 2.40 (s, 3H), 2.52-2.64 (m, 1H), 2.64-2.81 (m, 2H), 2.81-2.91 (m, 1H), 6.44 (t, J=1.00 Hz, 1H), 7.55 (d, J=1.00 Hz, 1H), 8.13 (d, J=1.00 Hz, 1H), 8.23 (dd, J=1.00 Hz, 1H), 8.51 (d, J=1.00 Hz, 1H), 8.54 (s, 1H), 9.03 (s, 1H), 9.80 (s, 1H), 10.21 (s, 1H), 12.35 (s, 1H), and N-(5-(1-isopropylpiperidine-2-carboxamido)-2-methylpyridin-3-yl)-2-(2-methoxypyridin-3-yl)pyrazolo[5,1-b]thiazole-7-carboxamide as a grey solid (44.0 mg); LCMS (ESI): mass calcd. for C₂₇H₃₁N₇O₃S, 533.2; m/z found, 534.2 [M+H]⁺; ¹H NMR (400 MHz, DMSO-d6) δ ppm 0.98 (d, J=1.00 Hz, 6H), 1.38-1.52 (m, 2H), 1.62-1.75 (m, 1H), 1.75-1.88 (m, 1H), 2.14 (t, J=1.00 Hz, 1H), 2.29 (t, J=1.00 Hz, 1H), 2.41 (s, 3H), 2.53-2.59 (m, 1H), 2.62-2.80 (m, 2H), 2.86 (d, J=1.00 Hz, 1H), 4.07 (s, 3H), 7.17 (t, J=1.00 Hz, 1H), 8.13 (s, 1H), 8.20-8.32 (m, 2H), 8.48 (s, 1H), 8.61 (s, 1H), 8.99 (s, 1H), 9.87 (s, 1H), 10.22 (s, 1H), respectively.

### Examples 316: N-(5-((2S,4S)-1,4-dimethylpyrrolidine-2-carboxamido)-2-methylpyridin-3-yl)-2-(2-hydroxypyridin-3-yl)pyrazolo[5,1-b]thiazole-7-carboxamide and Example 317: N-(5-((2S,4S)-1,4-dimethylpyrrolidine-2-carboxamido)-2-methylpyridin-3-yl)-2-(2-methoxypyridin-3-yl)pyrazolo[5,1-b]thiazole-7-carboxamide

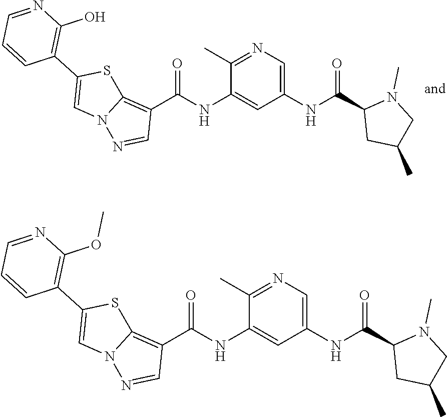 [SMILES: Cc1ncc(NC(=O)[C@@H]2C[C@H](C)CN2C)cc1NC(=O)c1cnn2cc(-c3cccnc3O)sc12 ; COc1ncccc1-c1cn2ncc(C(=O)Nc3cc(NC(=O)[C@@H]4C[C@H](C)CN4C)cnc3C)c2s1]

To a mixture of N-(5-amino-2-methylpyridin-3-yl)-2-(2-methoxypyridin-3-yl)pyrazolo[5,1-b]thiazole-7-carboxamide hydrochloride (50 mg, 0.12 mmol), N-(5-amino-2-methylpyridin-3-yl)-2-(2-hydroxypyridin-3-yl)pyrazolo[5,1-b]thiazole-7-carboxamide hydrochloride (45.63 mg, 0.11 mmol), and (2S,4S)-1,4-dimethylpyrrolidine-2-carboxylic acid (37.78 mg, 0.26 mmol)) in pyridine (2 mL) was added EDCI (57.48 mg, 0.3 mmol). The reaction was stirred at 25° C. for 21 h. All solvents were removed in vacuo. The residue was taken up in DMSO (1.5 mL) and was purified by HPLC (H₂O/ACN, 0.16% NH₄OH, XBridge 19×100) to afford N-(5-((2S,4S)-1,4-dimethylpyrrolidine-2-carboxamido)-2-methylpyridin-3-yl)-2-(2-hydroxypyridin-3-yl)pyrazolo[5,1-b]thiazole-7-carboxamide as a tan solid (31.5 mg); LCMS (ESI): mass calcd. for C₂₄H₂₅N₇O₃S, 491.2; m/z found, 492.2 [M+H]⁺; ¹H NMR (400 MHz, DMSO-d6) δ ppm 1.06 (d, J=1.00 Hz, 3H), 1.39-1.48 (m, 1H), 2.20-2.26 (m, 1H), 2.33 (s, 3H), 2.35-2.40 (m, 1H), 2.42 (s, 3H), 2.57 (dd, J=1.00 Hz, 1H), 2.78 (dd, J=1.00 Hz, 1H), 3.00 (t, J=1.00 Hz, 1H), 6.43 (t, J=1.00 Hz, 1H), 7.54 (dd, J=1.00 Hz, 1H), 8.19-8.26 (m, 2H), 8.54 (s, 1H), 8.63 (d, J=1.00 Hz, 1H), 9.04 (s, 1H), 9.80 (s, 1H), 9.94 (s, 1H), 12.37 (br s, 1H) and N-(5-((2S,4S)-1,4-dimethylpyrrolidine-2-carboxamido)-2-methylpyridin-3-yl)-2-(2-methoxypyridin-3-yl)pyrazolo[5,1-b]thiazole-7-carboxamide as a grey solid (25.4 mg); LCMS (ESI): mass calcd. for C₂₅H₂₇N₇O₃S, 505.2; m/z found, 506.2 [M+H]⁺; ¹H NMR (400 MHz, DMSO-d6) δ ppm 1.06 (d, J=1.00 Hz, 3H), 1.34-1.51 (m, 1 H), 2.17-2.29 (m, 1H), 2.33 (s, 3H), 2.36-2.40 (m, 1H), 2.42 (s, 3H), 2.58 (t, J=1.00 Hz, 1H), 2.73-2.85 (m, 1H), 2.94-3.09 (m, 1H), 3.16 (d, J=1.00 Hz, 3H), 7.17 (dd, J=1.00 Hz, 1H), 8.17-8.33 (m, 3H), 8.55 (s, 1H), 8.65 (s, 1H), 8.98 (s, 1H), 9.87 (s, 1H), 9.97 (s, 1H), respectively.

### Example 318: (R)-2-(2-methoxypyridin-3-yl)-N-(2-methyl-5-(2-(2-methylmorpholino)acetamido)pyridin-3-yl)pyrazolo[5,1-b]thiazole-7-carboxamide

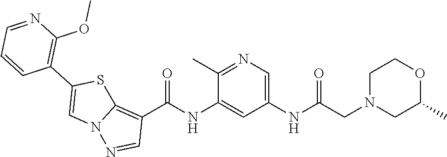 [SMILES: COc1ncccc1-c1cn2ncc(C(=O)Nc3cc(NC(=O)CN4CCO[C@H](C)C4)cnc3C)c2s1]

To a solution of N-(5-(2-chloroacetamido)-2-methylpyridin-3-yl)-2-(2-methoxypyridin-3-yl)pyrazolo[5,1-b]thiazole-7-carboxamide (60 mg, 0.13 mmol) and (R)-2-methylmorpholine hydrochloride (15.94 mg, 0.12 mmol) in DMF (2 mL) was added K₂CO₃ (72.6 mg, 0.53 mmol). The reaction was capped and heated at 50° C. for 22.5 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, (R)-2-(2-methoxypyridin-3-yl)-N-(2-methyl-5-(2-(2-methylmorpholino)acetamido)pyridin-3-yl)pyrazolo[5,1-b]thiazole-7-carboxamide, as a tan solid (6.8 mg). LCMS (ESI): mass calcd. for C₂₅H₂₇N₇O₄S, 521.2; m/z found, 522.2 [M+H]⁺; ¹H NMR (400 MHz, DMSO-d6) δ ppm 1.05 (d, J=1.00 Hz, 3H), 1.93 (t, J=1.00 Hz, 1H), 2.24 (dt, J=1.00 Hz, 1H), 2.41 (s, 3H), 2.71 (d, J=1.00 Hz, 1H), 2.79 (d, J=1.00 Hz, 1H), 3.16 (s, 2H), 3.55-3.68 (m, 2H), 3.74 (d, J=1.00 Hz, 1H), 4.08 (s, 3H), 7.16 (dd, J=1.00 Hz, 1H), 8.18 (s, 1H), 8.22-8.32 (m, 2H), 8.59 (s, 2H), 8.99 (s, 1H), 9.88 (s, 1H), 9.97 (s, 1H).

### Example 319: N-(5-(2-(2,2-dimethylmorpholino)acetamido)-2-methylpyridin-3-yl)-2-(2-methoxypyridin-3-yl)pyrazolo[5,1-b]thiazole-7-carboxamide

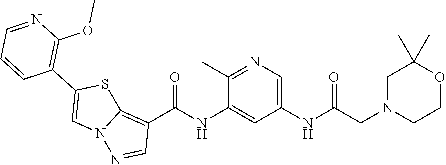 [SMILES: COc1ncccc1-c1cn2ncc(C(=O)Nc3cc(NC(=O)CN4CCOC(C)(C)C4)cnc3C)c2s1]

To a solution of N-(5-(2-chloroacetamido)-2-methylpyridin-3-yl)-2-(2-methoxypyridin-3-yl)pyrazolo[5,1-b]thiazole-7-carboxamide (60 mg, 0.13 mmol) and 2,2-dimethylmorpholine (18.15 mg, 0.16 mmol) in DMF (2 mL) was added K₂CO₃ (72.6 mg, 0.53 mmol). The reaction was capped and heated at 50° C. for 22.5 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, N-(5-(2-(2,2-dimethylmorpholino)acetamido)-2-methylpyridin-3-yl)-2-(2-methoxypyridin-3-yl)pyrazolo[5,1-b]thiazole-7-carboxamide, as a tan solid (21.5 mg). LCMS (ESI): mass calcd. for C₂₆H₂₉N₇O₄S, 535.2; m/z found, 536.2 [M+H]⁺; ¹H NMR (400 MHz, DMSO-d6) δ ppm 1.21 (s, 6H), 2.26 (s, 2H), 2.40 (s, 3H), 2.45 (t, J=1.00 Hz, 2H), 3.11 (s, 2H), 3.68 (t, J=1.00 Hz, 2H), 4.07 (s, 3H), 7.17 (dd, J=1.00 Hz, 1H), 8.19 (d, J=1.00 Hz, 1H), 8.22-8.30 (m, 2H), 8.53 (d, J=1.00 Hz, 1H), 8.59 (s, 1H), 8.99 (s, 1H), 9.86 (s, 1H), 9.91 (s, 1H).

### Example 320: N-(5-(2-(2-oxa-7-azaspiro[4.4]nonan-7-yl)acetamido)-2-methylpyridin-3-yl)-2-(2-methoxypyridin-3-yl)pyrazolo[5,1-b]thiazole-7-carboxamide

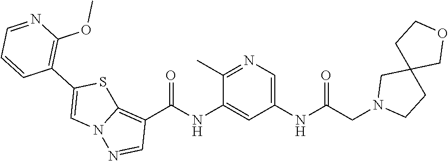

To a solution of N-(5-(2-chloroacetamido)-2-methylpyridin-3-yl)-2-(2-methoxypyridin-3-yl)pyrazolo[5,1-b]thiazole-7-carboxamide (60 mg, 0.13 mmol) and 2-oxa-7-azaspiro[4.4]nonane (20 mg, 0.16 mmol) in DMF (2 mL) was added K₂CO₃ (72.6 mg, 0.53 mmol). The reaction was capped and heated at 50° C. for 23.5 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, N-(5-(2-(2-oxa-7-azaspiro[4.4]nonan-7-yl)acetamido)-2-methylpyridin-3-yl)-2-(2-methoxypyridin-3-yl)pyrazolo[5,1-b]thiazole-7-carboxamide, as a tan solid (42.2 mg). LCMS (ESI): mass calcd. for C₂₇H₂₉N₇O₄S, 547.2; m/z found, 548.2 [M+H]⁺; ¹H NMR (400 MHz, DMSO-d6) δ ppm 1.79 (t, J=1.00 Hz, 2H), 1.83-1.98 (m, 2H), 2.41 (s, 3H), 2.56 (d, J=1.00 Hz, 1H), 2.68 (q, J=1.00 Hz, 2H), 2.77 (q, J=1.00 Hz, 1H), 3.28 (s, 2H), 3.47 (d, J=1.00 Hz, 1H), 3.61 (d, J=1.00 Hz, 1H), 3.72 (sxt, J=1.00 Hz, 2H), 4.06 (s, 3H), 7.17 (dd, J=1.00 Hz, 1H), 8.19 (d, J=1.00 Hz, 1H), 8.22-8.28 (m, 2H), 8.56 (d, J=1.00 Hz, 1H), 8.59 (s, 1H), 8.99 (s, 1H), 9.89 (s, 1H), 9.94 (s, 1H).

### Example 321: N-(5-(2-(5-azaspiro[2.5]octan-5-yl)acetamido)-2-methylpyridin-3-yl)-2-(2-methoxypyridin-3-yl)pyrazolo[5,1-b]thiazole-7-carboxamide

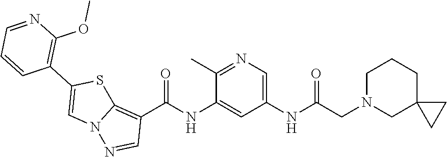 [SMILES: COc1ncccc1-c1cn2ncc(C(=O)Nc3cc(NC(=O)CN4CCCC5(CC5)C4)cnc3C)c2s1]

To a solution of N-(5-(2-chloroacetamido)-2-methylpyridin-3-yl)-2-(2-methoxypyridin-3-yl)pyrazolo[5,1-b]thiazole-7-carboxamide (60 mg, 0.13 mmol) and 5-azaspiro[2.5]octane hydrochloride (23.27 mg, 0.16 mmol) in DMF (2 mL) was added K₂CO₃ (72.6 mg, 0.53 mmol). The reaction was capped and heated at 50° C. for 23.5 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, N-(5-(2-(5-azaspiro[2.5]octan-5-yl)acetamido)-2-methylpyridin-3-yl)-2-(2-methoxypyridin-3-yl)pyrazolo[5,1-b]thiazole-7-carboxamide, as a tan solid (33.8 mg). LCMS (ESI): mass calcd. for C₂₇H₂₉N₇O₃S, 531.2; m/z found, 532.2 [M+H]⁺; ¹H NMR (400 MHz, DMSO-d6) δ ppm 0.30 (dt, J=1.00 Hz, 4H), 1.29 (t, J=1.00 Hz, 2H), 1.67 (quin, J=1.00 Hz, 2H), 2.25 (s, 2H), 2.41 (s, 3H), 2.57 (t, J=1.00 Hz, 2H), 3.11 (s, 2H), 4.07 (s, 3H), 7.17 (dd, J=1.00 Hz, 1H), 8.17 (d, J=1.00 Hz, 1H), 8.21-8.29 (m, 2H), 8.51 (d, J=1.00 Hz, 1H), 8.58 (s, 1H), 8.99 (s, 1H), 9.87 (s, 1H), 9.91 (s, 1H).

### Examples 322: (S)-2-(2-hydroxypyridin-3-yl)-N-(5-(2-(1-isopropylpyrrolidin-2-yl)acetamido)-2-methylpyridin-3-yl)pyrazolo[5,1-b]thiazole-7-carboxamide and Example 323 (S)—N-(5-(2-(1-isopropylpyrrolidin-2-yl)acetamido)-2-methylpyridin-3-yl)-2-(2-methoxypyridin-3-yl)pyrazolo[5,1-b]thiazole-7-carboxamide

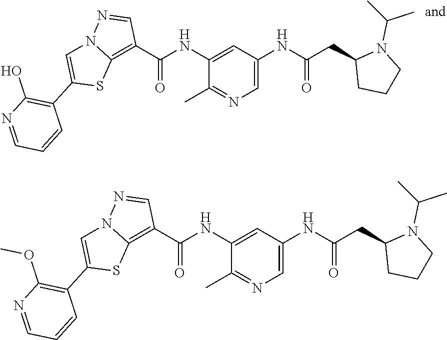

To a mixture of N-(5-amino-2-methylpyridin-3-yl)-2-(2-methoxypyridin-3-yl)pyrazolo[5,1-b]thiazole-7-carboxamide hydrochloride (50 mg, 0.12 mmol) and N-(5-amino-2-methylpyridin-3-yl)-2-(2-hydroxypyridin-3-yl)pyrazolo[5,1-b]thiazole-7-carboxamide hydrochloride (48.32 mg, 0.12 mmol) and lithium (S)-2-(1-isopropylpyrrolidin-2-yl)acetate (46.75 mg, 0.26 mmol) in pyridine (2 mL) was added EDCI (57.5 mg, 0.3 mmol). The reaction was stirred at 25° C. for 21 h. All solvents were removed in vacuo. The residue was taken up in DMSO (2 mL) and was purified by HPLC (H₂O/ACN, 0.16% NH₄OH, XBridge 19×100) to afford two products, (S)-2-(2-hydroxypyridin-3-yl)-N-(5-(2-(1-isopropylpyrrolidin-2-yl)acetamido)-2-methylpyridin-3-yl)pyrazolo[5,1-b]thiazole-7-carboxamide, as a tan solid (28.8 mg); LCMS (ESI): mass calcd. for C₂₆H₂₉N₇O₃S, 519.2; m/z found, 520.3 [M+H]⁺; ¹H NMR (400 MHz, DMSO-d6) δ ppm 0.94 (d, J=1.00 Hz, 3H), 1.06 (d, J=1.00 Hz, 3H), 1.46-1.60 (m, 1H), 1.60-1.76 (m, 2H), 1.76-1.90 (m, 1H), 2.27 (dd, J=1.00 Hz, 1H), 2.39 (s, 3H), 2.52-2.57 (m, 1H), 2.72-2.83 (m, 1H), 2.94 (spt, J=1.00 Hz, 1H), 3.08-3.16 (m, 1H), 3.18 (s, 2H), 6.42 (t, J=1.00 Hz, 1H), 7.55 (dd, J=1.00 Hz, 1H), 8.13 (d, J=1.00 Hz, 1H), 8.21 (dd, J=1.00 Hz, 1H), 8.49 (d, J=1.00 Hz, 1H), 8.53 (s, 1H), 9.01 (s, 1H), 9.80 (s, 1H), 10.30 (s, 1H), and (S)—N-(5-(2-(1-isopropylpyrrolidin-2-yl)acetamido)-2-methylpyridin-3-yl)-2-(2-methoxypyridin-3-yl)pyrazolo[5,1-b]thiazole-7-carboxamide, as a tan solid (1.8 mg); LCMS (ESI): mass calcd. for C₂₇H₃₁N₇O₃S, 533.2; m/z found, 534.2 [M+H]⁺;

### Example 324: N-(5-(2-(5-azaspiro[2.4]heptan-5-yl)acetamido)-2-methylpyridin-3-yl)-2-(2-methoxypyridin-3-yl)pyrazolo[5,1-b]thiazole-7-carboxamide

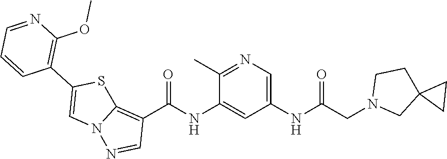

To a solution of N-(5-(2-chloroacetamido)-2-methylpyridin-3-yl)-2-(2-methoxypyridin-3-yl)pyrazolo[5,1-b]thiazole-7-carboxamide (60 mg, 0.13 mmol) and 5-azaspiro[2.4]heptane (15.31 mg, 0.16 mmol) in DMF (1.2 mL) was added K₂CO₃ (72.6 mg, 0.5 mmol). The reaction was heated at 50° C. for 19 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, N-(5-(2-(5-azaspiro[2.4]heptan-5-yl)acetamido)-2-methylpyridin-3-yl)-2-(2-methoxypyridin-3-yl)pyrazolo[5,1-b]thiazole-7-carboxamide, as an off-white solid (23.5 mg). LCMS (ESI): mass calcd. for C₂₆H₂₇N₇O₃S, 517.2; m/z found, 518.1 [M+H]⁺; ¹H NMR (400 MHz, DMSO-d6) δ ppm 0.53 (dd, J=1.00 Hz, 4H), 1.77 (t, J=1.00 Hz, 2H), 2.40 (s, 3H), 2.59 (s, 2H), 2.79 (t, J=1.00 Hz, 2H), 3.26 (s, 2H), 4.07 (s, 3H), 7.16 (dd, J=1.00 Hz, 1H), 8.19 (d, J=1.00 Hz, 1H), 8.22-8.30 (m, 2H), 8.58 (s, 2H), 8.99 (s, 1H), 9.89 (s, 1H), 10.00 (s, 1H).

### Example 325: (S)-2-(1-(2-methoxyethyl)-1H-pyrazol-4-yl)-N-(2-methyl-5-(2-(2-methylpyrrolidin-1-yl)acetamido)pyridin-3-yl)pyrazolo[5,1-b]thiazole-7-carboxamide

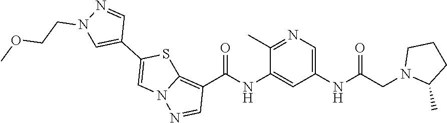 [SMILES: COCCn1cc(-c2cn3ncc(C(=O)Nc4cc(NC(=O)CN5CCC[C@@H]5C)cnc4C)c3s2)cn1]

### Step a: tert-butyl (5-(2-(1-(2-methoxyethyl)-1H-pyrazol-4-yl)pyrazolo[5,1-b]thiazole-7-carboxamido)-6-methylpyridin-3-yl)carbamate

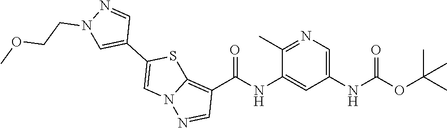 [SMILES: COCCn1cc(-c2cn3ncc(C(=O)Nc4cc(NC(=O)OC(C)(C)C)cnc4C)c3s2)cn1]

To a mixture of tert-butyl (5-(2-bromopyrazolo[5,1-b]thiazole-7-carboxamido)-6-methylpyridin-3-yl)carbamate (250 mg, 0.55 mmol), 1-(2-methoxyethyl)-4-(4,4,5,5-tetramethyl-1,3,2-dioxaborolan-2-yl)-1H-pyrazole (209 mg, 0.83 mmol), and K₂CO₃ (229.16 mg, 1.66 mmol) in 1,4-dioxane (2.4 mL) and water (0.6 mL) was added 1,1′-bis(diphenylphosphino)ferrocene-palladium(II)dichloride dichloromethane complex (90.27 mg, 0.11 mmol). The reaction was thoroughly flushed with argon before being capped and heated at 100° C. for 25 h. The reaction was diluted with MeOH (25 mL) and silica gel (3 g) was added. All solvents were removed in vacuo. The silica gel mesh was loaded on a Redi Sep Rf silica gel cartridge (40 g) eluting with EtOAc Over 10 min then MeOH/EtOAc (0-30%) over 15 min to afford the product, tert-butyl (5-(2-(1-(2-methoxyethyl)-1H-pyrazol-4-yl)pyrazolo[5,1-b]thiazole-7-carboxamido)-6-methylpyridin-3-yl)carbamate, as a tan solid (170 mg). LCMS (ESI): mass calcd. for C₂₃H₇N₇O₄S, 497.2; m/z found, 498.2 [M+H]⁺.

### Step b: N-(5-amino-2-methylpyridin-3-yl)-2-(1-(2-methoxyethyl)-1H-pyrazol-4-yl)pyrazolo[5,1-b]thiazole-7-carboxamide

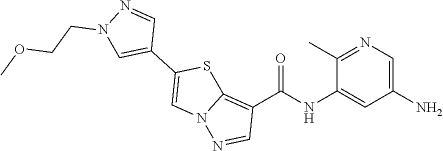 [SMILES: COCCn1cc(-c2cn3ncc(C(=O)Nc4cc(N)cnc4C)c3s2)cn1]

To a solution of tert-butyl (5-(2-(1-(2-methoxyethyl)-1H-pyrazol-4-yl)pyrazolo[5,1-b]thiazole-7-carboxamido)-6-methylpyridin-3-yl)carbamate (170 mg, 0.34 mmol) in DCM (2 mL) was added HCl (4M in dioxane) (0.43 mL, 4 M, 1.71 mmol). The reaction mixture was stirred at 25° C. for 96 h. All solvents were removed in vacuo to afford the HCl salt of the product, N-(5-amino-2-methylpyridin-3-yl)-2-(1-(2-methoxyethyl)-1H-pyrazol-4-yl)pyrazolo[5,1-b]thiazole-7-carboxamide, as a tan solid (quantitative yield). The product was used without any further purification. LCMS (ESI): mass calcd. for C₁₈H₁₉N₇O₂S, 397.1; m/z found, 398.2 [M+H]⁺; ¹H NMR (400 MHz, DMSO-d6) δ ppm 2.52 (s, 3H), 3.26 (s, 3H), 3.73 (t, J=1.00 Hz, 2H), 4.30 (t, J=1.00 Hz, 2H), 6.47 (br s, 3H), 7.77 (d, J=1.00 Hz, 1H), 7.84 (d, J=1.00 Hz, 1H), 7.91 (s, 1H), 8.23 (s, 1H), 8.62 (s, 1H), 8.65 (s, 1H), 10.32 (s, 1H).

### Step c: N-(5-(2-chloroacetamido)-2-methylpyridin-3-yl)-2-(1-(2-methoxyethyl)-1H-pyrazol-4-yl)pyrazolo[5,1-b]thiazole-7-carboxamide

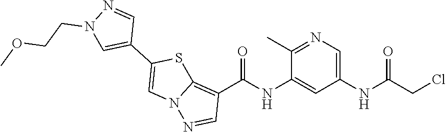 [SMILES: COCCn1cc(-c2cn3ncc(C(=O)Nc4cc(NC(=O)CCl)cnc4C)c3s2)cn1]

To a solution of N-(5-amino-2-methylpyridin-3-yl)-2-(1-(2-methoxyethyl)-1H-pyrazol-4-yl)pyrazolo[5,1-b]thiazole-7-carboxamide hydrochloride (148.40 mg, 0.34 mmol) and Et₃N (0.14 mL, 0.728 g/mL, 1.03 mmol) in DCM (2 mL) was added chloroacetyl chloride (0.033 mL, 1.42 g/mL, 0.41 mmol). The reaction was stirred at 25° C. for 4.5 h. Silica gel (1 g) was added and all solvents were removed in vacuo. The silica gel mesh was loaded on a Redi Sep Rf silica gel cartridge (24 g) eluting with MeOH/CH₂Cl₂ (0-30%) over 15 min to afford he product, N-(5-(2-chloroacetamido)-2-methylpyridin-3-yl)-2-(1-(2-methoxyethyl)-1H-pyrazol-4-yl)pyrazolo[5,1-b]thiazole-7-carboxamide as a tan solid (161.14 mg). LCMS (ESI): mass calcd. for C₂₀H₂₀ClN₇O₃S, 473.1; m/z found, 474.1 [M+H]⁺; ¹H NMR (400 MHz, DMSO-d6) δ ppm 2.42 (s, 3H), 3.25 (s, 3H), 3.71 (t, J=1.00 Hz, 2H), 4.26-4.34 (m, 4H), 7.91 (s, 1H), 8.14 (d, J=1.00 Hz, 1H), 8.22 (s, 1H), 8.50 (s, 1H), 8.54 (d, J=1.00 Hz, 1H), 8.59 (s, 1H), 9.89 (s, 1H), 10.53 (s, 1H).

### Step d: (S)-2-(1-(2-methoxyethyl)-1H-pyrazol-4-yl)-N-(2-methyl-5-(2-(2-methylpyrrolidin-1-yl)acetamido)pyridin-3-yl)pyrazolo[5,1-b]thiazole-7-carboxamide

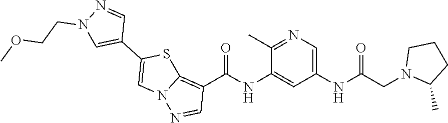 [SMILES: COCCn1cc(-c2cn3ncc(C(=O)Nc4cc(NC(=O)CN5CCC[C@@H]5C)cnc4C)c3s2)cn1]

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol), (S)-(+)-2-methylpyrrolidine (12.94 mg, 0.15 mmol), and K₂CO₃ (69.99 mg, 0.51 mmol) in DMF (2 mL) was heated at 50° C. for 21 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, (S)-2-(1-(2-methoxyethyl)-1H-pyrazol-4-yl)-N-(2-methyl-5-(2-(2-methylpyrrolidin-1-yl)acetamido)pyridin-3-yl)pyrazolo[5,1-b]thiazole-7-carboxamide, as a tan solid (42.4 mg). LCMS (ESI): mass calcd. for C₂₅H₃₀N₈O₃S, 522.2; m/z found, 523.2 [M+H]⁺; ¹H NMR (400 MHz, DMSO-d6) δ ppm 1.07 (d, J=1.00 Hz, 3H), 1.31-1.48 (m, 1H), 1.59-1.71 (m, 1H), 1.71-1.84 (m, 1H), 1.84-2.01 (m, 1H), 2.33 (q, J=1.00 Hz, 1H), 2.41 (s, 3H), 2.51-2.60 (m, 1H), 3.01 (d, J=1.00 Hz, 1H), 3.12 (dt, J=1.00 Hz, 1H), 3.24 (s, 3H), 3.46 (d, J=1.00 Hz, 1H), 3.71 (t, J=1.00 Hz, 2H), 4.30 (t, J=1.00 Hz, 2H), 7.90 (s, 1H), 8.15 (d, J=1.00 Hz, 1H), 8.22 (s, 1H), 8.51 (s, 1H), 8.58 (s, 1H), 8.61 (d, J=1.00 Hz, 1H), 9.82 (s, 1H), 9.90 (s, 1H).

### Example 326: N-(5-(2-(5-azaspiro[2.4]heptan-5-yl)acetamido)-2-methylpyridin-3-yl)-2-(1-(2-methoxyethyl)-1H-pyrazol-4-yl)pyrazolo[5,1-b]thiazole-7-carboxamide

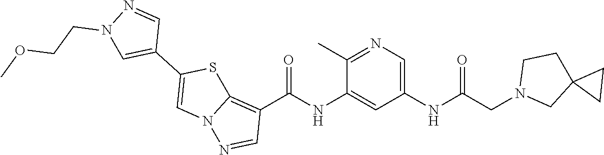 [SMILES: COCCn1cc(-c2cn3ncc(C(=O)Nc4cc(NC(=O)CN5CCC6(CC6)C5)cnc4C)c3s2)cn1]

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol), 5-azaspiro[2.4]heptane (14.76 mg, 0.15 mmol), and K₂CO₃ (69.99 mg, 0.51 mmol) in DMF (2 mL) was heated at 50° C. for 21 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, N-(5-(2-(5-azaspiro[2.4]heptan-5-yl)acetamido)-2-methylpyridin-3-yl)-2-(1-(2-methoxyethyl)-1H-pyrazol-4-yl)pyrazolo[5,1-b]thiazole-7-carboxamide, as a tan solid (39.8 mg). LCMS (ESI): mass calcd. for C₂₆H₃₀N₈O₃S, 534.2; m/z found, 535.2 [M+H]⁺; ¹H NMR (400 MHz, DMSO-d6) δ ppm 0.53 (dd, J=1.00 Hz, 4H), 1.78 (t, J=1.00 Hz, 2H), 2.40 (s, 3H), 2.59 (s, 2H), 2.80 (t, J=1.00 Hz, 2H), 3.23 (s, 3H), 3.28 (s, 2H), 3.72 (t, J=1.00 Hz, 2H), 4.30 (t, J=1.00 Hz, 2H), 7.89 (s, 1H), 8.16 (d, J=1.00 Hz, 1H), 8.22 (s, 1H), 8.51 (s, 1H), 8.58 (d, J=1.00 Hz, 1H), 8.60 (s, 1H), 9.88 (s, 1H), 9.99 (s, 1H).

### Example 327: (R*)-2-(1-(2-methoxyethyl)-1H-pyrazol-4-yl)-N-(2-methyl-5-(2-(3-methylpyrrolidin-1-yl)acetamido)pyridin-3-yl)pyrazolo[5,1-b]thiazole-7-carboxamide and Example 328: (S*)-2-(1-(2-methoxyethyl)-1H-pyrazol-4-yl)-N-(2-methyl-5-(2-(3-methylpyrrolidin-1-yl)acetamido)pyridin-3-yl)pyrazolo[5,1-b]thiazole-7-carboxamide

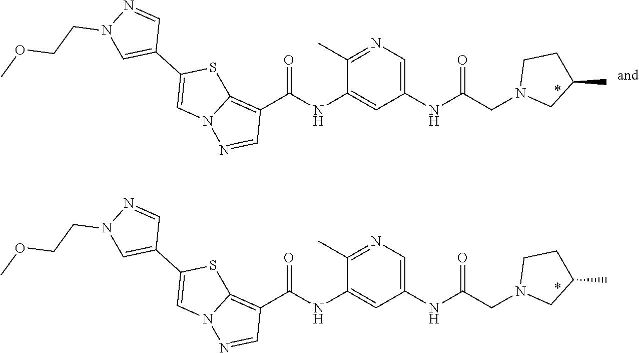

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (80.4 mg, 0.17 mmol), 3-methylpyrrolidine (17.33 mg, 0.20 mmol), and K₂CO₃ (93.78 mg, 0.68 mmol) in DMF (2 mL) was heated at 50° C. for 19 h. Silica gel (5 g) was added to the reaction and all solvents were removed in vacuo. The silica gel mesh was loaded on a Redi Sep Rf silica gel cartridge (24 g) eluting with EtOAc over 5 mim, MeOH/EtOAc (0-30%) over 15 min, then 30% MeOH/EtOAc over 10 min to afford the racemic products (79.6 mg). The products were separated by chiral HPLC (column AD, 2.1×100 mm, ID, 3 μm; 75% CO₂, 25% methanol with 0.1 diisopropylethyl amine, 2 mL/min) to afford two enantiomerically pure products. First eluting product, (R*)-2-(1-(2-methoxyethyl)-1H-pyrazol-4-yl)-N-(2-methyl-5-(2-(3-methylpyrrolidin-1-yl)acetamido)pyridin-3-yl)pyrazolo[5,1-b]thiazole-7-carboxamide, as a tan solid (29.4 mg); LCMS (ESI): mass calcd. for C₂₅H₃₀N₈O₃S, 522.2; m/z found, 523.2 [M+H]⁺; ¹H NMR (400 MHz, DMSO-d6) δ ppm 1.01 (d, J=1.00 Hz, 3H) 1.27-1.39 (m, 2H) 1.90-2.05 (m, 1H) 2.14 (q, J=1.00 Hz, 1H) 2.19-2.30 (m, 1H) 2.40 (s, 3H) 2.54-2.65 (m, 1H) 2.73 (q, J=1.00 Hz, 1H) 2.86 (t, J=1.00 Hz, 1H) 3.22-3.28 (m, 4H) 3.71 (t, J=1.00 Hz, 2H) 4.29 (t, J=1.00 Hz, 2H) 7.90 (s, 1H) 8.14 (s, 1H) 8.22 (s, 1H) 8.49 (s, 1H) 8.60 (s, 2H) 9.93 (br s, 2H) and second eluting product, (S*)-2-(1-(2-methoxyethyl)-1H-pyrazol-4-yl)-N-(2-methyl-5-(2-(3-methylpyrrolidin-1-yl)acetamido)pyridin-3-yl)pyrazolo[5,1-b]thiazole-7-carboxamide, as a tan solid (22.0 mg); LCMS (ESI): mass calcd. for C₂₅H₃₀N₈O₃S, 522.2; m/z found, 523.2 [M+H]⁺; ¹H NMR (400 MHz, DMSO-d6) δ ppm 1.01 (d, J=1.00 Hz, 3H) 1.27-1.39 (m, 2H) 1.90-2.05 (m, 1H) 2.14 (q, J=1.00 Hz, 1H) 2.19-2.30 (m, 1H) 2.40 (s, 3H) 2.54-2.65 (m, 1H) 2.73 (q, J=1.00 Hz, 1H) 2.86 (t, J=1.00 Hz, 1H) 3.22-3.28 (m, 4H) 3.71 (t, J=1.00 Hz, 2H) 4.29 (t, J=1.00 Hz, 2H) 7.90 (s, 1H) 8.14 (s, 1H) 8.22 (s, 1H) 8.49 (s, 1H) 8.60 (s, 2H) 9.93 (br s, 2H).

### Example 329: N-(5-(2-(2,2-dimethylpyrrolidin-1-yl)acetamido)-2-methylpyridin-3-yl)-2-(1-(2-methoxyethyl)-1H-pyrazol-4-yl)pyrazolo[5,1-b]thiazole-7-carboxamide

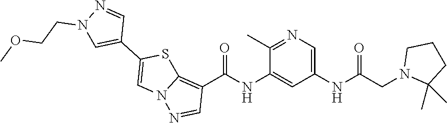

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol), 2,2-dimethylpyrrolidine (15 mg, 0.15 mmol), and K₂CO₃ (70 mg, 0.51 mmol) in DMF (2 mL) was heated at 50° C. for 18.5 h. The reaction was filtered through an Acrodisc CR 13 mm syringe filter with a 0.45 um PTFE membrane. The reaction mixture was purified by HPLC (H₂O/ACN, 0.16% NH₄OH, XBridge 19×100) to afford the product, N-(5-(2-(2,2-dimethylpyrrolidin-1-yl)acetamido)-2-methylpyridin-3-yl)-2-(1-(2-methoxyethyl)-1H-pyrazol-4-yl)pyrazolo[5,1-b]thiazole-7-carboxamide, as a tan solid (31.3 mg). LCMS (ESI): mass calcd. for C₂₆H₃₂N₈O₃S, 536.2; m/z found, 537.2 [M+H]⁺; ¹H NMR (400 MHz, DMSO-d6) δ ppm 1.01 (s, 6H), 1.61-1.71 (m, 2H), 1.71-1.81 (m, 2H), 2.40 (s, 3H), 2.79 (t, J=1.00 Hz, 2H), 3.15 (s, 2H), 3.24 (s, 3H), 3.72 (t, J=1.00 Hz, 2H), 4.30 (t, J=1.00 Hz, 2H), 7.90 (s, 1H), 8.15 (d, J=1.00 Hz, 1H), 8.21 (s, 1H), 8.50 (s, 1H), 8.59 (s, 1H), 8.64 (d, J=1.00 Hz, 1H), 9.73 (s, 1H), 9.87 (s, 1H).

### Example 330: (R)-2-(1-(2-methoxyethyl)-1H-pyrazol-4-yl)-N-(2-methyl-5-(2-(2-methylpyrrolidin-1-yl)acetamido)pyridin-3-yl)pyrazolo[5,1-b]thiazole-7-carboxamide

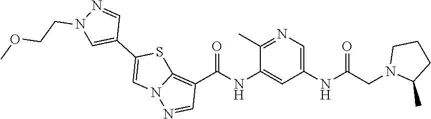 [SMILES: COCCn1cc(-c2cn3ncc(C(=O)Nc4cc(NC(=O)CN5CCC[C@H]5C)cnc4C)c3s2)cn1]

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol), (R)-(−)-2-methylpyrrolidine (12.94 mg, 0.15 mmol), and K₂CO₃ (70 mg, 0.51 mmol) in DMF (2 mL) was heated at 50° C. for 19.5 h. The reaction was filtered through an Acrodisc CR 13 mm syringe filter with a 0.45 um PTFE membrane. The reaction mixture was purified by HPLC (H₂O/ACN, 0.16% NH₄OH, XBridge 19×100) to afford the product, (R)-2-(1-(2-methoxyethyl)-1H-pyrazol-4-yl)-N-(2-methyl-5-(2-(2-methylpyrrolidin-1-yl)acetamido)pyridin-3-yl)pyrazolo[5,1-b]thiazole-7-carboxamide, as a tan solid (17.5 mg). LCMS (ESI): mass calcd. for C₂₅H₃₀N₈O₃S, 522.2; m/z found, 523.2 [M+H]⁺; ¹H NMR (400 MHz, DMSO-d6) δ ppm 1.07 (d, J=1.00 Hz, 3H), 1.29-1.46 (m, 1H), 1.61-1.83 (m, 2H), 1.84-1.98 (m, 1H), 2.33 (q, J=1.00 Hz, 1H), 2.41 (s, 3H), 2.52-2.59 (m, 1H), 3.01 (d, J=1.00 Hz, 1H), 3.11 (dt, J=1.00 Hz, 1H), 3.25 (s, 3H), 3.46 (d, J=1.00 Hz, 1H), 3.71 (t, J=1.00 Hz, 2H), 4.29 (t, J=1.00 Hz, 2H), 7.90 (s, 1H), 8.16 (d, J=1.00 Hz, 1H), 8.22 (s, 1H), 8.50 (s, 1H), 8.58 (s, 1H), 8.61 (d, J=1.00 Hz, 1H), 9.84 (s, 1H), 9.88 (s, 1H).

### Example 331: N-(5-(2-(2-azabicyclo[2.2.2]octan-2-yl)acetamido)-2-methylpyridin-3-yl)-2-(1-(2-methoxyethyl)-1H-pyrazol-4-yl)pyrazolo[5,1-b]thiazole-7-carboxamide

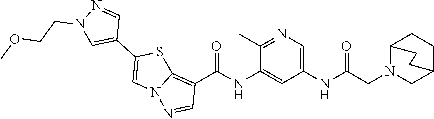 [SMILES: COCCn1cc(-c2cn3ncc(C(=O)Nc4cc(NC(=O)CN5CC6CCC5CC6)cnc4C)c3s2)cn1]

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol), 2-azabicyclo[2.2.2]octane (16.89 mg, 0.15 mmol), and K₂CO₃ (70 mg, 0.51 mmol) in DMF (2 mL) was heated at 50° C. for 19.5 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, N-(5-(2-(2-azabicyclo[2.2.2]octan-2-yl)acetamido)-2-methylpyridin-3-yl)-2-(1-(2-methoxyethyl)-1H-pyrazol-4-yl)pyrazolo[5,1-b]thiazole-7-carboxamide, as a tan solid (28.3 mg). LCMS: [M+H]⁺=549.2; [M+Na]=571.2; ¹H NMR (400 MHz, DMSO-d6) δ ppm 1.40-1.53 (m, 4H), 1.55-1.70 (m, 3H), 1.88-2.04 (m, 2H), 2.39 (s, 3H), 2.56 (s, 1H), 2.77 (s, 2H), 3.23 (s, 3H), 3.27 (s, 2H), 3.71 (t, J=1.00 Hz, 2H), 4.29 (t, J=1.00 Hz, 2H), 7.89 (s, 1H), 8.15 (d, J=1.00 Hz, 1H), 8.22 (s, 1H), 8.51 (s, 1H), 8.59 (s, 1H), 8.61 (d, J=1.00 Hz, 1H), 9.77 (s, 1H), 9.89 (s, 1H).

### Example 332: N-(5-(2-(5-azaspiro[3.4]octan-5-yl)acetamido)-2-methylpyridin-3-yl)-2-(1-(2-methoxyethyl)-1H-pyrazol-4-yl)pyrazolo[5,1-b]thiazole-7-carboxamide

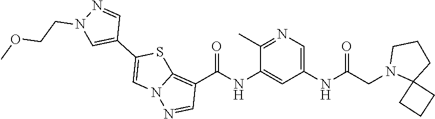 [SMILES: COCCn1cc(-c2cn3ncc(C(=O)Nc4cc(NC(=O)CN5CCCC56CCC6)cnc4C)c3s2)cn1]

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol), 5-azaspiro[3.4]octane hydrochloride (22.43 mg, 0.15 mmol), and K₂CO₃ (70 mg, 0.51 mmol) in DMF (2 mL) was heated at 50° C. for 20 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, N-(5-(2-(5-azaspiro[3.4]octan-5-yl)acetamido)-2-methylpyridin-3-yl)-2-(1-(2-methoxyethyl)-1H-pyrazol-4-yl)pyrazolo[5,1-b]thiazole-7-carboxamide, as a tan solid (39.2 mg). LCMS (ESI): mass calcd. for C₂₇H₃₂N₈O₃S, 548.2; m/z found, 549.2 [M+H]⁺; ¹H NMR (400 MHz, DMSO-d6) δ ppm 1.43-1.63 (m, 2H), 1.82-1.97 (m, 2H), 2.13-2.28 (m, 4H), 2.40 (s, 3H), 3.19 (t, J=1.00 Hz, 2H), 3.24 (s, 3H), 3.26 (s, 2H), 3.33 (s, 2H), 3.72 (t, J=1.00 Hz, 2H), 4.29 (t, J=1.00 Hz, 2H), 7.90 (s, 1H), 8.17 (d, J=1.00 Hz, 1H), 8.22 (s, 1H), 8.50 (s, 1H), 8.58 (d, J=1.00 Hz, 1H), 8.59 (s, 1H), 9.86 (s, 2H).

### Example 333: N-(5-(2-(1-azaspiro[3.3]heptan-1-yl)acetamido)-2-methylpyridin-3-yl)-2-(1-(2-methoxyethyl)-1H-pyrazol-4-yl)pyrazolo[5,1-b]thiazole-7-carboxamide

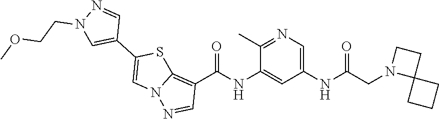 [SMILES: COCCn1cc(-c2cn3ncc(C(=O)Nc4cc(NC(=O)CN5CCC56CCC6)cnc4C)c3s2)cn1]

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol), 1-azaspiro[3.3]heptane hydrochloride (20.3 mg, 0.15 mmol), and K₂CO₃ (70 mg, 0.51 mmol) in DMF (2 mL) was heated at 50° C. for 19 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, N-(5-(2-(1-azaspiro[3.3]heptan-1-yl)acetamido)-2-methylpyridin-3-yl)-2-(1-(2-methoxyethyl)-1H-pyrazol-4-yl)pyrazolo[5,1-b]thiazole-7-carboxamide, as a tan solid (17.6 mg). LCMS (ESI): mass calcd. for C₂₆H₃₀N₈O₃S, 534.2; m/z found, 535.2 [M+H]⁺; ¹H NMR (400 MHz, DMSO-d6) δ ppm 1.53-1.64 (m, 2H), 1.64-1.78 (m, 4H), 1.95 (t, J=1.00 Hz, 2H), 2.14 (q, J=1.00 Hz, 2H), 2.41 (s, 3H), 2.73 (t, J=1.00 Hz, 2H), 3.24 (s, 3H), 3.72 (t, J=1.00 Hz, 2H), 4.30 (t, J=1.00 Hz, 2H), 7.90 (s, 1H), 8.16 (d, J=1.00 Hz, 1H), 8.21 (s, 1H), 8.50 (s, 1H), 8.58 (s, 1H), 8.61 (d, J=1.00 Hz, 1H), 9.76 (s, 1H), 9.88 (s, 1H).

### Example 334: N-(5-(2-((1R,4S)-2-azabicyclo[2.2.1]heptan-2-yl)acetamido)-2-methylpyridin-3-yl)-2-(1-(2-methoxyethyl)-1H-pyrazol-4-yl)pyrazolo[5,1-b]thiazole-7-carboxamide

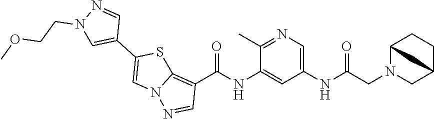 [SMILES: COCCn1cc(-c2cn3ncc(C(=O)Nc4cc(NC(=O)CN5C[C@H]6CC[C@@H]5C6)cnc4C)c3s2)cn1]

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol), 2-azabicyclo[2.2.1]heptane (14.76 mg, 0.15 mmol), and K₂CO₃ (70 mg, 0.51 mmol) in DMF (2 mL) was heated at 50° C. for 17 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, N-(5-(2-((1R,4S)-2-azabicyclo[2.2.1]heptan-2-yl)acetamido)-2-methylpyridin-3-yl)-2-(1-(2-methoxyethyl)-1H-pyrazol-4-yl)pyrazolo[5,1-b]thiazole-7-carboxamide, as a tan solid (44.2 mg). LCMS (ESI): mass calcd. for C₂₆H₃₀N₈O₃S, 534.2; m/z found, 535.2 [M+H]⁺; ¹H NMR (400 MHz, DMSO-d6) δ ppm 1.24 (d, J=1.00 Hz, 1H), 1.28-1.35 (m, 1H), 1.36-1.47 (m, 1H), 1.47-1.58 (m, 1H), 1.63-1.80 (m, 2H), 2.24 (d, J=1.00 Hz, 1H), 2.30-2.36 (m, 1H), 2.40 (s, 3H), 2.79-2.89 (m, 1H), 3.22 (s, 2H), 3.26 (s, 4H), 3.71 (t, J=1.00 Hz, 2H), 4.30 (t, J=1.00 Hz, 2H), 7.90 (s, 1H), 8.17 (d, J=1.00 Hz, 1H), 8.22 (s, 1H), 8.50 (s, 1H), 8.58 (s, 1H), 8.62 (d, J=1.00 Hz, 1H), 9.78 (s, 1H), 9.88 (s, 1H).

### Example 335: N-(5-(2-(5-azaspiro[2.5]octan-5-yl)acetamido)-2-methylpyridin-3-yl)-2-(1-(2-methoxyethyl)-1H-pyrazol-4-yl)pyrazolo[5,1-b]thiazole-7-carboxamide

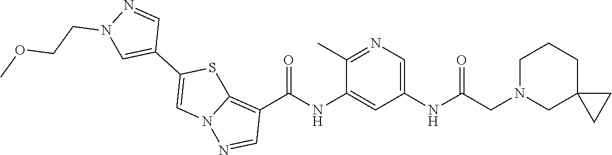 [SMILES: COCCn1cc(-c2cn3ncc(C(=O)Nc4cc(NC(=O)CN5CCCC6(CC6)C5)cnc4C)c3s2)cn1]

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol), 5-azaspiro[2.5]octane hydrochloride (16.89 mg, 0.11 mmol), and K₂CO₃ (70 mg, 0.51 mmol) in DMF (2 mL) was heated at 50° C. for 28 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, N-(5-(2-(5-azaspiro[2.5]octan-5-yl)acetamido)-2-methylpyridin-3-yl)-2-(1-(2-methoxyethyl)-1H-pyrazol-4-yl)pyrazolo[5,1-b]thiazole-7-carboxamide, as a tan solid (44.9 mg). LCMS (ESI): mass calcd. for C₂₇H₃₂N₈O₃S, 548.2; m/z found, 549.2 [M+H]⁺; ¹H NMR (400 MHz, DMSO-d6) δ ppm 0.31 (td, J=1.00 Hz, 4H), 1.27 (t, J=1.00 Hz, 2H), 1.67 (quin, J=1.00 Hz, 2H), 2.24 (s, 2H), 2.40 (s, 3H), 2.56 (t, J=1.00 Hz, 2H), 3.11 (s, 2H), 3.24 (s, 3H), 3.72 (t, J=1.00 Hz, 2H), 4.30 (t, J=1.00 Hz, 2H), 7.91 (s, 1H), 8.15 (d, J=1.00 Hz, 1H), 8.22 (s, 1H), 8.49 (s, 1H), 8.55 (d, J=1.00 Hz, 1H), 8.60 (s, 1H), 9.85 (s, 1H), 9.91 (s, 1H).

### Example 336: N-(5-(2-(3,3-dimethylpyrrolidin-1-yl)acetamido)-2-methylpyridin-3-yl)-2-(1-(2-methoxyethyl)-1H-pyrazol-4-yl)pyrazolo[5,1-b]thiazole-7-carboxamide

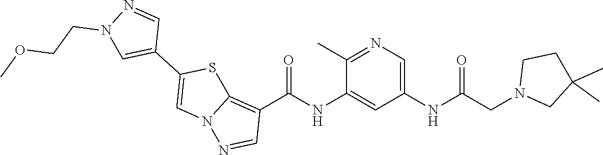

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol), 3,3-dimethylpyrrolidine hydrochloride (20.61 mg, 0.15 mmol), and K₂CO₃ (70 mg, 0.51 mmol) in DMF (2 mL) was heated at 50° C. for 19 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, N-(5-(2-(3,3-dimethylpyrrolidin-1-yl)acetamido)-2-methylpyridin-3-yl)-2-(1-(2-methoxyethyl)-1H-pyrazol-4-yl)pyrazolo[5,1-b]thiazole-7-carboxamide, as a tan solid (36.5 mg). LCMS (ESI): mass calcd. for C₂₆H₃₂N₈O₃S, 536.2; m/z found, 537.2 [M+H]⁺; ¹H NMR (400 MHz, DMSO-d6) δ ppm 1.07 (s, 6H), 1.57 (t, J=1.00 Hz, 2H), 2.39 (s, 3H), 2.43 (s, 2H), 2.73 (t, J=1.00 Hz, 2H), 3.23 (s, 3H), 3.27 (s, 2H), 3.70 (t, J=1.00 Hz, 2H), 4.29 (t, J=1.00 Hz, 2H), 7.91 (s, 1H), 8.16 (d, J=1.00 Hz, 1H), 8.22 (s, 1H), 8.50 (s, 1H), 8.56 (d, J=1.00 Hz, 1H), 8.60 (s, 1H), 9.87 (s, 2H).

### Example 337: 2-(1-(2-methoxyethyl)-1H-pyrazol-4-yl)-N-(2-methyl-5-(2-(pyrrolidin-1-yl)acetamido)pyridin-3-yl)pyrazolo[5,1-b]thiazole-7-carboxamide

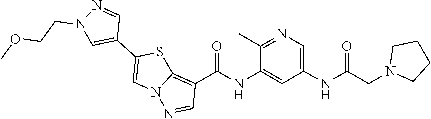 [SMILES: COCCn1cc(-c2cn3ncc(C(=O)Nc4cc(NC(=O)CN5CCCC5)cnc4C)c3s2)cn1]

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol), pyrrolidine (10.81 mg, 0.15 mmol), and K₂CO₃ (70 mg, 0.51 mmol) in DMF (2 mL) was heated at 50° C. for 4.5 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, 2-(1-(2-methoxyethyl)-1H-pyrazol-4-yl)-N-(2-methyl-5-(2-(pyrrolidin-1-yl)acetamido)pyridin-3-yl)pyrazolo[5,1-b]thiazole-7-carboxamide, as a tan solid (44.3 mg). LCMS (ESI): mass calcd. for C₂₄H₂₈N₈O₃S, 508.2; m/z found, 509.2 [M+H]⁺; ¹H NMR (400 MHz, DMSO-d6) δ ppm 1.74 (quin, J=1.00 Hz, 4H), 2.40 (s, 3H), 2.60 (quin, J=1.00 Hz, 4H), 3.24 (s, 3H), 3.28 (s, 2H), 3.72 (t, J=1.00 Hz, 2H), 4.30 (t, J=1.00 Hz, 2H), 7.90 (s, 1H), 8.17 (d, J=1.00 Hz, 1H), 8.21 (s, 1H), 8.51 (s, 1H), 8.56-8.62 (m, 2H), 9.86 (s, 1H), 9.94 (s, 1H).

### Example 338: 2-(1-(2-methoxyethyl)-1H-pyrazol-4-yl)-N-(2-methyl-5-(2-(piperidin-1-yl)acetamido)pyridin-3-yl)pyrazolo[5,1-b]thiazole-7-carboxamide

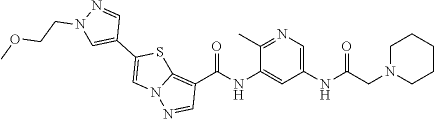 [SMILES: COCCn1cc(-c2cn3ncc(C(=O)Nc4cc(NC(=O)CN5CCCCC5)cnc4C)c3s2)cn1]

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol), piperidine (12.94 mg, 0.15 mmol), and K₂CO₃ (70 mg, 0.51 mmol) in DMF (2 mL) was heated at 50° C. for 3.5 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, 2-(1-(2-methoxyethyl)-1H-pyrazol-4-yl)-N-(2-methyl-5-(2-(piperidin-1-yl)acetamido)pyridin-3-yl)pyrazolo[5,1-b]thiazole-7-carboxamide, as a tan solid (50.4 mg). LCMS (ESI): mass calcd. for C₂₅H₃₀N₈O₃S, 522.2; m/z found, 523.2 [M+H]⁺; ¹H NMR (400 MHz, DMSO-d6) δ ppm 1.30-1.46 (m, 2H), 1.57 (quin, J=1.00 Hz, 4H), 2.38 (s, 3H), 2.43-2.49 (m, 4H), 3.09 (s, 2H), 3.25 (s, 3H), 3.73 (t, J=1.00 Hz, 2H), 4.30 (t, J=1.00 Hz, 2H), 7.89 (s, 1H), 8.15 (d, J=1.00 Hz, 1H), 8.22 (s, 1H), 8.48 (s, 1H), 8.55-8.62 (m, 2H), 9.87 (s, 2H).

### Example 339: N-(5-(2-(azepan-1-yl)acetamido)-2-methylpyridin-3-yl)-2-(1-(2-methoxyethyl)-1H-pyrazol-4-yl)pyrazolo[5,1-b]thiazole-7-carboxamide

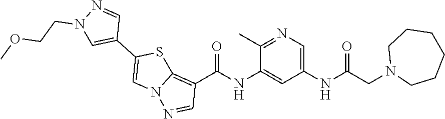 [SMILES: COCCn1cc(-c2cn3ncc(C(=O)Nc4cc(NC(=O)CN5CCCCCC5)cnc4C)c3s2)cn1]

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol), hexamethyleneimine (15 mg, 0.15 mmol), and K₂CO₃ (70 mg, 0.51 mmol) in DMF (2 mL) was heated at 50° C. for 17.5 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, N-(5-(2-(azepan-1-yl)acetamido)-2-methylpyridin-3-yl)-2-(1-(2-methoxyethyl)-1H-pyrazol-4-yl)pyrazolo[5,1-b]thiazole-7-carboxamide, as a tan solid (39.4 mg). LCMS (ESI): mass calcd. for C₂₆H₃₂N₈O₃S, 536.2; m/z found, 537.2 [M+H]⁺; ¹H NMR (400 MHz, DMSO-d6) δ ppm 1.52-1.60 (m, 4H), 1.60-1.70 (m, 4H), 2.40 (s, 3H), 2.73 (t, J=1.00 Hz, 4H), 3.24 (s, 3H), 3.28 (s, 2H), 3.71 (t, J=1.00 Hz, 2H), 4.29 (t, J=1.00 Hz, 2H), 7.90 (s, 1H), 8.17 (d, J=1.00 Hz, 1H), 8.22 (s, 1H), 8.50 (s, 1H), 8.58 (d, J=1.00 Hz, 1H), 8.59 (s, 1H), 9.85 (s, 1H), 9.88 (s, 1H).

### Example 340: N-(5-(2-(7-azabicyclo[2.2.1]heptan-7-yl)acetamido)-2-methylpyridin-3-yl)-2-(1-(2-methoxyethyl)-1H-pyrazol-4-yl)pyrazolo[5,1-b]thiazole-7-carboxamide

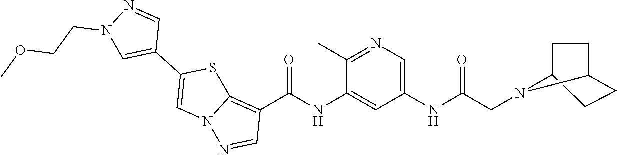 [SMILES: COCCn1cc(-c2cn3ncc(C(=O)Nc4cc(NC(=O)CN5C6CCC5CC6)cnc4C)c3s2)cn1]

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol), 7-azabicyclo[2.2.1]heptane (14.76 mg, 0.15 mmol), and K₂CO₃ (70 mg, 0.51 mmol) in DMF (2 mL) was heated at 50° C. for 2 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, N-(5-(2-(7-azabicyclo[2.2.1]heptan-7-yl)acetamido)-2-methylpyridin-3-yl)-2-(1-(2-methoxyethyl)-1H-pyrazol-4-yl)pyrazolo[5,1-b]thiazole-7-carboxamide, as a tan solid (35.0 mg). LCMS (ESI): mass calcd. for C₂₆H₃₀N₈O₃S, 534.2; m/z found, 535.2 [M+H]⁺; ¹H NMR (400 MHz, DMSO-d6) δ ppm 1.23-1.34 (m, 4H), 1.69-1.80 (m, 4H), 2.41 (s, 3H), 3.12 (s, 2H), 3.24 (s, 3H), 3.28-3.31 (m, 2H), 3.72 (t, J=1.00 Hz, 2H), 4.30 (t, J=1.00 Hz, 2H), 7.90 (s, 1H), 8.17 (d, J=1.00 Hz, 1H), 8.22 (s, 1H), 8.51 (s, 1H), 8.59 (s, 1H), 8.61 (d, J=1.00 Hz, 1H), 9.87 (s, 1H), 9.93 (s, 1H).

### Example 341: N-(5-(2-(3-cyclopropylpyrrolidin-1-yl)acetamido)-2-methylpyridin-3-yl)-2-(1-(2-methoxyethyl)-1H-pyrazol-4-yl)pyrazolo[5,1-b]thiazole-7-carboxamide

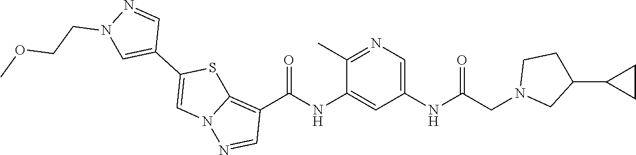 [SMILES: COCCn1cc(-c2cn3ncc(C(=O)Nc4cc(NC(=O)CN5CCC(C6CC6)C5)cnc4C)c3s2)cn1]

A mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (100 mg, 0.21 mmol), 3-cyclopropylpyrrolidine (16.89 mg, 0.15 mmol), and K₂CO₃ (116.64 mg, 0.84 mmol) in DMF (2 mL) was heated at 50° C. for 4 h. The reaction was filtered through an Acrodisc CR 13 mm syringe filter with a 0.45 mm PTFE membrane. The reaction mixture was purified by HPLC (H₂O/ACN, 0.16% NH₄OH, XBridge 19×100) to afford the product, N-(5-(2-(3-cyclopropylpyrrolidin-1-yl)acetamido)-2-methylpyridin-3-yl)-2-(1-(2-methoxyethyl)-1H-pyrazol-4-yl)pyrazolo[5,1-b]thiazole-7-carboxamide, as a tan solid (35.8 mg). LCMS (ESI): mass calcd. for C₂₇H₃₂N₈O₃S, 548.2; m/z found, 549.2 [M+H]⁺; ¹H NMR (400 MHz, DMSO-d6) δ ppm 0.05-0.12 (m, 2H), 0.32-0.43 (m, 2H), 0.72-0.85 (m, 1H), 1.48-1.65 (m, 2H), 1.87-2.02 (m, 1H), 2.37 (dd, J=1.00 Hz, 1H), 2.41 (s, 3H), 2.56-2.65 (m, 1H), 2.66-2.75 (m, 1H), 2.81 (dd, J=1.00 Hz, 1H), 3.24 (s, 3H), 3.27 (s, 1H), 3.28-3.31 (m, 1H), 3.71 (t, J=1.00 Hz, 2H), 4.29 (t, J=1.00 Hz, 2H), 7.90 (s, 1H), 8.16 (d, J=1.00 Hz, 1H), 8.22 (s, 1H), 8.50 (s, 1H), 8.57 (d, J=1.00 Hz, 1H), 8.59 (s, 1H), 9.86 (s, 1H), 9.92 (s, 1H).

### Example 342: N-(5-(1-isopropylazetidine-3-carboxamido)-2-methylpyridin-3-yl)-2-(1-(2-methoxyethyl)-1H-pyrazol-4-yl)pyrazolo[5,1-b]thiazole-7-carboxamide

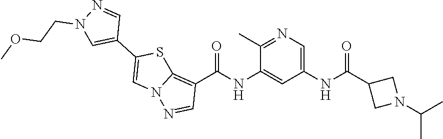 [SMILES: COCCn1cc(-c2cn3ncc(C(=O)Nc4cc(NC(=O)C5CN(C(C)C)C5)cnc4C)c3s2)cn1]

To a mixture of N-(5-amino-2-methylpyridin-3-yl)-2-(1-(2-methoxyethyl)-1H-pyrazol-4-yl)pyrazolo[5,1-b]thiazole-7-carboxamide hydrochloride (60 mg, 0.14 mmol) and 1-isopropylazetidine-3-carboxylic acid (29.70 mg, 0.21 mmol) in pyridine (1.5 mL) was added EDCI (39.76 mg, 0.21 mmol). The reaction was stirred at 25° C. for 19.5 h. All solvents were removed in vacuo. The residue was taken up in DMSO (1.5 mL) and was purified by HPLC (H₂O/ACN, 0.16% NH₄OH, XBridge 19×100) to afford the product, N-(5-(1-isopropylazetidine-3-carboxamido)-2-methylpyridin-3-yl)-2-(1-(2-methoxyethyl)-1H-pyrazol-4-yl)pyrazolo[5,1-b]thiazole-7-carboxamide, as a white solid (56.8 mg). LCMS (ESI): mass calcd. for C₂₅H₃₀N₈O₃S, 522.2; m/z found, 523.2 [M+H]⁺; ¹H NMR (400 MHz, DMSO-d6) δ ppm 0.84 (d, J=1.00 Hz, 6H), 2.24 (quin, J=1.00 Hz, 1H), 2.41 (s, 3H), 3.12 (t, J=1.00 Hz, 2H), 3.25 (s, 3H), 3.28 (br d, J=7.83 Hz, 1H), 3.38 (t, J=1.00 Hz, 2H), 3.72 (t, J=1.00 Hz, 2H), 4.30 (t, J=1.00 Hz, 2H), 7.90 (s, 1H), 8.14 (d, J=1.00 Hz, 1H), 8.22 (s, 1H), 8.51 (s, 1H), 8.54 (d, J=1.00 Hz, 1H), 8.59 (s, 1H), 9.84 (s, 1H), 10.08 (s, 1H).

### Example 343: (S)—N-(5-(1-isopropylpyrrolidine-2-carboxamido)-2-methylpyridin-3-yl)-2-(1-(2-methoxyethyl)-1H-pyrazol-4-yl)pyrazolo[5,1-b]thiazole-7-carboxamide

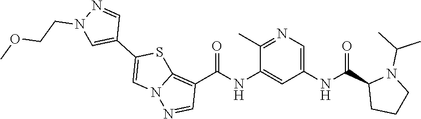 [SMILES: COCCn1cc(-c2cn3ncc(C(=O)Nc4cc(NC(=O)[C@@H]5CCCN5C(C)C)cnc4C)c3s2)cn1]

To a mixture of N-(5-amino-2-methylpyridin-3-yl)-2-(1-(2-methoxyethyl)-1H-pyrazol-4-yl)pyrazolo[5,1-b]thiazole-7-carboxamide hydrochloride (60 mg, 0.14 mmol) and N-isopropyl-L-proline (32.61 mg, 0.21 mmol) in pyridine (1.5 mL) was added EDCI (39.76 mg, 0.21 mmol). The reaction was stirred at 25° C. for 19.5 h. All solvents were removed in vacuo. The residue was taken up in DMSO (1.5 mL) and was purified by HPLC (H₂O/ACN, 0.16% NH₄OH, XBridge 19×100) to afford the product, (S)—N-(5-(1-isopropylpyrrolidine-2-carboxamido)-2-methylpyridin-3-yl)-2-(1-(2-methoxyethyl)-1H-pyrazol-4-yl)pyrazolo[5,1-b]thiazole-7-carboxamide, as an off-white solid (37.4 mg). LCMS (ESI): mass calcd. for C₂₆H₃₂N₈O₃S, 536.2; m/z found, 537.2 [M+H]⁺; ¹H NMR (400 MHz, DMSO-d6) δ ppm 1.03 (d, J=1.00 Hz, 3H), 1.07 (d, J=1.00 Hz, 3H), 1.68-1.78 (m, 2H), 1.78-1.88 (m, 1H), 1.99-2.15 (m, 1H), 2.36-2.44 (m, 3H), 2.55 (q, J=1.00 Hz, 2H), 2.79 (quin, J=1.00 Hz, 1H), 3.09-3.17 (m, 1H), 3.24 (s, 3H), 3.73 (t, J=1.00 Hz, 2H), 4.29 (t, J=1.00 Hz, 2H), 7.90 (s, 1H), 8.18 (d, J=1.00 Hz, 1H), 8.22 (s, 1H), 8.51 (s, 1H), 8.59 (s, 1H), 8.63 (d, J=1.00 Hz, 1H), 9.87 (s, 1H), 9.93 (s, 1H).

### Example 344: (R)—N-(5-(1-isopropylpyrrolidine-2-carboxamido)-2-methylpyridin-3-yl)-2-(1-(2-methoxyethyl)-1H-pyrazol-4-yl)pyrazolo[5,1-b]thiazole-7-carboxamide

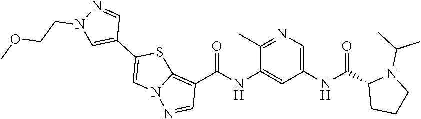 [SMILES: COCCn1cc(-c2cn3ncc(C(=O)Nc4cc(NC(=O)[C@H]5CCCN5C(C)C)cnc4C)c3s2)cn1]

To a mixture of N-(5-amino-2-methylpyridin-3-yl)-2-(1-(2-methoxyethyl)-1H-pyrazol-4-yl)pyrazolo[5,1-b]thiazole-7-carboxamide hydrochloride (60 mg, 0.14 mmol) and (2R)-1-(propan-2-yl)pyrrolidine-2-carboxylic acid (32.61 mg, 0.21 mmol) in pyridine (1.5 mL) was added EDCI (39.76 mg, 0.21 mmol). The reaction was stirred at 25° C. for 19.5 h. All solvents were removed in vacuo. The residue was taken up in DMSO (1.5 mL) and was purified by HPLC (H₂O/ACN, 0.16% NH₄OH, XBridge 19×100) to afford the product, (R)—N-(5-(1-isopropylpyrrolidine-2-carboxamido)-2-methylpyridin-3-yl)-2-(1-(2-methoxyethyl)-1H-pyrazol-4-yl)pyrazolo[5,1-b]thiazole-7-carboxamide, as an off-white solid (35.1 mg). LCMS (ESI): mass calcd. for C₂₆H₃₂N₈O₃S, 536.2; m/z found, 537.2 [M+H]⁺; ¹H NMR (400 MHz, DMSO-d6) δ ppm 1.03 (d, J=1.00 Hz, 3H), 1.07 (d, J=1.00 Hz, 3H), 1.69-1.78 (m, 2H), 1.78-1.88 (m, 1H), 1.98-2.15 (m, 1H), 2.40 (s, 3H), 2.55 (q, J=1.00 Hz, 2H), 2.80 (quin, J=1.00 Hz, 1H), 3.09-3.19 (m, 1H), 3.25 (s, 3H), 3.73 (t, J=1.00 Hz, 2H), 4.30 (t, J=1.00 Hz, 2H), 7.89 (s, 1H), 8.17 (d, J=1.00 Hz, 1H), 8.23 (s, 1H), 8.48 (s, 1H), 8.58 (s, 1H), 8.63 (d, J=1.00 Hz, 1H), 9.86 (s, 1H), 9.93 (s, 1H).

### Example 345: N-(5-(1-isopropylpyrrolidine-3-carboxamido)-2-methylpyridin-3-yl)-2-(1-(2-methoxyethyl)-1H-pyrazol-4-yl)pyrazolo[5,1-b]thiazole-7-carboxamide

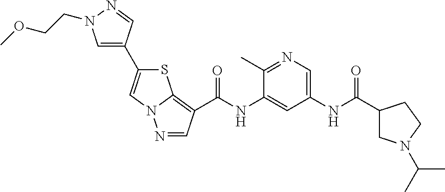 [SMILES: COCCn1cc(-c2cn3ncc(C(=O)Nc4cc(NC(=O)C5CCN(C(C)C)C5)cnc4C)c3s2)cn1]

To a mixture of N-(5-amino-2-methylpyridin-3-yl)-2-(1-(2-methoxyethyl)-1H-pyrazol-4-yl)pyrazolo[5,1-b]thiazole-7-carboxamide hydrochloride (60 mg, 0.14 mmol) and 1-(propan-2-yl)pyrrolidine-3-carboxylic acid (32.61 mg, 0.21 mmol) in pyridine (1.5 mL) was added EDCI (39.76 mg, 0.21 mmol). The reaction was stirred at 25° C. for 18 h. All solvents were removed in vacuo. The residue was dissolved in DMSO (1.5 mL) and was purified by HPLC (H₂O/ACN, 0.16% NH₄OH, XBridge 19×100) to afford the product, N-(5-(1-isopropylpyrrolidine-3-carboxamido)-2-methylpyridin-3-yl)-2-(1-(2-methoxyethyl)-1H-pyrazol-4-yl)pyrazolo[5,1-b]thiazole-7-carboxamide, as a tan solid (42.1 mg). LCMS (ESI): mass calcd. for C₂₆H₃₂N₈O₃S, 536.2; m/z found, 537.2 [M+H]⁺; ¹H NMR (400 MHz, DMSO-d6) δ ppm 1.01 (d, J=1.00 Hz, 3H), 1.05 (d, J=1.00 Hz, 3H), 1.90-2.04 (m, 2H), 2.35 (quin, J=1.00 Hz, 1H), 2.39-2.42 (m, 3H), 2.45 (q, J=1.00 Hz, 1H), 2.55 (t, J=1.00 Hz, 1H), 2.70 (q, J=1.00 Hz, 1H), 2.93 (t, J=1.00 Hz, 1H), 3.04 (quin, J=1.00 Hz, 1H), 3.25 (s, 3H), 3.71 (t, J=1.00 Hz, 2H), 4.30 (t, J=1.00 Hz, 2H), 7.91 (s, 1H), 8.13 (d, J=1.00 Hz, 1H), 8.21 (s, 1H), 8.51 (s, 1H), 8.53 (d, J=1.00 Hz, 1H), 8.59 (s, 1H), 9.83 (s, 1H), 10.10 (s, 1H).

### Example 346: (S)-2-(6,7-dihydro-5H-pyrazolo[5,1-b][1,3]oxazin-3-yl)-N-(5-(1-isopropylpyrrolidine-2-carboxamido)-2-methylpyridin-3-yl)pyrazolo[5,1-b]thiazole-7-carboxamide

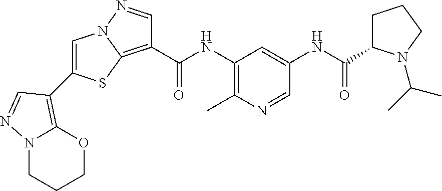 [SMILES: Cc1ncc(NC(=O)[C@@H]2CCCN2C(C)C)cc1NC(=O)c1cnn2cc(-c3cnn4c3OCCC4)sc12]

### Step a: tert-butyl (5-(2-(6,7-dihydro-5H-pyrazolo[5,1-b][1,3]oxazin-3-yl)pyrazolo[5,1-b]thiazole-7-carboxamido)-6-methylpyridin-3-yl)carbamate

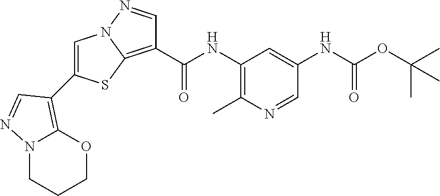 [SMILES: Cc1ncc(NC(=O)OC(C)(C)C)cc1NC(=O)c1cnn2cc(-c3cnn4c3OCCC4)sc12]

To a mixture of tert-butyl (5-(6-bromo-[1,2,3]triazolo[1,5-a]pyridine-3-carboxamido)-6-methylpyridin-3-yl)carbamate (500 mg, 1.11 mmol) and 3-(4,4,5,5-tetramethyl-1,3,2-dioxaborolan-2-yl)-6,7-dihydro-5H-pyrazolo[5,1-b][1,3]oxazine (331.76 mg, 1.33 mmol) in 1,4-dioxane (5 mL) was added a solution of K₂CO₃ (458.31 mg, 3.32 mmol) in water (1 mL) followed by the addition of 1,1′-bis(diphenylphosphino)ferrocene-palladium(II)dichloride dichlormethane complex (180.54 mg, 0.22 mmol). The reaction mixture was thoroughly flushed with argon before being capped and heated at 100° C. for 19 h. The reaction was diluted with MeOH (25 mL) and silica gel (5 g) was added. All solvents were removed in vacuo. The silica gel mesh was loaded on a Redi Sep Rf silica gel cartridge (40 g) eluting with MeOH/CH₂Cl₂ (0-30%) over 15 min. to afford a mixture of products. The solid was taken up in 20% MeOH/CH₂Cl₂ (25 mL) and silica gel (3 g). All solvents were removed in vacuo. The silica gel mesh was loaded on a Redi Sep Rf silicaa gel cartridge (40 g) eluting with EtOAc over 5 min then MeOH/EtOAc (0-30%) over 15 min to afford the product, tert-butyl (5-(2-(6,7-dihydro-5H-pyrazolo[5,1-b][1,3]oxazin-3-yl)pyrazolo[5,1-b]thiazole-7-carboxamido)-6-methylpyridin-3-yl)carbamate, as a tan solid (229.4 mg). LCMS (ESI): mass calcd. for C₂₃H₂₅N₇O₄S, 495.2; m/z found, 496.1 [M+H]⁺; ¹H NMR (400 MHz, DMSO-d6) δ ppm 1.48 (s, 9H), 2.24 (quin, J=1.00 Hz, 2H), 2.36 (s, 3H), 4.13 (t, J=1.00 Hz, 2H), 4.49 (t, J=1.00 Hz, 2H), 7.76 (s, 1H), 7.98 (br s, 1H), 8.33 (s, 1H), 8.36 (d, J=1.00 Hz, 1H), 8.46 (br s, 1H), 9.55 (br s, 1H), 9.81 (s, 1H).

### Step b: N-(5-amino-2-methylpyridin-3-yl)-2-(6,7-dihydro-5H-pyrazolo[5,1-b][1,3]oxazin-3-yl)pyrazolo[5,1-b]thiazole-7-carboxamide

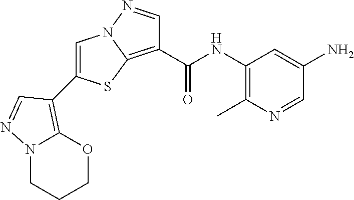 [SMILES: Cc1ncc(N)cc1NC(=O)c1cnn2cc(-c3cnn4c3OCCC4)sc12]

To a solution of tert-butyl (5-(2-(6,7-dihydro-5H-pyrazolo[5,1-b][1,3]oxazin-3-yl)pyrazolo[5,1-b]thiazole-7-carboxamido)-6-methylpyridin-3-yl)carbamate (730 mg, 1.47 mmol) in DCM (20 mL) was added HCl (4M in dioxane) (18.4 mL, 4 M, 73.7 mmol). The reaction was capped and stirred at 25° C. for 4.5 h. All solvents were removed in vacuo. The solid was dried under high vacuum to afford the HCl salt of the product, N-(5-amino-2-methylpyridin-3-yl)-2-(6,7-dihydro-5H-pyrazolo[5,1-b][1,3]oxazin-3-yl)pyrazolo[5,1-b]thiazole-7-carboxamide, as a tan solid (quantitative yield). The product was used without any further purification. LCMS (ESI): mass calcd. for C₁₈H₁₇N₇O₂S, 395.1; m/z found, 396.1 [M+H]⁺; ¹H NMR (400 MHz, DMSO-d6) δ ppm 2.24 (quin, J=1.00 Hz, 2H), 3.57 (s, 3H), 4.13 (t, J=1.00 Hz, 2H), 4.49 (t, J=1.00 Hz, 2H), 6.89 (br s, 3H), 7.72-7.80 (m, 2H), 7.83 (d, J=1.00 Hz, 1H), 8.37 (s, 1H), 8.64 (s, 1H), 10.34 (s, 1H).

### Step c: (S)-2-(6,7-dihydro-5H-pyrazolo[5,1-b][1,3]oxazin-3-yl)-N-(5-(1-isopropylpyrrolidine-2-carboxamido)-2-methylpyridin-3-yl)pyrazolo[5,1-b]thiazole-7-carboxamide

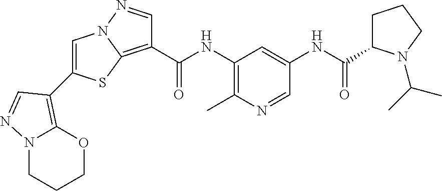 [SMILES: Cc1ncc(NC(=O)[C@@H]2CCCN2C(C)C)cc1NC(=O)c1cnn2cc(-c3cnn4c3OCCC4)sc12]

To a solution of N-(5-amino-2-methylpyridin-3-yl)-2-(6,7-dihydro-5H-pyrazolo[5,1-b][1,3]oxazin-3-yl)pyrazolo[5,1-b]thiazole-7-carboxamide hydrochloride (60 mg, 0.14 mmol) and N-isopropyl-L-proline (32.76 mg, 0.21 mmol) in pyridine (1.5 mL) was added EDCI (39.95 mg, 0.21 mmol). The reaction was stirred at 25° C. for 20 h. All solvents were removed in vacuo. The residue was taken up in DMSO (1.5 mL) and was purified by HPLC (H₂O/ACN, 0.16% NH₄OH, XBridge 19×100) to afford the product, (S)-2-(6,7-dihydro-5H-pyrazolo[5,1-b][1,3]oxazin-3-yl)-N-(5-(1-isopropylpyrrolidine-2-carboxamido)-2-methylpyridin-3-yl)pyrazolo[5,1-b]thiazole-7-carboxamide, as a tan solid (23.3 mg). LCMS (ESI): mass calcd. for C₂₆H₃₀N₈O₃S, 534.2; m/z found, 535.2 [M+H]⁺; ¹H NMR (400 MHz, DMSO-d6) δ ppm 1.03 (d, J=1.00 Hz, 3H), 1.08 (d, J=1.00 Hz, 3H), 1.68-1.79 (m, 2H), 1.79-1.88 (m, 1H), 2.07 (quin, J=1.00 Hz, 1H), 2.25 (quin, J=1.00 Hz, 2H), 2.40 (s, 3H), 2.55 (q, J=1.00 Hz, 1H), 2.79 (quin, J=1.00 Hz, 1H), 3.07-3.18 (m, 1H), 3.26-3.31 (m, 1H), 4.12 (t, J=1.00 Hz, 2H), 4.48 (t, J=1.00 Hz, 2H), 7.77 (s, 1H), 8.20 (d, J=1.00 Hz, 1H), 8.35 (s, 1H), 8.47 (s, 1H), 8.61 (d, J=1.00 Hz, 1H), 9.83 (s, 1H), 9.93 (s, 1H).

### Example 347: (R)-2-(6,7-dihydro-5H-pyrazolo[5,1-b][1,3]oxazin-3-yl)-N-(5-(1-isopropylpyrrolidine-2-carboxamido)-2-methylpyridin-3-yl)pyrazolo[5,1-b]thiazole-7-carboxamide

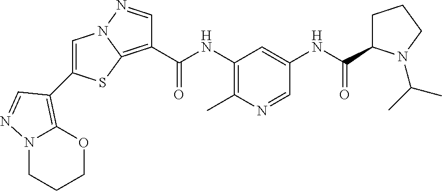 [SMILES: Cc1ncc(NC(=O)[C@H]2CCCN2C(C)C)cc1NC(=O)c1cnn2cc(-c3cnn4c3OCCC4)sc12]

To a solution of N-(5-amino-2-methylpyridin-3-yl)-2-(6,7-dihydro-5H-pyrazolo[5,1-b][1,3]oxazin-3-yl)pyrazolo[5,1-b]thiazole-7-carboxamide hydrochloride (60 mg, 0.14 mmol) and (2R)-1-(propan-2-yl)pyrrolidine-2-carboxylic acid (32.76 mg, 0.21 mmol) in pyridine (1.5 mL) was added EDCI (39.95 mg, 0.21 mmol). The reaction was stirred at 25° C. for 18.5 h. All solvents were removed in vacuo. The residue was taken up in DMSO (1.5 mL) and submitted to the purification group for isolation to afford the product, (R)-2-(6,7-dihydro-5H-pyrazolo[5,1-b][1,3]oxazin-3-yl)-N-(5-(1-isopropylpyrrolidine-2-carboxamido)-2-methylpyridin-3-yl)pyrazolo[5,1-b]thiazole-7-carboxamide, as a tan solid (21.0 mg). LCMS (ESI): mass calcd. for C₂₆H₃₀N₈ (3S, 534.2; m/z found, 535.2 [M+H]⁺; ¹H NMR (400 MHz, DMSO-d6) δ ppm 1.03 (d, J=1.00 Hz, 3H), 1.07 (d, J=1.00 Hz, 3H), 1.67-1.79 (m, 2H), 1.79-1.89 (m, 1H), 2.07 (quin, J=1.00 Hz, 1H), 2.25 (quin, J=1.00 Hz, 2H), 2.40 (s, 3H), 2.56 (q, J=1.00 Hz, 1H), 2.79 (quin, J=1.00 Hz, 1H), 3.07-3.19 (m, 1H), 3.26-3.31 (m, 1H), 4.14 (t, J=1.00 Hz, 2H), 4.48 (t, J=1.00 Hz, 2H), 7.78 (s, 1H), 8.19 (d, J=1.00 Hz, 1H), 8.34 (s, 1H), 8.48 (s, 1H), 8.61 (d, J=1.00 Hz, 1H), 9.83 (s, 1H), 9.93 (s, 1H).

### Example 348: 2-(6,7-dihydro-5H-pyrazolo[5,1-b][1,3]oxazin-3-yl)-N-(5-(1-isopropylazetidine-3-carboxamido)-2-methylpyridin-3-yl)pyrazolo[5,1-b]thiazole-7-carboxamide

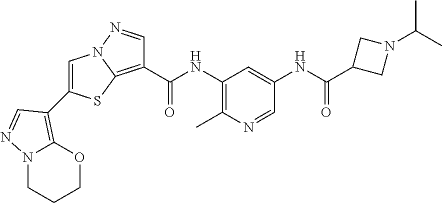 [SMILES: Cc1ncc(NC(=O)C2CN(C(C)C)C2)cc1NC(=O)c1cnn2cc(-c3cnn4c3OCCC4)sc12]

To a solution of N-(5-amino-2-methylpyridin-3-yl)-2-(6,7-dihydro-5H-pyrazolo[5,1-b][1,3]oxazin-3-yl)pyrazolo[5,1-b]thiazole-7-carboxamide hydrochloride (60 mg, 0.14 mmol) and 1-isopropylazetidine-3-carboxylic acid (29.84 mg, 0.21 mmol)) in pyridine (1.5 mL) was added EDCI (39.95 mg, 0.21 mmol). The reaction was stirred at 25° C. for 17.5 h. All solvents were removed in vacuo. The residue was taken up in DMSO (1.5 mL) and submitted to the purification group for isolation to afford the product, 2-(6,7-dihydro-5H-pyrazolo[5,1-b][1,3]oxazin-3-yl)-N-(5-(1-isopropylazetidine-3-carboxamido)-2-methylpyridin-3-yl)pyrazolo[5,1-b]thiazole-7-carboxamide, as a tan solid (37.8 mg). LCMS (ESI): mass calcd. for C₂₅H₂₈N₈O₃S, 520.2; m/z found, 521.2 [M+H]⁺; ¹H NMR (400 MHz, DMSO-d6) δ ppm 0.83 (d, J=1.00 Hz, 6H), 2.23 (quin, J=1.00 Hz, 3H), 2.39 (s, 3H), 3.10 (t, J=1.00 Hz, 2H), 3.26 (quin, J=1.00 Hz, 1H), 3.38 (t, J=1.00 Hz, 2H), 4.13 (t, J=1.00 Hz, 2H), 4.49 (t, J=1.00 Hz, 2H), 7.75 (s, 1H), 8.16 (d, J=1.00 Hz, 1H), 8.34 (s, 1H), 8.47 (s, 1H), 8.52 (d, J=1.00 Hz, 1H), 9.81 (s, 1H), 10.09 (s, 1H).

### Example 349: (S)-2-(6,7-dihydro-5H-pyrazolo[5,1-b][1,3]oxazin-3-yl)-N-(2-methyl-5-(1-methylpyrrolidine-2-carboxamido)pyridin-3-yl)pyrazolo[5,1-b]thiazole-7-carboxamide

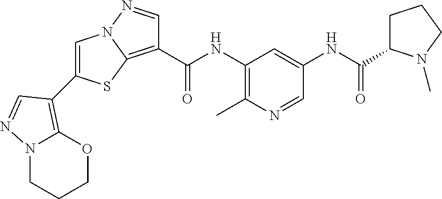 [SMILES: Cc1ncc(NC(=O)[C@@H]2CCCN2C)cc1NC(=O)c1cnn2cc(-c3cnn4c3OCCC4)sc12]

To a mixture of N-(5-amino-2-methylpyridin-3-yl)-2-(6,7-dihydro-5H-pyrazolo[5,1-b][1,3]oxazin-3-yl)pyrazolo[5,1-b]thiazole-7-carboxamide hydrochloride (60 mg, 0.14 mmol) and methyl-D-proline (26.91 mg, 0.21 mmol) in pyridine (1.5 mL, 0.982 g/mL, 18.6 mmol) was added EDCI (39.95 mg, 0.21 mmol). The reaction was stirred at 25° C. for 20 h. All solvents were removed in vacuo. The residue was taken up in DMSO (1.5 mL) and was purified by HPLC (H₂O/ACN, 0.16% NH₄OH, XBridge 19×100) to afford the product, (S)-2-(6,7-dihydro-5H-pyrazolo[5,1-b][1,3]oxazin-3-yl)-N-(2-methyl-5-(1-methylpyrrolidine-2-carboxamido)pyridin-3-yl)pyrazolo[5,1-b]thiazole-7-carboxamide, as a white solid (40.0 mg). LCMS (ESI): mass calcd. for C₂₄H₂₆N₈O₃S, 506.2; m/z found, 507.1 [M+H]⁺; ¹H NMR (400 MHz, DMSO-d6) δ ppm 1.69-1.91 (m, 3H), 2.10-2.21 (m, 1H), 2.25 (quin, J=1.00 Hz, 2H), 2.30-2.34 (m, 1H), 2.36 (s, 3H), 2.41 (s, 3H), 2.86-3.03 (m, 1H), 3.05-3.18 (m, 1H), 4.13 (t, J=1.00 Hz, 2H), 4.48 (t, J=1.00 Hz, 2H), 7.78 (s, 1H), 8.22 (d, J=1.00 Hz, 1H), 8.33 (s, 1H), 8.47 (s, 1H), 8.61 (d, J=1.00 Hz, 1H), 9.80 (s, 1H), 9.92 (br s, 1H).

### Example 350: 2-(6,7-dihydro-5H-pyrazolo[5,1-b][1,3]oxazin-3-yl)-N-(5-(1-isopropylpyrrolidine-3-carboxamido)-2-methylpyridin-3-yl)pyrazolo[5,1-b]thiazole-7-carboxamide

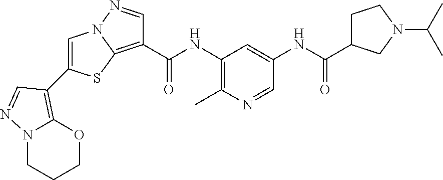 [SMILES: Cc1ncc(NC(=O)C2CCN(C(C)C)C2)cc1NC(=O)c1cnn2cc(-c3cnn4c3OCCC4)sc12]

To a mixture of N-(5-amino-2-methylpyridin-3-yl)-2-(1-(2-methoxyethyl)-1H-pyrazol-4-yl)pyrazolo[5,1-b]thiazole-7-carboxamide hydrochloride (60 mg, 0.14 mmol) and 1-(propan-2-yl)pyrrolidine-3-carboxylic acid (32.76 mg, 0.21 mmol) in pyridine (1.5 mL) was added EDCI (39.95 mg, 0.21 mmol). The reaction was stirred at 25° C. for 18 h. All solvents were removed in vacuo. The residue was dissolved in DMSO (1.5 mL) and was purified by HPLC (H₂O/ACN, 0.16% NH₄OH, XBridge 19×100) to afford the product, 2-(6,7-dihydro-5H-pyrazolo[5,1-b][1,3]oxazin-3-yl)-N-(5-(1-isopropylpyrrolidine-3-carboxamido)-2-methylpyridin-3-yl)pyrazolo[5,1-b]thiazole-7-carboxamide, as a tan solid (40.5 mg). LCMS (ESI): mass calcd. for C₂₆H₃₀N₈O₃S, 534.2; m/z found, 535.2 [M+H]⁺; ¹H NMR (400 MHz, DMSO-d6) δ ppm 1.02 (d, J=1.00 Hz, 3H), 1.04 (d, J=1.00 Hz, 3H), 1.88-2.04 (m, 2H), 2.25 (quin, J=1.00 Hz, 2H), 2.35 (quin, J=1.00 Hz, 1H), 2.41 (s, 3H), 2.46 (q, J=1.00 Hz, 1H), 2.54 (t, J=1.00 Hz, 1H), 2.70 (q, J=1.00 Hz, 1H), 2.94 (t, J=1.00 Hz, 1H), 3.03 (quin, J=1.00 Hz, 1H), 4.14 (t, J=1.00 Hz, 2H), 4.49 (t, J=1.00 Hz, 2H), 7.76 (s, 1H), 8.15 (d, J=1.00 Hz, 1H), 8.34 (s, 1H), 8.48 (s, 1H), 8.52 (d, J=1.00 Hz, 1H), 9.80 (s, 1H), 10.09 (s, 1H).

### Example 351: (R)-2-(6,7-dihydro-5H-pyrazolo[5,1-b][1,3]oxazin-3-yl)-N-(5-(2-(1-isopropylpyrrolidin-2-yl)acetamido)-2-methylpyridin-3-yl)pyrazolo[5,1-b]thiazole-7-carboxamide

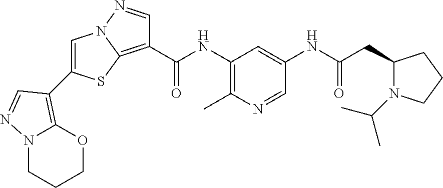 [SMILES: Cc1ncc(NC(=O)C[C@H]2CCCN2C(C)C)cc1NC(=O)c1cnn2cc(-c3cnn4c3OCCC4)sc12]

To a solution of N-(5-amino-2-methylpyridin-3-yl)-2-(6,7-dihydro-5H-pyrazolo[5,1-b][1,3]oxazin-3-yl)pyrazolo[5,1-b]thiazole-7-carboxamide hydrochloride (60 mg, 0.14 mmol) and lithium (R)-2-(1-isopropylpyrrolidin-2-yl)acetate (57.58 mg, 0.33 mmol) in pyridine (1.5 mL) was added EDCI (39.95 mg, 0.21 mmol). The reaction was stirred at 25° C. for 18 h. All solvents were removed in vacuo. The residue was dissolved in DMSO (1.5 mL) and was purified by HPLC (H₂O/ACN, 0.16% NH₄OH, XBridge 19×100) to afford the product, (R)-2-(6,7-dihydro-5H-pyrazolo[5,1-b][1,3]oxazin-3-yl)-N-(5-(2-(1-isopropylpyrrolidin-2-yl)acetamido)-2-methylpyridin-3-yl)pyrazolo[5,1-b]thiazole-7-carboxamide, as a tan solid (8.8 mg). LCMS (ESI): mass calcd. for C₂₇H₃₂N₈O₃S, 548.2; m/z found, 549.2 [M+H]⁺; ¹H NMR (400 MHz, DMSO-d6) δ ppm 0.95 (d, J=1.00 Hz, 3H), 1.07 (d, J=1.00 Hz, 3H), 1.48-1.60 (m, 1H), 1.60-1.73 (m, 2H), 1.76-1.90 (m, 1H), 2.20-2.33 (m, 3H), 2.39 (s, 3H), 2.43-2.48 (m, 1H), 2.52-2.56 (m, 1H), 2.73-2.82 (m, 1H), 2.95 (quin, J=1.00 Hz, 1H), 3.09-3.21 (m, 1H), 4.13 (t, J=1.00 Hz, 2H), 4.49 (t, J=1.00 Hz, 2H), 7.77 (s, 1H), 8.13 (d, J=1.00 Hz, 1H), 8.34 (s, 1H), 8.45-8.49 (m, 2H), 9.82 (s, 1H), 10.30 (s, 1H).

### Example 352: (S)—N-(2-methyl-5-(2-(2-methylpyrrolidin-1-yl)acetamido)pyridin-3-yl)-2-(2-morpholinopyridin-4-yl)pyrazolo[5,1-b]thiazole-7-carboxamide

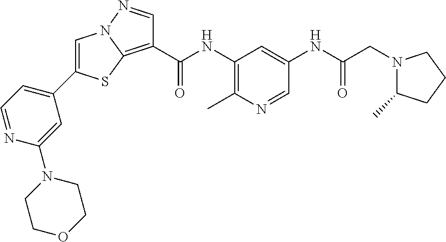 [SMILES: Cc1ncc(NC(=O)CN2CCC[C@@H]2C)cc1NC(=O)c1cnn2cc(-c3ccnc(N4CCOCC4)c3)sc12]

### Step a: tert-butyl (6-methyl-5-(2-(2-morpholinopyridin-4-yl)pyrazolo[5,1-b]thiazole-7-carboxamido)pyridin-3-yl)carbamate

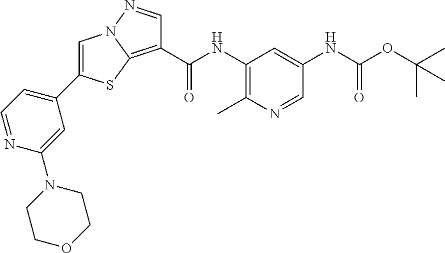 [SMILES: Cc1ncc(NC(=O)OC(C)(C)C)cc1NC(=O)c1cnn2cc(-c3ccnc(N4CCOCC4)c3)sc12]

To a mixture of tert-butyl (5-(6-bromo-[1,2,3]triazolo[1,5-a]pyridine-3-carboxamido)-6-methylpyridin-3-yl)carbamate (0.7 g, 1.548 mmol) and 2-morpholinopyridine-4-boronic acid, pinacol ester (538.86 mg, 1.86 mmol) in 1,4-dioxane (7.5 mL) was added a solution of K₂CO₃ (641.64 mg, 4.64 mmol) in water (1.5 mL) followed by the addition of 1,1′-bis(diphenylphosphino)ferrocene-palladium(II)dichloride dichlormethane complex (252.76 mg, 0.31 mmol). The reaction was thoroughly flushed with argon before being capped and heated at 100° C. for 18.5 h. LCMS indicated the starting bromide had been consumed. Silica gel (5 g) was added to the reaction and all solvents were removed in vacuo. This silica gel mesh was loaded on a Redi Sep Rf silica gel cartridge eluting with CH₂Cl₂ over 5 min then MeOH/CH₂Cl₂ (0-30%) over 15 min to afford dark solid. The solid was taken up in 20% MeOH/CH₂Cl₂ (25 mL and silica gel (5 g). All solvents were removed in vacuo. The silica gel mesh was loaded on a Redi Sep Rf silica gel cartridge eluting with EtOAc over 5 min then MeOH/EtOAc (0-30%) over 15 min to afford the product, tert-butyl (6-methyl-5-(2-(2-morpholinopyridin-4-yl)pyrazolo[5,1-b]thiazole-7-carboxamido)pyridin-3-yl)carbamate, as a tan solid (412.4 mg). LCMS (ESI): mass calcd. for C₂₆H₂₉N₇O₄S, 535.2; m/z found, 536.2 [M+H]⁺; ¹H NMR (400 MHz, DMSO-d6) δ ppm 1.48 (s, 9H), 2.37 (s, 3H), 3.55 (t, J=1.00 Hz, 4H), 3.71 (t, J=1.00 Hz, 4H), 6.93-7.07 (m, 1H), 7.13 (s, 1H), 7.99 (br s, 1H), 8.21 (d, J=1.00 Hz, 1H), 8.37 (d, J=1.00 Hz, 1H), 8.60 (s, 1H), 9.21 (s, 1H), 9.56 (br s, 1H), 9.92 (s, 1H).

### Step b: N-(5-amino-2-methylpyridin-3-yl)-2-(2-morpholinopyridin-4-yl)pyrazolo[5,1-b]thiazole-7-carboxamide

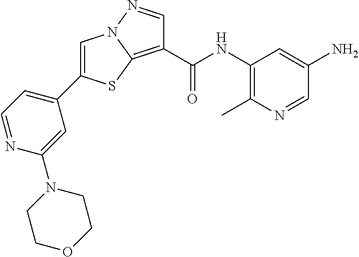 [SMILES: Cc1ncc(N)cc1NC(=O)c1cnn2cc(-c3ccnc(N4CCOCC4)c3)sc12]

To a solution of tert-butyl (6-methyl-5-(2-(2-morpholinopyridin-4-yl)pyrazolo[5,1-b]thiazole-7-carboxamido)pyridin-3-yl)carbamate (410 mg, 0.77 mmol) in DCM (10 mL) was added HCl (4M in dioxane) (4.8 mL, 4 M, 19.1 mmol). The reaction was stirred at 25° C. for 3.5 h. All solvents were removed in vacuo. The residue was dried under high vacuum to afford the HCl salt of the product, N-(5-amino-2-methylpyridin-3-yl)-2-(2-morpholinopyridin-4-yl)pyrazolo[5,1-b]thiazole-7-carboxamide, as a yellow solid (quantitative yield). The product was used without further purification. LCMS (ESI): mass calcd. for C₂₁H₂₁N₇O₂S, 435.1; m/z found, 436.1 [M+H]⁺; ¹H NMR (400 MHz, DMSO-d6) δ ppm 2.54 (s, 3H), 3.64-3.73 (m, 4H), 3.73-3.82 (m, 4H), 6.22 (br s, 3H), 7.21 (d, J=1.00 Hz, 1H), 7.37 (s, 1H), 7.80 (dd, J=1.00 Hz, 2H), 8.16 (d, J=1.00 Hz, 1H), 8.85 (s, 1H), 9.42 (s, 1H), 10.58 (s, 1H).

### Step c: N-(5-(2-chloroacetamido)-2-methylpyridin-3-yl)-2-(2-morpholinopyridin-4-yl)pyrazolo[5,1-b]thiazole-7-carboxamide

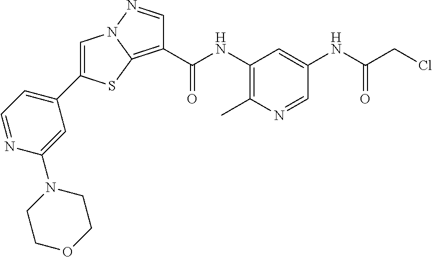 [SMILES: Cc1ncc(NC(=O)CCl)cc1NC(=O)c1cnn2cc(-c3ccnc(N4CCOCC4)c3)sc12]

To a solution of N-(5-amino-2-methylpyridin-3-yl)-2-(2-morpholinopyridin-4-yl)pyrazolo[5,1-b]thiazole-7-carboxamide hydrochloride (360 mg, 0.76 mmol) and Et₃N (0.32 mL, 0.728 g/mL, 2.29 mmol) in DCM (20 mL) was added chloroacetyl chloride (0.073 mL, 1.42 g/mL, 0.92 mmol) drop wise over a minute. The reaction was stirred at 25° C. for 16 h. Silica gel (5 g) was added to the reaction and all solvents were removed in vacuo. The silica gel mesh was loaded on a Redi Sep Rf silica gel cartridge (40 g) eluting with EtOAc over 5 min then MeOH/EtOAc (0-30%) over 15 min to afford the product, N-(5-(2-chloroacetamido)-2-methylpyridin-3-yl)-2-(2-morpholinopyridin-4-yl)pyrazolo[5,1-b]thiazole-7-carboxamide, as a tan solid (325.2 mg). LCMS (ESI): mass calcd. for C₂₃H₂₂ClN₇O₃S, 511.1; m/z found, 512.1 [M+H]⁺; ¹H NMR (400 MHz, DMSO-d6) δ ppm 2.42 (s, 3H), 3.55 (t, J=1.00 Hz, 4H), 3.73 (t, J=1.00 Hz, 4H), 4.30 (s, 2H), 7.01 (dd, J=1.00 Hz, 1H), 7.13 (s, 1H), 8.15 (d, J=1.00 Hz, 1H), 8.22 (d, J=1.00 Hz, 1H), 8.51 (d, J=1.00 Hz, 1H), 8.64 (s, 1H), 9.21 (s, 1H), 9.96 (s, 1H), 10.54 (s, 1H).

### Step d: (S)—N-(2-methyl-5-(2-(2-methylpyrrolidin-1-yl)acetamido)pyridin-3-yl)-2-(2-morpholinopyridin-4-yl)pyrazolo[5,1-b]thiazole-7-carboxamide

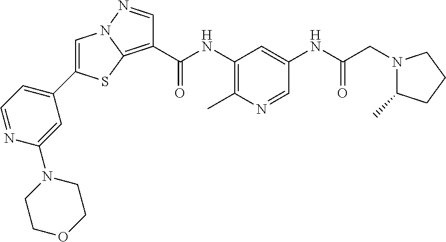 [SMILES: Cc1ncc(NC(=O)CN2CCC[C@@H]2C)cc1NC(=O)c1cnn2cc(-c3ccnc(N4CCOCC4)c3)sc12]

To a solution of N-(5-(2-chloroacetamido)-2-methylpyridin-3-yl)-2-(2-morpholinopyridin-4-yl)pyrazolo[5,1-b]thiazole-7-carboxamide (60 mg, 0.12 mmol) and (S)-(+)-methylpyrrolidine (11.97 mg, 0.14 mmol) in DMF (2 mL) was added K₂CO₃ (64.79 mg, 0.47 mmol). The reaction was heated at 50° C. for 1.5 h. The reaction was filtered through an Acrodisc CR 13 mm syringe filter with a 0.45 mm PTFE membrane. The reaction mixture was was purified by HPLC (H₂O/ACN, 0.16% NH₄OH, XBridge 19×100) to afford the product, (S)—N-(2-methyl-5-(2-(2-methylpyrrolidin-1-yl)acetamido)pyridin-3-yl)-2-(2-morpholinopyridin-4-yl)pyrazolo[5,1-b]thiazole-7-carboxamide, as a tan solid (48.5 mg). LCMS (ESI): mass calcd. for C₂₈H₃₂N₈O₃S, 560.2; m/z found, 561.2 [M+H]⁺; ¹H NMR (400 MHz, DMSO-d6) δ ppm 1.09 (d, J=1.00 Hz, 3H), 1.31-1.47 (m, 1H), 1.60-1.71 (m, 1H), 1.71-1.82 (m, 1H), 1.86-1.99 (m, 1H), 2.33 (q, J=1.00 Hz, 1H), 2.42 (s, 3H), 2.52-2.59 (m, 1H), 3.02 (d, J=1.00 Hz, 1H), 3.12 (dt, J=1.00 Hz, 1H), 3.46 (d, J=1.00 Hz, 1H), 3.55 (t, J=1.00 Hz, 4H), 3.72 (t, J=1.00 Hz, 4H), 7.01 (dd, J=1.00 Hz, 1H), 7.13 (s, 1H), 8.18 (d, J=1.00 Hz, 1H), 8.21 (d, J=1.00 Hz, 1H), 8.59 (d, J=1.00 Hz, 1H), 8.62 (s, 1H), 9.19 (s, 1H), 9.83 (s, 1H), 9.97 (s, 1H).

### Example 353: N-(5-(2-(1-azaspiro[3.3]heptan-1-yl)acetamido)-2-methylpyridin-3-yl)-2-(2-morpholinopyridin-4-yl)pyrazolo[5,1-b]thiazole-7-carboxamide

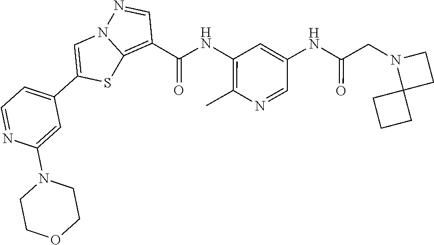 [SMILES: Cc1ncc(NC(=O)CN2CCC23CCC3)cc1NC(=O)c1cnn2cc(-c3ccnc(N4CCOCC4)c3)sc12]

A mixture of N-(5-(2-chloroacetamido)-2-methylpyridin-3-yl)-2-(2-morpholinopyridin-4-yl)pyrazolo[5,1-b]thiazole-7-carboxamide (60 mg, 0.12 mmol), 1-azaspiro[3.3]heptane hydrochloride (18.79 mg, 0.14 mmol), and K₂CO₃ (48.59 mg, 0.35 mmol) in DMF (2 mL) was heated at 50° C. for 3 h. The reaction was filtered through an Acrodisc CR 13 mm syringe filter with a 0.45 mm PTFE membrane. The reaction mixture was was purified by HPLC (H₂O/ACN, 0.16% NH₄OH, XBridge 19×100) to afford the product, N-(5-(2-(1-azaspiro[3.3]heptan-1-yl)acetamido)-2-methylpyridin-3-yl)-2-(2-morpholinopyridin-4-yl)pyrazolo[5,1-b]thiazole-7-carboxamide, as a tan solid (17.2 mg). LCMS (ESI): mass calcd. for C₂₉H₃₂N₈O₃S, 572.2; m/z found, 573.2 [M+H]⁺; ¹H NMR (400 MHz, DMSO-d6) δ ppm 1.48-1.60 (m, 2H), 1.84-1.96 (m, 2H), 2.12-2.26 (m, 4H), 2.40 (s, 3H), 3.17 (t, J=1.00 Hz, 2H), 3.25 (s, 2H), 3.55 (t, J=1.00 Hz, 4H), 3.72 (t, J=1.00 Hz, 4H), 7.01 (d, J=1.00 Hz, 1H), 7.13 (s, 1H) 8.19 (d, J=1.00 Hz, 1H), 8.21 (d, J=1.00 Hz, 1H), 8.57 (d, J=1.00 Hz, 1H), 8.61 (s, 1H) 9.19 (s, 1H), 9.86 (s, 1H), 9.97 (s, 1H).

### Example 354: 2-(1,3-dimethyl-1H-pyrazol-4-yl)-N-(5-(1-isopropylazetidine-3-carboxamido)-2-methylpyridin-3-yl)pyrazolo[5,1-b]thiazole-7-carboxamide

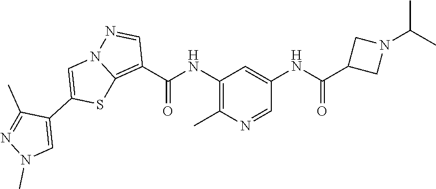 [SMILES: Cc1ncc(NC(=O)C2CN(C(C)C)C2)cc1NC(=O)c1cnn2cc(-c3cn(C)nc3C)sc12]

### Step a: tert-butyl (5-(2-(1,3-dimethyl-1H-pyrazol-4-yl)pyrazolo[5,1-b]thiazole-7-carboxamido)-6-methylpyridin-3-yl)carbamate

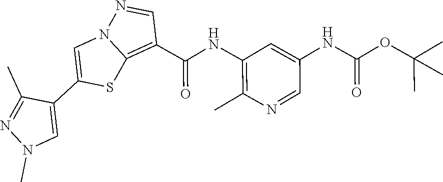 [SMILES: Cc1ncc(NC(=O)OC(C)(C)C)cc1NC(=O)c1cnn2cc(-c3cn(C)nc3C)sc12]

To a mixture of tert-butyl (5-(6-bromo-[1,2,3]triazolo[1,5-a]pyridine-3-carboxamido)-6-methylpyridin-3-yl)carbamate (0.85 g, 1.88 mmol) and 1,3-dimethyl-4-(4,4,5,5-tetramethyl-1,3,2-dioxaborolan-2-yl)-1H-pyrazole (500.82 mg, 2.26 mmol) in 1,4-dioxane (10 mL) was added a solution of K₂CO₃ (779 mg, 5.64 mmol) in water (2 mL). The reaction mixture was thoroughly flushed with argon before being capped and heated at 100° C. for 16 h. LCMS indicated the starting bromide had been consumed. The reaction was diluted with MeOH (25 mL) and silica gel (5 g) was added. All solvents were removed in vacuo. The silica gel mesh was loaded on a Redi Sep Rf silica gel cartridge (40 g) eluting with MeOH/CH₂Cl₂ (0-30%) over 15 min. to afford brown product. The solid was taken up in 20% MeOH/CH₂Cl₂ (25 mL) and silica gel (3 g). All solvents were removed in vacuo. The silica gel mesh was loaded on a Redi Sep Rf silica gel cartridge (40 g) eluting with EtOAc over 5 min then MeOH/EtOAc (0-30%) over 15 min to afford the product, tert-butyl (5-(2-(1,3-dimethyl-1H-pyrazol-4-yl)pyrazolo[5,1-b]thiazole-7-carboxamido)-6-methylpyridin-3-yl)carbamate, as a tan solid (301.0 mg). LCMS (ESI): mass calcd. for C₂₂H₂₅N₇O₃S, 467.2; m/z found, 468.1 [M+H]⁺; ¹H NMR (400 MHz, DMSO-d6) δ ppm 1.49 (s, 9H), 2.32 (s, 3H), 2.39 (s, 3H), 3.80 (s, 3H), 7.98 (br s, 1H), 8.08 (s, 1H), 8.36 (d, J=1.00 Hz, 1H), 8.39 (s, 1H), 8.51 (s, 1H), 9.56 (br s, 1H), 9.85 (s, 1H).

### Step b: N-(5-amino-2-methylpyridin-3-yl)-2-(1,3-dimethyl-1H-pyrazol-4-yl)pyrazolo[5,1-b]thiazole-7-carboxamide

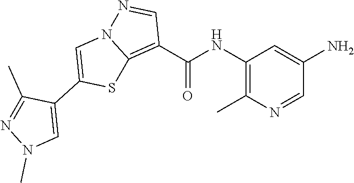 [SMILES: Cc1ncc(N)cc1NC(=O)c1cnn2cc(-c3cn(C)nc3C)sc12]

To a suspension of tert-butyl (5-(2-(1,3-dimethyl-1H-pyrazol-4-yl)pyrazolo[5,1-b]thiazole-7-carboxamido)-6-methylpyridin-3-yl)carbamate (300 mg, 0.64 mmol) in DCM (10 mL) was added HCl (4M in dioxane) (4 mL, 4 M, 16 mmol). The reaction mixture was stirred at 20° C. for 6 h. All solvents were removed in vacuo. The residue was dried under high vacuum to afford the HCl salt of the product, N-(5-amino-2-methylpyridin-3-yl)-2-(1,3-dimethyl-1H-pyrazol-4-yl)pyrazolo[5,1-b]thiazole-7-carboxamide, as a tan solid (quantitative yield). The product was used without further purification. LCMS (ESI): mass calcd. for C₁₇H₁₇N₇OS, 367.1; m/z found, 368.1 [M+H]⁺; ¹H NMR (400 MHz, DMSO-d6) δ ppm 1.59 (s, 3H), 2.34 (s, 3H), 2.53 (s, 3H), 6.14 (br s, 3H), 7.75 (d, J=1.00 Hz, 1H), 7.84 (d, J=1.00 Hz, 1H), 8.09 (s, 1H), 8.42 (s, 1H), 8.68 (s, 1H), 10.38 (s, 1H).

### Step c: 2-(1,3-dimethyl-1H-pyrazol-4-yl)-N-(5-(1-isopropylazetidine-3-carboxamido)-2-methylpyridin-3-yl)pyrazolo[5,1-b]thiazole-7-carboxamide

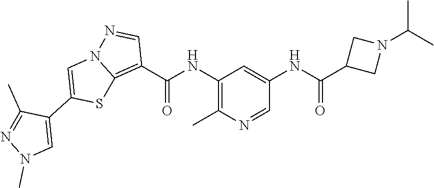 [SMILES: Cc1ncc(NC(=O)C2CN(C(C)C)C2)cc1NC(=O)c1cnn2cc(-c3cn(C)nc3C)sc12]

To a mixture of N-(5-amino-2-methylpyridin-3-yl)-2-(1,3-dimethyl-1H-pyrazol-4-yl)pyrazolo[5,1-b]thiazole-7-carboxamide hydrochloride (60 mg, 0.15 mmol) and 1-isopropylazetidine-3-carboxylic acid (31.91 mg, 0.22 mmol) in pyridine (1.5 mL) was added EDCI (42.72 mg, 0.22 mmol). The reaction was stirred at 25° C. for 21 h. LCMS indicated the starting amino pyridine had been consumed. All solvents were removed in vacuo. The residue was taken up in DMSO (2 mL) and submitted to the purification group for isolation to afford the product, 2-(1,3-dimethyl-1H-pyrazol-4-yl)-N-(5-(1-isopropylazetidine-3-carboxamido)-2-methylpyridin-3-yl)pyrazolo[5,1-b]thiazole-7-carboxamide, as a tan solid (33.6 mg). LCMS (ESI): mass calcd. for C₂₄H₂₈N₈O₂S, 492.2; m/z found, 493.1 [M+H]⁺; ¹H NMR (400 MHz, DMSO-d6) δ ppm 0.81 (d, J=1.00 Hz, 6H), 2.24 (quin, J=1.00 Hz, 1H), 2.34 (s, 3H), 2.41 (s, 3H), 3.11 (t, J=1.00 Hz, 2H), 3.26 (quin, J=1.00 Hz, 1H), 3.38 (t, J=1.00 Hz, 2H), 3.81 (s, 3H), 8.08 (s, 1H), 8.16 (d, J=1.00 Hz, 1H), 8.37 (s, 1H), 8.46-8.58 (m, 2H), 9.83 (s, 1H), 10.09 (s, 1H).

### Example 355: 2-(1,3-dimethyl-1H-pyrazol-4-yl)-N-(5-(1-isopropylpyrrolidine-3-carboxamido)-2-methylpyridin-3-yl)pyrazolo[5,1-b]thiazole-7-carboxamide

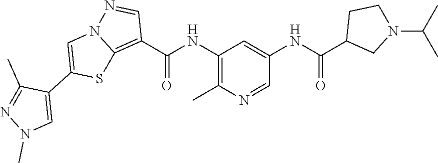 [SMILES: Cc1ncc(NC(=O)C2CCN(C(C)C)C2)cc1NC(=O)c1cnn2cc(-c3cn(C)nc3C)sc12]

To a mixture of N-(5-amino-2-methylpyridin-3-yl)-2-(1-(2-methoxyethyl)-1H-pyrazol-4-yl)pyrazolo[5,1-b]thiazole-7-carboxamide hydrochloride (60 mg, 0.14 mmol) and 1-(propan-2-yl)pyrrolidine-3-carboxylic acid (35 mg, 0.22 mmol) in pyridine (1.5 mL) was added EDCI (42.72 mg, 0.22 mmol). The reaction was stirred at 25° C. for 18 h. All solvents were removed in vacuo. The residue was dissolved in DMSO (1.5 mL) and was purified by HPLC (H₂O/ACN, 0.16% NH₄OH, XBridge 19×100) to afford the product, 2-(1,3-dimethyl-1H-pyrazol-4-yl)-N-(5-(1-isopropylpyrrolidine-3-carboxamido)-2-methylpyridin-3-yl)pyrazolo[5,1-b]thiazole-7-carboxamide, as a tan solid (40.0 mg). LCMS (ESI): mass calcd. for C₂₅H₃₀N₈O₂S, 506.2; m/z found, 507.2 [M+H]⁺; ¹H NMR (400 MHz, DMSO-d6) δ ppm 1.02 (d, J=1.00 Hz, 3H) 1.04 (d, J=1.00 Hz, 3H) 1.90-2.02 (m, 2H) 2.35 (s, 3H) 2.36-2.39 (m, 1H) 2.40 (s, 3H) 2.42-2.48 (m, 1H) 2.52-2.57 (m, 1H) 2.69 (q, J=1.00 Hz, 1H) 2.94 (t, J=1.00 Hz, 1H) 3.03 (quin, J=1.00 Hz, 1H) 3.79 (s, 3H) 8.07 (s, 1H) 8.15 (d, J=1.00 Hz, 1H) 8.39 (s, 1H) 8.52 (s, 2H) 9.83 (s, 1H) 10.08 (s, 1H).

### Example 356: 2-(1,5-dimethyl-1H-pyrazol-4-yl)-N-(5-(1-isopropylpyrrolidine-3-carboxamido)-2-methylpyridin-3-yl)pyrazolo[5,1-b]thiazole-7-carboxamide

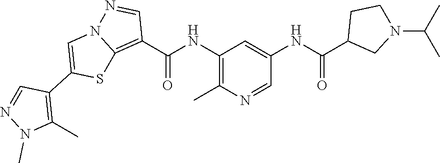 [SMILES: Cc1ncc(NC(=O)C2CCN(C(C)C)C2)cc1NC(=O)c1cnn2cc(-c3cnn(C)c3C)sc12]

### Step a: tert-butyl (5-(2-(1,5-dimethyl-1H-pyrazol-4-yl)pyrazolo[5,1-b]thiazole-7-carboxamido)-6-methylpyridin-3-yl)carbamate

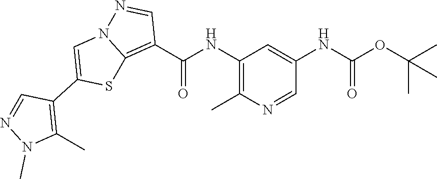 [SMILES: Cc1ncc(NC(=O)OC(C)(C)C)cc1NC(=O)c1cnn2cc(-c3cnn(C)c3C)sc12]

To a mixture of tert-butyl (5-(6-bromo-[1,2,3]triazolo[1,5-a]pyridine-3-carboxamido)-6-methylpyridin-3-yl)carbamate (850 mg, 1.88 mmol) and 1,5-dimethyl-1H-pyrazole-4-boronic acid, pinacol ester (500.82 mg, 2.26 mmol) in 1,4-dioxane (10 mL) was added a solution of K₂CO₃ (779 mg, 5.64 mmol) in water (2 mL) followed by the addition of 1,1′-bis(diphenylphosphino)ferrocene-palladium(II)dichloride dichlormethane complex (306.92 mg, 0.38 mmol). The reaction was thoroughly flushed with argon before being capped and heated at 100° C. for 23.5 h. The reaction was diluted with MeOH (25 mL) and silica gel (10 g) was added. All solvents were removed in vacuo. The silica gel mesh was loaded on a Redi Sep Rf silica gel cartridge (40 g) eluting with CH₂Cl₂ over 5 min then MeOH/CH₂Cl₂ (0-30%) over 15 min to afford a brown solid. The solid was dissolved in 20% MeOH/CH₂Cl₂ (25 mL) and silica gel (5 g) was added. All solvents were removed in vacuo. The silica gel mesh was loaded on a Redi Sep Rf silica gel cartridge (40 g) eluting with EtOAc over 5 min then MeOH/EtOAc (0-30%) over 15 min to afford the product, tert-butyl (5-(2-(1,5-dimethyl-1H-pyrazol-4-yl)pyrazolo[5,1-b]thiazole-7-carboxamido)-6-methylpyridin-3-yl)carbamate, as a tan solid (437.4 mg). LCMS (ESI): mass calcd. for C₂₂H₂₅N₇O₃S, 467.2; m/z found, 468.1 [M+H]⁺; ¹H NMR (400 MHz, DMSO-d6) δ ppm 1.49 (s, 9H), 2.36 (s, 3H), 2.44 (s, 3H), 3.80 (s, 3H), 7.72 (s, 1H), 7.98 (br s, 1H), 8.36 (d, J=1.00 Hz, 1H), 8.44 (s, 1H), 8.52 (s, 1H), 9.56 (br s, 1H), 9.85 (s, 1H).

### Step b: N-(5-amino-2-methylpyridin-3-yl)-2-(1,5-dimethyl-1H-pyrazol-4-yl)pyrazolo[5,1-b]thiazole-7-carboxamide

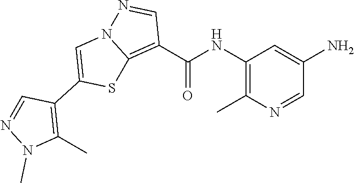 [SMILES: Cc1ncc(N)cc1NC(=O)c1cnn2cc(-c3cnn(C)c3C)sc12]

To a suspension of tert-butyl (5-(2-(1,5-dimethyl-1H-pyrazol-4-yl)pyrazolo[5,1-b]thiazole-7-carboxamido)-6-methylpyridin-3-yl)carbamate (437 mg, 0.94 mmol) in DCM (10 mL) was added HCl (4M in dioxane) (5.84 mL, 4 M, 23.37 mmol). The reaction was stirred at 25° C. for 21.5 h. All solvents were revised in vacuo. The residue was dried under high vacuum to afford the HCl salt of the product, N-(5-amino-2-methylpyridin-3-yl)-2-(1,5-dimethyl-1H-pyrazol-4-yl)pyrazolo[5,1-b]thiazole-7-carboxamide, as a tan solid (quantitative yield). The product was used without further purification. LCMS (ESI): mass calcd. for C₁₇H₁₇N₇OS, 367.1; m/z found, 368.1 [M+H]⁺; ¹H NMR (400 MHz, DMSO-d6) δ ppm 2.44 (s, 3H), 2.53 (s, 3H), 3.81 (s, 3H), 6.25 (br s, 3H), 7.70 (s, 1H), 7.78 (d, J=1.00 Hz, 1H), 7.85 (d, J=1.00 Hz, 1H), 8.48 (s, 1H), 8.70 (s, 1H), 10.38 (s, 1H).

### Step c: 2-(1,5-dimethyl-1H-pyrazol-4-yl)-N-(5-(1-isopropylpyrrolidine-3-carboxamido)-2-methylpyridin-3-yl)pyrazolo[5,1-b]thiazole-7-carboxamide

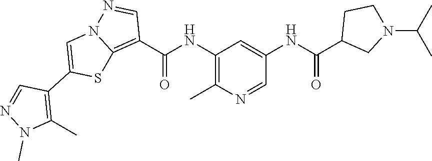 [SMILES: Cc1ncc(NC(=O)C2CCN(C(C)C)C2)cc1NC(=O)c1cnn2cc(-c3cnn(C)c3C)sc12]

To a mixture of N-(5-amino-2-methylpyridin-3-yl)-2-(1-(2-methoxyethyl)-1H-pyrazol-4-yl)pyrazolo[5,1-b]thiazole-7-carboxamide hydrochloride (60 mg, 0.14 mmol) and 1-(propan-2-yl)pyrrolidine-3-carboxylic acid (35 mg, 0.22 mmol) in pyridine (1.5 mL) was added EDCI (42.72 mg, 0.22 mmol)). The reaction was stirred at 25° C. for 18 h. All solvents were removed in vacuo. The residue was dissolved in DMSO (1.5 mL) and was purified by HPLC (H₂O/ACN, 0.16% NH₄OH, XBridge 19×100) to afford the product, 2-(1,5-dimethyl-1H-pyrazol-4-yl)-N-(5-(1-isopropylpyrrolidine-3-carboxamido)-2-methylpyridin-3-yl)pyrazolo[5,1-b]thiazole-7-carboxamide, as a tan solid (39.8 mg). LCMS (ESI): mass calcd. for C₂₅H₃₀N₈O₂S, 506.2; m/z found, 507.2 [M+H]⁺; ¹H NMR (400 MHz, DMSO-d6) δ ppm 1.01 (d, J=1.00 Hz, 3H), 1.04 (d, J=1.00 Hz, 3H), 1.87-2.03 (m, 2H), 2.35 (quin, J=1.00 Hz, 1H), 2.40 (s, 3H), 2.44 (s, 3H), 2.45-2.49 (m, 1H), 2.52-2.57 (m, 1H), 2.69 (q, J=1.00 Hz, 1H), 2.94 (t, J=1.00 Hz, 1H), 3.03 (quin, J=1.00 Hz, 1H), 3.80 (s, 3H), 7.71 (s, 1H), 8.15 (d, J=1.00 Hz, 1H), 8.45 (s, 1H), 8.51 (d, J=1.00 Hz, 1H), 8.53 (s, 1H), 9.84 (s, 1H), 10.09 (s, 1H).

### Example 357: 2-(1,5-dimethyl-1H-pyrazol-4-yl)-N-(5-(1-isopropylazetidine-3-carboxamido)-2-methylpyridin-3-yl)pyrazolo[5,1-b]thiazole-7-carboxamide

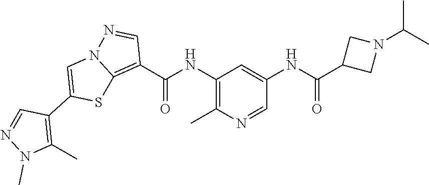 [SMILES: Cc1ncc(NC(=O)C2CN(C(C)C)C2)cc1NC(=O)c1cnn2cc(-c3cnn(C)c3C)sc12]

To a mixture of N-(5-amino-2-methylpyridin-3-yl)-2-(1-(2-methoxyethyl)-1H-pyrazol-4-yl)pyrazolo[5,1-b]thiazole-7-carboxamide hydrochloride (60 mg, 0.14 mmol) and 1-isopropylazetidine-3-carboxylic acid (31.9 mg, 0.22 mmol) in pyridine (1.5 mL) was added EDCI (42.72 mg, 0.22 mmol). The reaction was stirred at 25° C. for 18 h. LCMS indicated the starting amino pyridine had been consumed. All solvents were removed in vacuo. The residue was dissolved in DMSO (1.5 mL) and submitted to the purification group for isolation to afford the product, 2-(1,5-dimethyl-1H-pyrazol-4-yl)-N-(5-(1-isopropylazetidine-3-carboxamido)-2-methylpyridin-3-yl)pyrazolo[5,1-b]thiazole-7-carboxamide, as a tan solid (36.7 mg). LCMS (ESI): mass calcd. for C₂₄H₂₈N₈O₂S, 492.2; m/z found, 493.2 [M+H]⁺; ¹H NMR (400 MHz, DMSO-d6) δ ppm 0.83 (d, J=1.00 Hz, 6H), 2.25 (quin, J=1.00 Hz, 1H), 2.40 (s, 3H), 2.44 (s, 3H), 3.10 (t, J=1.00 Hz, 2H), 3.26 (quin, J=1.00 Hz, 1H), 3.38 (t, J=1.00 Hz, 2H), 3.80 (s, 3H), 7.71 (s, 1H), 8.16 (d, J=1.00 Hz, 1H), 8.44 (s, 1H), 8.50-8.56 (m, 2H), 9.84 (s, 1H), 10.08 (s, 1H).

### Example 358. N-(5-((2-(2-azabicyclo[2.2.2]octan-2-yl)ethyl)sulfonamido)-2-methylpyridin-3-yl)-2-(1-methyl-1H-pyrazol-4-yl)pyrazolo[5,1-b]thiazole-7-carboxamide

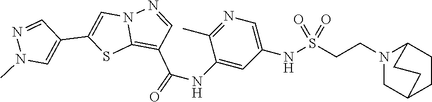 [SMILES: Cc1ncc(NS(=O)(=O)CCN2CC3CCC2CC3)cc1NC(=O)c1cnn2cc(-c3cnn(C)c3)sc12]

A suspension of N-(5-amino-2-methylpyridin-3-yl)-2-(1-methyl-1H-pyrazol-4-yl)pyrazolo[5,1-b]thiazole-7-carboxamide (50 mg, 0.1 mmol) in DCM under nitrogen was added N-methyl morpholine (44.5 μL, 0.4 mmol). The mixture was chilled on an ice bath and 2-chloroethanesulfonyl chloride (68 μL, 0.11 mmol) was added. The reaction was allowed to warm slowly to rt in the ice bath overnight. An additional 1.5 mL DCM was added and the reaction was stirred overnight. An additional 2 equivalents of N-methyl morpholine and 1 equivalent of 2-chloroethanesulfonyl chloride were added. After 3 h 4 equivalents of N-methyl morpholine and 2 equivalents of 2-chloroethanesulfonyl chloride were added. After 2 h water was added and the mixture was stirred vigorously, then let stand until the layers cleared. The layers were separated. The aqueous was frozen and lyophilized. To the crude product (a mixture of the desired chloride and salts) was added a solution of 2-azabicyclo[2.2.2]octane (56 mg, 0.51 mmol) in MeOH (3 mL) and the mixture was stirred at rt and TEA (150 uL) was added. The reaction was filtered and purified by prep-HPLC, 5%-25% MeCN/water/10 mM NH₄OH to yield N-(5-((2-(2-azabicyclo[2.2.2]octan-2-yl)ethyl)sulfonamido)-2-methylpyridin-3-yl)-2-(1-methyl-1H-pyrazol-4-yl)pyrazolo[5,1-b]thiazole-7-carboxamide (6 mg, 10%). LCMS (ESI): mass calcd. for C₂₅H₃₀N₈O₃S₂, 554.7; m/z found, 555.3 [M+H]⁺. ¹H NMR (DMSO-d6) δ: 9.84 (s, 1H), 8.57 (s, 1H), 8.49 (s, 1H), 8.19 (s, 1H), 8.14 (d, J=2.0 Hz, 1H), 7.87 (s, 1H), 7.66 (d, J=1.5 Hz, 1H), 3.88 (s, 3H), 3.18 (br s, 2H), 2.77-2.87 (m, 2H), 2.54 (d, J=2.4 Hz, 2H), 2.46-2.49 (m, 1H), 2.39 (s, 3H), 1.73-1.85 (m, 2H), 1.31-1.67 (m, 8H).

### Example 359. N-(5-(N-(2-(2,2-dimethylpyrrolidin-1-yl)ethyl)sulfamoyl)-2-methylpyridin-3-yl)-2-(1-methyl-1H-pyrazol-4-yl)pyrazolo[5,1-b]thiazole-7-carboxamide

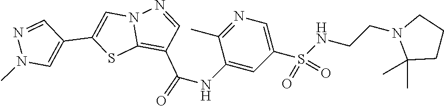 [SMILES: Cc1ncc(S(=O)(=O)NCCN2CCCC2(C)C)cc1NC(=O)c1cnn2cc(-c3cnn(C)c3)sc12]

### Step a: 5-(benzylthio)-2-methylpyridin-3-amine

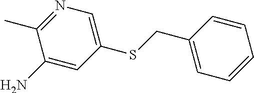 [SMILES: Cc1ncc(SCc2ccccc2)cc1N]

A mixture of 5-bromo-2-methylpyridin-3-amine (250 mg, 1.34 mmol), benzyl mercaptan (0.19 mL, 1.06 g/mL, 1.6 mmol), and DIPEA (0.46 mL, 0.75 g/mL, 2.67 mmol) in toluene (5 mL) in a microwave vial was sparged with nitrogen for 10 min, and tris(dibenzylideneacetone)dipalladium(0) (37 mg, 0.04 mmol) and 4,5-bis(diphenylphosphino)-9,9-dimethylxanthene (46 mg, 0.08 mmol) was added, the vial was capped and heated to 120° C. in the microwave for 15 h. The reaction was filtered and concentrated to a bright orange oil, which was purified by flash column, 0-5% MeOH/DCM to yield 5-(benzylthio)-2-methylpyridin-3-amine (299 mg, 97%) as a pale yellow solid. LCMS (ESI): mass calcd. for C₁₃H₁₄N2S, 230.3; m/z found, 231.1 [M+H]⁺.

### Step b: tert-butyl (5-(benzylthio)-2-methylpyridin-3-yl)carbamate

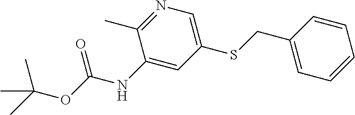 [SMILES: Cc1ncc(SCc2ccccc2)cc1NC(=O)OC(C)(C)C]

To a solution of 5-(benzylthio)-2-methylpyridin-3-amine (299 mg, 1.3 mmol) in THE (3 mL) was added sodium bis(trimethylsilyl)amide (2.6 mL, 1 M, 2.6 mmol) slowly dropwise and the resulting deep purple solution stirred at rt for 30 min. Di-tert-butyl pyrocarbonate (283 mg, 1.3 mmol) in 2 mL THE was added dropwise and the reaction was stirred at rt. After 3.5 h the reaction was concentrated and partitioned between EtOAc and water. The layers were separated and the aqueous extracted with EtOAc. The combined organics were washed with brine, filtered through cotton, concentrated, and purified by flash column, 0-50% EtOAc/heptane to yield tert-butyl (5-(benzylthio)-2-methylpyridin-3-yl)carbamate (123 mg, 29%). LCMS (ESI): mass calcd. for C₁₈H₂₂N₂O₂S, 330.5; m/z found, 331.1 [M+H]⁺.

### Step c: tert-butyl (5-(N-(2-(2,2-dimethylpyrrolidin-1-yl)ethyl)sulfamoyl)-2-methylpyridin-3-yl)carbamate

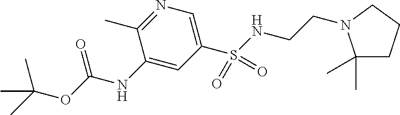 [SMILES: Cc1ncc(S(=O)(=O)NCCN2CCCC2(C)C)cc1NC(=O)OC(C)(C)C]

To an ice-bath chilled solution of tert-butyl (5-(benzylthio)-2-methylpyridin-3-yl)carbamate (123 mg, 0.37 mmol) in MeCN (1.2 mL), water (0.093 mL), and AcOH (0.056 mL, 0.98 mmol) was added 1,3-dichloro-5,5-dimethylhydantoin (147 mg, 0.74 mmol) portion-wise. The mixture was stirred cold for 15 min, then an ice bath chilled solution of 2-(2,2-dimethylpyrrolidin-1-yl)ethan-1-amine (159 mg, 1.12 mmol) in MeCN (1 mL) and TEA (0.2 mL, 1.49 mmol) was added. The reaction was stirred cold for 5 min, the ice bath was removed, and the reaction was stirred at rt for 1.5 h. An additional 0.1 mL TEA was added and the reaction was stirred for 1 h. The reaction was filtered and purified by prep-HPLC, 37%-57% MeCN/water/10 mM NH₄OH to yield tert-butyl (5-(N-(2-(2,2-dimethylpyrrolidin-1-yl)ethyl)sulfamoyl)-2-methylpyridin-3-yl)carbamate (78 mg, 51%) as a pale yellow solid. LCMS (ESI): mass calcd. for C₁₉H₃₂N₄O₄S, 412.6; m/z found, 413.2 [M+H]⁺.

### Step d: 5-amino-N-(2-(2,2-dimethylpyrrolidin-1-yl)ethyl)-6-methylpyridine-3-sulfonamide

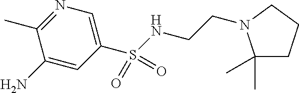 [SMILES: Cc1ncc(S(=O)(=O)NCCN2CCCC2(C)C)cc1N]

To a solution of tert-butyl (5-(N-(2-(2,2-dimethylpyrrolidin-1-yl)ethyl)sulfamoyl)-2-methylpyridin-3-yl)carbamate (78 mg, 0.19 mmol) in DCM (2 mL) was added HCl (4M in dioxane, 1 mL, 4 mmol) and the reaction was stirred at rt for 1.5 h. The reaction was diluted with ether and filtered to yield a tacky yellow solid which was dissolved in MeOH, concentrated, and dried under high vacuum overnight to yield the crude 5-amino-N-(2-(2,2-dimethylpyrrolidin-1-yl)ethyl)-6-methylpyridine-3-sulfonamide (68 mg, 103%) which was used without purification in the next step. LCMS (ESI): mass calcd. for C₁₄H₂₄N₄O₂S, 312.4; m/z found, 313.1 [M+H]⁺.

### Step e: ethyl 2-(1-methyl-1H-pyrazol-4-yl)pyrazolo[5,1-b]thiazole-7-carboxylate

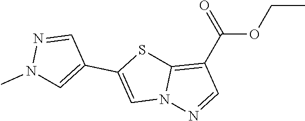 [SMILES: CCOC(=O)c1cnn2cc(-c3cnn(C)c3)sc12]

To a mixture of ethyl 2-bromopyrazolo[5,1-b]thiazole-7-carboxylate (500 mg, 1.82 mmol), 1-methyl-4-(4,4,5,5-tetramethyl-1,3,2-dioxaborolan-2-yl)-1H-pyrazole (454 mg, 2.18 mmol), and Cs₂CO₃ (888 mg, 2.73 mmol) under nitrogen in a 25 mL microwave vial was added 1,4-dioxane (10 mL) and water (2 mL). The mixture was sparged with nitrogen for 10 minutes, PdCl₂(dppf) (130 mg, 0.18 mmol) was added, the vial was capped and heated at 130° C. in the microwave for 1 h. The reaction was diluted with water and EtOAc, the mixture was filtered through Celite, the layers were separated, and the aqueous was extracted 2×EtOAc. The combined organics were washed 2× with brine, filtered through cotton, and concentrated to a brown oil, which was purified by flash column, 0-100% EtOAc/heptane to yield ethyl 2-(1-methyl-1H-pyrazol-4-yl)pyrazolo[5,1-b]thiazole-7-carboxylate (170 mg, 33%) as a reddish-tan solid. LCMS (ESI): mass calcd. for C₁₂H₁₂N₄O₂S, 276.3; m/z found, 277.1 [M+H]⁺.

### Step f: 2-(1-methyl-1H-pyrazol-4-yl)pyrazolo[5,1-b]thiazole-7-carboxylic acid

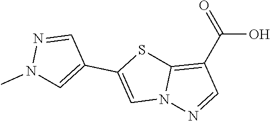 [SMILES: Cn1cc(-c2cn3ncc(C(=O)O)c3s2)cn1]

To a solution of ethyl 2-(1-methyl-1H-pyrazol-4-yl)pyrazolo[5,1-b]thiazole-7-carboxylate (170 mg, 0.62 mmol) in 1,4-dioxane (10 mL) was added a solution of LiOH (44 mg, 1.85 mmol) in water (2 mL) and the reaction was heated to 60° C. for 3 h. The reaction was cooled, acidified with 1N HCl and concentrated to a slurry which was filtered, washed with water, and dried under vacuum in the filter funnel to yield 2-(1-methyl-1H-pyrazol-4-yl)pyrazolo[5,1-b]thiazole-7-carboxylic acid (143 mg, 94%) as a tan powder. LCMS (ESI): mass calcd. for C₁₀H₈N₄O₂S, 248.3; m/z found, 249.1 [M+H]⁺.

### Step g: 2-(1-methyl-1H-pyrazol-4-yl)pyrazolo[5,1-b]thiazole-7-carbonyl chloride

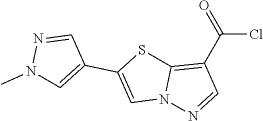 [SMILES: Cn1cc(-c2cn3ncc(C(=O)Cl)c3s2)cn1]

A suspension of 2-(1-methyl-1H-pyrazol-4-yl)pyrazolo[5,1-b]thiazole-7-carboxylic acid (108 mg, 0.44 mmol) in thionyl chloride (2 mL, 27.57 mmol) was heated to 70° C. After 2 h the reaction was concentrated to yield 2-(1-methyl-1H-pyrazol-4-yl)pyrazolo[5,1-b]thiazole-7-carbonyl chloride (140 mg, 81%) as a tan solid which was used without further purification in the next step.

### Step h: N-(5-(N-(2-(2,2-dimethylpyrrolidin-1-yl)ethyl)sulfamoyl)-2-methylpyridin-3-yl)-2-(1-methyl-1H-pyrazol-4-yl)pyrazolo[5,1-b]thiazole-7-carboxamide

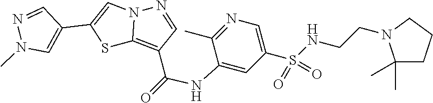 [SMILES: Cc1ncc(S(=O)(=O)NCCN2CCCC2(C)C)cc1NC(=O)c1cnn2cc(-c3cnn(C)c3)sc12]

To a suspension of 5-amino-N-(2-(2,2-dimethylpyrrolidin-1-yl)ethyl)-6-methylpyridine-3-sulfonamide (32 mg, 0.092 mmol) and 2-(1-methyl-1H-pyrazol-4-yl)pyrazolo[5,1-b]thiazole-7-carbonyl chloride (40 mg, 0.1 mmol) in THE (2 mL) was added TEA (51 μL, 0.37 mmol) to yield a milky mixture which was stirred at rt for 10 min then at 65° C. for 5 h. The reaction was cooled to rt, concentrated, and purified by prep-HPLC, 25%-45% MeCN/water/10 mM NH₄OH to yield N-(5-(N-(2-(2,2-dimethylpyrrolidin-1-yl)ethyl)sulfamoyl)-2-methylpyridin-3-yl)-2-(1-methyl-1H-pyrazol-4-yl)pyrazolo[5,1-b]thiazole-7-carboxamide (23 mg, 46%). LCMS (ESI): mass calcd. for C₂₄H₃₀N₈O₃S₂, 542.7; m/z found, 543.3 [M+H]⁺. ¹H NMR (DMSO-d6) δ: 10.01 (s, 1H), 8.68 (d, J=2.4 Hz, 1H), 8.60 (s, 1H), 8.54 (s, 1H), 8.25-8.30 (m, 1H), 8.20 (s, 1H), 7.88 (s, 1H), 7.67-7.78 (m, 1H), 3.88 (s, 3H), 2.87 (br s, 2H), 2.53-2.61 (m, 5H), 2.37 (br t, J=6.8 Hz, 2H), 1.55-1.65 (m, 2H), 1.43-1.51 (m, 2H), 0.85 (s, 6H).

### Example 360. N-(5-((2-(2,2-dimethylpyrrolidin-1-yl)ethyl)carbamoyl)-2-methylpyridin-3-yl)-2-(3-methoxypropyl)pyrazolo[5,1-b]thiazole-7-carboxamide and Example 361: (E)-N-(5-((2-(2,2-dimethylpyrrolidin-1-yl)ethyl)carbamoyl)-2-methylpyridin-3-yl)-2-(3-methoxyprop-1-en-1-yl)pyrazolo[5,1-b]thiazole-7-carboxamide

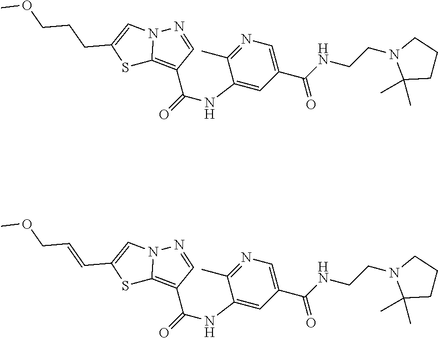 [SMILES: COCCCc1cn2ncc(C(=O)Nc3cc(C(=O)NCCN4CCCC4(C)C)cnc3C)c2s1 ; COC/C=C/c1cn2ncc(C(=O)Nc3cc(C(=O)NCCN4CCCC4(C)C)cnc3C)c2s1]

### Step a: ethyl (E)-2-(3-methoxyprop-1-en-1-yl)pyrazolo[5,1-b]thiazole-7-carboxylate

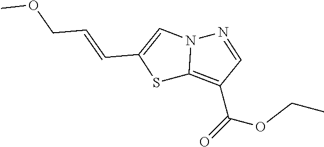 [SMILES: CCOC(=O)c1cnn2cc(/C=C/COC)sc12]

A mixture of ethyl 2-bromopyrazolo[5,1-b]thiazole-7-carboxylate (216 mg, 0.79 mmol), (E)-2-(3-methoxy-1-propen-1-yl)-4,4,5,5-tetramethyl-1,3,2-dioxaborolane [165059-42-7](194 mg, 0.98 mmol) and Cs₂CO₃ (435 mg, 1.33 mmol) in 1,4-dioxane (5 mL) and water (1 mL) under nitrogen was sparged with nitrogen for 10 minutes. PdCl₂(dppf)·CH₂Cl₂ (64 mg, 0.079 mmol) was added and the reaction was heated to 100° C. for 1.5 h. The reaction was cooled to rt and poured into sat'd NH₄C₁/brine and extracted 3×EtOAc. The combined organics were washed with brine, filtered through cotton, concentrated and purified by flash column, 0-50% EtOAc/heptane to yield ethyl (E)-2-(3-methoxyprop-1-en-1-yl)pyrazolo[5,1-b]thiazole-7-carboxylate (169 mg, 81%). LCMS (ESI): mass calcd. for C₁₂H₁₄N₂O₃S, 266.3; m/z found, 267.1 [M+H]⁺.

### Step b: ethyl 2-(3-methoxypropyl)pyrazolo[5,1-b]thiazole-7-carboxylate and ethyl (E)-2-(3-methoxyprop-1-en-1-yl)pyrazolo[5,1-b]thiazole-7-carboxylate

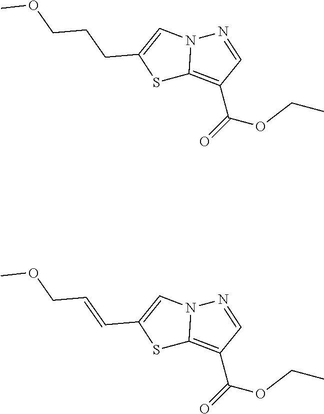

A mixture of ethyl (E)-2-(3-methoxyprop-1-en-1-yl)pyrazolo[5,1-b]thiazole-7-carboxylate (169 mg, 0.63 mmol) and Pd/C (10%, 68 mg, 0.063 mmol) in EtOH (5 mL) under vacuum in a round bottom flask equipped with a septum was placed under a hydrogen atmosphere via balloon, and the reaction was stirred at rt overnight. The flask was evacuated and back-filled with nitrogen. The reaction was filtered concentrated to a crude mixture of ethyl 2-(3-methoxypropyl)pyrazolo[5,1-b]thiazole-7-carboxylate and ethyl (E)-2-(3-methoxyprop-1-en-1-yl)pyrazolo[5,1-b]thiazole-7-carboxylate (170 mg, 100%) which was used in next step without purification. LCMS (ESI): mass calcd. for C₁₂H₁₆N₂O₃S, 268.1; m/z found, 269.0 [M+H]⁺. LCMS (ESI): mass calcd. for C₁₂H₁₄N₂O₃S, 266.1; m/z found, 267.0 [M+H]⁺.

### Step c: 2-(3-methoxypropyl)pyrazolo[5,1-b]thiazole-7-carboxylic acid and (E)-2-(3-methoxyprop-1-en-1-yl)pyrazolo[5,1-b]thiazole-7-carboxylic acid

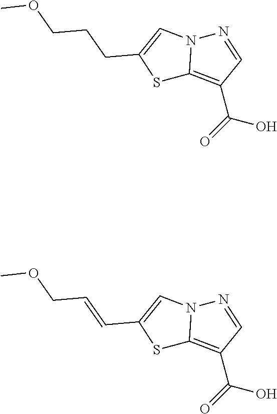 [SMILES: COCCCc1cn2ncc(C(=O)O)c2s1 ; COC/C=C/c1cn2ncc(C(=O)O)c2s1]

To a solution of the crude product from the previous reaction (170 mg, 0.63 mmol) in 1,4-dioxane (10 mL) was added a solution of LiOH (46 mg, 1.9 mmol) in water (2 mL) and the reaction was heated to 60° C. overnight. The reaction was cooled to rt, acidified with 1N HCl (2 mL), concentrated to remove dioxane, and extracted 3×EtOAc. The combined organics were washed with brine, filtered through cotton, concentrated to a waxy orange solid, and dried under high vacuum to yield a crude mixture of 2-(3-methoxypropyl)pyrazolo[5,1-b]thiazole-7-carboxylic acid and (E)-2-(3-methoxyprop-1-en-1-yl)pyrazolo[5,1-b]thiazole-7-carboxylic acid (110 mg, 72%) which was used in next step without purification. LCMS (ESI): mass calcd. for C₁₀H₁₀N₂O₃S, 238.3; m/z found, 239.0 [M+H]⁺. LCMS (ESI): mass calcd. for C₁₀H₁₂N₂O₃S, 240.3; m/z found, 241.0 [M+H]⁺.

### Step d: 2-(3-methoxypropyl)pyrazolo[5,1-b]thiazole-7-carbonyl chloride and (E)-2-(3-methoxyprop-1-en-1-yl)pyrazolo[5,1-b]thiazole-7-carbonyl chloride

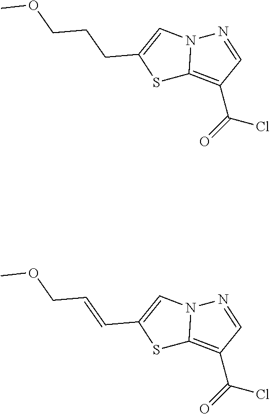 [SMILES: COCCCc1cn2ncc(C(=O)Cl)c2s1 ; COC/C=C/c1cn2ncc(C(=O)Cl)c2s1]

A suspension of the crude mixture from the previous reaction (110 mg, 0.46 mmol) in DCM (5 mL) was treated with SOCl₂ (0.33 mL) and the reaction was heated to 40° C. for 30 min. The reaction was cooled and concentrated to an orange oil. Heptane was added and stripped down three times to give a solid, which was dried under high vacuum to yield 2-(3-methoxypropyl)pyrazolo[5,1-b]thiazole-7-carbonyl chloride and (E)-2-(3-methoxyprop-1-en-1-yl)pyrazolo[5,1-b]thiazole-7-carbonyl chloride (100 mg, 84%) as a gummy solid which was used in next step without purification.

### Step e: N-(5-((2-(2,2-dimethylpyrrolidin-1-yl)ethyl)carbamoyl)-2-methylpyridin-3-yl)-2-(3-methoxypropyl)pyrazolo[5,1-b]thiazole-7-carboxamide and (E)-N-(5-((2-(2,2-dimethylpyrrolidin-1-yl)ethyl)carbamoyl)-2-methylpyridin-3-yl)-2-(3-methoxyprop-1-en-1-yl)pyrazolo[5,1-b]thiazole-7-carboxamide

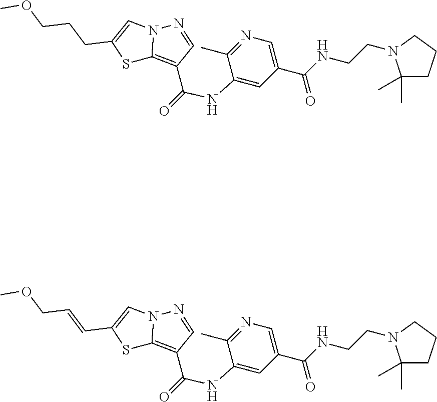 [SMILES: COCCCc1cn2ncc(C(=O)Nc3cc(C(=O)NCCN4CCCC4(C)C)cnc3C)c2s1 ; COC/C=C/c1cn2ncc(C(=O)Nc3cc(C(=O)NCCN4CCCC4(C)C)cnc3C)c2s1]

To a clear solution of 5-amino-N-(2-(2,2-dimethylpyrrolidin-1-yl)ethyl)-6-methylnicotinamide (30 mg, 0.11 mmol) and a mixture of 2-(3-methoxypropyl)pyrazolo[5,1-b]thiazole-7-carbonyl chloride and (E)-2-(3-methoxyprop-1-en-1-yl)pyrazolo[5,1-b]thiazole-7-carbonyl chloride (56.17 mg, 0.22 mmol) in THF (5 mL) was added TEA (60 μL, 0.43 mmol) and the reaction was stirred at rt overnight. Added 55 mg more of the mixture of mixture of 2-(3-methoxypropyl)pyrazolo[5,1-b]thiazole-7-carbonyl chloride and (E)-2-(3-methoxyprop-1-en-1-yl)pyrazolo[5,1-b]thiazole-7-carbonyl chloride, and 65 uL more TEA. The reaction was stirred at rt for 4 h then heated at 60° C. for 1 h. Then reaction was cooled to rt, concentrated, taken up in DMF/drops water, filtered, and purified by prep-HPLC, 22%-42% MeCN/water/10 mM NH₄OH to yield N-(5-((2-(2,2-dimethylpyrrolidin-1-yl)ethyl)carbamoyl)-2-methylpyridin-3-yl)-2-(3-methoxypropyl)pyrazolo[5,1-b]thiazole-7-carboxamide (3 mg, 5%); LCMS (ESI): mass calcd. for C₂₅H₃₄N₆O₃S, 498.7; m/z found, 499.3 [M+H]⁺; ¹H NMR (METHANOL-d4) δ: 8.77 (d, J=2.0 Hz, 1H), 8.37 (s, 1H), 8.30 (d, J=2.0 Hz, 1H), 7.89 (s, 1H), 3.56 (br t, J=6.8 Hz, 2H), 3.48 (t, J=6.1 Hz, 2H), 3.35 (s, 3H), 2.97-3.11 (m, 2H), 2.94 (t, J=7.6 Hz, 2H), 2.73-2.85 (m, 2H), 2.59 (s, 3H), 1.93-2.02 (m, 2H), 1.88 (br d, J=7.3 Hz, 2H), 1.72-1.80 (m, 2H), 1.11 (s, 6H) and (E)-N-(5-((2-(2,2-dimethylpyrrolidin-1-yl)ethyl)carbamoyl)-2-methylpyridin-3-yl)-2-(3-methoxyprop-1-en-1-yl)pyrazolo[5,1-b]thiazole-7-carboxamide (3 mg, 5%); LCMS (ESI): mass calcd. for C₂₅H₃₂N₆O₃S, 496.6; m/z found, 497.3 [M+H]⁺; ¹H NMR (METHANOL-d4) δ: 8.77 (d, J=2.0 Hz, 1H), 8.40 (s, 1H), 8.31 (d, J=2.0 Hz, 1H), 8.08 (s, 1H), 6.85 (d, J=15.7 Hz, 1H), 6.20 (dt, J=15.7, 5.4 Hz, 1H), 4.10 (dd, J=5.4, 1.5 Hz, 2H), 3.56 (br t, J=6.6 Hz, 2H), 3.39 (s, 3H), 2.97-3.11 (m, 2H), 2.71-2.84 (m, 2H), 2.60 (s, 3H), 1.88 (quin, J=7.3 Hz, 2H), 1.71-1.79 (m, 2H), 1.10 (s, 6H).

### Example 362. 2-(1-((3-(benzyloxy)isoxazol-5-yl)methyl)-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide

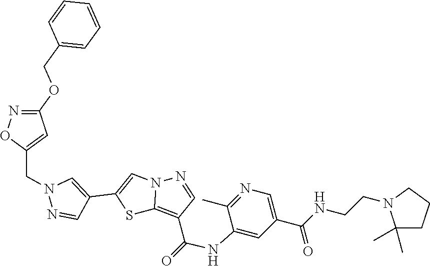 [SMILES: Cc1ncc(C(=O)NCCN2CCCC2(C)C)cc1NC(=O)c1cnn2cc(-c3cnn(Cc4cc(OCc5ccccc5)no4)c3)sc12]

### Step a: methyl 3-(benzyloxy)isoxazole-5-carboxylate

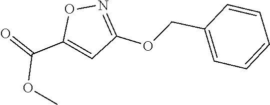 [SMILES: COC(=O)c1cc(OCc2ccccc2)no1]

To a solution of methyl 3-hydroxyisoxazole-5-carboxylate (250 mg, 1.75 mmol) in DMF (5 mL) under nitrogen at 0° C. was added Cs₂CO₃ (740 mg, 2.27 mmol). The mixture was stirred for 30 min, and benzyl bromide (0.31 mL, 2.62 mmol) was added drop-wise via syringe and the ice bath was removed. After 2 h the reaction was quenched with 1 N HCl (2.5 mL), poured into brine, and extracted 3×EtOAc. The combined extracts were washed 4×brine, filtered through cotton, concentrated and azeoptroped with heptane to yield the crude solid which was purified by flash column, 0-20% EtOAc/heptane to yield methyl 3-(benzyloxy)isoxazole-5-carboxylate (350 mg, 86%). LCMS (ESI): mass calcd. for C₁₂H₁₁NO₄, 233.2; m/z found, 234.1 [M+H]⁺.

### Step b: (3-(benzyloxy)isoxazol-5-yl)methanol

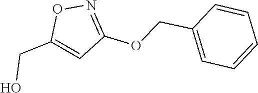 [SMILES: OCc1cc(OCc2ccccc2)no1]

To an ice-bath chilled solution of methyl 3-(benzyloxy)isoxazole-5-carboxylate (350 mg, 1.5 mmol) in MeOH (10 mL) under nitrogen was added NaBH₄ (68 mg, 1.8 mmol) portion-wise. The ice bath was removed and the reaction was stirred at rt. After 2.5 h, added another 65 mg NaBH₄ and stirred for 1 h. The reaction was quenched with 1N HCl (1 mL) and concentrated. To the residue was added sat'd NH₄Cl and the mixture was extracted 3×EtOAc. The combined organics were washed with brine, filtered through cotton, and concentrated to (3-(benzyloxy)isoxazol-5-yl)methanol (310 mg, 101%) as a clear oil. LCMS (ESI): mass calcd. for C₁₁H₁₁NO₃, 205.2; m/z found, 206.1 [M+H]⁺.

### Step c: (3-(benzyloxy)isoxazol-5-yl)methyl methanesulfonate

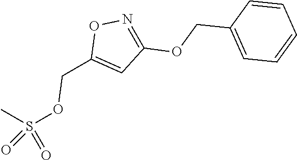 [SMILES: CS(=O)(=O)OCc1cc(OCc2ccccc2)no1]

To an ice-bath chilled solution of (3-(benzyloxy)isoxazol-5-yl)methanol (310 mg, 1.51 mmol) and TEA (0.31 mL, 2.27 mmol) in DCM (10 mL) under nitrogen was added methanesulfonyl chloride (0.13 mL, 1.66 mmol) slowly dropwise by syringe and the reaction was stirred cold for 1 h. The cold reaction solution was washed sequentially with ice-cold water, ice-cold 0.2 N HCl, and brine, filtered through cotton, and concentrated to (3-(benzyloxy)isoxazol-5-yl)methyl methanesulfonate (409 mg, 96%) as a hazy oil which crystallizes to a white solid. LCMS (ESI): mass calcd. for C₁₂H₁₃NO₅S, 283.3; m/z found, 284.0 [M+H]⁺.

### Step d: 3-(benzyloxy)-5-((4-bromo-1H-pyrazol-1-yl)methyl)isoxazole

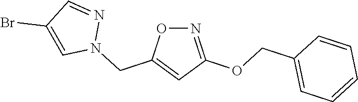 [SMILES: Brc1cnn(Cc2cc(OCc3ccccc3)no2)c1]

To a suspension of NaH (60% dispersion in mineral oil, 63 mg, 1.57 mmol) in 4 mL THF was added a solution of 4-bromo-1H-pyrazole (197 mg, 1.31 mmol) in 3 mL THE dropwise by syringe (gas evolution), and the reaction was stirred at rt for 1.5 h. A solution of (3-(benzyloxy)isoxazol-5-yl)methyl methanesulfonate (409 mg, 1.44 mmol) in 3 mL THE was added dropwise by syringe and the reaction was stirred at rt for 35 min. The reaction was quenched with sat'd NH₄Cl and concentrated and the mixture was extracted 3×EtOAc. The combined organics were washed with brine, filtered through cotton, and concentrated to 3-(benzyloxy)-5-((4-bromo-1H-pyrazol-1-yl)methyl)isoxazole (486 mg, 111%) as a white solid which was used without purification in the next step. LCMS (ESI): mass calcd. for C₁₄H₁₂BrN₃O₂, 334.2; m/z found, 334.1/336.1 [M+H]⁺.

### Step e: 3-(benzyloxy)-5-((4-(4,4,5,5-tetramethyl-1,3,2-dioxaborolan-2-yl)-1H-pyrazol-1-yl)methyl)isoxazole

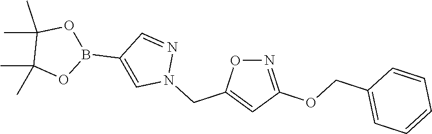 [SMILES: CC1(C)OB(c2cnn(Cc3cc(OCc4ccccc4)no3)c2)OC1(C)C]

KOAc (88 mg, 0.9 mmol) was heated at 100° C. under vacuum in a microwave vial for 3 h to ensure dehydration. The vial was cooled to rt and back-filled with nitrogen. 3-(Benzyloxy)-5-((4-bromo-1H-pyrazol-1-yl)methyl)isoxazole (150 mg, 0.45 mmol) and bis(pinacolato)diboron (125 mg, 0.49 mmol) were added, placed under vacuum, and back-filled with nitrogen. 1,4-dioxane (4 mL) was added and the mixture was sparged for 10 minutes with nitrogen. Chloro(2-dicyclohexylphosphino-2′,4′,6′-triisopropyl-1,1′-biphenyl)[2-(2′-amino-1,1′-biphenyl)]palladium(II) (18 mg, 0.022 mmol) was added and the reaction was heated to 100° C. in the microwave for 4 h. The reaction was filtered through a syringe filter with glass fiber pre-filter, rinsing with EtOAc. The resulting clear orange solution was concentrated and purified by flash column, 0-50% EtOAc/heptane to yield 3-(benzyloxy)-5-((4-(4,4,5,5-tetramethyl-1,3,2-dioxaborolan-2-yl)-1H-pyrazol-1-yl)methyl)isoxazole (94 mg, 48%). LCMS (ESI): mass calcd. for C₂₀H₂₄BN₃O₄, 381.2; m/z found, 382.1 [M+H]⁺.

### Step f: 2-(1-((3-(benzyloxy)isoxazol-5-yl)methyl)-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide

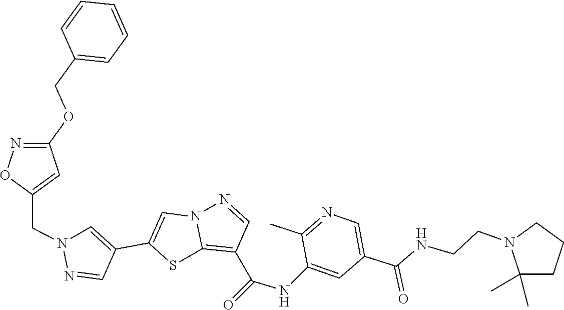 [SMILES: Cc1ncc(C(=O)NCCN2CCCC2(C)C)cc1NC(=O)c1cnn2cc(-c3cnn(Cc4cc(OCc5ccccc5)no4)c3)sc12]

A mixture of 2-bromo-N-(5-((2-(2,2-dimethylpyrrolidin-1-yl)ethyl)carbamoyl)-2-methylpyridin-3-yl)pyrazolo[5,1-b]thiazole-7-carboxamide (58 mg, 0.11 mmol), 3-(benzyloxy)-5-((4-(4,4,5,5-tetramethyl-1,3,2-dioxaborolan-2-yl)-1H-pyrazol-1-yl)methyl)isoxazole (55 mg, 0.13 mmol), Cs₂CO₃ (112 mg, 0.34 mmol), 1,4-dioxane (3.5 mL), and water (0.75 mL) was placed under vacuum and back-filled with nitrogen 2×, then the biphasic solution was sparged for 10 minutes with nitrogen. PdCl₂(dppf)·CH₂Cl₂ (9 mg, 0.011 mmol) was added and the reaction was heated at 130° C. in the microwave for 1 h. An additional 40 mg 3-(benzyloxy)-5-((4-(4,4,5,5-tetramethyl-1,3,2-dioxaborolan-2-yl)-1H-pyrazol-1-yl)methyl)isoxazole was dissolved in 0.75 mL dioxane and placed under vacuum and back-filled with nitrogen 4×before transferring to the microwave vial via syringe. The reaction was returned to 130° C. in the microwave for 1 h. The reaction was poured into sat'd NH₄Cl and extracted 3×EtOAc. The combined organics were washed with brine, filtered through cotton, and concentrated to a brown oil. The glassware was washed with MeOH and the brown solution (which has some product by HPLCMS) was concentrated. The crude product was purified by prep-HPLC, 40%-60% MeCN/water/10 mM NH₄OH to yield 2-(1-((3-(benzyloxy)isoxazol-5-yl)methyl)-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide (14 mg, 18%). LCMS (ESI): mass calcd. for C₃₅H₃₇N₉O₄S, 679.8; m/z found, 680.3 [M+H]⁺. ¹H NMR (DMSO-d6) δ: 9.98 (s, 1H), 8.77 (d, J=2.0 Hz, 1H), 8.65 (s, 1H), 8.57 (t, J=5.6 Hz, 1H), 8.53 (s, 1H), 8.36 (s, 1H), 8.20 (d, J=1.5 Hz, 1H), 7.98 (s, 1H), 7.34-7.48 (m, 5H), 6.32 (s, 1H), 5.52 (s, 2H), 5.23 (s, 2H), 2.76 (t, J=7.1 Hz, 2H), 1.63-1.74 (m, 2H), 1.51-1.58 (m, 2H), 0.92 (s, 6H).

### Example 363. N-(5-((2-(2,2-dimethylpyrrolidin-1-yl)ethyl)carbamoyl)-2-methylpyridin-3-yl)-2-(1-((3-hydroxyisoxazol-5-yl)methyl)-1H-pyrazol-4-yl)pyrazolo[5,1-b]thiazole-7-carboxamide

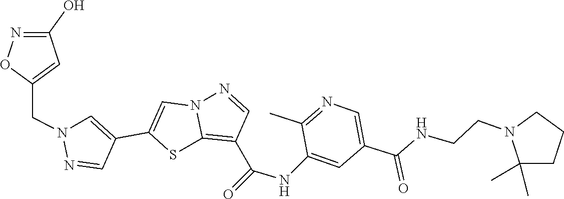 [SMILES: Cc1ncc(C(=O)NCCN2CCCC2(C)C)cc1NC(=O)c1cnn2cc(-c3cnn(Cc4cc(O)no4)c3)sc12]

To 2-(1-((3-(benzyloxy)isoxazol-5-yl)methyl)-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide (11 mg, 0.016 mmol) was added hydrobromic acid (33 wt % in HOAc, 1 mL, 5.51 mmol). The mixture was sonicated to produce an amber solution. After 30 min the reaction was stripped down on the rotovap, then chilled and basified with 10% NH₄OH. MeCN was added and stripped down to gummy solid which was purified by prep-HPLC, 6%-26% MeCN/water/10 mM NH₄OH to yield N-(5-((2-(2,2-dimethylpyrrolidin-1-yl)ethyl)carbamoyl)-2-methylpyridin-3-yl)-2-(1-((3-hydroxyisoxazol-5-yl)methyl)-1H-pyrazol-4-yl)pyrazolo[5,1-b]thiazole-7-carboxamide (7 mg, 73%). LCMS (ESI): mass calcd. for C₂₈H₃₁N₉O₄S, 589.7; m/z found, 590.3 [M+H]⁺. ¹H NMR (METHANOL-d4, 400 MHz) δ 8.78 (d, 1H, J=2.0 Hz), 8.41 (s, 1H), 8.35 (d, 1H, J=2.0 Hz), 8.28 (s, 1H), 8.15 (s, 1H), 7.87 (s, 1H), 5.76 (s, 1H), 5.35 (s, 2H), 3.66 (t, 2H, J=6.6 Hz), 3.04 (br t, 2H, J=6.6 Hz), 2.62 (s, 3H), 2.0-2.1 (m, 2H), 1.9-1.9 (m, 2H), 1.24 (s, 6H).

### Example 364. N-(5-(2-(3,3-dimethylazetidin-1-yl)acetamido)-2-methylpyridin-3-yl)-2-(3,5-dimethylisoxazol-4-yl)pyrazolo[5,1-b]thiazole-7-carboxamide

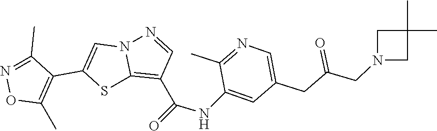 [SMILES: Cc1ncc(CC(=O)CN2CC(C)(C)C2)cc1NC(=O)c1cnn2cc(-c3c(C)noc3C)sc12]

### Step a: N-(5-amino-2-methylpyridin-3-yl)-2-bromopyrazolo[5,1-b]thiazole-7-carboxamide hydrochloride

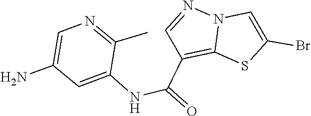 [SMILES: Cc1ncc(N)cc1NC(=O)c1cnn2cc(Br)sc12]

To a milky solution of tert-butyl (5-(2-bromopyrazolo[5,1-b]thiazole-7-carboxamido)-6-methylpyridin-3-yl)carbamate (2.02 g, 4.47 mmol) in DCM (15 mL) and MeOH (5 mL) was added HCl (4M in dioxane, 15 mL, 60 mmol), resulting in a thick precipitate and the reaction was stirred at rt overnight, concentrated, and dried under high vacuum to yield N-(5-amino-2-methylpyridin-3-yl)-2-bromopyrazolo[5,1-b]thiazole-7-carboxamide hydrochloride (1.9 g, 100%) which was used without purification in the next step. LCMS (ESI): mass calcd. for C₁₂H₁₀BrN₅OS, 352.2; m/z found, 352.0/354.0 [M+H]⁺.

### Step b: 2-bromo-N-(5-(2-chloroacetamido)-2-methylpyridin-3-yl)pyrazolo[5,1-b]thiazole-7-carboxamide

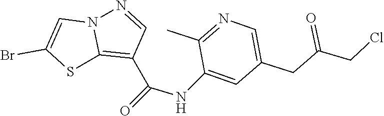 [SMILES: Cc1ncc(CC(=O)CCl)cc1NC(=O)c1cnn2cc(Br)sc12]

To a suspension of N-(5-amino-2-methylpyridin-3-yl)-2-bromopyrazolo[5,1-b]thiazole-7-carboxamide hydrochloride (1.9 g, 4.47 mmol) in DCM (35 mL) was added DIPEA (2.3 mL) to yield a hazy light amber mixture which was stirred for 10 minutes, then 2-chloroacetyl chloride (0.43 mL, 5.36 mmol) was added drop-wise to yield a thick mixture which was stirred at rt for 45 min. The reaction was poured into water with vigorous stirring and filtered, rinsing well with DCM and water and drying in the filter funnel to yield an off-white solid which was stripped down from MeCN to remove residual water and dried under high vac to yield 2-bromo-N-(5-(2-chloroacetamido)-2-methylpyridin-3-yl)pyrazolo[5,1-b]thiazole-7-carboxamide (1.97 g, 97%) which was used without further purification in the next step. LCMS (ESI): mass calcd. for C₁₄H₁₄BrClN₅O₂S, 428.7; m/z found, 427.9/429.9 [M+H]⁺.

### Step c: 2-bromo-N-(5-(2-(3,3-dimethylazetidin-1-yl)acetamido)-2-methylpyridin-3-yl)pyrazolo[5,1-b]thiazole-7-carboxamide

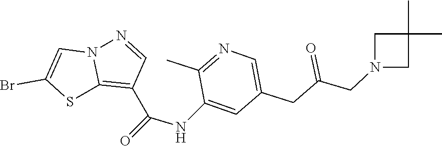 [SMILES: Cc1ncc(CC(=O)CN2CC(C)(C)C2)cc1NC(=O)c1cnn2cc(Br)sc12]

A mixture of 2-bromo-N-(5-(2-chloroacetamido)-2-methylpyridin-3-yl)pyrazolo[5,1-b]thiazole-7-carboxamide (100 mg, 0.22 mmol), 3,3-dimethylazetidine hydrochloride (30 mg, 0.24 mmol), and Cs₂CO₃ (159 mg, 0.49 mmol) in DMF (1 mL) under nitrogen in a 4 mL vial was heated to 50° C. overnight. The reaction was poured into ice water with vigorous stirring and a solid was collected, rinsed with ice water, dried under vacuum in the filter funnel, then stripped down with MeCN to remove residue water, and dried under high vacuum to yield 2-bromo-N-(5-(2-(3,3-dimethylazetidin-1-yl)acetamido)-2-methylpyridin-3-yl)pyrazolo[5,1-b]thiazole-7-carboxamide (90 mg, 64%) as a tan solid. LCMS (ESI): mass calcd. for C₁₉H₂₁BrN₆O₂S, 477.4; m/z found, 477.0/479.0 [M+H]⁺.

### Step d: N-(5-(2-(3,3-dimethylazetidin-1-yl)acetamido)-2-methylpyridin-3-yl)-2-(3,5-dimethylisoxazol-4-yl)pyrazolo[5,1-b]thiazole-7-carboxamide

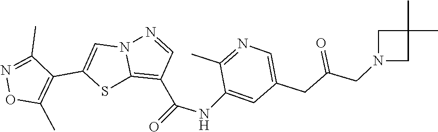 [SMILES: Cc1ncc(CC(=O)CN2CC(C)(C)C2)cc1NC(=O)c1cnn2cc(-c3c(C)noc3C)sc12]

A mixture of 2-bromo-N-(5-(2-(3,3-dimethylazetidin-1-yl)acetamido)-2-methylpyridin-3-yl)pyrazolo[5,1-b]thiazole-7-carboxamide (57 mg, 0.12 mmol), 3,5-dimethyl-4-(tetramethyl-1,3,2-dioxaborolan-2-yl)-1,2-oxazole (36 mg, 0.16 mmol), and Cs₂CO₃ (231 mg, 0.71 mmol) in 1,4-dioxane (4 mL) and water (1 mL) under nitrogen in a microwave vial was sparged with nitrogen for 10 minutes, and PdCl₂(dppf)·CH₂Cl₂ (10 mg, 0.012 mmol) was added, and the reaction was heated for 45 min in the microwave at 130° C. Added 20 mg more 3,5-dimethyl-4-(tetramethyl-1,3,2-dioxaborolan-2-yl)-1,2-oxazole and 10 mg more PdCl₂(dppf)·CH₂Cl₂, recapped vial, placed under vacuum and back-filled with nitrogen 3×, and returned to microwave at 130° C. for 1 h. The reaction mixture was stripped down with silica gel and dried under high vacuum overnight. The reaction was stirred with Si-trisamine for 30 min, filtered, and purified by prep-HPLC, 15%-35% MeCN/water/0.1% TFA to yield N-(5-(2-(3,3-dimethylazetidin-1-yl)acetamido)-2-methylpyridin-3-yl)-2-(3,5-dimethylisoxazol-4-yl)pyrazolo[5,1-b]thiazole-7-carboxamide (20 mg, 28%). LCMS (ESI): mass calcd. for C₂₄H₂₇N₇O₃S, 493.6; m/z found, 494.2 [M+H]⁺. ¹H NMR (METHANOL-d4) δ: 8.69 (d, J=2.4 Hz, 1H), 8.47 (s, 1H), 8.40 (d, J=2.4 Hz, 1H), 8.26 (s, 1H), 4.32 (s, 2H), 3.91-4.16 (m, 4H), 2.58 (s, 3H), 2.55 (s, 3H), 2.37 (s, 3H), 1.31-1.51 (m, 6H).

### Example 365. N-(5-(2-(2,6-trans-dimethylmorpholino)acetamido)-2-methylpyridin-3-yl)-2-(1H-pyrrol-3-yl)pyrazolo[5,1-b]thiazole-7-carboxamide

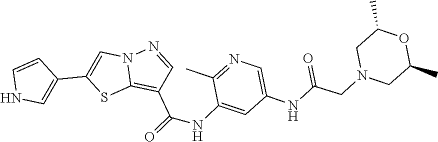 [SMILES: Cc1ncc(NC(=O)CN2C[C@H](C)O[C@@H](C)C2)cc1NC(=O)c1cnn2cc(-c3cc[nH]c3)sc12]

### Step a: tert-butyl (5-(2-bromopyrazolo[5,1-b]thiazole-7-carboxamido)-6-methylpyridin-3-yl)carbamate

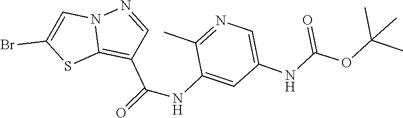 [SMILES: Cc1ncc(NC(=O)OC(C)(C)C)cc1NC(=O)c1cnn2cc(Br)sc12]

To a clear solution of tert-butyl (5-amino-6-methylpyridin-3-yl)carbamate (200 mg, 0.9 mmol) and 2-bromopyrazolo[5,1-b]thiazole-7-carbonyl chloride (262 mg, 0.99 mmol) in THE (6 mL) was added DIPEA (0.2 mL) and the reaction was stirred at rt for 1.5 h. The reaction was concentrated, taken up in DMF/drops water, filtered, and purified by prep-HPLC, 30%-50% MeCN/water/10 mM NH₄OH to yield tert-butyl (5-(2-bromopyrazolo[5,1-b]thiazole-7-carboxamido)-6-methylpyridin-3-yl)carbamate (348 mg, 86%). LCMS (ESI): mass calcd. for C₁₇H₁₈BrN₅O₃S, 452.3; m/z found, 452.0/454.0 [M+H]⁺.

### Step b: tert-butyl (5-(2-(1H-pyrrol-3-yl)pyrazolo[5,1-b]thiazole-7-carboxamido)-6-methylpyridin-3-yl)carbamate

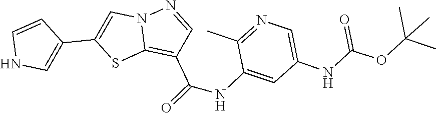 [SMILES: Cc1ncc(NC(=O)OC(C)(C)C)cc1NC(=O)c1cnn2cc(-c3cc[nH]c3)sc12]

A mixture of tert-butyl (5-(2-bromopyrazolo[5,1-b]thiazole-7-carboxamido)-6-methylpyridin-3-yl)carbamate (117 mg, 0.26 mmol), 3-(4,4,5,5-tetramethyl-1,3,2-dioxaborolan-2-yl)-1 h-pyrrole (68 mg, 0.35 mmol), and Cs₂CO₃ (267 mg, 0.82 mmol) in 1,4-dioxane (3.5 mL) and water (0.75 mL) under nitrogen was sparged with nitrogen for 15 minutes, PdCl₂(dppf) (21 mg, 0.026 mmol) was added, and the reaction was heated at 130° C. in the microwave for 40 min. An additional portion of 3-(4,4,5,5-tetramethyl-1,3,2-dioxaborolan-2-yl)-1 h-pyrrole and PdCl₂(dppf) was added, the vial was recapped, evacuated, and back-filled with nitrogen 3× and returned to the microwave at 130° C. for 1 h. The reaction mixture was stripped down with silica gel for a dry-load cartridge and purified by flash column, 0-10% 9:1 MeOH/NH₄OH in DCM to yield tert-butyl (5-(2-(1H-pyrrol-3-yl)pyrazolo[5,1-b]thiazole-7-carboxamido)-6-methylpyridin-3-yl)carbamate as a tan solid (58 mg, 51%). LCMS (ESI): mass calcd. for C₂₁H₂₂N₆O₃S, 438.5; m/z found, 439.1 [M+H]⁺.

### Step c: N-(5-amino-2-methylpyridin-3-yl)-2-(1H-pyrrol-3-yl)pyrazolo[5,1-b]thiazole-7-carboxamide hydrochloride

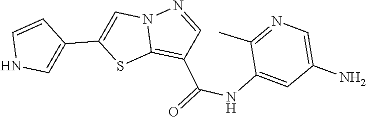 [SMILES: Cc1ncc(N)cc1NC(=O)c1cnn2cc(-c3cc[nH]c3)sc12]

A suspension of tert-butyl (5-(2-(1H-pyrrol-3-yl)pyrazolo[5,1-b]thiazole-7-carboxamido)-6-methylpyridin-3-yl)carbamate (73 mg, 0.17 mmol) in DCM (2 mL) was treated with HCl (4M in dioxane, 3 mL, 12 mmol). The mixture was sonicated and stirred at rt for 2 h then concentrated, stripped down from EtOAc to remove residual HCl, and dried under high vacuum overnight to yield N-(5-amino-2-methylpyridin-3-yl)-2-(1H-pyrrol-3-yl)pyrazolo[5,1-b]thiazole-7-carboxamide hydrochloride (71 mg, 114%) as a tan solid which was used without purification. LCMS (ESI): mass calcd. for C₁₆H₁₄N₆OS, 338.4; m/z found, 339.1 [M+H]⁺.

### Step d: N-(5-(2-chloroacetamido)-2-methylpyridin-3-yl)-2-(1H-pyrrol-3-yl)pyrazolo[5,1-b]thiazole-7-carboxamide

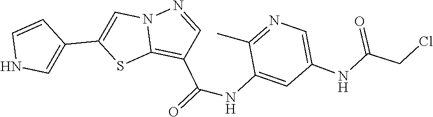 [SMILES: Cc1ncc(NC(=O)CCl)cc1NC(=O)c1cnn2cc(-c3cc[nH]c3)sc12]

To a suspension of N-(5-amino-2-methylpyridin-3-yl)-2-(1H-pyrrol-3-yl)pyrazolo[5,1-b]thiazole-7-carboxamide hydrochloride (71 mg, 0.19 mmol) in DCM (5 mL) and DIPEA (0.1 mL, 0.57 mmol) under nitrogen was added 2-chloroacetyl chloride (0.018 mL, 0.23 mmol) dropwise and the reaction was stirred at rt for 3 h. Another 30 uL DIPEA and 9 uL 2-chloroacetyl chloride was added and the reaction was stirred for 15 min. The reaction was concentrate, MeCN was added and reconcentrated 3× to remove residual 2-cloroacety chloride, then dried under high vacuum overnight to yield a viscous brown oil, which was dissolved in MeOH and stripped down with silica gel and purified by flash column, 0-10% MeOH/EtOAc to yield N-(5-(2-chloroacetamido)-2-methylpyridin-3-yl)-2-(1H-pyrrol-3-yl)pyrazolo[5,1-b]thiazole-7-carboxamide (41 mg, 52%) as an orange solid. LCMS (ESI): mass calcd. for C₁₈H₁₅ClN₆O₂S, 414.9; m/z found, 415.0/418.0 [M+H]⁺.

### Step e: N-(5-(2-(2,6-trans-dimethylmorpholino)acetamido)-2-methylpyridin-3-yl)-2-(1H-pyrrol-3-yl)pyrazolo[5,1-b]thiazole-7-carboxamide

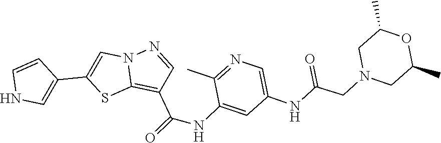 [SMILES: Cc1ncc(NC(=O)CN2C[C@H](C)O[C@@H](C)C2)cc1NC(=O)c1cnn2cc(-c3cc[nH]c3)sc12]

To a mixture of Cs₂CO₃ (49.5 mg, 0.15 mmol) and 2,6-trans-dimethylmorpholine (15.5 mg, 0.13 mmol) under nitrogen in a 20 mL vial was added a solution of N-(5-(2-chloroacetamido)-2-methylpyridin-3-yl)-2-(1H-pyrrol-3-yl)pyrazolo[5,1-b]thiazole-7-carboxamide (25 mg, 0.06 mmol) in DMF (2 mL) and the reaction was heated to 50° C. for 1 h. The reaction was filtered purified by prep-HPLC, 10%-45% MeCN/water/8% NH₄OH to yield N-(5-(2-(2,6-trans-dimethylmorpholino)acetamido)-2-methylpyridin-3-yl)-2-(1H-pyrrol-3-yl)pyrazolo[5,1-b]thiazole-7-carboxamide (7.2 mg, 25%). LCMS (ESI): mass calcd. for C₂₄H₂₇N₇O₃S, 493.6; m/z found, 494.1 [M+H]⁺. ¹H NMR (METHANOL-d4) δ: 8.60 (d, J=2.4 Hz, 1H), 8.34 (s, 1H), 8.23 (d, J=2.4 Hz, 1H), 8.02 (s, 1H), 7.14 (t, J=1.7 Hz, 1H), 6.80-6.85 (m, 1H), 6.42 (dd, J=2.7, 1.7 Hz, 1H), 4.10 (quind, J=6.3, 3.2 Hz, 2H), 3.18-3.25 (m, 1H), 3.04-3.11 (m, 1H), 2.62 (dd, J=11.0, 3.2 Hz, 2H), 2.50 (s, 3H), 2.32 (dd, J=10.8, 5.9 Hz, 2H), 1.29 (d, J=6.4 Hz, 6H).

### Example 366. N-(5-(2-(8-oxa-3-azabicyclo[3.2.1]octan-3-yl)acetamido)-2-methylpyridin-3-yl)-2-(1H-pyrrol-3-yl)pyrazolo[5,1-b]thiazole-7-carboxamide

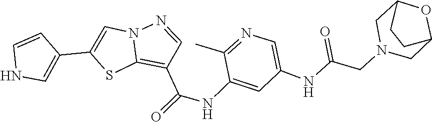 [SMILES: Cc1ncc(NC(=O)CN2CC3CCC(C2)O3)cc1NC(=O)c1cnn2cc(-c3cc[nH]c3)sc12]

To Cs₂CO₃ (47 mg, 0.14 mmol) and 8-oxa-3-azabicyclo[3.2.1]octane (9 mg, 0.08 mmol) under nitrogen in a 20 mL vial was added a solution of N-(5-(2-chloroacetamido)-2-methylpyridin-3-yl)-2-(1H-pyrrol-3-yl)pyrazolo[5,1-b]thiazole-7-carboxamide (17 mg, 0.041 mmol) in (2 mL) and the mixture was heated to 50° C. for 1 h. The reaction was filtered purified by prep-HPLC, 20%-50% MeCN/water/8% NH₄OH to yield N-(5-(2-(8-oxa-3-azabicyclo[3.2.1]octan-3-yl)acetamido)-2-methylpyridin-3-yl)-2-(1H-pyrrol-3-yl)pyrazolo[5,1-b]thiazole-7-carboxamide (7.4 mg, 37%). LCMS (ESI): mass calcd. for C₂₄H₂₅N₇O₃S, 491.6; m/z found, 490.2 [M−H]⁺. ¹H NMR (METHANOL-d4) δ: 8.59 (d, J=2.0 Hz, 1H), 8.34 (s, 1H), 8.22 (d, J=2.4 Hz, 1H), 8.03 (s, 1H), 7.14 (t, J=1.7 Hz, 1H), 6.83 (dd, J=2.9, 2.0 Hz, 1H), 6.42 (dd, J=2.9, 1.5 Hz, 1H), 4.31 (dd, J=4.4, 2.4 Hz, 2H), 3.15 (s, 2H), 2.71 (d, J=11.2 Hz, 2H), 2.47-2.54 (m, 5H), 2.10-2.18 (m, 2H), 1.88-1.97 (m, 2H).

### Example 367. N-(5-(2-(trans-2,6-dimethylmorpholino)acetamido)-2-methylpyridin-3-yl)-2-((E)-3-methoxyprop-1-en-1-yl)pyrazolo[5,1-b]thiazole-7-carboxamide

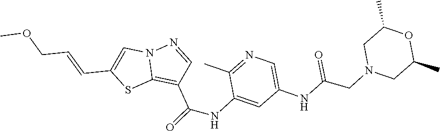

The title compound was prepared according to the procedure for Example 365, N-(5-(2-(2,6-trans-dimethylmorpholino)acetamido)-2-methylpyridin-3-yl)-2-(1H-pyrrol-3-yl)pyrazolo[5,1-b]thiazole-7-carboxamide. LCMS (ESI): mass calcd. for C₂₄H₃₀N₆O₄S, 498.6; m/z found, 499.1 [M+H]⁺. ¹H NMR (METHANOL-d4) δ: 8.59 (d, J=2.4 Hz, 1H), 8.38 (s, 1H), 8.23 (d, J=2.4 Hz, 1H), 8.07 (s, 1H), 6.85 (d, J=15.7 Hz, 1H), 6.20 (dt, J=16.0, 5.2 Hz, 1H), 4.05-4.16 (m, 4H), 3.39 (s, 3H), 3.18-3.24 (m, 1H), 3.04-3.10 (m, 1H), 2.62 (dd, J=11.0, 2.7 Hz, 2H), 2.49 (s, 3H), 2.31 (dd, J=11.0, 5.6 Hz, 2H), 1.29 (d, J=6.4 Hz, 6H).

### Example 368. N-(5-(2-(trans-2,6-dimethylmorpholino)acetamido)-2-methylpyridin-3-yl)-2-(2-methoxypyridin-3-yl)pyrazolo[5,1-b]thiazole-7-carboxamide

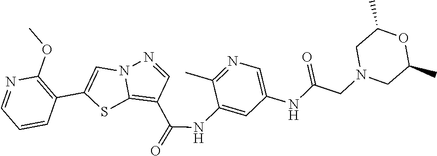 [SMILES: COc1ncccc1-c1cn2ncc(C(=O)Nc3cc(NC(=O)CN4C[C@H](C)O[C@@H](C)C4)cnc3C)c2s1]

### Step a: tert-butyl (5-(2-(2-methoxypyridin-3-yl)pyrazolo[5,1-b]thiazole-7-carboxamido)-6-methylpyridin-3-yl)carbamate

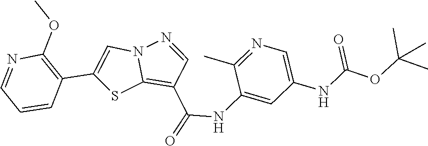 [SMILES: COc1ncccc1-c1cn2ncc(C(=O)Nc3cc(NC(=O)OC(C)(C)C)cnc3C)c2s1]

The title compound was prepared according to the procedure of Example 365, Step b, replacing 3-(4,4,5,5-tetramethyl-1,3,2-dioxaborolan-2-yl)-1H-pyrrole with 2-methoxypyridine-3-boronic acid. LCMS (ESI): mass calcd. for C₂₃H₂₄N₆O₄S, 480.5; m/z found, 481.1 [M+H]⁺.

### Steps c-e: N-(5-(2-(trans-2,6-dimethylmorpholino)acetamido)-2-methylpyridin-3-yl)-2-(2-methoxypyridin-3-yl)pyrazolo[5,1-b]thiazole-7-carboxamide

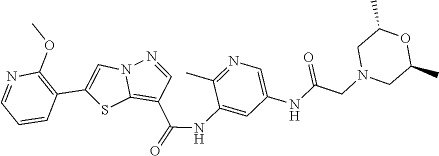 [SMILES: COc1ncccc1-c1cn2ncc(C(=O)Nc3cc(NC(=O)CN4C[C@H](C)O[C@@H](C)C4)cnc3C)c2s1]

The title compound was prepared from tert-butyl (5-(2-(2-methoxypyridin-3-yl)pyrazolo[5,1-b]thiazole-7-carboxamido)-6-methylpyridin-3-yl)carbamate according to Example 365, steps c-e. LCMS (ESI): mass calcd. for C₂₆H₂₉N₇O₄S, 535.6; m/z found, 536.2 [M+H]⁺. ¹H NMR (METHANOL-d4) δ: 8.64 (s, 1H), 8.59 (d, J=2.0 Hz, 1H), 8.43 (s, 1H), 8.26 (d, J=2.4 Hz, 1H), 8.19 (dd, J=5.1, 1.7 Hz, 1H), 8.07 (dd, J=7.3, 2.0 Hz, 1H), 7.10 (dd, J=7.3, 4.9 Hz, 1H), 4.06-4.15 (m, 5H), 3.18-3.25 (m, 1H), 3.04-3.11 (m, 1H), 2.63 (dd, J=11.0, 3.2 Hz, 2H), 2.51 (s, 3H), 2.32 (dd, J=11.0, 5.6 Hz, 2H), 1.29 (d, J=6.4 Hz, 6H).

### Example 369. N-(5-(2-(3,3-dimethylazetidin-1-yl)acetamido)-2-methylpyridin-3-yl)-2-(2-methoxypyridin-3-yl)pyrazolo[5,1-b]thiazole-7-carboxamide

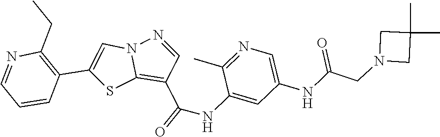 [SMILES: CCc1ncccc1-c1cn2ncc(C(=O)Nc3cc(NC(=O)CN4CC(C)(C)C4)cnc3C)c2s1]

To 2-methoxypyridine-3-boronic acid (17 mg, 0.11 mmol), Cs₂CO₃ (55 mg, 0.17 mmol), and PdCl₂(dppf)·CH₂Cl₂ (5 mg, 0.0057 mmol) under nitrogen in a capped microwave vial was added a solution of 2-bromo-N-(5-(2-(3,3-dimethylazetidin-1-yl)acetamido)-2-methylpyridin-3-yl)pyrazolo[5,1-b]thiazole-7-carboxamide (27 mg, 0.057 mmol) in 1,4-dioxane (2 mL) via syringe. Water (0.6 mL) was added, the mixture was sparged with nitrogen for 10 minutes, then heated to 130° C. in the microwave for 1 h. The reaction was filtered and purified by prep-HPLC, 30%-50% MeCN/water/10 mM NH₄OH to yield N-(5-(2-(3,3-dimethylazetidin-1-yl)acetamido)-2-methylpyridin-3-yl)-2-(2-methoxypyridin-3-yl)pyrazolo[5,1-b]thiazole-7-carboxamide (14 mg, 49%). LCMS (ESI): mass calcd. for C₂₅H₂₇N₇O₃S, 505.6; m/z found, 506.2 [M+H]⁺. ¹H NMR (METHANOL-d4) δ: 8.65 (s, 1H), 8.57 (d, J=2.4 Hz, 1H), 8.43 (s, 1H), 8.22 (d, J=2.4 Hz, 1H), 8.19 (dd, J=5.1, 1.7 Hz, 1H), 8.07 (dd, J=7.6, 1.7 Hz, 1H), 7.10 (dd, J=7.3, 4.9 Hz, 1H), 4.13 (s, 3H), 3.34 (s, 2H), 3.19 (s, 4H), 2.50 (s, 3H), 1.26 (s, 6H).

### Example 370. N-(5-(2-(trans-2,6-dimethylmorpholino)acetamido)-2-methylpyridin-3-yl)-2-(2-oxo-1,2-dihydropyridin-3-yl)pyrazolo[5,1-b]thiazole-7-carboxamide

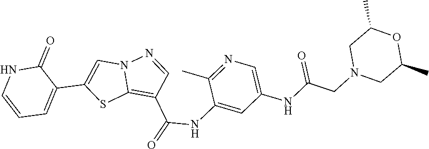 [SMILES: Cc1ncc(NC(=O)CN2C[C@H](C)O[C@@H](C)C2)cc1NC(=O)c1cnn2cc(-c3ccc[nH]c3=O)sc12]

### Step a: 2-bromo-N-(5-(2-(trans-2,6-dimethylmorpholino)acetamido)-2-methylpyridin-3-yl)pyrazolo[5,1-b]thiazole-7-carboxamide

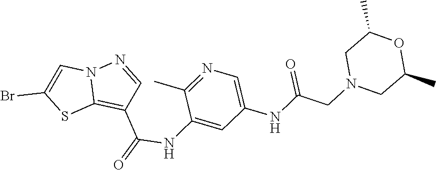 [SMILES: Cc1ncc(NC(=O)CN2C[C@H](C)O[C@@H](C)C2)cc1NC(=O)c1cnn2cc(Br)sc12]

To a mixture of 2-bromo-N-(5-(2-chloroacetamido)-2-methylpyridin-3-yl)pyrazolo[5,1-b]thiazole-7-carboxamide (508 mg, 1.18 mmol) and Cs₂CO₃ (700 mg, 2.15 mmol) under nitrogen was added DMF (7.5 mL) followed by 2,6-trans-dimethylmorpholine (200 mg, 1.74 mmol) and the reaction was heated to 50° C. for 45 min. The reaction was cooled to rt and pipetted into 30 mL of ice water with vigorous stirring. A tan solid was collected, rinsed with cold water, and dried under vacuum in the filter funnel. The crude product was recrystallized from MeCN to yield 2-bromo-N-(5-(2-(trans-2,6-dimethylmorpholino)acetamido)-2-methylpyridin-3-yl)pyrazolo[5,1-b]thiazole-7-carboxamide (362 mg, 56%). LCMS (ESI): mass calcd. for C₂₀H₂₃BrN₆O₃S, 507.4; m/z found, 507.1/509.1 [M+H]⁺.

### Step b: N-(5-(2-(trans-2,6-dimethylmorpholino)acetamido)-2-methylpyridin-3-yl)-2-(2-oxo-1,2-dihydropyridin-3-yl)pyrazolo[5,1-b]thiazole-7-carboxamide

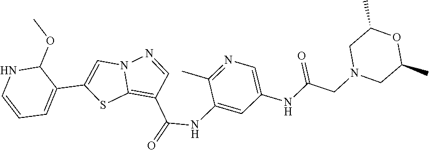 [SMILES: COC1NC=CC=C1c1cn2ncc(C(=O)Nc3cc(NC(=O)CN4C[C@H](C)O[C@@H](C)C4)cnc3C)c2s1]

A mixture of 2-bromo-N-(5-(2-(trans-2,6-dimethylmorpholino)acetamido)-2-methylpyridin-3-yl)pyrazolo[5,1-b]thiazole-7-carboxamide (50 mg, 0.092 mmol), (2-oxo-1,2-dihydropyridin-3-yl)boronic acid (34 mg, 0.24 mmol), Cs₂CO₃ (98 mg, 0.3 mmol), and PdCl₂(dppf)·CH₂Cl₂ (7.5 mg, 0.0092 mmol) in a capped 5 mL microwave vial was evacuated and back-filled with nitrogen. 1,4-dioxane (1.8 mL) and water (0.5 mL) were added (thick suspension) and the mixture was sparged with nitrogen for 10 minutes then heated in the microwave for 1 h at 130° C. Additional portions of (2-oxo-1,2-dihydropyridin-3-yl)boronic acid, PdCl₂(dppf)·CH₂Cl₂, and 0.5 mL DMF were added. The vial was capped, evacuated, back-filled with nitrogen, and sparged with nitrogen for 10 min, then returned to microwave at 130° C. for 1 h. The reaction was stirred with Si-trisamine for 25 min, filtered, and purified by prep-HPLC, 12%-40% MeCN/water/0.1% TFA to yield N-(5-(2-(trans-2,6-dimethylmorpholino)acetamido)-2-methylpyridin-3-yl)-2-(2-oxo-1,2-dihydropyridin-3-yl)pyrazolo[5,1-b]thiazole-7-carboxamide trifluoroacetate (11 mg, 19%). LCMS (ESI): mass calcd. for C₂₅H₂₇N₇O₄S, 521.6; m/z found, 522.3 [M+H]⁺. ¹H NMR (METHANOL-d4) δ: 8.81 (s, 1H), 8.71 (d, J=2.4 Hz, 1H), 8.44 (s, 1H), 8.42 (d, J=2.4 Hz, 1H), 8.15 (dd, J=7.3, 2.0 Hz, 1H), 7.52 (dd, J=6.4, 1.5 Hz, 1H), 6.55 (dd, J=7.3, 6.4 Hz, 1H), 4.23-4.32 (m, 2H), 4.00-4.18 (m, 2H), 3.15-3.25 (m, 2H), 2.59 (s, 3H), 1.36 (br d, J=6.4 Hz, 6H).

### Example 371. N-(5-(2-(trans-2,6-dimethylmorpholino)acetamido)-2-methylpyridin-3-yl)-2-(3-methyl-1H-pyrazol-4-yl)pyrazolo[5,1-b]thiazole-7-carboxamide

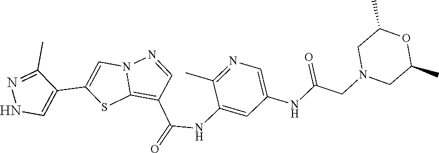 [SMILES: Cc1ncc(NC(=O)CN2C[C@H](C)O[C@@H](C)C2)cc1NC(=O)c1cnn2cc(-c3c[nH]nc3C)sc12]

The title compound was prepared from 2-bromo-N-(5-(2-(trans-2,6-dimethylmorpholino)acetamido)-2-methylpyridin-3-yl)pyrazolo[5,1-b]thiazole-7-carboxamide according to the procedure of Example 370 Step b, substituting 3-methyl-4-(4,4,5,5-tetramethyl-1,3,2-dioxaborolan-2-yl)-1H-pyrazole for (2-oxo-1,2-dihydropyridin-3-yl)boronic acid (10 mg, 21%). LCMS (ESI): mass calcd. for C₂₄H₂₈N₈O₃S, 508.6; m/z found, 509.1 [M+H]⁺. ¹H NMR (METHANOL-d4) δ: 8.59 (d, J=2.4 Hz, 1H), 8.40 (s, 1H), 8.25 (d, J=2.0 Hz, 1H), 8.13 (s, 1H), 7.71-8.00 (m, 1H), 4.05-4.15 (m, 2H), 3.18-3.25 (m, 1H), 3.04-3.11 (m, 1H), 2.62 (dd, J=11.0, 3.2 Hz, 2H), 2.46-2.52 (m, 1H), 2.45-2.54 (m, 5H), 2.32 (dd, J=11.0, 5.6 Hz, 2H), 1.29 (d, J=6.4 Hz, 6H).

### Example 372. 2-(6-amino-5-fluoropyridin-3-yl)-N-(5-(2-(trans-2,6-dimethylmorpholino)acetamido)-2-methylpyridin-3-yl)pyrazolo[5,1-b]thiazole-7-carboxamide

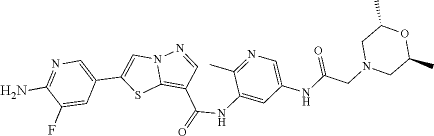 [SMILES: Cc1ncc(NC(=O)CN2C[C@H](C)O[C@@H](C)C2)cc1NC(=O)c1cnn2cc(-c3cnc(N)c(F)c3)sc12]

To a mixture of 2-bromo-N-(5-(2-(trans-2,6-dimethylmorpholino)acetamido)-2-methylpyridin-3-yl)pyrazolo[5,1-b]thiazole-7-carboxamide (52 mg, 0.095 mmol), 3-fluoro-5-(4,4,5,5-tetramethyl-1,3,2-dioxaborolan-2-yl)pyridin-2-amine (49 mg, 0.2 mmol), Cs₂CO₃ (99 mg, 0.31 mmol), and PdCl₂(dppf)·CH₂Cl₂ (27 mg, 0.033 mmol) under nitrogen in a capped 5 mL microwave vial was added 1,4-dioxane (1.5 mL), water (0.4 mL), and DMF (0.5 mL) and the mixture was sparged with nitrogen for 10 min then heated at 130° C. in the microwave for 1 h. The reaction was stirred with Si-trisamine, filtered, and purified by prep-HPLC, 14%-50% MeCN/water/10 mM NH₄OH to yield 2-(6-amino-5-fluoropyridin-3-yl)-N-(5-(2-(trans-2,6-dimethylmorpholino)acetamido)-2-methylpyridin-3-yl)pyrazolo[5,1-b]thiazole-7-carboxamide (26 mg, 51%). LCMS (ESI): mass calcd. for C₂₅H₂₇FN₈O₃S, 538.2; m/z found, 539.2 [M+H]⁺. ¹H NMR (METHANOL-d4) δ: 8.59 (d, J=2.0 Hz, 1H), 8.40 (s, 1H), 8.37 (s, 1H), 8.25 (d, J=2.0 Hz, 1H), 8.06 (d, J=1.5 Hz, 1H), 7.70 (dd, J=11.7, 2.0 Hz, 1H), 4.05-4.15 (m, 2H), 3.18-3.26 (m, 1H), 3.04-3.11 (m, 1H), 2.62 (dd, J=11.0, 3.2 Hz, 2H), 2.50 (s, 3H), 2.32 (dd, J=11.0, 5.6 Hz, 2H), 1.29 (d, J=6.4 Hz, 6H).

### Example 373. 2-(3,5-dimethyl-1H-pyrazol-4-yl)-N-(5-(2-(trans-2,6-dimethylmorpholino)acetamido)-2-methylpyridin-3-yl)pyrazolo[5,1-b]thiazole-7-carboxamide

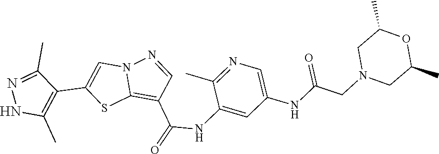 [SMILES: Cc1ncc(NC(=O)CN2C[C@H](C)O[C@@H](C)C2)cc1NC(=O)c1cnn2cc(-c3c(C)n[nH]c3C)sc12]

The title compound was prepared according to Example 370 Step b, substituting 3,5-dimethyl-4-(tetramethyl-1,3,2-dioxaborolan-2-yl)-1H-pyrazole for (2-oxo-1,2-dihydropyridin-3-yl)boronic acid (9 mg, 19%). LCMS (ESI): mass calcd. for C₂₅H₃₀N₈O₃S, 522.6; m/z found, 523.2 [M+H]⁺. ¹H NMR (METHANOL-d4) δ: 8.59 (d, J=2.4 Hz, 1H), 8.42 (s, 1H), 8.25 (d, J=2.4 Hz, 1H), 8.03 (s, 1H), 4.05-4.16 (m, 2H), 3.18-3.25 (m, 1H), 3.03-3.11 (m, 1H), 2.62 (dd, J=10.8, 2.9 Hz, 2H), 2.51 (s, 2H), 2.48-2.53 (m, 1H), 2.28-2.43 (m, 6H), 2.26-2.45 (m, 1H), 1.29 (d, J=6.8 Hz, 6H).

### Example 374. N-(5-(2-(trans-2,6-dimethylmorpholino)acetamido)-2-methylpyridin-3-yl)-2-(1H-pyrazol-3-yl)pyrazolo[5,1-b]thiazole-7-carboxamide

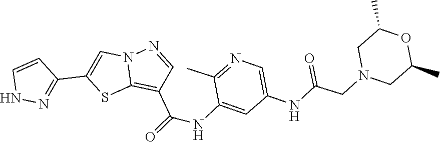 [SMILES: Cc1ncc(NC(=O)CN2C[C@H](C)O[C@@H](C)C2)cc1NC(=O)c1cnn2cc(-c3cc[nH]n3)sc12]

The title compound was prepared from 2-bromo-N-(5-(2-(trans-2,6-dimethylmorpholino)acetamido)-2-methylpyridin-3-yl)pyrazolo[5,1-b]thiazole-7-carboxamide according to the procedure of Example 370 Step b, substituting 3-(4,4,5,5-tetramethyl-1,3,2-dioxaborolan-2-yl)-1H-pyrazole for (2-oxo-1,2-dihydropyridin-3-yl)boronic acid (3 mg, 5%). LCMS (ESI): mass calcd. for C₂₃H₂₆N₈O₃S, 494.6; m/z found, 495.1 [M+H]⁺. ¹H NMR (METHANOL-d4) δ: 8.60 (d, J=2.4 Hz, 1H), 8.40-8.43 (m, 2H), 8.24 (d, J=2.4 Hz, 1H), 7.75 (d, J=2.0 Hz, 1H), 6.74 (d, J=2.4 Hz, 1H), 4.10 (quind, J=6.2, 2.9 Hz, 2H), 3.18-3.25 (m, 1H), 3.04-3.11 (m, 1H), 2.62 (dd, J=11.2, 2.9 Hz, 2H), 2.32 (dd, J=11.0, 5.6 Hz, 2H), 1.29 (d, J=6.8 Hz, 6H).

### Example 375. 2-(6,7-dihydro-5H-pyrazolo[5,1-b][1,3]oxazin-3-yl)-N-(5-(2-(trans-2,6-dimethylmorpholino)acetamido)-2-methylpyridin-3-yl)pyrazolo[5,1-b]thiazole-7-carboxamide

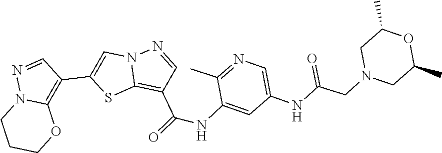 [SMILES: Cc1ncc(NC(=O)CN2C[C@H](C)O[C@@H](C)C2)cc1NC(=O)c1cnn2cc(-c3cnn4c3OCCC4)sc12]

The title compound was prepared from 2-bromo-N-(5-(2-(trans-2,6-dimethylmorpholino)acetamido)-2-methylpyridin-3-yl)pyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (18 mg, 33%). LCMS (ESI): mass calcd. for C₂₆H₃₀N₈O₄S, 550.6; m/z found, 551.2 [M+H]⁺. ¹H NMR (METHANOL-d4) δ: 8.60 (d, J=2.4 Hz, 1H), 8.35 (s, 1H), 8.23 (d, J=2.4 Hz, 1H), 8.06 (s, 1H), 7.68 (s, 1H), 4.49-4.54 (m, 2H), 4.19 (t, J=6.1 Hz, 2H), 4.10 (quind, J=6.2, 2.9 Hz, 2H), 3.18-3.25 (m, 1H), 3.04-3.11 (m, 1H), 2.62 (dd, J=11.0, 3.2 Hz, 2H), 2.50 (s, 3H), 2.27-2.40 (m, 4H), 1.29 (d, J=6.4 Hz, 6H).

### Example 376. (R)—N-(5-(2-(6-(hydroxymethyl)-2,2-dimethylmorpholino)acetamido)-2-methylpyridin-3-yl)-2-(1-methyl-1H-pyrazol-4-yl)pyrazolo[5,1-b]thiazole-7-carboxamide

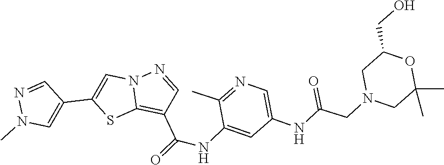

### Step a: (R)-2-bromo-N-(5-(2-(6-(hydroxymethyl)-2,2-dimethylmorpholino)acetamido)-2-methylpyridin-3-yl)pyrazolo[5,1-b]thiazole-7-carboxamide

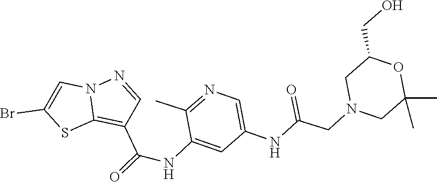 [SMILES: Cc1ncc(NC(=O)CN2C[C@H](CO)OC(C)(C)C2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with (R)-(6,6-dimethylmorpholin-2-yl)methanol hydrochloride (166 mg, 70%). LCMS (ESI): mass calcd. for C₂₁H₂₅BrN₆O₄S, 537.4; m/z found, 537.0/539.0 [M+H]⁺.

### Step b: (R)—N-(5-(2-(6-(hydroxymethyl)-2,2-dimethylmorpholino)acetamido)-2-methylpyridin-3-yl)-2-(1-methyl-1H-pyrazol-4-yl)pyrazolo[5,1-b]thiazole-7-carboxamide

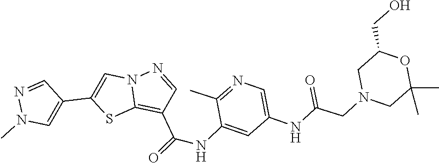

The title compound was prepared from (R)-2-bromo-N-(5-(2-(6-(hydroxymethyl)-2,2-dimethylmorpholino)acetamido)-2-methylpyridin-3-yl)pyrazolo[5,1-b]thiazole-7-carboxamide according to the procedure of Example 372, substituting 1-methylpyrazole-4-boronic acid, pinacol ester for 3-fluoro-5-(4,4,5,5-tetramethyl-1,3,2-dioxaborolan-2-yl)pyridin-2-amine (34 mg, 55%). LCMS (ESI): mass calcd. for C₂₅H₃₀N₈O₄S, 538.6; m/z found, 539.2 [M+H]⁺. ¹H NMR (METHANOL-d4, 400 MHz) δ: 8.74 (d, 1H, J=2.4 Hz), 8.46 (d, 1H, J=2.4 Hz), 8.41 (s, 1H), 8.25 (s, 1H), 8.03 (s, 1H), 7.82 (s, 1H), 4.1-4.2 (m, 1H), 4.0-4.1 (m, 2H), 3.94 (s, 3H), 3.6-3.7 (m, 2H), 3.4-3.6 (m, 2H), 2.99 (br t, 1H, J=11.5 Hz), 2.87 (br d, 1H, J=12.2 Hz), 2.60 (s, 3H), 1.50 (s, 3H), 1.31 (s, 3H).

### Example 377. (S)—N-(5-(2-(6-(hydroxymethyl)-2,2-dimethylmorpholino)acetamido)-2-methylpyridin-3-yl)-2-(1-methyl-1H-pyrazol-4-yl)pyrazolo[5,1-b]thiazole-7-carboxamide

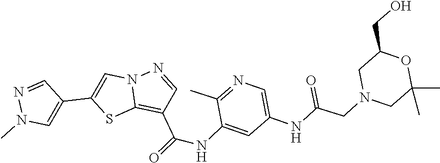

### Step a: (S)-2-bromo-N-(5-(2-(6-(hydroxymethyl)-2,2-dimethylmorpholino)acetamido)-2-methylpyridin-3-yl)pyrazolo[5,1-b]thiazole-7-carboxamide

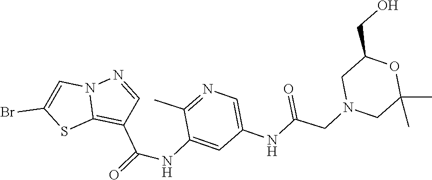 [SMILES: Cc1ncc(NC(=O)CN2C[C@@H](CO)OC(C)(C)C2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with (S)-(6,6-dimethylmorpholin-2-yl)methanol hydrochloride (181 mg, 76%). LCMS (ESI): mass calcd. for C₂₁H₂₅BrN₆O₄S, 537.4; m/z found, 537.1/539.1 [M+H]⁺.

### Step b: (S)—N-(5-(2-(6-(hydroxymethyl)-2,2-dimethylmorpholino)acetamido)-2-methylpyridin-3-yl)-2-(1-methyl-1H-pyrazol-4-yl)pyrazolo[5,1-b]thiazole-7-carboxamide

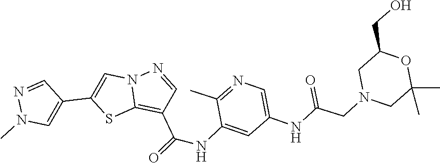

The title compound was prepared from (S)-2-bromo-N-(5-(2-(6-(hydroxymethyl)-2,2-dimethylmorpholino)acetamido)-2-methylpyridin-3-yl)pyrazolo[5,1-b]thiazole-7-carboxamide according to the procedure of Example 372, substituting 1-methylpyrazole-4-boronic acid, pinacol ester for 3-fluoro-5-(4,4,5,5-tetramethyl-1,3,2-dioxaborolan-2-yl)pyridin-2-amine (34 mg, 55%). LCMS (ESI): mass calcd. for C₂₅H₃₀N₈O₄S, 538.6; m/z found, 539.1 [M+H]⁺. ¹H NMR (METHANOL-d4) δ: 8.80 (br d, J=2.0 Hz, 1H), 8.53 (br d, J=2.0 Hz, 1H), 8.42 (s, 1H), 8.25 (s, 1H), 8.03 (s, 1H), 7.82 (s, 1H), 4.18-4.28 (m, 1H), 4.05-4.16 (m, 2H), 3.94 (s, 3H), 3.45-3.68 (m, 4H), 3.02 (br t, J=11.5 Hz, 1H), 2.84-2.94 (m, 1H), 2.59-2.67 (m, 3H), 1.50 (s, 3H), 1.32 (s, 3H).

### Example 378. N-(5-(2-(cis-2-(hydroxymethyl)-6-methylmorpholino)acetamido)-2-methylpyridin-3-yl)-2-(1-methyl-1H-pyrazol-4-yl)pyrazolo[5,1-b]thiazole-7-carboxamide

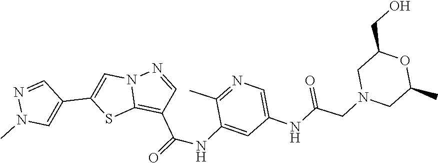

### Step a: 2-bromo-N-(5-(2-(cis-2-(hydroxymethyl)-6-methylmorpholino)acetamido)-2-methylpyridin-3-yl)pyrazolo[5,1-b]thiazole-7-carboxamide

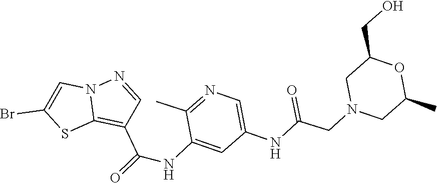 [SMILES: Cc1ncc(NC(=O)CN2C[C@@H](CO)O[C@@H](C)C2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with (cis-6-methylmorpholin-2-yl)methanol hydrochloride (185 mg, 76%). LCMS (ESI): mass calcd. for C₂₀H₂₀BrN₆O₄S, 523.4; m/z found, 523.1/525.1 [M+H]⁺.

### Step b: N-(5-(2-(cis-2-(hydroxymethyl)-6-methylmorpholino)acetamido)-2-methylpyridin-3-yl)-2-(1-methyl-1H-pyrazol-4-yl)pyrazolo[5,1-b]thiazole-7-carboxamide

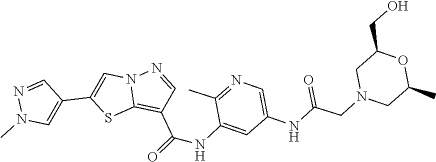

The title compound was prepared from (S)-2-bromo-N-(5-(2-(6-(hydroxymethyl)-2,2-dimethylmorpholino)acetamido)-2-methylpyridin-3-yl)pyrazolo[5,1-b]thiazole-7-carboxamide according to the procedure of Example 372, substituting 1-methylpyrazole-4-boronic acid, pinacol ester for 3-fluoro-5-(4,4,5,5-tetramethyl-1,3,2-dioxaborolan-2-yl)pyridin-2-amine (34 mg, 55%). LCMS (ESI): mass calcd. for C₂₄H₂₈N₈O₄S, 524.6; m/z found, 525.3 [M+H]⁺. ¹H NMR (METHANOL-d4) δ: 8.61 (d, J=2.4 Hz, 1H), 8.38 (s, 1H), 8.25 (d, J=2.0 Hz, 1H), 8.22 (s, 1H), 8.03 (s, 1H), 7.82 (s, 1H), 3.94 (s, 3H), 3.72-3.87 (m, 2H), 3.53 (qd, J=11.7, 5.4 Hz, 2H), 3.22 (d, J=1.0 Hz, 2H), 2.80-2.94 (m, 2H), 2.50 (s, 3H), 1.92-2.07 (m, 2H), 1.16 (d, J=6.4 Hz, 3H).

### Example 379. (R)—N-(5-(2-(6-(hydroxymethyl)-2,2-dimethylmorpholino)acetamido)-2-methylpyridin-3-yl)-2-(2-methoxypyridin-3-yl)pyrazolo[5,1-b]thiazole-7-carboxamide

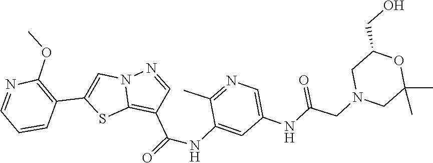 [SMILES: COc1ncccc1-c1cn2ncc(C(=O)Nc3cc(NC(=O)CN4C[C@H](CO)OC(C)(C)C4)cnc3C)c2s1]

The title compound was prepared from (R)-2-bromo-N-(5-(2-(6-(hydroxymethyl)-2,2-dimethylmorpholino)acetamido)-2-methylpyridin-3-yl)pyrazolo[5,1-b]thiazole-7-carboxamide

according to the procedure of Example 372, substituting 2-methoxypyridine-3-boronic acid for 3-fluoro-5-(4,4,5,5-tetramethyl-1,3,2-dioxaborolan-2-yl)pyridin-2-amine (29 mg, 56%). LCMS (ESI): mass calcd. for C₂₇H₃₁N₇O₅S, 565.7; m/z found, 566.2 [M+H]⁺. 1H NMR (METHANOL-d4) δ: 8.65 (s, 1H), 8.60 (s, 1H), 8.43 (s, 1H), 8.25 (s, 1H), 8.19 (br d, J=4.9 Hz, 1H), 8.07 (br d, J=7.3 Hz, 1H), 7.07-7.13 (m, 1H), 4.13 (s, 3H), 3.99 (br s, 1H), 3.42-3.56 (m, 2H), 3.24 (br d, J=16.1 Hz, 1H), 3.10 (br d, J=15.7 Hz, 1H), 2.91 (br d, J=11.2 Hz, 1H), 2.65 (br d, J=11.2 Hz, 1H), 2.51 (s, 3H), 1.94-2.07 (m, 2H), 1.46 (s, 3H), 1.18 (s, 3H).

### Example 380. (S)—N-(5-(2-(6-(hydroxymethyl)-2,2-dimethylmorpholino)acetamido)-2-methylpyridin-3-yl)-2-(2-methoxypyridin-3-yl)pyrazolo[5,1-b]thiazole-7-carboxamide

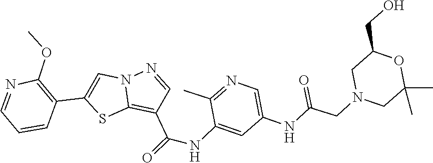 [SMILES: COc1ncccc1-c1cn2ncc(C(=O)Nc3cc(NC(=O)CN4C[C@@H](CO)OC(C)(C)C4)cnc3C)c2s1]

The title compound was prepared from (S)-2-bromo-N-(5-(2-(6-(hydroxymethyl)-2,2-dimethylmorpholino)acetamido)-2-methylpyridin-3-yl)pyrazolo[5,1-b]thiazole-7-carboxamide

according to the procedure of Example 372, substituting 2-methoxypyridine-3-boronic acid for 3-fluoro-5-(4,4,5,5-tetramethyl-1,3,2-dioxaborolan-2-yl)pyridin-2-amine (35 mg, 64%). LCMS (ESI): mass calcd. for C₂₇H₃₁N₇O₅S, 565.7; m/z found, 566.2 [M+H]⁺. ¹H NMR (METHANOL-d4) δ: 8.64 (s, 1H), 8.60 (d, J=2.4 Hz, 1H), 8.43 (s, 1H), 8.26 (d, J=2.0 Hz, 1H), 8.18-8.21 (m, 1H), 8.07 (dd, J=7.6, 1.7 Hz, 1H), 7.10 (dd, J=7.3, 4.9 Hz, 1H), 4.13 (s, 3H), 3.93-4.06 (m, 1H), 3.40-3.58 (m, 3H), 3.24 (d, J=15.7 Hz, 1H), 3.07-3.12 (m, 1H), 2.91 (br d, J=10.8 Hz, 1H), 2.65 (d, J=12.2 Hz, 1H), 2.51 (s, 3H), 1.96-2.06 (m, 2H), 1.46 (s, 1H), 1.18 (s, 3H).

### Example 381. N-(5-(2-(cis-2-(hydroxymethyl)-6-methylmorpholino)acetamido)-2-methylpyridin-3-yl)-2-(2-methoxypyridin-3-yl)pyrazolo[5,1-b]thiazole-7-carboxamide

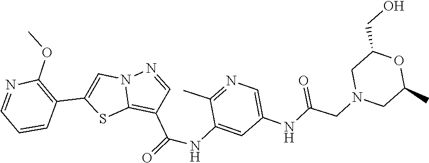 [SMILES: COc1ncccc1-c1cn2ncc(C(=O)Nc3cc(NC(=O)CN4C[C@H](CO)O[C@@H](C)C4)cnc3C)c2s1]

The title compound was prepared from 2-bromo-N-(5-(2-(cis-2-(hydroxymethyl)-6-methylmorpholino)acetamido)-2-methylpyridin-3-yl)pyrazolo[5,1-b]thiazole-7-carboxamide

according to the procedure of Example 372, substituting 2-methoxypyridine-3-boronic acid for 3-fluoro-5-(4,4,5,5-tetramethyl-1,3,2-dioxaborolan-2-yl)pyridin-2-amine (28 mg, 51%). LCMS (ESI): mass calcd. for C₂₆H₂₉N₇O₅S, 551.6; m/z found, 552.3 [M+H]⁺. ¹H NMR (METHANOL-d4) δ: 8.64 (s, 1H), 8.62 (d, J=2.4 Hz, 1H), 8.43 (s, 1H), 8.26 (d, J=2.0 Hz, 1H), 8.19 (dd, J=4.9, 1.5 Hz, 1H), 8.07 (dd, J=7.6, 1.7 Hz, 1H), 7.10 (dd, J=7.3, 4.9 Hz, 1H), 4.13 (s, 3H), 3.72-3.86 (m, 2H), 3.54 (qd, J=11.7, 5.6 Hz, 2H), 3.22 (s, 2H), 2.81-2.94 (m, 2H), 2.51 (s, 3H), 1.94-2.07 (m, 2H), 1.16 (d, J=6.4 Hz, 3H).

### Example 382. 2-(1-cyclopropyl-1H-pyrazol-4-yl)-N-(5-(2-(trans-2,6-dimethylmorpholino)acetamido)-2-methylpyridin-3-yl)pyrazolo[5,1-b]thiazole-7-carboxamide

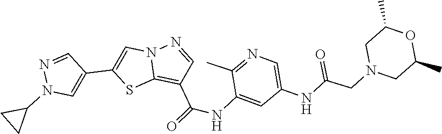 [SMILES: Cc1ncc(NC(=O)CN2C[C@H](C)O[C@@H](C)C2)cc1NC(=O)c1cnn2cc(-c3cnn(C4CC4)c3)sc12]

The title compound was prepared from 2-bromo-N-(5-(2-(trans-2,6-dimethylmorpholino)acetamido)-2-methylpyridin-3-yl)pyrazolo[5,1-b]thiazole-7-carboxamide according to the procedure of Example 372, substituting 1-cyclopropyl-1H-pyrazole-4-boronic acid pinacol ester for 3-fluoro-5-(4,4,5,5-tetramethyl-1,3,2-dioxaborolan-2-yl)pyridin-2-amine (24 mg, 45%). LCMS (ESI): mass calcd. for C₂₆H₃₀N₈O₃S, 534.6; m/z found, 535.2 [M+H]⁺. ¹H NMR (METHANOL-d4) δ: 8.59 (d, J=2.4 Hz, 1H), 8.38 (s, 1H), 8.24 (d, J=2.4 Hz, 1H), 8.22 (s, 1H), 8.12 (s, 1H), 7.81 (s, 1H), 4.06-4.15 (m, 2H), 3.72 (tt, J=7.4, 3.6 Hz, 1H), 3.22 (d, J=15.7 Hz, 1H), 3.04-3.11 (m, 1H), 2.62 (dd, J=11.0, 3.2 Hz, 2H), 2.50 (s, 3H), 2.31 (dd, J=11.0, 5.6 Hz, 2H), 1.29 (d, J=6.4 Hz, 6H), 1.04-1.18 (m, 4H).

### Example 383. N-(5-(2-(6-(methoxymethyl)-2,2-dimethylmorpholino)acetamido)-2-methylpyridin-3-yl)-2-(1-methyl-1H-pyrazol-4-yl)pyrazolo[5,1-b]thiazole-7-carboxamide

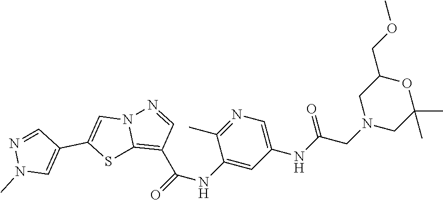

### Step a: 2-bromo-N-(5-(2-(6-(methoxymethyl)-2,2-dimethylmorpholino)acetamido)-2-methylpyridin-3-yl)pyrazolo[5,1-b]thiazole-7-carboxamide

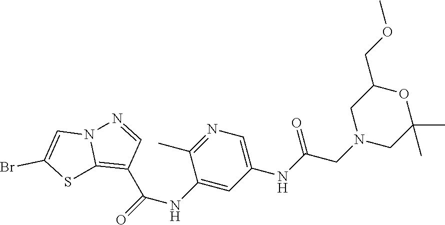 [SMILES: COCC1CN(CC(=O)Nc2cnc(C)c(NC(=O)c3cnn4cc(Br)sc34)c2)CC(C)(C)O1]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with 6-(methoxymethyl)-2,2-dimethylmorpholine (54 mg, 70%). LCMS (ESI): mass calcd. for C₂₂H₂₇BrN₆O₄S, 550.1; m/z found, 550.2/552.2 [M+H]⁺.

### Step b: N-(5-(2-(6-(methoxymethyl)-2,2-dimethylmorpholino)acetamido)-2-methylpyridin-3-yl)-2-(1-methyl-1H-pyrazol-4-yl)pyrazolo[5,1-b]thiazole-7-carboxamide

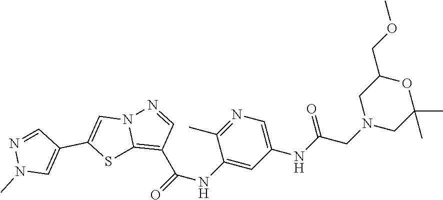

The title compound was prepared from 2-bromo-N-(5-(2-(6-(methoxymethyl)-2,2-dimethylmorpholino)acetamido)-2-methylpyridin-3-yl)pyrazolo[5,1-b]thiazole-7-carboxamide according to the procedure of Example 372, substituting 1-methylpyrazole-4-boronic acid, pinacol ester for 3-fluoro-5-(4,4,5,5-tetramethyl-1,3,2-dioxaborolan-2-yl)pyridin-2-amine (18 mg, 33%). LCMS (ESI): mass calcd. for C₂₆H₃₂N₈O₄S, 552.7; m/z found, 553.3 [M+H]⁺. ¹H NMR (METHANOL-d4) δ: 8.59 (d, J=2.4 Hz, 1H), 8.38 (s, 1H), 8.24 (d, J=2.0 Hz, 1H), 8.22 (s, 1H), 8.03 (s, 1H), 7.82 (s, 1H), 4.12 (dtd, J=10.6, 5.1, 2.7 Hz, 1H), 3.94 (s, 3H), 3.33-3.42 (m, 5H), 3.24 (d, J=15.7 Hz, 1H), 3.06-3.12 (m, 1H), 2.85-2.91 (m, 1H), 2.64 (dd, J=11.2, 1.5 Hz, 1H), 2.50 (s, 3H), 1.95-2.06 (m, 2H), 1.45 (s, 3H), 1.17 (s, 3H).

### Example 384. N-(5-(2-(trans-2,6-dimethylmorpholino)acetamido)-2-methylpyridin-3-yl)-2-(2-methoxypyridin-3-yl)pyrazolo[5,1-b]thiazole-7-carboxamide

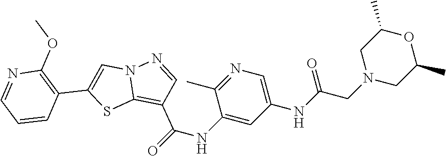 [SMILES: COc1ncccc1-c1cn2ncc(C(=O)Nc3cc(NC(=O)CN4C[C@H](C)O[C@@H](C)C4)cnc3C)c2s1]

The title compound was prepared from 2-bromo-N-(5-(2-(trans-2,6-dimethylmorpholino)acetamido)-2-methylpyridin-3-yl)pyrazolo[5,1-b]thiazole-7-carboxamide according to the procedure of Example 372, substituting 3-methoxypyridine-4-boronic acid for 3-fluoro-5-(4,4,5,5-tetramethyl-1,3,2-dioxaborolan-2-yl)pyridin-2-amine (26 mg, 48%). LCMS (ESI): mass calcd. for C₂₆H₂₉N₇O₄S, 535.6; m/z found, 536.2 [M+H]⁺. ¹H NMR (METHANOL-d4) δ: 8.86 (s, 1H), 8.59 (d, 1H, J=2.4 Hz), 8.48 (d, 2H, J=3.4 Hz), 8.2-8.3 (m, 2H), 7.75 (d, 1H, J=5.4 Hz), 4.16 (s, 3H), 4.10 (ttd, 2H, J=3.5, 6.3, 9.4 Hz), 3.2 (m, 1H), 3.0-3.1 (m, 1H), 2.63 (dd, 2H, J=3.2, 11.0 Hz), 2.51 (s, 3H), 2.32 (dd, 2H, J=5.9, 10.8), 1.29 (d, 6H, J=6.8 Hz).

### Example 385. N-(5-(2-(trans-2,6-dimethylmorpholino)acetamido)-2-methylpyridin-3-yl)-2-(4-methoxypyridin-3-yl)pyrazolo[5,1-b]thiazole-7-carboxamide

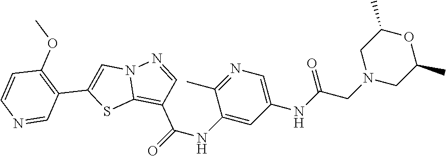 [SMILES: COc1ccncc1-c1cn2ncc(C(=O)Nc3cc(NC(=O)CN4C[C@H](C)O[C@@H](C)C4)cnc3C)c2s1]

The title compound was prepared from 2-bromo-N-(5-(2-(trans-2,6-dimethylmorpholino)acetamido)-2-methylpyridin-3-yl)pyrazolo[5,1-b]thiazole-7-carboxamide according to the procedure of Example 372, substituting 4-methoxypyridine-3-boronic acid hydrate for 3-fluoro-5-(4,4,5,5-tetramethyl-1,3,2-dioxaborolan-2-yl)pyridin-2-amine (16 mg, 27%). LCMS (ESI): mass calcd. for C₂₆H₂₉N₇O₄S, 535.6; m/z found, 536.2 [M+H]⁺. ¹H NMR (METHANOL-d4) δ: 8.76 (s, 1H), 8.67 (s, 1H), 8.59 (s, 1H), 8.45 (s, 2H), 8.24-8.27 (m, 1H), 7.27 (d, J=5.9 Hz, 1H), 4.06-4.14 (m, 5H), 3.19-3.25 (m, 1H), 3.04-3.11 (m, 1H), 2.61 (br s, 2H), 2.51 (s, 3H), 2.29-2.36 (m, 2H), 1.29 (d, J=6.8 Hz, 6H).

### Example 386. N-(5-(2-(trans-2,6-dimethylmorpholino)acetamido)-2-methylpyridin-3-yl)-2-(6-methoxypyridin-2-yl)pyrazolo[5,1-b]thiazole-7-carboxamide

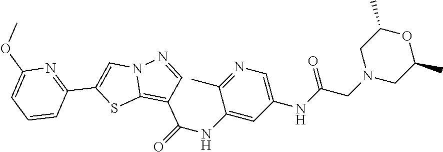 [SMILES: COc1cccc(-c2cn3ncc(C(=O)Nc4cc(NC(=O)CN5C[C@H](C)O[C@@H](C)C5)cnc4C)c3s2)n1]

The title compound was prepared from 2-bromo-N-(5-(2-(trans-2,6-dimethylmorpholino)acetamido)-2-methylpyridin-3-yl)pyrazolo[5,1-b]thiazole-7-carboxamide according to the procedure of Example 372, substituting 6-methoxypyridine-2-boronic acid for 3-fluoro-5-(4,4,5,5-tetramethyl-1,3,2-dioxaborolan-2-yl)pyridin-2-amine (20 mg, 37%). LCMS (ESI): mass calcd. for C₂₆H₂₉N₇O₄S, 535.6; m/z found, 536.2 [M+H]⁺. ¹H NMR (METHANOL-d4) δ: 8.74 (s, 1H), 8.60 (d, J=2.0 Hz, 1H), 8.44 (s, 1H), 8.25 (d, J=2.0 Hz, 1H), 7.74 (t, J=8.1 Hz, 1H), 7.51 (d, J=7.3 Hz, 1H), 6.76 (d, J=8.3 Hz, 1H), 4.05-4.15 (m, 2H), 3.99 (s, 3H), 3.19-3.25 (m, 1H), 3.04-3.11 (m, 1H), 2.63 (dd, J=10.8, 2.9 Hz, 2H), 2.51 (s, 3H), 2.32 (dd, J=11.0, 5.6 Hz, 2H), 1.29 (d, J=6.4 Hz, 6H).

### Example 387. N-(5-((2-(3,3-dimethylazetidin-1-yl)ethyl)carbamoyl)-2-methylpyridin-3-yl)-2-(2-methoxypyridin-3-yl)pyrazolo[5,1-b]thiazole-7-carboxamide

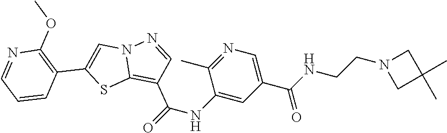 [SMILES: COc1ncccc1-c1cn2ncc(C(=O)Nc3cc(C(=O)NCCN4CC(C)(C)C4)cnc3C)c2s1]

The title compound was prepared from 2-bromo-N-(5-((2-(3,3-dimethylazetidin-1-yl)ethyl)carbamoyl)-2-methylpyridin-3-yl)pyrazolo[5,1-b]thiazole-7-carboxamide according to the procedure of Example 372, substituting 2-methoxypyridine-3-boronic acid for 3-fluoro-5-(4,4,5,5-tetramethyl-1,3,2-dioxaborolan-2-yl)pyridin-2-amine (8 mg, 15%). LCMS (ESI): mass calcd. for C₂₆H₂₉N₇O₃S, 519.6; m/z found, 520.2 [M+H]⁺. ¹H NMR (METHANOL-d4) δ: 8.78 (d, J=2.0 Hz, 1H), 8.65 (s, 1H), 8.45 (s, 1H), 8.31 (d, J=2.0 Hz, 1H), 8.19 (dd, J=4.9, 1.5 Hz, 1H), 8.07 (dd, J=7.6, 1.7 Hz, 1H), 7.10 (dd, J=7.8, 4.9 Hz, 1H), 4.13 (s, 3H), 3.39-3.45 (m, 2H), 3.09-3.17 (m, 6H), 2.75 (t, J=6.6 Hz, 2H), 2.61 (s, 3H), 1.24 (s, 6H).

### Example 388. N-(5-(2-(3,3-dimethylazetidin-1-yl)acetamido)-2-methylpyridin-3-yl)-2-(1-ethyl-1H-pyrazol-5-yl)pyrazolo[5,1-b]thiazole-7-carboxamide

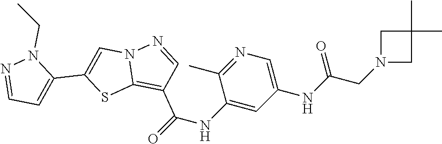 [SMILES: CCn1nccc1-c1cn2ncc(C(=O)Nc3cc(NC(=O)CN4CC(C)(C)C4)cnc3C)c2s1]

The title compound was prepared from 2-bromo-N-(5-(2-(3,3-dimethylazetidin-1-yl)acetamido)-2-methylpyridin-3-yl)pyrazolo[5,1-b]thiazole-7-carboxamide according to Example 370 Step b, substituting 1-ethylpyrazole-5-boronic acid pinacol ester for (2-oxo-1,2-dihydropyridin-3-yl)boronic acid (9 mg, 14%). LCMS (ESI): mass calcd. for C₂₄H₂₈N₈O₂S, 492.6; m/z found, 493.1 [M+H]⁺. ¹H NMR (METHANOL-d4) δ: 8.61-8.65 (m, 1H), 8.49 (s, 1H), 8.41 (s, 1H), 8.32-8.37 (m, 1H), 7.60 (d, J=2.0 Hz, 1H), 6.61 (d, J=2.0 Hz, 1H), 4.36 (q, J=7.3 Hz, 2H), 4.30 (s, 2H), 4.07-4.16 (m, 2H), 3.94-4.04 (m, 2H), 2.56 (s, 3H), 1.45 (t, J=7.3 Hz, 5H), 1.33-1.48 (m, 1H), 1.35 (br s, 3H).

### Example 389. 2-(1,4-dimethyl-1H-pyrazol-5-yl)-N-(5-(2-(3,3-dimethylazetidin-1-yl)acetamido)-2-methylpyridin-3-yl)pyrazolo[5,1-b]thiazole-7-carboxamide

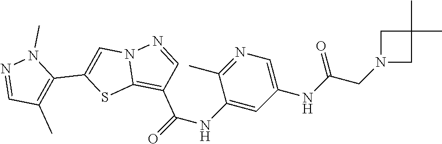 [SMILES: Cc1cnn(C)c1-c1cn2ncc(C(=O)Nc3cc(NC(=O)CN4CC(C)(C)C4)cnc3C)c2s1]

The title compound was prepared from 2-bromo-N-(5-(2-(3,3-dimethylazetidin-1-yl)acetamido)-2-methylpyridin-3-yl)pyrazolo[5,1-b]thiazole-7-carboxamide according to Example 370 Step b, substituting 1,4-dimethyl-5-(4,4,5,5-tetramethyl-1,3,2-dioxaborolan-2-yl)-1H-pyrazole for (2-oxo-1,2-dihydropyridin-3-yl)boronic acid (9 mg, 14%). LCMS (ESI): mass calcd. for C₂₄H₂₈N₈O₂S, 492.6; m/z found, 493.1 [M+H]⁺. ¹H NMR (METHANOL-d4) δ: 8.56 (d, J=2.4 Hz, 1H), 8.48 (s, 1H), 8.34 (s, 1H), 8.23 (d, J=2.0 Hz, 1H), 7.42 (s, 1H), 3.90 (s, 3H), 3.36 (s, 2H), 3.20 (s, 4H), 2.50 (s, 3H), 2.14 (s, 3H), 1.26 (s, 6H).

### Example 390. N-(5-(2-(3,3-dimethylazetidin-1-yl)acetamido)-2-methylpyridin-3-yl)-2-(2-methylpyridin-3-yl)pyrazolo[5,1-b]thiazole-7-carboxamide

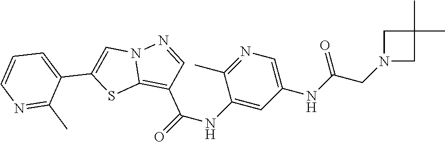 [SMILES: Cc1ncc(NC(=O)CN2CC(C)(C)C2)cc1NC(=O)c1cnn2cc(-c3cccnc3C)sc12]

The title compound was prepared from 2-bromo-N-(5-((2-(3,3-dimethylazetidin-1-yl)ethyl)carbamoyl)-2-methylpyridin-3-yl)pyrazolo[5,1-b]thiazole-7-carboxamide according to the procedure of Example 372, substituting 2-methylpyridine-3-boronic acid for 3-fluoro-5-(4,4,5,5-tetramethyl-1,3,2-dioxaborolan-2-yl)pyridin-2-amine (15 mg, 24%). LCMS (ESI): mass calcd. for C₂₅H₂₇N₅O₂S, 489.6; m/z found, 490.1 [M+H]⁺. ¹H NMR (METHANOL-d4) δ: 8.70 (s, 1H), 8.66 (d, J=4.9 Hz, 1H), 8.51 (s, 1H), 8.43 (s, 1H), 8.40 (s, 1H), 8.29 (br d, J=6.8 Hz, 1H), 7.66-7.73 (m, 1H), 4.32 (s, 2H), 3.92-4.19 (m, 4H), 2.81 (s, 3H), 2.59 (s, 3H), 1.31-1.51 (m, 6H).

### Example 391. N-(5-(2-(3,3-dimethylazetidin-1-yl)acetamido)-2-methylpyridin-3-yl)-2-(2-ethylpyridin-3-yl)pyrazolo[5,1-b]thiazole-7-carboxamide

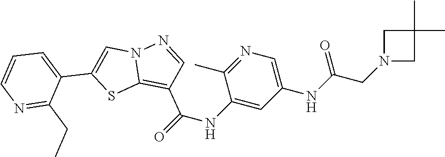 [SMILES: CCc1ncccc1-c1cn2ncc(C(=O)Nc3cc(NC(=O)CN4CC(C)(C)C4)cnc3C)c2s1]

The title compound was prepared from 2-bromo-N-(5-((2-(3,3-dimethylazetidin-1-yl)ethyl)carbamoyl)-2-methylpyridin-3-yl)pyrazolo[5,1-b]thiazole-7-carboxamide according to the procedure of Example 372, substituting 2-ethylpyridine-3-boronic acid for 3-fluoro-5-(4,4,5,5-tetramethyl-1,3,2-dioxaborolan-2-yl)pyridin-2-amine (15 mg, 24%). LCMS (ESI): mass calcd. for C₂₆H₂₉N₅O₂S, 503.6; m/z found, 504.3 [M+H]⁺. ¹H NMR (METHANOL-d4) δ: 8.76 (d, J=2.0 Hz, 1H), 8.71 (d, J=5.4 Hz, 1H), 8.52 (s, 1H), 8.48 (d, J=2.0 Hz, 1H), 8.39 (s, 1H), 8.27 (d, J=7.8 Hz, 1H), 8.10-8.13 (m, 1H), 7.69 (dd, J=7.8, 5.4 Hz, 1H), 7.42-7.45 (m, 1H), 4.34 (s, 2H), 3.94-4.20 (m, 4H), 3.10 (q, J=7.8 Hz, 2H), 2.62 (s, 3H), 1.31-1.51 (m, 9H).

### Example 392. 2-(2,4-dimethoxypyrimidin-5-yl)-N-(5-(2-(3,3-dimethylazetidin-1-yl)acetamido)-2-methylpyridin-3-yl)pyrazolo[5,1-b]thiazole-7-carboxamide

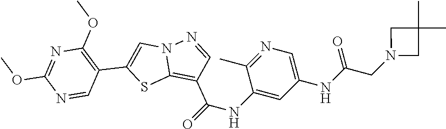

The title compound was prepared from 2-bromo-N-(5-((2-(3,3-dimethylazetidin-1-yl)ethyl)carbamoyl)-2-methylpyridin-3-yl)pyrazolo[5,1-b]thiazole-7-carboxamide according to the procedure of Example 372, substituting 2,4-dimethoxypyrimidine-5-boronic acid for 3-fluoro-5-(4,4,5,5-tetramethyl-1,3,2-dioxaborolan-2-yl)pyridin-2-amine (8 mg, 14%). LCMS (ESI): mass calcd. for C₂₅H₂₈N₈O₄S, 536.6; m/z found, 537.1 [M+H]⁺. ¹H NMR (METHANOL-d4) δ: 8.62-8.64 (m, 1H), 8.56 (d, J=2.4 Hz, 1H), 8.53 (s, 1H), 8.43 (s, 1H), 8.24 (d, J=2.0 Hz, 1H), 4.19 (s, 3H), 4.12 (s, 2H), 4.06 (s, 3H), 3.89 (s, 4H), 2.52 (s, 3H), 1.38 (s, 6H).

### Example 393. 2-(3,6-dimethoxypyridazin-4-yl)-N-(5-(2-(3,3-dimethylazetidin-1-1)acetamido)-2-methylpyridin-3-yl)pyrazolo[5,1-b]thiazole-7-carboxamide

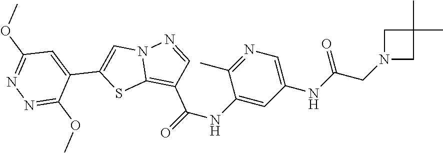 [SMILES: COc1cc(-c2cn3ncc(C(=O)Nc4cc(NC(=O)CN5CC(C)(C)C5)cnc4C)c3s2)c(OC)nn1]

The title compound was prepared from 2-bromo-N-(5-((2-(3,3-dimethylazetidin-1-yl)ethyl)carbamoyl)-2-methylpyridin-3-yl)pyrazolo[5,1-b]thiazole-7-carboxamide according to the procedure of Example 372, substituting 3,6-dimethoxylpyridazine-4-boronic acid for 3-fluoro-5-(4,4,5,5-tetramethyl-1,3,2-dioxaborolan-2-yl)pyridin-2-amine (6 mg, 9%). LCMS (ESI): mass calcd. for C₂₅H₂₈N₈O₄S, 536.6; m/z found, 537.3 [M+H]⁺. ¹H NMR (METHANOL-d4) δ: 8.62-8.64 (m, 1H), 8.56 (d, J=2.4 Hz, 1H), 8.53 (s, 1H), 8.43 (s, 1H), 8.24 (d, J=2.0 Hz, 1H), 4.19 (s, 3H), 4.12 (s, 2H), 4.06 (s, 3H), 3.89 (s, 4H), 2.52 (s, 3H), 1.38 (s, 6H).

### Example 394. N-(5-(2-(3,3-dimethylazetidin-1-yl)acetamido)-2-methylpyridin-3-yl)-2-(6-methoxypyridin-2-yl)pyrazolo[5,1-b]thiazole-7-carboxamide

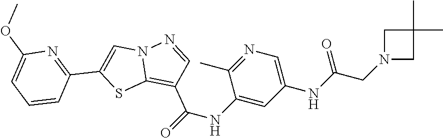 [SMILES: COc1cccc(-c2cn3ncc(C(=O)Nc4cc(NC(=O)CN5CC(C)(C)C5)cnc4C)c3s2)n1]

The title compound was prepared from 2-bromo-N-(5-((2-(3,3-dimethylazetidin-1-yl)ethyl)carbamoyl)-2-methylpyridin-3-yl)pyrazolo[5,1-b]thiazole-7-carboxamide according to the procedure of Example 372, substituting 6-methoxypyridine-2-boronic acid for 3-fluoro-5-(4,4,5,5-tetramethyl-1,3,2-dioxaborolan-2-yl)pyridin-2-amine (12 mg, 23%). LCMS (ESI): mass calcd. for C₂₅H₂₇N₇O₃S, 505.6; m/z found, 506.2 [M+H]⁺. ¹H NMR (METHANOL-d4) δ: 8.77 (s, 1H), 8.71 (br s, 1H), 8.46 (s, 1H), 8.43 (br s, 1H), 7.72-7.78 (m, 1H), 7.52 (d, J=7.3 Hz, 1H), 6.78 (d, J=8.3 Hz, 1H), 4.33 (s, 2H), 3.95-4.17 (m, 7H), 2.60 (d, J=1.0 Hz, 3H), 1.31-1.51 (m, 6H).

### Example 395. N-(5-(2-(3,3-dimethylazetidin-1-yl)acetamido)-2-methylpyridin-3-yl)-2-(6-methoxypyridin-3-yl)pyrazolo[5,1-b]thiazole-7-carboxamide

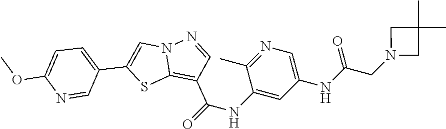

The title compound was prepared from 2-bromo-N-(5-((2-(3,3-dimethylazetidin-1-yl)ethyl)carbamoyl)-2-methylpyridin-3-yl)pyrazolo[5,1-b]thiazole-7-carboxamide according to the procedure of Example 372, substituting 2-methoxy-5-pyridineboronic acid for 3-fluoro-5-(4,4,5,5-tetramethyl-1,3,2-dioxaborolan-2-yl)pyridin-2-amine (3.5 mg, 6%). LCMS (ESI): mass calcd. for C₂₅H₂₇N₇O₃S, 505.6; m/z found, 506.1 [M+H]⁺. ¹H NMR (METHANOL-d4) δ: 8.57 (d, J=2.4 Hz, 1H), 8.45-8.48 (m, 2H), 8.42 (s, 1H), 8.22 (d, J=2.4 Hz, 1H), 7.99 (dd, J=8.6, 2.7 Hz, 1H), 6.91 (d, J=8.8 Hz, 1H), 3.97 (s, 1H), 3.96-3.98 (m, 1H), 3.39-3.39 (m, 1H), 3.39 (s, 1H), 3.23 (s, 3H), 3.22-3.24 (m, 1H), 2.50 (s, 3H), 1.27 (s, 6H).

### Example 396. N-(5-(2-(3-methoxy-3-methylazetidin-1-yl)acetamido)-2-methylpyridin-3-yl)-2-(2-methoxypyridin-3-yl)pyrazolo[5,1-b]thiazole-7-carboxamide

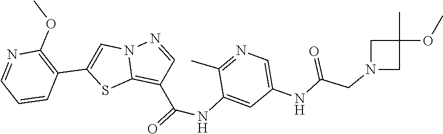 [SMILES: COc1ncccc1-c1cn2ncc(C(=O)Nc3cc(NC(=O)CN4CC(C)(OC)C4)cnc3C)c2s1]

### Step a: 2-bromo-N-(5-(2-(3-methoxy-3-methylazetidin-1-yl)acetamido)-2-methylpyridin-3-yl)pyrazolo[5,1-b]thiazole-7-carboxamide

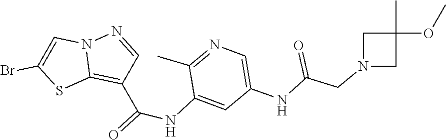 [SMILES: COC1(C)CN(CC(=O)Nc2cnc(C)c(NC(=O)c3cnn4cc(Br)sc34)c2)C1]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with 3-methoxy-3-methylazetidine hydrochloride (97 mg, 55%). LCMS (ESI): mass calcd. for C₁₉H₂₁BrN₆O₃S, 493.4; m/z found, 493.0/495.0 [M+H]⁺.

### Step b: N-(5-(2-(3-methoxy-3-methylazetidin-1-yl)acetamido)-2-methylpyridin-3-yl)-2-(2-methoxypyridin-3-yl)pyrazolo[5,1-b]thiazole-7-carboxamide

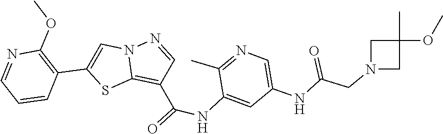 [SMILES: COc1ncccc1-c1cn2ncc(C(=O)Nc3cc(NC(=O)CN4CC(C)(OC)C4)cnc3C)c2s1]

The title compound was prepared from 2-bromo-N-(5-(2-(3-methoxy-3-methylazetidin-1-yl)acetamido)-2-methylpyridin-3-yl)pyrazolo[5,1-b]thiazole-7-carboxamide according to the procedure of Example 372, substituting 2-methoxypyridine-3-boronic acid for 3-fluoro-5-(4,4,5,5-tetramethyl-1,3,2-dioxaborolan-2-yl)pyridin-2-amine (29 mg, 47%). LCMS (ESI): mass calcd. for C₂₅H₂₇N₇O₄S, 521.6; m/z found, 522.3 [M+H]⁺. ¹H NMR (DMSO-d6) δ: 10.75 (br s, 1H), 9.89 (br s, 1H), 9.00 (s, 1H), 8.60 (s, 1H), 8.53 (br d, J=7.3 Hz, 1H), 8.18-8.28 (m, 3H), 7.18 (dd, J=7.3, 4.9 Hz, 1H), 4.34 (br d, J=5.4 Hz, 2H), 4.16 (br s, 3H), 4.07 (s, 3H), 4.04-4.23 (m, 1H), 3.21 (br d, J=12.7 Hz, 3H), 2.46 (s, 3H), 1.39-1.54 (m, 3H).

### Example 397. N-(5-(2-(azetidin-1-yl)acetamido)-2-methylpyridin-3-yl)-2-(2-methoxypyridin-3-yl)pyrazolo[5,1-b]thiazole-7-carboxamide

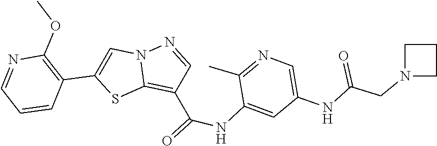 [SMILES: COc1ncccc1-c1cn2ncc(C(=O)Nc3cc(NC(=O)CN4CCC4)cnc3C)c2s1]

### Step a: N-(5-(2-(azetidin-1-yl)acetamido)-2-methylpyridin-3-yl)-2-bromopyrazolo[5,1-b]thiazole-7-carboxamide

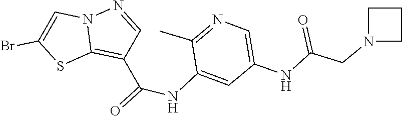 [SMILES: Cc1ncc(NC(=O)CN2CCC2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with azetidine (76 mg, 44%). LCMS (ESI): mass calcd. for C₁₇H₁₇BrN₆O₂S, 449.3; m/z found, 449.0/451.0 [M+H]⁺.

### Step b: N-(5-(2-(azetidin-1-yl)acetamido)-2-methylpyridin-3-yl)-2-(2-methoxypyridin-3-yl)pyrazolo[5,1-b]thiazole-7-carboxamide

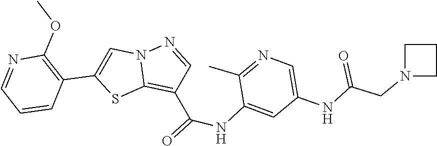 [SMILES: COc1ncccc1-c1cn2ncc(C(=O)Nc3cc(NC(=O)CN4CCC4)cnc3C)c2s1]

The title compound was prepared from N-(5-(2-(azetidin-1-yl)acetamido)-2-methylpyridin-3-yl)-2-bromopyrazolo[5,1-b]thiazole-7-carboxamide according to the procedure of Example 372, substituting 2-methoxypyridine-3-boronic acid for 3-fluoro-5-(4,4,5,5-tetramethyl-1,3,2-dioxaborolan-2-yl)pyridin-2-amine (24 mg, 23%). LCMS (ESI): mass calcd. for C₂₃H₂₃N₇O₃S, 477.5; m/z found, 478.1 [M+H]⁺. ¹H NMR (METHANOL-d4) δ: 8.69 (br d, J=2.0 Hz, 1H), 8.67 (s, 1H), 8.45 (s, 1H), 8.39 (br s, 1H), 8.20 (dd, J=4.9, 1.5 Hz, 1H), 8.08 (dd, J=7.6, 1.7 Hz, 1H), 7.11 (dd, J=7.3, 4.9 Hz, 1H), 4.17-4.45 (m, 5H), 4.13 (s, 3H), 2.45-2.72 (m, 5H).

### Example 398. N-(5-(2-(3,3-dimethylazetidin-1-yl)acetamido)-2-methylpyridin-3-yl)-2-(4-methylpyrimidin-5-yl)pyrazolo[5,1-b]thiazole-7-carboxamide

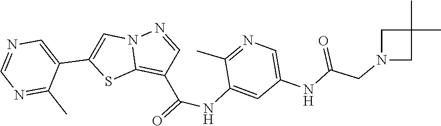 [SMILES: Cc1ncc(NC(=O)CN2CC(C)(C)C2)cc1NC(=O)c1cnn2cc(-c3cncnc3C)sc12]

The title compound was prepared from 2-bromo-N-(5-((2-(3,3-dimethylazetidin-1-yl)ethyl)carbamoyl)-2-methylpyridin-3-yl)pyrazolo[5,1-b]thiazole-7-carboxamide according to the procedure of Example 372, substituting 4-methyl-5-(4,4,5,5-tetramethyl-1,3,2-dioxaborolan-2-yl)pyrimidine for 3-fluoro-5-(4,4,5,5-tetramethyl-1,3,2-dioxaborolan-2-yl)pyridin-2-amine (10 mg, 16%). LCMS (ESI): mass calcd. for C₂₄H₂₆N₈O₂S, 490.6; m/z found, 491.1 [M+H]⁺. ¹H NMR (METHANOL-d4) δ: 9.08 (s, 1H), 8.83 (s, 1H), 8.70 (br d, J=2.0 Hz, 1H), 8.51 (s, 1H), 8.40-8.48 (m, 2H), 4.32 (s, 2H), 3.93-4.17 (m, 4H), 2.73 (s, 3H), 2.59 (s, 3H), 1.32-1.52 (m, 6H).

### Example 399. N-(5-(2-(3,3-dimethylazetidin-1-yl)acetamido)-2-methylpyridin-3-yl)-2-(3-methoxypyridin-4-yl)pyrazolo[5,1-b]thiazole-7-carboxamide

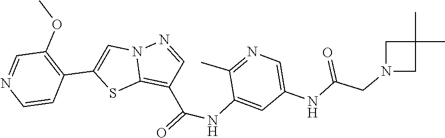 [SMILES: COc1cnccc1-c1cn2ncc(C(=O)Nc3cc(NC(=O)CN4CC(C)(C)C4)cnc3C)c2s1]

The title compound was prepared from 2-bromo-N-(5-((2-(3,3-dimethylazetidin-1-yl)ethyl)carbamoyl)-2-methylpyridin-3-yl)pyrazolo[5,1-b]thiazole-7-carboxamide according to the procedure of Example 372, substituting 3-methoxypyridine-4-boronic acid for 3-fluoro-5-(4,4,5,5-tetramethyl-1,3,2-dioxaborolan-2-yl)pyridin-2-amine (29 mg, 24%). LCMS (ESI): mass calcd. for C₂₅H₂₇N₇O₃S, 505.6; m/z found, 506.2 [M+H]⁺. ¹H NMR (METHANOL-d4) δ: 9.05 (s, 1H), 8.69 (d, J=2.4 Hz, 1H), 8.61 (s, 1H), 8.54 (s, 1H), 8.36-8.42 (m, 2H), 8.02 (d, J=5.4 Hz, 1H), 4.33 (s, 2H), 4.22 (s, 3H), 3.93-4.17 (m, 4H), 2.59 (s, 3H), 1.31-1.54 (m, 6H).

### Example 400. N-(5-(2-(3-methoxyazetidin-1-yl)acetamido)-2-methylpyridin-3-yl)-2-(2-methoxypyridin-3-yl)pyrazolo[5,1-b]thiazole-7-carboxamide

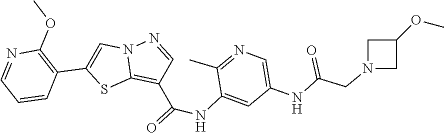 [SMILES: COc1ncccc1-c1cn2ncc(C(=O)Nc3cc(NC(=O)CN4CC(OC)C4)cnc3C)c2s1]

### Step a: 2-bromo-N-(5-(2-(3-methoxyazetidin-1-yl)acetamido)-2-methylpyridin-3-yl)pyrazolo[5,1-b]thiazole-7-carboxamide

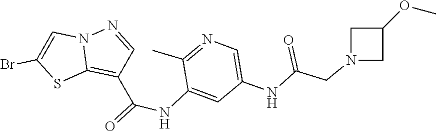 [SMILES: COC1CN(CC(=O)Nc2cnc(C)c(NC(=O)c3cnn4cc(Br)sc34)c2)C1]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with 3-methoxyazetidine hydrochloride (114 mg, 64%). LCMS (ESI): mass calcd. for C₁₈H₁₉BrN₆O₃S, 479.4; m/z found, 479.0/481.0 [M+H]⁺.

### Step b: N-(5-(2-(azetidin-1-yl)acetamido)-2-methylpyridin-3-yl)-2-(2-methoxypyridin-3-yl)pyrazolo[5,1-b]thiazole-7-carboxamide

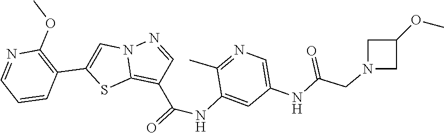 [SMILES: COc1ncccc1-c1cn2ncc(C(=O)Nc3cc(NC(=O)CN4CC(OC)C4)cnc3C)c2s1]

The title compound was prepared from 2-bromo-N-(5-(2-(3-methoxyazetidin-1-yl)acetamido)-2-methylpyridin-3-yl)pyrazolo[5,1-b]thiazole-7-carboxamide according to the procedure of Example 372, substituting 2-methoxypyridine-3-boronic acid for 3-fluoro-5-(4,4,5,5-tetramethyl-1,3,2-dioxaborolan-2-yl)pyridin-2-amine (24 mg, 23%). LCMS (ESI): mass calcd. for C₂₄H₂₅N₇O₄S, 507.6; m/z found, 508.1 [M+H]⁺. ¹H NMR (METHANOL-d4) δ: 8.72 (d, J=2.0 Hz, 1H), 8.67 (s, 1H), 8.46 (s, 1H), 8.43 (d, J=2.0 Hz, 1H), 8.20 (dd, J=4.9, 1.5 Hz, 1H), 8.08 (dd, J=7.8, 1.5 Hz, 1H), 7.11 (dd, J=7.6, 5.1 Hz, 1H), 4.32-4.38 (m, 3H), 4.31-4.72 (m, 1H), 4.13 (s, 3H), 4.11-4.28 (m, 1H), 3.37 (s, 3H), 2.60 (s, 3H).

### Example 401. N-(5-(2-(3-methoxyazetidin-1-yl)acetamido)-2-methylpyridin-3-yl)-2-(2-ethoxypyridin-3-yl)pyrazolo[5,1-b]thiazole-7-carboxamide

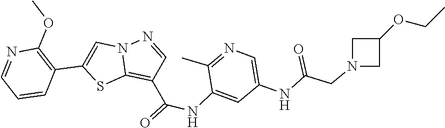 [SMILES: CCOC1CN(CC(=O)Nc2cnc(C)c(NC(=O)c3cnn4cc(-c5cccnc5OC)sc34)c2)C1]

### Step a: 2-bromo-N-(5-(2-(3-ethoxyazetidin-1-yl)acetamido)-2-methylpyridin-3-yl)pyrazolo[5,1-b]thiazole-7-carboxamide

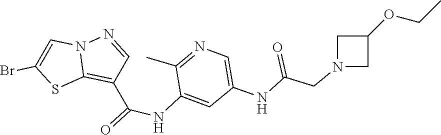 [SMILES: CCOC1CN(CC(=O)Nc2cnc(C)c(NC(=O)c3cnn4cc(Br)sc34)c2)C1]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with 3-ethoxyazetidine hydrochloride (103 mg, 54%). LCMS (ESI): mass calcd. for C₁₉H₂₁BrN₆O₃S, 493.4; m/z found, 493.0/495.0 [M+H]⁺.

### Step b: N-(5-(2-(azetidin-1-yl)acetamido)-2-methylpyridin-3-yl)-2-(2-ethoxypyridin-3-yl)pyrazolo[5,1-b]thiazole-7-carboxamide

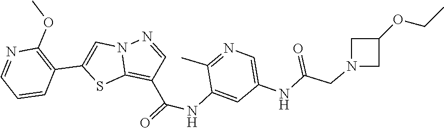 [SMILES: CCOC1CN(CC(=O)Nc2cnc(C)c(NC(=O)c3cnn4cc(-c5cccnc5OC)sc34)c2)C1]

The title compound was prepared from 2-bromo-N-(5-(2-(3-ethoxyazetidin-1-yl)acetamido)-2-methylpyridin-3-yl)pyrazolo[5,1-b]thiazole-7-carboxamide according to the procedure of Example 372, substituting 2-methoxypyridine-3-boronic acid for 3-fluoro-5-(4,4,5,5-tetramethyl-1,3,2-dioxaborolan-2-yl)pyridin-2-amine (33 mg, 51%). LCMS (ESI): mass calcd. for C₂₅H₂₇N₇O₄S, 521.6; m/z found, 522.3 [M+H]⁺. ¹H NMR (METHANOL-d4) δ: 8.71 (d, J=2.4 Hz, 1H), 8.67 (s, 1H), 8.46 (s, 1H), 8.42 (d, J=2.4 Hz, 1H), 8.20 (dd, J=4.9, 2.0 Hz, 1H), 8.08 (dd, J=7.3, 1.5 Hz, 1H), 7.11 (dd, J=7.8, 4.9 Hz, 1H), 4.09-4.73 (m, 10H), 3.54 (q, J=6.8 Hz, 2H), 2.60 (s, 3H), 1.23 (t, J=6.8 Hz, 3H).

### Example 402. N-(5-(2-(3-fluoroazetidin-1-yl)acetamido)-2-methylpyridin-3-yl)-2-(2-methoxypyridin-3-yl)pyrazolo[5,1-b]thiazole-7-carboxamide

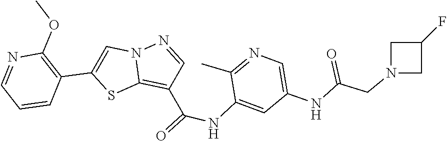 [SMILES: COc1ncccc1-c1cn2ncc(C(=O)Nc3cc(NC(=O)CN4CC(F)C4)cnc3C)c2s1]

### Step a: 2-bromo-N-(5-(2-(3-fluoroazetidin-1-yl)acetamido)-2-methylpyridin-3-yl)pyrazolo[5,1-b]thiazole-7-carboxamide

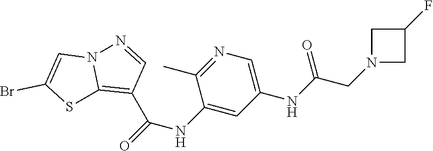 [SMILES: Cc1ncc(NC(=O)CN2CC(F)C2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with 3-fluoroazetidine hydrochloride (100 mg, 73%). LCMS (ESI): mass calcd. for C₁₇H₁₆BrFN₆O₂S, 467.3; m/z found, 467.0/469.0 [M+H]⁺.

### Step b: N-(5-(2-(3-fluoroazetidin-1-yl)acetamido)-2-methylpyridin-3-yl)-2-(2-methoxypyridin-3-yl)pyrazolo[5,1-b]thiazole-7-carboxamide

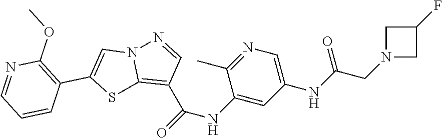 [SMILES: COc1ncccc1-c1cn2ncc(C(=O)Nc3cc(NC(=O)CN4CC(F)C4)cnc3C)c2s1]

The title compound was prepared from 2-bromo-N-(5-(2-(3-fluoroazetidin-1-yl)acetamido)-2-methylpyridin-3-yl)pyrazolo[5,1-b]thiazole-7-carboxamide according to the procedure of Example 372, substituting 2-methoxypyridine-3-boronic acid for 3-fluoro-5-(4,4,5,5-tetramethyl-1,3,2-dioxaborolan-2-yl)pyridin-2-amine (31 mg, 48%). LCMS (ESI): mass calcd. for C₂₃H₂₂FN₇O₃S, 495.5; m/z found, 496.1 [M+H]⁺. ¹H NMR (METHANOL-d4) δ: 8.71-8.75 (m, 1H), 8.67 (s, 1H), 8.46 (s, 1H), 8.44 (d, J=2.4 Hz, 1H), 8.20 (dd, J=4.9, 1.5 Hz, 1H), 8.08 (dd, J=7.6, 1.7 Hz, 1H), 7.11 (dd, J=7.3, 4.9 Hz, 1H), 5.34-5.57 (m, 1H), 4.45-4.76 (m, 4H), 4.42 (s, 2H), 4.13 (s, 3H), 2.60 (s, 3H).

### Example 403. N-(5-(2-(7-oxa-2-azaspiro[3.5]nonan-2-yl)acetamido)-2-methylpyridin-3-yl)-2-(2-methoxypyridin-3-yl)pyrazolo[5,1-b]thiazole-7-carboxamide carboxamide

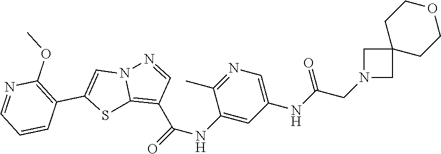 [SMILES: COc1ncccc1-c1cn2ncc(C(=O)Nc3cc(NC(=O)CN4CC5(CCOCC5)C4)cnc3C)c2s1]

### Step a: N-(5-(2-(7-oxa-2-azaspiro[3.5]nonan-2-yl)acetamido)-2-methylpyridin-3-yl)-2-bromopyrazolo[5,1-b]thiazole-7-carboxamide

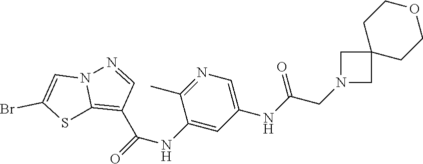 [SMILES: Cc1ncc(NC(=O)CN2CC3(CCOCC3)C2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with 7-oxa-2-azaspiro[3.5]nonane (133 mg, 73%). LCMS (ESI): mass calcd. for C₂₁H₂₃BrN₆O₃S, 519.4; m/z found, 519.1/521.1 [M+H]⁺.

### Step b: N-(5-(2-(7-oxa-2-azaspiro[3.5]nonan-2-yl)acetamido)-2-methylpyridin-3-yl)-2-(2-methoxypyridin-3-yl)pyrazolo[5,1-b]thiazole-7-carboxamide carboxamide

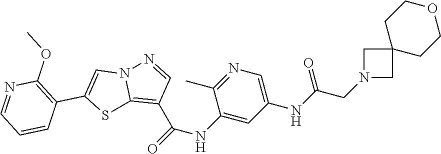 [SMILES: COc1ncccc1-c1cn2ncc(C(=O)Nc3cc(NC(=O)CN4CC5(CCOCC5)C4)cnc3C)c2s1]

The title compound was prepared from N-(5-(2-(7-oxa-2-azaspiro[3.5]nonan-2-yl)acetamido)-2-methylpyridin-3-yl)-2-bromopyrazolo[5,1-b]thiazole-7-carboxamide according to the procedure of Example 372, substituting 2-methoxypyridine-3-boronic acid for 3-fluoro-5-(4,4,5,5-tetramethyl-1,3,2-dioxaborolan-2-yl)pyridin-2-amine (17 mg, 32%). LCMS (ESI): mass calcd. for C₂₇H₂₉N₇O₄S, 547.6; m/z found, 548.2 [M+H]⁺. ¹H NMR (DMSO-d6) δ: 9.89 (s, 1H), 9.85 (br s, 1H), 8.99 (s, 1H), 8.58 (s, 1H), 8.56 (d, J=2.0 Hz, 1H), 8.22-8.29 (m, 2H), 8.17 (d, J=2.0 Hz, 1H), 7.17 (dd, J=7.3, 4.9 Hz, 1H), 4.07 (s, 3H), 3.46-3.52 (m, 4H), 3.29 (br s, 2H), 3.16 (br s, 4H), 2.41 (s, 3H), 1.66-1.74 (m, 4H).

### Example 404. N-(5-(2-(2-oxa-6-azaspiro[3.3]heptan-6-yl)acetamido)-2-methylpyridin-3-yl)-2-(2-methoxypyridin-3-yl)pyrazolo[5,1-b]thiazole-7-carboxamide

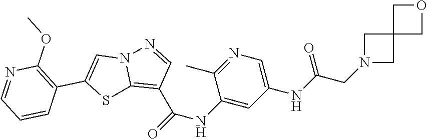 [SMILES: COc1ncccc1-c1cn2ncc(C(=O)Nc3cc(NC(=O)CN4CC5(COC5)C4)cnc3C)c2s1]

### Step a: N-(5-(2-(2-oxa-6-azaspiro[3.3]heptan-6-yl)acetamido)-2-methylpyridin-3-yl)-2-bromopyrazolo[5,1-b]thiazole-7-carboxamide

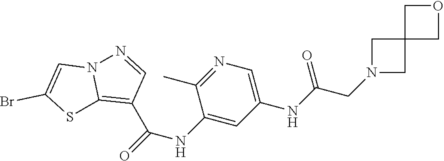 [SMILES: Cc1ncc(NC(=O)CN2CC3(COC3)C2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with 2-oxa-6-azaspiro[3.3]heptane (97 mg, 56%). LCMS (ESI): mass calcd. for C₁₉H₁₉BrN₆O₃S, 491.4; m/z found, 491.0/493.0 [M+H]⁺.

### Step b: N-(5-(2-(2-oxa-6-azaspiro[3.3]heptan-6-yl)acetamido)-2-methylpyridin-3-yl)-2-(2-methoxypyridin-3-yl)pyrazolo[5,1-b]thiazole-7-carboxamide

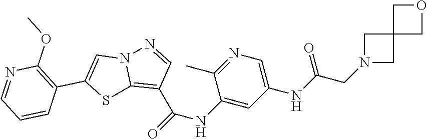 [SMILES: COc1ncccc1-c1cn2ncc(C(=O)Nc3cc(NC(=O)CN4CC5(COC5)C4)cnc3C)c2s1]

The title compound was prepared from N-(5-(2-(2-oxa-6-azaspiro[3.3]heptan-6-yl)acetamido)-2-methylpyridin-3-yl)-2-bromopyrazolo[5,1-b]thiazole-7-carboxamide according to the procedure of Example 372, substituting 2-methoxypyridine-3-boronic acid for 3-fluoro-5-(4,4,5,5-tetramethyl-1,3,2-dioxaborolan-2-yl)pyridin-2-amine (14 mg, 26%). LCMS (ESI): mass calcd. for C₂₅H₂₅N₇O₄S, 519.6; m/z found, 520.0 [M+H]⁺. ¹H NMR (DMSO-d6) δ: 9.89 (d, J=2.4 Hz, 2H), 8.99 (s, 1H), 8.57 (s, 1H), 8.55 (d, J=2.4 Hz, 1H), 8.23-8.27 (m, 2H), 8.17 (d, J=2.0 Hz, 1H), 7.17 (dd, J=7.6, 5.1 Hz, 1H), 4.63 (s, 4H), 4.07 (s, 3H), 3.47 (s, 4H), 3.31 (s, 7H), 3.20 (s, 2H), 2.41 (s, 3H).

### Example 405. 2-(2-methoxypyridin-3-yl)-N-(2-methyl-5-(2-(3-methylazetidin-1-yl)acetamido)pyridin-3-yl)pyrazolo[5,1-b]thiazole-7-carboxamide

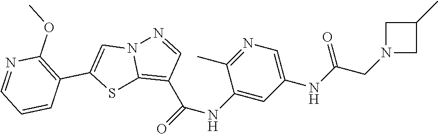 [SMILES: COc1ncccc1-c1cn2ncc(C(=O)Nc3cc(NC(=O)CN4CC(C)C4)cnc3C)c2s1]

### Step a: 2-bromo-N-(2-methyl-5-(2-(3-methylazetidin-1-yl)acetamido)pyridin-3-yl)pyrazolo[5,1-b]thiazole-7-carboxamide

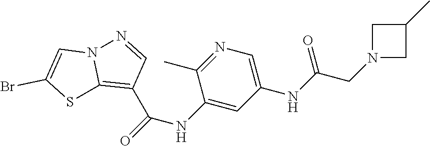 [SMILES: Cc1ncc(NC(=O)CN2CC(C)C2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with 3-methylazetidine hydrochloride (113 mg, 69%). LCMS (ESI): mass calcd. for C₁₈H₁₉BrN₆O₂S, 463.4; m/z found, 463.0/465.0 [M+H]⁺.

### Step b: 2-(2-methoxypyridin-3-yl)-N-(2-methyl-5-(2-(3-methylazetidin-1-yl)acetamido)pyridin-3-yl)pyrazolo[5,1-b]thiazole-7-carboxamide

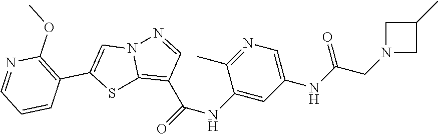 [SMILES: COc1ncccc1-c1cn2ncc(C(=O)Nc3cc(NC(=O)CN4CC(C)C4)cnc3C)c2s1]

The title compound was prepared from 2-bromo-N-(2-methyl-5-(2-(3-methylazetidin-1-yl)acetamido)pyridin-3-yl)pyrazolo[5,1-b]thiazole-7-carboxamide according to the procedure of Example 372, substituting 2-methoxypyridine-3-boronic acid for 3-fluoro-5-(4,4,5,5-tetramethyl-1,3,2-dioxaborolan-2-yl)pyridin-2-amine (16 mg, 30%). LCMS (ESI): mass calcd. for C₂₄H₂₅N₇O₃S, 491.6; m/z found, 492.1 [M+H]⁺. ¹H NMR (METHANOL-d4) δ: 8.66 (s, 1H), 8.56 (d, J=2.4 Hz, 1H), 8.44 (s, 1H), 8.24 (d, J=2.4 Hz, 1H), 8.20 (dd, J=4.9, 2.0 Hz, 1H), 8.08 (dd, J=7.6, 1.7 Hz, 1H), 7.11 (dd, J=7.3, 4.9 Hz, 1H), 4.11-4.20 (m, 5H), 4.00 (s, 2H), 3.66 (br t, J=8.8 Hz, 2H), 2.87-3.02 (m, 1H), 2.52 (s, 3H), 1.27 (d, J=6.8 Hz, 3H).

### Example 406. N-(5-(2-(7-oxa-2-azaspiro[3.5]nonan-2-yl)acetamido)-2-methylpyridin-3-yl)-2-(6,7-dihydro-5H-pyrazolo[5,1-b][1,3]oxazin-3-yl)pyrazolo[5,1-b]thiazole-7-carboxamide

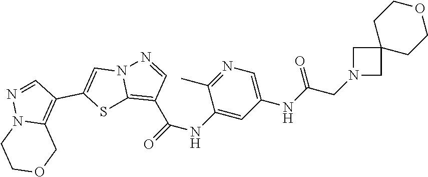 [SMILES: Cc1ncc(NC(=O)CN2CC3(CCOCC3)C2)cc1NC(=O)c1cnn2cc(-c3cnn4c3COCC4)sc12]

The title compound was prepared from N-(5-(2-(7-oxa-2-azaspiro[3.5]nonan-2-yl)acetamido)-2-methylpyridin-3-yl)-2-bromopyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (12 mg, 22%). LCMS (ESI): mass calcd. for C₂₇H₃₀N₈O₄S, 562.7; m/z found, 563.3 [M+H]⁺. ¹H NMR (METHANOL-d4) δ: 8.58 (d, J=2.4 Hz, 1H), 8.35 (s, 1H), 8.21 (d, J=2.4 Hz, 1H), 8.06 (s, 1H), 7.68 (s, 1H), 4.49-4.55 (m, 2H), 4.19 (t, J=6.1 Hz, 2H), 3.58-3.66 (m, 4H), 3.39 (s, 2H), 3.29 (s, 4H), 2.49 (s, 3H), 2.30-2.39 (m, 2H), 1.78-1.85 (m, 4H).

### Example 407. 2-(6,7-dihydro-5H-pyrazolo[5,1-b][1,3]oxazin-3-yl)-N-(2-methyl-5-(2-(3-methylazetidin-1-yl)acetamido)pyridin-3-yl)pyrazolo[5,1-b]thiazole-7-carboxamide

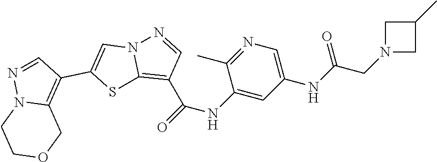 [SMILES: Cc1ncc(NC(=O)CN2CC(C)C2)cc1NC(=O)c1cnn2cc(-c3cnn4c3COCC4)sc12]

The title compound was prepared from 2-bromo-N-(2-methyl-5-(2-(3-methylazetidin-1-yl)acetamido)pyridin-3-yl)pyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (17 mg, 40%). LCMS (ESI): mass calcd. for C₂₄H₂₆N₈O₃S, 506.6; m/z found, 507.3 [M+H]⁺. ¹H NMR (METHANOL-d4) δ: 8.71 (d, J=2.4 Hz, 1H), 8.40 (d, J=2.0 Hz, 1H), 8.38 (s, 1H), 8.09 (s, 1H), 7.68 (s, 1H), 4.49-4.57 (m, 2H), 4.25-4.48 (m, 4H), 4.20 (t, J=6.1 Hz, 2H), 3.80-4.11 (m, 2H), 3.00-3.12 (m, 1H), 2.59 (s, 3H), 2.30-2.41 (m, 2H), 1.23-1.41 (m, 3H).

### Example 408. N-(5-(2-(1-azaspiro[3.3]heptan-1-yl)acetamido)-2-methylpyridin-3-yl)-2-(6,7-dihydro-5H-pyrazolo[5,1-b][1,3]oxazin-3-yl)pyrazolo[5,1-b]thiazole-7-carboxamide

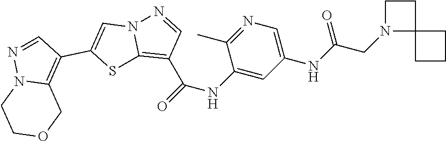 [SMILES: Cc1ncc(NC(=O)CN2CCC23CCC3)cc1NC(=O)c1cnn2cc(-c3cnn4c3COCC4)sc12]

### Step a: N-(5-(2-(1-azaspiro[3.3]heptan-1-yl)acetamido)-2-methylpyridin-3-yl)-2-bromopyrazolo[5,1-b]thiazole-7-carboxamide

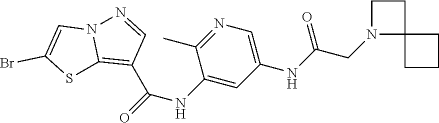 [SMILES: Cc1ncc(NC(=O)CN2CCC23CCC3)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with 1-azaspiro[3.3]heptane (49 mg, 27%). LCMS (ESI): mass calcd. for C₂₀H₂₁BrN₆O₂S, 489.4; m/z found, 489.1/491.1 [M+H]⁺.

### Step b: N-(5-(2-(1-azaspiro[3.3]heptan-1-yl)acetamido)-2-methylpyridin-3-yl)-2-(6,7-dihydro-5H-pyrazolo[5,1-b][1,3]oxazin-3-yl)pyrazolo[5,1-b]thiazole-7-carboxamide

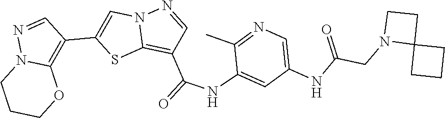 [SMILES: Cc1ncc(NC(=O)CN2CCC23CCC3)cc1NC(=O)c1cnn2cc(-c3cnn4c3OCCC4)sc12]

The title compound was prepared from 2-bromo-N-(2-methyl-5-(2-(3-methylazetidin-1-yl)acetamido)pyridin-3-yl)pyrazolo[5,1-b]thiazole-7-carboxamide according to the procedure of Example 372, substituting 2-methoxypyridine-3-boronic acid for 3-fluoro-5-(4,4,5,5-tetramethyl-1,3,2-dioxaborolan-2-yl)pyridin-2-amine (24 mg, 37%). LCMS (ESI): mass calcd. for C₂₆H₂₈N₈O₃S, 532.6; m/z found, 533.2 [M+H]⁺. ¹H NMR (METHANOL-d4) δ: 8.74 (d, J=2.0 Hz, 1H), 8.44 (d, J=2.4 Hz, 1H), 8.38 (s, 1H), 8.09 (s, 1H), 7.68 (s, 1H), 4.48-4.58 (m, 2H), 3.88-4.39 (m, 6H), 2.64-2.81 (m, 4H), 2.60 (s, 3H), 2.27-2.41 (m, 4H), 1.82-2.02 (m, 2H).

### Example 409. 2-(5,6-dihydro-4H-pyrrolo[1,2-b]pyrazol-3-yl)-N-(5-(2-(3,3-dimethylazetidin-1-yl)acetamido)-2-methylpyridin-3-yl)pyrazolo[5,1-b]thiazole-7-carboxamide

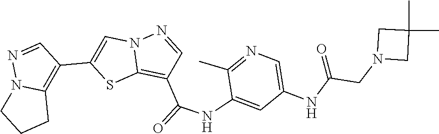 [SMILES: Cc1ncc(NC(=O)CN2CC(C)(C)C2)cc1NC(=O)c1cnn2cc(-c3cnn4c3CCC4)sc12]

The title compound was prepared from 2-bromo-N-(5-((2-(3,3-dimethylazetidin-1-yl)ethyl)carbamoyl)-2-methylpyridin-3-yl)pyrazolo[5,1-b]thiazole-7-carboxamide according to the procedure of Example 372, substituting 3-(tetramethyl-1,3,2-dioxaborolan-2-yl)-4H,5H,6H-pyrrolo[1,2-b]pyrazole for 3-fluoro-5-(4,4,5,5-tetramethyl-1,3,2-dioxaborolan-2-yl)pyridin-2-amine (28 mg, 43%). LCMS (ESI): mass calcd. for C₂₅H₂₈N₈O₂S, 504.6; m/z found, 505.1 [M+H]⁺. ¹H NMR (METHANOL-d4) δ: 8.67 (dd, J=3.9, 2.4 Hz, 1H), 8.40 (s, 1H), 8.37 (dd, J=4.4, 2.4 Hz, 1H), 8.17 (s, 1H), 7.83 (s, 1H), 4.31 (s, 2H), 4.19 (t, J=7.1 Hz, 2H), 3.93-4.16 (m, 4H), 3.08-3.15 (m, 2H), 2.70-2.80 (m, 2H), 2.57 (d, J=1.5 Hz, 3H), 1.31-1.51 (m, 6H).

### Example 410. N-(5-(2-(3,3-dimethylazetidin-1-yl)acetamido)-2-methylpyridin-3-yl)-2-(3-methylpyridazin-4-yl)pyrazolo[5,1-b]thiazole-7-carboxamide

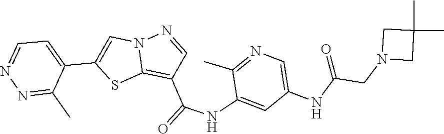 [SMILES: Cc1ncc(NC(=O)CN2CC(C)(C)C2)cc1NC(=O)c1cnn2cc(-c3ccnnc3C)sc12]

The title compound was prepared from 2-bromo-N-(5-((2-(3,3-dimethylazetidin-1-yl)ethyl)carbamoyl)-2-methylpyridin-3-yl)pyrazolo[5,1-b]thiazole-7-carboxamide according to the procedure of Example 372, substituting 3-methyl-5-(4,4,5,5-tetramethyl-1,3,2-dioxaborolan-2-yl)pyridazine for 3-fluoro-5-(4,4,5,5-tetramethyl-1,3,2-dioxaborolan-2-yl)pyridin-2-amine (7 mg, 28%). LCMS (ESI): mass calcd. for C₂₄H₂₆N₈O₂S, 490.6; m/z found, 491.2 [M+H]⁺. ¹H NMR (METHANOL-d4) δ: 9.46 (d, J=2.0 Hz, 1H), 9.09 (s, 1H), 8.65 (d, J=2.4 Hz, 1H), 8.54 (s, 1H), 8.35 (d, J=2.4 Hz, 1H), 7.94 (d, J=2.0 Hz, 1H), 4.31 (s, 2H), 3.92-4.18 (m, 4H), 2.77 (s, 3H), 2.56 (s, 3H), 1.32-1.51 (m, 6H).

### Example 411. N-(5-(2-(2-oxa-6-azaspiro[3.3]heptan-6-yl)acetamido)-2-methylpyridin-3-yl)-2-(6,7-dihydro-5H-pyrazolo[5,1-b][1,3]oxazin-3-yl)pyrazolo[5,1-b]thiazole-7-carboxamide

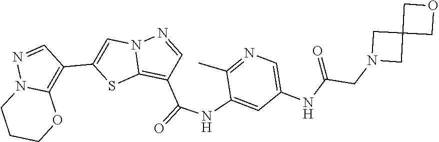 [SMILES: Cc1ncc(NC(=O)CN2CC3(COC3)C2)cc1NC(=O)c1cnn2cc(-c3cnn4c3OCCC4)sc12]

The title compound was prepared from N-(5-(2-(2-oxa-6-azaspiro[3.3]heptan-6-yl)acetamido)-2-methylpyridin-3-yl)-2-bromopyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (5 mg, 9%). LCMS (ESI): mass calcd. for C₂₅H₂₆N₈O₄S, 534.6; m/z found, 535.2 [M+H]⁺. ¹H NMR (METHANOL-d4) δ: 8.57 (d, J=2.4 Hz, 1H), 8.35 (s, 1H), 8.20 (d, J=2.4 Hz, 1H), 8.06 (s, 1H), 7.68 (s, 1H), 4.77 (s, 4H), 4.48-4.55 (m, 2H), 4.19 (t, J=6.1 Hz, 2H), 3.59 (s, 4H), 2.49 (s, 3H), 2.30-2.39 (m, 2H).

### Example 412. N-(5-(2-(azetidin-1-yl)acetamido)-2-methylpyridin-3-yl)-2-(6,7-dihydro-5H-pyrazolo[5,1-b][1,3]oxazin-3-yl)pyrazolo[5,1-b]thiazole-7-carboxamide

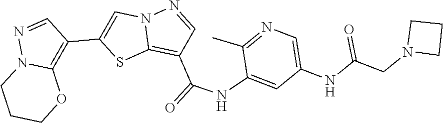 [SMILES: Cc1ncc(NC(=O)CN2CCC2)cc1NC(=O)c1cnn2cc(-c3cnn4c3OCCC4)sc12]

The title compound was prepared from N-(5-(2-(azetidin-1-yl)acetamido)-2-methylpyridin-3-yl)-2-bromopyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (12 mg, 17%). LCMS (ESI): mass calcd. for C₂₃H₂₄N₈O₃S, 492.6; m/z found, 493.1 [M+H]⁺. ¹H NMR (METHANOL-d4) δ: 8.68 (d, J=2.4 Hz, 1H), 8.35-8.40 (m, 2H), 8.08 (s, 1H), 7.68 (s, 1H), 4.50-4.56 (m, 2H), 4.16-4.46 (m, 8H), 2.44-2.74 (m, 5H), 2.31-2.40 (m, 2H).

### Example 413. (S)-2-(6,7-dihydro-5H-pyrazolo[5,1-b][1,3]oxazin-3-yl)-N-(2-methyl-5-(2-(2-methylpyrrolidin-1-yl)acetamido)pyridin-3-yl)pyrazolo[5,1-b]thiazole-7-carboxamide

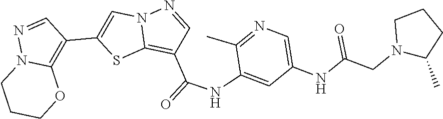 [SMILES: Cc1ncc(NC(=O)CN2CCC[C@@H]2C)cc1NC(=O)c1cnn2cc(-c3cnn4c3OCCC4)sc12]

### Step a: (S)-2-bromo-N-(2-methyl-5-(2-(2-methylpyrrolidin-1-yl)acetamido)pyridin-3-yl)pyrazolo[5,1-b]thiazole-7-carboxamide

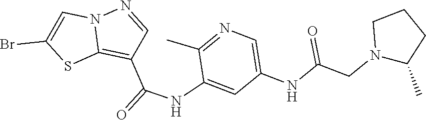 [SMILES: Cc1ncc(NC(=O)CN2CCC[C@@H]2C)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with (S)-2-methyl-pyrrolidine (124 mg, 74%). LCMS (ESI): mass calcd. for C₁₉H₂₁BrN₆O₂S, 477.4; m/z found, 477.0/479.0 [M+H]⁺.

### Step b: (S)-2-(6,7-dihydro-5H-pyrazolo[5,1-b][1,3]oxazin-3-yl)-N-(2-methyl-5-(2-(2-methylpyrrolidin-1-yl)acetamido)pyridin-3-yl)pyrazolo[5,1-b]thiazole-7-carboxamide

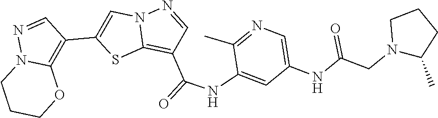 [SMILES: Cc1ncc(NC(=O)CN2CCC[C@@H]2C)cc1NC(=O)c1cnn2cc(-c3cnn4c3OCCC4)sc12]

The title compound was prepared from (S)-2-bromo-N-(2-methyl-5-(2-(2-methylpyrrolidin-1-yl)acetamido)pyridin-3-yl)pyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (27 mg, 40%). LCMS (ESI): mass calcd. for C₂₅H₂₈N₈O₃S, 520.6; m/z found, 521.2 [M+H]⁺. ¹H NMR (METHANOL-d4) δ: 8.69 (d, J=2.4 Hz, 1H), 8.36-8.40 (m, 2H), 8.09 (s, 1H), 7.68 (s, 1H), 4.49-4.56 (m, 2H), 4.41 (br d, J=16.1 Hz, 1H), 4.20 (t, J=6.1 Hz, 2H), 4.09 (br d, J=16.1 Hz, 1H), 3.92-4.02 (m, 1H), 3.55-3.67 (m, 1H), 2.57 (s, 3H), 2.31-2.40 (m, 3H), 2.07-2.22 (m, 2H), 1.73-1.88 (m, 1H), 1.49 (br d, J=6.4 Hz, 3H).

### Example 414. 2-(6,7-dihydro-5H-pyrazolo[5,1-b][1,3]oxazin-3-yl)-N-(5-(2-(isobutylamino)acetamido)-2-methylpyridin-3-yl)pyrazolo[5,1-b]thiazole-7-carboxamide

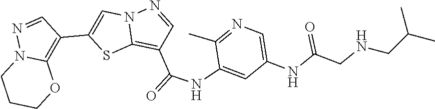 [SMILES: Cc1ncc(NC(=O)CNCC(C)C)cc1NC(=O)c1cnn2cc(-c3cnn4c3OCCC4)sc12]

### Step a: 2-bromo-N-(5-(2-(isobutylamino)acetamido)-2-methylpyridin-3-yl)pyrazolo[5,1-b]thiazole-7-carboxamide

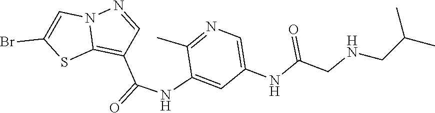 [SMILES: Cc1ncc(NC(=O)CNCC(C)C)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethyl azetidine hydrochloride with isobutylamine (85 mg, 52%). LCMS (ESI): mass calcd. for C₁₈H₂₁BrN₆O₂S, 465.4; m/z found, 465.0/467.0 [M+H]⁺.

### Step b: 2-(6,7-dihydro-5H-pyrazolo[5,1-b][1,3]oxazin-3-yl)-N-(5-(2-(isobutylamino)acetamido)-2-methylpyridin-3-yl)pyrazolo[5,1-b]thiazole-7-carboxamide

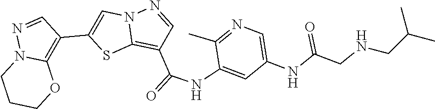 [SMILES: Cc1ncc(NC(=O)CNCC(C)C)cc1NC(=O)c1cnn2cc(-c3cnn4c3OCCC4)sc12]

The title compound was prepared from 2-bromo-N-(5-(2-(isobutylamino)acetamido)-2-methylpyridin-3-yl)pyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (85 mg, 52%). LCMS (ESI): mass calcd. for C₂₄H₂₈N₈O₃S, 508.6; m/z found, 509.2 [M+H]⁺. ¹H NMR (METHANOL-d4) δ: 8.69 (d, J=2.0 Hz, 1H), 8.34-8.40 (m, 2H), 8.07-8.11 (m, 1H), 7.68 (s, 1H), 4.50-4.55 (m, 2H), 4.20 (t, J=6.1 Hz, 2H), 4.04 (s, 2H), 2.97 (d, J=7.3 Hz, 2H), 2.57 (s, 3H), 2.30-2.39 (m, 2H), 2.01-2.15 (m, 1H), 1.08 (d, J=6.4 Hz, 6H).

### Example 415. 2-(5,6-dihydro-8H-imidazo[2,1-c][1,4]oxazin-3-yl)-N-(5-(2-(3,3-dimethylazetidin-1-yl)acetamido)-2-methylpyridin-3-yl)pyrazolo[5,1-b]thiazole-7-carboxamide

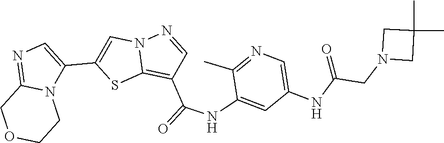 [SMILES: Cc1ncc(NC(=O)CN2CC(C)(C)C2)cc1NC(=O)c1cnn2cc(-c3cnc4n3CCOC4)sc12]

The title compound was prepared from 2-bromo-N-(5-((2-(3,3-dimethylazetidin-1-yl)ethyl)carbamoyl)-2-methylpyridin-3-yl)pyrazolo[5,1-b]thiazole-7-carboxamide according to the procedure of Example 372, substituting 5,6-dihydro-8H-imidazo[2,1-c][1,4]oxazine-3-boronic acid pinacol ester for 3-fluoro-5-(4,4,5,5-tetramethyl-1,3,2-dioxaborolan-2-yl)pyridin-2-amine (9 mg, 14%). LCMS (ESI): mass calcd. for C₂₅H₂₈N₈O₃S, 520.6; m/z found, 521.2 [M+H]⁺. ¹H NMR (METHANOL-d4) δ: 8.65 (d, J=2.0 Hz, 1H), 8.52 (s, 2H), 8.33 (d, J=2.4 Hz, 1H), 7.78 (s, 1H), 5.04 (s, 2H), 4.25-4.34 (m, 4H), 4.18-4.24 (m, 2H), 3.92-4.16 (m, 4H), 2.55 (s, 3H), 1.29-1.52 (m, 6H).

### Example 416. (S)-2-(5,6-dihydro-4H-pyrrolo[1,2-b]pyrazol-3-yl)-N-(2-methyl-5-(2-(2-methylpyrrolidin-1-yl)acetamido)pyridin-3-yl)pyrazolo[5,1-b]thiazole-7-carboxamide

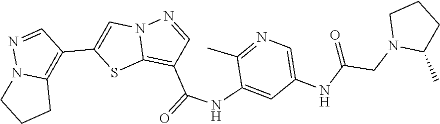 [SMILES: Cc1ncc(NC(=O)CN2CCC[C@@H]2C)cc1NC(=O)c1cnn2cc(-c3cnn4c3CCC4)sc12]

The title compound was prepared from (S)-2-bromo-N-(2-methyl-5-(2-(2-methylpyrrolidin-1-yl)acetamido)pyridin-3-yl)pyrazolo[5,1-b]thiazole-7-carboxamide according to the procedure of Example 372, substituting 3-(tetramethyl-1,3,2-dioxaborolan-2-yl)-4H,5H,6H-pyrrolo[1,2-b]pyrazole for 3-fluoro-5-(4,4,5,5-tetramethyl-1,3,2-dioxaborolan-2-yl)pyridin-2-amine (14 mg, 43%). LCMS (ESI): mass calcd. for C₂₅H₂₈N₈O₂S, 504.6; m/z found, 505.2 [M+H]⁺. ¹H NMR (METHANOL-d4) δ: 8.74 (d, J=2.4 Hz, 1H), 8.43-8.46 (m, 1H), 8.40 (s, 1H), 8.17 (s, 1H), 7.83 (s, 1H), 4.42 (br d, J=16.1 Hz, 1H), 4.19 (t, J=7.1 Hz, 2H), 4.07-4.15 (m, 1H), 3.97 (dt, J=12.3, 6.3 Hz, 1H), 3.54-3.68 (m, 1H), 3.21-3.27 (m, 1H), 3.08-3.15 (m, 2H), 2.70-2.80 (m, 2H), 2.60 (s, 3H), 2.36 (dq, J=12.9, 6.5 Hz, 1H), 2.07-2.22 (m, 2H), 1.73-1.89 (m, 1H), 1.49 (br d, J=6.4 Hz, 3H).

### Example 417. N-(5-(2-(5-azaspiro[3.4]octan-5-yl)acetamido)-2-methylpyridin-3-yl)-2-(6,7-dihydro-4H-pyrazolo[5,1-c][1,4]oxazin-3-yl)pyrazolo[5,1-b]thiazole-7-carboxamide

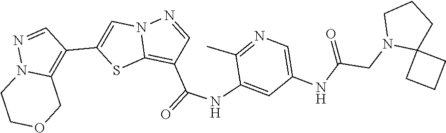 [SMILES: Cc1ncc(NC(=O)CN2CCCC23CCC3)cc1NC(=O)c1cnn2cc(-c3cnn4c3COCC4)sc12]

### Step a: N-(5-(2-(5-azaspiro[3.4]octan-5-yl)acetamido)-2-methylpyridin-3-yl)-2-bromopyrazolo[5,1-b]thiazole-7-carboxamide

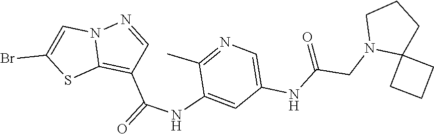 [SMILES: Cc1ncc(NC(=O)CN2CCCC23CCC3)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with 5-azaspiro[3.4]octane (144 mg, 73%). LCMS (ESI): mass calcd. for C₂₁H₂₃BrN₆O₂S, 503.4; m/z found, 503.1/505.1 [M+H].

### Step b: 3-(4,4,5,5-tetramethyl-1,3,2-dioxaborolan-2-yl)-6,7-dihydro-4H-pyrazolo[5,1-c][1,4]oxazine

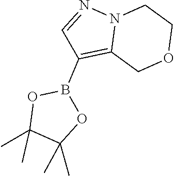 [SMILES: CC1(C)OB(c2cnn3c2COCC3)OC1(C)C]

A mixture of 3-bromo-6,7-dihydro-4H-pyrazolo[5,1-c][1,4]oxazine (106 mg, 0.52 mmol), bis(pinacolato)diboron (146 mg, 0.57 mmol), KOAc (173 mg, 1.76 mmol), and Xphos Pd G4 (22 mg, 0.026 mmol) in 1,4-dioxane (3.5 mL) under nitrogen in a capped 5 mL microwave vial was sparged with nitrogen for 10 min then heated to 95° C. in a heating block overnight. The reaction was cooled to rt, filtered, and concentrated. The residue was taken up in DCM and loaded onto a pre-column and purified by flash column, 0-75% EtOAc/heptane to yield 3-(4,4,5,5-tetramethyl-1,3,2-dioxaborolan-2-yl)-6,7-dihydro-4H-pyrazolo[5,1-c][1,4]oxazine (31 mg, 24%) as a tacky white solid. LCMS (ESI): mass calcd. for C₁₂H₁₉BN₂O₃, 250.1; m/z found, 251.2 [M+H]⁺.

### Step c: N-(5-(2-(5-azaspiro[3.4]octan-5-yl)acetamido)-2-methylpyridin-3-yl)-2-(6,7-dihydro-4H-pyrazolo[5,1-c][1,4]oxazin-3-yl)pyrazolo[5,1-b]thiazole-7-carboxamide

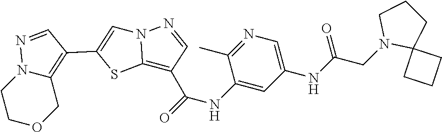 [SMILES: Cc1ncc(NC(=O)CN2CCCC23CCC3)cc1NC(=O)c1cnn2cc(-c3cnn4c3COCC4)sc12]

The title compound was prepared from N-(5-(2-(5-azaspiro[3.4]octan-5-yl)acetamido)-2-methylpyridin-3-yl)-2-bromopyrazolo[5,1-b]thiazole-7-carboxamide according to the procedure of Example 372, substituting 3-(4,4,5,5-tetramethyl-1,3,2-dioxaborolan-2-yl)-6,7-dihydro-4H-pyrazolo[5,1-c][1,4]oxazine for 3-fluoro-5-(4,4,5,5-tetramethyl-1,3,2-dioxaborolan-2-yl)pyridin-2-amine (20 mg, 30%). LCMS (ESI): mass calcd. for C₂₇H₃₀N₈O₃S, 546.7; m/z found, 547.2 [M+H]⁺. ¹H NMR (METHANOL-d4) δ: 8.71 (d, J=2.4 Hz, 1H), 8.40-8.45 (m, 2H), 8.10 (s, 1H), 7.82 (s, 1H), 5.05 (s, 2H), 3.72-4.46 (m, 8H), 2.54-2.68 (m, 5H), 2.33 (br s, 2H), 2.04-2.21 (m, 4H), 1.90-2.02 (m, 2H).

### Example 418. N-(5-(2-(5-azaspiro[3.4]octan-5-yl)acetamido)-2-methylpyridin-3-yl)-2-(5,6-dihydro-8H-imidazo[2,1-c][1,4]oxazin-3-yl)pyrazolo[5,1-b]thiazole-7-carboxamide

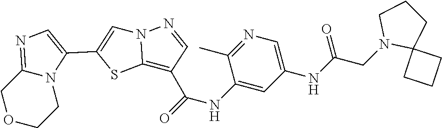 [SMILES: Cc1ncc(NC(=O)CN2CCCC23CCC3)cc1NC(=O)c1cnn2cc(-c3cnc4n3CCOC4)sc12]

The title compound was prepared from N-(5-(2-(5-azaspiro[3.4]octan-5-yl)acetamido)-2-methylpyridin-3-yl)-2-bromopyrazolo[5,1-b]thiazole-7-carboxamide according to the procedure of Example 372, substituting 5,6-dihydro-8H-imidazo[2,1-c][1,4]oxazine-3-boronic acid pinacol ester for 3-fluoro-5-(4,4,5,5-tetramethyl-1,3,2-dioxaborolan-2-yl)pyridin-2-amine (5 mg, 8%). LCMS (ESI): mass calcd. for C₂₇H₃₀N₈O₃S, 546.7; m/z found, 547.3 [M+H]⁺. ¹H NMR (METHANOL-d4) δ: 8.65 (d, J=2.4 Hz, 1H), 8.51 (d, J=3.9 Hz, 2H), 8.34 (d, J=2.4 Hz, 1H), 7.75 (s, 1H), 5.02 (s, 2H), 3.74-4.44 (m, 8H), 2.52-2.66 (m, 5H), 2.33 (br s, 2H), 2.05-2.19 (m, 4H), 1.91-2.01 (m, 2H).

### Example 419. 2-(6,7-dihydro-5H-pyrazolo[5,1-b][1,3]oxazin-3-yl)-N-(2-methyl-5-(2-(piperidin-1-yl)acetamido)pyridin-3-yl)pyrazolo[5,1-b]thiazole-7-carboxamide

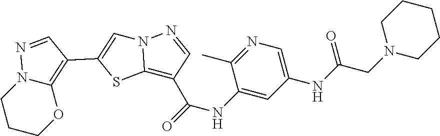 [SMILES: Cc1ncc(NC(=O)CN2CCCCC2)cc1NC(=O)c1cnn2cc(-c3cnn4c3OCCC4)sc12]

### Step a: 2-bromo-N-(2-methyl-5-(2-(piperidin-1-yl)acetamido)pyridin-3-yl)pyrazolo[5,1-b]thiazole-7-carboxamide

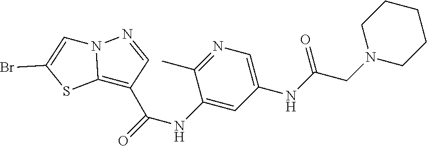 [SMILES: Cc1ncc(NC(=O)CN2CCCCC2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with piperidine (124 mg, 66%). LCMS (ESI): mass calcd. for C₁₉H₂₁BrN₆O₂S, 477.4; m/z found, 477.0/479.0 [M+H]⁺.

### Step b: 2-(6,7-dihydro-5H-pyrazolo[5,1-b][1,3]oxazin-3-yl)-N-(2-methyl-5-(2-(piperidin-1-yl)acetamido)pyridin-3-yl)pyrazolo[5,1-b]thiazole-7-carboxamide

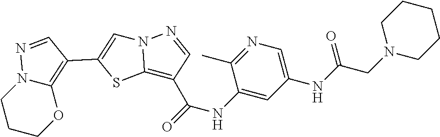 [SMILES: Cc1ncc(NC(=O)CN2CCCCC2)cc1NC(=O)c1cnn2cc(-c3cnn4c3OCCC4)sc12]

The title compound was prepared from 2-bromo-N-(2-methyl-5-(2-(piperidin-1-yl)acetamido)pyridin-3-yl)pyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (23 mg, 35%). LCMS (ESI): mass calcd. for C₂₅H₂₈N₈O₃S, 520.6; m/z found, 521.2 [M+H]⁺. ¹H NMR (METHANOL-d4) δ: 8.71 (d, J=2.0 Hz, 1H), 8.41 (d, J=2.0 Hz, 1H), 8.38 (s, 1H), 8.09 (s, 1H), 7.68 (s, 1H), 4.52 (t, J=5.1 Hz, 2H), 4.20 (t, J=6.1 Hz, 2H), 4.14 (s, 2H), 3.64 (br dd, J=6.6, 4.2 Hz, 2H), 3.07-3.15 (m, 2H), 2.55-2.62 (m, 3H), 2.35 (quin, J=5.5 Hz, 2H), 1.46-2.06 (m, 6H).

### Example 420. N-(5-(2-(azepan-1-yl)acetamido)-2-methylpyridin-3-yl)-2-(6,7-dihydro-5H-pyrazolo[5,1-b][1,3]oxazin-3-yl)pyrazolo[5,1-b]thiazole-7-carboxamide

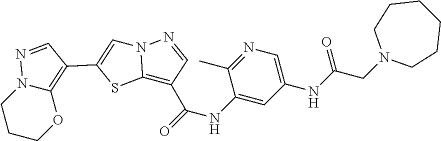 [SMILES: Cc1ncc(NC(=O)CN2CCCCCC2)cc1NC(=O)c1cnn2cc(-c3cnn4c3OCCC4)sc12]

### Step a: N-(5-(2-(azepan-1-yl)acetamido)-2-methylpyridin-3-yl)-2-bromopyrazolo[5,1-b]thiazole-7-carboxamide

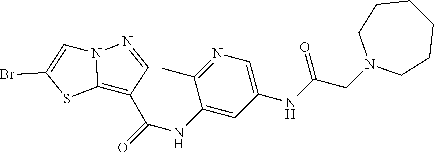 [SMILES: Cc1ncc(NC(=O)CN2CCCCCC2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with hexamethyleneimine (118 mg, 63%). LCMS (ESI): mass calcd. for C₂₀H₂₃BrN₆O₂S, 491.4; m/z found, 491.1/493.1 [M+H]⁺.

### Step b: N-(5-(2-(azepan-1-yl)acetamido)-2-methylpyridin-3-yl)-2-(6,7-dihydro-5H-pyrazolo[5,1-b][1,3]oxazin-3-yl)pyrazolo[5,1-b]thiazole-7-carboxamide

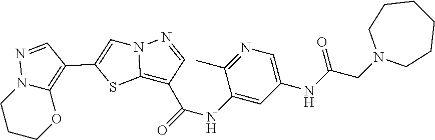 [SMILES: Cc1ncc(NC(=O)CN2CCCCCC2)cc1NC(=O)c1cnn2cc(-c3cnn4c3OCCC4)sc12]

The title compound was prepared from 2 N-(5-(2-(azepan-1-yl)acetamido)-2-methylpyridin-3-yl)-2-bromopyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (25 mg, 37%). LCMS (ESI): mass calcd. for C₂₆H₃₀N₈O₃S, 534.6; m/z found, 535.2 [M+H]⁺. ¹H NMR (METHANOL-d4) δ: 8.71 (d, J=2.4 Hz, 1H), 8.39-8.45 (m, 2H), 8.10 (s, 1H), 7.82 (s, 1H), 5.05 (s, 2H), 4.14-4.26 (m, 6H), 3.55 (br d, J=1.5 Hz, 2H), 3.36 (br s, 2H), 2.58 (s, 3H), 1.98 (br s, 4H), 1.78 (br s, 4H).

### Example 421. N-(5-(2-(cyclopentylamino)acetamido)-2-methylpyridin-3-yl)-2-(6,7-dihydro-5H-pyrazolo[5,1-b][1,3]oxazin-3-yl)pyrazolo[5,1-b]thiazole-7-carboxamide

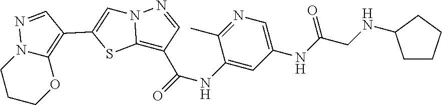 [SMILES: Cc1ncc(NC(=O)CNC2CCCC2)cc1NC(=O)c1cnn2cc(-c3cnn4c3OCCC4)sc12]

### Step a: 2-bromo-N-(5-(2-(cyclopentylamino)acetamido)-2-methylpyridin-3-yl)pyrazolo[5,1-b]thiazole-7-carboxamide

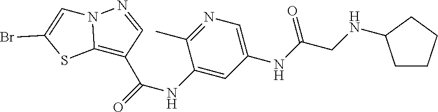 [SMILES: Cc1ncc(NC(=O)CNC2CCCC2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with cyclopentylamine (132 mg, 69%). LCMS (ESI): mass calcd. for C₁₉H₂₁BrN₆O₂S, 477.4; m/z found, 477.1/479.1 [M+H]⁺.

### Step b: N-(5-(2-(cyclopentylamino)acetamido)-2-methylpyridin-3-yl)-2-(6,7-dihydro-5H-pyrazolo[5,1-b][1,3]oxazin-3-yl)pyrazolo[5,1-b]thiazole-7-carboxamide

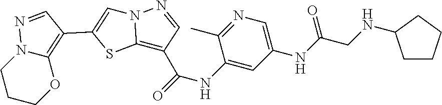 [SMILES: Cc1ncc(NC(=O)CNC2CCCC2)cc1NC(=O)c1cnn2cc(-c3cnn4c3OCCC4)sc12]

The title compound was prepared from N-(5-(2-(cyclopentylamino)acetamido)-2-methylpyridin-3-yl)-2-(6,7-dihydro-5H-pyrazolo[5,1-b][1,3]oxazin-3-yl)pyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (25 mg, 37%). LCMS (ESI): mass calcd. for C₂₅H₂₈N₈O₃S, 520.6; m/z found, 521.1 [M+H]⁺. ¹H NMR (METHANOL-d4) δ: 8.77 (d, J=2.0 Hz, 1H), 8.45 (d, J=2.0 Hz, 1H), 8.38 (s, 1H), 8.07-8.11 (m, 1H), 7.68 (s, 1H), 4.49-4.56 (m, 2H), 4.20 (t, J=6.1 Hz, 2H), 4.05 (s, 2H), 3.65 (quin, J=7.5 Hz, 1H), 2.58-2.63 (m, 3H), 2.30-2.39 (m, 2H), 2.10-2.23 (m, 2H), 1.79-1.91 (m, 2H), 1.64-1.77 (m, 4H).

### Example 422. N-(5-(2-(cyclopentylamino)acetamido)-2-methylpyridin-3-yl)-2-(5,6-dihydro-4H-pyrrolo[1,2-b]pyrazol-3-yl)pyrazolo[5,1-b]thiazole-7-carboxamide

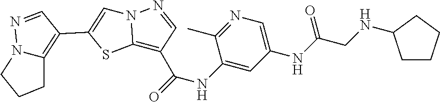 [SMILES: Cc1ncc(NC(=O)CNC2CCCC2)cc1NC(=O)c1cnn2cc(-c3cnn4c3CCC4)sc12]

The title compound was prepared from N-(5-(2-(cyclopentylamino)acetamido)-2-methylpyridin-3-yl)-2-(6,7-dihydro-5H-pyrazolo[5,1-b][1,3]oxazin-3-yl)pyrazolo[5,1-b]thiazole-7-carboxamide according to the procedure of Example 372, substituting 3-(tetramethyl-1,3,2-dioxaborolan-2-yl)-4H,5H,6H-pyrrolo[1,2-b]pyrazole for 3-fluoro-5-(4,4,5,5-tetramethyl-1,3,2-dioxaborolan-2-yl)pyridin-2-amine (23 mg, 36%). LCMS (ESI): mass calcd. for C₂₅H₂₈N₈O₂S, 504.6; m/z found, 505.1 [M+H]⁺. ¹H NMR (METHANOL-d4) δ: 8.71 (d, J=2.4 Hz, 1H), 8.39-8.43 (m, 2H), 8.17 (s, 1H), 7.83 (s, 1H), 4.16-4.23 (m, 2H), 4.04 (s, 2H), 3.60-3.70 (m, 1H), 3.08-3.15 (m, 2H), 2.75 (quin, J=7.3 Hz, 2H), 2.58 (s, 3H), 2.11-2.22 (m, 2H), 1.78-1.91 (m, 2H), 1.63-1.77 (m, 4H).

### Example 423. N-(5-(2-(5-oxa-2-azaspiro[3.5]nonan-2-yl)acetamido)-2-methylpyridin-3-yl)-2-(6,7-dihydro-5H-pyrazolo[5,1-b][1,3]oxazin-3-yl)pyrazolo[5,1-b]thiazole-7-carboxamide

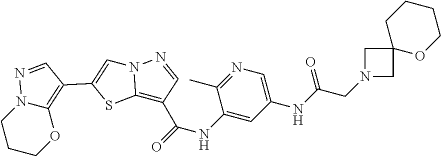 [SMILES: Cc1ncc(NC(=O)CN2CC3(CCCCO3)C2)cc1NC(=O)c1cnn2cc(-c3cnn4c3OCCC4)sc12]

### Step a: N-(5-(2-(5-oxa-2-azaspiro[3.5]nonan-2-yl)acetamido)-2-methylpyridin-3-yl)-2-bromopyrazolo[5,1-b]thiazole-7-carboxamide

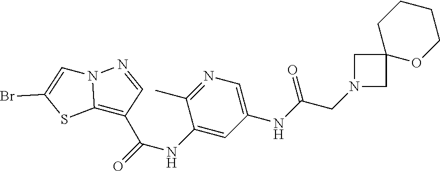 [SMILES: Cc1ncc(NC(=O)CN2CC3(CCCCO3)C2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with 5-oxa-2-azaspiro[3.5]nonane hydrochloride (105 mg, 56%). LCMS (ESI): mass calcd. for C₂₁H₂₃BrN₆O₃S, 519.4; m/z found, 519.1/521.1 [M+H]⁺.

### Step b: N-(5-(2-(5-oxa-2-azaspiro[3.5]nonan-2-yl)acetamido)-2-methylpyridin-3-yl)-2-(6,7-dihydro-5H-pyrazolo[5,1-b][1,3]oxazin-3-yl)pyrazolo[5,1-b]thiazole-7-carboxamide

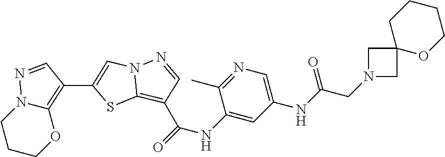 [SMILES: Cc1ncc(NC(=O)CN2CC3(CCCCO3)C2)cc1NC(=O)c1cnn2cc(-c3cnn4c3OCCC4)sc12]

The title compound was prepared from N-(5-(2-(5-oxa-2-azaspiro[3.5]nonan-2-yl)acetamido)-2-methylpyridin-3-yl)-2-bromopyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (30 mg, 44%). LCMS (ESI): mass calcd. for C₂₇H₃₀N₈O₄S, 562.7; m/z found, 563.2 [M+H]⁺. ¹H NMR (METHANOL-d4) δ: 8.78 (d, J=2.0 Hz, 1H), 8.49 (d, J=2.0 Hz, 1H), 8.38 (s, 1H), 8.09 (s, 1H), 7.68 (s, 1H), 4.50-4.55 (m, 2H), 4.11-4.44 (m, 8H), 3.73 (br s, 2H), 2.62 (s, 3H), 2.30-2.40 (m, 2H), 1.81-1.89 (m, 2H), 1.66 (br s, 2H), 1.52-1.61 (m, 2H).

### Example 424. N-(5-(2-(5-oxa-2-azaspiro[3.5]nonan-2-yl)acetamido)-2-methylpyridin-3-yl)-2-(5,6-dihydro-4H-pyrrolo[1,2-b]pyrazol-3-yl)pyrazolo[5,1-b]thiazole-7-carboxamide

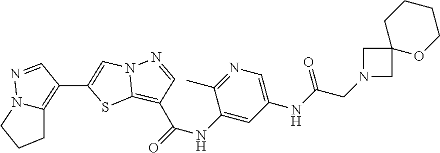 [SMILES: Cc1ncc(NC(=O)CN2CC3(CCCCO3)C2)cc1NC(=O)c1cnn2cc(-c3cnn4c3CCC4)sc12]

The title compound was prepared from N-(5-(2-(5-oxa-2-azaspiro[3.5]nonan-2-yl)acetamido)-2-methylpyridin-3-yl)-2-bromopyrazolo[5,1-b]thiazole-7-carboxamide according to the procedure of Example 372, substituting 3-(tetramethyl-1,3,2-dioxaborolan-2-yl)-4H,5H,6H-pyrrolo[1,2-b]pyrazole for 3-fluoro-5-(4,4,5,5-tetramethyl-1,3,2-dioxaborolan-2-yl)pyridin-2-amine (29 mg, 43%). LCMS (ESI): mass calcd. for C₂₇H₃₀N₈O₃S, 546.7; m/z found, 547.2 [M+H]⁺. ¹H NMR (METHANOL-d4) δ: 8.68 (d, J=2.0 Hz, 1H), 8.37-8.42 (m, 2H), 8.17 (s, 1H), 7.83 (s, 1H), 4.08-4.46 (m, 8H), 3.73 (br s, 2H), 3.08-3.16 (m, 2H), 2.70-2.81 (m, 2H), 2.58 (s, 3H), 1.81-1.89 (m, 2H), 1.67 (br s, 2H), 1.52-1.61 (m, 2H).

### Example 425. 2-(3,5-dimethyl-1-(oxetan-3-yl)-1H-pyrazol-4-yl)-N-(5-(2-(3,3-dimethylazetidin-1-yl)acetamido)-2-methylpyridin-3-yl)pyrazolo[5,1-b]thiazole-7-carboxamide

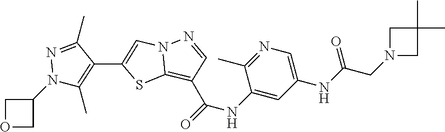 [SMILES: Cc1ncc(NC(=O)CN2CC(C)(C)C2)cc1NC(=O)c1cnn2cc(-c3c(C)nn(C4COC4)c3C)sc12]

The title compound was prepared from 2-bromo-N-(5-((2-(3,3-dimethylazetidin-1-yl)ethyl)carbamoyl)-2-methylpyridin-3-yl)pyrazolo[5,1-b]thiazole-7-carboxamide according to the procedure of Example 372, substituting 3,5-dimethyl-1-(oxetan-3-yl)-4-(4,4,5,5-tetramethyl-1,3,2-dioxaborolan-2-yl)-1H-pyrazole for 3-fluoro-5-(4,4,5,5-tetramethyl-1,3,2-dioxaborolan-2-yl)pyridin-2-amine (30 mg, 40%). LCMS (ESI): mass calcd. for C₂₇H₃₂N₈O₃S, 548.7; m/z found, 549.3 [M+H]⁺. ¹H NMR (METHANOL-d4) δ: 8.72-8.80 (m, 1H), 8.43-8.53 (m, 2H), 8.05 (s, 1H), 5.62 (quin, J=7.1 Hz, 1H), 5.11 (br t, J=6.1 Hz, 2H), 4.98-5.06 (m, 2H), 4.33 (br s, 2H), 3.91-4.19 (m, 4H), 2.62 (br d, J=4.9 Hz, 3H), 2.35 (br d, J=11.7 Hz, 6H), 1.31-1.53 (m, 6H).

### Example 426. N-(5-(2-(azetidin-3-yl)acetamido)-2-methylpyridin-3-yl)-2-(5,6-dihydro-4H-pyrrolo[1,2-b]pyrazol-3-yl)pyrazolo[5,1-b]thiazole-7-carboxamide

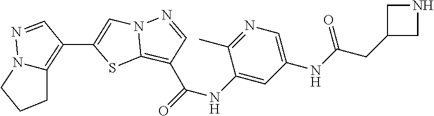 [SMILES: Cc1ncc(NC(=O)CC2CNC2)cc1NC(=O)c1cnn2cc(-c3cnn4c3CCC4)sc12]

### Step a: tert-butyl 3-(2-((5-(2-bromopyrazolo[5,1-b]thiazole-7-carboxamido)-6-methylpyridin-3-yl)amino)-2-oxoethyl)azetidine-1-carboxylate

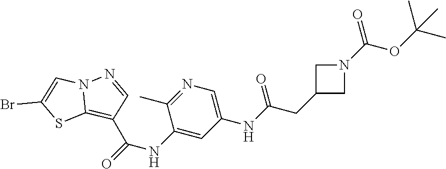 [SMILES: Cc1ncc(NC(=O)CC2CN(C(=O)OC(C)(C)C)C2)cc1NC(=O)c1cnn2cc(Br)sc12]

To a mixture of N-(5-amino-2-methylpyridin-3-yl)-2-bromopyrazolo[5,1-b]thiazole-7-carboxamide (61 mg, 0.16 mmol), 2-(1-(tert-butoxycarbonyl)azetidin-3-yl)acetic acid (39 mg, 0.18 mmol), and HATU (73 mg, 0.19 mmol) in a 4 mL vial was added DMF (2 mL) with stirring to yield a clear pale yellow solution. TEA (86 μL, 0.62 mmol) was added and the solution became cloudy and brighter yellow. After 35 min the reaction was filtered and purified by prep-HPLC, 28%-48% MeCN/water/10 mM NH₄OH to yield tert-butyl 3-(2-((5-(2-bromopyrazolo[5,1-b]thiazole-7-carboxamido)-6-methylpyridin-3-yl)amino)-2-oxoethyl)azetidine-1-carboxylate (69 mg, 81%) as a white solid. LCMS (ESI): mass calcd. for C₂₂H₂₅BrN₆O₄S, 549.4; m/z found, 571.2/573.1 [M+Na].

### Step b: tert-butyl 3-(2-((5-(2-(5,6-dihydro-4H-pyrrolo[1,2-b]pyrazol-3-yl)pyrazolo[5,1-b]thiazole-7-carboxamido)-6-methylpyridin-3-yl)amino)-2-oxoethyl)azetidine-1-carboxylate

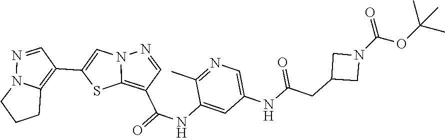 [SMILES: Cc1ncc(NC(=O)CC2CN(C(=O)OC(C)(C)C)C2)cc1NC(=O)c1cnn2cc(-c3cnn4c3CCC4)sc12]

The title compound was prepared from tert-butyl 3-(2-((5-(2-bromopyrazolo[5,1-b]thiazole-7-carboxamido)-6-methylpyridin-3-yl)amino)-2-oxoethyl)azetidine-1-carboxylate according to the procedure of Example 372, substituting 3-(tetramethyl-1,3,2-dioxaborolan-2-yl)-4H,5H,6H-pyrrolo[1,2-b]pyrazole for 3-fluoro-5-(4,4,5,5-tetramethyl-1,3,2-dioxaborolan-2-yl)pyridin-2-amine (34 mg, 47%). LCMS (ESI): mass calcd. for C₂₈H₃₂N₈O₄S, 576.7; m/z found, 599.2 [M+Na].

### Step c: N-(5-(2-(azetidin-3-yl)acetamido)-2-methylpyridin-3-yl)-2-(5,6-dihydro-4H-pyrrolo[1,2-b]pyrazol-3-yl)pyrazolo[5,1-b]thiazole-7-carboxamide

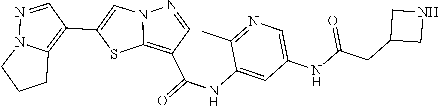 [SMILES: Cc1ncc(NC(=O)CC2CNC2)cc1NC(=O)c1cnn2cc(-c3cnn4c3CCC4)sc12]

To a clear solution of tert-butyl 3-(2-((5-(2-(5,6-dihydro-4H-pyrrolo[1,2-b]pyrazol-3-yl)pyrazolo[5,1-b]thiazole-7-carboxamido)-6-methylpyridin-3-yl)amino)-2-oxoethyl)azetidine-1-carboxylate (34 mg, 0.059 mmol) in DCM (1 mL) was added TFA (0.5 mL) and the reaction was allowed to stand at rt for 45 min then concentrated and purified by prep-HPLC, 10%-30% MeCN/water/0.1% TFA to yield N-(5-(2-(azetidin-3-yl)acetamido)-2-methylpyridin-3-yl)-2-(5,6-dihydro-4H-pyrrolo[1,2-b]pyrazol-3-yl)pyrazolo[5,1-b]thiazole-7-carboxamide (38 mg, 109%) as a pale yellow solid. LCMS (ESI): mass calcd. for C₂₃H₂₄N₈O₂S, 476.6; m/z found, 477.1 [M+H]⁺. ¹H NMR (METHANOL-d4) δ: 8.92 (d, J=2.0 Hz, 1H), 8.56 (d, J=2.4 Hz, 1H), 8.41 (s, 1H), 8.18 (s, 1H), 7.83 (s, 1H), 4.15-4.26 (m, 4H), 3.97-4.06 (m, 2H), 3.34-3.41 (m, 1H), 3.08-3.16 (m, 2H), 2.90 (d, J=7.3 Hz, 2H), 2.70-2.79 (m, 2H), 2.65 (s, 3H).

### Example 427. 2-(5,6-dihydro-4H-pyrrolo[1,2-b]pyrazol-3-yl)-N-(2-methyl-5-(2-(1-methylazetidin-3-yl)acetamido)pyridin-3-yl)pyrazolo[5,1-b]thiazole-7-carboxamide

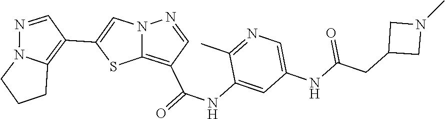 [SMILES: Cc1ncc(NC(=O)CC2CN(C)C2)cc1NC(=O)c1cnn2cc(-c3cnn4c3CCC4)sc12]

To a pale yellow solution of N-(5-(2-(azetidin-3-yl)acetamido)-2-methylpyridin-3-yl)-2-(5,6-dihydro-4H-pyrrolo[1,2-b]pyrazol-3-yl)pyrazolo[5,1-b]thiazole-7-carboxamide (24 mg, 0.041 mmol) and formalin (5 μL, 0.061 mmol) in MeOH (1.5 mL) was added NaBH(OAc)₃ (11 mg, 0.053 mmol) and the reaction was stirred at rt for 30 min. Excess formalin and NaBH(OAc)₃ were added and the reaction was stirred over the weekend. The reaction was concentrated, partitioned between EtOAc/sat'd NaHCO₃, and filtered of a small amount of a gummy white solid which was dissolved in DMF and purified by prep-HPLC, 29%-49% MeCN/water/10 mM NH₄OH to yield 2-(5,6-dihydro-4H-pyrrolo[1,2-b]pyrazol-3-yl)-N-(2-methyl-5-(2-(1-methylazetidin-3-yl)acetamido)pyridin-3-yl)pyrazolo[5,1-b]thiazole-7-carboxamide

(3.2 mg, 16%) as a white solid. LCMS (ESI): mass calcd. for C₂₄H₂₆N₈O₂S, 490.6; m/z found, 491.2 [M+H]⁺. ¹H NMR (METHANOL-d4) δ: 8.52 (d, J=2.4 Hz, 1H), 8.37 (s, 1H), 8.20 (d, J=2.4 Hz, 1H), 8.13 (s, 1H), 7.82 (s, 1H), 4.18 (t, J=7.3 Hz, 2H), 3.55 (t, J=7.8 Hz, 2H), 3.07-3.14 (m, 2H), 3.03 (dd, J=7.8, 6.8 Hz, 2H), 2.88 (dquin, J=14.3, 7.2 Hz, 1H), 2.71-2.79 (m, 2H), 2.68 (d, J=7.8 Hz, 2H), 2.48 (s, 3H), 2.33 (s, 3H).

### Example 428. 2-(1-(difluoromethyl)-1H-pyrazol-4-yl)-N-(5-(2-(3,3-dimethylazetidin-1-yl)acetamido)-2-methylpyridin-3-yl)pyrazolo[5,1-b]thiazole-7-carboxamide

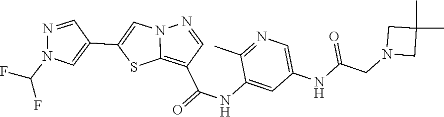 [SMILES: Cc1ncc(NC(=O)CN2CC(C)(C)C2)cc1NC(=O)c1cnn2cc(-c3cnn(C(F)F)c3)sc12]

A mixture of 3,5-dimethyl-1-(oxetan-3-yl)-4-(4,4,5,5-tetramethyl-1,3,2-dioxaborolan-2-yl)-1H-pyrazole (130 mg, 0.27 mmol), 1-(difluoromethyl)-4-(4,4,5,5-tetramethyl-1,3,2-dioxaborolan-2-yl)-1H-pyrazole (133 mg, 0.54 mmol), and cesium carbonate (266 mg, 0.82 mmol) in 1,4-dioxane (2.5 mL) and water (0.5 mL) was degassed for 20 min, treated with Pd DPPF G4 (26 mg, 0.027 mmol), and heated at 90° C. for 12 h. The reaction was cooled, concentrated, diluted with DMF (1.5 mL), and purified by prep-HPLC to yield 2-(1-(difluoromethyl)-1H-pyrazol-4-yl)-N-(5-(2-(3,3-dimethylazetidin-1-yl)acetamido)-2-methylpyridin-3-yl)pyrazolo[5,1-b]thiazole-7-carboxamide (16 mg, 11%). LCMS (ESI): mass calcd. for C₂₃H₂₄F₂N₈O₂S, 514.6; m/z found, 515.2 [M+H]+. ¹H NMR (METHANOL-d4) δ: 8.73 (d, J=2.4 Hz, 1H), 8.56 (s, 1H), 8.46 (s, 1H), 8.42-8.45 (m, 2H), 8.12 (s, 1H), 4.35 (s, 2H), 3.95-4.21 (m, 4H), 2.61 (s, 3H), 1.33-1.54 (m, 6H).

### Example 429. N-(5-(2-(3,3-dimethylazetidin-1-yl)acetamido)-2-methylpyridin-3-yl)-2-(pyrimidin-5-yl)pyrazolo[5,1-b]thiazole-7-carboxamide

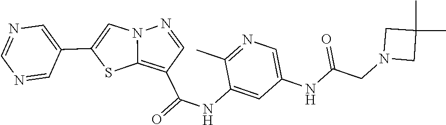 [SMILES: Cc1ncc(NC(=O)CN2CC(C)(C)C2)cc1NC(=O)c1cnn2cc(-c3cncnc3)sc12]

The title compound was prepared from 3,5-dimethyl-1-(oxetan-3-yl)-4-(4,4,5,5-tetramethyl-1,3,2-dioxaborolan-2-yl)-1H-pyrazole according to the procedure of Example 428, replacing 1-(difluoromethyl)-4-(4,4,5,5-tetramethyl-1,3,2-dioxaborolan-2-yl)-1H-pyrazole with 5-(4,4,5,5-tetramethyl-1,3,2-dioxaborolan-2-yl)pyrimidine (7 mg, 7%). LCMS (ESI): mass calcd. for C₂₃H₂₄N₈O₂S, 476.6; m/z found, 477.2 [M+H]⁺. ¹H NMR (400 MHz, METHANOL-d4) δ ppm 1.26 (t, J=7.09 Hz, 1H) 1.29 (s, 7H) 1.93 (s, 1H) 2.03 (s, 1H) 2.52 (s, 4H) 3.15 (s, 1H) 3.22-3.28 (m, 6H) 3.36-3.42 (m, 3H) 3.50 (s, 1H) 4.60 (s, 2H) 4.80-4.80 (m, 1H) 4.95 (s, 1H) 8.26 (d, J=2.45 Hz, 1H) 8.50 (s, 1H) 8.59 (d, J=2.45 Hz, 1H) 8.81 (s, 1H) 9.16-9.20 (m, 3H).

### Example 430. 2-(1,3-Dimethyl-1H-pyrazol-4-yl)-N-(5-(2-(3,3-dimethylazetidin-1-yl)acetamido)-2-methylpyridin-3-yl)pyrazolo[5,1-b]thiazole-7-carboxamide

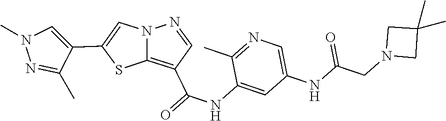

2-Bromo-N-(5-(2-(3,3-dimethylazetidin-1-yl)acetamido)-2-methylpyridin-3-yl)pyrazolo[5,1-b]thiazole-7-carboxamide (51.8 mg, 0.0814 mmol), 1,3-dimethyl-1H-pyrazole-4-boronic acid, pinacol ester (59.0 mg, 0.266 mmol), cesium carbonate (167 mg, 0.511 mmol), and 1,4-dioxane:distilled water (5:1) (3 mL) were combined in a 2-5 mL microwave vial, equipped with a stir bar. The contents were sparged with nitrogen with vigorous stirring. 1,1′-Bis(diphenylphosphino)ferrocene-palladium(II)dichloride dichloromethane complex (20.8 mg, 0.0255 mmol) was added. The reaction was irradiated in a Biotage Initiator+microwave reactor at 130° C. for 1 h and allowed to cool to room temperature. Solvent was removed under reduced pressure. The reaction contents were taken up in a minimal amount of MeOH and loaded on a neutral alumina cartridge. Alumina gel chromatography [neutral] was used to remove major impurities with using a 100% EtOAc to 10% MeOH/EtOAc. The combined fractions were concentrated, filtered, and purified by preparative high-performance liquid chromatography over column: Waters XBridge BEH C18 5 μm to afford 2-(1,3-Dimethyl-1H-pyrazol-4-yl)-N-(5-(2-(3,3-dimethylazetidin-1-yl)acetamido)-2-methylpyridin-3-yl)pyrazolo[5,1-b]thiazole-7-carboxamide as white solid (11 mg, 28%). LCMS (ESI): mass calcd for C₂₄H₂₈N₈O₂S, 492.2; m/z found, 493.2 [M+H]⁺. ¹H NMR (CHLOROFORM-d, 400 MHz) δ 9.03 (s, 1H), 8.64 (d, 1H, J=2.2 Hz), 8.51 (d, 1H, J=2.2 Hz), 8.08 (s, 1H), 7.75 (s, 1H), 7.52 (s, 1H), 7.43 (s, 1H), 3.90 (s, 3H), 3.27 (s, 2H), 3.16 (s, 4H), 2.56 (s, 3H), 2.42 (s, 3H), 1.28 (s, 6H).

### Example 431. 2-(1,3-Dimethyl-1H-pyrazol-4-yl)-N-(5-(2-trans-2,6-dimethylmorpholino)acetamido)-2-methylpyridin-3-yl)pyrazolo[5,1-b]thiazole-7-carboxamide

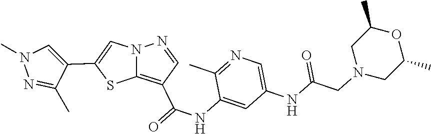

2-Bromo-N-(5-(2-trans-2,6-dimethylmorpholino)acetamido)-2-methylpyridin-3-yl)pyrazolo[5,1-b]thiazole-7-carboxamide (122 mg, 0.240 mmol), 1,3-dimethyl-1H-pyrazole-4-boronic acid pinacol ester (133 mg, 0.600 mmol), cesium carbonate (249 mg, 0.762 mmol), and 1,4-dioxane:distilled water (5:1) (3.5 mL) were combined in a 2-5 mL microwave vial, equipped with a stir bar. The contents were sparged with nitrogen with vigorous stirring. 1,1′-Bis(diphenylphosphino)ferrocene-palladium(II)dichloride dichloromethane complex (21.3 mg, 0.0261 mmol) was added. The vial was sealed with a cap. The reaction was irradiated in a Biotage Initiator+microwave reactor at 130° C. for two one-hour increments. Each time, the reaction was allowed to cool to room temperature. Additional 1,3-dimethyl-1H-pyrazole-4-boronic acid pinacol ester (57.6 mg, 0.259 mmol), cesium carbonate (62.5 mg, 0.192 mmol), and 1,1′-bis(diphenylphosphino)ferrocene-palladium(II)dichloride dichloromethane complex (8.6 mg, 0.0105 mmol) were added to the reaction pot. The reaction was irradiated again for 1 h and allowed to cool to room temperature. More cesium carbonate (142 mg, 0.436), 1,3-dimethyl-1H-pyrazole-4-boronic acid pinacol ester (98.0 mg, 0.253 mmol), and 1,1′-bis(diphenylphosphino)ferrocene-palladium(II)dichloride dichloromethane complex (14.5 mg, 0.0178 mmol) were added. The reaction was irradiated for 1 h and allowed to cool to room temperature. The mixture was filtered through Si-Tris amine metal scavenger and concentrated. The crude residue was purified by preparative high-performance liquid chromatography over column: Kinetex 5 μm EVO C18 100 Å [Water 10 mM (NH₄)₂CO₃/0.1% NH₄OH] [10% Water/90% ACN 10 mM (NH₄)₂CO₃/0.1% NH₄OH] to afford 2-(1,3-dimethyl-1H-pyrazol-4-yl)-N-(5-(2-trans-2,6-dimethylmorpholino)acetamido)-2-methylpyridin-3-yl)pyrazolo[5,1-b]thiazole-7-carboxamide as a white solid (51 mg, 39%). LCMS (ESI): mass calcd for C₂₅H₃₀N₈O₃S, 522.2; m/z found, 523.2 [M+H]⁺. ¹H NMR (CHLOROFORM-d, 400 MHz) δ 9.10 (s, 1H), 8.61 (d, 1H, J=2.4 Hz), 8.56 (d, 1H, J=2.2 Hz), 8.07 (s, 1H), 7.77 (s, 1H), 7.53 (s, 1H), 7.38 (s, 1H), 4.1-4.2 (m, 2H), 3.91 (s, 3H), 3.85 (s, 1H), 3.1-3.2 (m, 1H), 3.0-3.1 (m, 1H), 2.66 (dd, 2H, J=3.1, 11.1 Hz), 2.59 (s, 3H), 2.43 (s, 3H), 2.3-2.4 (m, 2H), 1.34 (d, 5H, J=6.4 Hz).

### Example 432. N-(5-(2-(trans-2,6-dimethylmorpholino)acetamido)-2-methylpyridin-3-yl)-2-(1-(2-(methylsulfonyl)ethyl)-1H-pyrazol-4-yl)pyrazolo[5,1-b]thiazole-7-carboxamide

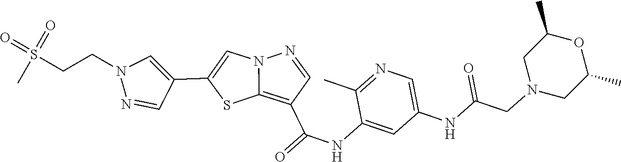 [SMILES: Cc1ncc(NC(=O)CN2C[C@@H](C)O[C@H](C)C2)cc1NC(=O)c1cnn2cc(-c3cnn(CCS(C)(=O)=O)c3)sc12]

2-Bromo-N-(5-(2-trans-2,6-dimethylmorpholino)acetamido)-2-methylpyridin-3-yl)pyrazolo[5,1-b]thiazole-7-carboxamide (115 mg, 0.227 mmol), 1-(2-methanesulfonylethyl)-4-(tetramethyl-1,3,2-dioxaborolan-2-yl)-1H-pyrazole (149 mg, 0.498 mmol), cesium carbonate (224 mg, 0.687 mmol), and 1,4-dioxane:distilled water (5:1) (4 mL) were combined in a 2-5 mL microwave vial, equipped with a stir bar. The contents were sparged with nitrogen with vigorous stirring. 1,1′-Bis(diphenylphosphino)ferrocene-palladium(II)dichloride dichloromethane complex (21.2 mg, 0.026 mmol) was added. The vial was sealed with a cap. The reaction was irradiated in a Biotage Initiator+microwave reactor at 130° C. for 1 h and allowed to cool to room temperature.

Additional 1-(2-methanesulfonylethyl)-4-(tetramethyl-1,3,2-dioxaborolan-2-yl)-1H-pyrazole (32.2 mg, 0.107 mmol) was added. The reaction was heated for another hour at 130° C. and allowed to cool to room temperature. Cesium carbonate (100 mg, 0.307 mmol), 1-(2-methanesulfonylethyl)-4-(tetramethyl-1,3,2-dioxaborolan-2-yl)-1H-pyrazole (37.4 mg, 0.124 mmol) and 1,1′-bis(diphenylphosphino)ferrocene-palladium(II)dichloride dichloromethane complex (10.2 mg, 0.0125) were added to the vial. The mixture was heated at 130° C. and allowed to cool to room temperature. The mixture was filtered through Si-Tris amine metal scavenger and concentrated. The crude residue was purified by preparative high-performance liquid chromatography over column: Waters XBridge BEH C18 5 μm to afford N-(5-(2-trans-2,6-dimethylmorpholino)acetamido)-2-methylpyridin-3-yl)-2-(1-(2-(methylsulfonyl)ethyl)-1H-pyrazol-4-yl)pyrazolo[5,1-b]thiazole-7-carboxamide as a light brown solid (26 mg, 18%). LCMS (ESI): mass calcd for C₂₆H₃₂N₈O₅S₂, 600.2; m/z found, 601.1 [M+H]+. ¹H NMR (DMSO-d6, 400 MHz) δ 9.88 (d, 2H, J=8.8 Hz), 8.62 (s, 1H), 8.54 (d, 1H, J=2.4 Hz), 8.51 (s, 1H), 8.32 (d, 1H, J=0.7 Hz), 8.16 (d, 1H, J=2.2 Hz), 7.98 (d, 1H, J=0.7 Hz), 4.59 (t, 2H, J=7.0 Hz), 3.9-4.0 (m, 2H), 3.74 (t, 2H, J=6.8 Hz), 3.1-3.2 (m, 1H), 3.0-3.1 (m, 1H), 2.95 (s, 3H), 2.54 (dd, 2H, J=3.2, 11.0 Hz), 2.40 (s, 3H), 2.21 (dd, 2H, J=5.7, 10.9 Hz), 1.18 (d, 6H, J=6.6 Hz).

### Example 433. 2-(1,5-Dimethyl-1H-pyrazol-4-yl)-N-(5-(2-(trans-2,6-dimethylmorpholino)acetamido)-2-methylpyridin-3-yl)pyrazolo[5,1-b]thiazole-7-carboxamide

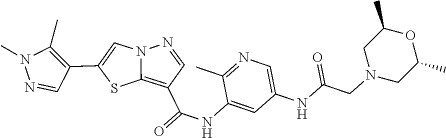

2-Bromo-N-(5-(2-trans-2,6-dimethylmorpholino)acetamido)-2-methylpyridin-3-yl)pyrazolo[5,1-b]thiazole-7-carboxamide (124 mg, 0.243 mmol), 1,5-dimethyl-4-(4,4,5,5-tetramethyl-1,3,2-dioxaborolan-2-yl)-1H-pyrazole (136 mg, 0.614 mmol), cesium carbonate (249 mg, 0.764 mmol), and 1,4-dioxane:distilled water (5:1) (5 mL) were combined in a 2-5 mL microwave vial, equipped with a stir bar. The contents were sparged with nitrogen with vigorous stirring. 1,1′-Bis(diphenylphosphino)ferrocene-palladium(II)dichloride dichloromethane complex (21.3 mg, 0.0261 mmol) was added, and the vial was sealed with a cap. The reaction was irradiated in a Biotage Initiator+microwave reactor at 130° C. for two one-hour increments with cooling after each hour. Additional 1,5-dimethyl-4-(4,4,5,5-tetramethyl-1,3,2-dioxaborolan-2-yl)-1H-pyrazole (98 mg, 0.441 mmol), cesium carbonate (114 mg, 0.350 mmol), and 1,1′-bis(diphenylphosphino)ferrocene-palladium(II)dichloride dichloromethane complex (14.9 mg, 0.0182 mmol) were added. The mixture was irradiated for 1 h and allowed to cool to room temperature. Solvent was removed under reduced pressure. The residue was taken up in MeOH and loaded on a neutral alumina cartridge. Alumina gel chromatography [neutral] was used to further purify the crude residue with a 100% EtOAc to 10% MeOH/EtOAc gradient. The combined fractions were concentrated, filtered, and purified by preparative high-performance liquid chromatography over column: Waters XBridge BEH C18 5 μm to give 2-(1,5-dimethyl-1H-pyrazol-4-yl)-N-(5-(2-trans-2,6-dimethylmorpholino)acetamido)-2-methylpyridin-3-yl)pyrazolo[5,1-b]thiazole-7-carboxamide as a white solid (44 mg, 34%). LCMS (ESI): mass calcd for C₂₅H₃₀N₈O₃S, 522.2; m/z found, 523.2 [M+H]+. ¹H NMR (DMSO-d6, 400 MHz) δ 9.87 (d, 2H, J=5.6 Hz), 8.5-8.6 (m, 2H), 8.45 (s, 1H), 8.17 (d, 1H, J=2.2 Hz), 7.71 (s, 1H), 3.9-4.0 (m, 2H), 3.80 (s, 3H), 3.1-3.2 (m, 1H), 3.0-3.1 (m, 1H), 2.54 (dd, 2H, J=2.9, 11.0 Hz), 2.44 (s, 3H), 2.40 (s, 3H), 2.21 (dd, 2H, J=5.5, 10.9 Hz), 1.18 (d, 6H, J=6.4 Hz).

### Example 434. 2-(1,5-Dimethyl-1H-pyrazol-4-yl)-N-(5-(2-(3,3-dimethylazetidin-1-yl)acetamido)-2-methylpyridin-3-yl)pyrazolo[5,1-b]thiazole-7-carboxamide

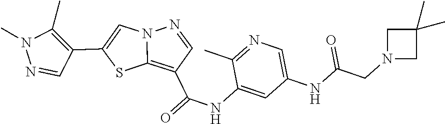

2-Bromo-N-(5-(2-(3,3-dimethylazetidin-1-yl)acetamido)-2-methylpyridin-3-yl)pyrazolo[5,1-b]thiazole-7-carboxamide (81.9 mg, 0.129 mmol), 1,5-dimethyl-4-(4,4,5,5-tetramethyl-1,3,2-dioxaborolan-2-yl)-1 h-pyrazole (145.2 mg, 0.654 mmol), cesium carbonate (277 mg, 0.849 mmol), 1,1′-bis(diphenylphosphino)ferrocene-palladium(II)dichloride dichloromethane complex (22.3 mg, 0.0273 mmol), and 1,4-dioxane:distilled water (5:1) (3 mL) were combined in a 2-5 mL microwave vial, equipped with a stir bar. The contents were sparged with nitrogen with vigorous stirring. The mixture was irradiated in a Biotage Initiator+microwave reactor at 130° C. for two one-hour increments and allowed to cool to room temperature each time. Additional components were added 1,5-dimethyl-4-(4,4,5,5-tetramethyl-1,3,2-dioxaborolan-2-yl)-1H-pyrazole (114 mg, 0.511), cesium carbonate (181 mg, 0.554 mmol), and 1,1′-bis(diphenylphosphino)ferrocene-palladium(ii)dichloride dichloromethane complex (14.7 mg, 0.0180 mmol). The mixture was irradiated 130° C. for 1 h and allowed to cool to room temperature. The mixture was filtered through Si-Tris amine metal scavenger and concentrated. The crude residue was purified by preparative high-performance liquid chromatography over column: Waters XBridge BEH C18 5 μm to afford 2-(1,5-dimethyl-1H-pyrazol-4-yl)-N-(5-(2-(3,3-dimethylazetidin-1-yl)acetamido)-2-methylpyridin-3-yl)pyrazolo[5,1-b]thiazole-7-carboxamide as a white solid (8.9 mg, 14%). LCMS (ESI): mass calcd for C₂₄H₂₈N₈O₂S, 492.2; m/z found, 493.1 [M+H]+. ¹H NMR (CHLOROFORM-d, 400 MHz) δ 9.17 (br s, 1H), 8.62 (s, 1H), 8.48 (s, 1H), 8.11 (s, 1H), 7.73 (s, 1H), 7.58 (s, 1H), 7.52 (br s, 1H), 3.87 (s, 3H), 3.33 (s, 2H), 3.23 (s, 4H), 2.56 (s, 3H), 2.44 (s, 3H), 1.29 (s, 6H).

### Example 435. N-(5-(2-(3,3-Dimethylazetidin-1-yl)acetamido)-2-methylpyridin-3-yl)-2-(1-(2-(methylsulfonyl)ethyl)-1H-pyrazol-4-yl)pyrazolo[5,1-b]thiazole-7-carboxamide

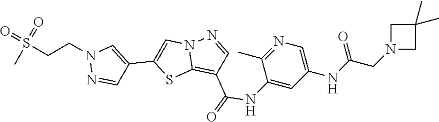 [SMILES: Cc1ncc(NC(=O)CN2CC(C)(C)C2)cc1NC(=O)c1cnn2cc(-c3cnn(CCS(C)(=O)=O)c3)sc12]

2-Bromo-N-(5-(2-(3,3-dimethylazetidin-1-yl)acetamido)-2-methylpyridin-3-yl)pyrazolo[5,1-b]thiazole-7-carboxamide (86.2 mg, 0.164 mmol), 1-(2-methanesulfonylethyl)-4-(tetramethyl-1,3,2-dioxaborolan-2-yl)-1H-pyrazole (199 mg, 0.663 mmol), cesium carbonate (266 mg, 0.809 mmol), 1,1′-bis(diphenylphosphino)ferrocene-palladium(II)dichloride dichloromethane complex (30.0 mg, 0.0368 mmol), and 1,4-dioxane:distilled water (5:1) (3.5 mL) were combined in a 2-5 mL microwave vial, equipped with a stir bar. The contents were sparged with nitrogen with vigorous stirring. The reaction was irradiated in a Biotage Initiator+microwave reactor at 130° C. for 1 h and allowed to cool to room temperature. Solvent was removed under reduced pressure. The crude residue was taken up in MeOH and passed through Si-Trisamine metal scavenger. Solvent was removed under reduced pressure. The crude residue was purified by preparative high-performance liquid chromatography over column: Waters XBridge BEH C18 5 μm to provide N-(5-(2-(3,3-Dimethylazetidin-1-yl)acetamido)-2-methylpyridin-3-yl)-2-(1-(2-(methylsulfonyl)ethyl)-1H-pyrazol-4-yl)pyrazolo[5,1-b]thiazole-7-carboxamide as an off-white solid (25 mg, 25%). LCMS (ESI): mass calcd for C₂₅H₃₀N₈O₄S₂, 570.2; m/z found, 571.0 [M+H]+. ¹H NMR (DMSO-d6, 400 MHz) (3.3:1.0 Rotameric mixture at 25° C.) δ 9.9-9.9 (m, 1.93H), 8.62 (s, 0.96H), 8.56 (d, 1.02H, J=2.2 Hz), 8.51 (s, 0.96H), 8.32 (s, 0.97H), 8.14 (s, 1.48H), 8.0-8.0 (m, 1.25H), 7.69 (d, 0.26H, J=0.7 Hz) (may be impurity), 4.59 (t, 2.11H, J=6.8 Hz), 4.52 (t, 0.63H, J=6.8 Hz), 3.74 (t, 2.22H, J=7.0 Hz), 3.70 (t, 0.70H, J=7.0 Hz), 3.3-3.3 (m, 2.58H), 3.10 (s, 4.22H), 2.95 (s, 3.10H), 2.86 (s, 0.88H), 2.40 (s, 3.06H), 1.21 (s, 6.40H).

### Example 436. N-(5-(2-(3,3-Dimethylazetidin-1-yl)acetamido)-2-methylpyridin-3-yl)-2-(2-ethoxypyridin-3-yl)pyrazolo[5,1-b]thiazole-7-carboxamide

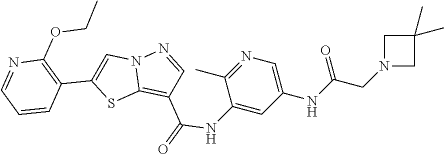 [SMILES: CCOc1ncccc1-c1cn2ncc(C(=O)Nc3cc(NC(=O)CN4CC(C)(C)C4)cnc3C)c2s1]

2-Bromo-N-(5-(2-(3,3-dimethylazetidin-1-yl)acetamido)-2-methylpyridin-3-yl)pyrazolo[5,1-b]thiazole-7-carboxamide (87 mg, 0.166 mmol), 2-ethoxypyridine-3-boronic acid (116 mg, 0.679 mmol), cesium carbonate (267 mg, 0.811 mmol), 1,1′-bis(diphenylphosphino)ferrocene-palladium(II)dichloride dichloromethane complex (28.9 mg, 0.0354 mmol), and 1,4-dioxane:distilled water (5:1) (3.5 mL) were combined in a 2-5 mL microwave vial, equipped with a stir bar. The contents were sparged with nitrogen with vigorous stirring. The reaction was irradiated in a Biotage Initiator+microwave reactor at 130° C. for two one-hour intervals and allowed to cool to room temperature each time. Solvent was removed under reduced pressure. The residue was taken up in a minimal amount of MeOH and loaded on a neutral alumina cartridge. Alumina gel chromatography [neutral] was used to further purify the crude residue with a 100% EtOAc to 10% MeOH/EtOAc gradient. The combined fractions were concentrated, filtered, and purified by preparative high-performance liquid chromatography over column: Waters XBridge BEH C18 5 μm to give N-(5-(2-(3,3-dimethylazetidin-1-yl)acetamido)-2-methylpyridin-3-yl)-2-(2-ethoxypyridin-3-yl)pyrazolo[5,1-b]thiazole-7-carboxamide as a white solid (23 mg, 24%). LCMS (ESI): mass calcd for C₂₆H₂₉N₇O₃S, 519.2; m/z found, 520.2 [M+H]⁺. ¹H NMR (CHLOROFORM-d, 400 MHz) δ 9.11 (s, 1H), 8.64 (d, 1H, J=2.4 Hz), 8.54 (d, 1H, J=2.4 Hz), 8.47 (s, 1H), 8.17 (dd, 1H, J=1.7, 4.9 Hz), 8.13 (s, 1H), 7.79 (dd, 1H, J=1.7, 7.6 Hz), 7.43 (s, 1H), 7.00 (dd, 1H, J=5.0, 7.5 Hz), 4.58 (q, 2H, J=7.1 Hz), 3.30 (s, 2H), 3.19 (s, 4H), 2.58 (s, 3H), 1.53 (t, 3H, J=7.1 Hz), 1.29 (s, 6H).

### Example 437. N-(5-(2-(3,3-dimethylazetidin-1-yl)acetamido)-2-methylpyridin-3-yl)-2-(2-(trifluoromethoxy)pyridin-3-yl)pyrazolo[5,1-b]thiazole-7-carboxamide

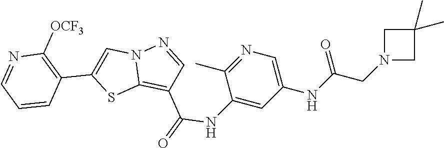 [SMILES: Cc1ncc(NC(=O)CN2CC(C)(C)C2)cc1NC(=O)c1cnn2cc(-c3cccnc3OC(F)(F)F)sc12]

2-Bromo-N-(5-(2-(3,3-dimethylazetidin-1-yl)acetamido)-2-methylpyridin-3-yl)pyrazolo[5,1-b]thiazole-7-carboxamide (60.3 mg, 0.115 mmol), 3-(4,4,5,5-tetramethyl-1,3,2-dioxaborolan-2-yl)-2-(trifluoromethoxy)pyridine (82.0 mg, 0.269 mmol), cesium carbonate (126 mg, 0.382 mmol), 1,1′-bis(diphenylphosphino)ferrocene-palladium(II)dichloride dichloromethane complex (24.6 mg, 0.0301 mmol), and 1,4-dioxane:distilled water (5:1) (2.5 mL) were combined in a 2-5 mL microwave vial, equipped with a stir bar. The contents were sparged with nitrogen with vigorous stirring. The vial was sealed with a cap. The reaction was irradiated in a Biotage Initiator+microwave reactor at 110° C. for 1 h and allowed to cool to room temperature. Solvent was removed under reduced pressure. The residue was taken up in a minimal amount of MeOH and loaded on a neutral alumina cartridge. Alumina gel chromatography [neutral] was used to further purify the crude residue with a 100% EtOAc to 10% MeOH/EtOAc gradient. The combined fractions were concentrated, filtered, and purified by preparative high-performance liquid chromatography over column: Waters XBridge BEH C18 5 μm to afford N-(5-(2-(3,3-dimethylazetidin-1-yl)acetamido)-2-methylpyridin-3-yl)-2-(2-(trifluoromethoxy)pyridin-3-yl)pyrazolo[5,1-b]thiazole-7-carboxamide as an off-white solid (12 mg, 18%). LCMS (ESI): mass calcd for C₂₅H₂₄F₃N₇O₃S, 559.2; m/z found, 560.1 [M+H]+. ¹H NMR (CHLOROFORM-d, 400 MHz) δ 9.12 (s, 1H), 8.63 (d, 1H, J=2.4 Hz), 8.56 (d, 1H, J=2.4 Hz), 8.36 (s, 1H), 8.33 (dd, 1H, J=1.7, 4.9 Hz), 8.14 (s, 1H), 7.96 (dd, 1H, J=1.8, 7.7 Hz), 7.3-7.4 (m, 2H), 3.29 (s, 2H), 3.19 (s, 4H), 2.58 (s, 3H), 1.29 (s, 6H).

### Example 438. (S)-2-(1,3-dimethyl-1H-pyrazol-4-yl)-N-(2-methyl-5-(2-(2-methylpyrrolidin-1-yl)acetamido)pyridin-3-yl)pyrazolo[5,1-b]thiazole-7-carboxamide

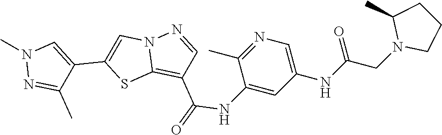 [SMILES: Cc1ncc(NC(=O)CN2CCC[C@@H]2C)cc1NC(=O)c1cnn2cc(-c3cn(C)nc3C)sc12]

### Step a: (S)-2-bromo-N-(2-methyl-5-(2-(2-methylpyrrolidin-1-yl)acetamido)pyridin-3-yl)pyrazolo[5,1-b]thiazole-7-carboxamide

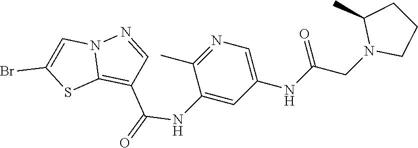 [SMILES: Cc1ncc(NC(=O)CN2CCC[C@@H]2C)cc1NC(=O)c1cnn2cc(Br)sc12]

A 30 mL vial, equipped with a stir bar, was charged with 2-bromo-N-(5-(2-chloroacetamido)-2-methylpyridin-3-yl)pyrazolo[5,1-b]thiazole-7-carboxamide (1.07 g, 2.26 mmol), K₂CO₃ (0.967 g, 7.00 mmol), DMF (10 mL), and (S)-2-methyl-pyrrolidine (0.4 mL, 3.92 mmol). A nitrogen atmosphere was established. The mixture was heated to 50° C. After approximately 7 h, the mixture was allowed to cool to 23° C. The mixture was poured into water (100 mL) and allowed to stir for 20 min. Precipitate was collected via filtration. The filter cake was washed with water. The solid was allowed to air-dry under vacuum overnight to provide (S)-2-bromo-N-(2-methyl-5-(2-(2-methylpyrrolidin-1-yl)acetamido)pyridin-3-yl)pyrazolo[5,1-b]thiazole-7-carboxamide as a light brown solid (526 mg, 48%). LCMS (ESI): mass calcd for C₁₉H₂₁BrN₆O₂S, 476.1; m/z found, 477.0 [M+H]+. ¹H NMR (CHLOROFORM-d, 400 MHz) δ (ppm) 9.22 (br s, 1H), 8.58 (d, 1H, J=2.0 Hz), 8.36 (d, 1H, J=2.4 Hz), 8.25 (s, 1H), 7.88 (s, 1H), 7.82 (s, 1H), 3.44 (d, 1H, J=16.6 Hz), 3.19 (dt, 1H, J=3.4, 8.6 Hz), 3.10 (d, 1H, J=17.1 Hz), 2.6-2.7 (m, 1H), 2.4-2.5 (m, 4H), 2.0-2.1 (m, 1H), 1.7-1.9 (m, 2H), 1.5-1.6 (m, 1H), 1.13 (d, 3H, J=5.9 Hz).

### Step b: (S)-2-(1,3-dimethyl-1H-pyrazol-4-yl)-N-(2-methyl-5-(2-(2-methylpyrrolidin-1-yl)acetamido)pyridin-3-yl)pyrazolo[5,1-b]thiazole-7-carboxamide

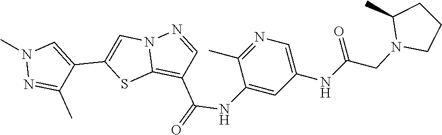 [SMILES: Cc1ncc(NC(=O)CN2CCC[C@@H]2C)cc1NC(=O)c1cnn2cc(-c3cn(C)nc3C)sc12]

(S)-2-Bromo-N-(2-methyl-5-(2-(2-methylpyrrolidin-1-yl)acetamido)pyridin-3-yl)pyrazolo[5,1-b]thiazole-7-carboxamide (107 mg, 0.222 mmol), 1,3-dimethyl-1H-pyrazole-4-boronic acid pinacol ester (125 mg, 0.563 mmol), cesium carbonate (221 mg, 0.678 mmol), 1,1′-bis(diphenylphosphino)ferrocene-palladium(II)dichloride dichloromethane complex (36.3 mg, 0.0445 mmol), and 1,4-dioxane:distilled water (5:1) (2.5 mL) were combined in a 2-5 mL microwave vial, equipped with a stir bar. The vial was sealed with a cap. The contents were sparged with nitrogen with vigorous stirring. The mixture was irradiated in a Biotage Initiator+microwave reactor at 130° C. for 1 h and allowed to cool to room temperature. Solvent was removed under reduced pressure. The crude residue was taken up in MeOH. Si-Trisamine, a metal scavenger, was added. The mixture was stirred at room temperature for approximately 21 h. The crude mixture was filtered using a Biotage phase separator. Solvent was removed under reduced pressure. The crude residue was purified by preparative high-performance liquid chromatography over column: Waters XBridge BEH C18 5 μm to yield (S)-2-(1,3-dimethyl-1H-pyrazol-4-yl)-N-(2-methyl-5-(2-(2-methylpyrrolidin-1-yl)acetamido)pyridin-3-yl)pyrazolo[5,1-b]thiazole-7-carboxamide as a light brown solid (35 mg, 32%). LCMS (ESI): mass calcd for C₂₄H₂₈N₈O₂S, 492.2; m/z found, 493.1 [M+H]+. ¹H NMR (DMSO-d6, 400 MHz) δ 9.8-9.9 (m, 2H), 8.60 (d, 1H, J=2.2 Hz), 8.52 (s, 1H), 8.39 (s, 1H), 8.17 (d, 1H, J=2.4 Hz), 8.08 (s, 1H), 3.80 (s, 3H), 3.4-3.5 (m, 1H), 3.0-3.2 (m, 2H), 2.55 (br s, 1H), 2.3-2.4 (m, 7H), 1.9-2.0 (m, 1H), 1.6-1.8 (m, 2H), 1.3-1.5 (m, 1H), 1.0-1.1 (m, 3H).

### Example 439. 2-(1,3-Dimethyl-1H-pyrazol-4-yl)-N-(5-(2-(3-methoxypyrrolidin-1-yl)acetamido)-2-methylpyridin-3-yl)pyrazolo[5,1-b]thiazole-7-carboxamide

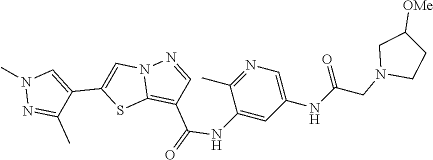 [SMILES: COC1CCN(CC(=O)Nc2cnc(C)c(NC(=O)c3cnn4cc(-c5cn(C)nc5C)sc34)c2)C1]

### Step a: 2-Bromo-N-(5-(2-(3-methoxypyrrolidin-1-yl)acetamido)-2-methylpyridin-3-yl)pyrazolo[5,1-b]thiazole-7-carboxamide

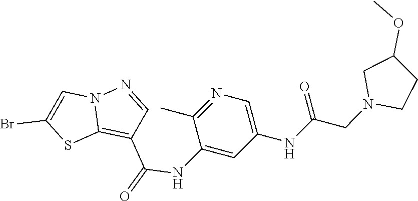 [SMILES: COC1CCN(CC(=O)Nc2cnc(C)c(NC(=O)c3cnn4cc(Br)sc34)c2)C1]

A 30 mL vial, equipped with a stir bar, was charged with 2-bromo-N-(5-(2-chloroacetamido)-2-methylpyridin-3-yl)pyrazolo[5,1-b]thiazole-7-carboxamide (0.600 g, 1.27 mmol), K₂CO₃ (0.526 g, 3.81 mmol), DMF (10 mL), and 3-methoxypyrrolidine (335 mg, 3.18 mmol). A nitrogen atmosphere was established. The mixture was heated to 50° C. After approximately 18 h, the mixture was allowed to cool to room temperature. The mixture was poured into water (150 mL) and allowed to stir for 22 min. Precipitate was collected via filtration. The filter cake was washed with water. The solid was allowed to air-dry under vacuum overnight to supply 2-bromo-N-(5-(2-(3-methoxypyrrolidin-1-yl)acetamido)-2-methylpyridin-3-yl)pyrazolo[5,1-b]thiazole-7-carboxamide as a light brown solid (230 mg, 37%). LCMS (ESI): mass calcd for C₁₉H₂₁BrN₆O₃S, 492.0; m/z found, 493.0 [M+H]+. ¹H NMR (DMSO-d6, 400 MHz) δ 9.95 (s, 2H), 8.78 (s, 1H), 8.60 (s, 1H), 8.57 (d, 1H, J=2.4 Hz), 8.16 (d, 1H, J=2.2 Hz), 3.9-4.0 (m, 1H), 3.27 (s, 2H), 3.18 (s, 3H), 2.89 (dd, 1H, J=6.4, 10.0 Hz), 2.5-2.7 (m, 3H), 2.39 (s, 3H), 2.0-2.1 (m, 1H), 1.7-1.8 (m, 1H).

### Step b: 2-(1,3-Dimethyl-1H-pyrazol-4-yl)-N-(5-(2-(3-methoxypyrrolidin-1-yl)acetamido)-2-methylpyridin-3-yl)pyrazolo[5,1-b]thiazole-7-carboxamide

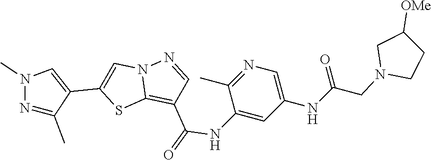 [SMILES: COC1CCN(CC(=O)Nc2cnc(C)c(NC(=O)c3cnn4cc(-c5cn(C)nc5C)sc34)c2)C1]

2-Bromo-N-(5-(2-(3-methoxypyrrolidin-1-yl)acetamido)-2-methylpyridin-3-yl)pyrazolo[5,1-b]thiazole-7-carboxamide (109 mg, 0.221 mmol), 1,3-dimethyl-1 h-pyrazole-4-boronic acid pinacol ester (121 mg, 0.543 mmol), cesium carbonate (218 mg, 0.668 mmol), 1,1′-bis(diphenylphosphino)ferrocene-palladium(II)dichloride dichloromethane complex (36.3 mg, 0.0445 mmol), and 1,4-dioxane:distilled water (5:1) (2.5 mL) were combined in a 2-5 mL microwave vial, equipped with a stir bar. The contents were sparged with nitrogen with vigorous stirring. The vial was sealed with a cap. The mixture was irradiated in a Biotage Initiator+microwave reactor at 130° C. for 1 h and allowed to cool to room temperature. Solvent was removed under reduced pressure. The crude residue was taken up in MeOH and Si-Trisamine, a metal scavenger. The mixture was stirred at room temperature for 14 h and 20 min. The crude mixture was filtered using a Biotage phase separator. The residue was purified by preparative high-performance liquid chromatography over column: Waters XBridge BEH C18 5 μm to afford 2-(1,3-dimethyl-1H-pyrazol-4-yl)-N-(5-(2-(3-methoxypyrrolidin-1-yl)acetamido)-2-methylpyridin-3-yl)pyrazolo[5,1-b]thiazole-7-carboxamide as a light brown solid (20 mg, 17%). LCMS (ESI): mass calcd for C₂₄H₂₈N₈O₃S, 508.2; m/z found, 509.2 [M+H]+. ¹H NMR (DMSO-d6, 400 MHz) S 9.94 (s, 1H), 9.87 (s, 1H), 8.56 (d, 1H, J=2.4 Hz), 8.51 (s, 1H), 8.39 (s, 1H), 8.17 (d, 1H, J=2.2 Hz), 8.08 (s, 1H), 3.9-4.0 (m, 1H), 3.80 (s, 3H), 3.27 (s, 2H), 3.18 (s, 3H), 2.89 (dd, 1H, J=6.2, 10.1 Hz), 2.5-2.7 (m, 3H), 2.40 (s, 3H), 2.34 (s, 3H), 2.0-2.1 (m, 1H), 1.7-1.8 (m, 1H).

### Example 440. (R)-2-(1,3-dimethyl-1H-pyrazol-4-yl)-N-(2-methyl-5-(2-(2-methylpyrrolidin-1-yl)acetamido)pyridin-3-yl)pyrazolo[5,1-b]thiazole-7-carboxamide

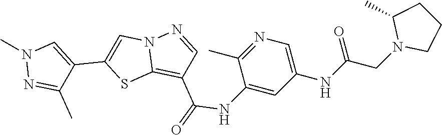 [SMILES: Cc1ncc(NC(=O)CN2CCC[C@H]2C)cc1NC(=O)c1cnn2cc(-c3cn(C)nc3C)sc12]

### Step a: (R)-2-bromo-N-(2-methyl-5-(2-(2-methylpyrrolidin-1-yl)acetamido)pyridin-3-yl)pyrazolo[5,1-b]thiazole-7-carboxamide

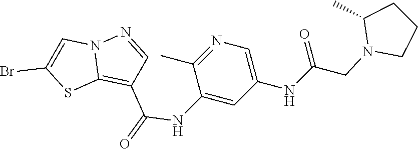 [SMILES: Cc1ncc(NC(=O)CN2CCC[C@H]2C)cc1NC(=O)c1cnn2cc(Br)sc12]

A 30 mL vial, equipped with a stir bar, was charged with 2-bromo-N-(5-(2-chloroacetamido)-2-methylpyridin-3-yl)pyrazolo[5,1-b]thiazole-7-carboxamide (1.08 g, 2.30 mmol), K₂CO₃ (0.946 g, 6.85 mmol), DMF (10 mL), and (R)-2-methylpyrrolidine (0.4 mL, 4 mmol). A nitrogen atmosphere was established. The mixture was heated to 50° C. After approximately 7 h, the mixture was allowed to cool to 23° C. The mixture was poured into water (100 mL) and allowed to stir for 20 min. Precipitate was collected via filtration. The filter cake was washed with water. The solid was allowed to air-dry under vacuum overnight to provide (R)-2-bromo-N-(2-methyl-5-(2-(2-methylpyrrolidin-1-yl)acetamido)pyridin-3-yl)pyrazolo[5,1-b]thiazole-7-carboxamide as a light brown solid (544 mg, 48%). LCMS (ESI): mass calcd for C₁₉H₂₁BrN₆O₂S, 476.1; m/z found, 477.0 [M+H]+. ¹H NMR (DMSO-d6, 400 MHz) δ 9.95 (s, 1H), 9.85 (s, 1H), 8.78 (s, 1H), 8.6-8.7 (m, 2H), 8.17 (d, 1H, J=2.4 Hz), 3.4-3.5 (m, 1H), 3.11 (dt, 1H, J=3.2, 8.7 Hz), 3.02 (d, 1H, J=15.7 Hz), 2.5-2.6 (m, 1H), 2.40 (s, 3H), 2.33 (q, 1H, J=8.6 Hz), 1.8-2.0 (m, 1H), 1.6-1.8 (m, 2H), 1.39 (dddd, 1H, J=6.4, 8.3, 10.3, 12.2 Hz), 1.07 (d, 3H, J=6.4 Hz).

### Step b: 2-(1,3-Dimethyl-1H-pyrazol-4-yl)-N-(5-(2-(3-methoxypyrrolidin-1-yl)acetamido)-2-methylpyridin-3-yl)pyrazolo[5,1-b]thiazole-7-carboxamide

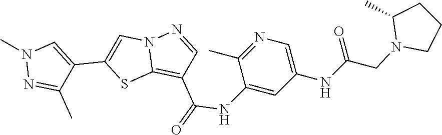 [SMILES: Cc1ncc(NC(=O)CN2CCC[C@H]2C)cc1NC(=O)c1cnn2cc(-c3cn(C)nc3C)sc12]

(R)-2-bromo-N-(2-methyl-5-(2-(2-methylpyrrolidin-1-yl)acetamido)pyridin-3-yl)pyrazolo[5,1-b]thiazole-7-carboxamide (103 mg, 0.207 mmol), 1,3-dimethyl-1H-pyrazole-4-boronic acid pinacol ester (119 mg, 0.535 mmol), cesium carbonate (209 mg, 0.642 mmol), 1,1′-bis(diphenylphosphino)ferrocene-palladium(II)dichloride dichloromethane complex (31.3 mg, 0.0383 mmol), and 1,4-dioxane:distilled water (5:1) (2.5 mL) were combined in a 2-5 mL microwave vial, equipped with a stir bar. The vial was sealed with a cap. The contents were sparged with nitrogen with vigorous stirring. The mixture was irradiated in a Biotage Initiator+microwave reactor at 130° C. for 1 h and allowed to cool to room temperature. Solvent was removed under reduced pressure. The crude residue was dissolved in MeOH and Si-Trisamine was added. The mixture was stirred at room temperature for approximately 20.5 h. The crude mixture was filtered using a Biotage phase separator. Solvent was removed under reduced pressure. The residue was purified by preparative high-performance liquid chromatography over column: Waters XBridge BEH C18 5 μm to give 2-(1,3-dimethyl-1H-pyrazol-4-yl)-N-(5-(2-(3-methoxypyrrolidin-1-yl)acetamido)-2-methylpyridin-3-yl)pyrazolo[5,1-b]thiazole-7-carboxamide as a light brown solid (21 mg, 20%). LCMS (ESI): mass calcd for C₂₄H₂₈N₈O₂S, 492.2; m/z found, 493.1 [M+H]+. ¹H NMR (DMSO-d6, 400 MHz) δ 9.8-9.9 (m, 2H), 8.60 (d, 1H, J=2.4 Hz), 8.52 (s, 1H), 8.39 (s, 1H), 8.17 (d, 1H, J=2.2 Hz), 8.08 (s, 1H), 3.80 (s, 3H), 3.46 (d, 1H, J=15.7 Hz), 3.1-3.2 (m, 1H), 3.03 (d, 1H, J=15.7 Hz), 2.5-2.6 (m, 1H), 2.40 (s, 3H), 2.3-2.4 (m, 4H), 1.9-2.0 (m, 1H), 1.6-1.8 (m, 2H), 1.3-1.5 (m, 1H), 1.07 (d, 3H, J=6.1 Hz).

### Example 441. 2-(1,3-Dimethyl-1H-pyrazol-4-yl)-N-(5-(2-(3-methoxyazetidin-1-yl)acetamido)-2-methylpyridin-3-yl)pyrazolo[5,1-b]thiazole-7-carboxamide

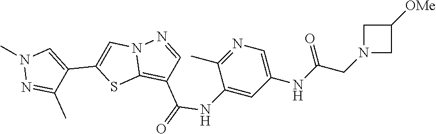 [SMILES: COC1CN(CC(=O)Nc2cnc(C)c(NC(=O)c3cnn4cc(-c5cn(C)nc5C)sc34)c2)C1]

2-Bromo-N-(5-(2-(3-methoxyazetidin-1-yl)acetamido)-2-methylpyridin-3-yl)pyrazolo[5,1-b]thiazole-7-carboxamide (71.5 mg, 0.149 mmol), 1,3-dimethyl-1H-pyrazole-4-boronic acid pinacol ester (67.2 mg, 0.303 mmol), cesium carbonate (149 mg, 0.454 mmol), 1,1′-bis(diphenylphosphino)ferrocene-palladium(II)dichloride dichloromethane complex (24.1 mg, 0.0295 mmol), and 1,4-dioxane:distilled water (5:1) (2.5 mL) were combined in a 2-5 mL microwave vial, equipped with a stir bar. The vial was sealed with a cap. The contents were sparged with nitrogen with vigorous stirring. The mixture was irradiated in a Biotage Initiator+microwave reactor at 130° C. for 1 h and allowed to cool to room temperature. Solvent was removed under reduced pressure in a 20 mL vial. The crude residue was dissolved in MeOH. Si-Trisamine, a metal scavenger, was added. The mixture was stirred at room temperature for approximately 15.5 h. The crude mixture was filtered using a Biotage phase separator. Solvent was removed under reduced pressure. The residue was purified by preparative high-performance liquid chromatography over column: Waters XBridge BEH C18 5 μm to afford 2-(1,3-dimethyl-1H-pyrazol-4-yl)-N-(5-(2-(3-methoxyazetidin-1-yl)acetamido)-2-methylpyridin-3-yl)pyrazolo[5,1-b]thiazole-7-carboxamide as a white solid (21 mg, 20%). LCMS (ESI): mass calcd for C₂₃H₂₆N₈O₃S, 494.2; m/z found, 495.1 [M+H]+. ¹H NMR (DMSO-d6, 400 MHz) δ 9.91 (s, 1H), 9.87 (s, 1H), 8.55 (d, 1H, J=2.2 Hz), 8.51 (s, 1H), 8.39 (s, 1H), 8.16 (d, 1H, J=2.0 Hz), 8.08 (s, 1H), 4.0-4.0 (m, 1H), 3.80 (s, 3H), 3.6-3.7 (m, 2H), 3.2-3.3 (m, 2H), 3.16 (s, 3H), 3.0-3.0 (m, 2H), 2.39 (s, 3H), 2.34 (s, 3H).

### Example 442. 2-(1,3-Dimethyl-1H-pyrazol-4-yl)-N-(5-(2-(3-methoxy-3-methylazetidin-1-yl)acetamido)-2-methylpyridin-3-yl)pyrazolo[5,1-b]thiazole-7-carboxamide

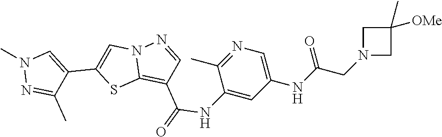 [SMILES: COC1(C)CN(CC(=O)Nc2cnc(C)c(NC(=O)c3cnn4cc(-c5cn(C)nc5C)sc34)c2)C1]

2-Bromo-N-(5-(2-(3-methoxy-3-methylazetidin-1-yl)acetamido)-2-methylpyridin-3-yl)pyrazolo[5,1-b]thiazole-7-carboxamide (38.2 mg, 0.0774 mmol), 1,3-dimethyl-1H-pyrazole-4-boronic acid pinacol ester (97.5 mg, 0.439 mmol), cesium carbonate (154 mg, 0.462 mmol), 1,1′-bis(diphenylphosphino)ferrocene-palladium(II)dichloride dichloromethane complex (14.4 mg, 0.0176 mmol), and 1,4-dioxane:distilled water (5:1) (2.8 mL) were combined in a 2-5 mL microwave vial, equipped with a stir bar. The vial was sealed with a cap. The contents were sparged with nitrogen with vigorous stirring. The mixture was irradiated in a Biotage Initiator+microwave reactor at 130° C. for 1 h and allowed to cool to room temperature. The crude residue was taken up in MeOH and passed through Si-Trisamine. Solvent was removed under reduced pressure.

The residue was purified by preparative high-performance liquid chromatography over column: Waters XBridge BEH C18 5 μm to provide 2-(1,3-dimethyl-1H-pyrazol-4-yl)-N-(5-(2-(3-methoxy-3-methylazetidin-1-yl)acetamido)-2-methylpyridin-3-yl)pyrazolo[5,1-b]thiazole-7-carboxamide as an off-white solid (18 mg, 42%). LCMS (ESI): mass calcd for C₂₄H₂₈N₈O₃S, 508.2; m/z found, 509.2 [M+H]+. ¹H NMR (DMSO-d6, 400 MHz) δ 9.89 (s, 2H), 8.55 (d, 1H, J=2.4 Hz), 8.52 (s, 1H), 8.39 (s, 1H), 8.14 (d, 1H, J=2.2 Hz), 8.08 (s, 1H), 3.80 (s, 3H), 3.2-3.3 (m, 4H), 3.14 (d, 2H, J=7.8 Hz), 3.11 (s, 3H), 2.40 (s, 3H), 2.34 (s, 3H), 1.42 (s, 3H).

### Example 443. (R)-2-(2,5-dimethyl-2H-1,2,3-triazol-4-yl)-N-(2-methyl-5-(2-(2-methylpyrrolidin-1-yl)acetamido)pyridin-3-yl)pyrazolo[5,1-b]thiazole-7-carboxamide

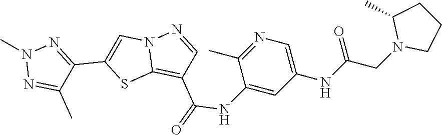 [SMILES: Cc1ncc(NC(=O)CN2CCC[C@H]2C)cc1NC(=O)c1cnn2cc(-c3nn(C)nc3C)sc12]

(R)-2-bromo-N-(2-methyl-5-(2-(2-methylpyrrolidin-1-yl)acetamido)pyridin-3-yl)pyrazolo[5,1-b]thiazole-7-carboxamide (103 mg, 0.208 mmol), 2,4-dimethyl-5-(4,4,5,5-tetramethyl-1,3,2-dioxaborolan-2-yl)-2H-1,2,3-triazole (110 mg, 0.468 mmol), cesium carbonate (205 mg, 0.618 mmol), 1,1′-bis(diphenylphosphino)ferrocene]dichloropalladium(II) (21.1 mg, 0.0288 mmol), and 1,4-dioxane:distilled water (5:1) (3.0 mL) were combined in a 2-5 mL microwave vial, equipped with a stir bar. The vial was sealed with a cap. The contents were sparged with nitrogen with vigorous stirring. The mixture was irradiated in a Biotage Initiator+microwave reactor at 130° C. for 1 h and allowed to cool to room temperature. Additional 2,4-dimethyl-5-(4,4,5,5-tetramethyl-1,3,2-dioxaborolan-2-yl)-2H-1,2,3-triazole (116 mg, 0.496 mmol), cesium carbonate (142 mg, 0.427 mmol), and 1,1′-bis(diphenylphosphino)ferrocene]dichloropalladium(II) (14.0 mg, 0.0191 mmol) were added to the vial. The mixture was irradiated in a Biotage Initiator+microwave reactor at 130° C. for 1 h and allowed to cool to room temperature. The crude residue was taken up in MeOH and passed through Si-Trisamine. Solvent was removed under reduced pressure. The residue was purified by preparative high-performance liquid chromatography over column: Kinetex 5 μm EVO C18 100 to give (R)-2-(2,5-dimethyl-2H-1,2,3-triazol-4-yl)-N-(2-methyl-5-(2-(2-methylpyrrolidin-1-yl)acetamido)pyridin-3-yl)pyrazolo[5,1-b]thiazole-7-carboxamide as an off-white solid (41 mg, 37%). LCMS (ESI): mass calcd for C₂₃H₂₇N₉O₂S, 493.2; m/z found, 494.2 [M+H]+. ¹H NMR (DMSO-d6, 400 MHz) δ 9.9-9.9 (m, 2H), 8.74 (s, 1H), 8.61 (d, 1H, J=2.4 Hz), 8.59 (s, 1H), 8.18 (d, 1H, J=2.2 Hz), 4.14 (s, 3H), 3.4-3.5 (m, 1H), 3.0-3.2 (m, 2H), 2.5-2.6 (m, 1H), 2.48 (s, 3H), 2.42 (s, 3H), 2.3-2.4 (m, 1H), 1.9-2.0 (m, 1H), 1.6-1.8 (m, 2H), 1.3-1.5 (m, 1H), 1.09 (d, 3H, J=6.1 Hz).

### Example 444. 2-(1,3-Dimethyl-1H-pyrazol-4-yl)-N-(5-(2-(2,2-dimethylpyrrolidin-1-yl)acetamido)-2-methylpyridin-3-yl)pyrazolo[5,1-b]thiazole-7-carboxamide

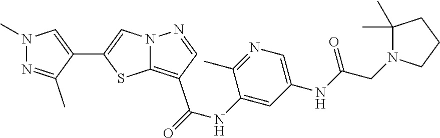 [SMILES: Cc1ncc(NC(=O)CN2CCCC2(C)C)cc1NC(=O)c1cnn2cc(-c3cn(C)nc3C)sc12]

2-Bromo-N-(5-(2-(2,2-dimethylpyrrolidin-1-yl)acetamido)-2-methylpyridin-3-yl)pyrazolo[5,1-b]thiazole-7-carboxamide (79.5 mg, 0.157 mmol), 1,3-dimethyl-1 h-pyrazole-4-boronic acid pinacol ester (109 mg, 0.489 mmol), cesium carbonate (185 mg, 0.557 mmol), 1,1′-bis(diphenylphosphino)ferrocene]dichloropalladium(II) (21.1 mg, 0.0288 mmol), and 1,4-dioxane:distilled water (5:1) (3.0 mL) were combined in a 2-5 mL microwave vial, equipped with a stir bar. The vial was sealed with a cap. The contents were sparged with nitrogen with vigorous stirring. The mixture was irradiated in a Biotage Initiator+microwave reactor at 130° C. for 1 h and allowed to cool to room temperature. Additional 1,3-dimethyl-1H-pyrazole-4-boronic acid pinacol ester (141 mg, 0.635 mmol), cesium carbonate (185 mg, 0.556 mmol), and 1,1′-bis(diphenylphosphino)ferrocene]dichloropalladium(II) (21.2 mg, 0.029 mmol) were added to the vial. The mixture was irradiated in a Biotage Initiator+microwave reactor at 130° C. for 1 h and allowed to cool to room temperature. The crude residue was taken up in MeOH and passed through Si-Trisamine. Solvent was removed under reduced pressure. The residue was purified by preparative high-performance liquid chromatography over column: Kinetex 5 μm EVO C18 100 to provide 2-(1,3-dimethyl-1H-pyrazol-4-yl)-N-(5-(2-(2,2-dimethylpyrrolidin-1-yl)acetamido)-2-methylpyridin-3-yl)pyrazolo[5,1-b]thiazole-7-carboxamide as a white solid (20 mg, 22%). LCMS (ESI): mass calcd for C₂₅H₃₀N₈O₂S, 506.2; m/z found, 507.2 [M+H]+.

### Example 445. 2-(2,5-Dimethyl-2H-1,2,3-triazol-4-yl)-N-(5-(2-(3,3-dimethylazetidin-1-yl)acetamido)-2-methylpyridin-3-yl)pyrazolo[5,1-b]thiazole-7-carboxamide

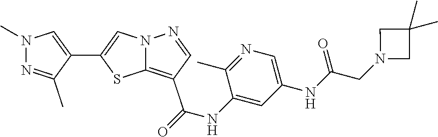

2-Bromo-N-(5-(2-(3,3-dimethylazetidin-1-yl)acetamido)-2-methylpyridin-3-yl)pyrazolo[5,1-b]thiazole-7-carboxamide (66.5 mg, 0.115 mmol), 2,4-dimethyl-5-(4,4,5,5-tetramethyl-1,3,2-dioxaborolan-2-yl)-2H-1,2,3-triazole (67.9 mg, 0.289 mmol), cesium carbonate (116 mg, 0.350 mmol), 1,1′-bis(diphenylphosphino)ferrocene-palladium(II)dichloride dichloromethane complex (13.1 mg, 0.0161 mmol), and 1,4-dioxane:distilled water (5:1) (2.5 mL) were combined in a 2-5 mL microwave vial, equipped with a stir bar. The vial was sealed with a cap. The contents were sparged with nitrogen with vigorous stirring. The mixture was irradiated in a Biotage Initiator+microwave reactor at 130° C. for 1 h and allowed to cool to room temperature. The crude residue was taken up in MeOH and passed through Si-Trisamine. Solvent was removed under reduced pressure. The residue was purified by preparative high-performance liquid chromatography over column: Kinetex 5 μm EVO C18 100 to supply 2-(2,5-dimethyl-2H-1,2,3-triazol-4-yl)-N-(5-(2-(3,3-dimethylazetidin-1-yl)acetamido)-2-methylpyridin-3-yl)pyrazolo[5,1-b]thiazole-7-carboxamide as a white solid (16 mg, 25%). LCMS (ESI): mass calcd for C₂₃H₂₇N₉O₂S, 493.2; m/z found, 494.2 [M+H]+. ¹H NMR (DMSO-d6, 400 MHz) δ 9.92 (s, 1H), 9.84 (s, 1H), 8.74 (s, 1H), 8.58 (s, 1H), 8.57 (d, 1H, J=2.4 Hz), 8.15 (d, 1H, J=2.4 Hz), 4.14 (s, 3H), 3.23 (s, 2H), 3.05 (s, 4H), 2.48 (s, 3H), 2.41 (s, 3H), 1.21 (s, 6H).

### Example 446. 2-(1,3-Dimethyl-1H-pyrazol-4-yl)-N-(2-methyl-5-(2-(3-methylpyrrolidin-1-yl)acetamido)pyridin-3-yl)pyrazolo[5,1-b]thiazole-7-carboxamide

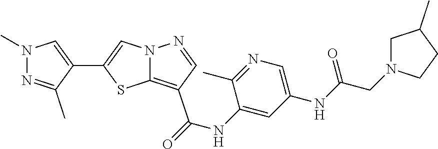 [SMILES: Cc1ncc(NC(=O)CN2CCC(C)C2)cc1NC(=O)c1cnn2cc(-c3cn(C)nc3C)sc12]

### Step a: 2-Bromo-N-(2-methyl-5-(2-(3-methylpyrrolidin-1-yl)acetamido)pyridin-3-yl)pyrazolo[5,1-b]thiazole-7-carboxamide

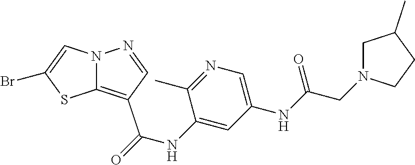 [SMILES: Cc1ncc(NC(=O)CN2CCC(C)C2)cc1NC(=O)c1cnn2cc(Br)sc12]

A 30 mL vial, equipped with a stir bar, was charged with 2-bromo-N-(5-(2-chloroacetamido)-2-methylpyridin-3-yl)pyrazolo[5,1-b]thiazole-7-carboxamide (304 mg, 0.644 mmol), K₂CO₃ (304 mg, 2.20 mmol), DMF (3.2 mL, 41 mmol), and 3-methylpyrrolidine (189 mg, 2.18 mmol). A nitrogen atmosphere was established. The mixture was heated to 50° C. After approximately 26 h, the mixture was allowed to cool to room temperature, poured into water (100 mL), and allowed to stir for 20 min. Precipitate was collected via filtration. The filter cake was washed with water. The solid was allowed to air-dry under vacuum overnight to provide 2-bromo-N-(2-methyl-5-(2-(3-methylpyrrolidin-1-yl)acetamido)pyridin-3-yl)pyrazolo[5,1-b]thiazole-7-carboxamide as an off-white solid (110 mg, 34%). LCMS (ESI): mass calcd for C₁₉H₂₁BrN₆O₂S, 476.1; m/z found, 477.0 [M+H]+. ¹H NMR (DMSO-d6, 400 MHz) δ 9.95 (s, 1H), 9.91 (s, 1H), 8.78 (s, 1H), 8.60 (s, 1H), 8.58 (d, 1H, J=2.4 Hz), 8.16 (d, 1H, J=2.2 Hz), 3.2-3.3 (m, 2H), 2.8-2.9 (m, 1H), 2.7-2.8 (m, 1H), 2.58 (dt, 1H, J=5.9, 8.7 Hz), 2.39 (s, 3H), 2.2-2.3 (m, 1H), 2.1-2.2 (m, 1H), 1.9-2.0 (m, 1H), 1.32 (tdd, 1H, J=6.1, 8.5, 12.3 Hz), 1.00 (d, 3H, J=6.8 Hz).

### Step b: 2-(1,3-Dimethyl-1H-pyrazol-4-yl)-N-(2-methyl-5-(2-(3-methylpyrrolidin-1-yl)acetamido)pyridin-3-yl)pyrazolo[5,1-b]thiazole-7-carboxamide

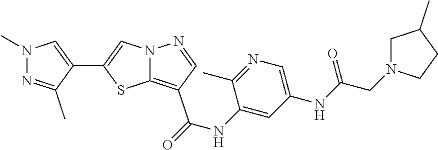 [SMILES: Cc1ncc(NC(=O)CN2CCC(C)C2)cc1NC(=O)c1cnn2cc(-c3cn(C)nc3C)sc12]

2-Bromo-N-(2-methyl-5-(2-(3-methylpyrrolidin-1-yl)acetamido)pyridin-3-yl)pyrazolo[5,1-b]thiazole-7-carboxamide (56.1 mg, 0.112 mmol), 1,3-dimethyl-1H-pyrazole-4-boronic acid pinacol ester (62.1 mg, 0.28 mmol), cesium carbonate (112 mg, 0.337 mmol), 1,1′-bis(diphenylphosphino)ferrocene-palladium(II)dichloride dichloromethane complex (13.4 mg, 0.0164 mmol), and 1,4-dioxane:distilled water (5:1) (2.5 mL) were combined in a 2-5 mL microwave vial, equipped with a stir bar. The vial was sealed with a cap. The contents were sparged with nitrogen with vigorous stirring. The mixture was irradiated in a Biotage Initiator+microwave reactor at 130° C. for 1 h and allowed to cool to room temperature. Additional 1,3-dimethyl-1H-pyrazole-4-boronic acid pinacol ester (63.0 mg, 0.284 mmol) was added. The mixture was heated again. More 1,3-dimethyl-1H-pyrazole-4-boronic acid pinacol ester (109 mg, 0.492), cesium carbonate (139 mg, 0.425 mmol), and 1,1′-bis(diphenylphosphino)ferrocene-palladium(II)dichloride dichloromethane complex (6.8 mg, 8.33 μmol) were added to the reaction vial. Nitrogen was blown over the vial headspace, and the vial was sealed with a cap. The mixture was irradiated in a Biotage Initiator+microwave reactor at 130° C. for 1 h and allowed to cool to room temperature. Solvent was removed under reduced pressure. The crude residue was taken up in MeOH and passed through Si-Trisamine. Solvent was removed under reduced pressure. The residue was purified by preparative high-performance liquid chromatography over column: Kinetex 5 μm EVO C8 150 Å, 100×30 mm to afford 2-(1,3-dimethyl-1H-pyrazol-4-yl)-N-(2-methyl-5-(2-(3-methylpyrrolidin-1-yl)acetamido)pyridin-3-yl)pyrazolo[5,1-b]thiazole-7-carboxamide as a light yellow solid (38 mg, 56%). LCMS (ESI): mass calcd for C₂₄H₂₈N₈O₂S, 492.2; m/z found, 493.2 [M+H]+. ¹H NMR (DMSO-d6, 400 MHz) δ 10.79 (s, 1H), 9.90 (s, 1H), 8.5-8.6 (m, 2H), 8.41 (s, 1H), 8.2-8.2 (m, 1H), 8.08 (s, 1H), 4.3-4.3 (m, 2H), 3.6-3.7 (m, 4H), 3.1-3.4 (m, 3H), 2.45 (s, 3H), 2.34 (s, 4H), 2.1-2.2 (m, 1H), 1.5-1.7 (m, 1H), 1.07 (dd, 3H, J=1.7, 6.8 Hz).

### Example 447: N-(5-(2-(2,2-dimethylpyrrolidin-1-yl)acetamido)-2-methylpyridin-3-yl)-2-(1-(2-hydroxyethyl)-1H-pyrazol-4-yl)pyrazolo[5,1-b]thiazole-7-carboxamide

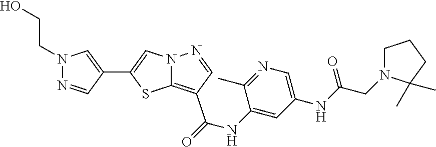 [SMILES: Cc1ncc(NC(=O)CN2CCCC2(C)C)cc1NC(=O)c1cnn2cc(-c3cnn(CCO)c3)sc12]

### Step a: tert-butyl (5-(2-(1-(2-hydroxyethyl)-1H-pyrazol-4-yl)pyrazolo[5,1-b]thiazole-7-carboxamido)-6-methylpyridin-3-yl)carbamate

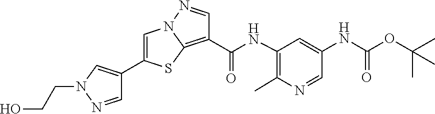 [SMILES: Cc1ncc(NC(=O)OC(C)(C)C)cc1NC(=O)c1cnn2cc(-c3cnn(CCO)c3)sc12]

To a mixture of tert-butyl (5-(6-bromo-[1,2,3]triazolo[1,5-a]pyridine-3-carboxamido)-6-methylpyridin-3-yl)carbamate (792 mg, 1.75 mmol) and 1-(2-hydroxyethyl-1H-pyrazole-4-boronic acid pinacol ester (500.26 mg, 2.10 mmol) in 1,4-dioxane (10 mL) was added a solution of K₂CO₃ (725.97 mg, 5.26 mmol) in water (2 mL) followed by the addition of 1,1′-bis(diphenylphosphino)ferrocene-palladium(II)dichloride dichlormethane complex (285.98 mg, 0.35 mmol). The reaction mixture was thoroughly flushed with argon before being capped and heated at 100° C. for 20.5 h. The reaction was diluted with MeOH (25 mL) and silica gel (10 g) was added. All solvents were removed in vacuo. The silica gel mesh was loaded on a Redi Sep Rf silica gel cartridge (40 g) eluting with CH₂Cl₂ over 5 min then MeOH/CH₂Cl₂ (0-30%) over 15 min to afford a brown solid. The solid was dissolved in 20% MeOH/CH₂Cl₂ (25 mL) and silica gel (5 g) was added. All solvents were removed in vacuo. The silica gel mesh was loaded on a Redi Sep Rf silica gel cartridge (40 g) eluting with EtOAc over 5 min then MeOH/EtOAc (0-30%) over 15 min to afford the product, tert-butyl (5-(2-(1-(2-hydroxyethyl)-1H-pyrazol-4-yl)pyrazolo[5,1-b]thiazole-7-carboxamido)-6-methylpyridin-3-yl)carbamate, as a tan solid (237.9 mg). LCMS (ESI): mass calcd. for C₂₂H₂₅N₇O₄S, 483.2; m/z found, 484.2 [M+H]⁺. ¹H NMR (400 MHz, DMSO-d6) δ ppm 1.48 (s, 9H), 2.36 (s, 3H), 3.74-3.81 (m, 2H), 4.19 (t, J=1.00 Hz, 2H), 4.96 (t, J=1.00 Hz, 1H), 7.90 (s, 1H), 7.98 (br s, 1H), 8.20 (s, 1H), 8.37 (d, J=1.00 Hz, 1H), 8.49 (s, 1H), 8.60 (s, 1H), 9.55 (br s, 1H), 9.84 (s, 1H).

### Step b: N-(5-amino-2-methylpyridin-3-yl)-2-(1-(2-hydroxyethyl)-1H-pyrazol-4-yl)pyrazolo[5,1-b]thiazole-7-carboxamide

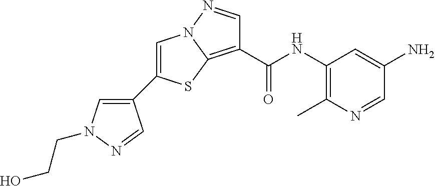 [SMILES: Cc1ncc(N)cc1NC(=O)c1cnn2cc(-c3cnn(CCO)c3)sc12]

To a mixture of tert-butyl (5-(2-(1-(2-hydroxyethyl)-1H-pyrazol-4-yl)pyrazolo[5,1-b]thiazole-7-carboxamido)-6-methylpyridin-3-yl)carbamate (237 mg, 0.49 mmol) in DCM (10 mL) was added HCl (4M in dioxane) (3.06 mL, 4 M, 12.25 mmol). The reaction was stirred at 25° C. for 22 h. All solvents were removed in vacuo. The residue was dried under high vacuum to afford the HCl salt of the product, N-(5-amino-2-methylpyridin-3-yl)-2-(1-(2-hydroxyethyl)-1H-pyrazol-4-yl)pyrazolo[5,1-b]thiazole-7-carboxamide, as a light yellow solid (quantitative yield). The product was used without further purification. LCMS (ESI): mass calcd. for C₁₇H₁₇N₇O₂S, 383.1; m/z found, 384.1 [M+H]⁺. ¹H NMR (400 MHz, DMSO-d6) δ ppm 2.53 (s, 3H), 3.77 (t, J=1.00 Hz, 2H), 4.19 (t, J=1.00 Hz, 2H), 6.04 (br s, 4H), 7.76 (d, J=1.00 Hz, 1H), 7.83-7.86 (m, 1H), 7.91 (s, 1H), 8.22 (s, 1H), 8.61 (s, 1H), 8.67 (s, 1H), 10.35 (s, 1H).

### Step c: 2-(4-(7-((5-(2-chloroacetamido)-2-methylpyridin-3-yl)carbamoyl)pyrazolo[5,1-b]thiazol-2-yl)-1H-pyrazol-1-yl)ethyl 2-chloroacetate

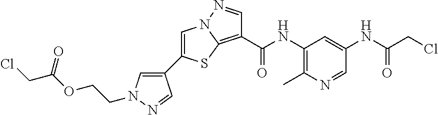 [SMILES: Cc1ncc(NC(=O)CCl)cc1NC(=O)c1cnn2cc(-c3cnn(CCOC(=O)CCl)c3)sc12]

To a suspension of N-(5-amino-2-methylpyridin-3-yl)-2-(1-(2-hydroxyethyl)-1H-pyrazol-4-yl)pyrazolo[5,1-b]thiazole-7-carboxamide hydrochloride (205 mg, 0.49 mmol) and Et₃N (0.20 mL, 0.728 g/mL, 1.47 mmol) in DCM (6 mL) was added chloroacetyl chloride (0.047 mL, 1.418 g/mL, 0.59 mmol). The reaction was stirred at 25° C. for 5 days. The reaction was diluted with CH₂Cl₂ (25 mL) and silica gel (5 g) was added and all solvents were removed in vacuo. The silica gel mesh was loaded on a Redi Sep Rf silica gel cartridge (40 g) eluting with EtOAc over 5 min then MeOH/EtOAc (0-30%) over 15 min to afford the product, 2-(4-(7-((5-(2-chloroacetamido)-2-methylpyridin-3-yl)carbamoyl)pyrazolo[5,1-b]thiazol-2-yl)-1H-pyrazol-1-yl)ethyl 2-chloroacetate, as a tan solid (210.1 mg). LCMS (ESI): mass calcd. for C₂₁H₁₉C₁₂N₇O₄S, 535.1; m/z found, 536.0 [M+H]⁺. ¹H NMR (400 MHz, DMSO-d6) δ ppm 2.42 (s, 3H), 3.17 (s, 2H), 4.25 (s, 1H), 4.31 (s, 2H), 4.39 (s, 3H), 7.94 (s, 1H), 8.14 (d, J=1.00 Hz, 1H), 8.26 (s, 1H), 8.53 (s, 2H), 8.60 (s, 1H), 9.92 (s, 1H), 10.58 (s, 1H).

### Step d: N-(5-(2-(2,2-dimethylpyrrolidin-1-yl)acetamido)-2-methylpyridin-3-yl)-2-(1-(2-hydroxyethyl)-1H-pyrazol-4-yl)pyrazolo[5,1-b]thiazole-7-carboxamide

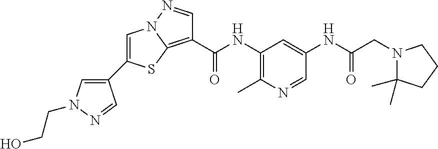 [SMILES: Cc1ncc(NC(=O)CN2CCCC2(C)C)cc1NC(=O)c1cnn2cc(-c3cnn(CCO)c3)sc12]

To a solution of 2-(4-(7-((5-(2-chloroacetamido)-2-methylpyridin-3-yl)carbamoyl)pyrazolo[5,1-b]thiazol-2-yl)-1H-pyrazol-1-yl)ethyl 2-chloroacetate (200 mg, 0.37 mmol) and 2,2-dimethylpyrrolidine hydrochloride (81.35 mg, 0.60 mmol) in DMF (1.5 mL) was added K₂CO₃ (309.19 mg, 2.24 mmol). The reaction was heated at 50° C. for 4 days. LCMS indicated a mixture of the hydrolysis of the ester product and the bis-acylated product about 1:1. Water (3 drops) was added and heating was continued at 50° C. for 8 days. The reaction was filtered through an Acrodisc CR 13 mm syringe filter with a 0.45 μm PTFE membrane. The reaction mixture was purified by HPLC (H₂O/ACN, 0.16% NH₄OH, XBridge 19×100) to afford the product, N-(5-(2-(2,2-dimethylpyrrolidin-1-yl)acetamido)-2-methylpyridin-3-yl)-2-(1-(2-hydroxyethyl)-1H-pyrazol-4-yl)pyrazolo[5,1-b]thiazole-7-carboxamide, as a tan solid (80.5 mg). LCMS (ESI): mass calcd. for C₂₅H₃₀N₈O₃S, 522.2; m/z found, 523.2 [M+H]⁺. ¹H NMR (400 MHz, DMSO-d6) δ ppm 1.02 (s, 6H), 1.60-1.71 (m, 2H), 1.77 (quin, J=1.00 Hz, 2H), 2.41 (s, 3H), 2.78 (t, J=1.00 Hz, 2H), 3.15 (s, 2H), 3.77 (q, J=1.00 Hz, 2H), 4.18 (t, J=1.00 Hz, 2H), 4.95 (t, J=1.00 Hz, 1H), 7.90 (s, 1H), 8.15 (d, J=1.00 Hz, 1H), 8.21 (s, 1H), 8.50 (s, 1H), 8.58 (s, 1H), 8.65 (d, J=1.00 Hz, 1H), 9.73 (s, 1H), 9.88 (s, 1H).

### Example 448. N-(5-(2-(2,2-dimethylpyrrolidin-1-yl)acetamido)-2-methylpyridin-3-yl)-2-(2-(2-fluoroethoxy)pyridin-3-yl)pyrazolo[5,1-b]thiazole-7-carboxamide

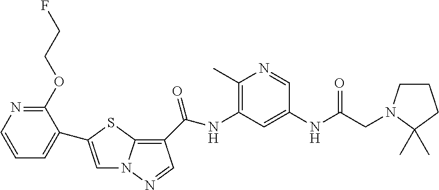 [SMILES: Cc1ncc(NC(=O)CN2CCCC2(C)C)cc1NC(=O)c1cnn2cc(-c3cccnc3OCCF)sc12]

### Step a: ethyl 2-(2-hydroxypyridin-3-yl)pyrazolo[5,1-b]thiazole-7-carboxylate

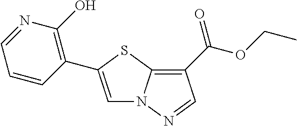 [SMILES: CCOC(=O)c1cnn2cc(-c3cccnc3O)sc12]

A mixture of ethyl 2-(2-methoxypyridin-3-yl)pyrazolo[5,1-b]thiazole-7-carboxylate (210 mg, 0.692 mmol) in 4N HCl in dioxane (4 mL, 4 M, 16 mmol) was heated at 90° C. for 2.5 h. The reaction mixture was cooled to rt and diluted with diethyl ether. The mixture was filtered and the solid was dried to give ethyl 2-(2-hydroxypyridin-3-yl)pyrazolo[5,1-b]thiazole-7-carboxylate hydrochloride (210 mg) which will be used in next step without further purification. LCMS (ESI): mass calcd. for C₁₃H₁₁N₃O₃S, 289.05; m/z found, 290.0 [M+H]⁺.

### Step b: ethyl 2-(2-(2-fluoroethoxy)pyridin-3-yl)pyrazolo[5,1-b]thiazole-7-carboxylate

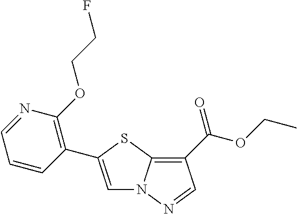 [SMILES: CCOC(=O)c1cnn2cc(-c3cccnc3OCCF)sc12]

To a mixture of give ethyl 2-(2-hydroxypyridin-3-yl)pyrazolo[5,1-b]thiazole-7-carboxylate hydrochloride (50 mg, 0.153 mmol), 2-fluoroethan-1-ol (20 mg, 0.312 mmol), and PPh₃ (120 mg, 0.458 mmol) in THE (4 mL) was added DIAD (0.15 mL, 0.771 mmol). The reaction was stirred at rt for 7 h. Additional 2-fluoroethan-1-ol (10 mg, 0.156 mmol), PPh₃ (40 mg, 0.153 mmol), and DIAD (0.05 mL, 0.257 mmol) was added. The reaction was stirred at rt overnight. The reaction mixture was concentrated and the residue was purified with silica gel column (30-60% EtOAc/heptane) to give ethyl 2-(2-(2-fluoroethoxy)pyridin-3-yl)pyrazolo[5,1-b]thiazole-7-carboxylate, LCMS (ESI): mass calcd. for C₁₅H₁₄FN₃O₃S, 335.07; m/z found, 336.1 [M+H]⁺, followed by ethyl 2-(1-(2-fluoroethyl)-2-oxo-1,2-dihydropyridin-3-yl)pyrazolo[5,1-b]thiazole-7-carboxylate, LCMS (ESI): mass calcd. for C₁₅H₁₄FN₃O₃S, 335.07; m/z found, 336.1 [M+H]⁺.

### Step c: 2-(2-(2-fluoroethoxy)pyridin-3-yl)pyrazolo[5,1-b]thiazole-7-carboxylic acid

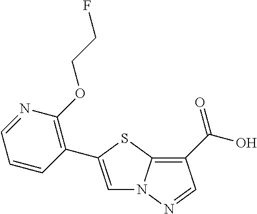 [SMILES: O=C(O)c1cnn2cc(-c3cccnc3OCCF)sc12]

A mixture of ethyl 2-(2-(2-fluoroethoxy)pyridin-3-yl)pyrazolo[5,1-b]thiazole-7-carboxylate (25.2 mg, 0.075 mmol) and LiOH (15 mg, 0.626 mmol) in THE (2 mL) and H₂O (1 mL) was stirred at rt for 4 days. Additional LiOH (15 mg, 0.626 mmol) was added. The reaction was stirred at rt for one more day. The reaction mixture was diluted with H₂O (2 mL) and extracted with CH₂Cl₂. The aqueous layer was collected and the pH was adjusted to ˜3 with 2N HCl. The resulting mixture was extracted with 10% MeOH/CH₂Cl₂ (3×). The organic layer was dried over Na₂SO₄ and concentrated to give 2-(2-(2-fluoroethoxy)pyridin-3-yl)pyrazolo[5,1-b]thiazole-7-carboxylic acid (16 mg) which will be used in next step without further purification.

### Step d: N-(5-(2-(2,2-dimethylpyrrolidin-1-yl)acetamido)-2-methylpyridin-3-yl)-2-(2-(2-fluoroethoxy)pyridin-3-yl)pyrazolo[5,1-b]thiazole-7-carboxamide

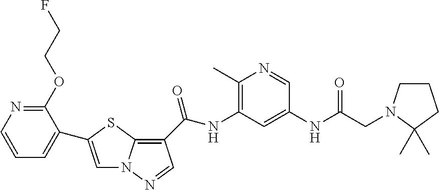 [SMILES: Cc1ncc(NC(=O)CN2CCCC2(C)C)cc1NC(=O)c1cnn2cc(-c3cccnc3OCCF)sc12]

A solution of 2-(2-(2-fluoroethoxy)pyridin-3-yl)pyrazolo[5,1-b]thiazole-7-carboxylic acid (16 mg, 0.0521 mmol), N-(5-amino-6-methylpyridin-3-yl)-2-(2,2-dimethylpyrrolidin-1-yl)acetamide (15 mg, 0.0572 mmol), and EDCI (15 mg, 0.0782 mmol) in pyridine (1.5 mL) was heated at 70° C. for 7 h. The reaction mixture was concentrated and the residue was purified with silica gel column (4-6% MeOH/CH₂Cl₂ with 0.2-0.3% NH₄OH) followed by reverse phase HPLC (30-80% ACN/H₂O with 10 mM NH₄OH, 8 min) to give N-(5-(2-(2,2-dimethylpyrrolidin-1-yl)acetamido)-2-methylpyridin-3-yl)-2-(2-(2-fluoroethoxy)pyridin-3-yl)pyrazolo[5,1-b]thiazole-7-carboxamide (7 mg, yield 24%). LCMS (ESI): mass calcd. for C₂₇H₃₀FN₇O₃S, 551.21; m/z found, 552.3 [M+H]⁺. ¹H NMR (CHLOROFORM-d) δ: 9.27 (br s, 1H), 8.64 (d, J=2.4 Hz, 1H), 8.50 (s, 1H), 8.41-8.47 (m, 1H), 8.19-8.25 (m, 1H), 8.15 (dd, J=4.9, 2.0 Hz, 1H), 7.73-7.86 (m, 1H), 7.68 (s, 1H), 7.05 (dd, J=7.8, 4.9 Hz, 1H), 4.86-4.97 (m, 1H), 4.69-4.86 (m, 3H), 3.20 (s, 2H), 2.87 (t, J=7.3 Hz, 2H), 2.55 (s, 3H), 1.73-1.91 (m, 4H), 1.07 (s, 6H).

### Example 449. N-(5-(2-(2,2-dimethylpyrrolidin-1-yl)acetamido)-2-methylpyridin-3-yl)-2-(1-(2-fluoroethyl)-2-oxo-1,2-dihydropyridin-3-yl)pyrazolo[5,1-b]thiazole-7-carboxamide

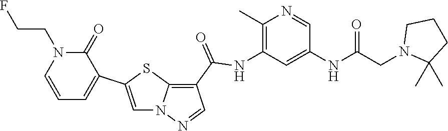 [SMILES: Cc1ncc(NC(=O)CN2CCCC2(C)C)cc1NC(=O)c1cnn2cc(-c3cccn(CCF)c3=O)sc12]

### Step a: ethyl 2-(1-(2-fluoroethyl)-2-oxo-1,2-dihydropyridin-3-yl)pyrazolo[5,1-b]thiazole-7-carboxylate

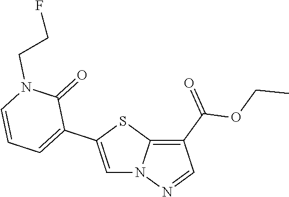 [SMILES: CCOC(=O)c1cnn2cc(-c3cccn(CCF)c3=O)sc12]

The synthesis of ethyl 2-(1-(2-fluoroethyl)-2-oxo-1,2-dihydropyridin-3-yl)pyrazolo[5,1-b]thiazole-7-carboxylate is described in Step b of Example 448.

### Step b: 2-(1-(2-fluoroethyl)-2-oxo-1,2-dihydropyridin-3-yl)pyrazolo[5,1-b]thiazole-7-carboxylic acid

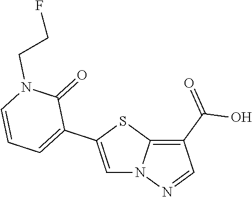 [SMILES: O=C(O)c1cnn2cc(-c3cccn(CCF)c3=O)sc12]

A mixture of ethyl 2-(1-(2-fluoroethyl)-2-oxo-1,2-dihydropyridin-3-yl)pyrazolo[5,1-b]thiazole-7-carboxylate (20 mg, 0.0596 mmol) and LiOH (12 mg, 0.501 mmol) in H₂O (1 mL) and THF (2 mL) was stirred at rt for 4 days. The aqueous layer was collected and the pH was adjusted to ˜3 with 2N HCl. The resulting mixture was extracted with 10% MeOH/CH₂Cl₂ (4×). The organic layer was dried over Na₂SO₄ and concentrated to give 2-(1-(2-fluoroethyl)-2-oxo-1,2-dihydropyridin-3-yl)pyrazolo[5,1-b]thiazole-7-carboxylic acid (13 mg, yield 71%) which will be used in next step without further purification.

### Step c: N-(5-(2-(2,2-dimethylpyrrolidin-1-yl)acetamido)-2-methylpyridin-3-yl)-2-(1-(2-fluoroethyl)-2-oxo-1,2-dihydropyridin-3-yl)pyrazolo[5,1-b]thiazole-7-carboxamide

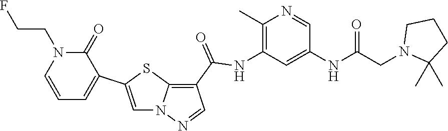 [SMILES: Cc1ncc(NC(=O)CN2CCCC2(C)C)cc1NC(=O)c1cnn2cc(-c3cccn(CCF)c3=O)sc12]

A solution of 2-(1-(2-fluoroethyl)-2-oxo-1,2-dihydropyridin-3-yl)pyrazolo[5,1-b]thiazole-7-carboxylic acid (13 mg, 0.0423 mmol), N-(5-amino-6-methylpyridin-3-yl)-2-(2,2-dimethylpyrrolidin-1-yl)acetamide (12 mg, 0.0457 mmol), and EDCI (12 mg, 0.0626 mmol) in pyridine (1.5 mL) was heated at 70° C. for 7 h. The reaction mixture was concentrated and the residue was purified with silica gel column (6% MeOH/CH₂Cl₂ with 0.3% NH₄OH) to give N-(5-(2-(2,2-dimethylpyrrolidin-1-yl)acetamido)-2-methylpyridin-3-yl)-2-(1-(2-fluoroethyl)-2-oxo-1,2-dihydropyridin-3-yl)pyrazolo[5,1-b]thiazole-7-carboxamide (6.5 mg, yield 28%). LCMS (ESI): mass calcd. for C₂₇H₃₀FN₇O₃S, 551.21; m/z found, 552.2 [M+H]⁺. ¹H NMR (CHLOROFORM-d) δ: 9.26 (br s, 1H), 8.85 (s, 1H), 8.69 (d, J=2.4 Hz, 1H), 8.42 (d, J=2.0 Hz, 1H), 8.17 (s, 1H), 7.61-7.78 (m, 1H), 7.56 (s, 1H), 7.43 (d, J=6.4 Hz, 1H), 6.38 (t, J=7.1 Hz, 1H), 4.80-4.92 (m, 1H), 4.68-4.80 (m, 1H), 4.29-4.46 (m, 2H), 3.19 (s, 2H), 2.76-2.96 (m, 2H), 2.56 (s, 3H), 1.75-1.92 (m, 4H), 1.01-1.12 (m, 6H).

### Example 450, 2-(6,7-dihydro-4H-pyrazolo[5,1-c][1,4]oxazin-3-yl)-N-(5-((2-(2,2-dimethylpyrrolidin-1-yl)ethyl)carbamoyl)-2-methylpyridin-3-yl)pyrazolo[5,1-b]thiazole-7-carboxamide

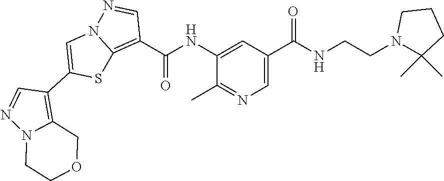 [SMILES: Cc1ncc(C(=O)NCCN2CCCC2(C)C)cc1NC(=O)c1cnn2cc(-c3cnn4c3COCC4)sc12]

### Step a: 1-(2-((tert-butyldimethylsilyl)oxy)ethyl)-4-iodo-1H-pyrazole

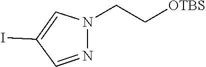 [SMILES: CC(C)(C)[Si](C)(C)OCCn1cc(I)cn1]

To a solution of 4-iodo-1H-pyrazole (5.0 g, 26 mmol) and (2-bromoethoxy)(tert-butyl)dimethyl-silane (6.7 g, 28 mmol) in MeCN (80 ml) was added CS₂CO₃ (13 g, 40 mmol) at 0° C. The reaction mixture was stirred at room temperature for 16 hrs. Then the reaction mixture was filtered. The filter cake was washed with MTBE (40 ml). The combined filtrate was concentrated under vacuum to give crude as colorless oil, which was purified by column chromatography over silica gel (eluent: petroleum ether:ethyl acetate from 100:0 to 80:20). The desired fractions were collected and the solvent was concentrated under vacuum to give desired product 1-(2-((tert-butyldimethylsilyl)oxy)ethyl)-4-iodo-1H-pyrazole (9 g, 99%) as a white solid. LCMS (ESI): mass calcd. for C₁₁H₂₁₁IN₂OSi, 352.0; m/z found, 353.1 [M+H]⁺. ¹HNMR (400 MHz, CDCl₃) δ: 7.56 (d, J=4.3 Hz, 2H), 4.28 (t, J=5.2 Hz, 2H), 3.96 (t, J=5.2 Hz, 2H), 0.91 (s, 9H), 0.00 (s, 6H).

### Step b: 1-(2-((tert-butyldimethylsilyl)oxy)ethyl)-4-iodo-1H-pyrazole-5-carbaldehyde

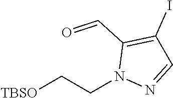 [SMILES: CC(C)(C)[Si](C)(C)OCCn1ncc(I)c1C=O]

To a solution of 1-(2-((tert-butyldimethylsilyl)oxy)ethyl)-4-iodo-1H-pyrazole (3.7 g, 11 mmol) in THE (100 mL) was added LDA (8.1 mL, 16 mmol) dropwise at −78° C. under N₂ atmosphere. The reaction mixture was stirred at −78° C. for 0.5 hr. Then DMF (1.6 mL, 21 mmol) was added dropwise. The reaction mixture was stirred at −78° C. for 1 hr. The reaction mixture was quenched with saturated NH₄Cl at 0° C. Then the mixture was extracted with TBME (150 mL*3). The combined organic layer was dried over Na₂SO₄, filtered, and the solvent was evaporated to give crude product as pale brown oil, which was purified by column chromatography over silica gel (eluent: petroleum ether/ethyl acetate=100:0 to 96:4). The fractions were collected and the solvent was removed to give target 1-(2-((tert-butyldimethylsilyl)oxy)ethyl)-4-iodo-1H-pyrazole-5-carbaldehyde (2.3 g, 58%) as a pale yellow solid. ¹H NMR (400 MHz, CDCl₃) δ: 9.88 (s, 1H), 7.68 (s, 1H), 4.76 (t, J=5.7 Hz, 2H), 3.99 (t, J=5.7 Hz, 2H), 0.91-0.83 (m, 9H), 0.04-−0.06 (m, 6H).

### Step c: 3-iodo-6,7-dihydro-4H-pyrazolo[5,1-c][1,4]oxazine

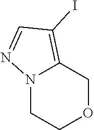

To a solution of 1-(2-((tert-butyldimethylsilyl)oxy)ethyl)-4-iodo-1H-pyrazole-5-carbaldehyde (6.9 g, 18 mmol) in CH₂Cl₂ (135 mL) was added Et₃SiH (8.8 mL, 55 mmol) and TFA (8.2 mL, 111 mmol) dropwise at 0° C. The reaction mixture was stirred at room temperature for 16 hrs. The reaction mixture was adjusted to pH-8 with saturated NaHCO₃. Then the mixture was extracted with DCM (150 mL*3). The combined organic layer was dried over Na₂SO₄, filtered, and the solvent was evaporated to give crude product as pale brown oil, which was purified by column chromatography over silica gel (eluent: petroleum ether/ethyl acetate=100:0 to 93:7). The desired fractions were collected and the solvent was concentrated under vacuum to give desired product 3-iodo-6,7-dihydro-4H-pyrazolo[5,1-c][1,4]oxazine (2.6 g, 58%) as a yellow solid. LCMS (ESI): mass calcd. for C₆H₇IN₂O, 250.0; m/z found, 251.0 [M+H]⁺. ¹H NMR (400 MHz, CDCl₃) δ: 7.53 (s, 1H), 4.74 (s, 2H), 4.25-4.18 (m, 2H), 4.14-4.08 (m, 2H).

### Step d: 3-(4,4,5,5-tetramethyl-1,3,2-dioxaborolan-2-yl)-6,7-dihydro-4H-pyrazolo[5,1-c][1,4]oxazine

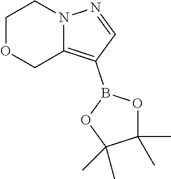 [SMILES: CC1(C)OB(c2cnn3c2COCC3)OC1(C)C]

To a mixture of 3-iodo-6,7-dihydro-4H-pyrazolo[5,1-c][1,4]oxazine (1.6 g, 6.4 mmol), 4,4,4′,4′,5,5,5′,5′-octamethyl-2,2′-bi(1,3,2-dioxaborolane) (2.5 g, 9.8 mmol) and KOAc (2.1 g, 21 mmol) in dioxane (80 mL) was added Xphos-Pd-G₄ (441 mg, 0.51 mmol) at room temperature under N₂ atmosphere. The reaction mixture was purged with N₂ for 2 minutes. Then the reaction mixture was stirred at 95° C. for 12 hrs. The reaction mixture was concentrated in vacuo to give black solid, which was purified by column chromatography over silica gel (eluent: petroleum ether/ethyl acetate=100:0 to 80:20). The desired fractions were collected and the solvent was concentrated under vacuum to give desired product 3-(4,4,5,5-tetramethyl-1,3,2-dioxaborolan-2-yl)-6,7-dihydro-4H-pyrazolo[5,1-c][1,4]oxazine (0.8 g, 50%) as a pale yellow oil. LCMS (ESI): mass calcd. for C₁₂H₁₉BN₂O₃, 250.1; m/z found, 251.2 [M+H]⁺.

### Step e: 5-(2-(6,7-dihydro-4H-pyrazolo[5,1-c][1,4]oxazin-3-yl)pyrazolo[5,1-b]thiazole-7-carboxamido)-6-methylnicotinic acid

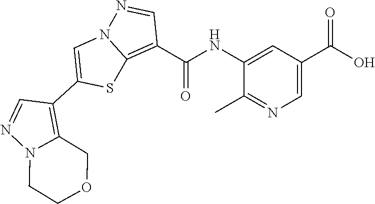 [SMILES: Cc1ncc(C(=O)O)cc1NC(=O)c1cnn2cc(-c3cnn4c3COCC4)sc12]

To a mixture of methyl 5-(2-bromopyrazolo[5,1-b]thiazole-7-carboxamido)-6-methylnicotinate (210 mg, 0.53 mmol), 3-(4,4,5,5-tetramethyl-1,3,2-dioxaborolan-2-yl)-6,7-dihydro-4H-pyrazolo[5,1-c][1,4]oxazine (280 mg, 0.85 mmol) and K₂CO₃ (226 mg, 1.6 mmol) in dioxane (8 mL) and H₂O (2 mL) was added Pd(dppf)Cl₂·CH₂Cl₂ (130 mg, 0.16 mmol) at room temperature under N₂ atmosphere. The reaction mixture was purged with N₂ for 2 minutes. The reaction mixture was stirred at 90° C. for 16 hrs. The reaction mixture was concentrated in vacuo to give black solid. The black solid was dissolved in H₂O (20 mL) then adjusted to pH-5 with 1N HCl. Then the mixture was filtered. The filter cake was washed with H₂O (30 mL). The filter cake was dried under vacuum to give black solid, which was triturated with DCM/CH₃OH (20:1, 63 mL). The mixture was filtered. The filter cake was dried under vacuum to give target 5-(2-(6,7-dihydro-4H-pyrazolo[5,1-c][1,4]oxazin-3-yl)pyrazolo[5,1-b]thiazole-7-carboxamido)-6-methylnicotinic acid (202 mg, 59%) as a brown solid. LCMS (ESI): mass calcd. for C₁₉H₁₆N₆O₄S, 424.1; m/z found, 425.2 [M+H]⁺.

### Step f: 2-(6,7-dihydro-4H-pyrazolo[5,1-c][1,4]oxazin-3-yl)-N-(5-((2-(2,2-dimethylpyrrolidin-1-yl)ethyl)carbamoyl)-2-methylpyridin-3-yl)pyrazolo[5,1-b]thiazole-7-carboxamide

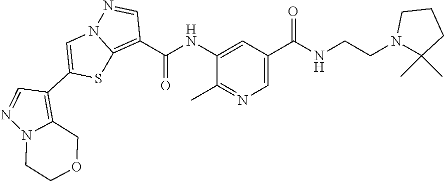 [SMILES: Cc1ncc(C(=O)NCCN2CCCC2(C)C)cc1NC(=O)c1cnn2cc(-c3cnn4c3COCC4)sc12]

To a solution of 5-(2-(6,7-dihydro-4H-pyrazolo[5,1-c][1,4]oxazin-3-yl)pyrazolo[5,1-b]thiazole-7-carboxamido)-6-methylnicotinic acid (125 mg, 0.16 mmol), HATU (95 mg, 0.25 mmol) and N,N-diisopropylethylamine (0.11 mL, 0.63 mmol) in DMF (6 mL) was added 2-(2,2-dimethylpyrrolidin-1-yl)ethanamine (55 mg, 0.39 mmol). The resulting mixture was stirred at room temperature for 2 hrs and then concentrated under vacuum to give the crude product, which was purified by preparative high-performance liquid chromatography over Column: Phenomenex C18 80*40 mm*3 um to give the title compound 2-(6,7-dihydro-4H-pyrazolo[5,1-c][1,4]oxazin-3-yl)-N-(5-((2-(2,2-dimethylpyrrolidin-1-yl)ethyl)carbamoyl)-2-methylpyridin-3-yl)pyrazolo[5,1-b]thiazole-7-carboxamide (16.1 mg, 18%) as a white solid. LCMS (ESI): mass calcd. for C₂₇H₃₂N₈O₃S, 548.2; m/z found, 549.2 [M+H]⁺. ¹H NMR (400 MHz, METHANOL-d₄) δ: 8.83 (s, 1H), 8.45 (s, 1H), 8.38 (d, J=1.5 Hz, 1H), 8.12 (s, 1H), 7.84 (s, 1H), 5.08 (s, 2H), 4.24 (br d, J=4.9 Hz, 2H), 4.22-4.17 (m, 2H), 3.75 (br t, J=6.1 Hz, 2H), 3.50 (br s, 2H), 3.23 (br s, 2H), 2.64 (s, 3H), 2.10 (br d, J=7.4 Hz, 2H), 2.02 (br d, J=7.3 Hz, 2H), 1.36 (br s, 6H).

### Example 451. (S)—N-(5-(2-(1-isopropylpyrrolidin-2-yl)acetamido)-2-methylpyridin-3-yl)-2-(1-(2-methoxyethyl)-1H-pyrazol-4-yl)pyrazolo[5,1-b]thiazole-7-carboxamide

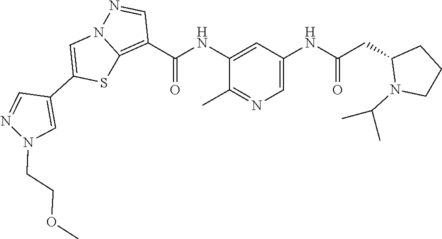 [SMILES: COCCn1cc(-c2cn3ncc(C(=O)Nc4cc(NC(=O)C[C@@H]5CCCN5C(C)C)cnc4C)c3s2)cn1]

### Step a: (S)-methyl 2-(1-isopropylpyrrolidin-2-yl)acetate

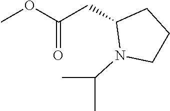

To a solution of (S)-methyl 2-(pyrrolidin-2-yl)acetate hydrochloride (500 mg, 1.8 mmol), propan-2-one (1 mL, 18 mmol) and TEA (1.5 mL, 11 mmol) in DCE (10 mL), then NaBH₃CN (200 mg, 3.2 mmol) was added and stirred at room temperature. The reaction was stirred at 25° C. for 2 hrs. The mixture was concentrated under vacuum to afford product as brown oil. The brown oil was purified by silica gel column chromatography (eluent: DCM:MeOH from 100:0 to 90:10). The fractions were collected and the solvent was removed to give (S)-methyl 2-(1-isopropylpyrrolidin-2-yl)acetate (330 mg, 98%) as yellow solid. ¹H NMR (400 MHz, CHLOROFORM-d) δ ppm: 3.50-3.61 (m, 3H), 3.37-3.49 (m, 1H), 3.06-3.28 (m, 2H), 2.65-2.74 (m, 2H), 2.49 (br dd, J=16.69, 8.58 Hz, 1H), 1.81-2.09 (m, 2H), 1.56-1.80 (m, 2H), 1.15 (d, J=6.56 Hz, 3H), 1.05 (d, J=6.68 Hz, 3H).

### Step b: (S)-2-(1-isopropylpyrrolidin-2-yl)acetic acid

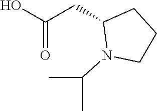

To a solution of (S)-methyl 2-(1-isopropylpyrrolidin-2-yl)acetate (300 mg, 1.6 mmol) in THF/MeOH=1/1 (6 mL) was added lithium hydroxide hydrate (70 mg, 1.7 mmol) in H₂O (2 mL) and the reaction was stirred at 20° C. for 1 hour. TLC (DCM/MeOH=10/1, Rf=0.4, UV) showed a new spot was observed and the starting material was consumed. The mixture was concentrated to dryness under vacuum to give (S)-2-(1-isopropylpyrrolidin-2-yl)acetic acid (270 mg, 97%) as a brown solid.

### Step c: (S)—N-(5-(2-(1-isopropylpyrrolidin-2-yl)acetamido)-2-methylpyridin-3-yl)-2-(1-(2-methoxyethyl)-1H-pyrazol-4-yl)pyrazolo[5,1-b]thiazole-7-carboxamide

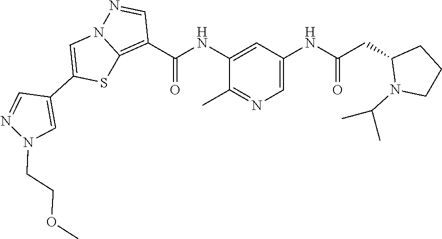 [SMILES: COCCn1cc(-c2cn3ncc(C(=O)Nc4cc(NC(=O)C[C@@H]5CCCN5C(C)C)cnc4C)c3s2)cn1]

To a mixture of N-(5-amino-2-methylpyridin-3-yl)-2-(1-(2-methoxyethyl)-1H-pyrazol-4-yl)pyrazolo[5,1-b]thiazole-7-carboxamide (200 mg, 0.41 mmol) in DMF (5 mL) was added (S)-2-(1-isopropylpyrrolidin-2-yl)acetic acid (90 mg, 0.53 mmol) and DIEA (280 μL, 1.7 mmol), then HATU (190 mg, 0.50 mmol) was added. The mixture was stirred at 25° C. for 2 hours and then concentrated under vacuum to give the crude product, which was purified by preparative high-performance liquid chromatography over column: Phenomenex C18 80*40 mm*3 um and by Supercritical Fluid Chromatography over column: DAICEL CHIRALPAK AD (250 mm*30 mm, 10 um) to give the title compound (S)—N-(5-(2-(1-isopropylpyrrolidin-2-yl)acetamido)-2-methylpyridin-3-yl)-2-(1-(2-methoxyethyl)-1H-pyrazol-4-yl)pyrazolo[5,1-b]thiazole-7-carboxamide (32 mg, 26%) as a white solid. LCMS (ESI): mass calcd. for C₂₇H₃₄N₈O₃S, 550.7; m/z found, 551.3 [M+H]⁺. ¹H NMR (400 MHz, METHANOL-d₄) δ: 8.56 (d, J=2.26 Hz, 1H), 8.41 (s, 1H), 8.26 (s, 1H), 8.23 (d, J=2.27 Hz, 1H), 8.09 (s, 1H), 7.87 (s, 1H), 4.37 (t, J=5.19 Hz, 2H), 3.79 (t, J=5.19 Hz, 2H), 3.36 (s, 3H), 2.90-3.19 (m, 2H), 2.58-2.74 (m, 2H), 2.51 (s, 3H), 2.40-2.50 (m, 1H), 1.94-2.11 (m, 1H), 1.74-1.89 (m, 2H), 1.66 (br d, J=6.32 Hz, 1H), 1.25-1.47 (m, 1H), 1.22 (br d, J=6.56 Hz, 3H), 1.03-1.13 (m, 3H).

### Example 452. (S)-2-(1-(2-methoxyethyl)-1H-pyrazol-4-yl)-N-(2-methyl-5-(1-((2-methylpyrrolidin-1-yl)methyl)cyclopropanecarboxamido)pyridin-3-yl)pyrazolo[5,1-b]thiazole-7-carboxamide

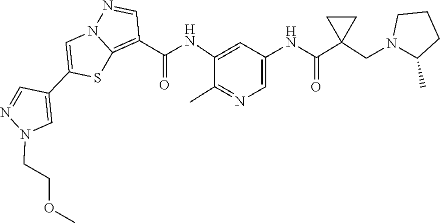 [SMILES: COCCn1cc(-c2cn3ncc(C(=O)Nc4cc(NC(=O)C5(CN6CCC[C@@H]6C)CC5)cnc4C)c3s2)cn1]

### Step a: (S)-methyl 1-((2-methylpyrrolidin-1-yl)methyl)cyclopropanecarboxylate

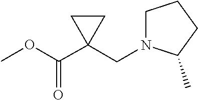

A mixture of methyl 1-formylcyclopropanecarboxylate (500 mg, 3.9 mmol), (S)-2-methylpyrrolidine (1 g, 11 mmol) and acetic acid (950 μL, 11 mmol) in DCE (5 mL), then Na(CN)BH₃ (500 mg, 7.9 mmol) was added and stirred at room temperature for 2 hours. The as colourless oil was purified by flash column chromatography over a 12 g silica gel (gradient: DCM:MeOH from 100:0 to 90:10). The filtrate was then concentrated to dryness under reduced pressure to afford the crude product (S)-methyl 1-((2-methylpyrrolidin-1-yl)methyl)cyclopropanecarboxylate (290 mg, 37%) as a white oil. LCMS (ESI): mass calcd. for C₁₁H₁₉NO₂, 197.2; m/z found, 198.2 [M+H]⁺. ¹H NMR (400 MHz, CHLOROFORM-d) δ: 3.68 (br s, 1H), 3.63 (s, 3H), 3.46 (br d, J=13.7 Hz, 1H), 3.28 (br s, 1H), 3.09-2.94 (m, 2H), 2.22-2.02 (m, 2H), 2.02-1.89 (m, 1H), 1.87-1.72 (m, 1H), 1.50-1.34 (m, 5H), 1.16-1.06 (m, 2H).

### Step b: (S)-1-((2-methylpyrrolidin-1-yl)methyl)cyclopropanecarboxylic acid

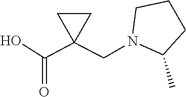

To a solution of (S)-methyl 1-((2-methylpyrrolidin-1-yl)methyl)cyclopropanecarboxylate (240 mg, 1.2 mmol) in mixture of MeOH:THF:H₂O=1:1:1 (4.5 mL) was added LiOH (102 mg, 2.4 mmol). The mixture was stirred at room temperature for 2 h. and then concentrated under vacuum to give pure product (S)-1-((2-methylpyrrolidin-1-yl)methyl)cyclopropanecarboxylic acid (200 mg, 89%) as a white solid. LCMS (ESI): mass calcd. for C₁₀H₁₇NO₂, 183.2; m/z found, 184.2 [M+H]⁺.

### Step c: (S)-2-(1-(2-methoxyethyl)-1H-pyrazol-4-yl)-N-(2-methyl-5-(1-((2-methylpyrrolidin-1-yl)methyl)cyclopropanecarboxamido)pyridin-3-yl)pyrazolo[5,1-b]thiazole-7-carboxamide

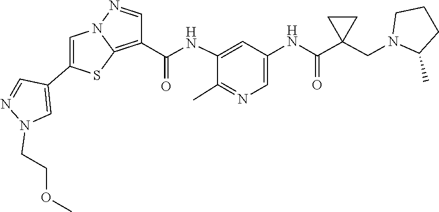 [SMILES: COCCn1cc(-c2cn3ncc(C(=O)Nc4cc(NC(=O)C5(CN6CCC[C@@H]6C)CC5)cnc4C)c3s2)cn1]

To the mixture of N-(5-amino-2-methylpyridin-3-yl)-2-(1-(2-methoxyethyl)-1H-pyrazol-4-yl)pyrazolo[5,1-b]thiazole-7-carboxamide (120 mg, 0.27 mmol) in DMF (5 mL) was added (S)-1-((2-methylpyrrolidin-1-yl)methyl)cyclopropanecarboxylic acid (108 mg, 0.59 mmol) and N-(chloro(dimethylamino)methylene)-N-methylmethanaminium hexafluorophosphate(V) (180 mg, 0.64 mmol), then 1-methyl-1H-imidazole (180 mg, 2.2 mmol) was added. The resulting mixture was stirred at room temperature for 2 hours, and then concentrated under vacuum to give the crude product, which was purified by preparative high-performance liquid chromatography over column: Phenomenex C18 75*30 mm*3 um to give the title compound (S)-2-(1-(2-methoxyethyl)-1H-pyrazol-4-yl)-N-(2-methyl-5-(1-((2-methylpyrrolidin-1-yl)methyl)cyclopropane-carboxamido)pyridin-3-yl)pyrazolo[5,1-b]thiazole-7-carboxamide (30 mg, 19%) as a white solid. LCMS (ESI): mass calcd. for C₂₈H₃₄N₈O₃S, 562.6; m/z found, 563.2 [M+H]⁺. ¹H NMR (400 MHz, CHLOROFORM-d) δ: 12.35 (s, 1H), 8.67 (d, J=2.3 Hz, 1H), 8.27 (d, J=2.1 Hz, 1H), 8.11 (s, 1H), 7.80 (s, 1H), 7.71 (d, J=4.6 Hz, 2H), 7.58 (s, 1H), 4.35 (t, J=5.1 Hz, 2H), 3.80 (t, J=5.1 Hz, 2H), 3.64 (d, J=13.1 Hz, 1H), 3.46-3.35 (m, 4H), 2.58-2.46 (m, 4H), 2.23 (q, J=8.8 Hz, 1H), 2.13-2.02 (m, 1H), 1.94-1.77 (m, 2H), 1.72 (d, J=12.9 Hz, 1H), 1.60-1.49 (m, 2H), 1.32-1.23 (m, 4H), 0.81-0.70 (m, 1H), 0.57-0.48 (m, 1H).

### Example 453. N-(5-(2-((cyclopropylmethyl)amino)acetamido)-2-methylpyridin-3-yl)-2-(6,7-dihydro-5H-pyrazolo[5,1-b][1,3]oxazin-3-yl)pyrazolo[5,1-b]thiazole-7-carboxamide

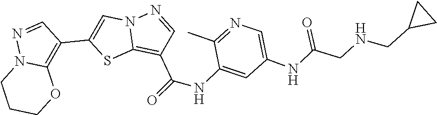 [SMILES: Cc1ncc(NC(=O)CNCC2CC2)cc1NC(=O)c1cnn2cc(-c3cnn4c3OCCC4)sc12]

### Step a: 2-bromo-N-(5-(2-((cyclopropylmethyl)amino)acetamido)-2-methylpyridin-3-yl)pyrazolo[5,1-b]thiazole-7-carboxamide

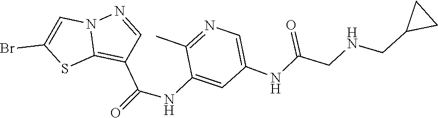 [SMILES: Cc1ncc(NC(=O)CNCC2CC2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with cyclopropylmethylamine (88 mg, 50%). LCMS (ESI): mass calcd. for C₁₈H₁₉BrN₆O₂S, 463.4; m/z found, 463.0/465.0 [M+H]⁺.

### Step b: N-(5-(2-(5-oxa-2-azaspiro[3.5]nonan-2-yl)acetamido)-2-methylpyridin-3-yl)-2-(6,7-dihydro-5H-pyrazolo[5,1-b][1,3]oxazin-3-yl)pyrazolo[5,1-b]thiazole-7-carboxamide

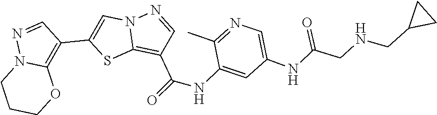 [SMILES: Cc1ncc(NC(=O)CNCC2CC2)cc1NC(=O)c1cnn2cc(-c3cnn4c3OCCC4)sc12]

The title compound was prepared from N-(5-(2-(5-oxa-2-azaspiro[3.5]nonan-2-yl)acetamido)-2-methylpyridin-3-yl)-2-bromopyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (13 mg, 22%). LCMS (ESI): mass calcd. for C₂₄H₂₆N₈O₃S, 506.6; m/z found, 507.3 [M+H]⁺. ¹H NMR (METHANOL-d4) δ: 8.70 (d, J=2.0 Hz, 1H), 8.36-8.41 (m, 2H), 8.09 (s, 1H), 7.68 (s, 1H), 4.49-4.55 (m, 2H), 4.20 (t, J=6.1 Hz, 2H), 4.07 (s, 2H), 3.02 (d, J=7.8 Hz, 2H), 2.58 (s, 3H), 2.30-2.39 (m, 2H), 1.07-1.21 (m, 1H), 0.72-0.80 (m, 2H), 0.41-0.48 (m, 2H).

### Example 454. N-(5-(2-((cyclopropylmethyl)amino)acetamido)-2-methylpyridin-3-yl)-2-(5,6-dihydro-4H-pyrrolo[1,2-b]pyrazol-3-yl)pyrazolo[5,1-b]thiazole-7-carboxamide

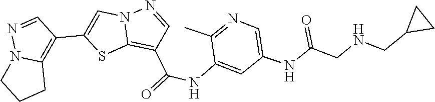 [SMILES: Cc1ncc(NC(=O)CNCC2CC2)cc1NC(=O)c1cnn2cc(-c3cnn4c3CCC4)sc12]

The title compound was prepared from N-(5-(2-(5-oxa-2-azaspiro[3.5]nonan-2-yl)acetamido)-2-methylpyridin-3-yl)-2-bromopyrazolo[5,1-b]thiazole-7-carboxamide according to the procedure of Example 372, substituting 3-(tetramethyl-1,3,2-dioxaborolan-2-yl)-4H,5H,6H-pyrrolo[1,2-b]pyrazole for 3-fluoro-5-(4,4,5,5-tetramethyl-1,3,2-dioxaborolan-2-yl)pyridin-2-amine (18 mg, 31%). LCMS (ESI): mass calcd. for C₂₄H₂₆N₈O₂S, 490.6; m/z found, 491.2 [M+H]⁺. ¹H NMR (METHANOL-d4) δ: 8.71 (d, J=2.4 Hz, 1H), 8.39-8.43 (m, 2H), 8.17 (s, 1H), 7.83 (s, 1H), 4.19 (t, J=7.3 Hz, 2H), 4.07 (s, 2H), 3.09-3.15 (m, 2H), 3.02 (d, J=7.3 Hz, 2H), 2.70-2.80 (m, 2H), 2.58 (s, 3H), 1.08-1.18 (m, 1H), 0.72-0.80 (m, 2H), 0.41-0.48 (m, 2H).

### Example 455. 2-(6,7-dihydro-5H-pyrazolo[5,1-b][1,3]oxazin-3-yl)-N-(2-methyl-5-(2-(propylamino)acetamido)pyridin-3-yl)pyrazolo[5,1-b]thiazole-7-carboxamide

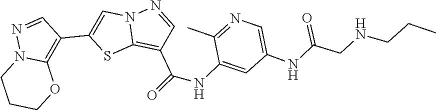 [SMILES: CCCNCC(=O)Nc1cnc(C)c(NC(=O)c2cnn3cc(-c4cnn5c4OCCC5)sc23)c1]

### Step a: 2-bromo-N-(2-methyl-5-(2-(propylamino)acetamido)pyridin-3-yl)pyrazolo[5,1-b]thiazole-7-carboxamide

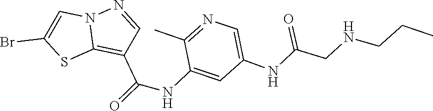 [SMILES: CCCNCC(=O)Nc1cnc(C)c(NC(=O)c2cnn3cc(Br)sc23)c1]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with propylamine (77 mg, 46%). LCMS (ESI): mass calcd. for C₁₇H₁₉BrN₆O₂S, 451.3; m/z found, 451.0/453.0 [M+H]⁺.

### Step b: 2-(6,7-dihydro-5H-pyrazolo[5,1-b][1,3]oxazin-3-yl)-N-(2-methyl-5-(2-(propylamino)acetamido)pyridin-3-yl)pyrazolo[5,1-b]thiazole-7-carboxamide

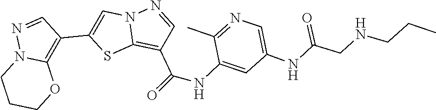 [SMILES: CCCNCC(=O)Nc1cnc(C)c(NC(=O)c2cnn3cc(-c4cnn5c4OCCC5)sc23)c1]

The title compound was prepared from N-(5-(2-(5-oxa-2-azaspiro[3.5]nonan-2-yl)acetamido)-2-methylpyridin-3-yl)-2-bromopyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (7.5 mg, 18%). LCMS (ESI): mass calcd. for C₂₃H₂₆N₈O₃S, 494.6; m/z found, 495.2 [M+H]⁺. ¹H NMR (METHANOL-d4) δ: 8.58 (d, J=2.4 Hz, 1H), 8.34 (s, 1H), 8.24 (d, J=2.4 Hz, 1H), 8.04 (s, 1H), 7.66 (s, 1H), 4.48-4.55 (m, 2H), 4.18 (t, J=6.1 Hz, 2H), 3.42 (s, 2H), 2.60 (t, J=7.3 Hz, 2H), 2.49 (s, 3H), 2.29-2.38 (m, 2H), 1.56 (sxt, J=7.3 Hz, 2H), 0.96 (t, J=7.3 Hz, 3H).

### Example 456. 2-(5,6-dihydro-4H-pyrrolo[1,2-b]pyrazol-3-yl)-N-(2-methyl-5-(2-(propylamino)acetamido)pyridin-3-yl)pyrazolo[5,1-b]thiazole-7-carboxamide

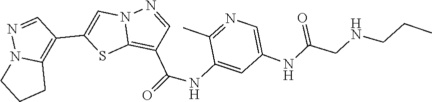 [SMILES: CCCNCC(=O)Nc1cnc(C)c(NC(=O)c2cnn3cc(-c4cnn5c4CCC5)sc23)c1]

The title compound was prepared from N-(5-(2-(5-oxa-2-azaspiro[3.5]nonan-2-yl)acetamido)-2-methylpyridin-3-yl)-2-bromopyrazolo[5,1-b]thiazole-7-carboxamide according to the procedure of Example 372, substituting 3-(tetramethyl-1,3,2-dioxaborolan-2-yl)-4H,5H,6H-pyrrolo[1,2-b]pyrazole for 3-fluoro-5-(4,4,5,5-tetramethyl-1,3,2-dioxaborolan-2-yl)pyridin-2-amine (4.5 mg, 11%). LCMS (ESI): mass calcd. for C₂₃H₂₆N₈O₂S, 478.6; m/z found, 479.2 [M+H]⁺. ¹H NMR (METHANOL-d4) δ: 8.58 (d, J=2.4 Hz, 1H), 8.37 (s, 1H), 8.24 (d, J=2.4 Hz, 1H), 8.14 (s, 1H), 7.83 (s, 1H), 4.18 (t, J=7.3 Hz, 2H), 3.43 (s, 2H), 3.07-3.15 (m, 2H), 2.74 (quin, J=7.3 Hz, 2H), 2.58-2.64 (m, 2H), 2.49 (s, 3H), 1.57 (sxt, J=7.4 Hz, 2H), 0.97 (t, J=7.3 Hz, 3H).

### Example 457: N-(5-(2-(2,2-dimethylpyrrolidin-1-yl)acetamido)-2-methylpyridin-3-yl)-2-(1-(1-hydroxy-2-methylpropan-2-yl)-1H-pyrazol-4-yl)pyrazolo[5,1-b]thiazole-7-carboxamide

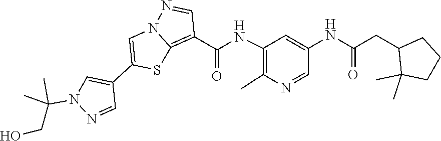 [SMILES: Cc1ncc(NC(=O)CC2CCCC2(C)C)cc1NC(=O)c1cnn2cc(-c3cnn(C(C)(C)CO)c3)sc12]

### Step a: tert-butyl (5-(2-(1-(1-hydroxy-2-methylpropan-2-yl)-1H-pyrazol-4-yl)pyrazolo[5,1-b]thiazole-7-carboxamido)-6-methylpyridin-3-yl)carbamate

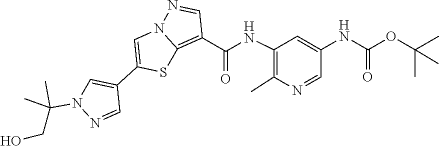 [SMILES: Cc1ncc(NC(=O)OC(C)(C)C)cc1NC(=O)c1cnn2cc(-c3cnn(C(C)(C)CO)c3)sc12]

To a mixture of tert-butyl (5-(6-bromo-[1,2,3]triazolo[1,5-a]pyridine-3-carboxamido)-6-methylpyridin-3-yl)carbamate (709 mg, 1.57 mmol) and 2-methyl-2-(4-(4,4,5,5-tetramethyl-1,3,2-dioxaborolan-2-yl)-1H-pyrazol-1-yl)propan-1-ol (500.60 mg, 1.88 mmol) in 1,4-dioxane (10 mL) was added a solution of K₂CO₃ (649.89 mg, 4.70 mmol) in water (2 mL) followed by the addition of 1,1′-bis(diphenylphosphino)ferrocene-palladium(II)dichloride dichlormethane complex (256.01 mg, 0.31 mmol). The reaction was thoroughly flushed with argon before being capped and heated at 100° C. for 18 h. The reaction was removed from the heat. The reaction was diluted with MeOH (25 mL) and silica gel (10 g) was added. All solvents were removed in vacuo. The silica gel mesh was loaded on a Redi Sep Rf silica gel cartridge (40 g) eluting with CH₂Cl₂ over 5 min then MeOH/CH₂Cl₂ (0-30%) over 15 min to afford a brown solid. The solid was dissolved in 20% MeOH/CH₂Cl₂ (25 mL) and silica gel (5 g) was added. All solvents were removed in vacuo. The silica gel mesh was loaded on a Redi Sep Rf silica gel cartridge (40 g) eluting with EtOAc over 5 min then MeOH/EtOAc (0-30%) over 15 min to afford the product, tert-butyl (5-(2-(1-(1-hydroxy-2-methylpropan-2-yl)-1H-pyrazol-4-yl)pyrazolo[5,1-b]thiazole-7-carboxamido)-6-methylpyridin-3-yl)carbamate, as a tan solid (307.4 mg). LCMS (ESI): mass calcd. for C₂₄H₂₉N₇O₄S, 511.2; m/z found, 512.2 [M+H]⁺. ¹H NMR (400 MHz, DMSO-d6) δ ppm 1.48 (s, 9H), 1.51 (s, 6H), 2.37 (s, 3H), 3.61 (d, J=1.00 Hz, 2H), 5.02 (t, J=1.00 Hz, 1 H), 7.89 (s, 1H), 7.99 (br s, 1H), 8.28 (s, 1H), 8.38 (d, J=1.00 Hz, 1H), 8.49 (s, 1H), 8.58 (s, 1H), 9.56 (br s, 1H), 9.85 (s, 1H).

### Step b: N-(5-amino-2-methylpyridin-3-yl)-2-(1-(1-hydroxy-2-methylpropan-2-yl)-1H-pyrazol-4-yl)pyrazolo[5,1-b]thiazole-7-carboxamide hydrochloride

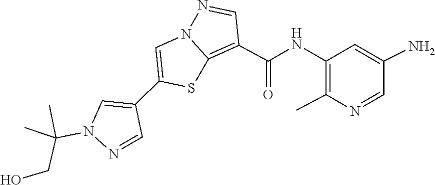 [SMILES: Cc1ncc(N)cc1NC(=O)c1cnn2cc(-c3cnn(C(C)(C)CO)c3)sc12]

To a suspension of tert-butyl (5-(2-(1-(1-hydroxy-2-methylpropan-2-yl)-1H-pyrazol-4-yl)pyrazolo[5,1-b]thiazole-7-carboxamido)-6-methylpyridin-3-yl)carbamate (307 mg, 0.6 mmol) in DCM (10 mL) was added HCl (4M in dioxane) (3.75 mL, 4 M, 15.00 mmol). The reaction was stirred at 25° C. for 22 h. All solvents were removed in vacuo. The residue was dried under high vacuum to afford the HCl salt of the product, N-(5-amino-2-methylpyridin-3-yl)-2-(1-(1-hydroxy-2-methylpropan-2-yl)-1H-pyrazol-4-yl)pyrazolo[5,1-b]thiazole-7-carboxamide, as a light yellow solid (quantitative yield). The product was used without further purification. LCMS (ESI): mass calcd. for C₁₉H₂₁N₇O₂S, 411.1; m/z found, 412.1 [M+H]⁺. ¹H NMR (400 MHz, DMSO-d6) δ ppm 1.50 (s, 6H), 2.54 (s, 3H), 3.57 (s, 2H), 6.10 (br s, 3H), 7.76 (d, J=1.00 Hz, 1H), 7.83 (d, J=1.00 Hz, 1H), 7.90 (s, 1H), 8.29 (s, 1H), 8.59 (s, 1H), 8.68 (s, 1H), 10.37 (s, 1H).

### Step c: 2-(4-(7-((5-(2-chloroacetamido)-2-methylpyridin-3-yl)carbamoyl)pyrazolo[5,1-b]thiazol-2-yl)-1H-pyrazol-1-yl)-2-methylpropyl 2-chloroacetate

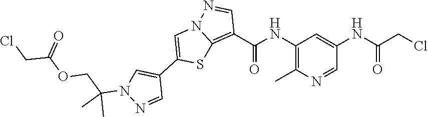 [SMILES: Cc1ncc(NC(=O)CCl)cc1NC(=O)c1cnn2cc(-c3cnn(C(C)(C)COC(=O)CCl)c3)sc12]

To a mixture of N-(5-amino-2-methylpyridin-3-yl)-2-(1-(1-hydroxy-2-methylpropan-2-yl)-1H-pyrazol-4-yl)pyrazolo[5,1-b]thiazole-7-carboxamide hydrochloride (268 mg, 0.60 mmol) in DCM (10 mL) was added Et₃N (0.33 mL, 0.728 g/mL, 2.39 mmol) followed by the addition of chloroacetyl chloride (0.095 mL, 1.42 g/mL, 1.20 mmol). The reaction was stirred at 25° C. under argon for 4 days. LCMS indicated a ratio of about 4:2:1 starting amino pyridine, mono-acylation, and bis-acylation products. The reaction was diluted with CH₂Cl₂ (5 mL), Et₃N (0.5 mL) was added, followed by the addition of chloroacetyl chloride (0.5 mL). The reaction was stirred at 25° C. under argon for 10 days. Silica gel (5 g) was added to the reaction and all solvents were removed in vacuo. The silica gel mesh was loaded on a Redi Sep Rf silica gel cartridge (40 g) eluting with EtOAc over 5 min then MeOH/EtOAc (0-30%) over 15 min to afford a mixture of products. The products were taken up in 20% MeOH/CH₂Cl₂ (25 mL) and silica gel (5 g). All solvents were removed in vacuo and the silica gel mesh was loaded on a Redi Sep Rf silica gel cartridge (40 g) eluting with CH₂Cl₂ over 5 min then MeOH/CH₂Cl₂ (0-30%) over 15 min to afford the product, 2-(4-(7-((5-(2-chloroacetamido)-2-methylpyridin-3-yl)carbamoyl)pyrazolo[5,1-b]thiazol-2-yl)-1H-pyrazol-1-yl)-2-methylpropyl 2-chloroacetate, as a tan solid (121.6 mg). LCMS (ESI): mass calcd. for C₂₃H₂₃C₁₂N₇O₄S, 563.1; m/z found, 564.1 [M+H]⁺. ¹H NMR (400 MHz, DMSO-d6) δ ppm 1.60 (s, 6H), 2.42 (s, 3H), 4.29 (s, 2H), 4.38 (s, 2H), 4.43 (s, 2H), 7.95 (s, 1H), 8.14 (d, J=1.00 Hz, 1H), 8.37 (s, 1H), 8.51 (s, 1H), 8.54 (d, J=1.00 Hz, 1H), 8.59 (s, 1H), 9.88 (s, 1H), 10.54 (s, 1H).

### Step d: N-(5-(2-(2,2-dimethylpyrrolidin-1-yl)acetamido)-2-methylpyridin-3-yl)-2-(1-(1-hydroxy-2-methylpropan-2-yl)-1H-pyrazol-4-yl)pyrazolo[5,1-b]thiazole-7-carboxamide

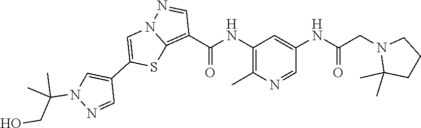 [SMILES: Cc1ncc(NC(=O)CN2CCCC2(C)C)cc1NC(=O)c1cnn2cc(-c3cnn(C(C)(C)CO)c3)sc12]

To a solution of 2-(4-(7-((5-(2-chloroacetamido)-2-methylpyridin-3-yl)carbamoyl)pyrazolo[5,1-b]thiazol-2-yl)-1H-pyrazol-1-yl)-2-methylpropyl 2-chloroacetate (121 mg, 0.21 mmol) and 2,2-dimethylpyrrolidine hydrochloride (72.69 mg, 0.54 mmol) in DMF (1 mL) was added K₂CO₃ (177.76 mg, 1.29 mmol). The reaction was heated at 60° C. for 4 days. LCMS indicated a mixture of the product and the bis-chloride displacement product. Water (a few drops) were added and heating was continued at 60° C. for 2 days. The reaction was filtered through an Acrodisc CR 13 mm syringe filter with a 0.45 um PTFE membrane. The mixture was purified by HPLC (H₂O/ACN, 0.16% NH₄OH, XBridge 19×100) to afford the product, N-(5-(2-(2,2-dimethylpyrrolidin-1-yl)acetamido)-2-methylpyridin-3-yl)-2-(1-(1-hydroxy-2-methylpropan-2-yl)-1H-pyrazol-4-yl)pyrazolo[5,1-b]thiazole-7-carboxamide, as a tan solid (65.5 mg). LCMS (ESI): mass calcd. for C₂₇H₃₄N₈O₃S, 550.2; m/z found, 551.3 [M+H]⁺. ¹H NMR (400 MHz, DMSO-d6) δ ppm 1.02 (s, 6H), 1.50 (s, 6H), 1.61-1.71 (m, 2H), 1.71-1.82 (m, 2H), 2.41 (s, 3H), 2.79 (t, J=1.00 Hz, 2H), 3.15 (s, 2H), 3.56-3.63 (m, 2H), 4.98-5.06 (m, 1H), 7.89 (s, 1H), 8.15 (d, J=1.00 Hz, 1H), 8.28 (s, 1H), 8.50 (s, 1H), 8.56 (s, 1H), 8.63 (d, J=1.00 Hz, 1H), 9.72 (s, 1H), 9.88 (s, 1H).

### Example 458. 2-(6,7-dihydro-4H-pyrazolo[5,1-c][1,4]oxazin-3-yl)-N-(5-(2-(2,2-dimethylpyrrolidin-1-yl)acetamido)-2-methylpyridin-3-yl)pyrazolo[5,1-b]thiazole-7-carboxamide

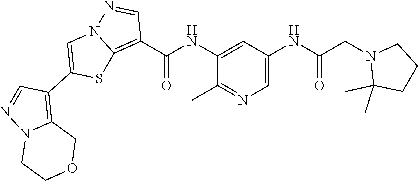 [SMILES: Cc1ncc(NC(=O)CN2CCCC2(C)C)cc1NC(=O)c1cnn2cc(-c3cnn4c3COCC4)sc12]

### Step a: tert-butyl (5-(2-(6,7-dihydro-4H₁-pyrazolo[5,1-c][1,4]oxazin-3-yl)pyrazolo[5,1-b]thiazole-7-carboxamido)-6-methylpyridin-3-yl)carbamate

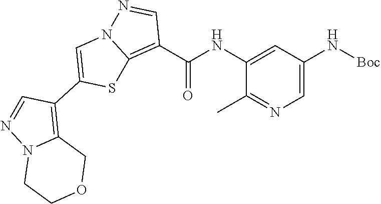 [SMILES: Cc1ncc(NC(=O)OC(C)(C)C)cc1NC(=O)c1cnn2cc(-c3cnn4c3COCC4)sc12]

To a mixture of tert-butyl (5-(2-bromopyrazolo[5,1-b]thiazole-7-carboxamido)-6-methylpyridin-3-yl)carbamate (400 mg, 0.88 mmol), 3-(4,4,5,5-tetramethyl-1,3,2-dioxaborolan-2-yl)-6,7-dihydro-4H-pyrazolo[5,1-c][1,4]oxazine (380 mg, 1.5 mmol) and Cs₂CO₃ (900 mg, 2.8 mmol) in dioxane (20 mL) and H₂O (5 mL) was added Pd(dppf)Cl₂·CH₂Cl₂ (220 mg, 0.27 mmol) at room temperature under N₂ atmosphere. The reaction mixture was purged with N₂ for 2 minutes. The reaction mixture was stirred at 100° C. for 16 hrs. The reaction mixture was concentrated in vacuo to give black solid, which was purified by column chromatography over silica gel (eluent: dichloromethane/methanol=100:0 to 95:5). The desired fractions were collected and the solvent was concentrated under vacuum to give desired product tert-butyl (5-(2-(6,7-dihydro-4H-pyrazolo[5,1-c][1,4]oxazin-3-yl)pyrazolo[5,1-b]thiazole-7-carboxamido)-6-methylpyridin-3-yl)carbamate (140 mg, 30%) as a pale yellow solid. LCMS (ESI): mass calcd. for C₂₃H₂₅N₇O₄S, 495.2; m/z found, 496.2 [M+H]⁺.

### Step b: N-(5-amino-2-methylpyridin-3-yl)-2-(6,7-dihydro-4H-pyrazolo[5,1-c][1,4]oxazin-3-yl)pyrazolo[5,1-b]thiazole-7-carboxamide

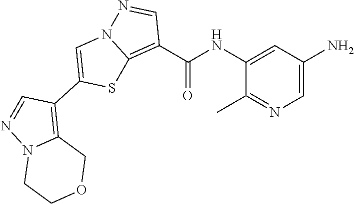 [SMILES: Cc1ncc(N)cc1NC(=O)c1cnn2cc(-c3cnn4c3COCC4)sc12]

To a solution of tert-butyl (5-(2-(6,7-dihydro-4H-pyrazolo[5,1-c][1,4]oxazin-3-yl)pyrazolo[5,1-b]thiazole-7-carboxamido)-6-methylpyridin-3-yl)carbamate (140 mg, 0.27 mmol) in DCM (1 mL) was added HCl/dioxane (0.7 mL, 2.8 mmol, 4 M) at room temperature. The reaction mixture was stirred at room temperature for 12 hrs. The reaction mixture was concentrated in vacuo to give crude of N-(5-amino-2-methylpyridin-3-yl)-2-(6,7-dihydro-4H-pyrazolo[5,1-c][1,4]oxazin-3-yl)pyrazolo[5,1-b]thiazole-7-carboxamide (120 mg, 85%) as a pale brown solid which was used in the next step without further purification. LCMS (ESI): mass calcd. for C₁₈H₁₇N₇O₂S, 395.1; m/z found, 396.1 [M+H]⁺.

### Step c: N-(5-(2-chloroacetamido)-2-methylpyridin-3-yl)-2-(6,7-dihydro-4H-pyrazolo[5,1-c][1,4]oxazin-3-yl)pyrazolo[5,1-b]thiazole-7-carboxamide

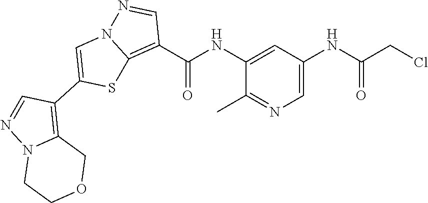 [SMILES: Cc1ncc(NC(=O)CCl)cc1NC(=O)c1cnn2cc(-c3cnn4c3COCC4)sc12]

To a mixture of N-(5-amino-2-methylpyridin-3-yl)-2-(6,7-dihydro-4H-pyrazolo[5,1-c][1,4]oxazin-3-yl)pyrazolo[5,1-b]thiazole-7-carboxamide hydrochloride (120 mg, 0.23 mmol) and NaHCO₃ (80 mg, 0.91 mmol) in DMF (5 mL) was added 2-chloroacetyl chloride (40 mg, 0.35 mmoL) dropwise at 0° C. Then the reaction was stirred at room temperature for 1.5 hrs. The reaction mixture was filtered. The filtrate was concentrated in vacuo to give crude target N-(5-(2-chloroacetamido)-2-methylpyridin-3-yl)-2-(6,7-dihydro-4H-pyrazolo[5,1-c][1,4]oxazin-3-yl)pyrazolo[5,1-b]thiazole-7-carboxamide (110 mg, 85%) as brown solid, which was used in the next step without further purification. LCMS (ESI): mass calcd. for C₂₀H₁₈ClN₇O₃S, 471.1; m/z found, 472.1 [M+H]⁺.

### Step d: 2-(6,7-dihydro-4H-pyrazolo[5,1-c][1,4]oxazin-3-yl)-N-(5-(2-(2,2-dimethylpyrrolidin-1-yl)acetamido)-2-methylpyridin-3-yl)pyrazolo[5,1-b]thiazole-7-carboxamide

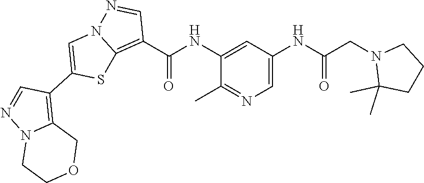 [SMILES: Cc1ncc(NC(=O)CN2CCCC2(C)C)cc1NC(=O)c1cnn2cc(-c3cnn4c3COCC4)sc12]

To a solution of N-(5-(2-chloroacetamido)-2-methylpyridin-3-yl)-2-(6,7-dihydro-4H-pyrazolo[5,1-c][1,4]oxazin-3-yl)pyrazolo[5,1-b]thiazole-7-carboxamide (110 mg, 0.19 mmol) and 2,2-dimethylpyrrolidine (70 mg, 0.69 mmol) in DMF (5 mL) was added K₂CO₃ (130 mg, 0.91 mmol) and NaI (40 mg, 0.267 mmol). The reaction mixture was stirred at 50° C. for 1.5 hrs. The reaction mixture was purified by preparative high-performance liquid chromatography over column: Phenomenex C18 80*40 mm*3 um to give the title compound 2-(6,7-dihydro-4H-pyrazolo[5,1-c][1,4]oxazin-3-yl)-N-(5-(2-(2,2-dimethylpyrrolidin-1-yl)acetamido)-2-methylpyridin-3-yl)pyrazolo[5,1-b]thiazole-7-carboxamide (42.1 mg, 40%) as a white solid. LCMS (ESI): mass calcd. for C₂₆H₃₀N₈O₃S, 534.2; m/z found, 535.2 [M+H]⁺. ¹H NMR (400 MHz, METHANOL-d₄) δ 8.63 (d, J=2.3 Hz, 1H), 8.41 (s, 1H), 8.26 (d, J=2.3 Hz, 1H), 8.10 (s, 1H), 7.84 (s, 1H), 5.07 (s, 2H), 4.29-4.15 (m, 4H), 3.28 (s, 2H), 2.92 (br t, J=7.0 Hz, 2H), 2.52 (s, 3H), 1.94-1.83 (m, 2H), 1.83-1.75 (m, 2H), 1.12 (s, 6H).

### Example 459. N-(5-((2-(2-azabicyclo[2.2.2]octan-2-yl)ethyl)carbamoyl)-2-methylpyridin-3-yl)-2-(6,7-dihydro-4H-pyrazolo[5,1-c][1,4]oxazin-3-yl)pyrazolo[5,1-b]thiazole-7-carboxamide

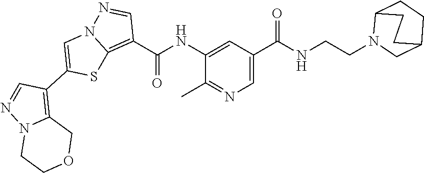 [SMILES: Cc1ncc(C(=O)NCCN2CC3CCC2CC3)cc1NC(=O)c1cnn2cc(-c3cnn4c3COCC4)sc12]

To a solution of 5-(2-(6,7-dihydro-4H-pyrazolo[5,1-c][1,4]oxazin-3-yl)pyrazolo[5,1-b]thiazole-7-carboxamido)-6-methylnicotinic acid (240 mg, 0.34 mmol), HATU (220 mg, 0.57 mmol) and N,N-diisopropylethylamine (0.27 mL, 1.6 mmol) in DMF (10 mL) was added 2-(2-azabicyclo[2.2.2]octan-2-yl)ethanamine (130 mg, 0.86 mmol). The resulting mixture was stirred at room temperature for 2 hrs and then concentrated under vacuum to give the crude product, which was purified by preparative high-performance liquid chromatography over column: Xtimate C18 150*40 mm*5 um to give the title compound N-(5-((2-(2-azabicyclo[2.2.2]octan-2-yl)ethyl)carbamoyl)-2-methylpyridin-3-yl)-2-(6,7-dihydro-4H-pyrazolo[5,1-c][1,4]oxazin-3-yl)pyrazolo[5,1-b]thiazole-7-carboxamide (37.4 mg, 17%, FA salt) as a white solid. LCMS (ESI): mass calcd. for C₂₈H₃₂N₈O₃S, 560.2; m/z found, 561.3 [M+H]⁺. ¹H NMR (400 MHz, METHANOL-d₄) δ 8.83 (s, 1H), 8.45 (s, 1H), 8.39 (s, 1H), 8.12 (s, 1H), 7.84 (s, 1H), 5.07 (s, 2H), 4.22 (br dd, J=5.1, 17.5 Hz, 4H), 3.78 (br t, J=5.6 Hz, 2H), 3.61 (br s, 1H), 3.46 (br t, J=5.8 Hz, 4H), 2.65 (s, 3H), 2.19 (br s, 2H), 2.02 (br s, 1H), 1.94-1.76 (m, 6H).

### Example 460. 2-(6,7-dihydro-5H-pyrazolo[5,1-b][1,3]oxazin-3-yl)-N-(5-((2-(2,2-dimethylpyrrolidin-1-yl)ethyl)carbamoyl)-2-methylpyridin-3-yl)pyrazolo[5,1-b]thiazole-7-carboxamide

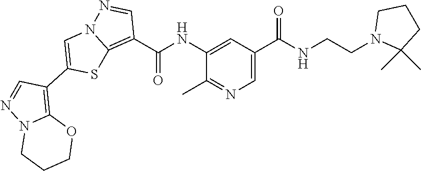 [SMILES: Cc1ncc(C(=O)NCCN2CCCC2(C)C)cc1NC(=O)c1cnn2cc(-c3cnn4c3OCCC4)sc12]

### Step a: methyl 5-(2-(6,7-dihydro-5H-pyrazolo[5,1-b][1,3]oxazin-3-yl)pyrazolo[5,1-b]thiazole-7-carboxamido)-6-methylnicotinate

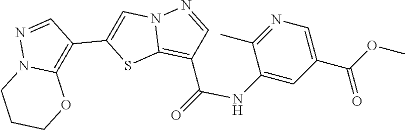 [SMILES: COC(=O)c1cnc(C)c(NC(=O)c2cnn3cc(-c4cnn5c4OCCC5)sc23)c1]

To a solution of methyl 5-(2-bromopyrazolo[5,1-b]thiazole-7-carboxamido)-6-methylnicotinate (200 mg, 0.51 mmol) in 1,4-dioxane/H₂O=4:1 (10 mL) was added 3-(4,4,5,5-tetramethyl-1,3,2-dioxaborolan-2-yl)-6,7-dihydro-5H-pyrazolo[5,1-b][1,3]oxazine (150 mg, 0.60 mmol), Cs₂CO₃ (500 mg, 1.5 mmol) and Pd(dppf)Cl₂·CH₂Cl₂ (100 mg, 0.12 mmol). The resultant mixture was stirred at 100° C. for 12 h. The mixture was concentrated under vacuum to afford methyl 5-(2-(6,7-dihydro-5H-pyrazolo[5,1-b][1,3]oxazin-3-yl)pyrazolo[5,1-b]thiazole-7-carboxamido)-6-methylnicotinate (600 mg, 50%) as a brown oil. The brown oil was used next step without purification. LCMS (ESI): mass calcd. for C₂₀H₁₈N₆O₄S, 438.5; m/z found, 439.2 [M+H]⁺.

### Step b: 5-(2-(6,7-dihydro-5H-pyrazolo[5,1-b][1,3]oxazin-3-yl)pyrazolo[5,1-b]thiazole-7-carboxamido)-6-methylnicotinic acid

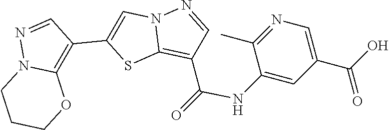 [SMILES: Cc1ncc(C(=O)O)cc1NC(=O)c1cnn2cc(-c3cnn4c3OCCC4)sc12]

To a solution of methyl 5-(2-(6,7-dihydro-5H-pyrazolo[5,1-b][1,3]oxazin-3-yl)pyrazolo[5,1-b]thiazole-7-carboxamido)-6-methylnicotinate (600 mg, 0.25 mmol) in THF/MeOH=1/1 (6 mL) was added lithium hydroxide hydrate (10 mg, 0.26 mmol) in H₂O (2 mL) and the reaction was stirred at 20° C. for 1 hour. The reaction mixture was poured into 200 mL of water carefully and the aqueous phase was washed with DCM (250 mL×3) and adjusted with 1N HCl(aq.) to pH=3. The resulting precipitate was collected by filtration. The solid was collected and dried in vacuo to give 5-(2-(6,7-dihydro-5H-pyrazolo[5,1-b][1,3]oxazin-3-yl)pyrazolo[5,1-b]thiazole-7-carboxamido)-6-methylnicotinic acid (240 mg, 60%) as a brown solid. LCMS (ESI): mass calcd. for C₁₉H₁₆N₆O₄S, 424.4 m/z found, 425.1 [M+H]⁺.

### Step c: 2-(6,7-dihydro-5H-pyrazolo[5,1-b][1,3]oxazin-3-yl)-N-(5-((2-(2,2-dimethylpyrrolidin-1-yl)ethyl)carbamoyl)-2-methylpyridin-3-yl)pyrazolo[5,1-b]thiazole-7-carboxamide

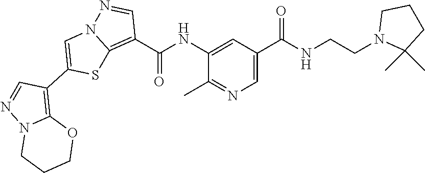 [SMILES: Cc1ncc(C(=O)NCCN2CCCC2(C)C)cc1NC(=O)c1cnn2cc(-c3cnn4c3OCCC4)sc12]

To a solution of 5-(2-(6,7-dihydro-5H-pyrazolo[5,1-b][1,3]oxazin-3-yl)pyrazolo[5,1-b]thiazole-7-carboxamido)-6-methylnicotinic acid (220 mg, 0.41 mmol) in DMF (5 mL) was added 2-(2,2-dimethylpyrrolidin-1-yl)ethanamine (90 mg, 0.62 mmol), DIEA (352 μL, 2.1 mmol) and HATU (240 mg, 0.64 mmol). The resulting mixture was stirred for 2 hours at room temperature. The mixture stirred at 25° C. for 1 h. Then the reaction was concentrated under vacuum to give the crude product, which was purified by preparative high-performance liquid chromatography over column: Boston Green ODS 150*30 mm*5 um to give the title compound 2-(6,7-dihydro-5H-pyrazolo[5,1-b][1,3]oxazin-3-yl)-N-(5-((2-(2,2-dimethylpyrrolidin-1-yl)ethyl)carbamoyl)-2-methylpyridin-3-yl)pyrazolo[5,1-b]thiazole-7-carboxamide (23 mg, 9%) as white solid. LCMS (ESI): mass calcd. for C₂₇H₃₂N₈O₃S. 548.7; m/z found, 549.3 [M+H]⁺. ¹H NMR (400 MHz, METHANOL-d₄) δ 8.81 (d, J=2.03 Hz, 1H), 8.55 (br s, 1H), 8.37 (d, J=2.03 Hz, 1H), 8.10 (s, 1H), 7.70 (s, 1H), 4.50-4.57 (m, 2H), 4.22 (t, J=6.14 Hz, 2H), 3.73 (br t, J=6.32 Hz, 2H), 3.39-3.54 (m, 2H), 3.18 (br s, 2H), 2.64 (s, 3H), 2.30-2.42 (m, 2H), 2.03-2.17 (m, 2H), 1.92-2.02 (m, 2H), 1.34 (s, 6H).

### Example 461. N-(5-((2-(5-azaspiro[3.4]octan-5-yl)ethyl)carbamoyl)-2-methylpyridin-3-yl)-2-(5,6-dihydro-4H-pyrrolo[1,2-b]pyrazol-3-yl)pyrazolo[5,1-b]thiazole-7-carboxamide

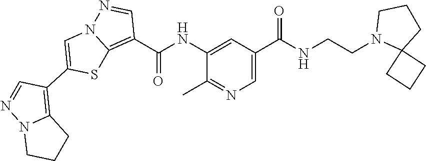 [SMILES: Cc1ncc(C(=O)NCCN2CCCC23CCC3)cc1NC(=O)c1cnn2cc(-c3cnn4c3CCC4)sc12]

To a solution of 5-(2-(5,6-dihydro-4H-pyrrolo[1,2-b]pyrazol-3-yl)pyrazolo[5,1-b]thiazole-7-carboxamido)-6-methylnicotinic acid (200 mg, 0.49 mmol), HATU (220 mg, 0.58 mmol) and N,N-diisopropylethylamine (0.34 mL, 2.1 mmol) in DMF (3 mL) was added 2-(5-azaspiro[3.4]octan-5-yl)ethanamine (100 mg, 0.65 mmol). The resulting mixture was stirred at room temperature for 2 h and then concentrated under vacuum to give the crude product, which was purified by column chromatography over silica gel (eluent: DCM/MeOH from 100:0 to 90:10) to give the crude product. The crude product was purified by preparative high-performance liquid chromatography over column: Welch Xtimate C18 150*30 mm*5 um to give the title compound N-(5-((2-(5-azaspiro[3.4]octan-5-yl)ethyl)carbamoyl)-2-methylpyridin-3-yl)-2-(5,6-dihydro-4H-pyrrolo[1,2-b]pyrazol-3-yl)pyrazolo[5,1-b]thiazole-7-carboxamide (30.3 mg, 11%) as a white solid. LCMS (ESI): mass calcd. for C₂₈H₃₂N₈O₂S, 544.6; m/z found, 545.4 [M+H]⁺. ¹H NMR (400 MHz, METHANOL-d₄) δ 8.80 (d, J=2.03 Hz, 1H), 8.42 (s, 1H), 8.33 (d, J=2.03 Hz, 1H), 8.19 (s, 1H), 7.86 (s, 1H), 4.21 (t, J=7.27 Hz, 2H), 3.58 (t, J=7.09 Hz, 2H), 3.12-3.16 (m, 2H), 2.76-2.86 (m, 6H), 2.63 (s, 3H), 2.25-2.32 (m, 2H), 1.98-2.03 (m, 2H), 1.81-1.88 (m, 2H), 1.67-1.75 (m, 4H).

### Example 462. 2-(1-(2-methoxyethyl)-1H-pyrazol-4-yl)-N-(2-methyl-5-(3-(1-methylpyrrolidin-2-yl)propanamido)pyridin-3-yl)pyrazolo[5,1-b]thiazole-7-carboxamide

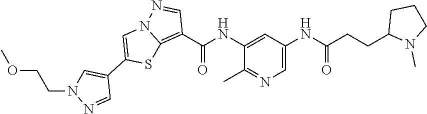 [SMILES: COCCn1cc(-c2cn3ncc(C(=O)Nc4cc(NC(=O)CCC5CCCN5C)cnc4C)c3s2)cn1]

### Step a: tert-butyl 2-(3-((5-(2-(1-(2-methoxyethyl)-1H-pyrazol-4-yl)pyrazolo[5,1-b]thiazole-7-carboxamido)-6-methylpyridin-3-yl)amino)-3-oxopropyl)pyrrolidine-1-carboxylate

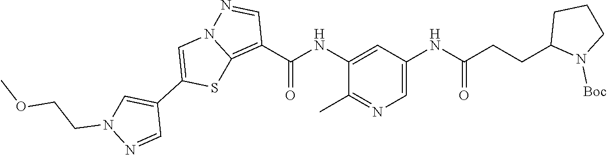 [SMILES: COCCn1cc(-c2cn3ncc(C(=O)Nc4cc(NC(=O)CCC5CCCN5C(=O)OC(C)(C)C)cnc4C)c3s2)cn1]

To a solution of N-(5-amino-2-methylpyridin-3-yl)-2-(1-(2-methoxyethyl)-1H-pyrazol-4-yl)pyrazolo[5,1-b]thiazole-7-carboxamide (300 mg, 0.61 mmol) in DMF (8 mL) was added 3-(1-(tertbutoxycarbonyl)pyrrole-din-2-yl)propanoic acid (200 mg, 0.82 mmol), N-(chloro(dimethylamino)methylene)-N-methylmethanaminium hexafluorophosphate(V) (400 mg, 1.4 mmol) and 1-methyl-1H-imidazole (400 mg, 4.9 mmol). The resulting mixture was stirred at 20° C. for 2 h before cooled to 25° C. The reaction mixture was filtered. The filtrate was concentrated under reduced pressure to give crude product, which was purified by column chromatography over silica gel (eluent: DCM:MeOH=4:1) to give tert-butyl 2-(3-((5-(2-(1-(2-methoxyethyl)-1H-pyrazol-4-yl)pyrazolo[5,1-b]thiazole-7-carboxamido)-6-methylpyridin-3-yl)amino)-3-oxopropyl)pyrrolidine-1-carboxylate (400 mg, 99%) as black solid. LCMS (ESI): mass calcd. for C₃₀H₃₈N₈O₅S, 622.7; m/z found, 623.3 [M+H]⁺.

### Step b: 2-(1-(2-methoxyethyl)-1H-pyrazol-4-yl)-N-(2-methyl-5-(3-(pyrrolidin-2-yl)propanamido)pyridin-3-yl)pyrazolo[5,1-b]thiazole-7-carboxamide

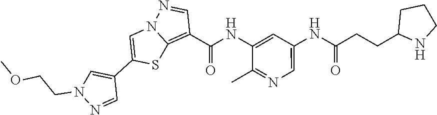 [SMILES: COCCn1cc(-c2cn3ncc(C(=O)Nc4cc(NC(=O)CCC5CCCN5)cnc4C)c3s2)cn1]

To a solution of tert-butyl 2-(3-((5-(2-(1-(2-methoxyethyl)-1H-pyrazol-4-yl)pyrazolo[5,1-b]thiazole-7-carboxamido)-6-methylpyridin-3-yl)amino)-3-oxopropyl)pyrrolidine-1-carboxylate (330 mg, 0.501 mmol) in DCM (7 mL) was added TFA (0.6 mL, 8.1 mmol) at room temperature for 2 hours. The resultant mixture was stirred at 20° C. for 1 hour. Then the reaction mixture was concentrated under reduced pressure to afford the crude product 2-(1-(2-methoxyethyl)-1H-pyrazol-4-yl)-N-(2-methyl-5-(3-(pyrrolidin-2-yl)propanamido)pyridin-3-yl)pyrazolo[5,1-b]thiazole-7-carboxamide (260 mg, crude) as white solid. LCMS (ESI): mass calcd. for C₂₅H₃₀N₈O₃S, 522.6; m/z found, 523.3 [M+H]⁺.

### Step c: 2-(1-(2-methoxyethyl)-1H-pyrazol-4-yl)-N-(2-methyl-5-(3-(1-methylpyrrolidin-2-yl)propanamido)pyridin-3-yl)pyrazolo[5,1-b]thiazole-7-carboxamide

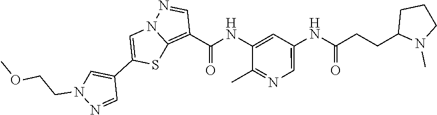 [SMILES: COCCn1cc(-c2cn3ncc(C(=O)Nc4cc(NC(=O)CCC5CCCN5C)cnc4C)c3s2)cn1]

To a solution of 2-(1-(2-methoxyethyl)-1H-pyrazol-4-yl)-N-(2-methyl-5-(3-(pyrrolidin-2-yl)propanamido)pyridin-3-yl)pyrazolo[5,1-b]thiazole-7-carboxamide (130 mg, 0.20 mmol) in MeOH (1 mL) was added Paraformaldehyde (70 mg, 0.72 mmol), acetic acid (20 μL, 0.35 mmol) and sodium cyanotrihydroborate (40 mg, 0.64 mmol). The resulting mixture was stirred at 25° C. for 2 h before cooling to room-temperature. The reaction mixture was filtered and the filtrate was concentrated under vacuum to give crude product, which was purified by preparative high-performance liquid chromatography over column: Phenomenex C18 80*40 mm*3 um to give 2-(1-(2-methoxyethyl)-1H-pyrazol-4-yl)-N-(2-methyl-5-(3-(1-methylpyrrolidin-2-yl)propanamido)pyridin-3-yl)pyrazolo[5,1-b]thiazole-7-carboxamide (43 mg, 40%) as a white solid. LCMS (ESI): mass calcd. for C₂₆H₃₂N₈O₃S, 536.6; m/z found, 537.3 [M+H]⁺. ¹H NMR (400 MHz, METHANOL-d₄) δ 8.55-8.59 (m, 1H), 8.38-8.42 (m, 1H), 8.23-8.27 (m, 2H), 8.08 (d, J=1.67 Hz, 1H), 7.87 (d, J=1.67 Hz, 1H), 4.32-4.41 (m, 2H), 3.79 (t, J=5.07 Hz, 2H), 3.36 (s, 3H), 3.13-3.23 (m, 1H), 2.34-2.60 (m, 10H), 2.09-2.28 (m, 2H), 1.79-1.91 (m, 2H), 1.53-1.71 (m, 2H).

### Example 463. N-(5-(3-(1-isopropylpyrrolidin-2-yl)propanamido)-2-methylpyridin-3-yl)-2-(1-(2-methoxyethyl)-1H-pyrazol-4-yl)pyrazolo[5,1-b]thiazole-7-carboxamide

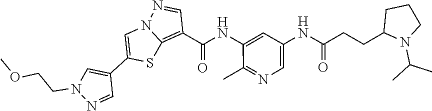 [SMILES: COCCn1cc(-c2cn3ncc(C(=O)Nc4cc(NC(=O)CCC5CCCN5C(C)C)cnc4C)c3s2)cn1]

To a solution of 2-(1-(2-methoxyethyl)-1H-pyrazol-4-yl)-N-(2-methyl-5-(3-(pyrrolidin-2-yl)propanamido)pyridin-3-yl)pyrazolo[5,1-b]thiazole-7-carboxamide (130 mg, 0.200 mmol) in MeOH (4 mL) was added propan-2-one (40 μL, 0.72 mmol), acetic acid (20 μL, 0.35 mmol) and sodium cyanotrihydroborate (40 mg, 0.64 mmol) at room temperature. The resulting mixture was stirred at 25° C. for 2 h before cooling to room temperature. The reaction mixture was filtered and the filtrate was concentrated under vacuum to give crude product, which was purified by preparative high-performance liquid chromatography over column: Boston Green ODS 150*30 mm*5 um to give N-(5-(3-(1-isopropylpyrrolidin-2-yl)propanamido)-2-methylpyridin-3-yl)-2-(1-(2-methoxyethyl)-1H-pyrazol-4-yl)pyrazolo[5,1-b]thiazole-7-carboxamide (46 mg, 40%) as white solid. LCMS (ESI): mass calcd. for C₂₈H₃₆N₈O₃S. 564.7; m/z found, 565.3 [M+H]⁺. ¹H NMR (400 MHz, METHANOL-d₄) δ 8.58 (d, J=2.26 Hz, 1H), 8.51-8.56 (m, 1H), 8.27-8.30 (m, 1H), 8.22-8.26 (m, 1H), 8.06-8.11 (m, 1H), 7.84-7.89 (m, 1H), 4.33-4.40 (m, 2H), 3.77-3.83 (m, 2H), 3.64-3.76 (m, 2H), 3.41-3.52 (m, 1H), 3.35-3.38 (m, 3H), 3.24-3.32 (m, 1H), 2.66-2.77 (m, 1H), 2.55-2.66 (m, 1H), 2.49-2.55 (m, 3H), 2.24-2.38 (m, 2H), 2.01-2.13 (m, 2H), 1.81-1.99 (m, 2H), 1.41-1.48 (m, 3H), 1.33-1.40 (m, 3H).

### Example 464. (S)—N-(5-(2-(1-isopropylpyrrolidin-3-yl)acetamido)-2-methylpyridin-3-yl)-2-(1-methyl-1H-pyrazol-4-yl)pyrazolo[5,1-b]thiazole-7-carboxamide

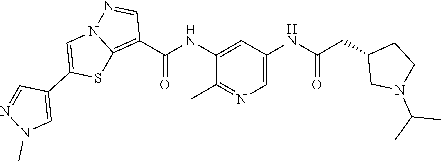 [SMILES: Cc1ncc(NC(=O)C[C@@H]2CCN(C(C)C)C2)cc1NC(=O)c1cnn2cc(-c3cnn(C)c3)sc12]

### Step a: (S)-tert-butyl 3-(2-((6-methyl-5-(2-(1-methyl-1H-pyrazol-4-yl)pyrazolo[5,1-b]thiazole-7-carboxamido)pyridin-3-yl)amino)-2-oxoethyl)pyrrolidine-1-carboxylate

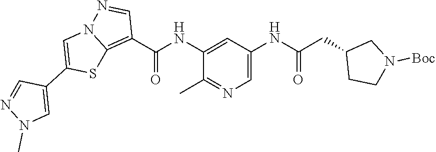 [SMILES: Cc1ncc(NC(=O)C[C@H]2CCN(C(=O)OC(C)(C)C)C2)cc1NC(=O)c1cnn2cc(-c3cnn(C)c3)sc12]

To a solution of methyl N-(5-amino-2-methylpyridin-3-yl)-2-(1-methyl-1H-pyrazol-4-yl)pyrazolo[5,1-b]thiazole-7-carboxamide (170 mg, 0.46 mmol) in DMF (3 mL) was added (S)-2-(1-(tert-butoxycarbonyl)pyrrolidin-3-yl)acetic acid (120 mg, 0.52 mmol), TCFH (270 mg, 0.97 mmol) and NMI (270 mg, 3.3 mmol). The mixture stirred at room temperature for 15 hours and then concentrated under vacuum to give the crude product, which was purified by column chromatography over silica gel (eluent: DCM/MeOH from 100:0 to 90:10) to give the crude product (S)-tert-butyl 3-(2-((6-methyl-5-(2-(1-methyl-1H-pyrazol-4-yl)pyrazolo[5,1-b]thiazole-7-carboxamido)pyridin-3-yl)amino)-2-oxoethyl)pyrrolidine-1-carboxylate (250 mg, 89%) as a white solid.

### Step b: (S)-2-(1-methyl-1H-pyrazol-4-yl)-N-(2-methyl-5-(2-(pyrrolidin-3-yl)acetamido)pyridin-3-yl)pyrazolo[5,1-b]thiazole-7-carboxamide

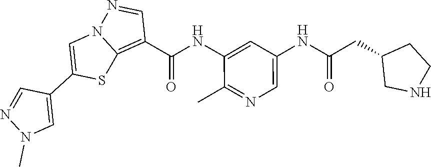 [SMILES: Cc1ncc(NC(=O)C[C@@H]2CCNC2)cc1NC(=O)c1cnn2cc(-c3cnn(C)c3)sc12]

To a solution of (S)-tert-butyl 3-(2-((6-methyl-5-(2-(1-methyl-1H-pyrazol-4-yl)pyrazolo[5,1-b]thiazole-7-carboxamido)pyridin-3-yl)amino)-2-oxoethyl)pyrrolidine-1-carboxylate (240 mg, 0.43 mmol) in DCM (5 mL) was added HCl/Dioxane (1.2 mL, 4.8 mmol, 4 M) at room temperature, The mixture was stirred at room temperature for 15 hours. The reaction mixture was concentrated under vacuum to give the crude product (S)-2-(1-methyl-1H-pyrazol-4-yl)-N-(2-methyl-5-(2-(pyrrolidin-3-yl)acetamido)pyridin-3-yl)pyrazolo[5,1-b]thiazole-7-carboxamide (250 mg, 121%) as a brown solid.

### Step c: (S)—N-(5-(2-(1-isopropylpyrrolidin-3-yl)acetamido)-2-methylpyridin-3-yl)-2-(1-methyl-1H-pyrazol-4-yl)pyrazolo[5,1-b]thiazole-7-carboxamide

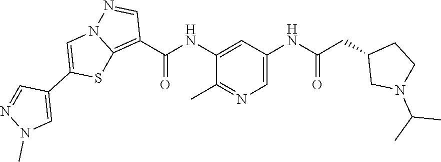 [SMILES: Cc1ncc(NC(=O)C[C@@H]2CCN(C(C)C)C2)cc1NC(=O)c1cnn2cc(-c3cnn(C)c3)sc12]

To a solution of (S)-2-(1-methyl-1H-pyrazol-4-yl)-N-(2-methyl-5-(2-(pyrrolidin-3-yl)acetamido)pyridin-3-yl)pyrazolo[5,1-b]thiazole-7-carboxamide (230 mg, 0.5 mmol) in MeOH (5 mL) was added propan-2-one (0.1 mL, 1.7 mmol) acetic acid (0.06 mL, 1 mmol) and NaBH₃CN (95 mg, 1.5 mmol). The mixture stirred at room temperature for 12 hours and then concentrated under vacuum to give the crude product, which was purified by preparative high-performance liquid chromatography over column: Welch Xtimate C18 150*30 mm*5 um to give the title compound (S)—N-(5-(2-(1-isopropylpyrrolidin-3-yl)acetamido)-2-methylpyridin-3-yl)-2-(1-methyl-1H-pyrazol-4-yl)pyrazolo[5,1-b]thiazole-7-carboxamide (57.9 mg, 23%) as a white solid. LCMS (ESI): mass calcd. for C₂₅H₃₀N₈O₂S, 506.6 m/z found, 507.3 [M+H]⁺. ¹H NMR (400 MHz, METHANOL-d₄) δ 8.57 (d, J=2.27 Hz, 1H), 8.41 (s, 1H), 8.25 (s, 1H), 8.24 (d, J=2.27 Hz, 1H), 8.05 (s, 1H), 7.85 (s, 1H), 3.97 (s, 3H), 2.80-2.87 (m, 1H), 2.66-2.75 (m, 2H), 2.54 (d, J=7.51 Hz, 3H), 2.51 (s, 3H), 2.35-2.43 (m, 1H), 2.11-2.20 (m, 1H), 1.56-1.64 (m, 1H), 1.25-1.38 (m, 1H), 1.16 (t, J=5.72 Hz, 6H).

### Example 465. (R)—N-(5-(2-(1-isopropylpyrrolidin-3-yl)acetamido)-2-methylpyridin-3-yl)-2-(1-methyl-1H-pyrazol-4-yl)pyrazolo[5,1-b]thiazole-7-carboxamide

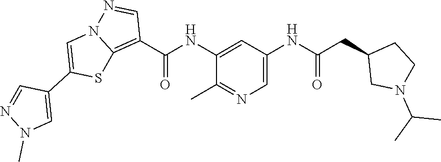 [SMILES: Cc1ncc(NC(=O)C[C@H]2CCN(C(C)C)C2)cc1NC(=O)c1cnn2cc(-c3cnn(C)c3)sc12]

### Step a: (R)-tert-butyl 3-(2-((6-methyl-5-(2-(1-methyl-1H-pyrazol-4-yl)pyrazolo[5,1-b]thiazole-7-carboxamido)pyridin-3-yl)amino)-2-oxoethyl)pyrrolidine-1-carboxylate

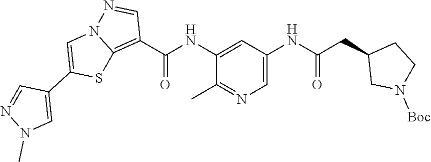 [SMILES: Cc1ncc(NC(=O)C[C@H]2CCN(C(=O)OC(C)(C)C)C2)cc1NC(=O)c1cnn2cc(-c3cnn(C)c3)sc12]

To a solution of N-(5-amino-2-methylpyridin-3-yl)-2-(1-methyl-1H-pyrazol-4-yl)pyrazolo[5,1-b]thiazole-7-carboxamide (200 mg, 0.493 mmol) in DMF (8 mL) was added (R)-2-(1-(tert-butoxycarbonyl)pyrrolidin-3-yl)acetic acid (130 mg, 0.545 mmol), 1-methyl-1H-imidazole (280 mg, 3.459 mmol) and N-(chloro(dimethylamino)methylene)-N-methylmethanaminium hexafluorophosphate(V) (280 mg, 0.987 mmol) at room temperature. The reaction mixture was stirred at room temperature for 2 hours. The reaction mixture was concentrated under vacuum to give a brown solid. The brown solid was purified by column chromatography over silica gel (gradient: DCM:MeOH from 100:0 to 80:20). The pure fractions were collected and concentrated to dryness in vacuo to give (R)-tert-butyl 3-(2-((6-methyl-5-(2-(1-methyl-1H-pyrazol-4-yl)pyrazolo[5,1-b]thiazole-7-carboxamido)pyridin-3-yl)amino)-2-oxoethyl)pyrrolidine-1-carboxylate (340 mg, 102%) as a brown solid. LCMS (ESI): mass for C₂₇H₃₂N₈O₄S: 564.7; m/z found: 565.3 [M+H]⁺.

### Step b: (R)-2-(1-methyl-1H-pyrazol-4-yl)-N-(2-methyl-5-(2-(pyrrolidin-3-yl)acetamido)pyridin-3-yl)pyrazolo[5,1-b]thiazole-7-carboxamide

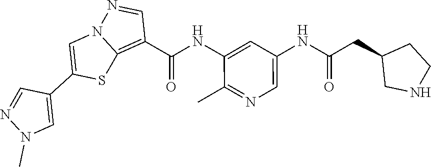 [SMILES: Cc1ncc(NC(=O)C[C@H]2CCNC2)cc1NC(=O)c1cnn2cc(-c3cnn(C)c3)sc12]

To a solution of (R)-tert-butyl 3-(2-((6-methyl-5-(2-(1-methyl-1H-pyrazol-4-yl)pyrazolo[5,1-b]thiazole-7-carboxamido)pyridin-3-yl)amino)-2-oxoethyl)pyrrolidine-1-carboxylate (320 mg, 0.487 mmol) in DCM (10 mL) was added HCl/Dioxane (1.3 mL, 5.2 mmol, 4 M) at room temperature. The reaction mixture was stirred at room temperature for 12 hours. The reaction mixture was concentrated in vacuum to give (R)-2-(1-methyl-1H-pyrazol-4-yl)-N-(2-methyl-5-(2-(pyrrolidin-3-yl)acetamido)pyridin-3-yl)pyrazolo[5,1-b]thiazole-7-carboxamide (320 mg, 114%) as a off-white solid. LCMS (ESI): mass for C₂₂H₂₄N₈O₂S: 464.5; m/z found: 465.2 [M+H]⁺.

### Step c: (R)—N-(5-(2-(1-isopropylpyrrolidin-3-yl)acetamido)-2-methylpyridin-3-yl)-2-(1-methyl-1H-pyrazol-4-yl)pyrazolo[5,1-b]thiazole-7-carboxamide

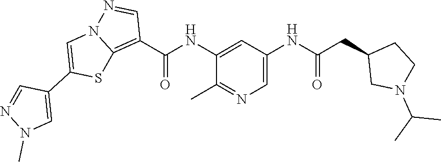 [SMILES: Cc1ncc(NC(=O)C[C@H]2CCN(C(C)C)C2)cc1NC(=O)c1cnn2cc(-c3cnn(C)c3)sc12]

To a solution of propan-2-one (90 μL, 1.626 mmol) in MeOH (15 mL) was added (R)-2-(1-methyl-1H-pyrazol-4-yl)-N-(2-methyl-5-(2-(pyrrolidin-3-yl)acetamido)pyridin-3-yl)pyrazolo[5,1-b]thiazole-7-carboxamide (300 mg, 0.520 mmol) and acetic acid (60 μL, 1.049 mmol) at 0° C. The mixture was stirred at room temperature for 0.5 hour. Then sodium cyanotrihydroborate (100 mg, 1.575 mmol) was added to the mixture. Then the mixture was stirred at 25° C. for 2 hours. The reaction mixture was concentrated under vacuum to give a brown solid. The brown solid was purified by preparative high-performance liquid chromatography over column: DAICEL CHIRALCEL OJ (250 mm*30 mm, 10 um) to give (R)—N-(5-(2-(1-isopropylpyrrolidin-3-yl)acetamido)-2-methylpyridin-3-yl)-2-(1-methyl-1H-pyrazol-4-yl)pyrazolo[5,1-b]thiazole-7-carboxamide (74.2 mg, 28%) as a white solid. The white solid off was separated by SFC over column: DAICEL CHIRALCEL OJ (250 mm*30 mm, 10 um). The pure fractions were collected and the solvent was evaporated under vacuum, lyophilized to dryness to obtain (R)—N-(5-(2-(1-isopropylpyrrolidin-3-yl) acetamido)-2-methylpyridin-3-yl)-2-(1-methyl-1H-pyrazol-4-yl)pyrazolo[5,1-b]thiazole-7-carboxamide (30.2 mg, 41%) as a white solid. LCMS (ESI): mass for C₂₅H₃₀N₈O₂S: 506.6; m/z found: 507.3 [M+H]⁺. ¹H NMR (400 MHz, METHANOL-d₄) δ 8.61-8.53 (m, 1H), 8.41 (s, 1H), 8.29-8.20 (m, 2H), 8.11-7.99 (m, 1H), 7.88-7.80 (m, 1H), 3.96 (s, 3H), 3.14-2.98 (m, 1H), 2.88-2.78 (m, 1H), 2.77-2.62 (m, 2H), 2.57-2.52 (m, 3H), 2.52-2.47 (m, 3H), 2.44-2.34 (m, 1H), 2.24-2.08 (m, 1H), 1.67-1.52 (m, 1H), 1.16 (t, J=5.7 Hz, 6H).

### Example 466. (S)—N-(5-(2-(1-isopropylpyrrolidin-3-yl)acetamido)-2-methylpyridin-3-yl)-2-(1-(2-methoxyethyl)-1H-pyrazol-4-yl)pyrazolo[5,1-b]thiazole-7-carboxamide

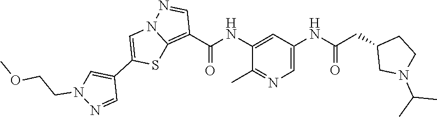 [SMILES: COCCn1cc(-c2cn3ncc(C(=O)Nc4cc(NC(=O)C[C@@H]5CCN(C(C)C)C5)cnc4C)c3s2)cn1]

### Step a: (S)-tert-butyl 3-(2-((5-(2-(1-(2-methoxyethyl)-1H-pyrazol-4-yl)pyrazolo[5,1-b]thiazole-7-carboxamido)-6-methylpyridin-3-yl)amino)-2-oxoethyl)pyrrolidine-1-carboxylate

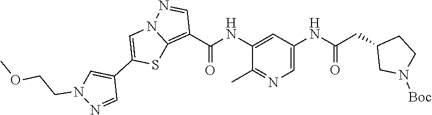 [SMILES: COCCn1cc(-c2cn3ncc(C(=O)Nc4cc(NC(=O)C[C@@H]5CCN(C(=O)OC(C)(C)C)C5)cnc4C)c3s2)cn1]

To a solution of N-(5-amino-2-methylpyridin-3-yl)-2-(1-(2-methoxyethyl)-1H-pyrazol-4-yl)pyrazolo[5,1-b]thiazole-7-carboxamide (150 mg, 0.33 mmol), (S)-2-(1-(tert-butoxycarbonyl)pyrrolidin-3-yl)acetic acid (85 mg, 0.37 mmol), 1-methyl-1H-imidazole (190 mg, 2.3 mmol) in DMF (5 mL) was added N-(chloro(dimethylamino)methylene)-N-methylmethanaminium hexafluorophosphate(V) (190 mg, 0.67 mmol) at room temperature. The resultant mixture was stirred at room temperature for 2 h. The reaction mixture was concentrated to give the crude product, which was purified by column chromatography over silica gel (eluent: petroleum ether:ethyl acetate=0:100) to give the title compound to give the title compound (S)-tert-butyl 3-(2-((5-(2-(1-(2-methoxyethyl)-1H-pyrazol-4-yl)pyrazolo[5,1-b]thiazole-7-carboxamido)-6-methylpyridin-3-yl)amino)-2-oxoethyl)pyrrolidine-1-carboxylate (180 mg, 89%) as a yellow solid. LCMS (ESI): mass calcd. for C₂₉H₃₆N₈O₅S, 608.712; m/z found, 609.3 [M+H]⁺.

### Step b: (S)-2-(1-(2-methoxyethyl)-1H-pyrazol-4-yl)-N-(2-methyl-5-(2-(pyrrolidin-3-yl)acetamido)pyridin-3-yl)pyrazolo[5,1-b]thiazole-7-carboxamide

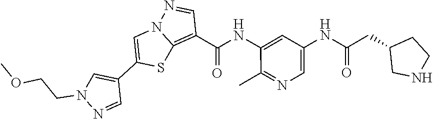 [SMILES: COCCn1cc(-c2cn3ncc(C(=O)Nc4cc(NC(=O)C[C@@H]5CCNC5)cnc4C)c3s2)cn1]

To a solution of tert-butyl (6-methyl-5-((1-methyl-6-((1-methyl-1H-pyrazol-4-yl)amino)-1H-pyrazolo[3,4-d]pyrimidin-3-yl)amino)pyridin-3-yl)carbamate (160 mg, 0.26 mmol) in dichloromethane (4 mL) was added HCl/Dioxane (1.0 mL, 4.0 mmol, 4 M). The resultant mixture was stirred at room temperature for 1 h. The reaction mixture was concentrated to give the title compound (S)-2-(1-(2-methoxyethyl)-1H-pyrazol-4-yl)-N-(2-methyl-5-(2-(pyrrolidin-3-yl)acetamido)pyridin-3-yl)pyrazolo[5,1-b]thiazole-7-carboxamide (140 mg, 86%) as a black solid. LCMS (ESI): mass calcd. for C₂₄H₂₈N₈O₃S, 508.596; m/z found, 509.2 [M+H]⁺.

### Step c: (S)—N-(5-(2-(1-isopropylpyrrolidin-3-yl)acetamido)-2-methylpyridin-3-yl)-2-(1-(2-methoxyethyl)-1H-pyrazol-4-yl)pyrazolo[5,1-b]thiazole-7-carboxamide

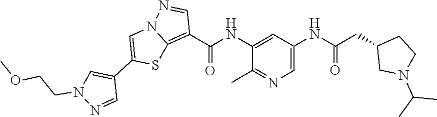 [SMILES: COCCn1cc(-c2cn3ncc(C(=O)Nc4cc(NC(=O)C[C@@H]5CCN(C(C)C)C5)cnc4C)c3s2)cn1]

To a solution of (S)-2-(1-(2-methoxyethyl)-1H-pyrazol-4-yl)-N-(2-methyl-5-(2-(pyrrolidin-3-yl)acetamido)pyridin-3-yl)pyrazolo[5,1-b]thiazole-7-carboxamide
(100 mg, 0.17 mmol), propan-2-one (30 mg, 0.52 mmol), acetic acid (21 mg, 0.34 mmol) in MeOH (5 mL) was added sodium cyanotrihydroborate (30 mg, 0.52 mmol). The mixture stirred at 25° C. for 1 h and then concentrated under vacuum to give the crude product, which was purified by preparative high-performance liquid chromatography over column: Boston Green ODS 150*30 mm*5 um to give the title compound (S)—N-(5-(2-(1-isopropylpyrrolidin-3-yl)acetamido)-2-methylpyridin-3-yl)-2-(1-(2-methoxyethyl)-1H-pyrazol-4-yl)pyrazolo[5,1-b]thiazole-7-carboxamide (40 mg, 60%) as a white solid. LCMS (ESI): mass calcd. for C₂₇H₃₄N₈O₃S, 550.676; m/z found, 551.3 [M+H]⁺. ¹H NMR (400 MHz, METHANOL-d₄) δ 8.57 (d, J=2.3 Hz, 1H), 8.41 (s, 1H), 8.25 (s, 2H), 8.07 (s, 1H), 7.86 (s, 1H), 4.36 (t, J=5.1 Hz, 2H), 3.79 (t, J=5.1 Hz, 2H), 3.71 (br s, 1H), 3.54-3.38 (m, 3H), 3.36 (s, 3H), 3.16 (br d, J=10.3 Hz, 1H), 2.92-2.78 (m, 1H), 2.77-2.62 (m, 2H), 2.52 (s, 3H), 2.42-2.29 (m, 1H), 1.91-1.77 (m, 1H), 1.40 (d, J=6.5 Hz, 6H).

### Example 467: N-(5-(2-(2-isopropylazetidin-1-yl)acetamido)-2-methylpyridin-3-yl)-2-(1-(2-methoxyethyl)-1H-pyrazol-4-yl)pyrazolo[5,1-b]thiazole-7-carboxamide

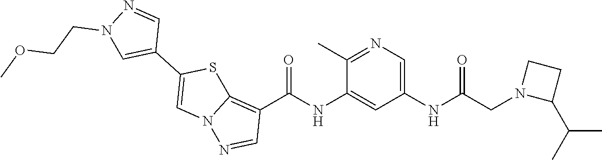

To a mixture of N-(5-(2-chloroacetamido)-2-methylpyridin-3-yl)-2-(1-(2-methoxyethyl)-1H-pyrazol-4-yl)pyrazolo[5,1-b]thiazole-7-carboxamide (60 mg, 0.13 mmol) and 2-isopropylazetidine, oxalic acid (28.74 mg, 0.15 mmol) in DMF (2 mL) was added K₂CO₃ (69.99 mg, 0.51 mmol). The reaction was heated at 50° C. for 17.5 h. The reaction was filtered through an Acrodisc CR 13 mm syringe filter with a 0.45 μm PTFE membrane. The reaction mixture was purified by HPLC (H₂O/ACN, 0.16% NH₄OH, XBridge 19×100) to afford the product, N-(5-(2-(2-isopropylazetidin-1-yl)acetamido)-2-methylpyridin-3-yl)-2-(1-(2-methoxyethyl)-1H-pyrazol-4-yl)pyrazolo[5,1-b]thiazole-7-carboxamide, as a tan solid (42.81 mg). LCMS (ESI): mass calcd. for C₂₆H₃₂N₈O₃S, 536.2; m/z found, 537.3 [M+H]⁺. ¹H NMR (400 MHz, DMSO-d6) δ ppm 0.79 (d, J=1.00 Hz, 3H), 0.85 (d, J=1.00 Hz, 3H), 1.68 (sxt, J=1.00 Hz, 1H), 1.81 (quin, J=1.00 Hz, 1H), 1.96 (q, J=1.00 Hz, 1H), 2.39 (s, 3H), 2.87 (q, J=1.00 Hz, 1H), 2.97 (q, J=1.00 Hz, 1H), 3.13 (d, J=1.00 Hz, 1H), 3.23 (s, 3H), 3.36 (d, J=1.00 Hz, 1H), 3.44 (t, J=1.00 Hz, 1H), 3.72 (t, J=1.00 Hz, 2H), 4.30 (t, J=1.00 Hz, 2H), 7.90 (s, 1H), 8.15 (d, J=1.00 Hz, 1H), 8.21 (s, 1H), 8.50 (s, 1H), 8.56 (d, J=1.00 Hz, 1H), 8.60 (s, 1H), 9.85 (s, 1H), 9.88 (s, 1H).

### Example 468. 2-(5,6-dihydro-4H-pyrrolo[1,2-b]pyrazol-3-yl)-N-(2-methyl-5-(2-(1-methylazetidin-3-yl)acetamido)pyridin-3-yl)pyrazolo[5,1-b]thiazole-7-carboxamide

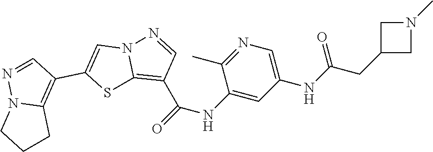 [SMILES: Cc1ncc(NC(=O)CC2CN(C)C2)cc1NC(=O)c1cnn2cc(-c3cnn4c3CCC4)sc12]

To a pale yellow solution of N-(5-(2-(azetidin-3-yl)acetamido)-2-methylpyridin-3-yl)-2-(5,6-dihydro-4H-pyrrolo[1,2-b]pyrazol-3-yl)pyrazolo[5,1-b]thiazole-7-carboxamide (24 mg, 0.041 mmol) and formalin (5 μL, 0.061 mmol) in MeOH (1.5 mL) was added NaBH(OAc)₃ (11 mg, 0.053 mmol) and the reaction was stirred at rt for 30 min. Excess formalin and NaBH(OAc)₃ were added and the reaction was stirred over the weekend. The reaction was concentrated, partitioned between EtOAc/sat'd NaHCO₃, and filtered of a small amount of a gummy white solid which was dissolved in DMF and purified by prep-HPLC, 29%-49% MeCN/water/10 mM NH₄OH to yield 2-(5,6-dihydro-4H-pyrrolo[1,2-b]pyrazol-3-yl)-N-(2-methyl-5-(2-(1-methylazetidin-3-yl)acetamido)pyridin-3-yl)pyrazolo[5,1-b]thiazole-7-carboxamide (3.2 mg, 16%) as a white solid. LCMS (ESI): mass calcd. for C₂₄H₂₆N₈O₂S, 490.6; m/z found, 491.2 [M+H]⁺. ¹H NMR (METHANOL-d4) δ: 8.52 (d, J=2.4 Hz, 1H), 8.37 (s, 1H), 8.20 (d, J=2.4 Hz, 1H), 8.13 (s, 1H), 7.82 (s, 1H), 4.18 (t, J=7.3 Hz, 2H), 3.55 (t, J=7.8 Hz, 2H), 3.07-3.14 (m, 2H), 3.03 (dd, J=7.8, 6.8 Hz, 2H), 2.88 (dquin, J=14.3, 7.2 Hz, 1H), 2.71-2.79 (m, 2H), 2.68 (d, J=7.8 Hz, 2H), 2.48 (s, 3H), 2.33 (s, 3H).

### Example 469. 2-(5,6-dihydro-4H-pyrrolo[1,2-b]pyrazol-3-yl)-N-(2-methyl-5-(((1-methylpyrrolidin-2-yl)methyl)carbamoyl)pyridin-3-yl)pyrazolo[5,1-b]thiazole-7-carboxamide

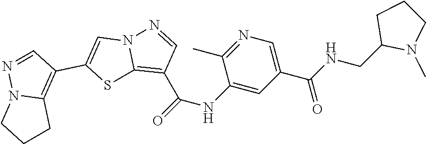 [SMILES: Cc1ncc(C(=O)NCC2CCCN2C)cc1NC(=O)c1cnn2cc(-c3cnn4c3CCC4)sc12]

### Step a: 5-(2-bromopyrazolo[5,1-b]thiazole-7-carboxamido)-6-methylnicotinic acid

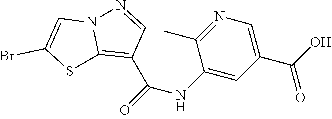

To a milky suspension of methyl 5-(2-bromopyrazolo[5,1-b]thiazole-7-carboxamido)-6-methylnicotinate (292.5 mg, 0.74 mmol) in 1,4-dioxane (10 mL) was added a clear solution of lithium hydroxide (54 mg, 2.22 mmol) in water (2 mL) to yield a hazy orange solution which was stirred at rt for 1 h. The reaction was acidified with 1N HCl (2.3 mL) and concentrated on the rotovap. The residue was stirred with water, filtered, and stripped solid down from MeCN to remove residual water to yield 5-(2-bromopyrazolo[5,1-b]thiazole-7-carboxamido)-6-methylnicotinic acid (234 mg, 83%) as a tan solid. LCMS (ESI): mass calcd. for C₁₃H₉BrN₄O₃S, 381.2; m/z found, 381.0/383.0 [M+H]⁺.

### Step b: 2-bromo-N-(2-methyl-5-(((1-methylpyrrolidin-2-yl)methyl)carbamoyl)pyridin-3-yl)pyrazolo[5,1-b]thiazole-7-carboxamide

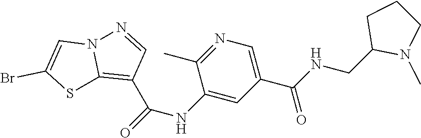 [SMILES: Cc1ncc(C(=O)NCC2CCCN2C)cc1NC(=O)c1cnn2cc(Br)sc12]

To a suspension of 5-(2-bromopyrazolo[5,1-b]thiazole-7-carboxamido)-6-methylnicotinic acid (108.5 mg, 0.28 mmol) and HATU (141 mg, 0.37 mmol) in DMF (2.5 mL) was added TEA (0.12 mL, 0.85 mmol). The mixture was stirred for 2 min, then (1-methylpyrrolidin-2-yl)methanamine (36.5 mg, 0.32 mmol) was added to yield a clear orange solution. After 10 min, the reaction was filtered and purified in two portions by prep-HPLC, 18%-38% MeCN/water/10 mM NH₄OH to yield 2-bromo-N-(2-methyl-5-(((1-methylpyrrolidin-2-yl)methyl)carbamoyl)pyridin-3-yl)pyrazolo[5,1-b]thiazole-7-carboxamide (102 mg, 68%) as a white solid. LCMS (ESI): mass calcd. for C₁₉H₂₁N₆O₂S, 477.4; m/z found, 477.0/479.0 [M+H]⁺.

### Step c: 2-(5,6-dihydro-4H-pyrrolo[1,2-b]pyrazol-3-yl)-N-(2-methyl-5-(((1-methylpyrrolidin-2-yl)methyl)carbamoyl)pyridin-3-yl)pyrazolo[5,1-b]thiazole-7-carboxamide

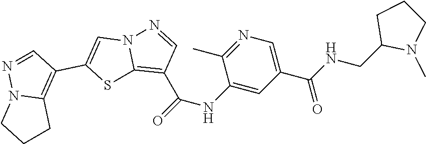 [SMILES: Cc1ncc(C(=O)NCC2CCCN2C)cc1NC(=O)c1cnn2cc(-c3cnn4c3CCC4)sc12]

The title compound was prepared from 2-bromo-N-(2-methyl-5-(((1-methylpyrrolidin-2-yl)methyl)carbamoyl)pyridin-3-yl)pyrazolo[5,1-b]thiazole-7-carboxamide according to the procedure of Example 372, substituting 3-(tetramethyl-1,3,2-dioxaborolan-2-yl)-4H,5H,6H-pyrrolo[1,2-b]pyrazole for 3-fluoro-5-(4,4,5,5-tetramethyl-1,3,2-dioxaborolan-2-yl)pyridin-2-amine (30 mg, 51%). LCMS (ESI): mass calcd. for C₂₅H₂₈N₈O₂S, 504.2; m/z found, 505.1 [M+H]⁺. ¹H NMR (METHANOL-d4) δ: 8.83 (s, 1H), 8.38-8.49 (m, 2H), 8.17 (d, J=1.0 Hz, 1H), 7.83 (s, 1H), 4.19 (t, J=7.3 Hz, 2H), 3.93-4.02 (m, 1H), 3.56-3.79 (m, 3H), 3.21 (dt, J=11.6, 7.9 Hz, 1H), 3.07-3.15 (m, 5H), 2.69-2.81 (m, 2H), 2.65 (d, J=1.5 Hz, 3H), 2.28-2.39 (m, 1H), 2.10-2.23 (m, 1H), 1.94-2.08 (m, 2H).

### Example 470. 2-(6,7-dihydro-5H-pyrazolo[5,1-b][1,3]oxazin-3-yl)-N-(2-methyl-5-(((1-methylpyrrolidin-2-yl)methyl)carbamoyl)pyridin-3-yl)pyrazolo[5,1-b]thiazole-7-carboxamide

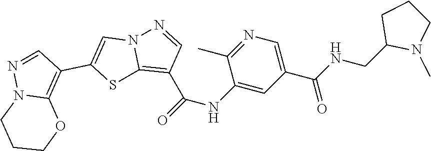 [SMILES: Cc1ncc(C(=O)NCC2CCCN2C)cc1NC(=O)c1cnn2cc(-c3cnn4c3OCCC4)sc12]

The title compound was prepared from 2-bromo-N-(2-methyl-5-(((1-methylpyrrolidin-2-yl)methyl)carbamoyl)pyridin-3-yl)pyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (19 mg, 30%). LCMS (ESI): mass calcd. for C₂₅H₂₈N₈O₃S, 520.2; m/z found, 521.2 [M+H]⁺. ¹H NMR (METHANOL-d4) δ: 8.83 (d, J=2.0 Hz, 1H), 8.42 (d, J=2.0 Hz, 1H), 8.39 (s, 1H), 8.09 (s, 1H), 7.68 (s, 1H), 4.49-4.56 (m, 2H), 4.20 (t, J=6.1 Hz, 2H), 3.94-4.01 (m, 1H), 3.58-3.78 (m, 3H), 3.17-3.25 (m, 2H), 3.09 (s, 3H), 2.64 (s, 3H), 2.28-2.40 (m, 3H), 2.10-2.23 (m, 1H), 1.94-2.09 (m, 2H).

### Example 471. N-(5-(2-(cyclobutylamino)acetamido)-2-methylpyridin-3-yl)-2-(5,6-dihydro-4H-pyrrolo[1,2-b]pyrazol-3-yl)pyrazolo[5,1-b]thiazole-7-carboxamide

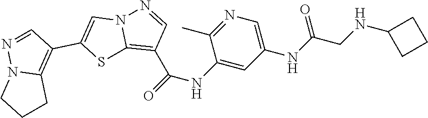 [SMILES: Cc1ncc(NC(=O)CNC2CCC2)cc1NC(=O)c1cnn2cc(-c3cnn4c3CCC4)sc12]

### Step a: 2-bromo-N-(5-(2-(cyclobutylamino)acetamido)-2-methylpyridin-3-yl)pyrazolo[5,1-b]thiazole-7-carboxamide

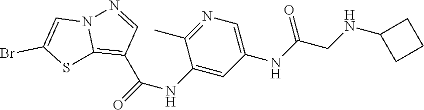 [SMILES: Cc1ncc(NC(=O)CNC2CCC2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of JNJ-86775533 step c replacing 3,3-dimethylazetidine hydrochloride with cyclobutylamine (103 mg, 56%). LCMS (ESI): mass calcd. for C₁₈H₁₉BrN₆O₂S, 463.4; m/z found, 463.0/465.0 [M+H]⁺.

### Step b: N-(5-(2-(cyclobutylamino)acetamido)-2-methylpyridin-3-yl)-2-(5,6-dihydro-4H-pyrrolo[1,2-b]pyrazol-3-yl)pyrazolo[5,1-b]thiazole-7-carboxamide

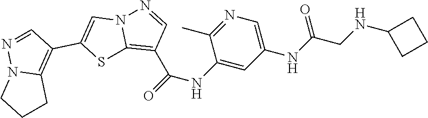 [SMILES: Cc1ncc(NC(=O)CNC2CCC2)cc1NC(=O)c1cnn2cc(-c3cnn4c3CCC4)sc12]

The title compound was prepared from 2-bromo-N-(5-(2-(cyclobutylamino)acetamido)-2-methylpyridin-3-yl)pyrazolo[5,1-b]thiazole-7-carboxamide according to the procedure of Example 372, substituting 3-(tetramethyl-1,3,2-dioxaborolan-2-yl)-4H,5H,6H-pyrrolo[1,2-b]pyrazole for 3-fluoro-5-(4,4,5,5-tetramethyl-1,3,2-dioxaborolan-2-yl)pyridin-2-amine (9 mg, 17%). LCMS (ESI): mass calcd. for C₂₄H₂₆N₈O₂S, 490.2; m/z found, 491.2 [M+H]⁺. ¹H NMR (METHANOL-d4) δ: 8.57 (d, J=2.4 Hz, 1H), 8.37 (s, 1H), 8.23 (d, J=2.4 Hz, 1H), 8.15 (s, 1H), 7.83 (s, 1H), 4.18 (t, J=7.3 Hz, 2H), 3.37 (s, 3H), 3.08-3.14 (m, 2H), 2.74 (quin, J=7.3 Hz, 2H), 2.49 (s, 3H), 2.15-2.27 (m, 2H), 1.61-1.91 (m, 4H).

### Example 472. N-(5-((2-(5-azaspiro[3.4]octan-5-yl)ethyl)carbamoyl)-2-methylpyridin-3-yl)-2-(6,7-dihydro-4H-pyrazolo[5,1-c][1,4]oxazin-3-yl)pyrazolo[5,1-b]thiazole-7-carboxamide

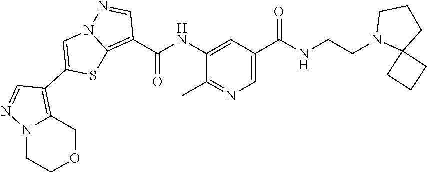 [SMILES: Cc1ncc(C(=O)NCCN2CCCC23CCC3)cc1NC(=O)c1cnn2cc(-c3cnn4c3COCC4)sc12]

To a solution of 5-(2-(6,7-dihydro-4H-pyrazolo[5,1-c][1,4]oxazin-3-yl)pyrazolo[5,1-b]thiazole-7-carboxamido)-6-methylnicotinic acid (270 mg, 0.21 mmol), HATU (145 mg, 0.38 mmol) and N,N-diisopropylethylamine (0.15 mL, 0.83 mmol) in DMF (10 mL) was added 2-(5-azaspiro[3.4]octan-5-yl)ethanamine (80 mg, 0.52 mmol). The resulting mixture was stirred at room temperature for 1 hr and then concentrated under vacuum to give the crude product, which was purified by preparative high-performance liquid chromatography over column: Xtimate C18 150*40 mm*5 um to give the title compound: N-(5-((2-(5-azaspiro[3.4]octan-5-yl)ethyl)carbamoyl)-2-methylpyridin-3-yl)-2-(6,7-dihydro-4H-pyrazolo[5,1-c][1,4]oxazin-3-yl)pyrazolo[5,1-b]thiazole-7-carboxamide (FA salt, 21.7 mg, 18%) as a white solid. LCMS (ESI): mass calcd. for C₂₈H₃₂N₈O₃S, 560.2; m/z found, 561.3 [M+H]⁺. ¹H NMR (400 MHz, METHANOL-d₄) δ 8.82 (d, J=2.0 Hz, 1H), 8.44 (s, 1H), 8.37 (d, J=2.0 Hz, 1H), 8.12 (s, 1H), 7.84 (s, 1H), 5.07 (s, 2H), 4.27-4.22 (m, 2H), 4.21-4.17 (m, 2H), 3.73 (t, J=6.5 Hz, 2H), 3.32-3.26 (m, 2H), 3.20 (br t, J=6.3 Hz, 2H), 2.64 (s, 3H), 2.53-2.43 (m, 2H), 2.23-2.17 (m, 2H), 2.04 (q, J=7.9 Hz, 2H), 1.99-1.93 (m, 2H), 1.92-1.83 (m, 2H).

### Example 473. (S)-2-(6,7-dihydro-4H-pyrazolo[5,1-c][1,4]oxazin-3-yl)-N-(2-methyl-5-(2-(2-methylpyrrolidin-1-yl)acetamido)pyridin-3-yl)pyrazolo[5,1-b]thiazole-7-carboxamide

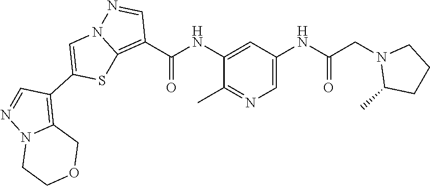 [SMILES: Cc1ncc(NC(=O)CN2CCC[C@@H]2C)cc1NC(=O)c1cnn2cc(-c3cnn4c3COCC4)sc12]

To a solution of N-(5-(2-chloroacetamido)-2-methylpyridin-3-yl)-2-(6,7-dihydro-4H-pyrazolo[5,1-c][1,4]oxazin-3-yl)pyrazolo[5,1-b]thiazole-7-carboxamide (100 mg, 0.20 mmol) and (S)-2-methylpyrrolidine (61 mg, 0.72 mmol) in DMF (5 mL) was added K₂CO₃ (132 mg, 0.96 mmol) and NaI (42 mg, 0.28 mmol). The reaction mixture was stirred at 50° C. for 1.5 hrs. The reaction mixture was purified by preparative high-performance liquid chromatography over column: Phenomenex C18 80*40 mm*3 um to give the title compound: (S)-2-(6,7-dihydro-4H-pyrazolo[5,1-c][1,4]oxazin-3-yl)-N-(2-methyl-5-(2-(2-methylpyrrolidin-1-yl)acetamido)pyridin-3-yl)pyrazolo[5,1-b]thiazole-7-carboxamide (39 mg, 37%) as a white solid. LCMS (ESI): mass calcd. for C₂₅H₂₈N₈O₃S, 520.2; m/z found, 521.2 [M+H]⁺. ¹H NMR (400 MHz, METHANOL-d₄) δ 8.62 (d, J=2.4 Hz, 1H), 8.42 (s, 1H), 8.26 (d, J=2.3 Hz, 1H), 8.10 (s, 1H), 7.84 (s, 1H), 5.08 (s, 2H), 4.27-4.22 (m, 2H), 4.21-4.16 (m, 2H), 3.57 (d, J=16.1 Hz, 1H), 3.30-3.24 (m, 1H), 3.15-3.09 (m, 1H), 2.66-2.56 (m, 1H), 2.52 (s, 3H), 2.42 (q, J=8.9 Hz, 1H), 2.10-1.98 (m, 1H), 1.93-1.76 (m, 2H), 1.61-1.47 (m, 1H), 1.18 (d, J=6.1 Hz, 3H).

### Example 474. N-(5-(2-(5-azaspiro[2.4]heptan-5-yl)acetamido)-2-methylpyridin-3-yl)-2-(6,7-dihydro-4H-pyrazolo[5,1-c][1,4]oxazin-3-yl)pyrazolo[5,1-b]thiazole-7-carboxamide

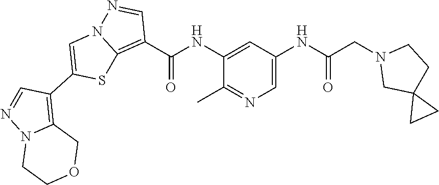 [SMILES: Cc1ncc(NC(=O)CN2CCC3(CC3)C2)cc1NC(=O)c1cnn2cc(-c3cnn4c3COCC4)sc12]

To a solution of N-(5-(2-chloroacetamido)-2-methylpyridin-3-yl)-2-(6,7-dihydro-4H-pyrazolo[5,1-c][1,4]oxazin-3-yl)pyrazolo[5,1-b]thiazole-7-carboxamide (110 mg, 0.19 mmol) and 5-azaspiro[2.4]heptane hydrochloride (91 mg, 0.68 mmol) in DMF (5 mL) was added K₂CO₃ (135 mg, 0.98 mmol) and NaI (40 mg, 0.27 mmol). The reaction mixture was stirred at 50° C. for 1.5 hrs. The reaction mixture was purified by preparative high-performance liquid chromatography over column: Phenomenex C18 80*40 mm*3 um to give the title compound: N-(5-(2-(5-azaspiro[2.4]heptan-5-yl)acetamido)-2-methylpyridin-3-yl)-2-(6,7-dihydro-4H-pyrazolo[5,1-c][1,4]oxazin-3-yl)pyrazolo[5,1-b]thiazole-7-carboxamide (38 mg, 37%) as a white solid. LCMS (ESI): mass calcd. for C₂₆H₂₈N₈O₃S, 532.2; m/z found, 533.2 [M+H]⁺. ¹H NMR (400 MHz, DMSO-d₆) δ 10.07 (br s, 1H), 9.94 (s, 1H), 8.65-8.50 (m, 2H), 8.42 (s, 1H), 8.18 (d, J=2.0 Hz, 1H), 7.90 (s, 1H), 5.05 (s, 2H), 4.21-4.14 (m, 2H), 4.13-4.08 (m, 2H), 2.84 (br t, J=6.5 Hz, 2H), 2.63 (s, 2H), 2.55-2.53 (m, 2H), 2.40 (s, 3H), 1.80 (t, J=6.8 Hz, 2H), 0.59-0.47 (m, 4H).

### Example 475. N-(5-(2-(1-azaspiro[3.3]heptan-1-yl)acetamido)-2-methylpyridin-3-yl)-2-(6,7-dihydro-4H-pyrazolo[5,1-c][1,4]oxazin-3-yl)pyrazolo[5,1-b]thiazole-7-carboxamide

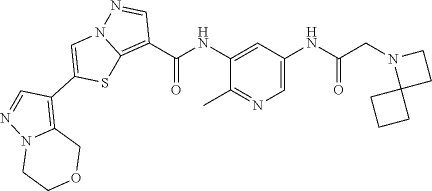 [SMILES: Cc1ncc(NC(=O)CN2CCC23CCC3)cc1NC(=O)c1cnn2cc(-c3cnn4c3COCC4)sc12]

To a solution of N-(5-(2-chloroacetamido)-2-methylpyridin-3-yl)-2-(6,7-dihydro-4H-pyrazolo[5,1-c][1,4]oxazin-3-yl)pyrazolo[5,1-b]thiazole-7-carboxamide (110 mg, 0.19 mmol) and 1-azaspiro[3.3]heptane hemioxalate (100 mg, 0.35 mmol) in DMF (5 mL) was added K₂CO₃ (135 mg, 0.98 mmol) and NaI (40 mg, 0.27 mmol). The reaction mixture was stirred at 50° C. for 1.5 hrs. The reaction mixture was purified by preparative high-performance liquid chromatography over column: Phenomenex C18 80*40 mm*3 um to give the title compound: N-(5-(2-(1-azaspiro[3.3]heptan-1-yl)acetamido)-2-methylpyridin-3-yl)-2-(6,7-dihydro-4H-pyrazolo[5,1-c][1,4]oxazin-3-yl)pyrazolo[5,1-b]thiazole-7-carboxamide (35 mg, 33%) as a white solid. LCMS (ESI): mass calcd. for C₂₆H₂₈N₈O₃S, 532.2; m/z found, 533.2 [M+H]⁺. ¹H NMR (400 MHz, DMSO-d₆) δ 10.01 (br s, 1H), 9.93 (s, 1H), 8.58 (d, J=2.1 Hz, 1H), 8.53 (s, 1H), 8.42 (s, 1H), 8.18 (d, J=2.0 Hz, 1H), 7.90 (s, 1H), 5.05 (s, 2H), 4.23-4.15 (m, 2H), 4.14-4.07 (m, 2H), 3.32-3.27 (m, 2H), 2.53 (s, 2H), 2.40 (s, 3H), 2.25 (br d, J=7.3 Hz, 4H), 1.95 (br s, 2H), 1.63-1.52 (m, 2H).

### Example 476. 2-(6,7-dihydro-4H-pyrazolo[5,1-c][1,4]oxazin-3-yl)-N-(5-(2-(3,3-dimethylazetidin-1-yl)acetamido)-2-methylpyridin-3-yl)pyrazolo[5,1-b]thiazole-7-carboxamide

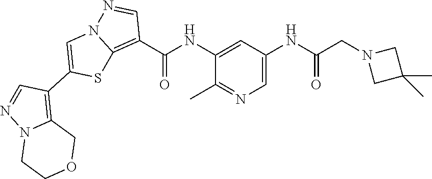 [SMILES: Cc1ncc(NC(=O)CN2CC(C)(C)C2)cc1NC(=O)c1cnn2cc(-c3cnn4c3COCC4)sc12]

To a solution of N-(5-(2-chloroacetamido)-2-methylpyridin-3-yl)-2-(6,7-dihydro-4H-pyrazolo[5,1-c][1,4]oxazin-3-yl)pyrazolo[5,1-b]thiazole-7-carboxamide (110 mg, 0.19 mmol) and 3,3-dimethylazetidine hydrochloride (88 mg, 0.72 mmol) in DMF (5 mL) was added K₂CO₃ (135 mg, 0.98 mmol) and NaI (40 mg, 0.27 mmol). The reaction mixture was stirred at 50° C. for 1.5 hrs. The reaction mixture was purified by preparative high-performance liquid chromatography over column: Phenomenex C18 80*40 mm*3 um to give the title compound: 2-(6,7-dihydro-4H-pyrazolo[5,1-c][1,4]oxazin-3-yl)-N-(5-(2-(3,3-dimethylazetidin-1-yl)acetamido)-2-methylpyridin-3-yl)pyrazolo[5,1-b]thiazole-7-carboxamide (36 mg, 36%) as a white solid. LCMS (ESI): mass calcd. for C₂₅H₂₈N₈O₃S, 520.2; m/z found, 521.2 [M+H]⁺. ¹H NMR (400 MHz, DMSO-d₆) δ 9.99 (br s, 1H), 9.93 (s, 1H), 8.60-8.50 (m, 2H), 8.42 (s, 1H), 8.15 (s, 1H), 7.90 (s, 1H), 5.06 (s, 2H), 4.18 (br d, J=5.0 Hz, 2H), 4.15-4.05 (m, 2H), 3.41 (br s, 2H), 3.17 (br s, 4H), 2.41 (s, 3H), 1.22 (s, 6H).

### Example 477. N-(5-((2-(2-azabicyclo[2.2.2]octan-2-yl)ethyl)carbamoyl)-2-methylpyridin-3-yl)-2-(6,7-dihydro-5H-pyrazolo[5,1-b][1,3]oxazin-3-yl)pyrazolo[5,1-b]thiazole-7-carboxamide

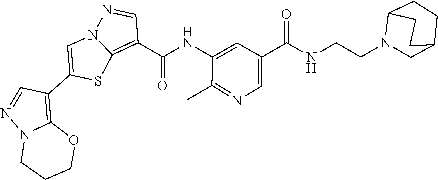 [SMILES: Cc1ncc(C(=O)NCCN2CC3CCC2CC3)cc1NC(=O)c1cnn2cc(-c3cnn4c3OCCC4)sc12]

To the mixture of 5-(2-(6,7-dihydro-5H-pyrazolo[5,1-b][1,3]oxazin-3-yl)pyrazolo[5,1-b]thiazole-7-carboxamido)-6-methylnicotinic acid (200 mg, 0.37 mmol), 2-(2-azabicyclo[2.2.2]octan-2-yl)ethanamine (70 mg, 0.4 mmol) and DIEA (0.24 mL, 1.4 mmol) in DMF (6 mL) was added HATU (164 mg, 0.4 mmol). The resulting mixture was stirred at 25° C. for 2 hours, and then concentrated under vacuum to give the crude product,
which was purified by preparative high-performance liquid chromatography over column: Xtimate C18 150*40 mm*5 um to give the title compound: N-(5-((2-(2-azabicyclo[2.2.2]octan-2-yl)ethyl)carbamoyl)-2-methylpyridin-3-yl)-2-(6,7-dihydro-5H-pyrazolo[5,1-b][1,3]oxazin-3-yl)pyrazolo[5,1-b]thiazole-7-carboxamide (21.3 mg, 9 0%) as a white solid. LCMS (ESI): mass calcd. for. C₂₈H₃₂N₈O₃S, 606.7; m/z found, 561.2 [M+H]⁺. ¹H NMR (400 MHz, METHANOL-d₄) δ 8.82 (d, J=2.0 Hz, 1H), 8.43-8.36 (m, 2H), 8.11 (s, 1H), 7.70 (s, 1H), 4.60 (s, 1H), 4.58-4.51 (m, 2H), 4.22 (t, J=6.1 Hz, 2H), 3.75 (t, J=6.1 Hz, 2H), 3.51 (br d, J=8.3 Hz, 1H), 3.44-3.36 (m, 3H), 2.65 (s, 3H), 2.41-2.33 (m, 2H), 2.17 (br s, 2H). 1.99 (br s. 1H). 1.89-1.76 (m, 6H).

### Example 478. N-(5-((2-(5-azaspiro[3.4]octan-5-yl)ethyl)carbamoyl)-2-methylpyridin-3-yl)-2-(6,7-dihydro-5H-pyrazolo[5,1-b][1,3]oxazin-3-yl)pyrazolo[5,1-b]thiazole-7-carboxamide

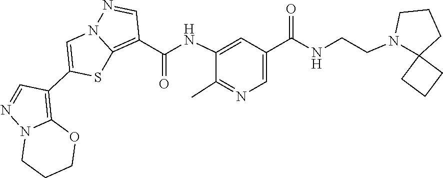 [SMILES: Cc1ncc(C(=O)NCCN2CCCC23CCC3)cc1NC(=O)c1cnn2cc(-c3cnn4c3OCCC4)sc12]

To the mixture of 5-(2-(6,7-dihydro-5H-pyrazolo[5,1-b][1,3]oxazin-3-yl)pyrazolo[5,1-b]thiazole-7-carboxamido)-6-methylnicotinic acid (260 mg, 0.32 mmol), 2-(5-azaspiro[3.4]octan-5-yl)ethanamine (117 mg, 0.76 mmol) and DIEA (220 μL, 1.3 mmol) in DMF (6 mL) was added HATU (286 mg, 0.75 mmol). The resulting mixture was stirred at 25° C. for 1 h. Then the reaction was concentrated under vacuum to give the crude product, which was purified by preparative high-performance liquid chromatography over column: Xtimate C18 150*40 mm*5 um to give the title compound: N-(5-((2-(5-azaspiro[3.4]octan-5-yl)ethyl)carbamoyl)-2-methylpyridin-3-yl)-2-(6,7-dihydro-5H-pyrazolo[5,1-b][1,3]oxazin-3-yl)pyrazolo[5,1-b]thiazole-7-carboxamide (24 mg, 12%) as a yellow solid. LCMS (ESI): mass calcd. for C₂₈H₃₂N₈O₃S. 560.7; m/z found, 561.3 [M+H]⁺. ¹H NMR (400 MHz, METHANOL-d₄) δ 8.81 (d, J=2.03 Hz, 1H), 8.40 (s, 1H), 8.36 (d, J=2.03 Hz, 1H), 8.10 (s, 1H), 7.70 (s, 1H), 4.50-4.58 (m, 2H), 4.22 (t, J=6.14 Hz, 2H), 3.70 (t, J=6.62 Hz, 2H), 3.21 (br t, J=6.79 Hz, 2H), 3.09-3.16 (m, 2H), 2.64 (s, 3H), 2.41-2.52 (m, 2H), 2.33-2.41 (m, 2H), 2.12-2.22 (m, 2H), 1.96-2.06 (m, 2H), 1.79-1.95 (m, 4H).

### Example 479. 2-(6,7-dihydro-5H-pyrazolo[5,1-b][1,3]oxazin-3-yl)-N-(5-((2-(3,3-dimethylazetidin-1-yl)ethyl)carbamoyl)-2-methylpyridin-3-yl)pyrazolo[5,1-b]thiazole-7-carboxamide

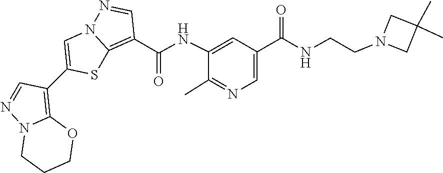 [SMILES: Cc1ncc(C(=O)NCCN2CC(C)(C)C2)cc1NC(=O)c1cnn2cc(-c3cnn4c3OCCC4)sc12]

To the mixture of 5-(2-(6,7-dihydro-5H-pyrazolo[5,1-b][1,3]oxazin-3-yl)pyrazolo[5,1-b]thiazole-7-carboxamido)-6-methylnicotinic acid (240 mg, 0.35 mmol), 2-(3,3-dimethylazetidin-1-yl)ethanamine (72 mg, 0.56 mmol) and DIEA (276 uL, 1.7 mmol) in DMF (8 mL) was added HATU (312 mg, 0.82 mmol). The resulting mixture was stirred at room temperature for 2 hours. Then the reaction was concentrated under vacuum to give the crude product, which was purified by preparative high-performance liquid chromatography over column: Xtimate C18 150*40 mm*5 um to give the title compound: 2-(6,7-dihydro-5H-pyrazolo[5,1-b][1,3]oxazin-3-yl)-N-(5-((2-(3,3-dimethylazetidin-1-yl)ethyl)carbamoyl)-2-methylpyridin-3-yl)pyrazolo[5,1-b]thiazole-7-carboxamide (29 mg, 14%) as a yellow solid. LCMS (ESI): mass calcd. for C₂₆H₃₀N₈O₃S, 534.6; m/z found, 535.3 [M+H]⁺. ¹H NMR (400 MHz, METHANOL-d₄) δ 8.81 (d, J=1.55 Hz, 1H), 8.41 (s, 1H), 8.37 (d, J=1.55 Hz, 1H), 8.10 (s, 1H), 7.70 (s, 1H), 4.51-4.58 (m, 2H), 4.22 (br t, J=6.08 Hz, 2H), 3.83 (s, 4H), 3.61 (br t, J=5.66 Hz, 2H), 3.26-3.32 (m, 2H), 2.64 (s, 3H), 2.32-2.41 (m, 2H), 1.37 (s, 6H).

### Example 480. N-(5-((2-(2-azabicyclo[2.2.2]octan-2-yl)ethyl)carbamoyl)-2-methylpyridin-3-yl)-2-(5,6-dihydro-4H-pyrrolo[1,2-b]pyrazol-3-yl)pyrazolo[5,1-b]thiazole-7-carboxamide

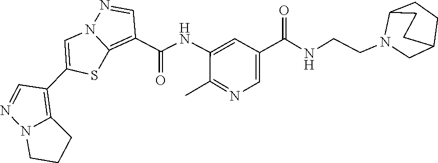 [SMILES: Cc1ncc(C(=O)NCCN2CC3CCC2CC3)cc1NC(=O)c1cnn2cc(-c3cnn4c3CCC4)sc12]

To the mixture of 5-(2-(5,6-dihydro-4H-pyrrolo[1,2-b]pyrazol-3-yl)pyrazolo[5,1-b]thiazole-7-carboxamido)-6-methylnicotinic acid (200 mg, 0.49 mmol), 2-(2-azabicyclo[2.2.2]octan-2-yl)ethanamine (100 mg, 0.65 mmol) and DIEA (340 μL, 2.1 mmol), in DMF (5 mL) was added HATU (220 mg, 0.58 mmol). The resulting mixture was stirred at room temperature for 2 hours. The mixture stirred at 25° C. for 1 h. Then the reaction was concentrated under vacuum to give the crude product, which were purified by preparative high-performance liquid chromatography over column: Xtimate C18 150*40 mm*5 um and separated by Supercritical Fluid Chromatography over column: DAICEL CHIRALCEL OD(250 mm*30 mm, 10 um) to give the title compound: N-(5-((2-(2-azabicyclo[2.2.2]octan-2-yl)ethyl)carbamoyl)-2-methylpyridin-3-yl)-2-(5,6-dihydro-4H-pyrrolo[1,2-b]pyrazol-3-yl)pyrazolo[5,1-b]thiazole-7-carboxamide (29 mg, 57%) as white solid. LCMS (ESI): mass calcd. for C₂₈H₃₂N₈O₂S, 544.7; m/z found, 545.3 [M+H]⁺. ¹H NMR (400 MHz, METHANOL-d₄) δ 8.80 (d, J=1.91 Hz, 1H), 8.42 (s, 1H), 8.33 (d, J=2.03 Hz, 1 H), 8.18 (s, 1H), 7.85 (s, 1H), 4.21 (t, J=7.27 Hz, 2H), 3.57 (t, J=6.97 Hz, 2H), 3.14 (t, J=7.15 Hz, 2H), 2.82-2.92 (m, 4H), 2.71-2.81 (m, 3H), 2.63 (s, 3H), 2.04 (br d, J=10.97 Hz, 2H), 1.70 (br s, 3H), 1.57-1.67 (m, 4H).

### Example 481. (S)—N-(5-(2-(1-isopropylpyrrolidin-3-yl)acetamido)-2-methylpyridin-3-yl)-2-(1-(2-methoxyethyl)-1H-pyrazol-4-yl)pyrazolo[5,1-b]thiazole-7-carboxamide

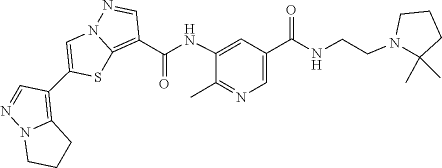 [SMILES: Cc1ncc(C(=O)NCCN2CCCC2(C)C)cc1NC(=O)c1cnn2cc(-c3cnn4c3CCC4)sc12]

### Step a: methyl 5-(2-(5,6-dihydro-4H-pyrrolo[1,2-b]pyrazol-3-yl)pyrazolo[5,1-b]thiazole-7-carboxamido)-6-methylnicotinate

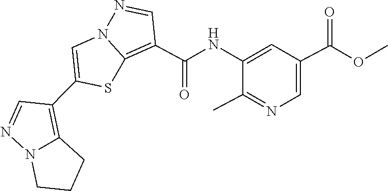 [SMILES: COC(=O)c1cnc(C)c(NC(=O)c2cnn3cc(-c4cnn5c4CCC5)sc23)c1]

To a solution of Methyl 5-(2-bromopyrazolo[5,1-b]thiazole-7-carboxamido)-6-methylnicotinate (600 mg, 1.5 mmol), 3-(4,4,5,5-tetramethyl-1,3,2-dioxaborolan-2-yl)-5,6-dihydro-4H-pyrrolo[1,2-b]pyrazole (403 mg, 1.7 mmol) and cesium carbonate (1.5 g, 4.5 mmol) in dioxane (20 mL) and H₂O (5 mL) was added Pd(dppf)Cl₂·CH₂Cl₂ (371 mg, 0.46 mmol) under an atmosphere of nitrogen. The resulting mixture was heated at 95° C. overnight. The reaction mixture was concentrated to give the crude product, which was purified by column chromatography over silica gel (eluent: dichloromethane:methanol=80:20) to give the title compound: methyl 5-(2-(5,6-dihydro-4H-pyrrolo[1,2-b]pyrazol-3-yl)pyrazolo[5,1-b]thiazole-7-carboxamido)-6-methylnicotinate (380 mg, 59%) as a brown solid. LCMS (ESI): mass calcd. for C₂₀H₁₈N₆O₃S, 422.46; m/z found, 423.1 [M+H]⁺.

### Step b: 5-(2-(5,6-dihydro-4H-pyrrolo[1,2-b]pyrazol-3-yl)pyrazolo[5,1-b]thiazole-7-carboxamido)-6-methylnicotinic acid

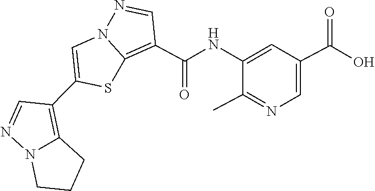 [SMILES: Cc1ncc(C(=O)O)cc1NC(=O)c1cnn2cc(-c3cnn4c3CCC4)sc12]

To a solution of Methyl 5-(2-(5,6-dihydro-4H-pyrrolo[1,2-b]pyrazol-3-yl)pyrazolo[5,1-b]thiazole-7-carboxamido)-6-methylnicotinate (350 mg, 0.83 mmol) in EtOH (3 mL) was added sodium hydroxide (1 mL, 2 mmol). The mixture was stirred at room temperature for 1 h. The mixture was adjusted to pH=3-4 with HCl (aq, 2 M), filtered and the residue was washed with H₂O (10 mL×3). The solid was dried under vacuum to give title compound: 5-(2-(5,6-dihydro-4H-pyrrolo[1,2-b]pyrazol-3-yl)pyrazolo[5,1-b]thiazole-7-carboxamido)-6-methylnicotinic acid (300 mg, 88%) as a black solid. LCMS (ESI): mass calcd. for C₁₉H₁₆N₆O₃S, 408.434; m/z found, 409.1 [M+H]⁺.

### Step c: 2-(5,6-dihydro-4H-pyrrolo[1,2-b]pyrazol-3-yl)-N-(5-((2-(2,2-dimethylpyrrolidin-1-yl)ethyl)carbamoyl)-2-methylpyridin-3-yl)pyrazolo[5,1-b]thiazole-7-carboxamide

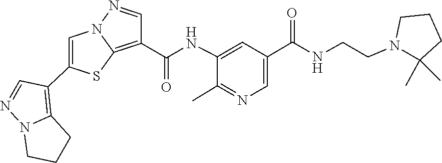 [SMILES: Cc1ncc(C(=O)NCCN2CCCC2(C)C)cc1NC(=O)c1cnn2cc(-c3cnn4c3CCC4)sc12]

To a solution of 5-((6-((1,3-dimethyl-1H-pyrazol-4-yl)amino)-1-methyl-1H-pyrazolo[3,4-d]pyrimidin-3-yl)amino)-6-methylnicotinic acid (100 mg, 0.25 mmol), 2-(2-azabicyclo[2.2.1]heptan-2-yl)ethanamine (39.6 mg, 0.28 mmol) and N-ethyl-N-isopropylpropan-2-amine (95 mg, 0.74 mmol) in N,N-dimethylformamide (5 mL) was added 2-(3H-[1,2,3]triazolo[4,5-b]pyridin-3-yl)-1,1,3,3-tetramethylisouronium hexafluorophosphate(V) (139 mg, 0.37 mmol). The mixture stirred at 25° C. for 1 h and then concentrated under vacuum to give the crude product, which was purified by preparative high-performance liquid chromatography over column: Welch Xtimate C18 150*30 mm*5 um to give the title compound: 2-(5,6-dihydro-4H-pyrrolo[1,2-b]pyrazol-3-yl)-N-(5-((2-(2,2-dimethylpyrrolidin-1-yl)ethyl)carbamoyl)-2-methylpyridin-3-yl)pyrazolo[5,1-b]thiazole-7-carboxamide (30.9 mg, 22%) as a white solid. LCMS (ESI): mass calcd. for C₂₇H₃₂N₈O₂S, 532.66; m/z found, 533 [M+H]⁺. ¹H NMR (400 MHz, METHANOL-d₄) δ 8.79 (d, J=1.8 Hz, 1H), 8.40-8.32 (m, 2H), 8.12 (s, 1H), 7.80 (s, 1H), 4.16 (t, J=7.3 Hz, 2H), 3.76 (t, J=6.3 Hz, 2H), 3.56 (br s, 2H), 3.26 (br t, J=5.8 Hz, 2H), 3.08 (t, J=7.3 Hz, 2H), 2.73 (quin, J=7.3 Hz, 2H), 2.61 (s, 3H), 2.15-1.98 (m, 4H), 1.37 (s, 6H).

### Example 482. 2-(5,6-dihydro-4H-pyrrolo[1,2-b]pyrazol-3-yl)-N-(5-((2-(3,3-dimethylazetidin-1-yl)ethyl)carbamoyl)-2-methylpyridin-3-yl)pyrazolo[5,1-b]thiazole-7-carboxamide

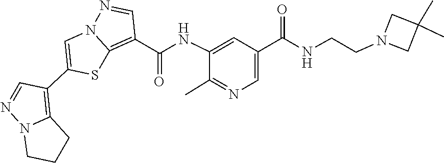 [SMILES: Cc1ncc(C(=O)NCCN2CC(C)(C)C2)cc1NC(=O)c1cnn2cc(-c3cnn4c3CCC4)sc12]

To a solution of 5-((6-((1,3-dimethyl-1H-pyrazol-4-yl)amino)-1-methyl-1H-pyrazolo[3,4-d]pyrimidin-3-yl)amino)-6-methylnicotinic acid (100 mg, 0.25 mmol), 2-(3,3-dimethylazetidin-1-yl)ethanamine (35.7 mg, 0.28 mmol), N-ethyl-N-isopropylpropan-2-amine (94.9 mg, 0.74 mmol) in N,N-dimethylformamide (5 mL) was added 2-(3H-[1,2,3]triazolo[4,5-b]pyridin-3-yl)-1,1,3,3-tetramethylisouronium hexafluorophosphate(V) (139 mg, 0.37 mmol). The mixture was stirred at 25° C. for 1 h and then concentrated under vacuum to give the crude product, which was purified by preparative high-performance liquid
chromatography over column: Phenomenex C18 75*30 mm*3 um to give the title compound: 2-(5,6-dihydro-4H-pyrrolo[1,2-b]pyrazol-3-yl)-N-(5-((2-(3,3-dimethylazetidin-1-yl)ethyl)carbamoyl)-2-methylpyridin-3-yl)pyrazolo[5,1-b]thiazole-7-carboxamide (21.5 mg, 17%) as a white solid. LCMS (ESI): mass calcd. for C₂₆H₃₀N₈O₂S, 518.634; m/z found, 519.3 [M+H]⁺. ¹H NMR (400 MHz, METHANOL-d₄) δ 8.79 (d, J=1.8 Hz, 1H), 8.40-8.32 (in, 2H), 8.12 (s, 1H), 7.80 (s, 1H), 4.16 (t, J=7.3 Hz, 2H), 3.76 (t, J=6.3 Hz, 2H), 3.56 (br s, 2H), 3.08 (t, J=7.3 Hz, 2H), 2.73 (quin, J=7.3 Hz, 2H), 2.61 (s, 3H), 2.15-1.98 (m, 4H), 1.37 (s, 6H).

### Example 483. N-(5-((2-(2,2-dimethylpyrrolidin-1-yl)ethyl)carbamoyl)-2-methylpyridin-3-yl)-2-(2-morpholinopyridin-4-yl)pyrazolo[5,1-b]thiazole-7-carboxamide

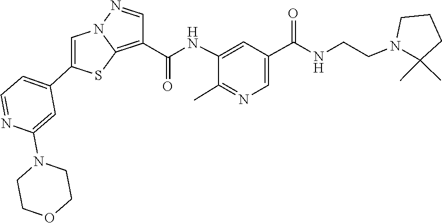 [SMILES: Cc1ncc(C(=O)NCCN2CCCC2(C)C)cc1NC(=O)c1cnn2cc(-c3ccnc(N4CCOCC4)c3)sc12]

### Step a: 4-(4-(4,4,5,5-tetramethyl-1,3,2-dioxaborolan-2-yl)pyridin-2-yl)morpholine

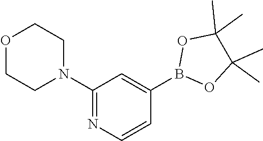 [SMILES: CC1(C)OB(c2ccnc(N3CCOCC3)c2)OC1(C)C]

To the mixture of 4-(4-bromopyridin-2-yl)morpholine (2 g, 8.2 mmol) in 1,4-dioxane/H₂O=4:1 (40 mL) was added 4,4,4′,4′,5,5,5′,5′-octamethyl-2,2′-bi(1,3,2-dioxaborolane) (2.5 g, 9.9 mmol) and potassium acetate (2.1 g, 21 mmol), then Pd(dppf)Cl₂·CH₂Cl₂ (1.2 g, 1.6 mmol) was added. The resulting mixture was heated at 90° C. and stirred for 12 hours under N₂. The mixture was concentrated under vacuum to afford 4-(4-(4,4,5,5-tetramethyl-1,3,2-dioxaborolan-2-yl)pyridin-2-yl)morpholine (2 g, 84%) as a brown oil. The brown oil was used next step without purification.

### Step b: methyl 6-methyl-5-(2-(2-morpholinopyridin-4-yl)pyrazolo[5,1-b]thiazole-7-carboxamido)nicotinate

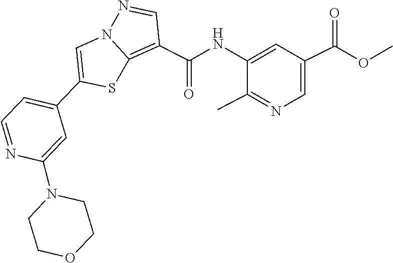 [SMILES: COC(=O)c1cnc(C)c(NC(=O)c2cnn3cc(-c4ccnc(N5CCOCC5)c4)sc23)c1]

To the mixture of methyl 5-(2-bromopyrazolo[5,1-b]thiazole-7-carboxamido)-6-methylnicotinate (220 mg, 0.56 mmol) in 1,4-dioxane/H₂O=4:1 (15 mL) was added 4-(4-(4,4,5,5-tetramethyl-1,3,2-dioxaborolan-2-yl)pyridin-2-yl)morpholine (880 mg, 3.0 mmol) and Cs₂CO₃ (550 mg, 1.7 mmol), then Pd(dppf)Cl₂·CH₂Cl₂ (110 mg, 0.14 mmol) was added. The resulting mixture was heated at 100° C. and stirred for 12 hours under N₂. The mixture cooled to room temperature and filtered. The filter cake was triturated with EtOAc/DCM/H₂O/MeOH=10/10/5/1 (20 mL) at room temperature for 30 min., then filtered. The solid was rinsed with 10 mL EtOAc, collected and dried to dryness in vacuo to give methyl 6-methyl-5-(2-(2-morpholinopyridin-4-yl)pyrazolo[5,1-b]thiazole-7-carboxamido)nicotinate (1 g, 94%) as a brown solid. LCMS (ESI): mass calcd. for C₂₀H₁₈N₆O₄S, 478.5; m/z found, 479.2 [M+H]⁺.

### Step c: 6-methyl-5-(2-(2-morpholinopyridin-4-yl)pyrazolo[5,1-b]thiazole-7-carboxamido)nicotinic acid

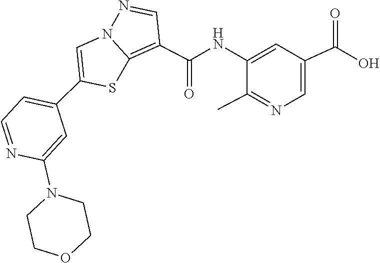 [SMILES: Cc1ncc(C(=O)O)cc1NC(=O)c1cnn2cc(-c3ccnc(N4CCOCC4)c3)sc12]

To a solution of methyl 6-methyl-5-(2-(2-morpholinopyridin-4-yl)pyrazolo[5,1-b]thiazole-7-carboxamido)nicotinate (0.98 g, 1.0 mmol) in THF/MeOH=1/1 (12 mL) was added lithium hydroxide hydrate (48 mg, 1.1 mmol) in H₂O (4 mL) and the reaction was stirred at 20° C. for 1 hour. The reaction mixture was poured into 50 mL of water carefully and the aqueous phase was washed with DCM (100 mL×3) and acidified with 1N HCl to pH=3. The resulting precipitate was collected by filtration and washed with 10 mL of H₂O. The solid was collected, dried in vacuo to give 6-methyl-5-(2-(2-morpholinopyridin-4-yl)pyrazolo[5,1-b]thiazole-7-carboxamido)nicotinic acid (240 mg, 60%) as a brown solid. LCMS (ESI): mass calcd. for C₂₂H₂₀N₆O₄S, 464.5 m/z found, 465.2 [M+H]⁺.

### Step d: N-(5-((2-(2,2-dimethylpyrrolidin-1-yl)ethyl)carbamoyl)-2-methylpyridin-3-yl)-2-(2-morpholinopyridin-4-yl)pyrazolo[5,1-b]thiazole-7-carboxamide

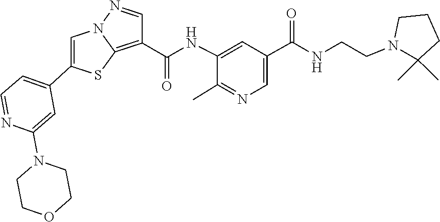 [SMILES: Cc1ncc(C(=O)NCCN2CCCC2(C)C)cc1NC(=O)c1cnn2cc(-c3ccnc(N4CCOCC4)c3)sc12]

To the mixture of 6-methyl-5-(2-(2-morpholinopyridin-4-yl)pyrazolo[5,1-b]thiazole-7-carboxamido)nicotinic acid (300 mg, 0.48 mmol), 2-(2,2-dimethylpyrrolidin-1-yl)ethanamine (105 mg, 0.74 mmol) and DIEA (315 μL, 1.9 mmol) in DMF (8 mL) was added HATU (225 mg, 0.59 mmol) was added. The resulting mixture was stirred at room temperature for 2 hours. Then the reaction was concentrated under vacuum to give the crude product, which was purified by preparative high-performance liquid chromatography over column: Xtimate C18 150*40 mm*5 um to give the title compound: N-(5-((2-(2,2-dimethylpyrrolidin-1-yl)ethyl)carbamoyl)-2-methylpyridin-3-yl)-2-(2-morpholinopyridin-4-yl)pyrazolo[5,1-b]thiazole-7-carboxamide (48 mg, 15%) as a white solid. LCMS (ESI): mass calcd. for C₃₀H₃₆N₈O₃S, 588.7; m/z found, 589.4 [M+H]⁺. ¹H NMR (400 MHz, METHANOL-d₄) δ 8.82 (d, J=2.03 Hz, 1H), 8.80 (s, 1H), 8.55 (br s, 1H), 8.38 (d, J=1.91 Hz, 1H), 8.21 (d, J=5.25 Hz, 1H), 7.06 (s, 1H), 7.00 (dd, J=5.25, 1.19 Hz, 1H), 3.80-3.90 (m, 4H), 3.73 (br t, J=6.32 Hz, 2H), 3.55-3.63 (m, 4H), 3.46 (br s, 2H), 3.17 (br s, 2H), 2.58-2.70 (m, 3H), 2.03-2.15 (m, 2H), 1.91-2.02 (m, 2H), 1.33 (s, 6H).

### Example 484. N-(5-((2-(2-azabicyclo[2.2.2]octan-2-yl)ethyl)carbamoyl)-2-methylpyridin-3-yl)-2-(2-morpholinopyridin-4-yl)pyrazolo[5,1-b]thiazole-7-carboxamide

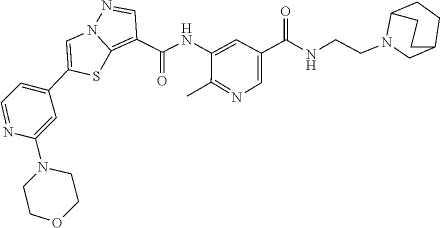 [SMILES: Cc1ncc(C(=O)NCCN2CC3CCC2CC3)cc1NC(=O)c1cnn2cc(-c3ccnc(N4CCOCC4)c3)sc12]

To a solution of 6-methyl-5-(2-(2-morpholinopyridin-4-yl)pyrazolo[5,1-b]thiazole-7-carboxamido)nicotinic acid (300 mg, 0.48 mmol), 2-(2-azabicyclo[2.2.2]octan-2-yl)ethanamine (90 mg, 0.58 mmol) and DIEA (315 μL, 1.9 mmol) in DMF (8 mL) was added HATU (225 mg, 0.59 mmol). The resulting mixture was stirred at room temperature for 2 hours. Then the reaction was concentrated under vacuum to give the crude product, which was purified by preparative high-performance liquid chromatography over column: Xtimate C18 150*40 mm*5 um to give the title compound: N-(5-((2-(2-azabicyclo[2.2.2]octan-2-yl)ethyl)carbamoyl)-2-methylpyridin-3-yl)-2-(2-morpholinopyridin-4-yl)pyrazolo[5,1-b]thiazole-7-carboxamide (52 mg, 16%) as a white solid. LCMS (ESI): mass calcd. for C₃₁H₃₆N₈O₃S, 600.7; m/z found, 601.3 [M+H]⁺. ¹H NMR (400 MHz, METHANOL-d₄) δ 8.83 (d, J=1.91 Hz, 1H), 8.80 (s, 1H), 8.56 (br s, 1H), 8.39 (d, J=1.91 Hz, 1H), 8.21 (d, J=5.36 Hz, 1H), 7.06 (s, 1H), 7.00 (d, J=5.36 Hz, 1H), 3.81-3.87 (m, 4H), 3.75 (br t, J=6.02 Hz, 2H), 3.56-3.62 (m, 4H), 3.49 (br s, 1H), 3.39 (br t, J=6.08 Hz, 4H), 2.65 (s, 3H), 2.17 (br s, 2H), 1.98 (br s, 1H), 1.72-1.91 (m, 6H).

### Example 485. (R)—N-(5-(2-(1-isopropylpyrrolidin-2-yl)acetamido)-2-methylpyridin-3-yl)-2-(1-(2-methoxyethyl)-1H-pyrazol-4-yl)pyrazolo[5,1-b]thiazole-7-carboxamide

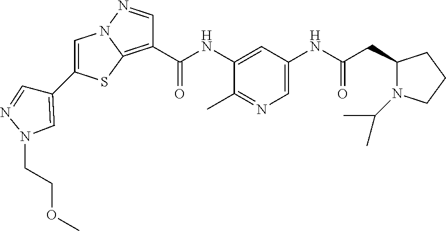 [SMILES: COCCn1cc(-c2cn3ncc(C(=O)Nc4cc(NC(=O)C[C@H]5CCCN5C(C)C)cnc4C)c3s2)cn1]

### Step a: (R)-tert-butyl 2-(2-((5-(2-(1-(2-methoxyethyl)-1H-pyrazol-4-yl)pyrazolo[5,1-b]thiazole-7-carboxamido)-6-methylpyridin-3-yl)amino)-2-oxoethyl)pyrrolidine-1-carboxylate

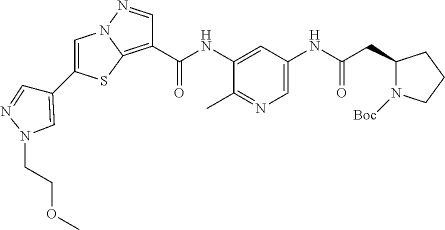 [SMILES: COCCn1cc(-c2cn3ncc(C(=O)Nc4cc(NC(=O)C[C@H]5CCCN5C(=O)OC(C)(C)C)cnc4C)c3s2)cn1]

To a solution of N-(5-amino-2-methylpyridin-3-yl)-2-(1-(2-methoxyethyl)-1H-pyrazol-4-yl)pyrazolo[5,1-b]thiazole-7-carboxamide (130 mg, 0.3 mmol), (R)-2-(1-(tert-butoxycarbonyl)pyrrolidin-2-yl)acetic acid (144 mg, 0.63 mmol) and N-(chloro(dimethylamino)methylene)-N-methylmethanaminium hexafluorophosphate(V) (195 mg, 0.69 mmol) in DMF (5 mL) was added 1-methyl-1H-imidazole (195 mg, 2.4 mmol). The resulting mixture was stirred at room temperature for 2 hours. The as colorless oil was purified by flash column chromatography over a 4 g silica gel (gradient: DCM:MeOH from 100:0 to 90:10). The filtrate was then concentrated to dryness under reduced pressure to afford the crude product (R)-tert-butyl2-(2-((5-(2-(1-(2-methoxyethyl)-1H-pyrazol-4-yl)pyrazolo[5,1-b]thiazole-7-carboxamido)-6-methylpyridin-3-yl)amino)-2-oxoethyl)pyrrolidine-1-carboxylate (160 mg, 77%) as a white solid. LCMS (ESI): mass calcd. for C₂₉H₃₆N₈O₅S, 608.7; m/z found, 609.5 [M+H]⁺.

### Step c: (R)-2-(1-(2-methoxyethyl)-1H-pyrazol-4-yl)-N-(2-methyl-5-(2-(pyrrolidin-2-yl)acetamido)pyridin-3-yl)pyrazolo[5,1-b]thiazole-7-carboxamide

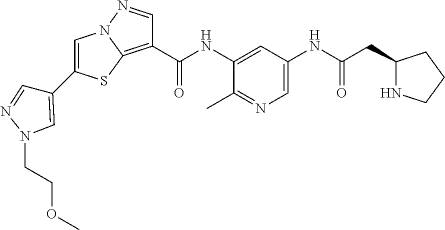 [SMILES: COCCn1cc(-c2cn3ncc(C(=O)Nc4cc(NC(=O)C[C@H]5CCCN5)cnc4C)c3s2)cn1]

To a solution of (R)-tert-butyl 2-(2-((5-(2-(1-(2-methoxyethyl)-1H-pyrazol-4-yl)pyrazolo[5,1-b]thiazole-7-carboxamido)-6-methylpyridin-3-yl)amino)-2-oxoethyl)pyrrolidine-1-carboxylate (140 mg, 0.183 mmol) in DCM/MeOH=1:1 (4 mL) was added HCl/dioxane (2 mL, 8 mmol, 4M) under N₂ atmosphere. The resulting mixture was stirred at room temperature for 12 hours. The filtrate was then concentrated to dryness under reduced pressure to afford the crude product (R)-2-(1-(2-methoxyethyl)-1H-pyrazol-4-yl)-N-(2-methyl-5-(2-(pyrrolidin-2-yl)acetamido)pyridin-3-yl)pyrazolo[5,1-b]thiazole-7-carboxamide (135 mg, crude, 1 M HCl) as a yellow oil. LCMS (ESI): mass calcd. for C₂₄H₂₈N₈O₃S, 545; m/z found, 509 [M+H]⁺

### Step d: (R)—N-(5-(2-(1-isopropylpyrrolidin-2-yl)acetamido)-2-methylpyridin-3-yl)-2-(1-(2-methoxyethyl)-1H-pyrazol-4-yl)pyrazolo[5,1-b]thiazole-7-carboxamide

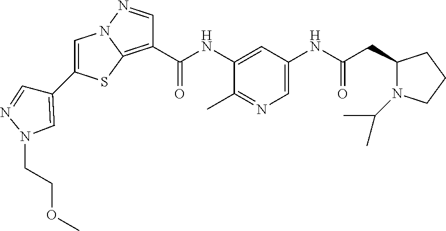 [SMILES: COCCn1cc(-c2cn3ncc(C(=O)Nc4cc(NC(=O)C[C@H]5CCCN5C(C)C)cnc4C)c3s2)cn1]

To a solution of (R)-2-(1-(2-methoxyethyl)-1H-pyrazol-4-yl)-N-(2-methyl-5-(2-(pyrrolidin-2-yl)acetamido)pyridin-3-yl)pyrazolo[5,1-b]thiazole-7-carboxamide (115 mg, 0.192 mmol), propan-2-one (95 μL, 1.7 mmol) and acetic acid (24 μL, 0.4 mmol) in MeOH (5 mL) was added Na(CN)BH₃ (52 mg, 0.8 mmol). The resulting mixture was stirred at room temperature for 2 hours. Then the reaction mixture concentrated under vacuum to give the crude product, which was purified by preparative high-performance liquid chromatography over column: Phenomenex C18 150*40 mm*5 um and by Supercritical Fluid Chromatography over column: Chiralpak AD-3 50ᵢÅ4.6 mm I.D., 3 um; to give the title compound: (R)—N-(5-(2-(1-isopropylpyrrolidin-2-yl)acetamido)-2-methylpyridin-3-yl)-2-(1-(2-methoxyethyl)-1H-pyrazol-4-yl)pyrazolo[5,1-b]thiazole-7-carboxamide (33.7 mg, 48%) as a white solid. LCMS (ESI): mass calcd. for C₂₇H₃₄N₈O₃S, 550.6; m/z found, 551.3 [M+H]⁺. ¹H NMR (400 MHz, METHANOL-d₄) δ 8.56 (d, J=2.3 Hz, 1H), 8.41 (s, 1H), 8.26 (s, 1H), 8.23 (d, J=2.3 Hz, 1H), 8.09 (s, 1H), 7.87 (s, 1H), 4.37 (t, J=5.1 Hz, 2H), 3.79 (t, J=5.2 Hz, 2H), 3.36 (s, 3H), 3.21-2.92 (m, 2H), 2.66 (br d, J=13.5 Hz, 2H), 2.52 (s, 4H), 2.13-1.99 (m, 1H), 1.83 (br d, J=7.2 Hz, 2H), 1.67 (br s, 1H), 1.31 (s, 1H), 1.23 (d, J=6.7 Hz, 3H), 1.11 (br d, J=6.1 Hz, 3H).

### Example 486. (R)—N-(5-(2-(1-isopropylpyrrolidin-3-yl)acetamido)-2-methylpyridin-3-yl)-2-(1-(2-methoxyethyl)-1H-pyrazol-4-yl)pyrazolo[5,1-b]thiazole-7-carboxamide

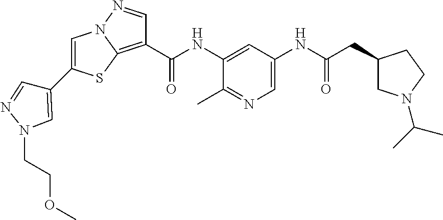 [SMILES: COCCn1cc(-c2cn3ncc(C(=O)Nc4cc(NC(=O)C[C@H]5CCN(C(C)C)C5)cnc4C)c3s2)cn1]

### Step a: (R)-tert-butyl 3-(2-((5-(2-(1-(2-methoxyethyl)-1H-pyrazol-4-yl)pyrazolo[5,1-b]thiazole-7-carboxamido)-6-methylpyridin-3-yl)amino)-2-oxoethyl)pyrrolidine-1-carboxylate

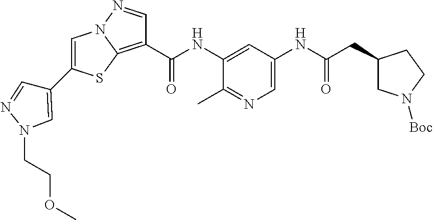 [SMILES: COCCn1cc(-c2cn3ncc(C(=O)Nc4cc(NC(=O)C[C@H]5CCN(C(=O)OC(C)(C)C)C5)cnc4C)c3s2)cn1]

To a solution of N-(5-amino-2-methylpyridin-3-yl)-2-(1-(2-methoxyethyl)-1H-pyrazol-4-yl)pyrazolo[5,1-b]thiazole-7-carboxamide (150 mg, 0.305 mmol) in DMF (3 mL) was added (R)-2-(1-(tert-butoxycarbonyl)pyrrolidin-3-yl)acetic acid (78 mg, 0.340 mmol) and 1-methyl-1H-imidazole (176 mg, 2.144 mmol), then N-(chloro(dimethylamino)methylene)-N-methylmethanaminium hexafluorophosphate(V) (172 mg, 0.613 mmol) was added at room temperature. The reaction mixture was stirred at room temperature for 2 hours. The reaction mixture was concentrated under vacuum to give a brown solid. The brown solid was subjected to column chromatography over 4 g silica gel (gradient: DCM:MeOH from 100:0 to 80:20). The pure fractions were collected and concentrated to dryness in vacuo to give (R)-tert-butyl 3-(2-((5-(2-(1-(2-methoxyethyl)-1H-pyrazol-4-yl)pyrazolo[5,1-b]thiazole-7-carboxamido)-6-methylpyridin-3-yl)amino)-2-oxoethyl)pyrrolidine-1-carboxylate (260 mg, 120%) as a brown solid. LCMS (ESI): mass for C₂₉H₃₆N₈O₅S: 608.7; m/z found: 609.3 [M+H]⁺.

### Step b: (R)-2-(1-(2-methoxyethyl)-1H-pyrazol-4-yl)-N-(2-methyl-5-(2-(pyrrolidin-3-yl)acetamido)pyridin-3-yl)pyrazolo[5,1-b]thiazole-7-carboxamide

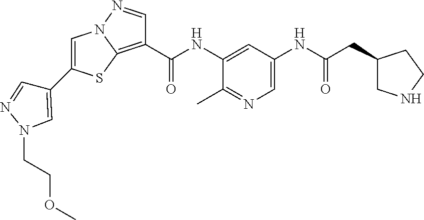 [SMILES: COCCn1cc(-c2cn3ncc(C(=O)Nc4cc(NC(=O)C[C@H]5CCNC5)cnc4C)c3s2)cn1]

To the mixture of (R)-tert-butyl 3-(2-((5-(2-(1-(2-methoxyethyl)-1H-pyrazol-4-yl)pyrazolo[5,1-b]thiazole-7-carboxamido)-6-methylpyridin-3-yl)amino)-2-oxoethyl)pyrrolidine-1-carboxylate (220 mg, 0.246 mmol) in DCM (20 mL) was added HCl/dioxane (0.7 mL, 2.8 mmol) at room temperature. The reaction mixture was stirred at room temperature for 12 hours. The reaction mixture turned cloudy. The reaction mixture was concentrated in vacuo to give (R)-2-(1-(2-methoxyethyl)-1H-pyrazol-4-yl)-N-(2-methyl-5-(2-(pyrrolidin-3-yl)acetamido)pyridin-3-yl)pyrazolo[5,1-b]thiazole-7-carboxamide as (260 mg, 120%) as an off-white solid. LCMS (ESI): mass for C₂₄H₂₈N₈O₃S: 508.6; m/z found: 509.3 [M+H]⁺.

### Step c: (R)—N-(5-(2-(1-isopropylpyrrolidin-3-yl)acetamido)-2-methylpyridin-3-yl)-2-(1-(2-methoxyethyl)-1H-pyrazol-4-yl)pyrazolo[5,1-b]thiazole-7-carboxamide

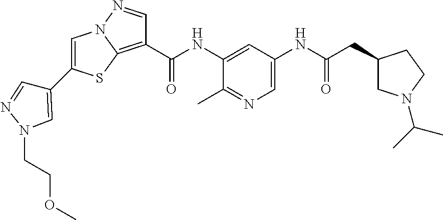 [SMILES: COCCn1cc(-c2cn3ncc(C(=O)Nc4cc(NC(=O)C[C@H]5CCN(C(C)C)C5)cnc4C)c3s2)cn1]

To a solution of propan-2-one (46 μL, 0.831 mmol) in MeOH (15 mL) was added (R)-2-(1-(2-methoxyethyl)-1H-pyrazol-4-yl)-N-(2-methyl-5-(2-(pyrrolidin-3-yl)acetamido)pyridin-3-yl)pyrazolo[5,1-b]thiazole-7-carboxamide (240 mg, 0.273 mmol) and acetic acid (32 μL, 0.560 mmol) at 0° C. The mixture was stirred at room temperature for 0.5 hour. Then sodium cyanotrihydroborate (52 mg, 0.827 mmol) was added to the mixture. Then the mixture was stirred at 25° C. for 2 hours. The reaction mixture was concentrated under vacuum to give a brown solid. The brown solid was purified by preparative high-performance liquid chromatography over column: Xtimate C18 150*40 mm*5 um. The pure fractions were collected and the solvent was evaporated under vacuum, lyophilized to dryness to give (R)—N-(5-(2-(1-isopropylpyrrolidin-3-yl)acetamido)-2-methylpyridin-3-yl)-2-(1-(2-methoxyethyl)-1H-pyrazol-4-yl)pyrazolo[5,1-b]thiazole-7-carboxamide (34 mg, 20%) as a pale brown solid.
LCMS (ESI): mass for C₂₇H₃₄N₈O₃S: 550.7; m/z found: 551.3 [M+H]+, ¹H NMR (400 MHz, METHANOL-d₄) δ 8.60-8.57 (m, 1H), 8.43-8.38 (m, 1H), 8.27 (s, 1H), 8.26-8.24 (m, 1H), 8.11-8.05 (m, 1H), 7.90-7.83 (m, 1H), 4.41-4.31 (m, 2H), 3.84-3.74 (m, 2H), 3.69 (br dd, J=8.7, 10.0 Hz, 1H), 3.51-3.38 (m, 3H), 3.36 (s, 3H), 3.20-3.06 (m, 1H), 2.90-2.78 (m, 1H), 2.77-2.60 (m, 2H), 2.56-2.48 (m, 3H), 2.43-2.29 (m, 1H), 1.90-1.76 (m, 1H), 1.45-1.36 (m, 6H).

### Example 487. N-(5-(1-((2,2-dimethylpyrrolidin-1-yl)methyl)cyclopropane-carboxamido)-2-methylpyridin-3-yl)-2-(1-(2-methoxyethyl)-1H-pyrazol-4-yl)pyrazolo[5,1-b]thiazole-7-carboxamide

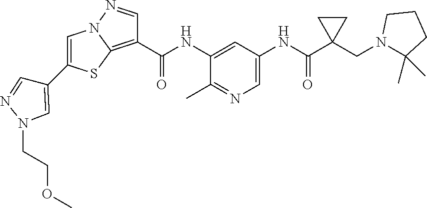 [SMILES: COCCn1cc(-c2cn3ncc(C(=O)Nc4cc(NC(=O)C5(CN6CCCC6(C)C)CC5)cnc4C)c3s2)cn1]

### Step a: methyl 1-((2,2-dimethylpyrrolidin-1-yl)methyl)cyclopropanecarboxylate

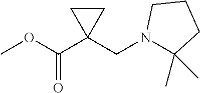

To a solution of 2,2-dimethylpyrrolidine (500 mg, 5.0 mmol), methyl 1-formylcyclopropanecarboxylate (1.3 g, 10 mmol), and acetic acid (0.43 mL, 5.3 mmol) in DCE (5 mL) was added sodium triacetyloxyboranuide (1.7 g, 8.0 mmol) and the mixture was stirred at room temperature for 2 hours. The as colourless oil was purified by flash column chromatography over a 12 g silica gel (gradient: DCM:MeOH from 100:0 to 90:10). The filtrate was then concentrated to dryness under reduced pressure to afford the crude product methyl 1-((2,2-dimethylpyrrolidin-1-yl)methyl)cyclopropanecarboxylate (440 mg, 41%) as a yellow oil. LCMS (ESI): mass calcd. for C₁₂H₂₁NO₂, 211; m/z found: 212 [M+H]⁺. ¹H NMR (400 MHz, CHLOROFORM-d) δ 3.68 (s, 3H), 3.26 (br t, J=7.5 Hz, 2H), 3.11 (s, 2H), 1.97-1.84 (m, 4H), 1.40-1.34 (m, 2H), 1.23 (s, 6H), 1.19-1.13 (m, 2H).

### Step b: 1-((2,2-dimethylpyrrolidin-1-yl)methyl)cyclopropanecarboxylic acid

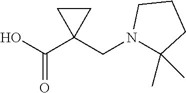

To a solution of methyl 1-((2,2-dimethylpyrrolidin-1-yl)methyl)cyclopropanecarboxylate (440 mg, 2.082 mmol) in mixture of Methanol:THF:H₂O 10=1:1:1 (21 mL) was added LiOH (97 mg, 2.312 mmol). The mixture was stirred at room temperature for 2 h and The filtrate was then concentrated to dryness under reduced pressure to afford the crude product 1-((2,2-dimethylpyrrolidin-1-yl)methyl)cyclopropanecarboxylic acid (400 mg, 97%) as a yellow oil. LCMS (ESI): mass calcd. for C₁₁H₁₉NO₂, 197; m/z found: 198 [M+H]⁺.

### Step c: N-(5-(1-((2,2-dimethylpyrrolidin-1-yl)methyl)cyclopropanecarboxamido)-2-methylpyridin-3-yl)-2-(1-(2-methoxyethyl)-1H-pyrazol-4-yl)pyrazolo[5,1-b]thiazole-7-carboxamide

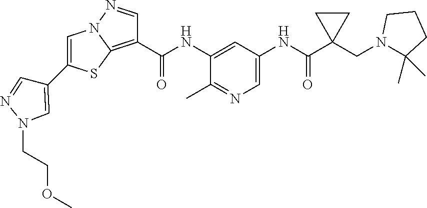 [SMILES: COCCn1cc(-c2cn3ncc(C(=O)Nc4cc(NC(=O)C5(CN6CCCC6(C)C)CC5)cnc4C)c3s2)cn1]

To the mixture of N-(5-amino-2-methylpyridin-3-yl)-2-(1-(2-methoxyethyl)-1H-pyrazol-4-yl)pyrazolo[5,1-b]thiazole-7-carboxamide (150 mg, 0.33 mmol), 1-((2,2-dimethyl-pyrrolidin-1-yl)methyl)cyclopropanecarboxylic acid (150 mg, 0.76 mmol) and N-(chloro(dimethylamino)methylene)-N-methylmethanaminium hexafluorophosphate(V) (276 mg, 0.98 mmol) in DMF (4 mL) was added 1-methyl-1H-imidazole (221 mg, 2.7 mmol). The resulting mixture was stirred at room temperature for 2 hours, which was purified by preparative high-performance liquid chromatography over column: Phenomenex Phenomenex C18 80*40 mm*3 um to give the title compound: N-(5-(1-((2,2-dimethylpyrrolidin-1-yl)methyl)cyclopropanecarboxamido)-2-methylpyridin-3-yl)-2-(1-(2-methoxyethyl)-1H-pyrazol-4-yl)pyrazolo[5,1-b]thiazole-7-carboxamide (29 mg, 14%) as a yellow solid. LCMS (ESI): mass calcd. for C₂₉H₃₆N₈O₃S, 576.7; m/z found: 577.3 [M+H]⁺. ¹H NMR (400 MHz, METHANOL-d₄) δ 8.54 (br s, 1H), 8.41 (s, 1H), 8.28-8.20 (m, 2H), 8.08 (s, 1H), 7.86 (s, 1H), 4.37 (t, J=5.1 Hz, 2H), 3.79 (t, J=5.1 Hz, 2H), 3.36 (s, 4H), 2.70 (br d, J=15.9 Hz, 1H), 2.52 (s, 4H), 2.01 (br s, 5H), 1.61-1.20 (m, 10H).

### Example 488. N-(5-(3-(2,2-dimethylpyrrolidin-1-yl)-2,2-dimethyl-propanamido)-2-methylpyridin-3-yl)-2-(1-(2-methoxyethyl)-1H-pyrazol-4-yl)pyrazolo[5,1-b]thiazole-7-carboxamide

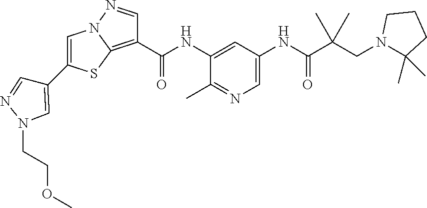 [SMILES: COCCn1cc(-c2cn3ncc(C(=O)Nc4cc(NC(=O)C(C)(C)CN5CCCC5(C)C)cnc4C)c3s2)cn1]

### Step a: methyl 3-(2,2-dimethylpyrrolidin-1-yl)-2,2-dimethylpropanoate

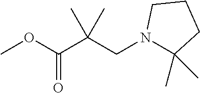

To a solution of methyl 2,2-dimethyl-3-oxopropanoate (300 mg, 2.3 mmol) in DCM (4 mL) was added 2,2-dimethylpyrrolidine (210 mg, 2.1 mmol), acetic acid (120 μL, 2.1 mmol) and sodium triacetoxyhydroborate (700 mg, 3.3 mmol) at room temperature. The resulting mixture was stirred at 25° C. for 2 h before cooling to room-temperature. The reaction mixture was filtered. The residue was washed with DCM (5 mL×3) and concentrated under reduced pressure to give crude product, which was purified by column chromatography over silica gel (eluent: DCM:MeOH=9:1) to give methyl 3-(2,2-dimethylpyrrolidin-1-yl)-2,2-dimethylpropanoate (150 mg, 33%) as a yellow oil. LCMS (ESI): mass calcd. for C₁₂H₂₃NO₂, 213.3; m/z found: 214.1 [M+H]⁺. ¹H NMR (400 MHz, CHLOROFORM-d) δ 3.63-3.80 (m, 3H), 2.74-2.95 (m, 2H), 2.56-2.70 (m, 2H), 1.71-1.84 (m, 2H), 1.55-1.70 (m, 2H), 1.23 (br s, 6H), 0.88-1.11 (m, 6H).

### Step b: 3-(2,2-dimethylpyrrolidin-1-yl)-2,2-dimethylpropanoic acid

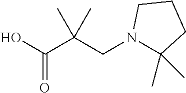

To a solution of methyl 3-(2,2-dimethylpyrrolidin-1-yl)-2,2-dimethylpropanoate (150 mg, 0.70 mmol) in THF/MeOH=1/1 (3 mL) was added lithium hydroxide hydrate (50 mg, 1.2 mmol) in H₂O (1 mL), and the resulting mixture was stirred at 20° C. for 1 h. After cooled to 0° C., the reaction mixture was quenched with water (0.25 mL) and filtered. The filtrate was concentrated to dryness under reduced pressure to afford the crude product 3-(2,2-dimethylpyrrolidin-1-yl)-2, 2-dimethylpropanoic acid (130 mg, 93%) as a white solid. LCMS (ESI): mass calcd. for C_{II}H₂₁NO₂, 199.2; m/z found: 200.2 [M+H]⁺.

### Step c: N-(5-(3-(2,2-dimethylpyrrolidin-1-yl)-2,2-dimethylpropanamido)-2-methylpyridin-3-yl)-2-(1-(2-methoxyethyl)-1H-pyrazol-4-yl)pyrazolo[5,1-b]thiazole-7-carboxamide

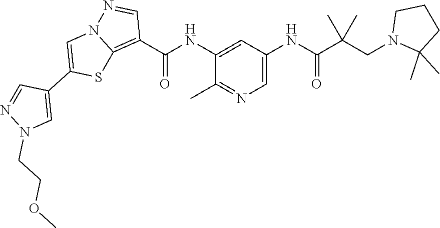 [SMILES: COCCn1cc(-c2cn3ncc(C(=O)Nc4cc(NC(=O)C(C)(C)CN5CCCC5(C)C)cnc4C)c3s2)cn1]

To the mixture of N-(5-amino-2-methylpyridin-3-yl)-2-(1-(2-methoxyethyl)-1H-pyrazol-4-yl)pyrazolo[5,1-b]thiazole-7-carboxamide (120 mg, 0.25 mmol), 3-(2,2-dimethylpyrrolidin-1-yl)-2,2-dimethylpropanoic acid (60 mg, 0.30 mmol) and N-(chloro(dimethylamino)methylene)-N-methylmethanaminium hexafluorophosphate(V) (250 mg, 0.90 mmol) in DMF (10 mL) was added 1-methyl-1H-imidazole (180 mg, 2.192 mmol). The mixture stirred at room temperature for 2 h and then concentrated under vacuum to give the crude product, which was purified by preparative high-performance liquid chromatography over column: Phenomenex C18 80*40 mm*3 um to give N-(5-(3-(2,2-dimethylpyrrolidin-1-yl)-2,2-dimethylpropanamido)-2-methylpyridin-3-yl)-2-(1-(2-methoxyethyl)-1H-pyrazol-4-yl)pyrazolo[5,1-b]thiazole-7-carboxamide (13 mg, 9%) as a white solid. LCMS (ESI): mass calcd. for C₂₉H₃₈N₈O₃S, 578.7 m/z found, 579.2 [M+H]⁺. ¹H NMR (400 MHz, METHANOL-d₄) δ 8.55 (d, J=2.15 Hz, 1H), 8.41 (s, 1H), 8.26 (s, 1H), 8.23-8.25 (m, 1H), 8.08-8.10 (m, 1H), 7.86-7.88 (m, 1H), 4.37 (t, J=5.13 Hz, 2H), 3.79 (t, J=5.13 Hz, 2H), 3.36 (s, 3H), 2.97-3.06 (m, 2H), 2.65-2.71 (m, 2H), 2.51 (s, 3H), 1.83-1.95 (m, 2H), 1.71-1.78 (m, 2H), 1.28-1.31 (m, 6H), 1.07 (s, 6H).

### Example 489, 2-(6,7-dihydro-4H-pyrazolo[5,1-c][1,4]oxazin-3-yl)-N-(5-((2-(3,3-dimethylazetidin-1-yl)ethyl)carbamoyl)-2-methylpyridin-3-yl)pyrazolo[5,1-b]thiazole-7-carboxamide

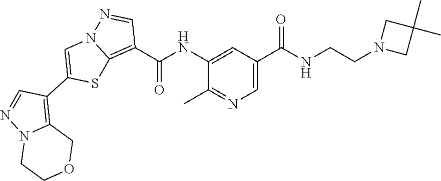 [SMILES: Cc1ncc(C(=O)NCCN2CC(C)(C)C2)cc1NC(=O)c1cnn2cc(-c3cnn4c3COCC4)sc12]

To a solution of 5-(2-(6,7-dihydro-4H-pyrazolo[5,1-c][1,4]oxazin-3-yl)pyrazolo[5,1-b]thiazole-7-carboxamido)-6-methylnicotinic acid (250 mg, 0.32 mmol), HATU (240 mg, 0.63 mmol) and N,N-diisopropylethylamine (0.25 mL, 1.4 mmol) in DMF (8 mL) was added 2-(3,3-dimethylazetidin-1-yl)ethanamine (110 mg, 0.88 mmol). The resulting mixture was stirred at room temperature for 1 hr and then concentrated under vacuum to give the crude product, which was purified by preparative high-performance liquid chromatography over column: Xtimate C18 150*40 mm*5 um to give the title compound 2-(6,7-dihydro-4H-pyrazolo[5,1-c][1,4]oxazin-3-yl)-N-(5-((2-(3,3-dimethylazetidin-1-yl)ethyl)carbamoyl)-2-methylpyridin-3-yl)pyrazolo[5,1-b]thiazole-7-carboxamide (36.5 mg, 21%) as a white solid. LCMS (ESI): mass calcd. for C₂₆H₃₀N₈O₃S, 534.2; m/z found, 535.2 [M+H]⁺. ¹H NMR (400 MHz, METHANOL-d₄) δ 8.80 (s, 1H), 8.43 (s, 1H), 8.34 (s, 1H), 8.11 (s, 1H), 7.84 (s, 1H), 5.07 (s, 2H), 4.24 (br d, J=4.6 Hz, 2H), 4.19 (br d, J=4.9 Hz, 2H), 3.49 (br t, J=6.1 Hz, 2H), 3.40 (s, 4H), 2.96 (br t, J=5.8 Hz, 2H), 2.63 (s, 3H), 1.30 (s, 6H).

### Example 490, 2-(2-(dimethylamino)pyridin-4-yl)-N-(5-((2-(2,2-dimethylpyrrolidin-1-yl)ethyl)carbamoyl)-2-methylpyridin-3-yl)pyrazolo[5,1-b]thiazole-7-carboxamide

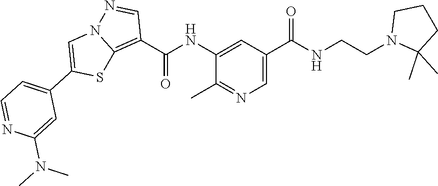 [SMILES: Cc1ncc(C(=O)NCCN2CCCC2(C)C)cc1NC(=O)c1cnn2cc(-c3ccnc(N(C)C)c3)sc12]

### Step a: N,N-dimethyl-4-(4,4,5,5-tetramethyl-1,3,2-dioxaborolan-2-yl)pyridin-2-amine

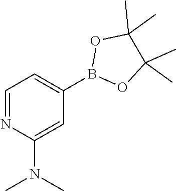 [SMILES: CN(C)c1cc(B2OC(C)(C)C(C)(C)O2)ccn1]

To a solution of 4-bromo-N,N-dimethylpyridin-2-amine (1 g, 5.0 mmol) in dioxane (35 mL) was added 4,4,4′,4′,5,5,5′,5′-octamethyl-2,2′-bi(1,3,2-dioxaborolane) (1.4 g, 5.5 mmol) and potassium acetate (0.75 g, 7.6 mmol), then Pd₂(dba)₃ (0.5 g, 0.55 mmol) and PCy₃ (280 mg, 1.0 mmol) was added. The resulting mixture was heated at 90° C. and stirred for 12 hours under N₂. The mixture was concentrated under vacuum to afford N,N-dimethyl-4-(4,4,5,5-tetramethyl-1,3,2-dioxaborolan-2-yl)pyridin-2-amine (1.2 g, 97%) as a black solid. The black solid was used for next step without purification.

### Step b: methyl 5-(2-(2-(dimethylamino)pyridin-4-yl)pyrazolo[5,1-b]thiazole-7-carboxamido)-6-methylnicotinate

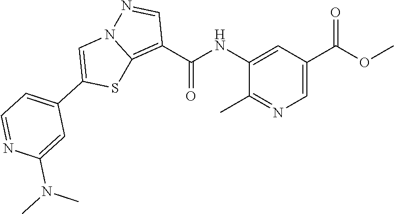 [SMILES: COC(=O)c1cnc(C)c(NC(=O)c2cnn3cc(-c4ccnc(N(C)C)c4)sc23)c1]

To the mixture of methyl 5-(2-bromopyrazolo[5,1-b]thiazole-7-carboxamido)-6-methylnicotinate (300 mg, 0.76 mmol) in 1,4-dioxane/H₂O=4:1 (20 mL) was added N,N-dimethyl-4-(4,4,5,5-tetramethyl-1,3,2-dioxaborolan-2-yl)pyridin-2-amine (200 mg, 0.80 mmol) and Cs₂CO₃ (650 mg, 2.0 mmol), then Pd(dppf)Cl₂·CH₂Cl₂ (150 mg, 0.18 mmol) was added. The resulting mixture was heated at 100° C. and stirred for 12 hours under N₂. The mixture was cooled to room temperature and filtered. The filter cake was triturated with EtOAc/DCM/H₂O/MeOH=10/10/5/1 (20 mL) at room temperature for 30 min, then mixture was filtered. The solid was washed with 10 mL EtOAc, collected and dried to dryness in vacuo to give 5-(2-(2-(dimethylamino)pyridin-4-yl)pyrazolo[5,1-b]thiazole-7-carboxamido)-6-methylnicotinate (730 mg, 78%) as a brown solid. LCMS (ESI): mass calcd. for C₂₁H₂₀N₆O₃S, 436.5; m/z found, 437.1 [M+H]⁺.

### Step c: 5-(2-(2-(dimethylamino)pyridin-4-yl)pyrazolo[5,1-b]thiazole-7-carboxamido)-6-methylnicotinic acid

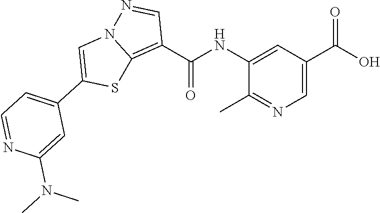 [SMILES: Cc1ncc(C(=O)O)cc1NC(=O)c1cnn2cc(-c3ccnc(N(C)C)c3)sc12]

To a solution of methyl 5-(2-(2-(dimethylamino)pyridin-4-yl)pyrazolo[5,1-b]thiazole-7-carboxamido)-6-methylnicotinate (700 mg, 0.57 mmol) in THF/MeOH=1/1 (30 mL) was added lithium hydroxide hydrate (140 mg, 3.3 mmol) in H₂O (100 mL) and the reaction was stirred at 20° C. for 1 hour. The reaction mixture was poured into 50 mL of water carefully and the aqueous phase was washed with DCM (100 mL×3) and acidified with 1N HCl to pH=3. The resulting precipitate was collected by filtration. The filter cake was rinsed with 10 mL of H₂O. The solid was collected, dried in vacuo to give 5-(2-(2-(dimethylamino)pyridin-4-yl)pyrazolo[5,1-b]thiazole-7-carboxamido)-6-methylnicotinic acid (300 mg, 90%) as a yellow solid. LCMS (ESI): mass calcd. for C₂₀H₁₈N₆O₃S, 422.1 m/z found, 423.1 [M+H]⁺.

### Step d: 2-(2-(dimethylamino)pyridin-4-yl)-N-(5-((2-(2,2-dimethylpyrrolidin-1-yl)ethyl)carbamoyl)-2-methylpyridin-3-yl)pyrazolo[5,1-b]thiazole-7-carboxamide

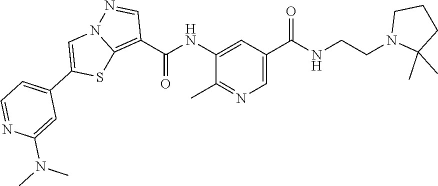 [SMILES: Cc1ncc(C(=O)NCCN2CCCC2(C)C)cc1NC(=O)c1cnn2cc(-c3ccnc(N(C)C)c3)sc12]

To the mixture of 5-(2-(2-(dimethylamino)pyridin-4-yl)pyrazolo[5,1-b]thiazole-7-carboxamido)-6-methylnicotinic acid (270 mg, 0.46 mmol), 2-(2,2-dimethylpyrrolidin-1-yl)ethanamine (110 mg, 0.76 mmol) and DIEA (0.4 mL, 2.420 mmol) in DMF (4 mL) was added HATU (240 mg, 0.63 mmol). The resulting mixture was heated at room temperature and stirred for 2 hours. Then the reaction was concentrated under vacuum to give the crude product, which was purified by preparative high-performance liquid chromatography over column: Phenomenex C18 80*40 mm*3 um to give the title compound 2-(2-(dimethylamino)pyridin-4-yl)-N-(5-((2-(2,2-dimethylpyrrolidin-1-yl)ethyl)carbamoyl)-2-methylpyridin-3-yl)pyrazolo[5,1-b]thiazole-7-carboxamide (33 mg, 18%) as a white solid. LCMS (ESI): mass calcd. for C₂₈H₃₄N₈O₂S, 546.7; m/z found, 547.3 [M+H]⁺. ¹H NMR (400 MHz, METHANOL-d₄) δ 8.80 (d, J=2.03 Hz, 1H), 8.77 (s, 1H), 8.50 (s, 1H), 8.34 (d, J=2.03 Hz, 1H), 8.13 (d, J=5.48 Hz, 1H), 6.83-6.94 (m, 2H), 3.56 (br t, J=6.91 Hz, 2H), 3.16 (s, 6H), 2.98 (br s, 2H), 2.74 (br s, 2H), 2.63 (s, 3H), 1.88 (dt, J=15.11, 7.64 Hz, 2H), 1.68-1.78 (m, 2H), 1.09 (s, 6H).

### Example 491, N-(5-((2-(cyclobutylamino)ethyl)carbamoyl)-2-methylpyridin-3-yl)-2-(5,6-dihydro-4H-pyrrolo[1,2-b]pyrazol-3-yl)pyrazolo[5,1-b]thiazole-7-carboxamide

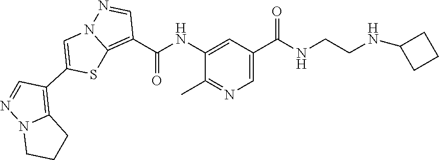 [SMILES: Cc1ncc(C(=O)NCCNC2CCC2)cc1NC(=O)c1cnn2cc(-c3cnn4c3CCC4)sc12]

### Step a: 2-(cyclobutylamino)acetonitrile

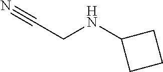

To a solution of cyclobutanamine (1 g, 14 mmol) in MeCN (20 mL) was added 2-bromoacetonitrile (0.97 mL, 13.9 mmol) and K₂CO₃ (6.4 g, 46 mmol) at 25° C. and the mixture was stirred for 2 hours. The mixture was collected and concentrated under vacuum to give a yellow oil. The yellow oil was purified by flash column chromatography over a 4 g silica gel (gradient: petroleum ether:ethyl acetate from 100:0 to 50:50), The filtrate was then concentrated to dryness under reduced pressure to afford the crude product 2-(cyclobutylamino)acetonitrile (1.42 g, 91%) as a yellow oil. LCMS (ESI): mass calcd. for C₆H₁₀N₂, 110.1; m/z found. 215.3 [M+H]⁺. ¹H NMR (400 MHz, CHLOROFORM-d) δ 3.55-3.53 (m, 2H), 3.48-3.40 (m, 1H), 2.34-2.25 (m, 2H), 1.83-1.71 (m, 4H).

### Step b: tert-butyl (cyanomethyl)(cyclobutyl)carbamate

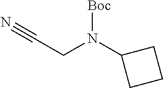 [SMILES: CC(C)(C)OC(=O)N(CC#N)C1CCC1]

A mixture of 2-(cyclobutylamino)acetonitrile (400 mg, 3.631 mmol) and di-tert-butyl dicarbonate (880 mg, 4.0 mmol) in MeOH (40 mL). The reaction mixture was stirred at room temperature overnight. The mixture was collected and concentrated under vacuum to give a yellow oil. The yellow oil was purified by flash column chromatography over a 4 g silica gel (gradient: petroleum ether:ethyl acetate from 100:0 to 80:20). The filtrate was then concentrated to dryness under reduced pressure to afford the crude product tert-butyl (cyanomethyl)(cyclobutyl)carbamate (600 mg, 78%) as a yellow oil. LCMS (ESI): mass calcd. for C₁₁H₁₈N₂O₂, 210.2; m/z found. 211.3 [M+H]⁺. ¹H NMR (400 MHz, CHLOROFORM-d) δ 4.15 (br s, 3H), 2.28-2.22 (m, 2H), 2.18-2.10 (m, 2H), 1.83-1.57 (m, 2H), 1.51 (s, 9H).

### Step c: tert-butyl (2-aminoethyl)(cyclobutyl)carbamate

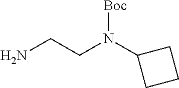 [SMILES: CC(C)(C)OC(=O)N(CCN)C1CCC1]

To a solution of tert-butyl (cyanomethyl)(cyclobutyl)carbamate (590 mg, 2.8 mmol) in THE (100 mL) under N₂ atmosphere. The reaction mixture was cooled to 0° C. via an ice water bath and once cooled for −10 mins, LiAlH₄ (148 mg, 3.9 mmol) was added portion wise (CAUTION: Evolved H₂ gas) at 0° C. The reaction was stirred at room temperature for 1 h. The filtrate was then concentrated to dryness under reduced pressure to afford the crude product tert-butyl (2-aminoethyl)(cyclobutyl)carbamate (350 mg, 58%) as a yellow oil. LCMS (ESI): mass calcd. for C₁₁H₂₂N₂O₂, 214.3; m/z found. 215.3 [M+H]⁺. ¹H NMR (400 MHz, METHANOL-d₄) δ 4.42-4.28 (m, 1H), 3.60-3.54 (m, 2H), 3.43-3.36 (m, 2H), 3.35-3.32 (m, 2H), 2.40-2.13 (m, 4H), 2.12-1.99 (m, 3H), 1.77-1.62 (m, 3H), 1.53-1.41 (m, 1H), 1.44 (s, 1H), 1.26 (s, 1H).

### Step d: tert-butyl cyclobutyl(2-(5-(2-(5,6-dihydro-4H-pyrrolo[1,2-b]pyrazol-3-yl)pyrazolo[5,1-b]thiazole-7-carboxamido)-6-methylnicotinamido)ethyl)carbamate

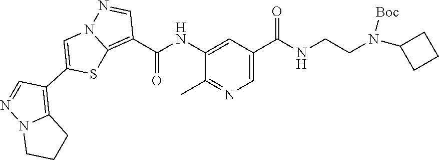 [SMILES: Cc1ncc(C(=O)NCCN(C(=O)OC(C)(C)C)C2CCC2)cc1NC(=O)c1cnn2cc(-c3cnn4c3CCC4)sc12]

To a solution of 5-(2-(5,6-dihydro-4H-pyrrolo[1,2-b]pyrazol-3-yl)pyrazolo[5,1-b]thiazole-7-carboxamido)-6-methylnicotinic acid (300 mg, 0.7 mmol), tert-butyl (2-aminoethyl)(cyclobutyl)carbamate (373 mg, 1.7 mmol) and DIEA (0.5 mL, 3.0 mmol) in DMF (8 mL) was added HATU (242 mg, 0.6 mmol). The resulting mixture was heated at room temperature for 2 hours. The mixture was poured into water and filtered, The filtrate was then concentrated to dryness under reduced pressure to afford the crude product tert-butyl cyclobutyl(2-(5-(2-(5,6-dihydro-4H-pyrrolo[1,2-b]pyrazol-3-yl)pyrazolo[5,1-b]thiazole-7-carboxamido)-6-methylnicotinamido) ethyl)carbamate (120 mg, 17%) as a white solid. LCMS (ESI): mass calcd. for C₃₀H₃₆N₈O₄S, 604.7; m/z found, 605.4 [M+H]⁺.

### Step e: N-(5-((2-(cyclobutylamino)ethyl)carbamoyl)-2-methylpyridin-3-yl)-2-(5,6-dihydro-4H-pyrrolo[1,2-b]pyrazol-3-yl)pyrazolo[5,1-b]thiazole-7-carboxamide

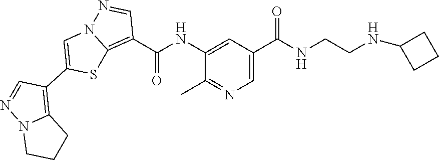 [SMILES: Cc1ncc(C(=O)NCCNC2CCC2)cc1NC(=O)c1cnn2cc(-c3cnn4c3CCC4)sc12]

To a solution of tert-butyl cyclobutyl(2-(5-(2-(5,6-dihydro-4H-pyrrolo[1,2-b]pyrazol-3-yl)pyrazolo[5,1-b]thiazole-7-carboxamido)-6-methylnicotinamido)ethyl)carbamate (100 g, 0.1 mmol) in DCM/MeOH=1:1 (6 mL) was added HCl/dioxane (1 mL, 4 mmol, 4M), The reaction mixture was stirred at room temperature for 2 h which was purified by preparative high-performance liquid chromatography over column: Xtimate C18 150*40 mm*5 um to give the title compound N-(5-((2-(cyclobutylamino)ethyl)carbamoyl)-2-methylpyridin-3-yl)-2-(5,6-dihydro-4H-pyrrolo[1,2-b]pyrazol-3-yl)pyrazolo[5,1-b]thiazole-7-carboxamide (17.3 mg, 290%) as a yellow solid. LCMS (ESI): mass calcd. for C₂₅H₂₈N₈O₂S, 550.6; m/z found, 505.3 [M+H]⁺. ¹H NMR (400 MHz, METHANOL-d₄) 8.83 (s, 1H), 8.51 (br s, 1H), 8.44 (s, 1H), 8.38 (s, 1H), 8.20 (s, 1H), 7.85 (s, 1H), 4.21 (t, J=7.3 Hz, 2H), 3.84-3.75 (m, 1H), 3.71 (br t, J=5.8 Hz, 2H), 3.13 (br t, J=6.1 Hz, 4H), 2.77 (quin, J=7.2 Hz, 2H), 2.65 (s, 3H), 2.36 (br d, J=6.8 Hz, 2H), 2.25-2.14 (m, 2H), 2.00-1.87 (in, 2H).

### Example 492, 2-(1-methyl-1H-pyrazol-4-yl)-N-(2-methyl-5-(3-(1-methylpyrrolidin-2-yl)propanamido)pyridin-3-yl)pyrazolo[5,1-b]thiazole-7-carboxamide

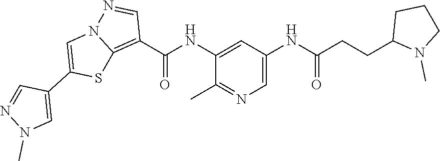 [SMILES: Cc1ncc(NC(=O)CCC2CCCN2C)cc1NC(=O)c1cnn2cc(-c3cnn(C)c3)sc12]

### Step a: tert-buty 2-(3-((6-methyl-5-(2-(1-methyl-1H-pyrazol-4-yl)pyrazolo[5,1-b]thiazole-7-carboxamido)pyridin-3-yl)amino)-3-oxopropyl)pyrrolidine-1-carboxylate

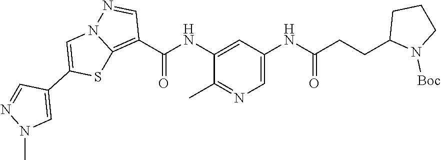 [SMILES: Cc1ncc(NC(=O)CCC2CCCN2C(=O)OC(C)(C)C)cc1NC(=O)c1cnn2cc(-c3cnn(C)c3)sc12]

To a solution of N-(5-amino-2-methylpyridin-3-yl)-2-(1-methyl-1H-pyrazol-4-yl)pyrazolo[5,1-b]thiazole-7-carboxamide (150 mg, 0.370 mmol) in DMF (7 mL) was added 3-(1-(tert-butoxycarbonyl)pyrrolidin-2-yl)propanoic acid (100 mg, 0.411 mmol) and 1-methyl-1H-imidazole (213 mg, 2.594 mmol), then N-(chloro(dimethylamino)methylene)-N-methylmethanaminium hexafluorophosphate(V) (208 mg, 0.741 mmol) was added at room temperature. The reaction mixture was stirred at room temperature for 2 hours. The reaction mixture was concentrated under vacuum to give a brown solid. The brown solid was subjected to column chromatography over 4 g silica gel (gradient: DCM:MeOH from 100:0 to 80:20). The pure fractions were collected and concentrated to dryness in vacuo to give tert-butyl 2-(3-((6-methyl-5-(2-(1-methyl-1H-pyrazol-4-yl)pyrazolo[5,1-b]thiazole-7-carboxamido)pyridin-3-yl)amino)-3-oxopropyl)pyrrolidine-1-carboxylate (320 mg, 104%) as a brown solid. LCMS (ESI): mass for C₂₈H₃₄N₈O₄S: 578.7; m/z found: 579.5 [M+H]⁺.

### Step b: 2-(1-methyl-1H-pyrazol-4-yl)-N-(2-methyl-5-(3-(pyrrolidin-2-yl)propanamido)pyridin-3-yl)pyrazolo[5,1-b]thiazole-7-carboxamide

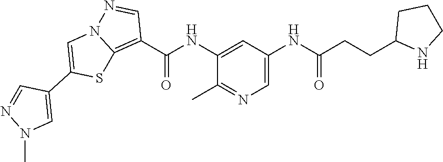 [SMILES: Cc1ncc(NC(=O)CCC2CCCN2)cc1NC(=O)c1cnn2cc(-c3cnn(C)c3)sc12]

To a solution of tert-butyl 2-(3-((6-methyl-5-(2-(1-methyl-1H-pyrazol-4-yl)pyrazolo[5,1-b]thiazole-7-carboxamido)pyridin-3-yl)amino)-3-oxopropyl)pyrrolidine-1-carboxylate (300 mg, 0.363 mmol) in DCM (10 mL) was added TFA (406 μL, 5.483 mmol) at room temperature. The reaction mixture was stirred at room temperature for 3 hours. The reaction mixture turned cloudy. The reaction mixture was concentrated in vacuo to give 2-(1-methyl-1H-pyrazol-4-yl)-N-(2-methyl-5-(3-(pyrrolidin-2-yl)propanamido)pyridin-3-yl)pyrazolo[5,1-b]thiazole-7-carboxamide (320 mg, 140%) as a brown solid. LCMS (ESI): mass for C₂₃H₂₆N₈O₂S: 478.57; m/z found: 479.1 [M+H]⁺.

### Step c: 2-(1-methyl-1H-pyrazol-4-yl)-N-(2-methyl-5-(3-(1-methylpyrrolidin-2-yl)propanamido)pyridin-3-yl)pyrazolo[5,1-b]thiazole-7-carboxamide

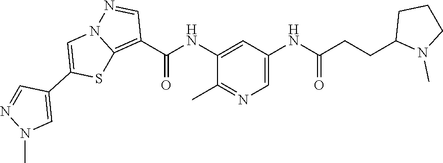 [SMILES: Cc1ncc(NC(=O)CCC2CCCN2C)cc1NC(=O)c1cnn2cc(-c3cnn(C)c3)sc12]

To a solution of paraformaldehyde (182 mg, 2.020 mmol) in MeOH (20 mL) was added 2-(1-methyl-1H-pyrazol-4-yl)-N-(2-methyl-5-(3-(pyrrolidin-2-yl)propanamido)pyridin-3-yl)pyrazolo[5,1-b]thiazole-7-carboxamide (300 mg, 0.547 mmol) and acetic acid (55 μL, 0.962 mmol) at 0° C. The mixture was stirred at room temperature for 0.5 hour. Then sodium cyanotrihydroborate (114 mg, 1.814 mmol) was added to the mixture. Then the mixture was stirred at 25° C. for 2 hours. The reaction mixture was concentrated under vacuum to give a brown solid. The brown solid was purified by Prep-HPLC over column: Xtimate C18 150*40 mm*5 um. Then purified by SFC over column: DAICEL CHIRALPAK AD (250 mm*30 mm, 10 um) The pure fractions were collected and the solvent was evaporated under vacuum, lyophilized to dryness to give 2-(1-methyl-1H-pyrazol-4-yl)-N-(2-methyl-5-(3-(1-methylpyrrolidin-2-yl)propanamido)pyridin-3-yl)pyrazolo[5,1-b]thiazole-7-carboxamide (21 mg, 8%) as an off-white solid. LCMS (ESI): mass for C₂₄H₂₈N₈O₂S: 492.6; m/z found: 493.2 [M+H]⁺. ¹H NMR (400 MHz, METHANOL-d₄) δ 8.63-8.50 (m, 1H), 8.46-8.34 (m, 1H), 8.24 (br d, J=3.8 Hz, 2H), 8.09-7.99 (m, 1H), 7.92-7.76 (m, 1H), 4.07-3.84 (m, 3H), 3.26-3.15 (m, 1H), 2.51 (br s, 6H), 2.49-2.31 (m, 4H), 2.28-2.09 (m, 2H), 1.91-1.78 (m, 2H), 1.72-1.53 (m, 2H).

### Example 493. 2-(5,6-dihydro-4H-pyrrolo[1,2-b]pyrazol-3-yl)-N-(5-(2-(isobutylamino)acetamido)-2-methylpyridin-3-yl)pyrazolo[5,1-b]thiazole-7-carboxamide

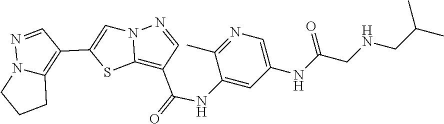 [SMILES: Cc1ncc(NC(=O)CNCC(C)C)cc1NC(=O)c1cnn2cc(-c3cnn4c3CCC4)sc12]

The title compound was prepared from 2-bromo-N-(5-(2-(isobutylamino)acetamido)-2-methylpyridin-3-yl)pyrazolo[5,1-b]thiazole-7-carboxamide according to the procedure of Example 372, substituting 3-(tetramethyl-1,3,2-dioxaborolan-2-yl)-4H,5H,6H-pyrrolo[1,2-b]pyrazole for 3-fluoro-5-(4,4,5,5-tetramethyl-1,3,2-dioxaborolan-2-yl)pyridin-2-amine (7 mg, 15%). LCMS (ESI): mass calcd. for C₂₄H₂₈N₈O₂S, 492.2; m/z found, 493.2 [M+H]⁺. ¹H NMR (DMSO-d6) δ: 9.87 (s, 1H), 8.43-8.63 (m, 3H), 8.18 (s, 1H), 7.87 (s, 1H), 4.13 (br t, J=7.3 Hz, 2H), 3.06 (br t, J=7.3 Hz, 2H), 2.64 (br dd, J=13.9, 7.1 Hz, 2H), 2.40 (s, 3H), 2.35 (br d, J=6.8 Hz, 2H), 1.57-1.83 (m, 1H), 0.89 (d, J=6.4 Hz, 6H).

### Example 494. N-(5-(2-(cyclobutylamino)acetamido)-2-methylpyridin-3-yl)-2-(6,7-dihydro-5H-pyrazolo[5,1-b][1,3]oxazin-3-yl)pyrazolo[5,1-b]thiazole-7-carboxamide

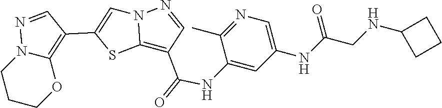 [SMILES: Cc1ncc(NC(=O)CNC2CCC2)cc1NC(=O)c1cnn2cc(-c3cnn4c3OCCC4)sc12]

The title compound was prepared from 2-bromo-N-(5-(2-(cyclobutylamino)acetamido)-2-methylpyridin-3-yl)pyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (3 mg, 5%). LCMS (ESI): mass calcd. for C₂₄H₂₆N₈O₃S, 506.6; m/z found, 507.3 [M+H]⁺. 1H NMR (METHANOL-d4) δ: 8.58 (s, 1H), 8.35 (s, 1H), 8.22 (d, J=1.5 Hz, 1H), 8.05 (s, 1H), 7.67 (s, 1H), 4.48-4.55 (m, 2H), 4.19 (t, J=6.1 Hz, 2H), 3.32-3.40 (m, 3H), 2.49 (s, 3H), 2.29-2.40 (m, 2H), 2.14-2.27 (m, 2H), 1.60-1.91 (m, 4H).

### Example 495. 2-(6,7-dihydro-5H-pyrazolo[5,1-b][1,3]oxazin-3-yl)-N-(2-methyl-5-(2-((1-methylcyclobutyl)amino)acetamido)pyridin-3-yl)pyrazolo[5,1-b]thiazole-7-carboxamide

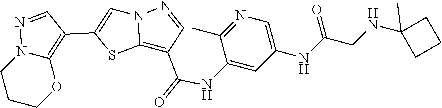 [SMILES: Cc1ncc(NC(=O)CNC2(C)CCC2)cc1NC(=O)c1cnn2cc(-c3cnn4c3OCCC4)sc12]

### Step a: 2-bromo-N-(2-methyl-5-(2-((1-methylcyclobutyl)amino)acetamido)pyridin-3-yl)pyrazolo[5,1-b]thiazole-7-carboxamide

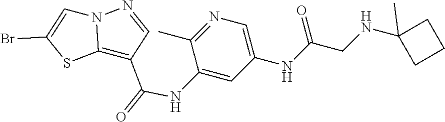 [SMILES: Cc1ncc(NC(=O)CNC2(C)CCC2)cc1NC(=O)c1cnn2cc(Br)sc12]

The title compound was prepared from 2-bromo-N-(5-(2-chloroacetamido)-2-methylpyridin-3-yl)pyrazolo[5,1-b]thiazole-7-carboxamide by the procedure of Example 364 step c replacing 3,3-dimethylazetidine hydrochloride with 1-methyl-cyclobutylamine (66 mg, 37%). LCMS (ESI): mass calcd. for C₁₉H₂₁BrN₆O₂S, 477.4; m/z found, 477.1/479.1 [M+H]⁺.

### Step b: 2-(6,7-dihydro-5H-pyrazolo[5,1-b][1,3]oxazin-3-yl)-N-(2-methyl-5-(2-((1-methylcyclobutyl)amino)acetamido)pyridin-3-yl)pyrazolo[5,1-b]thiazole-7-carboxamide

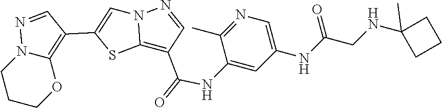 [SMILES: Cc1ncc(NC(=O)CNC2(C)CCC2)cc1NC(=O)c1cnn2cc(-c3cnn4c3OCCC4)sc12]

The title compound was prepared from 2-(6,7-dihydro-5H-pyrazolo[5,1-b][1,3]oxazin-3-yl)-N-(2-methyl-5-(2-((1-methylcyclobutyl)amino)acetamido)pyridin-3-yl)pyrazolo[5,1-b]thiazole-7-carboxamide according to the procedure of Example 372, substituting 3-(4,4,5,5-tetramethyl-1,3,2-dioxaborolan-2-yl)-6,7-dihydro-5H-pyrazolo[5,1-b][1,3]oxazine for 3-fluoro-5-(4,4,5,5-tetramethyl-1,3,2-dioxaborolan-2-yl)pyridin-2-amine (2 mg, 5%). LCMS (ESI): mass calcd. for C₂₃H₂₆N₈O₃S, 494.6; m/z found, 495.2 [M+H]⁺.

### Example 496. (S)-(1-methylpyrrolidin-2-yl)methyl (5-(2-(1,3-dimethyl-1H-pyrazol-4-yl)pyrazolo[5,1-b]thiazole-7-carboxamido)-6-methylpyridin-3-yl)carbamate

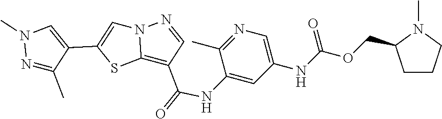 [SMILES: Cc1ncc(NC(=O)OC[C@@H]2CCCN2C)cc1NC(=O)c1cnn2cc(-c3cn(C)nc3C)sc12]

A 20 mL vial, equipped with a stir bar, was charged with N-(5-amino-2-methylpyridin-3-yl)-2-(1,3-dimethyl-1H-pyrazol-4-yl)pyrazolo[5,1-b]thiazole-7-carboxamide (97.5 mg, 0.241 mmol), THF (3.0 mL), N,N-diisopropylethylamine (0.20 mL, 1.1 mmol), and 1,1′-carbonyldiimidazole (68 mg, 0.42 mmol). After 24 h at 21° C., additional 1,1′-carbonyldiimidazole (105 mg, 0.646 mmol), N,N-diisopropylethylamine (0.10 mL, 0.57 mmol), and THE (1.0 mL) were placed in the reaction pot. After approximately 23 h at 21° C., solvent was removed under reduced pressure. The mixture was dissolved in DMF (3.0 mL) and transferred to a 2-5 mL microwave vial. N-methyl-L-prolinol (0.30 mL, 2.6 mmol) was added. A nitrogen atmosphere was established. The vial was sealed with a cap, and the mixture was irradiated in a Biotage Initiator+microwave reactor at 80° C. for 1 h. Solvent was removed under reduced pressure. The residue was purified by preparative high-performance liquid chromatography over column: Waters XBridge BEH C18, 5 μm, 19 mm×150 mm to provide (S)-(1-methylpyrrolidin-2-yl)methyl (5-(2-(1,3-dimethyl-1H-pyrazol-4-yl)pyrazolo[5,1-b]thiazole-7-carboxamido)-6-methylpyridin-3-yl)carbamate as a white solid (36 mg, 29%). LCMS (ESI): mass calcd for C₂₄H₂₈N₈O₃S, 508.2; m/z found, 509.2 [M+H]+. ¹H NMR (DMSO-d6, 400 MHz) δ 9.86 (s, 2H), 8.51 (s, 1H), 8.4-8.5 (m, 2H), 8.08 (s, 1H), 7.98 (d, 1H, J=1.2 Hz), 4.0-4.2 (m, 2H), 3.80 (s, 3H), 2.9-3.1 (m, 1H), 2.2-2.5 (m, 9H), 2.1-2.3 (f, 1H), 1.8-2.0 (m, 1H), 1.5-1.8 (ct, 3H).

### Biological Assays

PDGFRβ HTRF Assay

### I. Materials

Reagents

| Product | Vendor | Catalog# | Comments |
|---|---|---|---|
| 5x Kinase | Invitrogen by | PV6135 | Kinase Buffer A is supplied as a 5X |
| Buffer A | Thermo | | concentrated stock. For example, |
| | Fisher | | prepare a 1X solution by adding 4 mL |
| | Scientific | | of the 5X solution to 16 mL of distilled |
| | | | H2O. The 1X kinase reaction buffer is |
| | | | stable at room temperature. 1X Kinase |
| | | | Buffer A consists of 50 mM HEPES |
| | | | pH 7.5, 10 mM MgCl₂, 1 mM EGTA, |
| | | | 0.01% Brij-35. 100 mL per vial stored |
| | | | at room temperature |
| Molecular | Corning | 46-000-CM | 1 L vial stored at room temperature; |
| Biology Grade | | | LC-MS Grade |
| water | | | |
| DTT (DL | Sigma | D5545-1 G | 1 G vial was received as a solid and |
| Dithiothreitol) | | | dissolved with 3241 uL water to |
| | | | prepare a 2M stock; aliquot DTT and |
| | | | store at −20° C. storage; discard aliquot |
| | | | at EOB; MW = 154.25 g/mol |
| Albumin from | Sigma | A2512 | Received 250 mg and dissolved to a |
| Chicken Egg | | | 10% solution in water (prepare fresh on |
| White | | | experimantal day and discard at EOB); |
| | | | stored at 4 degrees; Lot#SLBN4912; |
| | | | MW = 44287 |
| His6-TEV- | Prepared at | | PDGFRb_08Prep02; 6.44 mg/mL or |
| PDGFRβ (557- | Accelagen | | 116 μM Stock; |
| 1016) | | | MW = 55455.3 g/mol; Storage Buffer |
| | | | contains 50 mM Tris-HCl, pH 8.0, 200 |
| | | | mM NaCl, 10% glycerol, 1 mM DTT; |
| | | | protein aliquots stored at −80° C. storage |
| DrySolv | Millipore | MX1457-7 | Stored at RT |
| Methylsufoxide | | | |
| A 384-well | PerkinElmer | 6008289 | Stored at RT |
| assay plate, | | | |
| ProxiPlate | | | |
| Plus, shallow | | | |
| well, white | | | |
| SMART small | Thermo | 24073295 | |
| tube metal tip | Fisher | | |
| dispensing | Scientific | | |
| cassette | | | |
| HTRF | Cisbio | 62TK0PEC | 20,000 tests; 4° C. storage; Allow the |
| KinEASE-TK | | | contents of the Cisbio kit to warm up to |
| kit | | | room temperature before use |
| Adenosine 5′- | Sigma | A7699 | Prepare a 100 mM solution in water; |
| triphosphate | | | BioXtra, |
| (ATP) | | | ≥99% (HPLC); −20° C. storage |
| disodium salt | | | |
| hydrate | | | |

Instrumentation: a. Compound liquid handling: LabCyte Echo; b. Reagent liquid handling: Thermo Scientific Multidrop Combi; c. BMG PHERAStar multilabel plate reader.

Protein Reagent: His6-TEV-PDGFRβ Protein Prep prepared at Accelagen.

### II. Methods and Procedures

Stock Solutions:

Assay buffer stock solution, contains 50 mM Hepes, 10 mM MgCl₂, 1 mM EGTA, and 0.01% Brij-35, 0.01% ovalbumin, 2 mM DTT at pH 7.5, in molecular biology grade water. Store at room temperature.

DTT, 2 M in molecular biology grade water, store at −20° C. in aliquots.

Ovalbumin, 10% or 100 mg/mL, prepare fresh on experimental day.

PDGFRβ, 116 μM (PDGFRb_08 Prep 02), produced at Accelagen. Store at −80° C. in aliquots.

TK-biotin peptide, 0.5 μM in molecular biology grade water, store at −20° C. in aliquots.

ATP, 100 mM in molecular biology grade water, store at −20° C. in aliquots.

HTRF KinEASE-TK kit: Allow the contents of the Cisbio kit to warm up to room temperature before use. This kit contains HTRF detection buffer, TK-Antibody labeled with Eu³⁺-cryptate, TK-substrate biotin and Streptavidin-XL665.

TK Substrate-Biotin, reconstitute 500 μg lyopholized with 574 μL molecular biology grade water to prepare a 500 μM stock; After use, aliquot the rest and store at −20° C.

TK Antibody-Cryptate, reconstitute lyophilized with 1 mL of molecular biology grade water (100× solution) then add 99 mL detection buffer to prepare a ready to use TK-antibody-cryptate solution; the concentration of the TK-antibody-cryptate reagent is not known. After use, aliquot the rest and store at −20° C.

Streptavidin-XL665, reconstitute 3 mg lyophilized with 3 mL molecular biology grade water to prepare a 1 mg/mL or 16.67 μM stock; MW=60 kDa; After use, aliquot the rest and store at −20° C.

Freshly Prepared Solutions:

Assay buffer. Dilute 5× Kinase buffer 5-fold with molecular biology grade water and add DTT to 2 mM and ovalbumin to 0.1 mg/mL (or 0.01%).

2× protein solution. Make a working solution of 100 μM PDGFRβ in assay buffer. Keep on ice until use to maintain enzyme stability.

2× substrate solution. Make a working solution of 1.6 mM ATP and 1 μM TK-substrate biotin peptide in assay buffer.

3× quench/detection solution. Make a working solution of 0.1875 μM SA-XL665 and the TK-antibody cryptate diluted by 12 of total quench/detection volume in assay buffer.

Keep final streptavidin/biotin ratio at 1 to 8.

Example of 3× quench/detection solution preparation: 8 mL total volume.

1× assay buffer—3910 μL

TK antibody-cryptate in detection buffer—4000 μL

0.1875 μM SA-XL665-90 uL

The kinase reaction is stopped by the addition of the detection reagents which contain EDTA (detection step).

Assay Procedure:

Assay in white ProxiPlate 384-well

Step 1. Dispensing inhibitors/DMSO and low control: Using the ECHO 555 acoustic dispenser, spot desired compound serial dilutions in DMSO, NEAT DMSO to represent the uninhibited enzyme control, and 10 μM final [imatinib] to the represent the 100% inhibited enzyme control

Step 2. PDGFRβ E+I pre-incubation: Add 2 μL 2× protein solution to columns 1-24 using the Multidrop Combi. Centrifuge at 1000 rpm for 1 min. Incubate 30 min at RT

Step 2. Enzymatic reaction: Add 2 μL substrate solution to columns 1-24 to initiate the reaction using the Multidrop Combi; cover/seal the assay plate to reduce evaporation. Centrifuge at 1000 rpm for 1 min. Incubate at room temperature for 3 hours.

Final concentrations of components in PDGFRβ cascade assay:
50 mM Hepes, pH 7.5
10 mM MgCl2
0.01% Brij-35
1 mM EGTA
2 mM DTT
0.01% Ovalbumin
50 μM inactive PDGFRβ
0.5 μM TK-substrate biotin peptide
62.5 nM SA-XL-665
TK antibody-Eu3+-cryptate (diluted by ⅓ final from stock)
800 μM ATP
≤1% DMSO

Step 3. Quench/Detection: Add 2 μl 3× quench/detection solution to columns 1-24 using the Multidrop Combi; cover/seal the plate. Centrifuge 1 min 1000 rpm. Incubate at RT for 60 min. Read the plate in PHERAstar (or similar instrument) on HTRF setting at excitation 337 nm—dual emission—665/620 nm ratio.

### III. Calculations and Formulas

HTRF ratio values calculated by the instrument (Ratio is acceptor counts/donor counts*10,000) is exported from the plate reader and used in data analysis. The exported data will be used to calculate 1) compound activity and 2) assay statistics. Compound activity is represented by % Inhibition when testing a single dose of a compound or IC₅₀ when testing a dose response of a compound. Assay statistics can include Robust Z′ and Signal to Background.

% Inhibition Calculation: Percent inhibition will be calculated for sample wells based on the equation:

$$\% Inhibition = [ \frac{( x - cr )}{( sr - cr )} ] * 100 )$$

Where, x: sample activity; cr: central reference is calculated based on wells containing all assay components and no compound (DMSO only); sr: scale reference is calculated based on wells inhibited with 10 μM Imatinib (these wells will contain the enzyme and substrate solutions)

IC₅₀ Calculation: For IC₅₀ determination, full 11-point dose response data will be processed using the following equation:

$$Y = {S}_{0} + \frac{{S}_{Inf} - {S}_{0}}{1 + {( \frac{IC 50}{{10}^{c}} )}^{n}}$$

Where SO=Activity level at zero concentration of test compound; SInf=Activity level at infinite concentration; IC50: Concentration at which activity reaches 50% of maximum level; c=Concentration in logarithmic units corresponding to the values on the x-axis of the dose-response curve plot; Hill coefficient n=Measure of the slope at IC50. See Table 2, below.

Registered Parameters (when applicable): % Activity, IC₅₀, nHill Slope, S_{inf}, S₀, and Comments

Robust Z′ Calculation: Robust Z prime (RZ′) value will be calculated as defined by the following equation:

$${RZ}^{′} = 1 - \frac{3 * ( {RSD}_{cr} + {RSD}_{sr} )}{\vert {median}_{cr} - {median}_{sr} \vert}$$

Where, RSD: Robust standard deviation; cr: central reference is calculated based on wells containing all assay components and no compound (DMSO only); sr: scale reference is calculated based on wells inhibited with 10 μM Imatinib compound (these wells will contain the enzyme and substrate solutions)

Signal to Background, S/B, Calculation

$$\frac{S}{B} = \frac{median ( CR )}{median ( SR )}$$

Where, CR, Central Reference (no compound wells); SR, Scale Reference (inhibitor control wells).

PDGFRβ LanthaScreen Assay

### I. Materials

| Reagent | Manufacturer | Catalog # | Lot # |
|---|---|---|---|
| MilliQ water | In-house | N/A | |
| HEPES, 1M, pH 7.5 | Teknova | H1035 | |
| EGTA | Sigma | 03777-10 G | BCBW6026 |
| MgCl₂ | Sigma | M1028-100 mL | SLBP0426V |
| Brij-35 | EMD | 203728-50 mL | S159338 |
| DTT | Sigma | D5545-1 G | |
| Ovalbumin | Sigma | A2512-5 G | SLBT1992 |
| Tracer 222 | Invitrogen | PV6121 | 2066013B |
| DMSO | Sigma | D8418-100 ML | SHBG5286V |
| Tb-labeled inactive | In-house | N/A | |
| PDGFRβ | | | |
| 384 well plate | Greiner | 784075 | |
| Plate seal tape—iCycler | BioRad | 2239444 | |
| iQ optical tape | | | |

### II. Methods and Procedures

Stock Solutions:

Assay buffer stock contains 50 mM HEPES pH7.5, 10 mM MgCl₂, 0.01% Brij-35, 1 mM EGTA.

Tb-labeled inactive PDGFRβ. 3.6 μM in 50 mM HEPES, pH 7.4, 150 mM NaCl, 0.005% Tween-20 and 10% glycerol. Store at −80° C. in aliquots.

Tracer 222, 50 μM in DMSO, store at −20° C.

Freshly Prepared Solutions:

Assay buffer. Add DTT to 2 mM and ovalbumin to 0.1 mg/mL to Assay buffer stock.

Kinase-Tracer solution. Make a working solution of 0.2 nM Tb-labeled inactive PDGFRβ and 40 nM Tracer 222 in Assay buffer. Keep on ice until use.

Assay Procedure:

Step 1. Dispensing inhibitors: Using Echo, dispense 40 nL/well (or less) compound serial dilutions in DMSO onto the assay plate.

Step 2. Dispensing Kinase-Tracer solution: Add 4 μL/well Kinase-Tracer solution. Seal the plate with optically transparent plate seal. Centrifuge at 1000 rpm for 1 min.

Final concentrations of components in the assay:

[Tb-PDGFRβ]=0.2 nM;

[Tracer 222]=40 nM;

[DMSO]≤1%.

Step 3. Detection: Read TR-FRET signals after 18 hours incubation at room temperature.

### III. Calculations and Formulas

% Inhibition: % Inhibition=(NC−sample)/(NC−PC)*100 where NC is the mean of negative control (reactions without inhibitor), and PC is the mean of positive control (1 μM sunitinib).

IC₅₀ determination: Compounds are serially diluted 3-fold and tested in an 11-point dose response. IC₅₀ values are determined from a 4-parameter fit, using the following equation: Y=Bottom+(Top−Bottom)/(1+10((Log IC₅₀−X)*Hill slope)), where X=log₁₀ of the compound concentration; top can be defined by PC; bottom defined by NC. See Table 2, below.

PDGFRβ Cellular Assay

### I. Materials

| Material Description | Vendor | Catalog |
|---|---|---|
| A10 Cells | ATCC | CRL-1476 |
| Rat Embryonic Aortic Smooth Muscle | | |
| Cells (ASMC) | | |
| Dulbecco's Modified Eagle Medium (DMEM) | Gibco | 11965 |
| Fetal Bovine Serum (FBS) | Gibco | 16000 |
| Penicillin-Streptomycin (10,000 U/mL) (P/S) | Gibco | 15140 |
| PBS without Ca2+ and without Mg2+ | Corning | 21-031-CM |
| 0.25% Trypsin EDTA | Gibco | 25200 |
| Platelet-derived Growth Factor (PDGF-BB); | R&D | 520-BB-050 |
| Rat | Systems | |
| PDGFRb P-Y751 Kit, 10,000 tests | CisBio | 64- |
| | | PDGPEH |
| Greiner cell culture plate, poly-D-lysine, | Greiner | 781945 |
| cell coat 384 Well Plates | | |
| Dimethyl Sulfoxide (DMSO) | Sigma | MX1457 |

### II. Methods and Procedures

Cell Culture and Preparation: Cells are cultured according to ATCC procedure (5) with the addition of the antibiotic penicillin-streptomycin. If working from frozen, cells should be thawed according to ATCC procedure. Depending on the cell density of frozen vial, cells will need time to recover from thaw. An 80% confluent T75 flask should be enough for one 384 well plate.

Stock Solutions: Rat PDGFBB. A 100 ug/mL stock is prepared by reconstituting 50 ug in 500 uL of 4 mM HCl and 0.1% BSA. It can be stored for a month at 4° C., or aliquoted out and frozen in the −20/−80° C. to avoid multiple free-thaw cycles.

Freshly Prepared Solutions:

1× Cisbio cell lysis buffer. The cell lysis buffer is diluted 4-fold with molecular grade water. The blocking agent is then diluted 25-fold in the diluted lysis buffer.

Antibody solutions. Equal amounts d2 and cryptate antibody are diluted 20-fold in detection buffer.

Rat PDGFBB. A working stock of 100 ng/mL is created from the stock solution in 10% FBS culture media.

Assay Steps

Step 1: Plating Cells: Media from the A10 cell flask is aspirated. The cells are rinsed with PBS and then trypsinized to disperse the cell layer. The cells are then pelleted are resuspended to 1.25e5 cells/mL. 40 uL of cells are then plated in 384 Greiner TC treated plates at a density of 5000 cells/well using the Combi. Plates are covered and placed in the incubator (37° C. 5% CO₂) overnight to allow cells to adhere.

Step 2: Compound Dispense: Approximately 18 hours after plating, dispense 40 nL compound onto cells using Echo. Column 12 is the neutral control DMSO, column 24 is the inhibitor control 10 mM Imatinib (10 uM final assay concentration). The plate is returned to the incubator for 3 hours.

Step 3: Activation by PDGFbb: 6 uL of the working stock of 100 ng/mL PDGFbb is dispensed using the Tempest to give a final assay concentration of 15 ng/mL (EC80). After 10 minutes the media is removed by flicking the plate.

Step 4: Cell lysis and antibody addition: 20 uL lysis buffer per well is added to the plate via Tempest. 5 uL antibody solution is added per well via the Tempest. The plate is placed on the shaker at 230 rpm for 1 hour at room temperature.

Step 5: Detection: The plate is read using the HTRF module on the BMG Pherastar. Data is analyzed using Genedata Screener.

### III. Calculations and Formulas

% Inhibition: % Inhibition=(NC−sample)/(NC−PC)*100 where NC is the mean of negative control (reactions without inhibitor), and PC is the mean of positive control (10 μM imatinib).

IC₅₀ determination: Compounds are serially diluted 3-fold and tested in an 11-point dose response. IC₅₀ values are determined from a 4-parameter fit, using the following equation: Y=Bottom+(Top−Bottom)/(1+10((Log IC₅₀−X)*Hill slope)), where X=log₁₀ of the compound concentration; top can be defined by PC; bottom defined by NC. See Table 2, below.

VEGFR ADP GLO Assay

### I. Materials

| Reagent | Manufacturer | Catalog # | Lot # |
|---|---|---|---|
| MilliQ water | In-house | N/A | |
| HEPES, 1M, | Teknova | H1035 | |
| pH 7.5 | | | |
| EGTA | Sigma | 03777-10 G | BCBW6026 |
| MgCl₂ | Sigma | M1028-100 mL | SLBP0426V |
| Brij-35 | EMD | 203728-50 mL | S159338 |
| DTT | Sigma | D5545-1 G | |
| Ovalbumin | Sigma | A2512-5 G | SLBT1992 |
| DMSO | Sigma | D8418-100 ML | SHBG5286V |
| VEGFR2, | Accelagen | N/A | EC_VEGFR2_03 |
| unphosphorylated | | | Prep01A, |
| | | | Accelagen |
| | | | 20190321 |
| Srctide | Anaspec | AS-64105 | |
| ADP Glo | Promega | V9102 | |
| TAK-593 | MedChemExpress | HY-15506 | 36686 |
| Pluronic F-127 | Millipore | 540025-50 ML | |
| 384-well assay | Perkin Elmer | 6008289 | |
| plate (white, | | | |
| non-treated | | | |
| Proxiplate) | | | |
| 1536-well assay | Corning | 9146BC | |
| plate, (black, | | | |
| non-treated) | | | |

### II. Methods and Procedures

Stock Solutions:

Assay buffer stock contains 50 mM HEPES pH7.5, 10 mM MgCl₂, 0.01% Brij-35, and 1 mM EGTA.

Unphosphorylated VEGFR2. 52.6 μM in 50 mM Tris-HCl, pH 8.0, 50 mM NaCl, 5% Glycerol, 0.5 mM TCEP. Store at −80° C. in aliquots.

10 mg/mL srctide solution, prepared in Assay buffer (Assay buffer stock with 2 mM DTT, 0.1% Pluronic F-127, and 0.1 mg/mL ovalbumin). Sonicate 10 mg/ml Srctide solution for 10 minutes and then vacuum filter.

Freshly Prepared Solutions

Assay buffer. Add DTT to 2 mM, Pluronic F-127 to 0.1% and ovalbumin to 0.1 mg/mL to Assay buffer stock.

2× kinase solution. Make a working solution of 10 nM unphosphorylated VEGFR2 in Assay buffer. Vacuum filter 2× kinase solution prior to running assay. Keep on ice until use.

2× substrate/ATP solution. Make a working solution of 2 mg/mL srctide and 2.4 mM ATP in Assay buffer. Keep on ice until use.

Assay Procedure:

Step 1. Dispensing inhibitors/controls: Using Echo, dispense 10 nL/well compound serial dilutions in DMSO to columns 1-22 (in 384-well plates) or columns 1-44 (in 1536-well plates). Dilution series=11 pt, 3-fold dilutions. The top compound concentration in the source plate is 4 mM. The top compound concentration in the assay plate is 10 uM. Using Echo, dispense 10 nl/well DMSO to column 23 (in 384-well plates) or columns 45-47 (in 1536-well plates). These wells will serve as negative control wells Using Echo, dispense 10 nl/well 400 uM TAK-593 in DMSO to column 24 (in 384-well plates) or column 48 (in 1536-well plates). The final concentration of TAK-593 in the assay should be 1 uM. These wells will serve as positive control wells.

Step 2. Pre-incubation of inhibitors with kinase: Add 2 μL/well 2× kinase solution. Centrifuge at 1000 rpm for 1 min. Incubate at room temperature for 30 min.

Step 3. Kinase cascade reaction: Add 2 μL/well 2× substrate/ATP solution to initiate kinase reactions. Centrifuge at 1000 rpm for 1 min. Incubate at room temperature for 180 min.

Final concentrations of components in the assay:

[VEGFR2]=5 nM;

[ATP]=1.2 mM;

[Srctide]=1 mg/mL;

[DMSO] G; 1%.

Step 4. Quench: Add 2 uL/well ADP Glo Reagent+0.05% CHAPS. Centrifuge at 1000 rpm for 1 min; Incubate at room temperature for one hour.

Step 5. Detection: Add 2 uL/well Kinase Detection Reagent+0.05% CHAPS. Centrifuge at 1000 rpm for 1 min; Incubate at room temperature for 1 hour; Read Luminescence on a plate reader.

### III. Calculations and Formulas

% Inhibition: % Inhibition=(NC−sample)/(NC−PC)*100 where NC is the mean of negative control (reactions without inhibitor), and PC is the mean of positive control (1 μM TAK-593).

IC₅₀ determination: Compounds are serially diluted 3-fold and tested in an 11-point dose response. IC₅₀ values are determined from a 4-parameter fit, using the following equation: Y=Bottom+(Top−Bottom)/(1+10^{(Log IC50-X)*Hill slope}) where X=log₁₀ of the compound concentration; top can be defined by PC; bottom defined by NC. See Table 2, below.

TABLE 2
Biology chemical and cellular activity

| | PDGFR | PDGFR | PDGFR | VEGFR |
|---|---|---|---|---|
| | HTRF | Lantha | Cellular | ADP GLO |
| Example # | IC₅₀ nM | IC₅₀ nM | IC₅₀ nM | IC₅₀ nM |
| 1 | 0.2 | 2.3 | 33.8 | >7,500 |
| 2 | 0.7 | 11.5 | 30.2 | >7,500 |
| 3 | 0.6 | 4.0 | 6.0 | 6,980 |
| 4 | 0.3 | 2.3 | 11.7 | >7,500 |
| 5 | 0.4 | 5.8 | 26.2 | >7,500 |
| 6 | 0.2 | 4.7 | 7.7 | >7,500 |
| 7 | 0.5 | 8.3 | 15.8 | >7,500 |
| 8 | 0.4 | 4.7 | 43.6 | >10,000 |
| 9 | 0.3 | 3.5 | 16.8 | >7,500 |
| 10 | 0.3 | 2.8 | 5.2 | >7,500 |
| 11 | 0.5 | 5.0 | 10.6 | >7,500 |
| 12 | 0.3 | 3.3 | 7.7 | >7,500 |
| 13 | 0.5 | 5.0 | 3.3 | >7,500 |
| 14 | 0.4 | 2.7 | 4.4 | >7,500 |
| 15 | 0.5 | 4.7 | 15.7 | >7,500 |
| 16 | 0.5 | 5.1 | 4.1 | >7,500 |
| 17 | 0.5 | 6.0 | 69.5 | >7,500 |
| 18 | 0.6 | 9.5 | 49.8 | >7,500 |
| 19 | 0.7 | 4.2 | 12.9 | >7,500 |
| 20 | 0.7 | 8.2 | 35.9 | >7,500 |
| 21 | 0.7 | 5.7 | 10.9 | >7,500 |
| 22 | 0.7 | 8.6 | 5.3 | >7,500 |
| 23 | 0.7 | 1.3 | 67.7 | >7,500 |
| 24 | 0.7 | 4.5 | 31.8 | >7,500 |
| 25 | 0.7 | 6.6 | 27.7 | >7,500 |
| 26 | 0.8 | 11.7 | 5.9 | >7,500 |
| 27 | 0.9 | 16.2 | 6.3 | >7,500 |
| 28 | 0.6 | 4.2 | 13.6 | >7,500 |
| 29 | 0.4 | 3.4 | 5.0 | >7,500 |
| 30 | 0.5 | 3.3 | 16.5 | >7,500 |
| 31 | 0.6 | 5.1 | 11.1 | >7,500 |
| 32 | 0.7 | 5.5 | 20.5 | >7,500 |
| 33 | 0.7 | 9.3 | 10.1 | >7,500 |
| 34 | 1.7 | 13.4 | 30.3 | >7,500 |
| 35 | 1.6 | 71.4 | 33.6 | >7,500 |
| 36 | 0.5 | 19.3 | 9.0 | >7,500 |
| 37 | 22.8 | 892 | 11.2 | >7,500 |
| 38 | 1.8 | 33.5 | 3,000 | >7,500 |
| 39 | 0.3 | 1.9 | 2.0 | 2,040 |
| 40 | 0.27 | 1.7 | 2.0 | 6,180 |
| 41 | 0.43 | 4.7 | 10 | >7,500 |
| 42 | 0.28 | 3.8 | 5.9 | >7,500 |
| 43 | 0.23 | 3.9 | 7.2 | >7,500 |
| 44 | 1.3 | 11.6 | 23.7 | >7,500 |
| 45 | 1.0 | 31.0 | 75.4 | >7,500 |
| 46 | 3.0 | 89.0 | 85.5 | >7,500 |
| 47 | 0.9 | 16.5 | 37.2 | >7,500 |
| 48 | 1.4 | 16.8 | 49.3 | >7,500 |
| 49 | 0.4 | 2.1 | 4.7 | >7,500 |
| 50 | 0.6 | 14.0 | 27.3 | >7,500 |
| 51 | 0.4 | 4.8 | 6.8 | >7,500 |
| 52 | 0.3 | 0.8 | 0.9 | >7,500 |
| 53 | 0.3 | 2.7 | 4.3 | >7,500 |
| 54 | 0.5 | 12.8 | 11.4 | >7,500 |
| 55 | 1.5 | 40.6 | 87.1 | >7,500 |
| 57 | 1.0 | 10.8 | 124.2 | >7,500 |
| 58 | 1.0 | 12.5 | 52.9 | >7,500 |
| 59 | 2.1 | 93.9 | >3000 | >7,500 |
| 60 | 1.0 | 18.3 | 50.4 | >7,500 |
| 61 | 0.8 | 12.3 | 43.6 | >7,500 |
| 62 | 1.4 | 17.6 | 110.9 | >7,500 |
| 63 | 2.9 | 42.9 | 223.4 | >7,500 |
| 64 | 0.4 | 6.3 | 8.6 | >7,500 |
| 65 | 0.7 | 16.5 | 6.8 | >7,500 |
| 66 | 0.3 | 2.6 | 4.6 | >7,500 |
| 67 | 1.0 | 5.7 | 8.3 | >7,500 |
| 68 | 0.8 | 8.1 | 19.1 | >7,500 |
| 69 | 0.5 | 4.7 | 12.1 | >7,500 |
| 70 | 0.2 | 2.9 | 6.4 | >7,500 |
| 71 | 2.9 | 47.9 | 90.9 | >7,500 |
| 72 | 5.7 | 112.1 | >3000 | >7,500 |
| 73 | 0.3 | 1.7 | 3.2 | >7,500 |
| 74 | 1.0 | 24.2 | 41.6 | >7,500 |
| 75 | 4.1 | 47.0 | 2748.5 | >7,500 |
| 76 | 0.9 | 8.9 | 36.9 | >7,500 |
| 77 | 1.8 | 15.9 | 78.9 | >7,500 |
| 78 | 0.9 | 5.7 | 12.1 | >7,500 |
| 79 | 0.8 | 10.9 | 12.6 | >7,500 |
| 80 | 1.4 | 19.3 | 37.8 | >7,500 |
| 81 | 2.8 | 76.8 | 79.8 | >7,500 |
| 82 | 1.6 | 24.3 | 12.5 | >7,500 |
| 83 | 2.3 | 35.0 | 44.1 | >7,500 |
| 84 | 0.9 | 10.9 | 40.2 | >7,500 |
| 85 | 1.7 | 13.4 | 30.3 | >7,500 |
| 86 | 1.3 | 7.3 | 18.4 | >7,500 |
| 87 | 0.4 | 3.2 | 1 | >7,500 |
| 88 | 0.4 | 4.2 | 141.2 | >7,500 |
| 89 | 0.6 | 12.7 | 26.2 | >7,500 |
| 90 | 0.5 | 10.6 | 19.9 | >2,500 |
| 91 | 0.5 | 13.2 | 36.4 | >7,500 |
| 92 | 0.5 | 7.2 | 121.7 | >7,500 |
| 93 | 0.9 | 29.1 | 66.0 | >7,500 |
| 94 | 0.5 | 14.2 | 142.7 | >7,500 |
| 95 | 0.4 | 5.1 | 17.9 | >7,500 |
| 96 | 0.6 | 28.4 | 17.0 | >7,500 |
| 97 | 0.4 | 6.4 | 8.4 | >7,500 |
| 98 | 0.3 | 2.9 | 4.7 | >7,500 |
| 100 | 0.5 | 7.5 | 33.4 | >7,500 |
| 102 | 1.3 | 1 | 21.8 | >7,500 |
| 103 | 0.8 | 8.6 | 38.8 | >7,500 |
| 104 | 0.4 | 9.8 | 9.2 | >7,500 |
| 106 | 1.3 | 67.0 | 461.2 | >7,500 |
| 107 | 13.2 | 581.8 | >3000 | >7,500 |
| 108 | 10.7 | 490.3 | >3000 | >2,500 |
| 109 | 38.9 | 1902.0 | >3000 | >7,500 |
| 110 | 0.6 | 5.7 | 35.4 | 664 |
| 111 | 1.1 | 36.4 | 294.1 | >7,500 |
| 112 | 0.5 | 12.6 | 159.4 | >7,500 |
| 113 | <0.1 | 1.4 | 11.5 | >7,500 |
| 114 | 0.4 | 4.6 | 27.6 | >7,500 |
| 115 | 0.4 | 4.7 | 13.3 | >7,500 |
| 116 | 0.5 | 12.7 | 74.7 | >7,500 |
| 117 | 0.4 | 2.3 | 5.6 | >7,500 |
| 118 | 0.3 | 7.1 | 55.5 | >7,500 |
| 119 | 0.3 | 2.0 | 4.9 | >7,500 |
| 120 | 0.4 | 4.5 | 9.3 | >7,500 |
| 121 | 0.4 | 13.1 | 80.4 | >7,500 |
| 122 | 0.3 | 2.8 | 19.4 | >7,500 |
| 123 | 0.3 | 2.7 | 7.8 | >7,500 |
| 124 | 0.6 | 19.6 | 133.8 | >7,500 |
| 125 | 0.3 | 5.3 | 47.6 | >7,500 |
| 126 | 0.3 | 10.3 | 80.1 | >7,500 |
| 127 | 0.6 | 6.0 | 16.2 | >7,500 |
| 128 | 0.9 | 18.3 | 46.8 | >7,500 |
| 129 | 2.9 | 87.1 | >3000 | >7,500 |
| 130 | 0.5 | 4.3 | | >7,500 |
| 131 | 1.7 | 9.1 | | >7,500 |
| 133 | 1.5 | 6.3 | | >7,500 |
| 134 | 2.9 | 13.9 | | >7,500 |
| 135 | 1.3 | 10.6 | | >7,500 |
| 136 | 1.4 | 1 | | >7,500 |
| 137 | 2.2 | 24.4 | | >2,500 |
| 138 | 3.7 | 39.6 | | >7,500 |
| 139 | 5.3 | 181.6 | 163.7 | >7,500 |
| 140 | 0.5 | 4.9 | 308.8 | >7,500 |
| 141 | 1.8 | 18.6 | >3000 | >7,500 |
| 142 | 0.5 | 4.2 | 71.7 | >7,500 |
| 143 | 0.6 | 5.3 | 997.7 | >7,500 |
| 144 | 0.3 | 3.6 | 106.2 | >7,500 |
| 145 | 0.2 | 1.4 | 407.0 | >833 |
| 146 | 0.6 | 4.3 | 858.0 | >7,500 |
| 147 | 0.8 | 6.9 | >2000 | >7,500 |
| 148 | 0.3 | 4.2 | 28.9 | >7,500 |
| 149 | 0.3 | 5.0 | 36.2 | >7,500 |
| 150 | 0.6 | 5.5 | >2000 | >7,500 |
| 151 | 0.3 | 2.8 | 239.7 | >7,500 |
| 152 | 0.4 | 12.4 | 66.2 | >7,500 |
| 153 | 0.4 | 18.0 | 105.6 | >7,500 |
| 154 | 0.3 | 2.4 | 8.7 | >7,500 |
| 155 | 1.0 | 39.4 | 153.1 | >7,500 |
| 156 | 0.4 | 9.8 | 26.2 | >7,500 |
| 157 | 0.6 | 5.9 | 21.2 | >7,500 |
| 158 | 0.9 | 32.5 | 156.1 | >7,500 |
| 159 | 0.7 | 21.6 | 86.8 | >7,500 |
| 160 | 0.2 | 4.4 | 25.3 | >7,500 |
| 161 | 0.5 | 11.9 | 123 | >7,500 |
| 162 | 0.4 | 7.7 | 660.5 | >7,500 |
| 163 | 0.3 | 3.9 | 159.8 | >7,500 |
| 164 | 1.5 | 6.2 | | >7,500 |
| 165 | 0.6 | 3.4 | 11.4 | >7,500 |
| 166 | 0.7 | 13.9 | 233.0 | >7,500 |
| 167 | 0.5 | 4.2 | 24.7 | >7,500 |
| 168 | 0.6 | 17.4 | 371.4 | >7,500 |
| 169 | 0.3 | 2.8 | 15.0 | >7,500 |
| 170 | 0.3 | 4.1 | 33.7 | >7,500 |
| 171 | 0.3 | 2.6 | 34.8 | >7,500 |
| 172 | 0.3 | 3.0 | 66.6 | >7,500 |
| 173 | 0.3 | 3.1 | 11.8 | >7,500 |
| 174 | 0.3 | 2.5 | 9.9 | >7,500 |
| 175 | 0.5 | 3.8 | 16.6 | >7,500 |
| 176 | 0.5 | 3.1 | 29.1 | >7,500 |
| 177 | 0.3 | 2.6 | 18.2 | >7,500 |
| 178 | 0.2 | 1.8 | 18.7 | >7,500 |
| 179 | 1.0 | 6.9 | 54.5 | >7,500 |
| 180 | 0.4 | 6.1 | 38.4 | >7,500 |
| 181 | 0.2 | 2.3 | 16.1 | >7,500 |
| 182 | 24.1 | 1982.9 | 1679.2 | >7,500 |
| 183 | 11.1 | 652.1 | 178.0 | >7,500 |
| 184 | 15.9 | 828.7 | 1632.7 | >7,500 |
| 185 | 703.6 | >3000 | 42.6 | >7,500 |
| 186 | 41.4 | 2254.8 | 593.2 | >7,500 |
| 187 | 27.7 | 1187.7 | 1023.3 | >7,500 |
| 188 | 9.5 | 682.8 | 347.5 | >7,500 |
| 189 | 7.3 | 319.1 | 72.3 | >7,500 |
| 190 | 6.0 | 397.7 | 147.4 | >7,500 |
| 191 | 248.5 | >3000 | >3000 | >7,500 |
| 192 | 34.6 | 1383.9 | >3000 | >7,500 |
| 193 | 321.1 | >3000 | >3000 | >7,500 |
| 194 | 147.3 | >3000 | >3000 | 2,036 |
| 195 | 156.5 | >3000 | >3000 | >7,500 |
| 196 | 1.4 | 119.3 | 85.8 | >7,500 |
| 197 | 11.5 | 51 | 612.9 | >7,500 |
| 198 | 2.0 | 110.6 | 105.2 | >7,500 |
| 199 | 2.0 | 125.4 | 237.1 | >7,500 |
| 200 | 49.0 | 2206.0 | >3000 | >7,500 |
| 201 | 58.5 | 2451.9 | 1435.8 | >7,500 |
| 202 | 1073.5 | >3000 | >3000 | >7,500 |
| 203 | 25.0 | 1060.7 | 449.1 | >7,500 |
| 204 | 19.2 | 419.6 | 460.4 | >7,500 |
| 205 | 39.4 | 107 | 561.8 | >7,500 |
| 206 | 3551.4 | >3000 | >3000 | >2,500 |
| 207 | 0.3 | 4.4 | 3.3 | >7,500 |
| 208 | 0.3 | 4.3 | 96.0 | >7,500 |
| 209 | 0.3 | 2.2 | 2.8 | >7,500 |
| 210 | 0.3 | 2.6 | 89.8 | >7,500 |
| 211 | 0.3 | 2.4 | 63.6 | >7,500 |
| 212 | 0.3 | 1.3 | 103.7 | >7,500 |
| 213 | 0.3 | 1.9 | 2.4 | >7,500 |
| 214 | 0.3 | 1.8 | 3.9 | >7,500 |
| 215 | 0.5 | 16.8 | 80.1 | >7,500 |
| 216 | 1.5 | 47.8 | 625.5 | >7,500 |
| 217 | 0.4 | 2.5 | 8.9 | >7,500 |
| 218 | 0.3 | 3.6 | 78.8 | >7,500 |
| 219 | 0.4 | 14.0 | 122.7 | >7,500 |
| 220 | 0.2 | 5.0 | 59.0 | >7,500 |
| 221 | 0.9 | 7.1 | 1710.4 | >7,500 |
| 222 | 0.7 | 16.5 | 988.8 | >7,500 |
| 223 | 0.6 | 5.7 | 69.5 | >833 |
| 224 | 1.4 | 11.2 | 902.6 | >2,500 |
| 225 | 0.6 | 12.2 | >2000 | >7,500 |
| 226 | 0.2 | 1.3 | 3.2 | >2,500 |
| 227 | 0.9 | 21.3 | 437.8 | >833 |
| 228 | 0.2 | 1.5 | 5.6 | >833 |
| 229 | 0.2 | 1.6 | 15.5 | >833 |
| 230 | 3.2 | 82.9 | 791.4 | >2,500 |
| 231 | 0.4 | 3.3 | 73.1 | >7,500 |
| 232 | 25.9 | 750.9 | 1726.6 | >7,500 |
| 233 | 0.2 | 1.1 | 1.3 | >7,500 |
| 234 | 4.6 | 283.2 | >3000 | >7,500 |
| 235 | 0.5 | 5.2 | 9.5 | >7,500 |
| 236 | 2.1 | 83.6 | 676.7 | >2,500 |
| 237 | 1.4 | 57.2 | 618.6 | >7,500 |
| 238 | 0.4 | 7.5 | 181.4 | >2,500 |
| 239 | 5.0 | 78.7 | 348.6 | >2,500 |
| 240 | 0.3 | 2.0 | 11.8 | >2,500 |
| 241 | 0.1 | 0.7 | 2.7 | >2,500 |
| 242 | 0.2 | 1.7 | 6.1 | >7,500 |
| 243 | 0.5 | 13.1 | 20.3 | >7,500 |
| 244 | 0.5 | 15.3 | 22.9 | >7,500 |
| 245 | 0.4 | 10.8 | 18.1 | >7,500 |
| 246 | 1.4 | 64.7 | 113.3 | >7,500 |
| 247 | 0.6 | 19.3 | 18.3 | >7,500 |
| 248 | 0.4 | 10.2 | 31.3 | >7,500 |
| 249 | 0.5 | 6.5 | 59.8 | >7,500 |
| 250 | 1.4 | 75.2 | 299.2 | >7,500 |
| 251 | 1.5 | 33.6 | 28.4 | >7,500 |
| 252 | 1.1 | 53.2 | 79.7 | >7,500 |
| 253 | 0.2 | 1.8 | 4.4 | >7,500 |
| 254 | 0.2 | 2.6 | 6.2 | >7,500 |
| 255 | 0.2 | 1.9 | 9.3 | >7,500 |
| 256 | 0.6 | 14.9 | 1344.0 | >7,500 |
| 257 | 0.2 | 2.6 | 13.1 | >2,500 |
| 258 | 0.4 | 4.2 | 48.7 | >7,500 |
| 259 | 0.4 | 2.8 | 3.9 | >7,500 |
| 260 | 0.3 | 2.1 | 3.1 | >7,500 |
| 261 | 0.4 | 4.9 | 4.2 | >7,500 |
| 262 | 0.5 | 6.1 | 36.9 | >7,500 |
| 263 | 0.6 | 7.9 | 13.9 | >7,500 |
| 264 | 0.4 | 6.1 | 7.6 | >7,500 |
| 265 | 0.5 | 8.7 | 113.9 | >7,500 |
| 266 | 0.7 | 11.1 | 36.2 | >7,500 |
| 267 | 0.9 | 13.7 | 701.0 | >7,500 |
| 268 | 0.6 | 14.5 | 11.6 | >7,500 |
| 269 | 1.8 | 71.2 | 17.5 | >7,500 |
| 270 | 0.4 | 5.4 | 4.8 | >7,500 |
| 271 | 0.2 | 2.8 | 10.3 | 5930 |
| 272 | 0.2 | 1.4 | 5.6 | >7,500 |
| 273 | 0.2 | 3.6 | 13.7 | >7,500 |
| 274 | 1.0 | 21.4 | 51.8 | >7,500 |
| 275 | 0.2 | 2.5 | 7.6 | >7,500 |
| 277 | 0.4 | 5.7 | 141.6 | >2,500 |
| 278 | 0.2 | 1.4 | 1.5 | >7,500 |
| 279 | 0.4 | 8.6 | 134.0 | >7,500 |
| 280 | 0.3 | 4.3 | 85.9 | >7,500 |
| 281 | 0.4 | 3.8 | 10.9 | >7,500 |
| 282 | 0.1 | 1.2 | 0.9 | 2680 |
| 283 | 0.1 | 2.2 | 1.5 | >7,500 |
| 284 | 0.2 | 2.6 | 2.8 | >7,500 |
| 285 | 0.1 | 1.3 | 1.2 | >7,500 |
| 286 | 0.1 | 1.0 | 1.4 | >2,500 |
| 287 | 0.1 | 0.8 | 1.8 | 1740 |
| 288 | 0.4 | 11.5 | 13.5 | >2,500 |
| 289 | 0.3 | 7.1 | 4.4 | >7,500 |
| 290 | 0.4 | 3.3 | 19.5 | >7,500 |
| 292 | 0.3 | 2.2 | 4.2 | >7,500 |
| 293 | 0.4 | 4.6 | 337.1 | >7,500 |
| 294 | 0.3 | 5.1 | 12.7 | >7,500 |
| 295 | 0.7 | 13.5 | 21.3 | >7,500 |
| 296 | 0.3 | 3.7 | 7.6 | >7,500 |
| 297 | 0.7 | 8.6 | 98.0 | >7,500 |
| 298 | 0.9 | 6.4 | 236.0 | >7,500 |
| 299 | 0.5 | 6.0 | 101.5 | >7,500 |
| 300 | 1.2 | 12.4 | 1013.9 | >7,500 |
| 301 | 0.2 | 1.4 | 4.3 | >7,500 |
| 302 | 1.2 | 61.2 | 7 | >7,500 |
| 303 | 0.4 | 5.8 | 120.9 | >7,500 |
| 304 | 0.6 | 5.6 | 9.3 | >7,500 |
| 305 | 2.1 | 57.8 | 307.7 | >7,500 |
| 306 | 0.5 | 9.0 | 4.8 | >7,500 |
| 307 | 9.7 | 209.5 | 73.2 | >7,500 |
| 308 | 20.9 | 710.2 | 311.2 | >7,500 |
| 309 | 0.2 | 1.5 | 4.2 | >7,500 |
| 310 | 0.3 | 1.4 | 3.3 | >7,500 |
| 311 | 0.2 | 1.3 | 2.4 | >7,500 |
| 312 | 1.8 | 39.2 | 947.5 | >7,500 |
| 313 | 0.2 | 2.6 | 2.8 | >7,500 |
| 314 | 2.5 | 64.4 | 547.5 | >7,500 |
| 315 | 0.3 | 4.6 | 5.4 | 4990 |
| 316 | 1.9 | 21.7 | 58.3 | >7,500 |
| 317 | 0.3 | 2.5 | 6.5 | >7,500 |
| 318 | 0.3 | 1.9 | 3.8 | >7,500 |
| 319 | 0.3 | 1.8 | 2.7 | >7,500 |
| 320 | 0.2 | 1.4 | 1.6 | >7,500 |
| 321 | 0.2 | 1.0 | 1.2 | >7,500 |
| 322 | 0.7 | 15.5 | 122.0 | >7,500 |
| 323 | 0.2 | 1.7 | 2.8 | >7,500 |
| 324 | 0.1 | 0.7 | 1.1 | 5240 |
| 325 | 0.8 | 16.3 | 10.7 | >7,500 |
| 326 | 0.4 | 3.7 | 3.0 | >7,500 |
| 327 | 0.8 | 13.8 | 9.9 | >7,500 |
| 328 | 0.6 | 10.2 | 7.1 | >7,500 |
| 329 | 0.5 | 5.6 | 4.4 | >7,500 |
| 330 | 0.6 | 9.6 | 6.8 | >7,500 |
| 331 | 0.4 | 3.3 | 2.7 | >7,500 |
| 332 | 0.6 | 4.1 | 4.5 | >7,500 |
| 333 | 0.4 | 3.1 | 3.3 | >7,500 |
| 334 | 0.5 | 9.1 | 11.7 | >7,500 |
| 335 | 0.4 | 3.0 | 3.5 | >7,500 |
| 336 | 0.4 | 3.3 | 3.1 | >7,500 |
| 337 | 1.8 | 46.2 | 24.9 | >7,500 |
| 338 | 0.4 | 4.8 | 4.6 | >7,500 |
| 339 | 0.4 | 3.0 | 2.6 | >7,500 |
| 340 | 0.5 | 7.5 | 6.5 | >7,500 |
| 341 | 0.4 | 4.6 | 5.1 | >7,500 |
| 342 | 0.6 | 15.9 | 338.8 | >7,500 |
| 343 | 3.5 | 84.8 | 47.9 | >7,500 |
| 344 | 4.3 | 67.5 | 59.2 | >7,500 |
| 345 | 1.3 | 56.9 | 221.7 | >7,500 |
| 346 | 0.5 | 6.5 | 6.8 | >7,500 |
| 347 | 0.5 | 6.0 | 6.0 | >7,500 |
| 348 | 0.4 | 4.0 | 195.1 | >7,500 |
| 349 | 0.6 | 8.7 | 9.1 | >7,500 |
| 350 | 0.3 | 4.1 | 73.3 | 6350 |
| 351 | 0.3 | 3.1 | 17.7 | >7,500 |
| 352 | 0.3 | 4.7 | 2.8 | >7,500 |
| 353 | 0.2 | 1.4 | 1.6 | >7,500 |
| 354 | 0.2 | 4.3 | 78.8 | >7,500 |
| 355 | 0.4 | 11.3 | 50.3 | >7,500 |
| 356 | 0.5 | 17.9 | 74.6 | >7,500 |
| 357 | 0.2 | 4.5 | 56.9 | >7,500 |
| 358 | 86.6 | 1837.4 | 940.6 | >7,500 |
| 359 | 16.4 | 451.3 | 365.2 | >7,500 |
| 360 | 4.9 | 100.3 | 59.2 | >7,500 |
| 361 | 1.3 | 19.0 | 14.9 | >7,500 |
| 362 | 0.5 | 4.5 | 62.3 | >2,500 |
| 363 | 1.3 | 14.1 | >2000 | >7,500 |
| 364 | 0.8 | 12.2 | 10.3 | >7,500 |
| 365 | 0.3 | 2.4 | 4.8 | 596 |
| 366 | 0.3 | 2.5 | 7.7 | 571 |
| 367 | 1.8 | 17.3 | 18.6 | >7,500 |
| 368 | 0.3 | 2.4 | 3.7 | >7,500 |
| 369 | 0.2 | 1.4 | 1.6 | >7,500 |
| 370 | 1.0 | 10.1 | 543.9 | >7,500 |
| 371 | 0.4 | 3.0 | 29.3 | >2,500 |
| 372 | 0.6 | 7.7 | 21.2 | >2,500 |
| 373 | 1.3 | 7.0 | 56.7 | >7,500 |
| 374 | 0.4 | 4.0 | 41.3 | >7,500 |
| 375 | 0.6 | 4.5 | 10.2 | >833 |
| 376 | 0.8 | 5.6 | 153.7 | >7,500 |
| 377 | 0.8 | 6.2 | 118.9 | >7,500 |
| 378 | 0.8 | 10.2 | 304.2 | >2,500 |
| 379 | 0.4 | 2.7 | 9.2 | >2,500 |
| 380 | 0.4 | 3.0 | 9.8 | >2,500 |
| 381 | 0.4 | 2.5 | 16.0 | >833 |
| 382 | 0.6 | 5.3 | 7.6 | >2,500 |
| 383 | 1.0 | 7.8 | 15.0 | >2,500 |
| 384 | 0.4 | 2.3 | 4.3 | >833 |
| 385 | 0.8 | 4.9 | 9.5 | >833 |
| 386 | 0.4 | 2.5 | 3.8 | >833 |
| 387 | 0.2 | 1.5 | 6.9 | >7,500 |
| 388 | 0.5 | 7.4 | 5.1 | >7,500 |
| 389 | 0.6 | 7.5 | 3.1 | >7,500 |
| 390 | 0.6 | 10.1 | 5.5 | >7,500 |
| 391 | 0.9 | 18.4 | 10.4 | >7,500 |
| 392 | 0.5 | 4.6 | 4.8 | 7440 |
| 393 | 0.7 | 5.1 | 3.5 | >7,500 |
| 394 | 0.2 | 1.2 | 1.8 | >7,500 |
| 395 | 0.4 | 7.3 | 6.2 | >7,500 |
| 396 | 0.3 | 1.8 | 4.3 | >7,500 |
| 397 | 0.8 | 22.1 | 7.6 | >7,500 |
| 398 | 6.8 | 180.9 | 169.0 | >7,500 |
| 399 | 0.2 | 1.1 | 1.3 | 4770 |
| 400 | 0.4 | 6.4 | 14.8 | >7,500 |
| 401 | 0.3 | 4.3 | 10.3 | >7,500 |
| 402 | 0.3 | 2.7 | 5.4 | >7,500 |
| 403 | 0.2 | 1.3 | 3.3 | >7,500 |
| 404 | 0.3 | 4.9 | 7.2 | >7,500 |
| 405 | 0.2 | 3.9 | 3.8 | 6660 |
| 406 | 0.4 | 2.3 | 18.9 | >7,500 |
| 407 | 0.3 | 6.2 | 11.9 | >7,500 |
| 408 | 0.2 | 1.8 | 1.7 | >7,500 |
| 409 | 0.2 | 1.3 | 1.6 | 3370 |
| 410 | 6.2 | 190.2 | 269.9 | >7,500 |
| 411 | 0.5 | 6.6 | 114.1 | >7,500 |
| 412 | 1.4 | 34.6 | 77.2 | >7,500 |
| 413 | 0.3 | 2.9 | 3.4 | >7,500 |
| 414 | 0.4 | 3.3 | 6.2 | 5100 |
| 415 | 0.5 | 6.6 | 41.5 | >7,500 |
| 416 | 0.2 | 1.6 | 2.3 | >7,500 |
| 417 | 0.4 | 2.8 | 2.6 | >7,500 |
| 418 | 0.5 | 2.9 | 6.5 | >7,500 |
| 419 | 0.3 | 2.8 | 2.6 | >7,500 |
| 420 | 0.2 | 2.1 | 2.1 | >7,500 |
| 421 | 0.2 | 2.0 | 6.2 | >7,500 |
| 422 | 0.2 | 1.1 | 2.6 | >7,500 |
| 423 | 0.4 | 3.7 | 15.8 | >7,500 |
| 424 | 0.3 | 2.2 | 6.8 | >7,500 |
| 425 | 0.7 | 6.8 | 11.7 | >7,500 |
| 426 | 11.5 | 482.1 | >2000 | >7,500 |
| 427 | 2.1 | 149.5 | >2000 | >7,500 |
| 428 | 0.8 | 17.3 | 7.9 | >7,500 |
| 429 | 6.3 | 184.8 | 30.9 | >7,500 |
| 430 | 0.2 | 1.8 | 1.0 | 5170 |
| 431 | 0.5 | 3.5 | 5.0 | >7,500 |
| 432 | 1.1 | 9.2 | 419.7 | >2,500 |
| 433 | 0.5 | 4.0 | 5.1 | >7,500 |
| 434 | 0.2 | 2.2 | 2.4 | 5220 |
| 435 | 0.6 | 6.8 | 139.0 | >7,500 |
| 436 | 0.1 | 0.9 | 1.6 | 4300 |
| 437 | 0.4 | 4.6 | 6.0 | >7,500 |
| 438 | 0.4 | 4.3 | 3.7 | >7,500 |
| 439 | 0.4 | 5.0 | 12.2 | >7,500 |
| 440 | 0.4 | 2.2 | 1.6 | >7,500 |
| 441 | 0.7 | 19.7 | 30.5 | >7,500 |
| 442 | 0.3 | 3.1 | 8.1 | >7,500 |
| 443 | 0.4 | 4.4 | 3.9 | >7,500 |
| 444 | 0.2 | 1.8 | 1.3 | >7,500 |
| 445 | 0.3 | 2.1 | 2.2 | >7,500 |
| 446 | 0.3 | 3.5 | 3.0 | >7,500 |
| 447 | 0.5 | 3.7 | 12.4 | >7,500 |
| 448 | 0.2 | 1.8 | 2.4 | >7,500 |
| 449 | 0.7 | 9.1 | 10.6 | >7,500 |
| 450 | 0.4 | 2.4 | 21.6 | >7,500 |
| 451 | 0.8 | 16.1 | 67 | >7,500 |
| 452 | 12.1 | 375.9 | 91.6 | >7,500 |
| 453 | 0.6 | 5.8 | 25.1 | >7,500 |
| 454 | 0.4 | 4.6 | 12.7 | >7,500 |
| 455 | 0.6 | 3.3 | 15 | >7,500 |
| 456 | 0.4 | 4.7 | 11 | >7,500 |
| 457 | 0.4 | 3.4 | 4.0 | >7,500 |
| 458 | 0.3 | 1.7 | 2.0 | >7,500 |
| 459 | 0.3 | 1.5 | 33.7 | >7,500 |
| 460 | 0.3 | 1.6 | 13.4 | >7,500 |
| 461 | 0.2 | 0.8 | 2.1 | 3,349 |
| 462 | 0.7 | 15.3 | 298.3 | >7,500 |
| 463 | 0.7 | 11.0 | 782.4 | >7,500 |
| 464 | 5.2 | 144.8 | 1,998 | >7,500 |
| 465 | 3.0 | 73.9 | 1,863 | >7,500 |
| 466 | 9.0 | 261.1 | 349.5 | >7,500 |
| 467 | 0.4 | 3.0 | 4.4 | >7,500 |
| 468 | 2.1 | 149.4 | 2,000 | >7,500 |
| 469 | 0.3 | 5.5 | 40.5 | >7,500 |
| 470 | 0.5 | 6.8 | 109.4 | >7,500 |
| 471 | 0.1 | 1.0 | 3.4 | >7,500 |
| 472 | 0.4 | 2.8 | 5.4 | >7,500 |
| 473 | 0.4 | 4.8 | 5.3 | >7,500 |
| 474 | 0.2 | 1.7 | 3.9 | >7,500 |
| 475 | 0.2 | 1.4 | 2.3 | >7,500 |
| 476 | 0.2 | 1.7 | 4.9 | >7,500 |
| 477 | 0.3 | 2.3 | 45.5 | >7,500 |
| 478 | 0.3 | 3.0 | 4.6 | 2,559 |
| 479 | 0.3 | 2.0 | 97.1 | >7,500 |
| 480 | 0.3 | 2.5 | 19.5 | >7,500 |
| 481 | 0.3 | 2.0 | 3.9 | >7,500 |
| 482 | 0.2 | 1.6 | 28.6 | >7,500 |
| 483 | 0.3 | 2.1 | 17.7 | >7,500 |
| 484 | 0.2 | 1.4 | 44.1 | >7,500 |
| 485 | 0.8 | 16.3 | 34.4 | >7,500 |
| 486 | 2.6 | 127.2 | 1,998 | >7,500 |
| 487 | 7.7 | 196.6 | 42.6 | >7,500 |
| 488 | 92.3 | 1,279 | 374.9 | >7,500 |
| 489 | 0.5 | 5.5 | | >7,500 |
| 490 | 0.3 | 2.3 | | >7,500 |
| 491 | 0.3 | 4.9 | | >7,500 |
| 492 | 0.7 | 15.0 | | >7,500 |
| 493 | 0.2 | 1.9 | 7.0 | >7,500 |
| 494 | 0.4 | 3.9 | 9.6 | >7,500 |
| 495 | 0.3 | 2.5 | 3.8 | >7,500 |
| 496 | 0.3 | 2.8 | 5.6 | >7,500 |

In some embodiments, the disclosure is directed to the following aspects:
Aspect 1. A compound of formula (I):

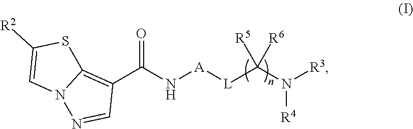

or pharmaceutically acceptable salts thereof, wherein A in the compound of formula (I) is an optionally substituted phenyl ring, an optionally substituted pyridinyl ring, or an optionally substituted 5-membered heteroaryl ring containing 1-2 heteroatoms that are each independently O, N, or S; R² is optionally substituted aryl, optionally substituted heteroaryl, optionally substituted alkyl, optionally substituted cycloalkyl, or optionally substituted heterocycloalkyl; R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system optionally includes, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; n is 1, 2, or 3; and when n is 1, L is —NHC(O)—, and when n is 2 or 3, L is —C(O)NH—, —NHC(O)—, or —NHC(O)NH.
Aspect 2. The compound of formula (I) according to aspect 1, wherein said compound is a compound of formula (IA) or formula (IB):

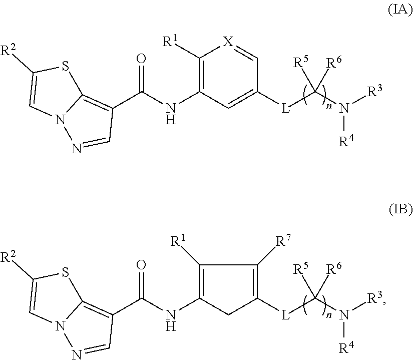

or a pharmaceutically acceptable salt thereof,
wherein
R¹ is H, C₁-C₆alkyl, C₃-C₆cycloalkyl, halogen, —CN, or C₁-C₄fluoroalkyl;
R⁷ is H, C₁-C₆alkyl, C₃-C₆cycloalkyl, halogen, —CN, or —CF₃; and
X is N, or CH.
Aspect 3. The compound according to aspect 2, wherein said compound is a compound of formula (IA).
Aspect 4. The compound according to aspect 3, wherein X is N.
Aspect 5. The compound according to aspect 3, wherein X is CH.
Aspect 6. The compound according to aspect 2, wherein said compound is a compound of formula (IB).
Aspect 7. The compound according to any one of the preceding aspects, wherein R¹ is C₁-C₆alkyl.
Aspect 8. The compound according to aspect 7, wherein the C₁-C₆alkyl is a C₁-C₄alkyl.
Aspect 9. The compound according to aspect 8, wherein the C₁-C₄alkyl is —CH₃.
Aspect 10. The compound according to any one of the preceding aspects, wherein R² is optionally substituted heteroaryl.
Aspect 11. The compound according to aspect 10, where said optionally substituted heteroaryl is substituted with an optionally substituted C₁-C₆alkyl, or an optionally substituted C₃-C₅cycloalkyl.
Aspect 12. The compound according to aspect 10, wherein said optionally substituted heteroaryl is an optionally substituted 5-membered heteroaryl.
Aspect 13. The compound according to aspect 12, where said optionally substituted 5-membered heteroaryl is substituted with an optionally substituted C₁-C₆alkyl, or an optionally substituted C₃-C₅cycloalkyl.
Aspect 14. The compound according to aspect 12, wherein said optionally substituted 5-membered heteroaryl is an optionally substituted pyrazolyl.
Aspect 15. The compound according to aspect 14, wherein said optionally substituted pyrazolyl is substituted with an optionally substituted C₁-C₆alkyl, or an optionally substituted C₃-C₅cycloalkyl.
Aspect 16. The compound according to aspect 15, wherein said optionally substituted pyrazolyl is 1-methyl-pyrazol-3-yl, 1-methyl-pyrazol-4-yl, 1-methyl-pyrazol-5-yl, 1-cyclopropyl-pyrazol-3-yl, 1-cyclopropyl-pyrazol-4-yl, 1-cyclopropyl-pyrazol-5-yl, 1-(2-hydroxyethyl)-pyrazol-3-yl, 1-(2-hydroxyethyl)-pyrazol-4-yl, or 1-(2-hydroxyethyl)-pyrazol-5-yl.
Aspect 17. The compound according to aspect 15, wherein said optionally substituted pyrazolyl is 1-(2-methoxyethyl)-1H-pyrazol-4-yl.
Aspect 18. The compound according to aspect 10, wherein said optionally substituted heteroaryl is an optionally substituted 6-membered heteroaryl.
Aspect 19. The compound according to aspect 18, where said optionally substituted 6-membered heteroaryl is substituted with an optionally substituted C₁-C₆alkyl, or an optionally substituted C₃-C₅cycloalkyl.
Aspect 20. The compound according to aspect 18, wherein said optionally substituted 6-membered heteroaryl is an optionally substituted pyridinyl.
Aspect 21. The compound according to aspect 20, wherein said pyridinyl is not substituted.
Aspect 22. The compound according to aspect 20, wherein said pyridinyl is C₁-C₆alkoxyl substituted pyridinyl.
Aspect 23. The compound according to aspect 22, wherein said pyridinyl is 4-methoxypyridin-3-yl.
Aspect 24. The compound according to aspect 10, wherein said optionally substituted heteroaryl is an optionally substituted 6,7-dihydro-5H-pyrazolo[5,1-b][1,3]oxazine.
Aspect 25. The compound according to aspect 24, wherein said optionally substituted heteroaryl is 6,7-dihydro-5H-pyrazolo[5,1-b][1,3]oxazin-3-yl.
Aspect 26. The compound according to any one of aspects 10-25, wherein R¹ is C₁-C₆alkyl.
Aspect 27. The compound according to aspect 26, wherein said C₁-C₆alkyl is —CH₃.
Aspect 28. The compound according to any one of the preceding aspects, wherein L is —C(O)NH—.
Aspect 29. The compound according to any one of aspects 1-27, wherein L is —NHC(O)—.
Aspect 30. The compound according to any one of aspects 1-27, wherein L is or —NHC(O)NH—.
Aspect 31. The compound according to aspect 29, wherein n is 1.
Aspect 32. The compound according to any one of aspects 1-30, wherein n is 2.
Aspect 33. The compound according to any one of aspects 1-30, wherein n is 3.
Aspect 34. The compound according to any one of the preceding aspects, wherein each R⁵ and each R⁶ is H.
Aspect 35. The compound according to any one of the preceding aspects, wherein R³ is optionally substituted alkyl and R⁴ is optionally substituted heterocycloalkyl.
Aspect 36. The compound according to aspect 35, wherein R³ is —CH₃ and R⁴ is tetrahydropyran-4-yl.
Aspect 37. The compound according to any one of the preceding aspects, wherein R³ and R⁴, together with the nitrogen atom to which they are both attached, form an optionally substituted 3-12-membered heterocycloalkyl ring, an optionally substituted 5-12-membered bridged heterocycloalkyl ring; an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, and 5-12-membered spiroheterocycloalkyl ring system optionally includes, in addition to the nitrogen atom to which both R³ and R⁴ are attached, 1-3 heteroatoms that are each independently O, S, or N.
Aspect 38. The compound according to aspect 37, wherein R³ and R⁴, together with the nitrogen atom to which they are both attached, form an optionally substituted 3-12-membered heterocycloalkyl ring.
Aspect 39. The compound according to aspect 38, wherein the optionally substituted 3-12-membered heterocycloalkyl ring is substituted with at least one C₁-C₆alkyl group.
Aspect 40. The compound according to aspect 39, wherein the C₁-C₆alkyl group is a methyl group.
Aspect 41. The compound according to any one of aspects 37-40, wherein R³ and R⁴, together with the nitrogen atom to which they are both attached, form a 2,2-dimethylpyrrolidin-1-yl group.
Aspect 42. The compound according to any one of aspects 37-40, wherein R³ and R⁴, together with the nitrogen atom to which they are both attached, form a 3,3-dimethylpyrrolidin-1-yl.
Aspect 43. The compound according to any one of aspects 37-40, wherein R³ and R⁴, together with the nitrogen atom to which they are both attached, form a 3,3-dimethylazetidin-1-yl.
Aspect 44. The compound according to any one of aspects 37-40, wherein R³ and R⁴, together with the nitrogen atom to which they are both attached, form a 2-methyl-pyrrolidin-1-yl.
Aspect 45. The compound according to aspect 44, wherein said 2-methyl-pyrrolidin-1-yl is (R)-2-methyl-pyrrolidin-1-yl.
Aspect 46. The compound according to aspect 44, wherein said 2-methyl-pyrrolidin-1-yl is (S)-2-methyl-pyrrolidin-1-yl.
Aspect 47. The compound according to any one of aspects 37-40, wherein R³ and R⁴, together with the nitrogen atom to which they are both attached, form a 2-methyl-piperidin-1-yl.
Aspect 48. The compound according to any one of aspects 37-40, wherein R³ and R⁴, together with the nitrogen atom to which they are both attached, form a (R)-2-methyl-piperidin-1-yl.
Aspect 49. The compound according to any one of aspects 37-40, wherein R³ and R⁴, together with the nitrogen atom to which they are both attached, form a (S)-2-methyl-piperidin-1-yl.
Aspect 50. The compound according to aspect 38, wherein the optionally substituted 3-12-membered heterocycloalkyl ring is substituted with at least one halogen atom.
Aspect 51. The compound according to aspect 50, wherein the halogen atom is —F.
Aspect 52. The compound according to any one of aspects 38, 50, or 51, wherein R³ and R⁴, together with the nitrogen atom to which they are both attached, form a 4,4-difluoropiperidin-1-yl.
Aspect 53. The compound according to aspect 37, wherein R³ and R⁴, together with the nitrogen atom to which they are both attached, form an optionally substituted 5-12-membered bridged heterocycloalkyl ring.
Aspect 54. The compound according to aspect 53, wherein R³ and R⁴, together with the nitrogen atom to which they are both attached, form an 2-azabicyclo[2.2.2]octan-2-yl.
Aspect 55. The compound according to aspect 53, wherein R³ and R⁴, together with the nitrogen atom to which they are both attached, form an 9-azabicyclo[3.3.1]nonan-9-yl.
Aspect 56. The compound according to aspect 53, wherein R³ and R⁴, together with the nitrogen atom to which they are both attached, form an 3-azabicyclo[3.1.1]heptan-3-yl.
Aspect 57. The compound according to aspect 37, wherein R³ and R⁴, together with the nitrogen atom to which they are both attached, form an optionally substituted 5-12-membered spiroheterocycloalkyl ring.
Aspect 58. The compound according to aspect 57, wherein said optionally substituted 5-12-membered spiroheterocycloalkyl ring is 4-azaspiro[2.4]heptan-4-yl.
Aspect 59. The compound according to aspect 57, wherein said optionally substituted 5-12-membered spiroheterocycloalkyl ring is 5-azaspiro[3.4]octan-5-yl.
Aspect 60. The compound according to aspect 57, wherein said optionally substituted 5-12-membered spiroheterocycloalkyl ring is 6-azaspiro[3.4]octan-6-yl.
Aspect 61. The compound according to aspect 57, wherein said optionally substituted 5-12-membered spiroheterocycloalkyl ring is 6-azaspiro[2.5]octan-6-yl.
Aspect 62. The compound according to aspect 57, wherein said optionally substituted 5-12-membered spiroheterocycloalkyl ring is 5-azaspiro[2.4]heptan-5-yl.
Aspect 63. The compound according to aspect 57, wherein said optionally substituted 5-12-membered spiroheterocycloalkyl ring is 2-oxa-5-azaspiro[3.5]nonan-5-yl.
Aspect 64. The compound according to aspect 57, wherein said optionally substituted 5-12-membered spiroheterocycloalkyl ring is 2-oxa-6-azaspiro[3.5]nonan-6-yl.
Aspect 65. The compound according to aspect 57, wherein said optionally substituted 5-12-membered spiroheterocycloalkyl ring is 7-azaspiro[4.4]nonan-7-yl.
Aspect 66. The compound according to aspect 57, wherein said optionally substituted 5-12-membered spiroheterocycloalkyl ring is 2-oxa-5-azaspiro[3.4]octan-5-yl.
Aspect 67. The compound according to aspect 57, wherein said optionally substituted 5-12-membered spiroheterocycloalkyl ring is 2-oxa-6-azaspiro[3.4]octan-6-yl.
Aspect 68. The compound according to aspect 37, wherein R³ and R⁴, together with the nitrogen atom to which they are both attached, form an optionally substituted 4-12-membered fused heterocycloalkyl ring system.
Aspect 69. The compound according to aspect 68, wherein the optionally substituted 4-12-membered fused heterocycloalkyl ring system is 3,4-dihydro-2,7-naphthyridin-2 (1H)-yl.
Aspect 70. The compound according to aspect 68, wherein the optionally substituted 4-12-membered fused heterocycloalkyl ring system is 1-methyl-4,5-dihydro-1H-pyrazolo[3,4-c]pyridin-6 (7H)-yl.
Aspect 71. The compound according to aspect 1 or aspect 2, wherein said compound is:
N-(5-((2-(2-azabicyclo[2.2.2]octan-2-yl)ethyl)carb-amoyl)-2-methylpyridin-3-yl)-2-(1-methyl-1H-pyrazol-4-yl)pyrazolo[5,1-b]thiazole-7-carboxamide;
N-(5-((2-(4-azaspiro[2.4]heptan-4-yl)ethyl)carbamoyl)-2-methylpyridin-3-yl)-2-(1-methyl-1H-pyrazol-4-yl)pyrazolo[5,1-b]thiazole-7-carboxamide;
N-(5-((2-(5-azaspiro[3.4]octan-5-yl)ethyl)carbamoyl)-2-methylpyridin-3-yl)-2-(1-methyl-1H-pyrazol-4-yl)pyrazolo[5,1-b]thiazole-7-carboxamide;
N-(5-((2-(9-azabicyclo[3.3.1]nonan-9-yl)ethyl)carbamoyl)-2-methylpyridin-3-yl)-2-(1-methyl-1H-pyrazol-4-yl)pyrazolo[5,1-b]thiazole-7-carboxamide;
N-(5-((2-(3-azabicyclo[3.1.1]heptan-3-yl)ethyl)carbamoyl)-2-methylpyridin-3-yl)-2-(1-methyl-1H-pyrazol-4-yl)pyrazolo[5,1-b]thiazole-7-carboxamide;
2-(1-cyclopropyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide;
N-(5-((2-(2,2-dimethylpyrrolidin-1-yl)ethyl)carbamoyl)-2-methyl-pyridin-3-yl)-2-(pyridin-4-yl)pyrazolo[5,1-b]thiazole-7-carboxamide;
N-(5-((2-(2,2-dimethylpyrrolidin-1-yl)ethyl)carbamoyl)-2-methylpyridin-3-yl)-2-(1-methyl-1H-pyrazol-4-yl)pyrazolo[5,1-b]thiazole-7-carboxamide;
N-(5-((2-(2,2-dimethylpyrrolidin-1-yl)ethyl)carbamoyl)-2-methylpyridin-3-yl)-2-(1-methyl-1H-pyrazol-3-yl)pyrazolo[5,1-b]thiazole-7-carboxamide;
N-(5-(3-(2,2-dimethylpyrrolidin-1-yl)propanamido)-2-methyl-pyridin-3-yl)-2-(1-methyl-1H-pyrazol-4-yl)pyrazolo[5,1-b]thiazole-7-carboxamide;
N-(5-(3-(2,2-dimethylpyrrolidin-1-yl)propanamido)-2-methyl-pyridin-3-yl)-2-(1-methyl-1H-pyrazol-5-yl)pyrazolo[5,1-b]thiazole-7-carboxamide;
N-(5-(3-(2,2-dimethylpyrrolidin-1-yl)propanamido)-2-methyl-pyridin-3-yl)-2-(1-methyl-1H-pyrazol-3-yl)pyrazolo[5,1-b]thiazole-7-carboxamide;
N-(5-(2-(2,2-dimethylpyrrolidin-1-yl)acetamido)-2-methylpyridin-3-yl)-2-(1-methyl-1H-pyrazol-4-yl)pyrazolo[5,1-b]thiazole-7-carboxamide;
N-(5-(2-(4,4-difluoropiperidin-1-yl)acetamido)-2-methylpyridin-3-yl)-2-(1-methyl-1H-pyrazol-4-yl)pyrazolo[5,1-b]thiazole-7-carboxamide;
2-(1-methyl-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide;
N-(5-(2-(4-azaspiro[2.4]heptan-4-yl)acetamido)-2-methylpyridin-3-yl)-2-(1-methyl-1H-pyrazol-4-yl)pyrazolo[5,1-b]thiazole-7-carboxamide;
2-(1-methyl-1H-pyrazol-4-yl)-N-(2-methyl-5-(2-(1-methyl-4,5-dihydro-1H-pyrazolo[3,4-c]pyridin-6 (7H)-yl)acetamido)pyridin-3-yl)pyrazolo[5,1-b]thiazole-7-carboxamide;
N-(5-(2-(2-oxa-6-azaspiro[3.4]octan-6-yl)acetamido)-2-methylpyridin-3-yl)-2-(1-methyl-1H-pyrazol-4-yl)pyrazolo[5,1-b]thiazole-7-carboxamide;
N-(5-(2-(2-oxa-7-azaspiro[4.4]nonan-7-yl)acetamido)-2-methylpyridin-3-yl)-2-(1-methyl-1H-pyrazol-4-yl)pyrazolo[5,1-b]thiazole-7-carboxamide;
N-(5-(2-(2-oxa-5-azaspiro[3.4]octan-5-yl)acetamido)-2-methyl-pyridin-3-yl)-2-(1-methyl-1H-pyrazol-4-yl)pyrazolo[5,1-b]thiazole-7-carboxamide;
N-(5-(2-(1-oxa-7-azaspiro[4.4]nonan-7-yl)acetamido)-2-methylpyridin-3-yl)-2-(1-methyl-1H-pyrazol-4-yl)pyrazolo[5,1-b]thiazole-7-carboxamide;
N-(5-(2-(3,3-dimethylazetidin-1-yl)acetamido)-2-methylpyridin-3-yl)-2-(1-methyl-1H-pyrazol-4-yl)pyrazolo[5,1-b]thiazole-7-carboxamide;
N-(5-(2-(3,4-dihydro-2,7-naphthyridin-2 (1H)-yl)acetamido)-2-methylpyridin-3-yl)-2-(1-methyl-1H-pyrazol-4-yl)pyrazolo[5,1-b]thiazole-7-carboxamide;
N-(5-(2-(2-oxa-6-azaspiro[3.5]nonan-6-yl)acetamido)-2-methylpyridin-3-yl)-2-(1-methyl-1H-pyrazol-4-yl)pyrazolo[5,1-b]thiazole-7-carboxamide;
N-(5-(2-(2-oxa-5-azaspiro[3.5]nonan-5-yl)acetamido)-2-methylpyridin-3-yl)-2-(1-methyl-1H-pyrazol-4-yl)pyrazolo[5,1-b]thiazole-7-carboxamide;
(R)-2-(1-methyl-1H-pyrazol-4-yl)-N-(2-methyl-5-(2-(2-methyl-pyrrolidin-1-yl)acetamido)pyridin-3-yl)pyrazolo[5,1-b]thiazole-7-carboxamide;
(S)-2-(1-methyl-1H-pyrazol-4-yl)-N-(2-methyl-5-(2-(2-methyl-pyrrolidin-1-yl)acetamido)pyridin-3-yl)pyrazolo[5,1-b]thiazole-7-carboxamide;
N-(5-(2-(3,3-dimethylpyrrolidin-1-yl)acetamido)-2-methylpyridin-3-yl)-2-(1-(2-hydroxyethyl)-1H-pyrazol-4-yl)pyrazolo[5,1-b]thiazole-7-carboxamide;
N-(5-(2-(5-azaspiro[3.4]octan-5-yl)acetamido)-2-methylpyridin-3-yl)-2-(1-(2-hydroxyethyl)-1H-pyrazol-4-yl)pyrazolo[5,1-b]thiazole-7-carboxamide;
N-(5-(2-(6-azaspiro[3.4]octan-6-yl)acetamido)-2-methylpyridin-3-yl)-2-(1-(2-hydroxyethyl)-1H-pyrazol-4-yl)pyrazolo[5,1-b]thiazole-7-carboxamide;
N-(5-(2-(6-azaspiro[2.5]octan-6-yl)acetamido)-2-methylpyridin-3-yl)-2-(1-(2-hydroxyethyl)-1H-pyrazol-4-yl)pyrazolo[5,1-b]thiazole-7-carboxamide;
N-(5-(2-(5-azaspiro[2.4]heptan-5-yl)acetamido)-2-methylpyridin-3-yl)-2-(1-(2-hydroxyethyl)-1H-pyrazol-4-yl)pyrazolo[5,1-b]thiazole-7-carboxamide;
(R)-2-(1-(2-hydroxyethyl)-1H-pyrazol-4-yl)-N-(2-methyl-5-(2-(2-methyl-piperidin-1-yl)acetamido)pyridin-3-yl)pyrazolo[5,1-b]thiazole-7-carboxamide;
(S)-2-(1-(2-hydroxyethyl)-1H-pyrazol-4-yl)-N-(2-methyl-5-(2-(2-methyl-piperidin-1-yl)acetamido)pyridin-3-yl)pyrazolo[5,1-b]thiazole-7-carboxamide;
N-(5-((2-(5-azaspiro[3.4]octan-5-yl)ethyl)carbamoyl)-3-methylthiophen-2-yl)-2-(1-methyl-1H-pyrazol-4-yl)pyrazolo[5,1-b]thiazole-7-carboxamide;
N-(5-((2-(2,2-dimethylpyrrolidin-1-yl)ethyl)carbamoyl)-3-methylthiophen-2-yl)-2-(pyridin-3-yl)pyrazolo[5,1-b]thiazole-7-carboxamide;
N-(5-((2-(4-azaspiro[2.4]heptan-4-yl)ethyl)carbamoyl)-3-methylthiophen-2-yl)-2-(1-methyl-1H-pyrazol-4-yl)pyrazolo[5,1-b]thiazole-7-carboxamide; or
N-(5-(3-(2-(2,2-dimethylpyrrolidin-1-yl)ethyl)ureido)-2-methyl-pyridin-3-yl)-2-(1-methyl-1H-pyrazol-4-yl)pyrazolo[5,1-b]thiazole-7-carboxamide.
   Aspect 72. The compound according to aspect 1 or aspect 2, wherein said compound is:
2-(6,7-dihydro-5H-pyrazolo[5,1-b][1,3]oxazin-3-yl)-N-(5-(2-(3,3-dimethylazetidin-1-yl)acetamido)-2-methylpyridin-3-yl)pyrazolo[5,1-b]thiazole-7-carboxamide;
N-(5-(2-(3,3-dimethylazetidin-1-yl)acetamido)-2-methylpyridin-3-yl)-2-(4-methoxypyridin-3-yl)pyrazolo[5,1-b]thiazole-7-carboxamide;
N-(5-(3-(2,2-dimethylpyrrolidin-1-yl)propanamido)-2-methyl pyridin-3-yl)-2-(1-(2-methoxyethyl)-1H-pyrazol-4-yl)pyrazolo[5,1-b]thiazole-7-carboxamide;
N-(5-(3-(2,2-dimethylpyrrolidin-1-yl)propanamido)-2-methyl-pyridin-3-yl)-2-(1-methyl-1H-pyrazol-4-yl)pyrazolo[5,1-b]thiazole-7-carboxamide; or
N-(5-(3-(2-azabicyclo[2.2.2]octan-2-yl)propanamido)-2-methyl pyridin-3-yl)-2-(1-methyl-1H-pyrazol-4-yl)pyrazolo[5,1-b]thiazole-7-carboxamide.
   Aspect 73. A pharmaceutical composition comprising a compound according to any one of aspects 1-72, or a pharmaceutically acceptable salt thereof, and a pharmaceutically acceptable excipient.
   Aspect 74. A method of treating a disease or disorder in which PDGFR signaling is implicated in a subject in need thereof, comprising administering to said subject an amount of a compound according to any one of aspects 1-72, or a pharmaceutically acceptable salt thereof, that is effective to treat said disease or disorder.
   Aspect 75. The method according to aspect 74, wherein said disease or disorder is pulmonary hypertension (PH).
   Aspect 76. The method according to aspect 75, wherein said pulmonary hypertension is pulmonary arterial hypertension (PAH); PH secondary to heart failure; PH secondary to lung diseases and/or hypoxia; PH due to pulmonary artery obstruction; or PH due to unknown or rare diseases.
   Aspect 77. The method according to aspect 75, wherein said pulmonary hypertension is pulmonary arterial hypertension (PAH).

1. A compound of formula (I₀): 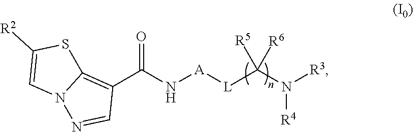
or a pharmaceutically acceptable salt thereof, wherein
A is an optionally substituted phenyl ring, an optionally substituted pyridinyl ring, or an optionally substituted 5-membered heteroaryl ring containing 1-2 heteroatoms that are each independently O, N, or S;
R² is optionally substituted aryl, optionally substituted heteroaryl, optionally substituted fused heterocycloalkyl, optionally substituted alkyl, optionally substituted alkenyl, optionally substituted cycloalkyl, or optionally substituted heterocycloalkyl;
R³ and R⁴ are each independently optionally substituted aryl, optionally substituted heteroaryl, optionally substituted alkyl, optionally substituted cycloalkyl, or optionally substituted heterocycloalkyl; or one of R³ or R⁴ may be H; or
R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R³ and R⁴ are attached, 1-3 other heteroatoms that are each independently O, S, or N;
each R⁵ and each R⁶ is independently H, C₁-C₆alkyl, or C₃-C₅cycloalkyl; or
an R⁵ and R⁶ attached to the same carbon atom, together with that carbon atom, may form a C₃-C₆cycloalkyl ring; or
an R⁵ and R⁶ attached to the same carbon atom, together with that carbon atom, may form a C═O; or
an R⁵ or R⁶, together with an R³ or R⁴ may form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system;
n is 1, 2, 3, 4, or 5; and
L is —NHC(O)— or 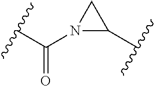 [SMILES: *C(=O)N1CC1*]
when n is 1; or —NHC(O)—, —NHS(O)₂—, 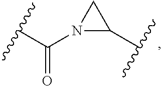 [SMILES: *C(=O)N1CC1*]
—NHC(O)O—, —S(O)₂NH—, —C(O)NH—, or —NHC(O)NH when n is 2, 3, 4, or 5.

2. The compound according to claim 1, wherein the compound of formula (Io) is a compound of formula (I): 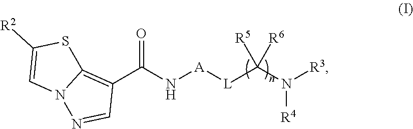
or a pharmaceutically acceptable salt thereof, wherein A in the compound of formula (I) is an optionally substituted phenyl ring, an optionally substituted pyridinyl ring, or an optionally substituted 5-membered heteroaryl ring containing 1-2 heteroatoms that are each independently O, N, or S; R² is optionally substituted aryl, optionally substituted heteroaryl, optionally substituted alkyl, optionally substituted cycloalkyl, or optionally substituted heterocycloalkyl; R³ and R⁴ are each independently optionally substituted aryl, optionally substituted heteroaryl, optionally substituted alkyl, optionally substituted cycloalkyl, or optionally substituted heterocycloalkyl; or R³ and R⁴, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system optionally includes, in addition to the nitrogen atom to which both R³ and R⁴ are attached, 1-3 other heteroatoms that are each independently O, S, or N; each R⁵ and each R⁶ is independently H, C₁-C₆alkyl, C₃-C₅cycloalkyl, or an R⁵ and R⁶ attached to the same carbon atom, together with that carbon atom, may form a C₃-C₆cycloalkyl ring; n is 1, 2, or 3; and when n is 1, L is —NHC(O)—, and when n is 2 or 3, L is —C(O)NH—, —NHC(O)—, or —NHC(O)NH.

3. The compound of formula (I) according to claim 2, wherein said compound is a compound of formula (IA) or formula (IB): 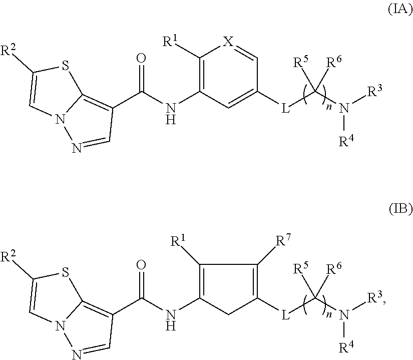
or a pharmaceutically acceptable salt thereof,
wherein
R¹ is H, C₁-C₆alkyl, C₃-C₆cycloalkyl, halogen, —CN, or C₁-C₄fluoroalkyl;
R⁷ is H, C₁-C₆alkyl, C₃-C₆cycloalkyl, halogen, —CN, or —CF₃; and
X is N, or CH.

4. (canceled)

5. (canceled)

6. (canceled)

7. (canceled)

8. The compound according to claim 1, wherein R¹ is C₁-C₆alkyl.

9. The compound according to claim 1, wherein R² is optionally substituted heteroaryl.

10. (canceled)

11. (canceled)

12. (canceled)

13. (canceled)

14. The compound according to claim 1, wherein L is —C(O)NH—.

15. The compound according to claim 1, wherein L is —NHC(O)—.

16. The compound according to claim 1, wherein L is —NHC(O)NH—.

17. The compound according to claim 15, wherein n is 1.

18. The compound according to claim 1, wherein n is 2.

19. The compound according to claim 1, wherein n is 3.

20. The compound according to claim 1, wherein each R⁵ and each R⁶ is H.

21. The compound according to claim 1, wherein R³ is optionally substituted alkyl and R⁴ is optionally substituted heterocycloalkyl.

22. The compound according to claim 1, wherein R³ and R⁴, together with the nitrogen atom to which they are both attached, form an optionally substituted 3-12-membered heterocycloalkyl ring, an optionally substituted 5-12-membered bridged heterocycloalkyl ring; an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, and 5-12-membered spiroheterocycloalkyl ring system optionally includes, in addition to the nitrogen atom to which both R³ and R⁴ are attached, 1-3 heteroatoms that are each independently O, S, or N.

23. (canceled)

24. (canceled)

25. (canceled)

26. (canceled)

27. The compound according to claim 2, wherein said compound is:
N-(5-((2-(2-azabicyclo[2.2.2]octan-2-yl)ethyl)carb-amoyl)-2-methylpyridin-3-yl)-2-(1-methyl-1H-pyrazol-4-yl)pyrazolo[5,1-b]thiazole-7-carboxamide;
N-(5-((2-(4-azaspiro[2.4]heptan-4-yl)ethyl)carbamoyl)-2-methylpyridin-3-yl)-2-(1-methyl-1H-pyrazol-4-yl)pyrazolo[5,1-b]thiazole-7-carboxamide;
N-(5-((2-(5-azaspiro[3.4]octan-5-yl)ethyl)carbamoyl)-2-methylpyridin-3-yl)-2-(1-methyl-1H-pyrazol-4-yl)pyrazolo[5,1-b]thiazole-7-carboxamide;
N-(5-((2-(9-azabicyclo[3.3.1]nonan-9-yl)ethyl)carbamoyl)-2-methylpyridin-3-yl)-2-(1-methyl-1H-pyrazol-4-yl)pyrazolo[5,1-b]thiazole-7-carboxamide;
N-(5-((2-(3-azabicyclo[3.1.1]heptan-3-yl)ethyl)carbamoyl)-2-methylpyridin-3-yl)-2-(1-methyl-TH-pyrazol-4-yl)pyrazolo[5,1-b]thiazole-7-carboxamide;
2-(1-cyclopropyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide;
N-(5-((2-(2,2-dimethylpyrrolidin-1-yl)ethyl)carbamoyl)-2-methyl-pyridin-3-yl)-2-(pyridin-4-yl)pyrazolo[5,1-b]thiazole-7-carboxamide;
N-(5-((2-(2,2-dimethylpyrrolidin-1-yl)ethyl)carbamoyl)-2-methylpyridin-3-yl)-2-(1-methyl-TH-pyrazol-4-yl)pyrazolo[5,1-b]thiazole-7-carboxamide;
N-(5-((2-(2,2-dimethylpyrrolidin-1-yl)ethyl)carbamoyl)-2-methylpyridin-3-yl)-2-(1-methyl-TH-pyrazol-3-yl)pyrazolo[5,1-b]thiazole-7-carboxamide;
N-(5-(3-(2,2-dimethylpyrrolidin-1-yl)propanamido)-2-methyl-pyridin-3-yl)-2-(1-methyl-TH-pyrazol-4-yl)pyrazolo[5,1-b]thiazole-7-carboxamide;
N-(5-(3-(2,2-dimethylpyrrolidin-1-yl)propanamido)-2-methyl-pyridin-3-yl)-2-(1-methyl-TH-pyrazol-5-yl)pyrazolo[5,1-b]thiazole-7-carboxamide;
N-(5-(3-(2,2-dimethylpyrrolidin-1-yl)propanamido)-2-methyl-pyridin-3-yl)-2-(1-methyl-TH-pyrazol-3-yl)pyrazolo[5,1-b]thiazole-7-carboxamide;
N-(5-(2-(2,2-dimethylpyrrolidin-1-yl)acetamido)-2-methylpyridin-3-yl)-2-(1-methyl-1H-pyrazol-4-yl)pyrazolo[5,1-b]thiazole-7-carboxamide;
N-(5-(2-(4,4-difluoropiperidin-1-yl)acetamido)-2-methylpyridin-3-yl)-2-(1-methyl-1H-pyrazol-4-yl)pyrazolo[5,1-b]thiazole-7-carboxamide;
2-(1-methyl-1H-pyrazol-4-yl)-N-(2-methyl-5-(2-(methyl(tetrahydro-2H-pyran-4-yl)amino)acetamido)pyridin-3-yl)pyrazolo[5,1-b]thiazole-7-carboxamide;
N-(5-(2-(4-azaspiro[2.4]heptan-4-yl)acetamido)-2-methylpyridin-3-yl)-2-(1-methyl-1H-pyrazol-4-yl)pyrazolo[5,1-b]thiazole-7-carboxamide;
2-(1-methyl-1H-pyrazol-4-yl)-N-(2-methyl-5-(2-(1-methyl-4,5-dihydro-1H-pyrazolo[3,4-c]pyridin-6 (7H)-yl)acetamido)pyridin-3-yl)pyrazolo[5,1-b]thiazole-7-carboxamide;
N-(5-(2-(2-oxa-6-azaspiro[3.4]octan-6-yl)acetamido)-2-methylpyridin-3-yl)-2-(1-methyl-1H-pyrazol-4-yl)pyrazolo[5,1-b]thiazole-7-carboxamide;
N-(5-(2-(2-oxa-7-azaspiro[4.4]nonan-7-yl)acetamido)-2-methylpyridin-3-yl)-2-(1-methyl-1H-pyrazol-4-yl)pyrazolo[5,1-b]thiazole-7-carboxamide;
N-(5-(2-(2-oxa-5-azaspiro[3.4]octan-5-yl)acetamido)-2-methyl-pyridin-3-yl)-2-(1-methyl-1H-pyrazol-4-yl)pyrazolo[5,1-b]thiazole-7-carboxamide;
N-(5-(2-(1-oxa-7-azaspiro[4.4]nonan-7-yl)acetamido)-2-methylpyridin-3-yl)-2-(1-methyl-1H-pyrazol-4-yl)pyrazolo[5,1-b]thiazole-7-carboxamide;
N-(5-(2-(3,3-dimethylazetidin-1-yl)acetamido)-2-methylpyridin-3-yl)-2-(1-methyl-1H-pyrazol-4-yl)pyrazolo[5,1-b]thiazole-7-carboxamide;
N-(5-(2-(3,4-dihydro-2,7-naphthyridin-2 (1H)-yl)acetamido)-2-methylpyridin-3-yl)-2-(1-methyl-1H-pyrazol-4-yl)pyrazolo[5,1-b]thiazole-7-carboxamide;
N-(5-(2-(2-oxa-6-azaspiro[3.5]nonan-6-yl)acetamido)-2-methylpyridin-3-yl)-2-(1-methyl-1H-pyrazol-4-yl)pyrazolo[5,1-b]thiazole-7-carboxamide;
N-(5-(2-(2-oxa-5-azaspiro[3.5]nonan-5-yl)acetamido)-2-methylpyridin-3-yl)-2-(1-methyl-1H-pyrazol-4-yl)pyrazolo[5,1-b]thiazole-7-carboxamide;
(R)-2-(1-methyl-1H-pyrazol-4-yl)-N-(2-methyl-5-(2-(2-methyl-pyrrolidin-1-yl)acetamido)pyridin-3-yl)pyrazolo[5,1-b]thiazole-7-carboxamide;
(S)-2-(1-methyl-1H-pyrazol-4-yl)-N-(2-methyl-5-(2-(2-methyl-pyrrolidin-1-yl)acetamido)pyridin-3-yl)pyrazolo[5,1-b]thiazole-7-carboxamide;
N-(5-(2-(3,3-dimethylpyrrolidin-1-yl)acetamido)-2-methylpyridin-3-yl)-2-(1-(2-hydroxyethyl)-1H-pyrazol-4-yl)pyrazolo[5,1-b]thiazole-7-carboxamide;
N-(5-(2-(5-azaspiro[3.4]octan-5-yl)acetamido)-2-methylpyridin-3-yl)-2-(1-(2-hydroxyethyl)-1H-pyrazol-4-yl)pyrazolo[5,1-b]thiazole-7-carboxamide;
N-(5-(2-(6-azaspiro[3.4]octan-6-yl)acetamido)-2-methylpyridin-3-yl)-2-(1-(2-hydroxyethyl)-1H-pyrazol-4-yl)pyrazolo[5,1-b]thiazole-7-carboxamide;
N-(5-(2-(6-azaspiro[2.5]octan-6-yl)acetamido)-2-methylpyridin-3-yl)-2-(1-(2-hydroxyethyl)-1H-pyrazol-4-yl)pyrazolo[5,1-b]thiazole-7-carboxamide;
N-(5-(2-(5-azaspiro[2.4]heptan-5-yl)acetamido)-2-methylpyridin-3-yl)-2-(1-(2-hydroxyethyl)-1H-pyrazol-4-yl)pyrazolo[5,1-b]thiazole-7-carboxamide;
(R)-2-(1-(2-hydroxyethyl)-1H-pyrazol-4-yl)-N-(2-methyl-5-(2-(2-methyl-piperidin-1-yl)acetamido)pyridin-3-yl)pyrazolo[5,1-b]thiazole-7-carboxamide;
(S)-2-(1-(2-hydroxyethyl)-1H-pyrazol-4-yl)-N-(2-methyl-5-(2-(2-methyl-piperidin-1-yl)acetamido)pyridin-3-yl)pyrazolo[5,1-b]thiazole-7-carboxamide;
N-(5-((2-(5-azaspiro[3.4]octan-5-yl)ethyl)carbamoyl)-3-methylthiophen-2-yl)-2-(1-methyl-TH-pyrazol-4-yl)pyrazolo[5,1-b]thiazole-7-carboxamide;
N-(5-((2-(2,2-dimethylpyrrolidin-1-yl)ethyl)carbamoyl)-3-methylthiophen-2-yl)-2-(pyridin-3-yl)pyrazolo[5,1-b]thiazole-7-carboxamide;
N-(5-((2-(4-azaspiro[2.4]heptan-4-yl)ethyl)carbamoyl)-3-methylthiophen-2-yl)-2-(1-methyl-TH-pyrazol-4-yl)pyrazolo[5,1-b]thiazole-7-carboxamide; or
N-(5-(3-(2-(2,2-dimethylpyrrolidin-1-yl)ethyl)ureido)-2-methyl-pyridin-3-yl)-2-(1-methyl-TH-pyrazol-4-yl)pyrazolo[5,1-b]thiazole-7-carboxamide.

28. The compound according to claim 2, wherein said compound is:
2-(6,7-dihydro-5H-pyrazolo[5,1-b][1,3]oxazin-3-yl)-N-(5-(2-(3,3-dimethylazetidin-1-yl)acetamido)-2-methylpyridin-3-yl)pyrazolo[5,1-b]thiazole-7-carboxamide;
N-(5-(2-(3,3-dimethylazetidin-1-yl)acetamido)-2-methylpyridin-3-yl)-2-(4-methoxypyridin-3-yl)pyrazolo[5,1-b]thiazole-7-carboxamide;
N-(5-(3-(2,2-dimethylpyrrolidin-1-yl)propanamido)-2-methyl pyridin-3-yl)-2-(1-(2-methoxyethyl)-1H-pyrazol-4-yl)pyrazolo[5,1-b]thiazole-7-carboxamide;
N-(5-(3-(2,2-dimethylpyrrolidin-1-yl)propanamido)-2-methyl-pyridin-3-yl)-2-(1-methyl-TH-pyrazol-4-yl)pyrazolo[5,1-b]thiazole-7-carboxamide; or
N-(5-(3-(2-azabicyclo[2.2.2]octan-2-yl)propanamido)-2-methyl pyridin-3-yl)-2-(1-methyl-TH-pyrazol-4-yl)pyrazolo[5,1-b]thiazole-7-carboxamide.

29. The compound according to claim 1, wherein said compound is:
2-(6,7-dihydro-5H-pyrazolo[5,1-b][1,3]oxazin-3-yl)-N-(5-(2-(3,3-dimethylazetidin-1-yl)acetamido)-2-methylpyridin-3-yl)pyrazolo[5,1-b]thiazole-7-carboxamide;
N-(5-(3-(2,2-dimethylpyrrolidin-1-yl)propanamido)-2-methyl pyridin-3-yl)-2-(1-(2-methoxyethyl)-1H-pyrazol-4-yl)pyrazolo[5,1-b]thiazole-7-carboxamide;
N-(5-(3-(2-azabicyclo[2.2.2]octan-2-yl)propanamido)-2-methyl pyridin-3-yl)-2-(1-methyl-TH-pyrazol-4-yl)pyrazolo[5,1-b]thiazole-7-carboxamide;
N-(5-((2-(1-azaspiro[3.3]heptan-1-yl)ethyl)carbamoyl)-2-methylpyridin-3-yl)-2-(1,5-dimethyl-1H-pyrazol-4-yl)pyrazolo[5,1-b]thiazole-7-carboxamide;
2-(1,5-dimethyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide;
2-(1,3-dimethyl-1H-pyrazol-4-yl)-N-(5-((2-(2,2-dimethylpyrrolidin-1-yl)ethyl)carbamoyl)-2-methylpyridin-3-yl)pyrazolo[5,1-b]thiazole-7-carboxamide;
N-(5-((2-(2-azabicyclo[2.2.2]octan-2-yl)ethyl)carbamoyl)-2-methylpyridin-3-yl)-2-(1,3-dimethyl-1H-pyrazol-4-yl)pyrazolo[5,1-b]thiazole-7-carboxamide;
N-(5-((2-(5-azaspiro[3.4]octan-5-yl)ethyl)carbamoyl)-2-methyl-pyridin-3-yl)-2-(1,5-dimethyl-1H-pyrazol-4-yl)pyrazolo[5,1-b]thiazole-7-carboxamide;
N-(5-((2-(5-azaspiro[3.4]octan-5-yl)ethyl)carbamoyl)-2-methylpyridin-3-yl)-2-(1,3-dimethyl-1H-pyrazol-4-yl)pyrazolo[5,1-b]thiazole-7-carboxamide;
N-(5-(2-(3,3-dimethylazetidin-1-yl)acetamido)-2-methylpyridin-3-yl)-2-(1-(oxetan-3-yl)-1H-pyrazol-4-yl)pyrazolo[5,1-b]thiazole-7-carboxamide;
N-(5-(2-(2,2-dimethylpyrrolidin-1-yl)acetamido)-2-methylpyridin-3-yl)-2-(1-(1-hydroxy-2-methylpropan-2-yl)-1H-pyrazol-4-yl)pyrazolo[5,1-b]thiazole-7-carboxamide;
2-(6,7-dihydro-4H-pyrazolo[5,1-c][1,4]oxazin-3-yl)-N-(5-(2-(2,2-dimethylpyrrolidin-1-yl)acetamido)-2-methylpyridin-3-yl)pyrazolo[5,1-b]thiazole-7-carboxamide;
N-(5-((2-(5-azaspiro[3.4]octan-5-yl)ethyl)carbamoyl)-2-methylpyridin-3-yl)-2-(5,6-dihydro-4H-pyrrolo[1,2-b]pyrazol-3-yl)pyrazolo[5,1-b]thiazole-7-carboxamide;
N-(5-(2-(cyclobutylamino)acetamido)-2-methylpyridin-3-yl)-2-(5,6-dihydro-4H-pyrrolo[1,2-b]pyrazol-3-yl)pyrazolo[5,1-b]thiazole-7-carboxamide;
N-(5-((2-(5-azaspiro[3.4]octan-5-yl)ethyl)carbamoyl)-2-methylpyridin-3-yl)-2-(6,7-dihydro-4H-pyrazolo[5,1-c][1,4]oxazin-3-yl)pyrazolo[5,1-b]thiazole-7-carboxamide;
(S)-2-(6,7-dihydro-4H-pyrazolo[5,1-c][1,4]oxazin-3-yl)-N-(2-methyl-5-(2-(2-methylpyrrolidin-1-yl)acetamido)pyridin-3-yl)pyrazolo[5,1-b]thiazole-7-carboxamide;
(S)—N-(5-(2-(1-isopropylpyrrolidin-3-yl)acetamido)-2-methylpyridin-3-yl)-2-(1-(2-methoxyethyl)-1H-pyrazol-4-yl)pyrazolo[5,1-b]thiazole-7-carboxamide; or
2-(5,6-dihydro-4H-pyrrolo[1,2-b]pyrazol-3-yl)-N-(5-((2-(3,3-dimethylazetidin-1-yl)ethyl)carbamoyl)-2-methylpyridin-3-yl)pyrazolo[5,1-b]thiazole-7-carboxamide; or
a pharmaceutically acceptable salt of one of these compounds.

30. (canceled)

31. A compound according to claim 1, wherein the compound has an IC₅₀<20 nM in a PDGFR cellular assay.

32. The compound according to claim 31, wherein the compound has an IC₅₀<5 nM in a PDGFR cellular assay.

33. A pharmaceutical composition comprising a compound according to claim 1, or a pharmaceutically acceptable salt thereof, and a pharmaceutically acceptable excipient.

34. A method of treating a disease or disorder in which PDGFR signaling is implicated in a subject in need thereof, comprising administering to said subject an amount of a compound according to claim 1, or a pharmaceutically acceptable salt thereof, that is effective to treat said disease or disorder.

35. The method according to claim 34, wherein said disease or disorder is pulmonary hypertension (PH).

36. The method according to claim 35, wherein said pulmonary hypertension is pulmonary arterial hypertension (PAH); PH secondary to heart failure; PH secondary to lung diseases and/or hypoxia; PH due to pulmonary artery obstruction; or PH due to unknown or rare diseases.

37. The method according to claim 36, wherein said pulmonary hypertension is pulmonary arterial hypertension (PAH).
